(19) United States
(12) Patent Application Publication
(10) Pub. No.: US 2024/0360179 A1
(43) Pub. Date: Oct. 31, 2024

(54) STAPLED PEPTIDES AND METHODS THEREOF

(71) Applicant: FOG PHARMACEUTICALS, INC., Cambridge, MA (US)

(72) Inventors: Brian Halbert White, Malden, MA (US); Daniel Seungduk La, Chestnut Hill, MA (US); Lorenzo Josue Alfaro-Lopez, Belmont, MA (US); Paula Cristina Ortet, Malden, MA (US); Sarah Isabelle Cappucci, Tyngsboro, MA (US); Zhi Li, Braintree, MA (US); John Hanney McGee, Somerville, MA (US); Martin Robert Tremblay, Melrose, MA (US); Gregory L. Verdine, Boston, MA (US); Yaguang Si, Arlington, MA (US); Kevin Ling, Somerville, MA (US); Peicheng Du, Lexington, MA (US); Jonathan Barry Hurov, Bedford, MA (US)

(21) Appl. No.: 18/568,212

(22) PCT Filed: Jun. 8, 2022
(86) PCT No.: PCT/US2022/032738

§ 371 Date: Dec. 7, 2023

Related U.S. Application Data
(60) Provisional application No. 63/303,952, filed on Jan. 27, 2022.
(60) Provisional application No. 63/224,834, filed on Jul. 22, 2021.
(60) Provisional application No. 63/208,487, filed on Jun. 8, 2021.

(51) Int. Cl. C07K 7/56; A61K 38/00; A61K 45/06; A61P 35/00; C07K 1/107

(52) U.S. Cl. CPC C07K 7/56; A61K 45/06; A61P 35/00; C07K 1/1077; A61K 38/00

(57) ABSTRACT

Among other things, the present disclosure provides various useful agents. In some embodiments, provided agents can bind to beta-catenin. In some embodiments, the present disclosure provides technologies for modulating beta-catenin functions. In some embodiments, the present disclosure provides technologies for preventing and/or treating conditions, disorders or diseases associated with beta-catenin. In some embodiments, the present disclosure provides designed amino acids and agents which can provide improved properties and/or activities.

### CROSS-REFERENCE TO RELATED APPLICATIONS

This application claims priority to U.S. Provisional Application Nos. 63/208,487, filed Jun. 8, 2021, 63/224,834, filed Jul. 22, 2021, and 63/303,952, filed Jan. 27, 2022, the entirety of each of which is incorporated herein by reference.

### BACKGROUND

Stapled peptides are useful for various applications. For example, as biologically active agents, they can be utilized to modulate various biological functions.

### SUMMARY

Among other things, the present disclosure provides powerful technologies (e.g., agents (e.g., those that are or comprise peptides, in many embodiments, stapled peptides), compositions, methods, etc.) for modulating various biological functions.

In some embodiments, the present disclosure provides agents, e.g., stapled peptides that comprise multiple staples. In some embodiments, the present disclosure provides agents, e.g., stapled peptides that comprise three or more staples. In some embodiments, the present disclosure provides agents, e.g., stapled peptides that comprise three or more staples within 10-20 amino acid residues, e.g., 10-15, 11-15, 11-14, 11, 12, 13, 14, 15, 16, 17, 18, 19, or 20 consecutive amino acid residues. In some embodiments, the present disclosure provides agents, e.g., stapled peptides that comprise three or more staples within 11 consecutive amino acid residues. In some embodiments, the present disclosure provides agents, e.g., stapled peptides that comprise three or more staples within 14 consecutive amino acid residues. In some embodiments, within such numbers of amino acid residues there are three staples. In some embodiments, within such numbers of consecutive amino acid residues there are four staples. Without the intention to be limited by theory, in some embodiments, provided agents, e.g., stapled peptides have increased rigidity than reference peptides (e.g., unstapled peptides, or stapled peptides having fewer staples (in some embodiments, fewer staples within certain numbers of amino acid residues as described herein), etc.). In some embodiments, provided agents, e.g., stapled peptides demonstrate various desired properties and/or activities. In some embodiments, provided agents, e.g., stapled peptides provide improved desired properties and/or activities than reference peptides (e.g., unstapled peptides, or stapled peptides having fewer staples (in some embodiments, fewer staples within certain numbers of amino acid residues as described herein), etc.).

In some embodiments, provided technologies comprise designed structural features, e.g., novel amino acid residues, that can provide significantly improved properties and/or activities compared to comparable reference technologies that do not contain such designed structural features. In some embodiments, the present disclosure provides designed amino acids as described herein, whose incorporation into peptide agents, including stapled peptides, can provide significantly improved properties and/or activities such as improved lipophilicity and/or delivery into cells compared to reference amino acids (e.g., Asp). In some embodiments, the present disclosure provides technologies including peptides comprising such designed amino acid residues. In some embodiments, the present disclosure provides stapled peptides comprise such designed amino acid residues.

In some embodiments, the present disclosure provides technologies for modulating one or more functions of beta-catenin. Particularly, in some embodiments, the present disclosure provides various agents, e.g., peptides, in many instances stapled peptides, that can bind to beta-catenin and modulate its functions. As demonstrated herein, in some embodiments, the present disclosure binds agents that can interact with beta-catenin at a unique set of residues. In some embodiments, a binding site comprises one or more or all of the set of residues. In some embodiments, provided agents interact with one or more of a set of residues that are or correspond to the following residues of SEQ ID NO: 1: A305, Y306, G307, N308, Q309, K312, R342, K345, V346, V349, Q375, R376, Q379, N380, L382, W383, R386, N387, D413, N415, V416, T418, and C419. In some embodiments, provided agents interact with one or more of amino acid residue that are or correspond to A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, provided agents interact with one or more of amino acid residues that are or correspond to A305, Y306, G307, N308, Q309, K312, K345, V346, V349, Q379, N380, L382, W383, R386, N387, D413, N415, V416, T418, and C419 of SEQ ID NO: 1. In some embodiments, provided agents interact with one or more of amino acid residues that are or correspond to G307, K312, K345, W383, N387, D413, and N415 of SEQ ID NO: 1. In some embodiments, provided agents interact with one or more of amino acid residues that are or correspond to K312, K345, R386 and W383 of SEQ ID NO: 1. In some embodiments, provided agents interact with one or more of a set of residues that are or correspond to the following residues of SEQ ID NO: 1: G307, K312, K345, Q379, L382, W383, N387, N415, and V416. In some embodiments, provided agents interact with all of a set of residues that are or correspond to the following residues of SEQ ID NO: 1: Y306, G307, K312, K345, Q379, L382, W383, N387, N415, and V416. In some embodiments, provided agents interact with all of a set of residues that are or correspond to the following residues of SEQ ID NO: 1: G307, K312, K345, Q379, L382, W383, N387, N415, and V416. In some embodiments, provided agents interact with all of a set of residues that are or correspond to the following residues of SEQ ID NO: 1: Y306, G307, K312, K345, Q379, L382, W383, N387, N415, and V416. In some embodiments, provided agents interact with one or more of amino acid residues that are or correspond to K312, K345 and W383 of SEQ ID NO: 1. In some embodiments, provided agents interact with the amino acid residues that are or correspond to K312, K345 and W383 of SEQ ID NO: 1.

As demonstrated herein, provided technologies can modulate one or more biological processes associated with beta-catenin. In some embodiments, provided agents, e.g., stapled peptides, compete with a ligand (e.g., with a member of the T cell factor/lymphoid enhancer factor (TCF/LEF) family of transcription factors) for binding to beta-catenin. In some embodiments, provided agents compete with a ligand for binding to beta-catenin at a particular binding site (e.g., with a member of the T cell factor/lymphoid enhancer factor (TCF/LEF) family of transcription factors at the TCF site on beta-catenin). In some embodiments, provided technologies compete with TCF for interactions with beta-catenin. In some embodiments, binding of provided agents to a beta-catenin site decreases, suppresses and/or blocks binding to beta-catenin by another binding partner (e.g., a kinase). In some embodiments, binding of provided agents blocks binding of beta-catenin by a TCF/LEF family member. In some embodiments, the present disclosure provides agents that can bind to a site of beta-catenin selectively over one of more other binding sites by other ligands (e.g., peptides, proteins, etc.; in some embodiments, a ligand is Axin; in some embodiments, a ligand is Bcl9). In some embodiments, provided technologies modulate one or more beta-catenin functions associated with its interactions with TCF. In some embodiments, provided technologies selectively modulate beta-catenin functions, e.g., functions associated with TCF interactions. In some embodiments, provided technologies selectively modulate beta-catenin functions and do not significantly impact functions that are not associated with beta-catenin (e.g., various functions and/or processes in the Wnt pathway that are not associated with beta-catenin). In some embodiments, provided technologies are useful for inhibiting beta-catenin functions. In some embodiments, provided technologies are usefully for promoting and/or enhancing immune activities, e.g., anti-tumor adaptive immunity.

In some embodiments, provided technologies are useful for preventing or treating various conditions, disorders or diseases including cancer. In some embodiments, the present disclosure provides methods for treating or preventing a condition, disorder or disease associated with beta-catenin, comprising administering to a subject suffered therefrom or susceptible thereto an effective amount of a provided agent or a pharmaceutically acceptable salt thereof. In some embodiments, a condition, disorder or disease is associated with beta-catenin's interactions with TCF. In some embodiments, an agent, e.g., a staple peptide, is administered as a pharmaceutical composition. In some embodiments, the present disclosure provides pharmaceutical compositions which comprise or deliver a provided agent or a pharmaceutically acceptable salt thereof. In some embodiments, a pharmaceutical composition further comprises a lipid. As demonstrated herein, in some embodiments, a suitable lipid can promote delivery/activities. In some embodiments, an agent is or comprises a peptide. In some embodiments, an agent is or comprises a stapled peptides. In some embodiments, provided agents that can bind beta-catenin comprise one or more designed amino acid residues.

In some embodiments, the present disclosure provides agents that bind to a polypeptide comprising or consisting of SEQ ID NO: 1 (Uniprot ID P35222), or residues 250-450 of SEQ ID NO: 1, or residues 305-419 of SEQ ID NO: 1:

Uniprot No. P35222
(SEQ ID NO: 1)
MATQADLMELDMAMEPDRKAAVSHWQQQSYLDSGIHSGATTTAPSLSGKG
NPEEEDVDTSQVLYEWEQGFSQSFTQEQVADIDGQYAMTRAQRVRAAMFP
ETLDEGMQIPSTQFDAAHPTNVQRLAEPSQMLKHAVVNLINYQDDAELAT
RAIPELTKLLNDEDQVVVNKAAVMVHQLSKKEASRHAIMRSPQMVSAIVR
TMQNTNDVETARCTAGTLHNLSHHREGLLAIFKSGGIPALVKMLGSPVDS
VLFYAITTLHNLLLHQEGAKMAVRLAGGLQKMVALLNKTNVKFLAITTDC
LQILAYGNQESKLIILASGGPQALVNIMRTYTYEKLLWTTSRVLKVLSVC
SSNKPAIVEAGGMQALGLHLTDPSQRLVQNCLWTLRNLSDAATKQEGMEG
LLGTLVQLLGSDDINVVTCAAGILSNLTCNNYKNKMMVCQVGGIEALVRT
VLRAGDREDITEPAICALRHLTSRHQEAEMAQNAVRLHYGLPVVVKLLHP
PSHWPLIKATVGLIRNLALCPANHAPLREQGAIPRLVQLLVRAHQDTQRR
TSMGGTQQQFVEGVRMEEIVEGCTGALHILARDVHNRIVIRGLNTIPLFV
QLLYSPIENIQRVAAGVLCELAQDKEAAEAIEAEGATAPLTELLHSRNEG
VATYAAAVLFRMSEDKPQDYKKRLSVELTSSLFRTEPMAWNETADLGLDI
GAQGEPLGYRQDDPSYRSFHSGGYGQDALGMDPMMEHEMGGHHPGADYPV
DGLPDLGHAQDLMDGLPPGDSNQLAWFDTDL.

In some embodiments, provided agents specifically interact with one or more residues which are or correspond to residues 305-419 of SEQ ID NO: 1. In some embodiments, provided agents bind to a motif (e.g., a portion of a polypeptide, a domain of a polypeptide, etc.) that comprise one or more residues corresponding to Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419 of SEQ ID NO: 1. In some embodiments, provided agents bind to a motif (e.g., a portion of a polypeptide, a domain of a polypeptide, etc.) that comprise one or more residues corresponding to Ala305, Tyr306, Gly307, Asn 308, Gln309, Lys312, Lys345, Val346, Val349, Gln375, Arg376, Gln379, Asn380, Leu382, Trp383, Arg386, Asn387, Asp413, Asn415, Val416, Thr418, and Cys419 of SEQ ID NO: 1. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Lys345, Val346, Val349, Gln375, Arg376, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln 375, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Lys345, Val346, Val349, Gln379, Asn380, Leu382, Trp383, Arg386, Asn387, Asp413, Asn415, Val416, Thr418, and Cys419. In some embodiments, provided technologies bind to a motif comprising at least 2, 3, 4, 5, or 6 of G307, K312, K345, W383, N387, and N415. In some embodiments, provided technologies bind to a motif comprising at least 2, 3, 4, 5, 6, or 7 of G307, K312, K345, W383, N387, D413, and N415. In some embodiments, provided agents specifically bind to such motifs. In some embodiments, a motif may be referred to as a binding site. In some embodiments, provided technologies selectively bind to such a binding site over an Axin binding site. In some embodiments, provided technologies selectively bind to such a binding site over a Bcl9 binding site. In some embodiments, provided technologies selectively bind to such a binding site over a TCF binding site. In some embodiments, provided technology binds to such a binding site in a reverse N to C direction compared to TCF. In some embodiments, provided technologies do not bind to Axin binding site of beta-catenin. In some embodiments, provided technologies do not bind to Bcl9 binding site of beta-catenin. In some embodiments, provided technologies do not bind to ICAT binding site of beta-catenin. Various technologies, e.g., crystallography, NMR, biochemical assays, etc., may be utilized to assess interactions with beta-catenin in accordance with the present disclosure.

In some embodiments, the provided technology provides an agent, e.g., a stapled peptide, that comprises three staples within 10-20, 10-15, 10, 11, 12, 13, 14, 15, 16, 17, 18, 19, or 20 consecutive amino acids residues. In some embodiments, there are three or more staples within 10 consecutive amino acid residues. In some embodiments, there are three or more staples within 11 consecutive amino acid residues. In some embodiments, there are three or more staples within 12 consecutive amino acid residues. In some embodiments, there are three or more staples within 13 consecutive amino acid residues. In some embodiments, there are three or more staples within 14 consecutive amino acid residues. In some embodiments, there are three or more staples within 15 consecutive amino acid residues. In some embodiments, there are three or more staples within 16 consecutive amino acid residues. In some embodiments, there are three or more staples within 17 consecutive amino acid residues. In some embodiments, there are three or more staples within 18 consecutive amino acid residues. In some embodiments, there are three or more staples within 19 consecutive amino acid residues. In some embodiments, there are three or more staples within 20 consecutive amino acid residues. In some embodiments, two staples are bonded to the same amino acid residue. In some embodiments, two staples are bonded to the same backbone atom. In some embodiments, two staples are bonded to the same backbone carbon atom. In some embodiments, two staples are bonded to an alpha-carbon atom of an amino acid residue, and each independently bonds to another amino acid residue.

In some embodiments, a first staple in an agent, e.g., a staple peptide, are bonded to amino acid residues at positions i and i+3. In some embodiments, there is a second staple bonded to amino acid residues at positions i+3 and i+10. In some embodiments, there a third staple bonded to amino acid residues at positions i+9 and i+13. Those skilled in the art appreciate that as used in the art, i, i+3, i+9, i+10, i+13, etc. are routinely utilized to indicate relevant positions of amino acid residues. In some embodiments, they may also indicate absolute positions in an agent, e.g., a peptide. In some embodiments, i is an integer of 1-50 (e.g., 1, 2, 3, 4, 5, 6, 7, 8, 9, 10, 11, 12, 13, 14, 15, 16, 17, 18, 19, or 20). In some embodiments, i is 1. In some embodiments, there is a fourth staple in an agent, e.g., a stapled peptide.

In some embodiments, there are two amino acid residues between two amino acid residues bonded to the same staple. Such a staple may be referred to as a (i, i+3) staple. Similarly, in some embodiments, there are 3, 4, 5, 6, 7, 8, 9, or 10 amino acid residues between two amino acid residues bonded to the same staple, and such a staple may be referred to as a (i, i+4), (i, i+5), (i, i+6), (i, i+7), (i, i+8), (i, i+9), (i, i+10), or (i, i+11) staple, respectively.

In some embodiments, an agent, e.g., a stapled peptide, comprises a (i, i+2) staple and a (i, i+7) staple. In some embodiments, an agent, e.g., a stapled peptide, comprises a (i, i+3) staple and a (i, i+7) staple. In some embodiments, a (i, i+3) staple and (i, i+7) staple are bonded to the same amino acid residue. In some embodiments, a (i, i+3) staple and (i, i+7) staple bond to the same atom. In some embodiments, a (i, i+3) staple and (i, i+7) staple bond to the same alpha carbon atom. For example, in compound I-1, a (i, i+3) staple is bonded to amino acid residues at positions 1 and 4, and a (i, i+7) staple is bonded to amino acid residues at positions 4 and 11, and the two staples are both bonded to the alpha carbon of the amino acid residue at position 4. In some embodiments, an agent further comprises a third staple. In some embodiments, a third staple is (i, i+4). In some embodiments, a third staple is (i, i+7). In some embodiments, a third staple is not bonded to any of the amino acid residues that are bonded to the first two staples. In some embodiments, an agent further comprises a fourth staple. In some embodiments, a fourth staple is (i, i+4). In some embodiments, a fourth staple is (i, i+7). In some embodiments, a fourth staple is not bonded to any of the amino acid residues that are bonded to the first two staples. In some embodiments, a fourth staple is not bonded to any of the amino acid residues that are bonded to the first third staples.

In some embodiments, a provided agent, e.g., a peptide agent such as a stapled peptide agent, comprises one or more (e.g., 1, 2, 3, 4, 5, 6, or 7) of the following groups (in some embodiments, from the N to C direction):
a first acidic group (e.g., of a first acidic amino acid residue);
a second acidic group (e.g., of a second acidic amino acid residue);
optionally a third acidic group (e.g., of a third acidic amino acid residue);
optionally a hydrophobic group (e.g., of a hydrophobic amino acid residue)
a first aromatic group (e.g., of a first aromatic amino acid residue);
a second aromatic group (e.g., of a first aromatic amino acid residue); and
a third aromatic group (e.g., of a third aromatic amino acid residue).
   In some embodiments, an agent comprises a first and second acidic group and a first, second and third aromatic group. In some embodiments, such an agent additionally comprises a third acidic group (e.g., of a third acid amino acid residue) and/or a hydrophobic group (e.g., of a hydrophobic amino acid residue). In some embodiments, such an agent additionally comprises a third acidic group (e.g., of a third acid amino acid residue) and a hydrophobic group (e.g., of a hydrophobic amino acid residue). In some embodiments, the distance between a first acidic group and a second acidic group is about the distance between the acidic groups of two acidic amino acid residues of a peptide motif, wherein there are two amino acid residues between the two acidic amino acid residues (e.g., if the first acidic amino acid residue is at position N, the second is at position N+3), the distance between a first acidic group and a third acidic group (if present) is about the distance between the acidic groups of two acidic amino acid residues of a peptide motif, wherein there are three amino acid residues between the two acidic amino acid residues (e.g., if the first acidic amino acid residue is at position N, the third is at position N+4), the distance between a first acidic group and a hydrophobic group (if present) is about the distance between the acidic group of an acidic amino acid residue and the hydrophobic group of a hydrophobic amino acid residue of a peptide motif, wherein there are five amino acid residues between the first acidic amino acid residue and the hydrophobic amino acid residue (e.g., if the first acidic amino acid residue is at position N, the hydrophobic amino acid residue is at position N+6), the distance between a first acidic group and a first aromatic group is about the distance between the acidic group of a first acidic amino acid residue and the aromatic group of an aromatic amino acid residue of a peptide motif, wherein there are six amino acid residues between the first acidic amino acid residue and the first aromatic amino acid residue (e.g., if the first acidic amino acid residue is at position N, the first aromatic amino acid residue is at position N+7), the distance between the first aromatic group and the second aromatic group is about the distance between the aromatic groups of two aromatic amino acid residues of a peptide motif, wherein there are two amino acid residues between the two aromatic amino acid residues (e.g., if the first aromatic amino acid residue is at position M, the second is at position M+3), and/or the distance between the first aromatic group and the third aromatic group is about the distance between the aromatic groups of two aromatic amino acid residues of a peptide motif, wherein there are three amino acid residues between the two aromatic amino acid residues (e.g., if the first aromatic amino acid residue is at position M, the third is at position M+4). In some embodiments, a first acidic amino acid residue is at position N, a second acidic amino acid residue is at position N+3, and a first, second and third aromatic amino acid residue are at positions N+7, N+10 and N+11, respectively. In some embodiments, a first acidic amino acid residue is at position N, a second acidic amino acid residue is at position N+3, a third acidic amino acid residue is at position N+4, and a first, second and third aromatic amino acid residue are at positions N+7, N+10 and N+11, respectively. In some embodiments, a first acidic amino acid residue is at position N, a second acidic amino acid residue is at position N+3, a hydrophobic amino acid residue is at position N+6, and a first, second and third aromatic amino acid residue are at positions N+7, N+10 and N+11, respectively. In some embodiments, a first acidic amino acid residue is at position N, a second acidic amino acid residue is at position N+3, a third acidic amino acid residue is at position N+4, a hydrophobic amino acid residue is at position N+6, and a first, second and third aromatic amino acid residue are at positions N+7, N+10 and N+11, respectively. In some embodiments, M is N+7. In some embodiments, N is 1-7. In some embodiments, N is 1, 2, 3, 4, or 5. In some embodiments, N is 1. In some embodiments, N is 2. In some embodiments, N is 3. In some embodiments, N is 4. In some embodiments, N is 5. In some embodiments, M is 8-16. In some embodiments, M is 8. In some embodiments, M is 9. In some embodiments, M is 10. In some embodiments, M is 11. In some embodiments, M is 12. In some embodiments, M is 13. In some embodiments, a peptide motif is an alpha-helical motif wherein each amino acid residue is independently an alpha amino acid residue. In some embodiments, a peptide motif is stapled. In some embodiments, there are two or more staples in a peptide motif; in some embodiments, there are three; in some embodiments, there are four; in some embodiments, there are four or more. In some embodiments, a peptide motif is or comprises an agent described in a Table herein (e.g., I-xxxx wherein xxxx is a number (e.g., I-1, I-10, I-100, I-1000, etc.)). In some embodiments, a first acidic group is of X² as described herein, a second acidic group is of X⁵ as described herein, a third acidic group (if present) is of X⁶ as described herein, a hydrophobic group (if present) is of X⁸ as described herein, a first aromatic group is of X⁹ as described herein, a second aromatic group is of X² as described herein, and/or a third aromatic group is of X¹³ as described herein. In some embodiments, as described herein, a provided agent is a stapled peptide comprising one or more staples. In some embodiments, as described herein, a provided agent is a stapled peptide comprising two or more staples. In some embodiments, as described herein, a provided agent is a stapled peptide comprising three or more staples. In some embodiments, when contacted with a beta-catenin polypeptide, a first acidic group interacts with Lys312 and/or Gly307 or amino acid residues corresponding thereto, a second acidic group interacts with Asn387, Trp383 and/or Arg386 or amino acid residues corresponding thereto, a first aromatic group interacts with Lys345 and/or Trp383 or amino acid residues corresponding thereto, a second aromatic group interacts with Trp383 and/or Asn415 or amino acid residues corresponding thereto, and a third aromatic group interacts with Gln379, Leu383, Val416, Asn415 and/or Trp383 or amino acid residues corresponding thereto. In some embodiments, a third acidic group interacts with Asn387, Trp383 and/or Arg386 or amino acid residues corresponding thereto. In some embodiments, a hydrophobic group interacts with Trp383 or an amino acid residue corresponding thereto.

In some embodiments, the present disclosure provides an agent having the structure of formula I:

R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I

or a salt thereof, wherein each variable is independently as described herein.

In some embodiments, the present disclosure provides an agent which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent which is or comprises a peptide comprising:

[X]ₚX¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]_{p′},

wherein:
each of p15, p16 and p17 is independently 0 or 1;
each of p and p′ is independently 0-10;
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, an agent is R^{N}—[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′-R^{C}, wherein each variable is independently as described herein.

In some embodiments, an agent is or comprises X¹X²X³X⁴X⁵X⁶X⁷X⁸X⁹X¹⁰X¹¹X¹²X¹³[X¹⁴]ₚ₁₄][X¹⁵]ₚ₁₅[X¹⁶]ₚ₁₆[X¹⁷]ₚ₁₇[X¹⁸]ₚ₁₈[X¹⁹]ₚ₁₉[X²⁰]ₚ₂₀[X²¹]ₚ₂₁[X²²]ₚ₂₂[X²³]ₚ₂₃, wherein each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue as described herein.

In some embodiments, such a peptide comprises three or more staples. In some embodiments, such a peptide comprises five or more residues suitable for stapling.

In some embodiments, the present disclosure provides an agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

In some embodiments, the present disclosure provides an agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X⁶ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

In some embodiments, an agent is or comprises a peptide. In some embodiments, an agent is or comprises a stapled peptide. In some embodiments, an agent is a peptide. In some embodiments, an agent is a stapled peptide. In some embodiments, an agent, a peptide, or a stapled peptide has the structure of [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇. In some embodiments, X¹ and X⁴, and/or X⁴ and X¹¹ are independently amino acid residues suitable for stapling, or are stapled, or X³ and X¹⁰ independently amino acid residues suitable for stapling, or are stapled. In some embodiments, X¹ and X⁴ are independently amino acid residues suitable for stapling. In some embodiments, X¹ and X⁴ are stapled. In some embodiments, X⁴ and X¹¹ are independently amino acid residues suitable for stapling. In some embodiments, X⁴ and X¹¹ are stapled. In some embodiments, X¹ and X⁴, and X⁴ and X¹¹ are independently amino acid residues suitable for stapling. In some embodiments, a stapled peptide is a stitched peptide comprising two or more staples, some of which may bond to the same backbone atom. In some embodiments, X¹ and X⁴ are stapled, and X⁴ and X¹¹ are stapled. In some embodiments, a staple connecting X¹ and X⁴ and a staple connecting X⁴ and X¹¹ are bonded to a common backbone atom of X⁴. In some embodiments, a common backbone atom is the alpha-carbon of X⁴. In some embodiments, X³ and X¹⁰ are independently amino acid residues suitable for stapling. In some embodiments, X³ and X¹⁰ are stapled. In some embodiments, X¹ and X³ are independently amino acid residues suitable for stapling. In some embodiments, X¹ and X³ are stapled. In some embodiments, X¹⁰ and X¹⁴ are independently amino acid residues suitable for stapling. In some embodiments, X¹⁰ and X¹⁴ are stapled. In some embodiments, X⁷ and X¹⁰ are independently amino acid residues suitable for stapling. In some embodiments, X⁷ and X¹⁰ are stapled. In some embodiments, X⁷ and X¹⁴ are independently amino acid residues suitable for stapling. In some embodiments, X⁷ and X¹⁴ are stapled. In some embodiments, X³ and X⁷ are independently amino acid residues suitable for stapling. In some embodiments, X³ and X⁷ are stapled.

In some embodiments, the present disclosure provides agents that bind to a polypeptide comprising or consisting of residues 305-419 of SEQ ID NO: 1 as described herein. In some embodiments, an agent, e.g., a peptide, has a molecular mass of no more than about 5000 Daltons. In some embodiments, it is no more than about 2500, 3000, 3500, 4000, 4500 or 5000 Daltons. In some embodiments, it is no more than about 2500 Daltons. In some embodiments, it is no more than about 3000 Daltons. In some embodiments, it is no more than about 3500 Daltons. In some embodiments, it is no more than about 4000 Daltons. In some embodiments, it is no more than about 500 Daltons.

In some embodiments, the present disclosure provides various technologies, e.g., reagents methods, etc., for preparing, characterizing, assessing and using provided agents and compositions thereof. In some embodiments, the present disclosure provides, e.g., methods, reagents and/or systems for identifying, characterizing and/or assessing provided agents and use thereof (e.g., as therapeutic or diagnostic agents).

In some embodiments, the present disclosure provides pharmaceutical compositions comprising or delivering a provided agent and a pharmaceutical acceptable carrier. In some embodiments, a provided agent is a pharmaceutically acceptable salt form. In some embodiments, a provided composition comprises a pharmaceutically acceptable salt form an agent. In some embodiments, in various compositions and methods, agents are provided as pharmaceutically acceptable salt forms.

In some embodiments, the present disclosure provides methods for modulating a property, activity and/or function of beta-catenin, comprising contacting beta-catenin with a provided agent. In some embodiments, the present disclosure provides methods for modulating a property, activity and/or function of beta-catenin in a system comprising beta-catenin, comprising administering to a system an effective amount of a provided agent. In some embodiments, the present disclosure provides methods for modulating a property, activity and/or function of beta-catenin in a system expressing beta-catenin, comprising administering or delivering to a system an effective amount of a provided agent. In some embodiments, an activity of beta-catenin is inhibited or reduced. In some embodiments, a function of beta-catenin is inhibited or reduced. In some embodiments, a property, activity and/or function is associated with beta-catenin/TCF interaction.

In some embodiments, the present disclosure provides methods for modulating beta-catenin/TCF interaction. In some embodiments, the present disclosure provides methods for modulating beta-catenin/TCF interaction, comprising contacting beta-catenin with a provided agent. In some embodiments, the present disclosure provides methods for modulating beta-catenin/TCF interaction in a system comprising beta-catenin and TCF, comprising administering or delivering to the system an effective amount a provided agent. In some embodiments, the present disclosure provides methods for modulating beta-catenin/TCF interaction in a system expressing beta-catenin and TCF, comprising administering or delivering to the system an effective amount a provided agent. In some embodiments, interactions between beta-catenin and TCF is reduced. In some embodiments, interactions between beta-catenin and TCF is inhibited.

In some embodiments, the present disclosure provides methods for inhibiting cell proliferation, comprising administering or delivering to a population of cells an effective amount of a provided agent. In some embodiments, the present disclosure provides methods for inhibiting cell proliferation in a system, comprising administering or delivering to the system an effective amount of a provided agent. In some embodiments, the present disclosure provides methods for inhibiting cell growth, comprising administering or delivering to a population of cells an effective amount of a provided agent. In some embodiments, the present disclosure provides methods for inhibiting cell growth in a system, comprising administering or delivering to the system an effective amount of a provided agent. In some embodiments, such cell proliferation is beta-catenin dependent. In some embodiments, such cell growth is beta-catenin dependent. In some embodiments, such proliferation or growth is dependent on beta-catenin interactions with TCF.

In some embodiments, the present disclosure provides methods for reducing or preventing activation of a WNT pathway. In some embodiments, the present disclosure provides methods for reducing or preventing activation of a WNT pathway in a system, comprising administering or delivering to the system an effective amount of a provided agent.

In some embodiments, a system is in vitro. In some embodiments, a system is ex vivo. In some embodiments, a system is in vivo. In some embodiments, a system is or comprise a cell. In some embodiments, a system is or comprises a tissue. In some embodiments, a system is or comprises an organ. In some embodiments, a system is or comprises an organism. In some embodiments, a system is an animal. In some embodiments, a system is human. In some embodiments, a system is or comprises cells, tissues or organs associated with a condition, disorder or disease. In some embodiments, a system is or comprises cancer cells.

In some embodiments, the present disclosure provides methods for preventing conditions, disorders or diseases. In some embodiments, the present disclosure provides methods for reducing risks of conditions, disorders or diseases. In some embodiments, the present disclosure provides methods for preventing a condition, disorder or disease, comprising administering or delivering to a subject susceptible thereto an effective amount of an agent of the present disclosure. In some embodiments, the present disclosure provides methods for reducing risk of a condition, disorder or disease, comprising administering or delivering to a subject susceptible thereto an effective amount of an agent of the present disclosure. In some embodiments, the present disclosure provides methods for reducing risks of a condition, disorder or disease in a population, comprising administering or delivering to a population of subjects susceptible thereto an effective amount of an agent of the present disclosure. In some embodiments, the present disclosure provides methods for treating conditions, disorders or diseases. In some embodiments, the present disclosure provides methods for treating a condition, disorder or disease, comprising administering or delivering to a subject suffering therefrom an effective amount of an agent of the present disclosure. In some embodiments, a symptom is reduced, removed or prevented. In some embodiments, one or more parameters for assessing a condition, disorder or disease are improved. In some embodiments, survival of subjects are extended. As appreciated by those skilled in the art, in some embodiments, prevention, reduced risks, and/or effects of treatment may be assessed through clinical trials and may be observed in subject populations. In some embodiments, a condition, disorder or disease is cancer. In some embodiments, a condition, disorder or disease is associated with beta-catenin. In some embodiments, a condition, disorder or disease is associated with beta-catenin interaction with TCF. In some embodiments, a condition, disorder or disease is bladder cancer. In some embodiments, a condition, disorder or disease is endometrial cancer. In some embodiments, a condition, disorder or disease is adrenocortical carcinoma. In some embodiments, a condition, disorder or disease is gastric cancer. In some embodiments, a condition, disorder or disease is lung cancer. In some embodiments, a condition, disorder or disease is melanoma. In some embodiments, a condition, disorder or disease is esophageal cancer. In some embodiments, a condition, disorder or disease is colorectal cancer. In some embodiments, a cancer is liver cancer. In some embodiments, a cancer is prostate cancer. In some embodiments, a cancer is breast cancer. In some embodiments, a cancer is endometrial cancer. Mutations that lead to constitutive activation of Wnt/beta-catenin-mediated signaling are reported to be present in approximately 20% of all human cancers. In some embodiments, a condition, disorder or disease is associated with WNT signaling. In some embodiments, a condition, disorder or disease is associated with beta-catenin dependent WNT signaling. In some embodiments, a condition, disorder or disease is associated with beta-catenin/TCF interaction. In some embodiments, it has been reported that beta-catenin/TCFs interactions may promote cell proliferation, epithelial-mesenchymal transition (EMT), a cancer stem cell phenotype, etc.

In some embodiments, agents are administered as pharmaceutically compositions that comprise or deliver such agents. In some embodiments, agents are provided and/or delivered in pharmaceutically acceptable salt forms. In some embodiments, in a composition (e.g., a liquid composition of certain pH) an agent may exist in various forms including various pharmaceutically acceptable salt forms.

In some embodiments, a provided agent is utilized in combination with a second therapy. In some embodiments, a provided agent is utilized in combination with a second therapeutic agent. In some embodiments, a second therapy or therapeutic agent is administered prior to an administration or delivery of a provided agent. In some embodiments, a second therapy or therapeutic agent is administered at about the same time as an administration or delivery of a provided agent. In some embodiments, a second therapy or therapeutic agent is administered subsequently to an administration or delivery of a provided agent. In some embodiments, a subject is exposed to both a provided agent and a second therapeutic agent. In some embodiments, a subject is exposed to a therapeutic effect of a provided agent and a therapeutic effect of a second therapeutic agent. In some embodiments, a second therapy is or comprises surgery. In some embodiments, a second therapy is or comprises radiation therapy. In some embodiments, a second therapy is or comprises immunotherapy. In some embodiments, a second therapeutic agent is or comprises a drug. In some embodiments, a second therapeutic agent is or comprises a cancer drug. In some embodiments, a second therapeutic agent is or comprises a chemotherapeutic agent. In some embodiments, a second therapeutic agent is or comprises a hormone therapy agent. In some embodiments, a second therapeutic agent is or comprises a kinase inhibitor. In some embodiments, a second therapeutic agent is or comprises a checkpoint inhibitor (e.g., antibodies against PD-1, PD-L1, CTLA-4, etc.). In some embodiments, a provide agent can be administered with lower unit dose and/or total dose compared to being used alone. In some embodiments, a second agent can be administered with lower unit dose and/or total dose compared to being used alone. In some embodiments, one or more side effects associated with administration of a provided agent and/or a second therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

Further description of certain embodiments of provided technologies is presented below.

### BRIEF DESCRIPTION OF THE DRAWING

FIG. 1. Provided technologies can inhibit beta-catenin driven gene transcription selectively in cells expressing beta-catenin. Stapled peptides inhibited endogenous gene expression in wild HAP1 isogenic cell but not in CTNNB1 knockout (KO) cells. (A): beta-catenin levels. CHIR: CHIR99021, which can activate beta-catenin pathway and increase AXIN2 and SP5 expression. (B): SP5 expression (24h). (C): AXIN2 expression (24h). For each group, from left to right, DMSO (“0” and “0”), Peptide A (1 and 5 uM), I-66 (1 and 5 uM) and I-470 (1 and 5 uM). Expression assessed after 24 hour treatment.

FIG. 2. Provided technologies can reduce nuclear beta-catenin levels. Results for total beta-catenin in nuclear fraction (24 h) are shown as examples.

FIG. 3. Provided technologies can inhibit cell proliferation, modulate transcription and/or induce cell cycle arrest. (A): Provided technologies can reduce cell proliferation. (B) and (C): Provided technologies can modulate gene expression. (B): AXIN 24 hr. (C): CXCL12 24 hr. (D): Provided technologies can induce cell cycle arrest. For left to right: Peptide A (1, 5 and 10 uM), I-66 (1, 5 and 10 uM), I-470 (1, 5 and 10 uM) and DMSO.

FIG. 4. Provided technologies can provide robust, dose-dependent anti-tumor effects in vivo. Both dose levels assessed provided robust reduction of tumor sizes, and the higher dose levels provided greater reductions. COLO320DM cells (colon cancer, mutations: APC, TP53) were utilized for the presented data. Top line is for vehicle treatment, the middle line is for I-66, 30 mg/kg, Q4D, and the bottom line is for I-66, 75 mg/kg, Q4D.

FIG. 5. Provided technologies can provide sustained tumor exposure, suitable pharmacokinetic profiles and broad tissue distribution. (A): Sustained COLO320DM xenograft tumor exposure after a single i.p. injection of I-66 at 50 mg/kg was shown as an example. Dotted line indicates in vitro proliferation IC₅₀ (0.7 uM). (B): Mouse plasma pharmacokinetics. Data presented are I-66 plasma concentration (ng/mL) over time as examples. (C): Tissue distribution observed for I-66 in one assessment. Mouse single dose IP, 50 mg/kg. For each sample, the left column is 24 h data and the right is 96 h data.

FIG. 6. ¹H NMR of a preparation of I-66 prepared as described in Example 9 (DMSO-d6, 373K).

FIG. 7. Integration of peaks in a ¹H NMR spectrum of a preparation of I-66 prepared as described in Example 9 (DMSO-d6, 373K). Those skilled in the art appreciate that integration may be further adjusted and/or optimized.

FIG. 8. Provided technologies can provide robust anti-tumor effects in vivo in multiple tumor models. (A): Certain data from a PDX colon cancer model. (B): Certain data from a PDX CRC model.

### DETAILED DESCRIPTION OF CERTAIN EMBODIMENTS

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand (e.g., an agent) binds to its partner (e.g., beta-catenin or a portion thereof). Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is a stapled peptide.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Amino acid: In its broadest sense, as used herein, refers to any compound and/or substance that can be incorporated into a polypeptide chain, e.g., through formation of one or more peptide bonds. In some embodiments, an amino acid comprising an amino group and an a carboxylic acid group. In some embodiments, an amino acid has the structure of NH(R^{a1})-L^{a1}-C(R^{a2})(R^{a3})-L^{a2}-COOH, wherein each variable is independently as described in the present disclosure. In some embodiments, an amino acid has the general structure NH(R′)—C(R′)₂—COOH, wherein each R′ is independently as described in the present disclosure. In some embodiments, an amino acid has the general structure H₂N—C(R′)₂—COOH, wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population).

Binding: It will be understood that the term “binding”, as used herein, typically refers to a non-covalent association between or among agents. In many embodiments herein, binding is addressed with respect to particular agents and beta-catenin. It will be appreciated by those of ordinary skill in the art that such binding may be assessed in any of a variety of contexts. In some embodiments, binding is assessed with respect to beta-catenin. In some embodiments, binding is assessed with respect to one or more amino acid residues of beta-catenin. In some embodiments, binding is assessed with respect to one or more amino acid residues corresponding to (e.g., similarly positioned in three dimensional space and/or having certain similar properties and/or functions) those of beta-catenin.

Binding site: The term “binding site”, as used herein, refers to a region of a target polypeptide, formed in three-dimensional space, that includes one or more or all interaction residues of the target polypeptide. In some embodiments, “binding site” may refer to one or more amino acid residues which comprise or are one or more or all interaction amino acid residues of a target polypeptide. As will be understood by those of ordinary skill in the art, a binding site may include residues that are adjacent to one another on a linear chain, and/or that are distal to one another on a linear chain but near to one another in three-dimensional space when a target polypeptide is folded. A binding site may comprise amino acid residues and/or saccharide residues.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₁₀, or C₃-C₆ hydrocarbon, or a C₄-C₁₀, or C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, in some embodiments, a peptide may be considered to be engineered if its amino acid sequence has been selected by man. For example, an engineered agent has an amino acid sequence that was selected based on preferences for corresponding amino acids at particular sites of protein-protein interactions. In some embodiments, an engineered sequence has an amino acid sequence that differs from the amino acid sequence of polypeptides included in the NCBI database that binds to a TCF site of beta-catenin. In many embodiments, provided agents are engineered agents. In some embodiments, engineered agents are peptide agents comprising non-natural amino acid residues, non-natural amino acid sequences, and/or peptide staples. In some embodiments, provided agents comprise or are engineered peptide agents which comprise engineered sequences.

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75% 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below (hydrophobicity scale of Kyte and Doolittle, 1982: A simple method for displaying the hydropathic character of a protein. J. Mol. Biol. 157:105-132):

| | | | | Side | Hydropathy |
|---|---|---|---|---|---|
| | | | Side | Chain | Index of |
| | 3 Letter | 1 Letter | Chain | Acidity/ | Kyte and |
| Amino Acid | Code | Code | Polarity | Basicity | Doolittle |
| Alanine | Ala | A | nonpolar | neutral | 1.8 |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | nonpolar | neutral | 2.5 |
| Glutamic acid | Glu | E | polar | acidic | −3.5 |
| Glutamine | Gln | Q | polar | neutral | −3.5 |
| Glycine | Gly | G | nonpolar | neutral | −0.4 |
| Histidine | His | H | polar | basic | −3.2 |
| Isoleucine | Ile | I | nonpolar | neutral | 4.5 |
| Leucine | Leu | L | nonpolar | neutral | 3.8 |
| Lysine | Lys | K | polar | basic | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Interaction residues: The term “interaction residues”, “interaction motifs”, as used herein, refers to, with respect to an agent, residues or motifs in an agent that are designed to interact with particular target residues in a target polypeptide, or with respect to a target polypeptide, residues in a target polypeptide that interact with particular motifs (e.g., aromatic groups, amino acid residues, etc.) of an agent. Specifically, interaction residues and motifs of various agents are selected and arranged within the agents so that they will be displayed in three dimensional space within a predetermined distance (or volume) of identified target residues (e.g., upon binding, docking or other interaction assays). In many embodiments, interaction residues are direct-binding residues.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide” as used herein refers to a polypeptide. In some embodiments, a peptide is a polypeptide that is relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids. In some embodiments, a length is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acids.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; RingeR's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds with bases. Representative alkali or alkaline earth metal salts include salts of sodium, lithium, potassium, calcium, magnesium, and the like. In some embodiments, pharmaceutically acceptable salts are ammonium salts (e.g., —N(R)₃⁺). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In some embodiments, suitable mono-protected amines include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. In some embodiments, suitable di-protected amines include amines that are substituted with two substituents independently selected from those described above as mono-protected amines, and further include cyclic imides, such as phthalimide, maleimide, succinimide, and the like. In some embodiments, suitable di-protected amines include pyrroles and the like, 2,2,5,5-tetramethyl-[1,2,5]azadisilolidine and the like, and azide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl. In some embodiments, suitable protected carboxylic acids include, but are not limited to, optionally substituted C₁-aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifluoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specificity: As is known in the art, “specificity” is a measure of the ability of a particular ligand (e.g., an agent) to distinguish its binding partner (e.g., beta-catenin) from other potential binding partners (e.g., another protein, another portion (e.g., domain) of beta-catenin.

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In some embodiments, example substituents are described below.

Suitable monovalent substituents are halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)N(R^{∘})₂; —N(R^{∘})C(S)N(R^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)N(R^{∘})₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSi(R^{∘})₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)N(R^{∘})₂; —C(S)N(R^{∘})₂; —C(S)SR^{∘}; —SC(S)SR^{∘}, —(CH₂)₀₋₄OC(O)N(R^{∘})₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂N(R^{∘})₂; —(CH₂)₀₋₄S(O)R^{∘}; —N(R^{∘})S(O)₂N(R^{∘})₂; —N(R^{∘})S(O)₂R^{∘}; —N(OR^{∘})R^{∘}; —C(NH)N(R^{∘})₂; —Si(R^{∘})₃; —OSi(R^{∘})₃; —P(R^{∘})₂; —P(OR^{∘})₂; —OP(R^{∘})₂; —OP(OR^{∘})₂; —N(R^{∘})P(R^{∘})₂; —B(R^{∘})₂; —OB(R^{∘})₂; —P(O)(R^{∘})₂; —OP(O)(R^{∘})₂; —N(R^{∘})P(O)(R^{∘})₂; —(C₁₋₄ straight or branched alkylene)O—N(R^{∘})₂; or —(C₁₋₄ straight or branched alkylene)C(O)O—N(R^{∘})₂; wherein each R^{∘} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents are the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R*₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R^{*} are halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each RT is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of RT are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target polypeptide: A “target polypeptide”, as that term is used herein, is a polypeptide with which an agent interacts. In some embodiments, a target polypeptide is a beta-catenin polypeptide. In some embodiments, a target polypeptide comprises, consists essentially of, or is a binding site of beta-catenin polypeptide.

Target residue: A “target residue”, as that term is used herein, is a residue within a target polypeptide with which an agent is designed to interact. For example, an agent may be characterized by particular interaction motifs (e.g., aromatic groups as described herein) and/or residues (e.g., amino acid residues comprising aromatic groups as described herein) selected and arranged (by virtue of being presented on the selected scaffold) to be within a certain predetermined distance (or volume) of a target residue. In some embodiments, a target residue is or comprises an amino acid residue.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Unless otherwise specified, salts, such as pharmaceutically acceptable acid or base addition salts, stereoisomeric forms, and tautomeric forms, of provided compound are included.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

### Stapled Peptides

In some embodiments, a provided agent is or comprises a peptide. In some embodiments, a provided agent is a peptide. In some embodiments, a peptide is a stapled peptide. In some embodiments, a provided agent is a stapled peptide. In some embodiments, a peptide is a stitched peptide. In some embodiments, a provided agent is a stitched peptide. In some embodiments, a stitched peptide comprises two or more staples, wherein two staples are bonded to the same peptide backbone atom. Stapled peptides as described herein are typically peptides in which two or more amino acids of a peptide chain are linked through connection of two peptide backbone atoms of the amino acid residues and, as is understood by those skilled in the art, the connection is not through the peptide backbone between the linked amino acid residues. In some embodiments, a staple as described herein is a linker that link one amino acid residue to another amino acid residue, e.g., through bonding to a peptide backbone atom of each of the amino acid residues and, as is understood by those skilled in the art, the connection through a staple is not through the peptide backbone between the linked amino acid residues. In some embodiments, a staple bonds to the peptide backbone by replacing one or more hydrogen and/or substituents (e.g., side chains, 0, S, etc.) on peptide backbone atoms (e.g., C, N, etc.). In some embodiments, side chains form portions of staples. In some embodiments, a staple is bonded to two carbon backbone atoms, e.g., two alpha carbon atoms. In some embodiments, a staple comprises C(R′)₂ or N(R′), either individually or as part of a large moiety, wherein R′ is R and is taken together with another group attached to a backbone atom which can be R (e.g., R^{a3}) and their intervening atoms to form a ring as described herein (e.g., when PyrS2 is stapled in various peptides).

In some embodiments, a stapled peptide comprises one or more staples. In some embodiments, a stapled peptide comprises two or more staples. In some embodiments, a stapled peptide comprises three or more staples. In some embodiments, a stapled peptide comprises four or more staples. In some embodiments, there are three staples in a stapled peptide. In some embodiments, there are four staples in a stapled peptide.

As will be appreciated by those of ordinary skill in the art, a variety of peptide stapling technologies are available, including both hydrocarbon-stapling and non-hydrocarbon-stapling technologies, and can be utilized in accordance with the present disclosure. Various technologies for stapled and stitched peptides, including various staples and/or methods for manufacturing are available and may be utilized in accordance with the present disclosure, e.g., those described in WO 2019/051327 and WO 2020/041270, the staples of each of which are incorporated herein by reference.

In some embodiments, a peptide, e.g., a stapled peptide, is or comprise a helical structure. In some embodiments, a peptide is a stapled peptide.

In some embodiments, a staple is a hydrocarbon staple. In some embodiments, a staple as described herein is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is or comprises at least one sulfur atom derived from an amino acid residue of a polypeptide. In some embodiments, a non-hydrocarbon staple comprises two sulfur atom derived from two different amino acid residues of a polypeptide. In some embodiments, a non-hydrocarbon staple comprises two sulfur atoms derived from two different cysteine residues of a polypeptide. In some embodiments, a staple is a cysteine staple. In some embodiments, a staple is a non-cysteine staple. In some embodiments, a non-hydrocarbon staple is a carbamate staple and comprises a carbamate moiety (e.g., —N(R′)—C(O)—O—) in its chain. In some embodiments, a non-hydrocarbon staple is an amino staple and comprises an amino group (e.g., —N(R′)—) in its chain. In some embodiments, an amino group in an amino staple, e.g., (—N(R′)—) is not bonded to a carbon atom that additionally forms a double bond with a heteroatom (e.g., —C(═O), —C(═S), —C(═N—R′), etc.) so that it is not part of another nitrogen-containing group such as amide, carbamate, etc. In some embodiments, a non-hydrocarbon staple is an ester staple and comprises an ester moiety (—C(O)—O—) in its chain. In some embodiments, a non-hydrocarbon staple is an amide staple and comprises an amide moiety (—C(O)—N(R′)—) in its chain. In some embodiments, a non-hydrocarbon staple is a sulfonamide staple and comprises a sulfonamide moiety (—S(O)₂—N(R′)—) in its chain. In some embodiments, a non-hydrocarbon staple is an ether staple and comprises an ether moiety (—O—) in its chain. In some embodiments, R′ of a carbamate moiety, amino group, amide moiety, sulfonamide moiety, or ether moiety is R, and is taken together with an R group attached to a backbone (e.g., R^{a3} when it is R) and their intervening atoms to form a ring as described herein. In some embodiments, R′ of a carbamate moiety or amino group is R, and is taken together with an R group attached to a backbone (e.g., R^{a3} when it is R) and their intervening atoms to form a ring as described herein.

In some embodiments, a staple comprises one or more amino groups, e.g., —N(R′)—, wherein each R′ is independently as described herein. In some embodiments, —N(R′)— bonds to two carbon atoms. In some embodiments, —N(R′)— bonds to two carbon atoms, wherein neither of the two carbon atoms are bond to any heteroatoms through a double bond. In some embodiments, —N(R′)— bonds to two sp3 carbon atoms. In some embodiments, a staple comprises one or more —C(O)—N(R′)— groups, wherein each R′ is independently as described herein. In some embodiments, a staple comprises one or more carbamate groups, e.g., one or more —(O)—C(O)—N(R′)—, wherein each R′ is independently as described herein. In some embodiments, R′ is —H. In some embodiments, R′ is optionally substituted C₁₋₆ aliphatic. In some embodiments, R′ is optionally substituted C₁₋₆ alkyl. In some embodiments, R′ is C₁₋₆ aliphatic. In some embodiments, R′ is C₁₋₆ alkyl. In some embodiments, R′ is methyl.

In some embodiments, a stapled peptide comprise one or more staples. In some embodiments, a stapled peptide comprises one and no more than one staple. In some embodiments, a stapled peptide comprises two and no more than two staples. In some embodiments, two staples of a stapled peptide bond to a common backbone atom. In some embodiments, two staples of a stapled peptide bond to a common backbone atom which is an alpha carbon atom of an amino acid residue. In some embodiments, a stapled peptide comprises three or more staples. In some embodiments, a stapled peptides comprise four or more staples. In some embodiments, a stapled peptide comprises three and no more than three staples. In some embodiments, a stapled peptide comprises four and no more than four staples. In some embodiments, each staple independently has the structure of -L^{s1}-L^{s2}-L^{s3}- as described herein. In some embodiments, each staple is independently bonded to two amino acid residues. In some embodiments, each staple is independently bonded to two alpha carbon atoms.

In some embodiments, two, three, four, or all staples of a stapled peptide are within a region that has a length of several amino acid residues. In some embodiments, two staples are within such a region. In some embodiments, three staples are within such a region. In some embodiments, four staples are within such a region. In some embodiments, all staples are within such a region. In some embodiments, a region has a length of 5-20, 5-15, 5-14, 5-113, 5-12, 5-11, 5-10, 6-20, 6-15, 6-14, 6-113, 6-12, 6-11, 6-10, 7-20, 7-15, 7-14, 7-113, 7-12, 7-11, 7-10, 10-16, 10-15, 10-14, 11-16, 11-15, 11-14, 12-16, 12-15, 12-14, 13-15 or 13-14 amino acid residues. In some embodiments, a region has a length of 5 amino acid residues. In some embodiments, a region has a length of 6 amino acid residues. In some embodiments, a region has a length of 7 amino acid residues. In some embodiments, a region has a length of 8 amino acid residues. In some embodiments, a region has a length of 9 amino acid residues. In some embodiments, a region has a length of 10 amino acid residues. In some embodiments, a region has a length of 11 amino acid residues. In some embodiments, a region has a length of 12 amino acid residues. In some embodiments, a region has a length of 13 amino acid residues. In some embodiments, a region has a length of 14 amino acid residues. In some embodiments, a region has a length of 15 amino acid residues. In some embodiments, a region has a length of 16 amino acid residues. In some embodiments, a region has a length of 17 amino acid residues. In some embodiments, a region has a length of 18 amino acid residues. In some embodiments, a region has a length of 19 amino acid residues. In some embodiments, a region has a length of 20 amino acid residues. For example, in various embodiments, stapled peptides comprise three staples within in a region of 14 amino acids (e.g., a staple bonded to aa1 and aa4, a staple bonded to aa4 and aa11, and a staple bonded to aa10 and aa14).

In some embodiments, peptides, e.g., staple peptides, of the present disclosure is or comprises a helix structure. As those skilled in the art will appreciate, helixes can have various lengths. In some embodiments, lengths of helixes range from 5 to 30 amino acid residues. In some embodiments, a length of a helix is 5, 6, 7, 8, 9, 10, 11, 12, 13, 14, 15, 16, 17, 18, 19, or 20, or more, amino acid residues. In some embodiments, a length of a helix is 6 amino acid residues. In some embodiments, a length of a helix is 8 amino acid residues. In some embodiments, a length of a helix is 10 amino acid residues. In some embodiments, a length of a helix is 12 amino acid residues. In some embodiments, a length of a helix is 14 amino acid residues. In some embodiments, a length of a helix is 16 amino acid residues. In some embodiments, a length of a helix is 17 amino acid residues. In some embodiments, a length of a helix is 18 amino acid residues. In some embodiments, a length of a helix is 19 amino acid residues. In some embodiments, a length of a helix is 20 amino acid residues.

Amino acids stapled together can have various number of amino acid residues in between, e.g., 1-20, 1-15, 1-10, 1, 2, 3, 4, 5, 6, 7, 8, 9, or 10, etc. In some embodiments, a staple is (i, i+4) which means there are three amino acid residues between the two amino acids (at positions i and i+4, respectively) that bond to the staple (at positions i+1, i+2, i+3, respectively). In some embodiments, a staple is (i, i+2). In some embodiments, a staple is (i, i+3). In some embodiments, a staple is (i, i+5). In some embodiments, a staple is (i, i+6). In some embodiments, a staple is (i, i+7). In some embodiments, a staple is (i, i+8). In some embodiments, a stapled peptide comprises two staples, one is (i, i+2) and the other is (i, i+7). In some embodiments, a stapled peptide comprises two staples, one is (i, i+3) and the other is (i, i+7). In some embodiments, a stapled peptide comprises two staples, one is (i, i+3) and the other is (i, i+4). In some embodiments, a stapled peptide comprises two staples, one is (i, i+4) and the other is (i, i+7). In some embodiments, a stapled peptide comprises two staples, one is (i, i+3) and the other is (i, i+3). In some embodiments, a stapled peptide comprises two staples, one is (i, i+4) and the other is (i, i+4). In some embodiments, a stapled peptide comprises two staples, one is (i, i+7) and the other is (i, i+7). In some embodiments, the two staples are bonded to a common backbone atom, e.g., an alpha carbon atom of an amino acid residue. In some embodiments, a stapled peptide further comprises a third staple. In some embodiments, a third staple is (i, i+3). In some embodiments, a third staple is (i, i+4). In some embodiments, a third staple is (i, i+7). In some embodiments, a stapled peptide further comprises a fourth staple. In some embodiments, a fourth staple is (i, i+3). In some embodiments, a fourth staple is (i, i+4). In some embodiments, a fourth staple is (i, i+7).

In some embodiments, a stapled peptide comprises a staple which staple is L^{s}, wherein L^{s} is -L^{s1}-L^{s2}-L^{s3}-, each of L^{s1}, L^{s2}, and L^{s3} is independently L, wherein each L is independently as described in the present disclosure. In some embodiments, a provided staple is L^{s}.

In some embodiments, L^{s1} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s1} is -L′-N(R′)—, wherein L′ is optionally substituted bivalent C₁-C₁₉ aliphatic. In some embodiments, L^{s1} is -L′-N(CH₃)—, wherein L′ is optionally substituted bivalent C₁-C₁₉ aliphatic.

In some embodiments, R′ is optionally substituted C₁₋₆ alkyl. In some embodiments, R′ is C₁₋₆ alkyl. In some embodiments, R′ is methyl. In some embodiments, the peptide backbone atom to which L^{s1} is bonded is also bonded to R′, and R′ and R¹ are both R and are taken together with their intervene atoms to form an optionally substituted ring as described in the present disclosure. In some embodiments, a formed ring has no additional ring heteroatoms in addition to the nitrogen atom to which R′ is bonded. In some embodiments, a formed ring is 3-membered. In some embodiments, a formed ring is 4-membered. In some embodiments, a formed ring is 5-membered. In some embodiments, a formed ring is 6-membered.

In some embodiments, L′ is optionally substituted bivalent C₁-C₂₀ aliphatic. In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₈ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₄ aliphatic. In some embodiments, L′ is optionally substituted alkylene. In some embodiments, L′ is optionally substituted alkenylene. In some embodiments, L′ is unsubstituted alkylene. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—. In some embodiments, L′ is bonded to a peptide backbone atom. In some embodiments, L′ is optionally substituted alkenylene. In some embodiments, L′ is unsubstituted alkenylene. In some embodiments, L′ is —CH₂—CH═CH—CH₂—.

In some embodiments, L′ is optionally substituted phenylene.

In some embodiments, L^{s1} comprises at least one —N(R′)C(O)—, wherein R′ is as described in the present disclosure. In some embodiments, L^{s1} is -L′-N(R′)C(O)—, wherein each of L′ and R′ is independently as described in the present disclosure. In some embodiments, L^{s1} is -L′-N(CH₃)C(O)—, wherein L′ is independently as described in the present disclosure.

In some embodiments, L^{s1} comprises at least one —C(O)O—. In some embodiments, L^{s1} comprises at least one —C(O)O—. In some embodiments, L^{s1} is -L′—C(O)O— or -L′-OC(O)—, wherein each L′ is independently as described in the present disclosure. In some embodiments, L^{s1} is -L′—C(O)O—, wherein each L′ is independently as described in the present disclosure. In some embodiments, L^{s1} is -L′-OC(O)—, wherein each L′ is independently as described in the present disclosure.

In some embodiments, L^{s1} comprises at least one —S(O)₂—N(R′)—, wherein R′ is as described in the present disclosure. In some embodiments, L^{s1} comprises at least one —S(O)₂—N(R′)—, wherein R′ is as described in the present disclosure. In some embodiments, L^{s1} is -L′-N(R′)—S(O)₂— or -L′-S(O)₂—N(R′)—, wherein each of L′ and R′ is independently as described in the present disclosure. In some embodiments, L^{s1} is -L′-N(R′)—S(O)₂—, wherein each of L′ and R′ is independently as described in the present disclosure. In some embodiments, L^{s1} is -L′-S(O)₂—N(R′)—, wherein each of L′ and R′ is independently as described in the present disclosure. In some embodiments, L^{s1} is -L′-N(CH₃)—S(O)₂— or -L′-S(O)₂—N(CH₃)—, wherein each L′ is independently as described in the present disclosure. In some embodiments, L^{s1} is -L′-N(CH₃)—S(O)₂—, wherein L′ is as described in the present disclosure. In some embodiments, L^{s1} is -L′-S(O)₂—N(CH₃)—, wherein L′ is as described in the present disclosure.

In some embodiments, L^{s1} comprises at least one —O—. In some embodiments, L^{s1} is -L′-O—, wherein L′ is independently as described in the present disclosure.

In some embodiments, L^{s1} is a covalent bond.

In some embodiments, L^{s1} is L′, wherein L′ is as described in the present disclosure.

In some embodiments, L^{s2} is L, wherein L is as described in the present disclosure. In some embodiments, L^{s2} is L′, wherein L′ is as described in the present disclosure. In some embodiments, L^{s2} comprises —CH₂—CH═CH—CH₂—. In some embodiments, L^{s2} is —CH₂—CH═CH—CH₂—. In some embodiments, L^{s2} comprises —(CH₂)₄—. In some embodiments, L^{s2} is —(CH₂)₄—.

In some embodiments, L^{s3} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s3} is -L′-N(R′)—, wherein L′ is optionally substituted bivalent C₁-C₁₉ aliphatic. In some embodiments, L^{s3} is -L′-N(CH₃)—, wherein L′ is optionally substituted bivalent C₁-C₁₉ aliphatic.

In some embodiments, L^{s3} comprises at least one —N(R′)C(O)—, wherein R′ is as described in the present disclosure. In some embodiments, L^{s3} is -L′-N(R′)C(O)—, wherein each of L′ and R′ is independently as described in the present disclosure. In some embodiments, L^{s3} is -L′-N(CH₃)C(O)—, wherein L′ is independently as described in the present disclosure.

In some embodiments, L^{s3} comprises at least one —C(O)O—. In some embodiments, L^{s3} comprises at least one —C(O)O—. In some embodiments, L^{s3} is -L′—C(O)O— or -L′-OC(O)—, wherein each L′ is independently as described in the present disclosure. In some embodiments, L^{s3} is -L′—C(O)O—, wherein each L′ is independently as described in the present disclosure. In some embodiments, L^{s3} is -L′-OC(O)—, wherein each L′ is independently as described in the present disclosure.

In some embodiments, L^{s3} comprises at least one —S(O)₂—N(R′)—, wherein R′ is as described in the present disclosure. In some embodiments, L^{s3} comprises at least one —S(O)₂—N(R′)—, wherein R′ is as described in the present disclosure. In some embodiments, L^{s3} is -L′-N(R′)—S(O)₂— or -L′-S(O)₂—N(R′)—, wherein each of L′ and R′ is independently as described in the present disclosure. In some embodiments, L^{s3} is -L′-N(R′)—S(O)₂—, wherein each of L′ and R′ is independently as described in the present disclosure. In some embodiments, L^{s3} is -L′-S(O)₂—N(R′)—, wherein each of L′ and R′ is independently as described in the present disclosure. In some embodiments, L^{s3} is -L′-N(CH₃)—S(O)₂— or -L′-S(O)₂—N(CH₃)—, wherein each L′ is independently as described in the present disclosure. In some embodiments, L^{s3} is -L′-N(CH₃)—S(O)₂—, wherein L′ is as described in the present disclosure. In some embodiments, L^{s3} is -L′-S(O)₂—N(CH₃)—, wherein L′ is as described in the present disclosure.

In some embodiments, L^{s3} comprises at least one —O—. In some embodiments, L^{s3} is -L′-O—, wherein L′ is independently as described in the present disclosure.

In some embodiments, L^{s3} is L′, wherein L′ is as described in the present disclosure. In some embodiments, L^{s3} is optionally substituted alkylene. In some embodiments, L^{s3} is unsubstituted alkylene.

In some embodiments, L^{s} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s} comprises at least one —N(R′)C(O)—, wherein R′ is as described in the present disclosure.

In some embodiments, L^{s}, L^{s1}, L^{s2}, and L^{s3} each independently and optionally comprise a R′ group, e.g., a R′ group in —C(R′)₂—, —N(R′)—, etc., and the R′ group is taken with a group (e.g., a group that can be R) attached to a backbone atom (e.g., R^{a1}, R^{a2}, R^{a3}, a R′ group of L^{a1} or L^{a2} (e.g., a R′ group in —C(R′)₂—, —N(R′)—, etc.), etc.) to form a double bond or an optionally substituted ring as two R groups can. In some embodiments, a formed ring is an optionally substituted 3-10 membered ring. In some embodiments, a formed ring is an optionally substituted 3-membered ring. In some embodiments, a formed ring is an optionally substituted 4-membered ring. In some embodiments, a formed ring is an optionally substituted 5-membered ring. In some embodiments, a formed ring is an optionally substituted 6-membered ring. In some embodiments, a formed ring is monocyclic. In some embodiments, a formed ring is saturated. In some embodiments, a formed ring is partially unsaturated. In some embodiments, a formed ring is aromatic. In some embodiments, a formed ring comprises one or more ring heteroatom (e.g., nitrogen). In some embodiments, a staple, or L^{s}, L^{s1}, L^{s2}, and/or L^{s3} comprises —N(R′)—, and the R′ is taken together with a group attached to a backbone atom to form an optionally substituted ring as described herein. In some embodiments, a staple, or L^{s}, L^{s1}, L^{s2}, and/or L^{s3} comprises —C(R′)₂—, and the R′ is taken together with a group attached to a backbone atom to form an optionally substituted ring as described herein.

In some embodiments, a staple, or L^{s}, L^{s1}, L^{s2}, and/or L^{s3} comprises portions of one or more amino acid side chains (e.g., a side chain other than its terminal ═CH₂).

As will be clear to those skilled in the art reading the present disclosure, the letter “L” is used to refer to a linker moiety as described herein; each L^{superscript} (e.g., L^{a}, L^{s1}, L^{s2}, L^{s3}, L^{s}, etc.) therefore is understood, in some embodiments, to be L, unless otherwise specified.

In some embodiments, L comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L is -L′-N(R′)—, wherein L′ is optionally substituted bivalent C₁-C₁₉ aliphatic. In some embodiments, L is -L′-N(CH₃)—, wherein L′ is optionally substituted bivalent C₁-C₁₉ aliphatic.

In some embodiments, L comprises at least one —N(R′)C(O)—, wherein R′ is as described in the present disclosure. In some embodiments, L is -L′-N(R′)C(O)—, wherein each of L′ and R′ is independently as described in the present disclosure. In some embodiments, L is -L′-N(CH₃)C(O)—, wherein L′ is independently as described in the present disclosure.

In some embodiments, L comprises at least one —C(O)O—. In some embodiments, L comprises at least one —C(O)O—. In some embodiments, L is -L′—C(O)O— or -L′-OC(O)—, wherein each L′ is independently as described in the present disclosure. In some embodiments, L is -L′—C(O)O—, wherein each L′ is independently as described in the present disclosure. In some embodiments, L is -L′-OC(O)—, wherein each L′ is independently as described in the present disclosure.

In some embodiments, L comprises at least one —S(O)₂—N(R′)—, wherein R′ is as described in the present disclosure. In some embodiments, L comprises at least one —S(O)₂—N(R′)—, wherein R′ is as described in the present disclosure. In some embodiments, L is -L′-N(R′)—S(O)₂— or -L′-S(O)₂—N(R′)—, wherein each of L′ and R′ is independently as described in the present disclosure. In some embodiments, L is -L′-N(R′)—S(O)₂—, wherein each of L′ and R′ is independently as described in the present disclosure. In some embodiments, L is -L′-S(O)₂—N(R′)—, wherein each of L′ and R′ is independently as described in the present disclosure. In some embodiments, L is -L′-N(CH₃)—S(O)₂— or -L′-S(O)₂—N(CH₃)—, wherein each L′ is independently as described in the present disclosure. In some embodiments, L is -L′-N(CH₃)—S(O)₂—, wherein L′ is as described in the present disclosure. In some embodiments, L is -L′-S(O)₂—N(CH₃)—, wherein L′ is as described in the present disclosure.

In some embodiments, L comprises at least one —O—. In some embodiments, L is -L′-O—, wherein L′ is independently as described in the present disclosure.

In some embodiments, L is L′, wherein L′ is as described in the present disclosure. In some embodiments, L is optionally substituted alkylene. In some embodiments, L is unsubstituted alkylene.

In some embodiments, L is optionally substituted bivalent C₁-C₂₅ aliphatic. In some embodiments, L is optionally substituted bivalent C₁-C₂₀ aliphatic. In some embodiments, L is optionally substituted bivalent C₁-C₁₅ aliphatic. In some embodiments, L is optionally substituted bivalent C₁-C₁₀ aliphatic. In some embodiments, L is optionally substituted bivalent C₁-C₉ aliphatic. In some embodiments, L is optionally substituted bivalent C₁-C₅ aliphatic. In some embodiments, L is optionally substituted bivalent C₁-C₇ aliphatic. In some embodiments, L is optionally substituted bivalent C₁-C₆ aliphatic. In some embodiments, L is optionally substituted bivalent C₁-C₅ aliphatic. In some embodiments, L is optionally substituted bivalent C₁-C₄ aliphatic. In some embodiments, L is optionally substituted alkylene. In some embodiments, L is optionally substituted alkenylene. In some embodiments, L is unsubstituted alkylene. In some embodiments, L is —CH₂—. In some embodiments, L is —(CH₂)₂—. In some embodiments, L is —(CH₂)₃—. In some embodiments, L is —(CH₂)₄—. In some embodiments, L is —(CH₂)₅—. In some embodiments, L is —(CH₂)₆—. In some embodiments, L is —(CH₂)₇—. In some embodiments, L is —(CH₂)₈—. In some embodiments, L is bonded to a peptide backbone atom. In some embodiments, L is optionally substituted alkenylene. In some embodiments, L is unsubstituted alkenylene. In some embodiments, L is —CH₂—CH═CH—CH₂—.

In some embodiments, one end of a staple is connected to an atom Aⁿ¹ of the peptide backbone, wherein Aⁿ¹ is optionally substituted with R¹ and is an atom of an amino acid residue at amino acid position n¹ of the peptide from the N-terminus, and the other end is connected to an atom Aⁿ² of the peptide backbone, wherein Aⁿ² is optionally substituted with R² (in some embodiments, R¹ and/or R² is R which can be hydrogen) and is an atom of an amino acid residue at amino acid position n² of the peptide from the N-terminus, wherein each of n¹ and n² is independently an integer, and n²=n¹+m, wherein m is 3-12.

In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, a staple is referred to a (i, i+m) staple.

In some embodiments, Aⁿ¹ is a carbon atom. In some embodiments, Aⁿ¹ is achiral. In some embodiments, Aⁿ¹ is chiral. In some embodiments, Aⁿ¹ is R. In some embodiments, Aⁿ¹ is S.

In some embodiments, Aⁿ² is a carbon atom. In some embodiments, Aⁿ² is achiral. In some embodiments, Aⁿ² is chiral. In some embodiments, Aⁿ² is R. In some embodiments, Aⁿ² is S.

In some embodiments, Aⁿ¹ is achiral and Aⁿ² is achiral. In some embodiments, Aⁿ¹ is achiral and Aⁿ² is R. In some embodiments, Aⁿ¹ is achiral and Aⁿ² is S. In some embodiments, Aⁿ¹ is R and Aⁿ² is achiral. In some embodiments, Aⁿ¹ is R and Aⁿ² is R. In some embodiments, Aⁿ¹ is R and Aⁿ² is S. In some embodiments, Aⁿ¹ is S and Aⁿ² is achiral. In some embodiments, Aⁿ¹ is S and Aⁿ² is R. In some embodiments, Aⁿ¹ is S and Aⁿ² is S.

In some embodiments, provided stereochemistry at staple-backbone connection points and/or combinations thereof, optionally together with one or more structural elements of provided peptide, e.g., staple chemistry (hydrocarbon, non-hydrocarbon), staple length, etc. can provide various benefits, such as improved preparation yield, purity, and/or selectivity, improved properties (e.g., improved solubility, improved stability, lowered toxicity, improved selectivity, etc.), improved activities, etc. In some embodiments, provided stereochemistry and/or stereochemistry combinations are different from those typically used, e.g., those of U.S. Pat. No. 9,617,309, US 2015-0225471, US 2016-0024153, US 2016-0215036, US 2016-0244494, WO 2017/062518, and provided one or more of benefits described in the present disclosure.

In some embodiments, a staple can be of various lengths, in some embodiments, as represent by the number of chain atoms of a staple. In some embodiments, a chain of a staple is the shortest covalent connection in the staple from a first end (connection point with a peptide backbone) of a staple to a second end of the staple, wherein the first end and the second end are connected to two different peptide backbone atoms. In some embodiments, a staple comprises 5-30 chain atoms, e.g., 5-20, 5-15, 5, 6, 7, 8, 9, or 10 to 11, 12, 13, 14, 15, 16, 17, 18, 19, 20, 21, 22, 23, 24, or 25 chain atoms. In some embodiments, a staple comprises 5 chain atoms. In some embodiments, a staple comprises 6 chain atoms. In some embodiments, a staple comprises 7 chain atoms. In some embodiments, a staple comprises 8 chain atoms. In some embodiments, a staple comprises 9 chain atoms. In some embodiments, a staple comprises 10 chain atoms. In some embodiments, a staple comprises 11 chain atoms. In some embodiments, a staple comprises 12 chain atoms. In some embodiments, a staple comprises 13 chain atoms. In some embodiments, a staple comprises 14 chain atoms. In some embodiments, a staple comprises 15 chain atoms. In some embodiments, a staple comprises 16 chain atoms. In some embodiments, a staple comprises 17 chain atoms. In some embodiments, a staple comprises 18 chain atoms. In some embodiments, a staple comprises 19 chain atoms. In some embodiments, a staple comprises 20 chain atoms. In some embodiments, a staple has a length of 5 chain atoms. In some embodiments, a staple has a length of 6 chain atoms. In some embodiments, a staple has a length of 7 chain atoms. In some embodiments, a staple has a length of 8 chain atoms. In some embodiments, a staple has a length of 9 chain atoms. In some embodiments, a staple has a length of 10 chain atoms. In some embodiments, a staple has a length of 11 chain atoms. In some embodiments, a staple has a length of 12 chain atoms. In some embodiments, a staple has a length of 13 chain atoms. In some embodiments, a staple has a length of 14 chain atoms. In some embodiments, a staple has a length of 15 chain atoms. In some embodiments, a staple has a length of 16 chain atoms. In some embodiments, a staple has a length of 17 chain atoms. In some embodiments, a staple has a length of 18 chain atoms. In some embodiments, a staple has a length of 19 chain atoms. In some embodiments, a staple has a length of 20 chain atoms. In some embodiments, a staple has a length of 8-15 chain atoms. In some embodiments, a staple has 8-12 chain atoms. In some embodiments, a staple has 9-12 chain atoms. In some embodiments, a staple has 9-10 chain atoms. In some embodiments, a staple has 8-10 chain atoms. In some embodiments, length of a staple can be adjusted according to the distance of the amino acid residues it connects, for example, a longer staple may be utilized for a (i, i+7) staple than a (i, i+4) or (i, i+3) staple. In some embodiments, a (i, i+2) staple has about 5-10, 5-8, e.g., about 5, 6, 7, 8, 9 or 10 chain atoms. In some embodiments, a (i, i+2) staple has 5 chain atoms. In some embodiments, a (i, i+2) staple has 6 chain atoms. In some embodiments, a (i, i+2) staple has 7 chain atoms. In some embodiments, a (i, i+2) staple has 8 chain atoms. In some embodiments, a (i, i+2) staple has 9 chain atoms. In some embodiments, a (i, i+2) staple has 10 chain atoms. In some embodiments, a (i, i+3) staple has about 5-10, 5-8, e.g., about 5, 6, 7, 8, 9 or 10 chain atoms. In some embodiments, a (i, i+3) staple has 5 chain atoms. In some embodiments, a (i, i+3) staple has 6 chain atoms. In some embodiments, a (i, i+3) staple has 7 chain atoms. In some embodiments, a (i, i+3) staple has 8 chain atoms. In some embodiments, a (i, i+3) staple has 9 chain atoms. In some embodiments, a (i, i+3) staple has 10 chain atoms. In some embodiments, a (i, i+4) staple has about 5-12, 5-10, 7-12, 5-8, e.g., about 5, 6, 7, 8, 9, 10, 11 or 12 chain atoms. In some embodiments, a (i, i+4) staple has 5 chain atoms. In some embodiments, a (i, i+4) staple has 6 chain atoms. In some embodiments, a (i, i+4) staple has 7 chain atoms. In some embodiments, a (i, i+4) staple has 8 chain atoms. In some embodiments, a (i, i+4) staple has 9 chain atoms. In some embodiments, a (i, i+4) staple has 10 chain atoms. In some embodiments, a (i, i+4) staple has 11 chain atoms. In some embodiments, a (i, i+4) staple has 12 chain atoms. In some embodiments, a (i, i+7) staple has about 8-25, 10-25, 10-16, 12-15, e.g., about 8, 9, 10, 11, 12, 13, 14, 15, 16, 17, 18, 19, 20, 21, 22, 23, 24 or 25 chain atoms. In some embodiments, a (i, i+7) staple has 8 chain atoms. In some embodiments, a (i, i+7) staple has 9 chain atoms. In some embodiments, a (i, i+7) staple has 10 chain atoms. In some embodiments, a (i, i+7) staple has 11 chain atoms. In some embodiments, a (i, i+7) staple has 12 chain atoms. In some embodiments, a (i, i+7) staple has 13 chain atoms. In some embodiments, a (i, i+7) staple has 14 chain atoms. In some embodiments, a (i, i+7) staple has 15 chain atoms. In some embodiments, a (i, i+7) staple has 16 chain atoms. In some embodiments, a (i, i+7) staple has 17 chain atoms. In some embodiments, a (i, i+7) staple has 18 chain atoms. In some embodiments, a (i, i+7) staple has 19 chain atoms. In some embodiments, a (i, i+7) staple has 20 chain atoms. In some embodiments, a (i, i+7) staple has 21 chain atoms. In some embodiments, a (i, i+7) staple has 22 chain atoms. In some embodiments, a stapled peptide comprises three or more staples, each of which is independently such a (I, i+2), (i, i+3), (i, i+4) or (i, i+7) staple. In some embodiments, a stapled peptide comprises such a (i, i+2) staple, such a (i, i+4) staple and such a (i, i+7) staple. In some embodiments, a stapled peptide comprises such a (i, i+3) staple, such a (i, i+4) staple and such a (i, i+7) staple. In some embodiments, a stapled peptide comprises such a (i, i+3) staple, such a (i, i+7) staple and such a (i, i+7) staple.

Staple lengths may be otherwise described. For example, in some embodiments, staple lengths may be described as the total number of chain atoms and non-chain ring atoms, where a non-chain ring atom is an atom of the staple which forms a ring with one or more chain atoms but is not a chain atom in that it is not within the shortest covalent connection from a first end of the staple to a second end of the staple. In some embodiments, staples formed using Monomer A (which comprises an azetidine moiety), Monomer B (which comprises a pyrrolidine moiety), and/or Monomer C (which comprises a pyrrolidine moiety), etc., may comprise one or two non-chain ring atoms.

In some embodiments, a staple has no heteroatoms in its chain. In some embodiments, a staple comprises at least one heteroatom in its chain. In some embodiments, a staple comprises at least one nitrogen atom in its chain.

In some embodiments, a staple is L^{s}, wherein L^{s} is an optionally substituted, bivalent C₈₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₉₋₁₃ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₀₋₁₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is a (i, i+2) staple in that not including the two amino acid residues that are directly connected to the staple, there are one amino acid residue between the two amino acid residues that are directly connected to the staple. In some embodiments, a staple is a (i, i+3) staple in that not including the two amino acid residues that are directly connected to the staple, there are two amino acid residues between the two amino acid residues that are directly connected to the staple. In some embodiments, a staple is a (i, i+4) staple in that not including the two amino acid residues that are directly connected to the staple, there are three amino acid residues between the two amino acid residues that are directly connected to the staple. In some embodiments, a staple is a (i, i+7) staple in that not including the two amino acid residues that are directly connected to the staple, there are six amino acid residues between the two amino acid residues that are directly connected to the staple.

In some embodiments, for each of L^{s}, L^{s1}, L^{s2}, and L^{s3}, any replacement of methylene units, if any, is replaced with —N(R′)—, —C(O)—N(R′)—, —N(R′)C(O)O—, —C(O)O—, —S(O)₂N(R′)—, or —O—. In some embodiments, for each of L^{s}, L^{s1}, L^{s2}, and L^{s3}, any replacement of methylene units, if any, is replaced with —N(R′)—, —N(R′)—C(O)—, or —N(R′)C(O)O—. In some embodiments, for each of L^{s}, L^{s1}, L^{s2}, and L^{s3}, any replacement of methylene units, if any, is replaced with —N(R′)— or —N(R′)C(O)O—. In some embodiments, for each of L^{s}, L^{s1}, L^{s2}, and L^{s3}, any replacement of methylene units, if any, is replaced with —N(R′)—. In some embodiments, for each of L^{s}, L^{s1}, L^{s2}, and L^{s3}, any replacement of methylene units, if any, is replaced with —N(R′)C(O)O—.

In some embodiments, a staple comprises a double bond. In some embodiments, a staple comprises a double bond may be formed by olefin metathesis of two olefins. In some embodiments, staples are formed by metathesis reactions, e.g., involving one or more double bonds in amino acid residues as described herein. In some embodiments, a first amino acid residue comprising an olefin (e.g., AA1-CH═CH₂) and a second amino acid residue comprising an olefin (e.g., AA2-CH═CH₂) are stapled (e.g., forming AA1-CH═CH-AA2, wherein AA1 and AA2 are typically linked through one or more amino acid residues). In some embodiments, an olefin, e.g., in a staple, is converted into —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—. In some embodiments, each of the two olefins is independently of a side chain of an amino acid residue. In some embodiments, each olefin is independently a terminal olefin. In some embodiments, each olefin is independently a mono-substituted olefin.

In some embodiments, an amino acid of formula A-I or a salt thereof is a compound having the structure of formula A-IL:

NH(R^{a1})-L^{a1}-C(-L^{a}-CH═CH₂)(R^{a3})-L^{a2}-COOH, A-II

or a salt thereof, wherein each variable is independently as described in the present disclosure. In some embodiments, an amino acid suitable for stapling has the structure of formula A-II or a salt thereof, wherein each variable is independently as described in the present disclosure.

In some embodiments, an amino acid of formula A-II or a salt thereof is a compound having the structure of formula A-II-b:

NH(R^{a1})—C(-L^{a}-CH═CH₂)(R^{a3})—COOH, A-II-b

or a salt thereof, wherein each variable is independently as described in the present disclosure. In some embodiments, an amino acid suitable for stapling has the structure of formula A-II-b or a salt thereof, wherein each variable is independently as described in the present disclosure.

In some embodiments, an amino acid of formula A-I or a salt thereof is a compound having the structure of formula A-III:

N(-L^{a}-CH═CH₂)(R^{a1})-L^{a1}-C(-L^{a}-CH═CH₂)(R^{a3})-L^{a2}-COOH, A-III

or a salt thereof, wherein each variable is independently as described in the present disclosure. In some embodiments, an amino acid suitable for stapling has the structure of formula A-II or a salt thereof, wherein each variable is independently as described in the present disclosure.

In some embodiments, an amino acid of formula A-I or a salt thereof has structure of formula A-IV:

NH(R^{a1})-L^{a1}-C(-L^{a}-COOH)(R^{a3})-L^{a2}-COOH, A-IV

or a salt thereof, wherein each variable is independently as described in the present disclosure. In some embodiments, an amino acid suitable for stapling has the structure of formula A-IV or a salt thereof, wherein each variable is independently as described in the present disclosure.

In some embodiments, an amino acid has structure of formula A-V:

NH(R^{a1})-L^{a1}-C(-L^{a}-R^{SP1})(R^{a3})-L^{a2}-COOH, A-V

or a salt thereof, wherein each variable is independently as described in the present disclosure. In some embodiments, an amino acid suitable for stapling has the structure of formula A-V or a salt thereof, wherein each variable is independently as described in the present disclosure.

In some embodiments, an amino acid for stapling has structure of formula A-VI:

NH(R^{a1})-L^{a1}-C(-L^{a}-R^{SP1})(-L^{a}-R^{SP2})-L^{a2}-COOH, A-VI

or a salt thereof, wherein each variable is independently as described in the present disclosure. In some embodiments, an amino acid suitable for stapling has the structure of formula A-VI or a salt thereof, wherein each variable is independently as described in the present disclosure.

As used herein, each of R^{SP1} and R^{SP2} independently comprises a reactive group. In some embodiments, each of R^{SP1} and R^{SP2} is independently a reactive group. In some embodiments, a reactive group is optionally substituted —CH═CH₂. In some embodiments, a reactive group is —CH═CH₂. In some embodiments, a reactive group is an amino group, e.g., —NHR, wherein R is as described herein. In some embodiments, a reactive group is an acid group. In some embodiments, a reactive group is —COOH or an activated form thereof. In some embodiments, a reactive group is for a cycloaddition reaction (e.g., [3+2], [4+2], etc.), e.g., an alkene, an alkyne, a diene, a 1,3-dipole (e.g., —N₃), etc. In some embodiments, a reactive group is optionally substituted —C≡CH. In some embodiments, a reactive group is —C≡CH. In some embodiments, a reactive group is —N₃.

In some embodiments, R^{SP1} or R^{SP2} of a first amino acid residue and R^{SP1} or R^{SP2} of a second amino acid residue can react with each other so that the two amino acid residues are connected with a staple. In some embodiments, a reactive is olefin metathesis between two olefin, e.g., two —CH═CH₂. In some embodiments, a reaction is amidation and one reactive group is an amino group, e.g., —NHR wherein R is as described herein (e.g., in some embodiments, R is —H; in some embodiments, R is optionally substituted C₁₋₆ aliphatic), and the other is an acid group (e.g., —COOH) or an activated form thereof. In some embodiments, a reaction is a cycloaddition reaction, e.g., [4+2], [3+2], etc. In some embodiments, a first and a second reactive groups are two reactive groups suitable for a cycloaddition reaction. In some embodiments, a reaction is a click reaction. In some embodiments, one reaction group is or comprises —N₃, and the other is or comprises an alkyne, e.g., a terminal alkyne or a activated/strained alkyne. In some embodiments, the other is or comprises —C≡CH.

In some embodiments, R^{SP1} or R^{SP2} of a first amino acid residue and R^{SP1} or R^{SP2} of a second amino acid residue can react with a reagent so that the two are connected to form a staple. In some embodiments, a reagent comprises two reactive groups, one of which reacts with R^{SP1} or R^{SP2} of a first amino acid residue, and the other reacts with R^{SP1} or R^{SP2} of a first amino acid residue. In some embodiments, R^{SP1} or R^{SP2} of both amino acid residues are the same or the same type, e.g., both are amino groups, and the two reactive groups of a linking reagent are also the same, e.g., both are acid groups such as —COOH or activated form thereof. In some embodiments, R^{SP1} or R^{SP2} of both amino acid residues are both acid groups, e.g., —COOH or activated form thereof, and both reactive groups of a linking agent are amino groups. In some embodiments, R^{SP1} or R^{SP2} of both amino acid residues are both nucleophilic groups, e.g., —SH, and both reactive groups of a linking reagent are electrophilic (e.g., carbon attached to leaving groups such as —Br, —I, etc.).

In some embodiments, R^{SP1} and R^{SP2} are the same. In some embodiments, R^{SP1} and R^{SP2} are different. In some embodiments, R^{SP1} is or comprises —CH═CH₂. In some embodiments, R^{SP1} is or comprises —COOH. In some embodiments, R^{SP1} is or comprises an amino group. In some embodiments, R^{SP1} is or comprises —NHR. In some embodiments, R is hydrogen or optionally substituted C₁₋₆ aliphatic. In some embodiments, R^{SP1} is or comprises —NH₂. In some embodiments, R^{SP1} is or comprises —N₃. In some embodiments, R^{SP2} is or comprises —CH═CH₂. In some embodiments, R^{SP2} is or comprises —COOH. In some embodiments, R^{SP2} is or comprises an amino group. In some embodiments, R^{SP2} is or comprises —NHR. In some embodiments, R is hydrogen or optionally substituted C₁₋₆ aliphatic. In some embodiments, R^{SP2} is or comprises —NH₂. In some embodiments, R^{SP2} is or comprises —N₃.

In some embodiments, each amino acid residue of a pair of amino acid residues is independently a residue of an amino acid of formula A-II or A-III or a salt thereof. In some embodiments, such a pair of amino acid residues is stapled, e.g., through olefin metathesis. In some embodiments, a staple has the structure of -L^{a}-CH═CH-L^{a}-, wherein each variable is independently as described herein. In some embodiments, olefin in a staple is reduced. In some embodiments, In some embodiments, a staple has the structure of -L^{a}-CH₂—CH₂-L^{a}-, wherein each variable is independently as described herein. In some embodiments, one L^{a} is L^{s1} as described herein, and one L^{a} is L^{s3} as described herein.

In some embodiments, two amino acid residues, e.g., of amino acids independently of formula A-I or a salt of, connected by a staple have the structure of —N(R^{a1})-L^{a1}-C(-L^{s}-R^{AA})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of —N(-L^{s}-R^{AA})-L^{a1}-C(R^{a2})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of R^{a1}—N(-L^{s}-R^{AA})-L^{a1}-C(R^{a2})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of R^{a1}—N(-L^{s}-R^{AA})-L^{a1}-C(-L^{s}-R^{AA})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of —N(-L^{s}-R^{AA})-L^{a1}-C(-L^{s}-R^{AA})(R³)-L^{a2}-CO—, wherein each variable is independently as described herein, and R^{AA} is an amino acid residue. In some embodiments, three amino acid residues, e.g., of amino acids independently of formula A-I or a salt of, connected by two staples (e.g., X⁴ stapled with both X¹ and X¹⁴) have the structure of —N(R^{a1})-L^{a1}-C(-L^{s}-R^{AA})(-L^{s}-R^{AA})-L^{a2}-CO—, wherein each variable is independently as described herein, and R^{AA} is an amino acid residue. In some embodiments, each R^{AA} is independently a residue of an amino acid of formula A-I, A-II, A-III, A-IV, A-V, A-VI, etc. or a salt thereof. In some embodiments, R^{AA} is —C(R^{a3})[-L^{a1}-N(R^{a1})-](-L^{a2}-CO—), wherein each variable is independently as described herein. In some embodiments, R^{AA} is —C(R^{a3})[—N(R^{a1})—](—CO—), wherein each variable is independently as described herein. In some embodiments, each R^{AA} is independently —N(−)[-L^{a1}-C(R^{a2})(R^{a3})-L^{a2}-CO—], wherein each variable is independently as described herein, wherein —C(−)(R^{a3})— is bonded to a staple. In some embodiments, each R^{AA} is independently —N(−)[—C(R^{a2})(R^{a3})CO—], wherein each variable is independently as described herein, wherein —C(−)(R^{a3})— is bonded to a staple. In some embodiments, each R^{AA} is independently R^{a1}—N(−)[-L^{a1}-C(R^{a2})(R^{a3})-L^{a2}-CO—], wherein each variable is independently as described herein, wherein —C(−)(R^{a3})— is bonded to a staple. In some embodiments, each R^{AA} is independently R^{a1}—N(−)[—C(R^{a2})(R^{a3})—CO—], wherein each variable is independently as described herein, wherein —C(−)(R^{a3})— is bonded to a staple.

Various staples, e.g., L^{s}, are as described herein. In some embodiments, L^{s} is -L^{s1}-L^{s2}-L^{s3}- as described herein. In some embodiments, L^{s1} is L^{a} as described herein. In some embodiments, L^{s3} is L^{a} as described herein. In some embodiments, L^{s1} is L^{a} of a first of two stapled amino acid residues. In some embodiments, L^{s2} is L^{a} of a second of two stapled amino acid residues. In some embodiments, L^{s2} is or comprises a double bond. In some embodiments, L^{s2} is or comprises —CH═CH—. In some embodiments, L^{s2} is or comprises optionally substituted —CH₂—CH₂—. In some embodiments, L^{s2} is or comprises —CH₂—CH₂—. In some embodiments, L^{s2} is or comprises —C(O)N(R′)— (e.g., a staple formed by two amino acid residues one of which has a R^{SP1} group that is or comprises an amino group and the other of which has a R^{SP2} group that is or comprises —COOH). In some embodiments, L^{s2} is or comprises —C(O)NH—. In some embodiments, each of L^{s1} and L^{s3} is independently optionally substituted linear or branched C₁₋₁₀ hydrocarbon chain. In some embodiments, each of L^{s1} and L^{s3} is independently —(CH₂)n-, wherein n is 1-10. In some embodiments, L^{s1} is —CH₂—. In some embodiments, L^{s3} is —(CH₂)₃—.

In some embodiments, L^{s} is —CH₂—CH═CH—(CH₂)₃—. In some embodiments, L^{s} is —(CH₂)₆—.

In some embodiments, L^{s} is —(CH₂)₂—C(O)NH—(CH₂)₄—.

In some embodiments, L^{s} is bonded to two backbone carbon atoms. In some embodiments, L^{s} is bonded to two alpha carbon atoms of two stapled amino acid residues. In some embodiments, L^{s} is bonded to a backbone nitrogen atom and a backbone carbon atom (e.g., an alpha carbon).

In some embodiments, L^{a} comprises at least one —N(R′)— wherein R′ is independently as described in the present disclosure. In some embodiments, L^{a} comprises -L^{am1}-N(R′)— wherein R′ is independently as described in the present disclosure, and L^{am1} is as described herein. In some embodiments, L^{a} is or comprises -L^{am1}-N(R′)-L^{am2}-, wherein each of L^{am1}, R′, and L^{am2} is independently as described herein. In some embodiments, R′ is optionally substituted C₁₋₆ aliphatic. In some embodiments, R′ is methyl. In some embodiments, R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

In some embodiments, L^{a} comprises at least one —C(R′)₂— wherein each R′ is independently as described in the present disclosure. In some embodiments, L^{a} comprises -L^{am1}-C(R′)₂— wherein R′ is independently as described in the present disclosure, and L^{am1} is as described herein. In some embodiments, L^{a} is or comprises -L^{am1}-C(R′)₂-L^{am2}-, wherein each of L^{am1}, R′, and L^{am2} is independently as described herein. In some embodiments, R′ is —H. In some embodiments, —C(R′)₂— is optionally substituted —CH₂—. In some embodiments, —C(R′)₂— is —CH₂—. In some embodiments, one R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

As described herein, each of L^{am1} and L^{am2} is independently L^{am} as described herein. As described herein, L^{am} is a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{am} is a covalent bond. In some embodiments, L^{am} is an optionally substituted bivalent C₁-C₁₀ aliphatic group. In some embodiments, L^{a1} is an optionally substituted bivalent linear C₁-C₁₀ aliphatic group. In some embodiments, L^{am} is optionally substituted C₁₋₁₀ alkylene. In some embodiments, L^{am} is C₁₋₁₀ alkylene. In some embodiments, L^{am} is optionally substituted linear C₁₋₁₀ alkylene. In some embodiments, L^{am} is optionally substituted —CH₂—. In some embodiments, L^{am} is —CH₂—.

In some embodiments, L^{am} is a covalent bond. In some embodiments, L^{am1}i is an optionally substituted bivalent C₁-C₁₀ aliphatic group. In some embodiments, L^{am1}i is an optionally substituted bivalent linear C₁-C₁₀ aliphatic group. In some embodiments, L^{am1}i is optionally substituted C₁₋₁₀ alkylene. In some embodiments, L^{am1}i is C₁₀ alkylene. In some embodiments, L^{am1}i is optionally substituted linear C₁₋₁₀ alkylene. In some embodiments, L^{am1}i is optionally substituted —CH₂—. In some embodiments, L^{am} is —CH₂—. In some embodiments, L^{am1}i is bonded to a backbone atom. In some embodiments, L^{am} is bonded to an alpha-carbon of an amino acid.

In some embodiments, L^{am2} is a covalent bond. In some embodiments, L^{am2} is an optionally substituted bivalent C₁-C₁₀ aliphatic group. In some embodiments, L^{am2} is an optionally substituted bivalent linear C₁-C₁₀ aliphatic group. In some embodiments, L^{am2} is optionally substituted C₁₋₁₀ alkylene. In some embodiments, L^{am2} is C₁₋₁₀ alkylene. In some embodiments, L^{am2} is optionally substituted linear C₁₋₁₀ alkylene. In some embodiments, L^{am2} is optionally substituted —CH₂—. In some embodiments, L^{am2} is —CH₂—. In some embodiments, L^{am2} is or comprises —C(O)—. In some embodiments, —C(O)— is bonded to a nitrogen atom. In some embodiments, L^{am2} is or comprises —S(O)₂—. In some embodiments, —S(O)₂— is bonded to a nitrogen atom. In some embodiments, L^{am2} is or comprises —O—. In some embodiments, L^{am2} is or comprises —C(O)—O—. In some embodiments, —C(O)—O— is bonded to a nitrogen atom. In some embodiments, L^{am2} is bonded to a nitrogen atom, and it comprises a —C(O)— group which is bonded to the nitrogen atom. In some embodiments, L^{am2} is bonded to a nitrogen atom, and it comprises a —C(O)—O— group which is bonded to the nitrogen atom. In some embodiments, L^{am2} is or comprises —C(O)—O—CH₂—, wherein the —CH₂— is optionally substituted. In some embodiments, L^{am2} is —C(O)—O—CH₂—.

In some embodiments, L^{a} is L^{s1} as described herein. In some embodiments, L^{a} is L^{s2} as described herein.

In some embodiments, R^{a3} is -L^{a}-CH═CH₂, wherein L^{a} is independently as described herein. In some embodiments, each of R^{a2} and R^{a3} independently comprises a double bond, e.g., a terminal olefin which can be optionally and independently stapled with another residue comprising an olefin. In some embodiments, each of R^{a2} and R^{a3} are independently -L^{a}-CH═CH₂. In some embodiments, an amino acid are stapled with two amino acid residues independently through R^{a2} and R^{a3}. In some embodiments, such an amino acid is B5. In some embodiments, it is B3. In some embodiments, it is B4. In some embodiments, it is B6.

In some embodiments, an amino acid is selected from Tables A-I, A-II, A-III and A-IV (may be presented as Fmoc-protected). As appreciated by those skilled in the art, among other things, when incorporated into peptides, Fmoc-protected amino groups and carboxyl groups may independently form amide connections with other amino acid residues (or N- or C-terminus capping groups, or exist as N- or C-terminus amino or carboxyl groups). Olefins, including those in Alloc groups, may be utilized to form staples through olefin metathesis. Staples comprising olefins may be further modified, e.g., through hydrogenation to convert olefin double bonds into single bonds, and/or through CO₂ extrusion to convert carbamate moieties (e.g., —O—(CO)—N(R′)—) into amine moieties (e.g., —N(R′)—). In some embodiments, an agent is or comprises a stapled peptide (e.g., a stapled peptide described according to Table E2 or Table E3) or a salt thereof, in which stapled peptide each double bond is converted into a single bond. In some embodiments, a conversion is achieved through hydrogenation which adds a —H to each olefin carbon atom. In some embodiments, an olefin double bond is replaced with —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—.

TABLE A-I
Exemplary amino acids (Fmoc-Protected).
Monomer A (M_{A})

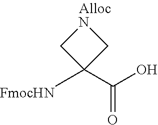

Monomer B (M_{B})

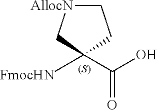

Monomer C (M_{C})

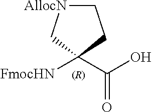

TABLE A-II
Exemplary amino acids (Fmoc-Protected).
Monomer D (M_{D})

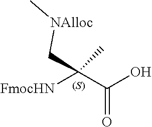

Monomer E (M_{E})

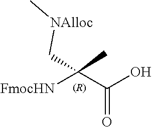

Monomer F (M_{F})

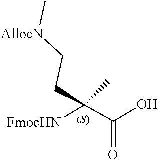

Monomer G (M_{G})

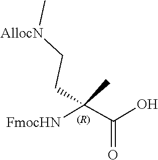

Monomer H (M_{H})

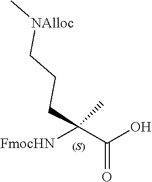

Monomer I (M_{I})

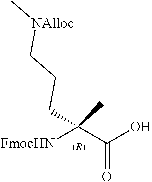

TABLE A-III
Exemplary amino acids (Fmoc-Protected).
S3

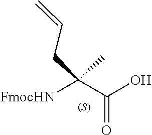

R3

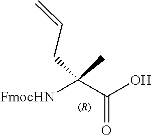

S4

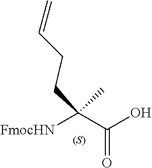

R4

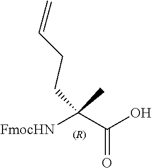

S5

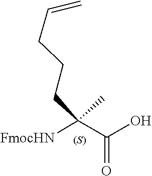

R5

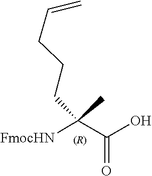

B5

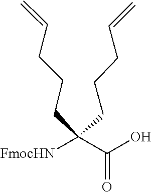

S6

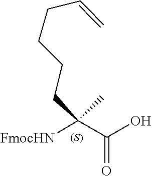

R6

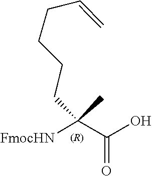

S7

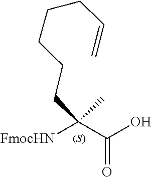

R7

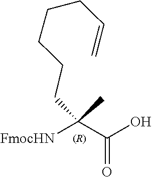

S8

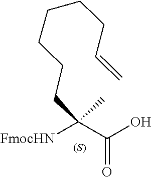

R8

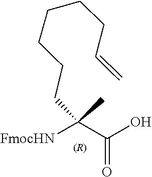

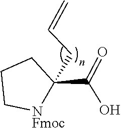

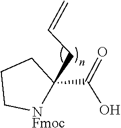

PL3

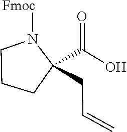

PyrS

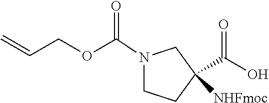

PyrS1

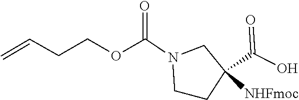

PyrS2

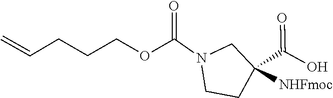

PyrS3

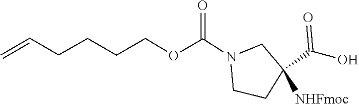

RdN

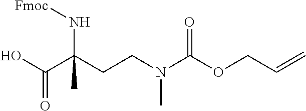

RcN

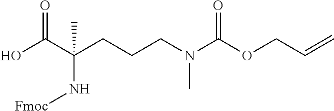

RgN

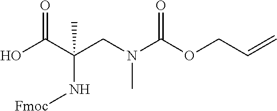

S₁₀

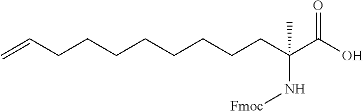

SdN

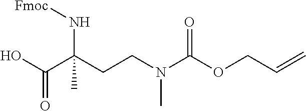

ScN

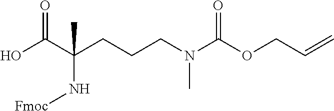

SgN

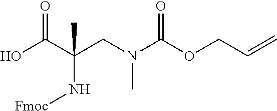

In some embodiments, an amino acid is an alpha-amino acid. In some embodiments, an amino acid is an L-amino acid. In some embodiments, an amino acid is a D-amino acid. In some embodiments, the alpha-carbon of an amino acid is achiral. In some embodiments, an amino acid is a beta-amino acid. In some embodiments, an amino acid is a gamma-amino acid.

In some embodiments, a provided amino acid sequence contains two or more amino acid residues whose side chains are linked together to form one or more staples. In some embodiments, a provided amino acid sequence contains two or more amino acid residues, each of which independently has a side chain comprising an olefin. In some embodiments, a provided amino acid sequence contains two or more amino acid residues, each of which independently has a side chain comprising a terminal olefin. In some embodiments, a provided amino acid sequence contains two and no more than two amino acid residues, each of which independently has a side chain comprising an olefin. In some embodiments, a provided amino acid sequence contains two and no more than two amino acid residues, each of which independently has a side chain comprising a terminal olefin. In some embodiments, a provided amino acid sequence comprises at least one residue of an amino acid that comprises an olefin and a nitrogen atom other than the nitrogen atom of its amino group. In some embodiments, a provided amino acid sequence comprises at least one residue of an amino acid that comprises a terminal olefin and a nitrogen atom other than the nitrogen atom of its amino group. In some embodiments, a provided amino acid sequence comprises at least one residue of an amino acid that has a side chain than comprises a terminal olefin and a nitrogen atom. In some embodiments, a provided amino acid sequence comprises at least one residue of an amino acid of formula A-I, wherein R^{a2} comprising an olefin and a —N(R′)— moiety, wherein R′ is as described in the present disclosure (including, in some embodiments, optionally taken together with R^{a3} and their intervening atoms to form an optionally substituted ring as described in the present disclosure). In some embodiments, R^{a2} comprising a terminal olefin and a —N(R′)— moiety wherein R′ is as described in the present disclosure. In some embodiments, a provided amino acid sequence comprises at least one residue of an amino acid selected from Table A-I. In some embodiments, a provided amino acid sequence comprises at least one residue of an amino acid selected from Table A-II. In some embodiments, a provided amino acid sequence comprises at least one residue of an amino acid selected from Table A-III. In some embodiments, two olefins from two side chains are linked together through olefin metathesis to form a staple. In some embodiments, a staple is preferably formed by side chains of amino acid residues that are not at the corresponding positions of a target of interest. In some embodiments, a formed staple does not disrupt interaction between the peptide and a target of interest.

In some embodiments, a provided staple is a hydrocarbon staple. In some embodiments, a hydrocarbon staple comprises no chain heteroatoms wherein a chain of a staple is the shortest covalent connection within the staple from one end of the staple to the other end of the staple.

In some embodiments, an olefin in a staple is a Z-olefin. In some embodiments, an olefin in a staple in an E-olefin. In some embodiments, a provided composition comprises stapled peptides comprising a staple that contains a Z-olefin and stapled peptides comprising a staple that contains an E-olefin. In some embodiments, a provided composition comprises stapled peptides comprising a staple that contains a Z-olefin. In some embodiments, a provided composition comprises stapled peptides comprising a staple that contains an E-olefin. In some embodiments, otherwise identical stapled peptides that differ only in the E Z configuration of staple olefin demonstrate different properties and/or activities as demonstrated herein. In some embodiments, stapled peptides with E-olefin in a staple may provide certain desirable properties and/or activities given the context. In some embodiments, stapled peptides with Z-olefin in a staple may provide certain desirable properties and/or activities given the context.

In some embodiments, the present disclosure provides compositions comprising stapled peptides. In some embodiments, a composition comprises one and only one stereoisomer of a stapled peptide (e.g., E or Z isomer, and/or a single diastereomer/enantiomer with respect to a chiral center, etc.). In some embodiments, a composition comprises two or more stereoisomers (e.g., both E and Z isomers of one or more double bonds, and/or one or more diastereomers/enantiomers with respect to a chiral center, etc.). In some embodiments, a composition corresponds to a single peak in a chromatographic separation, e.g., HPLC. In some embodiments, a peak comprises one and only one stereoisomers. In some embodiments, a peak comprises two or more stereoisomers.

In some embodiments, two staples may be bonded to the same atom of the peptide backbone, forming a stitched peptide.

In some embodiments, a staple is pro-lock wherein one end of the staple is bonded to the alpha-carbon of a proline residue.

In some embodiments, a staple is a staple illustrated below in Tables S-1, S-2, S-3, S-4 and S-5 (with exemplary peptide backbone illustrated for clarity (can be applied to other peptide backbone), each X independently being an amino acid residue). In some embodiments, a staple is a staple in Table S-6 (with amino acid residues bonded to staples illustrated). In some embodiments, the olefin is Z. In some embodiments, the olefin is E. In some embodiments, an (i, i+3) staple is selected from Table S-1. In some embodiments, an (i, i+3) staple is selected from Table S-2. Those skilled in the art reading the present disclosure will appreciate that when staples in Table S-1 and Table S-2 are utilized for (i, i+3), “X₃” in those tables would be “X₂” (i.e., two amino acid residues instead of three amino acid residues). In some embodiments, an (i, i+4) staple is selected from Table S-1. In some embodiments, an (i, i+4) staple is selected from Table S-2. In some embodiments, an (i, i+7) staple is selected from Table S-3. In some embodiments, an (i, i+7) staple is selected from Table S-4.

TABLE S-1
Exemplary staples.

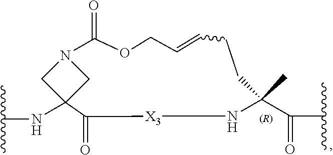

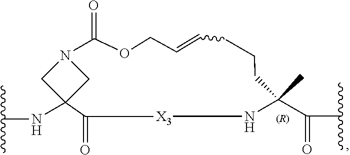

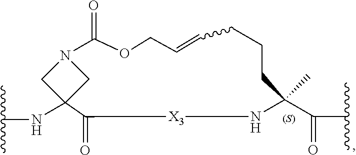

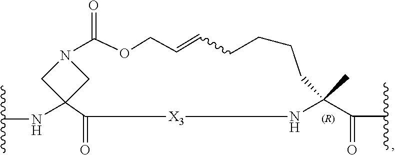

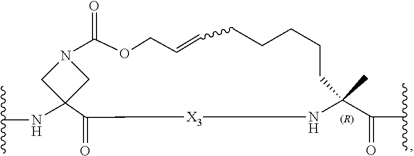

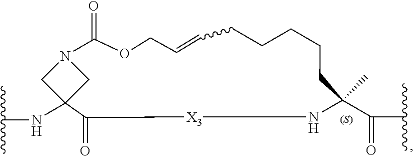

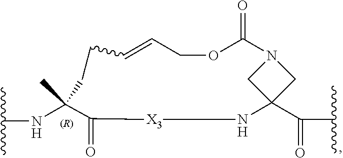

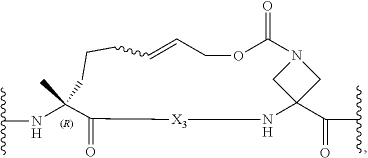

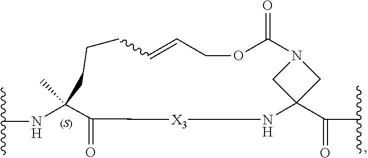

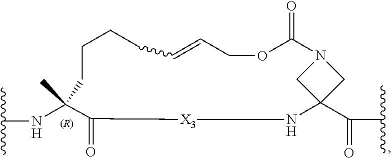

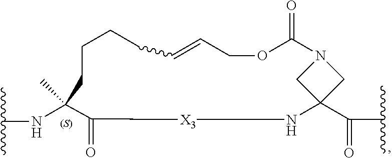

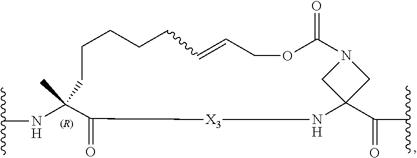

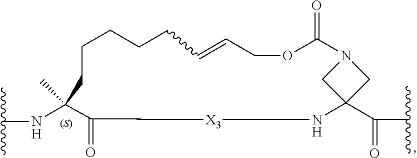

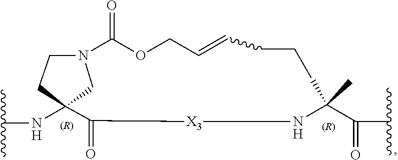

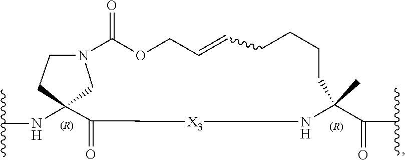

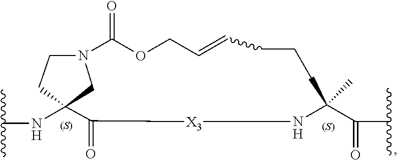

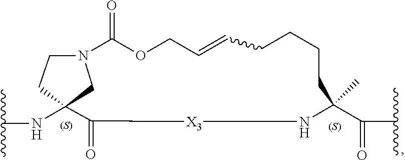

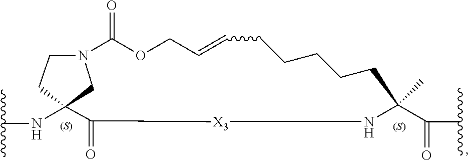

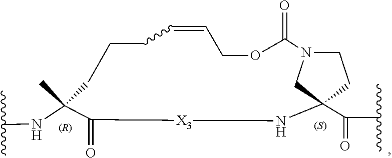

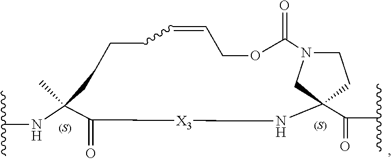

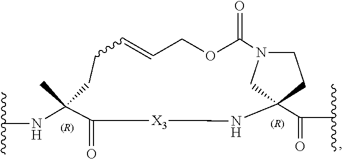

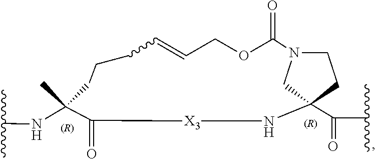

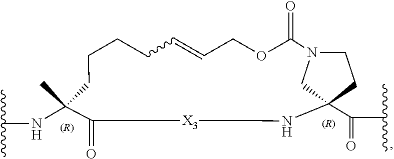

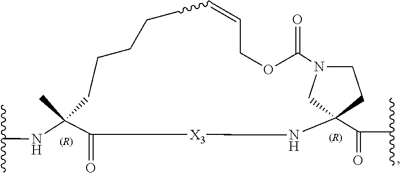

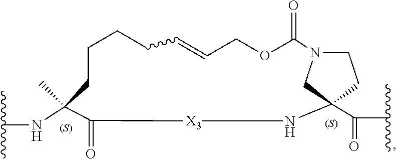

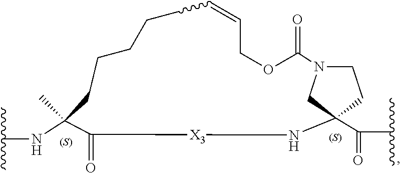

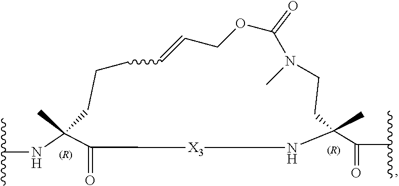

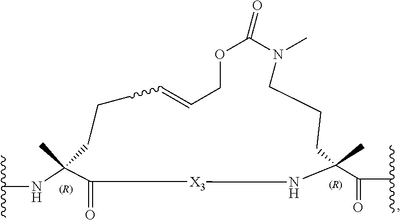

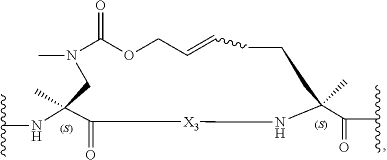

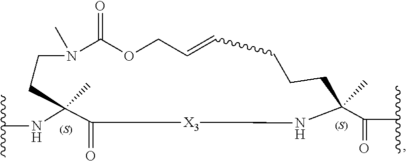

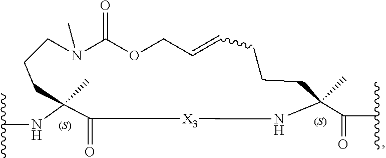

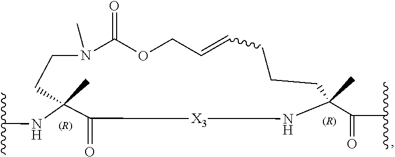

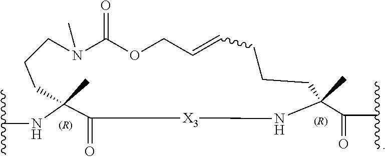

TABLE S-2
Exemplary staples.

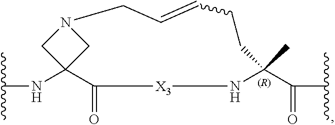

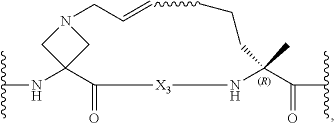

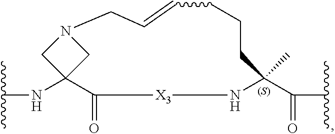

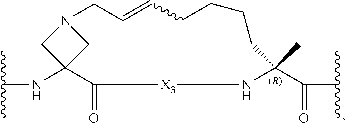

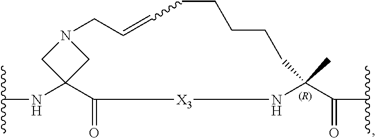

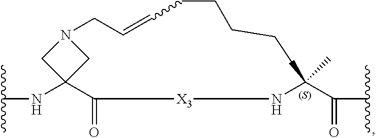

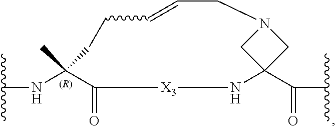

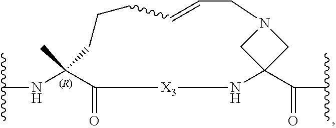

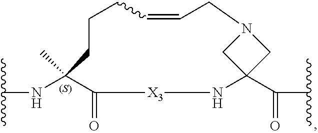

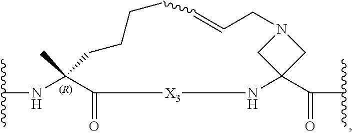

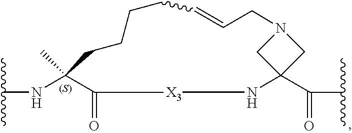

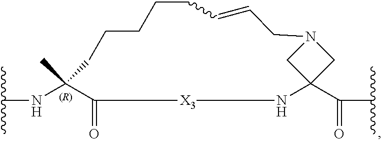

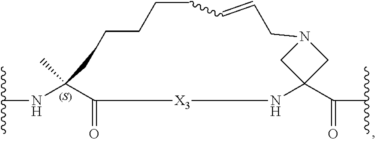

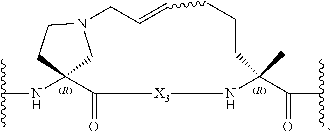

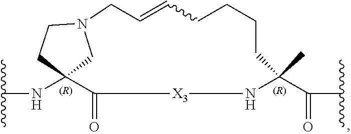

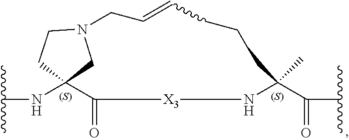

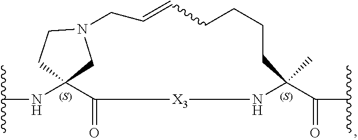

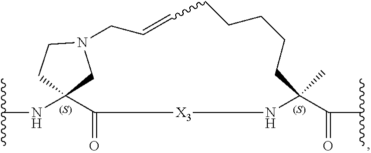

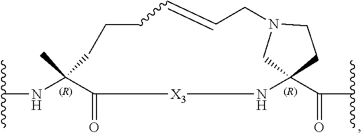

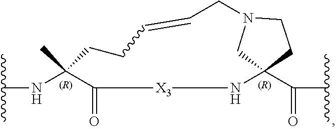

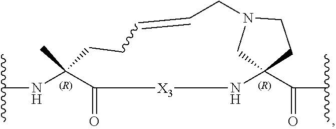

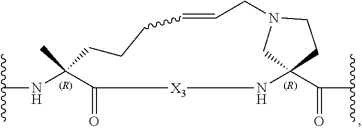

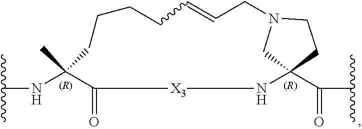

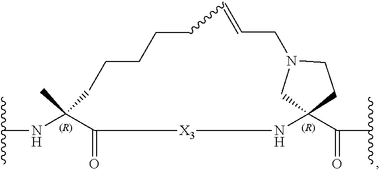

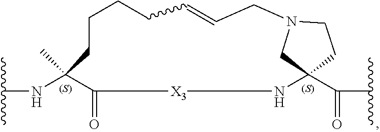

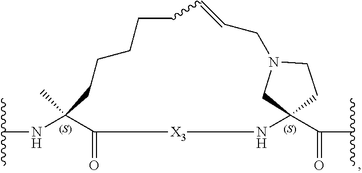

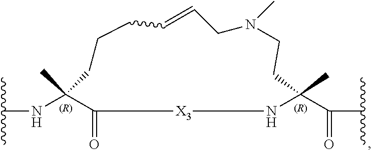

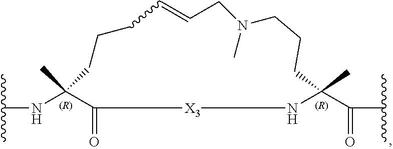

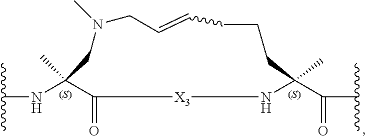

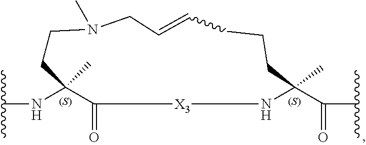

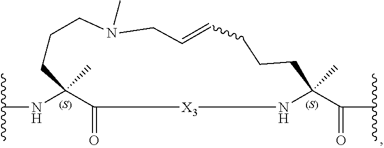

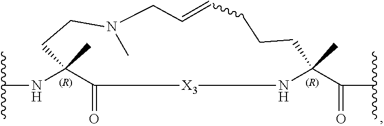

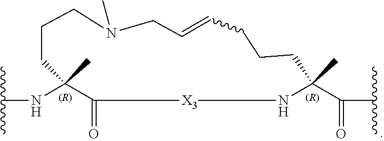

TABLE S-3
Exemplary staples.

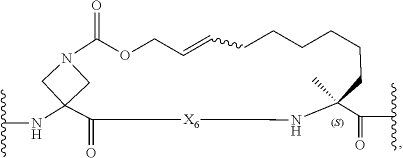

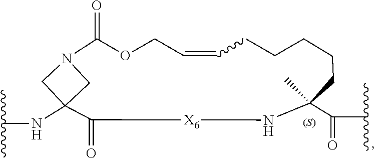

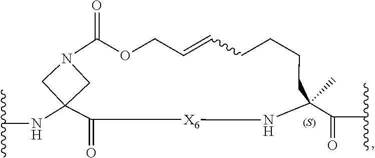

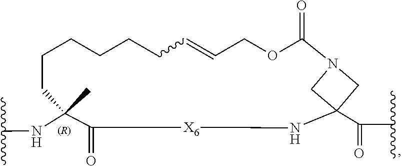

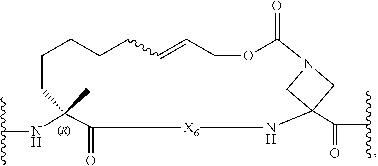

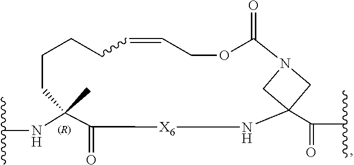

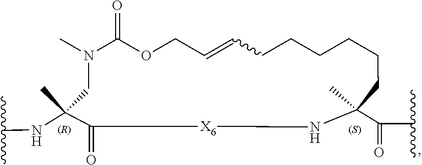

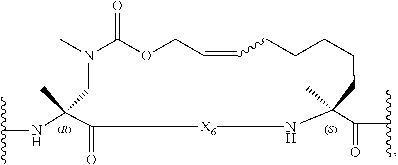

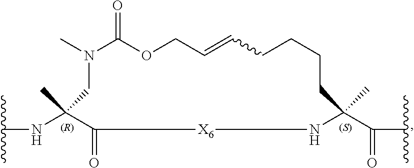

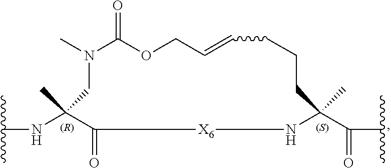

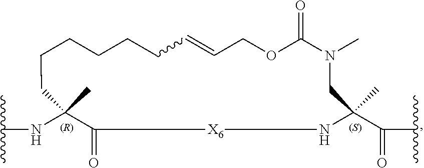

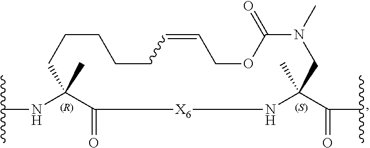

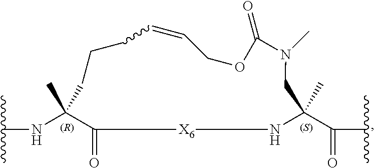

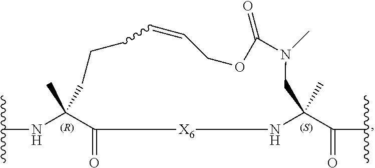

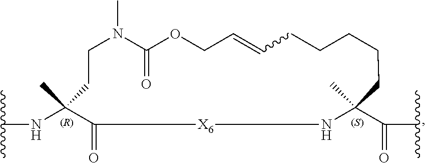

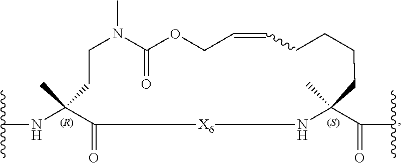

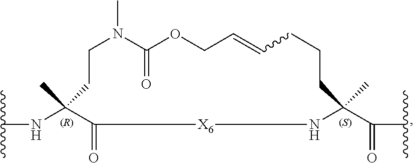

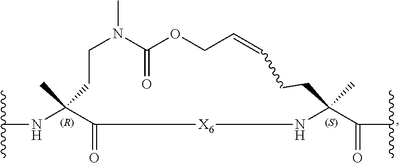

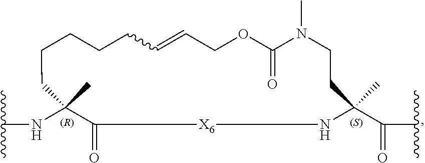

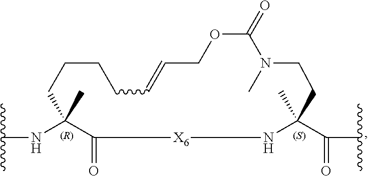

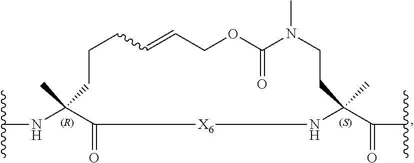

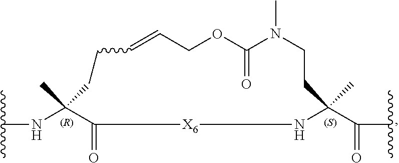

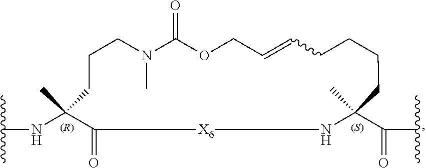

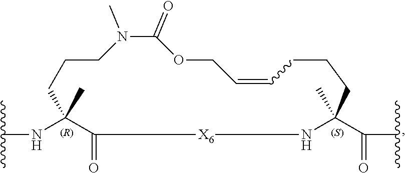

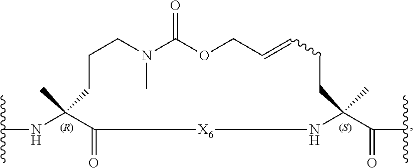

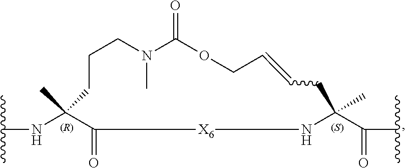

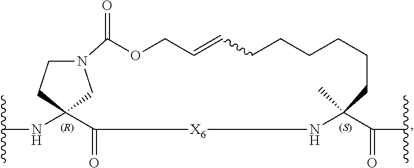

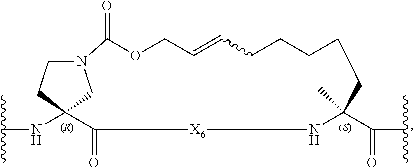

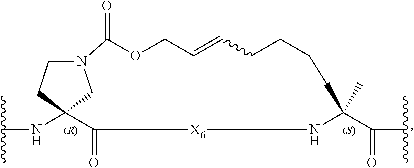

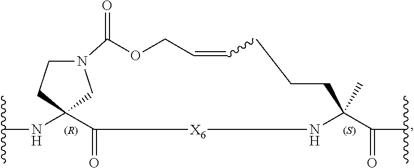

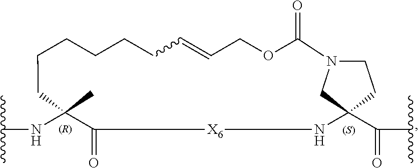

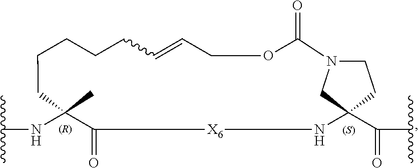

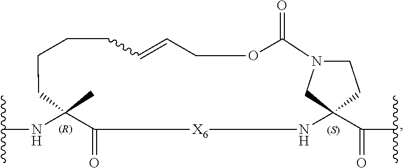

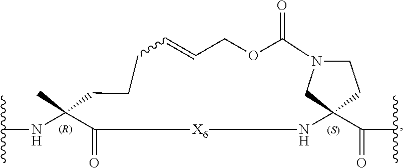

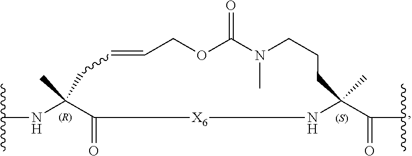

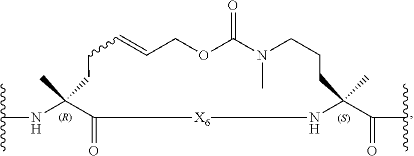

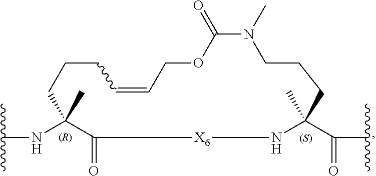

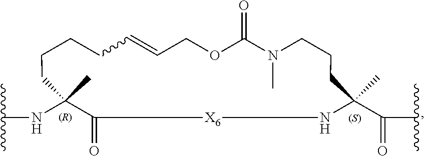

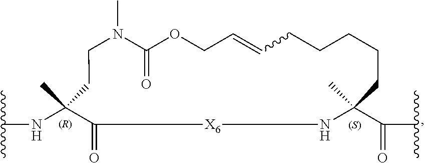

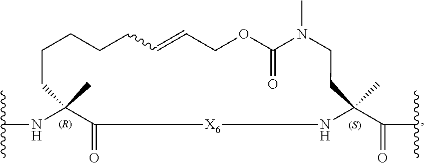

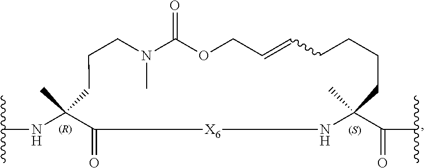

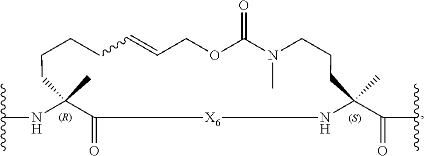

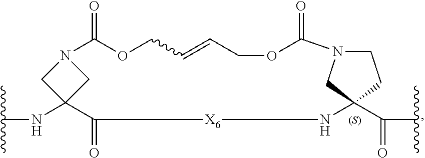

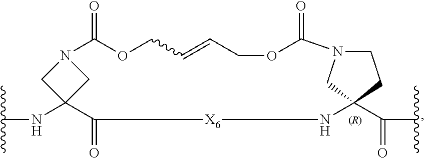

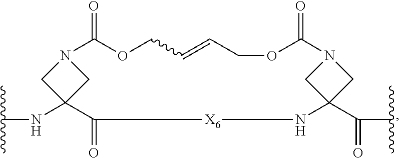

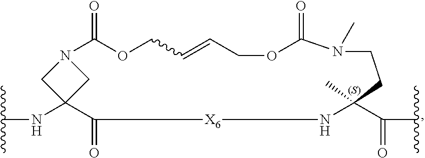

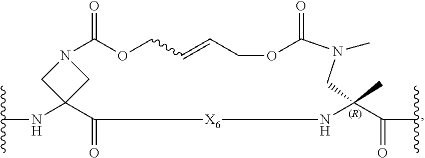

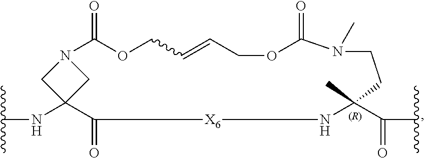

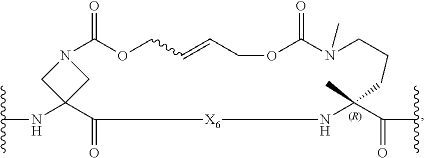

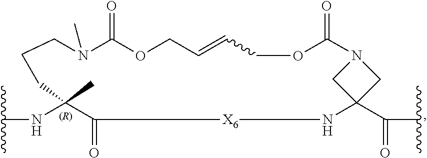

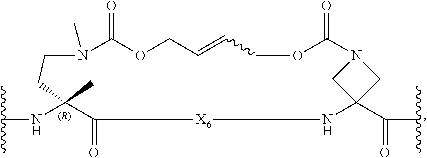

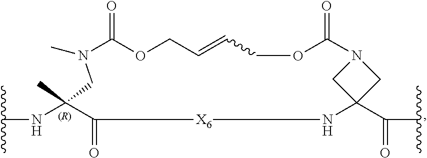

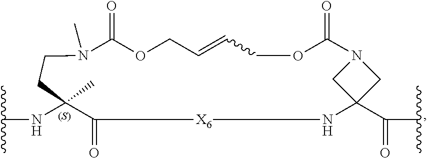

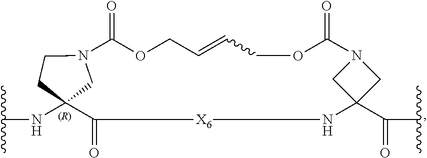

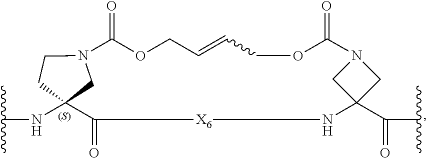

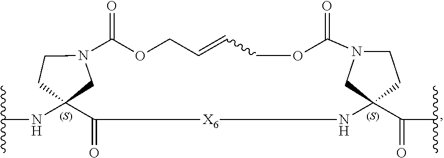

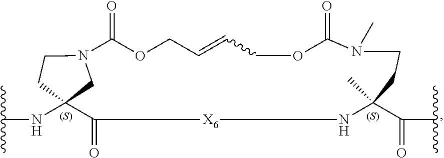

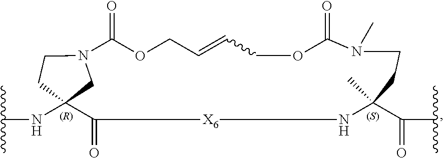

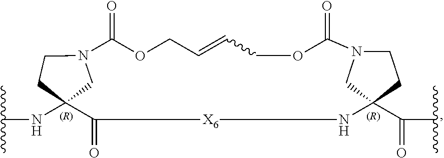

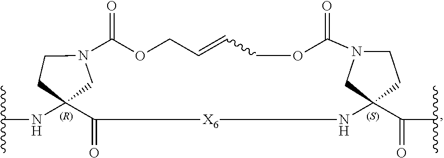

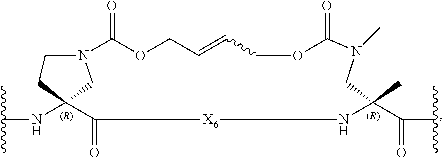

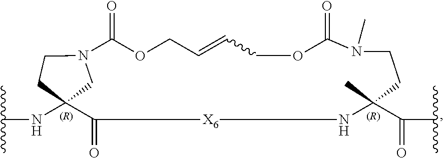

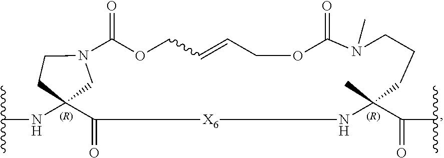

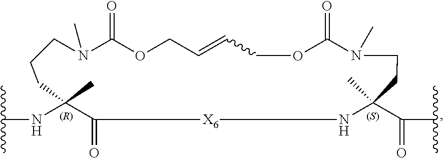

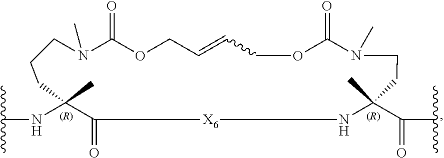

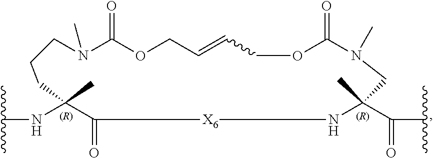

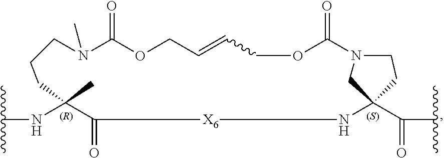

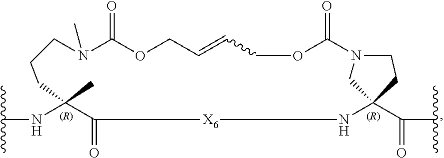

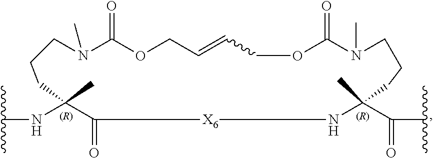

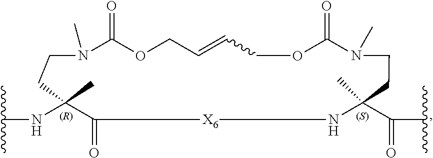

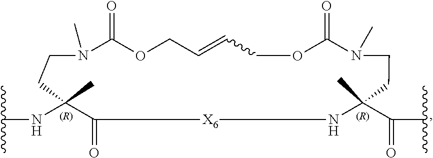

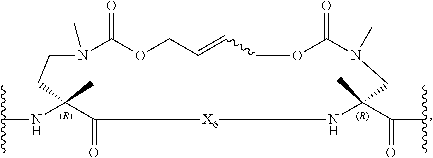

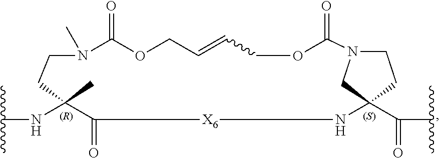

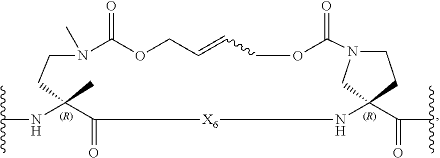

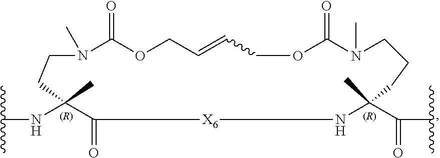

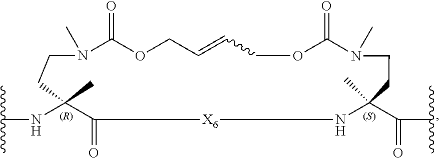

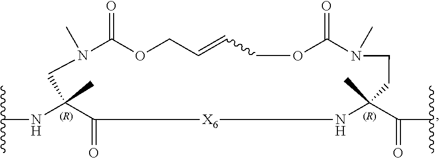

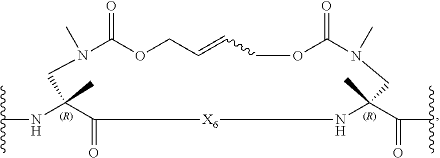

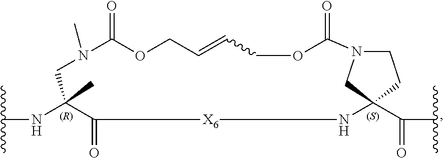

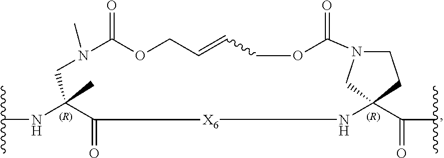

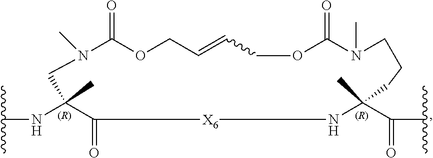

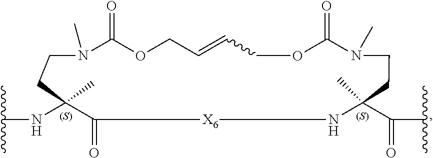

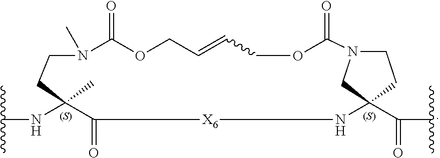

TABLE S-4
Exemplary staples.

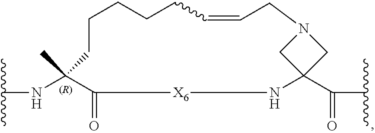

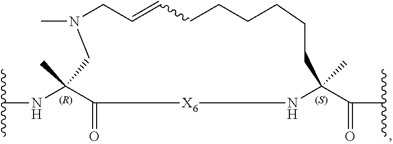

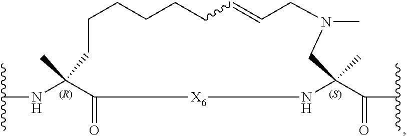

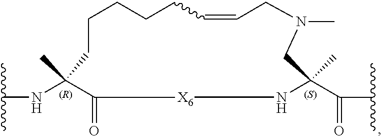

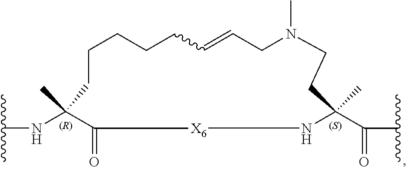

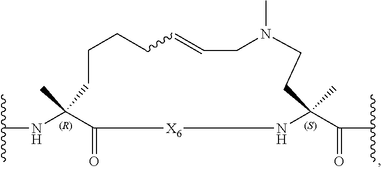

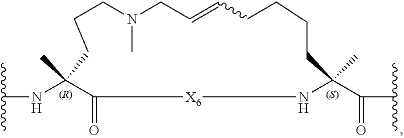

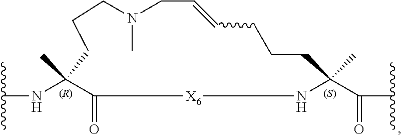

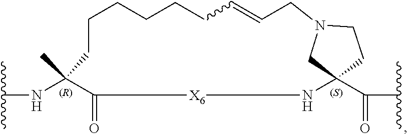

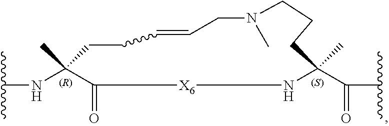

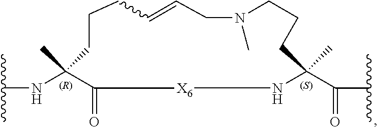

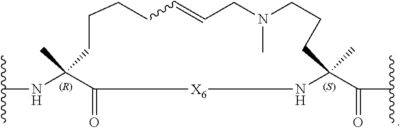

Certain useful staples are described in, e.g., WO 2019/051327, WO 2022/020652, etc. and are incorporated herein by reference.

In some embodiments, a staple may be one of the following, connecting the amino acids at the indicated position:

TABLE S-5
Certain amino acids and staples.

| Amino Acid | Amino Acid 2 | |
|---|---|---|
| 1 | (i + 7 | staple |
| Monomer A | S₈ | 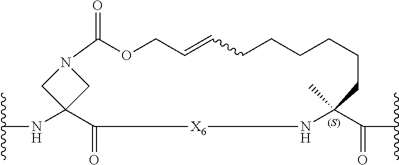 |
| Monomer A | S₇ | 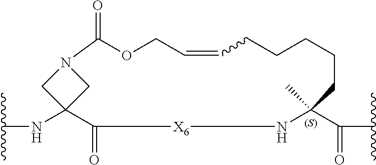 |
| Monomer A | S₅ | 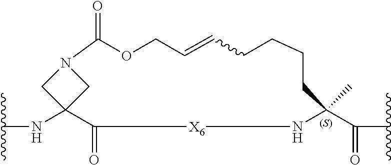 |
| R₈ | Monomer A | 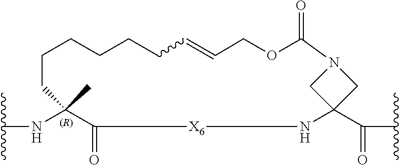 |
| R₇ | Monomer A | 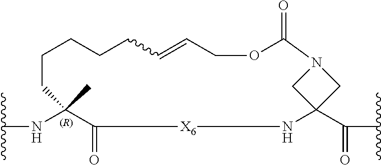 |
| R₆ | Monomer A | 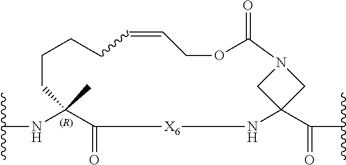 |
| Monomer E | S₈ | 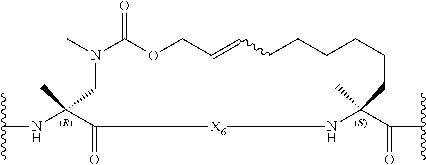 |
| Monomer E | S₇ | 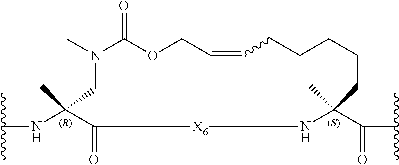 |
| Monomer E | S₆ | 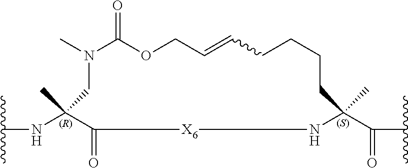 |
| Monomer E | S₅ | 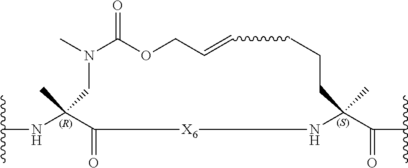 |
| R₈ | Monomer D | 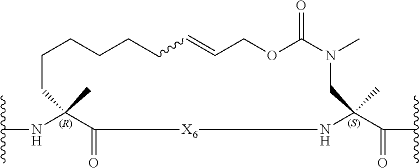 |
| R₇ | Monomer D | 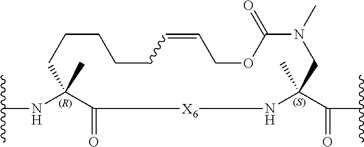 |
| R₆ | Monomer D | 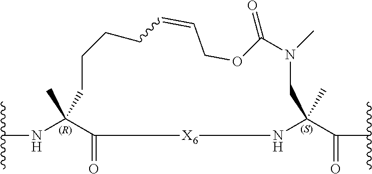 |
| R₅ | Monomer D | 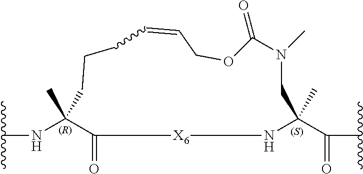 |
| Monomer G | S₇ | 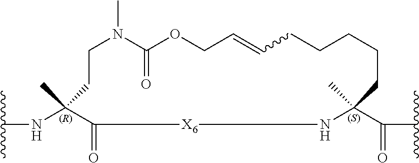 |
| Monomer G | S₆ | 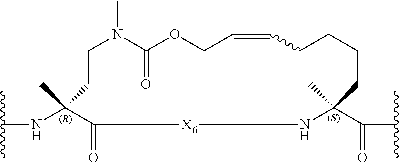 |
| Monomer G | S₅ | 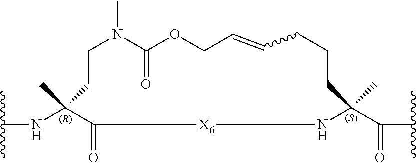 |
| Monomer G | S₄ | 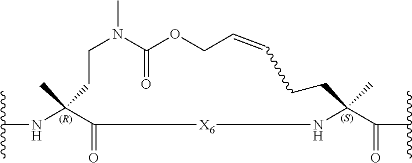 |
| R₇ | Monomer F | 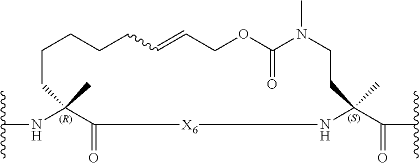 |
| R₆ | Monomer F | 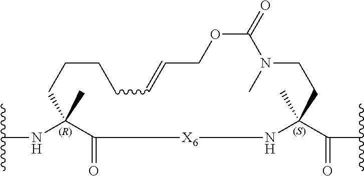 |
| R₅ | Monomer F | 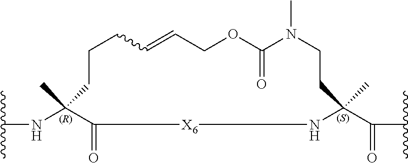 |
| R₅ | Monomer F | 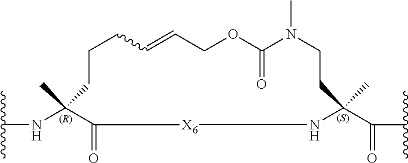 |
| R₄ | Monomer F | 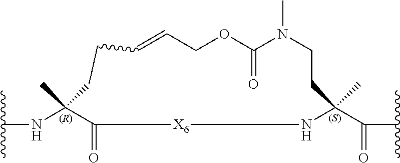 |
| Monomer I | S₆ | 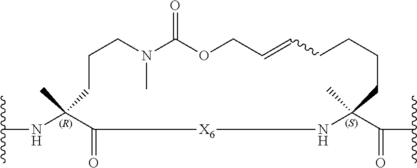 |
| Monomer I | S₅ | 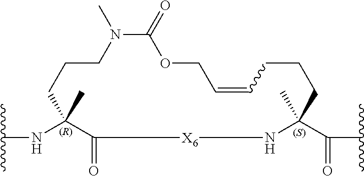 |
| Monomer I | S₄ | 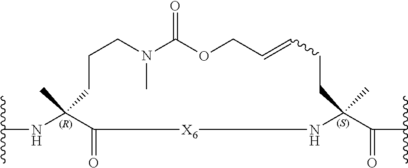 |
| Monomer I | S₃ | 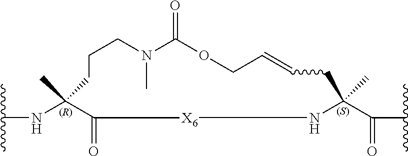 |
| Monomer C | S₈ | 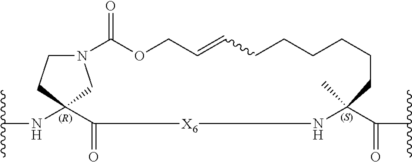 |
| Monomer C | S₇ | 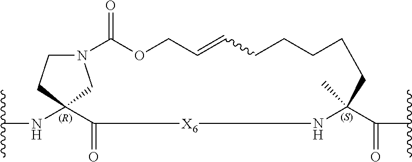 |
| Monomer C | S₆ | 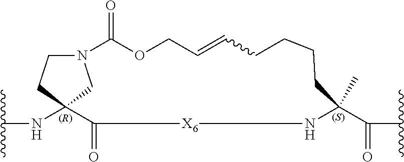 |
| Monomer C | S₅ | 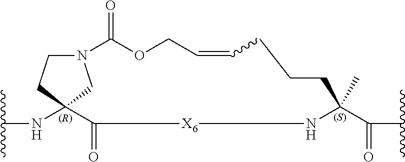 |
| R₈ | Monomer B | 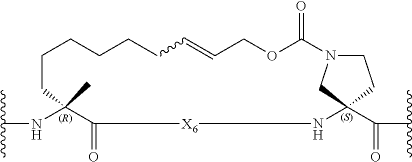 |
| R₇ | Monomer B | 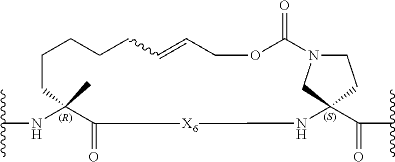 |
| R₆ | Monomer B | 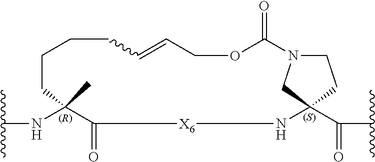 |
| R₅ | Monomer B | 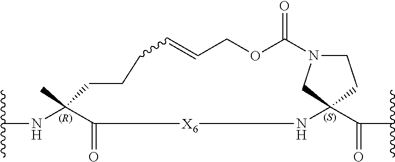 |
| R₃ | Monomer H | 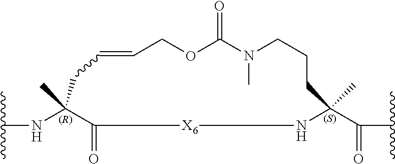 |
| R₄ | Monomer H | 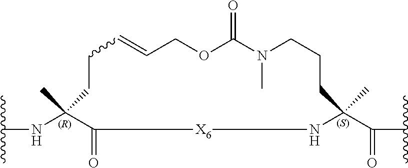 |
| R₅ | Monomer H | 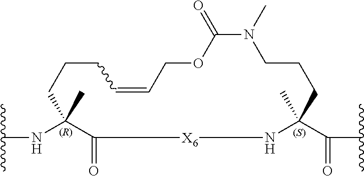 |
| R₆ | Monomer H | 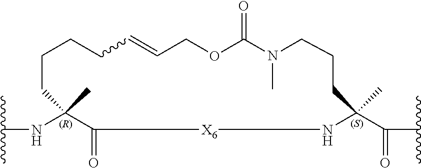 |
| Monomer G | S₇ | 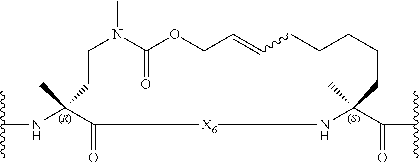 |
| R₇ | Monomer F | 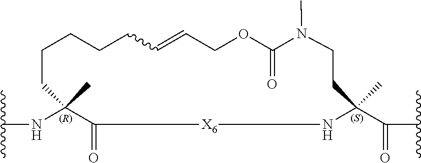 |
| Monomer I | S₆ | 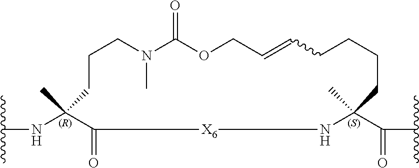 |
| R₆ | Monomer H | 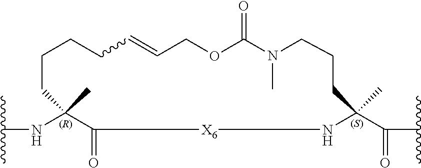 |
| Monomer A | Monomer B | 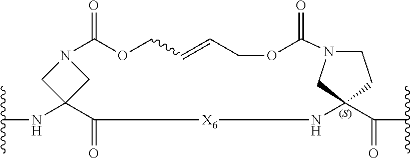 |
| Monomer A | Monomer C | 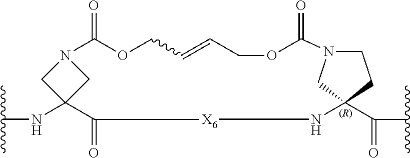 |
| Monomer A | Monomer A | 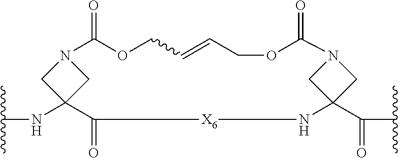 |
| Monomer A | Monomer F | 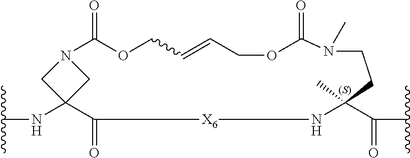 |
| Monomer A | Monomer E | 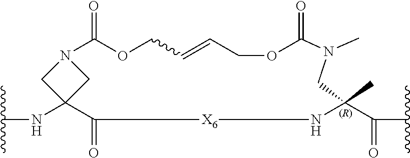 |
| Monomer A | Monomer G | 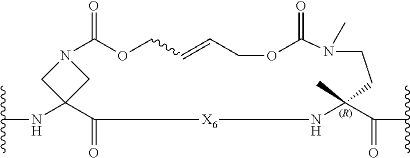 |
| Monomer A | Monomer I | 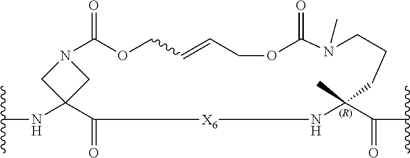 |
| Monomer I | Monomer A | 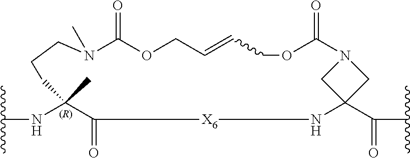 |
| Monomer G | Monomer A | 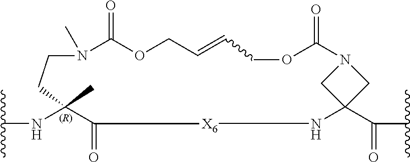 |
| Monomer E | Monomer A | 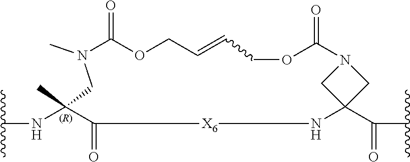 |
| Monomer F | Monomer A | 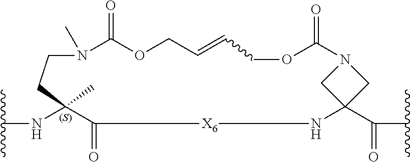 |
| Monomer C | Monomer A | 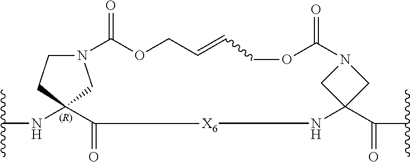 |
| Monomer B | Monomer A | 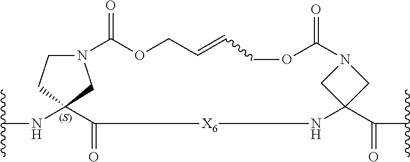 |
| Monomer B | Monomer B | 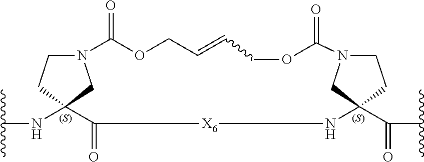 |
| Monomer B | Monomer F | 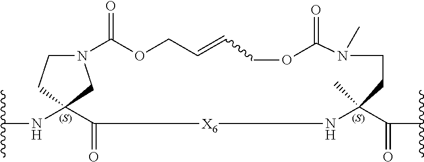 |
| Monomer C | Monomer F | 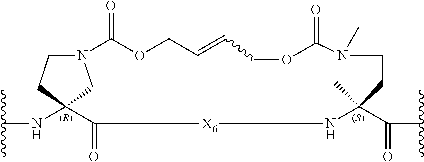 |
| Monomer C | Monomer C | 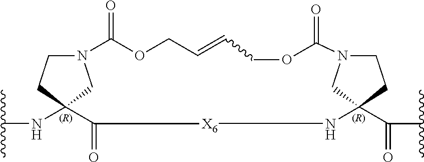 |
| Monomer C | Monomer B | 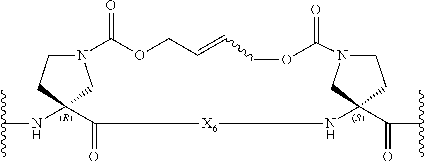 |
| Monomer C | Monomer E | 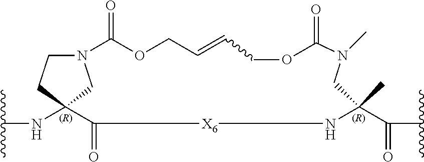 |
| Monomer C | Monomer G | 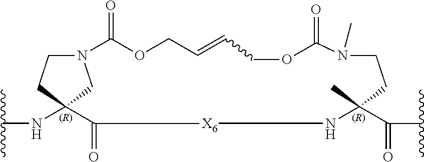 |
| Monomer C | Monomer I | 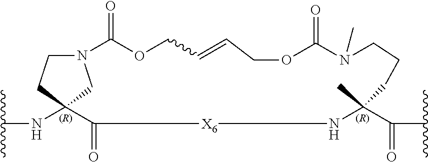 |
| Monomer I | Monomer F | 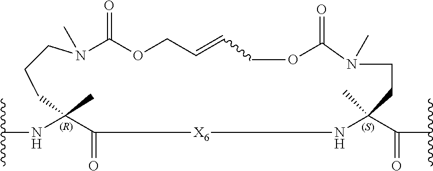 |
| Monomer I | Monomer G | 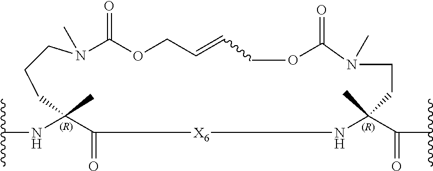 |
| Monomer I | Monomer E | 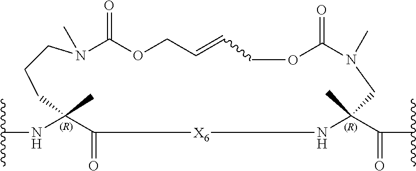 |
| Monomer I | Monomer B | 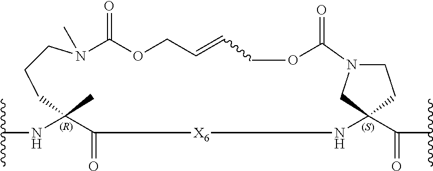 |
| Monomer I | Monomer C | 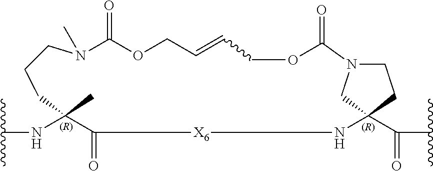 |
| Monomer I | Monomer I | 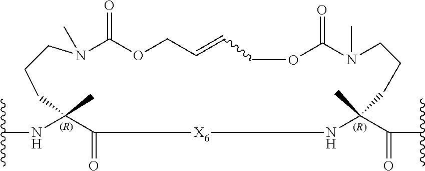 |
| Monomer G | Monomer F | 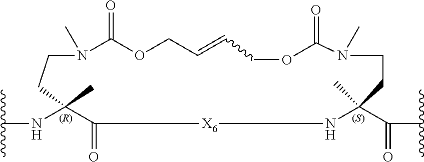 |
| Monomer G | Monomer G | 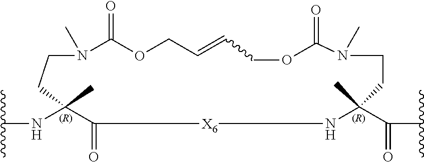 |
| Monomer G | Monomer E | 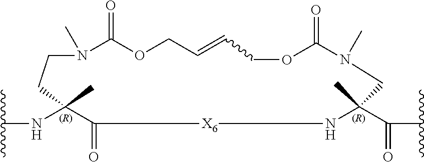 |
| Monomer G | Monomer B | 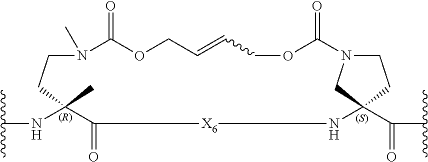 |
| Monomer G | Monomer C | 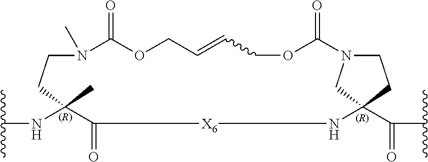 |
| Monomer G | Monomer I | 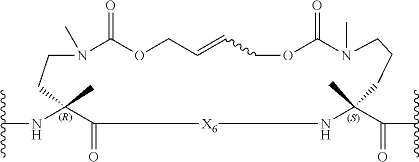 |
| Monomer E | Monomer F | 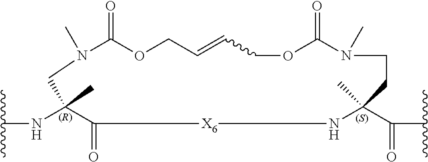 |
| Monomer E | Monomer G | 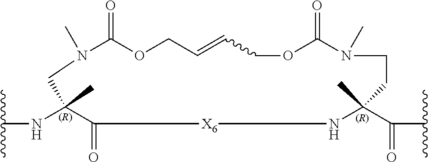 |
| Monomer E | Monomer E | 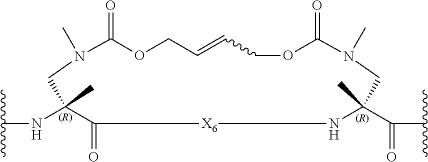 |
| Monomer E | Monomer B | 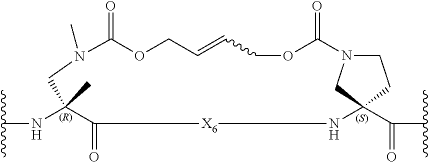 |
| Monomer E | Monomer C | 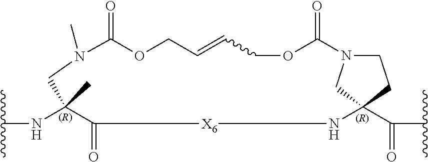 |
| Monomer E | Monomer I | 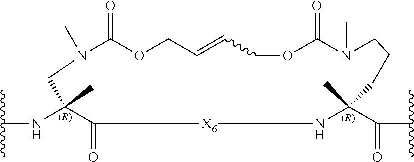 |
| Monomer F | Monomer F | 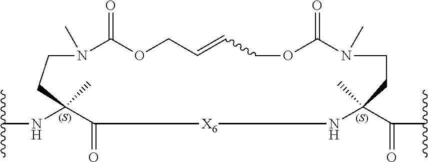 |
| Monomer F | Monomer B | 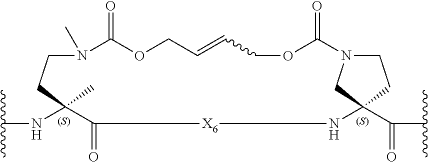 |
| R₇ | Monomer A | 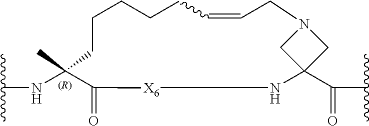 |
| Monomer E | S₈ | 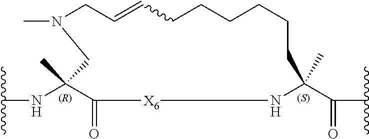 |
| R₈ | Monomer D | 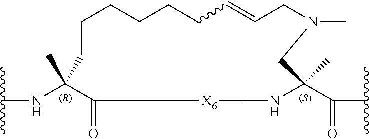 |
| R₇ | Monomer D | 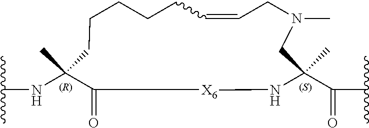 |
| R₇ | Monomer F | 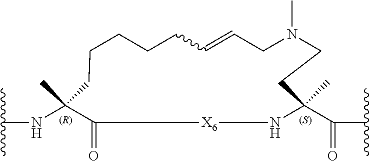 |
| R₆ | Monomer F | 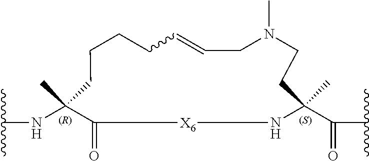 |
| Monomer I | S₆ | 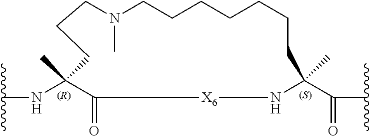 |
| Monomer I | S₅ | 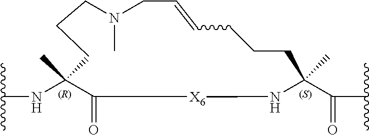 |
| R₈ | Monomer B | 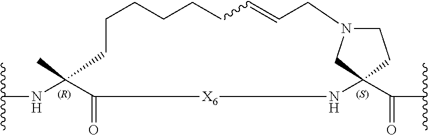 |
| R₄ | Monomer H | 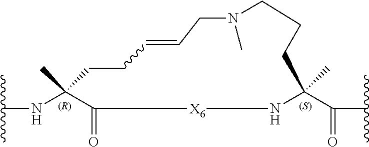 |
| R₅ | Monomer H | 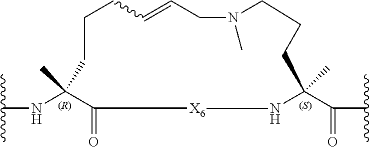 |
| R₆ | Monomer H | 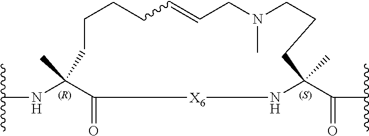 |

In some embodiments, a peptide comprises a staple or stitch (two staples) from Table S-6. In Table 6, the amino acid residues can either be from N to C or C to N. In some embodiments, it is N to C. In some embodiments, it is C to N. In some embodiments, a double bond is E. In some embodiments, a double bond is Z. In some embodiments, a staple is a (i, i+2) staple. In some embodiments, a staple is a (i, i+3) staple. In some embodiments, a staple is a (i, i+4) staple. In some embodiments, a staple is a (i, i+7) staple. In some embodiments, each double is independently E or Z when a structure comprises more than one double bond. In some embodiments, each staple is independently a (i, i+2) or a (i, i+3) or a (i, i+4) staple or a (i, i+7) staple. In some embodiments, each staple is independently a (i, i+2) or a (i, i+4) staple or a (i, i+7) staple. In some embodiments, each staple is independently a (i, i+3) or a (i, i+4) staple or a (i, i+7) staple. In some embodiments, each staple is independently a (i, i+4) staple or a (i, i+7) staple in a structure comprising two staples. In some embodiments, one staple is a (i, i+4) staple and the other is a (i, i+7) staple. In some embodiments, one staple is a (i, i+3) staple, one staple is a (i, i+4) staple and one staple is a (i, i+7) staple. In some embodiments, one staple is a (i, i+2) staple, one staple is a (i, i+4) staple and one staple is a (i, i+7) staple. In some embodiments, a PL3 residue is bonded to a (i, i+3) staple. In some embodiments, a PL3 residue is bonded to a (i, i+4) staple. In some embodiments, staples (e.g., those in Table 6) are formed by metathesis of double bonds in side chains of amino acid residues, e.g., RdN and S7, R8 and PyrS, R5 and SeN, R6 and SeN, ReN and S5, ReN and S6, R7 and PyrS, Az and S7, R8 and SgN, Az and S8, R4 and SeN, R5 and SdN, R7 and Az, R8 and Az, RdN and S4, RgN and S8, RgN and S7, R8 and S5, PL3 and B5 and the same B5 and S8, PL3 and B5 and the same B5 and SeN, PL3 and B5 and the same B5 and SdN, PL3 and B5 and the same B5 and S7, PL3 and B5 and the same B5 and PyrS2, PL3 and B5 and the same B5 and PyrS3, R5 and PyrS2, PL3 and B5 and the same B5 and PyrS1, PL3 and B5 and the same B5 and S10, PL3 and B5 and the same B5 and PyrR2, PL3 and B5 and the same B5 and PyrS, PL3 and B5 and the same B5 and Az, PL3 and B5 and the same B5 and SeNc5, HypEs5 and B5 and the same B5 and PyrS2, HypEs4 and B5 and the same B5 and PyrS2, ProSAm3 and B5 and the same B5 and PyrS2, ProAm5 and B5 and the same B5 and PyrS2, ProAm6 and B5 and the same B5 and PyrS2, BzAm30allyl and B5 and the same B5 and PyrS2, HypBzEs30Allyl and B5 and the same B5 and PyrS2, ProBzAm30Allyl and B5 and the same B5 and PyrS2, PAc30Allyl and B5 and the same B5 and PyrS2, ProPAc30Allyl and B5 and the same B5 and PyrS2, HypPAc30Allyl and B5 and the same B5 and PyrS2, Bn30Allyl and B5 and the same B5 and PyrS2, R3 and B5 and the same B5 and PyrS2, R5 and B5 and the same B5 and PyrS2, [BzAm2Allyl]MePro and B5 and the same B5 and PyrS2, PL3 and B5 and the same B5 and SPip1, PL3 and B5 and the same B5 and SPip2, PL3 and B5 and the same B5 and SPip3, PL3 and B5 and the same B5 and Az2, PL3 and B5 and the same B5 and Az3, PL3 and S5, R5 and S5, PL3 and B4 and the same B4 and PyrS1, PL3 and B4 and the same B4 and PyrS2, PL3 and B4 and the same B4 and PyrS3, PL3 and S6, PL3 and S4, PL3 and S3, R6 and PyrS2, R4 and PyrS2, R3 and PyrS2, PL3 and B3 and the same B3 and PyrS2, PL3 and B3 and the same B3 and PyrS3, PL3 and B3 and the same B3 and PyrS4, PL3 and B6 and the same B6 and PyrS, PL3 and B6 and the same B6 and PyrS1, PL3 and B6 and the same B6 and PyrS2.

TABLE S-6
Certain staples (including amino acid residues bonded to staples).

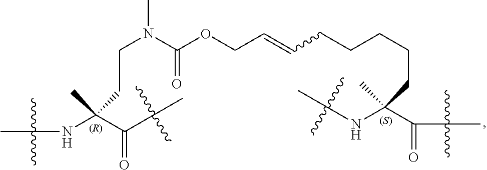

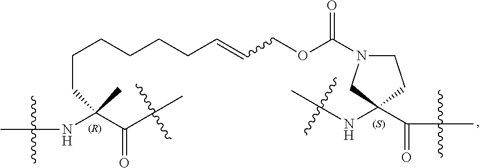

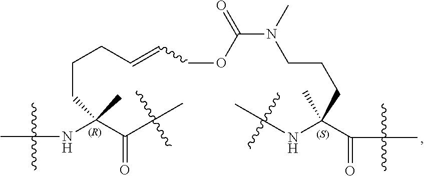

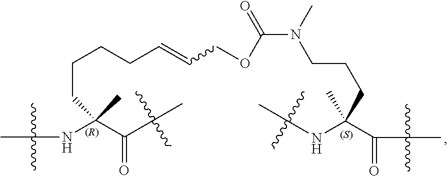

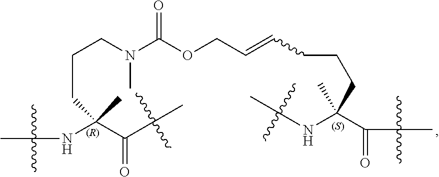

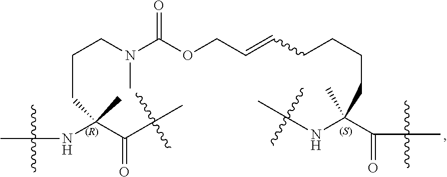

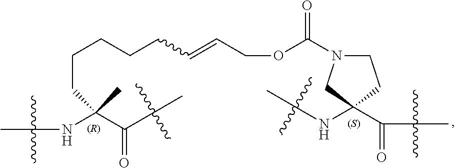

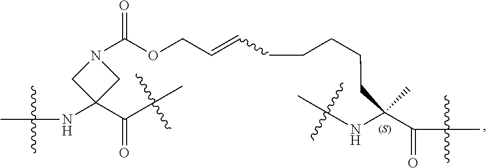

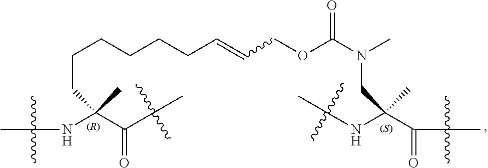

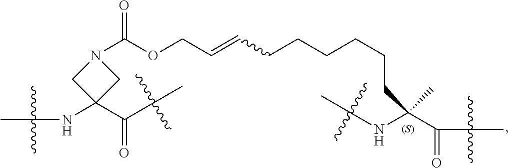

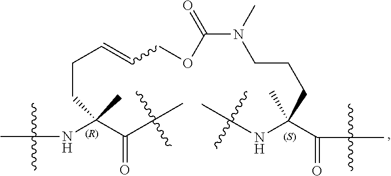

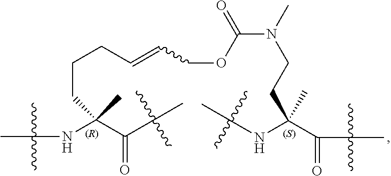

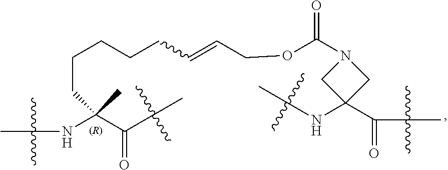

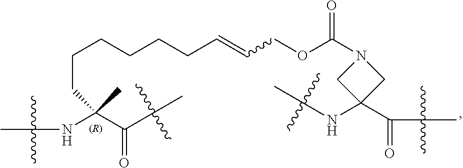

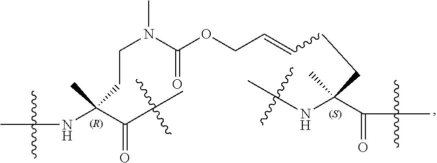

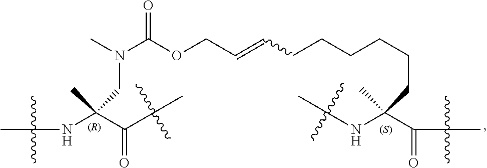

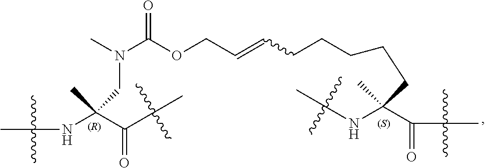

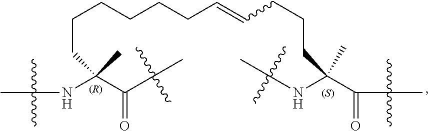

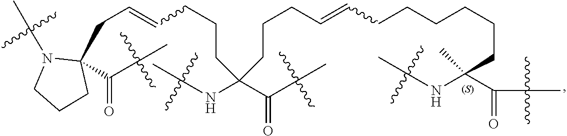

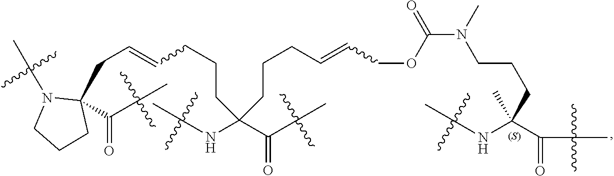

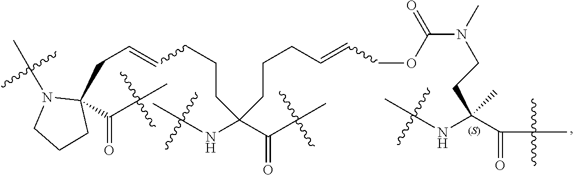

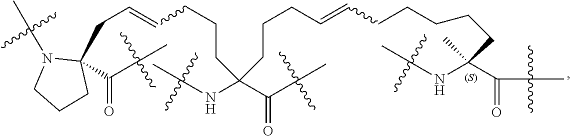

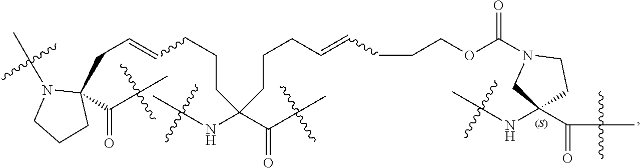

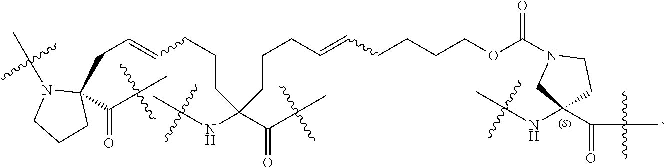

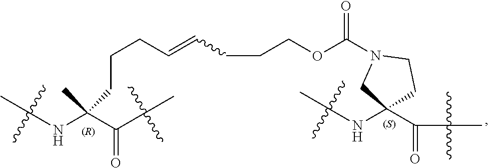

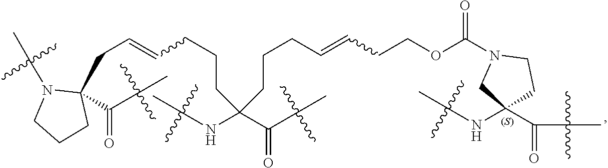

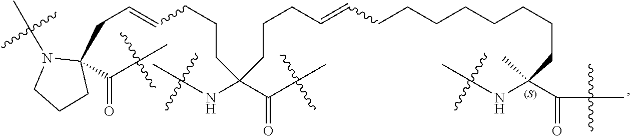

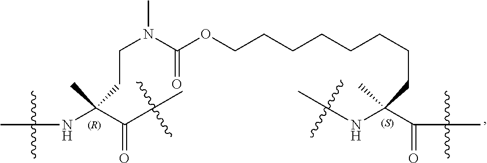

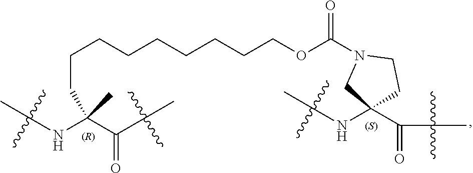

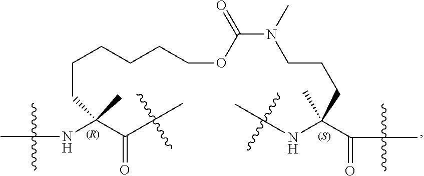

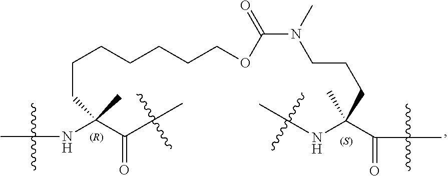

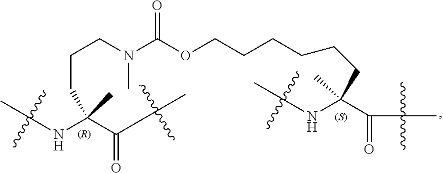

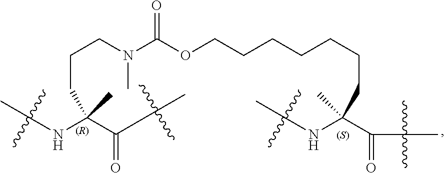

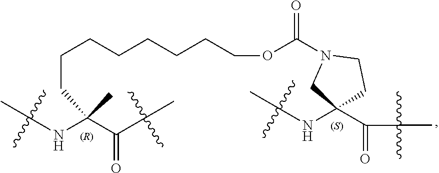

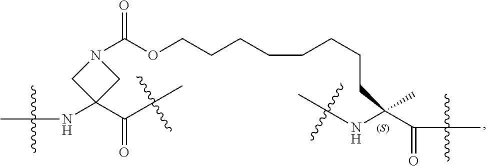

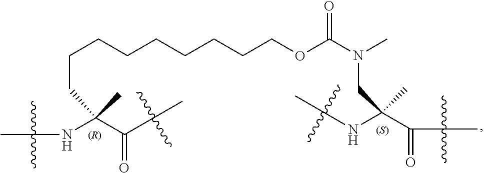

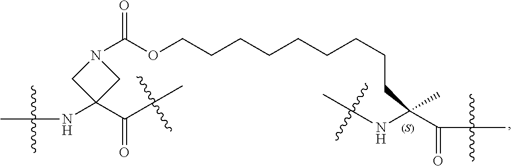

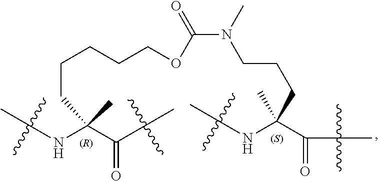

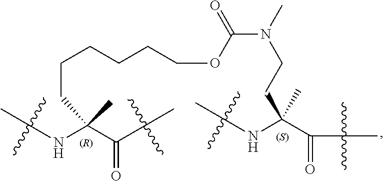

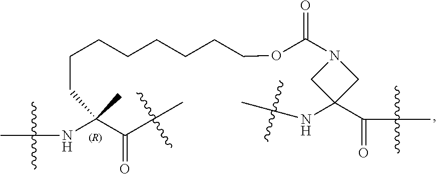

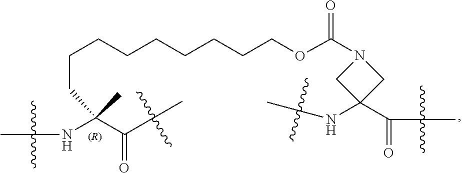

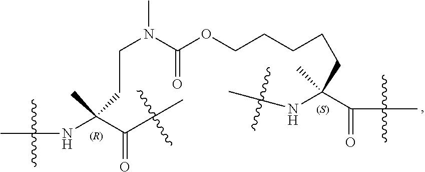

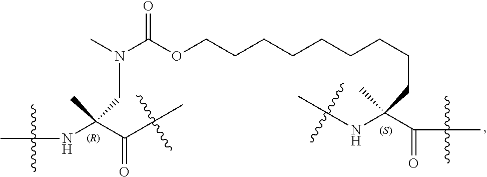

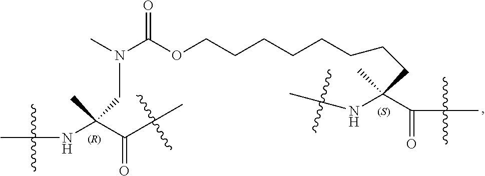

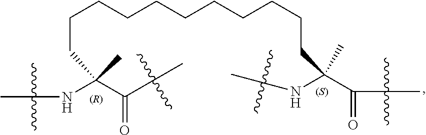

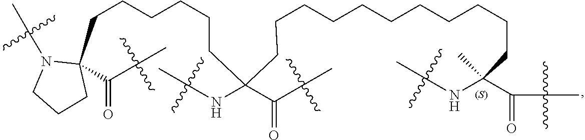

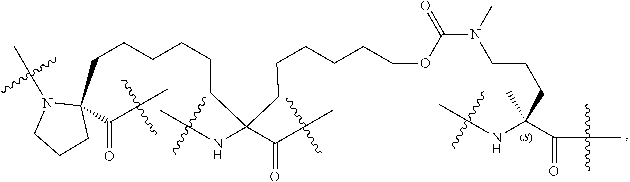

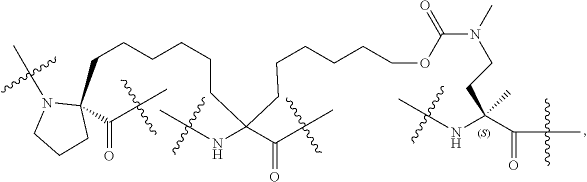

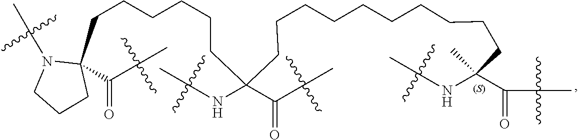

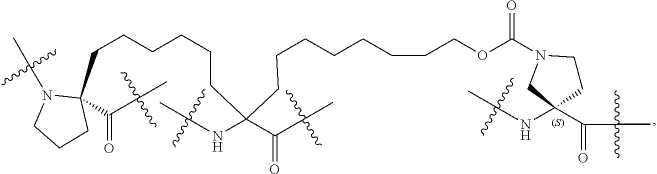

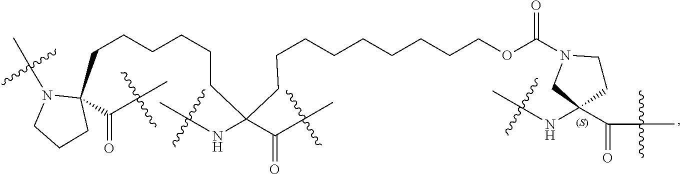

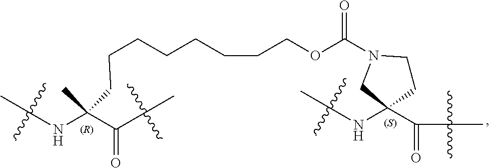

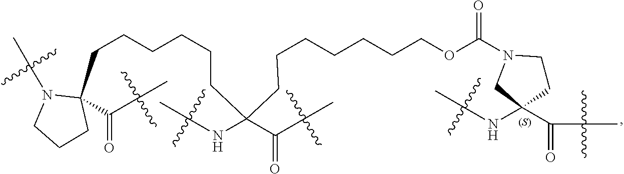

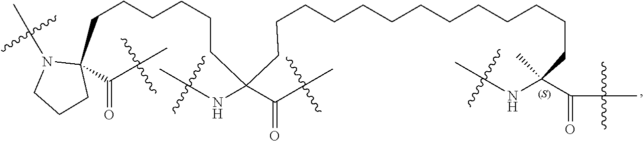

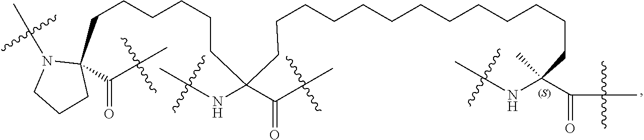

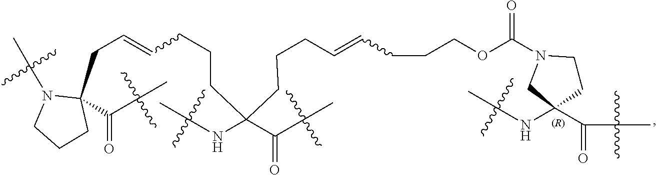

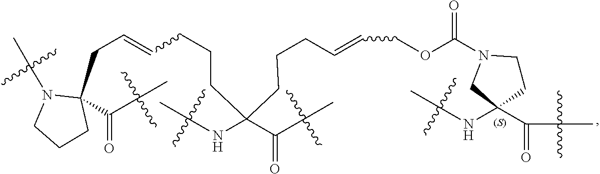

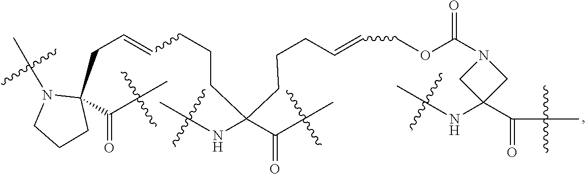 [SMILES: *NC(CCCC=CCOC(=O)N1CC(N*)(C(*)=O)C1)(CCCC=CC[C@@]1(C(*)=O)CCCN1*)C(*)=O]

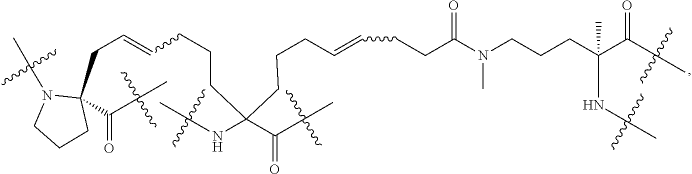 [SMILES: *NC(CCCC=CCCC(=O)N(C)CCC[C@](C)(N*)C(*)=O)(CCCC=CC[C@@]1(C(*)=O)CCCN1*)C(*)=O]

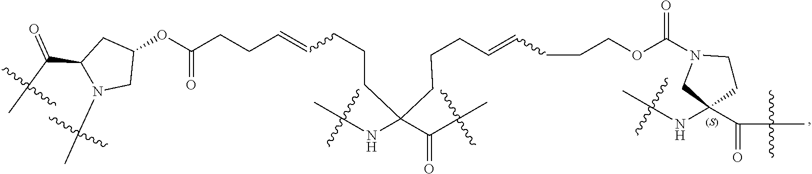

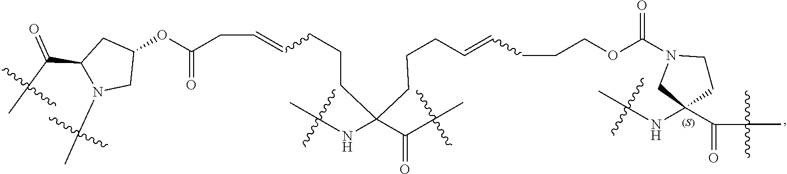

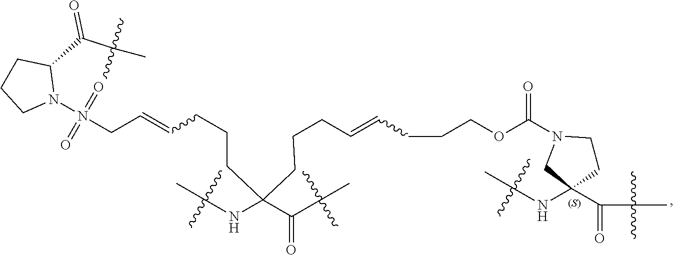

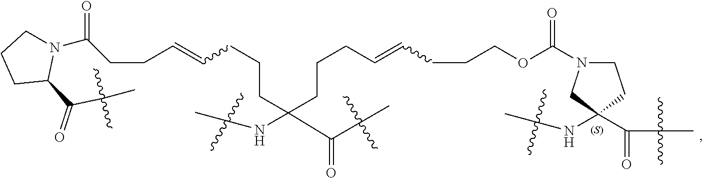

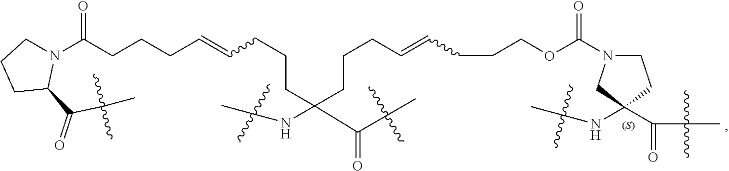

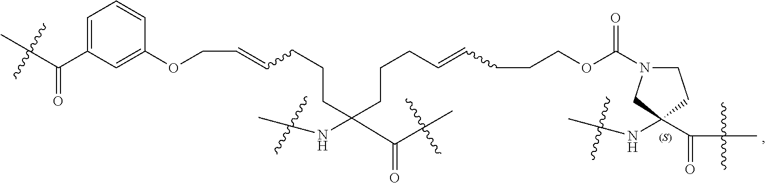

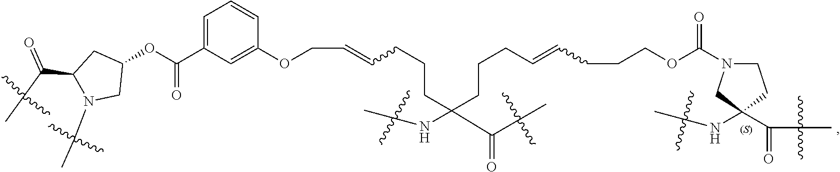

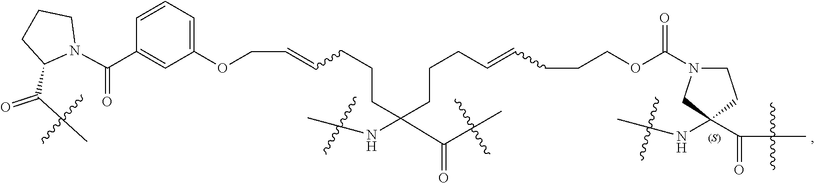

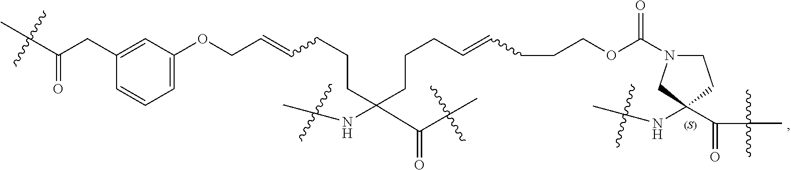

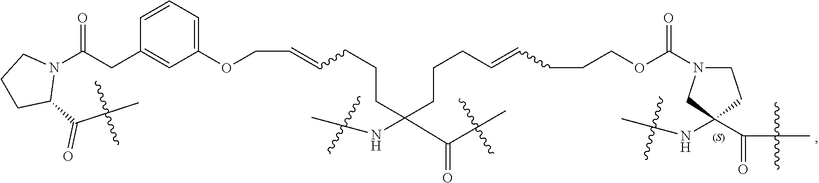

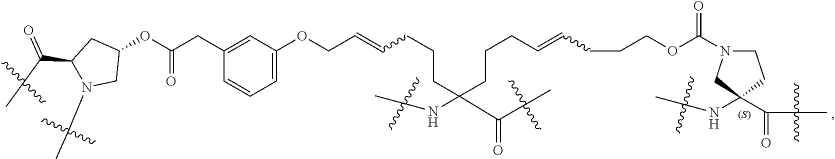

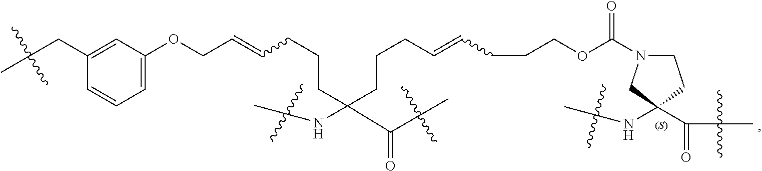

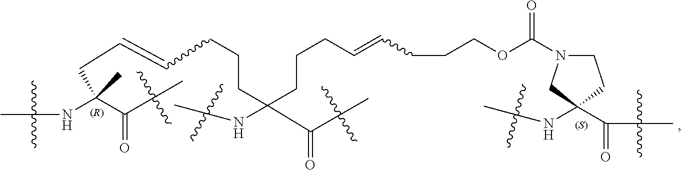

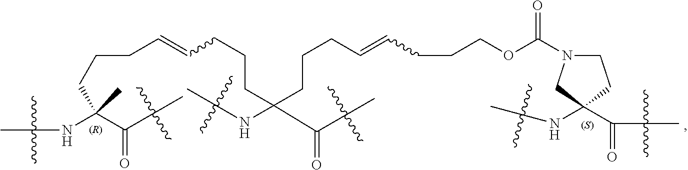

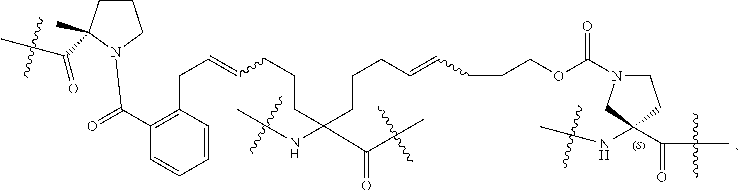

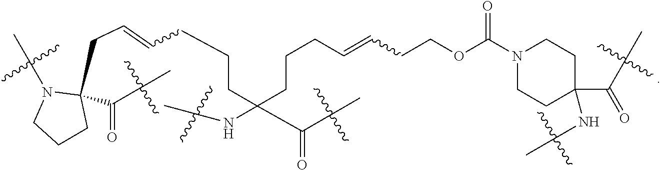 [SMILES: *NC(CCCC=CCCOC(=O)N1CCC(N*)(C(*)=O)CC1)(CCCC=CC[C@@]1(C(*)=O)CCCN1*)C(*)=O]

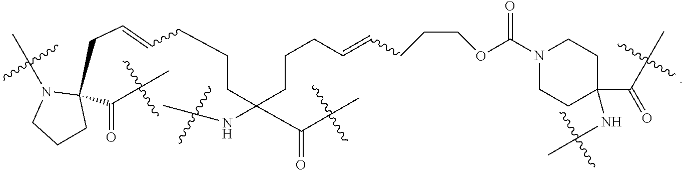 [SMILES: *NC(CCCC=CCCCOC(=O)N1CCC(N*)(C(*)=O)CC1)(CCCC=CC[C@@]1(C(*)=O)CCCN1*)C(*)=O]

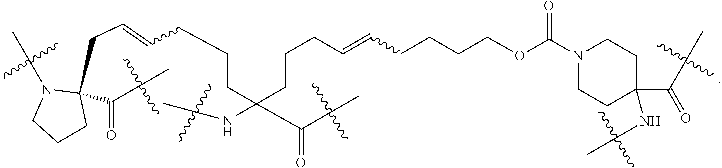 [SMILES: *NC(CCCC=CCCCCOC(=O)N1CCC(N*)(C(*)=O)CC1)(CCCC=CC[C@@]1(C(*)=O)CCCN1*)C(*)=O]

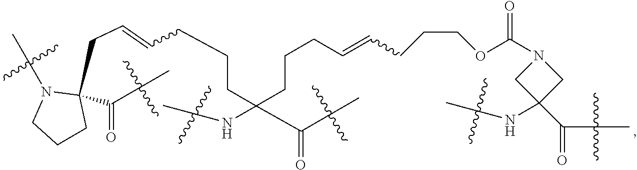 [SMILES: *NC(CCCC=CCCCOC(=O)N1CC(N*)(C(*)=O)C1)(CCCC=CC[C@@]1(C(*)=O)CCCN1*)C(*)=O]

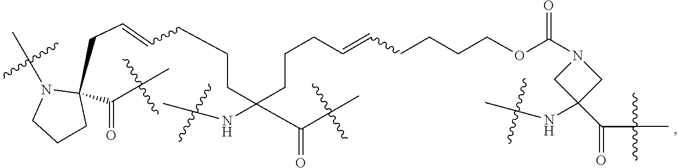 [SMILES: *NC(CCCC=CCCCCOC(=O)N1CC(N*)(C(*)=O)C1)(CCCC=CC[C@@]1(C(*)=O)CCCN1*)C(*)=O]

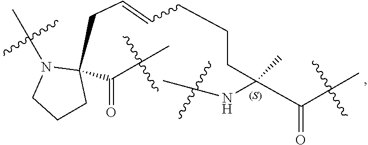

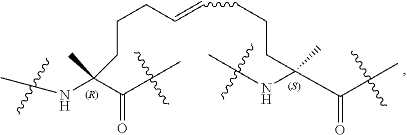

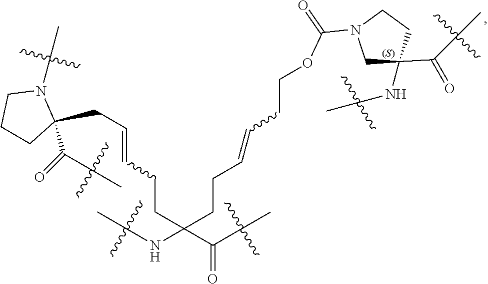

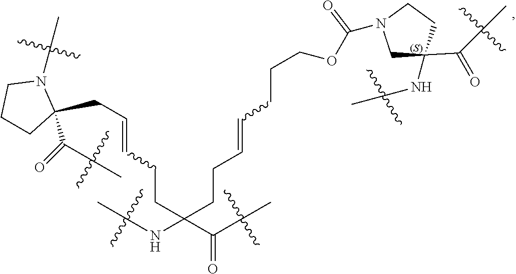

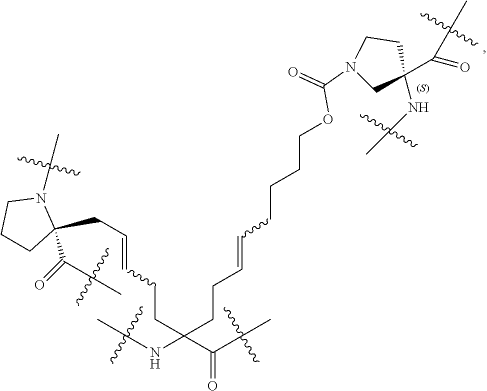

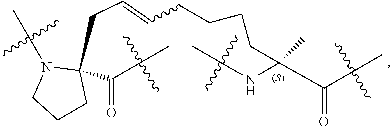

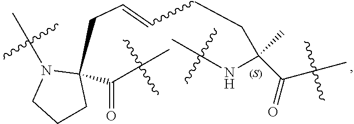

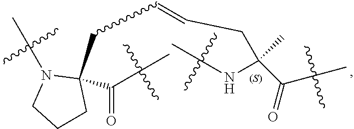

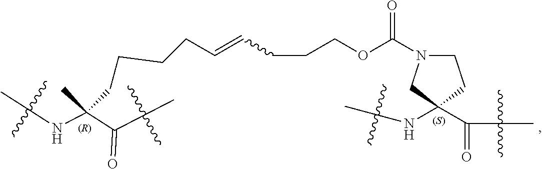

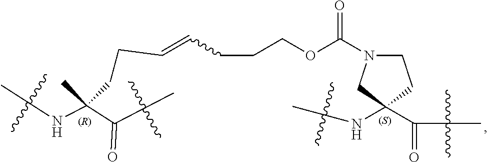

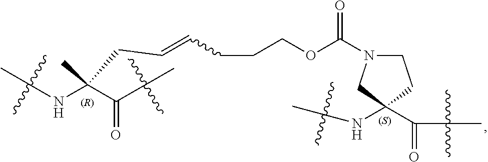

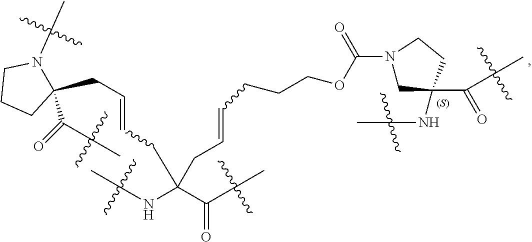

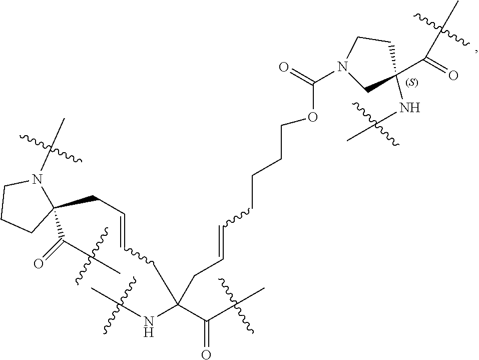

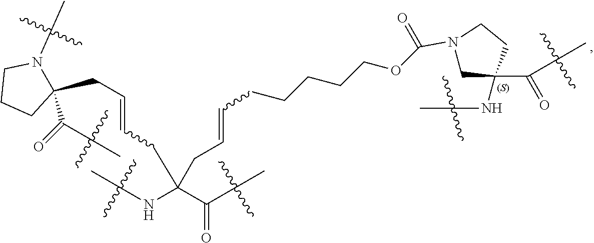

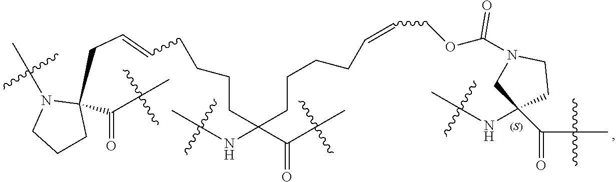

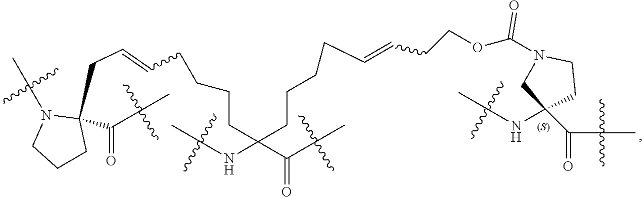

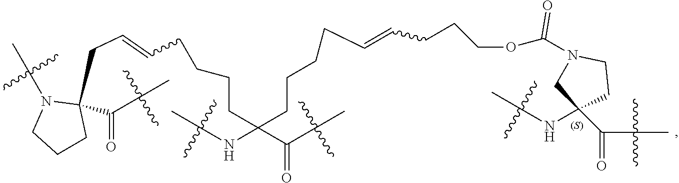

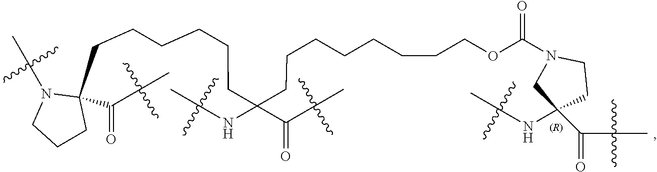

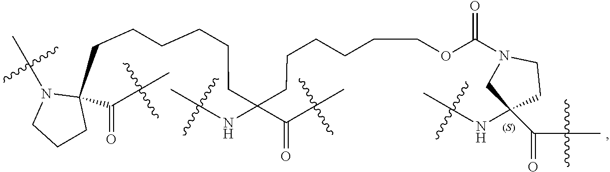

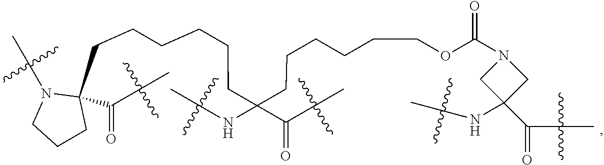 [SMILES: *NC(CCCCCCOC(=O)N1CC(N*)(C(*)=O)C1)(CCCCCC[C@@]1(C(*)=O)CCCN1*)C(*)=O]

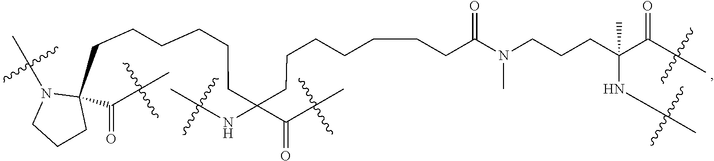 [SMILES: *NC(CCCCCCCC(=O)N(C)CCC[C@](C)(N*)C(*)=O)(CCCCCC[C@@]1(C(*)=O)CCCN1*)C(*)=O]

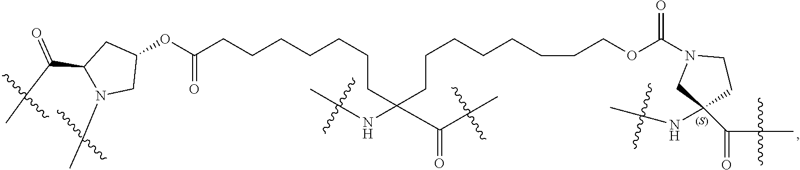

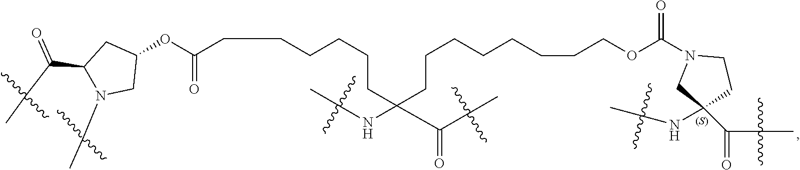

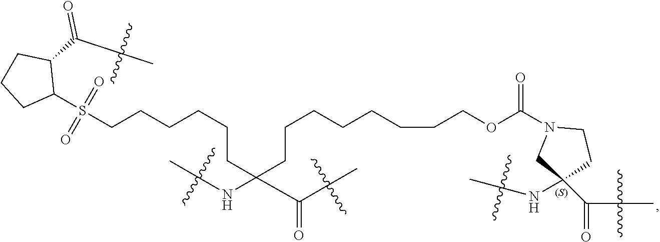

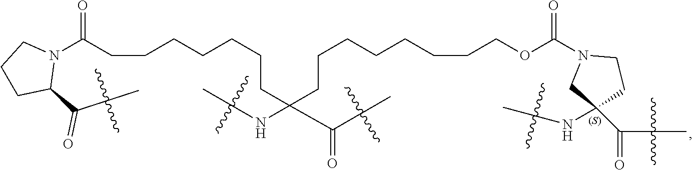

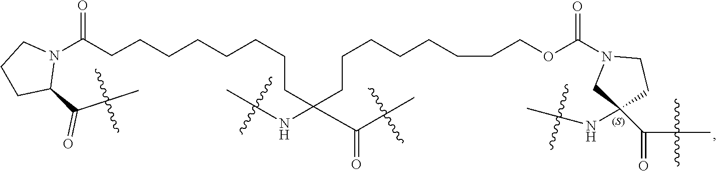

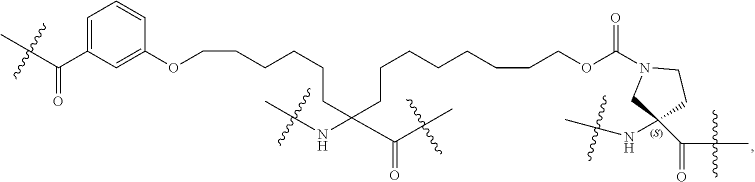

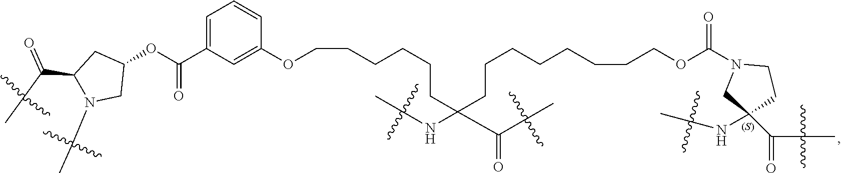

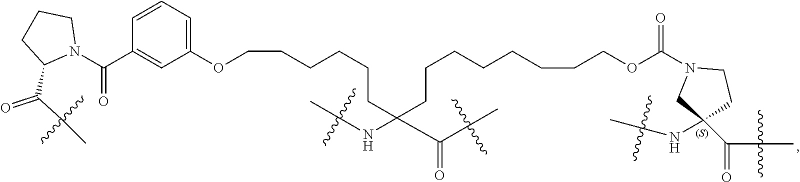

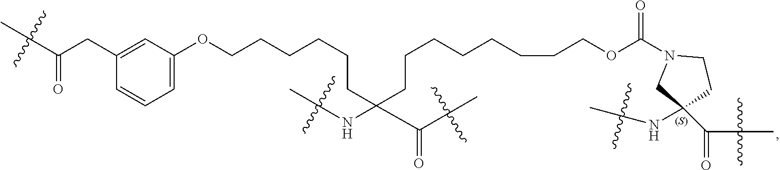

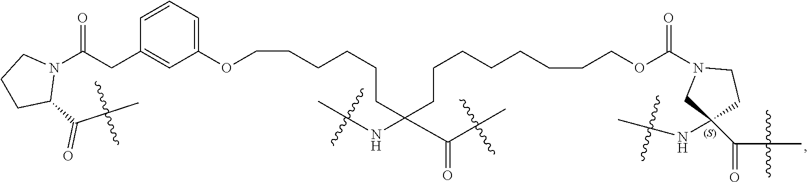

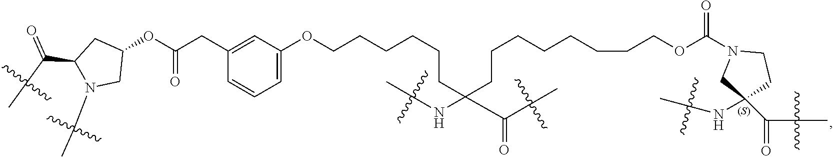

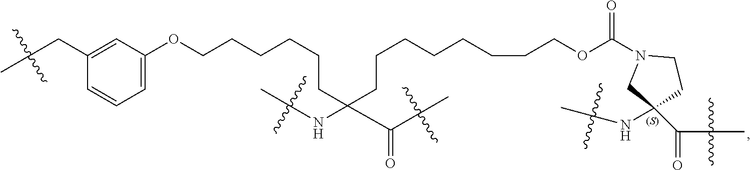

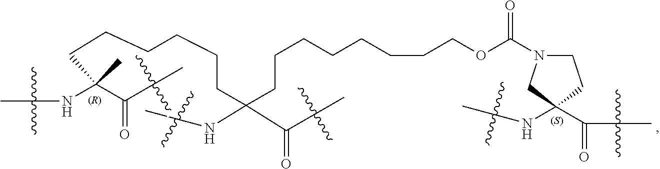

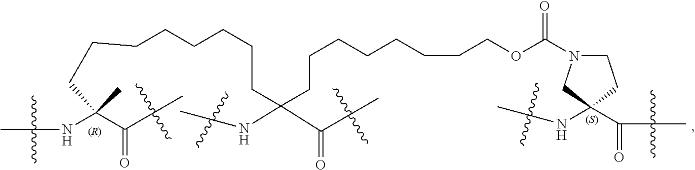

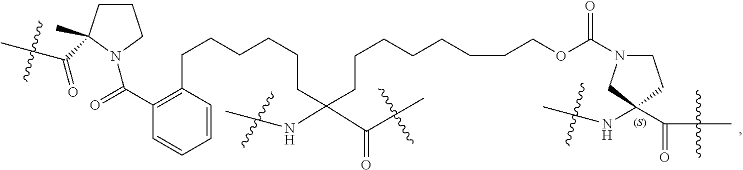

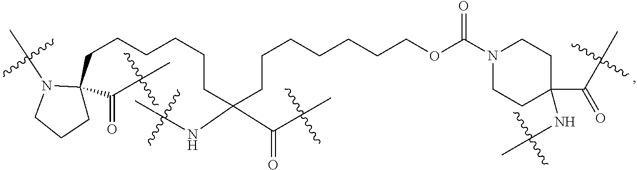 [SMILES: *NC(CCCCCCCOC(=O)N1CCC(N*)(C(*)=O)CC1)(CCCCCC[C@@]1(C(*)=O)CCCN1*)C(*)=O]

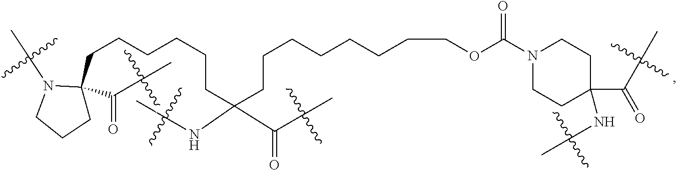 [SMILES: *NC(CCCCCCCCOC(=O)N1CCC(N*)(C(*)=O)CC1)(CCCCCC[C@@]1(C(*)=O)CCCN1*)C(*)=O]

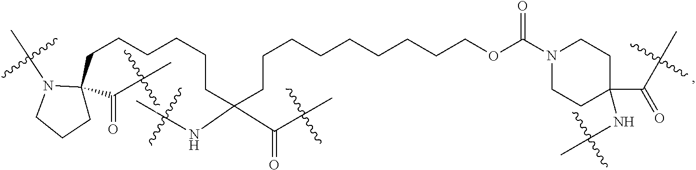 [SMILES: *NC(CCCCCCCCCOC(=O)N1CCC(N*)(C(*)=O)CC1)(CCCCCC[C@@]1(C(*)=O)CCCN1*)C(*)=O]

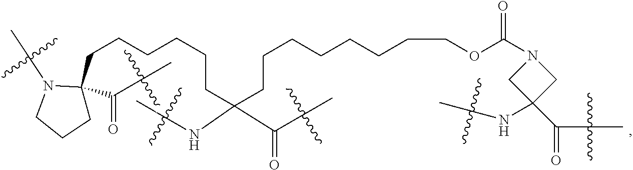 [SMILES: *NC(CCCCCCCCOC(=O)N1CC(N*)(C(*)=O)C1)(CCCCCC[C@@]1(C(*)=O)CCCN1*)C(*)=O]

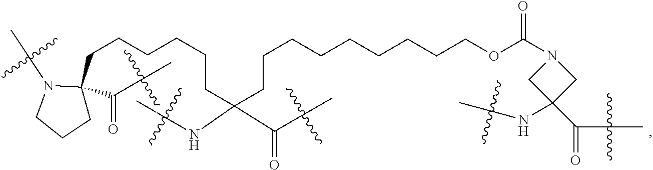 [SMILES: *NC(CCCCCCCCCOC(=O)N1CC(N*)(C(*)=O)C1)(CCCCCC[C@@]1(C(*)=O)CCCN1*)C(*)=O]

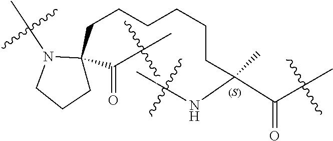

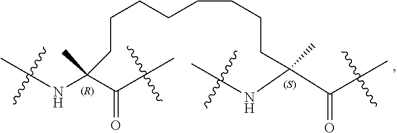

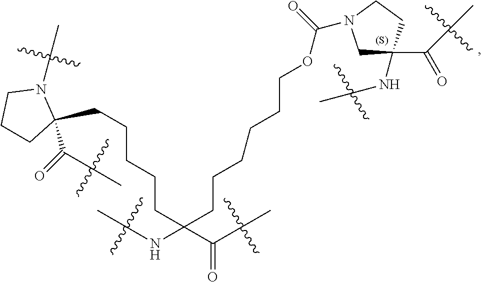

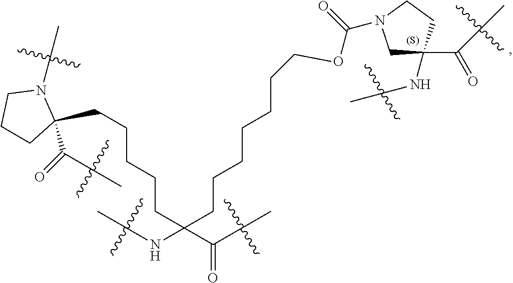

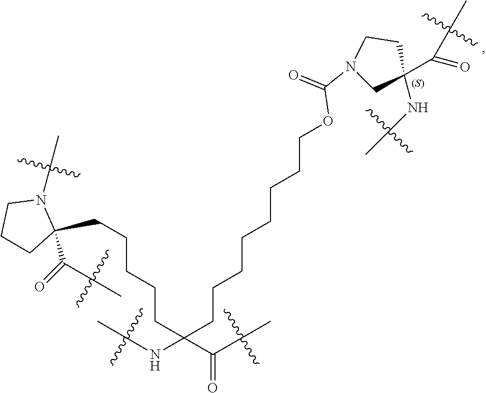

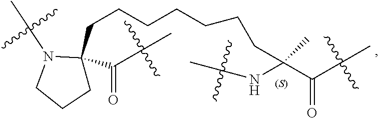

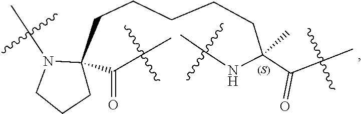

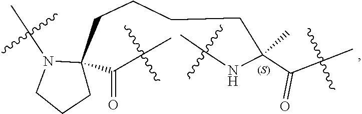

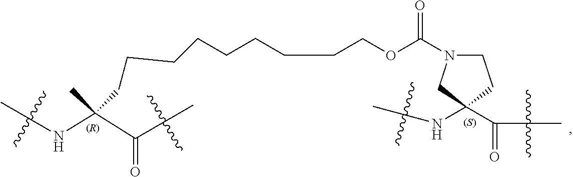

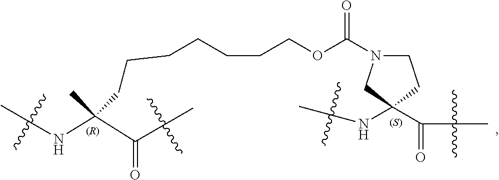

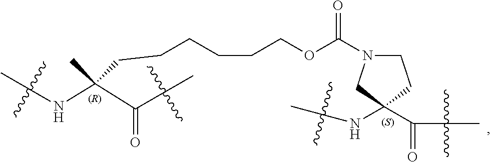

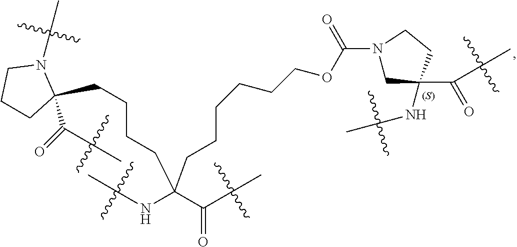

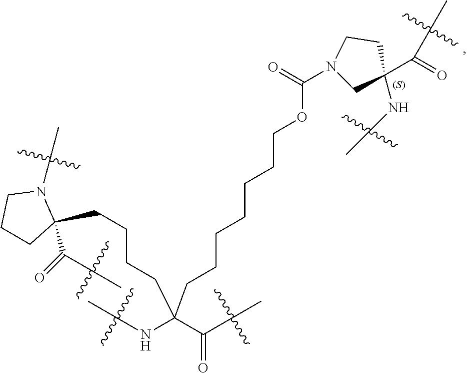

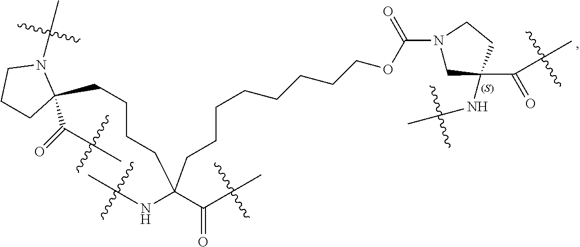

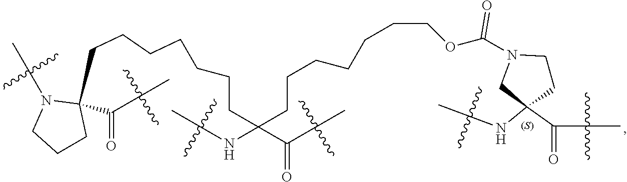

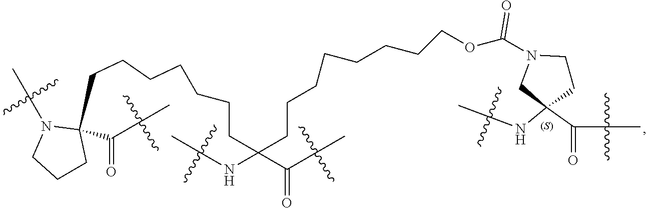

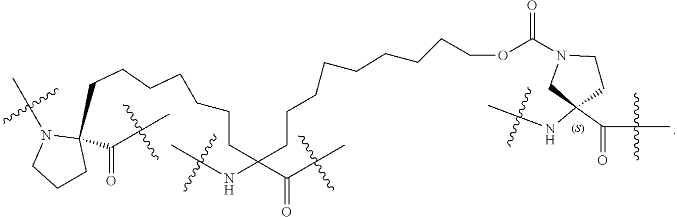

In some embodiments, the double bond in a (i, i+3) staple is Z. In some embodiments, the double bond in a (i, i+4) staple is Z. In some embodiments, the double bond in a (i, i+7) staple is Z. In some embodiments, the double bond in a (i, i+3) staple is E. In some embodiments, the double bond in a (i, i+4) staple is E. In some embodiments, the double bond in a (i, i+7) staple is E.

In some embodiments, a staple comprises —S—. In some embodiments, stapling technologies comprise utilization of one or more, e.g., two or more, sulfur-containing moieties. In some embodiments, a stapled peptide comprises cysteine stapling. In some embodiments, two cysteine residues are stapled wherein the —S— moieties of the two cysteine residues are connected optionally through a linker. In some embodiments, a stapled peptide comprises one and no more than one staples from cysteine stapling. In some embodiments, a stapled peptide comprises one and no more than one staples having the structure of

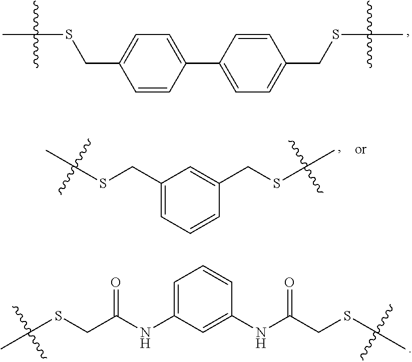

In some embodiments, a stapled peptide comprises one and no more than one staples having the structure of

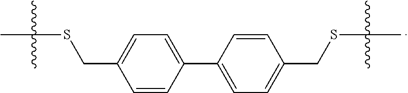 [SMILES: *SCc1ccc(-c2ccc(CS*)cc2)cc1]

In some embodiments, a stapled peptide comprises one and no more than one staples having the structure of

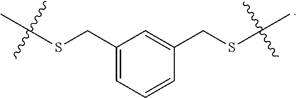 [SMILES: *SCc1cccc(CS*)c1]

In some embodiments, a stapled peptide comprises one and no more than one staples having the structure of

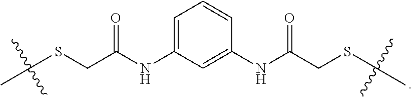 [SMILES: *SCC(=O)Nc1cccc(NC(=O)CS*)c1]

In some embodiments, a stapled peptide comprises no staples having the structure of

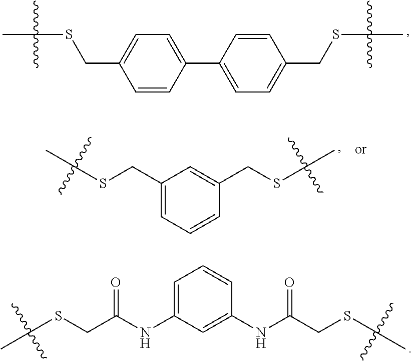

In some embodiments, a stapled peptide comprises no staples having the structure of

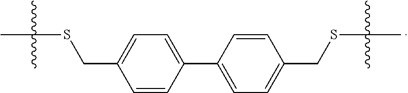 [SMILES: *SCc1ccc(-c2ccc(CS*)cc2)cc1]

In some embodiments, a stapled peptide comprises no staples having the structure of

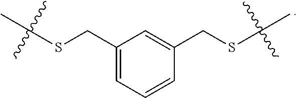 [SMILES: *SCc1cccc(CS*)c1]

In some embodiments, a stapled peptide comprises no staples having the structure of

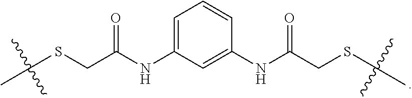 [SMILES: *SCC(=O)Nc1cccc(NC(=O)CS*)c1]

In some embodiments, the present disclosure provides useful technologies relating to cysteine stapling. Among other things, the present disclosure appreciates that peptides amenable to cysteine stapling and/or comprising one or more cysteine staples, can be produced and/or assessed in a biological system. The present disclosure further appreciates that certain such systems permit development, production, and/or assessment of cysteine stapled peptides having a range of different structures (e.g., different amino acid sequences), and in fact can provide a user with complete control over selection and implementation of amino acid sequences to be incorporated into stapled peptides.

Cysteine stapling, as described herein, involves linking one cysteine residue to another cysteine residue, where the resulting bond is not through the peptide backbone between the linked cysteine residues.

In some embodiments, a stapled peptide as described herein comprises a staple which staple is L^{s}, wherein:
L^{s} is -L^{s1}-S-L^{s2}-S-L^{s3}-;
L^{s1} and L^{s3} are each independently L;
L^{s2} is L and comprises at least one —C(O)—; and
each L is independently a covalent bond, or an optionally substituted, bivalent C₁-C₂₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—; each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆-30 arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L is independently a bivalent C₁-C₂₅ aliphatic group. In some embodiments, L is independently a bivalent C₁-C₂₀ aliphatic group. In some embodiments, L is independently a bivalent C₁-C₁₀ aliphatic group. In some embodiments, L is independently a bivalent C₁-C₅ aliphatic group. In some embodiments, L is independently a bivalent C₁ aliphatic group. In some embodiments, L is —CH₂.

In some embodiments, L^{s1} is —CH₂—. In some embodiments, L^{s3} is —CH₂—. In some embodiments, L^{s1} and L^{s3} are both —CH₂—. In some embodiments, L^{s} is —CH₂—S-L^{s2}-S—CH₂—.

In some embodiments, L^{s2} comprises —C(R′)₂-L′—C(R′)₂—, wherein L′ is described in the present disclosure. In some embodiments, L^{s2} is -L″-C(O)Q-L′-QC(O)-L″-, wherein each variable is independently as described in the present disclosure. In some embodiments, L^{s2} is —CH₂C(O)Q-L′-QC(O)CH₂—, wherein each —CH₂— is independently and optionally substituted. In some embodiments, L^{s2} is —CH₂C(O)Q-L′-QC(O)CH₂—.

In some embodiments, L^{s2} In some embodiments, L^{s2} is L and comprises at least one —C(O)—. In some embodiments, L^{s2} is L and comprises at least two —C(O)—. In some embodiments, L^{s2} is L and comprises at least one —C(O)Q-, wherein Q is selected from the group consisting of: a covalent bond, —N(R′)—, —O—, and —S—. In some embodiments, L^{s2} is L and comprises at least one —C(O)Q-, wherein Q is selected between —N(R′)— and —O—. In some embodiments, L^{s2} is L and comprises at least two —C(O)Q-, wherein Q is selected from the group consisting of: —N(R′)—, —O—, and —S—. In some embodiments, L^{s2} is L and comprises at least two —C(O)Q-, wherein Q is selected between —N(R′)— and —O—. In some embodiments, L^{s2} is L and comprises at least one —C(O)N(R′)—. In some embodiments, L^{s2} is L and comprises at least two —C(O)N(R′)—. In some embodiments, L^{s2} is L and comprises at least one —C(O)O—. In some embodiments, L^{s2} is L and comprises at least two —C(O)O—.

In some embodiments, L^{s2} comprises -Q-L′-Q-, wherein Q is independently selected from the group consisting of: —N(R′)—, —O—, and —S, wherein L′ is described in the present disclosure.

In some embodiments, L^{s2} comprises -Q-L′-Q-, wherein Q is independently selected between —N(R′)— and —O—, wherein L′ is described in the present disclosure. In some embodiments, L^{s2} comprises —C(O)Q-L′-QC(O)—, wherein Q is independently selected from the group consisting of: —N(R′)—, —O—, and —S, wherein L′ is described in the present disclosure. In some embodiments, L^{s2} comprises —C(O)Q-L′-QC(O)—, wherein Q is independently selected between —N(R′)— and —O, wherein L′ is described in the present disclosure. In some embodiments, L^{s2} comprises —C(R′)₂C(O)Q-L′-QC(O)C(R′)₂—, wherein Q is independently selected from the group consisting of: —N(R′)—, —O—, and —S, wherein L′ is described in the present disclosure. In some embodiments, L^{s2} comprises —C(R′)₂C(O)Q-L′-QC(O)C(R′)₂—, wherein Q is independently selected between —N(R′)— and —O, wherein L′ is described in the present disclosure.

In some embodiments, L^{s2} comprises —N(R′)-L′-N(R′)—, wherein L′ is described in the present disclosure. In some embodiments, L^{s2} comprises —C(O)N(R′)-L′-N(R′)C(O)—, wherein L′ is described in the present disclosure. In some embodiments, L^{s2} is —C(R′)₂C(O)N(R′)-L′-N(R′)C(O)C(R′)₂—, wherein L′ is described in the present disclosure.

In some embodiments, L^{s2} comprises —O(R′)-L′-O(R′)—, wherein L′ is described in the present disclosure. In some embodiments, L^{s2} comprises —C(O)O-L′-OC(O)—, wherein L′ is described in the present disclosure. In some embodiments, L^{s2} is —C(R′)₂C(O)O-L′-OC(O)C(R′)₂—, wherein L′ is described in the present disclosure.

In some embodiments, R′ is an optionally substituted C₁₋₃₀ aliphatic. In some embodiments, R′ is an optionally substituted C₁₋₁₅ aliphatic. In some embodiments, R′ is an optionally substituted C₁₋₁₀ aliphatic. In some embodiments, R′ is an optionally substituted C₁₋₅ aliphatic. In some embodiments, R′ is hydrogen.

In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₈ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₃ aliphatic. In some embodiments, L′ is optionally substituted bivalent C₁-C₂ aliphatic. In some embodiments, L′ is optionally substituted bivalent C₁ aliphatic. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—.

In some embodiments, L′ is optionally substituted bivalent C₆₋₂₀ aryl ring. In some embodiments, L′ is optionally substituted bivalent C₆₋₁₄ aryl ring. In some embodiments, L′ is optionally substituted bivalent C₆₋₁₀ aryl ring. In some embodiments, L′ is optionally substituted bivalent C₆ aryl ring. In some embodiments, L′ is bivalent C₆ aryl substituted with at least one halogen. In some embodiments, L′ is bivalent C₆ aryl substituted with at least two halogen. In some embodiments, L′ is bivalent C₆ aryl substituted with at least three halogen. In some embodiments, L′ is bivalent C₆ aryl substituted with four halogen. In some embodiments, L′ is bivalent C₆ aryl substituted with at least one fluorine. In some embodiments, L′ is bivalent C₆ aryl substituted with at least two fluorine. In some embodiments, L′ is bivalent C₆ aryl substituted with at least three fluorine. In some embodiments, L′ is bivalent C₆ aryl substituted with four fluorine. In some embodiments, L′ is bivalent C₆ aryl substituted with at least one chlorine. In some embodiments, L′ is bivalent C₆ aryl substituted with at least two chlorine. In some embodiments, L′ is bivalent C₆ aryl substituted with at least three chlorine. In some embodiments, L′ is bivalent C₆ aryl substituted with four chlorine. In some embodiments, L′ is bivalent C₆ aryl substituted at with least one —O(CH₂)₀₋₄CH₃. In some embodiments, L′ is bivalent C₆ aryl substituted with at least two —O(CH₂)₀₋₄CH₃. In some embodiments, L′ is bivalent C₆ aryl substituted with at least three —O(CH₂)₀₋₄CH₃. In some embodiments, L′ is bivalent C₆ aryl substituted with four —O(CH₂)₀₋₄CH₃.

In some embodiments, L′ is bivalent 5-20 membered heteroaryl ring having 1-10 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, and sulfur. In some embodiments, L′ is bivalent 6 membered heteroaryl ring having 1-2 heteroatoms independently selected from oxygen, nitrogen, and sulfur. In some embodiments, L′ is bivalent 6 membered heteroaryl ring having 2 nitrogen.

In some embodiments, L′ is optionally substituted bivalent C₃₋₂₀ cycloaliphatic ring. In some embodiments, L′ is optionally substituted bivalent C₃₋₁₅ cycloaliphatic ring. In some embodiments, L′ is optionally substituted bivalent C₃₋₁₀ cycloaliphatic ring. In some embodiments, L′ is optionally substituted bivalent C₃₋₉ cycloaliphatic ring. In some embodiments, L′ is optionally substituted bivalent C₃₋₈ cycloaliphatic ring. In some embodiments, L′ is optionally substituted bivalent C₃₋₇ cycloaliphatic ring. In some embodiments, L′ is optionally substituted bivalent C₃₋₆ cycloaliphatic ring. In some embodiments, L′ is optionally substituted bivalent C₃₋₅ cycloaliphatic ring. In some embodiments, L′ is optionally substituted bivalent C₃₋₄ cycloaliphatic ring. In some embodiments, L′ is optionally substituted bivalent C₃ cycloaliphatic ring. In some embodiments, L′ is optionally substituted bivalent C₄ cycloaliphatic ring. In some embodiments, L′ is optionally substituted bivalent C₅ cycloaliphatic ring. In some embodiments, L′ is optionally substituted bivalent C₅ cycloalkyl ring. In some embodiments, L′ is optionally substituted bivalent C₅ cycloalkenyl ring. In some embodiments, L′ is optionally substituted bivalent C₆ cycloaliphatic ring. In some embodiments, L′ is optionally substituted bivalent C₆ cycloalkyl ring.

In some embodiments, L^{s2} comprises —N(R′)-L′-N(R′)— and L′ is a covalent bond. In some embodiments L^{s2} comprises —N(R)—N(R)—, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆-30 aryl, C₆-30 arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments L^{s2} comprises —N(R)—N(R)—, wherein:
each R is independently optionally substituted C₁₋₃₀ aliphatic; or
two or more R groups on two or more atoms are optionally and independently taken together with their intervening atoms to form an optionally substituted, 3-30 membered monocyclic ring.

In some embodiments, L^{s2} is a staple selected from the group consisting of

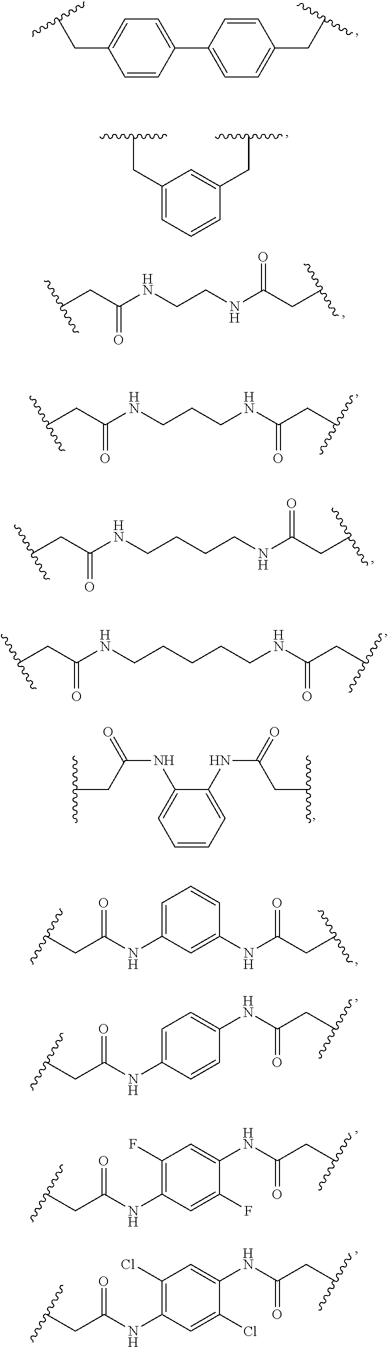

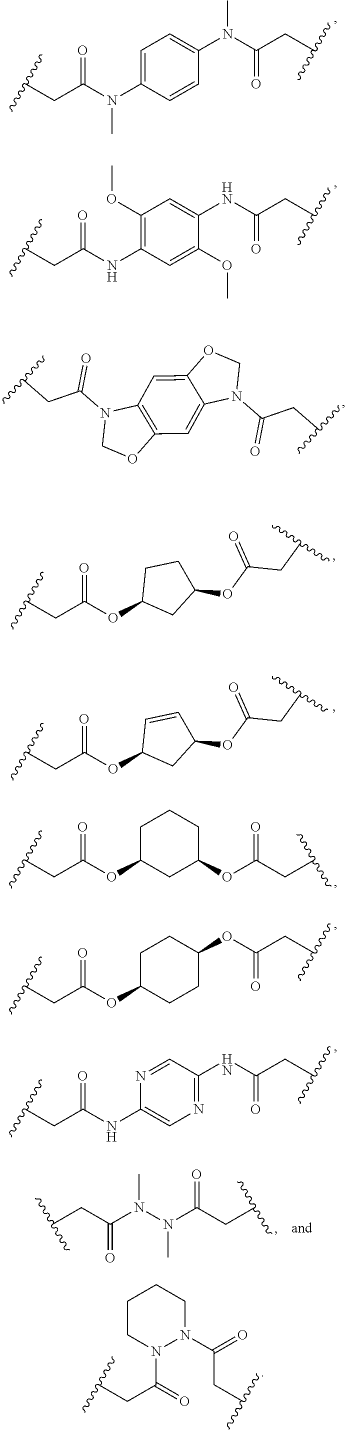

In some embodiments, L^{s1} is optionally substituted bivalent C₁₋₆ aliphatic. In some embodiments, L^{s} is bivalent C₁₋₆ aliphatic. In some embodiments, L^{s} is bivalent C₁₋₄ aliphatic. In some embodiments, L^{s1} is saturated. In some embodiments, L^{s1} is linear. In some embodiments, L^{s1} is branched. In some embodiments, L^{s1} is optionally substituted —CH₂—. In some embodiments, L^{s1} is —CH₂—. In some embodiments, L^{s1} is optionally substituted —CH₂—CH₂—. In some embodiments, L^{s1} is —CH₂—CH₂—. In some embodiments, L^{s1} is optionally substituted —C(CH₃)₂—. In some embodiments, L^{s1} is —C(CH₃)₂—.

In some embodiments, L^{s2} is optionally substituted bivalent C₁₋₆, (e.g., C₃₋₆, C₃, C₄, C₅, C₆, etc.) aliphatic wherein one or more methylene units are optionally and independently replaced with -Cy- or —C(R′)₂—. In some embodiments, L^{s2} is optionally substituted bivalent C₁₋₆ aliphatic. In some embodiments, L^{s2} is optionally substituted bivalent C₃₋₆ aliphatic. In some embodiments, L^{s2} is bivalent C₁₋₆ aliphatic. In some embodiments, L^{s2} is bivalent C₁₋₄ aliphatic. In some embodiments, L^{s2} is optionally substituted bivalent C₂ aliphatic. In some embodiments, L^{s2} is optionally substituted bivalent C₃ aliphatic. In some embodiments, L^{s2} is optionally substituted bivalent C₄ aliphatic. In some embodiments, L^{s2} is optionally substituted bivalent C₅ aliphatic. In some embodiments, L^{s2} is optionally substituted bivalent C₆ aliphatic. In some embodiments, L^{s2} is substituted. In some embodiments, L^{s2} is unsubstituted. In some embodiments, L^{s2} is saturated. In some embodiments, L^{s2} is linear. In some embodiments, L^{s2} is branched. In some embodiments, L^{s2} is optionally substituted bivalent C₃₋₆, (e.g., C₃₋₅, C₃, C₄, C₅, C₆, etc.) aliphatic wherein one or two methylene units are independently replaced with -Cy-. In some embodiments, L^{s2} is —CH₂-Cy-CH₂—. In some embodiments, L^{s2} is —CH₂—CH₂-Cy-CH₂—CH₂—. In some embodiments, L^{s2} is —CH₂-Cy-Cy-CH₂—. Various useful embodiments of -Cy- are as described herein. For example, in some embodiments, -Cy- is an optionally substituted monocyclic 5-membered aromatic ring having 0-4 heteroatoms. In some embodiments, -Cy- is an optionally substituted monocyclic 6-membered aromatic ring having 0-4 heteroatoms. In some embodiments, -Cy- is optionally substituted phenylene. In some embodiments, -Cy- is optionally substituted 1,2-phenylene. In some embodiments, -Cy- is 1,2-phenylene. In some embodiments, -Cy- is optionally substituted 1,3-phenylene. In some embodiments, -Cy- is 1,3-phenylene. In some embodiments, -Cy- is optionally substituted 1,5-phenylene. In some embodiments, -Cy- is 1,5-phenylene. In some embodiments, -Cy- is 3-methyl-1,5-phenylene. In some embodiments, -Cy- is 3-methoxy-1,5-phenylene. In some embodiments, -Cy- is an optionally substituted bivalent pyridyl ring. In some embodiments, -Cy- is optionally substituted

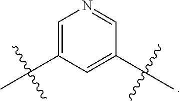 [SMILES: *c1cncc(*)c1]

In some embodiments, -Cy- is

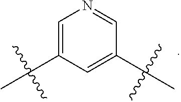 [SMILES: *c1cncc(*)c1]

In some embodiments, -Cy- is optionally substituted

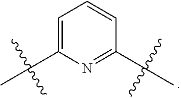 [SMILES: *c1cccc(*)n1]

In some embodiments, -Cy- is

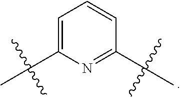 [SMILES: *c1cccc(*)n1]

In some embodiments, -Cy- is optionally substituted bicyclic 9-membered aromatic ring having 0-4 heteroatoms. In some embodiments, -Cy- is optionally substituted bicyclic 10-membered aromatic ring having 0-4 heteroatoms. In some embodiments, -Cy- is optionally substituted bivalent naphthyl ring. In some embodiments, -Cy- is a bivalent naphthyl ring. In some embodiments, -Cy- is optionally substituted

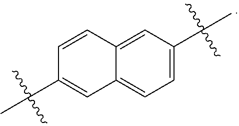 [SMILES: *c1ccc2cc(*)ccc2c1]

In some embodiments, -Cy- is

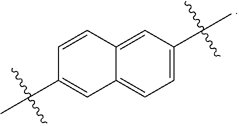 [SMILES: *c1ccc2cc(*)ccc2c1]

In some embodiments, -Cy- is optionally substituted

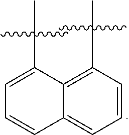 [SMILES: *c1cccc2cccc(*)c12]

In some embodiments, -Cy- is

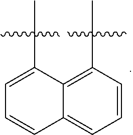 [SMILES: *c1cccc2cccc(*)c12]

In some embodiments, -Cy- is optionally substituted 3-10 (e.g., 5-10, 5-6, 3, 4, 5, 6, 7, 8, 9, 10, etc.) membered bivalent cycloaliphatic ring. In some embodiments, it is saturated. In some embodiments, -Cy- is an optionally substituted 6-membered cycloalkyl ring. In some embodiments, -Cy- is optionally substituted

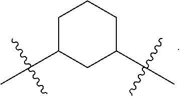 [SMILES: *C1CCCC(*)C1]

In some embodiments, -Cy- is

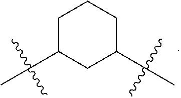 [SMILES: *C1CCCC(*)C1]

In some embodiments, L^{s2} is optionally substituted bivalent C₃₋₆, (e.g., C₃₋₅, C₃, C₄, C₅, C₆, etc.) aliphatic wherein one or two methylene units are independently replaced with —C(R′)₂—. In some embodiments, L^{s2} is —CH₂—C(R′)₂—CH₂—. In some embodiments, the two R′ are taken together with the carbon atom to form an optionally substituted ring as described herein, e.g., an optionally substituted 3-10 (e.g., 5-10, 5-6, 3, 4, 5, 6, 7, 8, 9, 10, etc.) membered ring having 0-4 (e.g., 1-4, 0, 1, 2, 3, 4, etc.) heteroatoms. In some embodiments, a ring is saturated. In some embodiments, a ring has one or more heteroatoms. In some embodiments, —C(R′)₂— is

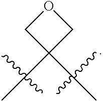 [SMILES: *C1(*)COC1]

In some embodiments, L^{s2} is optionally substituted

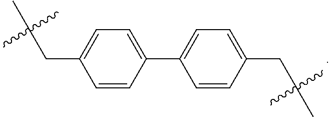 [SMILES: *Cc1ccc(-c2ccc(C*)cc2)cc1]

In some embodiments, L^{s2} is optionally substituted

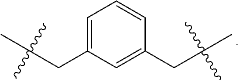 [SMILES: *Cc1cccc(C*)c1]

In some embodiments, L^{s2} is optionally substituted

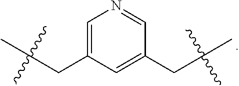 [SMILES: *Cc1cncc(C*)c1]

In some embodiments, L^{s2} is optionally substituted

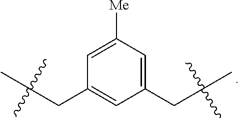 [SMILES: *Cc1cc(C)cc(C*)c1]

In some embodiments, L^{s2} is optionally substituted

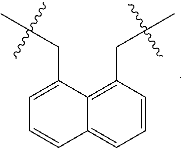 [SMILES: *Cc1cccc2cccc(C*)c12]

In some embodiments, L^{s2} is optionally substituted

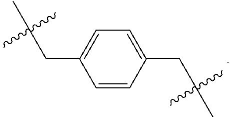 [SMILES: *Cc1ccc(C*)cc1]

In some embodiments, L^{s2} is optionally substituted

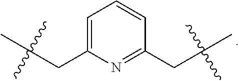 [SMILES: *Cc1cccc(C*)n1]

In some embodiments, L^{s2} is optionally substituted

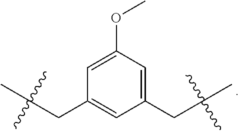 [SMILES: *Cc1cc(C*)cc(OC)c1]

In some embodiments, L^{s2} is optionally substituted

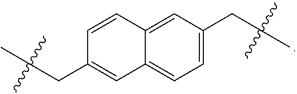 [SMILES: *Cc1ccc2cc(C*)ccc2c1]

In some embodiments, L^{s} is optionally substituted

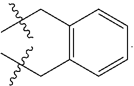 [SMILES: *Cc1ccccc1C*]

In some embodiments, L^{s2} is optionally substituted —(CH₂)₄—. In some embodiments, L^{s2} is optionally substituted —(CH₂)₃—. In some embodiments, L^{s2} is optionally substituted —CH₂—CH═CH—CH₂—. In some embodiments, L^{s2} is optionally substituted (E)-CH₂—CH═CH—CH₂—. In some embodiments, L^{s2} is optionally substituted —CH₂—C(O)—CH₂—. In some embodiments, L^{s2} is optionally substituted

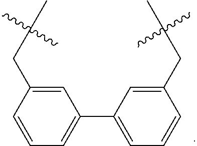 [SMILES: *Cc1cccc(-c2cccc(C*)c2)c1]

In some embodiments, L^{s2} is optionally substituted

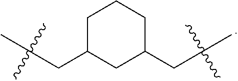 [SMILES: *CC1CCCC(C*)C1]

In some embodiments, L^{s2} is optionally substituted

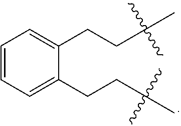 [SMILES: *CCc1ccccc1CC*]

In some embodiments, L^{s2} is optionally substituted

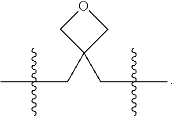 [SMILES: *CC1(C*)COC1]

In some embodiments, it is substituted. In some embodiments, it is unsubstituted. In some embodiments,

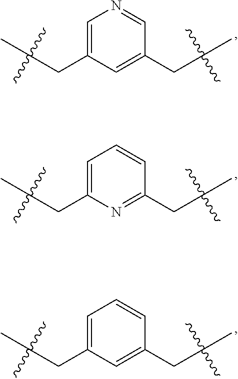 [SMILES: *Cc1cncc(C*)c1 ; *Cc1cccc(C*)n1 ; *Cc1cccc(C*)c1]

—(CH₂)₄—, (E)-CH₂—CH═CH—CH₂—, —(CH₂)₃—, and/or —CH₂—C(O)—CH₂— provide higher binding and/or potency than

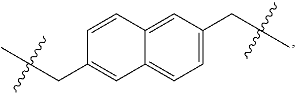 [SMILES: *Cc1ccc2cc(C*)ccc2c1]

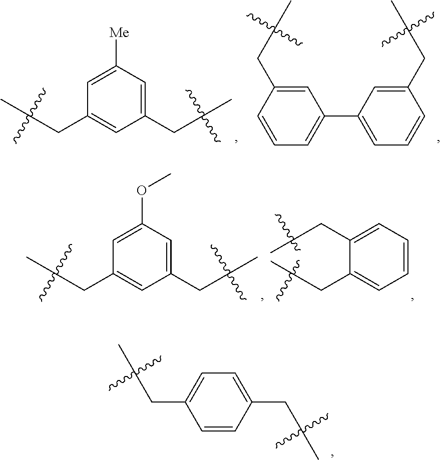

and/or

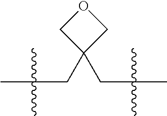 [SMILES: *CC1(C*)COC1]

under comparable conditions.

In some embodiments, L^{s3} is optionally substituted bivalent C₁₋₆ aliphatic. In some embodiments, L^{s3} is bivalent C₁₋₆ aliphatic. In some embodiments, L^{s3} is bivalent C₁₋₄ aliphatic. In some embodiments, L^{s3} is saturated. In some embodiments, L^{s3} is linear. In some embodiments, L^{s3} is branched. In some embodiments, L^{s3} is optionally substituted —CH₂—. In some embodiments, L^{s3} is —CH₂—. In some embodiments, L^{s3} is optionally substituted —CH₂—CH₂—. In some embodiments, L^{s3} is —CH₂—CH₂—. In some embodiments, L^{s3} is optionally substituted —C(CH₃)₂—. In some embodiments, L^{s3} is —C(CH₃)₂—.

In some embodiments, an amino acid residue for forming a staple is selected from:

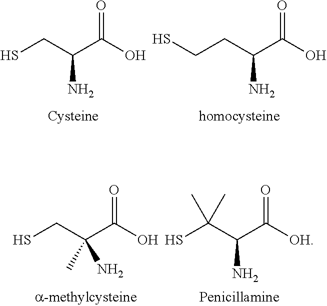

In some embodiments, both amino acid residue for forming a staple are independently residues of these amino acids. In some embodiments, each of L^{s1} and L^{s3} is independently —CH₂—, —CH₂—CH₂—, or —C(CH₃)₂—. In some embodiments, a staple is formed by reacting the thiol groups with a thiol reactive linker compound. In some embodiments, such a linker compound has the structure of LG-L^{s2}-LG or a salt thereof, wherein each LG is independently a leaving group, e.g., —Br, —I, etc. In some embodiments, each LG is independently —Br or —I. In some embodiments, each LG is —Br. In some embodiments, each LG is —I. In some embodiments, L^{s2} are of such structures that LG-L^{s2}-LG (each LG is independently —Br or —I) is a compound selected from:

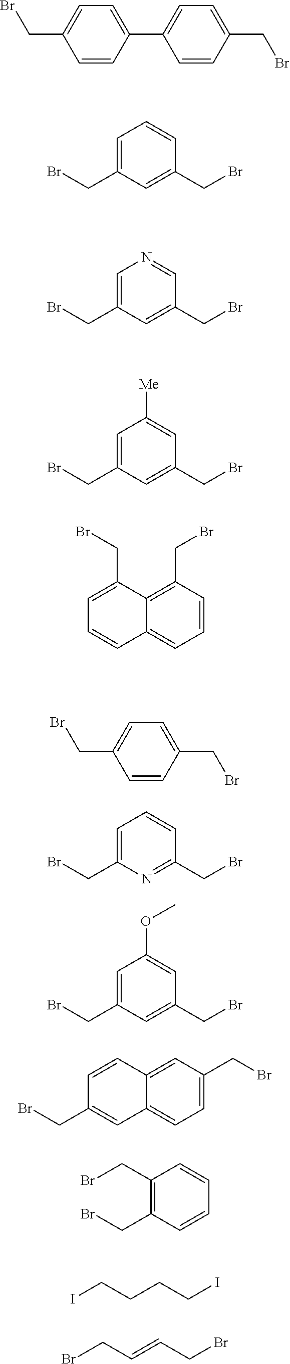

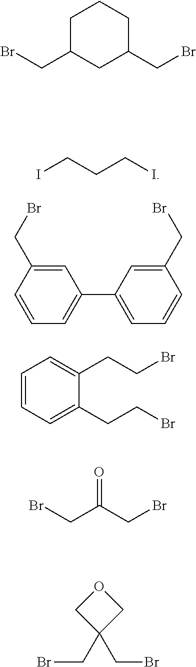

Various technologies are available for constructing of thioether staples. For example, in some embodiments, a peptide and excess equivalents (e.g., about 2-10, 5-10, 2, 3, 4, 5, 6, 7, 8, 9, 10, etc.; in some embodiments, 5) of a linker compound were added to a 1:1 DMF:100 mM Na₂CO₃ pH 8.0 solution and stirred at a suitable temperature, e.g., room temperature for a suitable period of time, in some embodiments, 1-2 hours. In some embodiments, e.g., for relatively weaker electrophiles, excess equivalents (e.g., about 10-30, 10-20, 10, 20, etc.; in some embodiments, 20) of a metal salt, e.g., Zn(acac)₂ and an excess equivalents (e.g., about 5-20, 10-15, 10, 15, 20, etc.; in some embodiments, 10-15) of a linker compound were added to a peptide in DMA, and the mixture was stirred for a suitable period of time, e.g., overnight, at a suitable temperature, e.g., 37° C. In some embodiments, equivalents of Zn(acac)₂ and linker compounds were doubled, and/or the temperature was increased to 50° C. In some embodiments, certain linker compounds react better than others. For example, in some embodiments,

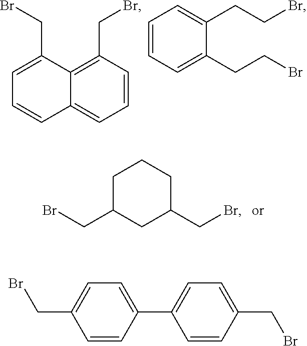

Br provides poor reaction yields or failed reactions. Those skilled in the art appreciate that other technologies may be utilized to introduce the corresponding linker moieties (L^{s2}), e.g., through utilizing other leaving groups or through other reaction mechanisms/routes.

In some embodiments, a staple having the structure of -L^{s1}-S-L^{s2}-S-L^{s3}- is a (i, i+4) staple. In some embodiments, such a staple is in closer to a C-terminus. In some embodiments, such a staple is in closer to a N-terminus. For example, in some embodiments, such a staple is between X¹⁰ and X¹⁴.

In some embodiments, certain staples provide better properties and/or activities. For example, in some embodiments, based on target binding affinity certain staples/scaffolds is ranked in the following order:

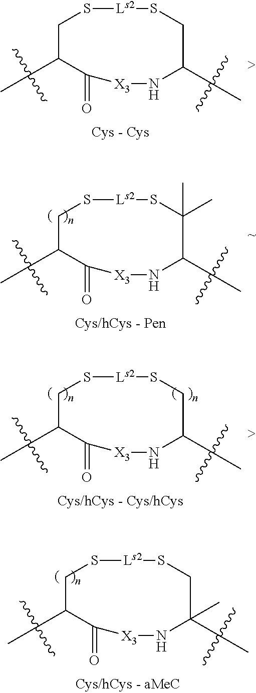

As those skilled in the art will appreciate, provided technologies can be utilized to prepare collection of peptides using non-cysteine residues and suitable chemistry therefor. For example, in some embodiments, cysteine stapling is replaced with lysine stapling, wherein the cysteine residues for cysteine stapling are replaced with lysine residues for lysine stapling (e.g., using agents that can crosslink two lysine residues, for example, through reactions with side chain amino groups). In some embodiments, for lysine stapling, R^{E} in various formulae is or comprises an activated carboxylic acid group (e.g., NHS ester group), an imidoester group, etc. Suitable reagents are widely known in the art including many commercially available ones. In some embodiments, cysteine stapling is replaced with methionine stapling. In some embodiments, cysteine residues for cysteine stapling are replaced with methionine residues for methionine stapling. In some embodiments, cysteine stapling is replaced with tryptophan stapling. In some embodiments, cysteine residues for cysteine stapling are replaced with tryptophan residues for tryptophan stapling. As those skilled in the art will appreciate, various technologies (e.g., reagents, reactions, etc.) are described in the art and can be utilized in accordance with the present disclosure for, e.g., methionine stapling, tryptophan stapling, etc. In some embodiments, such stapling can be performed using reagents having various formulae described herein, wherein R^{E} is or comprises a group that are suitable for methionine and/or tryptophan stapling. In some embodiments, stapling may be performed using one residue at a first position, and a different residue at a second position. Useful reagents for such stapling may comprise a first reactive group for stapling at a first position (e.g., through a first R^{E}), and a second reactive group for stapling at a second position (e.g., through a second R^{E}).

In some embodiments, for various types of stapling (e.g., cysteine stapling, or non-cysteine stapling), stapling is between residues (e.g., cysteine residues for cysteine stapling) separated by two residues (i+3 stapling). In some embodiments, stapling is between residues separated by three residues (i+4 stapling). In some embodiments, stapling is between residues separated by six residues (i+7 stapling).

As appreciated by those skilled in the art, in some embodiments, more than two residues can be stapled at the same time. For example, in some embodiments, three or more cysteines are stapled using crosslinking reagents containing three or more reactive groups (e.g., R^{E} groups).

In some embodiments, as described herein, the present disclosure provides useful technologies relating to non-cysteine stapling. Among other things, the present disclosure appreciates that peptides amenable to cysteine stapling and/or comprising one or more non-cysteine staples, can have its cysteine residues and cysteine staple replaced with other amino acids and staples described herein (e.g. hydrocarbon and other non-hydrocarbon amino acid and staples). In some embodiments, the resulting non-cysteine stapled peptide maintains the same or similar interaction with a target of interest when compared to a reference cysteine stapled peptide.

Certain useful agents (peptides prior to stapling and stapled peptides post stapling) and compositions thereof are presented in Table E2 or Table E3 as examples, which includes various amino acid residues and N- and C-terminus capping groups for various positions as examples; also illustrated are various stapling patterns, e.g., X¹—X⁴—X¹¹, X¹—X³, X³—X⁷, X³—X¹⁰, X⁴—X¹¹, X⁷—X¹⁰, X⁷—X¹⁴, X¹⁰—X¹⁴, etc. As demonstrated herein, provided technologies can deliver improved useful properties and/or activities.

In some embodiments, a provided agent, a peptide, or a stapled peptide is a compound as described herein. In some embodiments, a provided agent has a structure selected from Table E2 or Table E3, or a salt thereof. In some embodiments, a provided agent is a stereoisomer of a structure selected from Table E2 or Table E3, or a salt thereof. In some embodiments, a provided agent is a stereoisomer, with respect to a chiral center bonded to two staples (e.g., in B4, B5, etc.), of a structure selected from Table E2 or Table E3, or a salt thereof. In some embodiments, a provided agent is a stereoisomer, with respect to olefin double bond(s) in staple(s), of a structure selected from Table E2 or Table E3, or a salt thereof. In some embodiments, a provided agent is a stereoisomer, with respect to olefin double bond(s) in staple(s) and/or a chiral center bonded to two staples (e.g., in B4, B5, etc.), of a structure selected from Table E2 or Table E3, or a salt thereof. In some embodiments, a provided composition is a composition described in Table E2 or Table E3. In some embodiments, a compound has the structure of

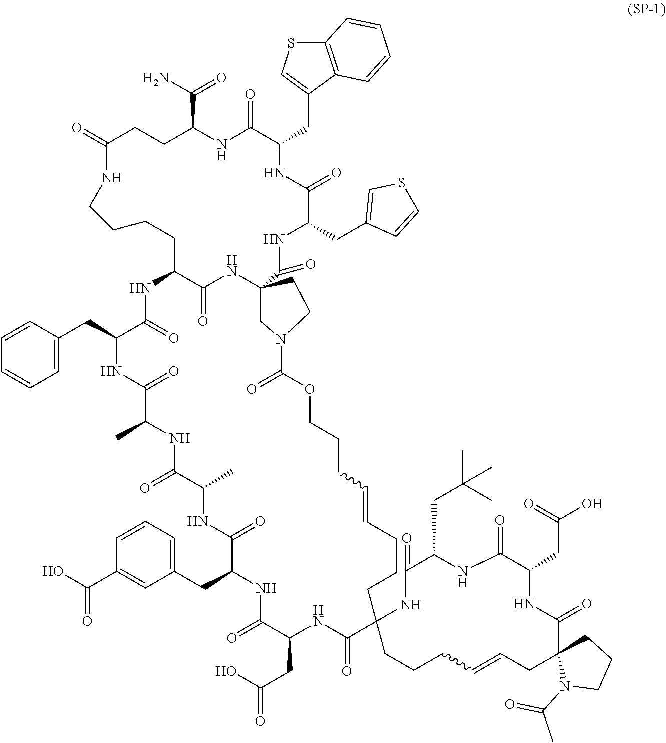 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)[C@H](C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

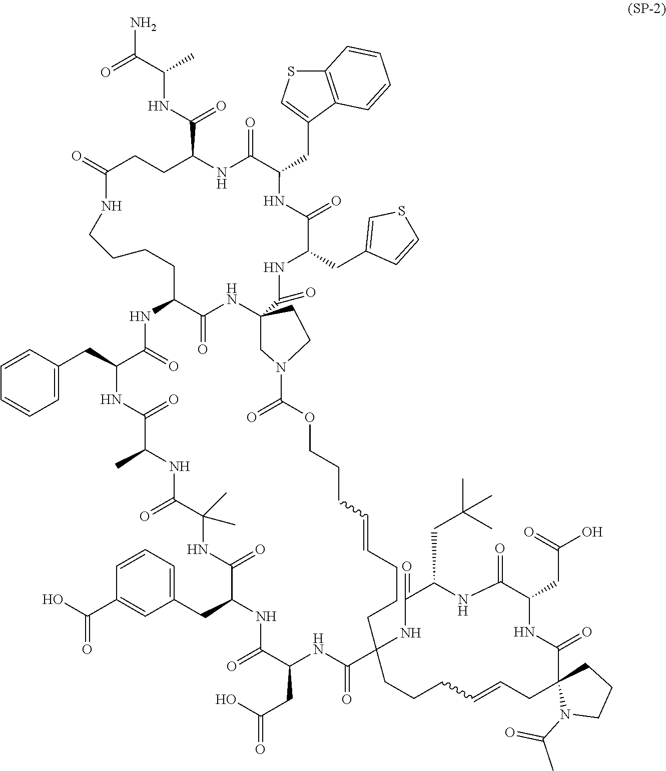 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has a structure of

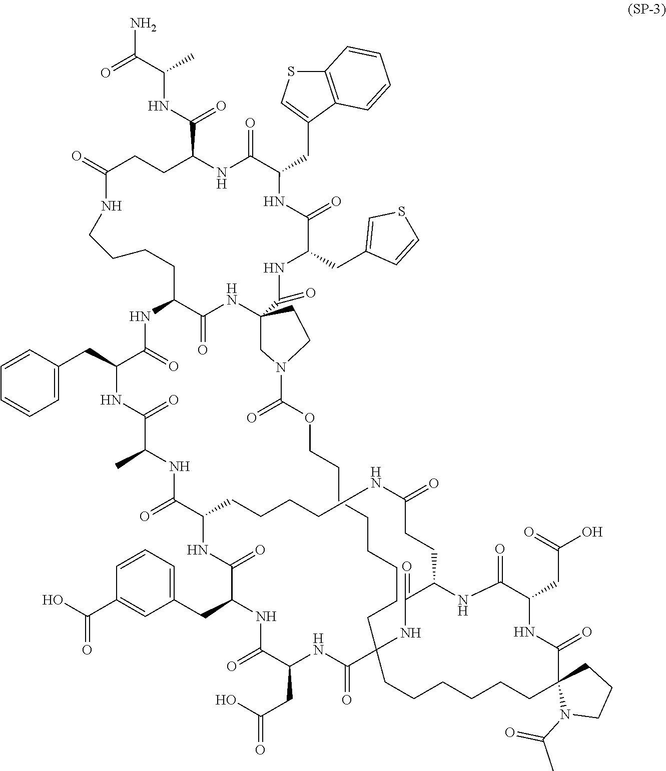 [SMILES: CC(=O)N1CCC[C@]12CCCCCCC13CCCCCCCCOC(=O)N4CC[C@@]5(C4)NC(=O)[C@H](CCCCNC(=O)CC[C@@H](C(=O)N[C@@H](C)C(N)=O)NC(=O)[C@H](Cc4csc6ccccc46)NC(=O)[C@H](Cc4ccsc4)NC5=O)NC(=O)[C@H](Cc4ccccc4)NC(=O)[C@H](C)NC(=O)[C@H](CCCCNC(=O)CC[C@H](NC(=O)[C@H](CC(=O)O)NC2=O)C(=O)N1)NC(=O)[C@H](Cc1cccc(C(=O)O)c1)NC(=O)[C@H](CC(=O)O)NC3=O]

or a salt thereof. In some embodiments, a compound has the structure of

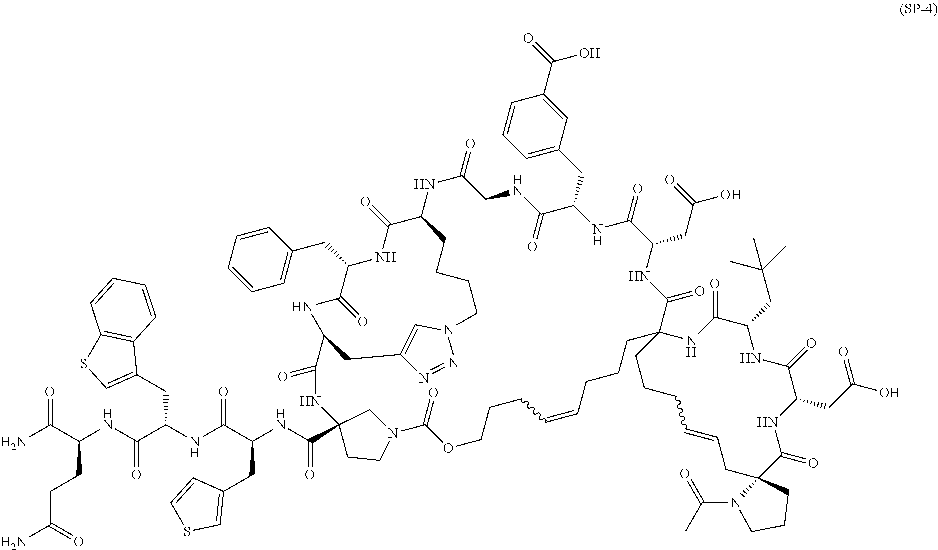

or a salt thereof. In some embodiments, a compound has the structure of

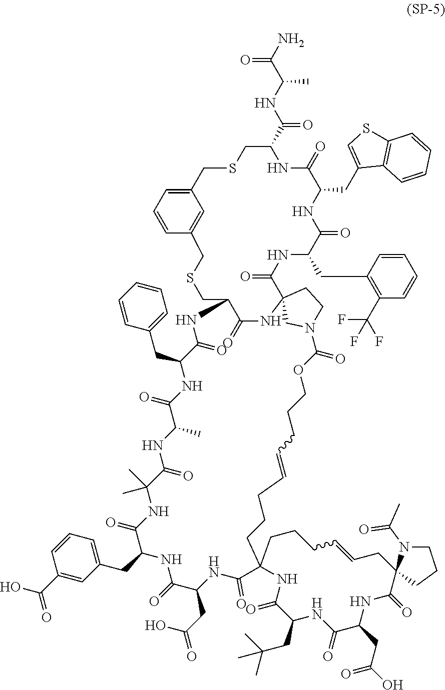 [SMILES: CC(=O)N1CCC[C@]12CC=CCCCC1(CCCC=CCCCOC(=O)N3CC[C@@]4(C3)NC(=O)[C@@H](CSCc3cccc(c3)CSC[C@H](C(=O)N[C@@H](C)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

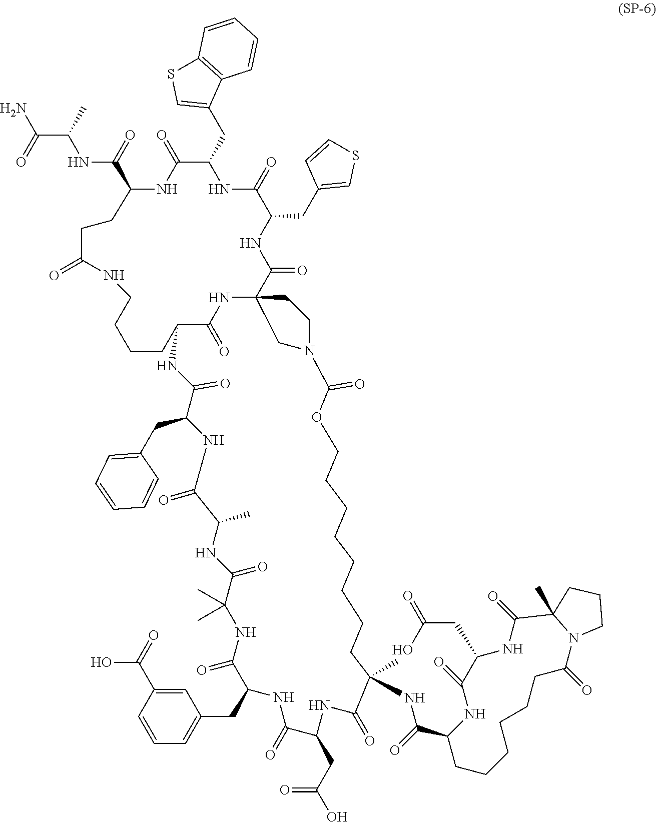 [SMILES: C[C@H](NC(=O)[C@@H]1CCC(=O)NCCCC[C@@H]2NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC(=O)[C@](C)(NC(=O)[C@@H]3CCCCCCC(=O)N4CCC[C@@]4(C)C(=O)N[C@@H](CC(=O)O)C(=O)N3)CCCCCCCCOC(=O)N3CC[C@@](C3)(NC2=O)C(=O)N[C@@H](Cc2ccsc2)C(=O)N[C@@H](Cc2csc3ccccc23)C(=O)N1)C(N)=O]

or a salt thereof. In some embodiments, a compound has the structure of

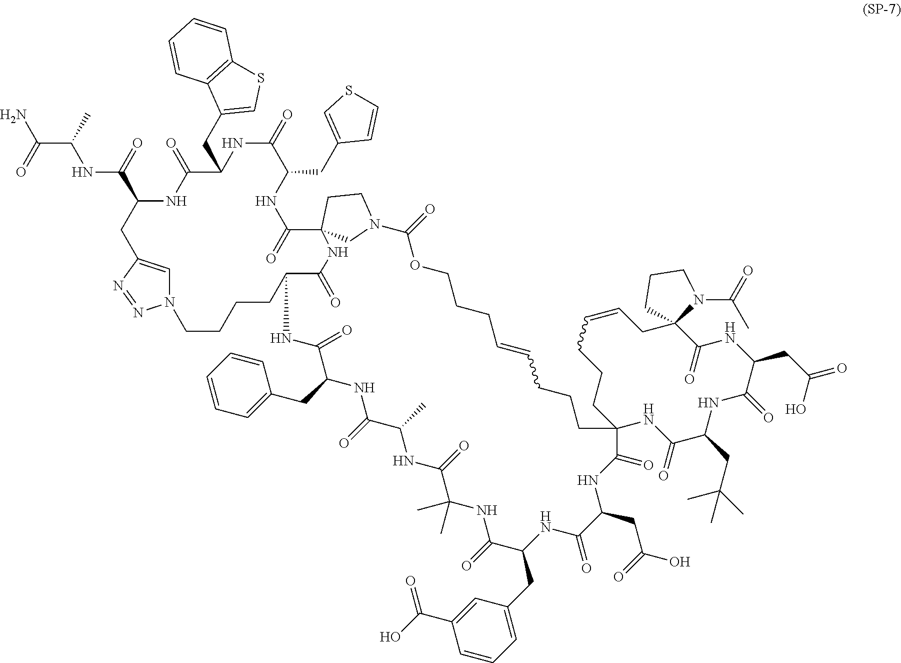 [SMILES: CC(=O)N1CCC[C@]12CC=CCCCC1(CCCC=CCCCOC(=O)N3CC[C@@]4(C3)NC(=O)[C@H](CCCCn3cc(nn3)C[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

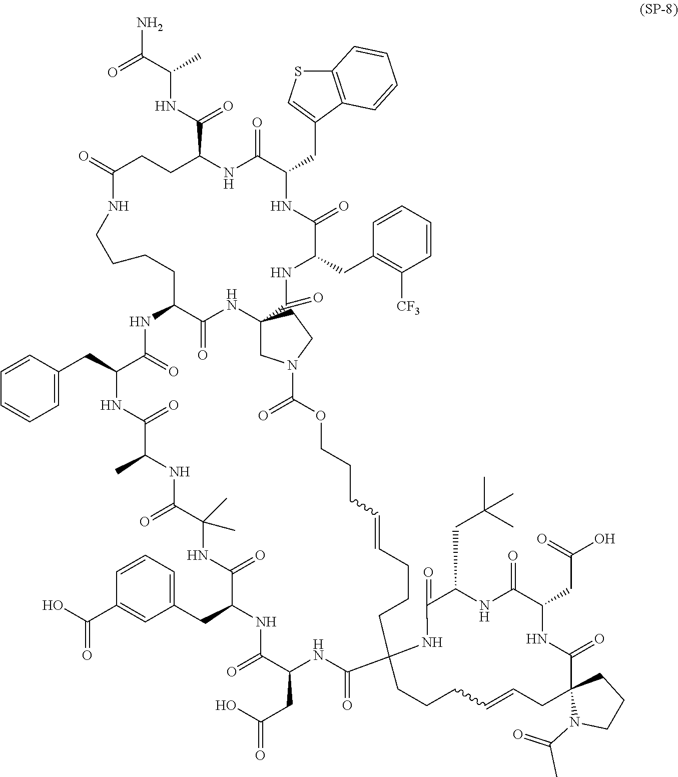 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

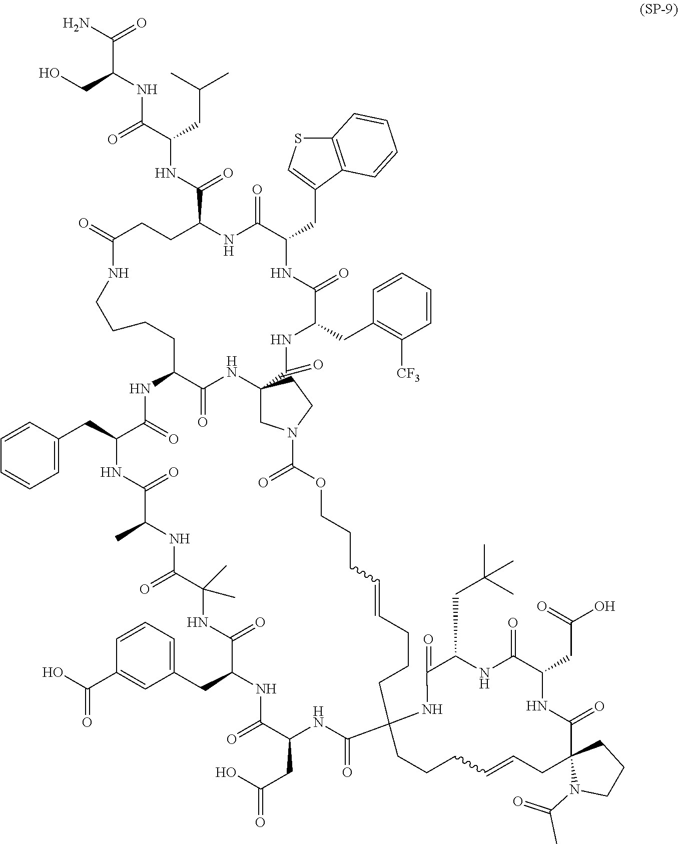 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](CC(C)C)C(=O)N[C@@H](CO)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

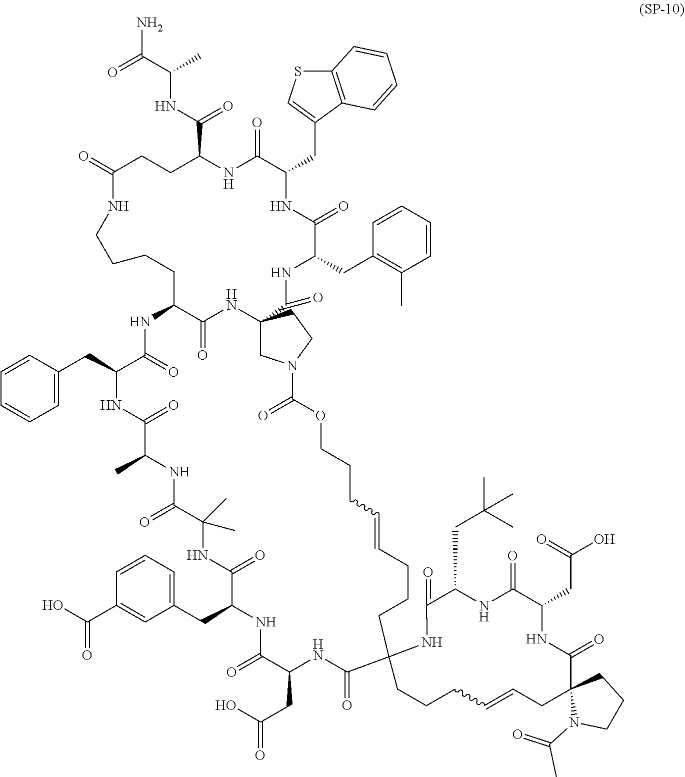 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3C)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

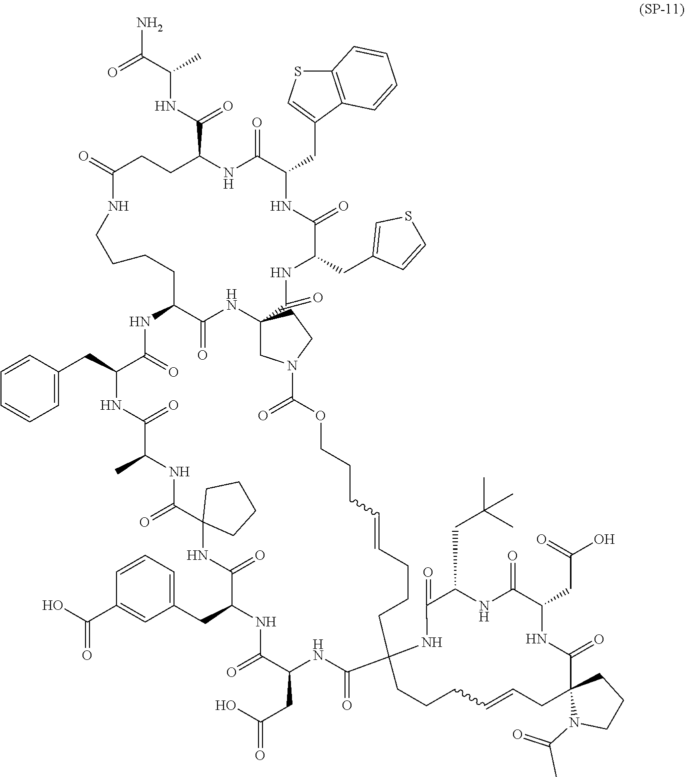 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C3(CCCC3)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

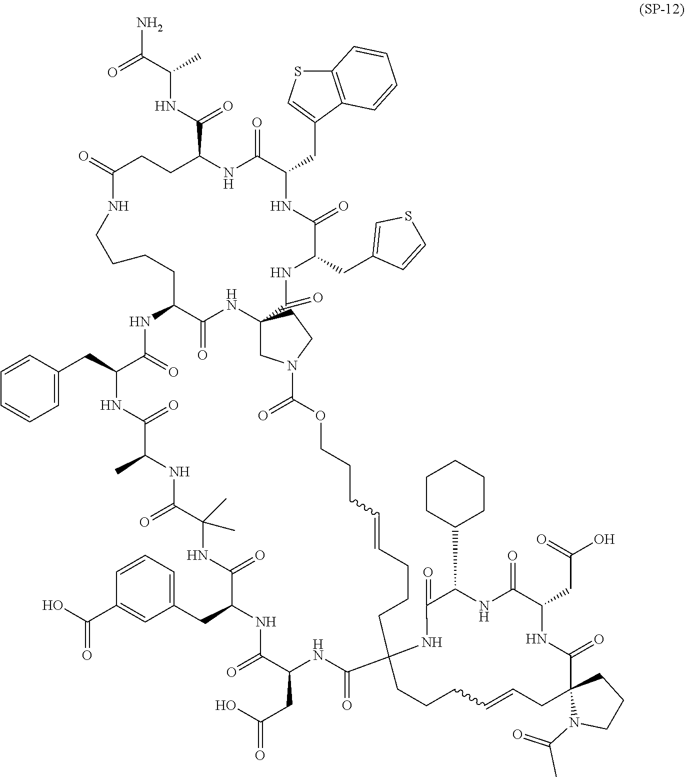 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](C1CCCCC1)NC(=O)[C@H](CC(=O)O)NC2=O]

or a salt thereof. In some embodiments, a compound has the structure of

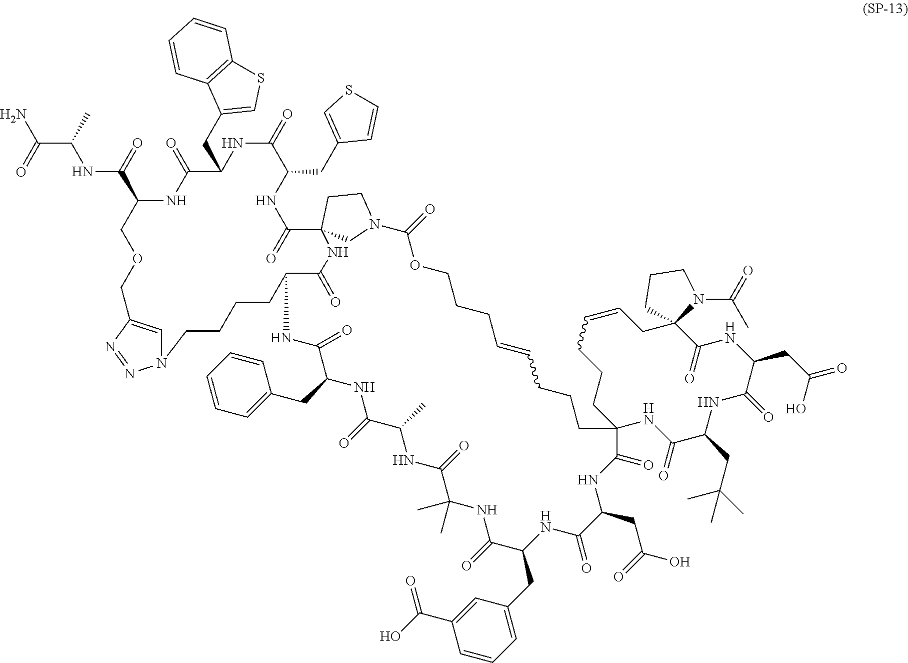 [SMILES: CC(=O)N1CCC[C@]12CC=CCCCC1(CCCC=CCCCOC(=O)N3CC[C@@]4(C3)NC(=O)[C@H](CCCCn3cc(nn3)COC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

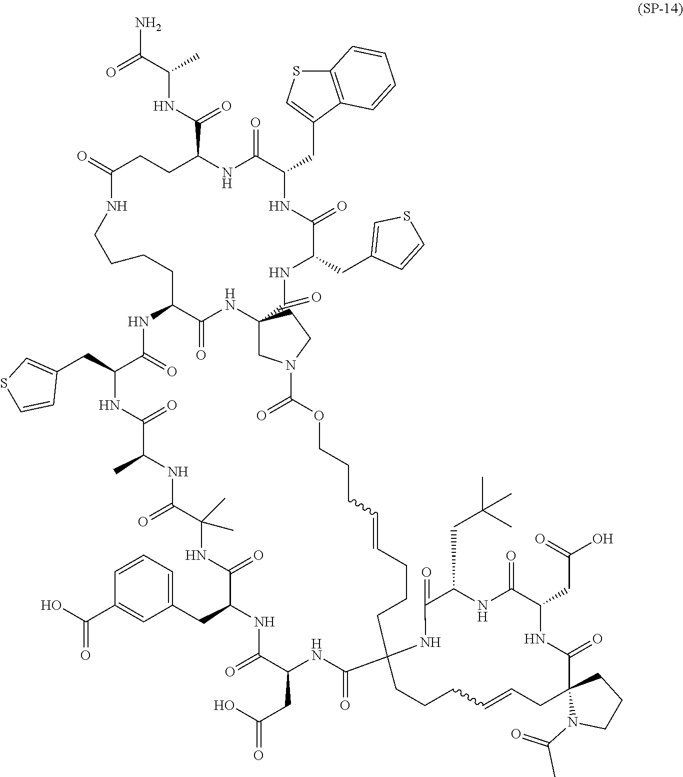 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccsc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a compound has the structure of

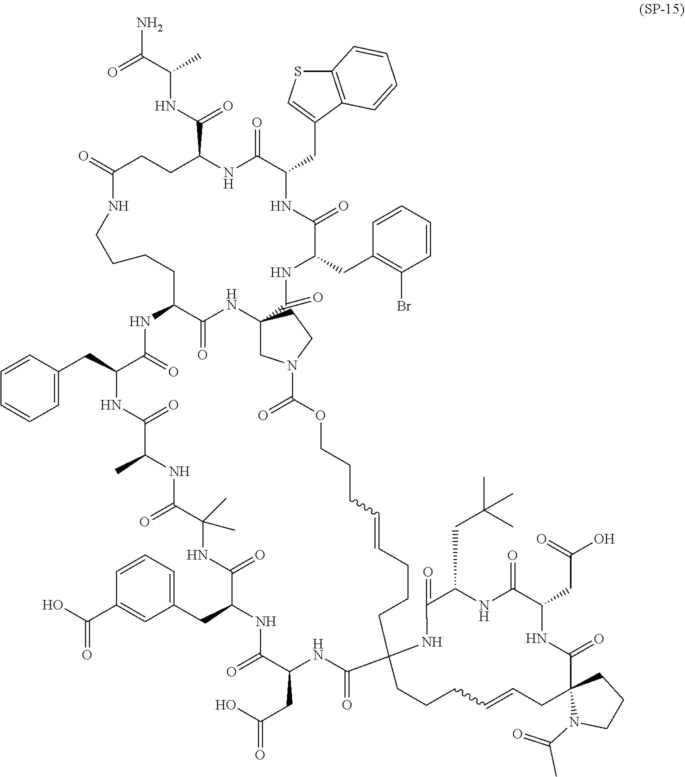 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3Br)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a double bond of a (i, i+2) staple is E. In some embodiments, a double bond of a (i, i+2) staple is Z In some embodiments, a double bond of a (i, i+3) staple is E. In some embodiments, a double bond of a (i, i+3) staple is Z In some embodiments, a double bond of a (i, i+7) staple is E. In some embodiments, a double bond of a (i, i+7) staple is Z In some embodiments, both double bonds are E. In some embodiments, both double bonds are Z. In some embodiments, a (i, i+3) staple is E, and the other is Z. In some embodiments, a (i, i+3) staple is Z, and the other is E. In some embodiments, a (i, i+4) staple is E, and the other is Z. In some embodiments, a (i, i+4) staple is Z, and the other is E. In some embodiments, a double bond of a (i, i+7) staple is Z, and a double bond of a second staple (e.g., (i, i+2), (i, i+3), (i, i+4), etc.) is E. In some embodiments, a double bond of a (i, i+7) staple is Z, and a double bond of a second staple (e.g., (i, i+2), (i, i+3), (i, i+4), etc.) is Z. In some embodiments, a double bond of a (i, i+7) staple is E, and a double bond of a second staple (e.g., (i, i+2), (i, i+3), (i, i+4), etc.) is E. In some embodiments, a double bond of a (i, i+7) staple is E, and a double bond of a second staple (e.g., (i, i+2), (i, i+3), (i, i+4), etc.) is Z. In some embodiments, two staples are bonded to a chiral center (e.g., a carbon atom in B5), and the chiral center is R. In some embodiments, two staples are bonded to a chiral center (e.g., a carbon atom in B5), and the chiral center is S.

In some embodiments, a compound has the structure selected from below or a salt thereof:

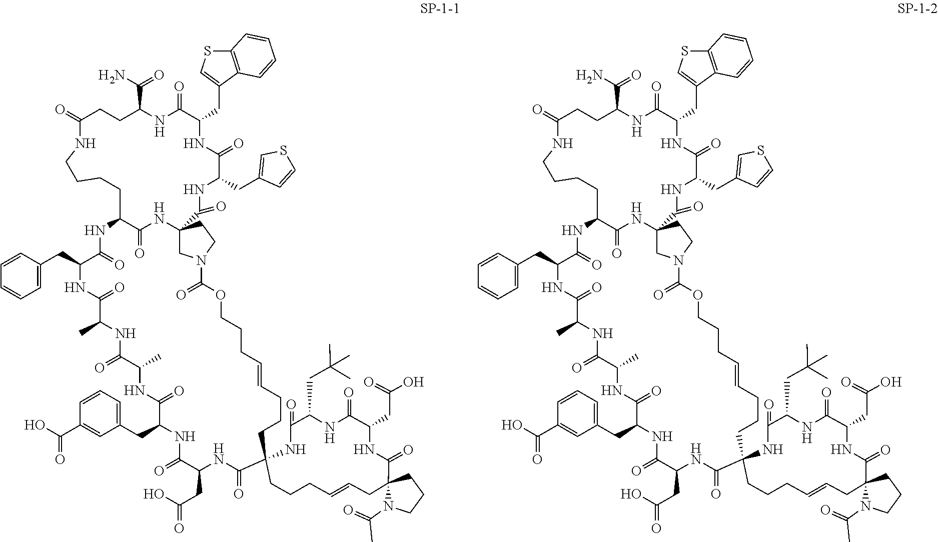

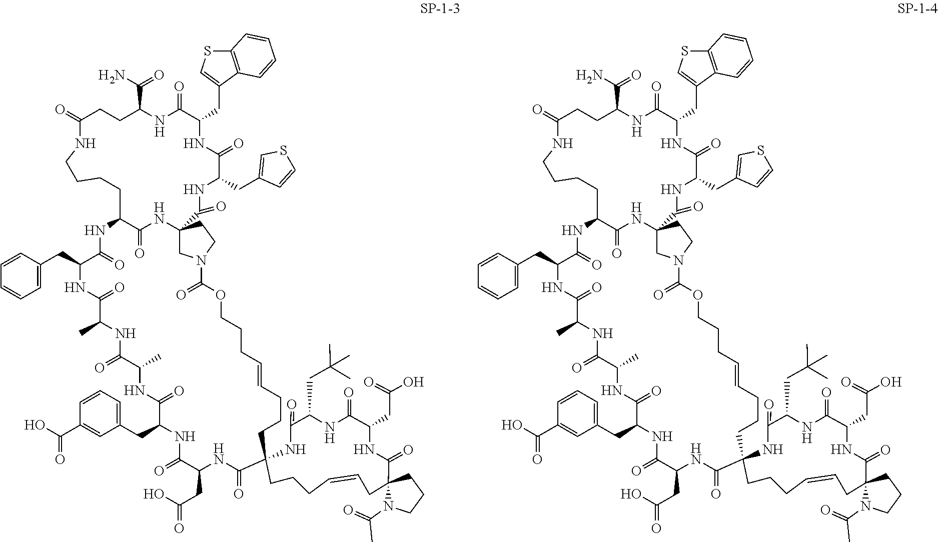

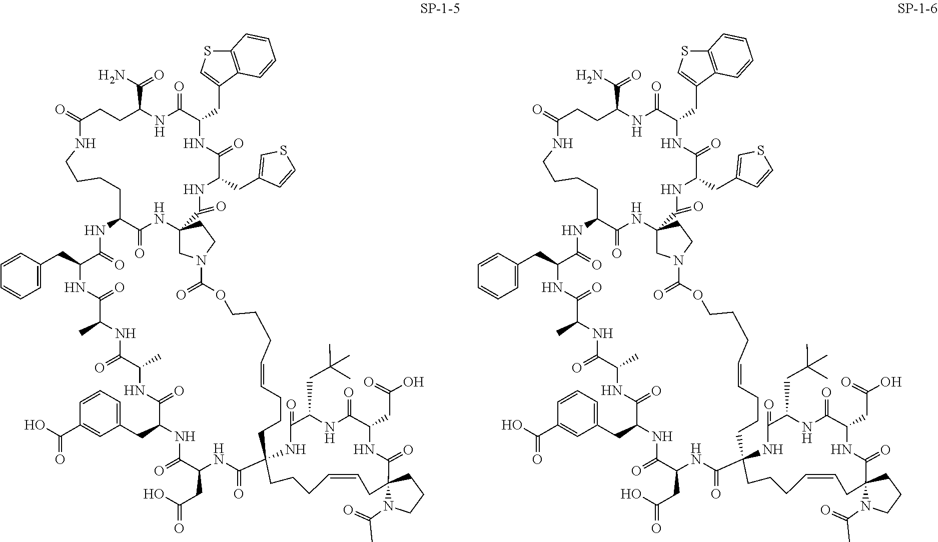

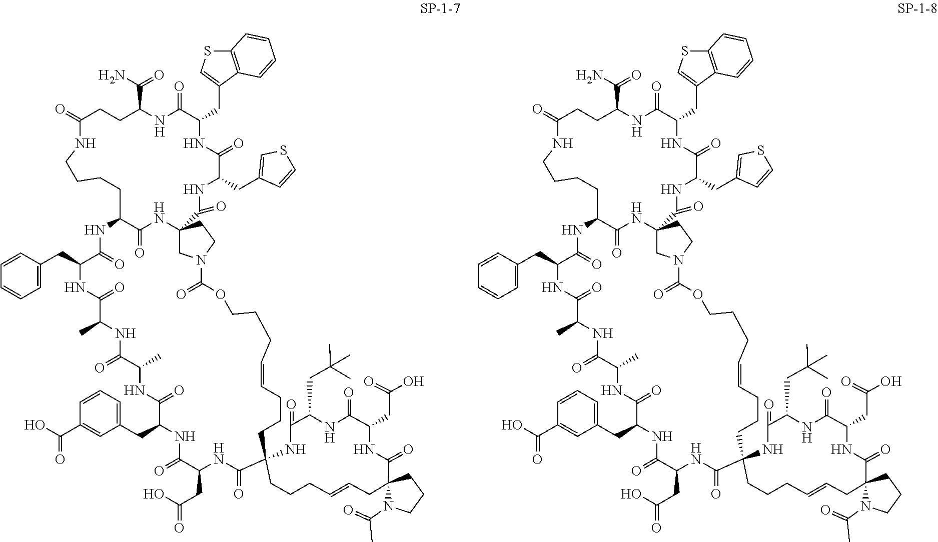

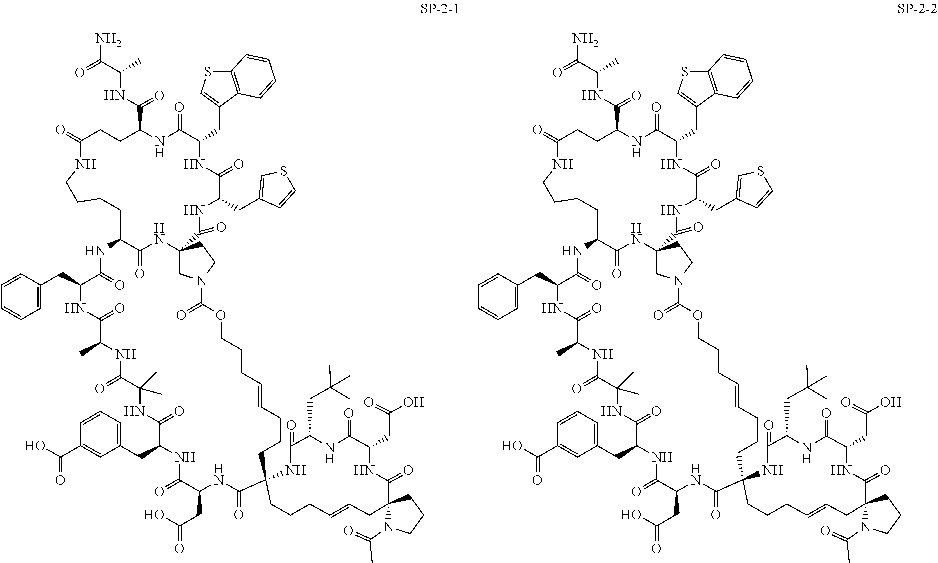

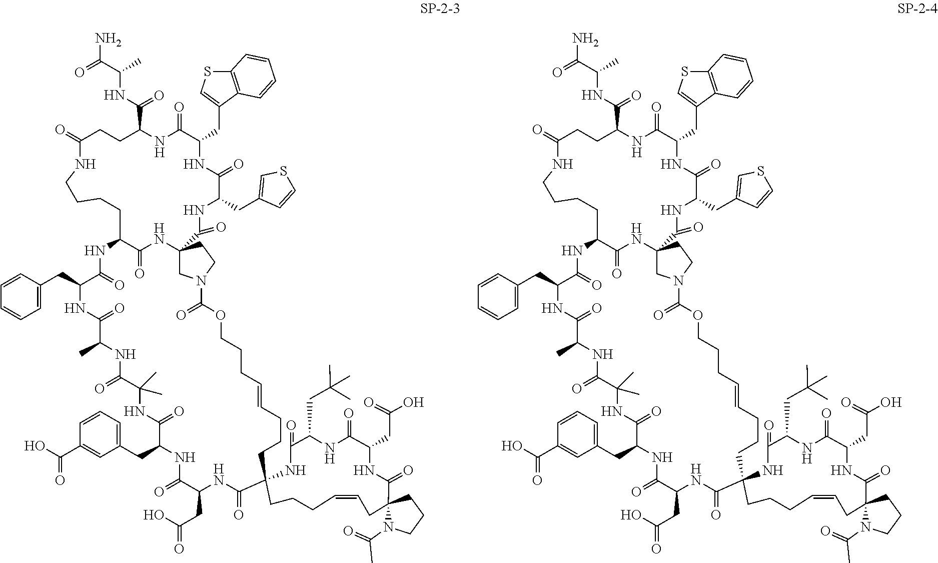

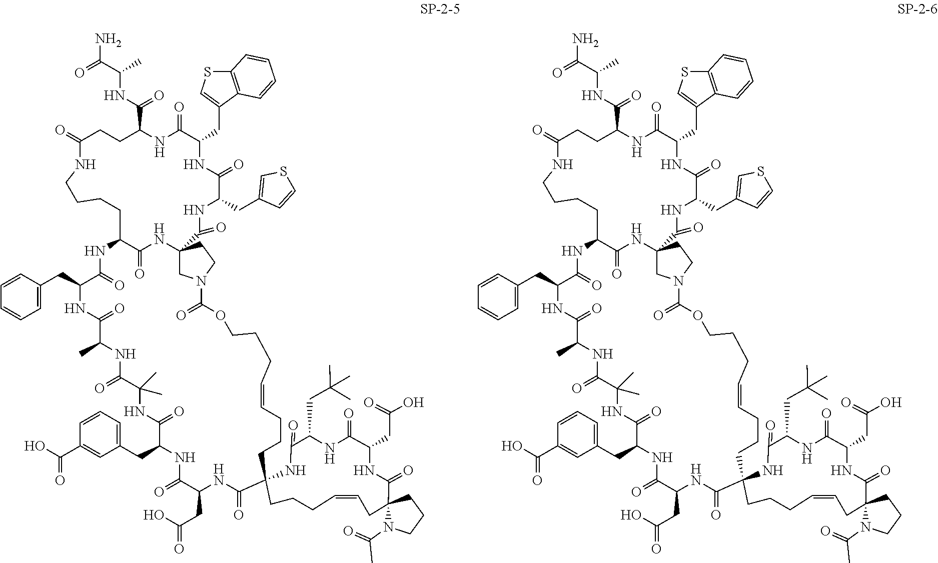

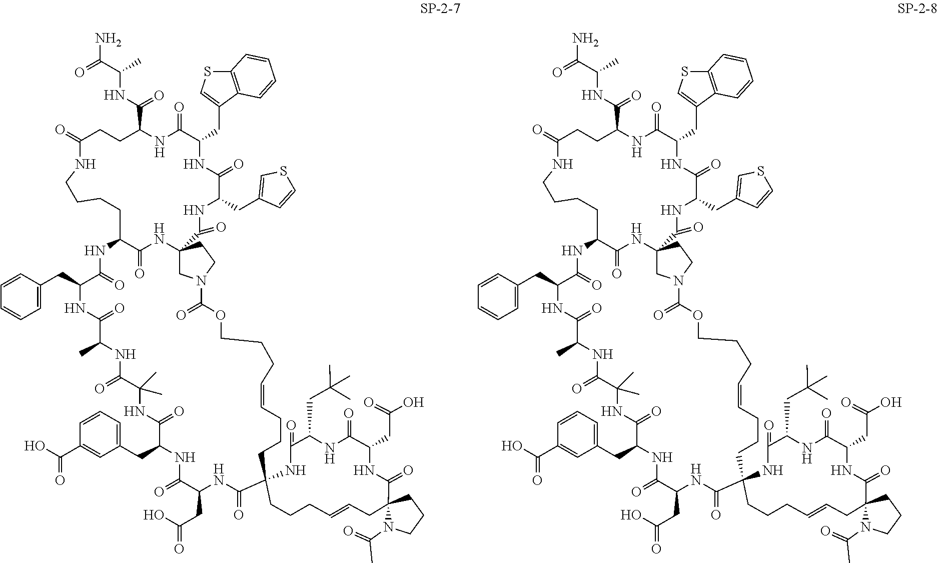

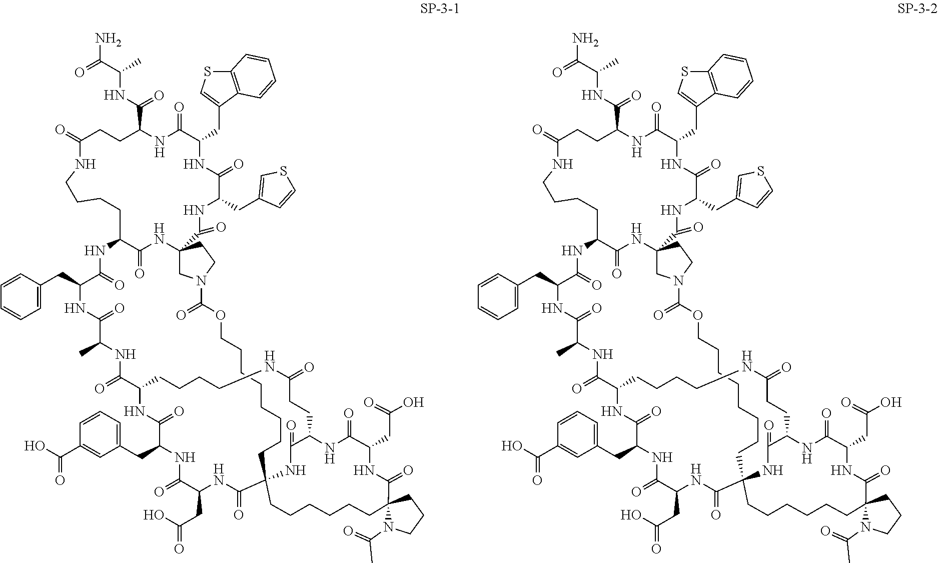

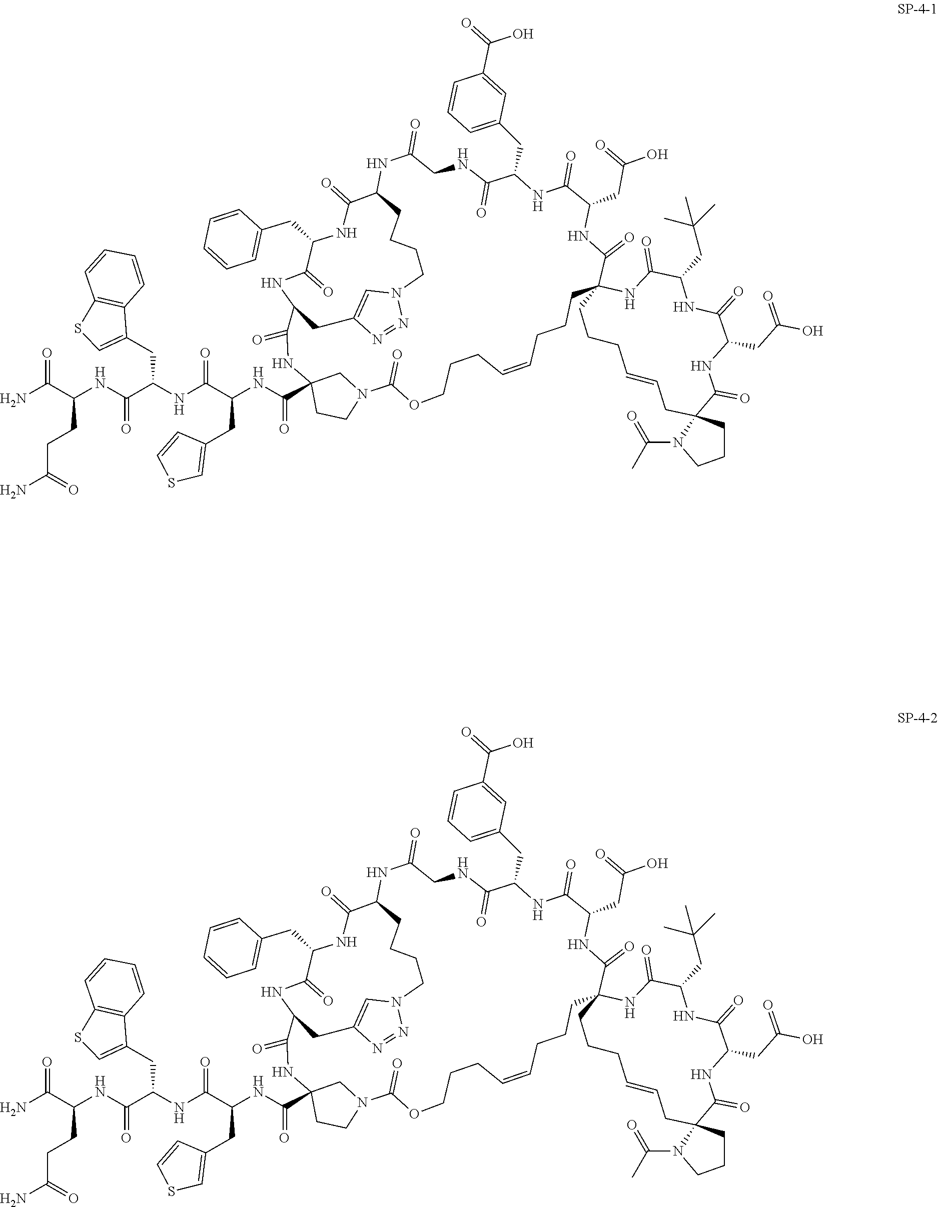

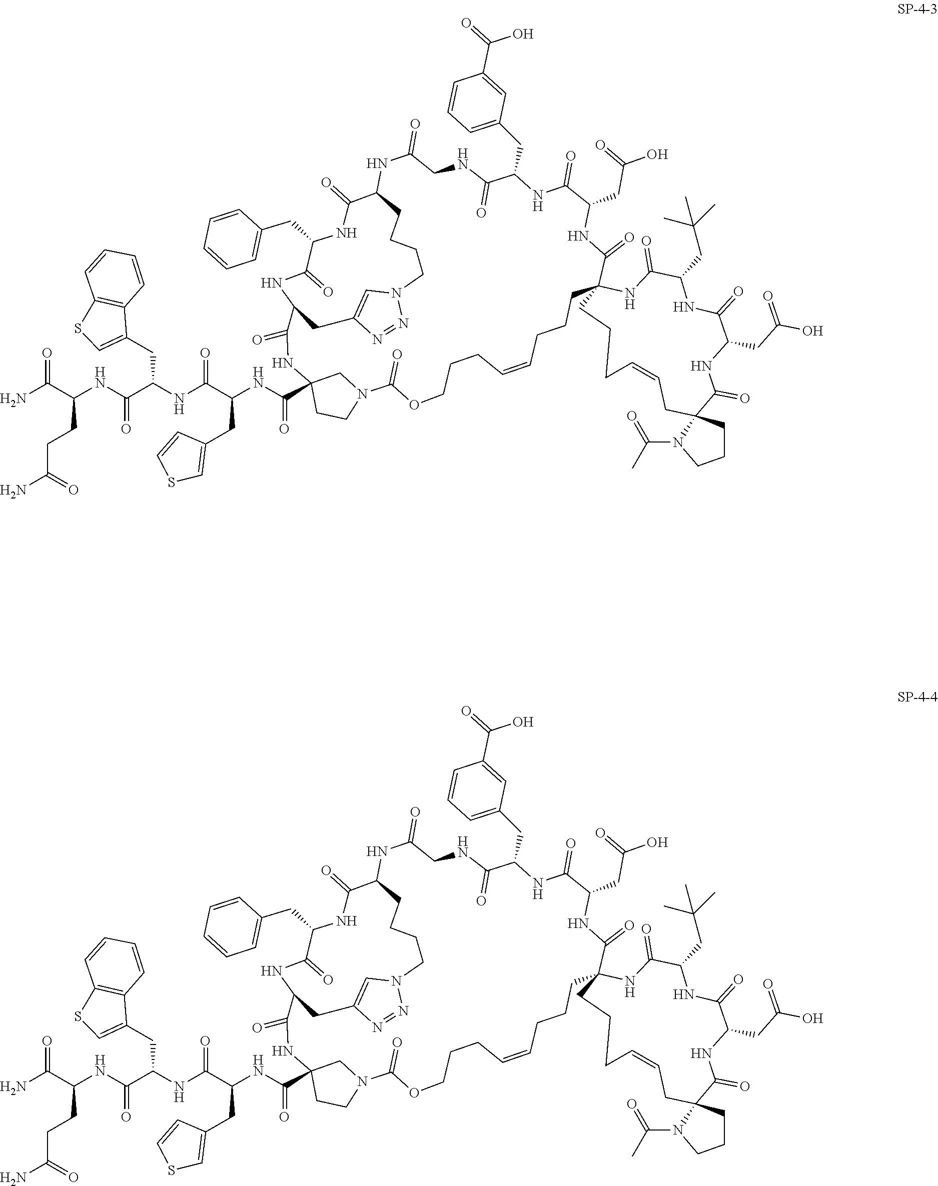

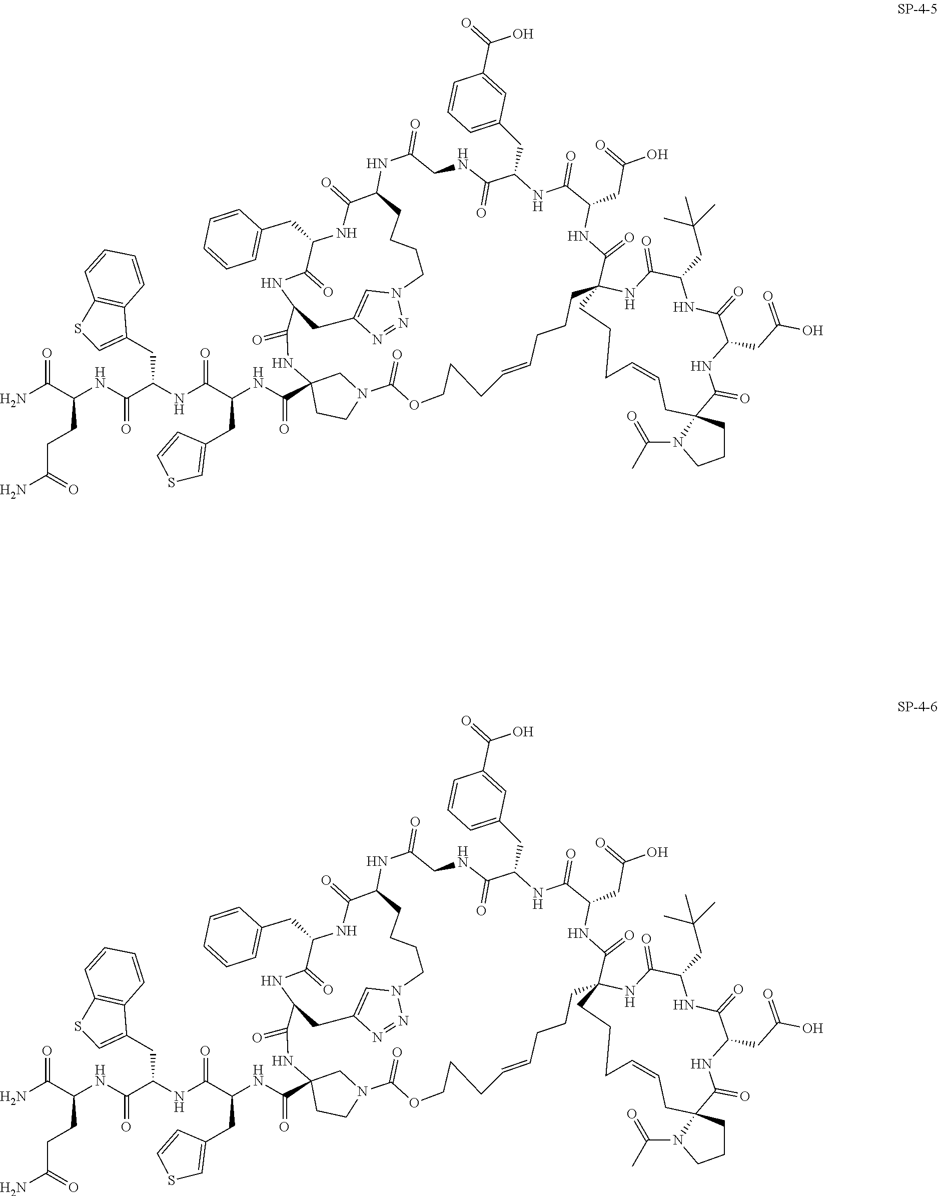

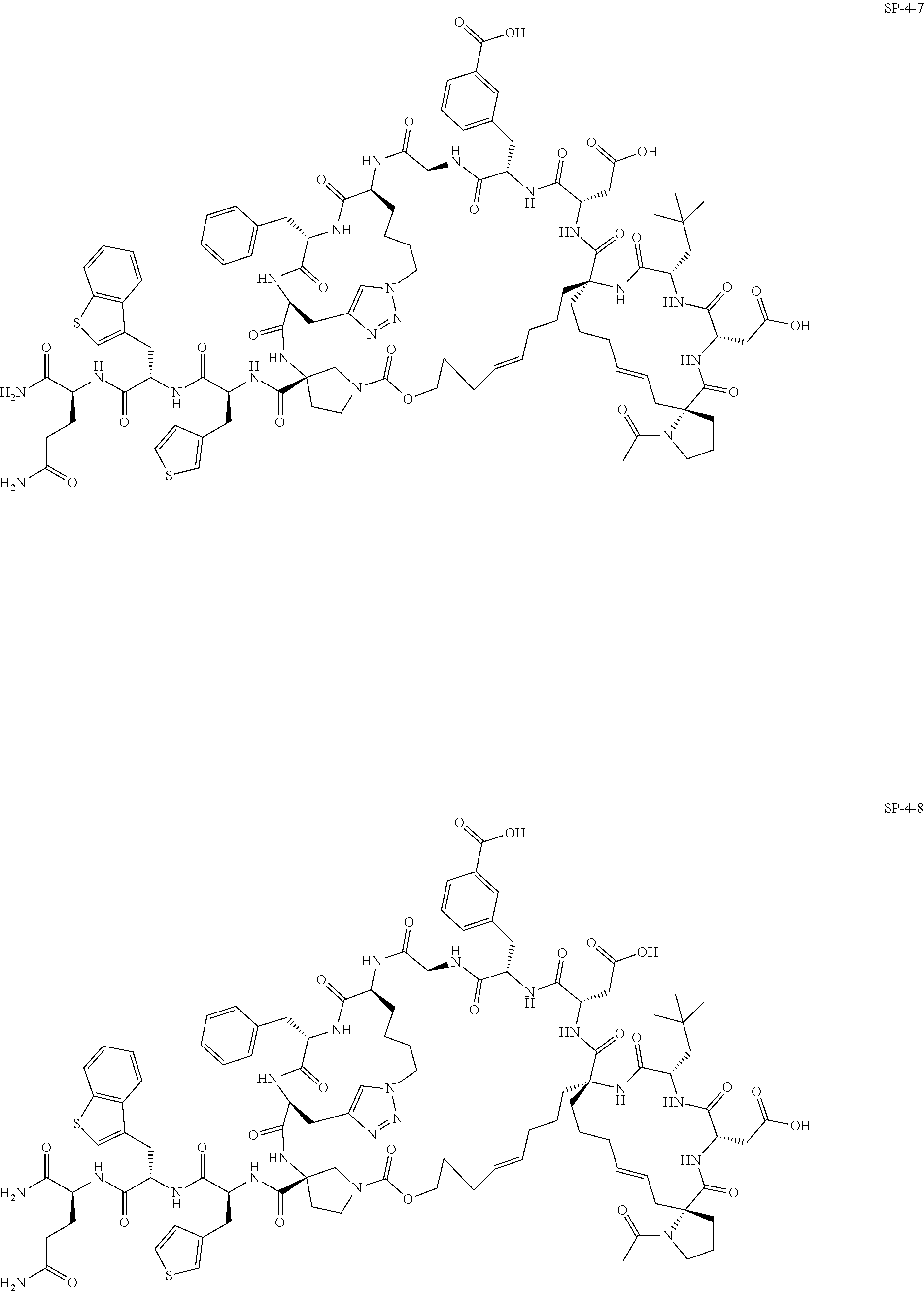

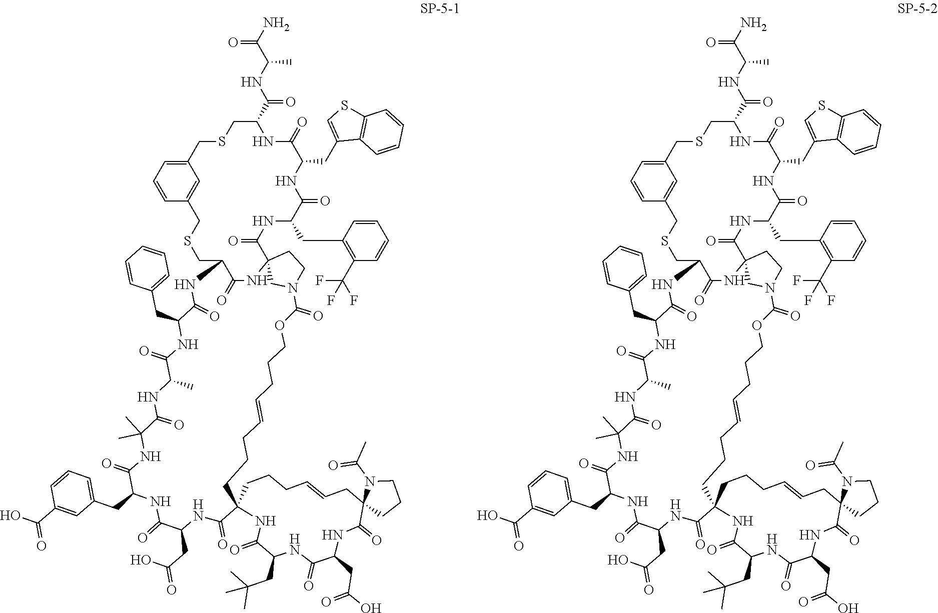

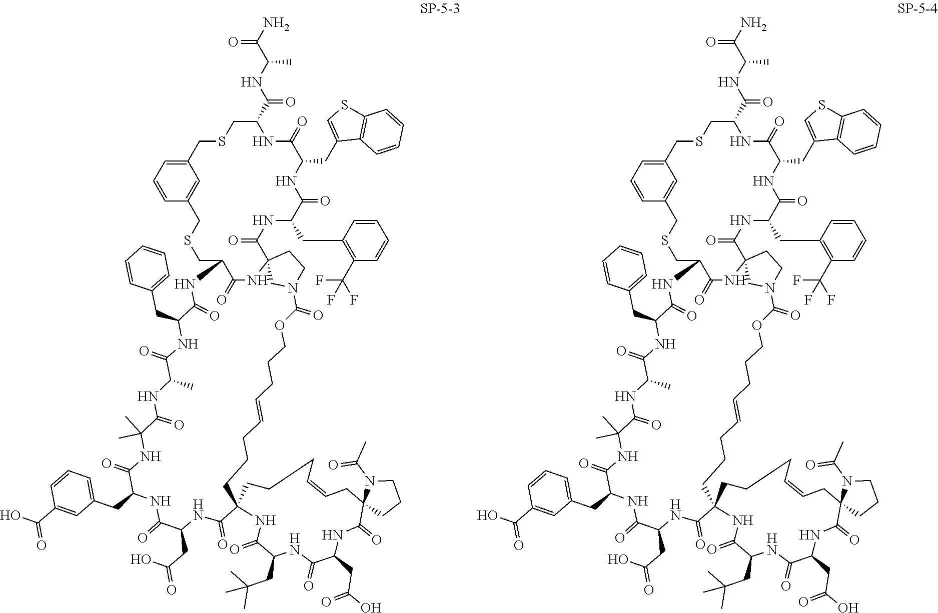

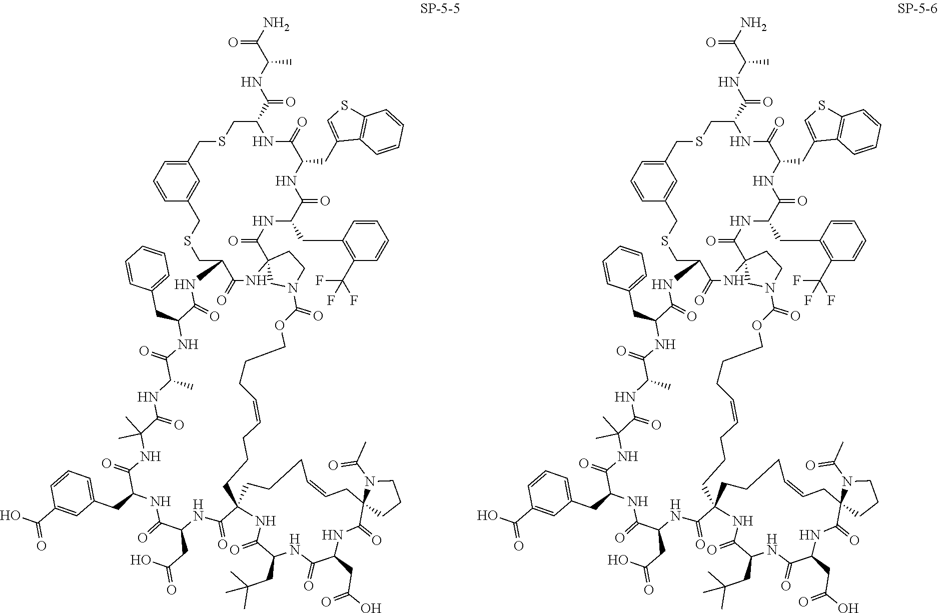

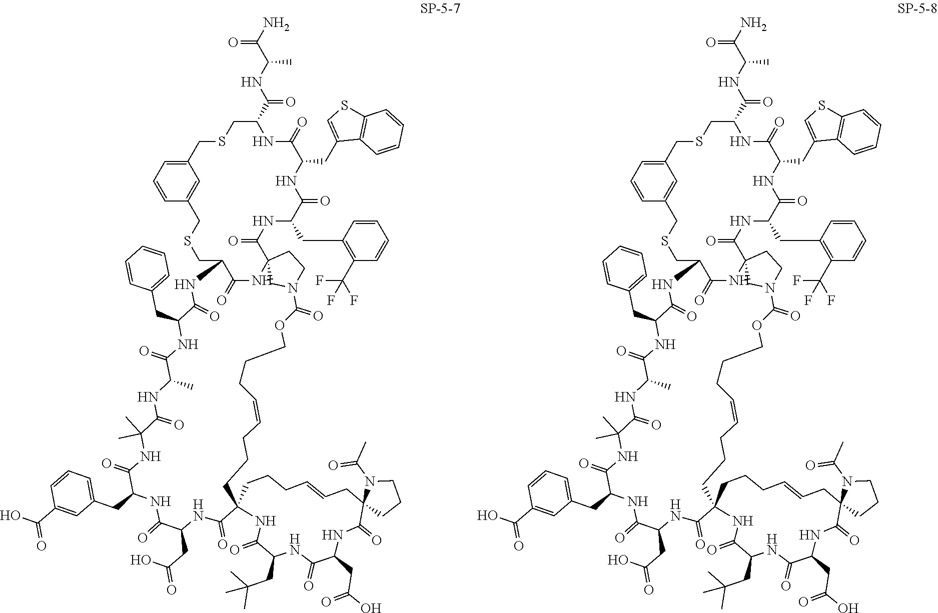

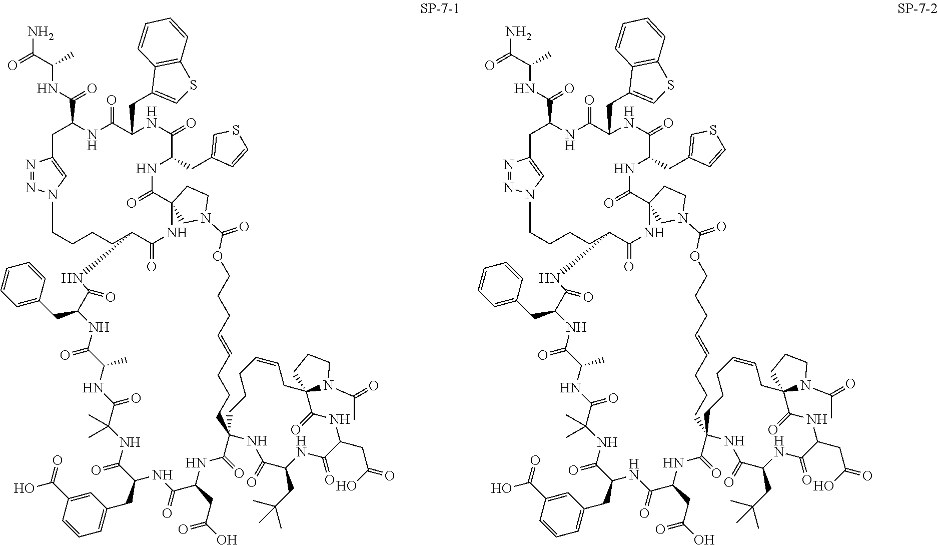

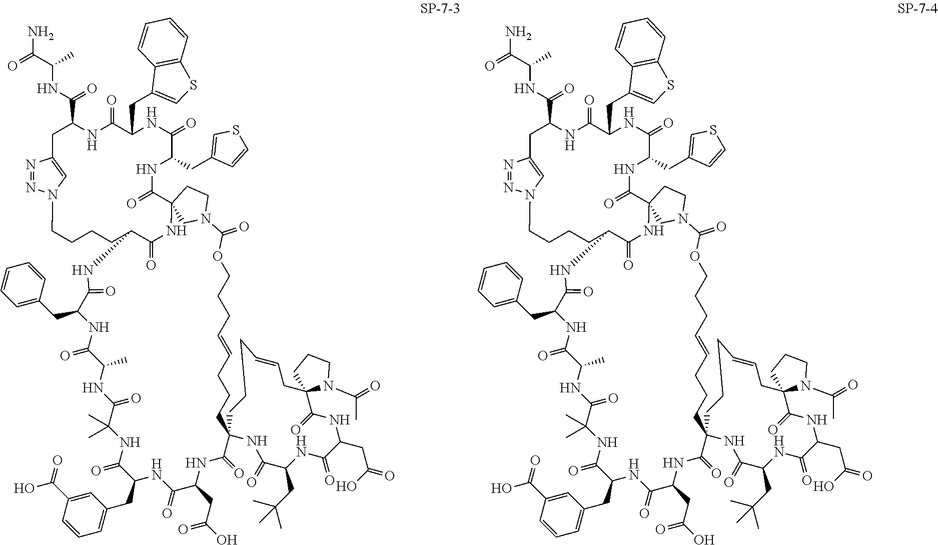

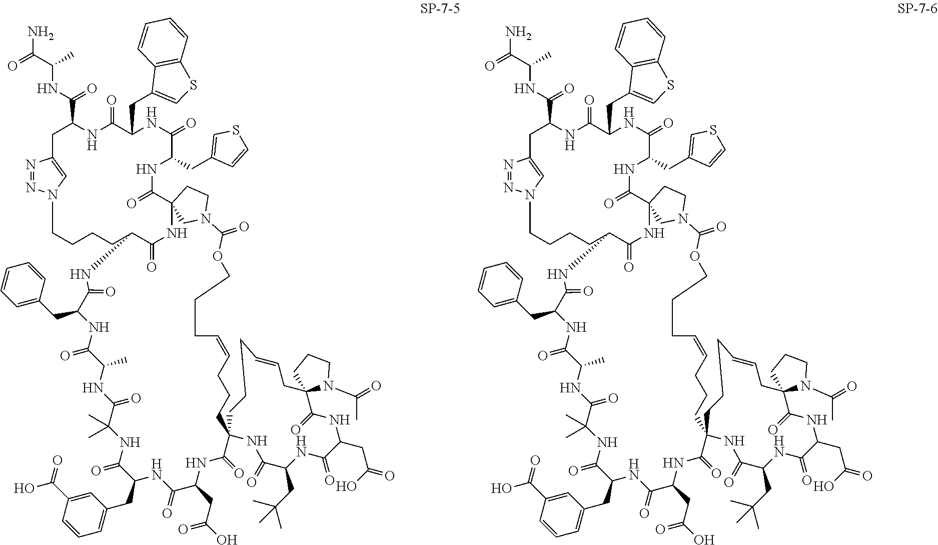

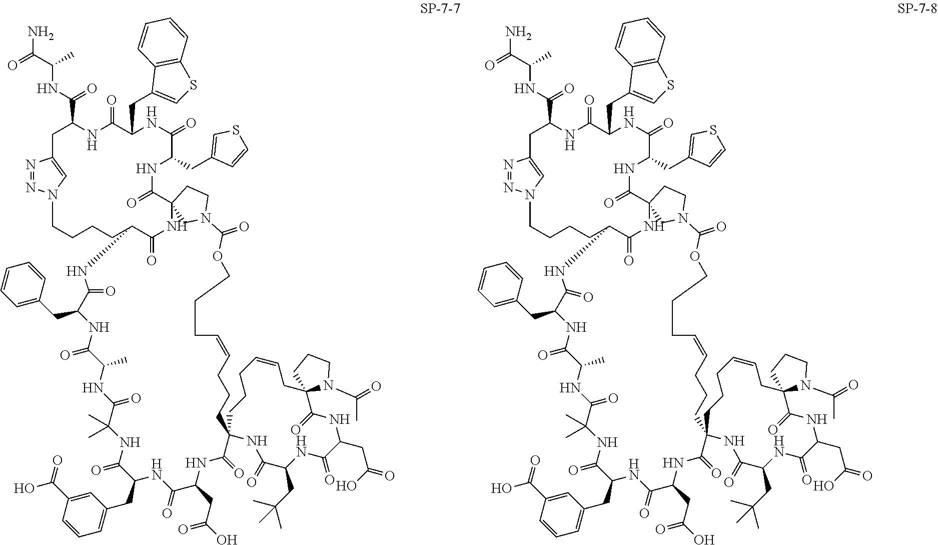

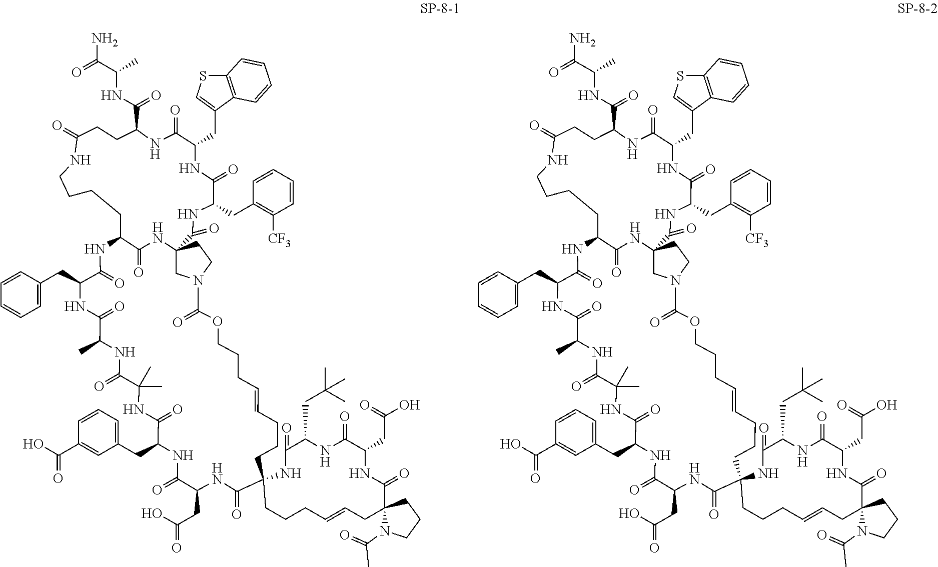

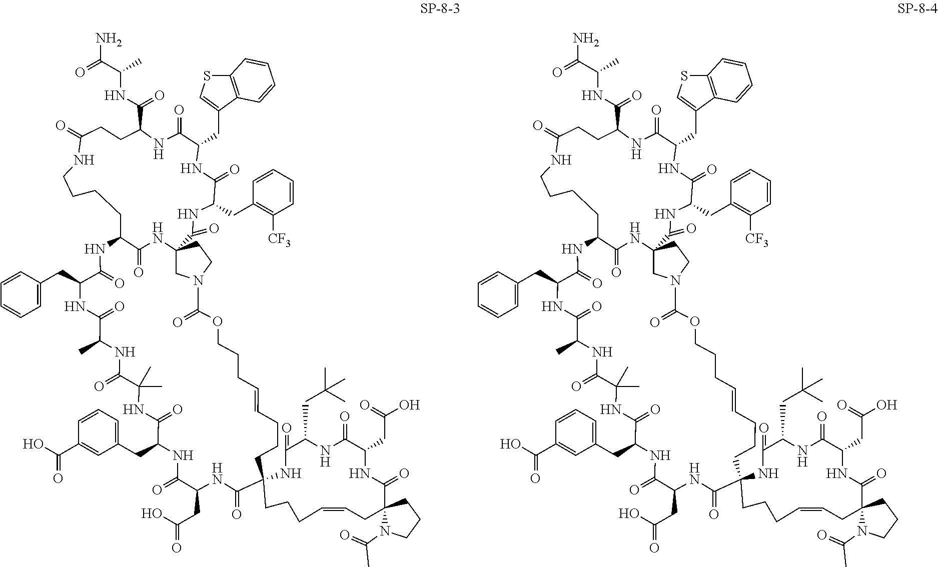

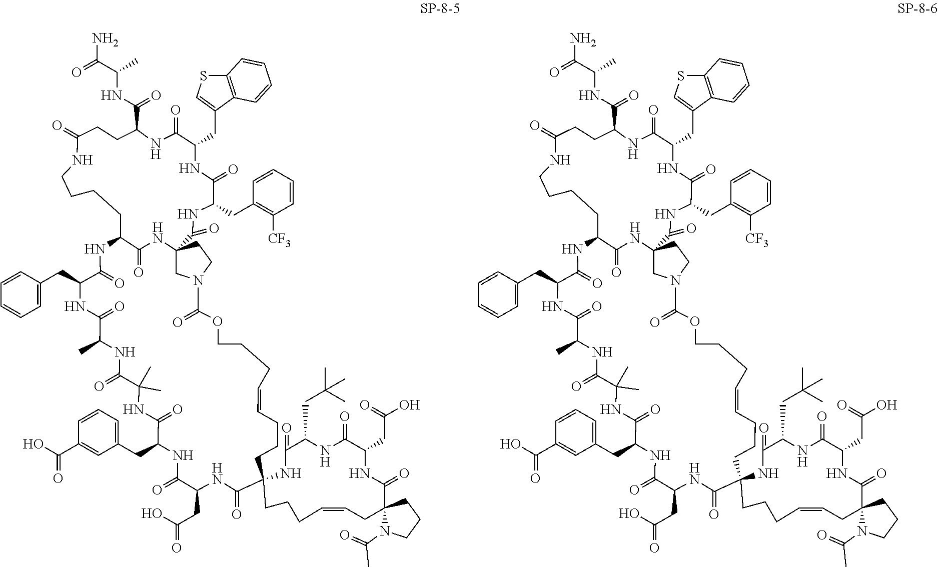

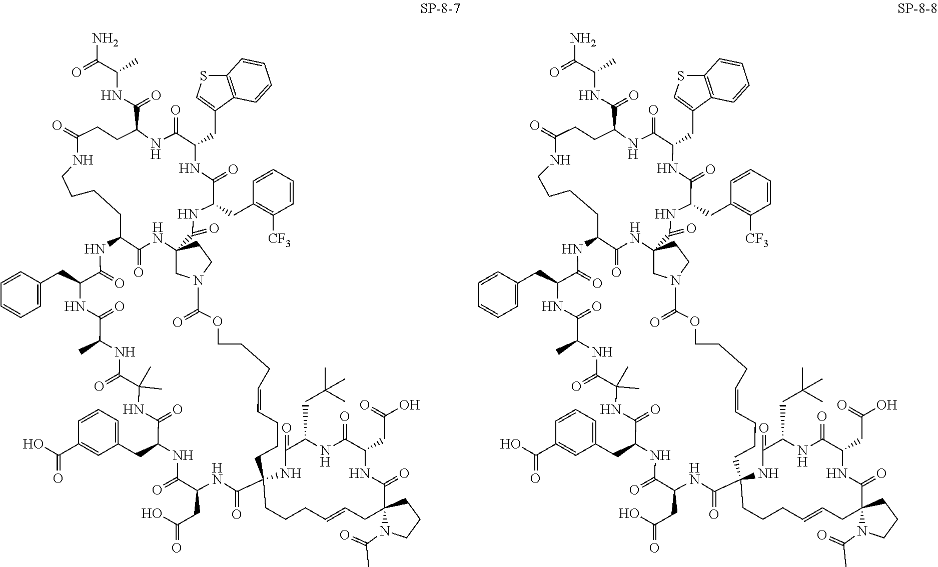

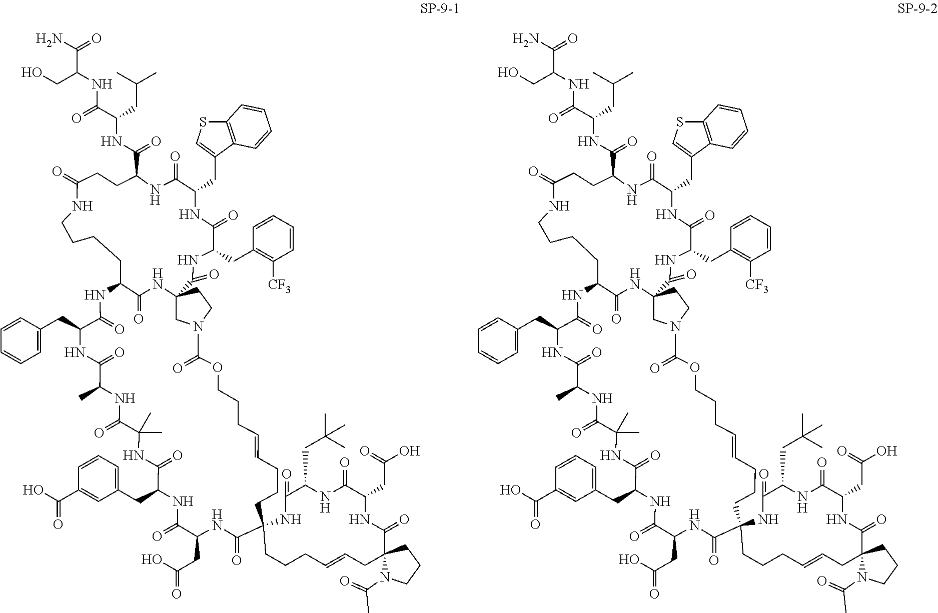

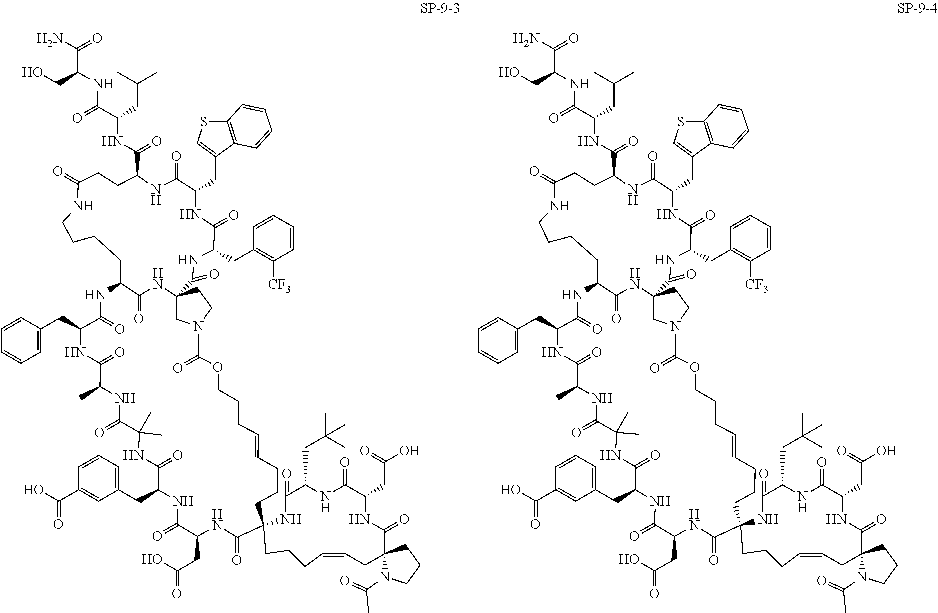

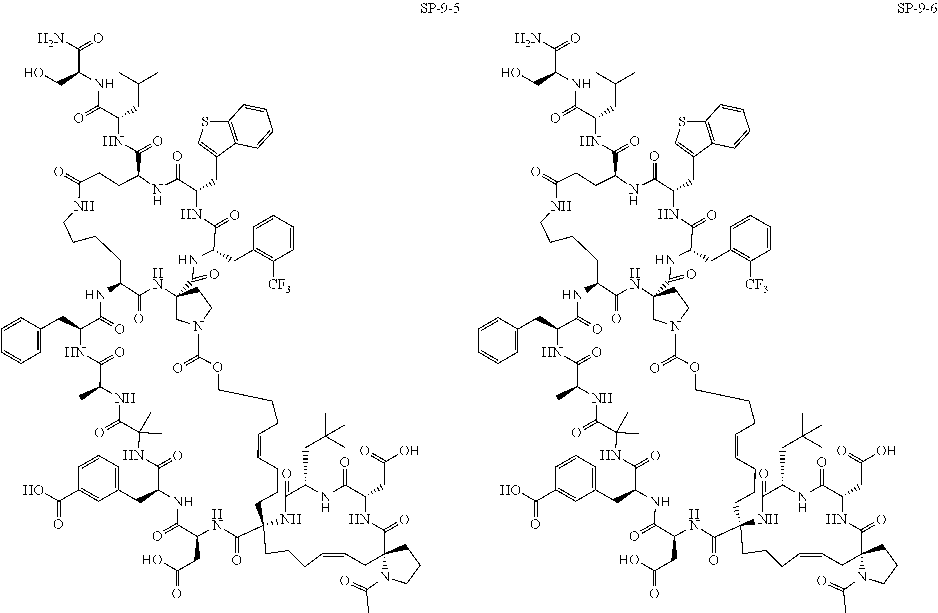

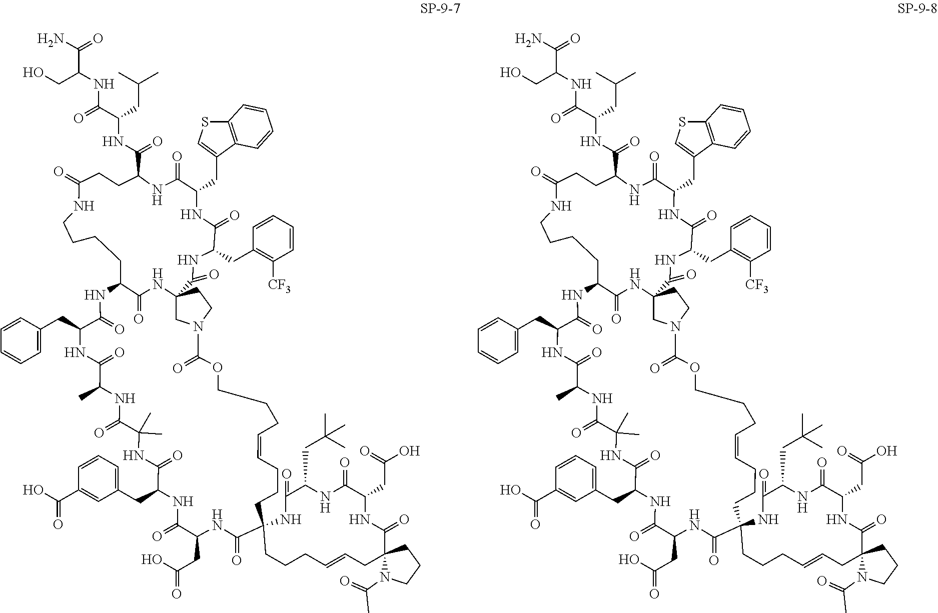

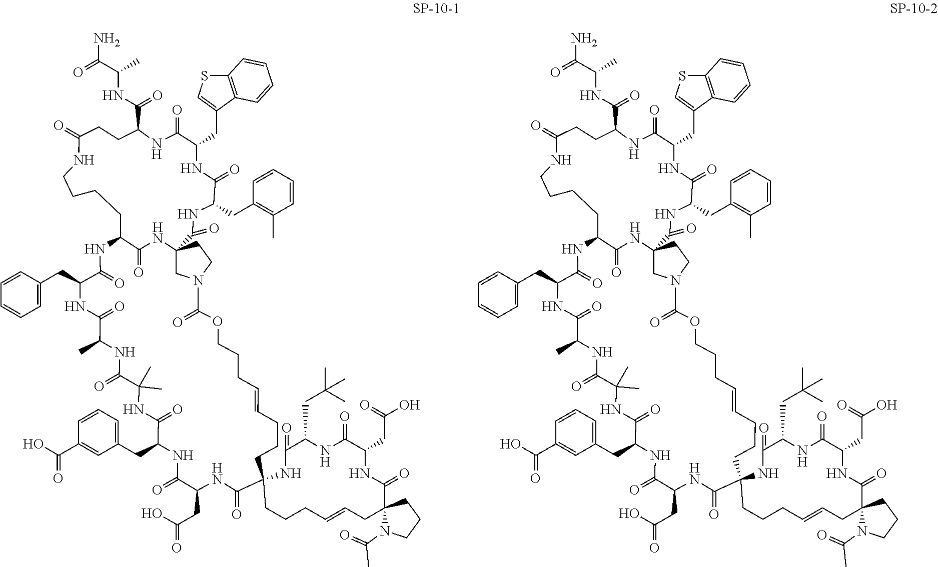

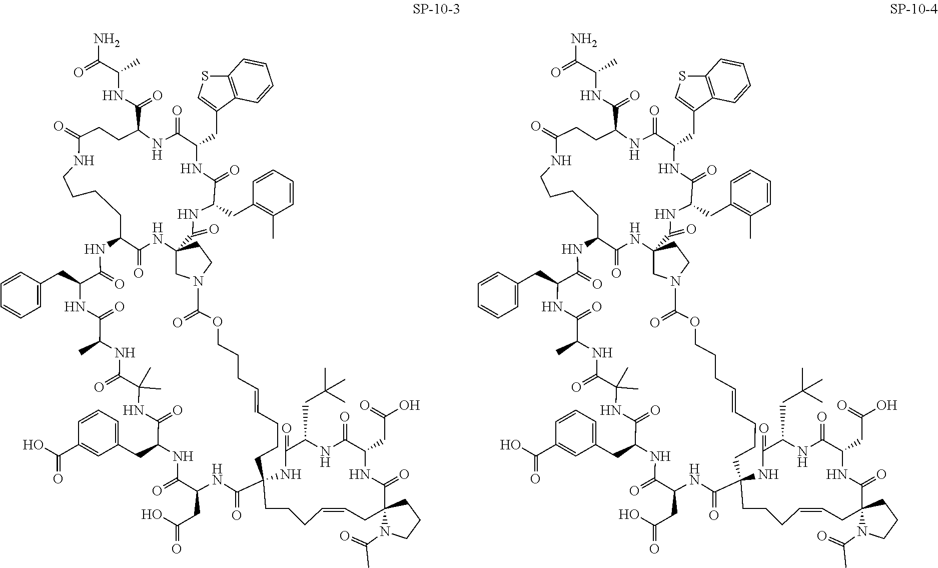

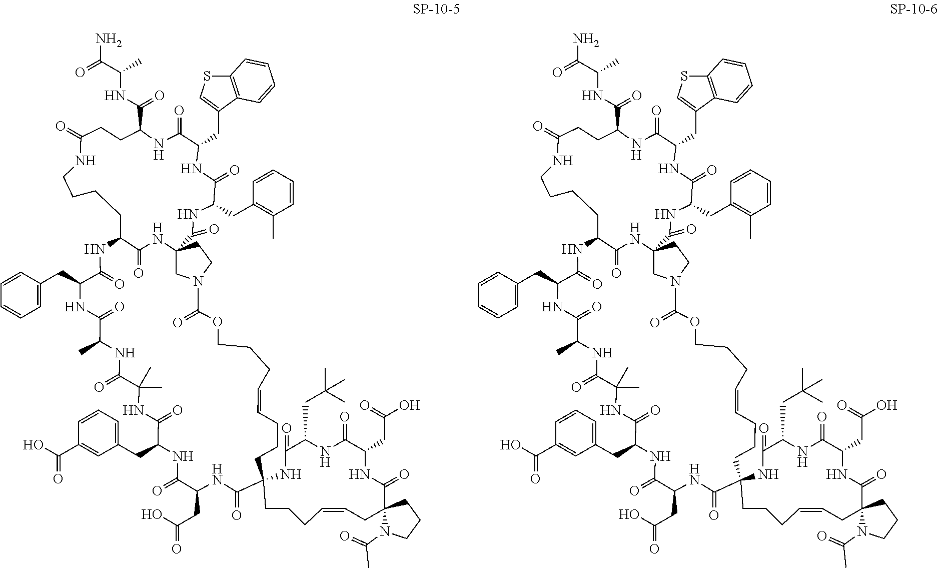

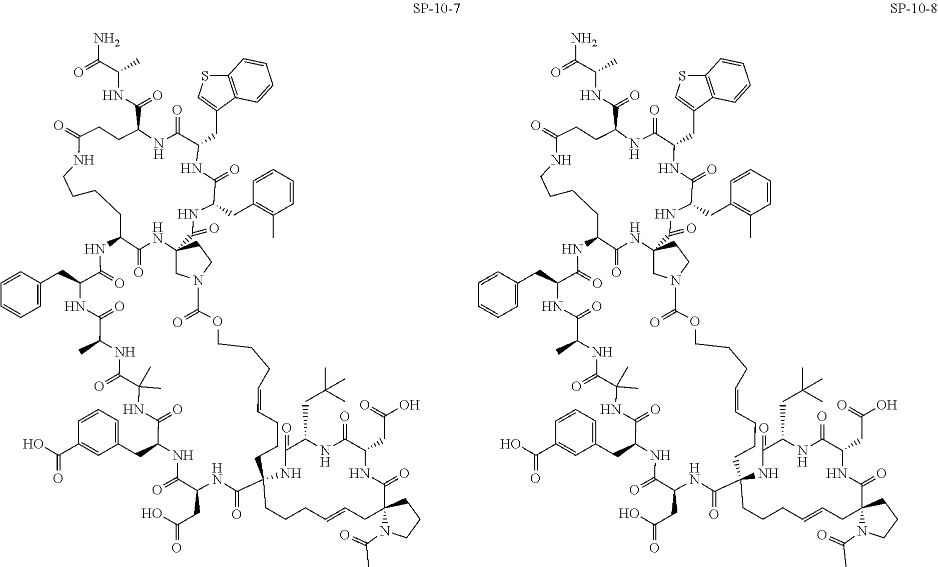

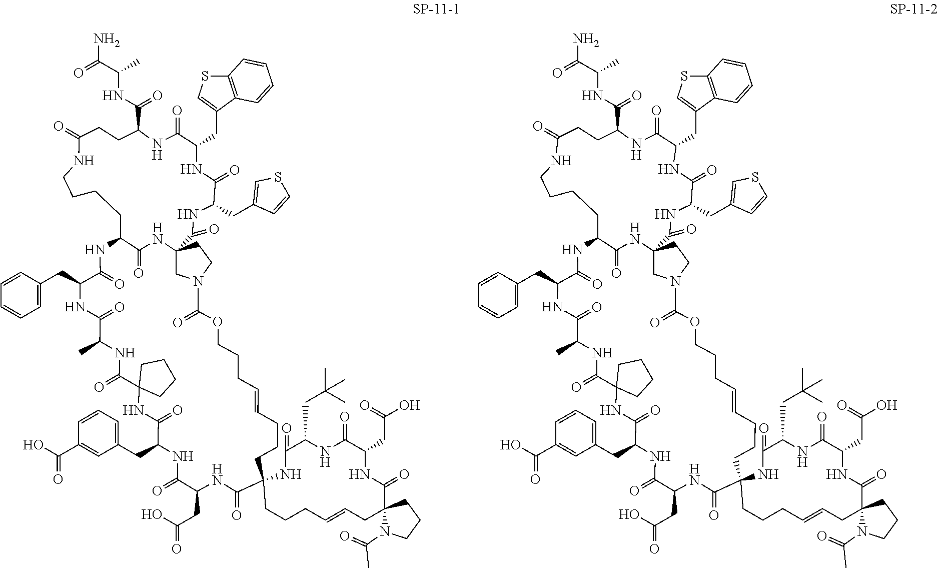

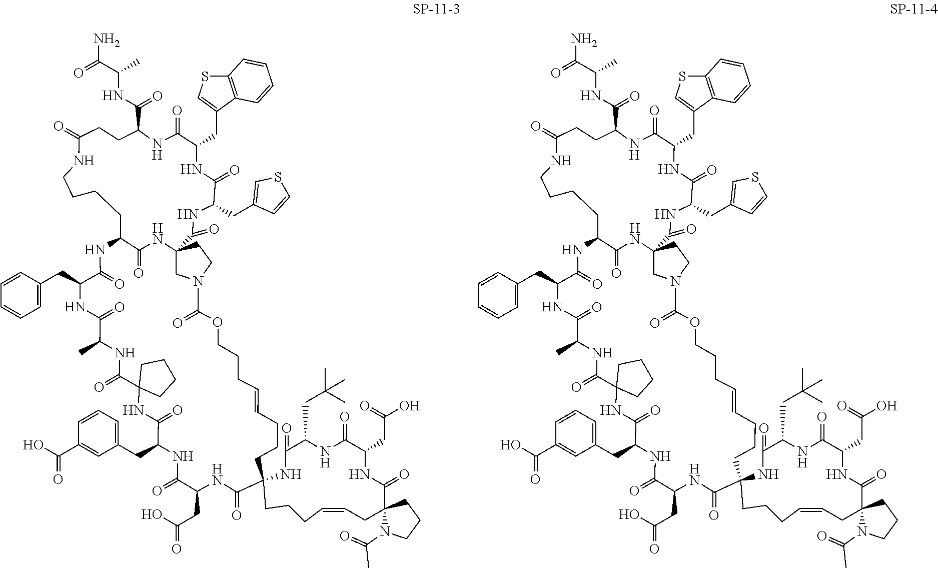

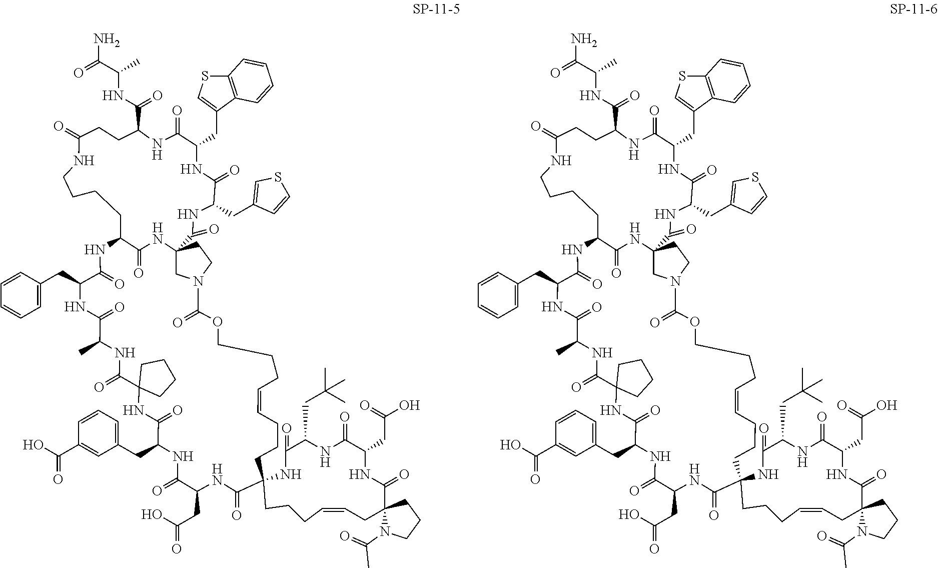

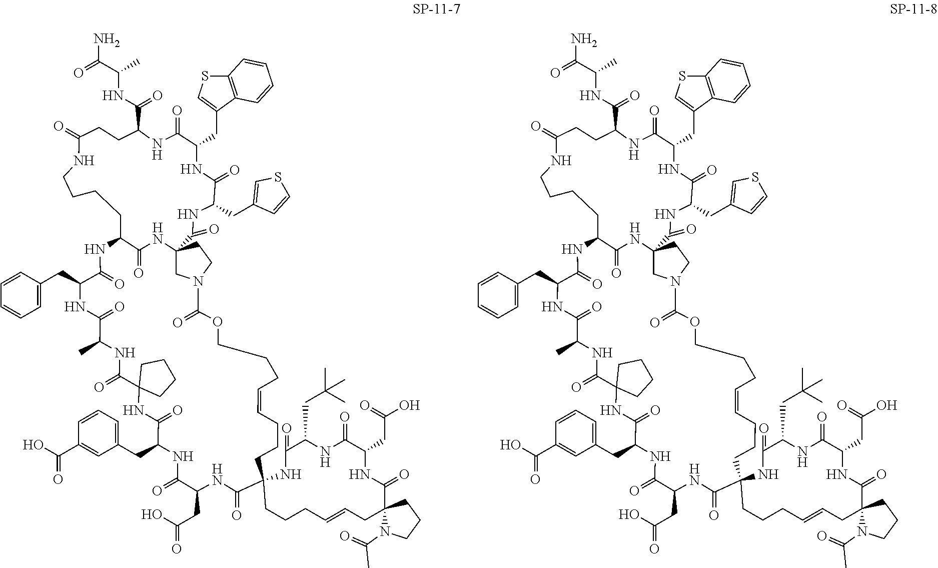

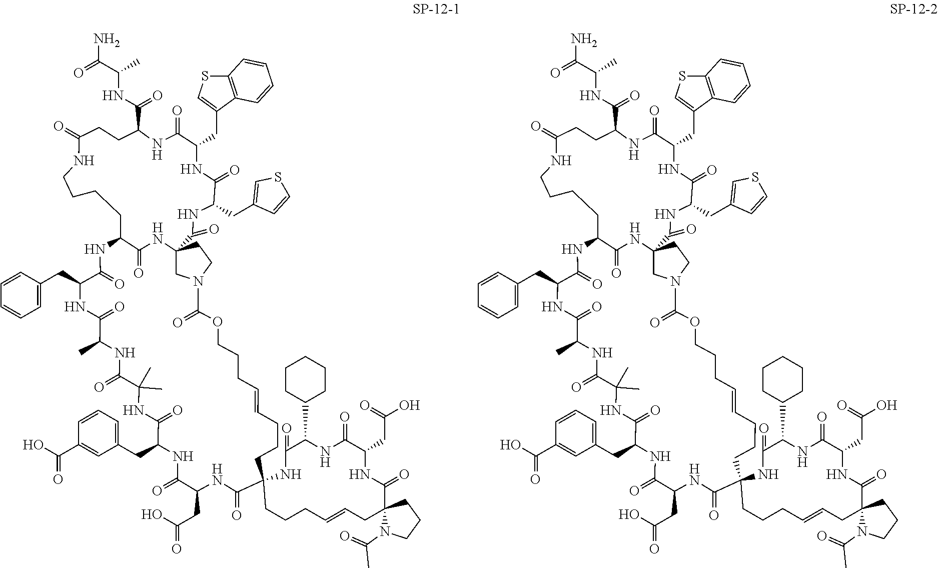

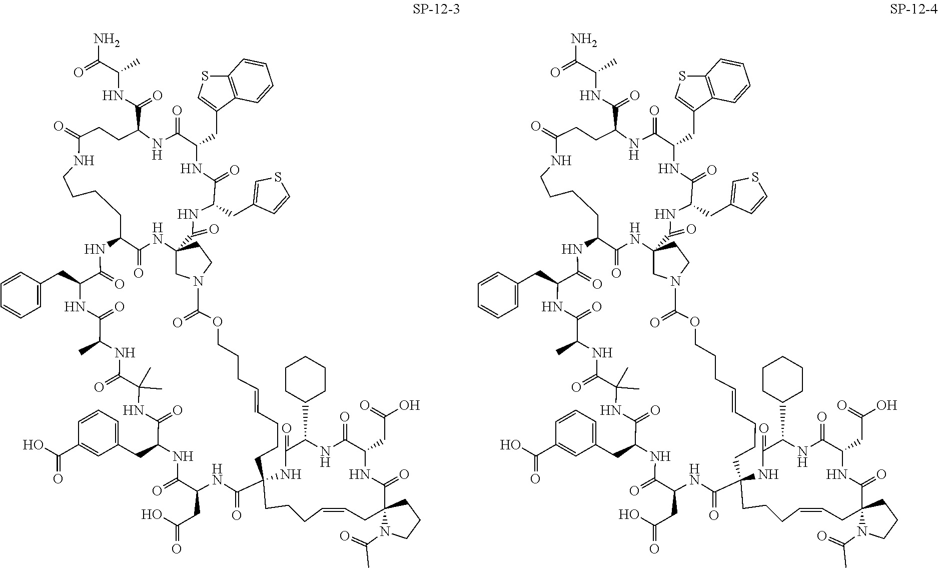

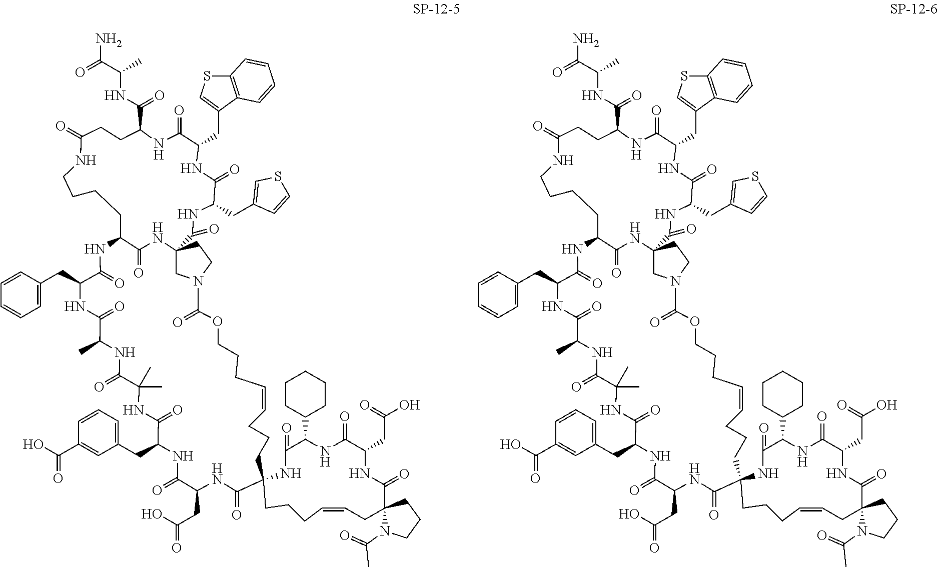

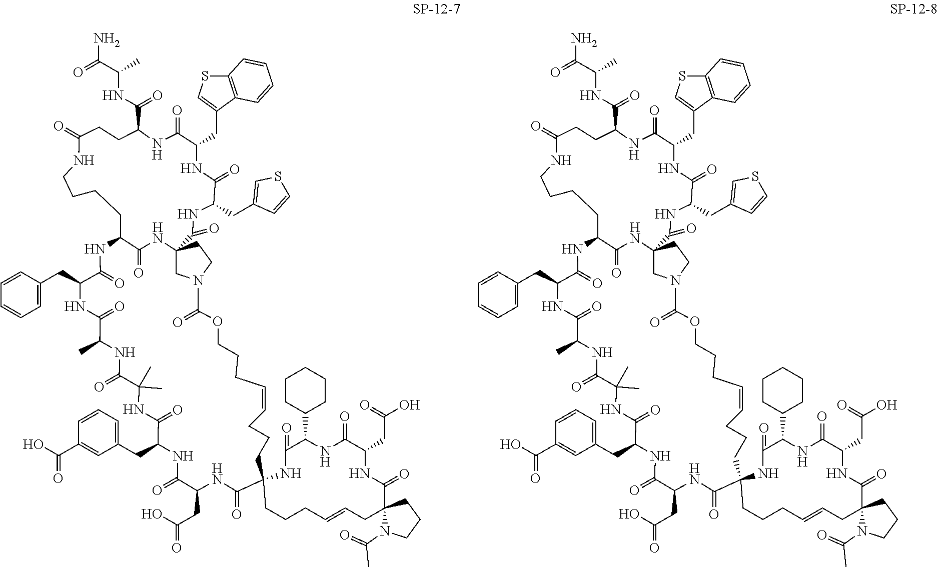

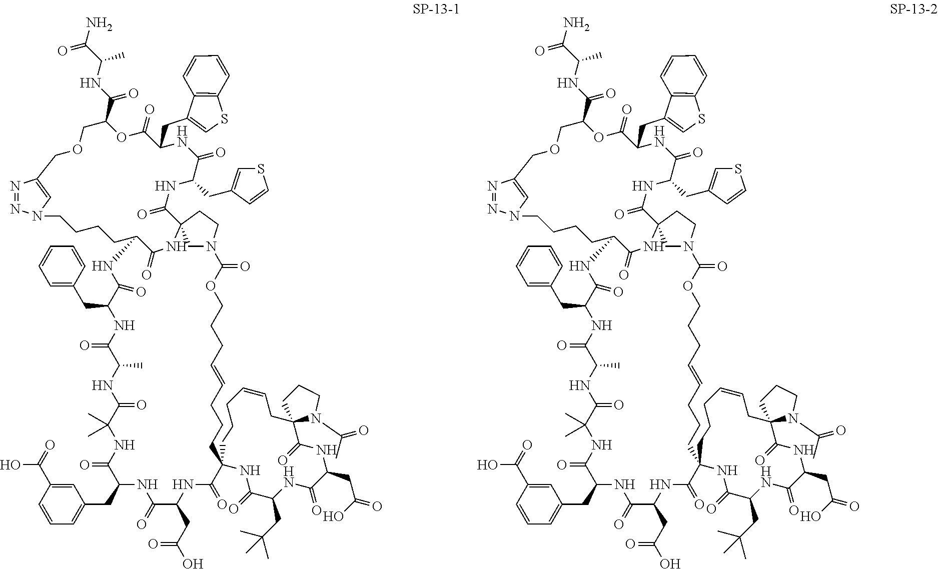

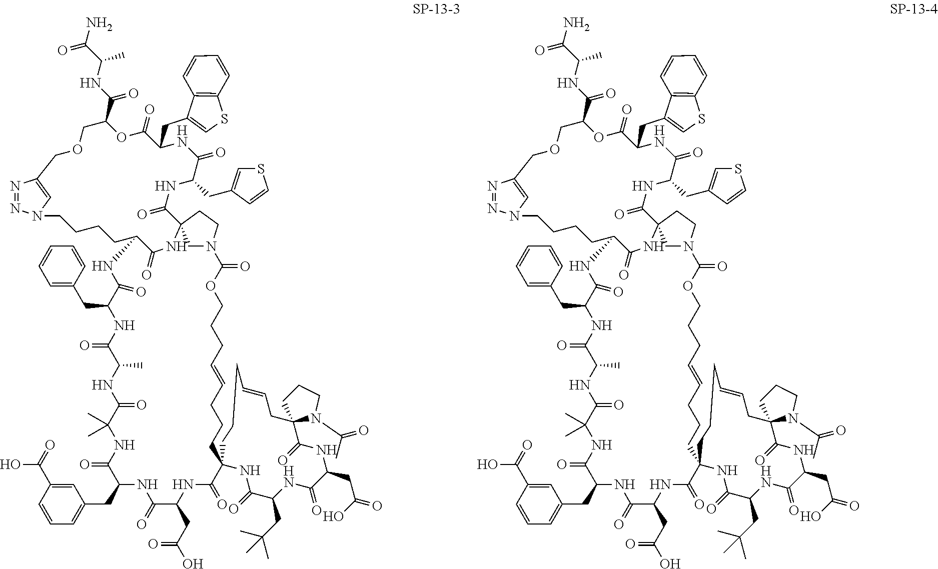

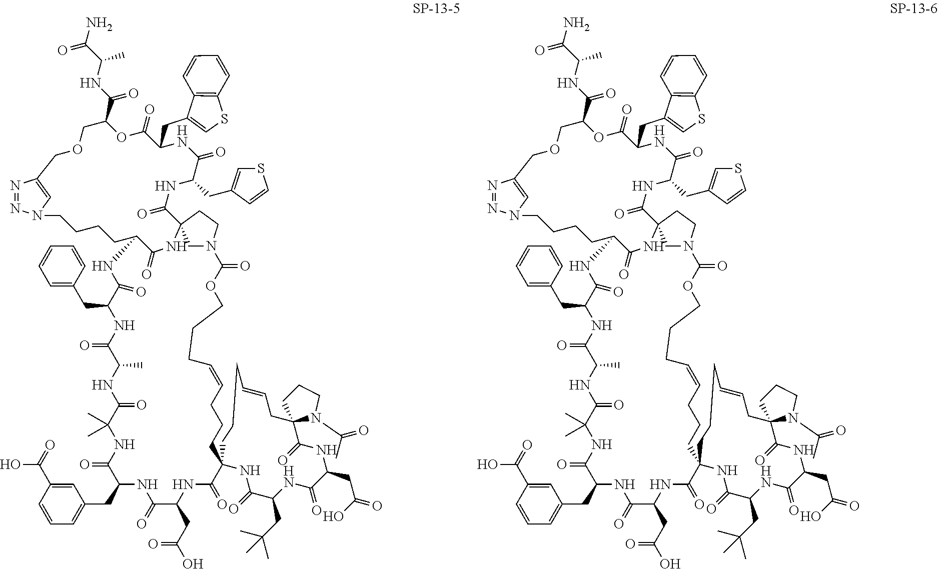

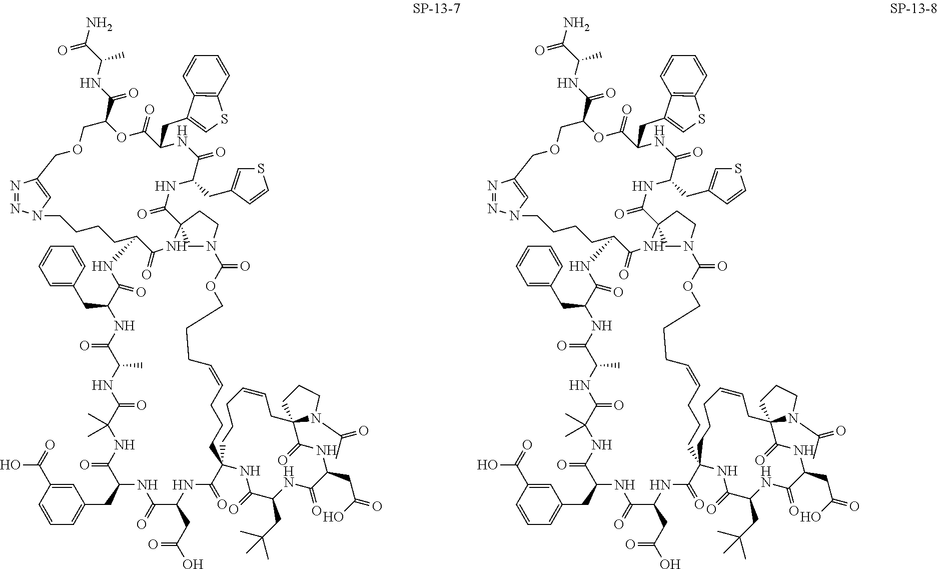

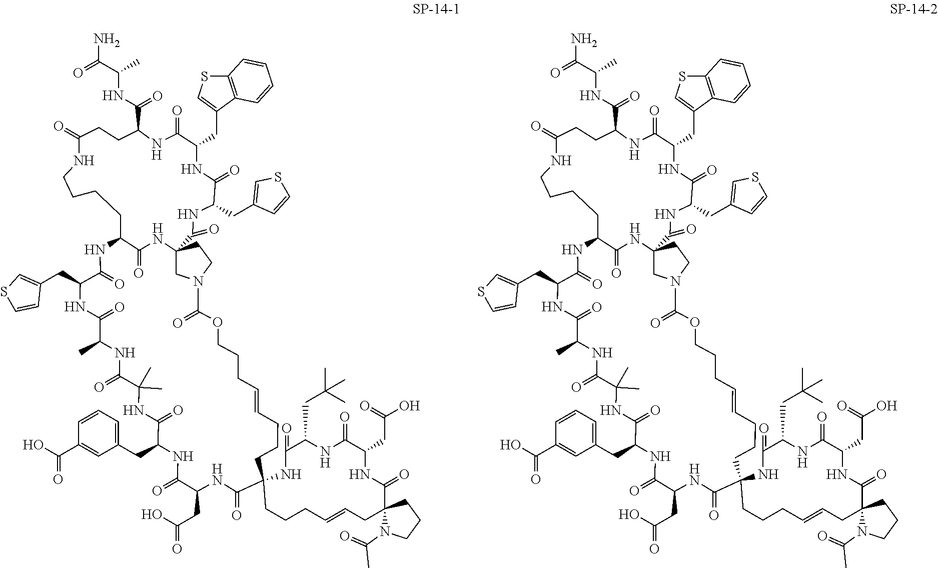

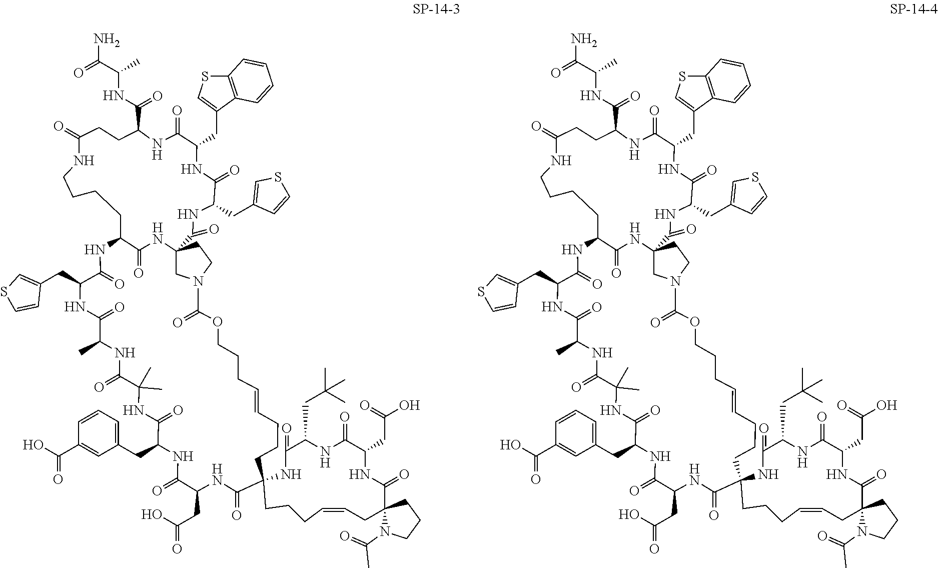

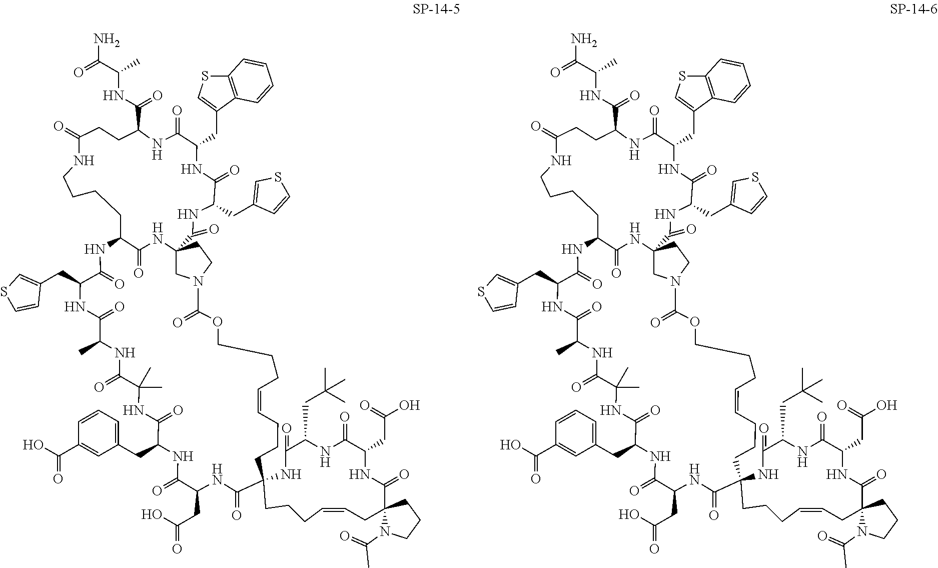

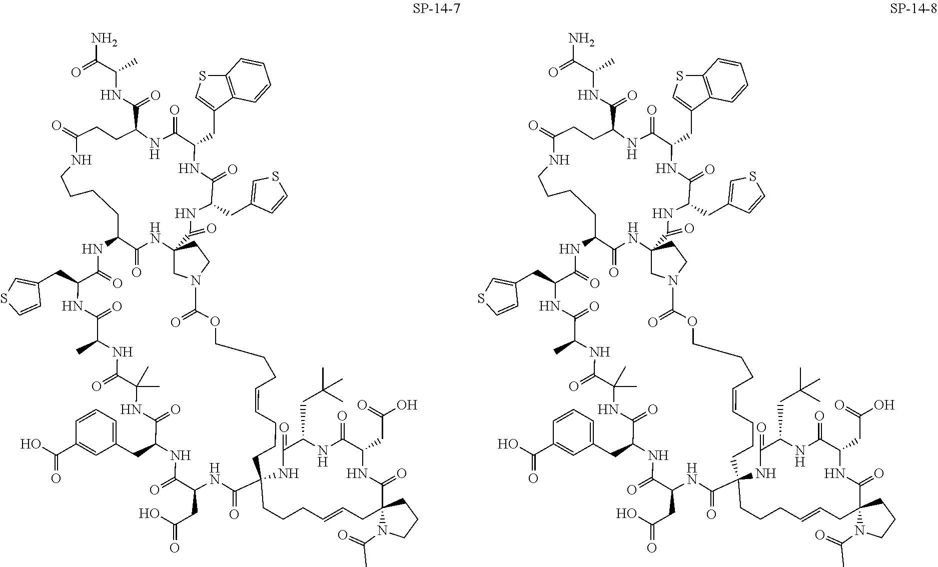

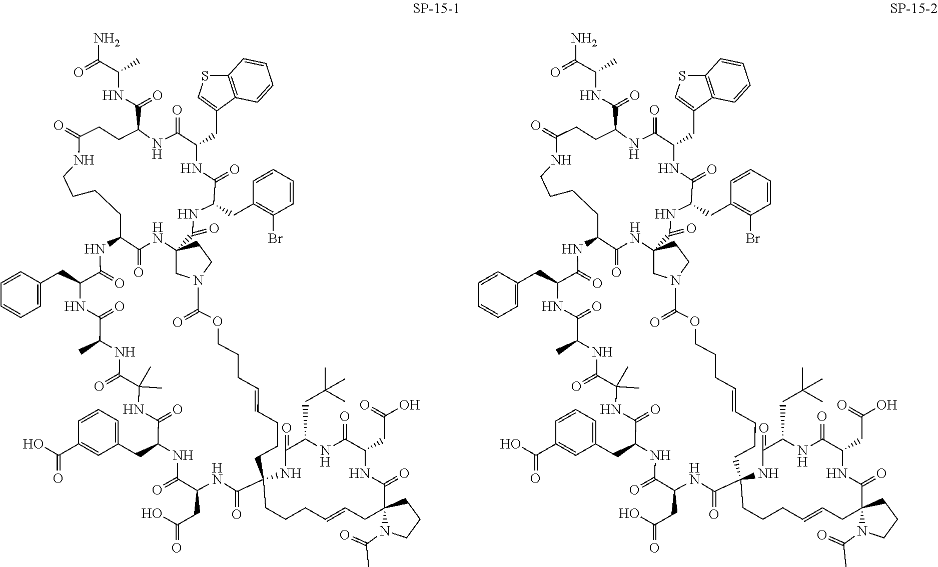

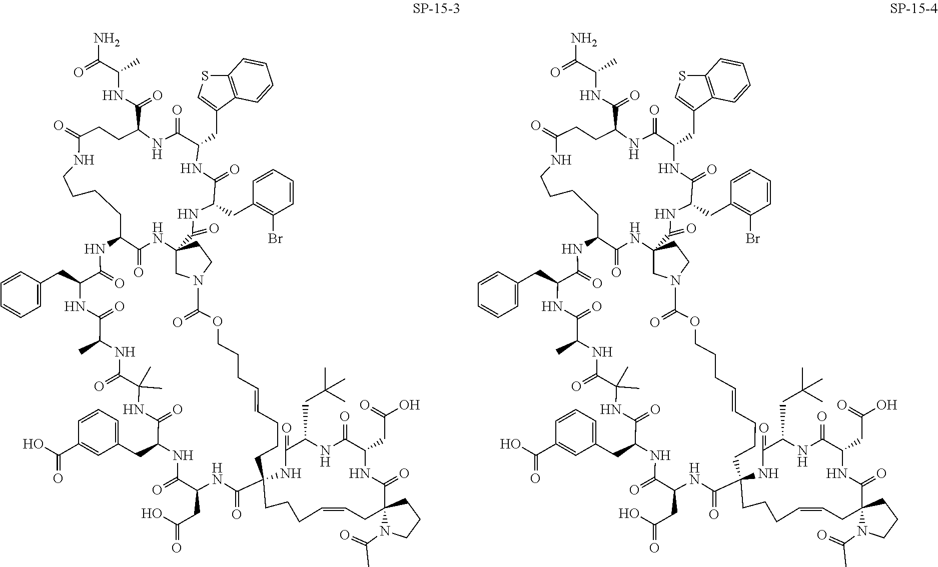

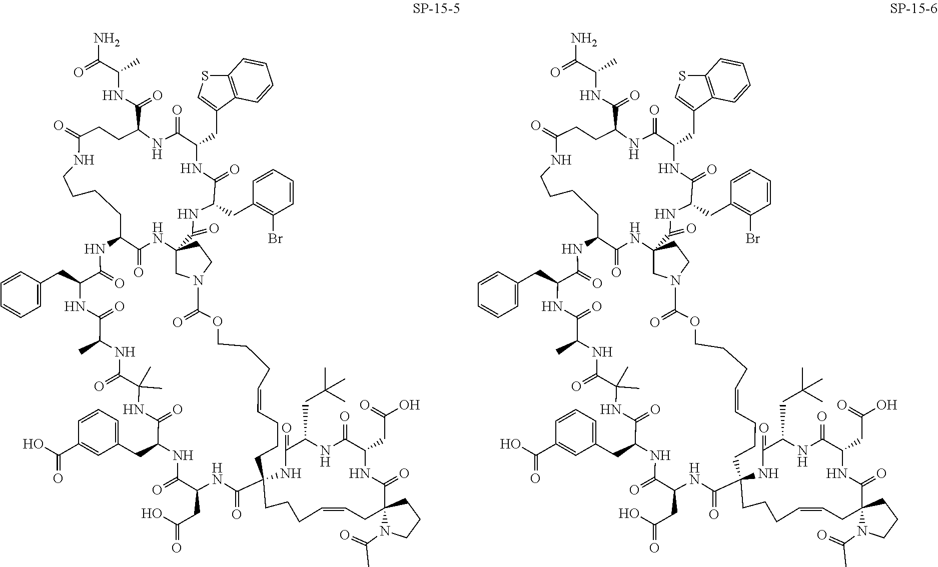

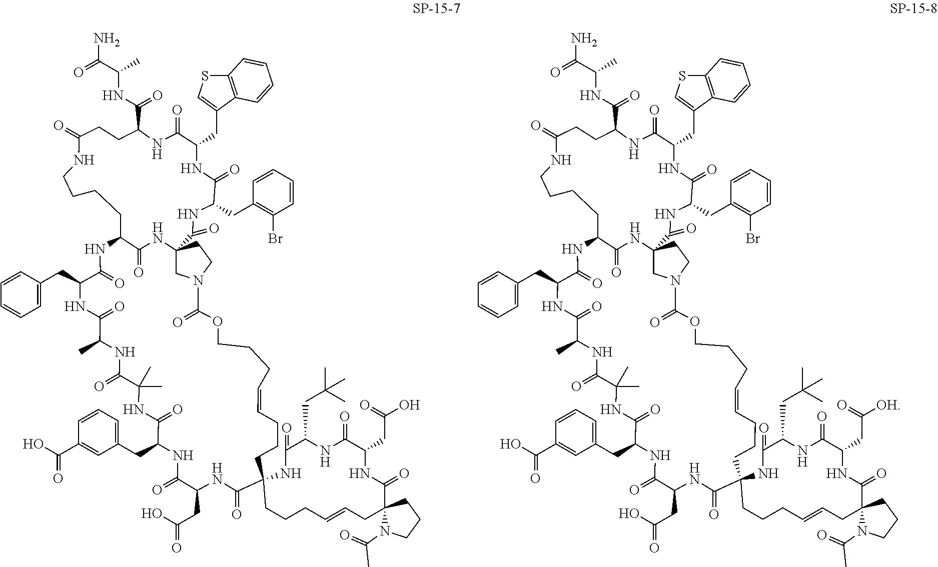

In some embodiments, an agent is SP-1-1 or a salt thereof. In some embodiments, an agent is SP-1-2 or a salt thereof. In some embodiments, an agent is SP-1-3 or a salt thereof. In some embodiments, an agent is SP-1-4 or a salt thereof. In some embodiments, an agent is SP-1-5 or a salt thereof. In some embodiments, an agent is SP-1-6 or a salt thereof. In some embodiments, an agent is SP-1-7 or a salt thereof. In some embodiments, an agent is SP-1-8 or a salt thereof. In some embodiments, an agent is SP-2-1 or a salt thereof. In some embodiments, an agent is SP-2-2 or a salt thereof. In some embodiments, an agent is SP-2-3 or a salt thereof. In some embodiments, an agent is SP-2-4 or a salt thereof. In some embodiments, an agent is SP-2-5 or a salt thereof. In some embodiments, an agent is SP-2-6 or a salt thereof. In some embodiments, an agent is SP-2-7 or a salt thereof. In some embodiments, an agent is SP-2-8 or a salt thereof. In some embodiments, an agent is SP-3-1 or a salt thereof. In some embodiments, an agent is SP-3-2 or a salt thereof. In some embodiments, an agent is SP-4-1 or a salt thereof. In some embodiments, an agent is SP-4-2 or a salt thereof. In some embodiments, an agent is SP-4-3 or a salt thereof. In some embodiments, an agent is SP-4-4 or a salt thereof. In some embodiments, an agent is SP-4-5 or a salt thereof. In some embodiments, an agent is SP-4-6 or a salt thereof. In some embodiments, an agent is SP-4-7 or a salt thereof. In some embodiments, an agent is SP-4-8 or a salt thereof. In some embodiments, an agent is SP-5-1 or a salt thereof. In some embodiments, an agent is SP-5-2 or a salt thereof. In some embodiments, an agent is SP-5-3 or a salt thereof. In some embodiments, an agent is SP-5-4 or a salt thereof. In some embodiments, an agent is SP-5-5 or a salt thereof. In some embodiments, an agent is SP-5-6 or a salt thereof. In some embodiments, an agent is SP-5-7 or a salt thereof. In some embodiments, an agent is SP-5-8 or a salt thereof. In some embodiments, an agent is SP-6 or a salt thereof. In some embodiments, an agent is SP-7-1 or a salt thereof. In some embodiments, an agent is SP-7-2 or a salt thereof. In some embodiments, an agent is SP-7-3 or a salt thereof. In some embodiments, an agent is SP-7-4 or a salt thereof. In some embodiments, an agent is SP-7-5 or a salt thereof. In some embodiments, an agent is SP-7-6 or a salt thereof. In some embodiments, an agent is SP-7-7 or a salt thereof. In some embodiments, an agent is SP-7-8 or a salt thereof. In some embodiments, an agent is SP-8-1 or a salt thereof. In some embodiments, an agent is SP-8-2 or a salt thereof. In some embodiments, an agent is SP-8-3 or a salt thereof. In some embodiments, an agent is SP-8-4 or a salt thereof. In some embodiments, an agent is SP-8-5 or a salt thereof. In some embodiments, an agent is SP-8-6 or a salt thereof. In some embodiments, an agent is SP-8-7 or a salt thereof. In some embodiments, an agent is SP-8-8 or a salt thereof. In some embodiments, an agent is SP-9-1 or a salt thereof. In some embodiments, an agent is SP-9-2 or a salt thereof. In some embodiments, an agent is SP-9-3 or a salt thereof. In some embodiments, an agent is SP-9-4 or a salt thereof. In some embodiments, an agent is SP-9-5 or a salt thereof. In some embodiments, an agent is SP-9-6 or a salt thereof. In some embodiments, an agent is SP-9-7 or a salt thereof. In some embodiments, an agent is SP-9-8 or a salt thereof. In some embodiments, an agent is SP-10-1 or a salt thereof. In some embodiments, an agent is SP-10-2 or a salt thereof. In some embodiments, an agent is SP-10-3 or a salt thereof. In some embodiments, an agent is SP-10-4 or a salt thereof. In some embodiments, an agent is SP-10-5 or a salt thereof. In some embodiments, an agent is SP-10-6 or a salt thereof. In some embodiments, an agent is SP-10-7 or a salt thereof. In some embodiments, an agent is SP-10-8 or a salt thereof. In some embodiments, an agent is SP-11-1 or a salt thereof. In some embodiments, an agent is SP-11-2 or a salt thereof. In some embodiments, an agent is SP-11-3 or a salt thereof. In some embodiments, an agent is SP-11-4 or a salt thereof. In some embodiments, an agent is SP-11-5 or a salt thereof. In some embodiments, an agent is SP-11-6 or a salt thereof. In some embodiments, an agent is SP-11-7 or a salt thereof. In some embodiments, an agent is SP-11-8 or a salt thereof. In some embodiments, an agent is SP-12-1 or a salt thereof. In some embodiments, an agent is SP-12-2 or a salt thereof. In some embodiments, an agent is SP-12-3 or a salt thereof. In some embodiments, an agent is SP-12-4 or a salt thereof. In some embodiments, an agent is SP-12-5 or a salt thereof. In some embodiments, an agent is SP-12-6 or a salt thereof. In some embodiments, an agent is SP-12-7 or a salt thereof. In some embodiments, an agent is SP-12-8 or a salt thereof. In some embodiments, an agent is SP-13-1 or a salt thereof. In some embodiments, an agent is SP-13-2 or a salt thereof. In some embodiments, an agent is SP-13-3 or a salt thereof. In some embodiments, an agent is SP-13-4 or a salt thereof. In some embodiments, an agent is SP-13-5 or a salt thereof. In some embodiments, an agent is SP-13-6 or a salt thereof. In some embodiments, an agent is SP-13-7 or a salt thereof. In some embodiments, an agent is SP-13-8 or a salt thereof. In some embodiments, an agent is SP-14-1 or a salt thereof. In some embodiments, an agent is SP-14-2 or a salt thereof. In some embodiments, an agent is SP-14-3 or a salt thereof. In some embodiments, an agent is SP-14-4 or a salt thereof. In some embodiments, an agent is SP-14-5 or a salt thereof. In some embodiments, an agent is SP-14-6 or a salt thereof. In some embodiments, an agent is SP-14-7 or a salt thereof. In some embodiments, an agent is SP-14-8 or a salt thereof. In some embodiments, an agent is SP-15-1 or a salt thereof. In some embodiments, an agent is SP-15-2 or a salt thereof. In some embodiments, an agent is SP-15-3 or a salt thereof. In some embodiments, an agent is SP-15-4 or a salt thereof. In some embodiments, an agent is SP-15-5 or a salt thereof. In some embodiments, an agent is SP-15-6 or a salt thereof. In some embodiments, an agent is SP-15-7 or a salt thereof. In some embodiments, an agent is SP-15-8 or a salt thereof.

Agents, e.g., peptides including stapled peptides, can contain various numbers of amino acid residues. In some embodiments, a length of a peptide agent is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acid residues. In some embodiments, a length is about 10 amino acid residues. In some embodiments, a length is about 11 amino acid residues. In some embodiments, a length is about 12 amino acid residues. In some embodiments, a length is about 13 amino acid residues. In some embodiments, a length is about 14 amino acid residues. In some embodiments, a length is about 15 amino acid residues. In some embodiments, a length is about 16 amino acid residues. In some embodiments, a length is about 17 amino acid residues. In some embodiments, a length is about 18 amino acid residues. In some embodiments, a length is about 19 amino acid residues. In some embodiments, a length is about 20 amino acid residues.

In some embodiments, as described herein, one or more staples independently comprise an olefin double bond (e.g., formed through olefin metathesis). In some embodiments, one or more staples independently comprise an amide group (e.g., formed through amidation). In some embodiments, at least one staple does not contain an olefin double bond. In some embodiments, there is at least one staple whose formation does not comprise reactions of olefins such as olefin metathesis and/or modification of olefin double bonds (e.g., hydrogenation, epoxidation, etc.).

In some embodiments, a residue of a staple (e.g., B5) is so positioned that if its position is P (e.g., X⁴) a first acidic amino acid residue is at position P-2 (e.g., X²), a second acidic amino acid residue is positioned at P+1 (e.g., X⁵), a third acidic amino acid residue is positioned at P+2 (e.g., X⁶), a hydrophobic amino acid residue is positioned at P+4 (e.g., X⁸), a first aromatic amino acid residue is positioned at P+5 (e.g., X⁹), a second aromatic amino acid residue is positioned at P+8 (e.g., X¹²), and/or a third aromatic amino acid residue is positioned at P+9 (e.g., X¹³). In some embodiments, a staple is a (i, i+7) staple, and the other residue of the staple is positioned at P+7 (e.g., X¹). In some embodiments, a first acidic amino acid residue is at position P-2 (e.g., X²). In some embodiments, a second acidic amino acid residue is positioned at P+1 (e.g., X⁵). In some embodiments, a third acidic amino acid residue is positioned at P+2 (e.g., X⁶). In some embodiments, a hydrophobic amino acid residue is positioned at P+4 (e.g., X⁸). In some embodiments, a first aromatic amino acid residue is positioned at P+5 (e.g., X⁹). In some embodiments, a second aromatic amino acid residue is positioned at P+8 (e.g., X²). In some embodiments, a third aromatic amino acid residue is positioned at P+9 (e.g., X¹³). In some embodiments, a first acidic amino acid residue is at position P-2 (e.g., X²) a second acidic amino acid residue is positioned at P+1 (e.g., X⁵), a first aromatic amino acid residue is positioned at P+5 (e.g., X⁹), a second aromatic amino acid residue is positioned at P+8 (e.g., X¹²), and a third aromatic amino acid residue is positioned at P+9 (e.g., X¹³). In some embodiments, a first acidic amino acid residue is at position P-2 (e.g., X²), a second acidic amino acid residue is positioned at P+1 (e.g., X⁵), a third acidic amino acid residue is positioned at P+2 (e.g., X⁶), a hydrophobic amino acid residue is positioned at P+4 (e.g., X⁸), a first aromatic amino acid residue is positioned at P+5 (e.g., X⁹), a second aromatic amino acid residue is positioned at P+8 (e.g., X²), and a third aromatic amino acid residue is positioned at P+9 (e.g., X¹³). In some embodiments, a stapled peptide agent comprises acidic amino acid residues at positions P-2 and P+1, and aromatic amino acid residues at positions P+5, P+8 and P+9. In some embodiments, a stapled peptide agent comprises acidic amino acid residues at positions P-2, P+1 and P+2, and aromatic amino acid residues at positions P+5, P+8 and P+9. In some embodiments, a stapled peptide agent comprises acidic amino acid residues at positions P-2 and P+1, a hydrophobic amino acid residue at position P+4, and aromatic amino acid residues at positions P+5, P+8 and P+9. In some embodiments, a stapled peptide agent comprises acidic amino acid residues at positions P-2, P+1 and P+2, a hydrophobic amino acid residue at position P+4, and aromatic amino acid residues at positions P+5, P+8 and P+9. In some embodiments, P is 3. In some embodiments, P is 4. In some embodiments, P is 5. In some embodiments, P is 6. In some embodiments, P is 7. In some embodiments, an amino acid residue at position P comprises two groups for stapling, e.g., B4, B5, B6, etc. In some embodiments, it is B4. In some embodiments, it is B5. In some embodiments, it is B6. In some embodiments, an agent comprises a staple and a first additional staple, e.g., a (i, i+3) or (i, i+4) staple. In some embodiments, a staple and a first additional staple are bonded to the same residue (e.g., B5, B6, etc.). In some embodiments, the other residue of a first additional residue is at position P-2 (e.g., when a moiety for stapling like a terminal olefin is in a P-terminal group which is considered a portion of X¹), P-3 or P-4. In some embodiments, an agent comprises a second additional staple, e.g., a (i, i+4) staple (e.g., stapling residues at positions P+6 (e.g., X¹⁰) and P+10 (e.g., X¹⁴), a (i, i+3) staple (e.g., stapling residues at positions P+3 (e.g., X⁷) and P+6 (e.g., X¹⁰), a (i, i+7) staple (e.g., stapling residues at positions P+3 (e.g., X⁷) and P+10 (e.g., X¹⁴), etc.). In some embodiments, an agent comprises a second additional staple which is a (i, i+4) staple stapling residues at positions P+6 (e.g., X¹⁰) and P+10 (e.g., X¹⁴). In some embodiments, an agent comprises a third additional staple, e.g., a (i, i+4) staple stapling residues at positions P-1 (e.g., X³) and P+3 (e.g., X⁷). In some embodiments, there are three staples in a stapled peptide agent. In some embodiments, there are four staples in a stapled peptide agent. As demonstrated herein, stapled agents comprising so positioned staples and residues can provide various desired properties and activities. In some embodiments, positioning of one or more staples may be shifted relevant to various acidic, hydrophobic and/or aromatic amino acid residues described herein, e.g., in some embodiments, stapled peptide agents comprise stapled residues at position P and P+7 (and optionally P-3 or P-4), acidic amino acid residues are at positions P-1, and P+2, and aromatic amino acid residues at positions P+6, P+9 and P+10, and optionally an acid amino acid residue at P+3 and/or a hydrophobic amino acid residue at positon P+5. It was observed that various stapled peptide agents with shifted staples can bind to beta-catenin when assessed by fluorescence polarization.

Certain useful staples are described in the “Agents” section, below.

### Beta-Catenin

Among other things, the present disclosure provides technologies for modulating one or more beta-catenin functions. In some embodiments, the present disclosure provides useful technologies for inhibiting one or more beta-catenin functions that are associated with cancer or hyperplasia. In some embodiments, provided technologies are useful for preventing and treating conditions, disorders or diseases whose prevention and/or treatment will benefits from inhibition of beta-catenin. In some embodiments, a condition, disorder or disease is cancer.

Beta-catenin is reported to have various functions. For example, it can regulate and coordinate transcription of various genes. It is reported that high beta-catenin activity and/or expression levels may contribute to the development various conditions, disorders or diseases including cancer. Mutations and overexpression of beta-catenin are reported to be associated with conditions, disorders or diseases including many cancers including colorectal cancer, lung cancer, and breast cancer. Dysregulation of the Wnt/0-catenin signaling pathway has reportedly been linked to a number of conditions, disorders or diseases, including neurodegenerative diseases, psychiatric diseases, cancers, asthma, and even wound healing. An abundance of published research, both clinical and preclinical, has indicated that hyperactivated Wnt/beta-catenin activity drives tumorigenesis and is required for tumor maintenance in various cancers. Many Wnt inhibitors largely modulate this pathway at the extracellular ligand/receptor level, e.g., by preventing Wnt ligand secretion or by blocking Wnt ligand interaction with its receptors at the plasma membrane. It has been reported that many activating Wnt pathway mutations are found in APC and/or CTNNB1, which are downstream of membrane-proximal events. Among other things, the present disclosure encompasses the recognition that many agents at the extracellular ligand/receptor level are insufficient to treat many relevant patients, e.g., those with downstream mutations/abnormalities. In some embodiments, Wnt pathway-activating mutations converge on beta-catenin/TCF node. In some embodiments, the present disclosure targets beta-catenin/TCF interaction, e.g., as a therapeutic approach. Agents that can modulate beta-catenin functions are useful for various purposes including preventing and/or treating various conditions, disorders or diseases associated with beta-catenin.

### Binding Sites

Beta-catenin may interact with various agents at various binding sites each independently comprising a set of amino acid residues that interact with binding agents. For example, certain binding sites are utilized for beta-catenin interactions with Axin, APC, C-cadherin, E-cadherin, TCF3, and Bcl9. For interactions with TCF3, it has been reported that two or more binding sites may be utilized simultaneously to interact with different portions of TCF3. See, e.g., Graham et al. Cell, Vol. 103, 885-896, 2000.

In some embodiments, provided agents bind to beta-catenin at a unique binding site. In some embodiments, provided agents interact with beta-catenin at a set of amino acid residues that are different from previously reported binding sites, e.g., those for Axin, APC, C-cadherin, E-cadherin, TCF3 or Bcl9.

For example, in some embodiments, provided agents interact with one or more or all (e.g., about 1-23, 1-20, 1-15, 1-10, 1-5, 5-23, 5-20, 5-15, 5-10, 6-23, 6-20, 6-15, 6-10, 7-23, 7-20, 7-15, 7-10, 8-23, 8-20, 8-15, 8-10, 9-23, 9-20, 9-15, 9-10, 10-23, 10-20, 10-15, 11-23, 11-20, 11-15, 12-23, 12-20, 12-15, 13-23, 13-20, 13-15, 13-23, 14-20, 15-23, 15-20, 16-23, 16-20, 17-23, 17-20, 18-23, or 18-20, or about 1, 2, 3, 4, 5, 6, 7, 8, 9, 10, 11, 12, 13, 14, 15, 16, 17, 18, 19, 20, 21, 22, or 23, etc.) of a set of amino acid residues that are or correspond to amino acid residues in SEQ ID NO: 1, e.g., in some embodiments, the following amino acid residues of SEQ ID NO: 1: A305, Y306, G307, N308, Q309, K312, R342, K345, V346, V349, Q375, R376, Q379, N380, L382, W383, R386, N387, D413, N415, V416, T418, and C419. In some embodiments, a set of amino acid residues are or correspond to amino acid residues A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, a set of amino acid residues are or correspond to amino acid residues A305, Y306, G307, N308, Q309, K312, K345, V346, V349, Q379, N380, L382, W383, R386, N387, D413, N415, V416, T418, and C419 of SEQ ID NO: 1. In some embodiments, a set of amino acid residues are or correspond to amino acid residues G307, K312, K345, W383, N387, D413, and N415 of SEQ ID NO: 1. In some embodiments, a set of amino acid residues are or correspond to amino acid residues G307, K312, K345, Q379, L382, W383, N387, N415 and V416 of SEQ ID NO: 1. In some embodiments, a set of amino acid residues are or correspond to amino acid residues Y306, G307, K312, K345, Q379, L382, W383, N387, N415 and V416 of SEQ ID NO: 1. In some embodiments, a set of amino acid residues are or correspond to amino acid residues G307, K312, K345, Q379, L382, W383, R386, N387, N415 and V416 of SEQ ID NO: 1. In some embodiments, a set of amino acid residues are or correspond to amino acid residues Y306, G307, K312, K345, Q379, L382, W383, R386, N387, N415 and V416 of SEQ ID NO: 1. In some embodiments, a set of amino acid residues are or correspond to amino acid residues Y306, G307, K312, K345, V349, Q379, L382, W383, N387, N415 and V416 of SEQ ID NO: 1. In some embodiments, a set of amino acid residues are or correspond to amino acid residues Y306, G307, K312, K345, V349, Q379, L382, W383, R386, N387, N415 and V416 of SEQ ID NO: 1. In some embodiments, a set of amino acid residues are or correspond to amino acid residues G307, K312, K345, W383, and N387 of SEQ ID NO: 1. In some embodiments, a set of amino acid residues are or correspond to amino acid residues Y306, G307, K312, R386 and N387 of SEQ ID NO: 1. In some embodiments, provided agents interact with Y306 or an amino acid residue corresponding thereto. In some embodiments, provided agents interact with G307 or an amino acid residue corresponding thereto. In some embodiments, provided agents interact with K312 or an amino acid residue corresponding thereto. In some embodiments, provided agents interact with K345 or an amino acid residue corresponding thereto. In some embodiments, provided agents interact with V349 or an amino acid residue corresponding thereto. In some embodiments, provided agents interact with Q379 or an amino acid residue corresponding thereto. In some embodiments, provided agents interact with L382 or an amino acid residue corresponding thereto. In some embodiments, provided agents interact with W383 or an amino acid residue corresponding thereto. In some embodiments, provided agents interact with R386 or an amino acid residue corresponding thereto. In some embodiments, provided agents interact with N387 or an amino acid residue corresponding thereto. In some embodiments, provided agents interact with N415 or an amino acid residue corresponding thereto. In some embodiments, provided agents interact with V416 or an amino acid residue corresponding thereto.

In some embodiments, a present agent interacts with a polypeptide whose sequence corresponds to aa 146-aa665 of human beta-catenin. In some embodiments, a present agent interacts with a polypeptide whose sequence comprises or is SEQ ID NO: 2:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

In some embodiments, all amino acid residues that interact with a provided agent is with SEQ ID NO: 2. In some embodiments, amino acid residues that interact with a provided agent (e.g., one or more amino acid residues in an agent) interacts with an agent through hydrogen bonding, hydrophobic interactions or salt bridge. As appreciated by those skilled in the art, when two amino acid residues interacting with each other, they are typically within a certain range of distances when, e.g., assessed using crystallography, NMR, etc.

In some embodiments, certain amino acid residues reported to interact with one or more polypeptides are not significantly involved in interactions between provided and beta-catenin. In some embodiments, provided agents do not interact with an Axin binding site. In some embodiments, provided agents do not interact with a Bcl9 binding site. In some embodiments, provided agents do not interact with one or more or all of amino acid residues that are or correspond to N426, C429, K435, R469, H470, S473, R474, K508 and N516 of SEQ ID NO: 1. In some embodiments, provided agents do not interact with N426 or an amino acid residue corresponding thereto. In some embodiments, provided agents do not interact with C429 or an amino acid residue corresponding thereto. In some embodiments, provided agents do not interact with K435 or an amino acid residue corresponding thereto. In some embodiments, provided agents do not interact with R469 or an amino acid residue corresponding thereto. In some embodiments, provided agents do not interact with H470 or an amino acid residue corresponding thereto. In some embodiments, provided agents do not interact with S473 or an amino acid residue corresponding thereto. In some embodiments, provided agents do not interact with R474 or an amino acid residue corresponding thereto. In some embodiments, provided agents do not interact with K508 or an amino acid residue corresponding thereto. In some embodiments, provided agents do not interact with N516 or an amino acid residue corresponding thereto.

In some embodiments, mutation of one or more amino acid residues outside of SEQ ID NO: 2 in beta-catenin does not significant/y (e.g., not exceeding 30%, 40%, 50%, 60%, 70%, 75%, 80%, 85%, 90% or more) reduce interactions of beta-catenin with a provided agent. In some embodiments, mutation of one or more or all of amino acid residues that are or correspond to N426, C429, K435, R469, H470, S473, R474, K508 and N516 of SEQ ID NO: 1 does not significantly reduce interactions of beta-catenin with a provided agent. In some embodiments, mutation of N426 or an amino acid residue corresponding thereto does not significantly reduce interaction of beta-catenin with an agent. In some embodiments, mutation of Q379 or an amino acid residue corresponding thereto (e.g., to Ala, Glu, Phe, Trp, etc.) does not significantly reduce interaction of beta-catenin with an agent.

In some embodiments, an agent binds to a TCF site of beta-catenin. In some embodiments, an agent interacts with one or more but not all amino acid residues that interact with TCF. In some embodiments, an agent interacts with one or more but not all amino acid residues that interact with an extended region of XTcf3-CBD. In some embodiments, an agent does not interact with beta-catenin amino acid residues that interact with a beta-hairpin module of XTcf3-CBD. In some embodiments, an agent does not interact with beta-catenin amino acid residues that interact with a helix module of XTcf3-CBD. For certain amino acid residues that interact various modules of XTcF3-CBD, see, e.g., Graham et al. Cell, Vol. 103, 885-896, 2000.

In some embodiments, an agent competes with TCF for beta-catenin binding. In some embodiments, an agent competes with an extended region of TCF (e.g., Ala14-Glu24, or Asp16-Glu24, as described in Graham et al. Cell, Vol. 103, 885-896, 2000) for beta-catenin binding. In some embodiments, compared to an extended region of TCF, an agent does not compete, or competes at a less degree, with Axin for beta-catenin binding. In some embodiments, compared to an extended region of TCF, an agent does not compete, or competes at a less degree, with Bcl9 for beta-catenin binding. In some embodiments, compared to an extended region of TCF, an agent does not compete, or competes at a less degree, with a beta-hairpin module of XTcf3-CBD for beta-catenin binding. In some embodiments, compared to an extended region of TCF, an agent does not compete, or competes at a less degree, with a helix module of XTcf3-CBD for beta-catenin binding. In some embodiments, an agent competes with E-cadherin for beta-catenin binding.

In some embodiments, the present disclosure provides complexes of peptides (e.g., polypeptides whose sequences are or comprises SEQ ID NO: 1 or 2) and provided agents. In some embodiments, in such complexes polypeptides and provided agents interact with one or more or all amino acid residues as described herein, and optionally do not interact with one or more or all amino acid residues as described herein.

In some embodiments, the present disclosure provides complexes comprising a provided agent and a beta-catenin polypeptide or a portion thereof. In some embodiments, a portion thereof comprises one or more or all of the interacting residues as described herein. In some embodiments, an agent and a beta-catenin polypeptide or a portion thereof interact with other at one or more or all of the interacting residues.

### Certain Agents

In some embodiments, the present disclosure provides an agent having the structure of formula I:

R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue; L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, the present disclosure provides an agent having the structure of formula I:

R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}, wherein R^{AA1} is CO₂R or —SO₂R;
L^{AA2} is L^{AR}, wherein a methylene unit is replaced with —C(R′)(R^{AS})—, wherein R^{AS} is -L^{AS2}-R^{AA2} wherein R^{AA2} is —CO₂R or —SO₂R;
L^{AA3} is L^{AR}, wherein a methylene unit is replaced with —C(R′)(R^{AS})—, wherein R^{AA}s is -L^{AS3}-R^{AA3} wherein R^{AA3} is R′;
L^{AA4} is L^{AR}, wherein a methylene unit is replaced with —C(R′)(R^{AS})—, wherein R^{AA}s is -L^{AS4}-R^{AA4} wherein R^{AA}A4 is an optionally substituted group selected from 6-14 membered aryl or 5-14 membered heteroaryl having 1-6 heteroatoms;
L^{AA5} is L^{AR}, wherein a methylene unit is replaced with —C(R′)(R^{AS})—, wherein R^{AS} is -L^{AS}-R^{AA5} wherein R^{AA}A5 is an optionally substituted group selected from 6-14 membered aryl or 5-14 membered heteroaryl having 1-6 heteroatoms;
L^{AA6} is L^{AR}, wherein a methylene unit is replaced with —C(R′)(R^{AS})—, wherein R^{AS} is -L^{AS6}-R^{AA6} wherein R^{AA}A6 is an optionally substituted group selected from 6-14 membered aryl or 5-14 membered heteroaryl having 1-6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{AR} is independently an optionally substituted, bivalent C₁-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AS} is independently -L^{AS}-R′;
each L^{AS} is independently an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, a second R′ group and a third R′ group are attached to the same atom. In some embodiments, none of the first, second and fourth R′ groups are attached to the same atom. In some embodiments, none of the first, second, fourth, fifth and sixth R′ groups are attached to the same atom. In some embodiments, none of the first, second, fourth, fifth, sixth, seventh and eighth R′ groups are attached to the same atom. In some embodiments, each of the first, second, third and fourth R′ groups is independently attached to a different atom. In some embodiments, each of the first, second, third, fourth, fifth and sixth R′ groups is independently attached to a different atom. In some embodiments, each of the first, second, third, fourth, fifth, sixth, seventh and eighth R′ groups is independently attached to a different atom.

In some embodiments, a compound of formula I is a stapled peptide as described herein.

In some embodiments, each L^{s} is independently a staple as described herein. In some embodiments, L^{s}, e.g., L^{s} formed by taking a first and a second R′ groups, has a length of 5-20 (e.g., 5, 6, 7, 8, 9, 10, 11, 12, 13, 14, 15, 16, 17, 18, 19, or 20) atoms. Unless specified otherwise, a length between two connection sites, e.g., of L^{s}, L, etc., is the shortest covalent connection from one site to the other. For example, the length of —CH₂—CH₂— is 2 atoms (—C—C—), the length of 1, 3-phenylene is 3 atoms. In some embodiments, L^{s}, e.g., L^{s} formed by taking a third and a fourth R′ groups, has a length of 5-20 (e.g., 5, 6, 7, 8, 9, 10, 11, 12, 13, 14, 15, 16, 17, 18, 19, or 20) atoms. In some embodiments, L^{s}, e.g., L^{s} formed by taking a fifth and a sixth R′ groups, has a length of 5-20 (e.g., 5, 6, 7, 8, 9, 10, 11, 12, 13, 14, 15, 16, 17, 18, 19, or 20) atoms. In some embodiments, L^{s}, e.g., L^{s} formed by taking a seventh and an eighth R′ groups, has a length of 5-20 (e.g., 5, 6, 7, 8, 9, 10, 11, 12, 13, 14, 15, 16, 17, 18, 19, or 20) atoms.

Those skilled in the art reading the present disclosure will appreciate that staples, e.g., L^{s}, connecting two atoms having a longer distance typically has a longer length than staples connecting two atom having a shorter distance, e.g., (i, i+7) staples typically have longer lengths than (i, i+3) or (i, i+4) staples. In some embodiments, a length is 5 atoms. In some embodiments, a length is 6 atoms. In some embodiments, a length is 7 atoms. In some embodiments, a length is 8 atoms. In some embodiments, a length is 9 atoms. In some embodiments, a length is 10 atoms. In some embodiments, a length is 11 atoms. In some embodiments, a length is 12 atoms. In some embodiments, a length is 13 atoms. In some embodiments, a length is 14 atoms. In some embodiments, a length is 15 atoms. In some embodiments, a length is 16 atoms. In some embodiments, a length is 17 atoms. In some embodiments, a length is 18 atoms. In some embodiments, a length is 19 atoms. In some embodiments, a length is 20 atoms.

### L^{P1}

In some embodiments, L^{P1} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P1} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P1} is or comprises an amino acid residue. In some embodiments, L^{P1} is or comprises a peptide.

In some embodiments, L^{P1} is or comprises —[X]ₚ—X¹—, wherein each of p, X and X¹ is independently as described herein, and X¹ is bonded to L^{AA1}. In some embodiments, L^{P1} is or comprises —X¹—.

In some embodiments, L^{P1} comprises a —C(R′)₂— group, wherein one of the R′ groups is a first R′ group of the four. In some embodiments, such a —C(R′)₂— group is of an amino acid residue. In some embodiments, such a —C(R′)₂— group is of X¹. In some embodiments, such a carbon atom is an alpha carbon of an amino acid residue.

### L^{AA1}

In some embodiments, L^{AA1} is or comprises amino acid residue. In some embodiments, L^{AA1} is or comprises an amino acid residue that comprises a side chain comprising an acidic or polar group. In some embodiments, L^{AA1} is an amino acid residue that comprises a side chain comprising an acidic group.

In some embodiments, L^{AA1} is L^{AR}, wherein a methylene unit is replaced with —C(R′)(R^{AS})—, wherein each variable is independently as described herein. In some embodiments, L^{AA} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA1} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA1} is N(R′)—C(R′)(R^{AS})C(O)—, wherein each variable is independently as described herein. In some embodiments, L^{AA1} is NH—C(R′)(R^{AS})C(O)—, wherein each variable is independently as described herein.

In some embodiments, L^{AS1} is L^{AS} as described herein. In some embodiments, R^{AA1} is —CO₂R, wherein R is as described herein. In some embodiments, R is H. In some embodiments, L^{AA1} is a residue of an acidic amino acid residue, e.g., Asp, Glu, etc. In some embodiments, L^{AA1} is X² as described herein.

### L^{P2}

In some embodiments, L^{P2} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P2} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P2} is or comprises an amino acid residue. In some embodiments, L^{P2} is or comprises a peptide.

In some embodiments, L^{P2} is or comprises —[X]pX⁴[X]p′-, wherein each of p, p′, X and X⁴ is independently as described herein. In some embodiments, L^{P2} is or comprises —[X]pX³X⁴[X]p′-, wherein each X, X³ and X⁴ is independently an amino acid residue, and each of p and p′ is independently 0-10. In some embodiments, L^{P2} is or comprises —X³X⁴—, wherein each X³ and X⁴ is independently as described herein, and X⁴ is bonded to L^{AA2}.

In some embodiments, L^{P2} comprises a —C(R′)₂— group, wherein one of the R′ groups is a second R′ group and the other is a third of the four. In some embodiments, such a —C(R′)₂— group is of an amino acid residue. In some embodiments, such a —C(R′)₂— group is of X⁴. In some embodiments, such a carbon atom is an alpha carbon of an amino acid residue. In some embodiments, such a carbon atom is an alpha carbon of X⁴.

In some embodiments, a methylene unit of L^{P2} is replaced with —C(R′)₂—, wherein one of the R′ groups is a second or fifth or seventh R′ group. In some embodiments, such a —C(R′)₂— group is of an amino acid residue. In some embodiments, such a —C(R′)₂— group is of X³. In some embodiments, such a carbon atom is an alpha carbon of an amino acid residue. In some embodiments, such a carbon atom is an alpha carbon of X³. In some embodiments, it is a second R′ group. In some embodiments, it is a fifth R′ group. In some embodiments, it is a seventh R′ group.

In some embodiments, a methylene unit of L^{P2} is replaced with —C(R′)₂—, wherein one of the R′ groups is a first or third R′ group. In some embodiments, such a —C(R′)₂— group is of an amino acid residue. In some embodiments, such a —C(R′)₂— group is of X⁴. In some embodiments, such a carbon atom is an alpha carbon of an amino acid residue. In some embodiments, such a carbon atom is an alpha carbon of X⁴. In some embodiments, it is a first R′ group. In some embodiments, it is a third R′ group.

### L^{AA2}

In some embodiments, L^{AA2} is or comprises amino acid residue. In some embodiments, L^{AA2} is or comprises an amino acid residue that comprises a side chain comprising an acidic or polar group. In some embodiments, L^{AA2} is an amino acid residue that comprises a side chain comprising an acidic group.

In some embodiments, L^{AA2} is L^{AR}, wherein a methylene unit is replaced with —C(R′)(R^{AS})—, wherein each variable is independently as described herein. In some embodiments, L^{A2} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA2} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA2} is —N(R′)—C(R′)(R^{AS})C(O)—, wherein each variable is independently as described herein. In some embodiments, L^{AA2} is NH—C(R′)(R^{AS})C(O)— wherein each variable is independently as described herein.

In some embodiments, L^{AS2} is L^{AS} as described herein. In some embodiments, R^{AA2} is —CO₂R, wherein R is as described herein. In some embodiments, R is H. In some embodiments, L^{AA2} is a residue of an acidic amino acid residue, e.g., Asp, Glu, etc. In some embodiments, L^{AA2} is X⁵ as described herein.

### L^{P3}

In some embodiments, L^{P3} is a covalent bond. In some embodiments, L^{P3} is an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P3} is 0-10 atoms. In some embodiments, the length of L^{P3} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P3} is or comprises an amino acid residue. In some embodiments, L^{P3} is or comprises a peptide. In some embodiments, L^{P3} is or comprises —[X]pX⁶X⁷[X]p′-, wherein each X, X⁶ and X⁷ is independently an amino acid residue, and each of p and p′ is independently 0-10. In some embodiments, L^{P3} is or comprises —X⁶X⁷—, wherein each X⁶ and X⁷ is independently an amino acid residue. In some embodiments, X⁷ is bonded to L^{AA3}. In some embodiments, a methylene unit of L^{P3} is replaced with —C(R′)₂—, wherein one of the R′ groups is the fifth, sixth, seventh or eighth R′ group. In some embodiments, X⁷ comprises —C(R′)₂—, wherein one of the R′ groups is the fifth, sixth, seventh or eighth R′ group.

### L^{AA3}

In some embodiments, L^{AA3} is or comprises amino acid residue. In some embodiments, L^{AA3} is or comprises an amino acid residue that comprises a side chain comprising an acidic or polar group. In some embodiments, L^{AA3} is an amino acid residue that comprises a side chain comprising an acidic group.

In some embodiments, L^{AA3} is L^{AR}, wherein a methylene unit is replaced with —C(R′)(R^{AS})—, wherein each variable is independently as described herein. In some embodiments, L^{AA3} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA3} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA3} is —N(R′)—C(R′)(R^{AS})C(O)—, wherein each variable is independently as described herein. In some embodiments, L^{AA3} is —NH—C(R′)(R^{AS})C(O)—, wherein each variable is independently as described herein.

In some embodiments, L^{AS3} is L^{AS} as described herein. In some embodiments, R^{AA3} is —CO₂R, wherein R is as described herein. In some embodiments, R is H. In some embodiments, L^{AA3} is a residue of an acidic amino acid residue, e.g., Asp, Glu, etc. In some embodiments, L^{AA3} is X⁶ as described herein.

In some embodiments, L^{AA3} comprises a hydrophobic group. In some embodiments, L^{AA3} is or comprises a hydrophobic amino acid residue. In some embodiments, L^{AA3} is X⁸ as described herein.

### L^{P4}

In some embodiments, L^{P4} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P4} is 0-10 atoms. In some embodiments, the length of L^{P4} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P4} is or comprises an amino acid residue. In some embodiments, L^{P4} is or comprises a peptide.

In some embodiments, L^{P4} is or comprises —[X]pX⁷X⁸[X]p′-, wherein each X, X⁷ and X⁸ is independently an amino acid residue, and each of p and p′ is independently 0-10. In some embodiments, L^{P4} is or comprises —X⁷X⁸—, wherein each X⁷ and X⁸ is independently as described herein, and X⁸ is bonded to L^{AA4}.

In some embodiments, a methylene unit of L^{P4} is replaced with —C(R′)₂—, wherein one of the R′ groups is a fifth, sixth, seventh or eighth R′ group. In some embodiments, such a —C(R′)₂— group is of an amino acid residue. In some embodiments, such a —C(R′)₂— group is of X⁷. In some embodiments, such a carbon atom is an alpha carbon of an amino acid residue. In some embodiments, such a carbon atom is an alpha carbon of X⁷. In some embodiments, it is a fifth R′ group. In some embodiments, it is a sixth R′ group. In some embodiments, it is a seventh R′ group. In some embodiments, it is an eighth R′ group.

### L^{AA4}

In some embodiments, L^{AA4} is or comprises amino acid residue. In some embodiments, L^{AA4} is or comprises an amino acid residue that comprises a side chain comprising an aromatic group.

In some embodiments, L^{AA4} is L^{AR}, wherein a methylene unit is replaced with —C(R′)(R^{AS})—, wherein each variable is independently as described herein. In some embodiments, L^{AM} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA4} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA4} is —N(R′)—C(R′)(R^{AS})C(O)—, wherein each variable is independently as described herein. In some embodiments, L^{AA4} is —NH—C(R′)(R^{AS})C(O)—, wherein each variable is independently as described herein.

In some embodiments, L^{AS4} is L^{AS} as described herein. In some embodiments, R^{AA4} is optionally substituted C₆₋₁₄ aryl. In some embodiments, R^{AA4} is optionally substituted phenyl. In some embodiments, R^{AA4} is phenyl. In some embodiments, R^{AA4} is optionally substituted 6-membered monocyclic heteroaryl having 1-4 heteroatoms. In some embodiments, R^{AA4} is optionally substituted 9-membered bicyclic heteroaryl having 1-4 heteroatoms. In some embodiments, R^{AA4} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA4} is optionally substituted S

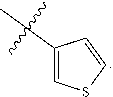 [SMILES: *c1ccsc1]

In some embodiments, R^{AA4} is optionally substituted

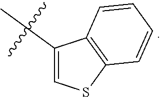 [SMILES: *c1csc2ccccc12]

In some embodiments, R^{AA4} is optionally substituted

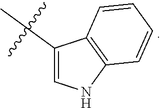 [SMILES: *c1c[nH]c2ccccc12]

In some embodiments, L^{AA4} is an aromatic amino acid residue as described herein. In some embodiments, L^{AA4} is X⁹ as described herein.

### L^{P5}

In some embodiments, L^{P5} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L⁵ is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P5} is or comprises an amino acid residue. In some embodiments, L^{P5} is or comprises a peptide.

In some embodiments, L^{P5} is or comprises —[X]pX¹¹[X]p′-, wherein each variable is independently as described herein. In some embodiments, L^{P5} is or comprises —X¹⁰X¹¹—, wherein each X¹⁰ and X¹¹ is independently as described herein, and X¹¹ is bonded to L^{AA5}.

In some embodiments, L^{P5} comprises a —C(R′)₂— group, wherein one of the R′ groups is a fourth R′ group. In some embodiments, L^{P5} comprises a —C(R′)₂— group, wherein one of the R′ groups is a second R′ group. In some embodiments, such a —C(R′)₂— group is of an amino acid residue. In some embodiments, such a —C(R′)₂— group is of X¹¹. In some embodiments, such a carbon atom is an alpha carbon of an amino acid residue. In some embodiments, such a carbon atom is an alpha carbon of X¹¹.

In some embodiments, L^{P5} comprises a —C(R′)₂— group, wherein one of the R′ groups is a fifth, sixth, seventh or eighth R′ group. In some embodiments, such a —C(R′)₂— group is of an amino acid residue. In some embodiments, such a —C(R′)₂— group is of X₁₀. In some embodiments, such a carbon atom is an alpha carbon of an amino acid residue. In some embodiments, such a carbon atom is an alpha carbon of X⁰. In some embodiments, it is a fifth R′ group. In some embodiments, it is a sixth R′ group. In some embodiments, it is a seventh R′ group. In some embodiments, it is an eighth R′ group.

### L^{AA5}

In some embodiments, L^{AA5} is or comprises amino acid residue. In some embodiments, L^{AA5} is or comprises an amino acid residue that comprises a side chain comprising an aromatic group.

In some embodiments, L^{AA5} is L^{AR}, wherein a methylene unit is replaced with —C(R′)(R^{AS})—, wherein each variable is independently as described herein. In some embodiments, L^{AA5} is an optionally substituted, bivalent C₁-C₆ (e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA5} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA5} is —N(R′)—C(R′)(R^{AS})C(O)—, wherein each variable is independently as described herein. In some embodiments, L^{AA5} is —NH—C(R′)(R^{AS})C(O)—, wherein each variable is independently as described herein.

In some embodiments, L^{AS5} is L^{AS} as described herein. In some embodiments, R^{AA5} is optionally substituted C₆₋₁₄ aryl. In some embodiments, R^{AA5} is optionally substituted phenyl. In some embodiments, R^{AA5} is phenyl. In some embodiments, R^{AA5} is optionally substituted 10-membered C₁₀ bicyclic aryl. In some embodiments, R^{AA5} is optionally substituted 5-membered monocyclic heteroaryl having 1-4 heteroatoms. In some embodiments, R^{AA5} is optionally substituted 6-membered monocyclic heteroaryl having 1-4 heteroatoms. In some embodiments, R^{AA5} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA5} is optionally substituted

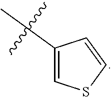 [SMILES: *c1ccsc1]

In some embodiments, R^{AA5} is optionally substituted

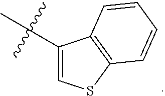 [SMILES: *c1csc2ccccc12]

In some embodiments, R^{AA5} is optionally substituted

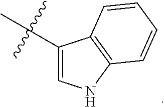 [SMILES: *c1c[nH]c2ccccc12]

In some embodiments, L^{AA5} is an aromatic amino acid residue as described herein. In some embodiments, L^{AA5} is X¹² as described herein.

### L^{P6}

In some embodiments, L^{P6} is a covalent bond. In some embodiments, L^{P6} is an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the length of L^{P6} is 0-10 atoms (e.g., 0, 1, 2, 3, 4, 5, 6, 7, 8, 9, or 10, etc.). In some embodiments, the length of L^{P6} is 2-10 atoms. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms. In some embodiments, one or more methylene units are independently replaced with —N(R′)—, —C(R′)₂—, —C(O)— or —C(O)N(R′)—. In some embodiments, a methylene unit is replace with —N(R′)—. In some embodiments, a methylene unit is replace with —C(R′)₂—. In some embodiments, a methylene unit is replace with —C(O)—. In some embodiments, a methylene unit is replace with —C(O)N(R′)—. In some embodiments, each methylene unit is independently replaced with —N(R′)—, —C(R′)₂— or —C(O)—. In some embodiments, L^{P6} is or comprises an amino acid residue. In some embodiments, L^{P6} is or comprises a peptide.

### L^{AA6}

In some embodiments, L^{AA6} is or comprises amino acid residue. In some embodiments, L^{AA6} is or comprises an amino acid residue that comprises a side chain comprising an aromatic group.

In some embodiments, L^{AA6} is L^{AR}, wherein a methylene unit is replaced with —C(R′)(R^{AS})—, wherein each variable is independently as described herein. In some embodiments, L^{AA6} is optionally substituted, bivalent C₁-C₆(e.g., C₁, C₂, C₃, C₄, C₅, or C₆) aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —X(O)₂—, —S(O)₂N(R′)—, —C(O)S—, or —C(O)O—, wherein each variable is independently as described herein. In some embodiments, L^{AA6} is an optionally substituted bivalent C₂-C₄ aliphatic group, herein one or more methylene units of the group are optionally and independently replaced with —C(R′)₂—, —C(R′)(R^{AS})—, -Cy-, —O—, —S—, —S—S—, —N(R′)—C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein each variable is independently as described herein. In some embodiments, L^{AA6} is —NH—C(R′)(R^{AS})—C(O)—, wherein each variable is independently as described herein.

In some embodiments, L^{AS6} is L^{AS} as described herein. In some embodiments, R^{AA6} is optionally substituted C₆₋₁₄ aryl. In some embodiments, R^{AA6} is optionally substituted phenyl. In some embodiments, R^{AA6} is phenyl. In some embodiments, R^{AA6} is optionally substituted 10-membered C₁₀ bicyclic aryl. In some embodiments, R^{AA6} is optionally substituted 5-membered monocyclic heteroaryl having 1-4 heteroatoms. In some embodiments, R^{AA6} is optionally substituted 6-membered monocyclic heteroaryl having 1-4 heteroatoms. In some embodiments, R^{AA6} is optionally substituted 10-membered bicyclic heteroaryl having 1-4 heteroatoms. In some embodiments, a heteroaryl has no more than one heteroatom. In some embodiments, a heteroaryl has two or more heteroatoms. In some embodiments, a heteroatom is oxygen. In some embodiments, a heteroatom is nitrogen. In some embodiments, a heteroatom is sulfur. In some embodiments, R^{AA6} is optionally substituted

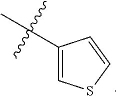 [SMILES: *c1ccsc1]

In some embodiments, R^{AA6} is optionally substituted

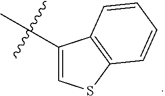 [SMILES: *c1csc2ccccc12]

In some embodiments, R^{AA6} is optionally substituted

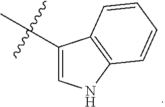 [SMILES: *c1c[nH]c2ccccc12]

In some embodiments, L^{AA6} is an aromatic amino acid residue as described herein. In some embodiments, L^{AA6} is X¹³ as described herein.

### L^{P7}

In some embodiments, L^{P7} is a covalent bond. In some embodiments, L^{P7} is an optionally substituted, bivalent C₁-C₂₅ (e.g., C₁₋₂₀, C₁₋₁₅, C₁₋₁₀, C₁₋₅, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₂₅ (e.g., C₁₋₂₀, C₁₋₁₅, C₁₋₁₀, C₁₋₅, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₂₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₁₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{P7} is an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, is or comprises —X¹⁴—[X]p′-, wherein p′ is 0-10. In some embodiments, X¹⁴ is bonded to L^{AA6}. In some embodiments, L^{P7} comprises a —C(R′)₂— group, wherein one of the R′ groups is a sixth or eighth R′ group. In some embodiments, such a —C(R′)₂— group is of an amino acid residue. In some embodiments, such a —C(R′)₂— group is of X¹⁴. In some embodiments, such a carbon atom is an alpha carbon of an amino acid residue. In some embodiments, such a carbon atom is an alpha carbon of X¹⁴. In some embodiments, it is a sixth R′ group. In some embodiments, it is an eighth R′ group.

### L^{AS}

In some embodiments, L^{AS} is a covalent bond. In some embodiments, L^{AS} is an optionally substituted, bivalent C₁-C₁₀ (e.g., C₁₋₅, C₁, C₂, C₃, C₄, C₅, C₆, C₇, C₈, C₉, or C₁₀) aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{AS} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—. In some embodiments, L^{AS} is an optionally substituted, bivalent C₁-C₁₀ aliphatic group, wherein one or more methylene units of the group are optionally and independently replaced with —O—, —S—, or —N(R′)—. In some embodiments, L^{AS} is an optionally substituted, bivalent C₁-C₁₀ alkylene group. In some embodiments, L^{AS} is optionally substituted —CH₂—. In some embodiments, L^{AS} is —CH₂—. In some embodiments, the length of L^{AS} is 1, 2, 3, 4, 5, 6, 7, 8, 9, or 10 atoms. In some embodiments, it is 1 atom. In some embodiments, it is 2 atoms. In some embodiments, it is 3 atoms. In some embodiments, it is 4 atoms. In some embodiments, it is 5 atoms. In some embodiments, it is 6 atoms. In some embodiments, it is 7 atoms. In some embodiments, it is 8 atoms. In some embodiments, it is 9 atoms. In some embodiments, it is 10 atoms.

In some embodiments, an agent of formula I is a stapled peptide as described herein. In some embodiments, an agent of formula I is an agent selected from Table E2 or a pharmaceutically acceptable salt thereof. In some embodiments, an agent of formula I is an agent selected from Table E3 or a pharmaceutically acceptable salt thereof.

Among other things, the present disclosure provides agents, e.g. peptides, that can bind to beta-catenin. In some embodiments, an agent is or comprises X¹X²X³X⁴X⁵X⁶X⁷X⁸X⁹X¹⁰X¹¹X¹²X¹³X¹⁴ wherein each of X¹, X², X³, X⁴, X⁵, X⁶, X⁷, X⁸, X⁹, X¹⁰, X¹¹, X¹², X¹³ and X¹⁴ is independently an amino acid residue. In some embodiments, an agent is or comprises [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and each of X, X⁰, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

Various amino acid residues, e.g., those of formula A-I, A-II, A-III, A-IV, A-V, A-VI, PA, etc., can be utilized in accordance with the present disclosure. Certain useful amino acid residues are described in the present disclosure.

In some embodiments, each of X² and X⁵ is independently an acidic residue as described herein. In some embodiments, each of X², X⁵ and X⁶ is independently an acidic residue as described herein. In some embodiments, each of X⁹, X¹² and X¹³ are independently an amino acid residue comprising a side chain that comprises an aromatic group.

In some embodiments, X² is an acidic residue. In some embodiments, X² comprises a side chain that comprises —COOH or a derivative thereof. In some embodiments, X² comprises a side chain that comprises —COOH. In some embodiments, X² is Asp. Various other amino acid residues for X² are described else in the present disclosure.

In some embodiments, X⁵ is an acidic residue. In some embodiments, X⁵ comprises a side chain that comprises —COOH or a derivative thereof. In some embodiments, X⁵ comprises a side chain that comprises —COOH. In some embodiments, X⁵ is Asp. Various other amino acid residues for X⁵ are described else in the present disclosure.

In some embodiments, X⁶ is an acidic residue. In some embodiments, X⁶ comprises a side chain that comprises —COOH or a derivative thereof. In some embodiments, X⁶ comprises a side chain that comprises —COOH. In some embodiments, X⁶ is Asp. Various other amino acid residues for X⁶ are described else in the present disclosure.

In some embodiments, X⁹ comprises a side chain that comprises an aromatic group. In some embodiments, X⁹ comprises a side chain that comprises —R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X⁹ is Phe. Various other amino acid residues for X⁹ are described else in the present disclosure.

In some embodiments, X² comprises a side chain that comprises an aromatic group. In some embodiments, X¹² comprises a side chain that comprises —R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X² is 3Thi. In some embodiments, X¹² is 2F3MeF. In some embodiments, X¹² is Phe. Various other amino acid residues for X¹² are described else in the present disclosure.

In some embodiments, X¹³ comprises a side chain that comprises an aromatic group. In some embodiments, X¹³ comprises a side chain that comprises —R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X¹³ is BtzA. In some embodiments, X¹³ is 34ClF. In some embodiments, X¹³ is 2NapA. Various other amino acid residues for X¹³ are described else in the present disclosure.

As described herein, in some embodiments, a peptide is a stapled peptide. In some embodiments, an agent is or comprises a peptide, wherein a peptide is a stapled peptide. In some embodiments, a peptide is a stitched peptide. In some embodiments, a peptide comprises three or more staples as described herein. In some embodiments, a peptide comprises three or more staples within a region having a length of, e.g., 11-15, such as 11, 14, etc., amino acid residues as described herein. In some embodiments, such a peptide provides improved rigidity, activity, delivery, solubility, and/or other desired properties comprising a reference peptide that is not stapled or that comprises fewer staples.

In some embodiments, the present disclosure provides an agent, e.g., a peptide, comprising X¹X²X³X⁴X⁵X⁶X⁷X⁸X⁹X¹⁰X¹¹X¹²X¹³X¹⁴, wherein X¹, X², X³, X⁴, X⁵, X⁶, X⁷, X⁸, X⁹, X¹⁰, X¹¹, X¹², X¹³, and X¹⁴ are each independently an amino acid residue and comprises two or more pairs of amino acid residues, wherein each pair of amino acid residues are independently two amino acid residues suitable for stapling or stapled. In some embodiments, the present disclosure provides an agent, e.g., a peptide, comprising X¹X²X³X⁴X⁵X⁶X⁷X⁸X⁹X¹⁰X¹¹X¹²X¹³X¹⁴, wherein X¹, X², X³, X⁴, X⁵, X⁶, X⁷, X⁸, X⁹, X¹⁰, X¹¹, X¹², X¹³, and X¹⁴ are each independently an amino acid residue and comprises two or more pairs of amino acid residues, wherein each pair of amino acid residues are independently three amino acid residues suitable for stapling or stapled.

In some embodiments, the present disclosure provides an agent, e.g., a peptide, comprising [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ are each independently an amino acid residue and comprises two or more pairs of amino acid residues, wherein each pair of amino acid residues are independently two amino acid residues suitable for stapling or stapled. In some embodiments, the present disclosure provides an agent, e.g., a peptide, comprising [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ are each independently an amino acid residue and comprises three or more pairs of amino acid residues, wherein each pair of amino acid residues are independently two amino acid residues suitable for stapling or stapled. In some embodiments, each amino acid residue in such pairs of amino acid residues are independently selected from X¹, X², X³, X⁴, X⁵, X⁶, X⁷, X⁸, X⁹, X¹⁰, X¹¹, X¹², X¹³, and X¹⁴.

In some embodiments, there are three such pairs of amino acid residues. In some embodiments, there are four such pairs of amino acid residues. In some embodiments, there are four or more such pairs of amino acid residues. In some embodiments, each pair is independently not stapled. In some embodiments, one or more pairs are independently stapled. In some embodiments, two or more pairs are independently stapled. In some embodiments, three or more pairs are independently stapled. In some embodiments, four or more pairs are independently stapled. In some embodiments, two pairs are independently stapled. In some embodiments, three pairs are independently stapled. In some embodiments, four pairs are independently stapled.

In some embodiments, a pair is X¹ and X⁴. In some embodiments, a pair is X⁴ and X¹¹. In some embodiments, a pair is X¹ and X³. In some embodiments, a pair is X⁴ and X¹¹. In some embodiments, a pair is X¹⁰ and X¹⁴. In some embodiments, a pair is X⁷ and X¹⁰. In some embodiments, a pair is X⁷ and X¹⁴. In some embodiments, a pair is X³ and X⁷.

In some embodiments, a pair is X¹ and X¹⁴ and a pair is X⁴ and X¹¹. In some embodiments, a pair is X¹ and X¹⁴, a pair is X⁴ and X¹¹ and a pair is X¹⁰ and X¹⁴. In some embodiments, a pair is X¹ and X¹⁴, a pair is X⁴ and X¹ and a pair is X⁷ and X¹⁰. In some embodiments, a pair is X¹ and X¹⁴, a pair is X⁴ and X¹¹ and a pair is X⁷ and X¹⁴. In some embodiments, a pair is X¹ and X¹⁴, a pair is X⁴ and X¹¹, a pair is X³ and X⁷, and a pair is X⁷ and X¹⁴. In some embodiments, each pair is independently a pair of amino acid residues suitable for stapling. In some embodiments, each pair is independently stapled.

In some embodiments, a pair is X¹ and X³, a pair is X⁴ and X¹¹, and a pair is X¹⁰ and X¹⁴. In some embodiments, each pair is independently a pair of amino acid residues suitable for stapling. In some embodiments, each pair is independently stapled.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
   each of X⁰, X¹, X², X³, X⁴, X⁵, X⁶, X⁷, X₈, X⁹, X¹⁰, X¹¹, X¹², X¹³, X¹⁴, X¹⁵, X¹⁶, and X¹⁷ is independently an amino acid residue, wherein the agent binds to beta-catenin.

In some embodiments, X² comprises a side chain comprising an acidic or a polar group. In some embodiments, X² comprises a side chain comprising an acidic group. In some embodiments, X² comprises a side chain comprising a polar group. In some embodiments, X⁵ comprises a side chain comprising an acidic or a polar group. In some embodiments, X⁵ comprises a side chain comprising an acidic group. In some embodiments, X⁵ comprises a side chain comprising a polar group. In some embodiments, X¹³ comprises a side chain comprising an optionally substituted aromatic group. In some embodiments, two or more of X¹, X³, X⁴, X⁷, X¹⁰, X¹¹ and X¹⁴ are each independently an amino acid residue suitable for stapling, or are each independently stapled.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled. In some embodiments, three or more of X¹, X³, X⁴, X⁷, X¹⁰, X¹¹ and X¹⁴ are each independently an amino acid residue suitable for stapling, or are each independently stapled. In some embodiments, four or more of X¹, X³, X⁴, X⁷, X¹⁰, X¹¹ and X¹⁴ are each independently an amino acid residue suitable for stapling, or are each independently stapled. In some embodiments, five of X¹, X³, X⁴, X⁷, X¹⁰, X¹¹ and X¹⁴ are each independently an amino acid residue suitable for stapling, or are each independently stapled. In some embodiments, X¹ and X⁴ are each independently an amino acid residue suitable for stapling. In some embodiments, X¹ and X³ are each independently an amino acid residue suitable for stapling. In some embodiments, X⁴ and X¹¹ are each independently an amino acid suitable for stapling. In some embodiments, X¹, X⁴, and X¹¹ are each independently an amino acid residue suitable for stapling. In some embodiments, X¹⁰ and X¹⁴ are each independently an amino acid residue suitable for stapling. In some embodiments, X⁷ and X¹⁰ are each independently an amino acid residue suitable for stapling. In some embodiments, X⁷ and X¹⁴ are each independently an amino acid residue suitable for stapling. In some embodiments, X³ and X⁷ are each independently an amino acid residue suitable for stapling. In some embodiments, X¹ and X⁴ are connected by a staple. In some embodiments, X¹ and X³ are connected by a staple. In some embodiments, X⁴ and X¹¹ are connected by a staple. In some embodiments, X¹ and X⁴ connected by a staple, and X⁴ and X¹¹ are connected by a staple. In some embodiments, X¹⁰ and X¹⁴ are connected by a staple. In some embodiments, X⁷ and X¹⁰ are connected by a staple. In some embodiments, X⁷ and X¹⁴ are connected by a staple. In some embodiments, X³ and X⁷ are connected by a staple.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X¹⁰ and X¹⁴ are connected by a staple.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X⁷ and X¹⁰ are connected by a staple.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X⁷ and X¹⁴ are connected by a staple.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple; and X¹⁰ and X¹⁴ are connected by a staple and/or X³ and X⁷ are connected by a staple.

In some embodiments, the present disclosure provides an agent, which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X³ are connected by a staple, X⁴ and X¹¹ are connected by a staple; and X¹⁰ and X¹⁴ are connected by a staple.

In some embodiments, X² comprises a side chain comprising an acidic (e.g., —COOH) or a polar group. In some embodiments, X² comprises a side chain comprising an acid group. In some embodiments, X⁵ comprises a side chain comprising an acidic or a polar group. In some embodiments, X⁵ comprises a side chain comprising an acid group. In some embodiments, X⁶ comprises a side chain comprising an acidic or a polar group. In some embodiments, X⁶ comprises a side chain comprising an acid group. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X² and X⁵ each independently comprise a side chain comprising an acidic or a polar group. In some embodiments, X² and X⁶ each independently comprise a side chain comprising an acidic or a polar group. In some embodiments, X⁵ and X⁶ each independently comprise a side chain comprising an acidic or a polar group. In some embodiments, X² and X⁵ each independently comprise a side chain comprising an acidic group. In some embodiments, X² and X⁶ each independently comprise a side chain comprising an acidic group. In some embodiments, X⁵ and X⁶ each independently comprise a side chain comprising an acidic group. In some embodiments, X², X⁵ and X⁶ each independently comprise a side chain comprising an acidic or a polar group. In some embodiments, X², X⁵ and X⁶ each independently comprise a side chain comprising an acidic group. In some embodiments, each of X⁹ and X¹² independently comprises a side chain comprising an optionally substituted aromatic group. In some embodiments, each of X⁹ and X¹³ independently comprises a side chain comprising an optionally substituted aromatic group. In some embodiments, each of X⁹, X¹² and X¹³ independently comprises a side chain comprising an optionally substituted aromatic group. In some embodiments, each of X² and X⁵ independently comprises a side chain comprising an acidic group (e.g., —COOH), and each of X⁹, X¹² and X¹³ independently comprises a side chain comprising an optionally substituted aromatic group. In some embodiments, each of X², X⁵ and X⁶ independently comprises a side chain comprising an acidic group (e.g., —COOH), and each of X⁹, X¹² and X¹³ independently comprises a side chain comprising an optionally substituted aromatic group.

As described herein, various types of amino acid residues (e.g., those of amino acids having the structure of formula A-I, A-II, A-III, A-IV, A-V, A-VI, etc.) can be utilized in accordance with the present disclosure. Certain examples are described herein for X⁰, X¹, X², X³, X⁴, X⁵, X⁶, X⁷, X⁸, X⁹, X¹⁰, X¹¹, X¹, X¹³, X¹⁴, X¹⁵, X¹⁶, X¹⁷, etc.

In some embodiments, p0 is 0. In some embodiments, p0 is 1. Various types of amino acid residues can be used for X⁰. In some embodiments, X⁰ is selected from Gly, Sar, and NMebAla. In some embodiments, X⁰ is Gly. In some embodiments, X⁰ is Sar. In some embodiments, X⁰ is NMebAla. In some embodiments, X⁰ is present in various peptides (e.g., in some embodiments, p0 is 1). In some embodiments, X⁰ is absent from various peptides (e.g., in some embodiments, p0 is 0).

In some embodiments, X⁰ is a N-terminus residue. In some embodiments, it is bonded to a N-terminal group.

In some embodiments, X⁰ is an amino acid reside suitable for stapling.

In some embodiments, an amino acid residue suitable for stapling comprises a double bond, e.g., a terminal double bond in its side chain. In some embodiments, it has a side chain having the structure of -L^{a}-CH═CH₂. In some embodiments, it is a residue of an amino acid having the structure of formula A-II or A-III or a salt thereof. In some embodiments, X⁰ is —N(R^{a1})-L^{a1}-C(-L^{a}-CH═CH₂)(R^{a3})-L^{a2}-C(O)—, wherein each variable is independently as described herein. In some embodiments, X⁰ is —N(R^{a1})—C(-L^{a}-CH═CH₂)(R^{a3})—C(O)—, wherein each variable is independently as described herein. In some embodiments, X⁰ is a residue of PL3 and stapled.

In some embodiments, X⁰ is N(-L^{a}-CH═CH₂)(R^{a1})-L^{a1}-C(-L^{a}-CH═CH₂)(R^{a3})-L^{a2}-C(O)—, or —N(-L^{a}-CH═CH₂)-L^{a1}-C(-L^{a}-CH═CH₂)(R^{a3})-L^{a2}-C(O)—, wherein each variable is independently as described herein. In some embodiments, X⁰ is —N(-L^{a}-CH═CH₂)—C(-L^{a}-CH═CH₂)(R^{a3})—C(O)—, wherein each variable is independently as described herein.

In some embodiments, X⁰ is S5. In some embodiments, X⁰ is S6.

In some embodiments, X⁰ is stapled. Various types of staples may be utilized as described herein. In some embodiments, X⁰ is stapled with X⁴. In some embodiments, X⁴ is stapled with X¹¹. In some embodiments, a stapled peptide comprises X⁰—X⁴—X¹¹ stapling. In some embodiments, a stapled peptide comprises another staple, e.g., X¹⁰—X¹⁴.

In some embodiments, X⁰ is X¹ as described herein.

Various types of amino acid residues can be used for X¹, e.g., a residue of an amino acid of formula A-I, A-II, A-III, A-IV, A-V, A-VI, etc. or a salt thereof in accordance with the present disclosure. In some embodiments, X¹ is —N(R^{a1})-L^{a1}-C(R^{a2})(R^{a3})-L^{a2}-C(O)—, wherein each variable is independently as described herein. In some embodiments, X¹ is —N(R^{a1})—C(R^{a2})(R^{a3})—C(O)—, wherein each variable is independently as described herein. In some embodiments, X¹ is —N(R^{a1})—C(R^{a2})H—C(O)—, wherein each variable is independently as described herein. In some embodiments, R^{a1} is —H. In some embodiments, R^{a3} is —H.

As shown herein (e.g., for various amino acids and residues thereof), in various embodiments, L^{a} is L as described herein. For example, in some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, one or more methylene units of L are independently replaced with —C(O)—, —N(R′)—, -Cy- or —O—. In some embodiments, a methylene unit is replaced with —C(O)—. In some embodiments, a methylene unit is replaced with —N(R′)—. In some embodiments, a methylene unit is replaced with -Cy-. In some embodiments, -Cy- is optionally substituted phenylene. In some embodiments, -Cy- is 1,2-phenylene. In some embodiments, a methylene unit is replaced with —O—. In some embodiments, L is —C(O)—(CH₂)n-. In some embodiments, L is —C(O)—(CH₂)₂—. In some embodiments, L is —C(O)—(CH₂)₃—. In some embodiments, L is —C(O)-1,2-phenylene-O—CH₂—. As appreciated by those skilled in the art, embodiments described for each group or moiety, e.g., L, is applicable to all groups that can be such a group or moiety (e.g., L^{a}, L^{s1}, L^{s2}, L^{s3}, etc.), no matter where such embodiments are described.

In some embodiments, X¹ is a residue of amino acid that comprises an optionally substituted ring. In some embodiments, the amino group of X¹ is part of an optionally substituted ring. In some embodiments, X¹ is an amino acid as described herein, e.g., of formula A-I, A-II, A-III, etc. In some embodiments, R^{a1} and R^{a3} are taken together to form an optionally substituted ring, e.g., an optionally substituted 3-10 membered ring. In some embodiments, R^{a1} and R^{a3} are taken together with their intervening atoms to form an optionally substituted 3-10 membered saturated or partially saturated ring having, in addition to the intervening atoms, 0-5 heteroatoms. In some embodiments, a formed ring is saturated. In some embodiments, a formed ring is monocyclic. In some embodiments, a formed ring has no heteroatoms in addition to the intervening atoms. In some embodiments, L^{a1} and L^{a2} are covalent bonds. In some embodiments, a formed ring is unsubstituted. In some embodiments, a formed ring is substituted. In some embodiments, a substituent comprises a double bond which is suitable for metathesis with another double bond to form a staple. In some embodiments, X¹ is MePro.

In some embodiments, X¹ is an amino acid reside suitable for stapling.

In some embodiments, an amino acid residue suitable for stapling comprises a double bond, e.g., a terminal double bond in its side chain. In some embodiments, it has a side chain having the structure of -L^{a}-CH═CH₂. In some embodiments, it is a residue of an amino acid having the structure of formula A-II or A-III or a salt thereof. In some embodiments, X¹ is —N(R^{a1})-L^{a1}-C(-L^{a}-CH═CH₂)(R^{a3})-L^{a2}-C(O)—, wherein each variable is independently as described herein. In some embodiments, X¹ is —N(R^{a1})—C(-L^{a}-CH═CH₂)(R^{a3})—C(O)—, wherein each variable is independently as described herein. In some embodiments, X¹ is a residue of PL3 and stapled.

In some embodiments, X¹ is N(-L^{a}-CH═CH₂)(R^{a1})-L^{a1}-C(-L^{a}-CH═CH₂)(R^{a3})-L^{a2}-C(O)—, or —N(-L^{a}-CH═CH₂)-L^{a1}-C(-L^{a}-CH═CH₂)(R^{a3})-L^{a2}-C(O)—, wherein each variable is independently as described herein. In some embodiments, X¹ is —N(-L^{a}-CH═CH₂)—C(-L^{a}-CH═CH₂)(R^{a3})—C(O)—, wherein each variable is independently as described herein.

In some embodiments, it is PL3. In some embodiments, it is an residue of [4pentenyl]MePro (

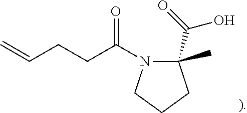

some embodiments, it is a residue of [5hexenyl]MePro (

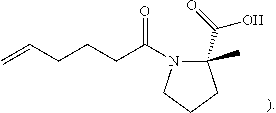 [SMILES: C=CCCCC(=O)N1CCC[C@@]1(C)C(=O)O]

In some embodiments, it is an residue of [BzAm20Allyl]MePro (

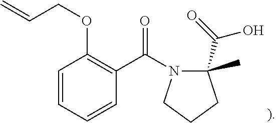 [SMILES: C=CCOc1ccccc1C(=O)N1CCC[C@@]1(C)C(=O)O]

In some embodiments, X¹ is PL3. In some embodiments, X¹ is S5. In some embodiments, X¹ is MePro. In some embodiments, X¹ is Asp. In some embodiments, X¹ is S6. In some embodiments, X¹ is Pro. In some embodiments, X¹ is Ala. In some embodiments, X¹ is Ser. In some embodiments, X¹ is ThioPro. In some embodiments, X¹ is Gly. In some embodiments, X¹ is NMebAla. In some embodiments, X¹ is Asn. In some embodiments, X¹ is TfeGA. In some embodiments, X¹ is Glu. In some embodiments, X¹ is an acidic amino acid residue. In some embodiments, X¹ is a polar amino acid residue. In some embodiments, X¹ comprises a hydrophobic side chain.

In some embodiments, an agent comprises a N-terminal group. In some embodiments, X¹ is bonded to a N-terminal group. In some embodiments, X¹ comprises a N-terminal group. In some embodiments, a N-terminal group is Ac, 4pentenyl, 5hexenyl, BzAm20Allyl, Hex, Bua, 2PyzCO, 3Phc3, MeOPr, lithocholate, 2FPhc, PhC, MeSO2, Ts, Isobutyryl, Isovaleryl, EtHNCO, TzPyr, 15PyraPy, 8IAP, 3PydCO, 2PyBu, 2PymCO, 5PymCO, or 4PymCO. In some embodiments, a N-terminal group is Ac, 2PyBu, 1Imidac, 2F2PyAc, 2IAPAc, 124TriPr, 6QuiAc, 3PyAc, 123TriAc, 1PyrazoleAc, 4PyPrpc, 3PyPrpc, 5PymAc, 1PydoneAc, 124TriAc, 3IAPAc, Me2NAc, 4MePipzPrpC, MePipAc, MeImid4SO2, 8QuiSO2, mPEG4, mPEG8, mPEG16, mPEG24, NPyroR3, C3a, Bua, isobutyryl, Cpc, Bnc, CF3CO, 2PyCypCO, Cbc, CypCO, 4THPCO, 2PyzCO, 3Phc3, MeOPr, lithocholate, 2FPhc, PhC, MeSO2, Ts, Isovaleryl, EtHNCO, 5hexenyl, TzPyr, 15PyraPy, 8IAP, 3PydCO, 2PymCO, 5PymCO, 4PymCO, or 4pentenyl. In some embodiments, a N-terminal group contains a moiety, e.g., a terminal olefin, for stapling. In some embodiments, a N-terminal group is Ac. In some embodiments, a N-terminal group is NPyroR3. In some embodiments, a N-terminal group is 5hexenyl. In some embodiments, a N-terminal group is 4pentenyl.

In some embodiments, X¹ is Ac-PL3, Ac-S5, NPyroR3-Asp, Ac-MePro, 5hexenyl-MePro, Ac-S6, 4pentenyl-MePro, Ac-Pro, Ac-Ala, Bua-PL3, C3a-PL3, Cpc-PL3, Cbc-PL3, CypCO-PL3, 4THPCO-PL3, Isobutyryl-PL3, Ac-Asp, Ac-Ser, Ts-PL3, 15PyraPy-PL3, 2PyBu-PL3, 4PymCO-PL3, 4pentenyl-ThioPro, 4PyPrpc-PL3, 3IAPAc-PL3, 4MePipzPrpC-PL3, MePipAc-PL3, MeImid4SO2-PL3, BzAm20Allyl-MePro, Ac-Gly, Ac-Sar, Ac-NMebAla, Hex-PL3, 2PyzCO-PL3, 3Phc3-PL3, MeOPr-PL3, lithocholate-PL3, 2FPhc-PL3, PhC-PL3, MeSO2-PL3, Isovaleryl-PL3, EtHNCO-PL3, TzPyr-PL3, 8IAP-PL3, 3PydCO-PL3, 2PymCO-PL3, 5PymCO-PL3, 1Imidac-PL3, 2F2PyAc-PL3, 2IAPAc-PL3, 124TriPr-PL3, 6QuiAc-PL3, 3PyAc-PL3, 123TriAc-PL3, 1PyrazoleAc-PL3, 3PyPrpc-PL3, 5PymAc-PL3, 1PydoneAc-PL3, 124TriAc-PL3, Me2NAc-PL3, 8QuiSO2-PL3, mPEG4-PL3, mPEG8-PL3, mPEG16-PL3, mPEG24-PL3, NPyroR3-Asn, or NPyroR3-Ser. In some embodiments, X¹ is Ac-PL3. In some embodiments, X¹ is Ac-S5. In some embodiments, X¹ is NPyroR3-Asp. In some embodiments, X¹ is Ac-MePro. In some embodiments, X¹ is Ac-S6. In some embodiments, X¹ is 4pentenyl-MePro. In some embodiments, X¹ is Ac-Pro. In some embodiments, X¹ is Ac-Ala. In some embodiments, X¹ is Bua-PL3. In some embodiments, X¹ is C₃a-PL3. In some embodiments, X¹ is Cpc-PL3. In some embodiments, X¹ is Cbc-PL3. In some embodiments, X¹ is CypCO-PL3. In some embodiments, X¹ is 4THPCO-PL3. In some embodiments, X¹ is Isobutyryl-PL3. In some embodiments, X¹ is Bnc-PL3. In some embodiments, X¹ is CF3CO-PL3.

In some embodiments, X¹ is or comprises a residue of an amino acid or a moiety selected from Table A-I, Table A-II, Table A-III and Table A-IV.

In some embodiments, X¹ is stapled (a staple bonds to X¹). In some embodiments, X¹ is a residue of PL3 and stapled. In some embodiments, X¹ is stapled with X⁴. In some embodiments, a staple connecting a pair of amino acid residues, e.g., X¹ and X⁴, has the structure of L^{s}, -L^{s1}-L^{s2}-L^{s3}-, wherein L^{s1} is L^{a} of one amino acid residue, e.g., X¹, and L^{s3} is L^{a} of the other amino acid residue, e.g., X⁴.

As described herein, in some embodiments, a staple is L^{s}. In some embodiments, L^{s1} is L^{a} of one amino acid residue of a pair of stapled amino acid residues, and L^{s3} is L^{a} of the other amino acid residue of a pair of stapled amino acid residues. In some embodiments, L^{s} is -L^{a}-L^{s2}-L^{a}-, wherein each variable is independently as described herein. Various embodiments of L^{a} are described herein. In some embodiments, L^{s1} is an optionally substituted bivalent linear or branched C₁₋₁₀ hydrocarbon chain. In some embodiments, L^{s3} is an optionally substituted bivalent linear or branched C₁₋₁₀ hydrocarbon chain. In some embodiments, each of L^{s1} and L^{s3} is independently an optionally substituted bivalent linear or branched C₁₋₁₀ hydrocarbon chain. In some embodiments, each of L^{s1} and L^{s3} is independently —(CH₂)n-, wherein n is 1-10. In some embodiments, L^{s1} is —CH₂—. In some embodiments, L^{s3} is —(CH₂)₃—.

In some embodiments, L^{s2} is L as described herein. In some embodiments, L is optionally substituted —CH═CH—. In some embodiments, L is optionally substituted —CH₂—CH₂—. In some embodiments, L is —CH₂—CH₂—.

In some embodiments, L^{s} is —CH₂—CH═CH—(CH₂)₃—. In some embodiments, L^{s} is —(CH₂)₆—. In some embodiments, such a staple connects X¹ and X⁴. In some embodiments, such a staple may connect other pairs of stapled amino acid residues.

In some embodiments, a staple, e.g., L^{s}, is bonded to two backbone atoms. In some embodiments, it is bonded to two carbon backbone atoms. In some embodiments, it is independently bonded to an alpha carbon atom of an amino acid residue at each end. In some embodiments, it is bonded to a nitrogen backbone atom (e.g., of an alpha-amino group) and a carbon backbone atom (e.g., an alpha-carbon atom). In some embodiments, it is bonded to two nitrogen backbone atoms (e.g., in some embodiments, each independently of an alpha-amino group).

In some embodiments, X¹ is [4pentyenyl]MePro, [5pentenyl]MePro or [BzAm20Allyl]MePro. In some embodiments, X¹ is stapled with X³. In some embodiments, a staple connecting X¹ and X³ has the structure of L^{s} as described herein.

As described herein, in some embodiments, a staple is L^{s}. In some embodiments, L^{s1} is L^{a} of an amino acid residue as described herein. In some embodiments, L^{s1} is L as described herein. For example, in some embodiments, one or more methylene units of L are independently replaced with —C(O)—, —N(R′)—, -Cy- or —O—. In some embodiments, L is —N(R′)—C(O)—(CH₂)ₙ—O—CH₂—, wherein n is 1-10. In some embodiments, L is —C(O)—(CH₂)ₙ—O—CH₂—, wherein n is 1-10. In some embodiments, L is —N(R′)—C(O)—(CH₂)₂O—CH₂—. In some embodiments, L is —C(O)—(CH₂)₂O—CH₂—. In some embodiments, L is —N(R′)—C(O)—(CH₂)₃O—CH₂—. In some embodiments, L is —C(O)—(CH₂)₃O—CH₂—. In some embodiments, L is —N(R′)—C(O)-(1,2-phenylene)-O—CH₂—. In some embodiments, L is —C(O)-(1,2-phenylene)-O—CH₂—. In some embodiments, one or more methylene units of L are replaced with —C(R′)₂—. In some embodiments, one or more methylene units of L are replaced with —CHR′—. In some embodiments, R′ (e.g., of —N(R′)—, —C(R′)₂—, etc.) and another group that can be R, e.g., R^{a1}, R^{a2}, R^{a3} etc. of an amino acid residue (e.g., X¹) are taken together with their intervening atoms to form an optionally substituted 3-10 membered ring having 0-5 heteroatoms as described herein. In some embodiments, R′ (e.g., of —N(R′)—, —C(R′)₂—, etc.) of a staple and another group that can be R, e.g., R^{a1}, R^{a2}, R^{a3}, etc. of an amino acid residue to which the staple is bonded to (e.g., X¹) are taken together with their intervening atoms to form an optionally substituted 3-10 membered ring having 0-5 heteroatoms as described herein. In some embodiments, R′ (e.g., of —N(R′)—, —C(R′)₂—, etc.) of a staple and another group that R^{a1} of an amino acid residue to which the staple is bonded to (e.g., X¹) are taken together with their intervening atoms to form an optionally substituted 3-10 membered ring having 0-5 heteroatoms as described herein. In some embodiments, R′ (e.g., of —N(R′)—, —C(R′)₂—, etc.) of a staple and another group that R^{a2} of an amino acid residue to which the staple is bonded to (e.g., X¹) are taken together with their intervening atoms to form an optionally substituted 3-10 membered ring having 0-5 heteroatoms as described herein. In some embodiments, R′ (e.g., of —N(R′)—, —C(R′)₂—, etc.) of a staple and another group that R^{a3} of an amino acid residue to which the staple is bonded to (e.g., X¹) are taken together with their intervening atoms to form an optionally substituted 3-10 membered ring having 0-5 heteroatoms as described herein. In some embodiments, a formed ring is a ring existed in an amino acid residue, e.g., X¹.

In some embodiments, L^{s3} is L as described herein. In some embodiments, L^{s3} is L^{a} of an amino acid residue as described herein. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is —CH₂—. In some embodiments, L is —CH₂—N(R′)—CH₂—. In some embodiments, R′ is Bn. In some embodiments, R′ is —C(O)R. In some embodiments, R is phenyl. In some embodiments, R is t-butyl. In some embodiments, R is cyclohexyl.

In some embodiments, L^{s2} is optionally substituted —CH═CH—. In some embodiments, L^{s2} is optionally substituted —CH₂—CH₂—. In some embodiments, L^{s2} is —CH₂—CH₂—.

As demonstrated herein, in some embodiments, a staple is bonded to two carbon backbone atoms. In some embodiments, it is independently bonded to an alpha carbon atom of an amino acid residue at each end. In some embodiments, it is bonded to a nitrogen backbone atom (e.g., of an alpha-amino group) and a carbon backbone atom (e.g., an alpha-carbon atom). In some embodiments, it is bonded to two nitrogen backbone atoms (e.g., in some embodiments, each independently of an alpha-amino group).

In some embodiments, X¹ is the 1′ amino acid from the N-terminus. In some embodiments, an amino group of X¹ is a tertiary amine. In some embodiments, an amino group of X¹ is a primary or secondary amine. In some embodiments, an amino group of X¹ is capped. In some embodiments, a capping group is R′ as described herein. In some embodiments, a capping group is —C(O)R wherein R is as described herein. In some embodiments, R is optionally substituted C₁₋₆ aliphatic. In some embodiments, R is optionally substituted C₁₋₆ alkyl. In some embodiments, R is methyl.

In some embodiments, X¹ interacts with Val349 of beta-catenin or an amino acid residue corresponding thereto.

In some embodiments, X¹ is or comprises a residue of an amino acid or a moiety selected from Table A-IV.

Various types of amino acid residues can be used for X², e.g., a residue of an amino acid of formula A-I, A-II, A-III, A-IV, A-V, A-VI, etc. or a salt thereof in accordance with the present disclosure. In some embodiments, X² is —N(R^{a1})-L^{a1}-C(R^{a2})(R^{a3})-L^{a2}-C(O)—, wherein each variable is independently as described herein. In some embodiments, X² is —N(R^{a1})—C(R^{a2})(R^{a3})—C(O)—, wherein each variable is independently as described herein. In some embodiments, X² is —N(R^{a1})—C(R^{a2})H—C(O)—, wherein each variable is independently as described herein. In some embodiments, R^{a1} is —H. In some embodiments, R^{a3} is —H.

In some embodiments, X² is a residue of amino acid (e.g., of formula A-I, A-II, A-III, A-IV, A-V, A-VI, etc. or a salt thereof) that comprises an acidic or polar group. In some embodiments, X² is a residue of amino acid whose side chain comprises an acidic group (in some embodiments, may be referred to as an “acidic amino acid residue”).

In some embodiments, an amino acid residue whose side chain comprises an acidic group comprises —COOH in its side chain. In some embodiments, it is a residue of an amino acid having the structure of formula A-IV or a salt thereof. In some embodiments, it is a residue of amino acid having the structure of formula PA, PA-a, PA-b, PA-c, etc. In some embodiments, R^{PA} is —H and R^{PS} and R^{PC} are —OH. In some embodiments, it is —N(R^{a1})-L^{a1}-C(-L^{a}-COOH)(R^{a3})-L^{a2}-C(O)—. In some embodiments, it is —NH-L^{a1}-C(-L^{a}-COOH)(R^{a3})-L^{a2}-C(O)—. In some embodiments, it is —NH—CH(-L^{a}-COOH)—C(O)—.

As described herein, L^{a} is L as described herein. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n- wherein n is 1-10. In some embodiments, L is —(CH₂)n-. In some embodiments, L is —CH₂—. In some embodiments, L is —(CH₂)₂—. In some embodiments, L is —(CH₂)₃—. In some embodiments, L is —(CH₂)₄—.

In some embodiments, an acidic amino acid residue is Asp. In some embodiments, it is Glu. Other acidic amino acid residues are described herein and can be utilized at various amino acid residue positions.

In some embodiments, X² is a residue of Asp, Glu, Aad, SbMeAsp, RbMeAsp, aMeDAsp, or OAsp. In some embodiments, X² is a residue of Asp, Glu, or Aad. In some embodiments, X² is a residue of Asp. In some embodiments, X² is a residue of Glu. In some embodiments, X² is a residue of Aad. In some embodiments, X² is a residue of SbMeAsp. In some embodiments, X² is a residue of RbMeAsp. In some embodiments, X² is a residue of aMeDAsp. In some embodiments, X² is a residue of OAsp.

In some embodiments, X² is a residue of amino acid (e.g., of formula A-I, A-II, A-III, A-IV, A-V, A-VI, etc. or a salt thereof) whose side chain comprises a polar group (in some embodiments, may be referred to as a “polar amino acid residue”; in some embodiments, it does not include amino acid residue whose side chains are electrically charged at, e.g., about pH 7.4).

In some embodiments, an amino acid residue whose side chain comprises a polar group is —N(R^{a1})-L^{a1}-C(R^{a2})(R^{a3})-L^{a2}-C(O)—. In some embodiments, an amino acid residue whose side chain comprises a polar group is —N(R^{a1})—C(R^{a2})(R^{a3})—C(O)—. In some embodiments, an amino acid residue whose side chain comprises an amide group, e.g., —C(O)N(R′)₂ such as —CONH₂. In some embodiments, R^{a2} is -L^{a}-C(O)N(R′)₂ wherein each variable is independently as described herein. In some embodiments, R^{a2} is -L^{a}-C(O)NH₂ wherein L is independently as described herein. In some embodiments, L^{a} is L′ as described herein. In some embodiments, R^{a3} is H. In some embodiments, such a polar amino acid residue is Asn. In some embodiments, it is MeAsn. In some embodiments, an amino acid residue whose side chain comprises a polar group is an amino acid residue whose side chain comprises —OH. In some embodiments, R^{a2} is -L^{a}-OH wherein each variable is independently as described herein. In some embodiments, R^{a2} is -L^{a}-OH wherein L is independently as described herein. In some embodiments, L^{a} is L′ as described herein. For example, in some embodiments, such an amino acid residue is a residue of Hse, Ser, aThr, or Thr. In some embodiments, it is a residue of Hse, Ser, or aThr. In some embodiments, it is a residue of Hse. In some embodiments, it is a residue of Ser. In some embodiments, it is a residue of aThr. In some embodiments, it is a residue of Thr. Other polar amino acid residues are described herein and can be utilized at various amino acid residue positions.

For example, in some embodiments, X² is a residue of Asn. In some embodiments, X² is a residue of MeAsn. In some embodiments, X² is a residue of Hse, Ser, aThr, or Thr. In some embodiments, X² is a residue of Hse, Ser, or aThr. In some embodiments, X² is a residue of Hse. In some embodiments, X² is a residue of Ser. In some embodiments, X² is a residue of aThr. In some embodiments, X² is a residue of Thr.

In some embodiments, X² is Asp, Ala, Asn, Glu, Npg, Ser, Hse, Val, S5, S6, AcLys, TfeGA, aThr, Aad, Pro, Thr, Phe, Leu, PL3, Gln, isoGlu, MeAsn, isoDAsp, RbGlu, SbGlu, AspSH, Ile, SbMeAsp, RbMeAsp, aMeDAsp, OAsp, 3COOHF, NAsp, 3Thi, NGlu, isoDGlu, BztA, Tle, Aib, MePro, Chg, Cha, or DipA.

In some embodiments, X² is or comprises a residue of an amino acid or a moiety selected from Table A-IV.

In some embodiments, X² interacts with Gly307 of beta-catenin or an amino acid residue corresponding thereto. In some embodiments, X² interacts with Lys312 of beta-catenin or an amino acid residue corresponding thereto. In some embodiments, X² interacts with each of Gly307 and Lys312 of beta-catenin or an amino acid residue corresponding thereto.

Various types of amino acid residues can be used for X³, e.g., a residue of an amino acid of formula A-I, A-II, A-III, A-IV, A-V, A-VI, etc. or a salt thereof in accordance with the present disclosure. In some embodiments, X³ is —N(R^{a1})-L^{a1}-C(R^{a2})(R^{a3})-L^{a2}-C(O)—, wherein each variable is independently as described herein. In some embodiments, X³ is —N(R^{a1})—C(R^{a2})(R^{a3})—C(O)—, wherein each variable is independently as described herein. In some embodiments, X³ is —N(R^{a1})—C(R^{a2})H—C(O)—, wherein each variable is independently as described herein. In some embodiments, R^{a1} is —H. In some embodiments, R^{a3} is —H.

In some embodiments, L^{a} is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is —CH₂—. In some embodiments, L is —CH₂—N(R′)—CH₂—. In some embodiments, R′ is Bn. In some embodiments, R′ is —C(O)R. In some embodiments, R is phenyl. In some embodiments, R is t-butyl. In some embodiments, R is cyclohexyl.

In some embodiments, X³ is a hydrophobic amino acid residue.

In some embodiments, a hydrophobic amino acid residue is an amino acid residue whose side chain is an optionally substituted aliphatic group. In some embodiments, it is a residue of an amino acid whose side chain is optionally substituted C₁₋₁₀ alkyl. In some embodiments, it is a residue of an amino acid whose side chain is C₁₋₁₀ alkyl. In some embodiments, it is a residue of an amino acid whose side chain is C₁-10 aliphatic optionally substituted with one or more non-polar and non-charged groups. In some embodiments, it is a residue of an amino acid whose side chain is C₁₋₁₀ alkyl optionally substituted with one or more non-polar and non-charged groups. In some embodiments, it is a residue of an amino acid whose side chain is C₁₋₁₀ aliphatic optionally substituted with one or more hydrophobic substituents. In some embodiments, it is a residue of an amino acid whose side chain is C₁₋₁₀ aliphatic. In some embodiments, it is a residue of an amino acid whose side chain is C₁₋₁₀ alkyl. Various hydrophobic amino acid residues can be utilized in accordance with the present disclosure.

In some embodiments, a hydrophobic amino acid residue, e.g., X³, has the structure of —NH₂—C(R^{a2})(R^{a3})—C(O)— or —NH—C(R^{a2})H—C(O)— wherein each variable is independently as described herein. As described herein, R^{a2} is -L^{a}-R′. In some embodiments, R′ is R as described herein. In some embodiments, R is optionally substituted group selected from C₁₋₁₀ aliphatic, phenyl, 10-membered aryl, and 5-10 membered heteroaryl having 1-5 heteroatoms. In some embodiments, each substituent, if any, is independently a non-polar group. In some embodiments, R is optionally substituted C₁₋₁₀ aliphatic. In some embodiments, R is optionally substituted C₁₋₁₀ alkyl. In some embodiments, R is C₁₋₁₀ aliphatic. In some embodiments, R is C₁₋₁₀ alkyl. For example, in some embodiments, R is methyl. In some embodiments, R is isopropyl. In some embodiments, R is 1-methylpropyl. In some embodiments, R is 2-methylpropyl. In some embodiments, R is optionally substituted aryl. In some embodiments, R is aryl. In some embodiments, R is optionally substituted phenyl. In some embodiments, R is phenyl. In some embodiments, R is optionally substituted 5-6 membered heteroaryl having 1-4 heteroatoms. In some embodiments, R is optionally substituted 5-6 membered heteroaryl having 1 heteroatom. In some embodiments, R is 5-6 membered heteroaryl having 1-4 heteroatoms. In some embodiments, R is 5-6 membered heteroaryl having 1 heteroatom. In some embodiments, R is optionally substituted 9-10 membered heteroaryl having 1-5 heteroatoms. In some embodiments, R is optionally substituted 9-10 membered heteroaryl having 1 heteroatom. In some embodiments, R is 9-10 membered heteroaryl having 1-4 heteroatoms. In some embodiments, R is 9-10 membered heteroaryl having 1 heteroatom. In some embodiments, a heteroatom is nitrogen. In some embodiments, a heteroatom is oxygen. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, a hydrophobic amino acid residue is a residue of Ala, Val, Ile, Leu, Met, Phe, Tyr, Trp, etc. Other hydrophobic amino acid residues are described herein and can be utilized at various amino acid residue positions.

In some embodiments, X³ comprises a side chain comprising a cycloaliphatic group (e.g., a 4-, 5-or 6-membered cycloalkyl group). In some embodiments, X³ is a residue of Npg, Leu, Cha, Val, nLeu, Ile, CypA, CyLeu, Chg, DiethA, Ala, Aib, OctG, or Cba. In some embodiments, X³ is a residue of Npg, Leu, or Cha. In some embodiments, X³ is a residue of Npg. In some embodiments, X³ is a residue of Leu. In some embodiments, X³ is a residue of Cha. In some embodiments, X³ is a residue of Val. In some embodiments, X³ is a residue of nLeu. In some embodiments, X³ is a residue of Ile. In some embodiments, X³ is a residue of CypA. In some embodiments, X³ is a residue of CyLeu. In some embodiments, X³ is a residue of Chg. In some embodiments, X³ is a residue of DiethA. In some embodiments, X³ is a residue of Ala. In some embodiments, X³ is a residue of Aib. In some embodiments, X³ is a residue of OctG. In some embodiments, X³ is a residue of Cba.

In some embodiments, X³ comprises a side chain which is or comprises an optionally substituted aromatic group (in some embodiments, may be referred to as an “aromatic amino acid residue”).

In some embodiments, an aromatic amino acid residue has a side chain which is or comprises an optionally substituted aromatic group. In some embodiments, an aromatic amino acid residue, e.g., X³, has the structure of —NH₂—C(R^{a2})(R^{a3})—C(O)— or —NH—C(R^{a2})H—C(O)— wherein each variable is independently as described herein, and R^{a2} comprises an optionally substituted aromatic group.

In some embodiments, an aromatic amino acid residue has a side chain which is or comprises an optionally substituted aromatic group, wherein each substituent of the aromatic group is independently halogen. In some embodiments, it comprises a side chain which is or comprises two optionally substituted aromatic groups. In some embodiments, it comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, it is a residue of an amino acid of formula A-I or a salt thereof. In some embodiments, an amino acid residue has the structure of —NH—C(R^{a2})(R^{a3})—C(O)— or —NH—CH(R^{a3})—C)O)—. As described herein, R^{a3} is -L^{a}-R′ wherein each variable is independently as described herein. In some embodiments, R′ is an optionally substituted group selected from phenyl, 10-membered bicyclic aryl, 5-6 membered heteroaryl having 1-4 heteroatoms, and 9-10 membered bicyclic heteroaryl having 1-5 heteroatoms. In some embodiments, each substituent is independently halogen or —OH. In some embodiments, R′ is optionally substituted phenyl. In some embodiments, R′ is phenyl. In some embodiments, R′ is optionally substituted aryl. In some embodiments, R′ is aryl. In some embodiments, R′ is optionally substituted 5-membered heteroaryl having 1-4 heteroatoms. In some embodiments, R′ is optionally substituted 5-membered heteroaryl having 1 heteroatom. In some embodiments, R′ is 5-6 membered heteroaryl having 1-4 heteroatoms. In some embodiments, R′ is 5-6 membered heteroaryl having 1 heteroatom. In some embodiments, R′ is optionally substituted 9-10 membered heteroaryl having 1-5 heteroatoms. In some embodiments, R′ is optionally substituted 9-10 membered heteroaryl having 1 heteroatom. In some embodiments, R′ is 9-10 membered heteroaryl having 1-4 heteroatoms. In some embodiments, R′ is 9-10 membered heteroaryl having 1 heteroatom. In some embodiments, a heteroatom is nitrogen. In some embodiments, a heteroatom is oxygen. In some embodiments, a heteroatom is sulfur. In some embodiments, L^{a} is a covalent bond. In some embodiments, L^{a} is optionally substituted —(CH₂)n- wherein n is 1-10. In some embodiments, L^{a} is —(CH₂)n-. In some embodiments, n is 1. In some embodiments, n is 2. In some embodiments, n is 3. In some embodiments, L^{a} is —CH(Ph)-. In some embodiments, an aromatic amino acid residue is Phe. In some embodiments, an aromatic amino acid residue is Tyr. In some embodiments, an aromatic amino acid residue is Trp. Other aromatic amino acid residues are described herein and can be utilized at various amino acid residue positions.

In some embodiments, X³ is a residue of Phe. In some embodiments, X³ is a residue of Pff. In some embodiments, X³ is a residue of Tyr. In some embodiments, X³ is a residue of Trp. In some embodiments, X³ is a residue of Phg. In some embodiments, X³ is a residue of DipA.

In some embodiments, X³ is or comprises a residue of an amino acid or a moiety selected from Table A-I, Table A-II, Table A-III and Table A-IV.

In some embodiments, X³ is a residue of an amino acid suitable for stapling. In some embodiments, X³ is a residue of an amino acid comprising a double bond, e.g., a terminal olefin, suitable for stapling. In some embodiments, X³ is a residue of an amino acid having the structure of A-II, A-III, etc. or a salt thereof. In some embodiments, X³ is —N(R^{a1})-L^{a1}-C(-L^{a}-CH═CH₂)(R^{a3})-L^{a2}-C(O)—, wherein each variable is independently as described herein. In some embodiments, X³ is —N(R^{a1})—C(-L^{a}-CH═CH₂)(R^{a3})—C(O)—, wherein each variable is independently as described herein. In some embodiments, X³ is residue of AllylGly

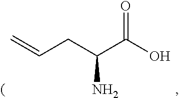

residue being

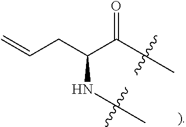 [SMILES: *N[C@@H](CC=C)C(*)=O]

In some embodiments, X³ is [Bn][Allyl]Dap

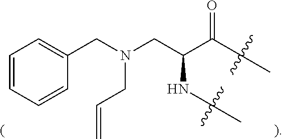 [SMILES: *N[C@@H](CN(CC=C)Cc1ccccc1)C(*)=O]

In some embodiments, X³ is [Phc][Allyl]Dap

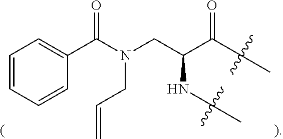 [SMILES: *N[C@@H](CN(CC=C)C(=O)c1ccccc1)C(*)=O]

In some embodiments, X³ is [Piv][Allyl]Dap

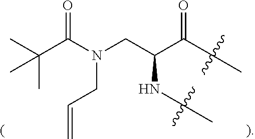 [SMILES: *N[C@@H](CN(CC=C)C(=O)C(C)(C)C)C(*)=O]

In some embodiments, X³ is [CyCO][Allyl]Dap

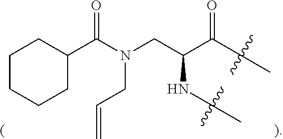 [SMILES: *N[C@@H](CN(CC=C)C(=O)C1CCCCC1)C(*)=O]

In some embodiments, X³ is stapled. In some embodiments, X³ is stapled with X¹ (e.g., through olefin metathesis wherein both X¹ and X³ comprises —CH═CH₂). In some embodiments, a staple has the structure of -L^{s1}-L^{s2}-L^{s3}-, wherein each variable is as described herein. In some embodiments, L^{s}I is L^{a} of one stapled amino acid residue (e.g., X¹) and L^{s3} is L^{a} of the other stapled amino acid residue (e.g., X³). For example, in some embodiments, L^{s} is —C(O)—(CH₂)n-L^{s2}-(CH₂)n-, wherein each variable is independently as described herein. In some embodiments, L^{s} is —C(O)—(CH₂)n-L^{s2}-CH₂—N(R′)— CH₂—, wherein each variable is independently as described herein. In some embodiments, n is 1. In some embodiments, n is 2. In some embodiments, n is 3. In some embodiments, L^{s} is —C(O)-Cy-O—CH₂-L^{s2}-CH₂—, each variable is independently as described herein. In some embodiments, L^{s} is —C(O)-Cy-O—CH₂-L^{s2}-CH₂—N(R′)—CH₂—, each variable is independently as described herein. In some embodiments, -Cy- is optionally substituted phenylene. In some embodiments, -Cy- is 1,2-phenylene. In some embodiments, R′ is Bn. In some embodiments, R′ is —C(O)R. In some embodiments, R is phenyl. In some embodiments, R is t-butyl. In some embodiments, R is cyclohexyl. In some embodiments, L^{s2} is optionally substituted —CH═CH—. In some embodiments, L^{s2} is —CH═CH—. In some embodiments, L^{s2} is optionally substituted —CH₂—CH₂—. In some embodiments, L^{s2} is —CH₂—CH₂—. In some embodiments, one end of a staple, e.g., L^{s}, is bonded to a backbone nitrogen atom (e.g., of an alpha amino group, at —C(O)— of a staple) and the other end is bonded to a backbone carbon atom (e.g., an alpha carbon atom, at —CH₂— of a staple).

In some embodiments, an amino acid residue suitable for stapling, e.g., X³, is of an amino acid of formula V or VI or a salt thereof. In some embodiments, such an amino acid residue is —N(R^{a1})-L^{a1}-C(-L^{a}-R^{SP1})(R^{a3})-L^{a2}-C(O)—, wherein each variable is independently as described herein. In some embodiments, such an amino acid residue is —N(R^{a1})—C(-L^{a}-R^{SP1})(R^{a3})—C(O)—, wherein each variable is independently as described herein. In some embodiments, a reactive group R^{SP1} is —COOH. In some embodiments, an amino acid suitable for stapling is an amino acid of formula IV or a salt thereof. In some embodiments, such an amino acid is GlnR. In some embodiments, such an amino acid residue can be stapled with another amino acid residue suitable for stapling, e.g., that comprises a R^{SP1} group that is —NH₂ (e.g., in Lys).

In some embodiments, X³ is GlnR.

In some embodiments, X³ is stapled with X⁷. In some embodiments, a side chain of X³ comprises —COOH that forms a staple with, e.g., a side chain of another amino acid comprising an amino group (e.g., Lys).

As described herein, in some embodiments, a staple, e.g., L^{s}, comprises —C(O)N(R′)— wherein R′ is as described herein. In some embodiments, R′ is —H. In some embodiments, a staple, e.g., L^{s} has the structure of -L″-C(O)N(R′)-L^{s3}-, wherein each variable is independently as described herein. In some embodiments, L^{s}I is L as described herein. In some embodiments, L^{s3} is L as described herein. In some embodiments, L^{s}I is L^{a} as described herein of one amino acid residue of a stapled pair. In some embodiments, L^{s}I is L^{a} as described herein of the other amino acid residue of a stapled pair. In some embodiments, L^{s}I is independently an optionally substituted bivalent linear or branched C₁₋₁₀ hydrocarbon chain. In some embodiments, L^{s3} is independently an optionally substituted bivalent linear or branched C₁₋₁₀ hydrocarbon chain. In some embodiments, each of L^{s1} and L^{s3} is independently an optionally substituted bivalent linear or branched C₁₋₁₀ hydrocarbon chain. In some embodiments, each of L^{s1} and L^{s3} is independently —(CH₂)n-, wherein n is 1-10. In some embodiments, L^{s1} is —CH₂—. In some embodiments, L^{s3} is —(CH₂)₃—.

In some embodiments, L^{s2} is L as described herein. In some embodiments, L is or comprises —C(O)N(R′)— wherein R′ is as described herein. In some embodiments, L is or comprises —C(O)NH—.

In some embodiments, L^{s} is —(CH₂)ₙ₁—C(O)NH—(CH₂)ₙ₂—, wherein each of n1 and n2 is independently n as described herein. In some embodiments, L^{s} is —(CH₂)₂—C(O)NH—(CH₂)₄—. In some embodiments, such a staple connects X³ and X⁷. In some embodiments, such a staple may connect other pairs of stapled amino acid residues.

In some embodiments, X³ is a residue of amino acid that comprises an acidic or polar group. In some embodiments, X³ is a residue of amino acid whose side chain comprises an acidic group, e.g., a —COOH group or a salt form thereof (e.g., a compound of formula A-IV, PA, PA-a, PA-b, PA-c, etc.). In some embodiments, X³ is —N(R^{a1})-L^{a1}-C(-L^{a}-COOH)(R^{a3})-L^{a2}-C(O)— wherein each variable is independently as described herein. In some embodiments, X³ is —N(R^{a1})—C(-L^{a}-COOH)(R^{a3})—C(O)— wherein each variable is independently as described herein. In some embodiments, X³ is a residue of Asp. In some embodiments, X³ is a residue of amino acid whose side chain comprises —OH. For example, in some embodiments, X³ is a residue of Tyr. In some embodiments, X³ is a residue of Ser.

In some embodiments, X³ is a residue selected from Npg, Leu, Cha, AllylGly, GlnR, Val, nLeu, Asp, [Bn][Allyl]Dap, [Phc][Allyl]Dap, Ile, Phe, CypA, CyLeu, Chg, Pff, DiethA, Ala, Tyr, Trp, Ser, Aib, Phg, OctG, Cba, MorphNva, F2PipNva, [Piv][Allyl]Dap, and [CyCO][Allyl]Dap.

In some embodiments, X³ is a residue of Npg, Ile, Asp, Cha, DipA, Chg, Leu, B5, Cba, S5, Ala, Glu, AllylGly, nLeu, Ser, B6, Asn, B4, GlnR, Val, [Phc][Allyl]Dap, Hse, [Bn][Allyl]Dap, 1MeK, R5, Phe, CypA, CyLeu, Pff, DiethA, Tyr, Trp, Aib, Phg, OctG, MorphNva, F2PipNva, [Piv][Allyl]Dap, [CyCO][Allyl]Dap, Lys, or S3. In some embodiments, X³ is Npg. In some embodiments, X³ is Leu. In some embodiments, Npg provides better properties and/or activities than, e.g., Ala.

In some embodiments, X³ interacts with Tyr306 of beta-catenin or an amino acid residue corresponding thereto.

In some embodiments, X³ is or comprises a residue of an amino acid or a moiety selected from Table A-IV.

Various types of amino acid residues can be used for X⁴, e.g., a residue of an amino acid of formula A-I, A-II, A-III, A-IV, A-V, A-VI, etc. or a salt thereof in accordance with the present disclosure. In some embodiments, X⁴ is a residue of an amino acid of formula A-I, A-II, A-III, A-IV, A-V, A-VI, etc. or a salt thereof. In some embodiments, X⁴ is a residue of an amino acid of formula A-II or salt thereof. In some embodiments, X⁴ is a residue of an amino acid of formula A-III or salt thereof. In some embodiments, X⁴ is a residue of an amino acid of formula A-IV or salt thereof. In some embodiments, X⁴ is a residue of an amino acid of formula A-V or salt thereof. In some embodiments, X⁴ is a residue of an amino acid of formula A-VI or salt thereof. In some embodiments, X⁴ is —N(R^{a1})-L^{a1}-C(R^{a2})(R^{a3})-L^{a2}-C(O)—, wherein each variable is independently as described herein. In some embodiments, X⁴ is —N(R^{a1})—C(R^{a2})(R^{a3})—C(O)— wherein each variable is independently as described herein. In some embodiments, X⁴ is —N(R^{a1})—C(R^{a2})H—C(O)— wherein each variable is independently as described herein. In some embodiments, R^{a2} is -L^{a}-CH═CH₂, wherein L^{a} is as described herein. In some embodiments, R^{a3} is -L^{a}-CH═CH₂, wherein L^{a} is as described herein. In some embodiments, X⁴ is —N(R^{a1})-L^{a1}-C(-L^{a}-R^{SP1})(-L^{a}-R^{SP2})-L^{a2}-C(O)— wherein each variable is independently as described herein. In some embodiments, X⁴ is —N(R^{a1})—C(-L^{a}-R^{SP1})(-L^{a}-R^{SP2})—C(O)— wherein each variable is independently as described herein. In some embodiments, R^{a1} is —H. In some embodiments, R^{a3} is —H.

In some embodiments, each of R^{SP1} and R^{SP2} is or comprises independently optionally substituted —CH═CH₂. In some embodiments, each of R^{SP1} and R^{SP2} is independently —CH═CH₂. In some embodiments, each of -L^{a}-connected R^{SP1} or R^{SP2} is independent L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—.

In some embodiments, X⁴ is or comprises a residue of an amino acid or a moiety selected from Table A-I, Table A-II, Table A-III and Table A-IV.

In some embodiments, X⁴ is residue of an amino acid suitable for stapling. In some embodiments, X⁴ is a residue of an amino acid which comprises two functional groups suitable for stapling. In some embodiments, X⁴ is a residue of an amino acid which comprises one and only one functional group suitable for stapling. In some embodiments, X⁴ is a residue of an amino acid which comprises two olefins, e.g., two terminal olefins. In some embodiments, X⁴ is a residue of an amino acid which comprises one and only one double bond for stapling, e.g., a terminal olefin. In some embodiments, X⁴ is a residue of an amino acid which has the structure of formula A-I, A-II, A-III, etc., wherein both R^{a2} and R^{a3} are independently -L^{a}-CH═CH₂, wherein each L^{a} is independently as described herein. In some embodiments, X⁴ is a residue of an amino acid which has the structure of formula A-I, A-II, A-III, etc., wherein only one of R^{a2} and R^{a3} is -L^{a}-CH═CH₂, wherein each L^{a} is independently as described herein. In some embodiments, each L^{a} is independently optionally substituted bivalent C₁₋₁₀ alkylene or heteroalkylene. In some embodiments, each L^{a} is independently optionally substituted —(CH₂)n- wherein n is 1-10. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, X⁴ is a residue of B5, R⁵, R⁴, or R⁶. In some embodiments, X⁴ is a residue of B5 or R⁵. In some embodiments, X⁴ is a residue of B5. In some embodiments, X⁴ is residue of R⁵. In some embodiments, X⁴ is a residue of R⁴. In some embodiments, X⁴ is a residue of R⁶.

In some embodiments, X⁴ is stapled. In some embodiments, X⁴ is connected to two residues independently through two staples (e.g., when X⁴ is B5). In some embodiments, X⁴ is staple with X¹, and X⁴ is stapled with X¹¹.

As described herein, various staples may be utilized for connecting stapled amino acid residues. In some embodiments, a staple is L^{s} as described herein. In some embodiments, each staple connected to X⁴ is independently L^{s} as described herein.

In some embodiments, L^{s} is -L^{s1}-L^{s2}-L^{s3}-, wherein each variable is independently as described herein. In some embodiments, one of L^{s1} and L^{s3} is L^{a} of one of two stapled amino acid residues, and the other is L^{a} of the other of two stapled amino acid residues. In some embodiments, L^{s3} is L^{a} of X⁴, e.g., when X⁴ is stapled with an amino acid residue to its N-terminus side (e.g., X¹). In some embodiments, L^{s1} is L^{a} of X⁴, e.g., when X⁴ is stapled with an amino acid residue to its C-terminus side (e.g., X¹). In some embodiments, L^{s1} is L^{a} of X¹, and L^{s3} is L^{a} of X⁴. In some embodiments, L^{s1} is L^{a} of X⁴, and L^{s3} is L^{a} of X¹ In some embodiments, two staples are bonded to X⁴, wherein a first staple staples X⁴ with an amino acid residue to the N-terminus side of X⁴ (an amino acid residue to a N-terminus side of a reference amino acid residue may be referred to as “N-direction amino acid residue” of the reference amino acid residue, e.g., X¹ is a N-direction amino acid residue of X⁴), wherein the first staple is L^{s} having the structure of -L^{s1}-L^{s2}-L^{s3}-, wherein L^{s1} is L^{a} of the N-direction amino acid residue, and L^{s3} is L^{a} of X⁴, and wherein a second staple staples X⁴ with an amino acid residue to the C-terminus side of X⁴ (an amino acid residue to a C-terminus side of a reference amino acid residue may be referred to as “C-direction amino acid residue” of the reference amino acid residue, e.g., X¹ is a C-direction amino acid residue of X⁴), wherein the second staple is L^{s}having the structure of -L^{s1}-L^{s2}-L^{s3}-, wherein L^{s3} is L^{a} of the C-direction amino acid residue, and L^{s1} is L^{a} of X⁴. Various embodiments of L^{a} are described herein and can be utilized for various amino acid residues including X⁴ and N-direction (e.g., X¹) and C-direction (e.g., X¹) amino acid residues. For example, in some embodiments, for X⁴ each L^{a} is —(CH₂)₃—.

As described herein, in some embodiments, L^{s2} is optionally substituted —CH═CH—. In some embodiments, L^{s2} is —CH═CH—. In some embodiments, L^{s2} is optionally substituted —CH₂—CH₂—. In some embodiments, L^{s2} is —CH₂—CH₂—.

In some embodiments, as described herein, each staple is independently bonded to two alpha carbon atoms of two stapled amino acid residues.

In some embodiments, X⁴ is stapled with two amino acid residues, e.g., X¹ and X¹¹. In some embodiments, X⁴ is stapled with only one residue, e.g., X¹¹ (e.g., when X⁴ is a residue of R⁵, R⁴, or R⁶). In some embodiments, X⁴ is —N(R^{a1})-L^{a1}-C(-L^{a}-CH═CH₂)(R^{a3})-L^{a2}-C(O)— wherein each variable is independently as described herein. In some embodiments, X⁴ is —N(R^{a1})—C(-L^{a}-CH═CH₂)(R^{a3})—C(O)— wherein each variable is independently as described herein. In some embodiments, X⁴ is a residue of R⁴. In some embodiments, X⁴ is a residue of R⁵. In some embodiments, X⁴ is a residue of R⁶.

In some embodiments, a staple is L^{s} as described herein. For example, in some embodiments, L^{s1} is L^{a} of a first amino acid residue of two stapled amino acid residues, e.g., X⁴, and L^{s3} is L^{a} of a second amino acid residue of two stapled amino acid residues, e.g., X¹¹, wherein a second amino acid residue (e.g., X¹) is a C-direction amino acid residue of a first amino acid residue (e.g., X⁴).

In some embodiments, X⁴ is not stapled (e.g., when other residues are optionally stapled, in pre-stapling agents, etc.).

In some embodiments, X⁴ is B5, Npg, Asp, R⁵, Ile, Ala, Cha, Chg, Ser, Leu, R⁴, R⁶, Phe, or S5.

Various types of amino acid residues can be used for X⁵, e.g., a residue of an amino acid of formula A-I, A-II, A-III, A-IV, A-V, A-VI, etc. or a salt thereof in accordance with the present disclosure. In some embodiments, X⁵ is —N(R^{a1})-L^{a1}-C(R^{a2})(R^{a3})-L^{a2}-C(O)—, wherein each variable is independently as described herein. In some embodiments, X⁵ is —N(R^{a1})—C(R^{a2})(R^{a3})—C(O)—, wherein each variable is independently as described herein. In some embodiments, X⁵ is —N(R^{a1})—C(R^{a2})H—C(O)—, wherein each variable is independently as described herein. In some embodiments, R^{a1} is —H. In some embodiments, R^{a3} is —H.

In some embodiments, X⁵ is a residue of amino acid that comprises an acidic or polar group. In some embodiments, X⁵ is a residue of amino acid whose side chain comprises an acidic group, e.g., a —COOH group or a salt form thereof. In some embodiments, X⁵ is a residue of an amino acid of formula A-IV or a salt thereof. In some embodiments, X⁵ is a residue of an amino acid of formula PA, PA-a, PA-b, PA-c, or a salt thereof. In some embodiments, R^{PA} is —H and R^{PS} and R^{PC} are —OH. In some embodiments, X⁵ is —N(R^{a1})-L^{a1}-C(-L^{a}-COOH)(R^{a3})-L^{a2}-C(O)— wherein each variable is independently as described herein. In some embodiments, X⁵ is —N(R^{a1})—C(-L^{a}-COOH)(R^{a3})—C(O)— wherein each variable is independently as described herein. In some embodiments, L^{a} is L as described herein. For example, in some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is —CH(CH₃)—. In some embodiments, L is an optionally substituted bivalent linear or branched C₁₋₁₀ hydrocarbon chain wherein one or more methylene units of L are independently replaced with —C(O)—, —N(R′)—, -Cy- or —O—.

In some embodiments, X⁵ is a residue of Asp, Glu, Aad, SbMeAsp, or RbMeAsp. In some embodiments, X⁵ is a residue of Asp or Glu. In some embodiments, X⁵ is a residue of Asp. In some embodiments, X⁵ is a residue of Glu. In some embodiments, X⁵ is a residue of Aad. In some embodiments, X⁵ is a residue of SbMeAsp. In some embodiments, X⁵ is a residue of RbMeAsp.

In some embodiments, X⁵ is a residue of amino acid whose side chain comprises a polar group. In some embodiments, X⁵ is a residue of amino acid whose side chain comprises an amide group, e.g., —C(O)N(R′)₂ such as —CONH₂. In some embodiments, R^{a2} is -L^{a}-C(O)N(R′)₂ wherein each variable is independently as described herein. In some embodiments, R^{a2} is -L^{a}-C(O)NH₂ wherein L is independently as described herein. In some embodiments, L^{a} is L′ as described herein. For example, in some embodiments, X⁵ is a residue of Asn. In some embodiments, X⁵ is a residue of MeAsn. In some embodiments, X⁵ is a residue of amino acid whose side chain comprises —OH. For example, in some embodiments, X⁵ is a residue of Hse, aThr, Ser, or Thr. In some embodiments, X⁵ is a residue of Hse or aThr. In some embodiments, X⁵ is a residue of Hse. In some embodiments, X⁵ is a residue of aThr. In some embodiments, X⁵ is a residue of Ser. In some embodiments, X⁵ is a residue of Thr.

In some embodiments, X⁵ is Asp, B5, 3COOHF, Glu, Asn, Npg, Hse, aThr, Aad, Ser, Thr, MeAsn, AspSH, SbMeAsp, RbMeAsp. In some embodiments, X⁵ is Asp. In some embodiments, X⁵ is 3COOHF. In some embodiments, X⁵ is Glu. In some embodiments, X⁵ is B5. In some embodiments, X⁵ is DipA. In some embodiments, X⁵ is Chg.

In some embodiments, X⁵ is or comprises a residue of an amino acid or a moiety selected from Table A-IV.

In some embodiments, X⁵ interacts with Trp383 of beta-catenin or an amino acid residue corresponding thereto. In some embodiments, X⁵ interacts with Arg386 of beta-catenin or an amino acid residue corresponding thereto. In some embodiments, X⁵ interacts with Asn387 of beta-catenin or an amino acid residue corresponding thereto. In some embodiments, X⁵ interacts with Asn387 and Trp383 of beta-catenin or amino acid residues corresponding thereto.

Various types of amino acid residues can be used for X⁶, e.g., a residue of an amino acid of formula A-I, A-II, A-III, A-IV, A-V, A-VI, etc. or a salt thereof in accordance with the present disclosure. In some embodiments, X⁶ is a residue of an amino acid of formula A-I, A-II, A-III, A-IV, A-V, A-VI, etc. or a salt thereof. In some embodiments, X⁶ is —N(R^{a1})-L^{a1}-C(R^{a2})(R^{a3})-L^{a2}-C(O)—, wherein each variable is independently as described herein. In some embodiments, X⁶ is —N(R^{a1})—C(R^{a2})(R^{a3})—C(O)—, wherein each variable is independently as described herein. In some embodiments, X⁶ is —N(R^{a1})—C(R^{a2})H—C(O)—, wherein each variable is independently as described herein. In some embodiments, X⁶ is a residue of an amino acid of formula A-IV or a salt thereof. In some embodiments, X⁶ is a residue of an amino acid of formula PA, PA-a, PA-b, PA-c, or a salt thereof. In some embodiments, R^{PA} is —H and R^{PS} and R^{PC} are —OH. In some embodiments, R^{a1} is —H. In some embodiments, R^{a3} is —H.

In some embodiments, X⁶ is a residue of amino acid that comprises an acidic or polar group. In some embodiments, X⁶ is a residue of amino acid whose side chain comprises an acidic group, e.g., a —COOH group or a salt form thereof. In some embodiments, X⁶ is a residue of an amino acid having the structure of formula A-IV or a salt thereof. In some embodiments, X⁶ is a residue of amino acid having the structure of formula PA, PA-a, PA-b, PA-c, etc. In some embodiments, R^{PA} is —H and R^{PS} and R^{PC} are —OH. In some embodiments, X⁶ is —N(R^{a1})-L^{a1}-C (-L^{a}-COOH)(R^{a3})-L^{a2}-C(O)—. In some embodiments, X⁶ is —NH-L^{a1}-C(-L^{a}-COOH)(R^{a3})-L^{a2}-C(O)—. In some embodiments, X⁶ is —NH—CH(-L^{a}-COOH)—C(O)—.

As described herein, L^{a} is L as described herein. In some embodiments, L is an optionally substituted bivalent linear or branched C₁₋₁₀ hydrocarbon chain wherein one or more methylene units of L are independently replaced with —C(O)—, —N(R′)—, -Cy- or —O—. In some embodiments, L is an optionally substituted bivalent linear C₁₋₁₀ hydrocarbon chain wherein one or more methylene units of L are independently replaced with —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n- wherein n is 1-10. In some embodiments, L is —(CH₂)n-. In some embodiments, L is —CH₂—. In some embodiments, L is —(CH₂)₂—. In some embodiments, L is —(CH₂)₃—. In some embodiments, L is —(CH₂)₄—. In some embodiments, a methylene unit is replaced with -Cy-. In some embodiments, L is —CH₂-Cy-CH₂—. In some embodiments, L is —CH₂-Cy-. In some embodiments, L is —(CH₂)₄-Cy-CH₂—C(CH₃)₂—. In some embodiments, -Cy- is optionally substituted phenylene. In some embodiments, -Cy- is phenylene. In some embodiments, -Cy- is substituted phenylene. In some embodiments, -Cy- is mono-substituted phenylene. In some embodiments, a substituent is —F. In some embodiments, a substituent is optionally substituted C₁₋₆ alkyl. In some embodiments, a substituent is —CF₃. In some embodiments, a substituent is —OH. In some embodiments, phenylene is 1,2-phenylene. In some embodiments, phenylene is 1,3-phenylene. In some embodiments, phenylene is 1,4-phenylene. In some embodiments, a substituent is ortho to the carbon atom closed to —COOH. In some embodiments, it is meta. In some embodiments, it is para. In some embodiments, -Cy- is 1,3-phenylene (e.g., in 3COOHF). In some embodiments, -Cy- is an optionally substituted bivalent 5-10 membered heteroaryl group having 1-5 heteroatoms. In some embodiments, -Cy- is an optionally substituted bivalent 5-membered heteroaryl group having 1-4 heteroatoms. In some embodiments, -Cy- is an optionally substituted bivalent 6-membered heteroaryl group having 1-4 heteroatoms. In some embodiments, -Cy- is optionally substituted

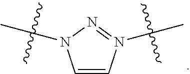

In some embodiments, -Cy- is

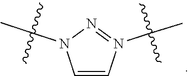

In some embodiments, -Cy- is optionally substituted

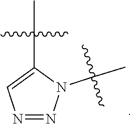 [SMILES: *c1cnnn1*]

In some embodiments, -Cy- is

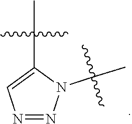 [SMILES: *c1cnnn1*]

In some embodiments, L is bonded to a backbone atom, e.g., an alpha carbon atom, at —CH₂—. In some embodiments, a methylene unit is replaced with —N(R′)— wherein R′ is as described herein. In some embodiments, L is —CH₂—N(R′)—CH₂— wherein R′ is as described herein. In some embodiments, R′ is R as described herein. In some embodiments, R is optionally substituted C₁₋₆ alkyl. In some embodiments, R is —CH₂CF₃.

In some embodiments, X⁶ is a residue of an amino acid of formula PA, PA-a, PA-b, PA-c, or a salt thereof, wherein R^{PA} is —H and R^{PS} and R^{PC} are —OH. In some embodiments, X⁶ is a residue of 3COOHF, TfeGA, Asp, [CH2CMe2CO2H]TriAzDap, Glu, 2OH3COOHF, 40H3COOHF, 4COOHF, 2COOHF, 5F3Me2COOHF, 4F3Me2COOHF, 5F3Me3COOHF, 4F3Me3COOHF, 3F2COOHF, or dGlu. In some embodiments, X⁶ is a residue of 3COOHF, TfeGA, Asp, or [CH2CMe2CO2H]TriAzDap. In some embodiments, X⁶ is a residue of 3COOHF. In some embodiments, X⁶ is a residue of TfeGA. In some embodiments, X⁶ is a residue of Asp. In some embodiments, X⁶ is a residue of [CH2CMe2CO2H]TriAzDap. In some embodiments, X⁶ is a residue of Glu. In some embodiments, X⁶ is a residue of 20H3COOHF. In some embodiments, X⁶ is a residue of 40H3COOHF. In some embodiments, X⁶ is a residue of 4COOHF. In some embodiments, X⁶ is a residue of 2COOHF. In some embodiments, X⁶ is a residue of 5F3Me2COOHF. In some embodiments, X⁶ is a residue of 4F3Me2COOHF. In some embodiments, X⁶ is a residue of 5F3Me3COOHF. In some embodiments, X⁶ is a residue of 4F3Me3COOHF. In some embodiments, X⁶ is a residue of 3F2COOHF. In some embodiments, X⁶ is a residue of dGlu.

In some embodiments, X⁶ is a residue of amino acid whose side chain comprises a polar group. Certain such amino acid residues useful for X⁶ include those described for, e.g., X², X⁵, etc., whose side chain comprise a polar group. In some embodiments, X⁶ is a residue of amino acid whose side chain comprises —OH. For example, in some embodiments, X⁶ is a residue of Thr, Tyr, Ser, aThr, or hTyr. In some embodiments, X⁶ is a residue of Thr. In some embodiments, X⁶ is a residue of Tyr. In some embodiments, X⁶ is a residue of Ser. In some embodiments, X⁶ is a residue of aThr. In some embodiments, X⁶ is a residue of hTyr. In some embodiments, X⁶ is a residue of amino acid whose side chain comprises an amide group, e.g., —C(O)N(R′)₂ such as —CONH₂. In some embodiments, X⁶ is a residue of Asn. In some embodiments, X⁶ is Me2Gln.

In some embodiments, X⁶ is a residue of an amino acid whose side chain is hydrophobic. Certain such amino acid residues include those hydrophobic amino acid residues described for, e.g., X³. In some embodiments, X⁶ is a residue of an amino acid whose side chain is an optionally substituted aliphatic group. In some embodiments, X⁶ is a residue of Val. In some embodiments, X⁶ is a residue of Ala. In some embodiments, X⁶ is a residue of Leu. In some embodiments, X⁶ is a residue of Ile.

As those skilled in the art reading the present disclosure will readily appreciate, amino acid residues of certain properties, structures, etc. described for one position may also be utilized at other positions where amino acid residues of the same properties, structures, etc. can be utilized. For example, when hydrophobic amino acid residues can be utilized at both positions X³ and X⁶, hydrophobic amino acid residues described for X³ can be utilized for X⁶ and vice versa. Similarly, when acidic amino acid residues can be utilized at positions X², X⁵ and X⁶, acidic amino acid residues described for one of them may be utilized at the other two positions as well.

In some embodiments, X⁶ comprises a side chain comprising an optionally substituted aromatic group. Certain such amino acid residues include those amino acid residues whose side chains comprise aromatic groups described for, e.g., X³. In some embodiments, an aromatic group is optionally substituted 5-membered heteroaryl having 1-3 nitrogen atoms. In some embodiments, an aromatic group is optionally substituted 8-10 membered bicyclic aryl or heteroaryl having 1-5 heteroatoms. In some embodiments, an aromatic group is phenyl. In some embodiments, an aromatic group is optionally substituted phenyl. In some embodiments, X⁶ is a residue of His. In some embodiments, X⁶ is a residue of Trp. In some embodiments, X⁶ is a reside of Phe. In some embodiments, X⁶ is a residue of 3cbmf.

In some embodiments, X⁶ is a residue selected from 3COOHF, TfeGA, Asp, [CH2CMe2CO2H]TriAzDap, Glu, 2OH3COOHF, 40H3COOHF, 4COOHF, 2COOHF, 5F3Me2COOHF, 4F3Me2COOHF, 5F3Me3COOHF, 4F3Me3COOHF, 3F2COOHF, dGlu, Thr, Tyr, Ser, aThr, hTyr, Glyn, Lys, Arg, Val, Ala, Leu, Phe, Ile, His, Trp, or 3cbmf. In some embodiments, X⁶ is a residue of Gln. In some embodiments, X⁶ is a residue of Lys. In some embodiments, X⁶ is a residue of Arg.

In some embodiments, X⁶ is 3COOHF, Asp, TfeGA, Aib, Glu, Npg, Gln, [CH2CMe2CO2H]TriAzDap, B5, Thr, Ser, Asn, Ala, Hse, 4BOH2F, 2OH3COOHF, 40H3COOHF, 4COOHF, 2COOHF, His, Tyr, 5F3Me2COOHF, 4F3Me2COOHF, 5F3Me3COOHF, 4F3Me3COOHF, 3F2COOHF, Val, Trp, Arg, dGlu, aThr, hTyr, 3cbmf, Leu, Phe, Lys, Ile, SbMeAsp, bMe2Asp, 3BOH2F, [Ac]Dap, [CH₂CO2H]Acp, [Pfbn]GA, [Tfb]GA, [Succinate]Dap, [Malonate]Dap, [Me2Mal]Dap, [SaiPrSuc]Dap, [SaMeSuc]Dap, or [RaiPrSuc]Dap. In some embodiments, X⁶ is 3COOHF. In some embodiments, X⁶ is Asp. In some embodiments, X⁶ is TfeGA. In some embodiments, X⁶ is Glu. In some embodiments, 3COOHF provides better properties and/or activities than, e.g., Asp.

In some embodiments, X⁶ is an amino acid residue for stapling as described herein. In some embodiments, X⁶ is stapled. In some embodiments, X⁶ is a reside of B5

In some embodiments, X⁶ is or comprises a residue of an amino acid or a moiety selected from Table A-IV.

In some embodiments, X⁶ interacts with Tyr306 of beta-catenin or an amino acid residue corresponding thereto. In some embodiments, X⁶ interacts with Lys345 of beta-catenin or an amino acid residue corresponding thereto.

Various types of amino acid residues can be used for X⁷, e.g., a residue of an amino acid of formula A-I, A-II, A-III, A-IV, A-V, A-VI, etc. or a salt thereof in accordance with the present disclosure. In some embodiments, X⁷ is —N(R^{a1})-L^{a1}-C(R^{a2})(R^{a3})-L^{a2}-C(O)—, wherein each variable is independently as described herein. In some embodiments, X⁷ is —N(R^{a1})—C(R^{a2})(R^{a3})—C(O)—, wherein each variable is independently as described herein. In some embodiments, X⁷ is —N(R^{a1})—C(R^{a2})H—C(O)—, wherein each variable is independently as described herein. In some embodiments, R^{a1} is —H. In some embodiments, R^{a3} is —H.

In some embodiments, R^{a2} is R, wherein R is C₁₋₁₀ aliphatic. In some embodiments, R^{a3} is R, wherein R is C₁₋₁₀ aliphatic. In some embodiments, each of R^{a2} and R^{a3} is independently R as described herein. In some embodiments, R^{a2} and R^{a3} are the same. In some embodiments, R is C₁₋₁₀ alkyl. In some embodiments, R is methyl.

In some embodiments, X⁷ is a residue of an amino acid whose side chain is hydrophobic. In some embodiments, X⁷ is a hydrophobic amino acid residue described herein, e.g., those described for X³. In some embodiments, X⁷ is a residue of an amino acid whose side chain is optionally substituted C₁₋₁₀ alkyl. In some embodiments, X⁷ is a residue of an amino acid whose side chain is C₁₋₁₀ alkyl. In some embodiments, X⁷ is a residue of an amino acid whose side chain is C₁₋₁₀ alkyl optionally substituted with one or more non-polar and non-charged groups. In some embodiments, X⁷ comprises a side chain comprising a cycloaliphatic group (e.g., a 3-, 4-, 5-, or 6-membered cycloalkyl group). In some embodiments, X⁷ is a residue of Aib, Ala, nLeu, Cha, Npg, sAla, Val, CyLeu, Leu, aMeL, DaMeL, or aMeV. In some embodiments, X⁷ is a residue of Aib, Ala, nLeu, or Cha. In some embodiments, X⁷ is a residue of Aib. In some embodiments, X⁷ is a residue of Ala. In some embodiments, X⁷ is a residue of nLeu. In some embodiments, X⁷ is a residue of Cha. In some embodiments, X⁷ is a residue of Npg. In some embodiments, X⁷ is a residue of sAla. In some embodiments, X⁷ is a residue of Val. In some embodiments, X⁷ is a residue of CyLeu. In some embodiments, X⁷ is a residue of Leu. In some embodiments, X⁷ is a residue of Cpg. In some embodiments, X⁷ is a residue of Cbg. In some embodiments, X⁷ is a residue of aMeL. In some embodiments, X⁷ is a residue of DaMeL. In some embodiments, X⁷ is a residue of aMeV.

In some embodiments, X⁷ is a residue of amino acid whose side chain comprises a polar group. Various polar amino acid residues described herein may be utilized for X⁷. In some embodiments, X⁷ is a residue of amino acid whose side chain comprises —OH. For example, in some embodiments, X⁷ is a residue of Ser. In some embodiments, X⁷ is a residue of Hse. In some embodiments, X⁷ is a residue of Thr. In some embodiments, X⁷ is a residue of DaMeS. In some embodiments, X⁷ is a residue of aMeS.

In some embodiments, X⁷ is a residue of amino acid that comprises an acidic or polar group. In some embodiments, X⁷ is a residue of amino acid whose side chain comprises an acidic group, e.g., a —COOH group or a salt form thereof (e.g., a compound of formula A-IV, etc.). Various acidic amino acid residues described herein may be utilized for X⁷. In some embodiments, X⁷ is a residue of 3COOHF. In some embodiments, X⁷ is a residue of amino acid whose side chain comprises an amide group, e.g., —C(O)N(R′)₂ such as —CONH₂. In some embodiments, X⁷ is a residue of Asn. In some embodiments, X⁷ is a residue of Gln. In some embodiments, X⁷ is a residue of Me2Gln. In some embodiments, X⁷ is a residue of AcLys.

In some embodiments, X⁷ comprises a side chain comprising an optionally substituted aromatic group. Various aromatic amino acid residues described herein may be utilized for X⁷. In some embodiments, an aromatic group is optionally substituted 5-membered heteroaryl having 1-3 nitrogen atoms. In some embodiments, X⁷ is a residue of Phe. In some embodiments, X⁷ is a residue of aMeDF. In some embodiments, X⁷ is a residue of aMeF. In some embodiments, X⁷ is a residue of His.

In some embodiments, X⁷ is selected from Aib, Ala, MorphGln, Gln, GlnR, Ser, iPrLys, nLeu, Cha, Hse, Lys, Npg, sAla, TriAzLys, Val, CyLeu, 3COOHF, Thr, Phe, [29N2spiroundecane]GlnR, Acp, Asn, DaMeS, aMeDF, [4aminopiperidine]GlnR, Leu, Cpg, Cbg, Me2Gln, Met20, AcLys, His, aMeL, DaMeL, aMeV, aMeS, aMeF, [isophthalate]Lys, [succinate]Lys, [Me2Mal]Lys, [diphenate]Lys, or [Biphen33COOH]Lys. In some embodiments, X⁷ is selected from GlnR, Lys, [29N2spiroundecane]GlnR, [4aminopiperidine]GlnR, sAla, TriAzLys, [isophthalate]Lys, [succinate]Lys, [Me2Mal]Lys, [diphenate]Lys, or [Biphen33COOH]Lys.

In some embodiments, X⁷ is an amino acid residue suitable for stapling as described herein.

In some embodiments, an amino acid residue suitable for stapling is —N(R^{a1})-L^{a1}-C(-L^{a}-R^{SP1})(R^{a1})-L^{a2}-C(O)— wherein each variable is independently as described herein. In some embodiments, it is —N(R^{a1})—C(-L^{a}-R^{SP1})(R^{a3})—C(O)— wherein each variable is independently as described herein. In some embodiments, in a pair of amino acid residues suitable for stapling, each amino acid residue is independently —N(R^{a1})-L^{a1}-C(-L^{a}-R^{SP1})(R^{a3})-L^{a2}-C(O)— or —N(R^{a1})—C(-L^{a}-R^{SP1})(R^{a3})—C(O)—, wherein each variable is independently as described herein. In some embodiments, R^{a1} is —H. In some embodiments, R^{a3} is —H. In some embodiments, both R^{a1} and R^{a3} are —H.

In some embodiments, R^{SP1} of a one amino acid residue in a pair is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is C₁₋₆ aliphatic. In some embodiments, R is C₁₋₆ alkyl. In some embodiments, R^{SP1} is —NH₂. In some embodiments, such an amino acid residue can be stapled with another amino acid residue comprising —COOH through amidation to form a staple comprising —C(O)N(R′)—, e.g., L^{s} wherein L^{s2} is or comprising —C(O)N(R′)—. In some embodiments, in the other amino acid residue of a pair R^{SP1} is —COOH or an active derivative thereof. In some embodiments, in the other amino acid residue of a pair R^{SP1} is —COOH. In some embodiments, R′ is R. In some embodiments, R′ is —H. In some embodiments, L^{s1} is L^{a} of a first amino acid residue, e.g., X⁷. In some embodiments, L^{s3} is L^{a} of a second amino acid residue, e.g., a C-direction amino acid residue of a first amino acid residue. In some embodiments, a first amino acid residue is X⁷, and a second amino acid residue is a C-direction amino acid residue of X⁷, e.g., X¹⁰. In some embodiments, each of L^{s1} and L^{s3} is independently L. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, each of L^{s1} and L^{s3} is independently L, wherein L is an optionally substituted bivalent linear or branched C₁₋₁₀ hydrocarbon chain. In some embodiments, each of L^{s1} and L^{s3} is independently L, wherein L is optionally substituted —(CH₂)n-, wherein n is 1-10. In some embodiments, L^{s}-(CH₂)n1-C(O)N(R′)—(CH₂)n2- wherein each variable is independently as described herein. In some embodiments, each of n1 and n2 is independently 1-10. In some embodiments, a first amino acid residue has R^{SP1} which is an amino group, and a second amino acid residue has R^{SP1} which is —COOH or an activated form thereof. In some embodiments, a second amino acid residue has R^{SP1} which is an amino group, and a first amino acid residue has R^{SP1} which is —COOH or an activated form thereof. In some embodiments, a first amino acid residue is X⁷ and a second amino acid residue is one of its C-direction amino acid residue, e.g., X¹⁰. In some embodiments, a second amino acid residue is X⁷ and a first amino acid residue is one of its N-direction amino acid residue, e.g., X³. In some embodiments, a first amino acid residue is X⁷. In some embodiments, X⁷ is Lys. In some embodiments, a second amino acid residue is X¹⁰. In some embodiments, X¹⁰ is GlnR. In some embodiments, n1 is 4 as in Lys. In some embodiments, n2 is 2 as in GlnR. In some embodiments, a first amino acid residue is X⁷, e.g., GlnR. In some embodiments, n1 is 2. In some embodiments, a second amino acid residue is X¹⁴, e.g., Lys. In some embodiments, n2 is 4. In some embodiments, a second amino acid residue is

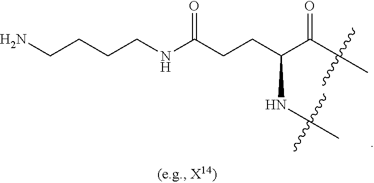 [SMILES: *N[C@@H](CCC(=O)NCCCCN)C(*)=O]

In some embodiments, L^{s3} is —(CH₂)₂—C(O)NH—(CH₂)₄—. In some embodiments, a second amino acid residue is

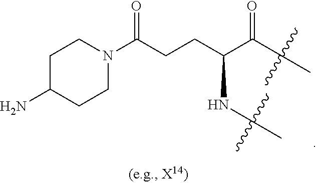 [SMILES: *N[C@@H](CCC(=O)N1CCC(N)CC1)C(*)=O]

In some embodiments, L^{s3} is —(CH₂)₂—C(O)-Cy-. In some embodiments, -Cy- is optionally substituted

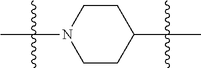 [SMILES: *C1CCN(*)CC1]

wherein the nitrogen is bonded to —C(O)—. In some embodiments, L^{s3} is —(CH₂)₂—C(O)—N(R′)—(CH₂)n-CHR′—, wherein the two R′ are taken together with their intervening atoms to form an optionally substituted ring as described herein. In some embodiments, a formed ring is optionally substituted

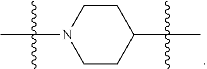 [SMILES: *C1CCN(*)CC1]

In some embodiments, a second amino acid residue is

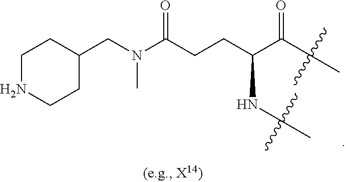 [SMILES: *N[C@@H](CCC(=O)N(C)CC1CCNCC1)C(*)=O]

In some embodiments, L^{s3} is —(CH₂)₂—C(O)—N(R′)—(CH₂)n-Cy-. In some embodiments, R′ is R as described herein. In some embodiments, R is —H. In some embodiments, R optionally substituted C₁₋₆ aliphatic. In some embodiments, R optionally substituted C₁₋₆ alkyl. In some embodiments, R is methyl. In some embodiments, n is 1. In some embodiments, -Cy- is optionally substituted

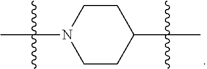 [SMILES: *C1CCN(*)CC1]

wherein the nitrogen is bonded to L^{s2} which is or comprises —C(O)—. In some embodiments, L^{s3} is —(CH₂)₂—C(O)—N(R′)—CH₂—CHR′—(CH₂)n-. In some embodiments, n is 2. In some embodiments, —(CH₂)n- is bonded to —N(R′)— of L^{s2} which is —C(O)—N(R′)—. In some embodiments, R′ of —CHR′— of L^{s3} is taken together with R′ of —N(R′)— of L^{s2} and their intervening atoms to form an optionally substituted ring as described herein. In some embodiments, a formed ring is optionally substituted

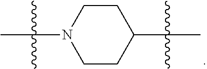 [SMILES: *C1CCN(*)CC1]

In some embodiments, a second amino acid residue is

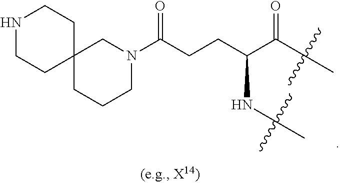 [SMILES: *N[C@@H](CCC(=O)N1CCCC2(CCNCC2)C1)C(*)=O]

In some embodiments, a second amino acid residue is

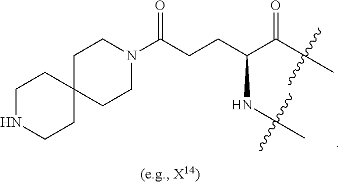 [SMILES: *N[C@@H](CCC(=O)N1CCC2(CCNCC2)CC1)C(*)=O]

In some embodiments, L^{s3}-(CH₂)₂—C(O)—N(R′)—(CH₂)ₙ₁—C(R′)₂—(CH₂)ₙ₂—. In some embodiments, each of n1 and n2 is independently 1-10. In some embodiments, n1 is 1. In some embodiments, n1 is 2. In some embodiments, n2 is 2. In some embodiments, R′ of —N(R′)— and one R′ of —C(R′)₂— are taken together with their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, L^{s2} is —C(O)N(R′)—. In some embodiments, —N(R′)— is bonded to —(CH₂)ₙ₂—. In some embodiments, one R′ of —C(R′)₂— of L^{s3} is taken together with R′ of —N(R′)— of L^{s2} and their intervening atoms to form an optionally substituted ring as described herein. In some embodiments, a formed ring is an optionally substituted 6-membered monocyclic saturated ring having no heteroatoms in addition to the nitrogen atom of —N(R′)—.

In some embodiments, a first amino acid residue is

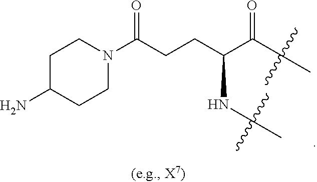 [SMILES: *N[C@@H](CCC(=O)N1CCC(N)CC1)C(*)=O]

In some embodiments, L^{s1} is —(CH₂)₂—C(O)—N(R′)—(CH₂)n-CHR′—, wherein the two R′ are taken together with their intervening atoms to form an optionally substituted ring as described herein. In some embodiments, a formed ring is optionally substituted

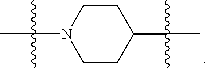 [SMILES: *C1CCN(*)CC1]

In some embodiments, a second amino acid residue is GlnR (e.g., X¹⁴). In some embodiments, L^{s3} is —(CH₂)₂—.

In some embodiments, a first amino acid residue is

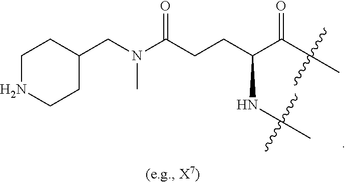 [SMILES: *N[C@@H](CCC(=O)N(C)CC1CCNCC1)C(*)=O]

In some embodiments, L^{s1} is —(CH₂)₂—C(O)—N(R′)—(CH₂)n-Cy-. In some embodiments, R′ is R as described herein. In some embodiments, R is —H. In some embodiments, R optionally substituted C₁₋₆ aliphatic. In some embodiments, R optionally substituted C₁₋₆ alkyl. In some embodiments, R is methyl. In some embodiments, n is 1. In some embodiments, -Cy- is optionally substituted

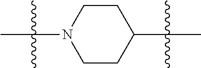 [SMILES: *C1CCN(*)CC1]

wherein the nitrogen is bonded to L^{s2} which is or comprises —C(O)—. In some embodiments, L^{s} is —(CH₂)₂—C(O)—N(R′)—CH₂—CHR′—(CH₂)n-. In some embodiments, n is 2. In some embodiments, —(CH₂)n- is bonded to —N(R′)— of L^{s2} which is —C(O)—N(R′)—. In some embodiments, R′ of —CHR′— of L^{s} is taken together with R′ of —N(R′)— of L^{s2} and their intervening atoms to form an optionally substituted ring as described herein. In some embodiments, a formed ring is optionally substituted

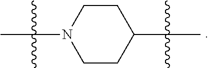 [SMILES: *C1CCN(*)CC1]

In some embodiments, a first amino acid residue is

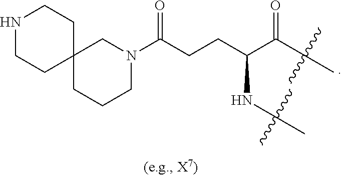 [SMILES: *N[C@@H](CCC(=O)N1CCCC2(CCNCC2)C1)C(*)=O]

In some embodiments, a first amino acid residue is

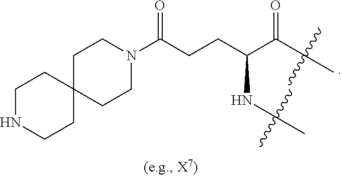 [SMILES: *N[C@@H](CCC(=O)N1CCC2(CCNCC2)CC1)C(*)=O]

In some embodiments, L^{s}-(CH₂)₂—C(O)—N(R′)—(CH₂)ₙ₁—C(R′)₂—(CH₂)ₙ₂—. In some embodiments, each of n1 and n2 is independently 1-10. In some embodiments, n1 is 1. In some embodiments, n1 is 2. In some embodiments, n2 is 2. In some embodiments, R′ of —N(R′)— and one R′ of —C(R′)₂— are taken together with their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, L^{s2} is —C(O)N(R′)—. In some embodiments, —N(R′)— is bonded to —(CH₂)ₙ₂—. In some embodiments, one R′ of —C(R′)₂— of L^{s1} is taken together with R′ of —N(R′)— of L^{s2} and their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, a second amino acid residue is GlnR (e.g., X¹⁴).

In some embodiments, a first residue is

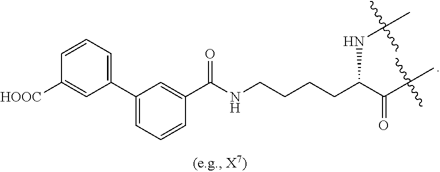 [SMILES: *N[C@@H](CCCCNC(=O)c1cccc(-c2cccc(C(=O)O)c2)c1)C(*)=O]

In some embodiments, a first residue is

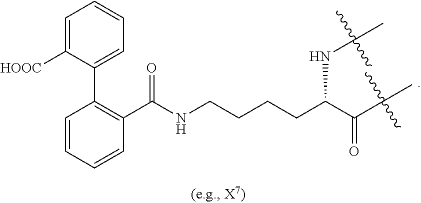 [SMILES: *N[C@@H](CCCCNC(=O)c1ccccc1-c1ccccc1C(=O)O)C(*)=O]

In some embodiments, a first residue is

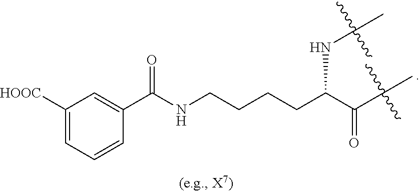 [SMILES: *N[C@@H](CCCCNC(=O)c1cccc(C(=O)O)c1)C(*)=O]

In some embodiments, L^{s1} is —(CH₂)n-N(R′)—C(O)-Cy-Cy-, wherein each variable is independently as described herein. In some embodiments, L^{s1} is —(CH₂)n-N(R′)—C(O)-Cy-, wherein each variable is independently as described herein. In some embodiments, a first residue is

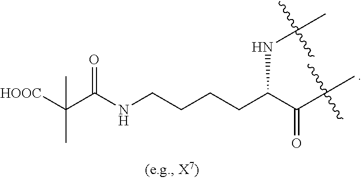 [SMILES: *N[C@@H](CCCCNC(=O)C(C)(C)C(=O)O)C(*)=O]

In some embodiments, L^{s1} is —(CH₂)n-N(R′)—C(O)—CH₂—, wherein R is as described herein, and the —CH₂— bonded to C(O)— is optionally substituted. In some embodiments, L^{s} is —(CH₂)n-N(R′)—C(O)—C(R′)₂—, wherein each R is independently as described herein. In some embodiments, L^{s1} is —(CH₂)n-N(R′)—C(O)—C(CH₃)₂—, wherein R is as described herein. In some embodiments, a first residue is

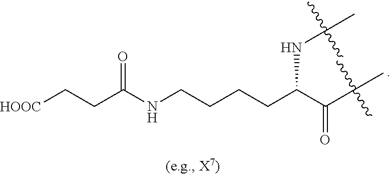 [SMILES: *N[C@@H](CCCCNC(=O)CCC(=O)O)C(*)=O]

In some embodiments, L^{s1} is —(CH₂)ₙ₁—N(R′)—C(O)—(CH₂)ₙ₂—, wherein each variable is independently as described herein. In some embodiments, each of n1 and n2 is independently n as described herein. In some embodiments, L^{s1} is —(CH₂)₄—N(R′)—C(O)—(CH₂)₂—, wherein each R is independently as described herein. In some embodiments, n is 1-10. In some embodiments, n is 1. In some embodiments, n is 2. In some embodiments, n is 3. In some embodiments, n is 4. In some embodiments, R′ is R as described herein. In some embodiments, R is —H. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s2} is or comprises —C(O)—N(R′)— as described herein. In some embodiments, R′ is R as described herein. In some embodiments, R is —H. In some embodiments, L^{s2} is —C(O)NH—. In some embodiments, —C(O)— is bonded to -Cy- of L^{s1}. In some embodiments, a second residue is X¹⁴, e.g., Lys. In some embodiments, L^{s3} is as described herein, e.g., optionally substituted —(CH₂)n-. In some embodiments, L^{s3} is —(CH₂)n-. In some embodiments, n is 1. In some embodiments, n is 2. In some embodiments, n is 3. In some embodiments, n is 4 (e.g., as in Lys).

In some embodiments, R^{SP1} of a first amino acid residue is or comprises —COOH or a protected or activated form thereof. In some embodiments, a first amino acid residue is X³, e.g., GlnR. In some embodiments, R^{SP1} of a second amino acid residue is or comprises an amino group, e.g., —NHR as described herein. In some embodiments, R^{SP1} of a second amino acid residue is or comprises —NH₂. In some embodiments, a second amino acid residue is X⁷, e.g., Lys. In some embodiments, each of L^{s1} and L^{s3} is independently optionally substituted —(CH₂)n-, wherein n is 1-10. In some embodiments, L^{s1} is —(CH₂)₂—. In some embodiments, L^{s1} is —(CH₂)₄—.

In some embodiments, R^{SP1} of a one amino acid residue in a pair is a first reaction group of a cycloaddition reaction. In some embodiments, such an amino acid residue can be stapled with another amino acid residue comprising a second reactive group of a cycloaddition reaction through a cycloaddition reaction. In some embodiments, in the other amino acid residue of a pair R^{SP1} is a second reactive group of a cycloaddition reaction. In some embodiments, a cycloaddition reaction is [3+2]. In some embodiments, a cycloaddition reaction is a click chemistry reaction. In some embodiments, a cycloaddition reaction is [4+2]. In some embodiments, one of the first and the second reactive groups is or comprises —N₃, and the other is or comprises an alkyne (e.g., a terminal alkyne or activated/strained alkyne).

In some embodiments, R^{SP1} of a first amino acid residue is —N₃. In some embodiments, L^{a} of a first amino acid residue is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, R^{SP1} of a second amino acid residue is or comprises —C≡C—. In some embodiments, R^{SP1} of a second amino acid residue is —≡—H. In some embodiments, R^{SP1} of a second amino acid residue comprises a strained alkyne, e.g., in a ring. In some embodiments, L^{a} of a first amino acid residue is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, L^{s} is -L^{s1}-L^{s2}-L^{s3}-, wherein L^{s2} is or comprises -Cy-. In some embodiments, L^{s2} is -Cy-. In some embodiments, -Cy- is formed by a cycloaddition reaction. In some embodiments, -Cy- is optionally substituted

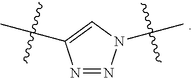 [SMILES: *c1cn(*)nn1]

In some embodiments, -Cy- is

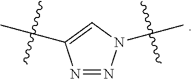 [SMILES: *c1cn(*)nn1]

In some embodiments, -Cy- is formed by a cycloaddition reaction. In some embodiments, -Cy- is optionally substituted

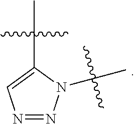 [SMILES: *c1cnnn1*]

In some embodiments, -Cy- is

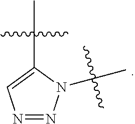 [SMILES: *c1cnnn1*]

In some embodiments, L^{s1} is L^{a} of a first amino acid residue, and L^{s3} is L^{a} of a second amino acid residue. In some embodiments, L^{s1} is L^{a} of a second amino acid residue, and L^{s3} is L^{a} of a first amino acid residue. In some embodiments, each of L^{s1} and L^{s3} is independently L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s1} is optionally substituted —(CH₂)n-, wherein n is 1-10. In some embodiments, L^{s1} is —(CH₂)n-, wherein n is 1-10. In some embodiments, n is 1. In some embodiments, n is 2. In some embodiments, n is 3. In some embodiments, n is 4. In some embodiments, L^{s3} is optionally substituted —(CH₂)n-, wherein n is 1-10. In some embodiments, L^{s3} is —(CH₂)n-, wherein n is 1-10. In some embodiments, n is 1. In some embodiments, n is 2. In some embodiments, n is 3. In some embodiments, n is 4.

In some embodiments, a first amino acid residue is X⁷. In some embodiments, R^{SP1} of X⁷ is —N₃. In some embodiments, L^{a} of X⁷ is optionally substituted —(CH₂)n- wherein n is 1-10. In some embodiments, L^{a} of X⁷ is —(CH₂)₄—. In some embodiments, L^{a} of X⁷ is —(CH₂)₃—. In some embodiments, L^{a} of X⁷ is —(CH₂)₂—. In some embodiments, L^{a} of X⁷ is —CH₂—. In some embodiments, a second amino acid residue is X¹⁰. In some embodiments, R^{SP1} of X¹⁰ is or comprises an alkyne, e.g., a strained/activated alkyne. In some embodiments, R^{SP1} of X¹⁰ is —C≡CH. In some embodiments, L^{a} of X¹⁰ is optionally substituted —(CH₂)n- wherein n is 1-10. In some embodiments, L^{a} of X¹⁰ is —(CH₂)₄—. In some embodiments, L^{a} of X¹⁰ is —(CH₂)₃—. In some embodiments, L^{a} of X¹⁰ is —(CH₂)₂—. In some embodiments, L^{a} of X¹⁰ is —CH₂—. In some embodiments, L^{s3} is L^{a} of X¹⁰. In some embodiments, L^{s3} is bonded to a carbon atom of L^{s2}.

In some embodiments, a first amino acid residue is X⁷. In some embodiments, R^{SP1} of X⁷ is or comprises an alkyne, e.g., a strained/activated alkyne. In some embodiments, R^{SP1} of X⁷ is —C≡CH. In some embodiments, L^{a} of X⁷ is optionally substituted —(CH₂)n- wherein n is 1-10. In some embodiments, L^{a} of X⁷ is —(CH₂)₄—. In some embodiments, L^{a} of X⁷ is —(CH₂)₃—. In some embodiments, L^{a} of X⁷ is —(CH₂)₂—. In some embodiments, L^{a} of X⁷ is —CH₂—. In some embodiments, L^{s1} is L^{a} of X⁷. In some embodiments, L^{s1} is bonded to a carbon atom of L^{s2}.In some embodiments, a second amino acid residue is X¹⁰. In some embodiments, R^{SP1} of X¹⁰ is —N₃. In some embodiments, L^{a} of X¹⁰ is optionally substituted —(CH₂)n- wherein n is 1-10. In some embodiments, L^{a} of X¹⁰ is —(CH₂)₄—. In some embodiments, L^{a} of X¹⁰ is —(CH₂)₃—. In some embodiments, L^{a} of X¹⁰ is —(CH₂)₂—. In some embodiments, L^{a} of X¹⁰ is —CH₂—.

In some embodiments, R^{SP1} of two amino acid residues of a pair of amino acid residues suitable for stapling can each independently react with a linking reagent to form a staple. In some embodiments, a suitable linking reagent comprises two reactive groups, each can independently react with R^{SP1} of each amino acid residue. In some embodiments, a linking reagent has the structure of H-L″-H or a salt thereof, wherein the reagent comprises two amino groups, and L^{s1} is a covalent bond, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, such a linking agent can react with two amino acid residues each independently having a R^{SP1} group that is —COOH or an activated form thereof.

Suitable embodiments for L″ including those described for L herein that fall within the scope of L″. For example, in some embodiments, L^{s1} is L wherein L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, a linking reagent is a diamine or a salt thereof. In some embodiments, a reagent has the structure of NHR-L″-NHR or a salt thereof, wherein each variable is independently as described herein. In some embodiments, each R is independently —H or optionally substituted C₁₋₆ aliphatic. In some embodiments, each R is independently —H or C₁₋₆ aliphatic. In some embodiments, each R is independently —H or optionally substituted C₁₋₆ alkyl. In some embodiments, each R is independently —H or C₁₋₆ alkyl. In some embodiments, a reagent has the structure of NH₂-L″-NH₂ or a salt thereof. In some embodiments, L^{s1} is optionally substituted —(CH₂)n- wherein n is 1-10. In some embodiments, L″ is —(CH₂)₄—.

In some embodiments, a staple, L^{s}, is -L^{s1}-L^{s2}-L^{s3}-, wherein L^{s1} is L^{a} of a first amino acid residue of a stapled pair, L^{s3} is L^{a} of a second amino acid residue of a stapled pair, and L^{s2} is —C(O)—N(R′)-L″-N(R′)—C(O)—, wherein each variable is independently as described herein. In some embodiments, L^{s1} is optionally substituted —(CH₂)n- wherein n is 1-10. In some embodiments, L″ is —(CH₂)₄—. In some embodiments, each of L^{s1} and L^{s3} is independently optionally substituted —(CH₂)n- wherein n is 1-10. In some embodiments, n is 2. In some embodiments, a first amino acid residue is Gln (e.g., X⁷). In some embodiments, a second amino acid residue is GlnR (e.g., X¹⁴). In some embodiments, two GlnR can form such a staple through [diaminobutane].

In some embodiments, a linking reagent has the structure of H-Cy-L″-NHR or a salt thereof, wherein -Cy- comprises a second amino group. In some embodiments, R is —H or optionally substituted C₁₋₆ aliphatic. In some embodiments, R is —H or C₁₋₆ aliphatic. In some embodiments, R is —H or optionally substituted C₁₋₆ alkyl. In some embodiments, R is —H or C₁₋₆ alkyl. In some embodiments, R is methyl. In some embodiments, a linking reagent has the structure of H-Cy-L″-NH₂ or a salt thereof, wherein -Cy-comprises a second amino group. In some embodiments -Cy- is optionally substituted

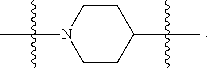 [SMILES: *C1CCN(*)CC1]

In some embodiments, -Cy- is

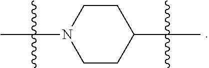 [SMILES: *C1CCN(*)CC1]

In some embodiments, L^{s1} is a covalent bond. In some embodiments, L^{s1} is optionally substituted —(CH₂)n- wherein n is 1-10. In some embodiments, L″ is —(CH₂)—. In some embodiments, a linking reagent is

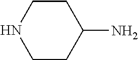

or a salt thereof. In some embodiments, a linking reagent is

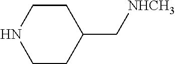

or a salt thereof.
as described herein. In some embodiments, R′ is —H. In some embodiments, -Cy- is

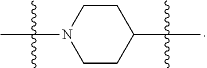 [SMILES: *C1CCN(*)CC1]

In some embodiments, each of L^{s1} and L^{s3} is independently optionally substituted —(CH₂)n- wherein n is 1-10. In some embodiments, n is 2. In some embodiments, -Cy- is closer to a N-terminus than —N(R′)—. In some embodiments, -Cy- is closer to a C-terminus than —N(R′)—. In some embodiments, a first amino acid residue is Gln (e.g., X⁷). In some embodiments, a second amino acid residue is GlnR (e.g., X¹⁴). In some embodiments, two GlnR can form such a staple through [4aminopiperidine].

In some embodiments, L^{s2} is —C(O)-Cy-(CH₂)n-N(R′)—C(O)—, wherein each variable is independently as described herein. In some embodiments, R′ is —H. In some embodiments, R′ is R as described herein, e.g., optionally substituted C₁₋₆ aliphatic, C₁₋₆ alkyl, etc. In some embodiments, R is methyl. In some embodiments, n is 1. In some embodiments, -Cy- is

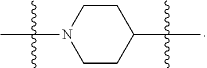 [SMILES: *C1CCN(*)CC1]

In some embodiments, -Cy- is closer to a N-terminus than —N(R′)—. In some embodiments, -Cy- is closer to a C-terminus than —N(R′)—. In some embodiments, each of L^{s1} and L^{s3} is independently optionally substituted —(CH₂)n- wherein n is 1-10. In some embodiments, n is 2. In some embodiments, a first amino acid residue is Gln (e.g., X⁷). In some embodiments, a second amino acid residue is GlnR (e.g., X¹⁴). In some embodiments, two GlnR can form such a staple through [4mampiperidine].

In some embodiments, a methylene unit is replaced with -Cy-. In some embodiments, a linking reagent has the structure of H-Cy-H, wherein Cy comprises two secondary amino groups. In some embodiments, -Cy- is optionally substituted 8-20 membered bicyclic ring. In some embodiments, H-Cy-H comprises two —NH—. In some embodiments, -Cy- is optionally substituted

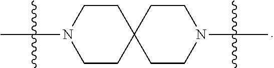 [SMILES: *N1CCC2(CC1)CCN(*)CC2]

In some embodiments, -Cy- is optionally substituted

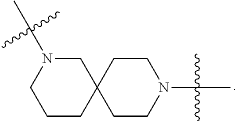 [SMILES: *N1CCC2(CCCN(*)C2)CC1]

In some embodiments, the meta connection site (relative to the spiro carbon atom) is closer to a N-terminus than the para connection site (relative to the spiro carbon atom). In some embodiments, the meta connection site (relative to the spiro carbon atom) is closer to a C-terminus than the para connection site (relative to the spiro carbon atom).

In some embodiments, L^{s2} is —C(O)-Cy-C(O)— wherein -Cy- is as described herein. In some embodiments, each of L^{s1} and L^{s3} is independently optionally substituted —(CH₂)n- wherein n is 1-10. In some embodiments, n is 2. In some embodiments, a first amino acid residue is Gln (e.g., X⁷). In some embodiments, a second amino acid residue is GlnR (e.g., X¹⁴). In some embodiments, two GlnR can form such a staple through [29N2spiroundecane]. In some embodiments, two GlnR can form such a staple through [39N2spiroundecane].

In some embodiments, a pair of amino acid residue suitable for stapling both independently has the structure of —N(R^{a1})-L^{a1}-C-L^{a}-R^{SP1})(R^{a3})-L^{a2}-C(O)— or —N(R^{a1})—C(-L^{a}-R^{SP1})(R^{a3})—C(O)—, wherein each variable is independently as described herein, and R^{SP1} is an amino group. In some embodiments, R^{SP1} is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is C₁₋₆ aliphatic. In some embodiments, R is C₁₋₆ alkyl. In some embodiments, R^{SP1} is —NH₂. In some embodiments, such two amino acid residue may be linked by a di-acid linking reagent.

In some embodiments, a linking reagent has the structure of HOOC-L″-COOH, or a salt thereof, or an activated form thereof, wherein L^{s1} is as described herein. In some embodiments, L^{s1} is -Cy-Cy-. In some embodiments, L^{s1} is -Cy-. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s1} is optionally substituted

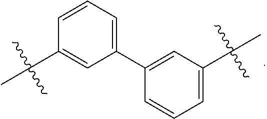 [SMILES: *c1cccc(-c2cccc(*)c2)c1]

In some embodiments, a linking agent is

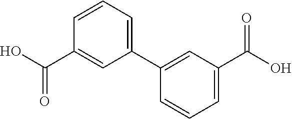 [SMILES: O=C(O)c1cccc(-c2cccc(C(=O)O)c2)c1]

or a salt or an activated form thereof. In some embodiments, L″ is optionally substituted

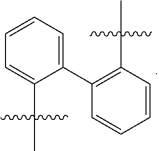 [SMILES: *c1ccccc1-c1ccccc1*]

In some embodiments, a linking agent is

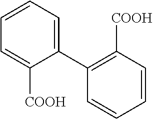 [SMILES: O=C(O)c1ccccc1-c1ccccc1C(=O)O]

or a salt or an activated form thereof. In some embodiments, L″ is 1,3-phenylene. In some embodiments, a linking agent is

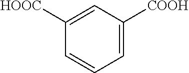 [SMILES: O=C(O)c1cccc(C(=O)O)c1]

or a salt or an activated form thereof. In some embodiments, L″ is optionally substituted —(CH₂)n-, wherein n is 1-10. In some embodiments, L″ is optionally substituted —CH₂—. In some embodiments, L″ is —C(R′)₂—. In some embodiments, L″ is —C(CH₃)₂—. In some embodiments, a linking agent is (CH₃)₂C(COOH)₂ or a salt or an activated form thereof. In some embodiments, L″ is —CH₂CH₂—. In some embodiments, a linking agent is HOOCCH₂CH₂COOH or a salt or an activated form thereof.

In some embodiments, a staple is L^{s}, wherein L^{s2} is —N(R′)-L″-N(R′)—, and each of L^{s1} and L^{s3} is independently as described herein. In some embodiments, L^{s1} is -Cy-Cy-, wherein each -Cy- is independently as described herein. In some embodiments, L^{s1} is -Cy- as described herein. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s1} is optionally substituted —(CH₂)n-, wherein n is 1-10. In some embodiments, L^{s1} is optionally substituted —CH₂—. In some embodiments, L^{s1} is —C(R′)₂—. In some embodiments, L^{s1} is —C(CH₃)₂—. In some embodiments, L″ is —CH₂CH₂—. In some embodiments, each of L^{s1} and L^{s3} is independently optionally substituted —(CH₂)n- wherein n is 1-10. In some embodiments, n is 2. In some embodiments, n is 4. In some embodiments, a first amino acid residue is Lys (e.g., X⁷). In some embodiments, a second amino acid residue is Lys (e.g., X¹⁴). In some embodiments, two Lys can form such a staple through [Biphen33COOH]. In some embodiments, two Lys can form such a staple through [diphenate]. In some embodiments, two Lys can form such a staple through [isophthalate]. In some embodiments, two Lys can form such a staple through [Me2Mal]. In some embodiments, two Lys can form such a staple through [succinate].

In some embodiments, X⁷ is stapled. In some embodiments, X⁷ is stapled with X¹⁴. In some embodiments, X⁷ is stapled with X¹⁰. In some embodiments, X¹⁰ is stapled with X⁷. In some embodiments, X⁷ is stapled with X³.

In some embodiments, X⁷ is Aib, Ala, 3COOHF, CyLeu, Phe, Asp, nLeu, B5, Val, Gln, MorphGln, GlnR, Cha, Ser, Leu, Cbg, CyhLeu, iPrLys, Aic, Lys, Lys*, Hse, GlnR, Npg, GlnR*, Dpg, Gly, sAla, TriAzLys, Thr, Asn, dAla, [isophthalate]-Lys, [succinate]-Lys, [29N2spiroundecane]GlnR, Acp, DaMeS, aMeDF, DGlnR, [Ac] Acp, [Phc] Acp, [isovaleryl]Acp, [Me2Mal]-Lys, [diphenate]-Lys, [Biphen33COOH]-Lys, [Me2Mal]Lys, [diphenate]Lys, [Biphen33COOH]Lys, [4aminopiperidine]GlnR, Cpg, Me2Gln, Met20, AcLys, His, aMeL, DaMeL, aMeV, aMeS, aMeF, dLys, [ethylenediamine]GlnR, [Me2ethylenediamine]GlnR, [diaminopropane]GlnR, [diaminopentane]GlnR, [Me2diaminohexane]GlnR, [Ac] PyrSa, [Phc] PyrSa, [isovaleryl]PyrSa, [Ac] PyrRa, [Phc] PyrRa, [isovaleryl]PyrRa, 2COOHF, 4COOHF, or Glu. In some embodiments, X⁷ is Aib. In some embodiments, X⁷ is Ala. In some embodiments, X⁷ is 3COOHF. In some embodiments, X⁷ is CyLeu. In some embodiments, X⁷ is Phe. In some embodiments, X⁷ is nLeu. In some embodiments, X⁷ is Val. In some embodiments, X⁷ is Cha. In some embodiments, X⁷ is Leu. In some embodiments, X⁷ is Cbg. In some embodiments, X⁷ is CyhLeu. In some embodiments, Aib provides better properties and/or activities than, e.g., Ala. In some embodiments, X⁷ is GlnPDA*3. In some embodiments, X⁷ is GlnBDA*3. In some embodiments, X⁷ is GlnR*3. In some embodiments, X⁷ is GlnMeBDA*3. In some embodiments, X⁷ is GlnT4CyMe*3. In some embodiments, X⁷ is GlnC4CyMe*3. In some embodiments, X⁷ is Gln3ACPip*3. In some embodiments, X⁷ is GlnPipAz*3. In some embodiments, X⁷ is Gln4Pippip*3. In some embodiments, X⁷ is GlnPip4AE*3.

In some embodiments, X⁷ is or comprises a residue of an amino acid or a moiety selected from Table A-I, Table A-II, Table A-III and Table A-IV.

Various types of amino acid residues can be used for X⁸, e.g., a residue of an amino acid of formula A-I, A-II, A-III, A-IV, A-V, A-VI, etc. or a salt thereof in accordance with the present disclosure. In some embodiments, X¹¹ is —N(R^{a1})-L^{a1}-C(R^{a2})(R^{a3})-L^{a2}-C(O)—, wherein each variable is independently as described herein. In some embodiments, X¹¹ is —N(R^{a1})—C(R^{a2})(R^{a3})—C(O)—, wherein each variable is independently as described herein. In some embodiments, X¹¹ is —N(R^{a1})—C(R^{a2})H—C(O)—, wherein each variable is independently as described herein. In some embodiments, R^{a1} is —H. In some embodiments, R^{a3} is —H.

In some embodiments, X⁸ is a residue of an amino acid whose side chain is hydrophobic. In some embodiments, X¹ is a hydrophobic amino acid residue as described herein, e.g., those described for X³. In some embodiments, X¹¹ is a residue of Ala. In some embodiments, X¹¹ is a residue of Aib. In some embodiments, X¹¹ is a residue of Cpg. In some embodiments, X¹¹ is a residue of Val. In some embodiments, X⁸ is a residue of Leu. In some embodiments, X¹¹ is a residue of nLeu. In some embodiments, X¹¹ is a residue of Cba.

In some embodiments, X¹¹ is a residue of amino acid that comprises an acidic or polar group. In some embodiments, X¹¹ is a residue of amino acid whose side chain comprises a polar group. In some embodiments, X¹ is a polar amino acid residue as described herein. In some embodiments, X¹ is a residue of amino acid whose side chain comprises —OH. In some embodiments, X⁸ comprises a side chain comprising an optionally substituted aromatic group. For example, in some embodiments, X¹ is a residue of Ser. In some embodiments, X¹ is a residue of Thr. In some embodiments, X¹ is a residue of aThr. In some embodiments, X¹ is a residue of hTyr. In some embodiments, X¹ is a residue of amino acid whose side chain comprises an amide group, e.g., —C(O)N(R′)₂ such as —CONH₂. In some embodiments, X¹¹ is a residue of Gln. In some embodiments, X¹¹ is a residue of AcLys.

In some embodiments, X⁸ is a residue of amino acid whose side chain comprises an acidic group, e.g., a —COOH group or a salt form thereof (e.g., a compound of formula A-IV, etc.). In some embodiments, X⁸ is an acidic amino acid residue as described herein, e.g., those descried for X², X⁵, X⁶, etc. In some embodiments, X⁸ is a residue of Asp. In some embodiments, X⁸ is a residue of Glu. In some embodiments, Xx is a residue of Aad.

In some embodiments, X⁸ comprises a side chain comprising an optionally substituted aromatic group. In some embodiments, X⁸ is an aromatic amino acid residue as described herein. In some embodiments, an aromatic group is phenyl. In some embodiments, X⁸ is a residue of Phe. In some embodiments, X⁸ is a residue of hPhe. In some embodiments, X⁸ is a residue of hTyr.

In some embodiments, X⁸ is selected from Ala, Aib, Cpg, Val, Leu, Gln, Lys, Asp, Glu, Aad, nLeu, Cba, Ser, Thr, aThr, MorphGln, Phe, hPhe, hTyr, and AcLys.

In some embodiments, Xx is Ala, Aib, Phe, Asp, 3COOHF, aThr, Gly, Ser, nLeu, Thr, Cpg, Val, Leu, Gln, Lys, Glu, Aad, Cba, MorphGln, hPhe, hTyr, or AcLys. In some embodiments, Xx is Ala. In some embodiments, Xx is Aib. In some embodiments, Xx is Phe. In some embodiments, Xx is Asp. In some embodiments, Xx is 3COOHF.

In some embodiments, Xx is or comprises a residue of an amino acid or a moiety selected from Table A-IV.

In some embodiments, X⁸ interacts with Trp383 of beta-catenin or an amino acid residue corresponding thereto.

Various types of amino acid residues can be used for X⁹, e.g., a residue of an amino acid of formula A-I, A-II, A-III, A-IV, A-V, A-VI, etc. or a salt thereof in accordance with the present disclosure. In some embodiments, X⁹ is —N(R^{a1})-L^{a1}-C(R^{a2})(R^{a3})-L^{a2}-C(O)—, wherein each variable is independently as described herein. In some embodiments, X⁹ is —N(R^{a1})—C(R^{a2})(R^{a3})—C(O)—, wherein each variable is independently as described herein. In some embodiments, X⁹ is —N(R^{a1})—C(R^{a2})H—C(O)—, wherein each variable is independently as described herein. In some embodiments, R^{a1} is —H. In some embodiments, R^{a3} is —H.

In some embodiments, X⁹ comprises a side chain comprising an optionally substituted aromatic group. In some embodiments, X⁹ is an aromatic amino acid residue as described herein. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one sulfur atom. In some embodiments, an aromatic group is optionally substituted phenyl. In some embodiments, X⁹ comprises a side chain which is or comprises an optionally substituted aromatic group, wherein each substituent of the aromatic group is independently selected from halogen, —OR, —R, —C(O)OH, or —CN, wherein each R is independently hydrogen or C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X⁹ comprises a side chain which is or comprises an optionally substituted aromatic group, wherein each substituent of the aromatic group is independently halogen. In some embodiments, X⁹ comprises a side chain which is or comprises two optionally substituted aromatic groups. In some embodiments, X⁹ comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X⁹ is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5-6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, X⁹ is a residue of an amino acid selected from Phe, 3COOHF, 2NapA, Tyr, 3Thi, 4FF, 4ClF, 4BrF, 3FF, 3ClF, 3BrF, 2FF, 30MeF, 4CNF, 3CNF, 4MeF, 3MeF, Aic, RbiPrF, SbiPrF, RbiPrDF, RbMeXylA, RbMeXylDA, BztA, 1NapA, Trp, 2Thi, 4TriA, 3F3MeF, His, SbMeXylA, and SbMeXylDA. In some embodiments, X⁹ is Phe. In some embodiments, X⁹ is 3COOHF. In some embodiments, X⁹ is 2NapA. In some embodiments, X⁹ is Tyr. In some embodiments, X⁹ is 3Thi. In some embodiments, X⁹ is 4FF. In some embodiments, X⁹ is 4ClF. In some embodiments, X⁹ is 4BrF. In some embodiments, X⁹ is 3FF. In some embodiments, X⁹ is 3ClF. In some embodiments, X⁹ is 3BrF. In some embodiments, X⁹ is 2FF. In some embodiments, X⁹ is 30MeF. In some embodiments, X⁹ is 4CNF. In some embodiments, X⁹ is 3CNF. In some embodiments, X⁹ is 4MeF. In some embodiments, X⁹ is 3MeF. In some embodiments, X⁹ is Aic. In some embodiments, X⁹ is RbiPrF. In some embodiments, X⁹ is SbiPrF. In some embodiments, X⁹ is RbiPrDF. In some embodiments, X⁹ is RbMeXylA. In some embodiments, X⁹ is RbMeXylDA. In some embodiments, X⁹ is BztA. In some embodiments, X⁹ is 1NapA. In some embodiments, X⁹ is Trp. In some embodiments, X⁹ is 2Thi. In some embodiments, X⁹ is 4TriA. In some embodiments, X⁹ is 3F3MeF. In some embodiments, X⁹ is His. In some embodiments, X⁹ is SbMeXylA. In some embodiments, X⁹ is SbMeXylDA.

In some embodiments, X⁹ is a residue of an amino acid whose side chain is hydrophobic. In some embodiments, X⁹ is a hydrophobic amino acid residue as described herein. In some embodiments, X⁹ is selected from nLeu, Ala, Cba, CypA, Leu, Ile, Chg, Val, and 2Cpg.

In some embodiments, X⁹ is a residue of amino acid that comprises an acidic or polar group. In some embodiments, X⁹ is a residue of amino acid whose side chain comprises a polar group. In some embodiments, X⁹ is a polar amino acid residue as described herein. In some embodiments, X⁹ is a residue of amino acid whose side chain comprises —OH. For example, in some embodiments, X⁹ is a residue of Ser. In some embodiments, X⁹ is a residue of Hse. In some embodiments, X⁹ is a residue of amino acid whose side chain comprises an amide group, e.g., —C(O)N(R′)₂ such as —CONH₂. For example, in some embodiments, X⁹ is a residue of Asn. In some embodiments, X⁹ is Gln.

In some embodiments, X⁹ is Phe, Ala, Lys, 3COOHF, Aib, 2NapA, nLeu, 2Thi, Tyr, 3Thi, 4FF, 4ClF, 4BrF, 3FF, 3ClF, 3BrF, 2FF, 30MeF, 4CNF, 3CNF, 4MeF, 3MeF, Aic, RbiPrF, SbiPrF, RbiPrDF, RbMeXylA, RbMeXylDA, Cba, CypA, BztA, 1NapA, Trp, Leu, Ile, Ser, Chg, Hse, 4TriA, 3F3MeF, Thr, His, Val, Asn, Gln, 2Cpg, SbMeXylA, or SbMeXylDA. In some embodiments, X⁹ is Phe. In some embodiments, X⁹ is Ala.

In some embodiments, X⁹ is or comprises a residue of an amino acid or a moiety selected from Table A-IV.

In some embodiments, X⁹ interacts with Lys345 of beta-catenin or an amino acid residue corresponding thereto. In some embodiments, X⁹ interacts with Trp383 of beta-catenin or an amino acid residue corresponding thereto. In some embodiments, X⁹ interacts with Lys345 and Trp383 of beta-catenin or amino acid residues corresponding thereto.

Various types of amino acid residues can be used for X¹⁰, e.g., a residue of an amino acid of formula A-I, A-II, A-III, A-IV, A-V, A-VI, etc. or a salt thereof in accordance with the present disclosure. In some embodiments, X¹⁰ is —N(R^{a1})-L^{a1}-C(R^{a2})(R^{a3})-L^{a2}-C(O)—, wherein each variable is independently as described herein. In some embodiments, X¹⁰ is —N(R^{a1})—C(R^{a2})(R^{a3})—C(O)—, wherein each variable is independently as described herein. In some embodiments, X¹⁰ is —N(R^{a1})—C(R^{a2})H—C(O)—, wherein each variable is independently as described herein. In some embodiments, R^{a1} is —H. In some embodiments, R^{a3} is —H.

In some embodiments, X¹⁰ is Lys, GlnR, TriAzLys, sAla, dLys, AsnR, hGlnR, iPrLys, TriAzOrn, DGlnR, Orn, 4PipA, sCH2S, [8FBB]Cys, [mXyl]Cys, [oXyl]Cys, [pXyl]Cys, dOm, dDab, NMeOm, [2-6-naph]Cys, or [3-3-biph]Cys. In some embodiments, X¹⁰ is Lys, GlnR, or TriAzLys. In some embodiments, X¹⁰ is Lys. In some embodiments, X¹⁰ is Gln. In some embodiments, X¹⁰ is TriAzLys. In some embodiments, X¹⁰ is sAla. In some embodiments, X¹⁰ is dLys. In some embodiments, X¹⁰ is AsnR. In some embodiments, X¹⁰ is hGlnR. In some embodiments, X¹⁰ is iPrLys. In some embodiments, X¹⁰ is TriAzOm. In some embodiments, X¹⁰ is DGlnR. In some embodiments, X¹⁰ is Orn. In some embodiments, X¹⁰ is 4PipA. In some embodiments, X¹⁰ is sCH2S. In some embodiments, X¹⁰ is [8FBB]Cys. In some embodiments, X¹⁰ is [4FB]Cys. In some embodiments, X¹⁰ is [mXyl]Cys. In some embodiments, X¹⁰ is [oXyl]Cys. In some embodiments, X¹⁰ is [pXyl]Cys. In some embodiments, X¹⁰ is dOm. In some embodiments, X¹⁰ is dDab. In some embodiments, X¹⁰ is NMeOm. In some embodiments, X¹⁰ is [2-6-naph]Cys. In some embodiments, X¹⁰ is [3-3-biph]Cys.

In some embodiments, X¹⁰ is not stapled (e.g., when other residues are optionally stapled). In some embodiments, X¹⁰ is a residue of Leu or Phe. In some embodiments, X¹⁰ is a residue of Leu. In some embodiments, X¹⁰ is a residue of Phe.

In some embodiments, X¹⁰ is an amino acid residue suitable for stapling as described herein.

In some embodiments, an amino acid residue suitable for stapling is —N(R^{a1})-L^{a1}-C(-L^{a}-R^{SP1})(R^{a3})-L^{a2}-C(O)— wherein each variable is independently as described herein. In some embodiments, it is —N(R^{a1})—C(-L^{a}-R^{SP1})(R^{a3})—C(O)— wherein each variable is independently as described herein. In some embodiments, in a pair of amino acid residues suitable for stapling, each amino acid residue is independently —N(R^{a1})-L^{a1}-C(-L^{a}-R^{SP1})(R^{a3})-L^{a2}-C(O)— or —N(R^{a1})—C(-L^{a}-R^{SP1})(R^{a3})—C(O)—, wherein each variable is independently as described herein. In some embodiments, R^{a1} is —H. In some embodiments, R^{a3} is —H. In some embodiments, both R^{a1} and R^{a3} are —H.

In some embodiments, R^{SP1} of a one amino acid residue in a pair is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is C₁₋₆ aliphatic. In some embodiments, R is C₁₋₆ alkyl. In some embodiments, R^{SP1} is —NH₂. In some embodiments, such an amino acid residue can be stapled with another amino acid residue comprising —COOH through amidation to form a staple comprising —C(O)N(R′)—, e.g., L^{s} wherein L^{s2} is or comprising —C(O)N(R′)—. In some embodiments, L^{s2} is —C(O)N(R′)— wherein R′ is as described herein. In some embodiments, R′ is R as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R is ethyl. In some embodiments, R is isopropyl. In some embodiments, —N(R′)— is from an amino acid residue which before stapling comprises an amino group. In some embodiments, —C(O)— is from an amino acid residue which before stapling comprises —COOH or an activated form thereof. In some embodiments, in the other amino acid residue of a pair R^{SP1} is —COOH or an active derivative thereof. In some embodiments, in the other amino acid residue of a pair R^{SP1} is —COOH. In some embodiments, R′ is R. In some embodiments, R′ is —H. In some embodiments, L^{s1} is L^{a} of a first amino acid residue, e.g., X¹⁰. In some embodiments, L^{s3} is L^{a} of a second amino acid residue, e.g., a C-direction amino acid residue of a first amino acid residue. In some embodiments, a first amino acid residue is X¹⁰, and a second amino acid residue is a C-direction amino acid residue of X¹⁰, e.g., X⁴. In some embodiments, each of L^{s1} and L^{s3} is independently L. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, each of L^{s1} and L^{s3} is independently L, wherein L is an optionally substituted bivalent linear or branched C₁₋₁₀ hydrocarbon chain. In some embodiments, each of L^{s1} and L^{s3} is independently L, wherein L is optionally substituted —(CH₂)n-, wherein n is 1-10. In some embodiments, L^{s}-(CH₂)n1-C(O)N(R′)-L^{s3}- wherein each variable is independently as described herein. In some embodiments, L^{s}-L^{s1}-C(O)N(R′)—(CH₂)n2- wherein each variable is independently as described herein. In some embodiments, L^{s}-(CH₂)n1-C(O)N(R′)—(CH₂)n2- wherein each variable is independently as described herein. In some embodiments, each of n1 and n2 is independently 1-10. In some embodiments, a first amino acid residue has R^{SP1} which is an amino group, and a second amino acid residue has R^{SP1} which is —COOH or an activated form thereof. In some embodiments, a second amino acid residue has R^{SP1} which is an amino group, and a first amino acid residue has R^{SP1} which is —COOH or an activated form thereof.

In some embodiments, a first amino acid residue is X¹⁰ and a second amino acid residue is one of its C-direction amino acid residue, e.g., X¹⁴. In some embodiments, a second amino acid residue is X¹⁰ and a first amino acid residue is one of its N-direction amino acid residue, e.g., X⁷.

In some embodiments, a first amino acid residue is X¹⁰. In some embodiments, X¹⁰ is Lys. In some embodiments, X¹⁰ is dLys. In some embodiments, X¹⁰ is iPrLys. In some embodiments, X¹⁰ is NMeOm. In some embodiments, R′ of —N(R′)— of L^{s2} is optionally substituted C₁₋₆ alkyl. In some embodiments, it is methyl. In some embodiments, it is isopropyl. In some embodiments, n1 is 4. In some embodiments, n1 is 3. In some embodiments, X¹⁰ is Orn. In some embodiments, X¹⁰ is dOm. In some embodiments, n1 is 3. In some embodiments, X¹⁰ is dDab. In some embodiments, n1 is 2. In some embodiments, —N(R′)— of L^{s2} is bonded L^{s1}. In some embodiments, a second amino acid residue is X¹⁴. In some embodiments, X¹⁴ is GlnR. In some embodiments, X¹⁴ is hGlnR. In some embodiments, n1 is 4 as in Lys. In some embodiments, n2 is 2 as in GlnR. In some embodiments, n2 is 3.

In some embodiments, a first amino acid residue is X¹⁰ which is 4PipA. In some embodiments, L^{s1} is —(CH₂)ₙ₁—C(R′)₂—(CH₂)ₙ₃—, wherein each of n1 and n3 is independently n as described herein (e.g., 1-10, 1, 2, 3, 4, 5, 6, 7, 8, 9, or 10), and each R′ is independently as described herein. In some embodiments, one R′ is —H. In some embodiments, n1 is 1. In some embodiments, n3 is 2. In some embodiments, —(CH₂)n3- is connected to —N(R′)— of L^{s2}. In some embodiments, one R′ of —C(R′)₂— of L^{s1} and R′ of —N(R′)— of L^{s2} are taken together with their intervening atoms to form an optionally substituted as described herein. In some embodiments, a formed ring is an optionally substituted 3-10 membered saturated ring. In some embodiments, a formed ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, a formed ring has no additional ring heteroatoms in addition to the nitrogen to which R′ is attached. In some embodiments, L^{s} is -L^{s1}-Cy-C(O)-L^{s3}- wherein each variable is independently as described herein. In some embodiments, -Cy- is optionally substituted

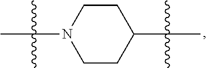 [SMILES: *C1CCN(*)CC1]

wherein the nitrogen atom is bonded to —C(O)—. In some embodiments, each L^{s1} and L^{s3} is independently L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s}-(CH₂)n1-Cy-C(O)—(CH₂)n2- wherein each variable is independently as described herein. In some embodiments, -Cy- is optionally substituted

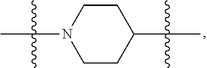 [SMILES: *C1CCN(*)CC1]

wherein the nitrogen atom is bonded to —C(O)—. In some embodiments, n1 is 1. In some embodiments, a second amino acid residue is X¹⁴. In some embodiments, X¹⁴ is GlnR. In some embodiments, n2 is 2.

In some embodiments, a first amino acid residue is X⁷, e.g., GlnR. In some embodiments, n1 is 2. In some embodiments, a second amino acid residue is X¹⁰, e.g., Lys. In some embodiments, n2 is 4. In some embodiments, a first amino acid residue is X⁷, e.g., Lys. In some embodiments, n1 is 4. In some embodiments, a second amino acid residue is X¹⁰, e.g., GlnR. In some embodiments, n2 is 2.

In some embodiments, a first amino acid residue is X¹⁰. In some embodiments, X¹⁰ is GlnR. In some embodiments, X¹⁰ is DGlnR. In some embodiments, n1 is 2. In some embodiments, X¹⁰ is AsnR. In some embodiments, n1 is 1. In some embodiments, —C(O)— of L^{s2} is bonded to L^{s1}. In some embodiments, a first amino acid residue is X¹⁰, e.g., hGlnR. In some embodiments, n1 is 3. In some embodiments, a second amino acid residue is X¹⁴, e.g., iPrLys. In some embodiments, R′ of —N(R′)— of L^{s2} is optionally substituted C₁₋₆ alkyl. In some embodiments, it is isopropyl. In some embodiments, n2 is 4. In some embodiments, a second amino acid residue is X¹⁴, e.g., Lys. In some embodiments, a second amino acid residue is X¹⁴, e.g., Orn. In some embodiments, n2 is 3.

In some embodiments, a second amino acid residue is X¹⁴ which is 4PipA. In some embodiments, L^{s3} is —(CH₂)ₙ₂—C(R′)₂—(CH₂)ₙ₃—, wherein each of n2 and n3 is independently n as described herein (e.g., 1-10, 1, 2, 3, 4, 5, 6, 7, 8, 9, or 10), and each R′ is independently as described herein. In some embodiments, one R′ is —H. In some embodiments, n2 is 1. In some embodiments, n3 is 2. In some embodiments, —(CH₂)n3- is connected to —N(R′)— of L^{s2}. In some embodiments, one R′ of —C(R′)₂— of L^{s3} and R′ of —N(R′)— of L^{s2} are taken together with their intervening atoms to form an optionally substituted as described herein. In some embodiments, a formed ring is an optionally substituted 3-10 membered saturated ring. In some embodiments, a formed ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, a formed ring has no additional ring heteroatoms in addition to the nitrogen to which R′ is attached. In some embodiments, L^{s} is -L″-C(O)-Cy-L^{s3}- wherein each variable is independently as described herein. In some embodiments, -Cy- is optionally substituted

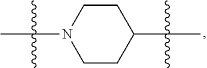 [SMILES: *C1CCN(*)CC1]

wherein the nitrogen atom is bonded to —C(O)—. In some embodiments, each L^{s1} and L^{s3} is independently L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s}-(CH₂)n1-C(O)-Cy-(CH₂)n2- wherein each variable is independently as described herein. In some embodiments, -Cy- is optionally substituted

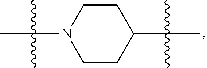 [SMILES: *C1CCN(*)CC1]

wherein the nitrogen atom is bonded to —C(O)—. In some embodiments, n1 is 2. In some embodiments, n2 is 1.

In some embodiments, a second amino acid residue is

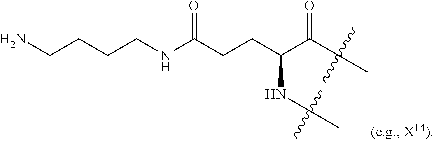 [SMILES: *N[C@@H](CCC(=O)NCCCCN)C(*)=O]

In some embodiments, L^{s3} is —(CH₂)₂—C(O)NH—(CH₂)₄—. In some embodiments, a second amino acid residue is

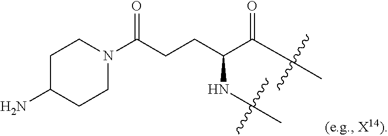 [SMILES: *N[C@@H](CCC(=O)N1CCC(N)CC1)C(*)=O]

In some embodiments, L^{s3} is —(CH₂)₂—C(O)-Cy-. In some embodiments, -Cy- is optionally substituted

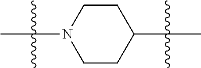 [SMILES: *C1CCN(*)CC1]

wherein the nitrogen is bonded to —C(O)—. In some embodiments, L^{s3} is —(CH₂)₂—C(O)—N(R′)—(CH₂)n-CHR′—, wherein the two R′ are taken together with their intervening atoms to form an optionally substituted ring as described herein. In some embodiments, a formed ring is optionally substituted

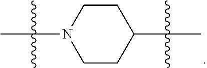 [SMILES: *C1CCN(*)CC1]

In some embodiments, a second amino acid residue is

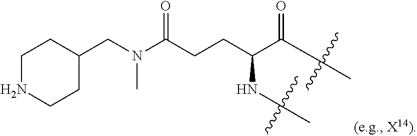 [SMILES: *N[C@@H](CCC(=O)N(C)CC1CCNCC1)C(*)=O]

In some embodiments, L^{s3} is —(CH₂)₂—C(O)—N(R′)—(CH₂)n-Cy-. In some embodiments, R′ is R as described herein. In some embodiments, R is —H. In some embodiments, R optionally substituted C₁₋₆ aliphatic. In some embodiments, R optionally substituted C₁₋₆ alkyl. In some embodiments, R is methyl. In some embodiments, n is 1. In some embodiments, -Cy- is optionally substituted

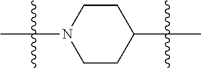 [SMILES: *C1CCN(*)CC1]

wherein the nitrogen is bonded to L^{s2} which is or comprises —C(O)—. In some embodiments, L^{s3} is —(CH₂)₂—C(O)—N(R′)—CH₂—CHR′—(CH₂)n-. In some embodiments, n is 2. In some embodiments, —(CH₂)n- is bonded to —N(R′)— of L^{s2} which is —C(O)—N(R′)—. In some embodiments, R′ of —CHR′— of L^{s3} is taken together with R′ of —N(R′)— of L^{s2} and their intervening atoms to form an optionally substituted ring as described herein. In some embodiments, a formed ring is optionally substituted

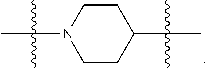 [SMILES: *C1CCN(*)CC1]

In some embodiments, a second amino acid residue is

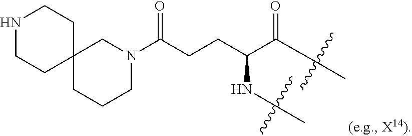 [SMILES: *N[C@@H](CCC(=O)N1CCCC2(CCNCC2)C1)C(*)=O]

In some embodiments, a second amino acid residue is

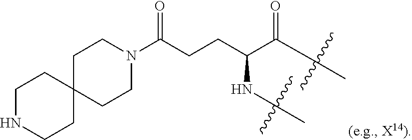 [SMILES: *N[C@@H](CCC(=O)N1CCC2(CCNCC2)CC1)C(*)=O]

In some embodiments, L^{s3}-(CH₂)₂—C(O)—N(R′)—(CH₂)ₙ₁—C(R′)₂—(CH₂)ₙ₂—. In some embodiments, each of n1 and n2 is independently 1-10. In some embodiments, n1 is 1. In some embodiments, n1 is 2. In some embodiments, n2 is 2. In some embodiments, R′ of —N(R′)— and one R′ of —C(R′)₂— are taken together with their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, L^{s2} is —C(O)N(R′)—. In some embodiments, —N(R′)— is bonded to —(CH₂)ₙ₂—. In some embodiments, one R′ of —C(R′)₂— of L^{s3} is taken together with R′ of —N(R′)— of L^{s2} and their intervening atoms to form an optionally substituted ring as described herein. In some embodiments, a formed ring is an optionally substituted 6-membered monocyclic saturated ring having no heteroatoms in addition to the nitrogen atom of —N(R′)—.

In some embodiments, a first amino acid residue is

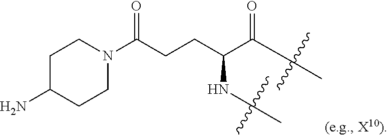 [SMILES: *N[C@@H](CCC(=O)N1CCC(N)CC1)C(*)=O]

In some embodiments, L^{s1} is —(CH₂)₂—C(O)—N(R′)—(CH₂)n-CHR′—, wherein the two R′ are taken together with their intervening atoms to form an optionally substituted ring as described herein. In some embodiments, a formed ring is optionally substituted

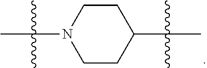 [SMILES: *C1CCN(*)CC1]

In some embodiments, a second amino acid residue is GlnR (e.g., X⁴). In some embodiments, L^{s3} is —(CH₂)₂—.

In some embodiments, a first amino acid residue is

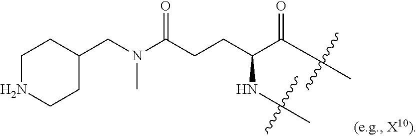 [SMILES: *N[C@@H](CCC(=O)N(C)CC1CCNCC1)C(*)=O]

In some embodiments, L^{s1} is —(CH₂)₂—C(O)—N(R′)—(CH₂)n-Cy-. In some embodiments, R′ is R as described herein. In some embodiments, R is —H. In some embodiments, R optionally substituted C₁₋₆ aliphatic. In some embodiments, R optionally substituted C₁₋₆ alkyl. In some embodiments, R is methyl. In some embodiments, n is 1. In some embodiments, -Cy- is optionally substituted

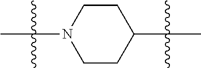 [SMILES: *C1CCN(*)CC1]

wherein the nitrogen is bonded to L^{s2} which is or comprises —C(O)—. In some embodiments, L″ is —(CH₂)₂—C(O)—N(R′)—CH₂—CHR′—(CH₂)n-. In some embodiments, n is 2. In some embodiments, —(CH₂)n- is bonded to —N(R′)— of L^{s2} which is —C(O)—N(R′)—. In some embodiments, R′ of —CHR′— of L^{s} is taken together with R′ of —N(R′)— of L^{s2} and their intervening atoms to form an optionally substituted ring as described herein. In some embodiments, a formed ring is optionally substituted

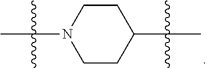 [SMILES: *C1CCN(*)CC1]

In some embodiments, a first amino acid residue is

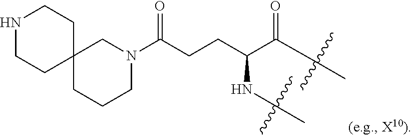 [SMILES: *N[C@@H](CCC(=O)N1CCCC2(CCNCC2)C1)C(*)=O]

In some embodiments, a first amino acid residue is

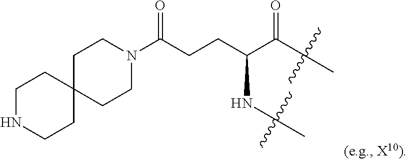 [SMILES: *N[C@@H](CCC(=O)N1CCC2(CCNCC2)CC1)C(*)=O]

In some embodiments, L^{s1}-(CH₂)₂—C(O)—N(R′)—(CH₂)ₙ₁—C(R′)₂—(CH₂)ₙ₂—. In some embodiments, each of n1 and n2 is independently 1-10. In some embodiments, n1 is 1. In some embodiments, n1 is 2. In some embodiments, n2 is 2. In some embodiments, R′ of —N(R′)— and one R′ of —C(R′)₂— are taken together with their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, L^{s2} is —C(O)N(R′)—. In some embodiments, —N(R′)— is bonded to —(CH₂)ₙ₂—. In some embodiments, one R′ of —C(R′)₂— of L^{s1} is taken together with R′ of —N(R′)— of L^{s2} and their intervening atoms to form an optionally substituted ring as described herein. In some embodiments, a formed ring is an optionally substituted 6-membered monocyclic saturated ring having no heteroatoms in addition to the nitrogen atom of —N(R′)—. In some embodiments, a second amino acid residue is GlnR (e.g., X¹⁴).

In some embodiments, a first residue is

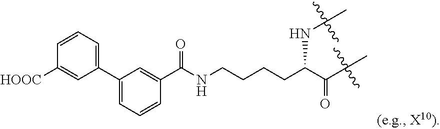 [SMILES: *N[C@@H](CCCCNC(=O)c1cccc(-c2cccc(C(=O)O)c2)c1)C(*)=O]

In some embodiments, a first residue is

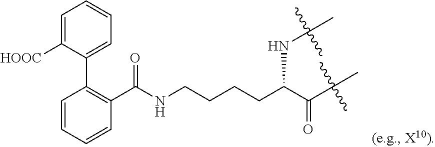 [SMILES: *N[C@@H](CCCCNC(=O)c1ccccc1-c1ccccc1C(=O)O)C(*)=O]

In some embodiments, a first residue is

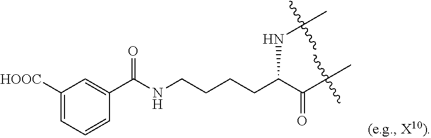 [SMILES: *N[C@@H](CCCCNC(=O)c1cccc(C(=O)O)c1)C(*)=O]

In some embodiments, L^{s1} is —(CH₂)n-N(R′)—C(O)-Cy-Cy-, wherein each variable is independently as described herein. In some embodiments, L^{s1} is —(CH₂)n-N(R′)—C(O)-Cy-, wherein each variable is independently as described herein. In some embodiments, a first residue is

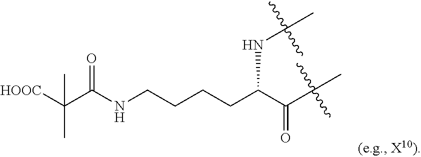 [SMILES: *N[C@@H](CCCCNC(=O)C(C)(C)C(=O)O)C(*)=O]

In some embodiments, L^{s1} is —(CH₂)n-N(R′)—C(O)—CH₂—, wherein R is as described herein, and the —CH₂— bonded to C(O)— is optionally substituted. In some embodiments, L^{s1} is —(CH₂)n-N(R′)—C(O)—C(R′)₂—, wherein each R is independently as described herein. In some embodiments, L^{s1} is —(CH₂)n-N(R′)—C(O)—C(CH₃)₂—, wherein R is as described herein. In some embodiments, a first residue is

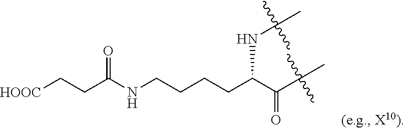 [SMILES: *N[C@@H](CCCCNC(=O)CCC(=O)O)C(*)=O]

In some embodiments, L^{s1} is —(CH₂)ₙ₁—N(R′)—C(O)—(CH₂)ₙ₂—, wherein each variable is independently as described herein. In some embodiments, each of n1 and n2 is independently n as described herein. In some embodiments, L^{s1} is —(CH₂)₄—N(R′)—C(O)—(CH₂)₂—, wherein each R is independently as described herein. In some embodiments, n is 1-10. In some embodiments, n is 1. In some embodiments, n is 2. In some embodiments, n is 3. In some embodiments, n is 4. In some embodiments, R′ is R as described herein. In some embodiments, R is —H. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s2} is or comprises —C(O)—N(R′)— as described herein. In some embodiments, R′ is R as described herein. In some embodiments, R is —H. In some embodiments, L^{s2} is —C(O)NH—. In some embodiments, —C(O)— is bonded to -Cy- of L^{s1}. In some embodiments, a second residue is X⁴, e.g., Lys. In some embodiments, L^{s3} is as described herein, e.g., optionally substituted —(CH₂)n-. In some embodiments, L^{s3} is —(CH₂)n-. In some embodiments, n is 1. In some embodiments, n is 2. In some embodiments, n is 3. In some embodiments, n is 4 (e.g., as in Lys).

In some embodiments, R^{SP1} of a one amino acid residue in a pair is a first reaction group of a cycloaddition reaction. In some embodiments, such an amino acid residue can be stapled with another amino acid residue comprising a second reactive group of a cycloaddition reaction through a cycloaddition reaction. In some embodiments, in the other amino acid residue of a pair R^{SP1} is a second reactive group of a cycloaddition reaction. In some embodiments, a cycloaddition reaction is [3+2]. In some embodiments, a cycloaddition reaction is a click chemistry reaction. In some embodiments, a cycloaddition reaction is [4+2]. In some embodiments, one of the first and the second reactive groups is or comprises —N₃, and the other is or comprises an alkyne (e.g., a terminal alkyne or activated/strained alkyne).

In some embodiments, R^{SP1} of a first amino acid residue is —N₃. In some embodiments, L^{a} of a first amino acid residue is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, R^{SP1} of a second amino acid residue is or comprises —C≡C—. In some embodiments, R^{SP1} of a second amino acid residue is —≡—H. In some embodiments, R^{SP1} of a second amino acid residue comprises a strained alkyne, e.g., in a ring. In some embodiments, L^{a} of a first amino acid residue is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋ₙ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, L^{s} is -L^{s1}-L^{s2}-L^{s3}-, wherein L^{s2} is or comprises -Cy-. In some embodiments, L^{s2} is -Cy-. In some embodiments, -Cy- is formed by a cycloaddition reaction. In some embodiments, -Cy- is optionally substituted

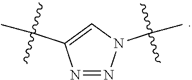 [SMILES: *c1cn(*)nn1]

In some embodiments, -Cy- is

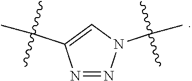 [SMILES: *c1cn(*)nn1]

In some embodiments, -Cy- is optionally substituted

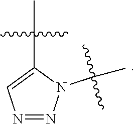 [SMILES: *c1cnnn1*]

In some embodiments, -Cy- is

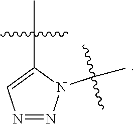 [SMILES: *c1cnnn1*]

In some embodiments, L^{s1} is L^{a} of a first amino acid residue, and L^{s3} is L^{a} of a second amino acid residue. In some embodiments, L^{s1} is L^{a} of a second amino acid residue, and L^{s3} is L^{a} of a first amino acid residue. In some embodiments, each of L^{s1} and L^{s3} is independently L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s1} is optionally substituted —(CH₂)ₙ—, wherein n is 1-10. In some embodiments, L^{s1} is —(CH₂)ₙ—, wherein n is 1-10. In some embodiments, n is 1. In some embodiments, n is 2. In some embodiments, n is 3. In some embodiments, n is 4. In some embodiments, L^{s3} is optionally substituted —(CH₂)ₙ—, wherein n is 1-10. In some embodiments, L^{s3} is —(CH₂)ₙ—, wherein n is 1-10. In some embodiments, n is 1. In some embodiments, n is 2. In some embodiments, n is 3. In some embodiments, n is 4.

In some embodiments, a first amino acid residue is X¹⁰. In some embodiments, R^{SP1} of X¹⁰ is —N₃. In some embodiments, L^{a} of X¹⁰ is optionally substituted —(CH₂)n- wherein n is 1-10. In some embodiments, L^{a} of X¹⁰ is —(CH₂)₄—. In some embodiments, L^{a} of X¹⁰ is —(CH₂)₃—. In some embodiments, L^{a} of X¹¹ is —(CH₂)₂—. In some embodiments, L^{a} of X¹¹ is —CH₂—. In some embodiments, a second amino acid residue is X¹⁴. In some embodiments, R^{SP1} of X¹⁴ is or comprises an alkyne, e.g., a strained/activated alkyne. In some embodiments, R^{SP1} of X¹⁴ is —C≡CH. In some embodiments, L^{a} of X¹⁴ is optionally substituted —(CH₂)n- wherein n is 1-10. In some embodiments, L^{a} of X¹⁴ is —(CH₂)₄—. In some embodiments, L^{a} of X¹⁴ is —(CH₂)₃—. In some embodiments, L^{a} of X¹⁴ is —(CH₂)₂—. In some embodiments, L^{a} of X¹⁴ is —CH₂—. In some embodiments, a methylene unit is replaced with —O—. In some embodiments, L^{a} of X¹⁴ is —CH₂—O—CH₂—. In some embodiments, L^{s3} is L^{a} of X¹⁴. In some embodiments, L^{s3} is bonded to a carbon atom of L^{s2}.

In some embodiments, a first amino acid residue is X¹⁰. In some embodiments, R^{SP1} of X¹⁰ is or comprises an alkyne, e.g., a strained/activated alkyne. In some embodiments, R^{SP1} of X¹⁰ is —C≡CH. In some embodiments, L^{a} of X¹⁰ is optionally substituted —(CH₂)n- wherein n is 1-10. In some embodiments, L^{a} of X¹⁰ is —(CH₂)₄—. In some embodiments, L^{a} of X¹⁰ is —(CH₂)₃—. In some embodiments, L^{a} of X¹⁰ is —(CH₂)₂—. In some embodiments, L^{a} of X¹⁰ is —CH₂—. In some embodiments, a methylene unit is replaced with —O—. In some embodiments, L^{a} of X¹⁰ is —CH₂—O—CH₂—. In some embodiments, L^{s1} is L^{a} of X¹⁰. In some embodiments, L^{s1} is bonded to a carbon atom of L^{s2}.In some embodiments, a second amino acid residue is X¹⁴. In some embodiments, R^{SP1} of X¹⁴ is —N₃. In some embodiments, L^{a} of X¹⁴ is optionally substituted —(CH₂)n- wherein n is 1-10. In some embodiments, L^{a} of X¹⁴ is —(CH₂)₄—. In some embodiments, L^{a} of X¹⁴ is —(CH₂)₃—. In some embodiments, L^{a} of X¹⁴ is —(CH₂)₂—. In some embodiments, L^{a} of X¹⁴ is —CH₂—.

In some embodiments, R^{SP1} is a nucleophile. In some embodiments, R^{SP1} is —SH, e.g., as in Cys. In some embodiments, L^{s2} is L″ as described herein. In some embodiments, L^{s2} is —S—CH₂-L″-CH₂—S— wherein L^{s1} is as described herein. In some embodiments, a staple has the structure of -L^{s1}-S—CH₂-L″-CH₂—S-L^{s3}, wherein each variable is independently as described herein, and each —CH₂— is optionally substituted. In some embodiments, L^{s2} is —S—C(R′)₂-L″-C(R′)₂—S—, wherein each variable is independently as described herein. In some embodiments, a staple has the structure of -L^{s1}-S—C(R′)₂-L″-C(R′)₂—S-L^{s3}-, wherein each variable is independently as described herein. In some embodiments, each R′ is independently R as described herein. In some embodiments, each R′ is —H. In some embodiments, L^{s2} is —S-Cy-S— wherein -Cy- is as described herein. In some embodiments, a staple has the structure of -L^{s1}-S-Cy-S-L^{s3}-, wherein each variable is independently as described herein. In some embodiments, L^{s2} is —S-Cy-Cy-S— wherein -Cy- is as described herein. In some embodiments, a staple has the structure of -L^{s1}-S-Cy-Cy-S-L^{s3}-, wherein each variable is independently as described herein. In some embodiments, L^{s1} is L^{a} of a first amino acid residue. In some embodiments, L^{s3} is L^{a} of a second amino acid residue. In some embodiments, each of L^{s1} and L^{s3} is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, each of a pair of amino acid residues is Cys. In some embodiments, L^{s1} is —CH₂—. In some embodiments, L^{s3} is —CH₂—. In some embodiments, L^{s1} is -Cy- as described herein. In some embodiments, -Cy- is optionally substituted phenylene. In some embodiments, -Cy- is phenylene. In some embodiments, -Cy- is optionally substituted 1,2-phenylene. In some embodiments, -Cy- is 1,2-phenylene. In some embodiments, -Cy- is optionally substituted 1,3-phenylene. In some embodiments, -Cy- is 1,3-phenylene. In some embodiments, -Cy- is optionally substituted 1,4-phenylene. In some embodiments, -Cy- is tetrafluoro-1,4-phenylene. In some embodiments, -Cy- is 1,4-phenylene. In some embodiments, -Cy- is optionally substituted naphthylene. In some embodiments, -Cy- is optionally substituted

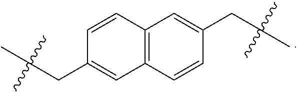 [SMILES: *Cc1ccc2cc(C*)ccc2c1]

In some embodiments, L^{s1} is -Cy-Cy-, wherein each -Cy- is independently as described herein. In some embodiments, each -Cy- is independently optionally substituted phenylene. In some embodiments, each -Cy- is independently phenylene. In some embodiments, each -Cy- is independently optionally substituted 1,2-phenylene. In some embodiments, each -Cy- is independently 1,2-phenylene. In some embodiments, each -Cy- is independently optionally substituted 1,3-phenylene. In some embodiments, each -Cy- is independently 1,3-phenylene. In some embodiments, each -Cy- is independently optionally substituted 1,4-phenylene. In some embodiments, each -Cy- is independently 1,4-phenylene. In some embodiments, each -Cy- is independently tetrafluoro-1,4-phenylene.

As appreciated by those skilled in the art, such staples may be formed by linking Cys residues with a linking reagent having the structure of R^{x}-L^{s2}-R^{x}, wherein each variable is independently as described herein. In some embodiments, each R^{x} is —Br.

In some embodiments, R^{SP1} of two amino acid residues of a pair of amino acid residues suitable for stapling can each independently react with a linking reagent to form a staple. In some embodiments, a suitable linking reagent comprises two reactive groups, each can independently react with R^{SP1} of each amino acid residue. In some embodiments, a linking reagent has the structure of H-L″-H or a salt thereof, wherein the reagent comprises two amino groups, and L″ is a covalent bond, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, such a linking agent can react with two amino acid residues each independently having a R^{SP1} group that is —COOH or an activated form thereof.

Suitable embodiments for L″ including those described for L herein that fall within the scope of L″. For example, in some embodiments, L″ is L wherein L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, a linking reagent is a diamine or a salt thereof. In some embodiments, a reagent has the structure of NHR-L″-NHR or a salt thereof, wherein each variable is independently as described herein. In some embodiments, each R is independently —H or optionally substituted C₁₋₆ aliphatic. In some embodiments, each R is independently —H or C₁₋₆ aliphatic. In some embodiments, each R is independently —H or optionally substituted C₁₋₆ alkyl. In some embodiments, each R is independently —H or C₁₋₆ alkyl. In some embodiments, a reagent has the structure of NH₂-L″-NH₂ or a salt thereof. In some embodiments, L^{s1} is optionally substituted —(CH₂)n- wherein n is 1-10. In some embodiments, L″ is —(CH₂)₄—.

In some embodiments, a staple, L^{s}, is -L^{s1}-L^{s2}-L^{s3}-, wherein L^{s1} is L^{a} of a first amino acid residue of a stapled pair, L^{s3} is L^{a} of a second amino acid residue of a stapled pair, and L^{s2} is —C(O)—N(R′)-L″-N(R′)—C(O)—, wherein each variable is independently as described herein. In some embodiments, L″ is optionally substituted —(CH₂)n- wherein n is 1-10. In some embodiments, L″ is —(CH₂)₄—. In some embodiments, each of L^{s} and L^{s3} is independently optionally substituted —(CH₂)n- wherein n is 1-10. In some embodiments, n is 2. In some embodiments, a first amino acid residue is Gln (e.g., X¹⁰). In some embodiments, a second amino acid residue is GlnR (e.g., X¹⁴). In some embodiments, two GlnR can form such a staple through [diaminobutane].

In some embodiments, a linking reagent has the structure of H-Cy-L″-NHR or a salt thereof, wherein -Cy- comprises a second amino group. In some embodiments, R is —H or optionally substituted C₁₋₆ aliphatic. In some embodiments, R is —H or C₁₋₆ aliphatic. In some embodiments, R is —H or optionally substituted C₁₋₆ alkyl. In some embodiments, R is —H or C₁₋₆ alkyl. In some embodiments, R is methyl. In some embodiments, a linking reagent has the structure of H-Cy-L″-NH₂ or a salt thereof, wherein -Cy-comprises a second amino group. In some embodiments, -Cy- is optionally substituted

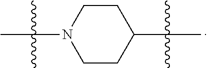 [SMILES: *C1CCN(*)CC1]

In some embodiments, -Cy- is

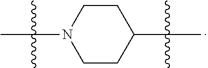 [SMILES: *C1CCN(*)CC1]

In some embodiments, L^{s1} is a covalent bond. In some embodiments, L^{s1} is optionally substituted —(CH₂)n- wherein n is 1-10. In some embodiments, L″ is —(CH₂)—. In some embodiments, a linking reagent is

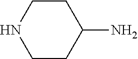

or a salt thereof. In some embodiments, a linking reagent is

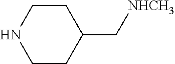

or a salt thereof.
as described herein. In some embodiments, R′ is —H. In some embodiments, -Cy- is

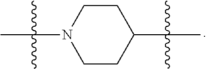 [SMILES: *C1CCN(*)CC1]

In some embodiments, each of L^{s1} and L^{s3} is independently optionally substituted —(CH₂)n- wherein n is 1-10. In some embodiments, n is 2. In some embodiments, -Cy- is closer to a N-terminus than —N(R′)—. In some embodiments, -Cy- is closer to a C-terminus than —N(R′)—. In some embodiments, a first amino acid residue is Gln (e.g., X¹⁰). In some embodiments, a second amino acid residue is GlnR (e.g., X¹⁴). In some embodiments, two GlnR can form such a staple through [4aminopiperidine].

In some embodiments, L^{s2} is —C(O)-Cy-(CH₂)n-N(R′)—C(O)—, wherein each variable is independently as described herein. In some embodiments, R′ is —H. In some embodiments, R′ is R as described herein, e.g., optionally substituted C₁₋₆ aliphatic, C₁₋₆ alkyl, etc. In some embodiments, R is methyl. In some embodiments, n is 1. In some embodiments, -Cy- is

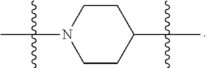 [SMILES: *C1CCN(*)CC1]

In some embodiments, -Cy- is closer to a N-terminus than —N(R′)—. In some embodiments, -Cy- is closer to a C-terminus than —N(R′)—. In some embodiments, each of L^{s1} and L^{s3} is independently optionally substituted —(CH₂)n- wherein n is 1-10. In some embodiments, n is 2. In some embodiments, a first amino acid residue is Gln (e.g., X¹⁰). In some embodiments, a second amino acid residue is GlnR (e.g., X¹⁴). In some embodiments, two GlnR can form such a staple through [4mampiperidine].

In some embodiments, a methylene unit is replaced with -Cy-. In some embodiments, a linking reagent has the structure of H-Cy-H, wherein Cy comprises two secondary amino groups. In some embodiments, -Cy- is optionally substituted 8-20 membered bicyclic ring. In some embodiments, H-Cy-H comprises two —NH—. In some embodiments, -Cy- is optionally substituted

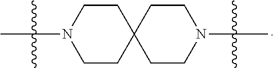 [SMILES: *N1CCC2(CC1)CCN(*)CC2]

In some embodiments, -Cy- is optionally substituted

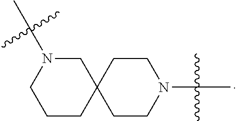 [SMILES: *N1CCC2(CCCN(*)C2)CC1]

In some embodiments, the meta connection site (relative to the spiro carbon atom) is closer to a N-terminus than the para connection site (relative to the spiro carbon atom). In some embodiments, the meta connection site (relative to the spiro carbon atom) is closer to a C-terminus than the para connection site (relative to the spiro carbon atom).

In some embodiments, L^{s2} is —C(O)-Cy-C(O)— wherein -Cy- is as described herein. In some embodiments, each of L^{s1} and L^{s3} is independently optionally substituted —(CH₂)n- wherein n is 1-10. In some embodiments, n is 2. In some embodiments, a first amino acid residue is Gln (e.g., X¹⁰). In some embodiments, a second amino acid residue is GlnR (e.g., X⁴). In some embodiments, two GlnR can form such a staple through [29N2spiroundecane]. In some embodiments, two GlnR can form such a staple through [39N2spiroundecane].

In some embodiments, a pair of amino acid residue suitable for stapling both independently has the structure of —N(R^{a1})-L^{a}-C(-L^{a}-R^{SP1})(R³)-L^{a2}-C(O)— or —N(R^{a1})—C(-L^{a}-R^{SP1})(R^{a3})—C(O)—, wherein each variable is independently as described herein, and R^{SP1} is an amino group. In some embodiments, R^{SP1} is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is C₁₋₆ aliphatic. In some embodiments, R is C₁₋₆ alkyl. In some embodiments, R^{SP1} is —NH₂. In some embodiments, such two amino acid residue may be linked by a di-acid linking reagent.

In some embodiments, a linking reagent has the structure of HOOC-L″-COOH, or a salt thereof, or an activated form thereof, wherein L^{s1} is as described herein. In some embodiments, L^{s1} is -Cy-Cy-. In some embodiments, L^{s1} is -Cy-. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s1} is optionally substituted

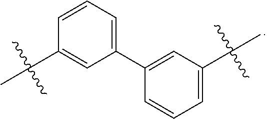 [SMILES: *c1cccc(-c2cccc(*)c2)c1]

In some embodiments, a linking agent is

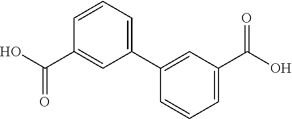 [SMILES: O=C(O)c1cccc(-c2cccc(C(=O)O)c2)c1]

or a salt or an activated form thereof. In some embodiments, L^{s1} is optionally substituted

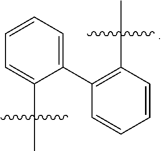 [SMILES: *c1ccccc1-c1ccccc1*]

In some embodiments, a linking agent is

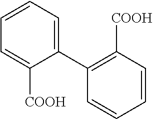 [SMILES: O=C(O)c1ccccc1-c1ccccc1C(=O)O]

or a salt or an activated form thereof. In some embodiments, L″ is 1,3-phenylene. In some embodiments, a linking agent is

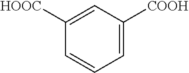 [SMILES: O=C(O)c1cccc(C(=O)O)c1]

or a salt or an activated form thereof. In some embodiments, L″ is optionally substituted —(CH₂)n-, wherein n is 1-10. In some embodiments, L″ is optionally substituted —CH₂—. In some embodiments, L″ is —C(R′)₂—. In some embodiments, L^{s1} is —C(CH₃)₂—. In some embodiments, a linking agent is (CH₃)₂C(COOH)₂ or a salt or an activated form thereof. In some embodiments, L″ is —CH₂CH₂—. In some embodiments, a linking agent is HOOCCH₂CH₂COOH or a salt or an activated form thereof.

In some embodiments, a staple is L^{s}, wherein L^{s2} is —N(R′)-L″-N(R′)—, and each of L^{s1} and L^{s3} is independently as described herein. In some embodiments, L″ is -Cy-Cy-, wherein each -Cy- is independently as described herein. In some embodiments, L″ is -Cy- as described herein. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, each -Cy- is independently optionally substituted 1,2-phenylene. In some embodiments, each -Cy- is independently optionally substituted 1,3-phenylene. In some embodiments, L^{s1} is optionally substituted —(CH₂)n-, wherein n is 1-10. In some embodiments, L^{s1} is optionally substituted —CH₂—. In some embodiments, L^{s1} is —C(R′)₂—. In some embodiments, L^{s1} is —C(CH₃)₂—. In some embodiments, L″ is —CH₂CH₂—. In some embodiments, each of L^{s1} and L^{s3} is independently optionally substituted —(CH₂)n- wherein n is 1-10. In some embodiments, n is 2. In some embodiments, n is 4. In some embodiments, a first amino acid residue is Lys (e.g., X¹⁰). In some embodiments, a second amino acid residue is Lys (e.g., X¹⁴). In some embodiments, two Lys can form such a staple through [Biphen33COOH]. In some embodiments, two Lys can form such a staple through [diphenate]. In some embodiments, two Lys can form such a staple through [isophthalate]. In some embodiments, two Lys can form such a staple through [Me2Mal]. In some embodiments, two Lys can form such a staple through [succinate].

In some embodiments, X¹⁰ is stapled. In some embodiments, X¹⁰ is stapled with X¹⁴. In some embodiments, X¹⁰ is stapled with X⁷.

In some embodiments, X¹⁰ is Lys, Phe, TriAzLys, GlnR, Leu, PyrS2, Aib, Ala, sAla, AsnR, hGlnR, dOm, PyrS1, dLys, dDab, [mPyr]Cys, PyrS3, iPrLys, [mXyl]Cys, TriAzOm, 1MeK, [C3]Cys, [IsoE]Cys, DGlnR, Orn, [mPyr]hCys, [Red] Cys, [C3]hCys, 4PipA, sCH2S, [8FBB]Cys, [pXyl]Cys, [pXyl]hCys, [33Oxe]Cys, [Red]hCys, [IsoE]hCys, [13Ac]hCys, [m5Meb]Cys, [m5Meb]hCys, GlnS3APyr, AsnMeEDA, AsnR3APyr, [m5Pyr]Cys, [m50Meb]Cys, [4FB]Cys, [oXyl]Cys, NMeOm, [2-6-naph]Cys, [3-3-biph]Cys, [mXyl]hCys, [3-3-biPh]hCys, [2-6-naph]hCys, [33Oxe]hCys, [13Ac]Cys, GlnR3APyr, AsnS3APyr, [IsoE]hCysOx, or [m5Pyr]hCys. In some embodiments, X¹⁰ is Lys. In some embodiments, X¹⁰ is Phe. In some embodiments, X¹⁰ is TriAxLys. In some embodiments, X¹⁰ is GlnR. In some embodiments, X¹⁰ is Leu. In some embodiments, X¹⁰ is PryS2. In some embodiments, X¹⁰ is Aib. In some embodiments, X¹⁰ is Ala. In some embodiments, X¹⁰ is Val.

In some embodiments, X¹⁰ is or comprises a residue of an amino acid or a moiety selected from Table A-I, Table A-II, Table A-III and Table A-IV.

Various types of amino acid residues can be used for X¹¹, e.g., a residue of an amino acid of formula A-I, A-II, A-III, A-IV, A-V, A-VI, etc. or a salt thereof in accordance with the present disclosure. In some embodiments, X¹¹ is —N(R^{a1})-L^{a1}-C(R^{a2})(R^{a3})-L^{a2}-C(O)—, wherein each variable is independently as described herein. In some embodiments, X¹¹ is —N(R^{a1})—C(R^{a2})(R^{a1})C(O)—, wherein each variable is independently as described herein. In some embodiments, X¹¹ is —N(R^{a1})—C(R^{a2})H—C(O)—, wherein each variable is independently as described herein. In some embodiments, R^{a1} is —H. In some embodiments, R^{a3} is —H.

In some embodiments, X¹¹ is a residue of an amino acid suitable for stapling as described herein. In some embodiments, an amino acid residue suitable for stapling is —N(R^{a1})-L^{a1}-C(-L^{a}-R^{SP1})(R^{a3})-L^{a2}-C(O)— wherein each variable is independently as described herein. In some embodiments, it is —N(R^{a1})—C(-L^{a}-R^{SP1})(R^{a3})—C(O)— wherein each variable is independently as described herein. In some embodiments, in a pair of amino acid residues suitable for stapling, each amino acid residue is independently —N(R^{a1})-L^{a1}-C(-L^{a}-R^{SP1})(R^{a3})-L^{a2}-C(O)— or —N(R^{a1})—C(-L^{a}-R^{SP1})(R^{a3})—C(O)—, wherein each variable is independently as described herein. In some embodiments, R^{a1} is —H. In some embodiments, R^{a3} is —H. In some embodiments, both R^{a1} and R^{a3} are —H. In some embodiments, R^{SP1} comprises optionally substituted —CH═CH—. In some embodiments, R^{SP1} is or comprises optionally substituted —CH═CH₂. In some embodiments, R^{SP1} is —CH═CH₂.

In some embodiments, X¹¹ is a residue of an amino acid, e.g., having the structure of formula A-I, A-II, A-III, A-IV, A-V, A-VI, etc., whose side chain comprise a functional group suitable for stapling, e.g., a double bond. In some embodiments, X¹¹ is a residue of an amino acid that comprises one and no more than one functional groups for stapling. In some embodiments, X¹¹ is a residue of an amino acid that comprises one and no more than one double bond for stapling. As in certain embodiments of X¹, in some embodiments, X¹¹ comprises a ring structure, and its amino group is part of a ring. In some embodiments, X¹ is an amino acid as described herein (e.g., of formula A-I, A-II, A-III, etc.), wherein R^{a1} and R^{a3} are taken together to form an optionally substituted ring, e.g., an optionally substituted 3-10 membered ring. In some embodiments, R^{a1} and R^{a3} are taken together with their intervening atoms to form an optionally substituted 3-10 membered saturated or partially saturated ring having, in addition to the intervening atoms, 0-5 heteroatoms.

In some embodiments, R^{a2} and R^{a3} are taken together to form an optionally substituted ring, e.g., an optionally substituted 3-10 membered ring. In some embodiments, R^{a2} and R^{a3} are taken together with their intervening atoms to form an optionally substituted 3-10 membered saturated or partially saturated ring having, in addition to the intervening atoms, 0-5 heteroatoms.

As described herein, in some embodiments, a formed ring, e.g., by R^{a1} and R^{a3} taken together with their intervening atoms, by R^{a2} and R^{a3} taken together with their intervening atoms, or by any other two suitable R taken together with their intervening atoms, either in X¹¹ or another moiety, is saturated. In some embodiments, a formed ring is monocyclic. In some embodiments, a formed ring has no heteroatoms in addition to the intervening atoms. In some embodiments, a formed ring has at least one heteroatom in addition to the intervening atoms. In some embodiments, a formed ring has at least one nitrogen in addition to the intervening atoms. In some embodiments, L^{a1} and L^{a2} are covalent bond. In some embodiments, a formed ring is unsubstituted. In some embodiments, a formed ring is substituted. In some embodiments, a substituent comprises a double bond which is suitable for metathesis with another double bond to form a staple. In some embodiments, a substituent has the structure of —C(O)—O—(CH₂)n-CH═CH₂, wherein n is 1, 2, 3, 4, 5, 6, 7, 8, 9, or 10. In some embodiments, a substituent bonds to a nitrogen ring atom (e.g., see PyrS2). In some embodiments, X¹¹ is a residue of PyrS2.

In some embodiments, L^{a} is —(CH₂)ₙ₁—N(R′)—C(O)—(CH₂)ₙ₂—, wherein each variable is independently as described herein, and each —CH₂— is optionally substituted. In some embodiments, L^{a} is —(CH₂)ₙ₁—N(R′)—C(O)—(CH₂)ₙ₂—, wherein each variable is independently as described herein. In some embodiments, —(CH₂)ₙ₁— is bonded to X¹¹. In some embodiments, n1 is 1, 2, 3, 4, 5, 6, 7, 8, 9, or 10. In some embodiments, n1 is 1. In some embodiments, n1 is 2. In some embodiments, n1 is 3. In some embodiments, n2 is 1, 2, 3, 4, 5, 6, 7, 8, 9, or 10. In some embodiments, n2 is 1. In some embodiments, n2 is 2. In some embodiments, n2 is 3. In some embodiments, n2 is 4. In some embodiments, n2 is 5. In some embodiments, R′ of —N(R′)— of L^{a} and R^{a3} are taken together with their intervening atoms to form an optionally substituted ring. In some embodiments, a formed ring is optionally substituted 3-10 membered monocyclic, saturated or partially unsaturated ring having, in addition to the nitrogen atom to which R′ is attached, 0-3 heteroatoms. In some embodiments, a formed ring is saturated. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring has no ring heteroatoms other than the nitrogen atom to which R′ is attached. In some embodiments, X¹¹ is a residue of PyrS2.

In some embodiments, X¹¹ is stapled. In some embodiments, X¹¹ is stapled with X⁴. In some embodiments, X¹¹ is PyrS2 and stapled. In some embodiments, X¹¹ is Lys and stapled.

In some embodiments, X¹¹ is a residue of PyrS2 or Lys.

In some embodiments, X¹¹ is a residue of PyrS2 and stapled.

In some embodiments, a staple, e.g., L^{s}, has the structure of -L^{s1}-L^{s2}-L^{s3}, wherein each variable is independently as described herein. In some embodiments, L^{s1} or L^{s3} is L^{a} of X¹¹ as described herein. In some embodiments, L^{s3} is L^{a} of X¹¹ as described herein. In some embodiments, L^{s1} is L^{a} of another amino acid residue, e.g., X⁴. In some embodiments, L^{s1} is L as described herein. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—. In some embodiments, L^{s3} is L as described herein. In some embodiments, L^{s3} is —(CH₂)ₙ₁—N(R′)—C(O)—(CH₂)ₙ₂—, wherein each variable is independently as described herein, and each —CH₂— is optionally substituted. In some embodiments, L^{s3} is —(CH₂)ₙ₁—N(R′)—C(O)—(CH₂)n2-, wherein each variable is independently as described herein. In some embodiments, —(CH₂)ₙ₁— is bonded to X¹¹. In some embodiments, n1 is 1, 2, 3, 4, 5, 6, 7, 8, 9, or 10. In some embodiments, n1 is 1. In some embodiments, n1 is 2. In some embodiments, n1 is 3. In some embodiments, n2 is 1, 2, 3, 4, 5, 6, 7, 8, 9, or 10. In some embodiments, n2 is 1. In some embodiments, n2 is 2. In some embodiments, n2 is 3. In some embodiments, n2 is 4. In some embodiments, n2 is 5. In some embodiments, R′ of —N(R′)— of L^{a} and R^{a3} are taken together with their intervening atoms to form an optionally substituted ring. In some embodiments, a formed ring is optionally substituted 3-10 membered monocyclic, saturated or partially unsaturated ring having, in addition to the nitrogen atom to which R′ is attached, 0-3 heteroatoms. In some embodiments, a formed ring is saturated. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring has no ring heteroatoms other than the nitrogen atom to which R′ is attached.

In some embodiments, L^{s2} is optionally substituted —CH═CH—. In some embodiments, L^{s2} is —CH═CH—. In some embodiments, L^{s2} is optionally substituted —CH₂—CH₂—. In some embodiments, L^{s2} is —CH₂—CH₂—.

In some embodiments, X¹¹ is PyrS2, Lys, 3Thi, Ala, Phe, SPip3, PyrSadNip3Butene, SPip2, Az3, DapAc7EDA, Leu, 3allyloxyPyrSa, PyrSaV3Butene, Az2, PyrS1, PyrSc72SMe3ROMe, PyrSc72RMe3SOMe, PyrSc7045RMe, PyrSc7045SMe, PyrSc73Me2, PyrSc7, PyrSaA3Butene, PyrSadA3Butene, Dap7Gly, Dap7Pent, DapAc7PDA, Dap7Abu, 4VinylPyrSa, PyrSadV3Butene, PyrSaSar3Butene, PyrSaNip3Butene, PyrSaPro3Butene, PyrSa4VinMe2PhAc, or 3allylPyrSa. In some embodiments, X¹¹ is PyrS2. In some embodiments, X¹¹ is Lys. In some embodiments, X¹¹ is 3Thi. In some embodiments, X¹¹ is Ala. In some embodiments, X¹¹ is Phe. In some embodiments, X¹¹ is S3MePyrSc7. In some embodiments, X¹¹ is R3MePyrSc7. In some embodiments, X¹¹ is S3iPrPyrSc7. In some embodiments, X¹¹ is R3iPrPyrSc7.

In some embodiments, X¹¹ is or comprises a residue of an amino acid or a moiety selected from Table A-I, Table A-II, Table A-III and Table A-IV.

Various types of amino acid residues can be used for X¹², e.g., a residue of an amino acid of formula A-I, A-II, A-III, A-IV, A-V, A-VI, etc. or a salt thereof in accordance with the present disclosure. In some embodiments, X¹² is —N(R^{a1})-L^{a1}-C(R^{a2})(R^{a3})-L^{a2}-C(O)—, wherein each variable is independently as described herein. In some embodiments, X² is —N(R^{a1})_C(R^{a2})(R^{a3})—C(O)—, wherein each variable is independently as described herein. In some embodiments, X¹² is —N(R^{a1})—C(R^{a2})H—C(O)—, wherein each variable is independently as described herein. In some embodiments, R^{a1} is —H. In some embodiments, R^{a3} is —H.

In some embodiments, X¹¹ comprises a side chain comprising an optionally substituted aromatic group. In some embodiments, X¹² is an aromatic amino acid residue as described herein. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one oxygen atom. In some embodiments, an aromatic group is optionally substituted 5-membered heteroaryl having one sulfur atom. In some embodiments, an aromatic group is optionally substituted 6-membered heteroaryl having 1-3 heteroatoms. In some embodiments, an aromatic group is optionally substituted 6-membered heteroaryl having 1 nitrogen atom. In some embodiments, an aromatic group is optionally substituted phenyl. In some embodiments, X² comprises a side chain which is or comprises an optionally substituted aromatic group, wherein each substituent of the aromatic group is independently selected from halogen, —OR, —R, —C(O)OH, —C(O)NH₂, —CN, or —NO₂, wherein each R is independently C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X¹² comprises a side chain which is or comprises an optionally substituted aromatic group, wherein each substituent of the aromatic group is independently halogen. In some embodiments, X² comprises a side chain which is or comprises two optionally substituted aromatic groups. In some embodiments, X¹ comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X¹² is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5-6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, X² is a residue of an amino acid selected from 3Thi, 2F3MeF, Phe, 2COOHF, 2ClF, 2FurA, 20MeF, 2MeF, 2BrF, 2CNF, 2N02F, 2PyrA, 3PyrA, 4PyrA, His, 1NapA, 2Thi, and 2cmbF. In some embodiments, X¹² is a residue of 3Thi, 2F3MeF, or Phe. In some embodiments, X¹² is a residue of 3Thi. In some embodiments, X² is a residue of 2F3MeF. In some embodiments, X¹² is a residue of Phe. In some embodiments, X² is a residue of 2COOHF. In some embodiments, X¹² is a residue of 2ClF. In some embodiments, X¹² is a residue of 2FurA. In some embodiments, X² is a residue of 20MeF. In some embodiments, X¹² is a residue of 2MeF. In some embodiments, X¹² is a residue of 2BrF. In some embodiments, X¹² is a residue of 2CNF. In some embodiments, X¹² is a residue of 2N02F. In some embodiments, X¹² is a residue of 2PyraA. In some embodiments, X² is a residue of 3PyrA. In some embodiments, X¹² is a residue of 4PyrA. In some embodiments, X² is a residue of His. In some embodiments, X¹² is a residue of 1NapA. In some embodiments, X¹² is a residue of 2Thi. In some embodiments, X¹² is a residue of 2cmbF. In some embodiments, 3Thi provides better properties and/or activities than, e.g., Phe.

In some embodiments, X² is a residue of an amino acid whose side chain is hydrophobic. Various hydrophobic amino acid residues described herein may be utilized for X¹², e.g., those described for X³, X⁷, etc. In some embodiments, X² is a residue of nLeu, CypA, Ala, Leu, hLeu, Npg, Cpa, Nva, Cba, ChA, Val, Ile, Chg, hnLeu, or OctG. In some embodiments, X² is a residue of nLeu or CypA. In some embodiments, X¹² is a residue of nLeu. In some embodiments, X¹² is a residue of CypA. In some embodiments, X¹² is a residue of Ala. In some embodiments, X² is a residue of Leu. In some embodiments, X¹² is a residue of hLeu. In some embodiments, X¹² is a residue of Npg. In some embodiments, X¹² is a residue of Cpa. In some embodiments, X¹² is a residue of Nva. In some embodiments, X¹² is a residue of Cba. In some embodiments, X² is a residue of ChA. In some embodiments, X² is a residue of Val. In some embodiments, X¹² is a residue of Ile. In some embodiments, X¹² is a residue of Chg. In some embodiments, X¹² is a residue of hnLeu. In some embodiments, X² is a residue of OctG.

In some embodiments, X² is a residue of amino acid that comprises an acidic or polar group. In some embodiments, X¹² is a residue of amino acid whose side chain comprises an acidic group, e.g., a —COOH group or a salt form thereof (e.g., a compound of formula A-IV, etc.). Various acidic amino acid residues described herein may be utilized for X², e.g., those described for X², X⁵, X⁶, etc. In some embodiments, X¹² is 2COOHF. In some embodiments, X¹² is a residue of amino acid whose side chain comprises a polar group. In some embodiments, X¹² is a residue of amino acid whose side chain comprises an amide group, e.g., —C(O)N(R′)₂ such as —CONH₂. For example, in some embodiments, X¹² is a residue of 2cbmF. Various other polar amino acid residues described herein may also be utilized for X².

In some embodiments, X² is a residue of an amino acid selected from 3Thi, 2F3MeF, Phe, nLeu, 2COOHF, CypA, 2ClF, Ala, Abu, Leu, hLeu, Npg, Cpa, Nva, Cba, ChA, 2FurA, 20MeF, 2MeF, 2BrF, 2CNF, 2N02F, 2PyrA, 3PyrA, 4PyrA, His, 1NapA, Val, Ile, Chg, DiethA, hnLeu, OctG, 2Thi, and 2cmbF.

In some embodiments, X² is 3Thi, Phe, 2F3MeF, PyrS2, 2ClF, hnLeu, BztA, 2Thi, 2MeF, 2FF, 34ClF, Lys, nLeu, 2COOHF, 2PhF, hCbA, hCypA, hCha, CypA, hPhe, DipA, HepG, Dap7Abu, hhLeu, hhSer, HexG, [2IAPAc]2NH2F, Ala, Abu, Leu, hLeu, Npg, Cpa, PyrS1, [Bnc]2NH2F, [Phc]2NH2F, [BiPh]2NH2F, [3PyAc]2NH2F, Nva, Cba, ChA, 2FurA, 20MeF, 2BrF, 2CNF, 2N02F, 2PyrA, 3PyrA, 4PyrA, His, 1NapA, Val, Ile, Chg, DiethA, OctG, 2cbmF, c6Phe, [MePipAc]2NH2F, or [2PyCypCO]2NH2F. In some embodiments, X² is 3Thi. In some embodiments, X¹² is Phe. In some embodiments, X² is 3F3MeF. In some embodiments, X¹² is PyrS2. In some embodiments, X¹² is 2ClF. In some embodiments, X¹² is hnLeu. In some embodiments, X¹² is BztA. In some embodiments, X¹² is 2Thi. In some embodiments, X¹² is 2MeF. In some embodiments, X¹² is 2FF. In some embodiments, X¹² is 34ClF. In some embodiments, X¹² is 2NH2F. In some embodiments, X¹² is Trp. In some embodiments, X² is 5ClW. In some embodiments, X² is 6ClW. In some embodiments, X¹² is 2NH2F. In some embodiments, X¹² is [124TriAc]2NH2F. In some embodiments, X¹² is [124TriPr]2NH2F. In some embodiments, X¹² is [6QuiAc]2NH2F. In some embodiments, X¹² is [2PyAc]2NH2F. In some embodiments, X¹² is [2PyPrpc]2NH2F. In some embodiments, X¹² is [3PyPrpc]2NH2F. In some embodiments, X¹² is [4PyPrpc]2NH2F. In some embodiments, X¹² is [MeOPr]2NH2F. In some embodiments, X¹² is [PhOPr]2NH2F. In some embodiments, X¹² is [Me2MeOPr]2NH2F. In some embodiments, X¹² is [Me2NAc]2NH2F. In some embodiments, X¹² is [Me2NPr]2NH2F. In some embodiments, X¹ is [NdiMeButC]2NH2F. In some embodiments, X¹² is [3IAPAc]2NH2F. In some embodiments, X² is [15PyraPy]2NH2F. In some embodiments, X¹² is [MorphAc]2NH2F. In some embodiments, X¹² is [Nic]2NH2F. In some embodiments, X² is [2PyzCO]2NH2F. In some embodiments, X¹² is [5pymCO]2NH2F. In some embodiments, X¹² is [3FPyr2c]2NH2F. In some embodiments, X¹² is [4FPyr3c]2NH2F.

In some embodiments, X² is an amino acid residue for stapling as described herein. In some embodiments, X¹² is stapled, e.g., with X⁵. In some embodiments, X¹² is PyrS1. In some embodiments, X¹² is PyrS2.

In some embodiments, X² is or comprises a residue of an amino acid or a moiety selected from Table A-IV.

In some embodiments, X² interacts with Trp383 of beta-catenin or an amino acid residue corresponding thereto. In some embodiments, X¹² interacts with Asn415 of beta-catenin or an amino acid residue corresponding thereto. In some embodiments, X² interacts with Trp383 and Asn415 of beta-catenin or amino acid residues corresponding thereto.

Various types of amino acid residues can be used for X¹³, e.g., a residue of an amino acid of formula A-I, A-II, A-III, A-IV, A-V, A-VI, etc. or a salt thereof in accordance with the present disclosure. In some embodiments, X¹³ is —N(R^{a1})-L^{a1}-C(R^{a2})(R^{a3})-L^{a2}-C(O)—, wherein each variable is independently as described herein. In some embodiments, X¹³ is —N(R^{a1})—C(R^{a2})(R^{a3})—C(O)—, wherein each variable is independently as described herein. In some embodiments, X¹³ is —N(R^{a1})—C(R^{a2})H—C(O)—, wherein each variable is independently as described herein. In some embodiments, R^{a1} is —H. In some embodiments, R^{a3} is —H.

In some embodiments, X¹³ comprises a side chain which is or comprises an optionally substituted aromatic group. In some embodiments, X¹³ is an aromatic amino acid residue as described herein.

In some embodiments, X¹³ comprises a side chain comprising an optionally substituted aromatic group. In some embodiments, X¹³ is an aromatic amino acid residue as described herein. In some embodiments, an aromatic group is optionally substituted 5-membered heteroaryl having 1-3 heteroatoms. In some embodiments, an aromatic group is optionally substituted 5-membered heteroaryl having 1-3 nitrogen atoms. In some embodiments, an aromatic group is optionally substituted 5-membered heteroaryl having one sulfur atom. In some embodiments, an aromatic group is optionally substituted phenyl. In some embodiments, X¹³ comprises a side chain which is or comprises an optionally substituted aromatic group, wherein each substituent of the aromatic group is independently selected from halogen, —OR, —R, —C(O)OH, or —CN, wherein each R is independently hydrogen or C₁₋₄ alkyl or haloalkyl. In some embodiments, an aromatic group is phenyl. In some embodiments, an aromatic group is optionally substituted 8-10 membered bicyclic aryl or heteroaryl having 1-5 heteroatoms. In some embodiments, X¹³ comprises a side chain which is or comprises an optionally substituted aromatic group, wherein each substituent of the aromatic group is independently halogen. In some embodiments, X¹³ comprises a side chain which is or comprises two optionally substituted aromatic groups. In some embodiments, X¹³ comprises a side chain which is or comprises an optionally substituted aromatic group, wherein each substituent of the aromatic group is independently selected from halogen or —OH. In some embodiments, an aromatic group is phenyl. In some embodiments, an aromatic group is optionally substituted 8-10 membered bicyclic aryl or heteroaryl having 0-5 heteroatoms. In some embodiments, an aromatic group is optionally substituted 9-10 membered bicyclic aryl or heteroaryl having one heteroatom. In some embodiments, X¹³ is a residue of an amino acid of formula A-I or a salt thereof. In some embodiments, an amino acid residue has the structure of —NH—C(R^{a2})(R^{a3})—C(O)— or a salt thereof. In some embodiments, an amino acid residue has the structure of —NH—CH(R^{a3})—C)O)— or a salt thereof. As described herein, R^{a3} is -L^{a}-R′ wherein each variable is independently as described herein. In some embodiments, R′ is R as described herein. In some embodiments, R is an optionally substituted group selected from phenyl, 10-membered bicyclic aryl, 5-6 membered heteroaryl having 1-4 heteroatoms, and 9-10 membered bicyclic heteroaryl having 1-5 heteroatoms. In some embodiments, each substituent is independently halogen or —OH or C₁₋₆ haloaliphatic. In some embodiments, each substituent is independently halogen or —OH. In some embodiments, R is optionally substituted phenyl. In some embodiments, R is phenyl. In some embodiments, R is optionally substituted aryl. In some embodiments, R is aryl. In some embodiments, R is optionally substituted 5-membered heteroaryl having 1-4 heteroatoms. In some embodiments, R is optionally substituted 5-membered heteroaryl having 1 heteroatom. In some embodiments, optionally substituted R is 6-membered heteroaryl having 1-4 heteroatoms. In some embodiments, optionally substituted R is 6-membered heteroaryl having 1 heteroatom. In some embodiments, R is optionally substituted 9-membered heteroaryl having 1-5 heteroatoms. In some embodiments, R is optionally substituted 9-membered heteroaryl having 1 heteroatom. In some embodiments, R is optionally substituted 10-membered heteroaryl having 1-5 heteroatoms. In some embodiments, R is optionally substituted 10-membered heteroaryl having 1 heteroatom. In some embodiments, a heteroatom is nitrogen. In some embodiments, a heteroatom is oxygen. In some embodiments, a heteroatom is sulfur. As described herein, L^{a} is L. In some embodiments, L is a covalent bond. In some embodiments, L is an optionally substituted bivalent linear or branched C₁₋₁₀ hydrocarbon chain. In some embodiments, L is an optionally substituted bivalent linear C₁₋₁₀ hydrocarbon chain. In some embodiments, L is a bivalent linear or branched C₁₋₁₀ hydrocarbon chain. In some embodiments, L is a bivalent linear C₁₋₁₀ hydrocarbon chain. In some embodiments, L is optionally substituted —(CH₂)n-, wherein n is 1-10. In some embodiments, L is —(CH₂)n-, wherein n is 1-10. In some embodiments, L is —CH₂—. In some embodiments, L is —(CH₂)₂—. In some embodiments, L is —(CH₂)₃—. In some embodiments, L is —(CH₂)₄—. In some embodiments, L is an optionally substituted bivalent linear or branched C₁₋₁₀ hydrocarbon chain wherein one or more methylene units of L are independently replaced with —C(R′)₂—, —C(O)—, —N(R′)—, -Cy- or —O—. In some embodiments, L is an optionally substituted bivalent linear C₁₋₁₀ hydrocarbon chain wherein one or more methylene units of L are independently replaced with —C(R′)₂—, —C(O)—, —N(R′)—, -Cy- or —O—.

In some embodiments, X¹³ is a residue of BztA, 34ClF, or 2NapA. In some embodiments, X¹³ is a residue of BztA. In some embodiments, X¹³ is a residue of 34ClF. In some embodiments, X¹³ is a residue of 2NapA. In some embodiments, X¹³ is a residue of 3BrF. In some embodiments, X¹³ is a residue of 3Thi. In some embodiments, X¹³ is a residue of 34MeF.

In some embodiments, X¹³ is BztA, 34ClF, 3Thi, Phe, GlnR, 34MeF, 2NapA, Lys, PyrS2, 3BrF, 7FBztA, 2BrF, 3F3MeF, 4F3MeF, RbMe2NapA, RbMeBzta, SbMeBzta, 5IndA, 7ClBztA, 7MeBztA, Leu, 2ClF, 3ClF, 4BrF, 4ClF, or 3MeF. In some embodiments, X¹³ is BztA. In some embodiments, X¹³ is 34CIF. In some embodiments, X¹³ is 3Thi. In some embodiments, X¹³ is Phe. In some embodiments, X¹³ is GlnR. In some embodiments, X¹³ is 34MeF. In some embodiments, X¹³ is 2NapA. In some embodiments, X¹³ is Lys. In some embodiments, BztA provides better properties and/or activities than, e.g., Trp.

In some embodiments, X¹³ is or comprises a residue of an amino acid or a moiety selected from Table A-IV.

In some embodiments, X¹³ interacts with Gln379 of beta-catenin or an amino acid residue corresponding thereto. In some embodiments, X¹³ interacts with Leu382 of beta-catenin or an amino acid residue corresponding thereto. In some embodiments, X¹³ interacts with Val416 of beta-catenin or an amino acid residue corresponding thereto. In some embodiments, X¹³ interacts with Asn415 of beta-catenin or an amino acid residue corresponding thereto. In some embodiments, X¹³ interacts with Trp383 of beta-catenin or an amino acid residue corresponding thereto. In some embodiments, X¹³ interacts with Gln379, Leu382, Val416, Asn415, and Trp383 of beta-catenin or amino acid residues corresponding thereto.

Various types of amino acid residues can be used for X¹⁴, e.g., a residue of an amino acid of formula A-I, A-II, A-III, A-IV, A-V, A-VI, etc. or a salt thereof in accordance with the present disclosure. In some embodiments, X¹⁴ is —N(R^{a1})-L^{a1}-C(R^{a2})(R^{a3})-L^{a2}-C(O)—, wherein each variable is independently as described herein. In some embodiments, X¹⁴ is —N(R^{a1})—C(R^{a2})(R^{a3})—C(O)—, wherein each variable is independently as described herein. In some embodiments, X¹⁴ is —N(R^{a1})—C(R^{a2})H—C(O)—, wherein each variable is independently as described herein. In some embodiments, R^{a1} is —H. In some embodiments, R^{a3} is —H.

In some embodiments, X¹⁴ is an amino acid residue suitable for stapling. In some embodiments, X¹⁴ is stapled. In some embodiments, X¹⁴ is stapled with X¹⁰ as described herein. In some embodiments, X¹⁴ is stapled with X⁷ as described herein.

In some embodiments, X¹⁴ is an amino acid residue suitable for stapling, e.g., those described for X⁷, X¹⁰, etc.

Various types of amino acid residues can be used for X¹⁴. In some embodiments, X¹⁴ is GlnR, Lys, sAla, Gln, Cys, TriAzLys, AsnR, hGlnR, 4PipA, sAbu, Orn, dGlnR, [4mampiperidine]GlnR, [39N2spiroundecane]GlnR, [29N2spiroundecane]GlnR, iPrLys, sCH2S, [diaminobutane]GlnR, or [4aminopiperidine]GlnR. In some embodiments, X¹⁴ is GlnR. In some embodiments, X¹⁴ is Lys. In some embodiments, X¹⁴ is sAla. In some embodiments, X¹⁴ is Gln. In some embodiments, X¹⁴ is Cys. In some embodiments, X¹⁴ is TriAzLys. In some embodiments, X¹⁴ is AsnR. In some embodiments, X¹⁴ is hGlnR. In some embodiments, X¹⁴ is 4PipA. In some embodiments, X¹⁴ is sAbu. In some embodiments, X¹⁴ is Orn. In some embodiments, X¹⁴ is dGlnR. In some embodiments, X¹⁴ is [4mampiperidine]GlnR. In some embodiments, X¹⁴ is [39N2spiroundecane]GlnR. In some embodiments, X¹⁴ is [29N2spiroundecane]GlnR. In some embodiments, X¹⁴ is iPrLys. In some embodiments, X¹⁴ is sCH2S. In some embodiments, X¹⁴ is [diaminobutane]GlnR. In some embodiments, X¹⁴ is [4aminopiperidine]GlnR.

In some embodiments, X¹⁴ is an aromatic amino acid residue as described herein. In some embodiments, X¹⁴ is BtzA.

In some embodiments, v14 is a polar amino acid residue as described herein. In some embodiments, X¹⁴ is Gln.

In some embodiments, X¹⁴ is a C-terminus amino acid residue. In some embodiments, X¹⁴ has a free —COOH or a salt form thereof. In some embodiments, —C(O)OH of X¹⁴ is capped. In some embodiments, —C(O)OH of X¹⁴ is converted into —C(O)N(R′)₂, wherein each R is independently as described herein. In some embodiments, —C(O)N(R′)₂ is —C(O)NHR′. In some embodiments, each R′ is independently R. In some embodiments, each R′ is —H. In some embodiments, R is H. In some embodiments, R is optionally substituted C₁₋₆ aliphatic. In some embodiments, R is optionally substituted C₁₋₆ alkyl. In some embodiments, R is ethyl. In some embodiments, R is

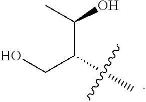

In some embodiments, R is —CH(CH₃)CH₂OH. In some embodiments, R is —(S)—CH(CH₃)CH₂OH. In some embodiments, R is —(R)—CH(CH₃)CH₂OH. In some embodiments, R is —CH(CH₂OH)₂.

In some embodiments, two R′ groups are taken together with the nitrogen atom to which they are attached to form a ring as described herein. In some embodiments, —N(R′)₂ is

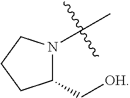 [SMILES: *N1CCC[C@H]1CO]

In some embodiments, X¹⁴ is GlnR, BztA, sAla, 34ClF, Cys, Ala, Lys, AsnR, aMeC, PyrS2, Gln, hGlnR, 3Thi, Lys, Pen, GlnR, TriAzLys, hCys, 4PipA, sAbu, Orn, 1MeK, [4mampiperidine]GlnR, [39N2spiroundecane]GlnR, [29N2spiroundecane]GlnR, iPrLys, sCH2S, AsnEDA, AsnS3APyr, [diaminobutane]GlnR, [4aminopiperidine]GlnR, dGlnR, GlnEDA, AsnPpz, GlnPpz, GlnR3APyr, GlnS3APyr, GlnMe2EDA, AsnMe2EDA, AsnMeEDA, AsnR3APyr. In some embodiments, X¹⁴ is GlnR. In some embodiments, X¹⁴ is BztA. In some embodiments, X¹⁴ is sAla. In some embodiments, X¹⁴ is 34ClF. In some embodiments, X¹⁴ is Cys. In some embodiments, X¹⁴ is Ala. In some embodiments, X¹⁴ is Lys. In some embodiments, X¹⁴ is AsnR. In some embodiments, X¹⁴ is aMeC. In some embodiments, X¹⁴ is PyrS2. In some embodiments, X¹⁴ comprises a C-terminal group, e.g., —NH2. In some embodiments, X¹⁴ is Gln. In some embodiments, X¹⁴ is hGlnR. In some embodiments, X¹⁴ is 3Thi. In some embodiments, X¹⁴ is Lys. In some embodiments, X¹⁴ is GlnR*3. In some embodiments, X¹⁴ is dLys. In some embodiments, X¹⁴ is GlnMePDA. In some embodiments, X¹⁴ is GlnT4CyMe. In some embodiments, X¹⁴ is GlnMeBDA. In some embodiments, X¹⁴ is Gln5DA. In some embodiments, X¹⁴ is Gln6DA. In some embodiments, X¹⁴ is TriAzOm. In some embodiments, X¹⁴ is Phe. In some embodiments, X¹⁴ is GlnC4CyMe. In some embodiments, X¹⁴ is Gln3ACPip. In some embodiments, X¹⁴ is GlnPipAz. In some embodiments, X¹⁴ is GlnPip4AE. In some embodiments, X¹⁴ forms intramolecular hydrogen bonding.

In some embodiments, X¹⁴ is or comprises a residue of an amino acid or a moiety selected from Table A-I, Table A-II, Table A-III and Table A-IV.

In some embodiments, p15 is 1. In some embodiments, p15 is 0.

Various types of amino acid residues can be used for X⁵, e.g., a residue of an amino acid of formula A-I, A-II, A-III, A-IV, A-V, A-VI, etc. or a salt thereof in accordance with the present disclosure. In some embodiments, X⁵ is —N(R^{a1})-L^{a1}-C(R^{a2})(R^{a3})-L^{a2}-C(O)—, wherein each variable is independently as described herein. In some embodiments, X⁵ is —N(R^{a1})—C(R^{a2})(R^{a3})—C(O)—, wherein each variable is independently as described herein. In some embodiments, X⁵ is —N(R^{a1})—C(R^{a2})H—C(O)—, wherein each variable is independently as described herein. In some embodiments, R^{a1} is —H. In some embodiments, R^{a3} is —H.

Various types of amino acid residues can be used for X⁵. In some embodiments, X¹⁵ is a residue of Ala, Leu, Val, Aib, MorphNva, Thr, dAla, dLeu, [BiotinPEG8]Lys, Glu, or AzLys.

In some embodiments, X⁵ is or comprises a label, e.g., a label for detection, binding, etc. In some embodiments, a label is or comprises biotin. In some embodiments, X⁵ is [BiotinPEG8]Lys.

In some embodiments, X⁵ is a hydrophobic amino acid residue as described herein, e.g., those described for X³, X⁸, etc. In some embodiments, X⁵ is Ala. In some embodiments, X⁵ is Leu. In some embodiments, X⁵ is Val. In some embodiments, X⁵ is Aib. In some embodiments, X⁵ is dAla. In some embodiments, X⁵ is dLeu.

In some embodiments, X⁵ is an amino acid residue whose side chain comprises an amino group. In some embodiments, X⁵ is MorphNva.

In some embodiments, X⁵ is an amino acid residue suitable for stapling as described herein. In some embodiments, X⁵ is GlnR. In some embodiments, it is stapled with X¹¹. In some embodiments, X¹¹ is Lys.

In some embodiments, X⁵ is a polar amino acid residue as described herein, e.g., those described for X², X⁵, X⁶, etc. In some embodiments, X⁵ is Thr. In some embodiments, X⁵ is —Ser.

In some embodiments, X⁵ is an acidic amino acid residue as described herein, e.g., those described for X², X⁵, X⁶, etc. In some embodiments, X⁵ is Glu.

In some embodiments, X⁵ is a C-terminus amino acid residue. In some embodiments, X¹⁵ has a free —COOH or a salt form thereof. In some embodiments, —C(O)OH of X⁵ is capped. In some embodiments, —C(O)OH of X⁵ is converted into —C(O)N(R′)₂, wherein each R is independently as described herein. In some embodiments, —C(O)N(R′)₂ is —C(O)NHR′. In some embodiments, each R′ is independently R. In some embodiments, each R′ is —H. In some embodiments, R is H. In some embodiments, R is optionally substituted C₁₋₆ aliphatic. In some embodiments, R is optionally substituted C₁₋₆ alkyl. In some embodiments, R is ethyl. In some embodiments, R is

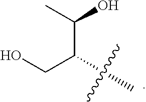

In some embodiments, R is —CH(CH₃)CH₂OH. In some embodiments, R is —(S)—CH(CH₃)CH₂OH. In some embodiments, R is —(R)—CH(CH₃)CH₂OH. In some embodiments, R is —CH(CH₂OH)₂.

In some embodiments, an agent comprises a C-terminal group. In some embodiments, a C-terminal group is —OH. In some embodiments, a C-terminal group is —NH₂.

In some embodiments, X⁵ is Ala, GlnR, Leu, Val, Ser, Thr, 3Thi, BztA, Aib, MorphNva, dAla, dLeu, Pro, Phe, [BiotinPEG8]Lys, Throl, Glu, AzLys, Npg, Trp, Tyr, Lys, Prool, Alaol, Gly, dPro, Asn, Gln, Ala_D3, [mPEG4]Lys, [mPEG8]Lys, [mPEG16]Lys. In some embodiments, X¹⁵ is Ala. In some embodiments, X⁵ comprises a C-terminal group, e.g., —NH₂. In some embodiments, X⁵ is GlnR. In some embodiments, X⁵ is Leu. In some embodiments, X⁵ is Val. In some embodiments, X⁵ is Ser. In some embodiments, X⁵ is Thr. In some embodiments, X⁵ is 3Thi. In some embodiments, X⁵ is BztA. In some embodiments, X⁵ is [mPEG37]-Lys. In some embodiments, X⁵ is dVal. In some embodiments, X⁵ is 34ClF.

In some embodiments, X⁵ is or comprises a residue of an amino acid or a moiety selected from Table A-IV.

In some embodiments, p16 is 1. In some embodiments, p16 is 0.

Various types of amino acid residues can be used for X¹⁶, e.g., a residue of an amino acid of formula A-I, A-II, A-III, A-IV, A-V, A-VI, etc. or a salt thereof in accordance with the present disclosure. In some embodiments, X¹⁶ is —N(R^{a1})-L^{a1}-C(R^{a2})(R^{a3})-L^{a2}-C(O)—, wherein each variable is independently as described herein. In some embodiments, X¹⁶ is —N(R^{a1})—C(R^{a2})(R^{a3})—C(O)—, wherein each variable is independently as described herein. In some embodiments, X¹⁶ is —N(R^{a1})—C(R^{a2})H—C(O)—, wherein each variable is independently as described herein. In some embodiments, R^{a1} is —H. In some embodiments, R^{a3} is —H.

Various types of amino acid residues can be used for X¹⁶. In some embodiments, X¹⁶ is a residue of Ser, Ala, Glu, Aib, Asp, Thr, or aThr.

In some embodiments, X¹⁶ is a polar amino acid residue as described herein, e.g., those described for X², X⁵, X⁶, etc. In some embodiments, X¹⁶ is Thr. In some embodiments, X¹⁶ is —Ser. In some embodiments, X¹⁶ is aThr.

In some embodiments, X¹⁶ is a hydrophobic amino acid residue as described herein, e.g., those described for X³, X⁸, etc. In some embodiments, X¹⁶ is Ala. In some embodiments, X¹⁶ is Leu. In some embodiments, X¹⁶ is Val. In some embodiments, X¹⁶ is Aib. In some embodiments, X¹⁶ is dAla. In some embodiments, X¹⁶ is dLeu.

In some embodiments, X¹⁶ is an acidic amino acid residue as described herein, e.g., those described for X², X⁵, X⁶, etc. In some embodiments, X¹⁶ is Glu. In some embodiments, X¹⁶ is Asp.

In some embodiments, X¹⁶ is Ala, Ser, Glu, GlnR, BztA, Thr, Aib, Asp, Lys, aThr, Val, or Arg. In some embodiments, X¹⁶ comprises a C-terminal group, e.g., NH₂, OH, Serol, NHEt, NHMe, dAlaol, etc.

In some embodiments, X¹⁶ is or comprises a residue of an amino acid or a moiety selected from Table A-IV.

In some embodiments, p17 is 1. In some embodiments, p17 is 0.

Various types of amino acid residues can be used for X¹⁷, e.g., a residue of an amino acid of formula A-I, A-II, A-III, A-IV, A-V, A-VI, etc. or a salt thereof in accordance with the present disclosure. In some embodiments, X¹⁷ is —N(R^{a1})-L^{a1}-C(R^{a2})(R^{a1})-L^{a2}-C(O)—, wherein each variable is independently as described herein. In some embodiments, X¹⁷ is —N(R^{a1})—C(R^{a2})(R^{a3})—C(O)—, wherein each variable is independently as described herein. In some embodiments, X¹⁷ is —N(R^{a1})—C(R^{a2})H—C(O)—, wherein each variable is independently as described herein. In some embodiments, R^{a1} is —H. In some embodiments, R^{a3} is —H.

In some embodiments, X¹⁷ is a hydrophobic amino acid residue as described herein, e.g., those described for X³, X⁸, etc. In some embodiments, X¹⁷ is a residue of Ala or Leu. In some embodiments, X¹⁷ is a residue of Ala. In some embodiments, X¹⁷ is a residue of Leu.

In some embodiments, X¹⁷ is Ala, Leu, GlnR, GlnR, Pro, Thr, Val, Lys, Arg, [Ac] Lys, [mPEG4]Lys, [mPEG8]Lys, or [mPEG16]Lys. In some embodiments, X¹⁷ comprises a C-terminal group, e.g., NH₂, NHEt, OH, etc. In some embodiments, X¹⁷ is [Ac-dPEG2]-Lys. In some embodiments, X¹⁷ is [Ac-PEG8]-Lys. In some embodiments, X¹⁷ is [Oct-dPEG2]-Lys. In some embodiments, X¹⁷ is [Oct-PEG8]-Lys. In some embodiments, X¹⁷ is [C18-dPEG2]-Lys. In some embodiments, X¹⁷ is [C18-PEG8]-Lys. In some embodiments, X¹⁷ is [AdamantC-dPEG2]-Lys. In some embodiments, X¹⁷ is [AdamantC-PEG8]-Lys. In some embodiments, X¹⁷ is [lithocholate-dPEG2]-Lys. In some embodiments, X¹⁷ is [lithocholate-PEG8]-Lys.

In some embodiments, X¹⁷ is or comprises a residue of an amino acid or a moiety selected from Table A-IV.

In some embodiments, X¹⁷ comprises a polar side chain. In some embodiments, it is a polar amino acid residue as described herein. In some embodiments, X¹⁷ comprises a non-polar side chain. In some embodiments, X¹⁷ comprises a hydrophobic side chain. In some embodiments, it is a hydrophobic amino acid residue as described herein. In some embodiments, X¹⁷ comprises an aliphatic side chain. In some embodiments, X¹⁷ comprises an alkyl side chain. In some embodiments, a side chain of X¹⁷ is C₁₋₁₀ alkyl. In some embodiments, X¹⁷ comprises a side chain comprising an optionally substituted aromatic group. In some embodiments, it is an aromatic amino acid residue as described herein. In some embodiments, X¹⁷ comprises a side chain comprising an acidic group, e.g., —COOH. In some embodiments, it is an acidic amino acid residue as described herein. In some embodiments, X¹⁷ comprises a side chain comprising a basic group, e.g., —N(R)₂. In some embodiments, it is a basic amino acid residue as described herein. In some embodiments, X¹⁷ comprises a detectable moiety such as a fluorescent moiety. In some embodiments, X¹⁷ is Ala, dAla, or Leu. In some embodiments, X¹⁷ is Ala. In some embodiments, X¹⁷ is dAla. In some embodiments, X¹⁷ is Leu.

In some embodiments, X¹⁷ is or comprises a residue of an amino acid or a moiety selected from Table A-IV.

In some embodiments, p17 is 1. In some embodiments, p17 is 0.

Various types of amino acid residues can be used for X¹⁸, e.g., a residue of an amino acid of formula A-I, A-II, A-III, A-IV, A-V, A-VI, etc. or a salt thereof in accordance with the present disclosure. In some embodiments, X¹⁸ is —N(R^{a1})-L^{a1}-C(R^{a2})(R^{a3})-L^{a2}-C(O)—, wherein each variable is independently as described herein. In some embodiments, X¹⁵ is —N(R^{a1})—C(R^{a2})(R^{a3})—C(O)—, wherein each variable is independently as described herein. In some embodiments, X¹⁵ is —N(R^{a1})—C(R^{a2})H—C(O)—, wherein each variable is independently as described herein. In some embodiments, R^{a1} is —H. In some embodiments, R^{a3} is —H.

In some embodiments, X¹⁸ comprises a polar side chain. In some embodiments, it is a polar amino acid residue as described herein. In some embodiments, X¹⁸ comprises a non-polar side chain. In some embodiments, X¹⁸ comprises a hydrophobic side chain. In some embodiments, it is a hydrophobic amino acid residue as described herein. In some embodiments, X¹⁸ comprises an aliphatic side chain. In some embodiments, X¹⁸ comprises an alkyl side chain. In some embodiments, a side chain of X¹⁸ is C₁₋₁₀ alkyl. In some embodiments, X¹⁸ comprises a side chain comprising an optionally substituted aromatic group. In some embodiments, it is an aromatic amino acid residue as described herein. In some embodiments, X¹⁸ comprises a side chain comprising an acidic group, e.g., —COOH. In some embodiments, it is an acidic amino acid residue as described herein. In some embodiments, X¹⁸ comprises a side chain comprising a basic group, e.g., —N(R)₂. In some embodiments, it is a basic amino acid residue as described herein. In some embodiments, X¹⁸ comprises a detectable moiety such as a fluorescent moiety. In some embodiments, X¹⁸ is Aib, Ala, or Leu. In some embodiments, X^{1s} is Ala or Leu. In some embodiments, X's is Aib. In some embodiments, X¹⁸ is Ala. In some embodiments, X^{1s} is Leu. In some embodiments, X^{1s} is Pro. In some embodiments, X¹⁸ is [Ac] Lys. In some embodiments, X¹⁸ is [mPEG4]Lys. In some embodiments, X¹⁸ is [mPEG8]Lys. In some embodiments, X¹⁸ is [mPEG16]Lys. In some embodiments, X¹⁸ is Thr. In some embodiments, X¹⁸ is GlnR. In some embodiments, X¹⁸ is [mPEG37]Lys. In some embodiments, X¹⁸ is [PEG4triPEG16]Lys. In some embodiments, X¹⁸ is [PEG4triPEG36]Lys. In some embodiments, X¹⁸ comprises a C-terminal group as described herein.

In some embodiments, X¹⁸ is or comprises a residue of an amino acid or a moiety selected from Table A-IV.

In some embodiments, p18 is 1. In some embodiments, p18 is 0.

Various types of amino acid residues can be used for X¹⁹, e.g., a residue of an amino acid of formula A-I, A-II, A-III, A-IV, A-V, A-VI, etc. or a salt thereof in accordance with the present disclosure. In some embodiments, X¹⁹ is —N(R^{a1})-L^{a1}-C(R^{a2})(R^{a3})-L^{a2}-C(O)—, wherein each variable is independently as described herein. In some embodiments, X¹⁹ is —N(R^{a1})—C(R^{a2})(R^{a3})—C(O)—, wherein each variable is independently as described herein. In some embodiments, X¹⁹ is —N(R^{a1})—C(R^{a2})H—C(O)—, wherein each variable is independently as described herein. In some embodiments, R^{a1} is —H. In some embodiments, R^{a3} is —H.

In some embodiments, X¹⁹ comprises a polar side chain. In some embodiments, it is a polar amino acid residue as described herein. In some embodiments, X¹⁹ comprises a non-polar side chain. In some embodiments, X¹⁹ comprises a hydrophobic side chain. In some embodiments, it is a hydrophobic amino acid residue as described herein. In some embodiments, X¹⁹ comprises an aliphatic side chain. In some embodiments, X¹⁹ comprises an alkyl side chain. In some embodiments, a side chain of X¹⁹ is C₁₋₁₀ alkyl. In some embodiments, X¹⁹ comprises a side chain comprising an optionally substituted aromatic group. In some embodiments, it is an aromatic amino acid residue as described herein. In some embodiments, X¹⁹ comprises a side chain comprising an acidic group, e.g., —COOH. In some embodiments, it is an acidic amino acid residue as described herein. In some embodiments, X¹⁹ comprises a side chain comprising a basic group, e.g., —N(R)₂. In some embodiments, it is a basic amino acid residue as described herein. In some embodiments, X¹⁹ comprises a detectable moiety such as a fluorescent moiety. In some embodiments, X¹⁹ is Aib, Ala, or Leu. In some embodiments, X¹⁹ is Ala or Leu. In some embodiments, X¹⁹ is Aib. In some embodiments, X¹⁹ is Ala. In some embodiments, X¹⁹ is Leu. In some embodiments, X¹⁹ is Thr. In some embodiments, X¹⁹ is Val. In some embodiments, X¹⁹ is Pro.

In some embodiments, X¹⁹ is or comprises a residue of an amino acid or a moiety selected from Table A-IV.

In some embodiments, p19 is 1. In some embodiments, p19 is 0.

Various types of amino acid residues can be used for X²⁰, e.g., a residue of an amino acid of formula A-I, A-II, A-III, A-IV, A-V, A-VI, etc. or a salt thereof in accordance with the present disclosure. In some embodiments, X²⁰ is —N(R^{a1})-L^{a1}-C(R^{a2})(R^{a3})-L^{a2}-C(O)—, wherein each variable is independently as described herein. In some embodiments, X²⁰ is —N(R^{a1})—C(R^{a2})(R^{a3})—C(O)—, wherein each variable is independently as described herein. In some embodiments, X²⁰ is —N(R^{a1})—C(R^{a2})H—C(O)—, wherein each variable is independently as described herein. In some embodiments, R^{a1} is —H. In some embodiments, R^{a3} is —H.

In some embodiments, X²⁰ comprises a polar side chain. In some embodiments, it is a polar amino acid residue as described herein. In some embodiments, X²⁰ comprises a non-polar side chain. In some embodiments, X²⁰ comprises a hydrophobic side chain. In some embodiments, it is a hydrophobic amino acid residue as described herein. In some embodiments, X²⁰ comprises an aliphatic side chain. In some embodiments, X²⁰ comprises an alkyl side chain. In some embodiments, a side chain of X²⁰ is C₁₋₁₀ alkyl. In some embodiments, X²⁰ comprises a side chain comprising an optionally substituted aromatic group. In some embodiments, it is an aromatic amino acid residue as described herein. In some embodiments, X²⁰ comprises a side chain comprising an acidic group, e.g., —COOH. In some embodiments, it is an acidic amino acid residue as described herein. In some embodiments, X²⁰ comprises a side chain comprising a basic group, e.g., —N(R)₂. In some embodiments, it is a basic amino acid residue as described herein. In some embodiments, X²⁰ comprises a detectable moiety such as a fluorescent moiety. In some embodiments, X²⁰ is Aib, Ala, or Leu. In some embodiments, X²⁰ is Ala or Leu. In some embodiments, X²⁰ is Aib. In some embodiments, X²⁰ is Ala. In some embodiments, X²⁰ is Leu. In some embodiments, X²⁰ is Lys. In some embodiments, X²⁰ is nLeu. In some embodiments, X²⁰ is Val. In some embodiments, X²⁰ is Arg.

In some embodiments, X²⁰ is or comprises a residue of an amino acid or a moiety selected from Table A-IV.

In some embodiments, p20 is 1. In some embodiments, p20 is 0.

Various types of amino acid residues can be used for X²¹, e.g., a residue of an amino acid of formula A-I, A-II, A-III, A-IV, A-V, A-VI, etc. or a salt thereof in accordance with the present disclosure. In some embodiments, X²¹ is —N(R^{a1})-L^{a1}-C(R^{a2})(R^{a3})-L^{a2}-C(O)—, wherein each variable is independently as described herein. In some embodiments, X²¹ is —N(R^{a1})_C(R^{a2})(R^{a3})—C(O)—, wherein each variable is independently as described herein. In some embodiments, X²¹ is —N(R^{a1})—C(R^{a2})H—C(O)—, wherein each variable is independently as described herein. In some embodiments, R^{a1} is —H. In some embodiments, R^{a3} is —H.

In some embodiments, X²¹ comprises a polar side chain. In some embodiments, it is a polar amino acid residue as described herein. In some embodiments, X²¹ comprises a non-polar side chain. In some embodiments, X²¹ comprises a hydrophobic side chain. In some embodiments, it is a hydrophobic amino acid residue as described herein. In some embodiments, X²¹ comprises an aliphatic side chain. In some embodiments, X²¹ comprises an alkyl side chain. In some embodiments, a side chain of X² is C₁₋₁₀ alkyl. In some embodiments, X²¹ comprises a side chain comprising an optionally substituted aromatic group. In some embodiments, it is an aromatic amino acid residue as described herein. In some embodiments, X²¹ comprises a side chain comprising an acidic group, e.g., —COOH. In some embodiments, it is an acidic amino acid residue as described herein. In some embodiments, X²¹ comprises a side chain comprising a basic group, e.g., —N(R)₂. In some embodiments, it is a basic amino acid residue as described herein. In some embodiments, X²¹ comprises a detectable moiety such as a fluorescent moiety. In some embodiments, X²¹ is Aib, Ala, or Leu. In some embodiments, X²¹ is Ala or Leu. In some embodiments, X²¹ is Aib. In some embodiments, X²¹ is Ala. In some embodiments, X²¹ is Leu. In some embodiments, X²¹ is Lys. In some embodiments, X²¹ is nLeu. In some embodiments, X²¹ is Arg.

In some embodiments, X²¹ is or comprises a residue of an amino acid or a moiety selected from Table A-IV.

In some embodiments, p21 is 1. In some embodiments, p21 is 0.

Various types of amino acid residues can be used for X²², e.g., a residue of an amino acid of formula A-I, A-II, A-III, A-IV, A-V, A-VI, etc. or a salt thereof in accordance with the present disclosure. In some embodiments, X²² is —N(R^{a1})-L^{a1}-C(R^{a2})(R^{a3})-L^{a2}-C(O)—, wherein each variable is independently as described herein. In some embodiments, X²² is —N(R^{a1})—C(R^{a2})(R^{a3})—C(O)—, wherein each variable is independently as described herein. In some embodiments, X²² is —N(R^{a1})—C(R^{a2})H—C(O)—, wherein each variable is independently as described herein. In some embodiments, R^{a1} is —H. In some embodiments, R^{a3} is —H.

In some embodiments, X²² comprises a polar side chain. In some embodiments, it is a polar amino acid residue as described herein. In some embodiments, X²² comprises a non-polar side chain. In some embodiments, X²² comprises a hydrophobic side chain. In some embodiments, it is a hydrophobic amino acid residue as described herein. In some embodiments, X²² comprises an aliphatic side chain. In some embodiments, X²² comprises an alkyl side chain. In some embodiments, a side chain of X²² is C₁₋₁₀ alkyl. In some embodiments, X²² comprises a side chain comprising an optionally substituted aromatic group. In some embodiments, it is an aromatic amino acid residue as described herein. In some embodiments, X²² comprises a side chain comprising an acidic group, e.g., —COOH. In some embodiments, it is an acidic amino acid residue as described herein. In some embodiments, X²² comprises a side chain comprising a basic group, e.g., —N(R)₂. In some embodiments, it is a basic amino acid residue as described herein. In some embodiments, X²² comprises a detectable moiety such as a fluorescent moiety. In some embodiments, X²² is Aib, Ala, or Leu. In some embodiments, X²² is Ala or Leu. In some embodiments, X²² is Aib. In some embodiments, X²² is Ala. In some embodiments, X²² is Leu. In some embodiments, X²² is Lys.

In some embodiments, X²² is or comprises a residue of an amino acid or a moiety selected from Table A-IV.

In some embodiments, p22 is 1. In some embodiments, p22 is 0.

Various types of amino acid residues can be used for X²³, e.g., a residue of an amino acid of formula A-I, A-II, A-III, A-IV, A-V, A-VI, etc. or a salt thereof in accordance with the present disclosure. In some embodiments, X²³ is —N(R^{a1})-L^{a1}-C(R^{a2})(R^{a3})-L^{a2}-C(O)—, wherein each variable is independently as described herein. In some embodiments, X²³ is —N(R^{a1})—C(R^{a2})(R^{a3})—C(O)—, wherein each variable is independently as described herein. In some embodiments, X²³ is —N(R^{a1})—C(R^{a2})H—C(O)—, wherein each variable is independently as described herein. In some embodiments, R^{a1} is —H. In some embodiments, R^{a3} is —H.

In some embodiments, X²³ comprises a polar side chain. In some embodiments, it is a polar amino acid residue as described herein. In some embodiments, X²³ comprises a non-polar side chain. In some embodiments, X²³ comprises a hydrophobic side chain. In some embodiments, it is a hydrophobic amino acid residue as described herein. In some embodiments, X²³ comprises an aliphatic side chain. In some embodiments, X²³ comprises an alkyl side chain. In some embodiments, a side chain of X²³ is C₁₋₁₀ alkyl. In some embodiments, X²³ comprises a side chain comprising an optionally substituted aromatic group. In some embodiments, it is an aromatic amino acid residue as described herein. In some embodiments, X²³ comprises a side chain comprising an acidic group, e.g., —COOH. In some embodiments, it is an acidic amino acid residue as described herein. In some embodiments, X²³ comprises a side chain comprising a basic group, e.g., —N(R)₂. In some embodiments, it is a basic amino acid residue as described herein. In some embodiments, X²³ comprises a detectable moiety such as a fluorescent moiety. In some embodiments, X²³ is Aib, Ala, or Leu. In some embodiments, X²³ is Ala or Leu. In some embodiments, X²³ is Aib. In some embodiments, X²³ is Ala. In some embodiments, X²³ is Leu.

In some embodiments, X²³ is or comprises a residue of an amino acid or a moiety selected from Table A-IV.

In some embodiments, p23 is 1. In some embodiments, p23 is 0.

In some embodiments, an agent is or comprises a peptide having the structure of:

R^{N}—[X]ₚ—[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇—[X]ₚ—R^{C},

or a salt thereof, wherein:
each X is independently an amino acid residue;
each p and p′ is independently 0-10;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, p is 0. In some embodiments, p is 1, 2, 3, 4, 5, 6, 7, 8, 9, or 10. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p′ is 0. In some embodiments, p′ is 1, 2, 3, 4, 5, 6, 7, 8, 9, or 10. In some embodiments, p′ is 1. In some embodiments, p′ is 2. In some embodiments, p′ is 3. In some embodiments, p′ is 4. In some embodiments, p′ is 5. In some embodiments, p′ is 6. In some embodiments, p′ is 7. In some embodiments, p′ is 8. In some embodiments, p′ is 9. In some embodiments, p′ is 10.

In some embodiments, R^{N} is an N-terminus capping group. In some embodiments, R^{N} is —C(O)R, wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R^{N} is Ac. In some embodiments, R^{N} is a group suitable for stapling, or is stapled. In some embodiments, R^{N} is 4pentenyl. In some embodiments, R^{N} is 5hexenyl. In some embodiments, R^{N} is BzAm20Allyl. In some embodiments, R^{N} is Ac, NPyroR3, 5hexenyl, 4pentenyl, Bua, C3a, Cpc, Cbc, CypCO, Bnc, CF3CO, 2PyCypCO, 4THPCO, Isobutyryl, Ts, 15PyraPy, 2PyBu, 4PymCO, 4PyPrpc, 3IAPAc, 4MePipzPrpC, MePipAc, MeImid4SO2, BzAm20Allyl, Hex, 2PyzCO, 3Phc3, MeOPr, lithocholate, 2FPhc, PhC, MeSO2, Isovaleryl, EtHNCO, TzPyr, 8IAP, 3PydCO, 2PymCO, 5PymCO, 1Imidac, 2F2PyAc, 2IAPAc, 124TriPr, 6QuiAc, 3PyAc, 123TriAc, 1PyrazoleAc, 3PyPrpc, 5PymAc, 1PydoneAc, 124TriAc, Me2NAc, 8QuiSO2, mPEG4, mPEG8, mPEG16 or mPEG24.

In some embodiments, R^{C} is a C-terminus capping group. In some embodiments, R^{C} is —N(R′)₂ wherein each R′ is independently as described herein. In some embodiments, R^{C} is —NHR′ wherein R′ is as described herein. In some embodiments, R^{C} is —N(R)₂ wherein each R is independently as described herein. In some embodiments, R^{C} is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R is ethyl. In some embodiments, R^{C} is —NH₂. In some embodiments, R^{C} is —NHEt.

In some embodiments, R^{C} is —NHC(CH₃)CH₂OH. In some embodiments, R^{C} is —(S)—NHC(CH₃)CH₂OH. In some embodiments, R^{C} is —(R)—NHC(CH₃)CH₂OH. In some embodiments, R^{C} is

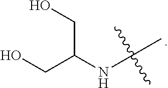 [SMILES: *NC(CO)CO]

In some embodiments, R^{C} is

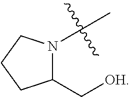 [SMILES: *N1CCCC1CO]

In some embodiments, R^{C} is

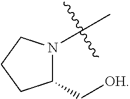 [SMILES: *N1CCC[C@H]1CO]

In some embodiments, R^{C} is

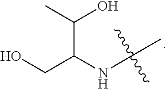 [SMILES: *NC(CO)C(C)O]

In some embodiments, R^{C} is

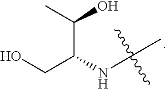 [SMILES: *N[C@H](CO)[C@@H](C)O]

In some embodiments, R^{C} is -Alaol, wherein the amino group of -Alaol is bonded to the last —C(O)— of the peptide backbone

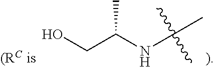 [SMILES: *N[C@@H](C)CO]

In some embodiments, R^{C} is -dAlaol, wherein the amino group of -dAlaol is bonded to the last —C(O)— of the peptide backbone

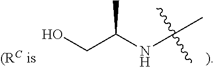 [SMILES: *N[C@H](C)CO]

In some embodiments, R^{C} is -Prool, wherein the amino group of -Prool is bonded to the last —C(O)— of the peptide backbone

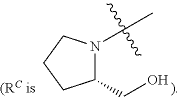 [SMILES: *N1CCC[C@H]1CO]

In some embodiments, R^{C} is -Throl, wherein the amino group of -Throl is bonded to the last —C(O)— of the peptide backbone

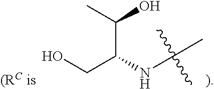 [SMILES: *N[C@H](CO)[C@@H](C)O]

In some embodiments, R^{C} is -Serol, wherein the amino group of -Serol is bonded to the last —C(O)— of the peptide backbone

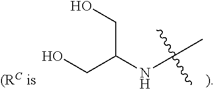 [SMILES: *NC(CO)CO]

In some embodiments, R^{C} is —OH.

### Amino Acids

As appreciated by those skilled in the art, various amino acids may be utilized in accordance with the present disclosure. For example, both naturally occurring and non-naturally occurring amino acids can be utilized in accordance with the present disclosure. In some embodiments, an amino acid is a compound comprising an amino group that can form an amide group with a carboxyl group and a carboxyl group. In some embodiments, an amino acid is an alpha amino acid. In some embodiments, an amino acid is a beta-amino acid. In some embodiments, an amino acid is a D-amino acid. In some embodiments, an amino acid is a L-amino acid. In some embodiments, an amino acid is an naturally encoded amino acid, e.g., in mammalian cells.

In some embodiments, an amino acid is a compound having the structure of formula A-I:

N(R^{a1})₂-L^{a1}-C(R^{a2})(R^{a3})-L^{a2}-COOH, A-I

or a salt thereof, wherein:
each of Ra, R^{a2}, R^{a3} is independently -L^{a}-R′;
each of L^{a}, L^{a1} and L^{a2} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a compound having the structure of formula A-I or a salt thereof has the structure of NH(R^{a1})-L^{a1}-C(R^{a2})(R^{a3})-L^{a2}-COOH or a salt thereof.

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula A-1 is of the structure NH(R^{a1})—C(R^{a2})(R^{a3})-L^{a2}-COOH.

In some embodiments, L^{a2} is a covalent bond. In some embodiments, a compound of formula A-1 is of the structure NH(R^{a1})—C(R^{a2})(R^{a3})-L^{a2}-COOH.

In some embodiments, L^{a1} is a covalent bond and L^{a2} is a covalent bond. In some embodiments, a compound of formula A-1 is of the structure NH(R^{a1})—C(R^{a2})(R^{a3})—COOH.

In some embodiments, an amino acid is suitable for stapling. In some embodiments, an amino acid comprises a terminal olefin. Certain such amino acids are exemplified herein (e.g., those described in or utilized in peptides of various Tables).

In some embodiments, an agent comprises a detectable moiety, which can either be detected directly or indirectly. For example, in some embodiments, a detectable moiety is or comprises a fluorescent group. In some embodiments, a detectable moiety is or comprises a biotin moiety. In some embodiments, a detectable moiety is connected to the rest of an agent at an amino acid residue, e.g., through a side chain, optionally through a linker (e.g., L as described herein). In some embodiments, a detectable moiety is —N₃, which may be detected after a click chemistry reaction with a labeled agent comprising an alkyne.

In some embodiments, the present disclosure provides various compounds, which among other things may be utilized as amino acids for a number of applications, e.g., for preparation of peptides or other useful compounds.

In some embodiments, a compound (e.g., an amino acid or a protected and/or activated form thereof) or a salt thereof comprises 1) a first group which is an optionally protected amino group, 2) a second group which is an optionally protected and/or activated carboxyl group, and 3) a side chain (typically bonded to an atom between the first and second groups (“a side chain attachment atom”)) which comprises an optionally protected and/or activated carboxyl group and a) an optionally substituted ring (which ring is typically between the optionally protected and/or activated carboxyl group of the side chain and a side chain attachment atom) or b) an amino group (which amino group is typically between the optionally protected and/or activated carboxyl group of the side chain and a side chain attachment atom). In some embodiments, a provided compound is an optionally protected and/or activated amino acid or a salt thereof, wherein the side chain of the amino acid comprises an optionally protected and/or activated carboxyl group, and an optionally substituted ring or an amino group, wherein the optionally substituted ring or an amino group is between the optionally protected and/or activated carboxyl group and a backbone atom to which a side chain is attached (e.g., an atom between an amino and carboxyl group, both of which can be optionally and independently protected and/or activated (e.g., an alpha carbon atom in an amino acid)).

In some embodiments, the present disclosure provides compounds having the structure of formula PA:

N(R^{PA})(R^{a1})-L^{a1}-C(R^{a2})(R^{a3})-L^{a2}-C(O)R^{PC}, PA

or a salt thereof, wherein:
R^{PA} is —H or an amino protecting group;
each of R^{a1} and R^{a3} is independently -L^{a}-R′;
R^{a2} is -L^{aa}-C(O)R^{PS};
each of L^{a}, L^{a1} and L^{a2} is independently L;
—C(O)R^{PS} is optionally protected or activated —COOH;
—C(O)R^{PC} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, compounds (e.g., amino acids, such as those of formula A-I or protected/activated forms thereof) having the structure of formula PA:

N(R^{PA})(R^{a1})-L^{a1}-C(R^{a2})(R^{a3})-L^{a2}-C(O)R^{PC}, PA

or a salt thereof, wherein:
R^{PA} is —H or an amino protecting group;
each of R^{a1} and R^{a3} is independently -L^{a}-R′; R^{a2} is -L^{aa}-C(O)R^{P}s, wherein L^{aa} is L and L^{aa} comprises —N(R′)— or -Cy-;
each of L^{a1} and L^{a2} is independently L;
—C(O)R^{PS} is optionally protected or activated —COOH;
—C(O)R^{PC} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, L^{a1} is a covalent bond. In some embodiments, L^{a1} is not a covalent bond.

In some embodiments, L^{a2} is a covalent bond. In some embodiments, L^{a2} is not a covalent bond.

In some embodiments, R^{a2} is -L^{aa}-C(O)R^{PS}, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with -Cy-.

As used herein, in some embodiments, -Cy- is an optionally substituted bivalent 3-10 (e.g., 3, 4, 5, 6, 7, 8, 9, or 10) membered monocyclic cycloaliphatic group. In some embodiments, -Cy- is an optionally substituted 3-10 (e.g., 3, 4, 5, 6, 7, 8, 9, or 10) membered monocyclic cycloalkyl ring. In some embodiments, -Cy- is an optionally substituted 3-10 (e.g., 3, 4, 5, 6, 7, 8, 9, or 10) membered monocyclic heteroaliphatic ring having 1-5 heteroatoms. In some embodiments, -Cy- is an optionally substituted 3-10 (e.g., 3, 4, 5, 6, 7, 8, 9, or 10) membered monocyclic heteroalkyl ring having 1-5 heteroatoms. In some embodiments, -Cy- is an optionally substituted bivalent 5-15 (e.g., 5, 6, 7, 8, 9, 10, 11, 12, 13, 14, or 15) membered bicyclic or polycyclic cycloaliphatic group. In some embodiments, -Cy- is an optionally substituted bivalent 5-15 (e.g., 5, 6, 7, 8, 9, 10, 11, 12, 13, 14, or 15) membered bicyclic or polycyclic cycloalkyl group. In some embodiments, -Cy- is an optionally substituted 5-15 (e.g., 5, 6, 7, 8, 9, 10, 11, 12, 13, 14, or 15) membered bicyclic or polycyclic heteroaliphatic ring having 1-5 heteroatoms. In some embodiments, -Cy- is an optionally substituted 5-15 (e.g., 5, 6, 7, 8, 9, 10, 11, 12, 13, 14, or 15) membered bicyclic or polycyclic heterocyclyl ring having 1-5 heteroatoms. In some embodiments, a cycloaliphatic, cycloalkyl, heteroaliphatic or heteroalkyl ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is 11-membered. In some embodiments, it is 12-membered. In some embodiments, -Cy- is optionally substituted phenylene. In some embodiments, -Cy- is an optionally substituted bivalent 10-membered bicyclic aryl ring. In some embodiments, -Cy- is an optionally substituted 5-membered heteroaryl ring having 1-4 heteroatoms. In some embodiments, -Cy- is an optionally substituted 6-membered heteroaryl ring having 1-4 heteroatoms. In some embodiments, -Cy- is an optionally substituted 9-membered bicyclic heteroaryl ring having 1-5 heteroatoms. In some embodiments, -Cy- is an optionally substituted 10-membered bicyclic heteroaryl ring having 1-5 heteroatoms. In some embodiments, a heteroaliphatic, heterocyclyl or heteroaryl ring contains no more than 1 heteroatom. In some embodiments, each heteroatom is independently selected from nitrogen, oxygen and sulfur.

In some embodiments, -Cy- is an optionally substituted 4-7 membered ring having 0-3 heteroatoms. In some embodiments, -Cy- is an optionally substituted 6-membered aryl ring. In some embodiments, an aryl ring is substituted. In some embodiments, it is substituted with one or more halogen. In some embodiments, it is substituted with one or more —F. In some embodiments, it is not substituted. In some embodiments, it is optionally substituted

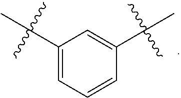 [SMILES: *c1cccc(*)c1]

In some embodiments, it is

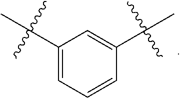 [SMILES: *c1cccc(*)c1]

In some embodiments, it is optionally substituted

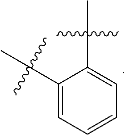 [SMILES: *c1ccccc1*]

In some embodiments, it is

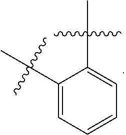 [SMILES: *c1ccccc1*]

In some embodiments, it is optionally substituted

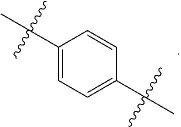 [SMILES: *c1ccc(*)cc1]

In some embodiments, it is

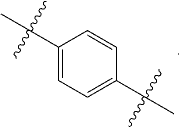 [SMILES: *c1ccc(*)cc1]

In some embodiments, -Cy- is an optionally substituted 5-membered heteroaryl ring having 1-3 heteroatoms. In some embodiments, a heteroatom is nitrogen. In some embodiments, a heteroatom is oxygen. In some embodiments, a heteroatom is sulfur. In some embodiments, -Cy- is optionally substituted

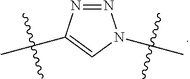 [SMILES: *c1cn(*)nn1]

In some embodiments, -Cy- is

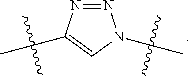 [SMILES: *c1cn(*)nn1]

In some embodiments, L^{aa} is -L^{am1}-Cy-L^{am2}-, wherein each of L^{am1} and L^{am2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L^{aa} comprises -Cy-. In some embodiments, L^{aa} is -L^{am1}-Cy-L^{am2}-, wherein each of L^{am1} and L^{am2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, -L^{an}a- is bonded to —C(O)R^{PS}. In some embodiments, L^{am2} is a covalent bond. In some embodiments, -Cy- is an optionally substituted 4-7 membered ring having 0-3 heteroatoms. In some embodiments, -Cy- is an optionally substituted 5-7 membered ring having 0-3 heteroatoms. In some embodiments, -Cy- is an optionally substituted 6-7 membered ring having 0-3 heteroatoms. In some embodiments, -Cy- is an optionally substituted 4-membered ring having 0-1 heteroatoms. In some embodiments, -Cy- is an optionally substituted 5-membered ring having 0-2 heteroatoms. In some embodiments, -Cy- is an optionally substituted 6-membered ring having 0-2 heteroatoms. In some embodiments, -Cy- is an optionally substituted 7-membered ring having 0-3 heteroatoms.

In some embodiments, R^{a2} is -L^{aa}-C(O)R^{PS}, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with —N(R′)—.

In some embodiments, L^{aa} comprises —N(R′)—. In some embodiments, L^{aa} is -L^{am1}-(NR′)-L^{am2}-, wherein each of L^{am1} and L^{am2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, -L^{an}a- is bonded to —C(O)R^{PS}. In some embodiments, L^{am1} is optionally substituted C₁₋₄ alkylene. In some embodiments, L^{am} is optionally substituted —(CH₂)m-, wherein m is 1, 2, 3, or 4. In some embodiments, L^{am1} is —CH₂—. In some embodiments, L^{am1} is optionally substituted linear C₁₋₂ alkylene. In some embodiments, L^{am1} is —[C(R′)₂]n, wherein n is 1 or 2. In some embodiments, L^{am2} is —[CHR′]n, wherein n is 1 or 2. In some embodiments, each R′ is independently —H or optionally substituted C₁₋₆ alkyl. In some embodiments, L^{am2} is optionally substituted —CH₂—. In some embodiments, L^{am2} is —CH₂—. In some embodiments, R′ is —R^{NR}, wherein R^{NR} is R. In some embodiments, R′ is —CH₂—R^{NR}, wherein R^{NR} is R. In some embodiments, R′ of the —N(R′)— is —C(O)R^{NR}, wherein R^{NR} is R. In some embodiments, R′ of the —N(R′)— is —SO₂R^{NR}, wherein R^{NR} is R. In some embodiments, R is optionally substituted C₁₋₆ aliphatic or heteroaliphatic having 1-4 heteroatoms. In some embodiments, R^{NR} is C₁₋₇ alkyl or heteroalkyl having 1-4 heteroatoms optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms. In some embodiments, R is —CF₃. In some embodiments, L^{am2} is or comprises —C(R′)₂— wherein the R′ group and R′ in —N(R′)— are taken together with their intervening atoms to form an optionally substituted, 3-30 membered, monocyclic, bicyclic or polycyclic ring having, in addition to the intervening atoms, 0-10 heteroatoms.

In some embodiments, L^{aa} is -L^{am1}-N(R′)-L^{am2}-, wherein each of L^{am1} and L^{am2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, —N(R′)— is bonded to two carbon atoms which two carbon atoms do not form any double bonds with heteroatoms. In some embodiments, —N(R′)— is bonded to two sp3 atoms. In some embodiments, —N(R′)— is bonded to two sp3 carbon atoms. In some embodiments, —N(R′)— is bonded to two —CH₂—, each of which is independently and optionally substituted with one or two monovalent substituent. In some embodiments, —N(R′)— is bonded to two —CH₂—.

In some embodiments, L^{aa} comprises —N(R′)—. In some embodiments, R′ of the —N(R′)— is —R^{NR}, wherein R^{NR} is R. In some embodiments, R′ of the —N(R′)— is —CH₂—R^{NR}, wherein R^{NR} is R, and the —CH₂— is optionally substituted. In some embodiments, R′ of the —N(R′)— is —C(O)R^{NR}, wherein R^{NR} is R. In some embodiments, R′ of the —N(R′)— is —SO₂R^{NR}, wherein R^{NR} is R. In some embodiments, —N(R′)— is —N(Et)-. In some embodiments, —N(R′)— is —N(CH₂CF₃)—. In some embodiments, R′ is optionally substituted C₁₋₆ aliphatic or heteroaliphatic having 1-4 heteroatoms. In some embodiments, R′ is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms. In some embodiments, R^{NR} is —CF₃.

In some embodiments, R′ of —N(R′)— is R, R^{a3} is R, and the two R groups are taken together with their intervening atoms to form an optionally substituted 3-10 membered ring having 0-5 heteroatoms in addition to the intervening atoms. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is monocyclic. In some embodiments, a formed ring is bicyclic or polycyclic. In some embodiments, a formed ring is saturated. In some embodiments, a formed ring is partially unsaturated.

In some embodiments, L^{am1} is a covalent bond. In some embodiments, L^{am1} is not a covalent bond. In some embodiments, L^{am1} is optionally substituted C₁₋₄ alkylene. In some embodiments, L^{am1} is optionally substituted —(CH₂)m-, wherein m is 1, 2, 3, or 4. In some embodiments, L^{am1} is optionally substituted —CH₂—. In some embodiments, L^{am1} is —CH₂—.

In some embodiments, L^{am2} is bonded to —C(O)R^{PS}.

In some embodiments, L^{am2} is a covalent bond. In some embodiments, L^{am2} is a covalent bond when it is between -Cy- and —C(O)R^{PS}. In some embodiments, L^{am2} is not a covalent bond. In some embodiments, L^{am2} is optionally substituted C₁₋₄ alkylene. In some embodiments, L^{am2} is optionally substituted —(CH₂)m-, wherein m is 1, 2, 3, or 4. In some embodiments, L^{am2} is optionally substituted linear C₁₋₂ alkylene. In some embodiments, L^{am2} is —[C(R′)₂]n, wherein n is 1 or 2. In some embodiments, L^{am2} is —[CHR′]n, wherein n is 1 or 2. In some embodiments, each R′ is independently —H or optionally substituted C₁₋₆ alkyl. In some embodiments, L^{am2} is optionally substituted —CH₂—. In some embodiments, L^{am2} is —CH₂—. In some embodiments, L^{am2} is optionally substituted —CH₂—CH₂—. In some embodiments, L^{am2} is —CH₂—C(CH₃)₂—.

In some embodiments, L^{am2} is or comprises —C(R′)₂— wherein the R′ group and R′ in —N(R′)— of L^{aa} are taken together with their intervening atoms to form an optionally substituted, 3-30 membered, monocyclic, bicyclic or polycyclic ring having, in addition to the intervening atoms, 0-10 heteroatoms.

In some embodiments, R^{a2} is -L^{aa}-C(O)R^{PS}, wherein L^{aa} is L as described herein. In some embodiments, L^{aa} is L^{am2} as described herein. In some embodiments, L^{aa} is optionally substituted branched or linear C₁₋₁₀ hydrocarbon chain. In some embodiments, L^{aa} is optionally substituted C₁₋₁₀ (e.g., 1, 2, 3, 4, 5, 6, 7, 8, 9, or 10) alkylene. In some embodiments, L^{aa} is optionally substituted —CH₂—CH₂—. In some embodiments, L^{aa} is —CH₂—CH₂—. In some embodiments, L^{aa} is optionally substituted —CH₂—. In some embodiments, L^{aa} is —CH₂—.

In some embodiments, L^{a} is L^{aa} as described herein.

In some embodiments, L^{aa} is L^{a} as described herein.

As described above, each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L is a covalent bond.

In some embodiments, L (or L^{a}, L^{aa}, L^{a1}, L^{a2}, L^{s1}, L^{s2}, L^{s3}, or another variable or moiety that can be L, or a linker moiety) is an optionally substituted, bivalent C₁-C₂₅, C₁-C₂₀, C₁-C₁₅, C₁-C₁₀, C₁-C₉, C₁-C₈, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₂₅, C₁-C₂₀, C₁-C₁₅, C₁-C₁₀, C₁-C₉, C₁-C₈, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₁-C₁₀, C₁-C₉, C₁-C₅, C₁-C₇, C₁-C₆, C₁-C₅, C₁-C₄, C₁-C₃, C₁-C₂, or C₁, C₂, C₃, C₄, C₅, C₆, C₇, C₈, C₉, or C₁₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₂ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₃ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₄ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₅ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the bivalent aliphatic is saturated. In some embodiments, the bivalent aliphatic is linear. In some embodiments, the bivalent aliphatic is branched. In some embodiments, it is an optionally substituted, bivalent linear saturated C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with —O—, —S—, —N(R′)—, —C(O)—, —S(O)—, —S(O)₂—, —S(O)₂N(R′)—, —C(O)S—, or —C(O)O—. In some embodiments, each replacement if any is independently with —O—, —S—, —N(R′)—, or —C(O)—. In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₆ linear saturated aliphatic wherein one or more methylene units is optionally and independently replaced with —O—, —S—, —N(R′)—, or —C(O)—. In some embodiments, it is an optionally substituted, bivalent C₁-C₅ linear saturated aliphatic wherein one or more methylene units is optionally and independently replaced with —O—, —S—, —N(R′)—, or —C(O)—. In some embodiments, it is an optionally substituted, bivalent C₁-C₄ linear saturated aliphatic wherein one or more methylene units is optionally and independently replaced with —O—, —S—, —N(R′)—, or —C(O)—. In some embodiments, it is an optionally substituted, bivalent C₁-C₃ linear saturated aliphatic wherein one or more methylene units is optionally and independently replaced with —O—, —S—, —N(R′)—, or —C(O)—. In some embodiments, it is an optionally substituted, bivalent C₁-C₂ linear saturated aliphatic wherein one or more methylene units is optionally and independently replaced with —O—, —S—, —N(R′)—, or —C(O)—. In some embodiments, it is a bivalent C₁-C₆ linear saturated aliphatic wherein one or more methylene units is optionally and independently replaced with —O—, —S—, —N(R′)—, or —C(O)—. In some embodiments, it is a bivalent C₁-C₅ linear saturated aliphatic wherein one or more methylene units is optionally and independently replaced with —O—, —S—, —N(R′)—, or —C(O)—. In some embodiments, it is a bivalent C₁-C₄ linear saturated aliphatic wherein one or more methylene units is optionally and independently replaced with —O—, —S—, —N(R′)—, or —C(O)—. In some embodiments, it is a bivalent C₁-C₃ linear saturated aliphatic wherein one or more methylene units is optionally and independently replaced with —O—, —S—, —N(R′)—, or —C(O)—. In some embodiments, it is a bivalent C₁-C₂ linear saturated aliphatic wherein one or more methylene units is optionally and independently replaced with —O—, —S—, —N(R′)—, or —C(O)—. In some embodiments, there is no replacement of methylene unit. In some embodiments, there is one replacement. In some embodiments, there is two replacement. In some embodiments, there is three replacement. In some embodiments, there is four or more replacement. In some embodiments, R′ in each moiety that is utilized to replace a methylene unit (e.g., —N(R′)—) as described herein is hydrogen or optionally substituted C₁₋₆ aliphatic or phenyl. In some embodiments, R′ is each such moiety is hydrogen or optionally substituted C₁₋₆ alkyl. In some embodiments, R′ is each such moiety is hydrogen or C₁₋₆ alkyl. In some embodiments, each -Cy- is optionally substituted bivalent ring selected from 3-10, 3-9, 3-8, 3-7, 5-10, 5-9, 5-8, 5-7, 5-6, or 3, 4, 5, 6, 7, 8, 9, or 10 membered cycloaliphatic and heterocyclylene having 1-3 heteroatoms, phenylene, and 5-6 membered heteroarylene having 1-3 heteroatoms. In some embodiments, -Cy- is optionally substituted bivalent 3-10, 3-9, 3-8, 3-7, 5-10, 5-9, 5-8, 5-7, 5-6, or 3, 4, 5, 6, 7, 8, 9, or 10 membered cycloaliphatic. In some embodiments, -Cy- is optionally substituted 3-10, 3-9, 3-8, 3-7, 5-10, 5-9, 5-8, 5-7, 5-6, or 3, 4, 5, 6, 7, 8, 9, or 10 membered heterocyclylene having 1-3 heteroatoms. In some embodiments, -Cy- is optionally substituted 3-10, 3-9, 3-8, 3-7, 5-10, 5-9, 5-8, 5-7, 5-6, or 3, 4, 5, 6, 7, 8, 9, or 10 membered heterocyclylene having 1 heteroatom. In some embodiments, -Cy- is optionally substituted phenylene. In some embodiments, -Cy- is phenylene. In some embodiments, -Cy- is optionally substituted 5-6 membered heteroarylene having 1-3 heteroatoms. In some embodiments, -Cy- is optionally substituted 5-6 membered heteroarylene having 1 heteroatom. In some embodiments, a heteroatom is nitrogen. In some embodiments, a heteroatom is oxygen. In some embodiments, a heteroatom is sulfur. In some embodiments, L, L^{a}, L^{aa}, L^{a1}, L^{a2}, L^{s1}, L^{s2}, L^{s3}, L″, or another variable or moiety that can be L, or a linker moiety, is optionally substituted —(CH₂)n-. In some embodiments, it is —(CH₂)n-. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, L, L^{a}, L^{aa} L^{a1}, L^{a2}, L^{s1}, L^{s2}, L^{s3}, L″, or another variable or moiety that can be L, or a linker moiety, is an optionally substituted, bivalent heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

Those skilled in the art appreciate that embodiments described for one linker moiety that can be L or L″ (e.g., L^{aa}, L^{s1}, L^{s2}, L^{s3}, L^{s}, L^{a}, L^{a1}, L^{a2}, L^{RN}, etc.) may also be utilized for another group that can be L or L″ to the extent that such embodiments fall within the definition of L or L″.

As described above, each R′ is independently —R, —C(O)R, —CO₂R, or —SO₂R. In some embodiments, R′ is -L^{a}-R. In some embodiments, R′ is R. In some embodiments, R′ is —C(O)R. In some embodiments, R′ is —CO₂R. In some embodiments, R′ is —SO₂R. In some embodiments, R′ is —H.

As described above, each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

As described herein, in some embodiments, R is —H. In some embodiments, R is not —H. In some embodiments, R is optionally substituted C₁₋₁₀ aliphatic. In some embodiments, R is optionally substituted C₁₋₁₀ alkyl. In some embodiments, R is methyl. In some embodiments, R is ethyl. In some embodiments, R is isopropyl. In some embodiments, R is —CF₃. In some embodiments, R is —CH₂CF₃. In some embodiments, R is butyl. In some embodiments, R is t-butyl. In some embodiments, R is optionally substituted C₃₋₁₀ cycloaliphatic. In some embodiments, R is optionally substituted C₃₋₁₀ cycloalkyl. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted phenyl. In some embodiments, R is phenyl. In some embodiments, R is optionally substituted 5-membered heteroaryl having 1-3 heteroatoms. In some embodiments, R is optionally substituted 5-membered heteroaryl having 1 heteroatom. In some embodiments, R is optionally substituted 6-membered heteroaryl having 1-3 heteroatoms. In some embodiments, R is optionally substituted 6-membered heteroaryl having 1 heteroatom. In some embodiments, R is optionally substituted bicyclic 8-10 membered aromatic ring having 0-5 heteroatoms. In some embodiments, R is optionally substituted bicyclic 9-membered aromatic ring having 1-5 heteroatoms. In some embodiments, R is optionally substituted bicyclic 10-membered aromatic ring having 1-5 heteroatoms. In some embodiments, R is optionally substituted bicyclic 9-membered aromatic ring having 1 heteroatom. In some embodiments, R is optionally substituted bicyclic 10-membered aromatic ring having 1 heteroatom. In some embodiments, R is optionally substituted bicyclic 10-membered aromatic ring having no heteroatom. In some embodiments, R is optionally substituted 3-10 membered heterocyclyl having 1-5 heteroatoms. In some embodiments, R is optionally substituted 5-14 membered bicyclic heterocyclyl having 1-5 heteroatoms.

In some embodiments, two R groups (or two groups that can be R, e.g., two groups each independently selected from R′, R^{a1}, R^{a2}, R^{a3}, R^{a5}, R^{RN}, etc.) are taken together with their intervening atom(s) to form an optionally substituted 3-30 membered, monocyclic, bicyclic or polycyclic ring having, in addition to the atom, 0-10 heteroatoms. In some embodiments, a formed ring is substituted. In some embodiments, a formed ring is unsubstituted. In some embodiments, a formed ring is 3-30, 3-20, 3-15, 3-10, 3-9, 3-8, 3-7, 3-6, 4-10, 4-9, 4-8, 4-7, 4-6, 5-10, 5-9, 5-8, 5-7, 5-6, or 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 membered. In some embodiments, a formed ring is 3-10 membered. In some embodiments, a formed ring is 3-7 membered. In some embodiments, a formed ring is 4-10 membered. In some embodiments, a formed ring is 4-7 membered. In some embodiments, a formed ring is 5-10 membered. In some embodiments, a formed ring is 5-7 membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is monocyclic. In some embodiments, a formed ring is bicyclic. In some embodiments, a formed ring is polycyclic. In some embodiments, a formed ring has no heteroatoms in addition to the intervening atom(s). In some embodiments, a formed ring has 1-10, e.g., 1-5, 1-3, or 1, 2, 3, 4, 5, 6, 7, 8, 9, or 10 heteroatoms in addition to the intervening atom(s). In some embodiments, a formed ring is saturated. In some embodiments, a formed ring is partially unsaturated. In some embodiments, a formed ring comprises one or more aromatic ring. In some embodiments, a formed ring is bicyclic or polycyclic, and each monocyclic unit is independently 3-10 membered, saturated, partially unsaturated or aromatic and having 0-5 heteroatoms. In some embodiments, each heteroatom is independently selected from nitrogen, oxygen and sulfur.

In some embodiments, a group that can be R, e.g., R′, R^{a1}, R^{a2}, R^{a3}, R^{a5}, R^{RN}, etc., is R as described herein. Those skilled in the art appreciate that embodiments described for one group that can be R may also be utilized for another group that can be R to the extent that such embodiments fall within the definition of R.

In some embodiments, the present disclosure provides compounds having the structure of

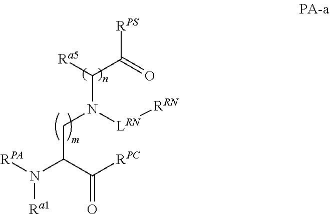

or a salt thereof, wherein:
each of m and n is independently 1, 2, 3, or 4;
L^{RN} is L;
R^{RN} is R;
R^{a5} is R′; and
each other variable is independently as described herein.

In some embodiments, m is 1. In some embodiments, m is 2. In some embodiments, m is 3. In some embodiments, m is 4.

In some embodiments, n is 1. In some embodiments, n is 2. In some embodiments, n is 3. In some embodiments, n is 4.

In some embodiments, L^{RN} is —CH₂—, —CO—, or —SO₂—. In some embodiments, L^{RN} is —CH₂—. In some embodiments, L^{RN} is —CO—. In some embodiments, L^{RN} is —SO₂—. In some embodiments, L^{RN} is optionally substituted bivalent C₁₋₄ alkylene. In some embodiments, L^{RN} is optionally substituted bivalent linear C₁₋₄ alkylene. In some embodiments, L^{RN} is —CH₂—CH₂—. In some embodiments, L^{RN} is —CH₂—CH₂—CH₂—. In some embodiments, L^{RN} is —C(CH₃)—.

In some embodiments, R^{RN} is R as described herein. In some embodiments, R^{RN} is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.

In some embodiments, R (e.g., R^{RN}, R′, etc.) is optionally substituted aliphatic, e.g., C₁₋₁₀ aliphatic. In some embodiments, R is optionally substituted alkyl, e.g., C₁₋₁₀ alkyl. In some embodiments, R is optionally substituted cycloalkyl, e.g., C₁₋₁₀ cycloalkyl. In some embodiments, R is optionally substituted aryl. In some embodiments, R is optionally substituted heterocyclyl. In some embodiments, R is optionally substituted heteroaryl. In some embodiments, is methyl. In some embodiments, R is —CF₃. In some embodiments, R is ethyl. In some embodiments, R is

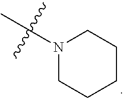 [SMILES: *N1CCCCC1]

In some embodiments, R is phenyl. In some embodiments, R is pentafluorophenyl. In some embodiments, R is pyridinyl.

In some embodiments, one or more R^{a5} are independently —H. In some embodiments, one or more R^{a5} are independently optionally substituted C₁₋₆ alkyl. In some embodiments, each R^{a5} is —H.

In some embodiments, -L^{RN}-R^{RN} is R, and is taken together with a R^{a5} and their intervening atoms to form an optionally substituted, 3-30 membered, monocyclic, bicyclic or polycyclic ring having, in addition to the intervening atoms, 0-10 heteroatoms.

As described in the present disclosure, various rings, including those in various moieties (e.g., R or various groups that can be R, various bivalent rings such as those in -Cy-) and those formed by two entities (e.g., two groups that are or can be R) taken together with their intervening forms, can be various sizes, e.g., 3-30. In some embodiments, a ring is 3-30-membered. In some embodiments, a ring is 3-20 membered. In some embodiments, a ring is 3-10 membered. In some embodiments, a ring is e.g., 3, 4, 5, 6, 7, 8, 9, or 10-membered. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. In some embodiments, a ring is substituted (in addition to potential groups already drawn out in formulae). In some embodiments, a ring is not substituted. In some embodiments, a ring is saturated. In some embodiments, a ring is partially unsaturated. In some embodiments, a ring is aromatic. In some embodiments, a ring comprise one or more, e.g., 1-5, heteroatoms. In some embodiments, one or more heteroatoms are oxygen. In some embodiments, one or more heteroatoms are nitrogen. In some embodiments, one or more heteroatoms are sulfur. In some embodiments, a ring is a cycloaliphatic, e.g., cycloalkyl ring. In some embodiments, a ring is a heterocycloaliphatic, e.g., heterocycloalkyl ring. In some embodiments, a ring is an aryl ring. In some embodiments, a ring is a heteroaryl ring. In some embodiments, a ring is a heteroaryl ring. In some embodiments, a ring is monocyclic. In some embodiments, a ring is bicyclic or polycyclic. In some embodiments, each monocyclic unit in a ring is independently an optionally substituted, 3-10 membered (e.g., 3, 4, 5, 6, 7, 8, 9, or 10-membered), saturated, partially unsaturated or aromatic ring having 0-5 heteroatoms.

As described herein, in some embodiments, a heteroatom is selected from nitrogen, oxygen, sulfur, silicon and phosphorus. As described herein, in some embodiments, a heteroatom is selected from nitrogen, oxygen, and sulfur.

In some embodiments, R^{a1} is —H. In some embodiments, R^{a1} is optionally substituted C₁₋₆ alkyl. In some embodiments, R^{a1} are taken together with another group, e.g., R^{a3} and their intervening atoms to form an optionally substituted ring as described herein.

In some embodiments, —C(O)R^{PC} is a protected carboxylic acid group. In some embodiments, —C(O)R^{PC} is an activated carboxylic acid group. Those skilled in the art will appreciate that various groups are available for protecting/activating carboxyl groups, including various groups that are useful in peptide synthesis, and can be utilized in accordance with the present disclosure. In some embodiments, —C(O)R^{PC} is an ester. In some embodiments, —C(O)R^{PC} is an activated ester for synthesis. In some embodiments, —C(O)R^{PC} is —C(O)OR′. In some embodiments, R′ is R. In some embodiments, R′ is optionally substituted C₁₋₁₀ aliphatic. In some embodiments, R′ optionally substituted phenyl. In some embodiments, R′ is pentafluorophenyl. In some embodiments, R′ is

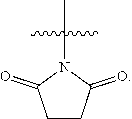 [SMILES: *N1C(=O)CCC1=O]

In some embodiments, —C(O)R^{PC} is —COOH.

In some embodiments, —C(O)R^{PS} is a protected carboxylic acid group. In some embodiments, —C(O)R^{PS} is an activated carboxylic acid group if it is to be reacted with another moiety. Those skilled in the art will appreciate that various groups are available for protecting/activating carboxyl groups, including various groups that are useful in peptide synthesis, and can be utilized in accordance with the present disclosure. In some embodiments, —C(O)R^{PS} is an ester. In some embodiments, —C(O)R^{PS} is an ester. In some embodiments, —C(O)R^{PS} is —C(O)OR′. In some embodiments, R′ is R. In some embodiments, R is optionally substituted C₁₋₁₀ aliphatic. In some embodiments, R optionally substituted phenyl. In some embodiments, R is optionally substituted t-Bu. In some embodiments, R is t-Bu. In some embodiments, R is benzyl. In some embodiments, R is allyl. In some embodiments, —C(O)R^{PS} is a protected carboxylic acid group that is compatible with peptide synthesis (e.g., Fmoc-based peptide synthesis). In some embodiments, —C(O)R^{PS} is a protected carboxylic acid group which is orthogonal to —C(O)R^{PC} and R^{PA}, and remains intact when —C(O)R^{PC} and/or N(R^{PA})(R^{a1}) are protected, deprotected, and/or reacted (e.g., in peptide synthesis such as Fmoc-based peptide synthesis). In some embodiments, —C(O)R^{PS} is deprotected at a late stage during synthesis, e.g., after a peptide backbone is or is largely constructed such that an unprotected side chain —COOH does not impact synthesis.

In some embodiments, —C(O)R^{PS} is —COOH.

As described above, R^{PA} is —H or an amino protecting group. In some embodiments, R^{PA} is —H. In some embodiments, R^{PA} is an amino protecting group. In some embodiments, R^{PA} is an amino protecting group suitable for peptide synthesis. In some embodiments, R^{PA} is —C(O)—O—R, wherein R is optionally substituted

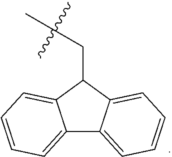 [SMILES: *CC1c2ccccc2-c2ccccc21]

In some embodiments, R^{PA} is —Fmoc. In some embodiments, R^{PA} is —Cbz. In some embodiments, R^{AA} is -Boc.

In some embodiments, R^{PS} is a protecting group orthogonal to R^{PA}. In some embodiments, R^{PS} is a protecting group orthogonal to R^{PC}. In some embodiments, R^{PS} is compatible with peptide synthesis. In some embodiments, R^{PS} is optionally substituted C₁₋₆ aliphatic. In some embodiments, R^{PS} is t-butyl.

In some embodiments, R^{PS} is —S-L-R′, wherein each variable is independently as described herein. In some embodiments, L is optionally substituted —CH₂—. In some embodiments, L is —CH₂—. In some embodiments, R^{PS} is —S—CH₂—R′, wherein R′ is as described herein. In some embodiments, R′ is R as described herein. In some embodiments, R is optionally substituted C₆-30 aryl. In some embodiments, R is optionally substituted C₆₋₁₀ aryl. In some embodiments, R is optionally substituted phenyl. In some embodiments, R is phenyl. In some embodiments, R is substituted phenyl wherein one or more substituents are independently alkoxy. In some embodiments, R is 2, 4, 6-trimethoxyphenyl. In some embodiments, R is optionally substituted 5-30 membered heteroaryl having 1-10 heteroatoms. In some embodiments, R is optionally substituted 5-10 membered heteroaryl having 1-4 heteroatoms. In some embodiments, R is optionally substituted 5-membered heteroaryl having 1-4 heteroatoms. In some embodiments, R^{PS} is —S—CH₂-Cy-R′, wherein the —CH₂— is optionally substituted, and -Cy- is as described herein. In some embodiments, R^{PS} is —S—CH₂-Cy-O—R′, wherein the —CH₂— is optionally substituted, and -Cy- is as described herein. In some embodiments, -Cy- is an optionally substituted aromatic ring. In some embodiments, -Cy- is optionally substituted phenylene. In some embodiments, -Cy- is 2, 6-dimethoxy-1, 4-phenylene. In some embodiments, -Cy- is 2, 4, 6-trimethoxy-1, 3-phenylene. In some embodiments, R^{PS} is

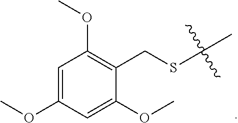 [SMILES: *SCc1c(OC)cc(OC)cc1OC]

In some embodiments, R^{PS} is —SH.

In some embodiments, R^{a2} is

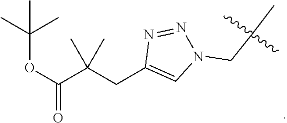 [SMILES: *Cn1cc(CC(C)(C)C(=O)OC(C)(C)C)nn1]

In some embodiments, R^{a2} is

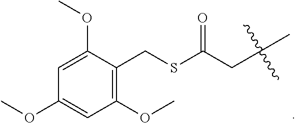 [SMILES: *CC(=O)SCc1c(OC)cc(OC)cc1OC]

In some embodiments, R^{a2} is

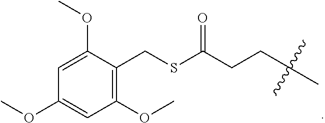 [SMILES: *CCC(=O)SCc1c(OC)cc(OC)cc1OC]

In some embodiments, R² is

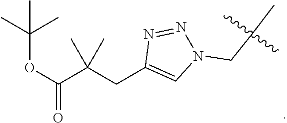 [SMILES: *Cn1cc(CC(C)(C)C(=O)OC(C)(C)C)nn1]

In some embodiments, —C(R^{a2})(R^{a3})— is

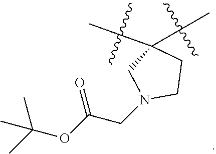

In some embodiments, a provided compound, e.g., an amino acid, is selected from:

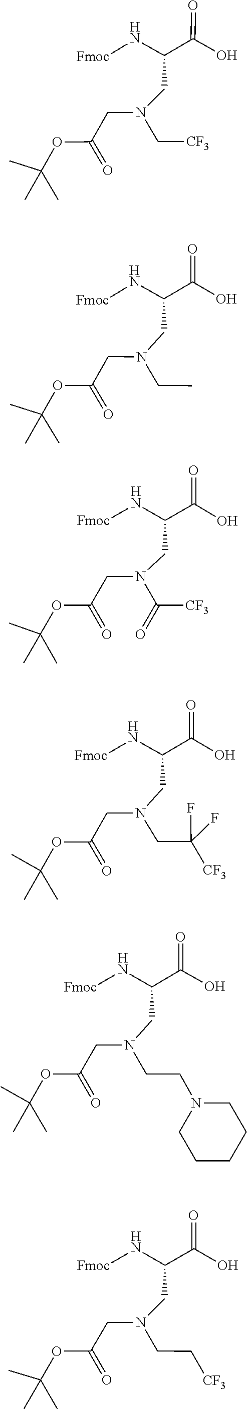

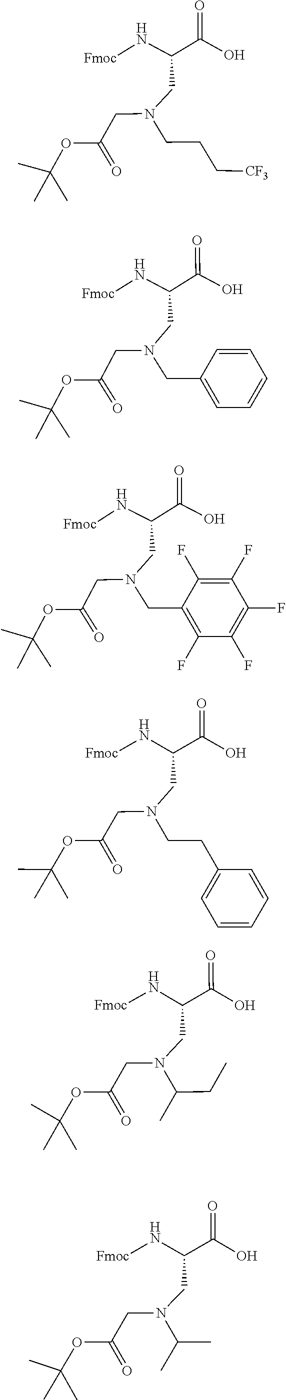

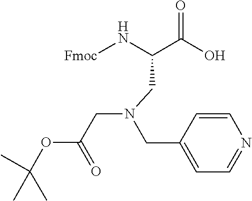

In some embodiments, R^{a2} is R^{a2} in a compound described above (a non-hydrogen group attached to an alpha carbon).

In some embodiments, the present disclosure provides compounds having the structure of:

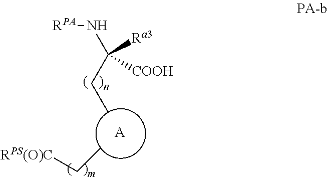

or a salt thereof, wherein:
Ring A is an optionally substituted 3-10 membered ring;
n is 0-6;
m is 0-6; and
each other variable is independently as described herein.

In some embodiments, m is 0. In some embodiments, m is 1-6.

In some embodiments, the present disclosure provides compounds having the structure of:

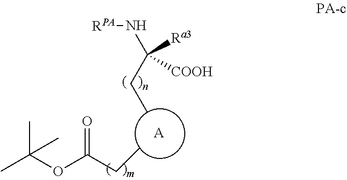

or a salt thereof, wherein:
Ring A is an optionally substituted 3-10 membered ring;
n is 0-6;
m is 0-6; and
each other variable is as described herein.

In some embodiments, m is 0. In some embodiments, m is 1-6.

In some embodiments, the present disclosure provides compounds having the structure of:

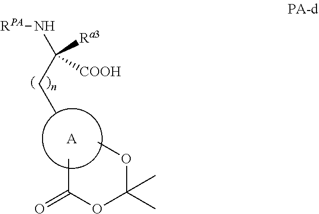

or a salt thereof, wherein:
Ring A is an optionally substituted 3-10 membered ring;
n is 0-6; and
each other variable is as described herein.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 0, 1, or 2.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 1, 2, or 3.

In some embodiments, Ring A is a ring as described herein. In some embodiments, Ring A is 3-membered. In some embodiments, Ring A is 4-membered. In some embodiments, Ring A is 5-membered. In some embodiments, Ring A is 6-membered. In some embodiments, Ring A is 7-membered. In some embodiments, Ring A is 8-membered. In some embodiments, Ring A is 9-membered. In some embodiments, Ring A is 10-membered. In some embodiments, Ring A is saturated. In some embodiments, Ring A is partially unsaturated. In some embodiments, Ring A is aromatic. In some embodiments, Ring A has no additional heteroatoms in addition to the nitrogen atom. In some embodiments, Ring is unsubstituted. In some embodiments, Ring A is substituted with one or more halogen. In some embodiments, Ring A is substituted with one or more —F. In some embodiments, Ring A has a carbon substituted with two —F. In some embodiments, —C(O)R^{PS} is at 2′-position (N being position 1). In some embodiments, —C(O)R^{PS} is at 3′-position. In some embodiments, —C(O)R^{PS} is at 4′-position. In some embodiments, —C(O)R^{PS} is attached to a chiral center, e.g., a chiral carbon atom. In some embodiments, a chiral center is R. In some embodiments, a chiral center is S. In some embodiments, Ring A is bonded to —(CH₂)n- at a chiral carbon which is R. In some embodiments, Ring A is bonded to —(CH₂)n- at a chiral carbon which is S. In some embodiments, —(CH₂)n- is at position 2 (the N is at position 1). In some embodiments, —(CH₂)n- is at position 3 (the N is at position 1). In some embodiments, —(CH₂)n- is at position 4 (the N is at position 1).

In some embodiments, Ring A is substituted. In some embodiments, substituents on Ring A are of suitable properties, e.g., volumes, for various utilizations. In some embodiments, substituents are independently selected from halogen, —R, —CF₃, —N(R)₂, —CN, and —OR, wherein each R is independently C₁₋₆ aliphatic optionally substituted with one or more —F. In some embodiments, substituents are independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN. In some embodiments, substituents are selected from halogen, a C₅₋₆ aromatic ring having 0-4 heteroatoms, and an optionally substituted 3-10 membered cycloalkyl or heteroalkyl ring having 1-4 heteroatoms. In some embodiments, a substituent is halogen. In some embodiments, it is —F. In some embodiments, it is —Cl. In some embodiments, it is —Br. In some embodiments, it is —I. In some embodiments, a substituent is optionally substituted C₁₋₄ alkyl. In some embodiments, a substituent is C₁₋₄ alkyl. In some embodiments, it is methyl. In some embodiments, it is ethyl. In some embodiments, it is i-Pr. In some embodiments, a substituent is C₁₋₄ haloalkyl. In some embodiments, a substituent is C₁₋₄ alkyl optionally substituted with one or more —F. In some embodiments, it is —CF₃. In some embodiments, it is —CN. In some embodiments, it is —OR wherein R is optionally substituted C₁₋₄ alkyl. In some embodiments, it is —OR wherein R is C₁₋₄ alkyl. In some embodiments, it is —OR wherein R is C₁₋₄ haloalkyl. In some embodiments, it is —OR wherein R is C₁₋₄ alkyl optionally substituted with one or more —F. In some embodiments, it is —OCF₃.

In some embodiments, Ring A is or comprises an optionally substituted saturated monocyclic ring. In some embodiments, Ring A is or comprises an optionally substituted partially unsaturated monocyclic ring. In some embodiments, Ring A is or comprises an optionally substituted aromatic monocyclic ring. In some embodiments, Ring A is optionally substituted phenyl. In some embodiments, Ring A is optionally substituted 5-6 membered heteroaryl having 1-3 heteroatoms. In some embodiments, Ring A is optionally substituted 5-6 membered heteroaryl having 1-3 heteroatoms, wherein at least one heteroatom is nitrogen. In some embodiments, Ring A is an optionally substituted 8-10 membered bicyclic ring having 1-6 heteroatoms. In some embodiments, Ring A is an optionally substituted 8-10 membered bicyclic aromatic ring having 1-6 heteroatoms, wherein each monocyclic unit is independently an optionally 5-6 membered aromatic ring having 0-3 heteroatoms. In some embodiments, Ring A is bonded to —(CH₂)n- at a carbon atom. In some embodiments, Ring A is bonded to —(CH₂)n- at a nitrogen atom. In some embodiments, Ring A or -Cy- in L^{aa} is optionally substituted, and each substitute is independently selected from halogen, —R, —CF₃, —N(R)₂, —CN, and —OR, wherein each R is independently C₁₋₆ aliphatic optionally substituted with one or more —F. In some embodiments, Ring A or -Cy- in L^{aa} is optionally substituted, and each substitute is independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN.

In some embodiments, Ring A is optionally substituted phenyl. In some embodiments, the present disclosure provides a compound of formula

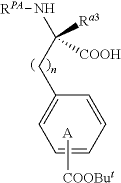

or a salt thereof, wherein Ring A is optionally substituted phenyl, and each variable is as described herein.

In some embodiments, the present disclosure provides compounds having the structure of

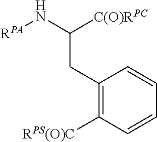

or a salt thereof, wherein each variable is independent as described herein. In some embodiments, the present disclosure provides compounds having the structure of

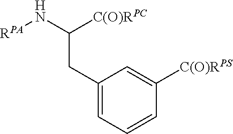

or a salt thereof, wherein each variable is independent as described herein.

In some embodiments, a compound is selected from:

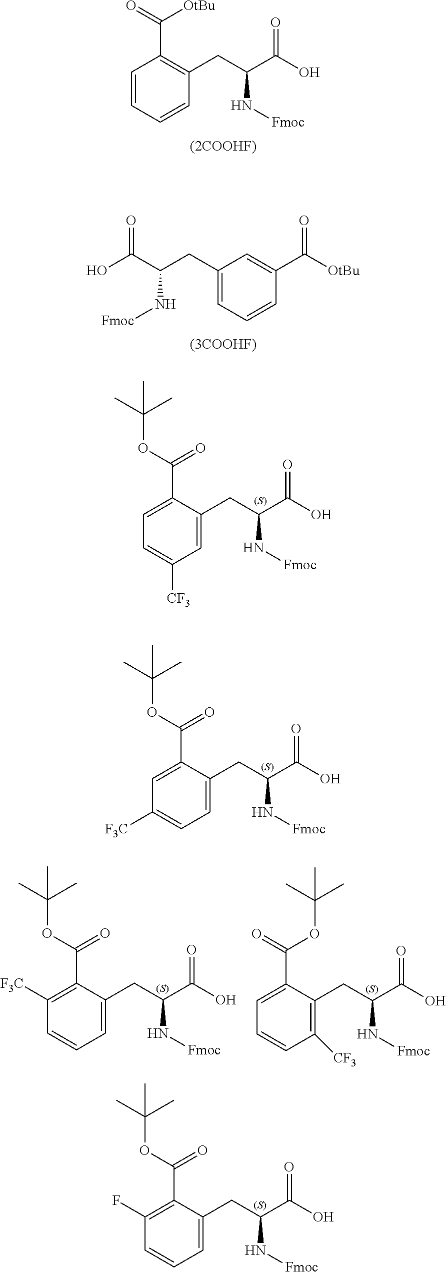

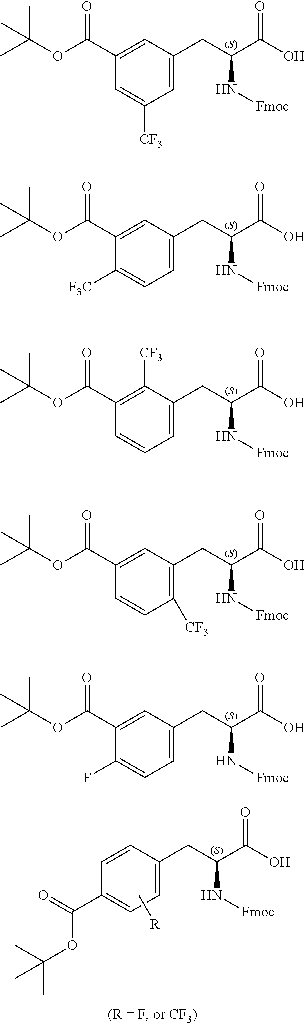

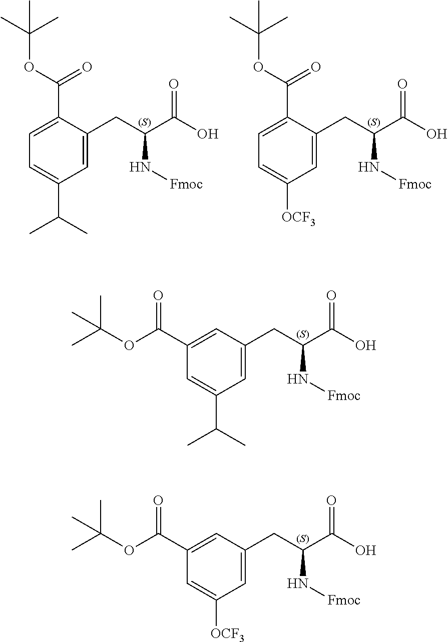

In some embodiments, the present disclosure provides a compound of formula

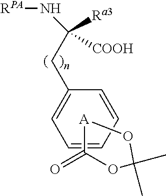

or a salt thereof, wherein Ring A is optionally substituted phenyl, and each variable is as described herein. In some embodiments, a compound is selected from:

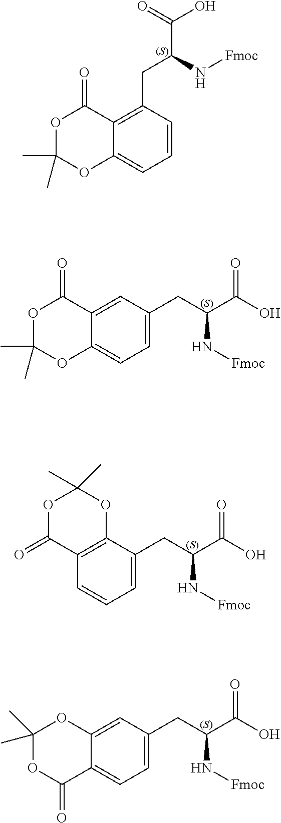

In some embodiments, Ring A is an optionally substituted 5- or 6-membered heteroaryl having 1-4 heteroatoms. In some embodiments, a provided compound has the structure of

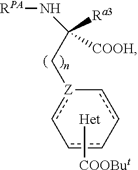

wherein Z is carbon or a heteroatom, Ring Het is an optionally substituted 5- or 6-membered heteroaryl having 1-4 heteroatoms, and each other variable is independently as described herein. In some embodiments, a provided compound is selected from:

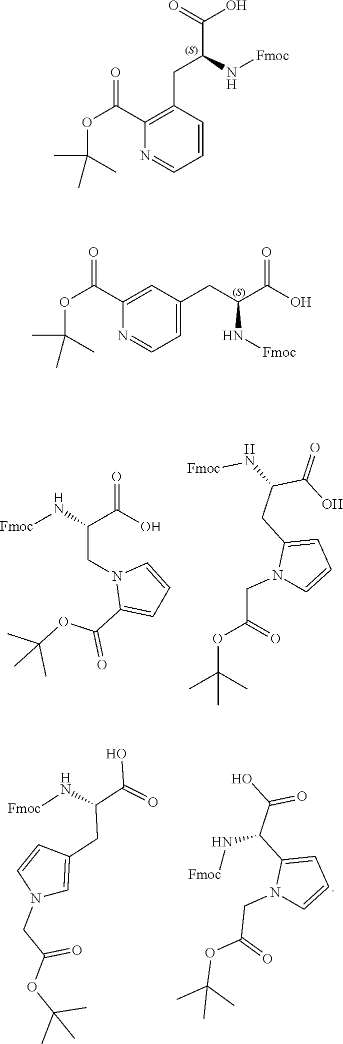

In some embodiments, Ring A is a 8-10 membered bicyclic aryl or a heteroaryl ring having 1-5 heteroatoms. In some embodiments, Ring A is a 10-membered bicyclic aryl ring. In some embodiments, Ring A is a 8-membered bicyclic heteroaryl ring having 1-5 heteroatoms. In some embodiments, Ring A is a 9-membered bicyclic heteroaryl ring having 1-5 heteroatoms. In some embodiments, Ring A is a 10-membered bicyclic heteroaryl ring having 1-5 heteroatoms. In some embodiments, Ring A is an optionally substituted 5- or 6-membered heteroaryl having 1-4 heteroatoms. In some embodiments, a provided compound has the structure of

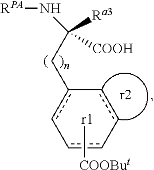

wherein each of Ring r1 and r2 is independently an optionally substituted 5- or 6-membered aryl or heteroaryl ring having 1-4 heteroatoms, and each other variable is independently as described herein. In some embodiments, a provided compound has the structure of

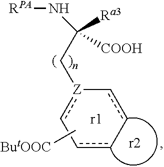

wherein Z is carbon or a heteroatom, each of Ring r1 and r2 is independently an optionally substituted 5- or 6-membered aryl or heteroaryl ring having 1-4 heteroatoms, and each other variable is independently as described herein. In some embodiments, a provided compound is selected from:

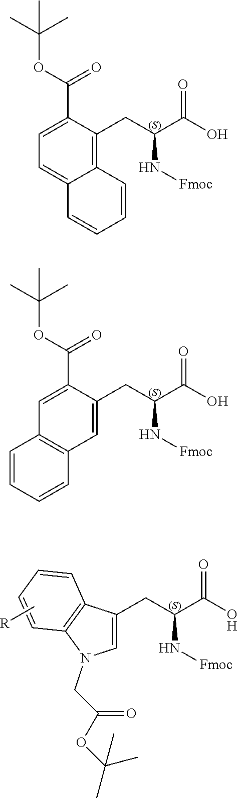

In some embodiments, the present disclosure provides a compound of structure

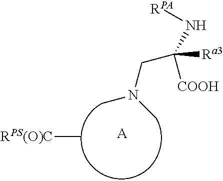

or a salt thereof. In some embodiments, —C(O)R^{PS} is —C(O)—OtBu. In some embodiments, the present disclosure provides a compound of structure

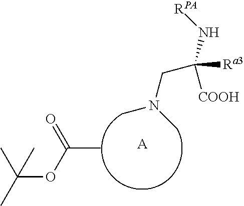

or a salt thereof, wherein each variable is independently as described herein.

In some embodiments, a provided compound is selected from:

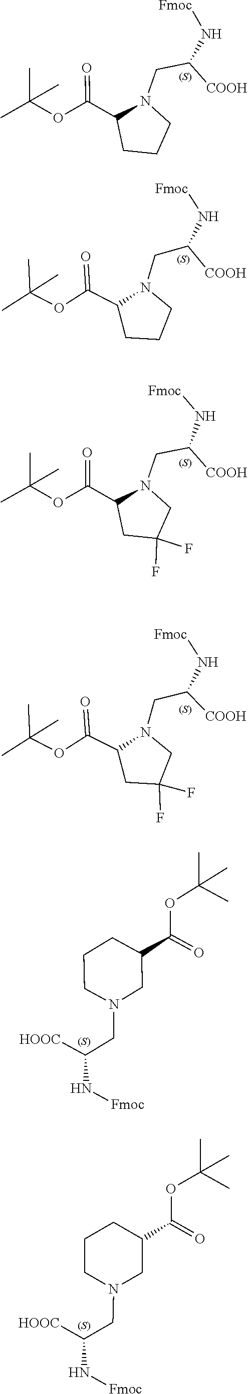

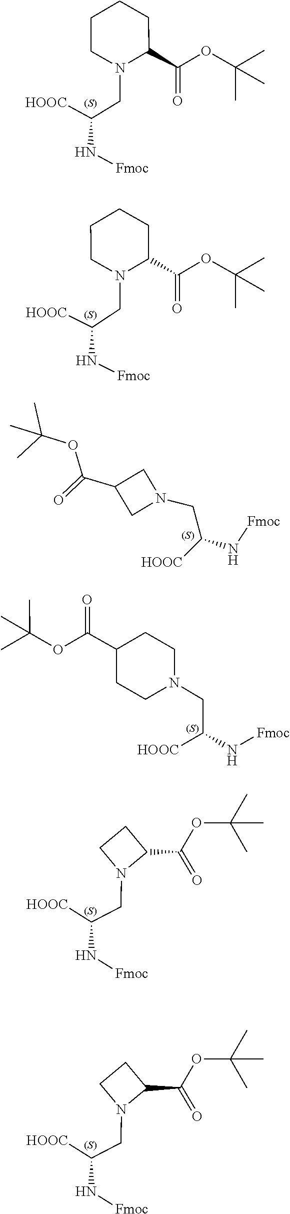

In some embodiments, the present disclosure provides compounds having the structure of

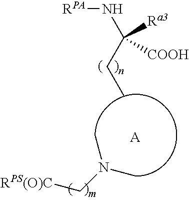

or a salt thereof, wherein each variable is independently as described herein. In some embodiments, the present disclosure provides compounds having the structure of

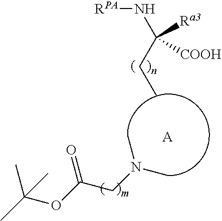

or a salt thereof, wherein each variable is independently as described herein.

In some embodiments, a provided compound is selected from:

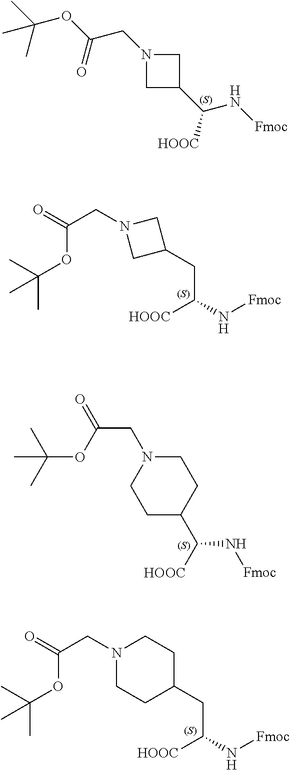

In some embodiments, a provided compound is an amino acid. In some embodiments, a provided compound is a protected amino acid. In some embodiments, a provided compound is a protected and/or activated amino acid. In some embodiments, a provided compound is suitable for

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula PA is of the structure NH(R^{a1})—C(R^{a2})(R^{a3})-L^{a2}-COOH.

In some embodiments, L^{a2} is a covalent bond. In some embodiments, a compound of formula PA is of the structure NH(R^{a1})—C(R^{a2})(R^{a3})-L^{a2}-COOH.

In some embodiments, Lai is a covalent bond and L^{a2} is a covalent bond. In some embodiments, a compound of formula PA is of the structure NH(R^{a1})—C(R^{a2})(R^{a3})—COOH.

In some embodiments, an amino acid is suitable for stapling. In some embodiments, an amino acid comprises a terminal olefin.

In some embodiments, an amino acid has the structure of NH(R^{a1})-L^{a1}-C(-L^{aa}-COOH)(R^{a3})-L^{a2}-COOH, or a salt thereof, wherein each variable is independently as described in the present disclosure. In some embodiments, L^{aa} is -L^{am1}-N(R′)-L^{am2}-, wherein each variable is as described herein. In some embodiments, each of L^{am1} and L^{am2} is optionally substituted bivalent C₁₋₆ aliphatic. In some embodiments, each of L^{am1} and L^{am2} is bivalent C₁₋₆ aliphatic. In some embodiments, each of L^{am} and L^{am2} is optionally substituted bivalent C₁₋₆ alkyl. In some embodiments, each of L^{am1} and L^{am2} is bivalent C₁₋₆ alkyl. In some embodiments, each of L^{am} and L^{am2} is optionally substituted bivalent linear C₁₋₆ alkyl. In some embodiments, each of L^{am} and L^{am2} is bivalent linear C₁₋₆ alkyl. In some embodiments, L^{am1} is —CH₂—. In some embodiments, L^{am2} is a covalent bond. In some embodiments, L^{am2} is —CH₂—. In some embodiments, both L^{am1} and L^{am2} are —CH₂—. In some embodiments, L^{am1} is —CH₂— and L^{am2} is a covalent bond. In some embodiments, —N(R′)— is —N(Et)-. In some embodiments, —N(R′)— is —N(CH₂CF₃)—. In some embodiments, L^{aa} is -L^{am1}-Cy-L^{am2}-, wherein each variable is as described herein. In some embodiments, -Cy- is optionally substituted phenyl. In some embodiments, -Cy- is optionally substituted 5-6 membered heteroaryl having 1-4 heteroatoms.

In some embodiments, a compound is

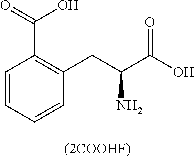 [SMILES: N[C@@H](Cc1ccccc1C(=O)O)C(=O)O]

or a salt thereof. In some embodiments, a compound is

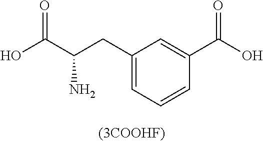 [SMILES: N[C@@H](Cc1cccc(C(=O)O)c1)C(=O)O]

or a salt thereof. In some embodiments, a compound is

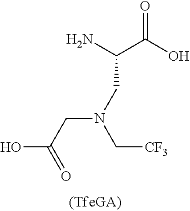 [SMILES: N[C@@H](CN(CC(=O)O)CC(F)(F)F)C(=O)O]

or a salt thereof. In some embodiments, a compound is

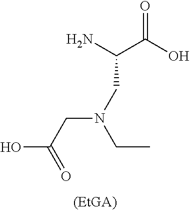

or a salt thereof. In some embodiments, a compound is

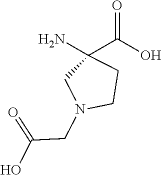 [SMILES: N[C@@]1(C(=O)O)CCN(CC(=O)O)C1]

or a salt thereof. In some embodiments, a compound is

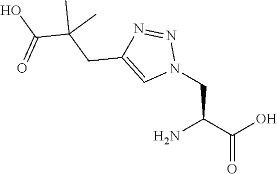 [SMILES: CC(C)(Cc1cn(C[C@H](N)C(=O)O)nn1)C(=O)O]

or a salt thereof. In some embodiments, a compound is

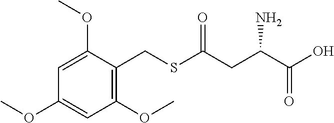 [SMILES: COc1cc(OC)c(CSC(=O)C[C@H](N)C(=O)O)c(OC)c1]

or a salt thereof. In some embodiments, a compound is

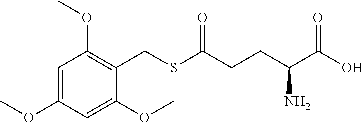 [SMILES: COc1cc(OC)c(CSC(=O)CC[C@H](N)C(=O)O)c(OC)c1]

or a salt thereof. In some embodiments, a compound is

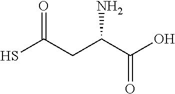

or a salt thereof. In some embodiments, a compound is

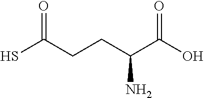

or a salt thereof. Among other things, such compounds may be utilized as amino acid residues in peptides including stapled peptides.

In some embodiments, the present disclosure provides a compound, e.g., a peptide, comprising a residue of a compound of formula PA or a salt form thereof. In some embodiments, a residue has the structure of —N(R^{a1})-L^{a1}-C(R^{a2})(R^{a3})-L^{a2}-C(O)— or a salt form thereof, wherein each variable is independently as described herein. In some embodiments, a residue has the structure of —N(R^{a1})-L^{a1}-C(-L^{aa}-COOH)(R^{a3})-L^{a2}-C(O)— or a salt form thereof, wherein each variable is independently as described herein. For example, in some embodiments, a residue is

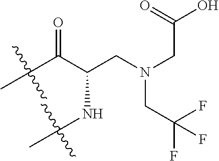 [SMILES: *N[C@@H](CN(CC(=O)O)CC(F)(F)F)C(*)=O]

or a salt form thereof. In some embodiments, a residue is

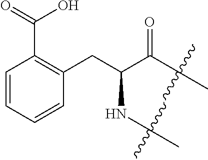 [SMILES: *N[C@@H](Cc1ccccc1C(=O)O)C(*)=O]

or a salt form thereof. In some embodiments, a residue is

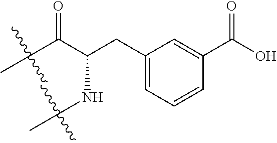 [SMILES: *N[C@@H](Cc1cccc(C(=O)O)c1)C(*)=O]

or a salt form thereof. In some embodiments, a residue is

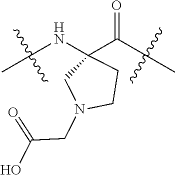 [SMILES: *N[C@@]1(C(*)=O)CCN(CC(=O)O)C1]

or a salt form thereof. In some embodiments, a residue is

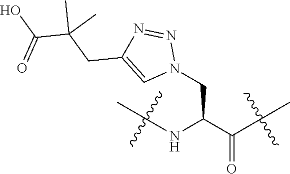 [SMILES: *N[C@@H](Cn1cc(CC(C)(C)C(=O)O)nn1)C(*)=O]

or a salt form thereof. In some embodiments, a residue is

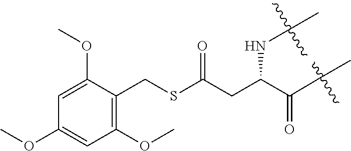 [SMILES: *N[C@@H](CC(=O)SCc1c(OC)cc(OC)cc1OC)C(*)=O]

or a salt form thereof. In some embodiments, a residue is

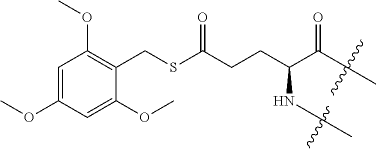 [SMILES: *N[C@@H](CCC(=O)SCc1c(OC)cc(OC)cc1OC)C(*)=O]

or a salt form thereof. In some embodiments, a residue is

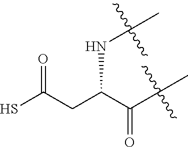 [SMILES: *N[C@@H](CC(=O)S)C(*)=O]

or a salt for thereof. In some embodiments, a residue is

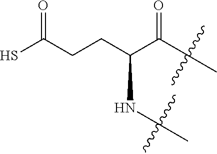 [SMILES: *N[C@@H](CCC(=O)S)C(*)=O]

or a salt form thereof.

Certain amino acids and structure moieties are described in WO 2022/020651 and WO 2022/020652, the amino acids and structure moieties of each of which are independently incorporated herein by reference, and can be utilized in accordance with the present disclosure

In some embodiments, an amino acid, or a structure moiety, of an amino acid or an agent (e.g., a peptide), is selected from below. A N-terminal cap (N-Term) is connected via R₁ to the amino group (R₁) of the first amino acid (AA1). In some embodiments, a N-Term cap may be properly considered as part of AA1. From there, each carboxylate (R₂) of that amino acid is connected to the amino group (R₁) of the subsequent amino acid, until the carboxylate (R₂) of the final amino acid is connected to R₁ of a C-terminal group. For any amino acid that has a branch point (R₃) and a branching monomer is indicated in brackets, R₁ of the monomer in brackets is attached to R₃ of the amino acid. For the amino acid Dap, with two potential branch points (R₃ and R₄), if two branches are indicated, the R₁ of the first branch is connected to R₃, and R₁ of the second branch connected to R₄. For any pair of amino acids that terminate in a *3 designation, the R₃ groups of each of those amino acids are linked to each other. Likewise, for any pair of amino acids that terminate in a **3 designation, the R₃ groups of those amino acids are linked to each other. For any sequence that contains a pair of branching amino acids with R₃ groups, and one contains a branching monomer that contains both R₁ and R₂ groups, then R₁ is attached to the branching amino acid adjacent to it in the sequence, and the R₂ group of the branching monomer is attached to R₃ of the amino acid with no branching monomer designated. For example, in various peptides that have one of Cys, hCys, Pen, or aMeC at position 10 and also one of Cys, hCys, Pen, or aMeC at position 14, and a branching group off of the amino acid residue 10, the R₁ of that branching group is tied to the R₃ of the amino acid residue at position 10, while the R₂ of that branching group is tied to the R₃ of the amino acid residue at position 14. For any amino acid which has a branching amino acid containing R₃ and nothing attached to it by the above, then R₃=H. Typically, all residues with terminal olefins are linked (stapled) by ring-closing metathesis. Certain examples are provided in Table E2 and Table E3. In some embodiments, the present disclosure provides agents, e.g., peptides such as stapled peptides, comprising one or more amino acid residues selected from below.

Table A-IV. Certain useful compounds or moieties.

TABLE A-IV
Certain useful compounds or moieties.

| Compound/ | |
|---|---|
| Bracket | |
| Moiety | Structure |
| Ala | 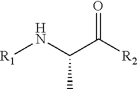 |
| Gly | 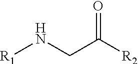 |
| Cys | 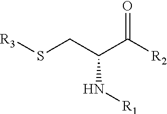 |
| His | 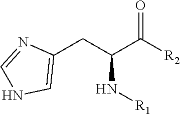 |
| Asp | 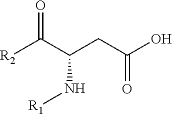 |
| Ile | 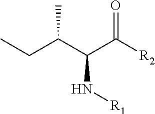 |
| Glu | 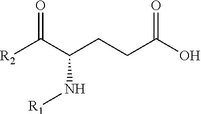 |
| Lys | 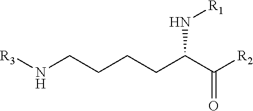 |
| Phe | 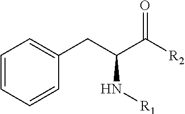 |
| Leu | 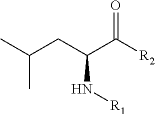 |
| nLeu | 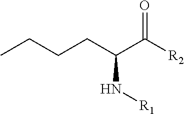 |
| Arg | 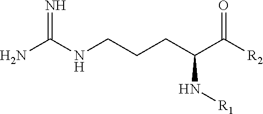 |
| Asn | 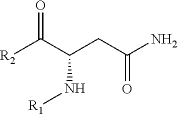 |
| Ser | 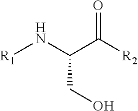 |
| Pro | 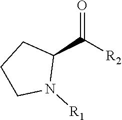 |
| Thr | 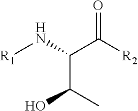 |
| Gln | 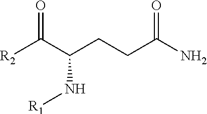 |
| aThr | 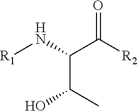 |
| GlnR | 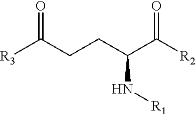 |
| Val | 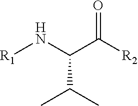 |
| Trp | 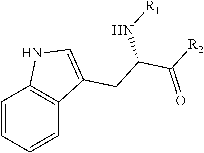 |
| 4F3MeF | 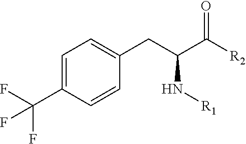 |
| Tyr | 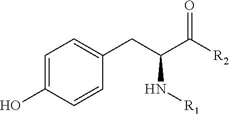 |
| Npg | 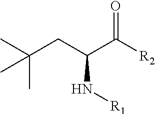 |
| MePro | 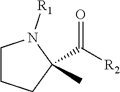 |
| Aib | 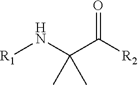 |
| PL3 | 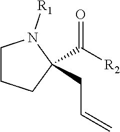 |
| Cpg | 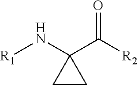 |
| B5 | 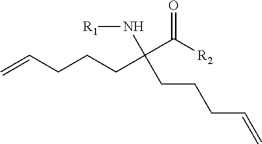 |
| Cbg | 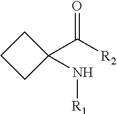 |
| PyrS2 | 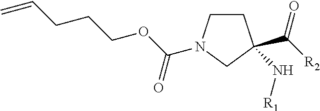 |
| CyLeu | 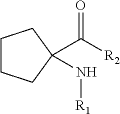 |
| BztA | 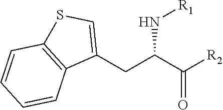 |
| Orn | 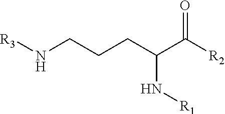 |
| 3Thi | 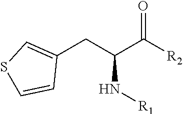 |
| Dab | 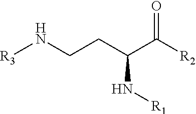 |
| 2Thi | 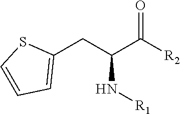 |
| TriAzLys | 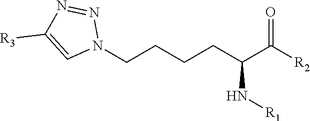 |
| 2F3MeF | 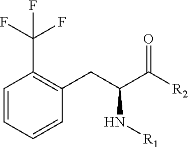 |
| TriAzOrn | 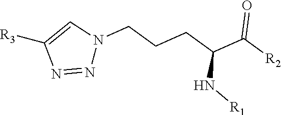 |
| sAla | 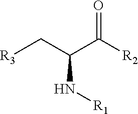 |
| TfeGA | 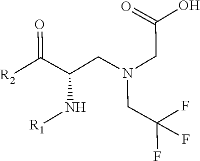 |
| sAbu | 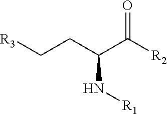 |
| iPrLys | 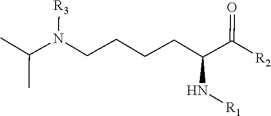 |
| sCH2S | 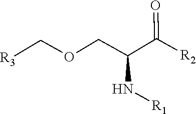 |
| MeAsn | 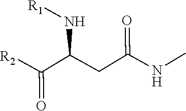 |
| dLys | 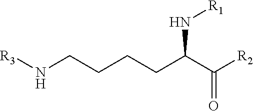 |
| hGlnR | 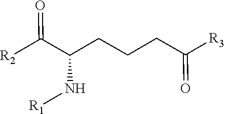 |
| dOrn | 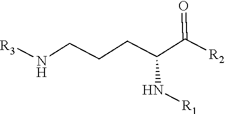 |
| 2OH3COOHF | 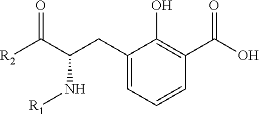 |
| DGlnR | 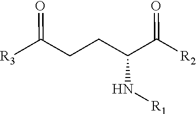 |
| 4OH3COOHF | 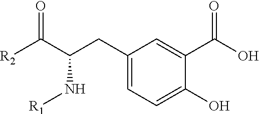 |
| DAsnR | 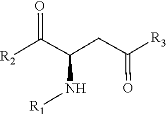 |
| TriAzDap | 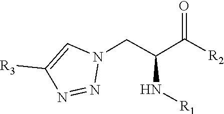 |
| 3COOHF | 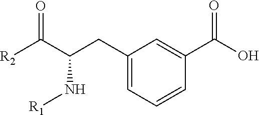 |
| 4COOHF | 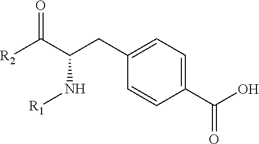 |
| Hse | 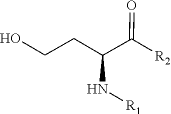 |
| 2COOHF | 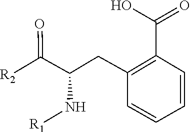 |
| Cha | 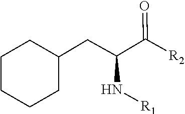 |
| 5F3Me2COOHF | 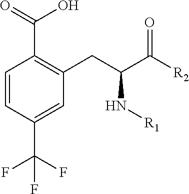 |
| 4F3Me2COOHF | 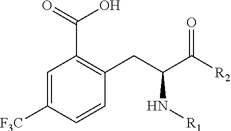 |
| Cba | 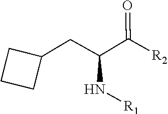 |
| 5F3Me3COOHF | 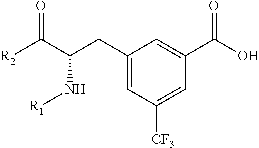 |
| SbMeAsp | 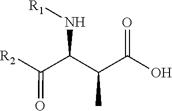 |
| 4F3Me3COOHF | 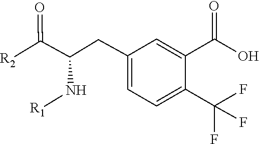 |
| RbMeAsp | 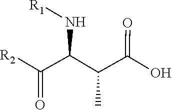 |
| 3F2COOHF | 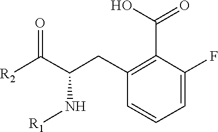 |
| 2FurA | 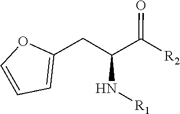 |
| dGlu | 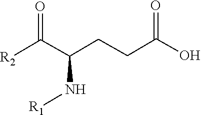 |
| 2OMeF | 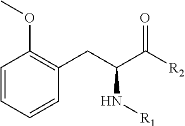 |
| hTyr | 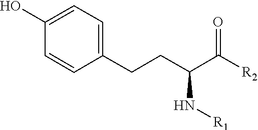 |
| 2MeF | 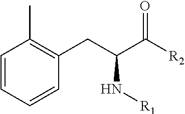 |
| 3cbmf | 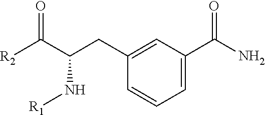 |
| 2BrF | 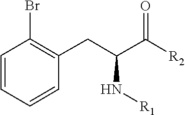 |
| MorphNva | 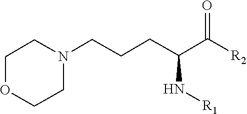 |
| 2ClF | 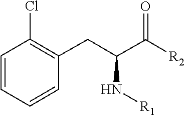 |
| R4 | 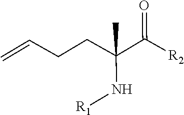 |
| 2CNF | 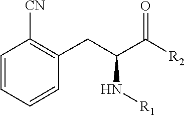 |
| R5 | 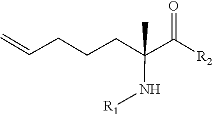 |
| 2NO2F | 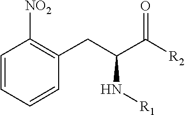 |
| R6 | 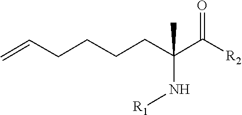 |
| 2PyrA | 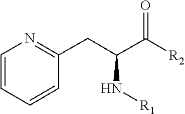 |
| CypA | 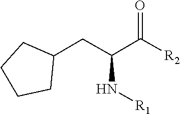 |
| 3PyrA | 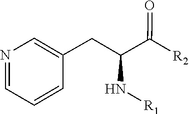 |
| Chg | 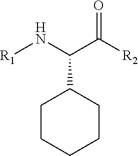 |
| 4PryA | 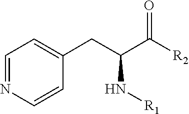 |
| Pff | 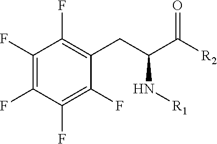 |
| 3BrF | 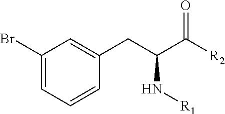 |
| DiethA | 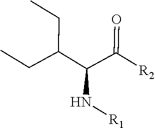 |
| 34MeF | 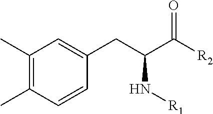 |
| 4PipA | 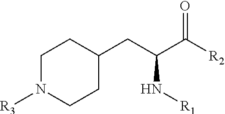 |
| 34ClF | 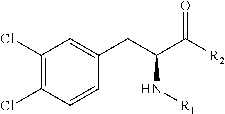 |
| Abu | 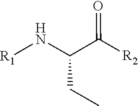 |
| Phg | 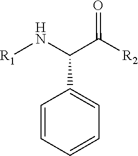 |
| Nva | 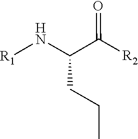 |
| DipA | 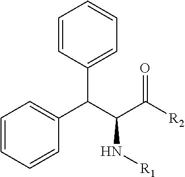 |
| hLeu | 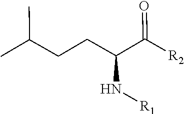 |
| OctG | 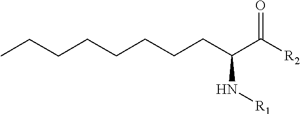 |
| Cpa | 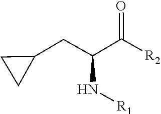 |
| F2PipNva | 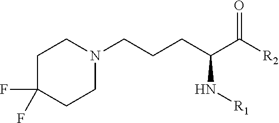 |
| MorphGln | 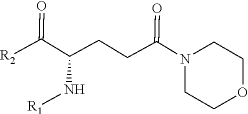 |
| Aad | 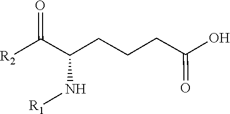 |
| 1NapA | 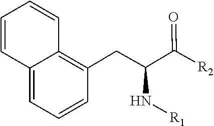 |
| hPhe | 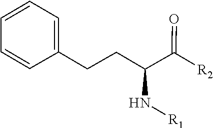 |
| 2NapA | 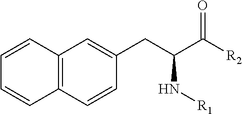 |
| hnLeu | 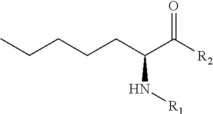 |
| Me2Gln | 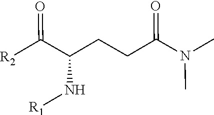 |
| 2cbmf | 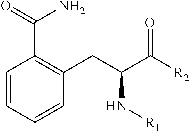 |
| AcLys | 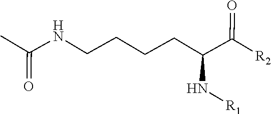 |
| dOrn | 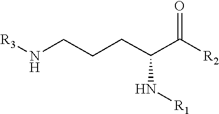 |
| Met2O | 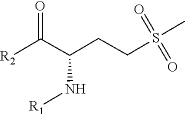 |
| dDab | 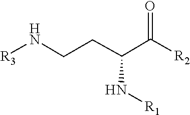 |
| Acp | 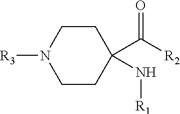 |
| MeOrn | 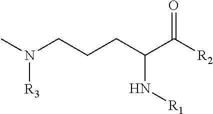 |
| 2Cpg | 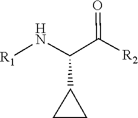 |
| Dap | 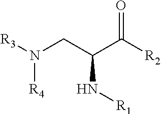 |
| aMeL | 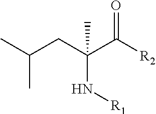 |
| 4FF | 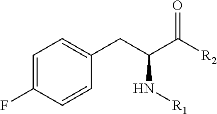 |
| DaMeL | 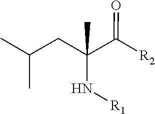 |
| 4ClF | 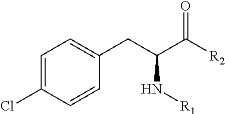 |
| aMeV | 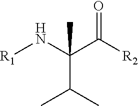 |
| 4BrF | 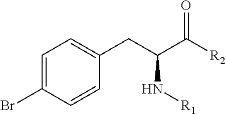 |
| aMeS | 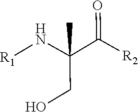 |
| 4CNF | 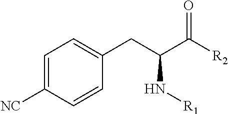 |
| DaMeS | 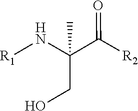 |
| 4MeF | 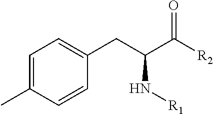 |
| aMeF | 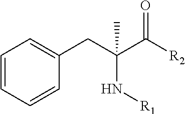 |
| 3FF | 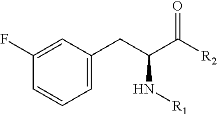 |
| aMeDF | 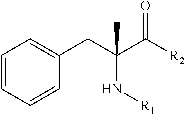 |
| 3ClF | 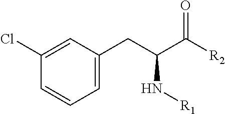 |
| dAla | 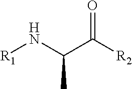 |
| 3BrF | 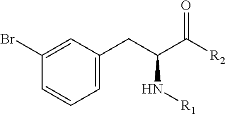 |
| dLeu | 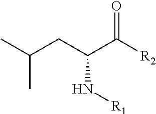 |
| 3OMeF | 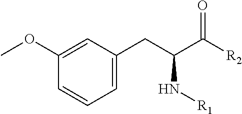 |
| OAsp | 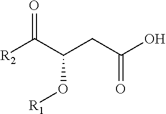 |
| 3MeF | 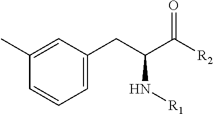 |
| Sar | 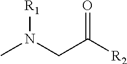 |
| 3CNF | 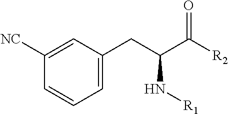 |
| NMebAla | 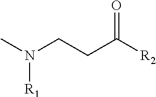 |
| 2FF | 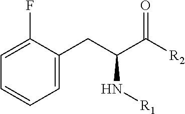 |
| Aic | 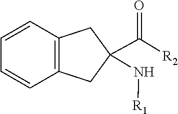 |
| [BzAm2OAllyl] | 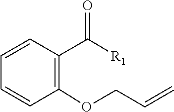 |
| RbiPrF | 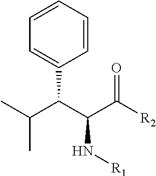 |
| [oXyl] | 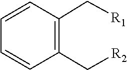 |
| SbiPrF | 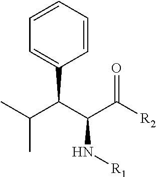 |
| [mXyl] | 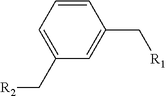 |
| RbiPrDF | 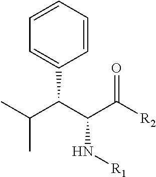 |
| [pXyl] | 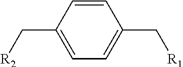 |
| RbMeXylA | 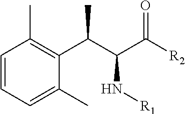 |
| [4FB] | 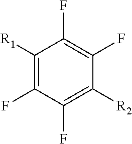 |
| RbMeXylDA | 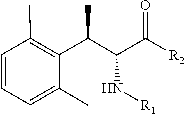 |
| [8FBB] | 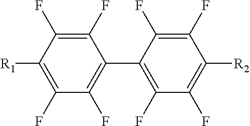 |
| SbMeXylA | 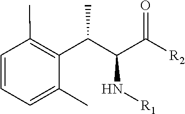 |
| [CH2CMe2CO2H] | 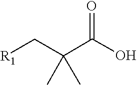 |
| SbMeXylDA | 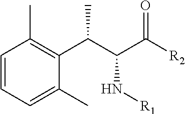 |
| [NHEt] | 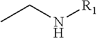 |
| AzLys | 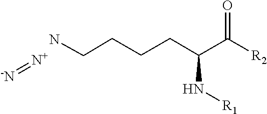 |
| [29N2spiro- undecane] | 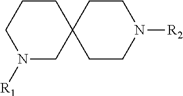 |
| AllylGly | 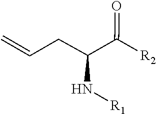 |
| [39N2spiro- undecane] | 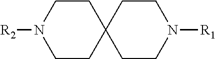 |
| [CyCO] | 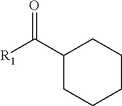 |
| [4mampiperidine] | 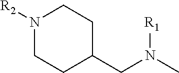 |
| [Piv] | 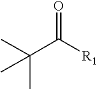 |
| [4aminopiperidine] | 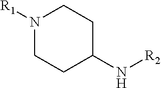 |
| [Phc] | 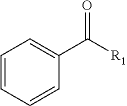 |
| [diaminobutane] | 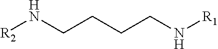 |
| [Bn] | 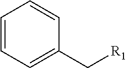 |
| NH2 | 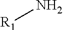 |
| [3butenyl] | 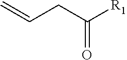 |
| OH | 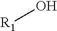 |
| [Allyl] | 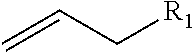 |
| dAlaol | 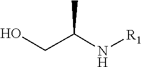 |
| [5hexenyl] | 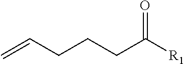 |
| Alaol | 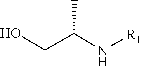 |
| [4pentenyl] | 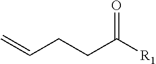 |
| Serol | 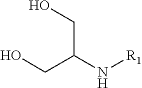 |
| [3_3-biph] | 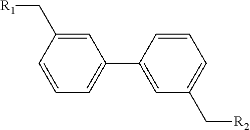 |
| Prool | 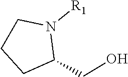 |
| [2_6-naph] | 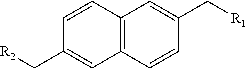 |
| Throl | 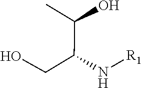 |
| 4TriA | 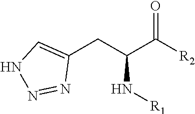 |
| [2COOH4NH2Ph] | 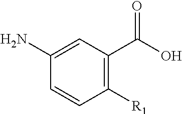 |
| 3F3MeF | 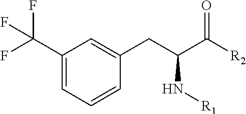 |
| [2COOH4NO2Ph] | 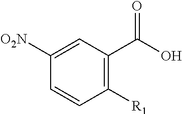 |
| AsnR | 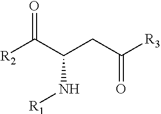 |
| [2COOHPh] | 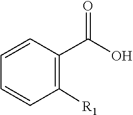 |
| aMeDAsp | 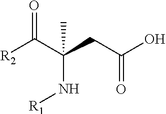 |
| [2Nic] | 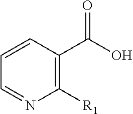 |
| [isophthalate] | 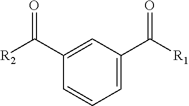 |
| [2OxoPpz] | 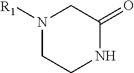 |
| [succinate] | 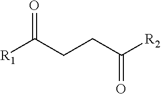 |
| [3C] | 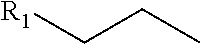 |
| [Me2Mal] | 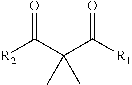 |
| [3Py] | 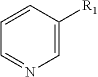 |
| [diphenate] | 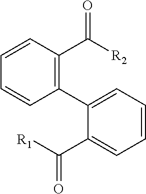 |
| [4AcMePip] | 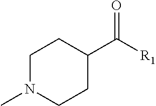 |
| [Biphen33COOH] | 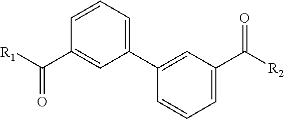 |
| [4CF3PhAc] | 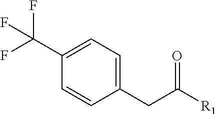 |
| ThioPro | 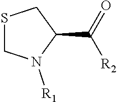 |
| [4F3CPip] | 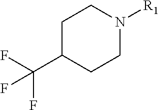 |
| [4MePpzPip] | 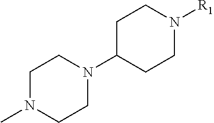 |
| [EtSSEt] | 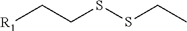 |
| [4Pippip] | 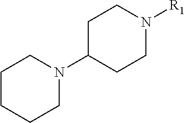 |
| [EtSSHex] | 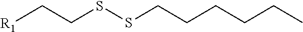 |
| [4PyPip] | 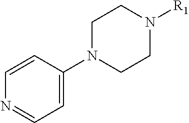 |
| [EtSSPh] | 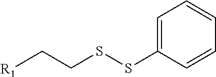 |
| [Ac] | 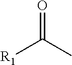 |
| [EtSSpy] | 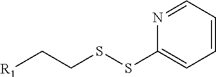 |
| [AcPpz] | 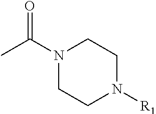 |
| [H4IAP] | 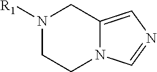 |
| [bismethoxy- ethylamine] | 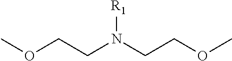 |
| [isoindoline] | 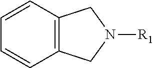 |
| [Bn] | 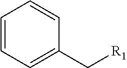 |
| [lithocholate] | 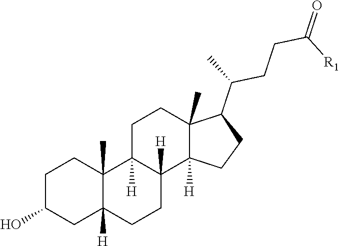 |
| [CCpCO2H] | 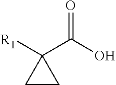 |
| [PEG2] | 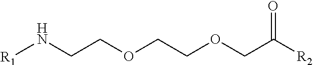 |
| [CF3CO] | 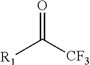 |
| [Me] | 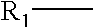 |
| [CH2CChCO2H] | 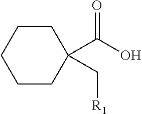 |
| [Me2diamino- butane] | 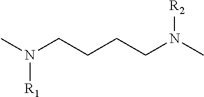 |
| [CH2CCpCO2H] | 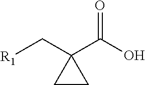 |
| [Me2NCBz] | 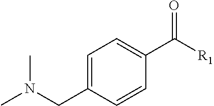 |
| [CH2CH2CO2H] | 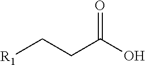 |
| [Me2Npr] | 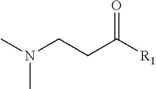 |
| [CH2CMe2CO2H] | 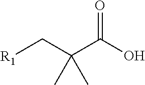 |
| [Me2NPrPip] | 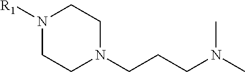 |
| [CH2CO2H] | 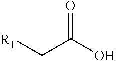 |
| [Me3AdamantC] | 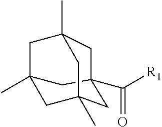 |
| [CH2NMe2] | 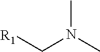 |
| [MeMorphBz] | 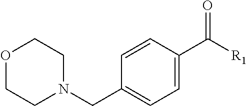 |
| [CH2Ppz] | 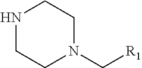 |
| [MePipAc] | 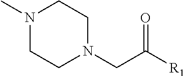 |
| [CMe2CO2H] | 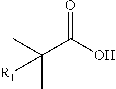 |
| [MeSO2] | 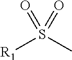 |
| [CyPr] | 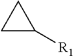 |
| [Morph] | 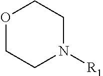 |
| [Et] | 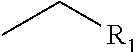 |
| [MorphAc] | 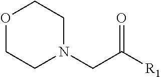 |
| [EtSO2Ppz] | 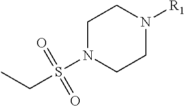 |
| [MorphCH2] | 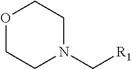 |
| [MorphEt] | 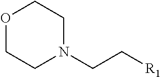 |
| 2F3MeW | 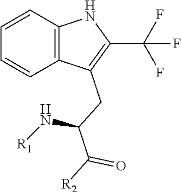 |
| [NdiMeButC] | 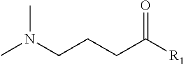 |
| 2NH2F | 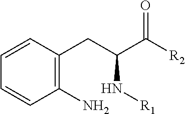 |
| [NHBn] | 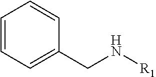 |
| 34ClF | 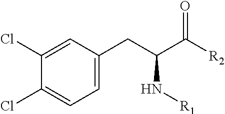 |
| [NHEt] | 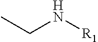 |
| 34MeF | 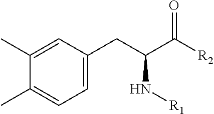 |
| [NMe2] | 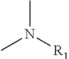 |
| 3Br4FF | 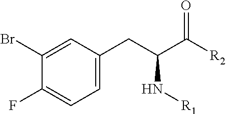 |
| [PfbGA] | 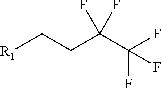 |
| 3BrF | 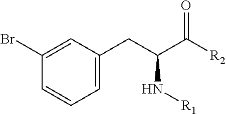 |
| [Pfbn] | 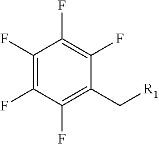 |
| 3CBMF | 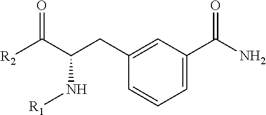 |
| [PfBz] | 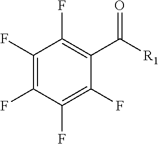 |
| 3CH2NMe2F | 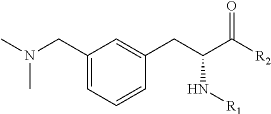 |
| [PfPhAc] | 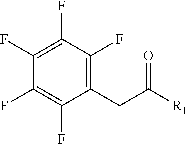 |
| 3CO2PhF | 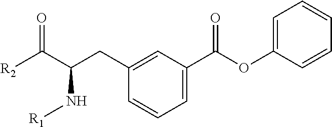 |
| [Ph] | 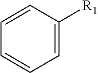 |
| 3SF | 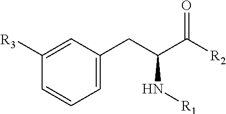 |
| [Phc] | 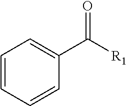 |
| 3SO2F | 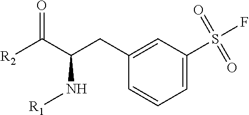 |
| [Pic] | 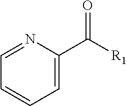 |
| 3TzF | 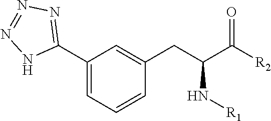 |
| [Ppz] | 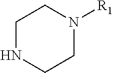 |
| 4BrF | 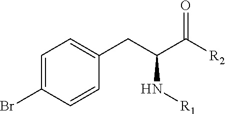 |
| [RDMAPyr] | 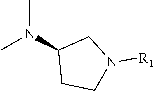 |
| 4ClBztA | 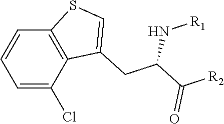 |
| [Red] | 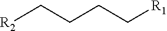 |
| 4ClW | 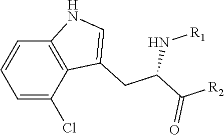 |
| [sBu] | 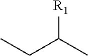 |
| 4F3COOHF | 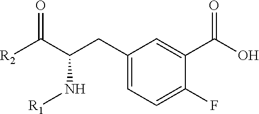 |
| [SO2MorphCH2] | 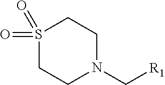 |
| 4FW | 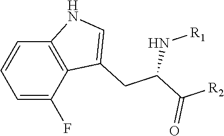 |
| [Tfb] | 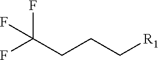 |
| 4SEF | 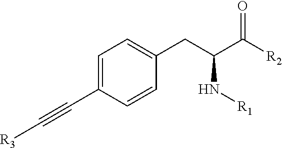 |
| [TfePpz] | 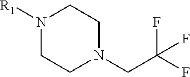 |
| 4TzF | 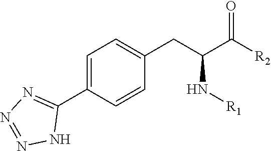 |
| [Tfp] | 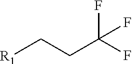 |
| 5F3Me3COOHF | 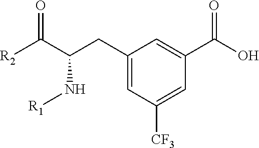 |
| 5IndA | 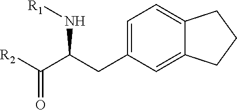 |
| BzAm3Oallyl | 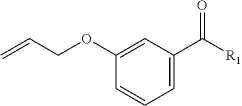 |
| 5iPr3COOHF | 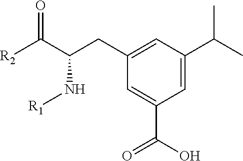 |
| Cba | 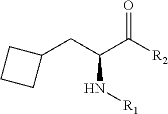 |
| 7AzaW | 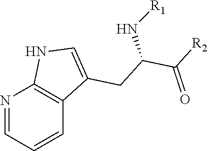 |
| Cbg | 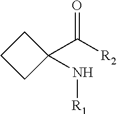 |
| 7ClBztA | 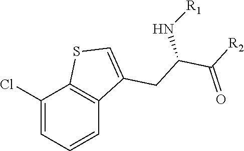 |
| ClAc | 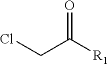 |
| 7FBztA | 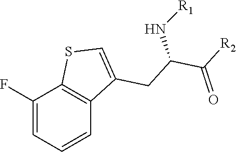 |
| CO | 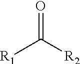 |
| AcAsp | 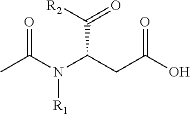 |
| CO2Bu | 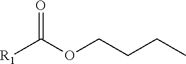 |
| AcLys | 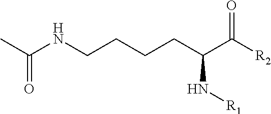 |
| CO2Hex | 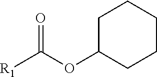 |
| AspE | 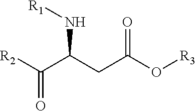 |
| CO2iBu | 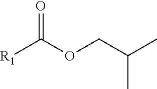 |
| AspSH | 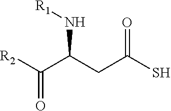 |
| CO2Me | 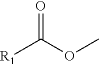 |
| Az2 | 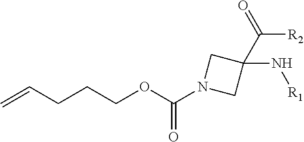 |
| CO2Ph | 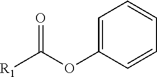 |
| Az3 | 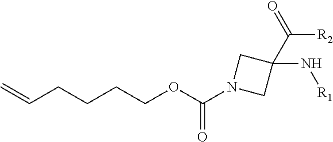 |
| Cpg | 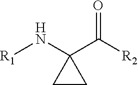 |
| B3 | 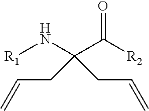 |
| CyLeu | 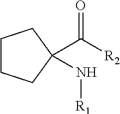 |
| B4 | 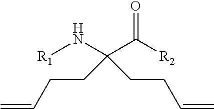 |
| dAla | 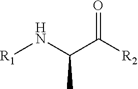 |
| B6 | 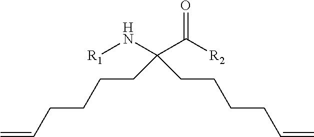 |
| dIle | 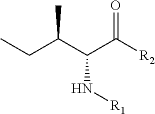 |
| bMe2Asp | 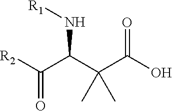 |
| dLeu | 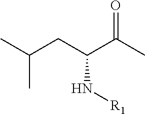 |
| Bn3OAllyl | 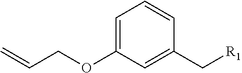 |
| F2PipAbu | 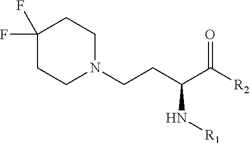 |
| BnBoroleK | 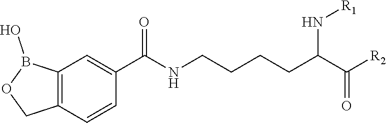 |
| F2PipNva | 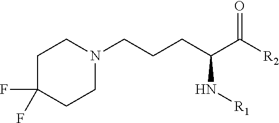 |
| Bnc | 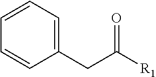 |
| GA | 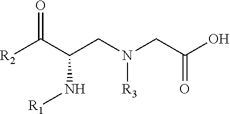 |
| BrAc | 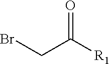 |
| GAbu | 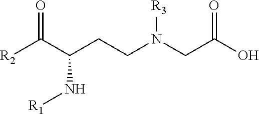 |
| BzAm2Allyl | 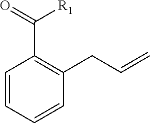 |
| GlnR | 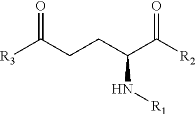 |
| GluE | 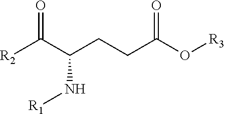 |
| NMebAla | 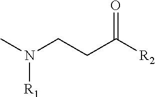 |
| GluSH | 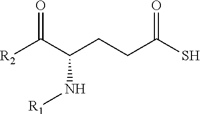 |
| Npa | 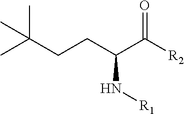 |
| hhLeu | 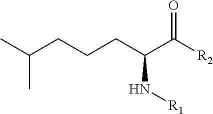 |
| PAc3OAllyl | 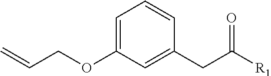 |
| HypBzEs3OAllyl | 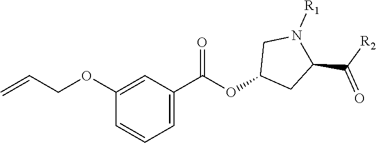 |
| ProAm5 | 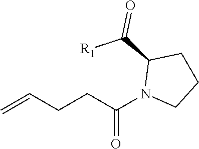 |
| HypEs4 | 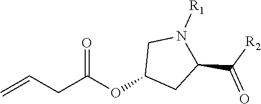 |
| ProAm6 | 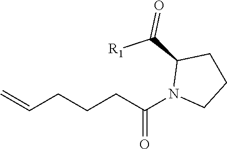 |
| HypEs5 | 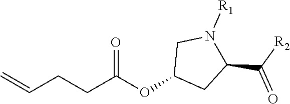 |
| ProBzAm3OAllyl | 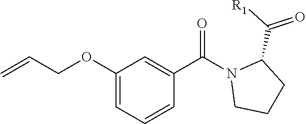 |
| HypPAc3OAllyl | 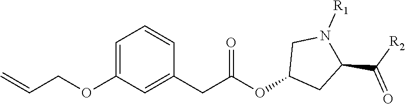 |
| ProPAc3OAllyl | 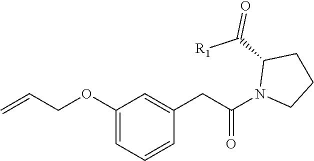 |
| Me2Asn | 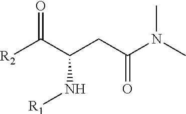 |
| PropynOH | 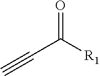 |
| Me2Gln | 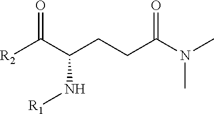 |
| ProSAm3 | 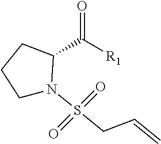 |
| MeAsn | 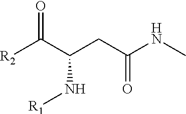 |
| PyrR | 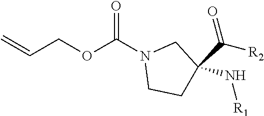 |
| MeGln | 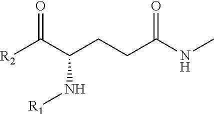 |
| PyrR2 | 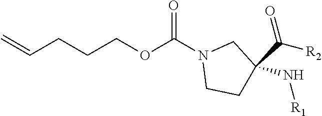 |
| MePpzAbu | 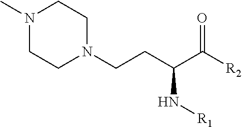 |
| PyrS4 | 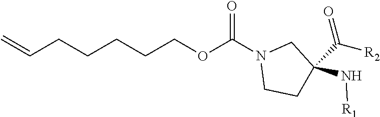 |
| MePpzAsn | 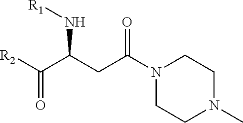 |
| R2COOPipA | 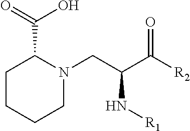 |
| MePpzNva | 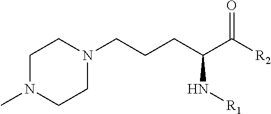 |
| R3COOPipA | 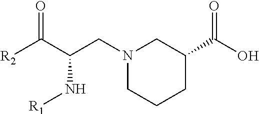 |
| MePro | 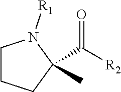 |
| RbMe2NapA | 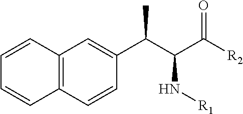 |
| Met2O | 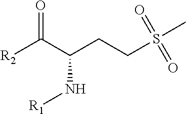 |
| RbMeBztA | 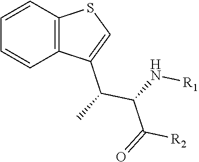 |
| MorphAbu | 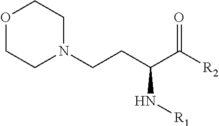 |
| RbOHAsp | 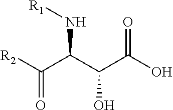 |
| MorphAsn | 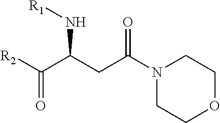 |
| S2COOPipA | 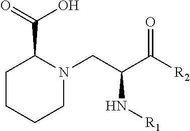 |
| MorphGln | 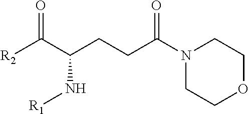 |
| S3COOPipA | 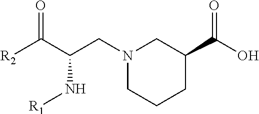 |
| MorphNva | 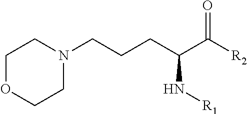 |
| sAc | 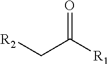 |
| sAla | 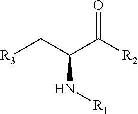 |
| SPip2 | 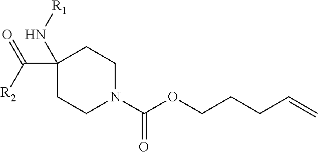 |
| Sar | 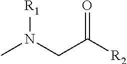 |
| SPip3 | 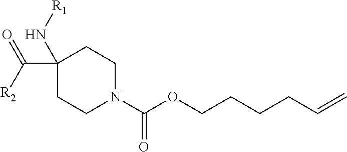 |
| SbMe2NapA | 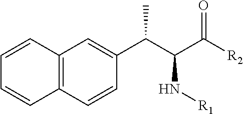 |
| sPr | 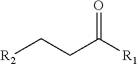 |
| SbMeBztA | 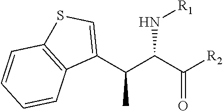 |
| TriAzDap | 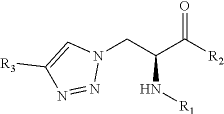 |
| sBut | 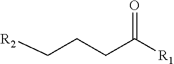 |
| TriAzDab | 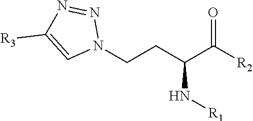 |
| SeNc5 | 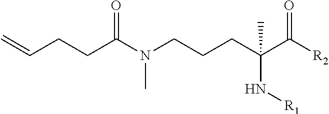 |
| TriAzLys | 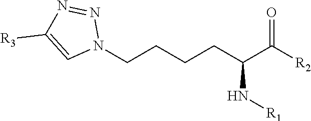 |
| SPip1 | 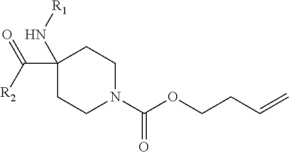 |
| TriAzdLys | 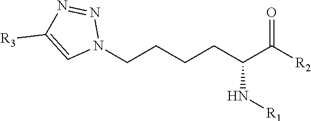 |
| BiotinPEG8 | 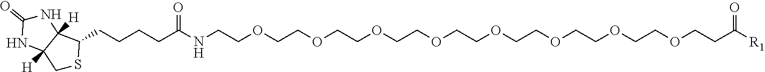 |

TABLE A-IV
(Continued; certain moieties may be presented in [ ])
Certain moieties useful as, e.g., Lys analogs, branch point amino
acid residues, or non-RCM stapling amino acid residues

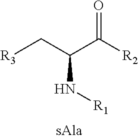

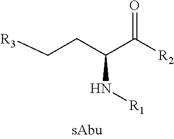

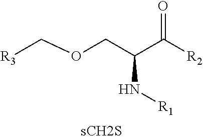

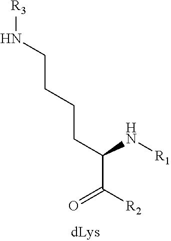

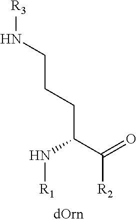

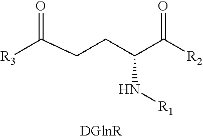

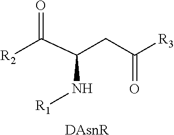

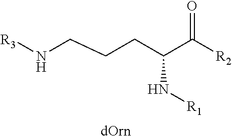

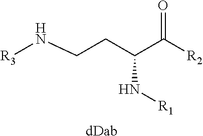

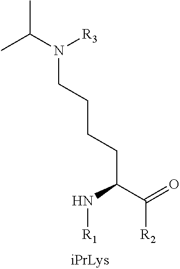

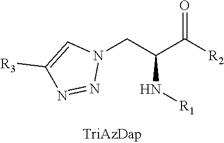

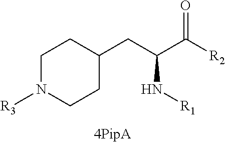

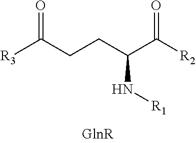

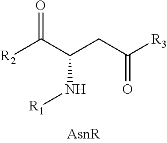

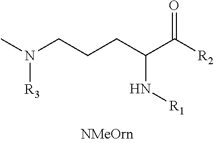

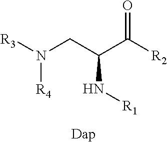

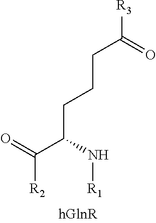

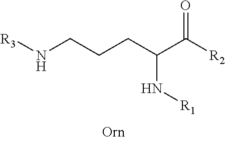

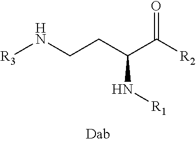

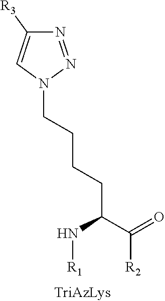

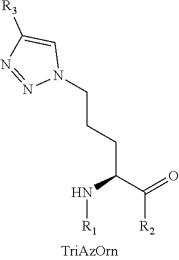

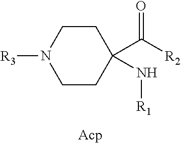

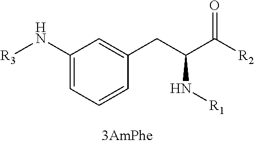

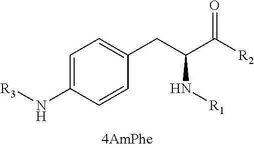

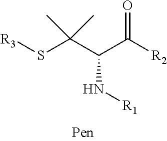

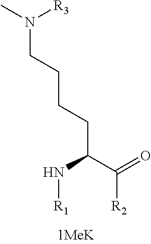

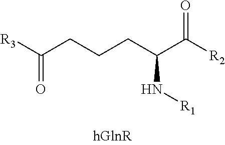

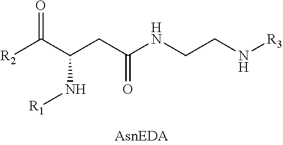

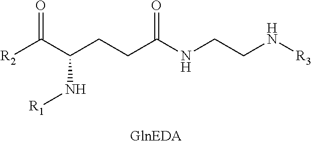

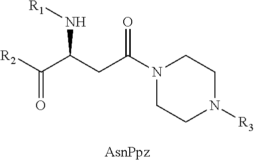

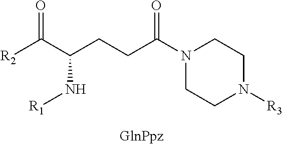

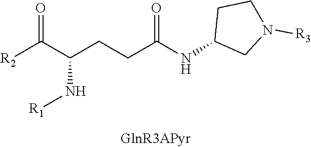

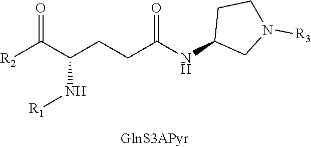

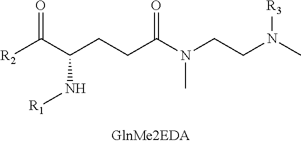

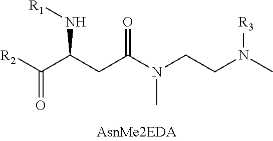

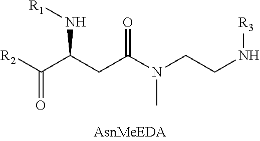

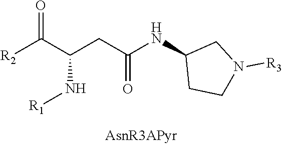

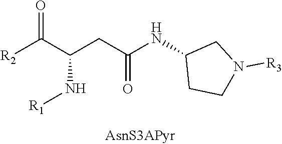

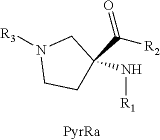

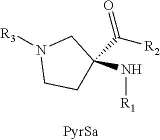

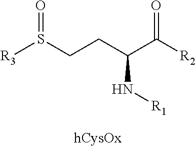

Certain moieties useful as, e.g., stapling amino acid residues (e.g., RCM for other stapling technologies)

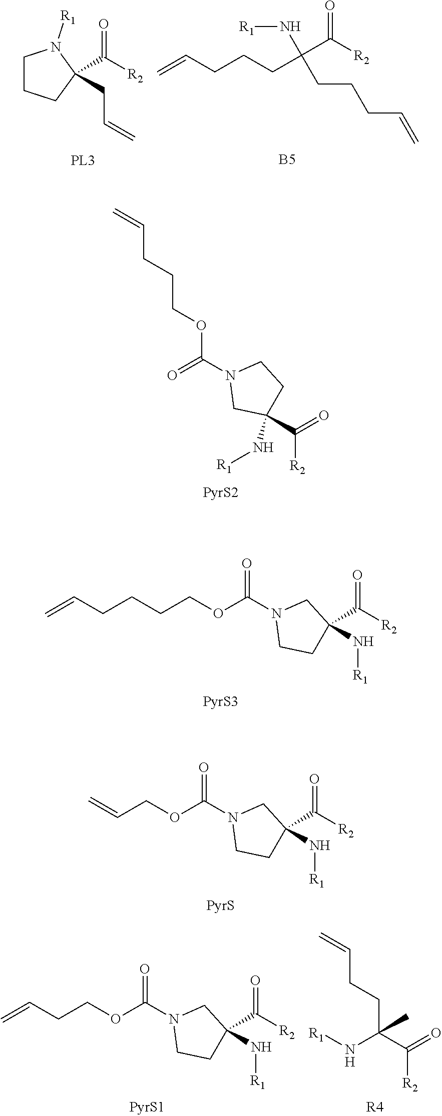

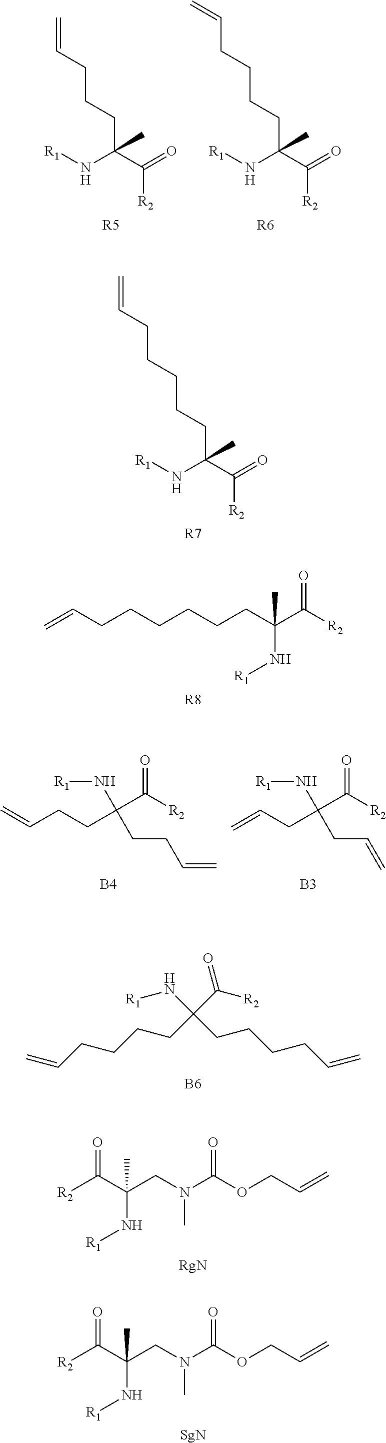

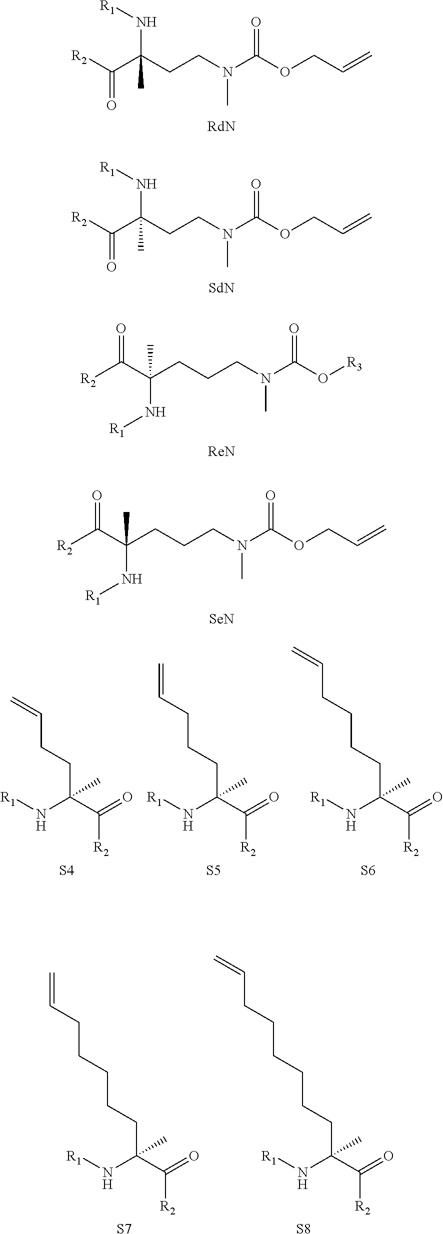

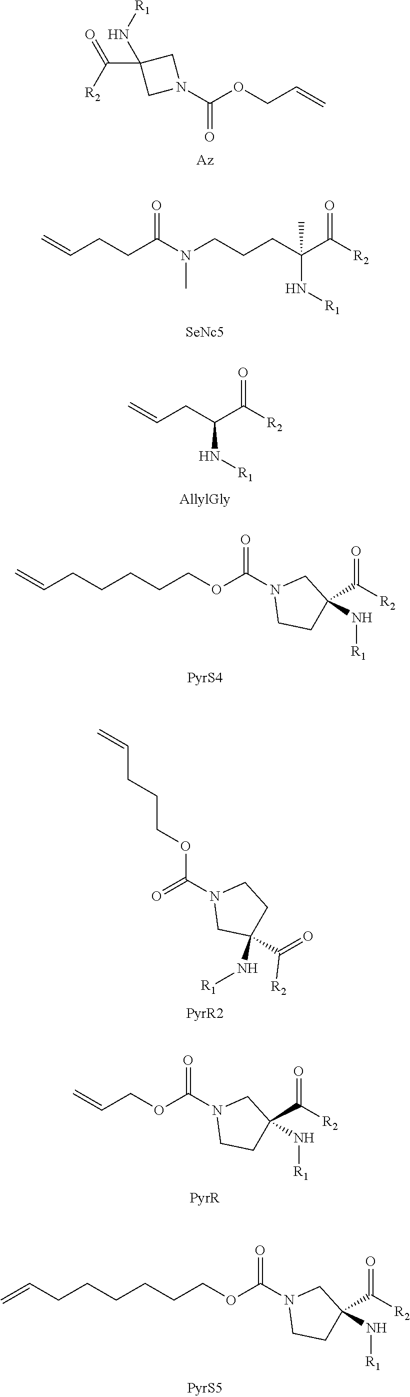

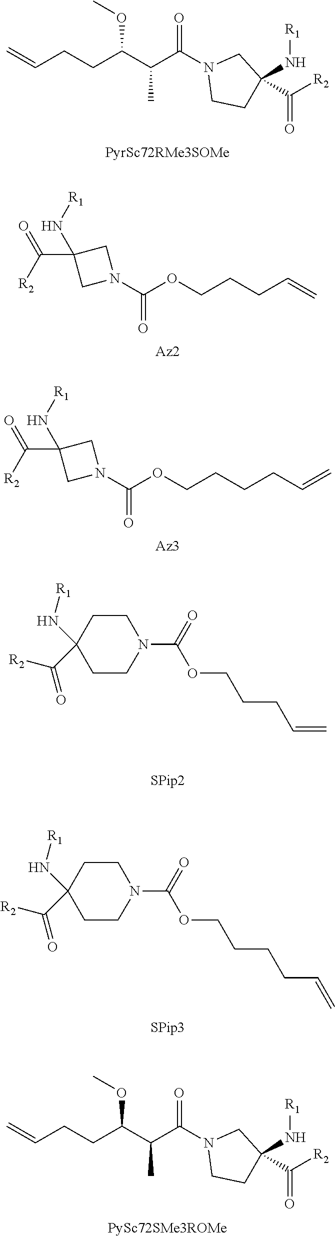

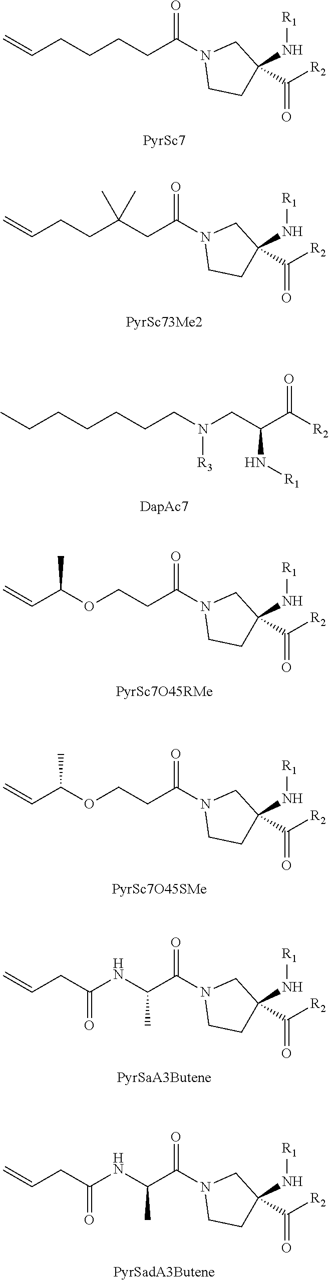

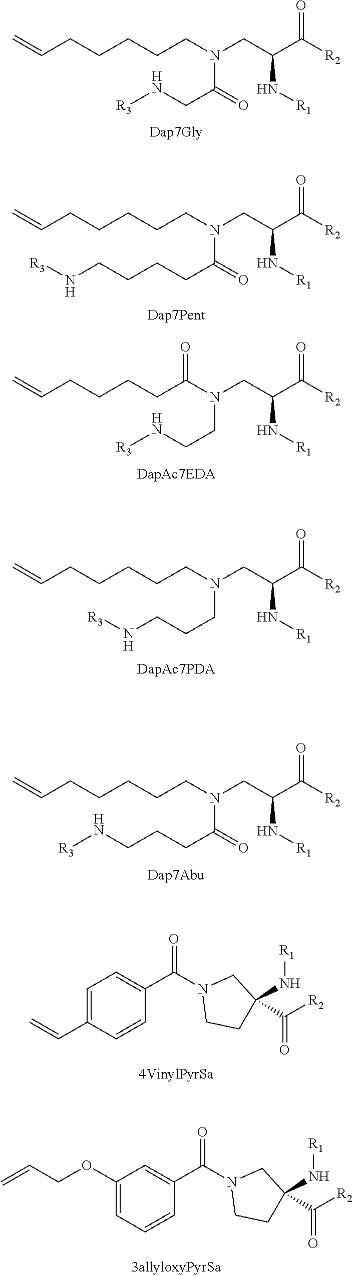

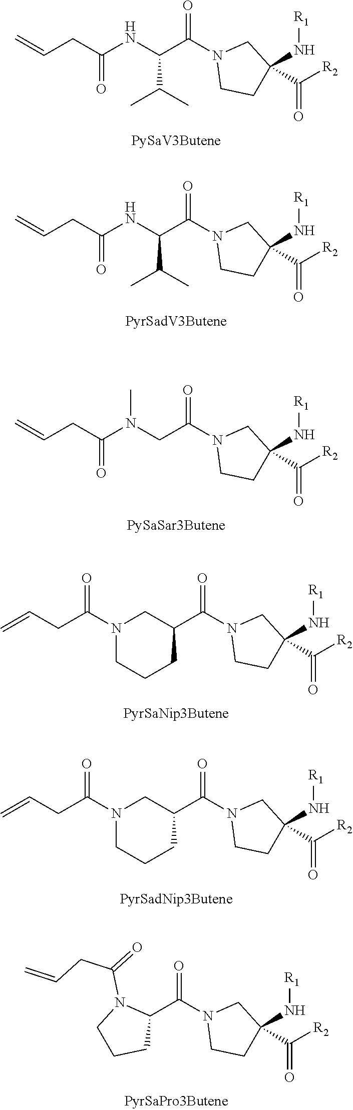

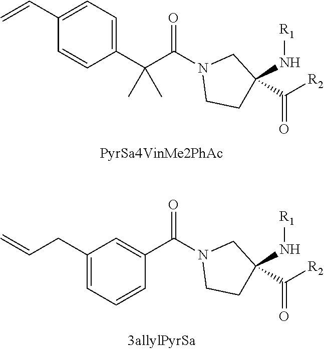

Certain moieties useful as, e.g., aromatic amino acid residues

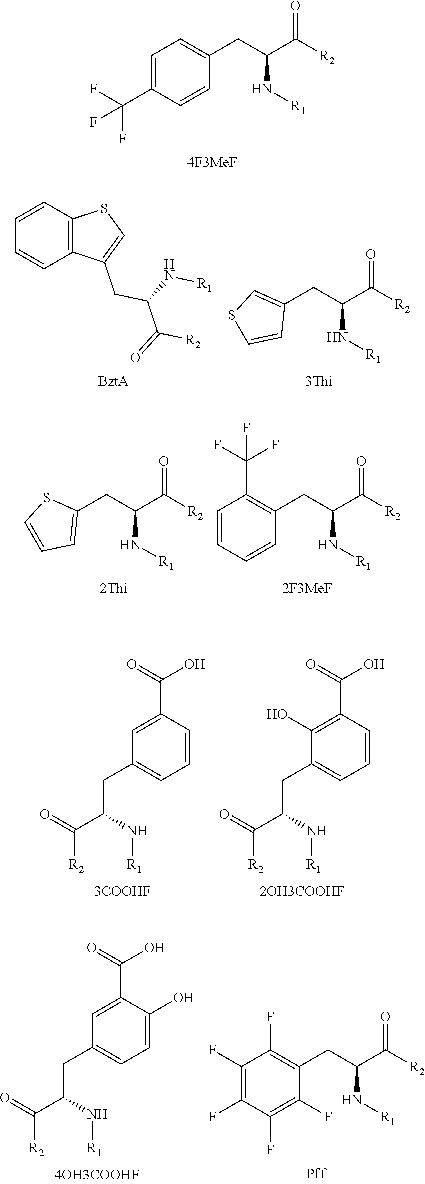

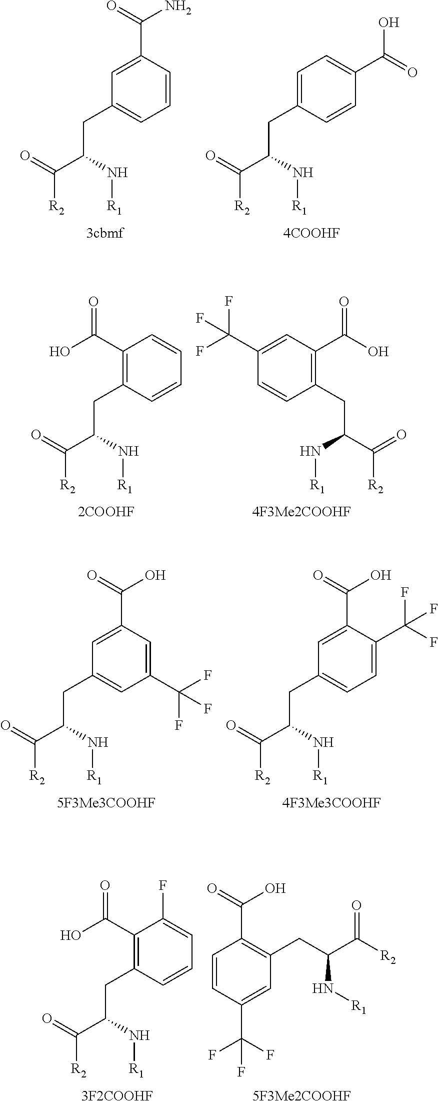

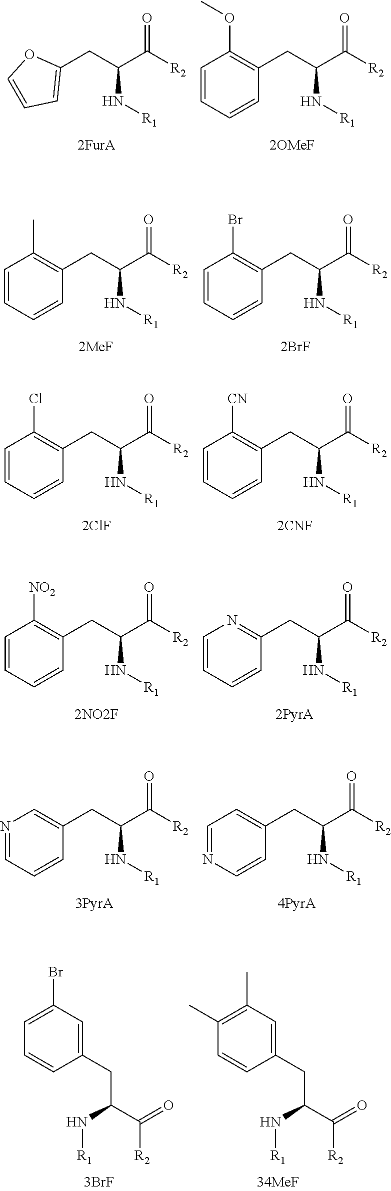

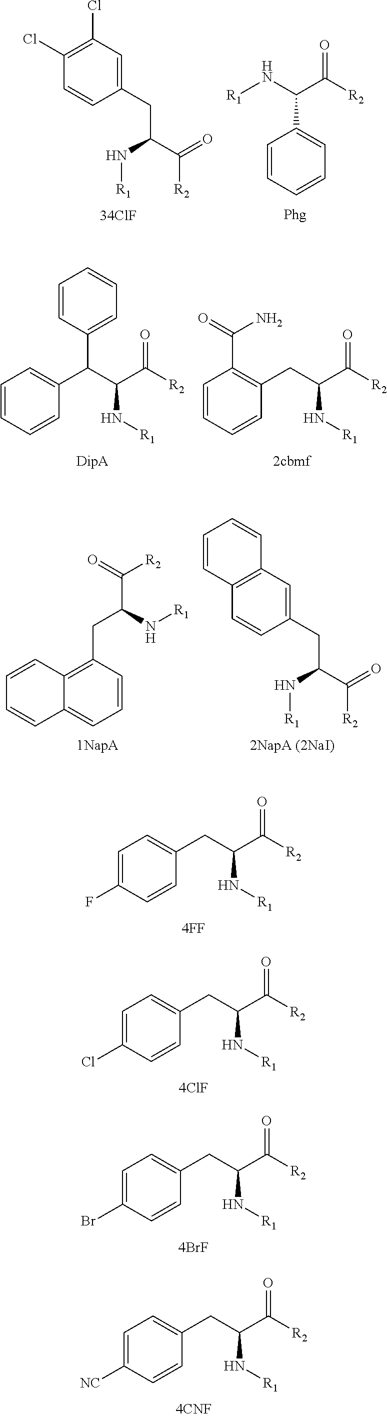

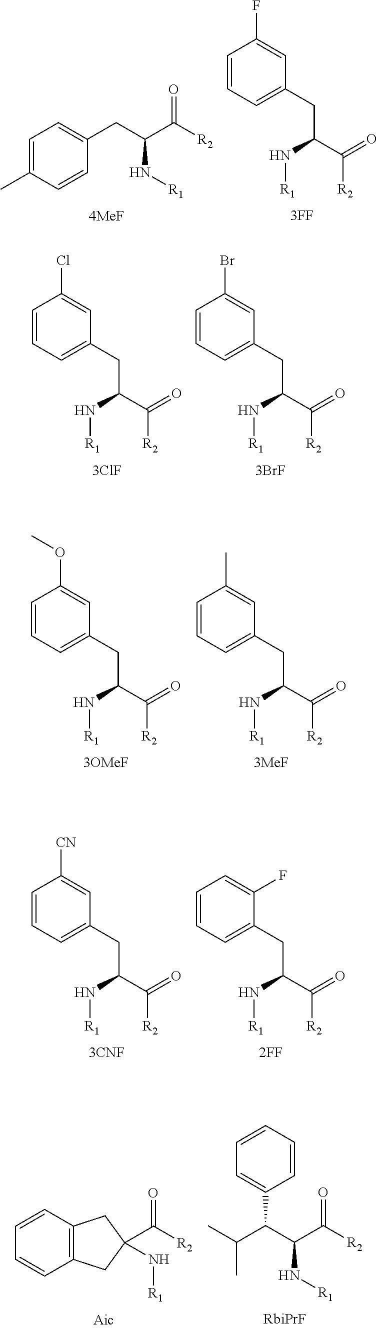

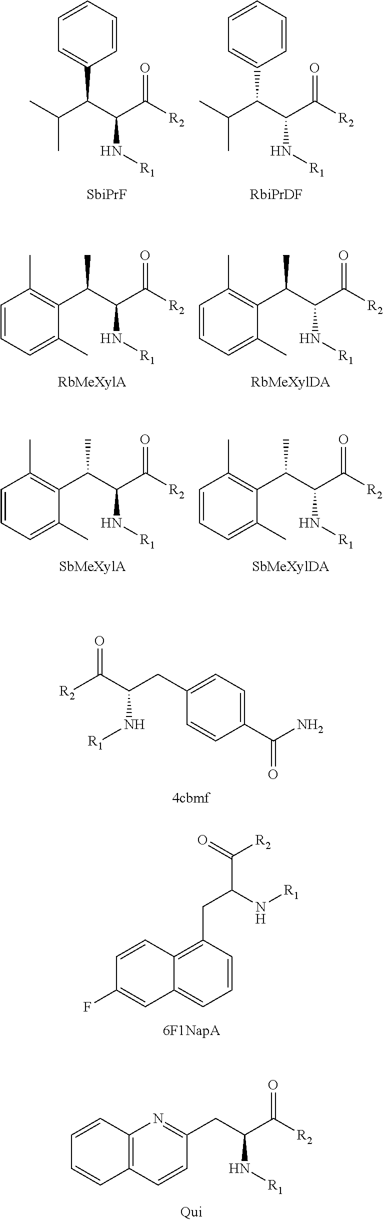

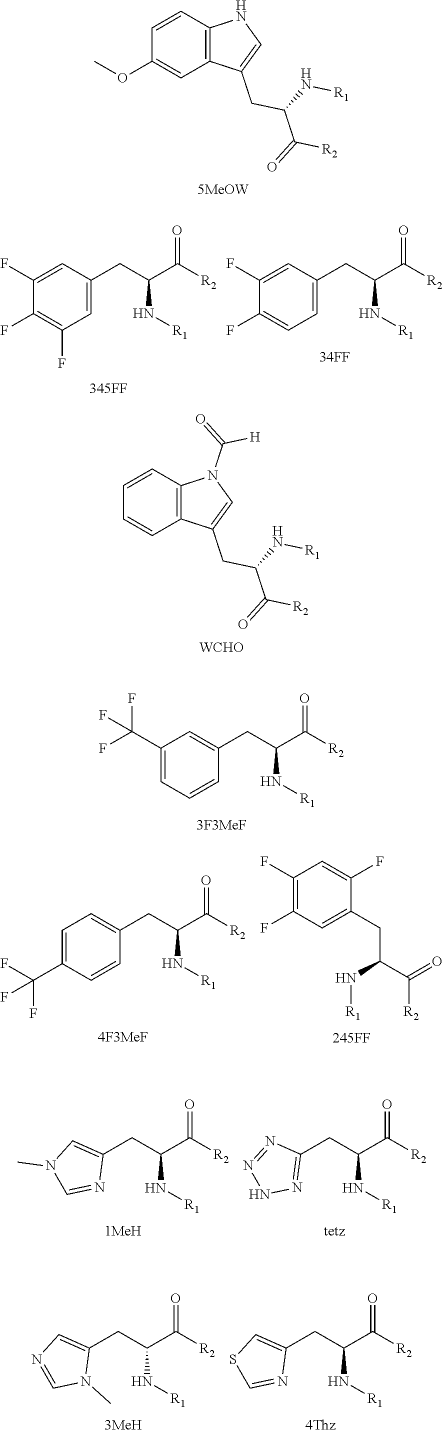

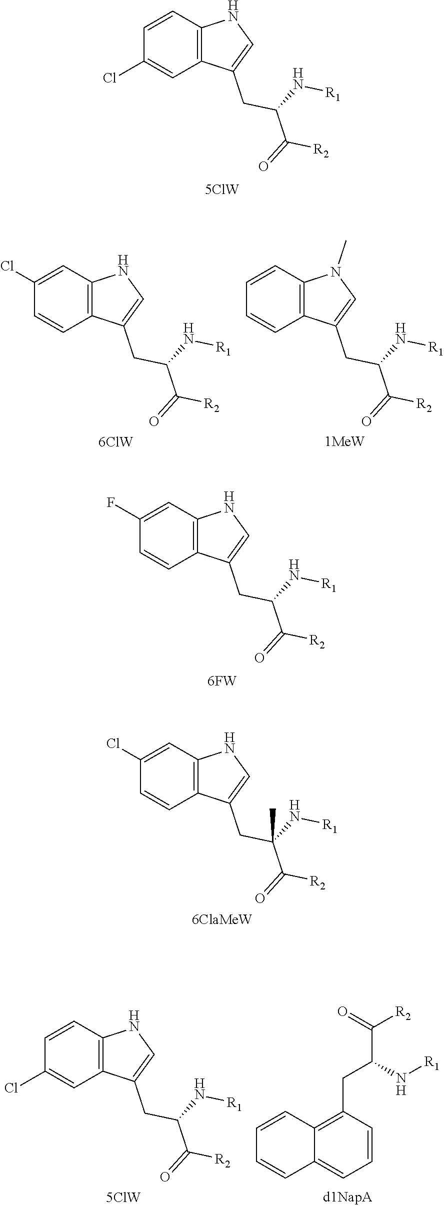

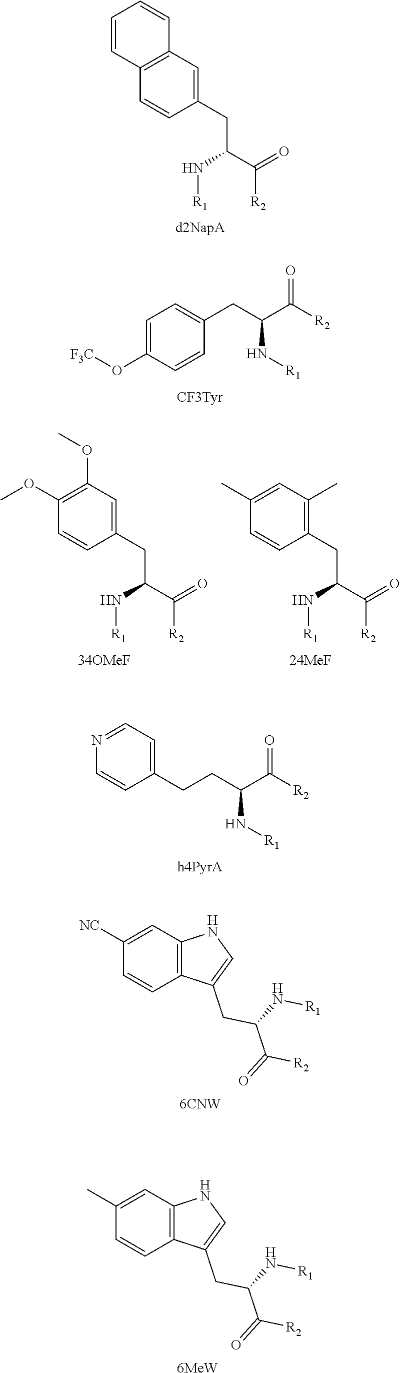

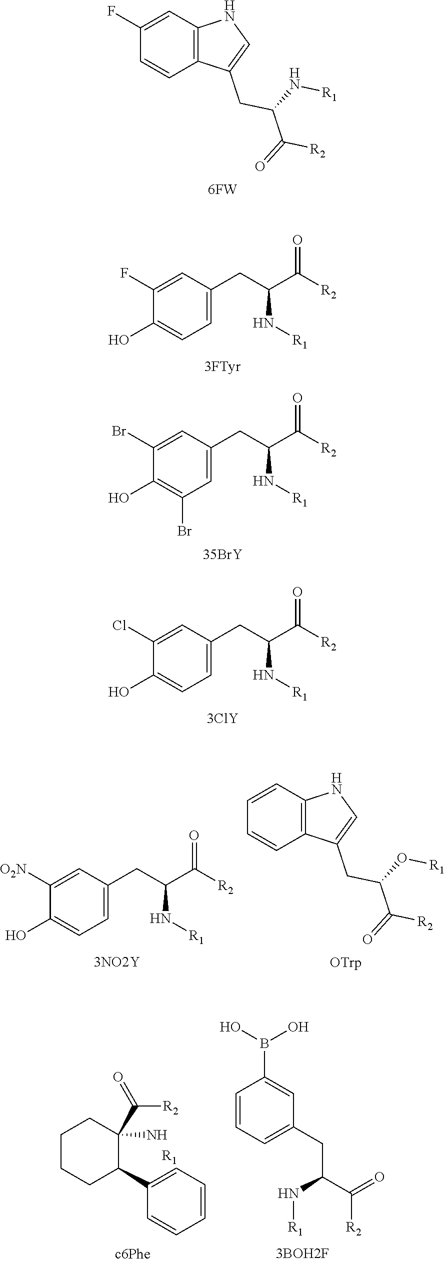

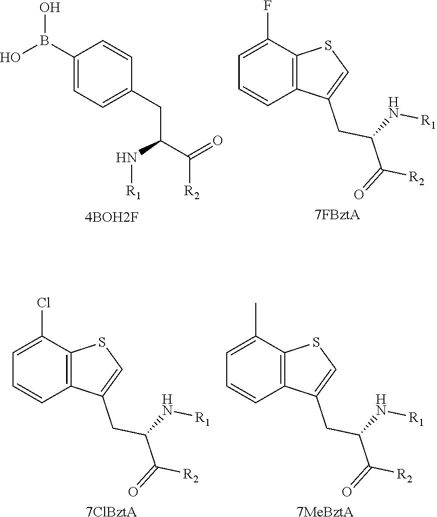

Certain moieties useful as amino acid residues

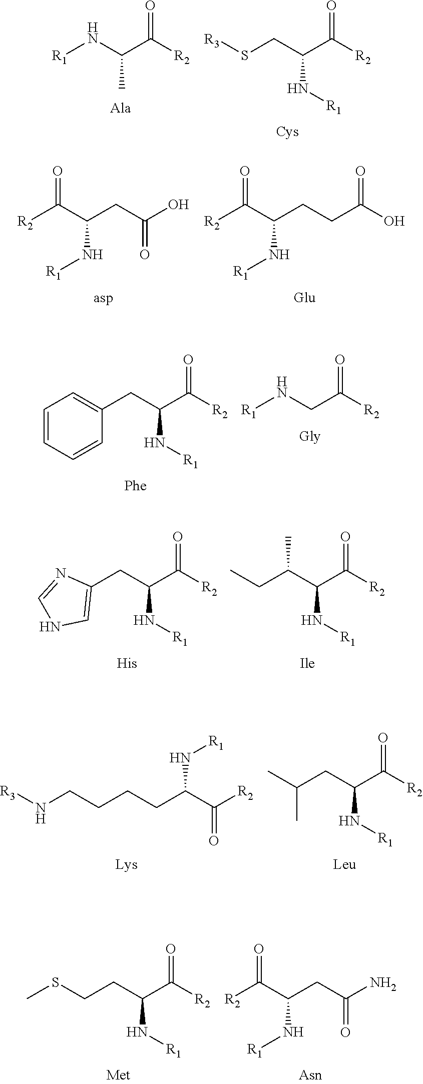

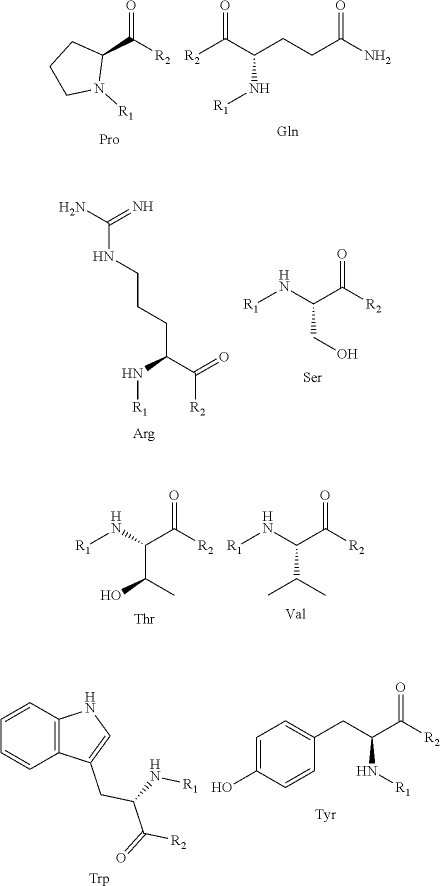

Certain moieties useful as, e.g., amino acid residues (e.g., D-amino acid residues, homologated amino acid residues, alkyl (e.g., methyl) amino acid residues, etc.)

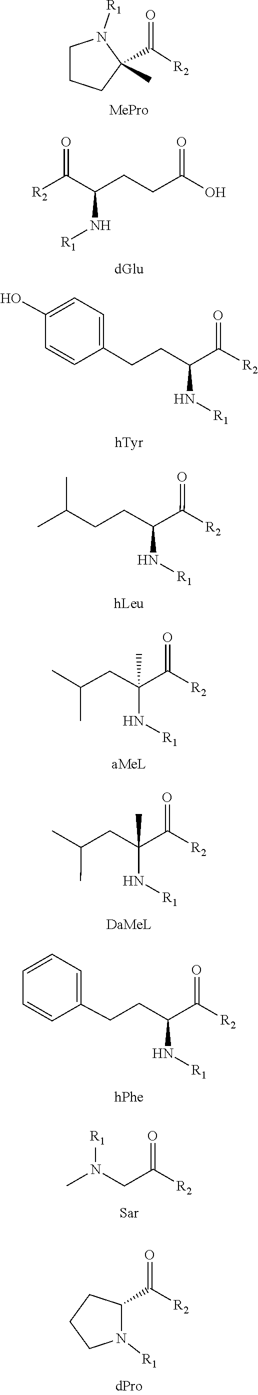

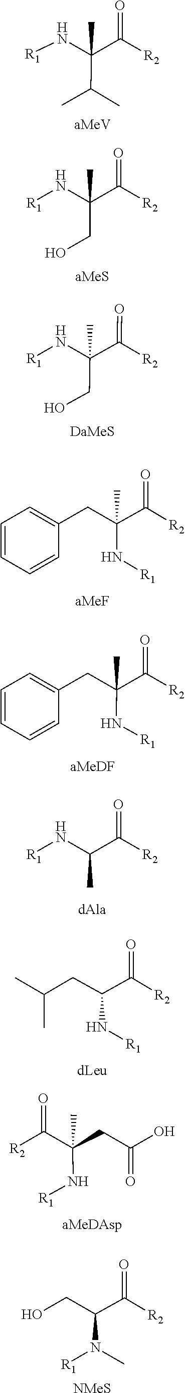

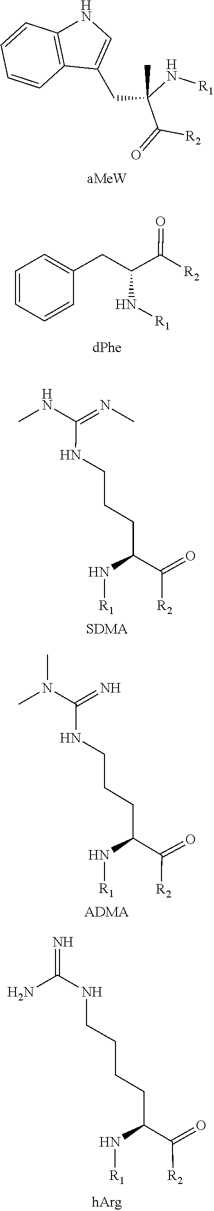

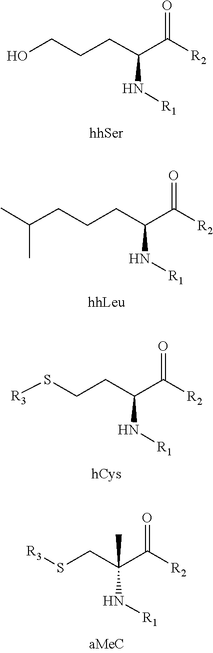

Certain moieties useful as, e.g., amino acid residues (e.g., alkyl amino acid residues, hydrophobic amino acid residues, etc.)

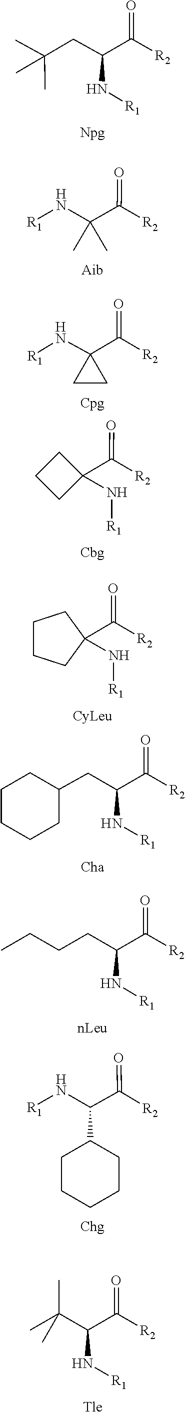

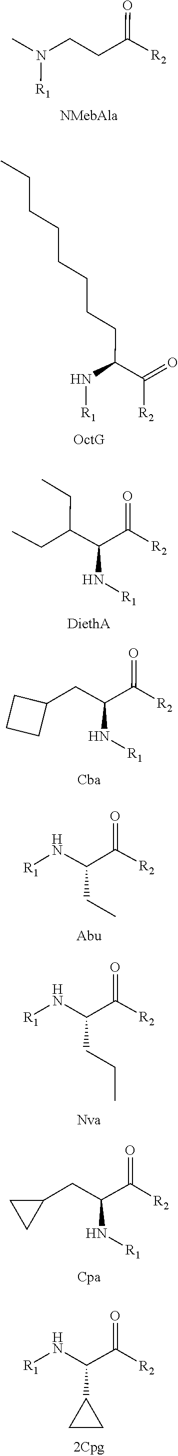

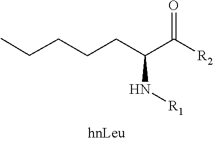

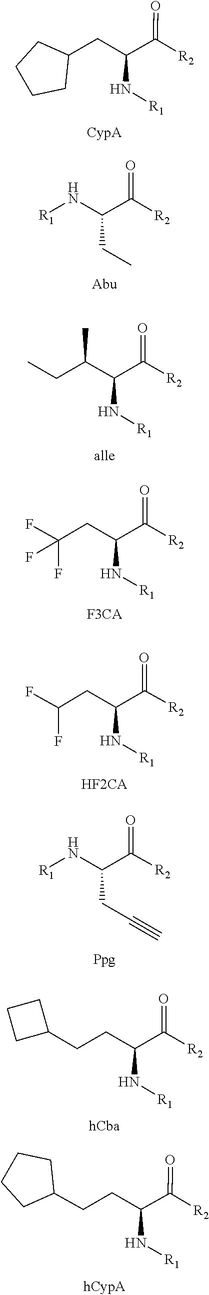

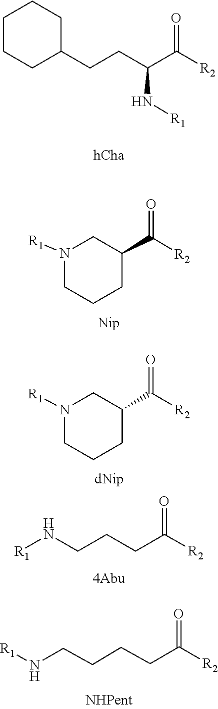

Certain moieties useful as, e.g., amino acid residues (e.g., polar amino acid residues, basic amino acid residues, etc.)

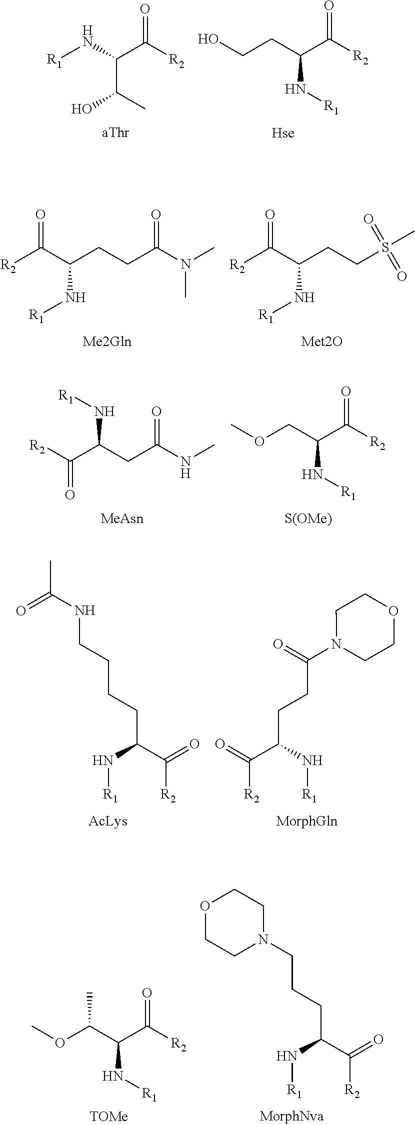

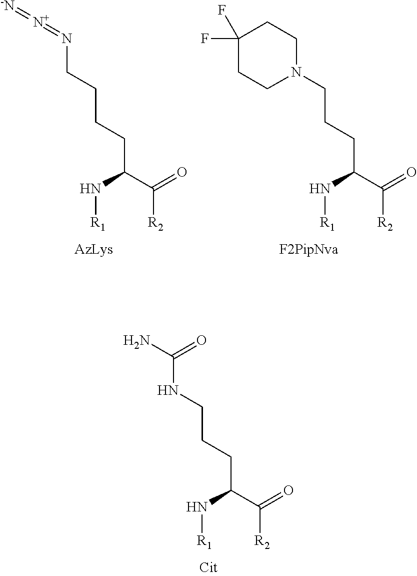

Certain moieties useful as, e.g., amino acid residues (e.g., acidic amino acid residues, non-aromatic amino acid residues, etc.)

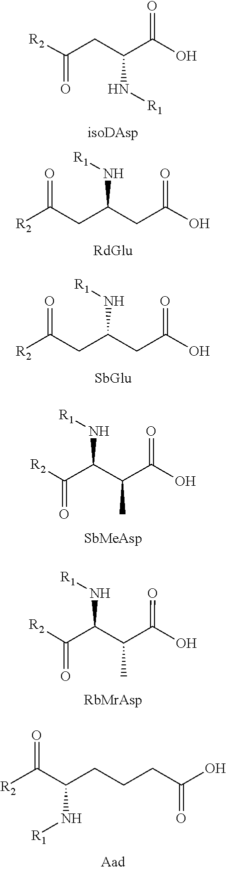

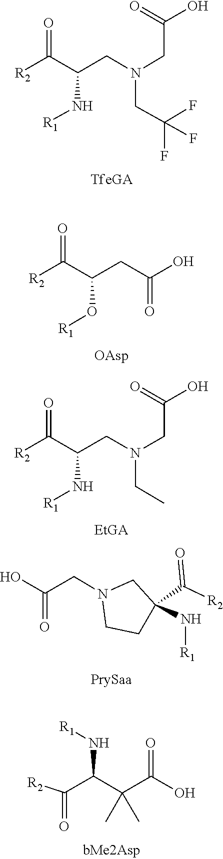

Certain moieties (e.g., moieties utilized in [ ] in various agents)

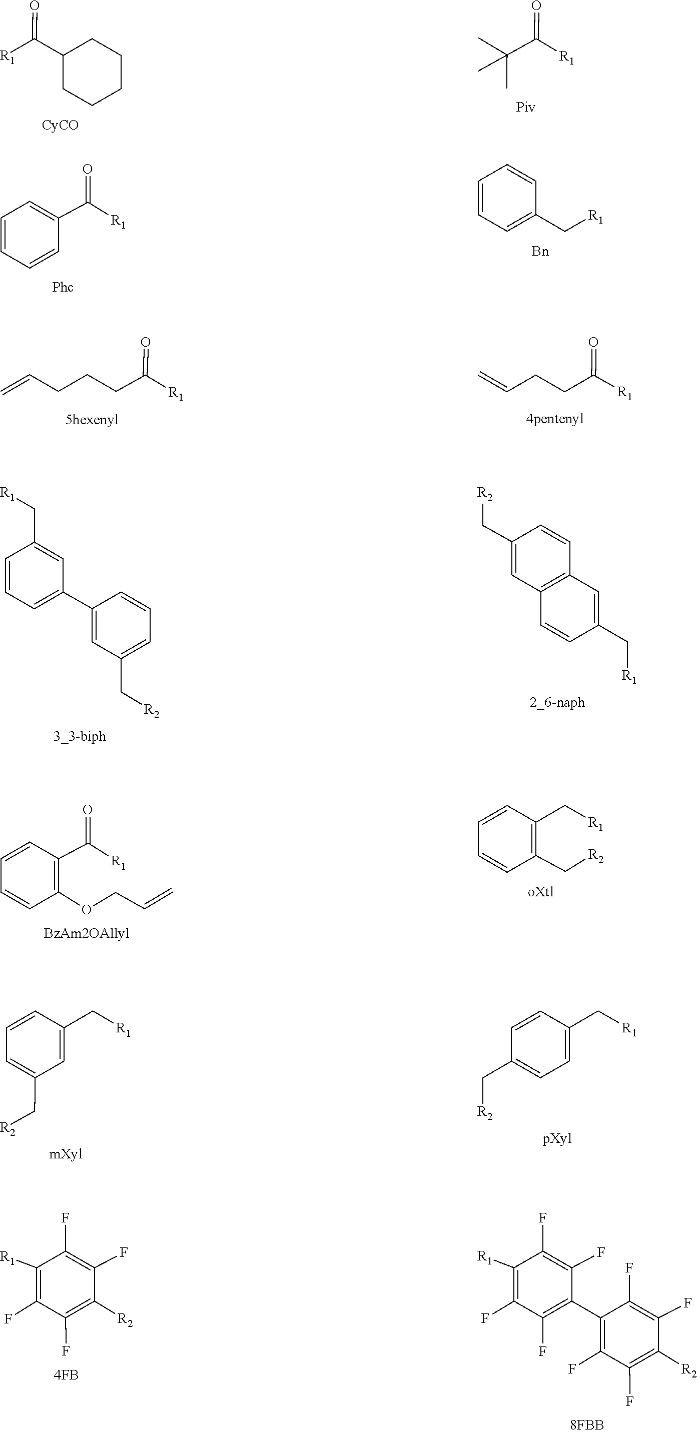

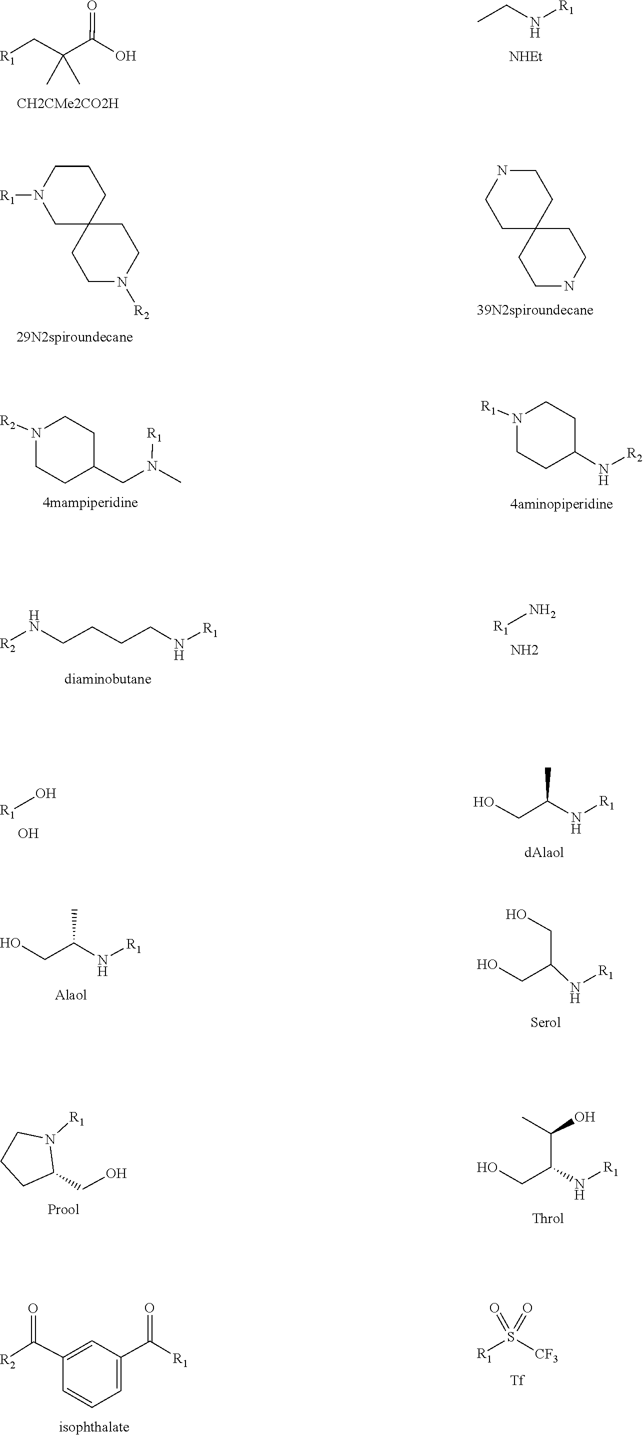

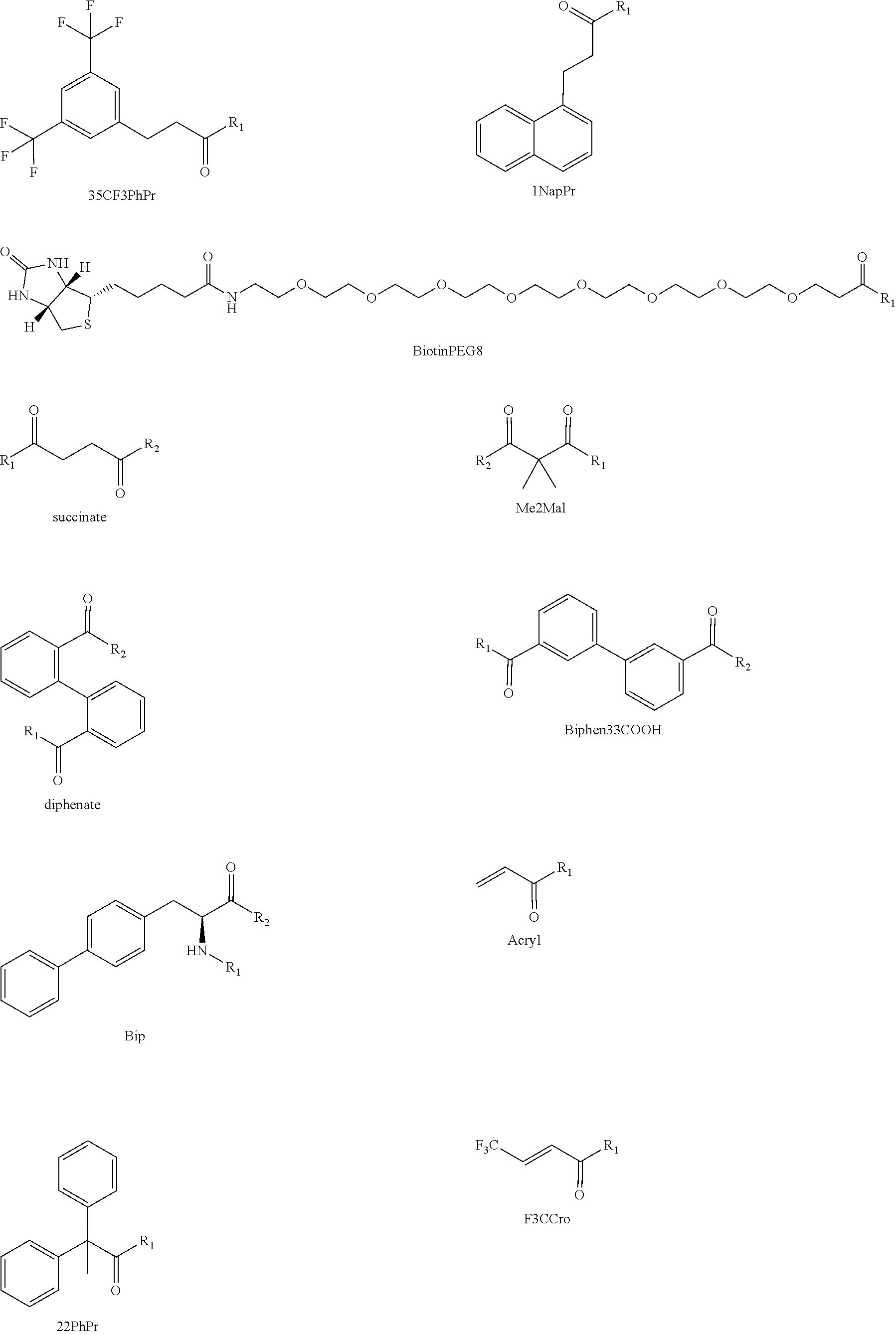

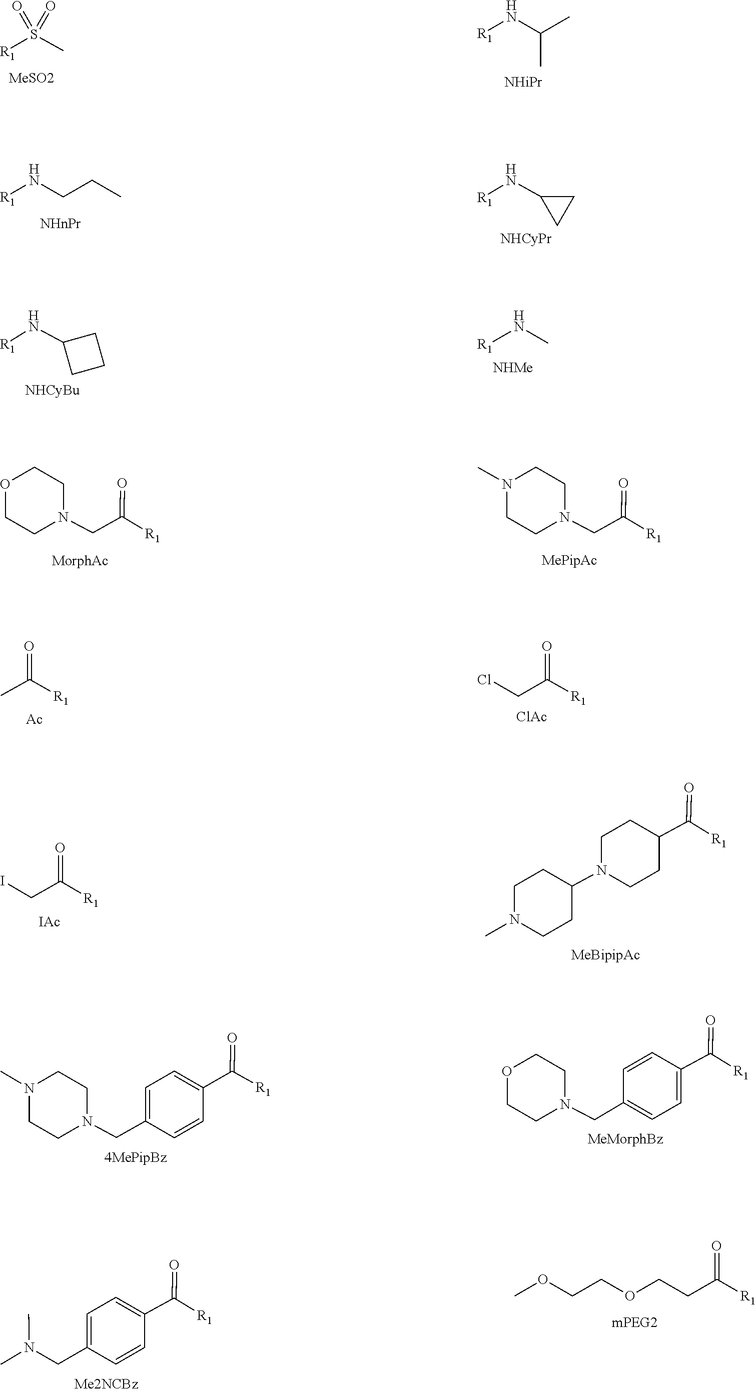

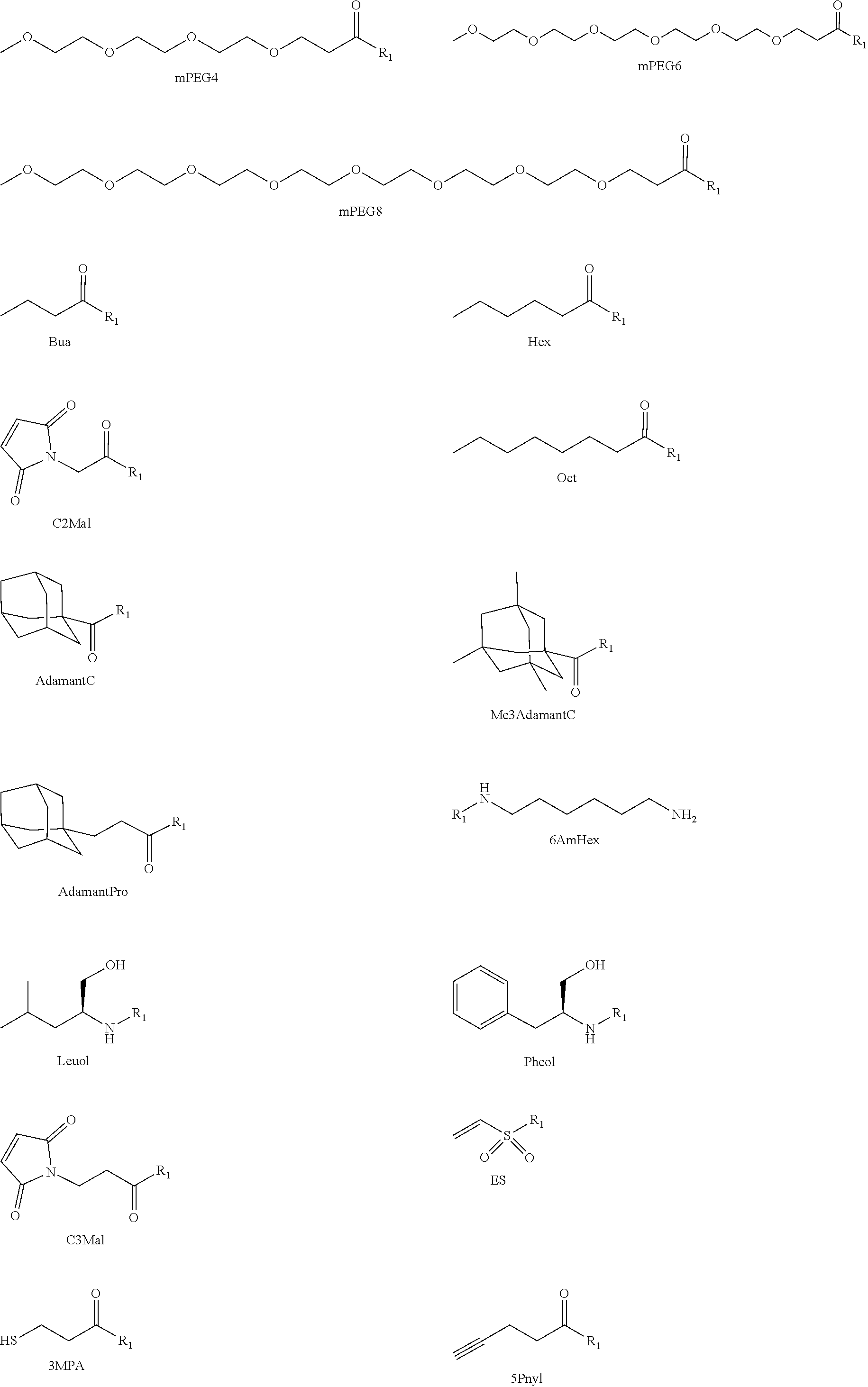

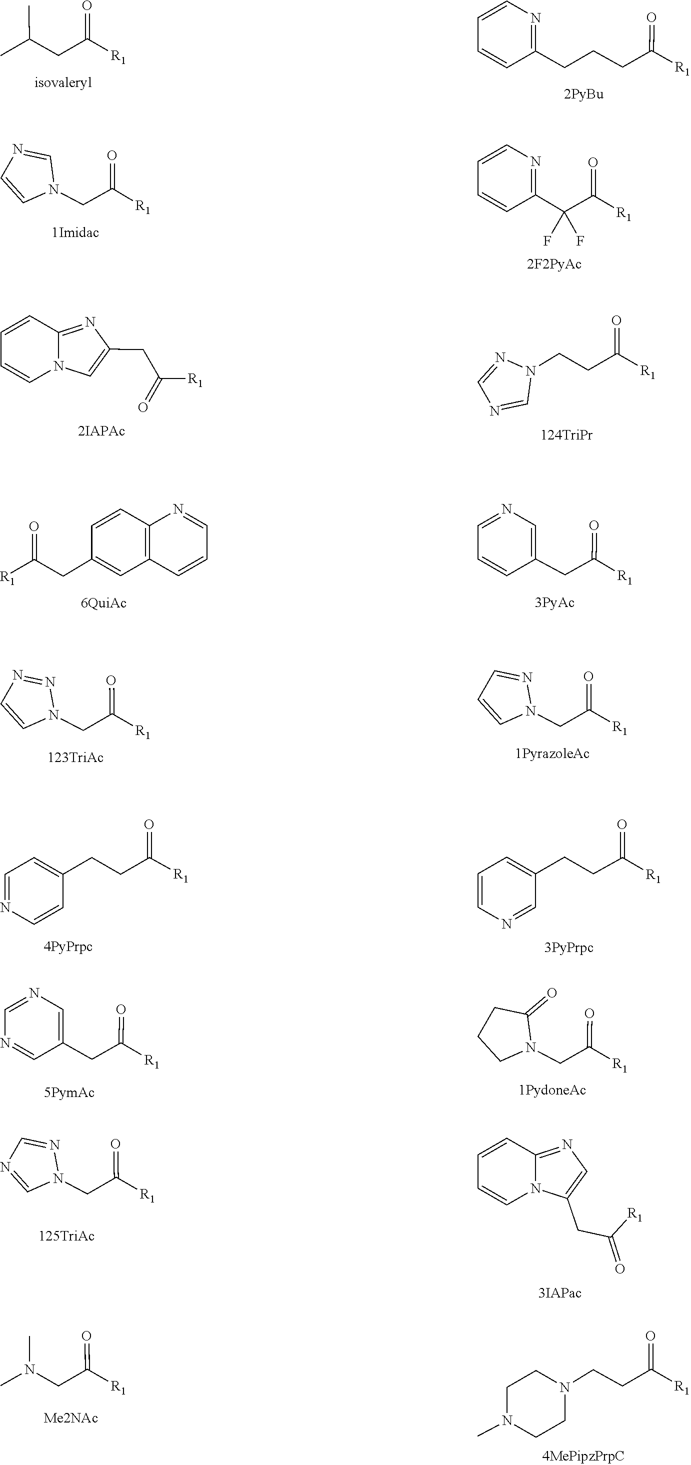

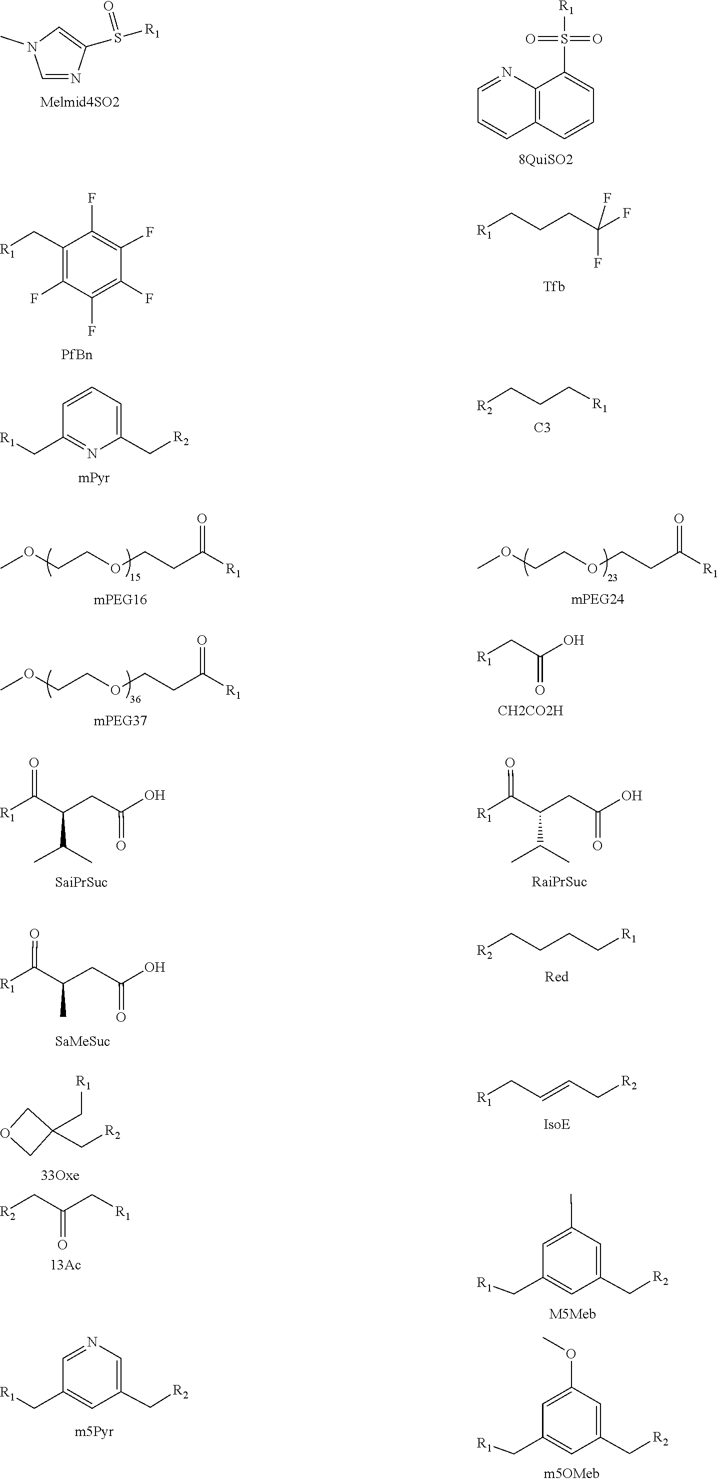

Certain moieties (e.g., moieties utilized in [ ] in various agents, amino acid residues, etc.)

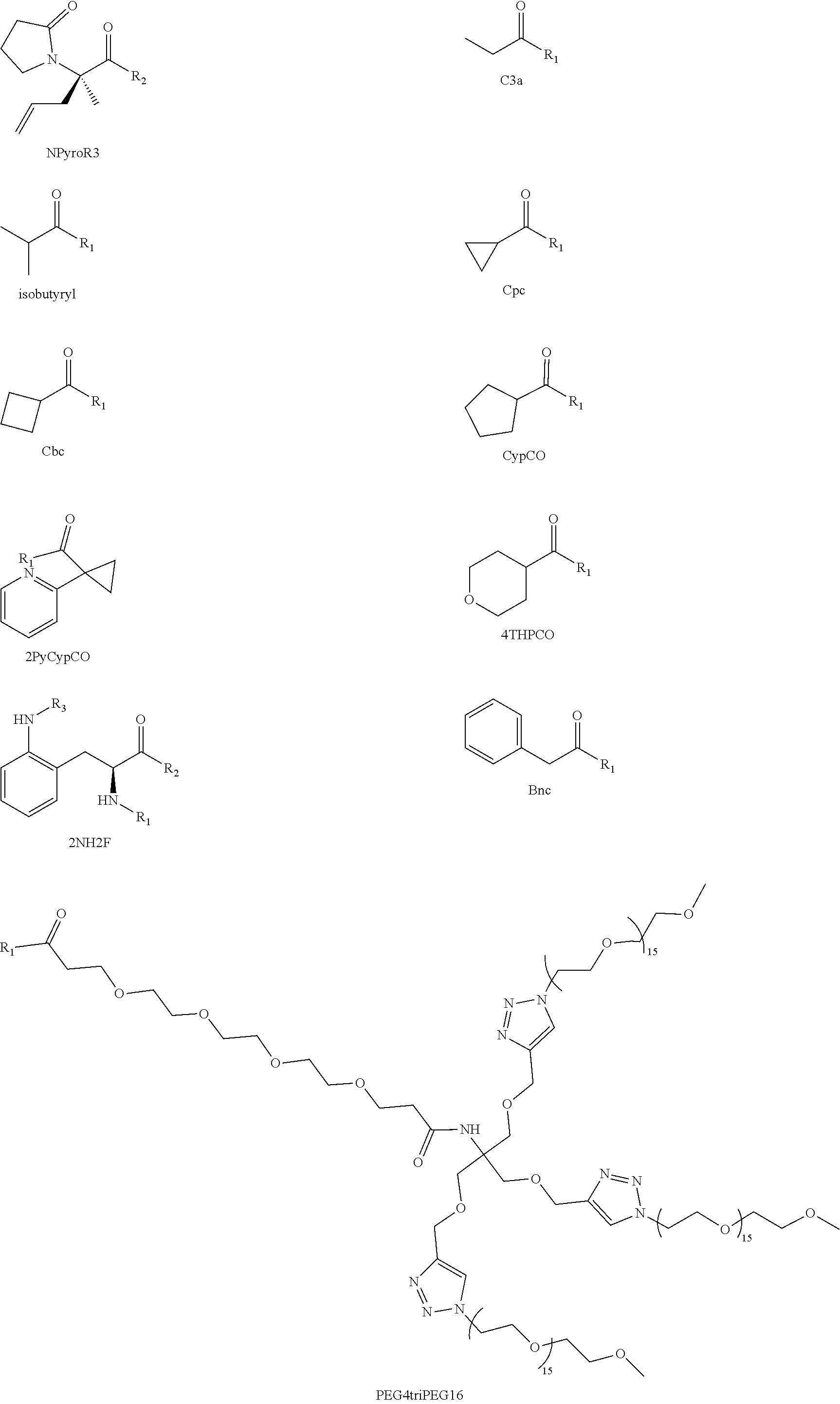

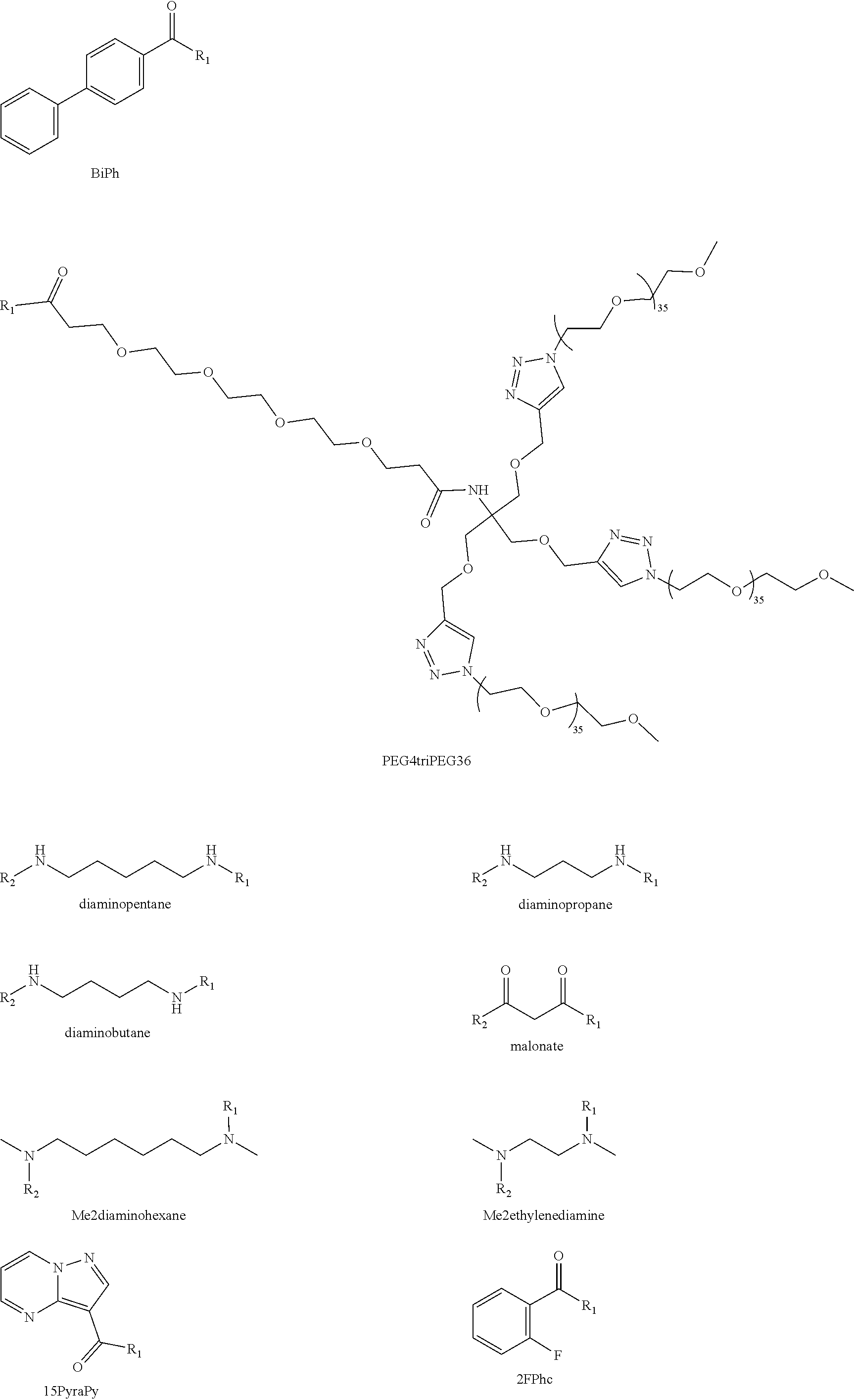

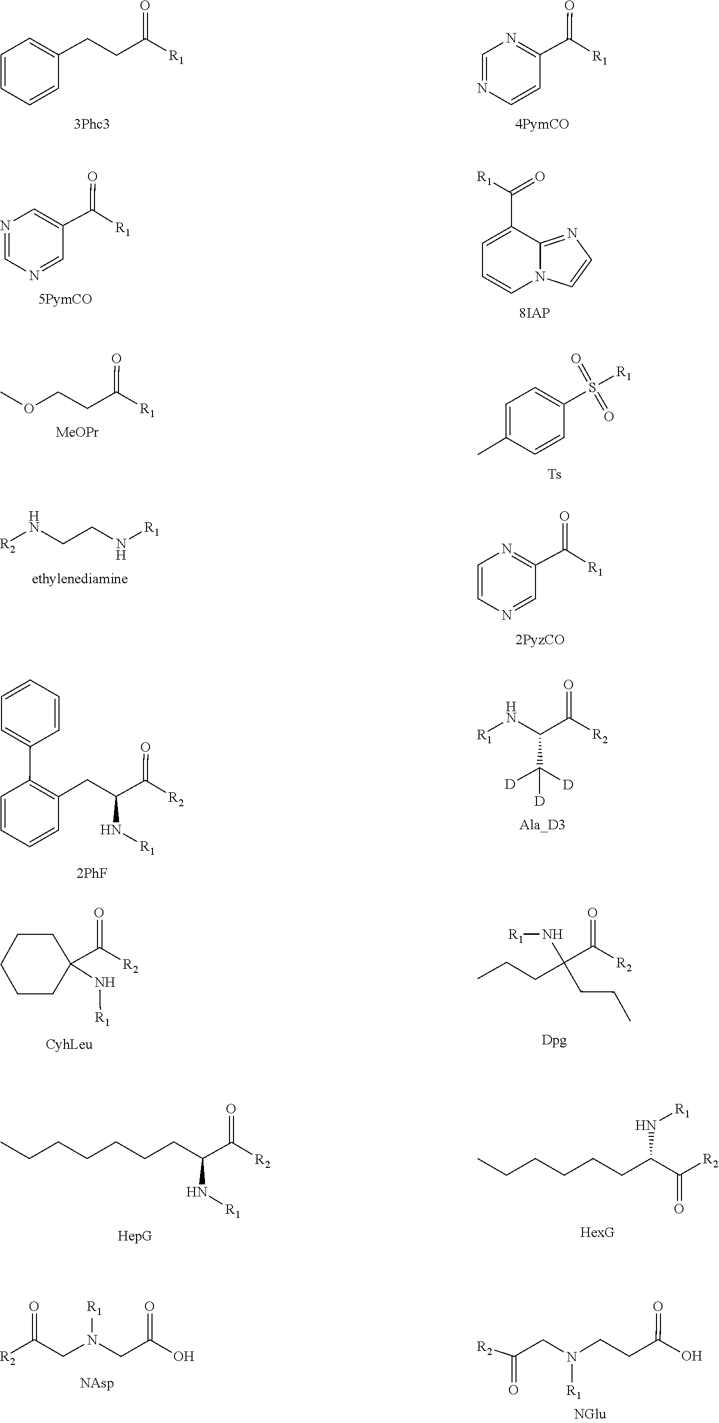

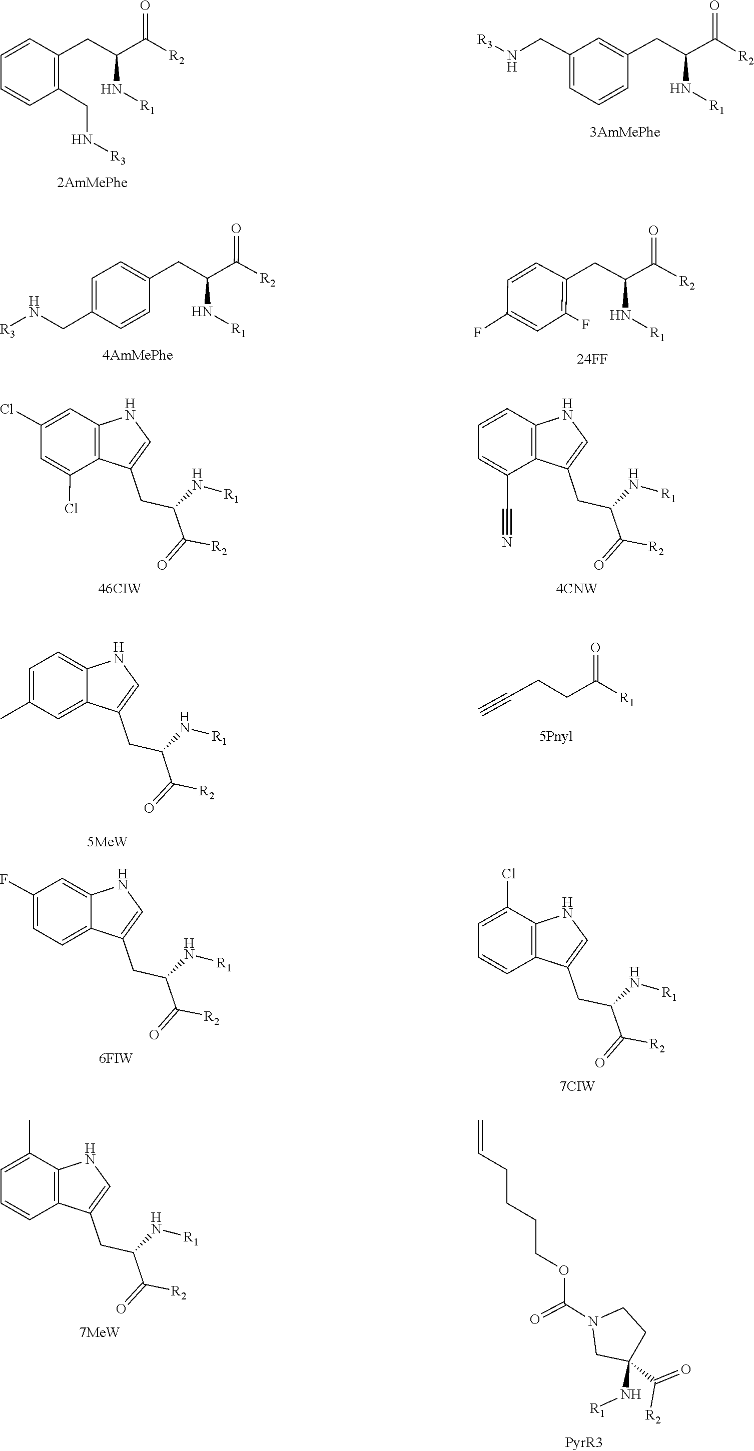

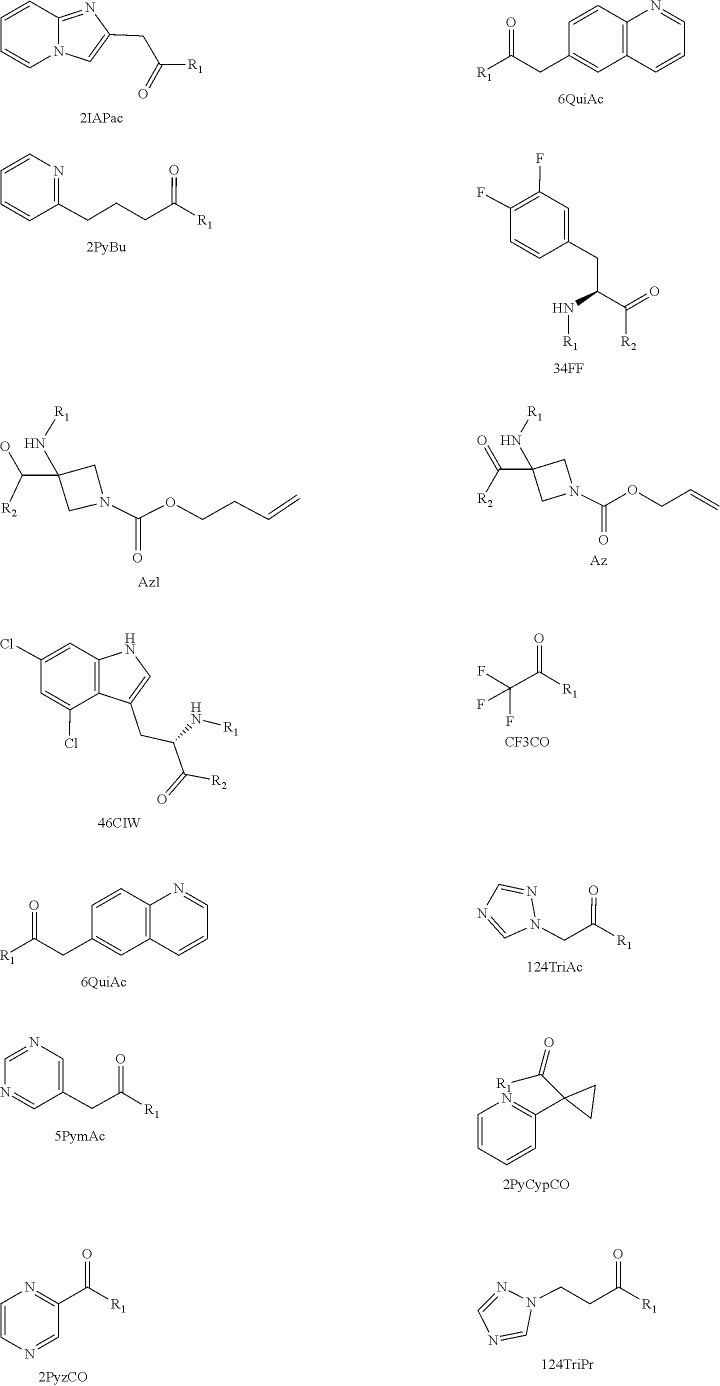

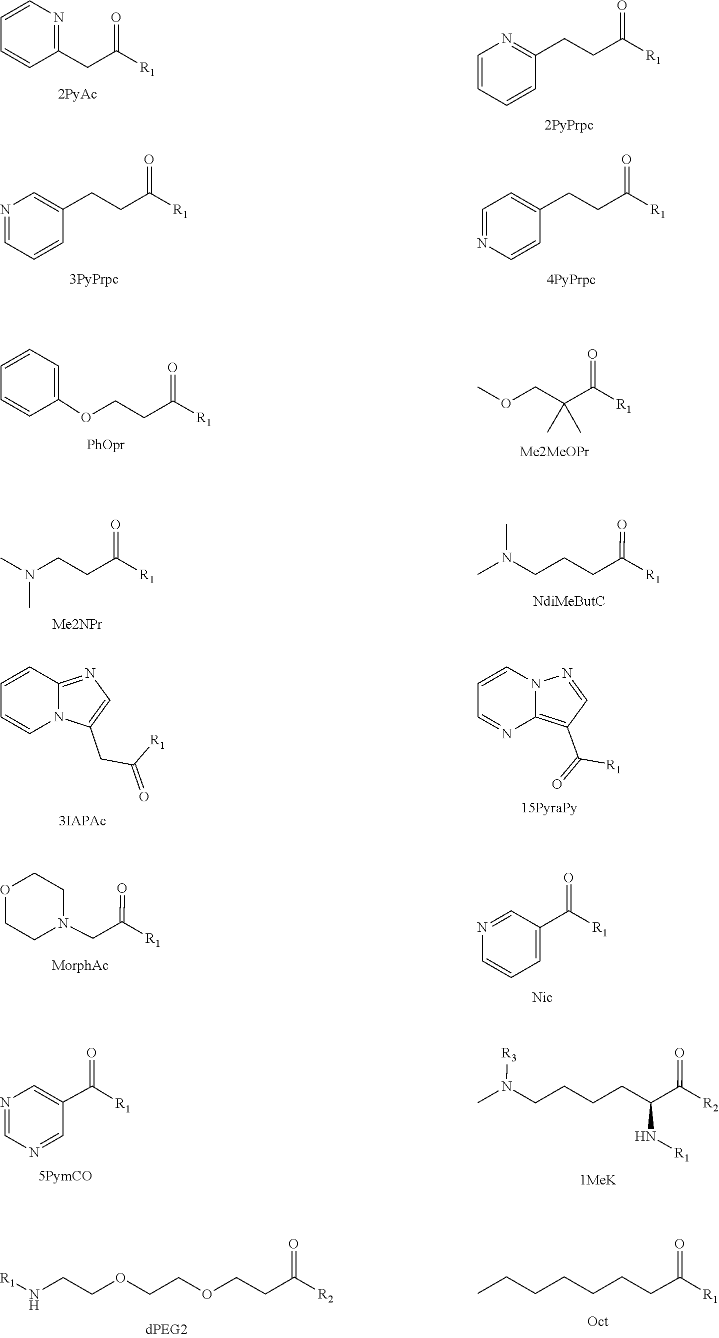

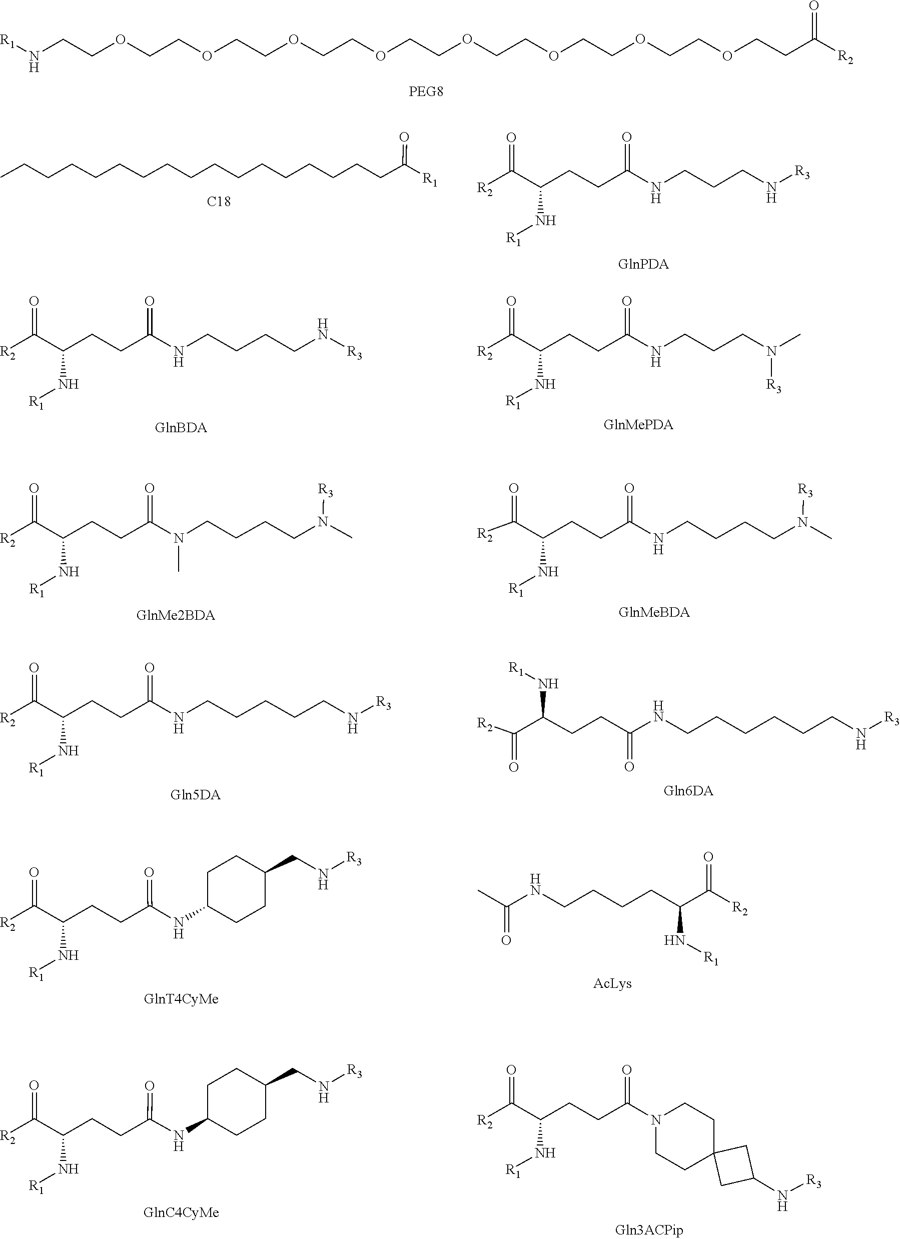

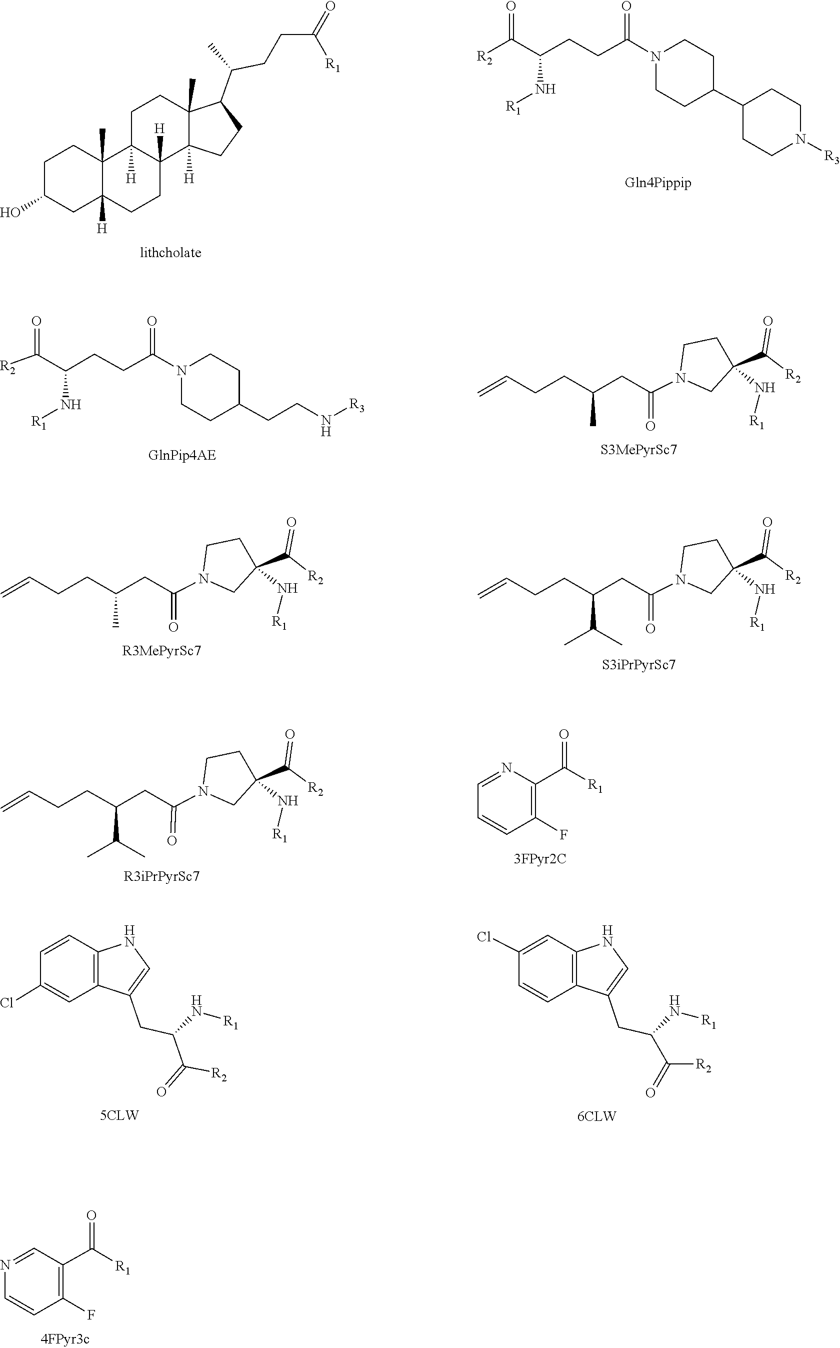

In some embodiments, within a bracket there are two moieties, e.g., [Ac-dPEG2], typically R₁ of the first is connected to R₁ of the latter. For example, in [Ac-dPEG2], R₁ of Ac is connected to R₁ of dPEG2. R₂ of dPEG2 can be connected to other moieties, e.g., in [Ac-dPEG2]-Lys, R₃ of Lys.

In some embodiments, the present disclosure provides an agent, e.g., a peptide agent (in various embodiments, a stapled peptide agent), comprising a moiety selected from the table above. In some embodiments, a residue is stapled, e.g., forming a staple with another moiety. In some embodiments, an agent comprises a staple formed between two moieties each independently selected from the table above. In some embodiments, a staple comprises a double bond. In some embodiments, a staple comprises an E double bond. In some embodiments, a staple comprises a Z double bond. In some embodiments, a double bond is converted into another moiety, e.g., to a saturated bond through hydrogenation, an epoxide through epoxidation, etc. In some embodiments, a moiety, e.g., an amino acid residue, comprises two groups that can be utilized for stapling. In some embodiments, an amino acid residue comprises two groups for stapling, e.g., B3, B4, B5, B6, Dap7Gly, Dap7Pent, DapAc7EDA, DapAc7PDA, Dap7Abu, etc. In some embodiments, a N-terminal group, e.g., 4pentenyl, 5hexenyl, etc., may be considered as part of the first amino acid residue for stapling. In some embodiments, amino acid residues with N-terminal groups (e.g., 4pentenyl, 5hexenyl, etc.) such as 4pentenyl-PL3, 5hexenyl-PL3, etc., comprise two groups, e.g., two double bonds, for stapling. In some embodiments, a group for stapling is a double bond. In some embodiments, each group for stapling is independently a double bond. In some embodiments, a group for stapling is a double bond and the other is not (e.g., amino group, or a group which is or comprises R₃). In some embodiments, an agent comprise two or more residues each independently comprising two or more groups (e.g., double bond) for stapling (e.g., 5hexenyl-PL3-Asp-AllylGly-B5-Asp-3COOHF-Ala-Ala-Phe-Leu-PyrS2-2F3MeF-BztA-Gln-NH2 or salt thereof (ESP-1), 4pentenyl-PL3-Asp-AllylGly-B5-Asp-3COOHF-Ala-Ala-Phe-Leu-PyrS2-2F3MeF-BztA-Gln-NH2 or salt thereof (ESP-2), etc., for stapling). In some embodiments, an agent comprises two or more amino acid residues each of which is independently bonded to two staples (e.g., 5hexenyl-PL3-Asp-AllylGly-B5-Asp-3COOHF-Ala-Ala-Phe-Leu-PyrS2-2F3MeF-BztA-Gln-NH2 (ESP-1) or salt thereof, 4pentenyl-PL3-Asp-AllylGly-B5-Asp-3COOHF-Ala-Ala-Phe-Leu-PyrS2-2F3MeF-BztA-Gln-NH2 or salt thereof (ESP2), etc. wherein the double bonds are utilized to form staples; in some embodiments, staples are formed through olefin metathesis; in some embodiments, double bonds in staples are further converted, e.g., into saturated bonds (e.g., through hydrogenation)). In some embodiments, agents, e.g., ESP-1, ESP-2, etc., comprise two or more staples within a short sequence and provide high stapling density, for example, a (i, i+2) and a (i, i+3) staple bonded to the same amino acid residue. In some embodiments, staples in provided agents are more evenly distributed out so that for any amino acid residues bonded to two or more staples, one and only one is (i, i+2) or (i, i+3). Thus, in some embodiments, an agent is not ESP-1 or ESP-2 (wherein ESP-1 and ESP-2 are not stapled, stapled, or modified post-stapling (e.g., hydrogenation to convert double bonds in staples to single bonds)). In some embodiments, an agent comprise one and no more than one residue comprising two or more residues for stapling. In some embodiments, an agent comprising one and no more than one amino acid residue that is bonded to two staples. In some embodiments, agents comprise staples having different types of structures and/or formed by different types of transformations. For example, in some embodiments, an agent comprises a staple whose formation does not comprises an olefin metathesis transformation and/or modification of a carbon-carbon double bond (e.g., hydrogenation). In some embodiments, such agents may provide improved properties, activities, design flexibility, manufacturing efficiency, etc.

In some embodiments, a compound has a structure selected from the table above, wherein R₁ is —OH. In some embodiments, a compound has a structure selected from the table above, wherein R₁ is —H. In some embodiments, a compound is a compound has the structure selected from the table above, wherein R₁ is —H or amino protecting group (e.g., Fmoc, tBoc, etc.) and R₂ is —OH, a carboxyl protecting or activating group, or a salt thereof. In some embodiments, a compound is a compound has the structure selected from the table above, wherein R₁ is —H or amino protecting group and R₂ is —OH, or a salt thereof. In some embodiments, a compound is a compound has the structure selected from the table above, wherein R₁ is —H and R₂ is —OH, or a salt thereof. In some embodiments, a compound is a compound has the structure selected from the table above, wherein R₁ is —H, R₂ is —OH and R₃ is —H, or a salt thereof. In some embodiments, R₃ is —H or a protecting group. In some embodiments, R₃ is —H. In some embodiments, a compound has a structure selected from the table above, wherein R₁ is an amino protection group, e.g., Fmoc, tBoc, etc. In some embodiments, a compound has a structure selected from the table above, wherein R₁ is an amino protecting group, e.g., Fmoc, tBoc, etc., and R₂ is —OH, or —COR₂ is an optionally substituted, protected or activated carboxyl group. In some embodiments, R₂ is —OH. In some embodiments, an amino acid residue has a structure selected from the table above, wherein each of R₁ and R₂ independently represents a connection site (e.g., for structure

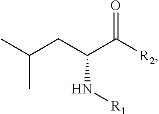

the residue is of the structure

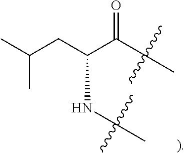 [SMILES: *N[C@H](CC(C)C)C(*)=O]

In some embodiments, an agent, a peptide or a stapled peptide comprises such an amino acid residue.

In some embodiments, a peptide comprises one or more residues of amino acids selected from the Table above. In some embodiments, a peptide comprises one or more residues of TfeGA. In some embodiments, a peptide comprises one or more residues of 2COOHF. In some embodiments, a peptide comprises one or more residues of 3COOHF.

Among other things, the present disclosure provides peptides, including stapled peptides, comprising residues of amino acids described herein. In some embodiments, the present disclosure provides various methods comprising utilizing amino acids, optionally protected and/or activated, as described herein. In some embodiments, the present disclosure provides methods for preparing peptides, comprising utilizing amino acids, typically protected and/or activated, as described herein. For example, in some embodiments, various amino groups are Fmoc protected for peptide synthesis (particularly for forming backbone peptide bonds). In some embodiments, various side chain carboxylic acid groups are t-Bu protected (—C(O)—O-tBu).

In some embodiments, the present disclosure provides methods, comprising replacing one or more acidic amino acid residues, e.g., Asp, Glu, etc., in a first compound, each independently with a provided amino acid residue, e.g., TfeGA, 2COOHF, 3COOHF, etc., to provide a second compound. In some embodiments, each of the first and second compounds is independently or independently comprises a peptide. In some embodiments, a second compound provides improved properties and/or activities (e.g., lipophilicity, LogD, etc.) compared to a first compound. In some embodiments, a second compound provides, in addition to improved properties such as lipophilicity, one or more comparable or improved other properties and/or activities (e.g., solubility and/or target binding) compared to a first compound.

In some embodiments, an agent, e.g., a peptide, a stapled peptide, a stitched peptide, etc., is less than about 5000 Daltons in mass. In some embodiments, an agent is greater than or equal to about 900 Daltons and less than about 5000 Daltons in mass. In some embodiments, an agent is greater than or equal to about 1500 Daltons and less than about 5000 Daltons in mass. In some embodiments, an agent is greater than or equal to about 2000 Daltons and less than about 5000 Daltons in mass. In some embodiments, an agent is greater than or equal to about 2500 Daltons and less than about 5000 Daltons in mass. In some embodiments, an agent is greater than or equal to about 1000 Daltons and less than about 3000 Daltons in mass. In some embodiments, an agent is greater than or equal to about 1500 Daltons and less than about 3000 Daltons in mass. In some embodiments, an agent is greater than or equal to about 1500 Daltons and less than about 2500 Daltons in mass. In some embodiments, an agent is greater than or equal to about 1600 Daltons and less than about 2200 Daltons in mass. In some embodiments, the agent is no more than about 900 Daltons in mass. In some embodiments, an agent is no more than about 500 Daltons in mass. In some embodiments, an agent is no more than about 300 Daltons in mass. In some embodiments, an agent is no more than about 200 Daltons in mass.

### Characterization

In some embodiments, agents, e.g., peptides, are characterized with respect to, for example, one or more characteristics such as binding characteristics—e.g., with respect to a particular target of interest (e.g., beta-catenin or a portion thereof), stability characteristics, for example in solution or in dried form, cell permeability characteristics, solubility, lipophilicity, etc.

In some embodiments, a binding characteristic may be or comprise specificity, affinity, on-rate, off-rate, etc, optionally under (or over a range of) specified conditions such as, for example, concentration, temperature, pH, cell type, presence or level of a particular competitor, etc.

As will be appreciated by those skilled in the art, assessments of characteristics as described herein may involve comparison with an appropriate reference (e.g., a positive or negative control) which may, in some embodiments, be a contemporaneous reference or, in some embodiments, a historical reference.

In some embodiments, desirable characteristics may be, for example: binding to a desired target (e.g., a dissociation constant (K_{D}) of at least less than about 1 μM, and preferably a K_{D} of less than about 50 nM); cell penetration (e.g., as measured by fluorescence-based assays or mass spectrometry of cellular fractions, etc.); solubility (e.g., soluble at less than about 1000 uM agent, or soluble at less than about 500 uM agent, or soluble at less than about 100 uM agent, or less than about 50 uM, or less than about 35 uM); activity (e.g., modulating one or more functions of a target, which may be assessed in a cellular reporter assay (e.g., with an IC50 of less than a concentration, e.g., less than about 1 μM, less than about 500 nM, less than about 50 nM, less than about 10 nM, etc.), an animal model (e.g., various animal models for conditions, disorders or diseases, e.g., mouse melanoma models Braf^{V600E}/Pten^{-/-} and Braf^{V600E}/Pten^{-/-}/CAT-STA) and/or a subject; stability, which may be assessed using a number of assays (e.g., in a rat pharmacokinetic study (e.g., administered via oral, iv, ip, etc.) with a terminal half-life of greater than a suitable time, e.g., 1 hour); low toxicity, which might be assessed by a number of assays (e.g., a standard ADME/toxicity assays); and/or low levels of cytotoxicity (e.g., low levels of lactate dehydrogenase (LDH) released from cells when treated at a suitable concentration, e.g., about 10 μM of a peptide). In some embodiments, an agent of the invention comprises an affinity of less than about 10 nM, for example, an IC50 of 7 nM).

In some embodiments, provided agents can bind to targets, e.g., beta-catenin, with an EC 50 of no more than about 2000 nM. In some embodiments, an EC50 is no more than about 1500 nM. In some embodiments, an EC50 is no more than about 1000 nM. In some embodiments, an EC50 is no more than about 500 nM. In some embodiments, an EC50 is no more than about 300 nM. In some embodiments, an EC50 is no more than about 200 nM. In some embodiments, an EC50 is no more than about 100 nM. In some embodiments, an EC50 is no more than about 75 nM. In some embodiments, an EC50 is no more than about 50 nM. In some embodiments, an EC50 is no more than about 25 nM. In some embodiments, an EC50 is no more than about 10 nM. In some embodiments, an EC50 is no more than about 5 nM. In some embodiments, an EC50 is measured by fluorescence polarization as described in the Examples.

In some embodiments, the present disclosure provides agents, e.g., stapled peptides, with suitable solubility for various purposes. In some embodiments, solubility of provided agents, e.g., in PBS, is about or at least about 5-100 uM (e.g., about or at least about 5, 10, 15, 20, 25, 30, 40, 50, 60, 70, 80, 90, or 100 uM). In some embodiments, solubility is about or at least about 25 uM. In some embodiments, solubility is about or at least about 30 uM. In some embodiments, solubility is about or at least about 40 uM. In some embodiments, solubility is about or at least about 50 uM. In some embodiments, provided agents, e.g., stapled peptides, are protein bound in serum; in some embodiments, they are at least about 85%, 90%, or 95% protein bound in serum. In some embodiments, provided agents are over 95% protein bound in serum.

In some embodiments, provided agents can traverse a cell membrane of an animal cell. In some embodiments, provided agents can traverse a cell membrane of a human cell.

Among other things, provided agents can bind to motifs, residues, or polypeptides. In some embodiments, provided agents bind to beta-catenin. In some embodiments, a dissociation constant (K_{D}) is about 1 nM to about 1 uM. In some embodiments, a K_{D} is no more than about 1 uM. In some embodiments, a K_{D} is no more than about 500 nM. In some embodiments, a K_{D} is no more than about 250 nM. In some embodiments, a K_{D} is no more than about 100 nM. In some embodiments, a K_{D} is no more than about 50 nM. In some embodiments, a K_{D} is no more than about 25 nM. In some embodiments, a K_{D} is no more than about 10 nM. In some embodiments, a K_{D} is no more than about 5 nM. In some embodiments, a K_{D} is no more than about 1 nM. As appreciated by those skilled in the art, various technologies are available and can be utilized to measure K_{D} in accordance with the present disclosure. In some embodiments, K_{D} is measured by Surface Plasmon Resonance (SPR) as illustrated herein.

In some embodiments, provided agents binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

In some embodiments, provided agents have one or more or all of the following interactions with beta-catenin:

Direct interactions (), water mediated [], non-
polar contacts {}
(SEQ ID NO: 3)
LQIL{AY}(G){NQ}ES(K)LIILA (residue 301-317 of
Uniprot P35222 sequence)
(SEQ ID NO: 4)
SRVL{(K)V}LS{V}CSSN (residue 341-353 of Uniprot
P35222 sequence)
(SEQ ID NO: 5)
RLV{QN}C{L}(W)TL{R}(N)LSDA (residue 376-391 of
Uniprot P35222 sequence)
(SEQ ID NO: 6)
LGSD[D]I(N){V}V{TC}AAGI (residue 409-423 of
Uniprot P35222 sequence)

In some embodiments, an agent, e.g., a peptide, binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, K345, V346, V349, Q379, N380, L382, W383, R386, N387, D413, N415, V416, T418, and C419. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, R386, N387, D413, and N415. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, N387, D413, and N415.

In some embodiments, provided agents interact with beta-catenin at one or more (e.g., 1, 2, 3, 4, 5, 6, 7, 8 or 9) of G307, K312, K345, Q379, L382, W383, N387, N415 and V416. In some embodiments, provided agents interact with beta-catenin at one or more (e.g., 1, 2, 3, 4, 5, 6, 7, 8, 9 or 10) of Y306, G307, K312, K345, Q379, L382, W383, N387, N415 and V416. In some embodiments, provided agents interact with beta-catenin at one or more (e.g., 1, 2, 3, 4, 5, 6, 7, 8, 9 or 10) of G307, K312, K345, Q379, L382, W383, R386, N387, N415 and V416. In some embodiments, provided agents interact with beta-catenin at one or more (e.g., 1, 2, 3, 4, 5, 6, 7, 8, 9, 10 or 11) of Y306, G307, K312, K345, Q379, L382, W383, R386, N387, N415 and V416. In some embodiments, provided agents interact with beta-catenin at one or more (e.g., 1, 2, 3, 4, 5, 6, 7, 8, 9, 10, 11 or 12) of Y306, G307, K312, K345, V349, Q379, L382, W383, R386, N387, N415 and V416. In some embodiments, provided agents interact with beta-catenin at one or more (e.g., 1, 2, 3, 4, 5, 6, or 7) of G307, K312, K345, W383, R386, N387, D413 and N415. In some embodiments, provided agents interact with beta-catenin at one or more (e.g., 1, 2, 3, 4, 5, 6, or 7) of G307, K312, K345, W383, N387, D413 and N415. In some embodiments, provided agents interact with beta-catenin at one or both of K312 and R386. In some embodiments, provided agents interact with G307. In some embodiments, provided agents interact with K312. In some embodiments, provided agents interact with beta-catenin at one or more of K345, W383, D413 and N415. In some embodiments, provided agents interact with beta-catenin at one or more of K345 and W383. In some embodiments, provided agents interact with beta-catenin at one or more of D413 and N415. In some embodiments, provided agents interact with Y306. In some embodiments, provided agents interact with G307. In some embodiments, provided agents interact with K312. In some embodiments, provided agents interact with K345. In some embodiments, provided agents interact with V349. In some embodiments, provided agents interact with Q379. In some embodiments, provided agents interact with L382. In some embodiments, provided agents interact with W383. In some embodiments, provided agents interact with R386. In some embodiments, provided agents interact with N387. In some embodiments, provided agents interact with D413. In some embodiments, provided agents interact with N415. In some embodiments, provided agents interact with V416.

In some embodiments, provided agents interact with one or more of amino acid residues that are or correspond to K312, R386, K345 and W383 of SEQ ID NO: 1. In some embodiments, provided agents interact with one or more of amino acid residues that are or correspond to K312 and R386 of SEQ ID NO: 1. In some embodiments, interaction with an amino acid residue can be assessed through mutation of such an amino acid residue (e.g., mutation of K, R, etc. to D, E, etc.).

As those skilled in the art reading the present disclosure will appreciate, in some embodiments, interactions with beta-catenin may be assessed by contacting an agent with either a full-length or a portion of beta-catenin. In some embodiments, a portion of beta-catenin comprises the interacting residues above. In some embodiments, a portion of beta-catenin is or comprises SEQ ID NO: 2. In some embodiments, a portion of beta-catenin is expressed with a tag (e.g., for purification, detection, etc.). In some embodiments, a tag is a fluorescent tag. In some embodiments, a tag is for detection. In some embodiments, a tag is for purification and detection. In some embodiments, a tag is a purification tag. In some embodiments, a tag is or comprises biotin. Many other types of tags are available in the art and can be utilized in accordance with the present disclosure.

Various technologies can be utilized for characterizing and/or assessing provided technologies (e.g., agents (e.g., various peptides), compositions, methods, etc.) in accordance with the present disclosure. As described herein, in some embodiments, a useful technology is or comprises fluorescence polarization. In some embodiments, a useful technology assesses LogP or LogD. In some embodiments, a useful technology is or comprises a CHI LogD assay. In some embodiments, a useful technology assesses solubility. In some embodiments, a useful technology is or comprises NanoBRET. In some embodiments, a useful technology is or comprises a reporter assay (e.g., DLD1 reporter assay). In some embodiments, a useful technology is or comprises alphascreen. Certain useful protocols are described in the Examples. Those skilled in the art appreciate that suitable adjustments may be made to such protocols, e.g., according to specific conditions, agents, purposes, etc.

### Production

Various technologies are known in the art for producing provided agents. For example, various technologies for preparing small molecules, peptides (including stapled peptides) may be utilized in accordance with the present disclosure. Those skilled in the art, reading the present disclosure will well appreciate which such technologies are applicable in which aspects of the present disclosure in accordance with the present disclosure.

Stapling may be performed during and/or after peptide chain synthesis. In some embodiments, the present disclosure provides an unstapled peptide agent whose sequence is one described in Table E2 or Table E3. In some embodiments, amino acid residues are optionally protected for peptide synthesis (e.g., peptide synthesis using Fmoc-protected amino acids wherein certain side chains may be protected). In some embodiments, one or more stapling are achieved through olefin metathesis. In some embodiments, two or more stapling are formed through one olefin metathesis process. In some embodiments, the present disclosure provides a stapled peptide agent described in Table E2 or Table E3 or a salt thereof (e.g., a pharmaceutically acceptable salt thereof). In some embodiments, the present disclosure provides a stereoisomer of a stapled peptide agent described in Table E2 or Table E3 or a salt thereof (e.g., a pharmaceutically acceptable salt thereof). In some embodiments, the present disclosure provides a E/Z stereoisomer of a stapled peptide agent described in Table E2 or Table E3 or a salt thereof (e.g., a pharmaceutically acceptable salt thereof). In some embodiments, from the N to C direction, an olefin double bond in the first staple that comprising such a bond is Z, and an olefin double in the second staple that comprising such a bond is E (Z-E); in some embodiments, it is (Z-Z); in some embodiments, it is (E-Z); in some embodiments, it is (E-E). In some embodiments, from the N to C direction, an olefin double bond in the first (i, i+2), (i, i+3) or (i, i+4) staple that comprising such a bond is Z, and an olefin double in the first (i, i+7) staple that comprising such a bond is E (Z-E); in some embodiments, it is (Z-Z); in some embodiments, it is (E-Z); in some embodiments, it is (E-E). In some embodiments, an agent comprises an olefin double bond in a third staple, and it is E; in some embodiments, it is Z. In some embodiments, an agent comprises an olefin double bond in a fourth staple, and it is E; in some embodiments, it is Z.

In some embodiments, one or more or all staples are formed after chain extension. In some embodiments, one or more or all staples are formed during chain extension. In some embodiments, one or more or all staples by metathesis are formed after chain extension. In some embodiments, one or more or all staples by metathesis are formed during chain extension.

In some embodiments, the present disclosure provides a method, comprising
a) preparing a first compound comprising two moieties each of which independently comprises an olefin double bond;
b) providing a second compound by stapling the two moieties by olefin metathesis of an olefin double bond of one moiety with an olefin double bond of the other to form a first-formed staple;
c) add one or more additional moieties to the second compound to provide a third compound which comprising two moieties each of which independently comprises an olefin double bond; and
d) providing a fourth compound by stapling the two moieties in the third compound by olefin metathesis of an olefin double bond of one moiety with an olefin double bond of the other to form a second-formed staple.

In some embodiments, a moiety is an amino acid residue. In some embodiments, each moiety is independently an amino acid residue. In some embodiments, each moiety is independently an amino acid residue comprising a terminal olefin as described herein. In some embodiments, there are two olefin double bonds in one moiety, e.g., of the first compound. For example, in some embodiments, such a moiety is B5. In some embodiments, two moieties of a first compound is independently X⁴ and X¹¹. In some embodiments, a first-formed staple is a (i, i+7) staple. In some embodiments, a first compound comprises —X⁴X⁵X⁶X⁷X⁸X⁹X¹⁰X¹¹—. In some embodiments, a first compound comprises —X⁴X⁵X⁶X⁷X⁸X⁹X¹⁰X¹¹X¹²X¹³X¹⁴—. In some embodiments, a first compound comprises a staple. In some embodiments, a staple is a (i, i+4) staple. In some embodiments, a staple is between X¹⁰ and X¹⁴. In some embodiments, an olefin double bond in a third compound is present in the first compound (e.g., an unstapled olefin double bond of B5). In some embodiments, one and only one amino acid residue comprises an olefin double bond is added to the second compound. In some embodiments, ae third compound is or comprises —X¹X²X³X⁴X⁵X⁶X⁷X⁸X⁹X¹⁰X¹¹—. In some embodiments, a third compound is or comprises —X¹X²X³X⁴X⁵X⁶X⁷X⁸X⁹X¹⁰X¹¹X¹²X¹³X¹⁴—. In some embodiments, a first- and second-formed staples are bonded to the same amino acid residue. In some embodiments, a first- and second-formed staples are bonded to the same atom. In some embodiments, a second-formed staple is a (i, i+2), (i, i+3) or (i, i+4) staple. In some embodiments, two moieties in the third compound is independently X¹ and X⁴. In some embodiments, a first-formed staple is formed with E selectivity as described herein (e.g. about 1.1:1, 1.2:1, 1.3:1, 1.4:1, 1.5:1, 2:1, 3:1, 4:1, 5:1, 10:1, 20:1, 30:1, 40:1, 50:1, or more). In some embodiments, a second-formed staple is formed with Z selectivity as described herein (e.g., about 1.1:1, 1.2:1, 1.3:1, 1.4:1, 1.5:1, 2:1, 3:1, 4:1, 5:1, 10:1, 20:1, 30:1, 40:1, 50:1, or more). In some embodiments, synthesis may be performed on a solid support (e.g., solid phase peptide synthesis), and a compound or an agent may be on a solid support. In some embodiments, stapling during chain extension, or individually performed stapling for one or more staples, can provide advantages, e.g., increased selectivity, yield, purity, etc.

In some embodiments, two or more staples are formed in a metathesis reaction. In some embodiments, all staples formed by metathesis are formed in a metathesis reaction. In some embodiments, each of such staples are formed through olefin metathesis of terminal olefins. In some embodiments, multiple staples are formed after full lengths of peptides have been achieved. In some embodiments, one or more staples comprising double bonds are formed after full lengths of peptides have been achieved. In some embodiments, all staples comprising double bonds are formed after full lengths of peptides have been achieved. In some embodiments, one or more staples formed through metathesis are formed after full lengths of peptides have been achieved. In some embodiments, all staples formed through metathesis are formed after full lengths of peptides have been achieved.

In some embodiments, stepwise stapling, in which two or more staples are formed in two or more steps, were performed. In some embodiments, stepwise stapling provides improved levels of selectivity to form a desired product (e.g., I-66) over other compounds, e.g., stereoisomers (e.g., for I-66, I-67). In some embodiments, an improvement is about or at least about 1.5, 2, 2.5, 3, 4, 5, 6, 7, 8, 9, or 10 fold. In some embodiments, an improvement is assessed by comparing percentage of a desired product among all related stereoisomers. In some embodiments, an improvement is assessed by ratios of a desired product versus a stereoisomer (e.g., I-66 versus I-67). In some embodiments, two staples comprising olefin double bonds are formed in two separate steps. In some embodiments, two staples formed by metathesis are formed in two separate steps. In some embodiments, two staples bonded to the same amino acid residue are formed in two separate steps. In some embodiments, two staples bonded to the same atom are formed in two separate steps. In some embodiments, two staples bonded to the same carbon atom are formed in two separate steps. In some embodiments, two staples formed from B5 are formed in two separate steps. In some embodiments, a provided technologies comprise a third step forming a third staple. In some embodiments, each staple is formed in a separate step. In some embodiments, the present disclosure provides a method for preparing a stapled peptide, comprising:
1) reacting a first reactive group with a second reactive group to form a first staple, wherein the first and second reactive groups are in two different amino acid residues; and
2) reacting a third reactive group with a fourth reactive group to form a second staple, wherein the third and fourth reactive groups are in two different amino acid residues.

Alternatively or additionally, in some embodiments, a method comprises reacting a fifth reactive group with a sixth reactive group to form a third staple, wherein the fifth and sixth reactive groups are in two different amino acid residues. In some embodiments, a third staple is formed before a first and second staples.

In some embodiments, a first staple is formed through a metathesis reaction. In some embodiments, each of the first and second reactive groups independently is or comprises a double bond. In some embodiments, each of the first and second reactive groups is independently a terminal olefin. In some embodiments, a first staple is formed through olefin metathesis. In some embodiments, a first staple is an (i, i+7) staple. Various metathesis technologies may be utilized in accordance with the present disclosure to form a first staple. In some embodiments, a metathesis reaction is performed in the presence of a catalyst. In some embodiments, a catalyst is Hoveyda-Grubbs M720 catalyst (CAS 301224-40-8). In some embodiments, a first staple is between X⁴ and X¹¹.

In some embodiments, a second staple is formed through a metathesis reaction. In some embodiments, each of the third and fourth reactive groups independently is or comprises a double bond. In some embodiments, each of the third and fourth reactive groups is independently a terminal olefin. In some embodiments, a second staple is formed through olefin metathesis. In some embodiments, a second staple is an (i, i+3) staple. Various metathesis technologies may be utilized in accordance with the present disclosure to form a second staple. In some embodiments, a metathesis reaction is performed in the presence of a catalyst. In some embodiments, a catalyst is Grubbs M102 catalyst (CAS 172222-30-9). In some embodiments, a second staple is between X¹ and X⁴.

In some embodiments, one of the first and second reactive groups, and one of the third and fourth reactive groups, are in the same amino acid residues. In some embodiments, they are independently in a side chain and the two side chains are bonded to the same atom. In some embodiments, the two side chains are bonded to the same carbon atom, e.g., as in B5. In some embodiments, the first and second staples are bonded to the same amino acid residue. In some embodiments, they are bonded to same atom. In some embodiments, they are bonded to the same carbon, e.g., in B5.

In some embodiments, a third staple comprises an amide group, e.g., —C(O)N(R′)— wherein R′ is as described herein. In some embodiments, a third staple comprises —C(O)NH—. In some embodiments, a third staple is a (i, i+4) staple. In some embodiments, one of the fifth and the sixth reactive groups is or comprises an amino group or an activated form thereof, and the other is or comprises an acid group, e.g., a carboxyl group, or an activated form thereof. In some embodiments, a third staple is formed through an amidation reaction. In some embodiments, a third staple is not formed by a metathesis reaction. In some embodiments, a third staple does not comprise an olefin double bond. Various amidation technologies are available and may be utilized herein. As described herein, other types of staples may be utilized and constructed as well. See, for example, preparation of I-66, I-335, etc. in the Examples. In some embodiments, a third staple is between X¹⁰ and X¹⁴.

In some embodiments, as described herein, one or more stapling steps are independently performed before full lengths are achieved. For example, in some embodiments, a third staple is formed before the two amino acid residues comprising the first and second reactive groups are both installed. Alternatively or additionally, in some embodiments, a first staple is formed before the two amino acid residues comprising the third and fourth reactive groups are both installed. In some embodiments, a third staple is formed after an amino acid residue comprising one of the first and second reactive group is installed but before an amino acid residue comprising the other of the first and second reactive group is installed. In some embodiments, a first staple is formed after an amino acid residue comprising one of the third and fourth reactive group is installed but before an amino acid residue comprising the other of the third and fourth reactive group is installed. In some embodiments, two or more stapling steps are performed based on the positions of the related staples and the directions of peptide synthesis, and one or more staples closer to the starting termini are formed before one or more staples further away from the starting termini. In some embodiments, peptide synthesis is performed from C-terminus to N-terminus. In some embodiments, for a first staple and a second staple, the one that first has both related residues installed is formed first. For example, in a C-terminal to N-terminal peptide synthesis, a staple between X⁴ and X¹¹ is formed before a staple between X¹ and X⁴.

Various metal complexes or catalysts are useful for metathesis. For example, in some embodiments, a metal complex is a Grubbs catalyst. In some embodiments, it is In some embodiments, a metal complex is a Hoveyda-Grubbs catalyst. In some embodiments, it is Grubbs I M102. Hoveyda-Grubbs M720 catalyst. In some embodiments, a catalyst provides product E Z selectivity. As appreciated by those skilled in the art, catalysts can be utilized at various suitable levels, e.g., about 1%, 2%, 3%, 4%, 5%, 10%, 20%, 25%, 30%, 40%, 50% mol or more.

In some embodiments, the present disclosure provides technologies for controlling ratio of E/Z isomers of one or more or each olefin double bond formed during olefin metathesis. In some embodiments, one or more or each olefin double bond is formed with a isomer ratio of about 1.1:1, 1.2:1, 1.3:1, 1.4:1, 1.5:1, 2:1, 3:1, 4:1, 5:1, 10:1, 20:1, 30:1, 40:1, 50:1, or more. In some embodiments, in a product composition one or more or each olefin double bond has an isomer ratio of about 1.1:1, 1.2:1, 1.3:1, 1.4:1, 1.5:1, 2:1, 3:1, 4:1, 5:1, 10:1, 20:1, 30:1, 40:1, 50:1, or more. In some embodiments, it is independently about 1.5:1 or more. In some embodiments, it is independently about 2:1 or more. In some embodiments, it is independently about 3:1 or more. In some embodiments, it is independently about 4:1 or more. In some embodiments, it is independently about 5:1 or more. In some embodiments, it is independently about 6:1 or more. In some embodiments, it is independently about 7:1 or more. In some embodiments, it is independently about 8:1 or more. In some embodiments, it is independently about 9:1 or more. In some embodiments, it is independently about 10:1 or more. In some embodiments, it is independently about 20:1 or more. In some embodiments, it is independently about 30:1 or more. In some embodiments, it is independently about 40:1 or more. In some embodiments, it is independently about 50:1 or more. In some embodiments, a ratio is E:Z. In some embodiments, a ratio is Z:E.

In some embodiments, stapling creates one or more chiral centers. For example, in some embodiments, when B5 forms two staples with two other amino acid residues, a chiral center may form. In some embodiments, a formed chiral center is R in an agent. In some embodiments, a formed chiral center is S in an agent. In some embodiments, a composition comprises both agents being R and S at a chiral center. In some embodiments, a chiral center is formed with stereoselectivity (e.g., in some embodiments, diastereoselectivity when other chiral elements are present in the same molecule). In some embodiments, the selectivity is about or at least about 60%, 65%, 70%, 75%, 80%, 85%, 90%, 95%, 98% or 99% (when selectivity is 98%, 98% of all product molecules share the same stereochemistry at the chiral center.). In some embodiments, in a composition described herein, e.g., a pharmaceutical composition, about or at least about 60%, 65%, 70%, 75%, 80%, 85%, 90%, 95%, 98% or 99% of all molecules having the same constitution and salts thereof share the same stereochemistry at a chiral center, e.g., a chiral center bonded to two staples (e.g., in B5). In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 98%. In some embodiments, it is about or at least about 99%.

In some embodiments, an olefin double bond in a staple may be further modified. In some embodiments, an olefin double bond in a staple is hydrogenated thus converting it into a single bond. In some embodiments, a modification is epoxidation. In some embodiments, a modification is halogenation. Those skilled in the art appreciate that various other modifications are suitable for olefin double and can be utilized in accordance with the present disclosure.

In some embodiments, crude product compositions are purified, e.g., through chromatography technologies such as liquid chromatography. In some embodiments, one or more product compositions are collected based on separated portions, e.g., HPLC peaks, with the correct observed mass. In some embodiments, each product composition independently corresponds to a different peak (e.g., in some embodiments, by UV detection at a suitable wavelength, e.g., 220 nm) with the correct observed mass. In some embodiments, a peak area of one or more or each product composition is independently about 5%, 10%, 20%, 30%, 40%, 50%, 60%, 70%,80%, 90% or more of the total peak area of all peak(s) with the correct mass. In some embodiments, it is about 5% or more. In some embodiments, it is about 10% or more. In some embodiments, it is about 20% or more. In some embodiments, it is about 25% or more. In some embodiments, it is about 30% or more. In some embodiments, it is about 40% or more. In some embodiments, it is about 50% or more. In some embodiments, a product composition comprises one isomer. In some embodiments, a product composition comprises two or more isomers (e.g., those that cannot be sufficiently separated). In some embodiments, each product composition independently has a purity and/or stereopurity as described herein, e.g., in some embodiments, for one or more (e.g., 1, 2, 3, 4, 5 or more) or each olefin double bond in a staple, the ratio of the two stereoisomers is independently about 1.1:1, 1.2:1, 1.3:1, 1.4:1, 1.5:1, 2:1, 3:1, 4:1, 5:1, 10:1, 20:1, 30:1, 40:1, 50:1, 60:1, 70:1, 80:1, 90:1, 100:1 or more. In some embodiments, ratios may be assessed by NMR, HPLC, etc.

In some embodiments, as described herein, certain stapled peptides, and in particular cysteine stapled peptides, may be provided in and/or produced by a biological system and reacting with a provided reagent, e.g., one having the structure of R^{x}-L^{s2}-R^{x}, or a salt thereof, wherein R^{x} can react with —SH groups under suitable conditions. In some embodiments, each R^{x} is a suitable leaving group. In some embodiments, each R is independently —Br.

In some embodiments, peptides are prepared on solid phase on a synthesizer using, typically, Fmoc chemistry. In some embodiments, the present disclosure provides protected and/or activated amino acids for synthesis.

In some embodiments, staples are formed by olefin metathesis. In some embodiments, a product double bond of metathesis is reduced/hydrogenated. In some embodiments, CO₂ are extruded from a carbamate moiety of a staple. In some embodiments, provided stapled peptides are further modified, and/or conjugated to other entities. Conditions and/or reagents of these reactions are widely known in the art and can be performed in accordance with the present disclosure to provide stapled peptides.

Properties and/or activities of provided stapled peptides can be readily assessed in accordance with the present disclosure, for example, through use of one or more methods described in the examples.

In some embodiments, technologies for preparing and/or assessing provided stapled peptides include those described in U.S. Pat. No. 9,617,309, US 2015-0225471, US 2016-0024153, US 2016-0215036, US2016-0244494, WO 2017/062518, etc.

In some embodiments, the present disclosure provides products manufactured and/or characterized by processes and/or technologies described herein.

In some embodiments, a provided compound, e.g., an amino acid or a protected form thereof, may be prepared utilizing the following technologies.

In some embodiments, a provide compound may be prepared using one or more or all steps described below:

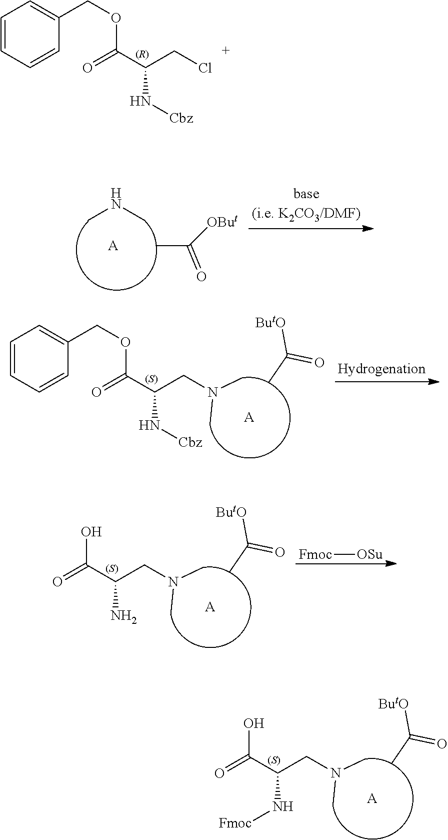

Those skilled in the art will appreciate that other leaving groups can be utilized in place of —Cl for the first reaction, such as —Br, —I, —OTs, Oms, etc.

In some embodiments, a provide compound may be prepared using one or more or all steps described below:

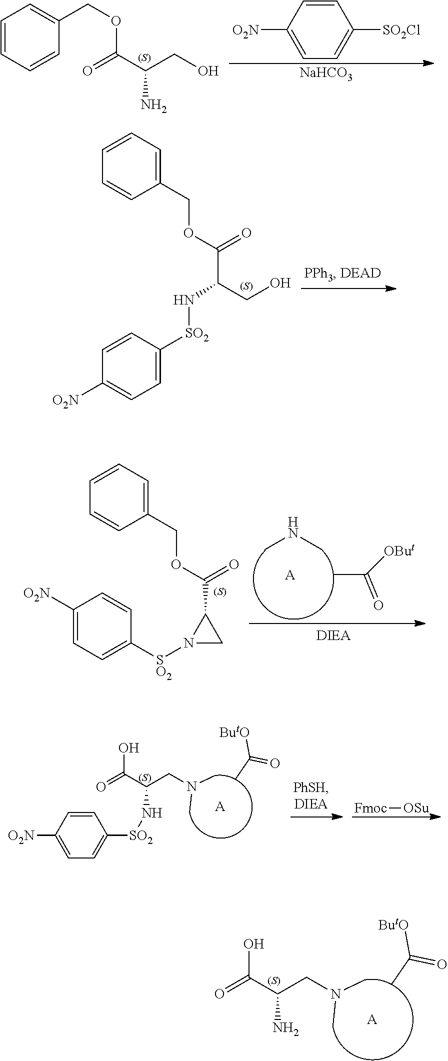

In some embodiments, a provide compound may be prepared using one or more or all steps described below:

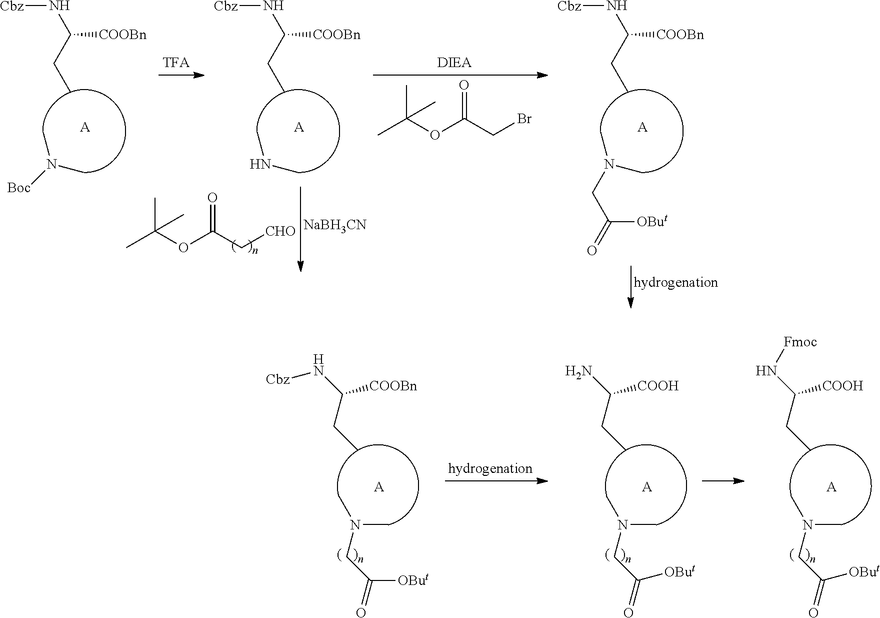

In some embodiments, a provide compound may be prepared using one or more or all steps described below:

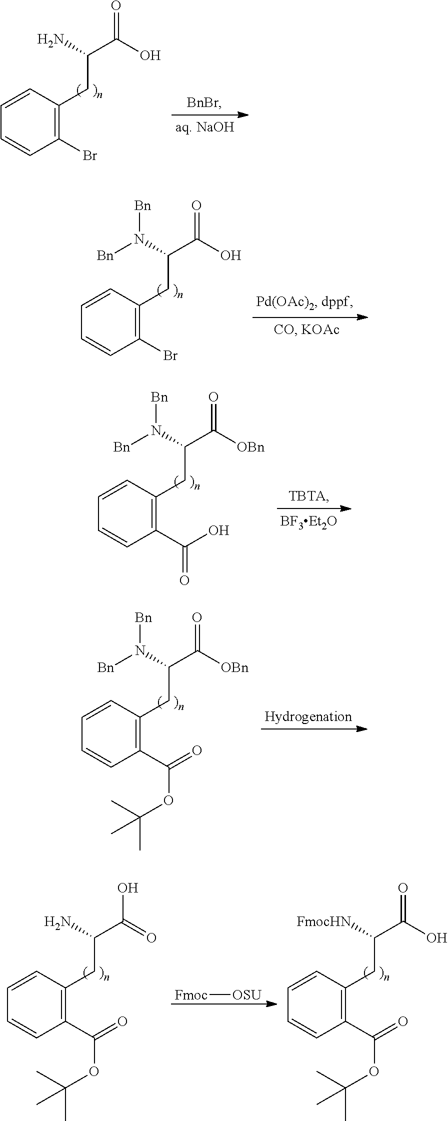

In some embodiments, a provide compound may be prepared using one or more or all steps described below:

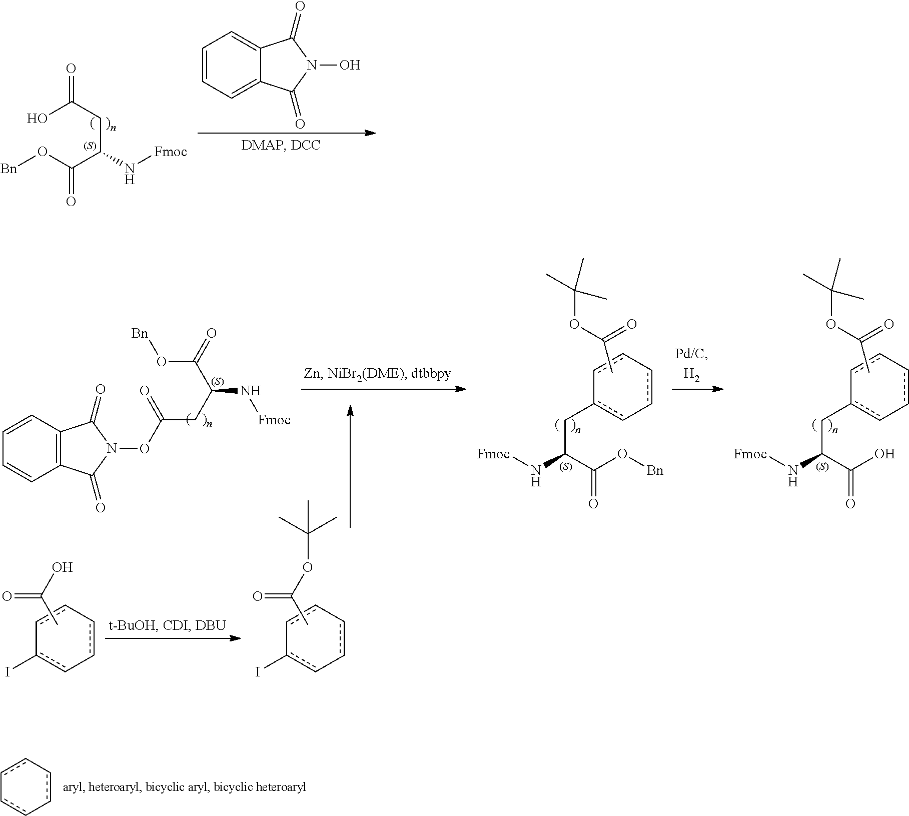

Provided compounds can be provided in high purity. In some embodiments, a provided compound is at least about 85%, 90%, 91%, 92%, 93%, 94%, 95%, 96%, 97%, 98%, or 99% pure. In some embodiments, provided compounds, e.g., amino acids optionally protected/activated, are essentially free of impurities, including stereoisomers.

In some embodiments, an agent may have one or more stereoisomers which may independently co-exist in a composition or preparation. In some embodiments, a provided agent has a stereopurity of about or at least about 80%, 85%, 90%, 91%, 92%, 93%, 94%, 95%, 96%, 97%, 98%, or 99%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%. In some embodiments, stereoisomers are essentially free from a preparation or composition (e.g., cannot be reliably observed in NMR or HPLC). In some embodiments, an agent comprises one or more staples independently comprising one or more olefin double bond. In some embodiments, stereopurity is with respect to E/Z stereoisomers. In some embodiments, for one or more (e.g., 1, 2, 3, 4, 5 or more) or each olefin double bond in a staple, the ratio of the two stereoisomers is independently about 1.1:1, 1.2:1, 1.3:1, 1.4:1, 1.5:1, 2:1, 3:1, 4:1, 5:1, 10:1, 20:1, 30:1, 40:1, 50:1, 60:1, 70:1, 80:1, 90:1, 100:1 or more. In some embodiments, it is independently about 1.5:1 or more. In some embodiments, it is independently about 2:1 or more. In some embodiments, it is independently about 3:1 or more. In some embodiments, it is independently about 4:1 or more. In some embodiments, it is independently about 5:1 or more. In some embodiments, it is independently about 6:1 or more. In some embodiments, it is independently about 7:1 or more. In some embodiments, it is independently about 8:1 or more. In some embodiments, it is independently about 9:1 or more. In some embodiments, it is independently about 10:1 or more. In some embodiments, it is independently about 20:1 or more. In some embodiments, it is independently about 30:1 or more. In some embodiments, it is independently about 40:1 or more. In some embodiments, it is independently about 50:1 or more. In some embodiments, it is independently about 60:1 or more. In some embodiments, it is independently about 70:1 or more. In some embodiments, it is independently about 80:1 or more. In some embodiments, it is independently about 90:1 or more. In some embodiments, it is independently about 100:1 or more. In some embodiments, a ratio is E:Z. In some embodiments, a ratio is Z:E. Those skilled in the art appreciate that E and Z isomers may be selectively enriched through modulating manufacturing processes, purification, staple positioning and/or lengths, etc.

### Compositions

Among other things, the present disclosure provides compositions that comprise or otherwise relate to provided agents, e.g., small molecule agents, peptide agents (e.g., stapled peptides), as described herein.

In some embodiments, provided compositions are or comprise an assay system for characterizing (and optionally including) a stapled peptide as described herein.

In some embodiments, provided compositions are pharmaceutical compositions e.g., that comprise or deliver one or more provided agents.

In some embodiments, an agent is a peptide. In some embodiments, an agent is a stapled peptide. In some embodiments, an agent comprises a detectable moiety, e.g., fluorescent moiety, radioactive moiety, biotin, etc. In some embodiments, a detectable moiety is directly detectable. In some embodiments, a detectable antibody is detected indirectly, e.g., utilizing an antibody, an agent that can reacting with a detectable moiety to form a detectable product, etc.

In some embodiments, a pharmaceutical composition comprises a provided agent and a pharmaceutically acceptable excipient (e.g., carrier).

In some embodiments, a peptide composition may include or deliver a particular form (e.g., a particular optical isomer, diastereomer, salt form, covalent conjugate form [e.g., covalently attached to a carrier moiety], etc., or combination thereof) of an agent as described herein). In some embodiments, an agent included or delivered by a pharmaceutical composition is described herein is not covalently linked to a carrier moiety.

In some embodiments, multiple stereoisomers exist for an agent that contains chiral centers and/or double bonds. In some embodiments, level of a particular agent in a composition is enriched relative to one or more or all of its stereoisomers. For example, in some embodiments, a particularly configuration of a double bond (E Z) is enrich. In some embodiments, for each double bond a configuration is independently enriched. In some embodiments, for a chiral element, e.g., a chiral center, one configuration is enriched. In some embodiments, for a chiral center bonded to two staples, one configuration is enriched. In some embodiments, for each chiral element a configuration is independently enriched. In some embodiments, for one or more or all stereochemical element (e.g., double bonds, chiral element, etc.), one configuration is independently enriched. In some embodiments, for each double bond in each staple, one configuration is independently enriched. In some embodiments, for each double bond in each staple, one configuration is independently enriched, and for a chiral center bonded to two staples, one configuration is enriched. In some embodiments, enrichment for each double bond is independently E or Z. In some embodiments, enrichment for each chiral element is independently R or S. In some embodiments, enrichment for each stereochemical element, e.g., double bond, chiral center, etc., is about or at least about a certain level, e.g., 60%, 65%, 70%, 75%, 80%, 85%, 90%, 91%, 92%, 93%, 94%, 95%, 96%, 97%, 98%, or 99% (percentage of an agent). In some embodiments, about or at least about a certain level, e.g., 60%, 65%, 70%, 75%, 80%, 85%, 90%, 91%, 92%, 93%, 94%, 95%, 96%, 97%, 98%, or 99% of all molecules in a composition that share the constitution of an agent or a salt thereof are the agent or a salt thereof. In some embodiments, a level is about or at least about 60%. In some embodiments, it is about or at least about 65%. In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%.

In some embodiments, a provided therapeutic composition may comprise one or more additional therapeutic agents and/or one or more stabilizing agents and/or one or more agents that alters (e.g., extends or limits to a particular tissue, location or site) rate or extent of delivery over time.

In some embodiments, a composition is a pharmaceutical composition which comprises or delivers a provided agent (e.g., a stapled peptide) or a pharmaceutically acceptable salt thereof and a pharmaceutically acceptable excipient. In some embodiments, a composition comprises one and only stereoisomer of an agent (e.g., a stapled peptide) and/or one or more salts thereof. In some embodiments, a composition comprises two or more stereoisomers of an agent (e.g., a stapled peptide) and/or one or more salts thereof. In some embodiments, the two or more stereoisomers of an agent (e.g., a stapled peptide) or salts thereof elute as a single peak (e.g., UV and/or MS detection) in a chromatography, e.g., HPLC.

### Uses and Applications

Provided agents and compositions can be utilized for various purposes. For example, certain compounds may be utilized as amino acids, either directly or for preparation of other compounds such as peptides. Certain agents, e.g., peptides, may be utilized to prepare stapled peptides. Certain agents that are or comprise peptides, particularly stapled peptides, and compositions thereof, are biologically active and can be utilized for various purposes, e.g., as therapeutics toward various conditions, disorders or diseases, as tools for modulating biological functions, etc.

In some embodiments, the present disclosure provides agents and compositions thereof for modulating beta-catenin functions. In some instances, beta-catenin is reported to have multiple cellular functions including regulation and coordination of cell-cell adhesion and gene transcription. In some embodiments, agents described herein may inhibit beta-catenin activity and/or level and may, for example, inhibit neoplastic growth. In some embodiments, agents described herein may activate and/or increase level of beta-catenin and may, for example, be used to treat male pattern baldness or alopecia.

It is reported that beta-catenin can interact with members of the TCF/LEF family at a TCF site on beta-catenin. In some embodiments, provided technologies can decrease, suppress or block one or more of such interactions. In some embodiments, the present disclosure provides methods for modulating an interaction between beta-catenin and its binding partner (e.g., a TCF/LEF family member) comprising contacting beta-catenin with a provided agent.

In some embodiments, binding of provided agents to beta-catenin competes or inhibits binding of another agent. In some embodiments, binding of provided agents to beta-catenin competes or inhibits binding of another agent. In some embodiments, binding of provided agents to beta-catenin competes or inhibits binding of TCF or a fragment thereof.

In some embodiments, provided agents compete with TCF7, LEF1, TCF7L1, TCF7L2, Axin1, Axin2, APC, CDH1, or CDH2, or a fragment thereof, for beta-catenin binding.

In some embodiments, provided agents interfere with interactions of TCF7, LEF1, TCF7L1, TCF7L2, Axin1, Axin2, APC, CDH1, or CDH2, or a fragment thereof, with beta-catenin.

In some embodiments, provided technologies can reduce or block beta-catenin's interactions with all TCF family members, E-cadherin and APC, but did not significantly affect its interactions with ICAT, AXIN and BCL9. In some embodiments, provided technologies can interrupt beta-catenin/TCF interaction at both physical interaction level (e.g., as confirmed by NanoBRET, co-IP, etc.) and transcriptional level (e.g., as confirmed by reporter cell line, endogenous gene expression, etc.). In some embodiments, provided technologies show no effect on beta-catenin stability.

In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising contacting beta-catenin with a provided agent or a composition that comprises or delivers a provided agent. In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising administering or delivering to a system comprising beta-catenin and the partner a provided agent or a composition that comprises or delivers a provided agent. In some embodiments, a system is an in vitro system. In some embodiments, a system is an in vivo system. In some embodiments, a system is or comprises a cell, tissue or organ. In some embodiments, a system is a subject. In some embodiments, the present disclosure provides method for inhibiting cell growth, comprising administering or delivering to a population of cells an effective amount of a provided agent or a pharmaceutically acceptable salt thereof. In some embodiments, the present disclosure provides method for killing cells associated with a condition, disorder or disease (e.g., cancer), comprising administering or delivering to a population of such cells an effective amount of a provided agent or a pharmaceutically acceptable salt thereof.

In some embodiments, the present disclosure provides methods for preventing a condition, disorder or disease associated with beta-catenin (e.g., a cancer, a neurodegenerative disease, etc.), comprising administering or delivering to a subject susceptible thereto an effective amount of a provided agent or a pharmaceutically acceptable salt thereof. In some embodiments, the present disclosure provides methods for treating a condition, disorder or disease associated with beta-catenin (e.g., aberrant beta-catenin activity and/or expression level), comprising administering or delivering to a subject suffering therefrom an effective amount of a provided agent or a pharmaceutically acceptable salt thereof. In some embodiments, a provided agent is administered as a pharmaceutical composition that comprises or delivers an effective amount of a provided agent or a pharmaceutically acceptable salt thereof. In some embodiments, a condition, disorder or disease is associated with beta-catenin interaction with a partner, e.g., TCF7, LEF1, TCF7L1, TCF7L2, Axin1, Axin2, APC, CDH1, and/or CDH2. In some embodiments, a condition, disorder or disease is associated with beta-catenin with TCF. In some embodiments, a condition, disorder or disease is cancer. In some embodiments, provided agents may be administered in combination with another therapy, e.g., immunotherapy. In some embodiments, a condition, disorder, or disease is selected from cancer, cardiac disease, dilated cardiomyopathy, fetal alcohol syndrome, depression, and diabetes. In some embodiments, a condition, disorder, or disease is a heart condition, disorder, or disease. In some embodiments, a condition, disorder, or disease is cancer. In some embodiments a cancer is selected from: colon cancer, colorectal cancer, rectal cancer, prostate cancer familial adenomatous polyposis (FAP), Wilms Tumor, melanoma, hepatocellular carcinoma, ovarian cancer, endometrial cancer, medulloblastoma pilomatricomas, primary hetpatocellular carcinoma, ovarial carcinoma, breast cancer, lung cancer, glioblastoma, pliomatrixoma, medulloblastoma, thyroid tumors, and ovarian neoplasms. In some embodiments, a condition, disorder or disease is a cancer, e.g., colorectal cancer, hepatocellular cancer, melanoma, gastric cancer, bladder cancer, and endometrial cancer. In some embodiments, a cancer is colorectal cancer. In some embodiments, a cancer is hepatocellular cancer. In some embodiments, a cancer is prostate cancer. In some embodiments, a cancer is melanoma.

In some embodiments, the present disclosure provides technologies for modulate level of expression and/or activity of a nucleic acid, e.g., a gene, a transcript, a polypeptide, and/or a product thereof in a system, comprising administering or delivering to the system a provided agent or a composition that comprises or delivers a provided agent. In some embodiments, level of expression of a nucleic acid, e.g., a gene, or a product thereof (e.g., a transcript, a polypeptide, etc.) is modulated. In some embodiments, level of activity of a nucleic acid, e.g., a gene, or a product thereof (e.g., a transcript, a polypeptide, etc.) is modulated. In some embodiments, level of a transcript and/or a product thereof (e.g., a polypeptide) is modulated. In some embodiments, level of activity of a transcript and/or a product thereof (e.g., a polypeptide) is modulated. In some embodiments, a transcript is a transcript of a nucleic acid, e.g., gene, described herein. In some embodiments, level of a polypeptide is modulated. In some embodiments, level of activity of a polypeptide is modulated. In some embodiments, a polypeptide is a encoded by a nucleic acid or a transcript described herein. In some embodiments, a level is increased. In some embodiments, a level is decreased. As described herein, in some embodiments, a system is an in vitro system. In some embodiments, a system is an in vivo system. In some embodiments, a system is or comprises a cell, tissue or organ. In some embodiments, a system is or comprises one or more cancer cells. In some embodiments, a system is or comprises tumor. In some embodiments, a system is or comprises an organism. In some embodiments, a system is a subject. In some embodiments, a system is a human. In some embodiments, a system comprises beta-catenin. In some embodiments, a system expresses beta-catenin. In some embodiments, a system comprises beta-catenin and a partner. In some embodiments, a system expresses beta-catenin and a partner. In some embodiments, a level is regulated by beta-catenin. In some embodiments, a level is regulated by WNT activation. In some embodiments, a level is regulated by beta-catenin/WNT signaling. In some embodiments, a level is regulated by interaction of beta-catenin and a partner. In some embodiments, interaction of beta-catenin and a partner is modulated, e.g., reduced, prevented, etc., by an agent, e.g., a stapled peptide, as described herein. For example, in some embodiments, a partner is TCF. In some embodiments, level of expression and/or activity of a nucleic acid and/or a product thereof is modulated. In some embodiments, a nucleic acid is AXIN2. In some embodiments, level of an AXIN2 transcript, e.g., mRNA, is reduced. In some embodiments, level of an AXIN2 polypeptide is reduced. In some embodiments, a nucleic acid is SP5. In some embodiments, level of an SP5 transcript, e.g., mRNA, is reduced. In some embodiments, level of an SP5 polypeptide is reduced. In some embodiments, a nucleic acid is CXCL12. In some embodiments, level of a CXCL12 transcript, e.g., mRNA, is increased. In some embodiments, level of a CXCL12 polypeptide is increased. In some embodiments, a nucleic acid is a member of a negatively enriched gene set observed in, or can be identified using technologies in, e.g., Example 17. In some embodiments, a nucleic acid is a member of BCAT_GDS748-UP gene set. In some embodiments, a nucleic acid is a member of BCAT.100-UP.V1-UP gene set. In some embodiments, a nucleic acid is a member of HALLMARK_WNT_BETA_CATENIN_SIGNALING gene set. In some embodiments, a nucleic acid is a member of RASHI_RESPONSE_TO_IONIZING_RADIATION_1 gene set. In some embodiments, a nucleic acid is a member of REACTOME_RRNA_PROCESSING gene set. In some embodiments, a nucleic acid is a member of HALLMARK_MYC_TARGETS_V1 gene set. In some embodiments, a nucleic acid is a member of HALLMARK_MYC_TARGETS_V2 gene set. In some embodiments, a nucleic acid is a member of HALLMARK_OXIDATIVE_PHOSPHORYLATION gene set. In some embodiments, a nucleic acid is a member of HALLMARK_E2F_TARGETS gene set. In some embodiments, a nucleic acid is a member of HALLMARK_TNFA_SIGNALING_VIA_NFKB gene set. Description of various gene sets can be found publicly, e.g., https://www.gsea-msigdb.org/gsea/msigdb/. In some embodiments, one or more or some or a majority of but not all nucleic acids or genes in a gene set is impacted in the same way, but overall a gene set can be negatively or positively enriched. In some embodiments, a nucleic acid is selected from Table GS1. In some embodiments, a nucleic acid is selected from Table GS2. In some embodiments, a nucleic acid is selected from Table GS3. In some embodiments, a nucleic acid is selected from Table GS4. In some embodiments, a nucleic acid is selected from Table GS5. In some embodiments, a nucleic acid is selected from Table GS6. In some embodiments, a nucleic acid is selected from Table GS7. In some embodiments, a nucleic acid is selected from Table GS8. In some embodiments, a nucleic acid is selected from Table GS9. In some embodiments, a nucleic acid is selected from Table GS10. In some embodiments, a nucleic acid is a gene selected Table GS1, Table GS2, Table GS3, Table GS4, Table GS5, Table GS6, Table GS7, Table GS8, Table GS9 or Table GS1O. In some embodiments, a gene is CCND2. In some embodiments, a gene is WNT5B. In some embodiments, a gene is AXIN2. In some embodiments, a gene is NKD1. In some embodiments, a gene is WNT6. In some embodiments, a gene is DKK1. In some embodiments, a gene is DKK4. In some embodiments, expression of such a nucleic acid, e.g., a gene, is reduced. In some embodiments, level of a product of such a nucleic acid, e.g., a transcript (e.g., mRNA), a polypeptide, etc., is reduced. In some embodiments, level of activity of a product of such a nucleic acid, e.g., a transcript (e.g., mRNA), a polypeptide, etc., is reduced.

TABLE GS1
Certain examples of nucleic acids including various members of BCAT_GDS748_UP.

| NCBI (Entrez) | Nucleic Acid/ | NCBI (Entrez) | Nucleic Acid/ | NCBI (Entrez) | Nucleic Acid / |
|---|---|---|---|---|---|
| Gene Id | Gene Symbol | Gene Id | Gene Symbol | Gene Id | Gene Symbol |
| 5243 | ABCB1 | 26281 | FGF20 | 4739 | NEDD9 |
| 202 | CRYBG1 | 2324 | FLT4 | 4884 | NPTX1 |
| 360 | AQP3 | 8324 | FZD7 | 5144 | PDE4D |
| 80150 | ASRGL1 | 2571 | GAD1 | 7262 | PHLDA2 |
| 9531 | BAG3 | 10912 | GADD45G | 5318 | PKP2 |
| 25805 | BAMBI | 2643 | GCH1 | 55041 | PLEKHB2 |
| 79669 | C3orf52 | 2650 | GCNT1 | 10394 | PRG3 |
| 84909 | AOPEP | 3087 | HHEX | 25797 | QPCT |
| 760 | CA2 | 3680 | ITGA9 | 861 | RUNX1 |
| 842 | CASP9 | 115207 | KCTD12 | 23516 | SLC39A14 |
| 1051 | CEBPB | 3823 | KLRC3 | 6520 | SLC3A2 |
| 23406 | COTL1 | 51176 | LEF1 | | SPRY4 |
| 23576 | DDAH1 | 4005 | LMO2 | 8406 | SRPX |
| 1670 | DEFA5 | | LSM12 | 9540 | TP5313 |
| 1780 | DYNC1I1 | 58530 | LY6G6D | 7334 | UBE2N |
| 2184 | FAH | 4488 | MSX2 | 7481 | WNT11 |
| 10447 | FAM3C | 4602 | MYB | | |

TABLE GS2
Certain examples of nucleic acids including various members of BCAT.100_UP.V1_UP.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 5243 | ABCB1 | 2202 | EFEMP1 | 3929 | LBP |
| 7984 | ARHGEF5 | 8507 | ENC1 | 4005 | LMO2 |
| 638 | BIK | 2026 | ENO2 | 85452 | CFAP74 |
| 652 | BMP4 | 5168 | ENPP2 | 58530 | LY6G6D |
| 55640 | FLVCR2 | 2119 | ETV5 | 79156 | PLEKHF1 |
| 928 | CD9 | 26281 | FGF20 | 5502 | PPPIRIA |
| 1045 | CDX2 | 10367 | MICU1 | 284119 | CAVIN1 |
| 140578 | CHODL | 2523 | FUT1 | 25797 | QPCT |
| 1428 | CRYM | 2571 | GAD1 | 5947 | RBP1 |
| 54440 | SASH3 | 10912 | GADD45G | 861 | RUNX1 |
| 79007 | DBNDD1 | 2650 | GCNT1 | 5274 | SERPINI1 |
| 1670 | DEFA5 | 3040 | HBA2 | 23428 | SLC7A8 |
| 22943 | DKK1 | 3198 | HOXA1 | 8470 | SORBS2 |
| 5611 | DNAJC3 | 8372 | HYAL3 | 6926 | TBX3 |
| 1846 | DUSP4 | 3549 | IHH | 7481 | WNT11 |
| 1848 | DUSP6 | 3667 | IRS1 | | |
| 1917 | EEF1A2 | 3680 | ITGA9 | | |

TABLE GS3
Certain examples of nucleic acids including various members
of HALLMARK_WNT_BETA_CATENIN_SIGNALING.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 6868 | ADAM17 | 2770 | GNAII | 85407 | NKD1 |
| 8312 | AXIN1 | 79885 | HDAC11 | 4851 | NOTCH1 |
| 8313 | AXIN2 | 3066 | HDAC2 | 4855 | NOTCH4 |
| 894 | CCND2 | 10014 | HDAC5 | 8650 | NUMB |
| 1454 | CSNKIE | 23462 | HEY1 | 5467 | PPARD |
| 1499 | CTNNB1 | 23493 | HEY2 | 5664 | PSEN2 |
| 8454 | CUL1 | 182 | JAG1 | 5727 | PTCH1 |
| 22943 | DKK1 | 3714 | JAG2 | 3516 | RBPJ |
| 27121 | DKK4 | 2648 | KAT2A | 6502 | SKP2 |
| 28514 | DLL1 | 51176 | LEF1 | 6932 | TCF7 |
| 1856 | DVL2 | 9794 | MAML1 | 7157 | TP53 |
| 10023 | FRAT1 | 4609 | MYC | 7471 | WNT1 |
| 8321 | FZD1 | 9612 | NCOR2 | 81029 | WNT5B |
| 8325 | FZD8 | 23385 | NCSTN | 7475 | WNT6 |

TABLE GS4
Certain examples of nucleic acids including various members of
RASHI_RESPONSE_TO_IONIZING_RADIATION_1.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 3725 | JUN | 7884 | SLBP | 1678 | TIMM8A |
| 1019 | CDK4 | 55192 | DNAJC17 | 55088 | CCDC186 |
| 8433 | UTF1 | 2353 | FOS | 1687 | GSDME |
| 1647 | GADD45A | 94234 | FOXQ1 | 50486 | GOS2 |
| 317772 | H2AC21 | 7538 | ZFP36 | 3290 | HSD11B1 |
| 286826 | LIN9 | 154 | ADRB2 | 6446 | SGK1 |
| 54361 | WNT4 | 7535 | ZAP70 | 7203 | CCT3 |
| 7422 | VEGFA | 6513 | SLC2A1 | 1958 | EGR1 |
| 92906 | HNRNPLL | 8870 | IER3 | 151295 | SLC23A3 |
| 11060 | WWP2 | 5362 | PLXNA2 | 5049 | PAFAHIB2 |
| 467 | ATF3 | 6271 | S100A1 | 9592 | IER2 |
| 1843 | DUSP1 | 54663 | WDR74 | 51503 | CWC15 |
| 10484 | SEC23A | 5269 | SERPINB6 | 1306 | COL15A1 |

TABLE GS5
Certain examples of nucleic acids including various
members of REACTOME_RRNA_PROCESSING.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 81887 | LASIL | 10436 | EMG1 | 51504 | TRMT112 |
| 60528 | ELAC2 | 6192 | RPS4Y1 | 54931 | TRMT10C |
| 115939 | TSR3 | 25873 | RPL36 | 6124 | RPL4 |
| 6224 | RPS20 | 23404 | EXOSC2 | 6138 | RPL15 |
| 51096 | UTP18 | 1736 | DKC1 | 6181 | RPLP2 |
| 51106 | TFB1M | 25926 | NOL11 | 6232 | RPS27 |
| 51121 | RPL26L1 | 6155 | RPL27 | 55272 | IMP3 |
| 23517 | MTREX | 57050 | UTP3 | 54913 | RPP25 |
| 51602 | NOP58 | 51388 | NIP7 | 54512 | EXOSC4 |
| 55781 | RIOK2 | 6210 | RPS15A | 55505 | NOP10 |
| 6141 | RPL18 | 134430 | WDR36 | 6218 | RPS17 |
| 10885 | WDR3 | 55226 | NAT10 | 6165 | RPL35A |
| 57418 | WDR18 | 84946 | LTV1 | 51118 | UTP11 |
| 55623 | THUMPD1 | 10200 | MPHOSPH6 | 10607 | TBL3 |
| 6160 | RPL31 | 92856 | IMP4 | 79050 | NOC4L |
| 114049 | BUD23 | 54881 | TEX10 | 51065 | RPS27L |
| 3028 | HSD17B10 | 11224 | RPL35 | 6228 | RPS23 |
| 23016 | EXOSC7 | 6194 | RPS6 | 9045 | RPL14 |
| 56915 | EXOSC5 | 6176 | RPLP1 | 27341 | RRP7A |
| 22894 | DIS3 | 6229 | RPS24 | 10438 | CID |
| 6193 | RPS5 | 55759 | WDR12 | 6231 | RPS26 |
| 27292 | DIMT1 | 6123 | RPL3L | 6168 | RPL37A |
| 8602 | NOP14 | 6187 | RPS2 | 6136 | RPL12 |
| 22803 | XRN2 | 84916 | UTP4 | 6191 | RPS4X |
| 6128 | RPL6 | 28987 | NOB1 | 6147 | RPL23A |
| 6175 | RPLP0 | 27043 | PELP1 | 4736 | RPL10A |
| 6222 | RPS18 | 1453 | CSNK1D | 6170 | RPL39 |
| 23481 | PES1 | 6205 | RPS11 | 9277 | WDR46 |
| 4809 | SNU13 | 23521 | RPL13A | 9277 | WDR46 |
| 6122 | RPL3 | 6135 | RPL11 | 4550 | MT-RNR2 |
| 9692 | PRORP | 6202 | RPS8 | 4549 | MT-RNR1 |
| 10528 | NOP56 | 81875 | ISG20L2 | 6235 | RPS29 |
| 8780 | RIOK3 | 6233 | RPS27A | 51202 | DDX47 |
| 90459 | ERI1 | 6161 | RPL32 | 1454 | CSNK1E |
| 6217 | RPS16 | 6189 | RPS3A | 7311 | UBA52 |
| 2091 | FBL | 6167 | RPL37 | 6222 | RPS18 |
| 6223 | RPS19 | 55651 | NHP2 | 118460 | EXOSC6 |
| 6142 | RPL18A | 6134 | RPL10 | 6222 | RPS18 |
| 54555 | DDX49 | 6129 | RPL7 | 9277 | WDR46 |
| 55131 | RBM28 | 6130 | RPL7A | 9277 | WDR46 |
| 51010 | EXOSC3 | 10556 | RPP30 | 6171 | RPL41 |
| 6158 | RPL28 | 22984 | PDCD11 | 6222 | RPS18 |
| 6143 | RPL19 | 6188 | RPS3 | 6234 | RPS28 |
| 117246 | FTSJ3 | 2197 | FAU | 6222 | RPS18 |
| 55813 | UTP6 | 57647 | DHX37 | 9277 | WDR46 |
| 6164 | RPL34 | 10557 | RPP38 | 79897 | RPP21 |
| 54433 | GAR1 | 6156 | RPL30 | 79897 | RPP21 |
| 6207 | RPS13 | 10813 | UTP14A | 6173 | RPL36A |
| 11103 | KRR1 | 8568 | RRP1 | 79897 | RPP21 |
| 4839 | NOP2 | 6132 | RPL8 | 79897 | RPP21 |
| 6206 | RPS12 | 26168 | SENP3 | 79897 | RPP21 |
| 705 | BYSL | 6154 | RPL26 | 5822 | PWP2 |
| 6152 | RPL24 | 6159 | RPL29 | 79897 | RPP21 |
| 9136 | RRP9 | 79707 | NOL9 | 79897 | RPP21 |
| 4691 | NCL | 200916 | RPL22L1 | 9724 | UTP14C |
| 6209 | RPS15 | 6133 | RPL9 | 23246 | BOP1 |
| 84128 | WDR75 | 11102 | RPP14 | 84916 | UTP4 |
| 56902 | PNO1 | 23160 | WDR43 | 6139 | RPL17 |
| 6146 | RPL22 | 116832 | RPL39L | 79159 | NOL12 |
| 10969 | EBNA1BP2 | 26354 | GNL3 | 6203 | RPS9 |
| 387338 | NSUN4 | 84135 | UTP15 | 6203 | RPS9 |
| 27042 | UTP25 | 6208 | RPS14 | 6203 | RPS9 |
| 6230 | RPS25 | 25879 | DCAF13 | 79922 | MRM1 |
| 55127 | HEATR1 | 65083 | NOL6 | 6203 | RPS9 |
| 51077 | FCF1 | 140801 | RPL10L | 6203 | RPS9 |
| 10171 | RCL1 | 6166 | RPL36AL | 6203 | RPS9 |
| 11340 | EXOSC8 | 9188 | DDX21 | 6203 | RPS9 |
| 27340 | UTP20 | 9790 | BMS1 | 11056 | DDX52 |
| 6144 | RPL21 | 6157 | RPL27A | 11056 | DDX52 |
| 130916 | MTERF4 | 6137 | RPL13 | 6203 | RPS9 |
| 6125 | RPL5 | 55720 | TSR1 | 6218 | RPS17 |
| 29960 | MRM2 | 3921 | RPSA | 6203 | RPS9 |
| 5393 | EXOSC9 | 6203 | RPS9 | 79922 | MRM1 |
| 10199 | MPHOSPH10 | 51013 | EXOSC1 | 2091 | FBL |
| 88745 | RRP36 | 5394 | EXOSC10 | 6230 | RPS25 |
| 6204 | RPS10 | 6227 | RPS21 | 6130 | RPL7A |
| 83732 | RIOK1 | 55178 | MRM3 | 140032 | RPS4Y2 |
| 10799 | RPP40 | 6201 | RPS7 | 23246 | BOP1 |
| 9349 | RPL23 | 6169 | RPL38 | 79050 | NOC4L |

TABLE GS6
Certain examples of nucleic acids including various
members of HALLMARK_MYC_TARGETS_V1.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 6059 | ABCE1 | 3251 | HPRT1 | 11137 | PWP1 |
| 52 | ACP1 | 3326 | HSP90AB1 | 5887 | RAD23B |
| 7965 | AIMP2 | 3329 | HSPD1 | 5901 | RAN |
| 1176 | AP3S1 | 3336 | HSPE1 | 5902 | RANBP1 |
| 328 | APEX1 | 3376 | LARS1 | 5984 | RFC4 |
| 9184 | BUB3 | 3475 | IFRD1 | 10921 | RNPS1 |
| 708 | C1QBP | 3608 | ILF2 | 9045 | RPL14 |
| 790 | CAD | 3615 | IMPDH2 | 6141 | RPL18 |
| 821 | CANX | 3735 | KARS1 | 6146 | RPL22 |
| 11335 | CBX3 | 3838 | KPNA2 | 6164 | RPL34 |
| 890 | CCNA2 | 3837 | KPNB1 | 6128 | RPL6 |
| 10576 | CCT2 | 3939 | LDHA | 6175 | RPLPO |
| 7203 | CCT3 | 57819 | LSM2 | 6204 | RPS10 |
| 10575 | CCT4 | 51690 | LSM7 | 6187 | RPS2 |
| 22948 | CCT5 | 4085 | MAD2L1 | 6188 | RPS3 |
| 10574 | CCT7 | 4171 | MCM2 | 6193 | RPS5 |
| 991 | CDC20 | 4173 | MCM4 | 6194 | RPS6 |
| 8318 | CDC45 | 4174 | MCM5 | 6240 | RRM1 |
| 1017 | CDK2 | 4175 | MCM6 | 9136 | RRP9 |
| 1019 | CDK4 | 4176 | MCM7 | 26156 | RSLID1 |
| 1207 | CLNSIA | 6150 | MRPL23 | 10856 | RUVBL2 |
| 7555 | CNBP | 65005 | MRPL9 | 26135 | SERBP1 |
| 10987 | COPS5 | 28973 | MRPS18B | 6418 | SET |
| 9377 | COX5A | 4609 | MYC | 10291 | SF3A1 |
| 1478 | CSTF2 | 4673 | NAP1L1 | 23450 | SF3B3 |
| 1503 | CTPS1 | 4686 | NCBP1 | 5250 | SLC25A3 |
| 8454 | CUL1 | 22916 | NCBP2 | 6599 | SMARCC1 |
| 1537 | CYC1 | 4706 | NDUFAB1 | 6626 | SNRPA |
| 8886 | DDX18 | 55651 | NHP2 | 6627 | SNRPA1 |
| 9188 | DDX21 | 4830 | NME1 | 6629 | SNRPB2 |
| 7913 | DEK | 9221 | NOLC1 | 6632 | SNRPD1 |
| 1665 | DHX15 | 51491 | NOP16 | 6633 | SNRPD2 |
| 1854 | DUT | 10528 | NOP56 | 6634 | SNRPD3 |
| 1933 | EEF1B2 | 4869 | NPM1 | 6637 | SNRPG |
| 1964 | EIF1AX | 4953 | ODC1 | 6723 | SRM |
| 1965 | EIF2S1 | 4999 | ORC2 | 6732 | SRPK1 |
| 8894 | EIF2S2 | 5036 | PA2G4 | 6426 | SRSF1 |
| 8662 | EIF3B | 26986 | PABPC1 | 6427 | SRSF2 |
| 8664 | EIF3D | 8761 | PABPC4 | 6428 | SRSF3 |
| 8669 | EIF3J | 5093 | PCBP1 | 6432 | SRSF7 |
| 1973 | EIF4A1 | 5111 | PCNA | 6741 | SSB |
| 1977 | EIF4E | 5230 | PGK1 | 6742 | SSBP1 |
| 1982 | EIF4G2 | 5245 | PHB | 56910 | STARD7 |
| 7458 | EIF4H | 11331 | PHB2 | 10492 | SYNCRIP |
| 2058 | EPRS1 | 5425 | POLD2 | 23435 | TARDBP |
| 2079 | ERH | 54107 | POLE3 | 6950 | TCP1 |
| 2107 | ETF1 | 5478 | PPIA | 7027 | TFDP1 |
| 23016 | EXOSC7 | 5496 | PPMIG | 9868 | TOMM70 |
| 23196 | FAM120A | 10935 | PRDX3 | 6434 | TRA2B |
| 2091 | FBL | 10549 | PRDX4 | 10155 | TRIM28 |
| 10146 | G3BP1 | 26121 | PRPF31 | 7284 | TUFM |
| 2739 | GLO1 | 5634 | PRPS2 | 10907 | TXNL4A |
| 10399 | RACK1 | 5682 | PSMA1 | 7298 | TYMS |
| 26354 | GNL3 | 5683 | PSMA2 | 7307 | U2AF1 |
| 2806 | GOT2 | 5685 | PSMA4 | 10054 | UBA2 |
| 2935 | GSPT1 | 5687 | PSMA6 | 7324 | UBE2E1 |
| 3015 | H2AZ1 | 5688 | PSMA7 | 7332 | UBE2L3 |
| 3066 | HDAC2 | 5690 | PSMB2 | 7398 | USP1 |
| 51020 | HDDC2 | 5691 | PSMB3 | 7411 | VBP1 |
| 3068 | HDGF | 5704 | PSMC4 | 7416 | VDAC1 |
| 3178 | HNRNPA1 | 5706 | PSMC6 | 7419 | VDAC3 |
| 3181 | HNRNPA2B1 | 5707 | PSMD1 | 7514 | XPO1 |
| 220988 | HNRNPA3 | 10213 | PSMD14 | 11260 | XPOT |
| 3183 | HNRNPC | 5709 | PSMD3 | 2547 | XRCC6 |
| 3184 | HNRNPD | 5713 | PSMD7 | 7531 | YWHAE |
| 10236 | HNRNPR | 5714 | PSMD8 | 10971 | YWHAQ |
| 3192 | HNRNPU | 10728 | PTGES3 | | |

TABLE GS7
Certain examples of nucleic acids including various
members of HALLMARK MYC_TARGETS_V2.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 7965 | AIMP2 | 10199 | MPHOSPH10 | 10196 | PRMT3 |
| 705 | BYSL | 51154 | MRTO4 | 80324 | PUS1 |
| 11335 | CBX3 | 10514 | MYBBP1A | 10244 | RABEPK |
| 1019 | CDK4 | 4609 | MYC | 10171 | RCL1 |
| 79077 | DCTPP1 | 29078 | NDUFAF4 | 23223 | RRP12 |
| 8886 | DDX18 | 51388 | NIP7 | 9136 | RRP9 |
| 1844 | DUSP2 | 79050 | NOC4L | 6573 | SLC19A1 |
| 56915 | EXOSC5 | 9221 | NOLC1 | 3177 | SLC29A2 |
| 2193 | FARSA | 51491 | NOP16 | 6652 | SORD |
| 26354 | GNL3 | 4839 | NOP2 | 6723 | SRM |
| 83743 | GRWD1 | 10528 | NOP56 | 6832 | SUPV3L1 |
| 3099 | HK2 | 4869 | NPM1 | 9238 | TBRG4 |
| 3329 | HSPD1 | 5036 | PA2G4 | 6949 | TCOF1 |
| 3336 | HSPE1 | 23481 | PES1 | 64216 | TFB2M |
| 92856 | IMP4 | 5245 | PHB | 27346 | TMEM97 |
| 79711 | IPO4 | 5347 | PLK1 | 7374 | UNG |
| 81887 | LASIL | 10733 | PLK4 | 27340 | UTP20 |
| 9064 | MAP3K6 | 56342 | PPAN | 23160 | WDR43 |
| 4173 | MCM4 | 23082 | PPRC1 | 54663 | WDR74 |
| 4174 | MCM5 | | | | |

TABLE GS8
Certain examples of nucleic acids including various members
of HALLMARK_OXIDATIVE_PHOSPHORYLATION.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 22 | ABCB7 | 1743 | DLST | 4717 | NDUFC1 |
| 30 | ACAA1 | 1891 | ECH1 | 4718 | NDUFC2 |
| 10449 | ACAA2 | 1892 | ECHS1 | 4719 | NDUFS1 |
| 34 | ACADM | 1632 | ECI1 | 4720 | NDUFS2 |
| 36 | ACADSB | 2108 | ETFA | 4722 | NDUFS3 |
| 37 | ACADVL | 2109 | ETFB | 4724 | NDUFS4 |
| 38 | ACAT1 | 2110 | ETFDH | 4726 | NDUFS6 |
| 50 | ACO2 | 2230 | FDX1 | 374291 | NDUFS7 |
| 10939 | AFG3L2 | 2271 | FH | 4728 | NDUFS8 |
| 9131 | AIFM1 | 2395 | FXN | 4723 | NDUFV1 |
| 211 | ALAS1 | 2746 | GLUD1 | 4729 | NDUFV2 |
| 4329 | ALDH6A1 | 2806 | GOT2 | 23530 | NNT |
| 481 | ATP1B1 | 2821 | GPI | 4835 | NQO2 |
| 498 | ATP5F1A | 2879 | GPX4 | 4942 | OAT |
| 506 | ATP5F1B | 80273 | GRPEL1 | 4967 | OGDH |
| 509 | ATP5F1C | 3030 | HADHA | 4976 | OPA1 |
| 513 | ATP5F1D | 3032 | HADHB | 5018 | OXA1L |
| 514 | ATP5F1E | 3052 | HCCS | 5160 | PDHA1 |
| 515 | ATP5PB | 3028 | HSD17B10 | 5162 | PDHB |
| 516 | ATP5MC1 | 3313 | HSPA9 | 8050 | PDHX |
| 517 | ATP5MC2 | 27429 | HTRA2 | 5166 | PDK4 |
| 518 | ATP5MC3 | 3417 | IDH1 | 54704 | PDP1 |
| 10476 | ATP5PD | 3418 | IDH2 | 11331 | PHB2 |
| 521 | ATP5ME | 3419 | IDH3A | 5264 | PHYH |
| 522 | ATP5PF | 3420 | IDH3B | 23203 | PMPCA |
| 9551 | ATP5MF | 3421 | IDH3G | 5435 | POLR2F |
| 10632 | ATP5MG | 10989 | IMMT | 5447 | POR |
| 539 | ATP5PO | 81689 | ISCA1 | 10935 | PRDX3 |
| 537 | ATP6AP1 | 23479 | ISCU | 54884 | RETSAT |
| 533 | ATP6V0B | 3939 | LDHA | 55288 | RHOT1 |
| 527 | ATP6V0C | 3945 | LDHB | 89941 | RHOT2 |
| 8992 | ATP6V0E1 | 10128 | LRPPRC | 6389 | SDHA |
| 528 | ATP6V1C1 | 4129 | MAOB | 6390 | SDHB |
| 51382 | ATP6V1D | 4190 | MDH1 | 6391 | SDHC |
| 529 | ATP6V1E1 | 4191 | MDH2 | 6392 | SDHD |
| 9296 | ATP6V1F | 9927 | MFN2 | 8402 | SLC25A11 |
| 9550 | ATP6V1G1 | 4259 | MGST3 | 8604 | SLC25A12 |
| 51606 | ATP6V1H | 65003 | MRPL11 | 788 | SLC25A20 |
| 581 | BAX | 29088 | MRPL15 | 5250 | SLC25A3 |
| 593 | BCKDHA | 64981 | MRPL34 | 291 | SLC25A4 |
| 56898 | BDH2 | 51318 | MRPL35 | 292 | SLC25A5 |
| 51660 | MPC1 | 64963 | MRPS11 | 293 | SLC25A6 |
| 840 | CASP7 | 6183 | MRPS12 | 8803 | SUCLA2 |
| 1352 | COX10 | 64960 | MRPS15 | 8802 | SUCLG1 |
| 1353 | COX11 | 56945 | MRPS22 | 6832 | SUPV3L1 |
| 1355 | COX15 | 10884 | MRPS30 | 6834 | SURF1 |
| 10063 | COX17 | 9617 | MTRF1 | 10312 | TCIRG1 |
| 1327 | COX4I1 | 4552 | MTRR | 26519 | TIMM10 |
| 9377 | COX5A | 10651 | MTX2 | 26517 | TIMM13 |
| 1329 | COX5B | 4694 | NDUFA1 | 10440 | TIMM17A |
| 1337 | COX6A1 | 4695 | NDUFA2 | 92609 | TIMM50 |
| 1340 | COX6B1 | 4696 | NDUFA3 | 26521 | TIMM8B |
| 1345 | COX6C | 4697 | NDUFA4 | 26520 | TIMM9 |
| 1347 | COX7A2 | 4698 | NDUFA5 | 56993 | TOMM22 |
| 9167 | COX7A2L | 4700 | NDUFA6 | 9868 | TOMM70 |
| 1349 | COX7B | 4701 | NDUFA7 | 29796 | UQCR10 |
| 1350 | COX7C | 4702 | NDUFA8 | 10975 | UQCR11 |
| 1351 | COX8A | 4704 | NDUFA9 | 7381 | UQCRB |
| 1374 | CPT1A | 4706 | NDUFAB1 | 7384 | UQCRC1 |
| 1431 | CS | 4707 | NDUFB1 | 7385 | UQCRC2 |
| 1528 | CYB5A | 4708 | NDUFB2 | 7386 | UQCRFS1 |
| 1727 | CYB5R3 | 4709 | NDUFB3 | 7388 | UQCRH |
| 1537 | CYC1 | 4710 | NDUFB4 | 27089 | UQCRQ |
| 54205 | CYCS | 4711 | NDUFB5 | 7416 | VDAC1 |
| 1666 | DECR1 | 4712 | NDUFB6 | 7417 | VDAC2 |
| 1737 | DLAT | 4713 | NDUFB7 | 7419 | VDAC3 |
| 1738 | DLD | 4714 | NDUFB8 | | |

TABLE GS9
Certain examples of nucleic acids including various
members of HALLMARK_E2F_TARGETS.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 204 | AK2 | 3161 | HMMR | 10549 | PRDX4 |
| 81611 | ANP32E | 51155 | JPT1 | 5558 | PRIM2 |
| 25842 | ASF1A | 3184 | HNRNPD | 5591 | PRKDC |
| 55723 | ASF1B | 3364 | HUS1 | 5631 | PRPS1 |
| 29028 | ATAD2 | 3609 | ILF3 | 11168 | PSIP1 |
| 6790 | AURKA | 54556 | ING3 | 29893 | PSMC3IP |
| 9212 | AURKB | 10527 | IPO7 | 9232 | PTTG1 |
| 580 | BARD1 | 146909 | KIF18B | 29127 | RACGAP1 |
| 332 | BIRC5 | 3835 | KIF22 | 5810 | RAD1 |
| 672 | BRCA1 | 11004 | KIF2C | 5885 | RAD21 |
| 675 | BRCA2 | 24137 | KIF4A | 10111 | RAD50 |
| 84312 | BRMS1L | 3838 | KPNA2 | 10635 | RAD51AP1 |
| 701 | BUB1B | 3930 | LBR | 5889 | RAD51C |
| 23468 | CBX5 | 3978 | LIG1 | 5901 | RAN |
| 9133 | CCNB2 | 4001 | LMNB1 | 5902 | RANBP1 |
| 898 | CCNE1 | 51747 | LUC7L3 | 5931 | RBBP7 |
| 9738 | CCP110 | 55646 | LYAR | 5981 | RFC1 |
| 991 | CDC20 | 4085 | MAD2L1 | 5982 | RFC2 |
| 993 | CDC25A | 4171 | MCM2 | 5983 | RFC3 |
| 994 | CDC25B | 4172 | MCM3 | 10535 | RNASEH2A |
| 83461 | CDCA3 | 4173 | MCM4 | 6117 | RPA1 |
| 55143 | CDCA8 | 4174 | MCM5 | 6118 | RPA2 |
| 983 | CDK1 | 4175 | MCM6 | 6119 | RPA3 |
| 1019 | CDK4 | 4176 | MCM7 | 9125 | CNOT9 |
| 1026 | CDKN1A | 9833 | MELK | 6241 | RRM2 |
| 1027 | CDKN1B | 4288 | MKI67 | 6470 | SHMT1 |
| 1029 | CDKN2A | 4292 | MLH1 | 7884 | SLBP |
| 1031 | CDKN2C | 253714 | MMS22L | 8243 | SMC1A |
| 1033 | CDKN3 | 4361 | MRE11 | 9126 | SMC3 |
| 1062 | CENPE | 4436 | MSH2 | 10051 | SMC4 |
| 79019 | CENPM | 10797 | MTHFD2 | 79677 | SMC6 |
| 1111 | CHEK1 | 83463 | MXD3 | 6628 | SNRPB |
| 11200 | CHEK2 | 4605 | MYBL2 | 10615 | SPAG5 |
| 11113 | CIT | 4609 | MYC | 147841 | SPC24 |
| 1163 | CKS1B | 84316 | NAA38 | 57405 | SPC25 |
| 1164 | CKS2 | 4673 | NAP1L1 | 6426 | SRSF1 |
| 1434 | CSE1L | 4678 | NASP | 6427 | SRSF2 |
| 10664 | CTCF | 4683 | NBN | 6749 | SSRP1 |
| 1503 | CTPS1 | 9918 | NCAPD2 | 10274 | STAG1 |
| 1633 | DCK | 4830 | NME1 | 3925 | STMN1 |
| 64858 | DCLRE1B | 9221 | NOLC1 | 6839 | SUV39H1 |
| 79077 | DCTPP1 | 10528 | NOP56 | 10492 | SYNCRIP |
| 10212 | DDX39A | 11051 | NUDT21 | 10460 | TACC3 |
| 7913 | DEK | 57122 | NUP107 | 9238 | TBRG4 |
| 55635 | DEPDC1 | 9972 | NUP153 | 6941 | TCF19 |
| 81624 | DIAPH3 | 23165 | NUP205 | 7037 | TFRC |
| 9787 | DLGAP5 | 4999 | ORC2 | 8914 | TIMELESS |
| 1786 | DNMT1 | 23594 | ORC6 | 54962 | TIPIN |
| 29980 | DONSON | 5036 | PA2G4 | 7083 | TK1 |
| 79075 | DSCC1 | 10606 | PAICS | 7112 | TMPO |
| 1854 | DUT | 9924 | PAN2 | 7153 | TOP2A |
| 79733 | E2F8 | 5111 | PCNA | 7157 | TP53 |
| 8726 | EED | 23047 | PDS5B | 6434 | TRA2B |
| 1965 | EIF2S1 | 84844 | PHF5A | 9319 | TRIP13 |
| 9700 | ESPL1 | 5347 | PLK1 | 203068 | TUBB |
| 11340 | EXOSC8 | 10733 | PLK4 | 7283 | TUBG1 |
| 2146 | EZH2 | 5395 | PMS2 | 27338 | UBE2S |
| 9837 | GINS1 | 5411 | PNN | 29089 | UBE2T |
| 64785 | GINS3 | 23649 | POLA2 | 55148 | UBR7 |
| 84296 | GINS4 | 5424 | POLD1 | 7374 | UNG |
| 2935 | GSPT1 | 5425 | POLD2 | 7398 | USP1 |
| 3014 | H2AX | 10714 | POLD3 | 197335 | WDR90 |
| 3015 | H2AZ1 | 5426 | POLE | 7465 | WEE1 |
| 3070 | HELLS | 56655 | POLE4 | 7514 | XPO1 |
| 3159 | HMGA1 | 10248 | POP7 | 2547 | XRCC6 |
| 3148 | HMGB2 | 8493 | PPM1D | 9183 | ZW10 |
| 3149 | HMGB3 | 5511 | PPP1R8 | | |

TABLE GS10
Certain examples of nucleic acids including various members
of HALLMARK_TNFA_SIGNALING_VIA_NFKB.

| NCBI (Entrez) | Nucleic Acid/ | NCBI (Entrez) | Nucleic Acid/ | NCBI (Entrez) | Nucleic Acid/ |
|---|---|---|---|---|---|
| Gene Id | Gene Symbol | Gene Id | Gene Symbol | Gene Id | Gene Symbol |
| 19 | ABCA1 | 1647 | GADD45A | 5187 | PER1 |
| 374 | AREG | 4616 | GADD45B | 5209 | PFKFB3 |
| 467 | ATF3 | 2643 | GCH1 | 22822 | PHLDA1 |
| 490 | ATP2B1 | 2669 | GEM | 7262 | PHLDA2 |
| 2683 | B4GALT1 | 9945 | GFPT2 | 5328 | PLAU |
| 9334 | B4GALT5 | 1880 | GPR183 | 5329 | PLAUR |
| 597 | BCL2A1 | 1839 | HBEGF | 5341 | PLEK |
| 602 | BCL3 | 3280 | HES1 | 10769 | PLK2 |
| 604 | BCL6 | 3383 | ICAM1 | 56937 | PMEPA1 |
| 8553 | BHLHE40 | 23308 | ICOSLG | 10957 | PNRC1 |
| 329 | BIRC2 | 3398 | ID2 | 8613 | PLPP3 |
| 330 | BIRC3 | 9592 | IER2 | 23645 | PPP1R15A |
| 650 | BMP2 | 8870 | IER3 | 5734 | PTGER4 |
| 694 | BTG1 | 51278 | IER5 | 5743 | PTGS2 |
| 7832 | BTG2 | 64135 | IFIH1 | 5791 | PTPRE |
| 10950 | BTG3 | 3433 | IFIT2 | 5806 | PTX3 |
| 6347 | CCL2 | 3460 | IFNGR2 | 1827 | RCAN1 |
| 6364 | CCL20 | 3593 | IL12B | 5966 | REL |
| 6351 | CCL4 | 3601 | IL15RA | 5970 | RELA |
| 6352 | CCL5 | 3606 | IL18 | 5971 | RELB |
| 595 | CCND1 | 3552 | IL1A | 388 | RHOB |
| 57018 | CCNL1 | 3553 | IL1B | 8767 | RIPK2 |
| 9034 | CCRL2 | 51561 | IL23A | 127544 | RNF19B |
| 960 | CD44 | 3569 | IL6 | 6303 | SAT1 |
| 969 | CD69 | 3572 | IL6ST | 6385 | SDC4 |
| 941 | CD80 | 3575 | IL7R | 5055 | SERPINB2 |
| 9308 | CD83 | 3624 | INHBA | 5271 | SERPINB8 |
| 1026 | CDKN1A | 3659 | IRF1 | 5054 | SERPINE1 |
| 1051 | CEBPB | 8660 | IRS2 | 6446 | SGK1 |
| 1052 | CEBPD | 182 | JAG1 | 150094 | SIK1 |
| 8837 | CFLAR | 3725 | JUN | 9120 | SLC16A6 |
| 23529 | CLCF1 | 3726 | JUNB | 6515 | SLC2A3 |
| 1435 | CSF1 | 23135 | KDM6B | 11182 | SLC2A6 |
| 1437 | CSF2 | 7071 | KLF10 | 4088 | SMAD3 |
| 2919 | CXCL1 | 10365 | KLF2 | 8303 | SNN |
| 3627 | CXCL10 | 9314 | KLF4 | 9021 | SOCS3 |
| 6373 | CXCL11 | 1316 | KLF6 | 6648 | SOD2 |
| 2920 | CXCL2 | 687 | KLF9 | 8877 | SPHK1 |
| 2921 | CXCL3 | 8942 | KYNU | 80176 | SPSB1 |
| 6372 | CXCL6 | 3914 | LAMB3 | 8878 | SQSTM1 |
| 57007 | ACKR3 | 3949 | LDLR | 6776 | STAT5A |
| 3491 | CCN1 | 3976 | LIF | 10010 | TANK |
| 23586 | DDX58 | 9516 | LITAF | 6890 | TAP1 |
| 23258 | DENND5A | 23764 | MAFF | 7050 | TGIF1 |
| 11080 | DNAJB4 | 5606 | MAP2K3 | 25976 | TIPARP |
| 55332 | DRAM1 | 1326 | MAP3K8 | 7097 | TLR2 |
| 1843 | DUSP1 | 4082 | MARCKS | 3371 | TNC |
| 1844 | DUSP2 | 4170 | MCL1 | 7124 | TNF |
| 1846 | DUSP4 | 9242 | MSC | 7127 | TNFAIP2 |
| 1847 | DUSP5 | 4084 | MXD1 | 7128 | TNFAIP3 |
| 1906 | EDN1 | 4609 | MYC | 7130 | TNFAIP6 |
| 1942 | EFNA1 | 10135 | NAMPT | 25816 | TNFAIP8 |
| 1958 | EGR1 | 10725 | NFAT5 | 3604 | TNFRSF9 |
| 1959 | EGR2 | 4780 | NFE2L2 | 8744 | TNFSF9 |
| 1960 | EGR3 | 4783 | NFIL3 | 10318 | TNIP1 |
| 10938 | EHD1 | 4790 | NFKB1 | 79155 | TNIP2 |
| 10209 | EIF1 | 4791 | NFKB2 | 7185 | TRAF1 |
| 2114 | ETS2 | 4792 | NFKBIA | 10221 | TRIB1 |
| 2150 | F2RL1 | 4794 | NFKBIE | 9322 | TRIP10 |
| 2152 | F3 | 4814 | NINJ1 | 8848 | TSC22D1 |
| 24147 | FJX1 | 3164 | NR4A1 | 7280 | TUBB2A |
| 2353 | FOS | 4929 | NR4A2 | 7422 | VEGFA |
| 2354 | FOSB | 8013 | NR4A3 | 79693 | YRDC |
| 8061 | FOSL1 | 4973 | OLR1 | 65986 | ZBTB10 |
| 2355 | FOSL2 | 24145 | PANX1 | 80149 | ZC3H12A |
| 2526 | FUT4 | 5142 | PDE4B | 7538 | ZFP36 |
| 50486 | G0S2 | 10611 | PDLIM5 | | |

In some embodiments, a nucleic acid is a member of a positively enriched gene set observed in, or can be identified using technologies in, e.g., Example 17.

In some embodiments, the present disclosure provides technologies for detecting, monitoring and/or confirming efficacy of an agent, e.g., a stapled peptide, or a method, e.g., a method of treating a condition, disorder or disease, a method for modulating level of a transcript and/or a product and/or activity thereof, comprising assessing level of expression and/or activity of a nucleic acid, e.g., a gene, a transcript, a polypeptide, and/or a product thereof. In some embodiments, the present disclosure provides technologies for detecting, monitoring and/or confirming efficacy of an agent, e.g., a stapled peptide, comprising administering the agent to a subject, and assessing level of expression and/or activity of a nucleic acid, e.g., a gene, a transcript, a polypeptide, and/or a product thereof, in the subject. In some embodiments, the present disclosure provides technologies for detecting, monitoring and/or confirming efficacy of a method for treating a condition, disorder or disease in a subject, comprising assessing level of expression and/or activity of a nucleic acid, e.g., a gene, a transcript, a polypeptide, and/or a product thereof, in the subject. In some embodiments, a method is a method for treating a condition, disorder or disease associated with TCF-beta-catenin interaction in a subject. In some embodiments, a condition, disorder or disease is cancer as described herein. In some embodiments, the present disclosure provides technologies for selecting subjects for administration or delivery of an agent, e.g., stapled peptide agents described herein (e.g., for preventing or treating a condition, disorder or disease). In some embodiments, the present disclosure provides technologies for selecting subjects for continued administration or delivery of an agent, e.g., stapled peptide agents described herein (e.g., for preventing or treating a condition, disorder or disease) after one or more administrations or deliveries. In some embodiments, level of a transcript is assessed. In some embodiments, level of a polypeptide is assessed. In some embodiments, assessment is performed utilizing a sample or samples collected from a system or a subject. In some embodiments, a sample is collected during administration or delivery. In some embodiments, a sample is collected after administration or delivery. As described herein, in some embodiments, level of expression and/or activity of a nucleic acid and/or a product thereof is modulated. In some embodiments, a nucleic acid is AXIN2. In some embodiments, level of an AXIN2 transcript, e.g., mRNA, is reduced. In some embodiments, level of an AXIN2 polypeptide is reduced. In some embodiments, a nucleic acid is SP5. In some embodiments, level of an SP5 transcript, e.g., mRNA, is reduced. In some embodiments, level of an SP5 polypeptide is reduced. In some embodiments, a nucleic acid is CXCL12. In some embodiments, level of a CXCL12 transcript, e.g., mRNA, is increased. In some embodiments, level of a CXCL12 polypeptide is increased. In some embodiments, a nucleic acid is a member of a negatively enriched gene set observed in, or can be identified using technologies in, e.g., Example 17. In some embodiments, a nucleic acid is a member of BCAT_GDS748-UP gene set. In some embodiments, a nucleic acid is a member of BCAT.100-UP.V1-UP gene set. In some embodiments, a nucleic acid is a member of HALLMARK_WNT_BETA_CATENIN_SIGNALING gene set. In some embodiments, a nucleic acid is a member of RASHI_RESPONSE_TO_IONIZING_RADIATION_1 gene set. In some embodiments, a nucleic acid is a member of REACTOME_RRNA_PROCESSING gene set. In some embodiments, a nucleic acid is a member of HALLMARK_MYC_TARGETS_V1 gene set. In some embodiments, a nucleic acid is a member of HALLMARK_MYC_TARGETS_V2 gene set. In some embodiments, a nucleic acid is a member of HALLMARK_OXIDATIVE_PHOSPHORYLATION gene set. In some embodiments, a nucleic acid is a member of HALLMARK_E2F_TARGETS gene set. In some embodiments, a nucleic acid is a member of HALLMARK_TNFA_SIGNALING_VIA_NFKB gene set. In some embodiments, a nucleic acid is selected from Table GS1. In some embodiments, a nucleic acid is selected from Table GS2. In some embodiments, a nucleic acid is selected from Table GS3. In some embodiments, a nucleic acid is selected from Table GS4. In some embodiments, a nucleic acid is selected from Table GS5. In some embodiments, a nucleic acid is selected from Table GS6. In some embodiments, a nucleic acid is selected from Table GS7. In some embodiments, a nucleic acid is selected from Table GS8. In some embodiments, a nucleic acid is selected from Table GS9. In some embodiments, a nucleic acid is selected from Table GS10. In some embodiments, a nucleic acid is a gene selected Table GS1, Table GS2, Table GS3, Table GS4, Table GS5, Table GS6, Table GS7, Table GS8, Table GS9 or Table GS10. In some embodiments, a gene is CCND2. In some embodiments, a gene is WNT5B. In some embodiments, a gene is AXIN2. In some embodiments, a gene is NKD1. In some embodiments, a gene is WNT6. In some embodiments, a gene is DKK1. In some embodiments, a gene is DKK4. In some embodiments, expression of such a nucleic acid, e.g., a gene, is reduced. In some embodiments, level of a product of such a nucleic acid, e.g., a transcript (e.g., mRNA), a polypeptide, etc., is reduced. In some embodiments, level of activity of a product of such a nucleic acid, e.g., a transcript (e.g., mRNA), a polypeptide, etc., is reduced. In some embodiments, a nucleic acid is a member of a positively enriched gene set observed in, or can be identified using technologies in, e.g., Example 17. In some embodiments, if one or more desired reductions of expression and/or levels of transcripts and/or products thereof, and/or one or more desired negatively and/or positively enriched gene sets, are observed, administration or delivery continues. In some embodiments, administration or delivery continues as prior one(s). In some embodiments, administration or delivery continue with an adjusted dose level and/or regimen. In some embodiments, if desired reductions of expression and/or levels of transcripts and/or products thereof, and/or one or more desired negatively and/or positively enriched gene sets, are not observed, administration or delivery may be adjusted, and in some embodiments, discontinued. In some embodiments, as described herein, desired reductions of expression and/or levels of transcripts and/or products thereof comprise reductions of expression and/or levels of transcripts and/or products thereof of one or more or a majority of or all of SP5, CCND2, WNT5B, AXIN2, NKD1, WNT6, DKK1 and DKK4, nucleic acids of BCAT_GDS748-UP, BCAT.100-UP.V1-UP, HALLMARK_WNT_BETA_CATENIN_SIGNALING, RASHI_RESPONSE_TO_IONIZING_RADIATION_1, REACTOME_RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, HALLMARK_TNFA_SIGNALING_VIA_NFKB, and Table GS1, Table GS2, Table GS3, Table GS4, Table GS5, Table GS6, Table GS7, Table GS8, Table GS9 and Table GS10. In some embodiments, as described herein, desired increase of expression and/or levels of transcripts and/or products thereof comprise increase of expression and/or levels of transcripts and/or products thereof of CXCL12. In some embodiments, desired gene set enrichments comprise negative enrichment of one or more or all of BCAT_GDS748-UP, BCAT.100-UP.V1-UP, HALLMARK_WNT_BETA_CATENIN_SIGNALING, RASHI_RESPONSE_TO_IONIZING_RADIATION_1, REACTOME_RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, and HALLMARK_TNFA_SIGNALING_VIA_NFKB. In some embodiments, desired gene set enrichments comprise negative enrichment of one or more or all of the set in Table GS1, the set in Table GS2, the set in Table GS3, the set in Table GS4, the set in Table GS5, the set in Table GS6, the set in Table GS7, the set in Table GS8, the set in Table GS9, and the set in Table GS10. Those skilled in the art, e.g., those skilled in relevant clinical fields, reading the present disclosure will appreciate how to make decisions in accordance with the present disclosure.

In some embodiments, comparison is made to a reference. For example, reduction, increase, enrichment (negative or positive), changes, etc., are typically made to a suitable reference. In some embodiments, reduction, increase, enrichment (negative or positive), changes, etc., are to a reference assessment, in some embodiments, of a reference sample. In some embodiments, a reference assessment is or comprises assessment conducted prior to an administration or delivery of an agent. In some embodiments, a reference sample is collected prior to an administration or delivery of an agent. In some embodiments, a reference assessment is or comprises assessment conducted during an administration or delivery of an agent. In some embodiments, a reference sample is collected during an administration or delivery of an agent. In some embodiments, a reference assessment is or comprises assessment conducted after an administration or delivery of an agent. In some embodiments, a reference sample is collected after an administration or delivery of an agent. In some embodiments, a reference assessment is or comprises assessment conducted after an earlier administration or delivery of an agent. In some embodiments, a reference sample is collected after earlier an administration or delivery of an agent.

In some embodiments, a sample is an aliquot of material obtained or derived from a source of interest as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., broncheoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample comprise cancer cells. In some embodiments, a sample is obtained from a tumor. In some embodiments, a sample is obtained from a tumor in a patient.

In some embodiments, levels of two or more transcripts and/or products thereof may be assessed. In some embodiments, assessment is performed after one or more doses of agents, e.g., stapled peptides are administered or delivered to a subject. In some embodiments, if profiles, e.g., reduction, increase, etc., of one or more transcripts and/or products thereof matches those described herein, administration or delivery to a subject may continue. In some embodiments, if profiles, e.g., reduction, increase, etc., of one or more transcripts and/or products thereof matches those described herein, administration or delivery to a subject may be stopped and/or continued according to different dose levels and/or regimens.

Various technologies can be utilized in accordance with the present disclosure to formulate, distribute, administer or deliver provided technologies such as agents, peptides, compounds, compositions, etc. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, provided technologies are administered intravenously.

Among other things, the present disclosure provides various structural moieties including designed amino acid residues that can be utilized to optimize various properties and activities, stability, delivery, pharmacodynamics, pharmacokinetics, etc. to provide various dosage forms, dosage regimen, therapeutic windows, etc. In some embodiments, provided agents and compositions thereof may be utilized with improved dosage regimen and/or unit doses. In some embodiments, administration of provided agents are adjusted based on conditions, disorders or diseases and/or subpopulations. In some embodiments, administration and/or dosage regimen of provided technologies are adjusted according to certain biomarkers and genomic alterations.

Provided agents may deliver biological effects, e.g., therapeutic effects, via various mechanisms. In some embodiments, efficacy may be driven by AUC. In some embodiments, efficacy may be driven by Cmax.

In some embodiments, a provided agent is utilized in combination with another therapy. In some embodiments, a provided agent is utilized in combination with another therapeutic agent. In some embodiments, another therapy or therapeutic agent is administered prior to an administration or delivery of a provided agent. In some embodiments, another therapy or therapeutic agent is administered at about the same time as an administration or delivery of a provided agent. In some embodiments, a provided agent and another agent is in the same pharmaceutical composition. In some embodiments, another therapy or therapeutic agent is administered subsequently to an administration or delivery of a provided agent. In some embodiments, a subject is exposed to both a provided agent and another therapeutic agent. In some embodiments, both a provided agent and another agent can be detected in a subject. In some embodiments, a provided agent is administered before another agent is cleared out by a subject or vice versa. In some embodiments, a provided agent is administered within the half-life, or 2, 3, 4, 5 or 6 times of the half-life, of another agent or vice versa. In some embodiments, a subject is exposed to a therapeutic effect of a provided agent and a therapeutic effect of another therapeutic agent. In some embodiments, an agent may provide an effect after an agent is cleared out or metabolized by a subject. In some embodiments, a procedure, e.g., surgery, radiation, etc., may provide an effect after the procedure is completed.

In some embodiments, another therapy is a cancer therapy. In some embodiments, another therapy is or comprises surgery. In some embodiments, another therapy is or comprises radiation therapy. In some embodiments, another therapy is or comprises immunotherapy. In some embodiments, another therapeutic agent is or comprises a drug. In some embodiments, another therapeutic agent is or comprises a cancer drug. In some embodiments, another therapeutic agent is or comprises a chemotherapeutic agent. In some embodiments, another therapeutic agent is or comprises a hormone therapy agent. In some embodiments, another therapeutic agent is or comprises a kinase inhibitor. In some embodiments, another therapeutic agent is or comprises a checkpoint inhibitor (e.g., antibodies against PD-1, PD-L1, CTLA-4, etc.). In some embodiments, a provide agent can be administered with lower unit dose and/or total dose compared to being used alone. In some embodiments, another agent can be administered with lower unit dose and/or total dose compared to being used alone. In some embodiments, one or more side effects associated with administration of a provided agent and/or another therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

In some embodiments, another agent is a checkpoint inhibitor, an EGFR inhibitor, a VEGF inhibitor, a VEGFR inhibitor, a kinase inhibitor, or an anti-cancer drug.

In some embodiments, an additional agent is a checkpoint inhibitor. In some embodiments, an additional agent is an immune oncology agent. In some embodiments, an additional agent is an antibody against a checkpoint molecules. In some embodiments, an additional agent is an antibody of PD1, PDL-1, CTLA4, A2AR, B7-H3, B7-H4, BTLA, IDO, KIR, LAG3, TIM-s, C10orf54, etc. In some embodiments, an antibody is an anti-PD1 antibody. In some embodiments, an antibody is an anti-PD-L1 antibody. In some embodiments, an antibody is an anti-CTLA4.

In some embodiments, another agent is an EGFR inhibitor, e.g., erlotinib, gefitinib, lapatinib, panitumumab, vandetanib, cetuximab, etc. In some embodiments, another agent is an VEGF and/or VEGFR inhibitor, e.g., pazopanib, bevacizumab, sorafenib, sunitinib, axitinib, ponatinib, regorafenib, vandetanib, cabozantinib, ramucirumab, lenvatinib, ziv-aflibercept, etc. In some embodiments, another agent is a kinase inhibitor. In some embodiments, another therapeutic agent is a chemotherapeutic agent. In some embodiments, another therapeutic agent is an anti-cancer drug, e.g., cyclophosphamide, methotrexate, 5-fluorouracil (5-FU), doxorubicin, mustine, vincristine, procarbazine, prednisolone, dacarbazine, bleomycin, etoposide, cisplatin, epirubicin, capecitabine, folinic acid, actinomycin, all-trans retinoic acid, azacitidine, azathioprine, bortezomib, carboplatin, chlorambucil, cytarabine, daunorubicin, docetaxel, doxifluridine, fluorouracil, gemcitabine, hydroxyurea, idarubicin, imatinib, irinotecan, mechlorethamine, mercaptopurine, mitoxantrone, paclitaxel, pemetrexed, teniposide, tioguanine, topotecan, valrubicin, vinblastine, vindesine, vinorelbine, oxaliplatin, etc.

Among other things, the present disclosure provides the following Embodiments:
1. An agent having the structure of formula I:

R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I
or a salt thereof, wherein:
   R^{N} is a peptide, an amino protecting group or R′-L^{RN}-;
   each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
      a first R′ group and a second R′ group which are taken together to form -L^{s}- which is bonded to the atom to which a first R′ group is attached and the atom to which a second R′ group is attached; and
      a third R′ group and a fourth R′ group which are taken together to form -L^{s}- which is bonded to the atom to which a third R′ group is attached and the atom to which a fourth R′ group is attached;
   each L^{s} is independently -L^{s1}-L^{s}-L^{s3}- wherein each L^{s1}, L^{s2} and L^{s3} is independently L;
   L^{AA1} is an amino acid residue that comprises a side chain comprising an acidic or polar group;
   L^{AA2} is an amino acid residue that comprises a side chain comprising an acidic or polar group;
   L^{AA3} is an amino acid residue;
   L^{AA4} is an amino acid residue that comprises a side chain comprising an optionally substituted aromatic group;
   L^{AA5} is an amino acid residue that comprises a side chain comprising an optionally substituted aromatic group;
   L^{AA6} is an amino acid residue that comprises a side chain comprising an optionally substituted aromatic group;
   R^{C} is a peptide, a carboxyl protecting group, -L^{RC}-R′, —O-L^{RC}-R′ or —N(R′)-L^{RC}-R′;
   each of L^{RN} and L^{RC} is independently L;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently -L-R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
2. An agent having the structure of formula I:

R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I
or a salt thereof, wherein:
   R^{N} is a peptide, an amino protecting group or R′-L^{RN}-;
   each of L^{P1}, L^{P2}, L³, L^{P4}, L^{P5}, L⁶, and L^{P7} is independently L, wherein L^{P1}, L^{P2}, L³, L^{P4}, L^{P5}, L⁶, and L^{P7} comprise:
      a first R′ group and a second R′ group which are taken together to form -L^{s}- which is bonded to the atom to which a first R′ group is attached and the atom to which a second R′ group is attached; and
      a third R′ group and a fourth R′ group which are taken together to form -L^{s}- which is bonded to the atom to which a third R′ group is attached and the atom to which a fourth R′ group is attached;
   each L^{s} is independently -L^{s1}-L^{s2}-L^{s3}- wherein each L^{s1}, L^{s2} and L^{s3} is independently L;
   L^{AA1} is L^{AR}, wherein a methylene unit is replaced with —C(R′)(R^{AS})—, wherein R^{AA}s is -L^{AS1}-R^{AA1}, wherein R^{AA1} is —C₂R or —SO₂R;
   L^{AA2} is L^{AR}, wherein a methylene unit is replaced with —C(R′)(R^{AS})—, wherein R^{AA}s is -L^{AS2}-R^{AA2} wherein R^{AA2} is —CO₂R, or —SO₂R;
   L^{AA3} is L^{AR}, wherein a methylene unit is replaced with —C(R′)(R^{AS})—, wherein R^{AS} is -L^{AS3}-R^{AA3} wherein R^{AA3} is R′;
   L^{AA4} is L^{AR}, wherein a methylene unit is replaced with —C(R′)(R^{AS})—, wherein R^{AS} is -L^{AS4}-R^{AA4} wherein R^{AA}A4 is an optionally substituted group selected from 6-14 membered aryl or 5-14 membered heteroaryl having 1-6 heteroatoms;
   L^{AA5} is L^{AR}, wherein a methylene unit is replaced with —C(R′)(R^{AS})—, wherein R^{AS} is -L^{AS5}-R^{AA5} wherein R^{AA}A5 is an optionally substituted group selected from 6-14 membered aryl or 5-14 membered heteroaryl having 1-6 heteroatoms;
   L^{AA6} is L^{AR}, wherein a methylene unit is replaced with —C(R′)(R^{AS})—, wherein R^{AS} is -L^{AS6}-R^{AA6} wherein R^{AA}A6 is an optionally substituted group selected from 6-14 membered aryl or 5-14 membered heteroaryl having 1-6 heteroatoms; R^{C} is a peptide, a carboxyl protecting group, -L^{RC}-R′, —O-L^{RC}-R′ or —N(R′)-L^{RC}-R′;
   each of L^{RN} and L^{RC} is independently L;
   each L^{AR} is independently an optionally substituted, bivalent C₁-C₆ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each of L^{AS1}, L^{AS2}, L^{AS3}, L^{AS4}, L^{AS5}, and L^{AS6} is independently L^{AS};
   each R^{AS} is independently -L^{AS}-R′;
   each L^{AS} is independently a covalent bond or an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently -L-R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
3. The agent of any one of the preceding Embodiments, wherein a second R′ group and a third R′ group are attached to the same atom.
4. The agent of any one of the preceding Embodiments, wherein each of the first, second and fourth R′ groups is independently attached to a different atom.
5. The agent of any one of the preceding Embodiments, wherein L^{P1}, L^{P2}, L^{P3}, L^{P4}, L^{P5}, L^{P6}, and L^{P7}further comprise a fifth R′ group and a sixth R′ groups which are taken together to form -L^{s}- which is bonded to the atom to which a fifth R′ group is attached and the atom to which a sixth R′ group is attached.
6. The agent of any one of the preceding Embodiments, wherein each of the first, second, fourth, fifth and sixth R′ groups is independently attached to a different atom.
7. The agent of any one of the preceding Embodiments, wherein L^{P1}, L^{P2}, L^{P3}, L^{P4}, L^{P5}, L^{P6}, and L^{P7} further comprise a seventh R′ group and an eighth R′ groups which are taken together to form -L^{s}- which is bonded to the atom to which a seventh R′ group is attached and the atom to which an eighth R′ group is attached.
8. The agent of any one of the preceding Embodiments, wherein each of the first, second, fourth, fifth, sixth, seventh and eighth R′ groups is independently attached to a different atom.
9. The agent of any one of the preceding Embodiments, wherein L^{s} formed by taking the first and the second R′ groups together is a staple as described herein.
10. The agent of any one of the preceding Embodiments, wherein L^{s} formed by taking the first and the second R′ groups together has a length of 5-20 (e.g., 5, 6, 7, 8, 9, 10, 11, 12, 13, 14, 15, 16, 17, 18, 19, or 20) atoms.
11. The agent of any one of the preceding Embodiments, wherein L^{s} formed by taking the third and the fourth R′ groups together is a staple as described herein.
12. The agent of any one of the preceding Embodiments, wherein L^{s} formed by taking the third and the fourth R′ groups together has a length of 5-20 (e.g., 5, 6, 7, 8, 9, 10, 11, 12, 13, 14, 15, 16, 17, 18, 19, or 20) atoms.
13. The agent of any one of the preceding Embodiments, wherein L^{s} formed by taking the third and the fourth R′ groups together has a length of 10-20 (e.g., 10, 11, 12, 13, 14, 15, 16, 17, 18, 19, or 20) atoms.
14. The agent of any one of the preceding Embodiments, wherein L^{s} formed by taking the fifth and the sixth R′ groups together has a length of 5-20 (e.g., 5, 6, 7, 8, 9, 10, 11, 12, 13, 14, 15, 16, 17, 18, 19, or 20) atoms.
15. The agent of any one of the preceding Embodiments, wherein L^{s} formed by taking the seventh and the eighth R′ groups together has a length of 5-20 (e.g., 5, 6, 7, 8, 9, 10, 11, 12, 13, 14, 15, 16, 17, 18, 19, or 20) atoms.
16. The agent of any one of the preceding Embodiments, wherein L^{P1} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
17. The agent of any one of the preceding Embodiments, wherein the length of L^{P1} is 2-10 (2, 3, 4, 5, 6, 7, 8, 9, or 10) atoms.
18. The agent of any one of the preceding Embodiments, wherein one or more methylene units of L^{P1} are independently replaced with —N(R′)— or —C(O)—.
19. The agent of any one of the preceding Embodiments, wherein one or more methylene units of L^{P1} are independently replaced with —N(R′)— or —C(O)N(R′)—.
20. The agent of any one of the preceding Embodiments, wherein one or more methylene units of L^{P1} are independently replaced with —N(R′)—, —C(R′)₂, or —C(O)N(R′)—.
21. The agent of any one of the preceding Embodiments, wherein one or more methylene units of L^{P1} are independently replaced with —N(R′)—, and one or more methylene units of L^{P1} are independently replaced with —C(O)N(R′)—.
22. The agent of any one of the preceding Embodiments, wherein a methylene unit of L^{P1} is replaced with —C(R′)₂—, wherein one of the R′ groups is a first R′ group of the four R′ groups, or a methylene unit of L^{P1} is replaced with —N(R′)—, wherein the R′ group is a first R′ group of the four R′ groups.
23. The agent of any one of the preceding Embodiments, wherein a methylene unit of L^{P1} is replaced with —C(R′)₂—, wherein one of the R′ groups is a first R′ group of the four R′ groups.
24. The agent of any one of the preceding Embodiments, wherein L^{P1} is or comprises —[X]p-X¹—, wherein each X and X¹ is independently an amino acid residue, wherein p is 0-10, and X¹ is bonded to L^{AA}. 25. The agent of any one of the preceding Embodiments, wherein L^{P1} is or comprises —X¹—.
26. The agent of any one of the preceding Embodiments, wherein X¹ comprises the first R′ group of the four R′ groups.
27. The agent of any one of the preceding Embodiments, wherein L^{AA1} an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
28. The agent of any one of the preceding Embodiments, wherein L^{AA1} is —N(R′)—C(R′)(R^{AS})C(O)—.
29. The agent of any one of the preceding Embodiments, wherein L^{AA1} is —NH—C(R′)(R^{AS})C(O)—.
30. The agent of any one of the preceding Embodiments, wherein L^{AS1} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
31. The agent of any one of the preceding Embodiments, wherein L^{AS1} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
32. The agent of any one of the preceding Embodiments, wherein L^{AS1} is an optionally substituted, bivalent C₁-C₁₀ aliphatic group, wherein one or more methylene units of the group are optionally and independently replaced with —O—, —S—, or —N(R′)—.
33. The agent of any one of the preceding Embodiments, wherein L^{AS}i is an optionally substituted, bivalent C₁-C₁₀ alkylene group.
34. The agent of any one of the preceding Embodiments, wherein L^{AS}i is optionally substituted —CH₂—.
35. The agent of any one of the preceding Embodiments, wherein L^{AS}i is —CH₂—.
36. The agent of any one of the preceding Embodiments, wherein R^{AA1} is —CO₂R.
37. The agent of any one of the preceding Embodiments, wherein R^{AA1} is —CO₂H.
38. The agent of any one of the preceding Embodiments, wherein L^{AA1} is an amino acid residue that comprises a side chain comprising an acidic group.
39. The agent of any one of the preceding Embodiments, wherein L^{AA1} is X².
40. The agent of any one of the preceding Embodiments, wherein L^{P2} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
41. The agent of any one of the preceding Embodiments, wherein the length of L^{P2} is 2-10 (2, 3, 4, 5, 6, 7, 8, 9, or 10) atoms.
42. The agent of any one of the preceding Embodiments, wherein the length of L^{P2} is 6 atoms.
43. The agent of any one of the preceding Embodiments, wherein one or more methylene units of L^{P2} are independently replaced with —N(R′)— or —C(O)—.
44. The agent of any one of the preceding Embodiments, wherein one or more methylene units of L^{P2} are independently replaced with —N(R′)— or —C(O)N(R′)—.
45. The agent of any one of the preceding Embodiments, wherein one or more methylene units of L^{P2} are independently replaced with —N(R′)—, —C(R′)₂, or —C(O)N(R′)—.
46. The agent of any one of the preceding Embodiments, wherein one or more methylene units of L^{P2} are independently replaced with —N(R′)—, and one or more methylene units of L^{P2} are independently replaced with —C(O)N(R′)—.
47. The agent of any one of the preceding Embodiments, wherein L^{P2} is or comprises —[X]pX⁴[X]p′-, wherein each X and X⁴ is independently an amino acid residue, and each of p and p′ is independently 0-10.
48. The agent of any one of the preceding Embodiments, wherein L^{P2} is or comprises —[X]pX³X⁴[X]p′-, wherein each X, X³ and X⁴ is independently an amino acid residue, and each of p and p′ is independently 0-10.
49. The agent of any one of the preceding Embodiments, wherein L^{P2} is or comprises —X³X⁴—, wherein each X³ and X⁴ is independently an amino acid residue, and X⁴ is bonded to L^{AA2}.
50. The agent of any one of the preceding Embodiments, wherein a methylene unit of L^{P2} is replaced with —C(R′)₂—, wherein one of the R′ groups is the second R′ group and the other is the third R′ group of the four R′ groups.
51. The agent of any one of the preceding Embodiments, wherein X⁴ comprises —C(R′)₂—, wherein one of the R′ groups is the second R′ group and the other is the third R′ group of the four R′ groups.
52. The agent of any one of the preceding Embodiments, wherein the L^{s} formed by taking the first and the second R′ groups together has the structure of a L^{s} group bonded to X¹ and X⁴ as described herein.
53. The agent of any one of the preceding Embodiments, wherein the L^{s} formed by taking the third and the fourth R′ groups together has the structure of a L^{s} group bonded to X⁴ and X¹¹ as described herein.
54. The agent of any one of Embodiments 1-49, wherein a methylene unit of L^{P2} is replaced with —C(R′)₂—, wherein one of the R′ groups is the second R′ group.
55. The agent of any one of Embodiments 1-49, wherein X³ comprises —C(R′)₂—, wherein one of the R′ groups is the second R′ group.
56. The agent of any one of Embodiments 1-49 and 54-55, wherein a methylene unit of L^{P2} is replaced with —C(R′)₂—, wherein one of the R′ groups is the third R′ group.
57. The agent of any one of Embodiments 1-49 and 54-55, wherein X⁴ comprises —C(R′)₂—, wherein one of the R′ groups is the third R′ group.
58. The agent of any one of Embodiments 1-49, wherein a methylene unit of L^{P2} is replaced with —C(R′)₂—, wherein one of the R′ groups is the fifth R′ group.
59. The agent of any one of Embodiments 1-49, wherein X³ comprises —C(R′)₂—, wherein one of the R′ groups is the fifth R′ group.
60. The agent of any one of Embodiments 1-49, wherein a methylene unit of L^{P2} is replaced with —C(R′)₂—, wherein one of the R′ groups is the seventh R′ group.
61. The agent of any one of Embodiments 1-49, wherein X³ comprises —C(R′)₂—, wherein one of the R′ groups is the seventh R′ group.
62. The agent of any one of Embodiments 1-49, wherein a methylene unit of L^{P2} is replaced with —C(R′)₂—, wherein one of the R′ groups is the first R′ group.
63. The agent of any one of Embodiments 1-49, wherein X⁴ comprises —C(R′)₂—, wherein one of the R′ groups is the first R′ group.
64. The agent of any one of the preceding Embodiments, wherein L 15 an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
65. The agent of any one of the preceding Embodiments, wherein L^{AA2} is —N(R′)—C(R′)(R^{AS})C(O)—.
66. The agent of any one of the preceding Embodiments, wherein L^{AA2} is NH—C(R′)(R^{AS})C(O)—.
67. The agent of any one of the preceding Embodiments, wherein L^{AS2} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
68. The agent of any one of the preceding Embodiments, wherein L^{AS2} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
69. The agent of any one of the preceding Embodiments, wherein L^{AS2} is an optionally substituted, bivalent C₁-C₁₀ aliphatic group, wherein one or more methylene units of the group are optionally and independently replaced with —O—, —S—, or —N(R′)—.
70. The agent of any one of the preceding Embodiments, wherein L^{AS2} is an optionally substituted, bivalent C₁-C₁₀ alkylene group.
71. The agent of any one of the preceding Embodiments, wherein L^{AS2} is optionally substituted —CH₂—.
72. The agent of any one of the preceding Embodiments, wherein L^{AS2} is —CH₂—.
73. The agent of any one of the preceding Embodiments, wherein R^{AA2} is —CO₂R.
74. The agent of any one of the preceding Embodiments, wherein R^{AA2} is —CO₂H.
75. The agent of any one of the preceding Embodiments, wherein L^{A2} is an amino acid residue that comprises a side chain comprising an acidic group.
76. The agent of any one of the preceding Embodiments, wherein L^{AA2} is X⁵.
77. The agent of any one of the preceding Embodiments, wherein the length of L^{P3} is 0-10 (0, 1, 2, 3, 4, 5, 6, 7, 8, 9, or 10) atoms.
78. The agent of any one of the preceding Embodiments, wherein L^{P3} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
79. The agent of any one of the preceding Embodiments, wherein the length of L^{P3} is 2-10 (2, 3, 4, 5, 6, 7, 8, 9, or 10) atoms.
80. The agent of any one of the preceding Embodiments, wherein the length of L^{P3} is 6 atoms.
81. The agent of any one of the preceding Embodiments, wherein one or more methylene units of L^{P3} are independently replaced with —N(R′)— or —C(O)—.
82. The agent of any one of the preceding Embodiments, wherein one or more methylene units of L^{P3} are independently replaced with —N(R′)— or —C(O)N(R′)—.
83. The agent of any one of the preceding Embodiments, wherein one or more methylene units of L^{P3} are independently replaced with —N(R′)—, —C(R′)₂, or —C(O)N(R′)—.
84. The agent of any one of the preceding Embodiments, wherein one or more methylene units of L^{P3} are independently replaced with —N(R′)—, and one or more methylene units of L^{P3} are independently replaced with —C(O)N(R′)—.
85. The agent of any one of the preceding Embodiments, wherein L^{P3} is or comprises —[X]pX⁶X⁷[X]p′-, wherein each X, X⁶ and X⁷ is independently an amino acid residue, and each of p and p′ is independently 0-10.
86. The agent of any one of the preceding Embodiments, wherein L^{P3} is or comprises —X⁶X⁷—, wherein each X⁶ and X⁷ is independently an amino acid residue, and X⁷ is bonded to L^{AA3}.
87. The agent of any one of the preceding Embodiments, wherein a methylene unit of L^{P3} is replaced with —C(R′)₂—, wherein one of the R′ groups is the fifth, sixth, seventh or eighth R′ group.
88. The agent of any one of the preceding Embodiments, wherein X⁷ comprises —C(R′)₂—, wherein one of the R′ groups is the fifth, sixth, seventh or eighth R′ group.
89. The agent of any one of Embodiments 87-88, wherein the R′ group is the fifth R′ group.
90. The agent of any one of Embodiments 87-88, wherein the R′ group is the sixth R′ group.
91. The agent of any one of Embodiments 87-88, wherein the R′ group is the seventh R′ group.
92. The agent of any one of Embodiments 87-88, wherein the R′ group is the eighth R′ group.
93. The agent of any one of the preceding Embodiments, wherein L^{P3} is a covalent bond.
94. The agent of any one of the preceding Embodiments, wherein L^{A3} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
95. The agent of any one of the preceding Embodiments, wherein L^{AA3} is —N(R′)—C(R′)(R^{AS})C(O)—.
96. The agent of any one of the preceding Embodiments, wherein L^{AA3} is NH—C(R′)(R^{AS})C(O)—.
97. The agent of any one of the preceding Embodiments, L^{AS3} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
98. The agent of any one of the preceding Embodiments, wherein R^{AS} is -L^{AS3}-R^{AA3}, wherein L^{AS3} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
99. The agent of any one of the preceding Embodiments, wherein L^{AS3} is an optionally substituted, bivalent C₁-C₁₀ aliphatic group, wherein one or more methylene units of the group are optionally and independently replaced with —O—, —S—, or —N(R′)—.
100. The agent of any one of the preceding Embodiments, wherein L^{AS3} is an optionally substituted, bivalent C₁-C₁₀ alkylene group.
101. The agent of any one of the preceding Embodiments, wherein L^{AS3} is optionally substituted —CH₂—.
102. The agent of any one of the preceding Embodiments, wherein L^{AS3} is —CH₂—.
103. The agent of any one of the preceding Embodiments, wherein R^{AA3} is —CO₂R.
104. The agent of any one of the preceding Embodiments, wherein R^{AA3} is —CO₂H.
105. The agent of any one of the preceding Embodiments, wherein L^{AA3} is an amino acid residue that comprises a side chain comprising an acidic group.
106. The agent of any one of the preceding Embodiments, wherein L^{AA3} is X⁶.
107. The agent of any one of Embodiments 1-102, wherein L^{AA3} is an amino acid residue that comprises a hydrophobic side chain.
108. The agent of any one of Embodiments 1-102, wherein R^{AA3} is a hydrophobic group.
109. The agent of any one of Embodiments 1-102, wherein R^{AA3} is an optionally substituted C₁₋₆ aliphatic group.
110. The agent of any one of Embodiments 1-102, wherein R^{AA3} is a C₁₋₆ aliphatic group.
111. The agent of any one of Embodiments 1-102, wherein R^{AA3} is a C₁₋₆ alkyl group.
112. The agent of any one of Embodiments 1-102, wherein L^{AA3} is X⁸.
113. The agent of any one of the preceding Embodiments, wherein L^{P4} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
114. The agent of any one of the preceding Embodiments, wherein the length of L^{P4} is 0-10 (0, 1, 2, 3, 4, 5, 6, 7, 8, 9, or 10) atoms.
115. The agent of any one of the preceding Embodiments, wherein the length of L^{P4} is 2-10 (2, 3, 4, 5, 6, 7, 8, 9, or 10) atoms.
116. The agent of any one of the preceding Embodiments, wherein the length of L^{P4} is 6 atoms.
117. The agent of any one of the preceding Embodiments, wherein one or more methylene units of L^{P4} are independently replaced with —N(R′)— or —C(O)—.
118. The agent of any one of the preceding Embodiments, wherein one or more methylene units of L^{P4} are independently replaced with —N(R′)— or —C(O)N(R′)—.
119. The agent of any one of the preceding Embodiments, wherein one or more methylene units of L^{P4} are independently replaced with —N(R′)—, —C(R′)₂, or —C(O)N(R′)—.
120. The agent of any one of the preceding Embodiments, wherein one or more methylene units of L^{P4} are independently replaced with —N(R′)—, and one or more methylene units of L^{P4} are independently replaced with —C(O)N(R′)—.
121. The agent of any one of the preceding Embodiments, wherein L^{P4} is or comprises —[X]pX⁷X⁸[X]p′-, wherein each X, X⁷ and X⁸ is independently an amino acid residue, and each of p and p′ is independently 0-10.
122. The agent of any one of the preceding Embodiments, wherein L^{P4} is or comprises —X⁷X⁸—, wherein each X⁷ and X⁸ is independently an amino acid residue, and X⁸ is bonded to L^{AA4}.
123. The agent of any one of the preceding Embodiments, wherein a methylene unit of L^{P4} is replaced with —C(R′)₂—, wherein one of the R′ groups is the fifth, sixth, seventh or eighth R′ group.
124. The agent of any one of the preceding Embodiments, wherein X⁷ comprises —C(R′)₂—, wherein one of the R′ groups is the fifth, sixth, seventh or eighth R′ group.
125. The agent of any one of Embodiments 123-124, wherein the R′ group is the fifth R′ group.
126. The agent of any one of Embodiments 123-124, wherein the R′ group is the sixth R′ group.
127. The agent of any one of Embodiments 123-124, wherein the R′ group is the seventh R′ group.
128. The agent of any one of Embodiments 123-124, wherein the R′ group is the eighth R′ group
129. The agent of any one of Embodiments 1-112, wherein L^{P4} is a covalent bond.
130. The agent of any one of the preceding Embodiments, wherein L^{AA4} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
131. The agent of any one of the preceding Embodiments, wherein L^{AA4} is —N(R′)—C(R′)(R^{AS})C(O)—.
132. The agent of any one of the preceding Embodiments, wherein L^{AA4} is —NH—C(R′)(R^{AS})C(O)—.
133. The agent of any one of the preceding Embodiments, wherein L^{AS4} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
134. The agent of any one of the preceding Embodiments, wherein L^{AS4} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
135. The agent of any one of the preceding Embodiments, L^{AS4} is an optionally substituted, bivalent C₁-C₁₀ aliphatic group, wherein one or more methylene units of the group are optionally and independently replaced with —O—, —S—, or —N(R′)—.
136. The agent of any one of the preceding Embodiments, wherein L^{AS4} is an optionally substituted, bivalent C₁-C₁₀ alkylene group.
137. The agent of any one of the preceding Embodiments, wherein L^{AS4} is optionally substituted —CH₂—.
138. The agent of any one of the preceding Embodiments, wherein L^{AS4} is —CH₂—.
139. The agent of any one of the preceding Embodiments, wherein R^{AA4} is optionally substituted 6-14 membered aryl.
140. The agent of any one of the preceding Embodiments, wherein R^{AA4} is optionally substituted phenyl.
141. The agent of any one of the preceding Embodiments, wherein R^{AA4} is phenyl.
142. The agent of any one of Embodiments 1-138, wherein R^{AA4} is optionally substituted 5-14 membered heteroaryl having 1-6 heteroatoms.
143. The agent of any one of Embodiments 1-138, wherein R^{AA4} is optionally substituted 5-membered monocyclic heteroaryl having 1-4 heteroatoms.
144. The agent of any one of Embodiments 1-138, wherein R^{AA4} is optionally substituted

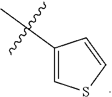 [SMILES: *c1ccsc1]

145. The agent of any one of Embodiments 1-138, wherein R^{AA4} is optionally substituted 9-membered bicyclic heteroaryl having 1-4 heteroatoms.
146. The agent of any one of Embodiments 1-138, wherein R^{AA4} is optionally substituted 10-membered bicyclic heteroaryl having 1-4 heteroatoms.
147. The agent of any one of Embodiments 1-138, wherein R^{AA4} is optionally substituted

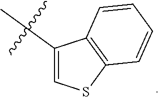 [SMILES: *c1csc2ccccc12]

148. The agent of any one of Embodiments 1-138, wherein R^{AA4} is optionally substituted

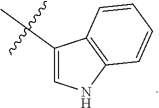 [SMILES: *c1c[nH]c2ccccc12]

149. The agent of any one of the preceding Embodiments, wherein L^{AA4} is an amino acid residue.
150. The agent of any one of the preceding Embodiments, wherein L^{AA4} is X⁹.
151. The agent of any one of the preceding Embodiments, wherein L^{P5} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
152. The agent of any one of the preceding Embodiments, wherein the length of L^{P5} is 2-10 (2, 3, 4, 5, 6, 7, 8, 9, or 10) atoms.
153. The agent of any one of the preceding Embodiments, wherein the length of L^{P5} is 6 atoms.
154. The agent of any one of the preceding Embodiments, wherein one or more methylene units of L^{P5} are independently replaced with —N(R′)— or —C(O)—.
155. The agent of any one of the preceding Embodiments, wherein one or more methylene units of L^{P5} are independently replaced with —N(R′)— or —C(O)N(R′)—.
156. The agent of any one of the preceding Embodiments, wherein one or more methylene units of L^{P5} are independently replaced with —N(R′)—, —C(R′)₂, or —C(O)N(R′)—.
157. The agent of any one of the preceding Embodiments, wherein one or more methylene units of L^{P5} are independently replaced with —N(R′)—, and one or more methylene units of L^{P5} are independently replaced with —C(O)N(R′)—.
158. The agent of any one of the preceding Embodiments, wherein a methylene unit of L^{P5} is replaced with —C(R′)₂—, wherein one of the R′ groups is the second or fourth R′ group.
159. The agent of any one of the preceding Embodiments, wherein L^{P5} is or comprises —[X]pX¹⁰X¹¹[X]p′-, wherein each X, X¹⁰ and X¹¹ is independently an amino acid residue, and each of p and p′ is independently 0-10.
160. The agent of any one of the preceding Embodiments, wherein L^{P5} is or comprises —X¹⁰X¹¹—, wherein each X¹⁰ and X¹¹ is independently an amino acid residue, and X¹¹ is bonded to L^{A}S.
161. The agent of any one of the preceding Embodiments, wherein X¹¹ comprises —C(R′)₂—, wherein one of the R′ groups is the second or fourth R′ group.
162. The agent of Embodiment 158 or 161, wherein one of the R′ groups is the second R′ group.
163. The agent of Embodiment 158 or 161, wherein one of the R′ groups is the fourth R′ group.
164. The agent of any one of the preceding Embodiments, wherein a methylene unit of L⁵ is replaced with —C(R′)₂—, wherein one of the R′ groups is the fifth, sixth, seventh or eighth R′ group.
165. The agent of any one of the preceding Embodiments, wherein X¹⁰ comprises —C(R′)₂—, wherein one of the R′ groups is the fifth, sixth, seventh or eighth R′ group.
166. The agent of any one of Embodiments 164-165, wherein the R′ group is the fifth R′ group.
167. The agent of any one of Embodiments 164-165, wherein the R′ group is the sixth R′ group.
168. The agent of any one of Embodiments 164-165, wherein the R′ group is the seventh R′ group.
169. The agent of any one of Embodiments 164-165, wherein the R′ group is the eighth R′ group.
170. The agent of any one of the preceding Embodiments, wherein L^{AA5} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
171. The agent of any one of the preceding Embodiments, wherein L^{AA5} is —N(R′)—C(R′)(R^{AS})C(O)—.
172. The agent of any one of the preceding Embodiments, wherein L^{AA5} is NH—C(R′)(R^{AS})C(O)—.
173. The agent of any one of the preceding Embodiments, wherein L^{AS5} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
174. The agent of any one of the preceding Embodiments, wherein L^{AS}s is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
175. The agent of any one of the preceding Embodiments, wherein L^{AS}s is an optionally substituted, bivalent C₁-C₁₀ aliphatic group, wherein one or more methylene units of the group are optionally and independently replaced with —O—, —S—, or —N(R′)—.
176. The agent of any one of the preceding Embodiments, wherein L^{AS}s is an optionally substituted, bivalent C₁-C₁₀ alkylene group.
177. The agent of any one of the preceding Embodiments, wherein L^{AS}s is optionally substituted —CH₂—.
178. The agent of any one of the preceding Embodiments, wherein L^{AS}s is —CH₂—.
179. The agent of any one of the preceding Embodiments, wherein R^{AA5} is optionally substituted 6-14 membered aryl.
180. The agent of any one of the preceding Embodiments, wherein R^{AA5} is optionally substituted phenyl.
181. The agent of any one of the preceding Embodiments, wherein R^{AA5} is phenyl.
182. The agent of any one of Embodiments 1-169, wherein R^{AA5} is optionally substituted 5-14 membered heteroaryl having 1-6 heteroatoms.
183. The agent of any one of Embodiments 1-169, wherein R^{AA5} is optionally substituted 5-membered monocyclic heteroaryl having 1-4 heteroatoms.
184. The agent of any one of Embodiments 1-169, wherein R^{AA5} is optionally substituted

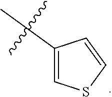 [SMILES: *c1ccsc1]

185. The agent of any one of Embodiments 1-169, wherein R^{AA5} is optionally substituted 9-membered bicyclic heteroaryl having 1-4 heteroatoms.
186. The agent of any one of Embodiments 1-169, wherein R^{AA5} is optionally substituted 10-membered bicyclic heteroaryl having 1-4 heteroatoms.
187. The agent of any one of Embodiments 1-169, wherein R^{AA5} is optionally substituted

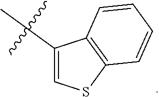 [SMILES: *c1csc2ccccc12]

188. The agent of any one of Embodiments 1-169, wherein R^{AA5} is optionally substituted

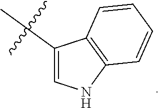 [SMILES: *c1c[nH]c2ccccc12]

189. The agent of any one of the preceding Embodiments, wherein L^{AA5} is an amino acid residue.
190. The agent of any one of the preceding Embodiments, wherein L^{AA5} is X¹².
191. The agent of any one of the preceding Embodiments, wherein L^{P6} is a covalent bond, or an optionally substituted, bivalent C₂-C₆ aliphatic group, wherein one or more methylene units of the group are optionally and independently replaced with —C(R′)₂—, -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
192. The agent of any one of the preceding Embodiments, wherein the length of L^{P6} is 0-10 (0, 1, 2, 3, 4, 5, 6, 7, 8, 9, or 10) atoms.
193. The agent of any one of the preceding Embodiments, wherein the length of L^{P6} is a covalent bond.
194. The agent of any one of the preceding Embodiments, wherein L^{AA6} is an optionally substituted, bivalent C₂-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
195. The agent of any one of the preceding Embodiments, wherein a methylene unit of L^{AA6} is replaced with —C(R′)(R^{AS})—, wherein R^{AS} is -L^{As}-R-A⁶, wherein L^{AS} is an optionally substituted, bivalent C₁-C₁₀ aliphatic group, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
196. The agent of any one of the preceding Embodiments, wherein a methylene unit of L^{AA6} is replaced with —C(R′)(R^{AS})—, wherein R^{AS} is -L^{As}-R-A⁶, wherein L^{AS} is an optionally substituted, bivalent C₁-C₁₀ aliphatic group, wherein one or more methylene units of the group are optionally and independently replaced with —C(R′)₂—, -Cy-, —O—, —S—, —N(R′)—, —C(O)—, —S(O)—, or —S(O)₂—.
197. The agent of any one of the preceding Embodiments, wherein a methylene unit of L^{AA6} is replaced with —C(R′)(R^{AS})—, wherein R^{AS} is -L^{As}-R-A⁶, wherein L^{AS} is an optionally substituted, bivalent C₁-C₁₀ aliphatic group, wherein one or more methylene units of the group are optionally and independently replaced with —O—, —S—, or —N(R′)—.
198. The agent of any one of the preceding Embodiments, wherein a methylene unit of L^{AA6} is replaced with —C(R′)(R^{AS})—, wherein R^{AS} is -L^{As}-R-A⁶, wherein L^{AS} is an optionally substituted, bivalent C₁-C₁₀ alkylene group.
199. The agent of any one of the preceding Embodiments, wherein a methylene unit of L^{AA6} is replaced with —C(R′)(R^{AS})—, wherein R^{AS} is —CH₂—R^{AA6}.
200. The agent of any one of the preceding Embodiments, wherein R^{AA6} is optionally substituted 6-14 membered aryl.
201. The agent of any one of the preceding Embodiments, wherein R^{AA6} is optionally substituted phenyl.
202. The agent of any one of the preceding Embodiments, wherein R^{AA6} is phenyl.
203. The agent of any one of Embodiments 1-193, wherein R^{AA6} is optionally substituted 5-14 membered heteroaryl having 1-6 heteroatoms.
204. The agent of any one of Embodiments 1-193, wherein R^{AA6} is optionally substituted 5-membered monocyclic heteroaryl having 1-4 heteroatoms.
205. The agent of any one of Embodiments 1-193, wherein R^{AA6} is optionally substituted

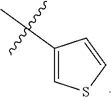 [SMILES: *c1ccsc1]

206. The agent of any one of Embodiments 1-193, wherein R^{AA6} is optionally substituted 9-membered bicyclic heteroaryl having 1-4 heteroatoms.
207. The agent of any one of Embodiments 1-193, wherein R^{AA6} is optionally substituted 10-membered bicyclic heteroaryl having 1-4 heteroatoms.
208. The agent of any one of Embodiments 1-193, wherein R^{AA6} is optionally substituted

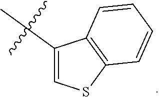 [SMILES: *c1csc2ccccc12]

209. The agent of any one of Embodiments 1-193, wherein R^{AA6} is optionally substituted

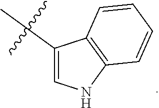 [SMILES: *c1c[nH]c2ccccc12]

210. The agent of any one of the preceding Embodiments, wherein L^{AA6} is an amino acid residue.
211. The agent of any one of the preceding Embodiments, wherein L^{AA6} is X¹³.
212. The agent of any one of the preceding Embodiments, wherein L^{P7} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
213. The agent of any one of the preceding Embodiments, wherein the length of L^{P7} is 0-20 (e.g., 0-15, 0-10, 0-5, 0, 1, 2, 3, 4, 5, 6, 7, 8, 9, 10, 11, 12, 13, 14, 15, 16, 17, 18, 19, or 20) atoms.
214. The agent of any one of the preceding Embodiments, wherein L^{P7} is or comprises —X¹⁴—[X]p′-, wherein p′ is 0-10, each of X and X¹⁴ is independently an amino acid residue, and X¹⁴ is bonded to L^{AA6}.
215. The agent of any one of the preceding Embodiments, wherein a methylene unit of L^{P7} is replaced with —C(R′)₂—, wherein one of the R′ groups is the sixth or eighth R′ group.
216. The agent of any one of the preceding Embodiments, wherein X¹⁴ comprises —C(R′)₂—, wherein one of the R′ groups is the fifth, sixth, seventh or eighth R′ group.
217. The agent of any one of Embodiments 215-216, wherein the R′ group is the sixth R′ group.
218. The agent of any one of Embodiments 215-216, wherein the R′ group is the eighth R′ group.
219. The agent of any one of the preceding Embodiments, wherein L^{RN} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
220. The agent of any one of the preceding Embodiments, wherein the length of L^{RN} is 0-20 (0, 1, 2, 3, 4, 5, 6, 7, 8, 9, 10, 11, 12, 13, 14, 15, 16, 17, 18, 19, or 20) atoms.
221. The agent of any one of the preceding Embodiments, wherein R^{N} is R′-L^{RN}-, wherein R′ is —C(O)R, —CO₂R, or —SO₂R.
222. The agent of any one of the preceding Embodiments, wherein R^{N} is R′, wherein R′ is —C(O)R, —CO₂R, or —SO₂R.
223. The agent of any one of the preceding Embodiments, wherein L^{RC} is a covalent bond, or an optionally substituted, bivalent C₁-C₁₀ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
224. The agent of any one of the preceding Embodiments, wherein the length of L^{RC} is 0-20 (0, 1, 2, 3, 4, 5, 6, 7, 8, 9, 10, 11, 12, 13, 14, 15, 16, 17, 18, 19, or 20) atoms.
225. The agent of any one of the preceding Embodiments, wherein R^{C} is —O-L^{RC}-R′ or —N(R′)-L^{RC}-R′.
226. The agent of any one of the preceding Embodiments, wherein R^{C} is —OR′ or —N(R′)₂, wherein each R′ is independently R.
227. An agent comprising one or more of:
   a first acidic group (e.g., of a first acidic amino acid residue);
   a second acidic group (e.g., of a second acidic amino acid residue);
   a first aromatic group (e.g., of a first aromatic amino acid residue);
   a second aromatic group (e.g., of a first aromatic amino acid residue); and
   a third aromatic group (e.g., of a third aromatic amino acid residue).
228. An agent comprising:
   a first acidic group (e.g., of a first acidic amino acid residue);
   a second acidic group (e.g., of a second acidic amino acid residue);
   a first aromatic group (e.g., of a first aromatic amino acid residue);
   a second aromatic group (e.g., of a first aromatic amino acid residue); and
   a third aromatic group (e.g., of a third aromatic amino acid residue).
229. An agent comprising:
   a first acidic group (e.g., of a first acidic amino acid residue);
   a second acidic group (e.g., of a second acidic amino acid residue);
   a third acidic group (e.g., of a third acidic amino acid residue);
   a first aromatic group (e.g., of a first aromatic amino acid residue);
   a second aromatic group (e.g., of a first aromatic amino acid residue); and
   a third aromatic group (e.g., of a third aromatic amino acid residue).
230. An agent comprising:
   a first acidic group (e.g., of a first acidic amino acid residue);
   a second acidic group (e.g., of a second acidic amino acid residue);
   a hydrophobic group (e.g., of a hydrophobic amino acid residue)
   a first aromatic group (e.g., of a first aromatic amino acid residue);
   a second aromatic group (e.g., of a first aromatic amino acid residue); and
   a third aromatic group (e.g., of a third aromatic amino acid residue).
231. An agent comprising:
   a first acidic group (e.g., of a first acidic amino acid residue);
   a second acidic group (e.g., of a second acidic amino acid residue);
   a third acidic group (e.g., of a third acidic amino acid residue);
   a hydrophobic group (e.g., of a hydrophobic amino acid residue)
   a first aromatic group (e.g., of a first aromatic amino acid residue);
   a second aromatic group (e.g., of a first aromatic amino acid residue); and
   a third aromatic group (e.g., of a third aromatic amino acid residue).
232. The agent of any one of Embodiments 227-231, wherein a first acidic group is of a first acidic amino acid residue.
233. The agent of any one of Embodiments 227-231, wherein a first acidic group is of L^{AA1} of any one of the preceding Embodiments.
234. The agent of any one of Embodiments 227-231, wherein a first acidic group is of a first acidic amino acid residue which is X².
235. The agent of any one of Embodiments 227-234, wherein a second acidic group is of a second acidic amino acid residue.
236. The agent of any one of Embodiments 227-234, wherein a second acidic group is of L^{AA2} of any one of the preceding Embodiments.
237. The agent of any one of Embodiments 227-234, wherein a second acidic group is of a second acidic amino acid residue which is X⁵.
238. The agent of any one of Embodiments 227-237, wherein a third acidic group is of a third acidic amino acid residue.
239. The agent of any one of Embodiments 227-237, wherein a third acidic group is of L^{AA3} of any one of the preceding Embodiments wherein L^{AA3} comprises an acidic group.
240. The agent of any one of Embodiments 227-237, wherein a third acidic group is of a third acidic amino acid residue which is X⁶.
241. The agent of any one of Embodiments 227-240, wherein a hydrophobic group is of a hydrophobic acidic amino acid residue.
242. The agent of any one of Embodiments 227-240, wherein a hydrophobic group is of L^{AA3} of any one of the preceding Embodiments wherein L^{AA3} comprises a hydrophobic group.
243. The agent of any one of Embodiments 227-240, wherein a hydrophobic group is of a hydrophobic acidic amino acid residue which is X.
244. The agent of any one of Embodiments 227-243, wherein a first aromatic group is of a first aromatic amino aromatic residue.
245. The agent of any one of Embodiments 227-243, wherein a first aromatic group is of L^{AA4} of any one of the preceding Embodiments.
246. The agent of any one of Embodiments 227-243, wherein a first aromatic group is of a first aromatic amino aromatic residue which is X⁹.
247. The agent of any one of Embodiments 227-246, wherein a second aromatic group is of a second aromatic amino aromatic residue.
248. The agent of any one of Embodiments 227-246, wherein a second aromatic group is of L^{AA5} ofany one of the preceding Embodiments.
249. The agent of any one of Embodiments 227-246, wherein a second aromatic group is of a second aromatic amino aromatic residue which is X².
250. The agent of any one of Embodiments 227-249, wherein a third aromatic group is of a third aromatic amino aromatic residue.
251. The agent of any one of Embodiments 227-249, wherein a third aromatic group is of L^{AA3} of any one of the preceding Embodiments wherein L^{AA6} comprises an aromatic group.
252. The agent of any one of Embodiments 227-249, wherein a third aromatic group is of a third aromatic amino aromatic residue which is X¹³.
253. The agent of any one of the preceding Embodiments, wherein the distance between a first acidic group and a second acidic group is about the distance between the acidic groups of two acidic amino acid residues of a peptide motif, wherein there are two amino acid residues between the two acidic amino acid residues.
254. The agent of any one of the preceding Embodiments, wherein a first acidic amino acid residue is at position N and a second is at position N+3.
255. The agent of any one of the preceding Embodiments, wherein the distance between a first acidic group and a third acidic group is about the distance between the acidic groups of two acidic amino acid residues of a peptide motif, wherein there are three amino acid residues between the two acidic amino acid residues.
256. The agent of any one of the preceding Embodiments, wherein a first acidic amino acid residue is at position N and a third is at position N+4.
257. The agent of any one of the preceding Embodiments, wherein the distance between a first acidic group and a hydrophobic group is about the distance between the acidic group of an acidic amino acid residue and the hydrophobic group of a hydrophobic amino acid residue of a peptide motif, wherein there are five amino acid residues between the first acidic amino acid residue and the hydrophobic amino acid residue.
258. The agent of any one of the preceding Embodiments, wherein a first acidic amino acid residue is at position N and a hydrophobic amino acid residue is at position N+6.
259. The agent of any one of the preceding Embodiments, wherein the distance between a first acidic group and a first aromatic group is about the distance between the acidic group of a first acidic amino acid residue and the aromatic group of an aromatic amino acid residue of a peptide motif, wherein there are six amino acid residues between the first acidic amino acid residue and the first aromatic amino acid residue.
260. The agent of any one of the preceding Embodiments, wherein a first acidic amino acid residue is at position N and a first aromatic amino acid residue is at position N+7.
261. The agent of any one of the preceding Embodiments, wherein the distance between the first aromatic group and the second aromatic group is about the distance between the aromatic groups of two aromatic amino acid residues of a peptide motif, wherein there are two amino acid residues between the two aromatic amino acid residues.
262. The agent of any one of the preceding Embodiments, wherein a first aromatic amino acid residue is at position M and a second is at position M+3.
263. The agent of any one of the preceding Embodiments, wherein the distance between the first aromatic group and the third aromatic group is about the distance between the aromatic groups of two aromatic amino acid residues of a peptide motif, wherein there are three amino acid residues between the two aromatic amino acid residues.
264. The agent of any one of the preceding Embodiments, wherein a first aromatic amino acid residue is at position N and a third is at position M+4).
265. The agent of any one of the preceding Embodiments, wherein N is 1-7.
266. The agent of any one of the preceding Embodiments, wherein N is 1, 2, 3, 4, or 5.
267. The agent of any one of the preceding Embodiments, wherein N is 1.
268. The agent of any one of the preceding Embodiments, wherein N is 2.
269. The agent of any one of the preceding Embodiments, wherein N is 3.
270. The agent of any one of the preceding Embodiments, wherein N is 4.
271. The agent of any one of the preceding Embodiments, wherein N is 5.
272. The agent of any one of the preceding Embodiments, wherein M is N+7.
273. The agent of any one of the preceding Embodiments, wherein M is 8-16.
274. The agent of any one of the preceding Embodiments, wherein M is 8.
275. The agent of any one of the preceding Embodiments, wherein M is 9.
276. The agent of any one of the preceding Embodiments, wherein M is 10.
277. The agent of any one of the preceding Embodiments, wherein M is 11.
278. The agent of any one of the preceding Embodiments, wherein M is 12.
279. The agent of any one of the preceding Embodiments, wherein M is 13.
280. The agent of any one of Embodiments 253-279, wherein the peptide motif is an alpha-helical motif wherein each amino acid residue is independently an alpha amino acid residue.
281. The agent of Embodiment 280, wherein the peptide motif is stapled.
282. The agent of Embodiment 281, wherein there are two staples in the peptide motif.
283. The agent of Embodiment 281, wherein there are three staples in the peptide motif.
284. The agent of Embodiment 281, wherein there are four staples in the peptide motif.
285. The agent of any one of Embodiments 253-279, wherein the peptide motif is or comprises an agent described in a Table herein (e.g., I-xxxx wherein xxxx is a number (e.g., I-1, I-10, I-100, I-1000, etc.)).
286. The agent of any one of the preceding Embodiments, wherein when the agent is contacted with a beta-catenin polypeptide, a first acidic group interacts with Lys312 or an amino acid residue corresponding thereto.
287. The agent of any one of the preceding Embodiments, wherein when the agent is contacted with a beta-catenin polypeptide, a first acidic group interacts with Gly307 or an amino acid residue corresponding thereto.
288. The agent of any one of the preceding Embodiments, wherein when the agent is contacted with a beta-catenin polypeptide, a second acidic group interacts with Asn387 or an amino acid residue corresponding thereto.
289. The agent of any one of the preceding Embodiments, wherein when the agent is contacted with a beta-catenin polypeptide, a second acidic group interacts with Trp383 or an amino acid residue corresponding thereto.
290. The agent of any one of the preceding Embodiments, wherein when the agent is contacted with a beta-catenin polypeptide, a third acidic group interacts with Tyr306 or an amino acid residue corresponding thereto.
291. The agent of any one of the preceding Embodiments, wherein when the agent is contacted with a beta-catenin polypeptide, a hydrophobic group interacts with Trp383 or an amino acid residue corresponding thereto.
292. The agent of any one of the preceding Embodiments, wherein when the agent is contacted with a beta-catenin polypeptide, a first aromatic group interacts with Lys345 or an amino acid residue corresponding thereto.
293. The agent of any one of the preceding Embodiments, wherein when the agent is contacted with a beta-catenin polypeptide, a first aromatic group interacts with Trp383 or an amino acid residue corresponding thereto.
294. The agent of any one of the preceding Embodiments, wherein when the agent is contacted with a beta-catenin polypeptide, a second aromatic group interacts with Trp383 or an amino acid residue corresponding thereto.
295. The agent of any one of the preceding Embodiments, wherein when the agent is contacted with a beta-catenin polypeptide, a second aromatic group interacts with Asn415 or an amino acid residue corresponding thereto.
296. The agent of any one of the preceding Embodiments, wherein when the agent is contacted with a beta-catenin polypeptide, a third aromatic group interacts with Gln379 or an amino acid residue corresponding thereto.
297. The agent of any one of the preceding Embodiments, wherein when the agent is contacted with a beta-catenin polypeptide, a third aromatic group interacts with Leu382 or an amino acid residue corresponding thereto.
298. The agent of any one of the preceding Embodiments, wherein when the agent is contacted with a beta-catenin polypeptide, a third aromatic group interacts with Val416 or an amino acid residue corresponding thereto.
299. The agent of any one of the preceding Embodiments, wherein when the agent is contacted with a beta-catenin polypeptide, a third aromatic group interacts with Asn415 or an amino acid residue corresponding thereto.
300. The agent of any one of the preceding Embodiments, wherein when the agent is contacted with a beta-catenin polypeptide, a third aromatic group interacts with Trp383 or an amino acid residue corresponding thereto.
301. The agent of any one of the preceding Embodiments, wherein the agent is or comprise a peptide.
302. The agent of any one of the preceding Embodiments, wherein the agent is a peptide.
303. The agent of any one of the preceding Embodiments, wherein the peptide is a stapled peptides comprising two or more staples.
304. The agent of any one of the preceding Embodiments, wherein the peptide is a stapled peptides comprising three or more staples.
305. The agent of any one of the preceding Embodiments, wherein the peptide is a stapled peptides comprising three and no more than three staples.
306. The agent of any one of the preceding Embodiments, wherein the peptide is a stapled peptides comprising four and no more than four staples.
307. The agent of any one of the preceding Embodiments, wherein a first acidic group, a second acidic group, a third acidic group, a hydrophobic group, a first aromatic group, a second aromatic group and a third aromatic group, if present, are presented from N to C direction of a peptide.
308. The agent of any one of the preceding Embodiments, wherein the agent is or comprises a helix structure.
309. An agent, comprising:

X¹X²X³X⁴X⁵X⁶X⁷X⁸X⁹X¹⁰X¹¹X¹²X¹³X¹⁴,
wherein:
   each of X¹, X², X³, X⁴, X⁵, X⁶, X⁷, X⁸, X⁹, X¹⁰, X¹¹, X¹², X³, and X¹⁴ is independently an amino acid residue, wherein:
   X² comprises a side chain comprising an acidic or a polar group;
   X⁵ comprises a side chain comprising an acidic or a polar group; and
   each of X⁹, X¹² and X¹³ comprises a side chain comprising an optionally substituted aromatic group.
310. An agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,
wherein:
   each of p0, p15, p16 and p17 is independently 0 or 1;
   each of X⁰, X¹, X², X³, X⁴, X⁵, X⁶, X⁷, X⁸, X⁹, X¹⁰, X¹¹, X¹², X¹³, X¹⁴, X⁵, X⁶, and X⁷ is independently an amino acid residue, wherein:
   X² comprises a side chain comprising an acidic or a polar group;
   X⁵ comprises a side chain comprising an acidic or a polar group; and
   each of X⁹, X¹² and X¹³ comprises a side chain comprising an optionally substituted aromatic group.
311. An agent, comprising:

X¹X²X³X⁴X⁵X⁶X⁷X⁸X⁹X¹⁰X¹¹X¹²X¹³X¹⁴,
wherein:
   each of X¹, X², X³, X⁴, X⁵, X⁶, X⁷, X⁸, X⁹, X¹⁰, X¹¹, X¹², X¹³, and X¹⁴ is independently an amino acid residue, wherein:
   X² comprises a side chain comprising an acidic or a polar group;
   X⁵ comprises a side chain comprising an acidic or a polar group;
   X¹³ comprises a side chain comprising an optionally substituted aromatic group; and
   two or more of X, X³, X⁴, X⁷, X¹⁰, X¹¹ and X¹⁴ are each independently an amino acid residue suitable for stapling, or are each independently stapled.
312. An agent, wherein the agent is or comprises:

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]ₚ₁₈[X¹⁹]ₚ₁₉[X²⁰]ₚ20[X²¹]ₚ₂₁[X²²]ₚ₂₂[X²³]ₚ₂₃,
wherein each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue.
313. An agent, wherein the agent is or comprises:

[X]ₚX₁X²X³X⁴X⁵X⁶X⁷X⁸X⁹X¹⁰X¹¹X¹X¹³X¹⁴[X¹⁵]ₚ₁₅[X¹⁶]ₚ₁₆[X¹⁷]ₚ₁₇[X]_{p′},
wherein:
   each of p15, p16 and p17 is independently 0 or 1;
   each of p and p′ is independently 0-10;
   each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.
314. An agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,
wherein:
   each of p0, p15, p16 and p17 is independently 0 or 1;
   each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
   X² comprises a side chain comprising an acidic or a polar group;
   X⁵ comprises a side chain comprising an acidic or a polar group;
   X¹³ comprises a side chain comprising an optionally substituted aromatic group; and
   two or more of X¹, X³, X⁴, X⁷, X¹⁰, X¹¹ and X¹⁴ are each independently an amino acid residue suitable for stapling, or are each independently stapled.
315. The agent of any one of the preceding Embodiments, the agent comprises three or more staples within 10-20 amino acid residues.
316. The agent of any one of the preceding Embodiments, the agent comprises three or more staples within 10-15 amino acid residues.
317. The agent of any one of the preceding Embodiments, the agent comprises three or more staples within 15 amino acid residues.
318. The agent of any one of the preceding Embodiments, the agent comprises three or more staples within 14 amino acid residues.
319. The agent of any one of the preceding Embodiments, the agent comprises three or more staples within 11 amino acid residues.
320. The agent of any one of the preceding Embodiments, wherein there are three staples in the peptide.
321. The agent of any one of Embodiments 1-319, wherein there are four staples in the peptide.
322. The agent of any one of the preceding Embodiments, wherein three or more of X⁰, X¹, X³, X⁴, X⁷, X¹⁰, X¹¹ and X¹⁴ are each independently an amino acid residue suitable for stapling, or are each independently stapled.
323. The agent of any one of the preceding Embodiments, wherein four or more of X⁰, X¹, X³, X⁴, X⁷, X¹⁰, X¹¹ and X¹⁴ are each independently an amino acid residue suitable for stapling, or are each independently stapled.
324. The agent of any one of the preceding Embodiments, wherein five of X⁰, X¹, X³, X⁴, X⁷, X¹⁰, X¹¹ and X¹⁴ are each independently an amino acid residue suitable for stapling, or are each independently stapled.
325. The agent of any one of the preceding Embodiments, wherein three or more of X¹, X³, X⁴, X⁷, X¹⁰, X¹¹ and X¹⁴ are each independently an amino acid residue suitable for stapling, or are each independently stapled.
326. The agent of any one of the preceding Embodiments, wherein four or more of X¹, X³, X⁴, X⁷, X¹⁰, X¹ and X¹⁴ are each independently an amino acid residue suitable for stapling, or are each independently stapled.
327. The agent of any one of the preceding Embodiments, wherein five of X¹, X³, X⁴, X⁷, X¹⁰, X¹¹ and X¹⁴ are each independently an amino acid residue suitable for stapling, or are each independently stapled.
328. The agent of any one of the preceding Embodiments, wherein X⁰ and X⁴ are each independently an amino acid residue suitable for stapling.
329. The agent of any one of Embodiments 1-327, wherein X⁰ and X⁴ are connected by a staple.
330. The agent of any one of the preceding Embodiments, wherein X¹ and X⁴ are each independently an amino acid residue suitable for stapling.
331. The agent of any one of Embodiments 1-327, wherein X¹ and X⁴ are connected by a staple.
332. The agent of any one of Embodiments 1-327, wherein X¹ and X³ are each independently an amino acid residue suitable for stapling.
333. The agent of any one of Embodiments 1-327, wherein X¹ and X³ are connected by a staple.
334. The agent of any one of the preceding Embodiments, wherein X⁴ and X¹¹ are each independently an amino acid residue suitable for stapling.
335. The agent of any one of Embodiments 1-333, wherein X⁴ and X¹¹ are connected by a staple.
336. The agent of Embodiment 309, wherein X¹, X⁴ and X¹¹ are each independently an amino acid residue suitable for stapling.
337. The agent of Embodiment 309, wherein X¹ and X⁴ are connected by a staple, and X⁴ and X¹¹ are connected by a staple.
338. The agent of any one of Embodiments 1-308, wherein the agent is an agent of any one of Embodiments 309-337.
339. The agent of any one of the preceding Embodiments, wherein X¹⁰ and X¹⁴ are each independently an amino acid residue suitable for stapling.
340. The agent of any one of Embodiments 1-337, wherein X¹⁰ and X¹⁴ are connected by a staple.
341. The agent of any one of the preceding Embodiments, wherein X⁷ and X¹⁰ are each independently an amino acid residue suitable for stapling.
342. The agent of any one of Embodiments 1-340, wherein X⁷ and X¹⁰ are connected by a staple.
343. The agent of any one of the preceding Embodiments, wherein X⁷ and X¹⁴ are each independently an amino acid residue suitable for stapling.
344. The agent of any one of Embodiments 1-342, wherein X⁷ and X¹⁴ are connected by a staple.
345. The agent of any one of Embodiments 1-331 and 334-340, wherein X³ and X⁷ are each independently an amino acid residue suitable for stapling.
346. The agent of any one of Embodiments 1-331 and 334-340, wherein X³ and X⁷ are connected by a staple.
347. The agent of any one of the preceding Embodiments, wherein the agent comprises a N-terminal group.
348. The agent of any one of the preceding Embodiments, wherein the N-terminal group is an acyl group.
349. The agent of Embodiment 347, wherein the N-terminal group comprises a moiety for stapling.
350. The agent of Embodiment 347, wherein the N-terminal group comprises a terminal olefin.
351. The agent of any one of the preceding Embodiments, wherein the agent comprises a N-terminal group which is Ac, NPyroR3, 5hexenyl, 4pentenyl, Bua, C3a, Cpc, Cbc, CypCO, Bnc, CF3CO, 2PyCypCO, 4THPCO, Isobutyryl, Ts, 15PyraPy, 2PyBu, 4PymCO, 4PyPrpc, 3IAPAc, 4MePipzPrpC, MePipAc, MeImid4SO2, BzAm20Allyl, Hex, 2PyzCO, 3Phc3, MeOPr, lithocholate, 2FPhc, PhC, MeSO2, Isovaleryl, EtHNCO, TzPyr, 8IAP, 3PydCO, 2PymCO, 5PymCO, 1Imidac, 2F2PyAc, 2IAPAc, 124TriPr, 6QuiAc, 3PyAc, 123TriAc, 1PyrazoleAc, 3PyPrpc, 5PymAc, 1PydoneAc, 124TriAc, Me2NAc, 8QuiSO2, mPEG4, mPEG8, mPEG16, or mPEG24.
352. The agent of Embodiment 347, wherein the N-terminal group is Ac.
353. The agent of any one of the preceding Embodiments, wherein X¹ is a residue of an amino acid having the structure of formula A-I, A-II or A-III, wherein R^{a1} and R^{a3} are taken together with their intervening atom(s) to form an optionally substituted 3-10 membered ring having 0-5 heteroatoms in addition to the intervening atom(s).
354. The agent of any one of the preceding Embodiments, wherein X¹ is —N(R^{a1})-L^{a1}-C(-L^{a}-R^{SP1})(R^{a3})-L^{a2}-C(O)—.
355. The agent of Embodiment 354, wherein R^{a1} is —H.
356. The agent of any one of Embodiments 354-355, wherein R^{a3} is —H.
357. The agent of any one of Embodiments 354-355, wherein R^{a3} is optionally substituted C₁₋₆ aliphatic.
358. The agent of any one of the preceding Embodiments, wherein X¹ is N(R^{a1})(-L^{a}-R^{SP1})-L^{a1}-C(R^{a2})(R^{a3})-L^{a2}-C(O)
359. The agent of Embodiment 358, wherein R^{a2} is —H.
360. The agent of Embodiment 358, wherein R^{a2} is optionally substituted C₁₋₆ aliphatic.
361. The agent of Embodiment 358, wherein R^{a2} is methyl.
362. The agent of Embodiments 354 and 358-361, wherein R^{a1} and R^{a3} are taken together with their intervening atom(s) to form an optionally substituted 3-10 membered ring having 0-3 heteroatoms in addition to the intervening atom(s).
363. The agent of Embodiment 362, wherein R^{a1} and R^{a3} are taken together with their intervening atom(s) to form an 5-membered saturated ring having no heteroatoms in addition to the nitrogen to which R^{a1} is attached.
364. The agent of any one of Embodiments 354-363, wherein L^{a1} is a covalent bond.
365. The agent of any one of Embodiments 354-364, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
366. The agent of any one of Embodiments 354-364, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
367. The agent of any one of Embodiments 354-364, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
368. The agent of any one of Embodiments 354-367, wherein L^{a} is optionally substituted —(CH₂)n- wherein n is 1, 2, 3, 4, 5, or 6.
369. The agent of any one of Embodiments 354-368, wherein L^{a} is —(CH₂)n- wherein n is 1, 2, 3, 4, 5, or 6.
370. The agent of any one of Embodiments 354-369, wherein L^{a2} is a covalent bond.
371. The agent of any one of Embodiments 354-370, wherein R^{SP1} is optionally substituted —CH═CH₂.
372. The agent of any one of Embodiments 354-370, wherein R^{SP1} is —CH═CH₂.
373. The agent of any one of Embodiments 354-370, wherein R^{SP1} is —COOH.
374. The agent of any one of Embodiments 354-370, wherein R^{SP1} is or comprises an amino group.
375. The agent of any one of Embodiments 354-370, wherein R^{SP1} is —NHR, wherein R is hydrogen or optionally substituted C₁₋₆ aliphatic.
376. The agent of any one of Embodiments 354-370, wherein R^{SP1} is —NHR, wherein R is C₁₋₆ alkyl.
377. The agent of any one of Embodiments 354-370, wherein R^{SP1} is —NH₂, wherein R is C₁₋₆ alkyl.
378. The agent of any one of Embodiments 354-370, wherein R^{SP1} is —N₃.
379. The agent of any one of Embodiments 354-370, wherein R^{SP1} is a terminal or activated alkyne.
380. The agent of any one of Embodiments 354-370, wherein R^{SP1} is —C≡CH.
381. The agent of any one of Embodiments 354-370, wherein R^{SP1} is —SH.
382. The agent of any one of Embodiments 1-352, wherein X¹ is PL3, S5, MePro, Asp, S6, Pro, Ala, Ser, ThioPro, Gly, NMebAla, TfeGA, or Asn.
383. The agent of any one of Embodiments 1-352, wherein X¹ is Ac-PL3, Ac-S5, NPyroR3-Asp, Ac-MePro, 5hexenyl-MePro, Ac-S6, 4pentenyl-MePro, Ac-Pro, Ac-Ala, Bua-PL3, C3a-PL3, Cpc-PL3, Cbc-PL3, CypCO-PL3, 4THPCO-PL3, Isobutyryl-PL3, Ac-Asp, Ac-Ser, Ts-PL3, 15PyraPy-PL3, 2PyBu-PL3, 4PymCO-PL3, 4pentenyl-ThioPro, 4PyPrpc-PL3, 3IAPAc-PL3, 4MePipzPrpC-PL3, MePipAc-PL3, MeImid4SO2-PL3, BzAm20Allyl-MePro, Ac-Gly, Ac-Sar, Ac-NMebAla, Hex-PL3, 2PyzCO-PL3, 3Phc3-PL3, MeOPr-PL3, lithocholate-PL3, 2FPhc-PL3, PhC-PL3, MeSO2-PL3, Isovaleryl-PL3, EtHNCO-PL3, TzPyr-PL3, 8IAP-PL3, 3PydCO-PL3, 2PymCO-PL3, 5PymCO-PL3, 1Imidac-PL3, 2F2PyAc-PL3, 2IAPAc-PL3, 124TriPr-PL3, 6QuiAc-PL3, 3PyAc-PL3, 123TriAc-PL3, 1PyrazoleAc-PL3, 3PyPrpc-PL3, 5PymAc-PL3, 1PydoneAc-PL3, 124TriAc-PL3, Me2NAc-PL3, 8QuiSO2-PL3, mPEG4-PL3, mPEG8-PL3, mPEG16-PL3, mPEG24-PL3, NPyroR3-Asn, or NPyroR3-Ser.
384. The agent of any one of Embodiments 1-352, wherein X¹ is PL3, [4pentenyl]MePro, [5hexenyl]MePro, or [BzAm20Allyl]MePro.
385. The agent of any one of Embodiments 1-352, wherein X¹ is PL3.
386. The agent of any one of Embodiments 1-352, wherein X¹ is [4pentenyl]MePro or [5hexenyl]MePro.
387. The agent of any one of the preceding Embodiments, wherein X¹ interacts with Val349 of beta-catenin or an amino acid residue corresponding thereto.
388. The agent of any one of the preceding Embodiments, wherein X³ is a residue of an amino acid that comprises a carboxyl group.
389. The agent of any one of the preceding Embodiments, wherein X³ is a residue of an amino acid having the structure of formula A-I, A-II or A-III, wherein R^{a2} or R^{a3} comprises a carboxyl group.
390. The agent of any one of the preceding Embodiments, wherein R^{a2} or R^{a3} is -L^{a}-CO₂R.
391. The agent of any one of the preceding Embodiments, wherein X³ is GlnR.
392. The agent of any one of Embodiments 1-387, wherein X³ is a residue of an amino acid that comprises an olefin.
393. The agent of any one of Embodiments 1-387 and 392, wherein X³ is a residue of an amino acid that comprises —CH═CH₂.
394. The agent of any one of Embodiments 1-387 and 392-393, wherein X³ is a residue of an amino acid that comprises —CH═CH₂ and forms a staple with another amino acid residue through olefin metathesis.
395. The agent of any one of Embodiments 1-387, wherein X³ is a residue of an amino acid having the structure of formula A-I, A-II or A-III, wherein R^{a2} or R^{a3} comprises an olefin.
396. The agent of any one of Embodiments 1-387 and 395, wherein X³ is a residue of an amino acid having the structure of formula A-I, A-II or A-III, wherein R^{a2} or R^{a3} is -L^{a}-CH═CH₂.
397. The agent of any one of the preceding Embodiments, wherein X³ is —N(R^{a1})-L^{a1}-C(-L^{a}-R^{SP1})(R^{a3})-L^{a2}-C(O)
398. The agent of Embodiment 397, wherein R^{a1} is —H.
399. The agent of any one of Embodiments 397-398, wherein R^{a3} is —H.
400. The agent of any one of Embodiments 397-398, wherein R^{a3} is optionally substituted C₁₋₆ aliphatic.
401. The agent of any one of Embodiments 397-400, wherein L^{a1} is a covalent bond.
402. The agent of any one of Embodiments 397-401, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
403. The agent of any one of Embodiments 397-401, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
404. The agent of any one of Embodiments 397-401, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
405. The agent of any one of Embodiments 397-402, wherein L^{a} is optionally substituted —(CH₂)n- wherein n is 1, 2, 3, 4, 5, or 6.
406. The agent of any one of Embodiments 397-402, wherein L^{a} is —(CH₂)n- wherein n is 1, 2, 3, 4, 5, or 6.
407. The agent of any one of Embodiments 397-406, wherein L^{a2} is a covalent bond.
408. The agent of any one of Embodiments 397-407, wherein R^{SP1} is optionally substituted —CH═CH₂.
409. The agent of any one of Embodiments 397-407, wherein R^{SP1} is —CH═CH₂.
410. The agent of any one of Embodiments 397-407, wherein R^{SP1} is —COOH.
411. The agent of any one of Embodiments 397-407, wherein R^{SP1} is or comprises an amino group.
412. The agent of any one of Embodiments 397-407, wherein R^{SP1} is —NHR, wherein R is hydrogen or optionally substituted C₁₋₆ aliphatic.
413. The agent of any one of Embodiments 397-407, wherein R^{SP1} is —NHR, wherein R is C₁₋₆ alkyl.
414. The agent of any one of Embodiments 397-407, wherein R^{SP1} is —NH₂.
415. The agent of any one of Embodiments 397-407, wherein R^{SP1} is —N₃.
416. The agent of any one of Embodiments 397-407, wherein R^{SP1} is a terminal or activated alkyne.
417. The agent of any one of Embodiments 397-407, wherein R^{SP1} is —C≡CH.
418. The agent of any one of Embodiments 397-407, wherein R^{SP1} is —SH.
419. The agent of any one of Embodiments 1-387 and 392-396, wherein X³ is AllylGly, [Bn][Allyl]Dap, [Phc][Allyl]Dap, [Piv][Allyl]Dap, or [CyCO][Allyl]Dap.
420. The agent of any one of the preceding Embodiments, wherein X⁴ is a residue of an amino acid that comprises an olefin.
421. The agent of any one of the preceding Embodiments, wherein X⁴ is a residue of an amino acid that comprises —CH═CH₂.
422. The agent of any one of the preceding Embodiments, wherein X⁴ is a residue of an amino acid that comprises —CH═CH₂ and forms a staple with another amino acid residue through olefin metathesis.
423. The agent of any one of the preceding Embodiments, wherein X⁴ is a residue of an amino acid having the structure of formula A-I, A-II or A-III, wherein R^{a2} or R^{a3} comprises an olefin.
424. The agent of any one of the preceding Embodiments, wherein X⁴ is a residue of an amino acid having the structure of formula A-I, A-II or A-III, wherein R^{a2} or R^{a3} is -L^{a}-CH═CH₂.
425. The agent of any one of the preceding Embodiments, wherein X⁴ is R⁵, R⁴, or R⁶.
426. The agent of any one of Embodiments 1-419, wherein X⁴ is a residue of an amino acid that comprises two olefins.
427. The agent of any one of Embodiments 1-419 and 426, wherein X⁴ is a residue of an amino acid that comprises two —CH═CH₂.
428. The agent of any one of Embodiments 1-419 and 426-427, wherein X⁴ is a residue of an amino acid that comprises two —CH═CH₂ and each forms a staple with another amino acid residue through olefin metathesis.
429. The agent of any one of Embodiments 1-419, wherein X⁴ is a residue of an amino acid having the structure of formula A-I, A-II or A-III, wherein R^{a2} and R^{a3} each independently comprises an olefin.
430. The agent of any one of Embodiments 1-419 and 429, wherein X⁴ is a residue of an amino acid having the structure of formula A-I, A-II or A-III, wherein R^{a2} and R^{a3} are each independently -L^{a}-CH═CH₂.
431. The agent of any one of the preceding Embodiments, wherein X⁴ is —N(R^{a1})-L^{a1}-C(-L^{a}-R^{SP1})(R³)-L^{a2}-C(O)—.
432. The agent of Embodiment 431, wherein R^{a1} is —H.
433. The agent of any one of Embodiments 431-432, wherein R^{a3} is —H.
434. The agent of any one of Embodiments 431-432, wherein R^{a3} is optionally substituted C₁₋₆ aliphatic.
435. The agent of any one of the preceding Embodiments, wherein X⁴ is —N(R^{a1})-L^{a}-C (-L^{a}-R_{SP1})(-L^{a}-R^{SP2})-L^{a2}-C(O).
436. The agent of any one of Embodiments 431-435, wherein Lai is a covalent bond.
437. The agent of any one of Embodiments 431-436, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
438. The agent of any one of Embodiments 431-436, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
439. The agent of any one of Embodiments 431-436, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
440. The agent of any one of Embodiments 431-437, wherein L^{a} bonded to R^{SP1} is optionally substituted —(CH₂)n- wherein n is 1, 2, 3, 4, 5, or 6.
441. The agent of any one of Embodiments 431-437, wherein L^{a} bonded to R^{SP1} is —(CH₂)n- wherein n is 1, 2, 3, 4, 5, or 6.
442. The agent of any one of Embodiments 431-441, wherein L^{a2} is a covalent bond.
443. The agent of any one of Embodiments 431-442, wherein R^{SP1} is optionally substituted —CH═CH₂.
444. The agent of any one of Embodiments 431-442, wherein R^{SP1} is —CH═CH₂.
445. The agent of any one of Embodiments 431-442, wherein R^{SP1} is —COOH.
446. The agent of any one of Embodiments 431-442, wherein R^{SP1} is or comprises an amino group.
447. The agent of any one of Embodiments 431-442, wherein R^{SP1} is —NHR, wherein R is hydrogen or optionally substituted C₁₋₆ aliphatic.
448. The agent of any one of Embodiments 431-442, wherein R^{SP1} is —NHR, wherein R is C₁₋₆ alkyl.
449. The agent of any one of Embodiments 431-442, wherein R^{SP1} is —NH₂.
450. The agent of any one of Embodiments 431-442, wherein R^{SP1} is —N₃.
451. The agent of any one of Embodiments 431-442, wherein R^{SP1} is a terminal or activated alkyne.
452. The agent of any one of Embodiments 431-442, wherein R^{SP1} is —C≡CH.
453. The agent of any one of Embodiments 431-442, wherein R^{SP1} is —SH.
454. The agent of any one of Embodiments 431-453, wherein R^{SP2} is optionally substituted —CH═CH₂.
455. The agent of any one of Embodiments 431-453, wherein R^{SP2} is —CH═CH₂.
456. The agent of any one of Embodiments 431-453, wherein R^{SP2} is —COOH.
457. The agent of any one of Embodiments 431-453, wherein R^{SP2} is or comprises an amino group.
458. The agent of any one of Embodiments 431-453, wherein R^{SP2} is —NHR, wherein R is hydrogen or optionally substituted C₁₋₆ aliphatic.
459. The agent of any one of Embodiments 431-453, wherein R^{SP2} is —NHR, wherein R is C₁₋₆ alkyl.
460. The agent of any one of Embodiments 431-453, wherein R^{SP2} is —NH₂.
461. The agent of any one of Embodiments 431-453, wherein R^{SP2} is —N₃.
462. The agent of any one of Embodiments 431-453, wherein R^{SP2} is a terminal or activated alkyne.
463. The agent of any one of Embodiments 431-453, wherein R^{SP2} is —C≡CH.
464. The agent of any one of Embodiments 431-453, wherein R^{SP2} is —SH.
465. The agent of any one of Embodiments 431-464, wherein L^{a} bonded to R^{SP1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
466. The agent of any one of Embodiments 431-464, wherein L^{a} bonded to R^{SP1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
467. The agent of any one of Embodiments 431-464, wherein L^{a} bonded to R^{SP1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
468. The agent of any one of Embodiments 431-464, wherein L^{a} bonded to R^{SP1} is optionally substituted —(CH₂)n- wherein n is 1, 2, 3, 4, 5, or 6.
469. The agent of any one of Embodiments 431-464, wherein L^{a} bonded to R^{SP1} is —(CH₂)n- wherein n is 1, 2, 3, 4, 5, or 6.
470. The agent of any one of Embodiments 435-469, wherein L^{a} bonded to R^{SP2} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
471. The agent of any one of Embodiments 435-469, wherein L^{a} bonded to R^{SP2} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
472. The agent of any one of Embodiments 435-469, wherein L^{a} bonded to R^{SP2} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
473. The agent of any one of Embodiments 435-469, wherein L^{a} bonded to R^{SP2} is optionally substituted —(CH₂)n- wherein n is 1, 2, 3, 4, 5, or 6.
474. The agent of any one of Embodiments 435-469, wherein L^{a} bonded to R^{SP2} is —(CH₂)n- wherein n is 1, 2, 3, 4, 5, or 6.
475. The agent of any one of Embodiments 1-419 and 426-430, wherein X⁴ is B5.
476. The agent of any one of Embodiments 1-419, wherein X⁴ is B5, Npg, Asp, R⁵, Ile, Ala, Cha, Chg, Ser, Leu, R⁴, R⁶, Phe, or S5.
477. The agent of any one of the preceding Embodiments, wherein X⁷ is a residue of an amino acid that comprises an optionally substituted carboxyl group, an optionally substituted amino group, or an azidyl group.
478. The agent of any one of the preceding Embodiments, wherein X⁷ is a residue of an amino acid having the structure of formula A-I, A-II or A-III, wherein R^{a2} or R^{a3} comprises a carboxyl group, an amino group, or an azidyl group.
479. The agent of any one of the preceding Embodiments, wherein X⁷ is a residue of an amino acid having the structure of formula A-I, A-II or A-III, wherein R^{a2} or R^{a3} is -L^{a}-CO₂R, -L^{a}-N₃, or -L^{a}-L-R.
480. The agent of any one of the preceding Embodiments, wherein X⁷ is —N(R^{a1})-L^{a1}-C(-L^{a}-R^{SP1})(R^{a3})-L^{a2}-C(O)—.
481. The agent of Embodiment 480, wherein R^{a1} is —H.
482. The agent of any one of Embodiments 480-481, wherein R^{a3} is —H
483. The agent of any one of Embodiments 480-481, wherein R^{a3} is optionally substituted C₁₋₆ aliphatic.
484. The agent of any one of Embodiments 480-483, wherein Lai is a covalent bond.
485. The agent of any one of Embodiments 480-484, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
486. The agent of any one of Embodiments 480-485, wherein L^{a} is optionally substituted —(CH₂)n- wherein n is 1, 2, 3, 4, 5, or 6.
487. The agent of any one of Embodiments 480-486, wherein L^{a} is —(CH₂)n- wherein n is 1, 2, 3, 4, 5, or 6.
488. The agent of any one of Embodiments 480-487, wherein L^{a2} is a covalent bond.
489. The agent of any one of Embodiments 480-488, wherein R^{SP1} is optionally substituted —CH═CH₂.
490. The agent of any one of Embodiments 480-489, wherein R^{SP1} is —CH═CH₂.
491. The agent of any one of Embodiments 480-488, wherein R^{SP1} is —COOH.
492. The agent of any one of Embodiments 480-488, wherein R^{SP1} is or comprises an amino group.
493. The agent of any one of Embodiments 480-488, wherein R^{SP1} is —NHR, wherein R is hydrogen or optionally substituted C₁₋₆ aliphatic.
494. The agent of any one of Embodiments 480-488, wherein R^{SP1} is —NHR, wherein R is C₁₋₆ alkyl.
495. The agent of any one of Embodiments 480-488, wherein R^{SP1} is —NH₂.
496. The agent of any one of Embodiments 480-488, wherein R^{SP1} is —N₃.
497. The agent of any one of Embodiments 480-488, wherein R^{SP1} is a terminal or activated alkyne.
498. The agent of any one of Embodiments 480-488, wherein R^{SP1} is —C≡CH.
499. The agent of any one of Embodiments 480-488, wherein R^{SP1} is —SH.
500. The agent of any one of the preceding Embodiments, wherein X⁷ is GlnR, Lys, [29N2spiroundecane]GlnR, [4aminopiperidine]GlnR, sAla, TriAzLys, [isophthalate]Lys, [succinate]Lys, [Me2Mal]Lys, [diphenate]Lys, or [Biphen33COOH]Lys.
501. The agent of any one of the preceding Embodiments, wherein X⁷ is GlnR, [29N2spiroundecane]GlnR, or [4aminopiperidine]GlnR.
502. The agent of any one of Embodiments 1-500, wherein X⁷ is Lys.
503. The agent of any one of Embodiments 1-500, wherein X⁷ is TriAzLys.
504. The agent of any one of the preceding Embodiments, wherein X¹⁰ is a residue of an amino acid that comprises an optionally substituted carboxyl group, an optionally substituted amino group, an azidyl group, an optionally substituted alkynyl group, or an optionally substituted thiol group.
505. The agent of any one of the preceding Embodiments, wherein X¹⁰ is a residue of an amino acid having the structure of formula A-I, A-II or A-III, wherein R^{a2} or R^{a3} comprises a carboxyl group, an amino group, an azidyl group, an alkynyl group, or a thiol group.
506. The agent of any one of the preceding Embodiments, wherein X¹⁰ is a residue of an amino acid having the structure of formula A-I, A-II or A-III, wherein R^{a2} or R^{a3} is -L^{a}-CO₂R, -L^{a}-N₃, or -L^{a}-L-R.
507. The agent of any one of the preceding Embodiments, wherein X¹⁰ is —N(R^{a1})-L^{a1}-C(-L^{a}-R_{SP1})(R^{a3})-L^{a2}-C(O)—.
508. The agent of Embodiment 507, wherein R^{a1} is —H.
509. The agent of any one of Embodiments 507-508, wherein R^{a3} is —H.
510. The agent of any one of Embodiments 507-508, wherein R^{a3} is optionally substituted C₁₋₆ aliphatic.
511. The agent of any one of Embodiments 507-510, wherein Lai is a covalent bond.
512. The agent of any one of Embodiments 507-511, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
513. The agent of any one of Embodiments 507-512, wherein L^{a} is optionally substituted —(CH₂)n- wherein n is 1, 2, 3, 4, 5, or 6.
514. The agent of any one of Embodiments 507-513, wherein L^{a} is —(CH₂)n- wherein n is 1, 2, 3, 4, 5, or 6.
515. The agent of any one of Embodiments 507-514, wherein L^{a2} is a covalent bond.
516. The agent of any one of Embodiments 507-515, wherein R^{SP1} is optionally substituted —CH═CH₂.
517. The agent of any one of Embodiments 507-515, wherein R^{SP1} is —CH═CH₂.
518. The agent of any one of Embodiments 507-515, wherein R^{SP1} is —COOH.
519. The agent of any one of Embodiments 507-515, wherein R^{SP1} is or comprises an amino group.
520. The agent of any one of Embodiments 507-515, wherein R^{SP1} is —NHR, wherein R is hydrogen or optionally substituted C₁₋₆ aliphatic.
521. The agent of any one of Embodiments 507-515, wherein R^{SP1} is —NHR, wherein R is C₁₋₆ alkyl.
522. The agent of any one of Embodiments 507-515, wherein R^{SP1} is —NH₂.
523. The agent of any one of Embodiments 507-515, wherein R^{SP1} is —N₃.
524. The agent of any one of Embodiments 507-515, wherein R^{SP1} is a terminal or activated alkyne.
525. The agent of any one of Embodiments 507-515, wherein R^{SP1} is —C≡CH.
526. The agent of any one of Embodiments 507-515, wherein R^{SP1} is —SH.
527. The agent of any one of the preceding Embodiments, wherein X¹⁰ is Lys, GlnR, TriAzLys, sAla, dLys, AsnR, hGlnR, iPrLys, TriAzOm, DGlnR, Orn, 4PipA, sCH2S, [8FBB]Cys, [4FB]Cys, [mXyl]Cys, [oXyl]Cys, [pXyl]Cys, dOrn, dDab, NMeOm, [2-6-naph]Cys, or [3-3-biph]Cys.
528. The agent of any one of the preceding Embodiments, wherein X¹⁰ is Lys, GlnR, or TriAzLys.
529. The agent of any one of Embodiments 1-528, wherein X¹⁰ is Lys.
530. The agent of any one of Embodiments 1-528, wherein X¹⁰ is GlnR.
531. The agent of any one of Embodiments 1-528, wherein X¹⁰ is TriAzLys.
532. The agent of any one of the preceding Embodiments, wherein X¹¹ is a residue of an amino acid that comprises an olefin.
533. The agent of any one of the preceding Embodiments, wherein X¹¹ is a residue of an amino acid that comprises —CH═CH₂.
534. The agent of any one of the preceding Embodiments, wherein X¹¹ is a residue of an amino acid that comprises —CH═CH₂ and forms a staple with another amino acid residue through olefin metathesis.
535. The agent of any one of the preceding Embodiments, wherein X¹¹ is a residue of an amino acid having the structure of formula A-I, A-II or A-III, wherein R^{a2} or R^{a3} comprises an olefin.
536. The agent of any one of the preceding Embodiments, wherein X¹¹ is a residue of an amino acid having the structure of formula A-I, A-II or A-III, wherein R^{a2} or R^{a3} is -L^{a}-CH═CH₂.
537. The agent of any one of the preceding Embodiments, wherein X¹¹ is —N(R^{a1})-L^{a1}-C(-L^{a}-R^{SP1})(R^{a3})-L^{a2}-C(O)—.
538. The agent of Embodiment 537, wherein R^{a1} is —H.
539. The agent of any one of Embodiments 537-538, wherein L^{a1} is a covalent bond.
540. The agent of any one of Embodiments 537-539, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
541. The agent of any one of Embodiments 537-539, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
542. The agent of any one of Embodiments 537-539, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
543. The agent of any one of Embodiments 537-540, wherein L^{a} is —(CH₂)n- wherein n is 1, 2, 3, 4, 5, or 6.
544. The agent of any one of Embodiments 537-540, wherein L^{a2} is a covalent bond.
545. The agent of any one of Embodiments 537-544, wherein R^{SP1} is optionally substituted —CH═CH₂.
546. The agent of any one of Embodiments 537-544, wherein R^{SP1} is —CH═CH₂.
547. The agent of any one of Embodiments 537-544, wherein R^{SP1} is —COOH.
548. The agent of any one of Embodiments 537-544, wherein R^{SP1} is or comprises an amino group.
549. The agent of any one of Embodiments 537-544, wherein R^{SP1} is —NHR, wherein R is hydrogen or optionally substituted C₁₋₆ aliphatic.
550. The agent of any one of Embodiments 537-544, wherein R^{SP1} is —NHR, wherein R is C₁₋₆ alkyl.
551. The agent of any one of Embodiments 537-544, wherein R^{SP1} is —NH₂.
552. The agent of any one of Embodiments 537-544, wherein R^{SP1} is —N₃.
553. The agent of any one of Embodiments 537-544, wherein R^{SP1} is a terminal or activated alkyne.
554. The agent of any one of Embodiments 537-544, wherein R^{SP1} is —C≡CH.
555. The agent of any one of Embodiments 537-544, wherein R^{SP1} is —SH.
556. The agent of any one of Embodiments 537-555, wherein one methylene unit of L is replaced with —N(R′)—.
557. The agent of any one of Embodiments 537-555, wherein one methylene unit of L is replaced with —N(R′)C(O)O—.
558. The agent of any one of Embodiments 556-557, wherein R′ is —H.
559. The agent of any one of Embodiments 556-557, wherein R′ is C₁₋₆ aliphatic.
560. The agent of any one of Embodiments 556-557, wherein R′ and R^{a3} are taken together with their intervening atom(s) to form an optionally substituted 3-14 membered ring having 0-5 heteroatoms in addition to the nitrogen atom to which R′ is attached.
561. The agent of any one of Embodiments 556-557, wherein R′ and R^{a3} are taken together with their intervening atom(s) to form an optionally substituted 3-8 membered ring having 0-5 heteroatoms in addition to the nitrogen atom to which R′ is attached.
562. The agent of any one of Embodiments 556-557, wherein R′ and R^{a3} are taken together with their intervening atom(s) to form an optionally substituted 3-7 membered ring having no heteroatoms in addition to the nitrogen atom to which R′ is attached.
563. The agent of any one of Embodiments 560-562, wherein the ring is monocyclic.
564. The agent of any one of Embodiments 560-563, wherein the ring is saturated.
565. The agent of any one of Embodiments 560-564, wherein the ring is 5-membered.
566. The agent of any one of Embodiments 537-559, wherein R^{a3} is —H.
567. The agent of any one of Embodiments 537-559, wherein R^{a3} is optionally substituted C₁₋₆ aliphatic.
568. The agent of any one of Embodiments 537-555 and 566-567, wherein L^{a} is optionally substituted —(CH₂)n- wherein n is 1, 2, 3, 4, 5, or 6.
569. The agent of Embodiment 568, wherein L^{a} is —(CH₂)n- wherein n is 1, 2, 3, 4, 5, or 6.
570. The agent of any one of the preceding Embodiments, wherein X¹¹ is PyrS2, Lys, 3Thi, Ala, Phe, SPip3, PyrSadNip3Butene, SPip2, Az3, DapAc7EDA, Leu, 3allyloxyPyrSa, PyrSaV3Butene, Az2, PyrS1, PyrSc72SMe3ROMe, PyrSc72RMe3SOMe, PyrSc7045RMe, PyrSc7045SMe, PyrSc73Me2, PyrSc7, PyrSaA3Butene, PyrSadA3Butene, Dap7Gly, Dap7Pent, DapAc7PDA, Dap7Abu, 4VinylPyrSa, PyrSadV3Butene, PyrSaSar3Butene, PyrSaNip3Butene, PyrSaPro3Butene, PyrSa4VinMe2PhAc, or 3allylPyrSa.
571. The agent of any one of the preceding Embodiments, wherein X¹¹ is PyrS2.
572. The agent of any one of the preceding Embodiments, wherein X¹⁴ is a residue of an amino acid that comprises a carboxyl group, an amino group, an azidyl group, an alkynyl group, or a thiol group.
573. The agent of any one of the preceding Embodiments, wherein X¹⁴ is a residue of an amino acid having the structure of formula A-I, A-II or A-III, wherein R^{a2} or R^{a3} comprises a carboxyl group, an amino group, an azidyl group, an alkynyl group, or a thiol group.
574. The agent of any one of the preceding Embodiments, wherein X⁴ is —N(R^{a1})-L^{a1}-C(-L^{a}-R^{SP1})(R^{a3})-L^{a2}-C(O)—.
575. The agent of Embodiment 574, wherein R^{a1} is —H.
576. The agent of Embodiment 574-575, wherein R^{a3} is —H.
577. The agent of Embodiment 574-575, wherein R^{a3} is optionally substituted C₁₋₆ aliphatic.
578. The agent of Embodiment 574-577, wherein Lai is a covalent bond.
579. The agent of Embodiment 574-578, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
580. The agent of Embodiment 574-578, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
581. The agent of Embodiment 574-578, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
582. The agent of Embodiment 574-579, wherein L^{a} is optionally substituted —(CH₂)n- wherein n is 1, 2, 3, 4, 5, or 6.
583. The agent of Embodiment 574-579, wherein L^{a} is —(CH₂)n- wherein n is 1, 2, 3, 4, 5, or 6.
584. The agent of Embodiment 574-583, wherein L^{a2} is a covalent bond.
585. The agent of Embodiment 574-584, wherein R^{SP1} is optionally substituted —CH═CH₂.
586. The agent of Embodiment 574-584, wherein R^{SP1} is —CH═CH₂.
587. The agent of Embodiment 574-584, wherein R^{SP1} is —COOH.
588. The agent of Embodiment 574-584, wherein R^{SP1} is or comprises an amino group.
589. The agent of Embodiment 574-584, wherein R^{SP1} is —NHR, wherein R is hydrogen or optionally substituted C₁₋₆ aliphatic.
590. The agent of Embodiment 574-584, wherein R^{SP1} is —NHR, wherein R is C₁₋₆ alkyl.
591. The agent of Embodiment 574-584, wherein R^{SP1} is —NH₂.
592. The agent of Embodiment 574-584, wherein R^{SP1} is —N₃.
593. The agent of Embodiment 574-584, wherein R^{SP1} is a terminal or activated alkyne.
594. The agent of Embodiment 574-584, wherein R^{SP1} is —C≡CH.
595. The agent of Embodiment 574-584, wherein R^{SP1} is —SH.
596. The agent of any one of the preceding Embodiments, wherein X¹⁴ is a residue of an amino acid having the structure of formula A-I, A-II or A-III, wherein R^{a2} or R^{a3} is -L^{a}-CO₂R, -L^{a}-N₃, or -L^{a}-L-R.
597. The agent of any one of the preceding Embodiments, wherein X¹⁴ is GlnR, Lys, sAla, Gln, Cys, TriAzLys, AsnR, hGlnR, 4PipA, sAbu, Orn, GlnR, [4mampiperidine]GlnR, [39N2spiroundecane]GlnR, [29N2spiroundecane]GlnR, iPrLys, sCH2S, [diaminobutane]GlnR, [4aminopiperidine]GlnR, dGlnR.
598. The agent of any one of Embodiments 1-597, wherein X¹⁴ is GlnR, Lys, or sAla.
599. The agent of any one of Embodiments 1-598, wherein X¹⁴ is GlnR.
600. The agent of any one of Embodiments 1-598, wherein X¹⁴ is Lys.
601. The agent of any one of Embodiments 1-598, wherein X¹⁴ is sAla.
602. The agent of any one of the preceding Embodiments, wherein a pair of amino acid residues suitable for stapling each independently comprises an acid group.
603. The agent of any one of the preceding Embodiments, wherein a pair of amino acid residues suitable for stapling each independently comprises —COOH or an activated form thereof.
604. The agent of any one of Embodiments 602-603, wherein the pair is stapled by reacting with a linking reagent which is a diamine or a salt thereof.
605. The agent of any one of the preceding Embodiments, wherein a pair of amino acid residues suitable for stapling each independently comprises an amino group.
606. The agent of Embodiment 605, wherein the pair is stapled by reacting with a linking reagent which is a di-acid or a salt thereof.
607. The agent of Embodiment 605, wherein the pair is stapled by reacting with a linking reagent comprising two —COOH or a salt thereof.
608. The agent of any one of the preceding Embodiments, wherein a pair of amino acid residues suitable for stapling each independently comprises a reactive group, and the reactive group of one can react with the other through a cycloaddition reaction.
609. The agent of any one of the preceding Embodiments, wherein one of a pair of amino acid residues suitable for stapling comprises —N₃ and the other comprises an alkyne.
610. The agent of Embodiment 609, wherein the pair is stapled through a click reaction.
611. The agent of any one of the preceding Embodiments, wherein a pair of amino acid residues suitable for stapling each independently comprises a nucleophilic group.
612. The agent of any one of the preceding Embodiments, wherein a pair of amino acid residues suitable for stapling each independently comprises —SH.
613. The agent of any one of Embodiments 611-612, wherein the pair is stapled by reacting a linking reagent comprising two leaving groups.
614. The agent of any one of Embodiments 611-613, wherein the pair is stapled by reacting a linking reagent having the structure of R^{x}-L″-R^{x}, wherein each R^{x} is independently a leaving group.
615. The agent of any one of Embodiments 613-614, wherein each leaving group is —Br.
616. The agent of any one of the preceding Embodiments, wherein one of X¹⁰ and X¹⁴ is a residue of an amino acid that comprises a carboxyl group, and the other is a residue of an amino acid that comprises an amino group.
617. The agent of any one of the preceding Embodiments, wherein X¹⁰ and X¹⁴ are connected by a staple, wherein the staple comprises —C(O)N(R′)—.
618. The agent of any one of the preceding Embodiments, wherein one of X⁷ and X¹⁰ is a residue of an amino acid that comprises a carboxyl group, and the other is a residue of an amino acid that comprises an amino group.
619. The agent of any one of the preceding Embodiments, wherein X⁷ and X¹⁰ are connected by a staple, wherein the staple comprises —C(O)N(R′)—.
620. The agent of any one of the preceding Embodiments, wherein one of X⁷ and X¹⁴ is a residue of an amino acid that comprises a carboxyl group, and the other is a residue of an amino acid that comprises an amino group.
621. The agent of any one of the preceding Embodiments, wherein X⁷ and X¹⁴ are connected by a staple, wherein the staple comprises —C(O)N(R′)—.
622. The agent of any one of the preceding Embodiments, wherein one of X³ and X⁷ is a residue of an amino acid that comprises a carboxyl group, and the other is a residue of an amino acid that comprises an amino group.
623. The agent of any one of the preceding Embodiments, wherein X³ and X⁷ are connected by a staple, wherein the staple comprises —C(O)N(R′)—.
624. The agent of any one of the preceding Embodiments, wherein one of X¹⁰ and X¹⁴ is a residue of an amino acid that comprises an azidyl group, and the other is a residue of an amino acid that comprises an alkynyl group.
625. The agent of any one of the preceding Embodiments, wherein one of X¹⁰ and X¹⁴ are connected by a staple, wherein the staple comprises an optionally substituted triazolylene ring.
626. The agent of any one of the preceding Embodiments, wherein one of X⁷ and X¹⁰ is a residue of an amino acid that comprises an azidyl group, and the other is a residue of an amino acid that comprises an alkynyl group.
627. The agent of any one of the preceding Embodiments, wherein one of X⁷ and X¹⁰ are connected by a staple, wherein the staple comprises an optionally substituted triazolylene ring.
628. The agent of any one of the preceding Embodiments, wherein X¹⁰ and X¹⁴ are residues of amino acids that each independently comprises a thiol group.
629. The agent of any one of the preceding Embodiments, wherein X¹⁰ and X¹⁴ are connected by a staple, wherein the staple comprises —S-Cy-S—.
630. An agent, which is a stapled peptide comprising three staples, wherein the first and second staples are bonded to the same amino acid residue, and the third staple are bonded to two amino acid residues none of which is bonded to the first or second staple.
631. An agent, which is a stapled peptide comprising three staples, wherein the first and second staples are bonded to the same amino acid residue, and the third staple are bonded to two amino acid residues none of which is bonded to the first or second staple.
632. The agent of any one of the preceding Embodiments, comprising a staple having the structure of L^{s} which is -L^{s1}-L^{s2}-L^{s3}-.
633. The agent of any one of the preceding Embodiments, comprising three staples each independently having the structure of L^{s} which is -L^{s1}-L^{s2}-L^{s3}-.
634. The agent of any one of the preceding Embodiments, wherein there are three staples in the agent each independently having the structure of L^{s} which is -L^{s1}-L^{s2}-L^{s3}-.
635. The agent of any one of Embodiments 1-632, comprising four staples each independently having the structure of L^{s} which is -L^{s1}-L^{s2}-L^{s3}-.
636. The agent of any one of Embodiments 1-632, wherein there are four staples in the agent each independently having the structure of L^{s} which is -L^{s1}-L^{s2}-L^{s3}-.
637. The agent of any one of the preceding Embodiments, comprising a staple having the structure of L^{s} which is -L^{s1}-L^{s2}-L^{s3}- wherein the staple is bonded to X¹ and X³.
638. The agent of any one of the preceding Embodiments, comprising a staple having the structure of L^{s} which is -L^{s1}-L^{s2}-L^{s3}- wherein the staple is bonded to X¹ and X⁴.
639. The agent of any one of the preceding Embodiments, comprising a staple having the structure of L^{s} which is -L^{s1}-L^{s2}-L^{s3}- wherein the staple is bonded to X⁴ and X¹¹.
640. The agent of any one of the preceding Embodiments, comprising a staple having the structure of L^{s} which is -L^{s1}-L^{s2}-L^{s3}- wherein the staple is bonded to X³ and X⁷.
641. The agent of any one of the preceding Embodiments, comprising a staple having the structure of L^{s} which is -L^{s1}-L^{s2}-L^{s3}- wherein the staple is bonded to X⁷ and X¹⁰.
642. The agent of any one of the preceding Embodiments, comprising a staple having the structure of L^{s} which is -L^{s1}-L^{s2}-L^{s3}- wherein the staple is bonded to X⁷ and X¹⁴.
643. The agent of any one of the preceding Embodiments, comprising a staple having the structure of L^{s} which is -L^{s1}-L^{s2}-L^{s3}- wherein the staple is bonded to X¹⁰ and X¹⁴.
644. The agent of any one of Embodiments 632-643, wherein L^{s1} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
645. The agent of Embodiment 644, wherein L^{s1} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
646. The agent of Embodiment 644, wherein L^{s1} is an optionally substituted bivalent linear or branched, saturated or partially unsaturated C₁₋₆ hydrocarbon chain, wherein one or more methylene units are optionally and independently replaced with —O—, -Cy-, —S—, —N(R′)—, —C(O)—, —C(O)N(R′)—, or —N(R′)C(O)O—.
647. The agent of Embodiment 644, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
648. The agent of any one of Embodiments 645-647, wherein L^{s1} comprises —N(R′)—.
649. The agent of any one of Embodiments 645-647, wherein L^{s1} comprises —N(R′)C(O)O—.
650. The agent of Embodiment 649, wherein —N(R′)— is closer to L^{s2}.
651. The agent of Embodiment 649, wherein —O— is closer to L^{s2}.
652. The agent of any one of Embodiments 645-647, wherein L^{s1} is —(CH₂)m-N(R′)—(CH₂)n-, wherein each of m and n is independently 1, 2, 3, 4, 5, 6, 7, 8, 9, or 10.
653. The agent of any one of Embodiments 645-647, wherein L^{s1} is —(CH₂)m-N(R′)—C(O)—O—(CH₂)n-, wherein each of m and n is independently 1, 2, 3, 4, 5, 6, 7, 8, 9, or 10.
654. The agent of any one of Embodiments 652-653, wherein —(CH₂)m- is bonded L^{s2}.
655. The agent of any one of Embodiments 652-653, wherein —(CH₂)n- is bonded L^{s2}.
656. The agent of any one of Embodiments 652-655, wherein m is 1.
657. The agent of any one of Embodiments 652-655, wherein m is 2.
658. The agent of any one of Embodiments 652-657, wherein n is 3.
659. The agent of any one of Embodiments 648-658, wherein R′ is —H.
660. The agent of any one of Embodiments 648-658, wherein R′ is optionally substituted C₁₋₆ aliphatic.
661. The agent of any one of Embodiments 648-658, wherein R′ is methyl.
662. The agent of any one of Embodiments 648-658, wherein R′ is taken together with R^{a3} of the amino acid residue to which L^{s1} is bonded to and their intervening atom(s) to form an optionally substituted 3-10 membered ring having 0-5 heteroatoms in addition to the intervening atom(s).
663. The agent of Embodiment 662, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
664. The agent of any one of Embodiments 662-663, wherein the formed ring is saturated.
665. The agent of any one of Embodiments 662-664, wherein the formed ring is 4-membered.
666. The agent of any one of Embodiments 662-664, wherein the formed ring is 5-membered.
667. The agent of any one of Embodiments 662-666, wherein the formed ring has no heteroatoms in addition to the intervening atom(s).
668. The agent of Embodiment 644, wherein L^{s1} is optionally substituted —(CH₂)n-, wherein n is 1, 2, 3, 4, 5, or 6.
669. The agent of Embodiment 644, wherein L^{s1} is —(CH₂)n-, wherein n is 1, 2, 3, 4, 5, or 6.
670. The agent of Embodiment 644, wherein L^{s1} is —CH₂—.
671. The agent of Embodiment 644, wherein L^{s1} is optionally substituted —(CH₂)n-C(O)—, wherein n is 1, 2, 3, 4, 5, or 6.
672. The agent of Embodiment 644, wherein L^{s1} is —(CH₂)n-C(O)—, wherein n is 1, 2, 3, 4, 5, or 6.
673. The agent of Embodiment 644, wherein L^{s1} is —(CH₂)n-C(O)—, wherein n is 2 or 3.
674. The agent of any one of Embodiments 632-673, wherein L^{s1} is bonded to an amino acid residue closer to the N-terminus than an amino acid residue to which -L^{s3}- is bond.
675. The agent of any one of Embodiments 632-674, wherein L^{s1} is bond to a carbon atom of the peptide backbone.
676. The agent of any one of Embodiments 632-675, wherein L^{s1} is bond to an alpha carbon atom of an amino acid residue.
677. The agent of any one of Embodiments 632-674, wherein L^{s1} is bond to a nitrogen atom of the peptide backbone.
678. The agent of any one of Embodiments 632-674, wherein L^{s1} is bond to a nitrogen atom of the peptide backbone, wherein the nitrogen atom is of an amino group bonded to an alpha carbon atom of an amino acid residue.
679. The agent of any one of Embodiments 677-678, wherein the nitrogen atom is bond to —C(O)— of L^{s}.
680. The agent of any one of Embodiments 632-679, wherein L^{s2} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
681. The agent of any one of Embodiments 632-679, wherein L^{s2} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
682. The agent of Embodiment 681, wherein L^{s2} is optionally substituted —CH═CH—.
683. The agent of Embodiment 681, wherein L^{s2} is —CH═CH—.
684. The agent of Embodiment 681, wherein the double bond is E.
685. The agent of Embodiment 681, wherein the double bond is Z.
686. The agent of Embodiment 681, wherein L^{s2} is optionally substituted —CH₂—CH₂—.
687. The agent of Embodiment 681, wherein L^{s2} is —CH₂—CH₂—.
688. The agent of Embodiment 681, wherein L^{s2} is -Cy-.
689. The agent of Embodiment 688, wherein -Cy- is optionally substituted saturated or partially unsaturated 5-6 membered ring having 0-4 heteroatoms.
690. The agent of Embodiment 688, wherein -Cy- is optionally substituted phenyl ring.
691. The agent of Embodiment 688, wherein -Cy- is optionally substituted 5-6 membered aromatic ring having 1-4 heteroatoms.
692. The agent of Embodiment 688, wherein -Cy- is optionally substituted

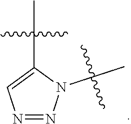 [SMILES: *c1cnnn1*]

693. The agent of Embodiment 688, wherein -Cy- is

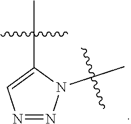 [SMILES: *c1cnnn1*]

694. the agent of Embodiment 688, wherein -Cy- is optionally substituted

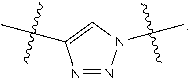 [SMILES: *c1cn(*)nn1]

695. The agent of Embodiment 688, wherein -Cy- is

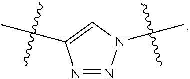 [SMILES: *c1cn(*)nn1]

696. The agent of any one of Embodiments 694-695, wherein the carbon atom is bonded to L^{s1}.
697. The agent of any one of Embodiments 694-695, wherein the carbon atom is bonded to L^{s3}.
698. The agent of Embodiment 681, wherein L^{s2} is —C(O)N(R′)—.
699. The agent of Embodiment 698, wherein R′ is —H.
700. The agent of Embodiment 698, wherein R′ is optionally substituted C₁₋₆ aliphatic.
701. The agent of any one of Embodiments 698-700, wherein the —N(R′)— is bonded to L^{s1}.
702. The agent of any one of Embodiments 698-700, wherein the —N(R′)— is bonded to L^{s3}.
703. The agent of Embodiment 681, wherein one or more methylene units are independently replaced with —C(O)N(R′)— or —N(R′)—, and one or more methylene units are independently replaced with —C(R′)₂—, wherein one or more R′ of one or more —C(R′)₂— are each independently taken together with R′ of —C(O)N(R′)— or —N(R′)— and their intervening atom(s) to form an optionally substituted 3-10 membered ring having 0-5 heteroatoms in addition to the intervening atom(s).
704. The agent of Embodiment 703, wherein the formed ring is saturated.
705. The agent of any one of Embodiments 703-704, wherein the formed ring is 4-membered.
706. The agent of any one of Embodiments 703-705, wherein the formed ring is 5-membered.
707. The agent of any one of Embodiments 703-706, wherein the formed ring has no heteroatoms in addition to the intervening atom(s).
708. The agent of Embodiment 681, wherein L^{s2} is —S-L″-S—.
709. The agent of Embodiment 708, wherein L^{s1} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
710. The agent of Embodiment 708, wherein L^{s1} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
711. The agent of Embodiment 708, wherein L^{s1} is or comprise -Cy-.
712. The agent of Embodiment 708, wherein L^{s1} is or comprise —(CH₂)m-Cy-(CH₂)n-, wherein each m and n is optionally substituted 0 1, 2, 3, 4, 5, 6, 7, 8, 9, or 10, and each —CH₂— is optionally substituted.
713. The agent of Embodiment 712, wherein each m and n is independently 1.
714. The agent of any one of Embodiments 711-713, wherein is optionally substituted phenyl.
715. The agent of any one of Embodiments 711-713, wherein is optionally substituted 5-6 membered aromatic ring having 1-4 heteroatoms.
716. The agent of any one of Embodiments 632-715, wherein L^{s3} is a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
717. The agent of Embodiment 716, wherein L^{s3} is an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
718. The agent of Embodiment 716, wherein L^{s3} is an optionally substituted bivalent linear or branched, saturated or partially unsaturated C₁₋₆ hydrocarbon chain, wherein one or more methylene units are optionally and independently replaced with —O—, -Cy-, —S—, —N(R′)—, —C(O)—, —C(O)N(R′)—, or —N(R′)C(O)O—.
719. The agent of Embodiment 716, wherein L^{s3} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
720. The agent of any one of Embodiments 717-719, wherein L^{s3} comprises —N(R′)—.
721. The agent of any one of Embodiments 717-719, wherein L^{s3} comprises —N(R′)C(O)O—.
722. The agent of Embodiment 721, wherein —N(R′)— is closer to L^{s2}.
723. The agent of Embodiment 721, wherein —O— is closer to L^{s2}.
724. The agent of any one of Embodiments 717-719, wherein L^{s3} is —(CH₂)m-N(R′)—(CH₂)n-, wherein each of m and n is independently 1, 2, 3, 4, 5, 6, 7, 8, 9, or 10.
725. The agent of any one of Embodiments 717-719, wherein L^{s3} is —(CH₂)m-N(R′)—C(O)—O—(CH₂)n-, wherein each of m and n is independently 1, 2, 3, 4, 5, 6, 7, 8, 9, or 10.
726. The agent of any one of Embodiments 724-725, wherein —(CH₂)n- is bonded L^{s2}.
727. The agent of any one of Embodiments 724-725, wherein —(CH₂)m- is bonded L^{s2}.
728. The agent of any one of Embodiments 724-727, wherein m is 1.
729. The agent of any one of Embodiments 724-727, wherein m is 2.
730. The agent of any one of Embodiments 724-729, wherein n is 3.
731. The agent of any one of Embodiments 720-730, wherein R′ is —H.
732. The agent of any one of Embodiments 720-730, wherein R′ is optionally substituted C₁₋₆ aliphatic.
733. The agent of any one of Embodiments 720-730, wherein R′ is methyl.
734. The agent of any one of Embodiments 720-730, wherein R′ is taken together with R^{a3} of the amino acid residue to which L^{s3} is bonded to and their intervening atom(s) to form an optionally substituted 3-10 membered ring having 0-5 heteroatoms in addition to the intervening atom(s).
735. The agent of any one of Embodiments 720-730, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
736. The agent of any one of Embodiments 734-735, wherein the formed ring is saturated.
737. The agent of any one of Embodiments 734-736, wherein the formed ring is 4-membered.
738. The agent of any one of Embodiments 734-737, wherein the formed ring is 5-membered.
739. The agent of any one of Embodiments 734-738, wherein the formed ring has no heteroatoms in addition to the intervening atom(s).
740. The agent of Embodiment 716, wherein L^{s3} is optionally substituted —(CH₂)n-, wherein n is 1, 2, 3, 4, 5, or 6.
741. The agent of Embodiment 716, wherein L^{s3} is —(CH₂)n-, wherein n is 1, 2, 3, 4, 5, or 6.
742. The agent of Embodiment 716, wherein L^{s3} is —(CH₂)₃—.
743. The agent of Embodiment 716, wherein L^{s3} is —(CH₂)₂—.
744. The agent of Embodiment 716, wherein L^{s3} is —CH₂—.
745. The agent of Embodiment 716, wherein L^{s3} is optionally substituted —(CH₂)n-C(O)—, wherein n is 1, 2, 3, 4, 5, or 6.
746. The agent of Embodiment 716, wherein L^{s3} is —(CH₂)n-C(O)—, wherein n is 1, 2, 3, 4, 5, or 6.
747. The agent of Embodiment 716, wherein L^{s3} is —(CH₂)n-C(O)—, wherein n is 2 or 3.
748. The agent of any one of Embodiments 632-747, wherein L^{s3} is bonded to an amino acid residue closer to the N-terminus than an amino acid residue to which -L^{s3}- is bond.
749. The agent of any one of Embodiments 632-748, wherein L^{s3} is bond to a carbon atom of the peptide backbone.
750. The agent of any one of Embodiments 632-749, wherein L^{s3} is bond to an alpha carbon atom of an amino acid residue.
751. The agent of any one of Embodiments 632-748, wherein L^{s3} is bond to a nitrogen atom of the peptide backbone.
752. The agent of any one of Embodiments 632-748, wherein L^{s3} is bond to a nitrogen atom of the peptide backbone, wherein the nitrogen atom is of an amino group bonded to an alpha carbon atom of an amino acid residue.
753. The agent of any one of Embodiments 751-752, wherein the nitrogen atom is bond to —C(O)— of L^{s3}.
754. The agent of any one of Embodiments 632-753, wherein a staple is optionally substituted —CH₂—CH═CH—(CH₂)₃—.
755. The agent of any one of Embodiments 632-753, wherein a staple is —CH₂—CH═CH—(CH₂)₃—.
756. The agent of Embodiment 754-755, wherein —CH═CH— is E.
757. The agent of Embodiment 754-755, wherein —CH═CH— is Z.
758. The agent of any one of Embodiments 632-753, wherein a staple is optionally substituted —CH₂—CH═CH—(CH₂)₃—C(O)—.
759. The agent of any one of Embodiments 632-753, wherein a staple is —CH₂—CH═CH—(CH₂)₃—C(O)—.
760. The agent of any one of Embodiments 632-753, wherein a staple is optionally substituted —CH₂—CH═CH—(CH₂)₂—C(O)—.
761. The agent of any one of Embodiments 632-753, wherein a staple is —CH₂—CH═CH—(CH₂)₂—C(O)—.
762. The agent of Embodiment 758-761, wherein —CH═CH— is E.
763. The agent of Embodiment 758-761, wherein —CH═CH— is Z.
764. The agent of any one of Embodiments 632-753, wherein a staple is optionally substituted —(CH₂)n-, wherein n is 1-20.
765. The agent of any one of Embodiments 632-753, wherein a staple is —(CH₂)n-, wherein n is 1-20.
766. The agent of any one of Embodiments 632-753, wherein a staple is optionally substituted —(CH₂)n-CO—, wherein n is 1-20.
767. The agent of any one of Embodiments 632-753, wherein a staple is —(CH₂)n-C(O)—, wherein n is 1-20.
768. The agent of Embodiment 764-767, wherein n is 4-10.
769. The agent of Embodiment 764-767, wherein n is 5-8.
770. The agent of Embodiment 764-767, wherein n is 6.
771. The agent of any one of Embodiments 754-770, wherein optionally substituted —(CH₂)₃- or —C(O)— is bonded to an amino acid residue closer to a N-terminus to the other amino acid residue bonded to the same staple.
772. The agent of any one of Embodiments 754-771, wherein optionally substituted —(CH₂)₃- or —C(O)— is bonded to an alpha-carbon atom of an amino acid residue.
773. The agent of any one of Embodiments 754-771, wherein optionally substituted —(CH₂)₃- or —C(O)— is bonded to a nitrogen atom of an amino acid residue.
774. The agent of any one of Embodiments 754-771, wherein optionally substituted —(CH₂)₃- or —C(O)— is bonded to a nitrogen atom bonded to an alpha carbon atom of an amino acid residue.
775. The agent of any one of Embodiments 754-774, wherein optionally substituted —(CH₂)₃- or —C(O)— is bonded to X¹.
776. The agent of Embodiment 775, wherein the other amino acid residue bonded to the staple is X³.
777. The agent of Embodiment 775, wherein the other amino acid residue bonded to the staple is X⁴.
778. The agent of any one of Embodiments 632-777, wherein a staple is —(CH₂)m-N(R′)—(CH₂)n-CH═CH—(CH₂)n′-, wherein each of m, n and n′ is independently 1, 2, 3, 4, 5, 6, 7, 8, 9, or 10, and each —CH₂— is independently optionally substituted.
779. The agent of any one of Embodiments 632-777, wherein a staple is —(CH₂)m-N(R′)—(CH₂)n-CH═CH—(CH₂)n′-, wherein each of m, n and n′ is independently 1, 2, 3, 4, 5, 6, 7, 8, 9, or 10.
780. The agent of any one of Embodiments 632-779, wherein a staple is —(CH₂)m-N(R′)—C(O)—O—(CH₂)n-CH═CH—(CH₂)n′-, wherein each of m, n and n′ is independently 1, 2, 3, 4, 5, 6, 7, 8, 9, or 10, and each —CH₂— is independently optionally substituted.
781. The agent of any one of Embodiments 632-779, wherein a staple is —(CH₂)m-N(R′)—C(O)—O—(CH₂)n-CH═CH—(CH₂)n′-, wherein each of m, n and n′ is independently 1, 2, 3, 4, 5, 6, 7, 8, 9, or 10.
782. The agent of any one of Embodiments 778-781, wherein the —CH═CH— is E.
783. The agent of any one of Embodiments 778-781, wherein the —CH═CH— is Z.
784. The agent of any one of Embodiments 632-783, wherein a staple is —(CH₂)m-N(R′)—(CH₂)n-CH₂—CH₂—(CH₂)n′-, wherein each of m, n and n′ is independently 1, 2, 3, 4, 5, 6, 7, 8, 9, or 10, and each —CH₂— is independently optionally substituted.
785. The agent of any one of Embodiments 632-784, wherein a staple is —(CH₂)m-N(R′)—(CH₂)n-CH₂—CH₂—(CH₂)n′-, wherein each of m, n and n′ is independently 1, 2, 3, 4, 5, 6, 7, 8, 9, or 10.
786. The agent of any one of Embodiments 632-785, wherein a staple is —(CH₂)m-N(R′)—C(O)—O—(CH₂)n-CH₂—CH₂—(CH₂)n′-, wherein each of m, n and n′ is independently 1, 2, 3, 4, 5, 6, 7, 8, 9, or 10, and each —CH₂— is independently optionally substituted.
787. The agent of any one of Embodiments 632-786, wherein a staple is —(CH₂)m-N(R′)—C(O)—O—(CH₂)n-CH₂—CH₂—(CH₂)n′-, wherein each of m, n and n′ is independently 1, 2, 3, 4, 5, 6, 7, 8, 9, or 10.
788. The agent of any one of Embodiments 778-787, wherein —(CH₂)m- is bonded an amino acid residue closer to a N-terminus to the other amino acid residue bonded to the same staple.
789. The agent of any one of Embodiments 778-787, wherein —(CH₂)m- is bonded an amino acid residue closer to a C-terminus to the other amino acid residue bonded to the same staple.
790. The agent of any one of Embodiments 778-789, wherein m is 1.
791. The agent of any one of Embodiments 778-789, wherein m is 2.
792. The agent of any one of Embodiments 778-791, wherein n is 3.
793. The agent of any one of Embodiments 778-792, wherein n′ is 3.
794. The agent of any one of Embodiments 778-793, wherein R′ is —H.
795. The agent of any one of Embodiments 778-793, wherein R′ is optionally substituted C₁₋₆ aliphatic.
796. The agent of any one of Embodiments 778-793, wherein R′ is methyl.
797. The agent of any one of Embodiments 778-793, wherein R′ is taken together with R^{a3} of the amino acid residue to which L^{s3} is bonded to and their intervening atom(s) to form an optionally substituted 3-10 membered ring having 0-5 heteroatoms in addition to the intervening atom(s).
798. The agent of any one of Embodiments 778-793, wherein R′ is taken together with R^{a3} of the amino acid residue to which L^{s3} is bonded to and their intervening atom(s) to form a 3-10 membered monocyclic ring having 0-5 heteroatoms in addition to the intervening atom(s).
799. The agent of any one of Embodiments 797-798, wherein the formed ring is saturated.
800. The agent of any one of Embodiments 797-799, wherein the formed ring is 4-membered.
801. The agent of any one of Embodiments 797-800, wherein the formed ring is 5-membered.
802. The agent of any one of Embodiments 797-801, wherein the formed ring has no heteroatoms in addition to the intervening atom(s).
803. The agent of any one of Embodiments 632-802, wherein a staple is optionally substituted —*CH₂—N(—CH₂—**CH₂—)—C(O)O—(CH₂)₃—CH═CH—(CH₂)₃—, wherein —*CH₂— and —**CH₂— are bonded to the same amino acid residue.
804. The agent of any one of Embodiments 632-802, wherein a staple is —*CH₂—N(—CH₂—**CH₂—)—C(O)O—(CH₂)₃—CH═CH—(CH₂)₃—, wherein —*CH₂— and —**CH₂— are bonded to the same amino acid residue.
805. The agent of Embodiment 803-804, wherein —CH═CH— is E.
806. The agent of Embodiment 803-804, wherein —CH═CH— is Z.
807. The agent of any one of Embodiments 632-802, wherein a staple is optionally substituted —*CH₂—N(—CH₂—**CH₂—)—C(O)O—(CH₂)₃—CH₂—CH₂—(CH₂)₃—, wherein —*CH₂— and —**CH₂— are bonded to the same amino acid residue.
808. The agent of any one of Embodiments 632-802, wherein a staple is —*CH₂—N(—CH₂—**CH₂—)—C(O)O—(CH₂)₃—CH₂—CH₂—(CH₂)₃—, wherein —*CH₂— and —**CH₂— are bonded to the same amino acid residue.
809. The agent of any one of Embodiments 803-808, wherein —*CH₂— and —**CH₂— are bonded to the same atom.
810. The agent of any one of Embodiments 778-809, wherein optionally substituted —(CH₂)m or —*CH₂— is bonded to an amino acid residue closer to a C-terminus to the other amino acid residue bonded to the same staple.
811. The agent of any one of Embodiments 778-810, wherein optionally substituted —(CH₂)m or —*CH₂— is bonded to an alpha-carbon atom of an amino acid residue.
812. The agent of any one of Embodiments 778-811, wherein optionally substituted —(CH₂)m or —*CH₂— is bonded to X¹¹.
813. The agent of Embodiment 812, wherein the other amino acid residue bonded to the staple is X⁴.
814. The agent of any one of Embodiments 632-813, wherein a staple is optionally substituted —(CH₂)m-CH═CH—(CH₂)n-.
815. The agent of any one of Embodiments 632-813, wherein a staple is —(CH₂)m-CH═CH—(CH₂)n-.
816. The agent of any one of Embodiments 632-813, wherein a staple is optionally substituted —(CH₂)m-CH₂—CH₂—(CH₂)n-.
817. The agent of any one of Embodiments 632-813, wherein a staple is —(CH₂)m-CH₂—CH₂—(CH₂)n-.
818. The agent of any one of Embodiments 814-817, wherein m is 1.
819. The agent of any one of Embodiments 814-817, wherein m is 2.
820. The agent of any one of Embodiments 814-817, wherein m is 3.
821. The agent of any one of Embodiments 814-817, wherein m is 4.
822. The agent of any one of Embodiments 814-817, wherein m is 5.
823. The agent of any one of Embodiments 814-817, wherein m is 6.
824. The agent of any one of Embodiments 814-817, wherein m is 7.
825. The agent of any one of Embodiments 814-817, wherein m is 8.
826. The agent of any one of Embodiments 814-825, wherein n is 1.
827. The agent of any one of Embodiments 814-825, wherein n is 2.
828. The agent of any one of Embodiments 814-825, wherein n is 3.
829. The agent of any one of Embodiments 814-825, wherein n is 4.
830. The agent of any one of Embodiments 814-825, wherein n is 5.
831. The agent of any one of Embodiments 814-825, wherein n is 6.
832. The agent of any one of Embodiments 814-825, wherein n is 7.
833. The agent of any one of Embodiments 814-825, wherein n is 8.
834. The agent of any one of Embodiments 814-833, wherein the staple is boned to X⁴ and X¹¹.
835. The agent of any one of Embodiments 632-834, wherein a staple is —(CH₂)m-N(R′)—(CH₂)n-, wherein each of m and n is independently 1, 2, 3, 4, 5, 6, 7, 8, 9, or 10, and each —CH₂— is independently optionally substituted.
836. The agent of any one of Embodiments 632-835, wherein a staple is —(CH₂)m-N(R′)—(CH₂)n-, wherein each of m and n is independently 1, 2, 3, 4, 5, 6, 7, 8, 9, or 10.
837. The agent of any one of Embodiments 632-836, wherein a staple is —(CH₂)m-N(R′)—C(O)—O—(CH₂)n-, wherein each of m and n is independently 1, 2, 3, 4, 5, 6, 7, 8, 9, or 10, and each —CH₂— is independently optionally substituted.
838. The agent of any one of Embodiments 632-837, wherein a staple is —(CH₂)m-N(R′)—C(O)—O—(CH₂)n-, wherein each of m and n is independently 1, 2, 3, 4, 5, 6, 7, 8, 9, or 10.
839. The agent of any one of Embodiments 835-838, wherein R′ is —H.
840. The agent of any one of Embodiments 835-838, wherein R′ is optionally substituted C₁₋₆ aliphatic.
841. The agent of any one of Embodiments 835-838, wherein R′ is methyl.
842. The agent of any one of Embodiments 632-834, wherein a staple is —(CH₂)m-L^{s2}-(CH₂)n-, wherein each of m and n is independently 1, 2, 3, 4, 5, 6, 7, 8, 9, or 10.
843. The agent of Embodiment 842, wherein L^{s2} is optionally substituted

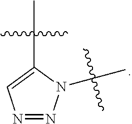 [SMILES: *c1cnnn1*]

844. The agent of Embodiment 842, wherein L^{s2} is

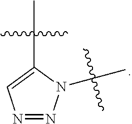 [SMILES: *c1cnnn1*]

845. The agent of Embodiment 842, wherein L^{s2} is optionally substituted

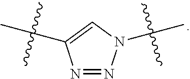 [SMILES: *c1cn(*)nn1]

846. The agent of Embodiment 842, wherein L^{s2} is

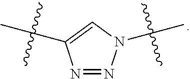 [SMILES: *c1cn(*)nn1]

847. The agent of any one of Embodiments 842-845, wherein the carbon atom is bonded to —(CH₂)m-.
848. The agent of Embodiment 842, wherein L^{s2} is —C(O)—N(R′)—(CH₂)ₙ—N(R′)—C(O)—, where n is 1, 2, 3, 4, 5, 6, 7, 8, 9, or 10, and each —CH₂— is independently optionally substituted.
849. The agent of Embodiment 842, wherein L^{s2} is —C(O)—N(R′)—(CH₂)ₙ—N(R′)—C(O)—, where n is 1, 2, 3, 4, 5, 6, 7, 8, 9, or 10.
850. The agent of Embodiment 842, wherein L^{s2} is —N(R′)—(CH₂)ₙ—N(R′)—, where n is 1, 2, 3, 4, 5, 6, 7, 8, 9, or 10, and each —CH₂— is independently optionally substituted.
851. The agent of Embodiment 842, wherein L^{s2} is —N(R′)—(CH₂)ₙ—N(R′)—, where n is 1, 2, 3, 4, 5, 6, 7, 8, 9, or 10.
852. The agent of Embodiment 842, wherein L^{s2} is —C(O)—N(R′)—(CH₂)ₙ₁—C(R′)₂—(CH₂)ₙ₂—N(R′)—C(O)—, where each of n1 and n2 is independently 1, 2, 3, 4, 5, 6, 7, 8, 9, or 10, and each —CH₂— is independently optionally substituted.
853. The agent of Embodiment 842, wherein L^{s2} is —C(O)—N(R′)—(CH₂)ₙ₁—C(R′)₂—(CH₂)ₙ₂—N(R′)—C(O)—, where each of n1 and n2 is independently 1, 2, 3, 4, 5, 6, 7, 8, 9, or 10.
854. The agent of Embodiment 842, wherein L^{s2} is —N(R′)—(CH₂)ₙ₁—C(R′)₂—(CH₂)ₙ₂—N(R′)—, where each of n1 and n2 is independently 1, 2, 3, 4, 5, 6, 7, 8, 9, or 10, and each —CH₂— is independently optionally substituted.
855. The agent of Embodiment 842, wherein L^{s2} is —N(R′)—(CH₂)ₙ₁—C(R′)₂—(CH₂)ₙ₂—N(R′)—, where each of n1 and n2 is independently 1, 2, 3, 4, 5, 6, 7, 8, 9, or 10.
856. The agent of any one of Embodiments 848-855, wherein each R′ is independently —H or optionally substituted C₁₋₆ aliphatic.
857. The agent of any one of Embodiments 848-855, wherein two R′ are taken together with their intervening atom(s) to form an optionally substituted 3-10 membered ring having 0-5 heteroatoms in addition to the intervening atom(s).
858. The agent of Embodiment 857, wherein one R′ of —C(R′)₂— and one R′ of —N(R′)— or —N(R′)C(O)O— are taken together with their intervening atom(s) to form an optionally substituted 3-10 membered ring having 0-5 heteroatoms in addition to the intervening atom(s).
859. The agent of Embodiment 857, wherein one R′ of —C(R′)₂— and one R′ of —N(R′)— or —N(R′)C(O)O— are taken together with their intervening atom(s) to form a monocyclic 3-10 membered ring having 0-5 heteroatoms in addition to the intervening atom(s).
860. The agent of any one of Embodiments 857-859, wherein the formed ring is saturated.
861. The agent of any one of Embodiments 857-860, wherein the formed ring is 4-membered.
862. The agent of any one of Embodiments 857-860, wherein the formed ring is 5-membered.
863. The agent of any one of Embodiments 857-860, wherein the formed ring is 6-membered.
864. The agent of any one of Embodiments 857-863, wherein the formed ring has no heteroatoms in addition to the intervening atom(s).
865. The agent of any one of Embodiments 859-864, wherein the other R′ of —C(R′)₂— and one R′ of —N(R′)— or —N(R′)C(O)O— are taken together with their intervening atom(s) to form an optionally substituted 3-10 membered ring having 0-5 heteroatoms in addition to the intervening atom(s).
866. The agent of any one of Embodiments 859-865, wherein the other R′ of —C(R′)₂— and one R′ of —N(R′)— or —N(R′)C(O)O— are taken together with their intervening atom(s) to form a monocyclic 3-10 membered ring having 0-5 heteroatoms in addition to the intervening atom(s).
867. The agent of any one of Embodiments 865-866, wherein the formed ring is saturated.
868. The agent of any one of Embodiments 865-867, wherein the formed ring is 4-membered.
869. The agent of any one of Embodiments 865-867, wherein the formed ring is 5-membered.
870. The agent of any one of Embodiments 865-867, wherein the formed ring is 6-membered.
871. The agent of any one of Embodiments 865-870, wherein the formed ring has no heteroatoms in addition to the intervening atom(s).
872. The agent of any one of Embodiments 852-871, wherein n1 is 1.
873. The agent of any one of Embodiments 852-871, wherein n1 is 2.
874. The agent of any one of Embodiments 852-873, wherein n2 is 1.
875. The agent of any one of Embodiments 852-873, wherein n2 is 2.
876. The agent of any one of Embodiments 632-834, wherein a staple is —S—CH₂-Cy-CH₂—S—, wherein each —CH₂— is independently optionally substituted.
877. The agent of Embodiment 876, wherein L^{s2} is —S—CH₂-Cy-CH₂—S—
878. The agent of any one of Embodiments 632-834, wherein a staple is —S—CH₂-Cy-Cy-CH₂—S—, wherein each —CH₂— is independently optionally substituted.
879. The agent of Embodiment 878, wherein L^{s2} is —S—CH₂-Cy-Cy-CH₂—S—.
880. The agent of any one of Embodiments 876-879, wherein -Cy- is optionally substituted phenylene.
881. The agent of Embodiment 880, wherein -Cy- is 1,2-phenylene.
882. The agent of Embodiment 880, wherein -Cy- is 1,3-phenylene.
883. The agent of Embodiment 880, wherein -Cy- is 1,4-phenylene.
884. The agent of any one of Embodiments 632-834, wherein a staple is —S-Cy-Cy-S—.
885. The agent of any one of Embodiments 632-834, wherein a staple is —S-Cy-S—.
886. The agent of any one of Embodiments 884-885, wherein each -Cy- is optionally substituted phenylene.
887. The agent of any one of Embodiments 884-885, wherein each -Cy- is 1,4-tetrafluorophenylene.
888. The agent of any one of Embodiments 632-834, wherein a staple is —C(O)-Cy-C(O)—.
889. The agent of Embodiment 888, wherein -Cy- is optionally substituted monocyclic or bicyclic 5-12 membered ring, wherein each —C(O)— is independently bonded to a nitrogen atom.
890. The agent of any one of Embodiments 632-834, wherein a staple is —N(R′)—C(O)-L″-C(O)—N(R′)—.
891. The agent of Embodiment 890, wherein L^{s1} is -Cy-.
892. The agent of Embodiment 890, wherein L^{s1} is optionally substituted phenylene.
893. The agent of Embodiment 890, wherein L^{s1} is optionally substituted 1,3-phenylene.
894. The agent of Embodiment 890, wherein L^{s1} is optionally substituted bivalent C₁₋₆ aliphatic.
895. The agent of Embodiment 890, wherein L^{s1} is optionally substituted —(CH₂)n-, wherein n is 1, 2, 3, 4, 5, 6, 7, 8, 9, or 10.
896. The agent of Embodiment 890, wherein L^{s1} is —CH₂—CH₂—.
897. The agent of Embodiment 890, wherein L^{s1} is —C(CH₃)₂—.
898. The agent of Embodiment 890, wherein L^{s1} is -Cy-Cy-.
899. The agent of Embodiment 898, wherein each -Cy- is independently optionally substituted phenylene.
900. The agent of Embodiment 898, wherein each -Cy- is independently 1,2-phenylene.
901. The agent of Embodiment 898, wherein each -Cy- is independently 1,3-phenylene.
902. The agent of any one of Embodiments 890-901, wherein each R′ of —N(R′)— is independently —H or optionally substituted C₁₋₆ aliphatic.
903. The agent of any one of Embodiments 890-901, wherein each R′ of —N(R′)— is independently —H.
904. The agent of any one of Embodiments 632-834, wherein a staple is —(CH₂)m-O—CH₂-L^{s2}-(CH₂)n-, wherein each —CH₂— is independently optionally substituted.
905. The agent of Embodiment 889, wherein a staple is —(CH₂)m-O—CH₂-L^{s2}-(CH₂)n-.
906. The agent of any one of Embodiments 889-905, wherein L^{s2} is -Cy-.
907. The agent of Embodiment 906, wherein -Cy- is optionally substituted

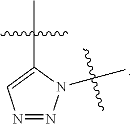 [SMILES: *c1cnnn1*]

908. The agent of Embodiment 906, wherein -Cy- is

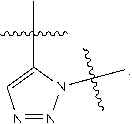 [SMILES: *c1cnnn1*]

909. The agent of Embodiment 906, wherein -Cy- is optionally substituted

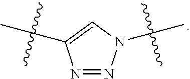 [SMILES: *c1cn(*)nn1]

910. The agent of Embodiment 906, wherein -Cy- is

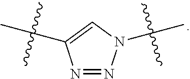 [SMILES: *c1cn(*)nn1]

911. The agent of any one of Embodiments 909-910, wherein the carbon atom is bonded to —(CH₂)n-which is bonded to an amino acid residue.
912. The agent of any one of Embodiments 909-910, wherein the carbon atom is bonded to —CH₂— which is bonded to an —O—.
913. The agent of any one of Embodiments 835-912, wherein —(CH₂)m- is bonded an amino acid residue closer to a N-terminus to the other amino acid residue bonded to the same staple.
914. The agent of any one of Embodiments 835-912, wherein —(CH₂)m- is bonded an amino acid residue closer to a C-terminus to the other amino acid residue bonded to the same staple.
915. The agent of any one of Embodiments 835-914, wherein m is 1.
916. The agent of any one of Embodiments 835-914, wherein m is 2.
917. The agent of any one of Embodiments 835-914, wherein m is 3.
918. The agent of any one of Embodiments 835-914, wherein m is 4.
919. The agent of any one of Embodiments 835-918, wherein n is 1.
920. The agent of any one of Embodiments 835-918, wherein n is 2.
921. The agent of any one of Embodiments 835-918, wherein n is 3.
922. The agent of any one of Embodiments 835-918, wherein n is 4.
923. The agent of any one of Embodiments 632-922, wherein the agent comprises a staple that is optionally substituted —(CH₂)₂C(O)NH(CH₂)₄—.
924. The agent of any one of Embodiments 632-923, wherein the agent comprises a staple that is —(CH₂)₂C(O)NH(CH₂)₄—.
925. The agent of any one of Embodiments 632-924, wherein a staple is optionally substituted

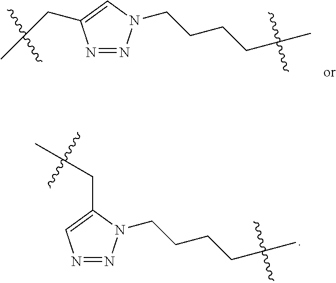 [SMILES: *CCCCn1cc(C*)nn1 ; *CCCCn1nncc1C*]

926. The agent of any one of Embodiments 632-925, wherein a staple is

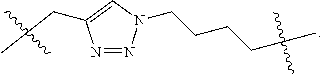 [SMILES: *CCCCn1cc(C*)nn1]

927. The agent of any one of Embodiments 632-925, wherein a staple is

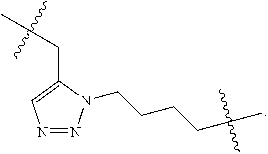 [SMILES: *CCCCn1nncc1C*]

928. The agent of any one of Embodiments 632-927, wherein a staple is optionally substituted

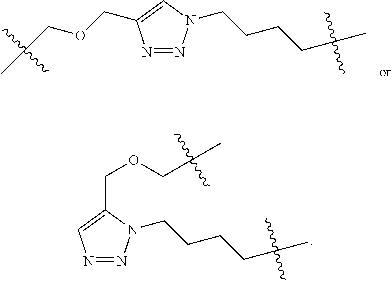 [SMILES: *CCCCn1cc(COC*)nn1 ; *CCCCn1nncc1COC*]

929. The agent of any one of Embodiments 632-928, wherein a staple is

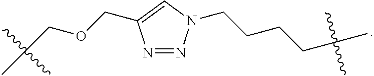 [SMILES: *CCCCn1cc(COC*)nn1]

930. The agent of any one of Embodiments 632-928, wherein a staple is

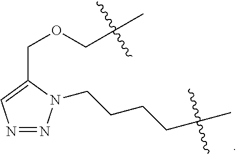 [SMILES: *CCCCn1nncc1COC*]

931. The agent of any one of Embodiments 923-930, wherein optionally substituted —(CH₂)₄— is bonded to an amino acid residue closer to a N-terminus to the other amino acid residue bonded to the same staple.
932. The agent of any one of Embodiments 923-930, wherein optionally substituted —(CH₂)₄— is bonded to an amino acid residue closer to a C-terminus to the other amino acid residue bonded to the same staple.
933. The agent of any one of Embodiments 632-932, wherein a staple is optionally substituted —S—CH₂-(1,3-phenylene)-CH₂—S—.
934. The agent of any one of Embodiments 632-933, wherein a staple is —S—CH₂-(1,3-phenylene)-CH₂—S—.
935. The staple of any one of Embodiments 835-934, wherein the staple is bonded to X³ and X⁷.
936. The staple of any one of Embodiments 835-934, wherein the staple is bonded to X³ and X¹⁰.
937. The staple of any one of Embodiments 835-934, wherein the staple is bonded to X⁷ and X¹⁰.
938. The staple of any one of Embodiments 835-934, wherein the staple is bonded to X⁷ and X¹⁴.
939. The staple of any one of Embodiments 835-934, wherein the staple is bonded to X¹⁰ and X¹⁴.
940. The agent of any one of the preceding Embodiments, wherein a staple has a length of 5-10 chain atoms.
941. The agent of Embodiment 940, wherein the length is 5 chain atoms.
942. The agent of Embodiment 940, wherein the length is 6 chain atoms.
943. The agent of Embodiment 940, wherein the length is 7 chain atoms.
944. The agent of any one of Embodiments 940-943, wherein the staple is a (i, i+2) staple.
945. The agent of any one of Embodiments 940-943, wherein the staple is a (i, i+3) staple.
946. The agent of any one of the preceding Embodiments, wherein a staple has a length of 7-12 chain atoms.
947. The agent of Embodiment 946, wherein the length is 7 chain atoms.
948. The agent of Embodiment 946, wherein the length is 8 chain atoms.
949. The agent of Embodiment 946, wherein the length is 9 chain atoms.
950. The agent of any one of Embodiments 946-949, wherein the staple is a (i, i+3) staple.
951. The agent of any one of the preceding Embodiments, wherein a staple has a length of 10-25 chain atoms.
952. The agent of Embodiment 951, wherein the length is 12 chain atoms.
953. The agent of Embodiment 951, wherein the length is 13 chain atoms.
954. The agent of Embodiment 951, wherein the length is 14 chain atoms.
955. The agent of any one of Embodiments 951-954, wherein the staple is a (i, i+7) staple.
956. The agent of any one of Embodiments 630-955, wherein the three staples are within 10-20 consecutive amino acid residues.
957. The agent of any one of Embodiments 630-955, wherein the three staples are within 14 consecutive amino acid residues.
958. The agent of any one of Embodiments 630-955, wherein the three staples are within 11 consecutive amino acid residues.
959. The agent of any one of Embodiments 630-958, wherein the first staple connects two residues at positions i and i+2.
960. The agent of any one of Embodiments 630-958, wherein the first staple connects two residues at positions i and i+3.
961. The agent of any one of Embodiments 630-960, wherein the second staple connects two residues at positions i+3 and i+10.
962. The agent of any one of Embodiments 630-961, wherein the third staple connects two residues at positions i+9 and i+13.
963. The agent of any one of Embodiments 630-962, wherein the third staple connects two residues at positions i+6 and i+9.
964. The agent of any one of Embodiments 630-963, wherein the third staple connects two residues at positions i+6 and i+13.
965. The agent of any one of Embodiments 630-964, wherein the peptide comprises a fourth staple.
966. The agent of any one of Embodiments 630-965, wherein the fourth staple connects two residues at positions i+2 and i+6.
967. The agent of any one of Embodiments 630-966, wherein the first staple has the structure of -L^{s1}-L^{s2}-L^{s3}-.
968. The agent of any one of Embodiments 630-967, wherein the second staple has the structure of -L^{s1}-L^{s2}-L^{s3}-.
969. The agent of any one of Embodiments 630-968, wherein the third staple has the structure of -L^{s1}-L^{s2}-L^{s3}-.
970. The agent of any one of Embodiments 630-969, wherein the fourth staple has the structure of -L^{s1}-L^{s2}-L^{s3}-.
971. The agent of any one of the preceding Embodiments, comprising a first staple comprising a (E)-double bond.
972. The agent of any one of the preceding Embodiments, comprising a first staple comprising a (Z)-double bond.
973. The agent of any one of the preceding Embodiments, comprising a second staple comprising a (E)-double bond.
974. The agent of any one of the preceding Embodiments, comprising a second staple comprising a (Z)-double bond.
975. The agent of any one of the preceding Embodiments, comprising a third staple comprising a (E)-double bond.
976. The agent of any one of the preceding Embodiments, comprising a third staple comprising a (Z)-double bond.
977. The agent of any one of the preceding Embodiments, wherein the staple between X¹ and X⁴ has the structure of -L^{s1}-L^{s2}-L^{s3}- wherein each of L^{s1}, L^{s2} and L^{s3} is independently a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
978. The agent of any one of the preceding Embodiments, wherein the staple between X⁴ and Xu has the structure of -L^{s}-L^{s2}-L^{s3}-, wherein each of L^{s1}, L^{s2} and L^{s3} is independently a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
979. The agent of any one of the preceding Embodiments, wherein the staple between X¹⁰ and X¹⁴ has the structure of -L^{s}L-L^{s2}-L^{s3}-, wherein each of L^{s1}, L^{s2} and L^{s3} is independently a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
980. The agent of any one of the preceding Embodiments, wherein the staple between X⁷ and X¹⁰ has the structure of -L^{s}-L^{s2}-L^{s3}-, wherein each of L^{s1}, L^{s2} and L^{s3} is independently a covalent bond, or an optionally substituted bivalent linear or branched, saturated or partially unsaturated Co hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
981. The agent of any one of the preceding Embodiments, wherein the staple between X⁷ and X⁴ has the structure of -L^{s1}-L^{s2}-L^{s3}- wherein each of L^{s1}, L^{s2} and L^{s3} is independently a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
982. The agent of any one of the preceding Embodiments, wherein the staple between X³ and X⁷ has the structure of -L^{s}-L^{s2}-L^{s3}-, wherein each of L^{s1}, L^{s2} and L^{s3} is independently a covalent bond, or an optionally substituted bivalent linear or branched, saturated or partially unsaturated C₁₋₁₀ hydrocarbon chain, wherein one or more methylene units are optionally and independently replaced with —C(R′)₂—, -Cy-, —O—, —S—, —S—S—, —S-Cy-S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
983. The agent of any one of the preceding Embodiments, wherein L^{s1} a covalent bond, or an optionally substituted bivalent linear or branched, saturated or partially unsaturated C₁₋₁₀ hydrocarbon chain, wherein one or more methylene units are optionally and independently replaced with —O—.
984. The agent of any one of the preceding Embodiments, wherein L^{s1} is an optionally substituted bivalent linear or branched, saturated or partially unsaturated C₁₋₁₀ hydrocarbon chain.
985. The agent of any one of the preceding Embodiments, wherein L^{s2} is -Cy-.
986. The agent of any one of the preceding Embodiments, wherein L^{s2} is an optionally substituted triazolylene ring.
987. The agent of any one of Embodiments 1-984, wherein L^{s2} is or comprises —C(O)—.
988. The agent of any one of Embodiments 1-984, wherein L^{s2} is or comprises —C(O)N(R′)—.
989. The agent of any one of Embodiments 1-984 and 988, wherein L^{s2} is —C(O)NH—.
990. The agent of any one of Embodiments 1-984 and 988, wherein L^{s2} is —C(O)N(R′)—, wherein R′ is C₁₋₆ aliphatic.
991. The agent of any one of Embodiments 1-984, wherein L^{s2} is —S-Cy-S—.
992. The agent of any one of Embodiments 1-984 and 991, wherein L^{s2} is —S-Cy-S—, wherein -Cy- is an optionally substituted monocyclic or bicyclic arylene ring.
993. The agent of any one of Embodiments 1-984 and 991-992, wherein L^{s2} is —S-Cy-S—, wherein -Cy- is an optionally substituted phenylene ring.
994. The agent of any one of Embodiments 1-984 and 991-992, wherein L^{s2} is —S-Cy-S—, wherein -Cy- is an optionally substituted biphenylene ring.
995. The agent of any one of the preceding Embodiments, wherein L^{s3} a covalent bond, or an optionally substituted bivalent linear or branched, saturated or partially unsaturated C₁₋₁₀ hydrocarbon chain, wherein one or more methylene units are optionally and independently replaced with —O—.
996. The agent of any one of the preceding Embodiments, wherein L^{s3} is or comprises an optionally substituted bivalent linear or branched, saturated or partially unsaturated C₁₋₁₀ hydrocarbon chain.
997. The agent of any one of the preceding Embodiments, wherein L^{s1} is bonded to an atom of the peptide backbone.
998. The agent of any one of the preceding Embodiments, wherein L^{s1} is bonded to an a-carbon of an amino acid residue.
999. The agent of any one of the preceding Embodiments, wherein L^{s1} is bonded to a ring atom of a ring, wherein the ring comprises one or more ring atoms that are atoms of the peptide backbone.
1000. The agent of any one of the preceding Embodiments, wherein L^{s1} is bonded to a ring atom of a ring, wherein the ring comprises an a-carbon of an amino acid residue.
1001. The agent of any one of the preceding Embodiments, wherein a methylene unit of L^{s1} is replaced with —C(R′)₂—, wherein one R′ of —C(R′)₂— and R′ attached to the backbone are taken together with their intervening atom(s) to form an optionally substituted 3-10 membered ring having 0-5 heteroatoms.
1002. The agent of any one of the preceding Embodiments, wherein methylene unit of L^{s1} is replaced with —N(R′)—, wherein one R′ of the —N(R′)— and R′ attached to the backbone are taken together with their intervening atom(s) to form an optionally substituted 3-10 membered ring having 0-5 heteroatoms.
1003. The agent of any one of the preceding Embodiments, wherein R′ attached to the backbone is R^{a2} of an amino acid.
1004. The agent of any one of the preceding Embodiments, wherein R′ is attached to an atom of the same residue to which L^{s1} is bonded.
1005. The agent of any one of the preceding Embodiments, wherein L^{s3} is bonded to an atom of the peptide backbone.
1006. The agent of any one of the preceding Embodiments, wherein L^{s3} is bonded to an a-carbon of an amino acid residue.
1007. The agent of any one of the preceding Embodiments, wherein L^{s3} is bonded to a ring atom of a ring, wherein the ring comprises one or more ring atoms that are atoms of the peptide backbone.
1008. The agent of any one of the preceding Embodiments, wherein L^{s3} is bonded to a ring atom of a ring, wherein the ring comprises an a-carbon of an amino acid residue.
1009. The agent of any one of the preceding Embodiments, wherein a methylene unit of L^{s3} is replaced with —C(R′)₂—, wherein one R′ of —C(R′)₂— and R′ attached to the backbone are taken together with their intervening atom(s) to form an optionally substituted 3-10 membered ring having 0-5 heteroatoms.
1010. The agent of any one of the preceding Embodiments, wherein methylene unit of L^{s3} is replaced with —N(R′)—, wherein one R′ of the —N(R′)— and R′ attached to the backbone are taken together with their intervening atom(s) to form an optionally substituted 3-10 membered ring having 0-5 heteroatoms. (e.g., R^{a2} or the other group attached to alpha-carbon is R′).
1011. The agent of any one of the preceding Embodiments, wherein R′ attached to the backbone is R^{a2} of an amino acid.
1012. The agent of any one of the preceding Embodiments, wherein R′ is attached to an atom of the same residue to which L^{s3} is bonded.
1013. The agent of any one of the preceding Embodiments, wherein R′ is attached to the same atom as L^{s3}.
1014. The agent of any one of the preceding Embodiments, wherein p0 is 1.
1015. The agent of any one of the preceding Embodiments, wherein X⁰ is a residue of an amino acid that comprises an olefin.
1016. The agent of any one of the preceding Embodiments, wherein X⁰ is a residue of an amino acid that comprises —CH═CH₂.
1017. The agent of any one of the preceding Embodiments, wherein X⁰ is a residue of an amino acid that comprises —CH═CH₂ and forms a staple with another amino acid residue through olefin metathesis.
1018. The agent of any one of the preceding Embodiments, wherein X⁰ is a residue of an amino acid having the structure of formula A-I, A-II or A-III, wherein R^{a2} or R^{a3} comprises an olefin
1019. The agent of any one of the preceding Embodiments, wherein X⁰ is a residue of an amino acid having the structure of formula A-I, A-II or A-III, wherein R^{a2} or R^{a3} is -L^{a}-CH═CH₂.
1020. The agent of any one of the preceding Embodiments, wherein X⁰ is S5 or S6.
1021. The agent of any one of the preceding Embodiments, wherein X⁰ is stapled with X⁴.
1022. The agent of any one of Embodiments 1-1014, wherein X⁰ is selected from Gly, Sar, and NMebAla.
1023. The agent of any one of Embodiments 1-1013, wherein p0 is 0.
1024. The agent of any one of the preceding Embodiments, wherein X² is selected from Asp, Asn, Hse, Glu, Aad, Ser, aThr, Thr, MeAsn, SbMeAsp, RbMeAsp, aMeDAsp, and OAsp.
1025. The agent of any one of the preceding Embodiments, wherein X² is selected from Asp, Asn, Hse, Glu, Aad, Ser, and aThr.
1026. The agent of any one of the preceding Embodiments, wherein X² comprises a side chain comprising an acidic group.
1027. The agent of any one of the preceding Embodiments, wherein X² comprises a side chain comprising —COOH or a salt form thereof.
1028. The agent of any one of the preceding Embodiments, wherein X² is Asp.
1029. The agent of any one of Embodiments 1-1025, wherein X² comprises a side chain comprising a polar group.
1030. The agent of any one of Embodiments 1-1025 and 1029, wherein X² comprises a side chain comprising an amidyl group.
1031. The agent of any one of the preceding Embodiments, wherein X² is —N(R^{a1})-L^{a1}-C(R^{a1})(R^{a3})-L^{a2}-C(O)—.
1032. The agent of Embodiment 1031, wherein R^{a1} is —H.
1033. The agent of any one of Embodiments 1031-1032, wherein R^{a3} is —H.
1034. The agent of any one of Embodiments 1031-1032, wherein R^{a3} is optionally substituted C₁₋₆ aliphatic.
1035. The agent of any one of Embodiments 1031-1034, wherein L^{a1} is a covalent bond
1036. The agent of any one of Embodiments 1031-1035, wherein L^{a2} is a covalent bond.
1037. The agent of any one of Embodiments 1031-1036, wherein R^{a2} is or comprises an acidic or polar group.
1038. The agent of any one of Embodiments 1031-1037, wherein R^{a2} is -L″-COOH.
1039. The agent of any one of Embodiments 1031-1037, wherein R^{a2} is -L″-Cy-COOH.
1040. The agent of Embodiment 1039, wherein -Cy- is optionally substituted phenylene.
1041. The agent of any one of Embodiments 1031-1037, wherein R^{a2} is -L″-C(O)N(R′)₂.
1042. The agent of any one of Embodiments 1038-1041, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1043. The agent of any one of Embodiments 1038-1041, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1044. The agent of any one of Embodiments 1038-1041, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1045. The agent of any one of Embodiments 1038-1042, wherein L^{s1} is optionally substituted —(CH₂)n- wherein n is 1, 2, 3, 4, 5, or 6.
1046. The agent of any one of Embodiments 1038-1045, wherein L^{s1} is —(CH₂)n- wherein n is 1, 2, 3, 4, 5, or 6.
1047. The agent of any one of Embodiments 1-1025 and 1029-1030, wherein X² is Asn.
1048. The agent of any one of Embodiments 1-1025 and 1029, wherein X² comprises a side chain comprising —OH.
1049. The agent of any one of Embodiments 1-1025, 1029, and 1048, wherein X² is Hse.
1050. The agent of any one of Embodiments 1-1023, wherein X² is Asp, Ala, Asn, Glu, Npg, Ser, Hse, Val, S5, S6, AcLys, TfeGA, aThr, Aad, Pro, Thr, Phe, Leu, PL3, Gln, isoGlu, MeAsn, isoDAsp, RbGlu, SbGlu, AspSH, Ile, SbMeAsp, RbMeAsp, aMeDAsp, OAsp, 3COOHF, NAsp, 3Thi, NGlu, isoDGlu, BztA, Tle, Aib, MePro, Chg, Cha, or DipA.
1051. The agent of any one of the preceding Embodiments, wherein X² interacts with Gly307 of beta-catenin or an amino acid residue corresponding thereto.
1052. The agent of any one of the preceding Embodiments, wherein X² interacts with Lys312 of beta-catenin or an amino acid residue corresponding thereto.
1053. The agent of any one of the preceding Embodiments, wherein X³ is selected from Npg, Leu, Cha, Val, nLeu, Ile, Phe, CypA, CyLeu, Chg, Pff, DiethA, Ala, Tyr, Trp, Ser, Aib, Phg, DipA, OctG, Cba, MorphNva, and F2PipNva.
1054. The agent of any one of the preceding Embodiments, wherein X³ comprises one or two hydrophobic side chains.
1055. The agent of any one of the preceding Embodiments, wherein X³ is —N(R^{a1})-L^{a1}-C(R^{a2})(R^{a3})-L^{a2}-C(O)—.
1056. The agent of Embodiment 1055, wherein X³ is —N(R^{a1})—C(R^{a2})(R^{a3})—C(O)—.
1057. The agent of Embodiment 1055, wherein X³ is —NH—C(R^{a2})(R^{a3})—C(O)—.
1058. The agent of any one of Embodiments 1055-1057, wherein R^{a2} and R^{a3} are independently hydrogen or optionally substituted C₁₋₁₀ aliphatic.
1059. The agent of any one of Embodiments 1055-1057, wherein one of R^{a2} and R^{a3} is hydrogen and the other is C₁₋₁₀ aliphatic.
1060. The agent of any one of Embodiments 1055-1057, wherein R^{a2} and R^{a3} are taken together with the carbon atom to which they are attached to form an optionally substituted 3-8 membered ring having 1-3 heteroatoms.
1061. The agent of any one of Embodiments 1055-1057, wherein R^{a2} and R^{a3} are taken together with the carbon atom to which they are attached to form 3-8 membered cycloalkyl.
1062. The agent of any one of the preceding Embodiments, wherein the side chain of X³ is C₁₋₁₀ alkyl optionally substituted with one or more substituents independently selected from -Cy- and —OR, wherein -Cy- is an optionally substituted bivalent, 3-10 membered, monocyclic, bicyclic or polycyclic ring having 0-5 heteroatoms;
   R is independently C₁₋₄ alkyl; or
   two C₁₋₁₀ alkyl groups are taken together with their intervening atom(s) to form an optionally substituted 3-10 membered ring having 0-5 heteroatoms in addition to the intervening atom(s).
1063. The agent of any one of the preceding Embodiments, wherein the side chain of X³ is C₁₋₁₀ alkyl.
1064. The agent of any one of the preceding Embodiments, wherein X³ is not stapled.
1065. The agent of any one of Embodiments 1-1052, wherein X³ is Npg, Ile, Asp, Cha, DipA, Chg, Leu, B5, Cba, S5, Ala, Glu, AllylGly, nLeu, Ser, B6, Asn, B4, GlnR, Val, [Phc][Allyl]Dap, Hse, [Bn][Allyl]Dap, 1MeK, R5, Phe, CypA, CyLeu, Pff, DiethA, Tyr, Trp, Aib, Phg, OctG, MorphNva, F2PipNva, [Piv][Allyl]Dap, [CyCO][Allyl]Dap, Lys, or S3.
1066. The agent of any one of Embodiments 1-1052, wherein X³ is Npg.
1067. The agent of any one of Embodiments 1-1052, wherein X³ is Ile.
1068. The agent of any one of Embodiments 1-1052, wherein X³ is Cha.
1069. The agent of any one of Embodiments 1-1052, wherein X³ is DipA.
1070. The agent of any one of Embodiments 1-1052, wherein X³ is Chg.
1071. The agent of any one of Embodiments 1-1052, wherein X³ is Leu.
1072. The agent of any one of Embodiments 1-1052, wherein X³ is B5.
1073. The agent of any one of Embodiments 1-1052, wherein X³ is Asp.
1074. The agent of any one of Embodiments 1-1052, wherein X³ is Cba.
1075. The agent of any one of Embodiments 1-1052, wherein X³ is S5.
1076. The agent of any one of Embodiments 1-1052, wherein X³ is Ala.
1077. The agent of any one of the preceding Embodiments, wherein X³ interacts with Tyr306 of beta-catenin or an amino acid residue corresponding thereto.
1078. The agent of any one of the preceding Embodiments, wherein X⁵ is selected from Asp, Glu, Asn, Hse, aThr, Aad, Ser, Thr, MeAsn, SbMeAsp, and RbMeAsp.
1079. The agent of any one of the preceding Embodiments, wherein X⁵ is —N(R^{a1})-L^{a1}-C(R^{a2})(R^{a3})-L^{a2}-C(O)—.
1080. The agent of Embodiment 1079, wherein R^{a1} is —H.
1081. The agent of any one of Embodiments 1079-1080, wherein R^{a3} is —H.
1082. The agent of any one of Embodiments 1079-1080, wherein R^{a3} is optionally substituted C₁₋₆ aliphatic.
1083. The agent of any one of Embodiments 1079-1082, wherein L^{a1} is a covalent bond.
1084. The agent of any one of Embodiments 1079-1083, wherein L^{a2} is a covalent bond.
1085. The agent of any one of Embodiments 1079-1084, wherein R^{a2} is or comprises an acidic or polar group.
1086. The agent of any one of Embodiments 1079-1085, wherein R^{a2} is -L″-COOH.
1087. The agent of any one of Embodiments 1079-1085, wherein R^{a2} is -L″-Cy-COOH.
1088. The agent of Embodiment 1087, wherein -Cy- is optionally substituted phenylene.
1089. The agent of any one of Embodiments 1079-1085, wherein R^{a2} is -L″-C(O)N(R′)₂.
1090. The agent of any one of Embodiments 1086-1089, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1091. The agent of any one of Embodiments 1086-1089, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1092. The agent of any one of Embodiments 1086-1089, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1093. The agent of any one of Embodiments 1086-1090, wherein L^{s1} is optionally substituted —(CH₂)n- wherein n is 1, 2, 3, 4, 5, or 6.
1094. The agent of any one of Embodiments 1086-1090, wherein L^{s1} is —(CH₂)n- wherein n is 1, 2, 3, 4, 5, or 6. 1095. The agent of any one of the preceding Embodiments, wherein X⁵ comprises a side chain comprising an acidic group.
1096. The agent of any one of the preceding Embodiments, wherein X⁵ comprises a side chain comprising —COOH or a salt form thereof.
1097. The agent of any one of the preceding Embodiments, wherein X⁵ is Asp.
1098. The agent of any one of Embodiments 1-1078, wherein X⁵ comprises a side chain comprising a polar group.
1099. The agent of any one of Embodiments 1-1078 and 1098, wherein X⁵ comprises a side chain comprising —OH.
1100. The agent of any one of Embodiments 1-1078 and 1098, wherein X⁵ comprises a side chain comprising an amidyl group.
1101. The agent of any one of Embodiments 1-1077, wherein X⁵ is selected from 3COOHF, TfeGA, Asp, Gln, [CH2CMe2CO2H]TriAzDap, Thr, Glu, 2OH3COOHF, 40H3COOHF, 4COOHF, 2COOHF, His, Tyr, 5F3Me2COOHF, 4F3Me2COOHF, 5F3Me3COOHF, 4F3Me3COOHF, 3F2COOHF, Val, Ser, Trp, Asn, Ala, Arg, dGlu, aThr, hTyr, 3cbmf, Leu, Phe, Lys, and Ile.
1102. The agent of any one of Embodiments 1-1077, wherein X⁵ is Asp, B5, 3COOHF, Glu, Asn, Npg, Hse, aThr, Aad, Ser, Thr, MeAsn, AspSH, SbMeAsp or RbMeAsp.
1103. The agent of any one of Embodiments 1-1077, wherein X⁵ is B5.
1104. The agent of any one of Embodiments 1-1077, wherein X⁵ is 3COOHF.
1105. The agent of any one of Embodiments 1-1077, wherein X⁵ is Glu.
1106. The agent of any one of the preceding Embodiments, wherein X⁵ interacts with Trp383 of beta-catenin or an amino acid residue corresponding thereto.
1107. The agent of any one of the preceding Embodiments, wherein X⁵ interacts with Arg386 of beta-catenin or an amino acid residue corresponding thereto.
1108. The agent of any one of the preceding Embodiments, wherein X⁵ interacts with Asn387 of beta-catenin or an amino acid residue corresponding thereto.
1109. The agent of any one of the preceding Embodiments, wherein X⁶ is —N(R^{a1})-L^{a1}-C(R^{a2})(R^{a3})-L^{a2}-C(O)—.
1110. The agent of Embodiment 1109, wherein R^{a1} is —H.
1111. The agent of any one of Embodiments 1109-1110, wherein R^{a3} is —H
1112. The agent of any one of Embodiments 1109-1110, wherein R^{a3} is optionally substituted C₁₋₆ aliphatic.
1113. The agent of any one of Embodiments 1109-1112, wherein L^{a1} is a covalent bond.
1114. The agent of any one of Embodiments 1109-1113, wherein L^{a2} is a covalent bond.
1115. The agent of any one of Embodiments 1109-1114, wherein R^{a2} is or comprises an acidic or polar group.
1116. The agent of any one of Embodiments 1109-1115, wherein R^{a2} is -L″-COOH.
1117. The agent of any one of Embodiments 1109-1115, wherein R^{a2} is -L″-Cy-COOH.
1118. The agent of Embodiment 1117, wherein -Cy- is optionally substituted phenylene.
1119. The agent of any one of Embodiments 1109-1115, wherein R^{a2} is -L″-C(O)N(R′)₂.
1120. The agent of any one of Embodiments 1116-1119, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1121. The agent of any one of Embodiments 1116-1119, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1122. The agent of any one of Embodiments 1116-1119, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1123. The agent of any one of Embodiments 1116-1120, wherein L^{s1} is optionally substituted —(CH₂)n- wherein n is 1, 2, 3, 4, 5, or 6.
1124. The agent of any one of Embodiments 1116-1123, wherein L^{s1} is —(CH₂)n- wherein n is 1, 2, 3, 4, 5, or 6.
1125. The agent of any one of Embodiments 1116-1122, wherein a methylene unit is replaced with —N(R′)—.
1126. The agent of Embodiment 1125, wherein R′ is —H.
1127. The agent of Embodiment 1125, wherein R′ is optionally substituted C₁₋₆ alkyl.
1128. The agent of any one of the preceding Embodiments, wherein X⁶ comprises a side chain comprising an acidic or a polar group.
1129. The agent of any one of the preceding Embodiments, wherein X⁶ comprises a side chain comprising an acidic group.
1130. The agent of any one of the preceding Embodiments, wherein X⁶ comprises a side chain comprising —COOH or a salt form thereof.
1131. The agent of any one of the preceding Embodiments, wherein X⁶ is 3COOHF.
1132. The agent of any one of Embodiments 1-1130, wherein X⁶ is TfeGA.
1133. The agent of any one of Embodiments 1-1130, wherein X⁶ is Asp.
1134. The agent of any one of Embodiments 1-1130, wherein X⁶ is [CH2CMe2CO2H]TriAzDap.
1135. The agent of any one of Embodiments 1-1109, wherein X⁶ comprises a side chain comprising a polar group.
1136. The agent of any one of Embodiments 1-1109 and 1135, wherein X⁶ comprises a side chain comprising —OH.
1137. The agent of any one of Embodiments 1-1109 and 1135, wherein X⁶ comprises a side chain comprising an amidyl group.
1138. The agent of any one of Embodiments 1-1109, 1135, and 1137, wherein X⁶ is Gln.
1139. The agent of any one of the preceding Embodiments, wherein X⁶ interacts with Tyr306 of beta-catenin or an amino acid residue corresponding thereto.
1140. The agent of any one of the preceding Embodiments, wherein X⁶ interacts with Lys345 of beta-catenin or an amino acid residue corresponding thereto.
1141. The agent of any one of the preceding Embodiments, wherein X⁷ is a hydrophobic amino acid residue.
1142. The agent of any one of the preceding Embodiments, wherein X⁷ is —N(R^{a1})-L^{a1}-C(R^{a2})(R^{a3})-L^{a2}-C(O)—.
1143. The agent of any one of the preceding Embodiments, wherein X⁷ is —N(R^{a1})—C(R^{a2})(R^{a3})—C(O)—.
1144. The agent of any one of the preceding Embodiments, wherein X⁷ is —NH—C(R^{a2})(R^{a3})—C(O)—.
1145. The agent of any one of Embodiments 1142-1144, wherein R^{a2} and R^{a3} are independently hydrogen or optionally substituted C₁₋₁₀ aliphatic.
1146. The agent of any one of Embodiments 1142-1144, wherein one of R^{a2} and R^{a3} is hydrogen and the other is C₁₋₁₀ aliphatic.
1147. The agent of any one of Embodiments 1142-1144, wherein R^{a2} and R^{a3} are taken together with the carbon atom to which they are attached to form an optionally substituted 3-8 membered ring having 1-3 heteroatoms.
1148. The agent of any one of Embodiments 1142-1144, wherein R^{a2} and R^{a3} are taken together with the carbon atom to which they are attached to form 3-8 membered cycloalkyl.
1149. The agent of any one of the preceding Embodiments, wherein X⁷ is selected from Aib, Ala, MorphGln, Gln, Ser, iPrLys, nLeu, Cha, Hse, Npg, Val, CyLeu, Thr, Phe, Acp, Asn, DaMeS, aMeDF, Leu, Cpg, Cbg, Me2Gln, Met20, AcLys, His, aMeL, DaMeL, aMeV, aMeS, and aMeF.
1150. The agent of any one of the preceding Embodiments, wherein X⁷ is selected from Aib, Ala, MorphGln, Gln, Ser, iPrLys, nLeu, Cha, Hse, Npg, Val, and CyLeu.
1151. The agent of any one of the preceding Embodiments, wherein X⁷ is selected from Aib, Ala, MorphGln, Gln, Ser, iPrLys, nLeu, Cha, and Hse.
1152. The agent of any one of the preceding Embodiments, wherein X⁷ is Aib.
1153. The agent of any one of Embodiments 1-1140, wherein X⁷ is Ala.
1154. The agent of any one of Embodiments 1-1140, wherein X⁷ is CyLeu.
1155. The agent of any one of Embodiments 1-1140, wherein X⁷ is Phe.
1156. The agent of any one of Embodiments 1-1140, wherein X⁷ is nLeu.
1157. The agent of any one of Embodiments 1-1140, wherein X⁷ is Val.
1158. The agent of any one of the preceding Embodiments, wherein X¹¹ is a hydrophobic amino acid residue.
1159. The agent of any one of the preceding Embodiments, wherein X¹¹ is —N(R^{a1})-L^{a1}-C(R^{a2})(R^{a3})-L^{a2}-C(O)—.
1160. The agent of any one of the preceding Embodiments, wherein X⁸ is —N(R^{a1})—C(R^{a2})(R^{a3})—C(O)—.
1161. The agent of any one of the preceding Embodiments, wherein X⁸ is —NH—C(R^{a2})(R^{a3})—C(O)—.
1162. The agent of any one of Embodiments 1159-1161, wherein R^{a2} and R^{a3} are independently hydrogen or optionally substituted C₁₋₁₀ aliphatic.
1163. The agent of any one of Embodiments 1159-1161, wherein one of R^{a2} and R^{a3} is hydrogen and the other is C₁₋₁₀ aliphatic.
1164. The agent of any one of Embodiments 1159-1161, wherein R^{a2} and R^{a3} are taken together with the carbon atom to which they are attached to form an optionally substituted 3-8 membered ring having 1-3 heteroatoms.
1165. The agent of any one of Embodiments 1159-1161, wherein R^{a2} and R^{a3} are taken together with the carbon atom to which they are attached to form 3-8 membered cycloalkyl.
1166. The agent of any one of the preceding Embodiments, wherein X¹¹ is selected from Ala, Aib, Cpg, Val, Leu, Gln, Lys, Asp, Glu, Aad, nLeu, Cba, Ser, Thr, aThr, MorphGln, Phe, hPhe, hTyr, and AcLys.
1167. The agent of any one of Embodiments 1-1157, wherein X¹¹ is Ala, Aib, Phe, Asp, 3COOHF, aThr, Gly, Ser, nLeu, Thr, Cpg, Val, Leu, Gln, Lys, Glu, Aad, Cba, MorphGln, hPhe, hTyr, or AcLys.
1168. The agent of any one of the preceding Embodiments, wherein X¹¹ is Ala.
1169. The agent of any one of Embodiments 1-1157, wherein X¹¹ is Aib.
1170. The agent of any one of Embodiments 1-1157, wherein X¹¹ is Phe.
1171. The agent of any one of Embodiments 1-1157, wherein X¹¹ is Asp.
1172. The agent of any one of Embodiments 1-1157, wherein X¹¹ is 3COOHF.
1173. The agent of any one of the preceding Embodiments, wherein X⁸ interacts with Trp383 of beta-catenin or an amino acid residue corresponding thereto.
1174. The agent of any one of the preceding Embodiments, wherein X⁹ is selected from Phe, 3COOHF, 2NapA, nLeu, Tyr, 3Thi, 4FF, 4ClF, 4BrF, 3FF, 3ClF, 3BrF, 2FF, 30MeF, 4CNF, 3CNF, 4MeF, 3MeF, Aic, RbiPrF, SbiPrF, RbiPrDF, RbMeXylA, RbMeXylDA, Cba, CypA, BztA, 1NapA, Trp, Leu, Ile, Ser, 2Thi, Chg, Hse, 4TriA, 3F3MeF, Thr, His, Val, Asn, Gln, 2Cpg, SbMeXylA, and SbMeXylDA.
1175. The agent of any one of the preceding Embodiments, wherein X⁹ comprises a side chain which is or comprises an optionally substituted aromatic group.
1176. The agent of any one of the preceding Embodiments, wherein X⁹ is —N(R^{a1})-L^{a1}-C(R^{a2})(R^{a3}-L^{a2}-C(O)—.
1177. The agent of Embodiment 1176, wherein R^{a1} is —H.
1178. The agent of any one of Embodiments 1176-1177, wherein R^{a3} is —H.
1179. The agent of any one of Embodiments 1176-1177, wherein R^{a3} is optionally substituted C₁₋₆ aliphatic.
1180. The agent of any one of Embodiments 1176-1179, wherein L^{a1} is a covalent bond.
1181. The agent of any one of Embodiments 1176-1180, wherein R^{a2} is -L^{a}-R, wherein R is or comprises an aromatic group.
1182. The agent of Embodiment 1181, wherein R is optionally substituted 6-10 membered aryl.
1183. The agent of Embodiment 1181, wherein R is optionally substituted phenyl.
1184. The agent of Embodiment 1181, wherein R is phenyl.
1185. The agent of Embodiment 1181, wherein R is optionally substituted naphthyl.
1186. The agent of Embodiment 1181, wherein R is naphthyl.
1187. The agent of Embodiment 1181, wherein R is optionally substituted 5-membered heteroaryl having 1-4 heteroatoms.
1188. The agent of Embodiment 1181, wherein R is optionally substituted 6-membered heteroaryl having 1-4 heteroatoms.
1189. The agent of Embodiment 1181, wherein R is optionally substituted 9-membered bicyclic heteroaryl having 1-4 heteroatoms.
1190. The agent of Embodiment 1181, wherein R is optionally substituted 10-membered bicyclic heteroaryl having 1-4 heteroatoms.
1191. The agent of any one of Embodiments 1187-1190, wherein a heteroatom is nitrogen.
1192. The agent of any one of Embodiments 1187-1191, wherein a heteroatom is oxygen.
1193. The agent of any one of Embodiments 1187-1192, wherein a heteroatom is sulfur.
1194. The agent of any one of Embodiments 1187-1190, wherein the heteroaryl has only one heteroatom.
1195. The agent of Embodiment 1194, wherein the heteroatom is nitrogen.
1196. The agent of Embodiment 1194, wherein the heteroatom is oxygen.
1197. The agent of Embodiment 1194, wherein the heteroatom is sulfur.
1198. The agent of any one of Embodiments 1181-1197, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1199. The agent of any one of Embodiments 1181-1197, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1200. The agent of any one of Embodiments 1181-1197, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1201. The agent of Embodiment 1198, wherein L^{a} is optionally substituted —(CH₂)n- wherein n is 1, 2, 3, 4, 5, or 6.
1202. The agent of Embodiment 1198, wherein L^{a} is —(CH₂)n- wherein n is 1, 2, 3, 4, 5, or 6.
1203. The agent of Embodiment 1198, wherein L^{a} is —CH₂—.
1204. The agent of any one of the preceding Embodiments, wherein X⁹ comprises a side chain which is or comprises an optionally substituted aromatic group, wherein each optional substituent of the aromatic group is independently selected from halogen, —OR, —R, —C(O)OR, and —CN, wherein each R is independently —H, C₁₋₄ alkyl, or haloalkyl.
1205. The agent of any one of the preceding Embodiments, wherein X⁹ comprises a side chain which is or comprises an optionally substituted aromatic group, wherein each optional substituent of the aromatic group is independently selected from halogen, —OR, —R, —C(O)OH, and —CN, wherein each R is independently C₁₋₄ alkyl or haloalkyl.
1206. The agent of any one of the preceding Embodiments, wherein X⁹ comprises a side chain which is or comprises an optionally substituted aromatic group, wherein each optional substituent of the aromatic group is independently selected from halogen, —OR, —R, —C(O)OH, and —CN, wherein each R is independently C₁₋₂ alkyl or haloalkyl.
1207. The agent of any one of the preceding Embodiments, wherein X⁹ comprises a side chain which is or comprises an optionally substituted aromatic group, wherein each optional substituent of the aromatic group is independently selected from halogen, —OR, —R, —C(O)OH, and —CN, wherein each R is independently methyl optionally substituted with one or more halogen.
1208. The agent of any one of the preceding Embodiments, wherein X⁹ comprises a side chain which is or comprises an optionally substituted aromatic group, wherein each optional substituent of the aromatic group is independently selected from halogen, —OR, —R, —C(O)OH, and —CN, wherein each R is independently methyl optionally substituted with one or more F.
1209. The agent of any one of the preceding Embodiments, wherein X⁹ comprises a side chain which is or comprises an optionally substituted aromatic group, wherein each optional substituent of the aromatic group is independently selected from —F, —Cl, —Br, —OCH₃, —CH₃, —CF₃, —C(O)OH, and —CN.
1210. The agent of any one of the preceding Embodiments, wherein X⁹ comprises a side chain which is or comprises an unsubstituted aromatic group.
1211. The agent of any one of Embodiments 1-1173, wherein X⁹ is AA9, Phe, Ala, Lys, 3COOHF, Aib, 2NapA, nLeu, 2Thi, Tyr, 3Thi, 4FF, 4ClF, 4BrF, 3FF, 3ClF, 3BrF, 2FF, 30MeF, 4CNF, 3CNF, 4MeF, 3MeF, Aic, RbiPrF, SbiPrF, RbiPrDF, RbMeXylA, RbMeXylDA, Cba, CypA, BztA, 1NapA, Trp, Leu, Ile, Ser, Chg, Hse, 4TriA, 3F3MeF, Thr, His, Val, Asn, Gln, 2Cpg, SbMeXylA, or SbMeXylDA.
1212. The agent of any one of Embodiments 1-1173, wherein X⁹ is Phe.
1213. The agent of any one of Embodiments 1-1173, wherein X⁹ is Ala.
1214. The agent of any one of Embodiments 1-1173, wherein X⁹ is Lys.
1215. The agent of any one of Embodiments 1-1173, wherein X⁹ is 3COOHF.
1216. The agent of any one of Embodiments 1-1173, wherein X⁹ is Aib.
1217. The agent of any one of the preceding Embodiments, wherein X⁹ interacts with Lys345 of beta-catenin or an amino acid residue corresponding thereto.
1218. The agent of any one of the preceding Embodiments, wherein X⁹ interacts with Trp383 of beta-catenin or an amino acid residue corresponding thereto.
1219. The agent of any one of the preceding Embodiments, wherein X¹⁰ is not stapled.
1220. The agent of any one of the preceding Embodiments, wherein X¹⁰ is —N(R^{a1})-L^{a1}-C(R^{a2})(R^{a3})-L^{a2}-C(O)—.
1221. The agent of Embodiment 1220, wherein R^{a1} is —H.
1222. The agent of any one of Embodiments 1220-1221, wherein R^{a3} is —H.
1223. The agent of any one of Embodiments 1220-1221, wherein R^{a3} is optionally substituted C₁₋₆ aliphatic.
1224. The agent of any one of Embodiments 1220-1223, wherein L^{a1} is a covalent bond.
1225. The agent of any one of Embodiments 1220-1224, wherein L^{a2} is a covalent bond.
1226. The agent of any one of Embodiments 1220-1225, wherein R^{a2} is -L″-R.
1227. The agent of any one of Embodiments 1220-1225, wherein R^{a2} is -L″-Cy-R.
1228. The agent of any one of Embodiments 1226-1227, wherein R is hydrogen or optionally substituted C₁₋₁₀ aliphatic.
1229. The agent of any one of Embodiments 1226-1227, wherein R is optionally substituted C₁₋₁₀ aliphatic.
1230. The agent of any one of Embodiments 1226-1227, wherein R is C₁₋₁₀ aliphatic.
1231. The agent of any one of Embodiments 1226-1227, wherein R is C₁₋₁₀ alkyl.
1232. The agent of any one of Embodiments 1226-1227, wherein R is optionally substituted phenyl.
1233. The agent of any one of Embodiments 1220-1225, R^{a2} is -L″-C(O)N(R′)₂.
1234. The agent of any one of Embodiments 1220-1225, R^{a2} is -L″-OH.
1235. The agent of any one of Embodiments 1220-1234, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1236. The agent of any one of Embodiments 1220-1234, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1237. The agent of any one of Embodiments 1220-1234, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1238. The agent of any one of Embodiments 1220-1234, wherein L^{s1} is optionally substituted —(CH₂)n- wherein n is 1, 2, 3, 4, 5, or 6.
1239. The agent of any one of Embodiments 1220-1234, wherein L^{s1} is —(CH₂)n- wherein n is 1, 2, 3, 4, 5, or 6.
1240. The agent of any one of Embodiments 1-1218, wherein X¹⁰ is Lys, Phe, TriAzLys, GlnR, Leu, PyrS2, Aib, Ala, sAla, AsnR, hGlnR, dOm, PyrS1, dLys, dDab, [mPyr]Cys, PyrS3, iPrLys, [mXyl]Cys, TriAzOm, 1MeK, [C3]Cys, [IsoE]Cys, DGlnR, Orn, [mPyr]hCys, [Red] Cys, [C3]hCys, 4PipA, sCH2S, [8FBB]Cys, [pXyl]Cys, [pXyl]hCys, [33Oxe]Cys, [Red]hCys, [IsoE]hCys, [13Ac]hCys, [m5Meb]Cys, [m5Meb]hCys, GlnS3APyr, AsnMeEDA, AsnR3APyr, [m5Pyr]Cys, [m50Meb]Cys, [4FB]Cys, [oXyl]Cys, NMeOm, [2-6-naph]Cys, [3-3-biph]Cys, [mXyl]hCys, [3-3-biPh]hCys, [2-6-naph]hCys, [33Oxe]hCys, [13Ac]Cys, GlnR3APyr, AsnS3APyr, [IsoE]hCysOx, or [m5Pyr]hCys.
1241. The agent of any one of Embodiments 1-1218, wherein X¹⁰ is Lys.
1242. The agent of any one of Embodiments 1-1218, wherein X¹⁰ is Phe.
1243. The agent of any one of Embodiments 1-1218, wherein X¹⁰ is TriAzLys.
1244. The agent of any one of Embodiments 1-1218, wherein X¹⁰ is GlnR.
1245. The agent of any one of Embodiments 1-1218, wherein X¹⁰ is Leu.
1246. The agent of any one of Embodiments 1-1218, wherein X¹⁰ is PyrS2.
1247. The agent of any one of Embodiments 1-1218, wherein X¹⁰ is Aib.
1248. The agent of any one of Embodiments 1-1218, wherein X¹⁰ is Ala.
1249. The agent of any one of Embodiments 1-1218, wherein X¹⁰ is Leu.
1250. The agent of any one of the preceding Embodiments, wherein X¹² is selected from 3Thi, 2F3MeF, Phe, nLeu, 2COOHF, CypA, 2ClF, Ala, Abu, Leu, hLeu, Npg, Cpa, Nva, Cba, ChA, 2FurA, 20MeF, 2MeF, 2BrF, 2CNF, 2N02F, 2PyrA, 3PyrA, 4PyrA, His, 1NapA, Val, Ile, Chg, DiethA, hnLeu, OctG, 2Thi, and 2cbmF.
1251. The agent of any one of the preceding Embodiments, wherein X¹² comprises a side chain which is or comprises an optionally substituted aromatic group.
1252. The agent of any one of the preceding Embodiments, wherein X¹² is —N(R^{a1})-L^{a1}-C(R^{a2})(R^{a3})-L^{a2}-C(O)—.
1253. The agent of Embodiment 1252, wherein R^{a1} is —H.
1254. The agent of any one of Embodiments 1252-1253, wherein R^{a3} is —H.
1255. The agent of any one of Embodiments 1252-1253, wherein R^{a3} is optionally substituted C₁₋₆ aliphatic.
1256. The agent of any one of Embodiments 1252-1255, wherein L^{a1} is a covalent bond.
1257. The agent of any one of Embodiments 1252-1256, wherein R^{a2} is -L^{a}-R, wherein R is or comprises an aromatic group.
1258. The agent of Embodiment 1257, wherein R is optionally substituted 6-10 membered aryl
1259. The agent of Embodiment 1257, wherein R is optionally substituted phenyl
1260. The agent of Embodiment 1257, wherein R is phenyl
1261. The agent of Embodiment 1257, wherein R is optionally substituted naphthyl
1262. The agent of Embodiment 1257, wherein R is naphthyl
1263. The agent of Embodiment 1257, wherein R is optionally substituted 5-membered heteroaryl having 1-4 heteroatoms
1264. The agent of Embodiment 1257, wherein R is optionally substituted 6-membered heteroaryl having 1-4 heteroatoms
1265. The agent of Embodiment 1257, wherein R is optionally substituted 9-membered bicyclic heteroaryl having 1-4 heteroatoms
1266. The agent of Embodiment 1257, wherein R is optionally substituted 10-membered bicyclic heteroaryl having 1-4 heteroatoms
1267. The agent of any one of Embodiments 1263-1266, wherein a heteroatom is nitrogen
1268. The agent of any one of Embodiments 1263-1267, wherein a heteroatom is oxygen
1269. The agent of any one of Embodiments 1263-1268, wherein a heteroatom is sulfur
1270. The agent of any one of Embodiments 1263-1266, wherein the heteroaryl has only one heteroatom
1271. The agent of Embodiment 1270, wherein the heteroatom is nitrogen.
1272. The agent of Embodiment 1270, wherein the heteroatom is oxygen.
1273. The agent of Embodiment 1270, wherein the heteroatom is sulfur.
1274. The agent of any one of Embodiments 1257-1273, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1275. The agent of any one of Embodiments 1257-1273, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1276. The agent of any one of Embodiments 1257-1273, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1277. The agent of Embodiment 1274, wherein L^{a} is optionally substituted —(CH₂)n- wherein n is 1, 2, 3, 4, 5, or 6.
1278. The agent of Embodiment 1274, wherein L^{a} is —(CH₂)n- wherein n is 1, 2, 3, 4, 5, or 6.
1279. The agent of Embodiment 1274, wherein L^{a} is —CH₂—.
1280. The agent of any one of the preceding Embodiments, wherein X¹² comprises a side chain which is or comprises an optionally substituted aromatic group, wherein each optional substituent of the aromatic group is independently selected from halogen, —OR, —R, —C(O)OR, —C(O)N(R)₂, —CN, and —NO₂, wherein each R is independently —H, C₁₋₄ alkyl, or haloalkyl.
1281. The agent of any one of the preceding Embodiments, wherein X¹² comprises a side chain which is or comprises an optionally substituted aromatic group, wherein each optional substituent of the aromatic group is independently selected from halogen, —OR, —R, —C(O)OH, —C(O)NH₂, —CN, and —NO₂, wherein each R is independently C₁₋₄ alkyl or haloalkyl.
1282. The agent of any one of the preceding Embodiments, wherein X¹² comprises a side chain which is or comprises an optionally substituted aromatic group, wherein each optional substituent of the aromatic group is independently selected from halogen, —OR, —R, —C(O)OH, —C(O)NH₂, —CN, and —NO₂, wherein each R is independently C₁₋₂ alkyl or haloalkyl.
1283. The agent of any one of the preceding Embodiments, wherein X¹² comprises a side chain which is or comprises an optionally substituted aromatic group, wherein each optional substituent of the aromatic group is independently selected from halogen, —OR, —R, —C(O)OH, —C(O)NH₂, —CN, and —NO₂, wherein each R is independently methyl optionally substituted with one or more halogen.
1284. The agent of any one of the preceding Embodiments, wherein X¹² comprises a side chain which is or comprises an optionally substituted aromatic group, wherein each optional substituent of the aromatic group is independently selected from halogen, —OR, —R, —C(O)OH, —C(O)NH₂, —CN, and —NO₂, wherein each R is independently methyl optionally substituted with one or more —F.
1285. The agent of any one of the preceding Embodiments, wherein X¹² comprises a side chain which is or comprises an optionally substituted aromatic group, wherein each optional substituent of the aromatic group is independently selected from —Br, —OCH₃, —CH₃, —CF₃, —C(O)OH, —C(O)NH₂, —CN, or —NO₂.
1286. The agent of any one of the preceding Embodiments, wherein X¹² comprises a side chain which is or comprises an optionally substituted aromatic group optionally substituted at 2′-position.
1287. The agent of any one of the preceding Embodiments, wherein X¹² comprises a side chain which is or comprises an unsubstituted aromatic group.
1288. The agent of any one of Embodiments 1251-1287, wherein the aromatic group is a 5-membered heteroaryl group.
1289. The agent of any one of the preceding Embodiments, wherein X¹² is 3Thi.
1290. The agent of any one of Embodiments 1251-1287, wherein the aromatic group is a phenyl group.
1291. The agent of any one of Embodiment 1290, wherein X¹² is 2F3MeF.
1292. The agent of any one of Embodiment 1290, wherein X¹² is Phe.
1293. The agent of any one of Embodiment 1290, wherein X¹² is Phe wherein the phenyl is 2′-substituted.
1294. The agent of any one of Embodiment 1290, wherein X¹² is 2F3MeF, 2COOHF, 2ClF, 20MeF, 2MeF, 2BrF, 2CNF, 2N02F, or 2cbmF.
1295. The agent of any one of Embodiments 1-1249, wherein X¹² is 3Thi, Phe, 2F3MeF, PyrS2, 2ClF, hnLeu, BztA, 2Thi, 2MeF, 2FF, 34ClF, Lys, nLeu, 2COOHF, 2PhF, hCbA, hCypA, hCha, CypA, hPhe, DipA, HepG, Dap7Abu, hhLeu, hhSer, HexG, [2IAPAc]2NH2F, Ala, Abu, Leu, hLeu, Npg, Cpa, PyrS1, [Bnc]2NH2F, [Phc]2NH2F, [BiPh]2NH2F, [3PyAc]2NH2F, Nva, Cba, ChA, 2FurA, 20MeF, 2BrF, 2CNF, 2N02F, 2PyrA, 3PyrA, 4PyrA, His, 1NapA, Val, Ile, Chg, DiethA, OctG, 2cbmF, c6Phe, [MePipAc]2NH2F, or [2PyCypCO]2NH2F.
1296. The agent of any one of Embodiments 1-1249, wherein X¹² is 3Thi.
1297. The agent of any one of Embodiments 1-1249, wherein X¹² is Phe.
1298. The agent of any one of Embodiments 1-1249, wherein X¹² is 2F3MeF.
1299. The agent of any one of Embodiments 1-1249, wherein X¹² is PyrS2.
1300. The agent of any one of Embodiments 1-1249, wherein X¹² is 2ClF.
1301. The agent of any one of Embodiments 1-1249, wherein X¹² is hnLeu.
1302. The agent of any one of Embodiments 1-1249, wherein X¹² is BztA.
1303. The agent of any one of Embodiments 1-1249, wherein X¹² is 2Thi.
1304. The agent of any one of Embodiments 1-1249, wherein X¹² is 2MeF.
1305. The agent of any one of Embodiments 1-1249, wherein X¹² is 2FF.
1306. The agent of any one of Embodiments 1-1249, wherein X¹² is 34ClF.
1307. The agent of any one of the preceding Embodiments, wherein X¹² interacts with Trp383 of beta-catenin or an amino acid residue corresponding thereto.
1308. The agent of any one of the preceding Embodiments, wherein X¹² interacts with Asn415 of beta-catenin or an amino acid residue corresponding thereto.
1309. The agent any one of the preceding Embodiments, wherein the side chain of X¹³ comprises an optionally substituted aromatic group.
1310. The agent of any one of the preceding Embodiments, wherein X¹³ is —N(R^{a1})-L^{a1}-C(R^{a2})(R^{a3})-L^{a2}-C(O)—.
1311. The agent of Embodiment 1310, wherein R^{a1} is —H.
1312. The agent of any one of Embodiments 1310-1311, wherein R^{a3} is —H.
1313. The agent of any one of Embodiments 1310-1311, wherein R^{a3} is optionally substituted C₁₋₆ aliphatic.
1314. The agent of any one of Embodiments 1310-1313, wherein L^{a1} is a covalent bond
1315. The agent of any one of Embodiments 1310-1314, wherein R^{a2} is -L^{a}-R, wherein R is or comprises an aromatic group.
1316. The agent of Embodiment 1315, wherein R is optionally substituted 6-10 membered aryl.
1317. The agent of Embodiment 1315, wherein R is optionally substituted phenyl.
1318. The agent of Embodiment 1315, wherein R is phenyl.
1319. The agent of Embodiment 1315, wherein R is optionally substituted naphthyl.
1320. The agent of Embodiment 1315, wherein R is naphthyl.
1321. The agent of Embodiment 1315, wherein R is optionally substituted 5-membered heteroaryl having 1-4 heteroatoms.
1322. The agent of Embodiment 1315, wherein R is optionally substituted 6-membered heteroaryl having 1-4 heteroatoms.
1323. The agent of Embodiment 1315, wherein R is optionally substituted 9-membered bicyclic heteroaryl having 1-4 heteroatoms.
1324. The agent of Embodiment 1315, wherein R is optionally substituted 10-membered bicyclic heteroaryl having 1-4 heteroatoms.
1325. The agent of any one of Embodiments 1321-1324, wherein a heteroatom is nitrogen.
1326. The agent of any one of Embodiments 1321-1324, wherein a heteroatom is oxygen.
1327. The agent of any one of Embodiments 1321-1324, wherein a heteroatom is sulfur.
1328. The agent of any one of Embodiments 1321-1324, wherein the heteroaryl has only one heteroatom.
1329. The agent of Embodiment 1328, wherein the heteroatom is nitrogen.
1330. The agent of Embodiment 1328, wherein the heteroatom is oxygen.
1331. The agent of Embodiment 1328, wherein the heteroatom is sulfur.
1332. The agent of any one of Embodiments 1315-1331, wherein L^{a} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1333. The agent of any one of Embodiments 1315-1331, wherein L^{a} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1334. The agent of any one of Embodiments 1315-1331, wherein L^{a} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1335. The agent of Embodiment 1332, wherein L^{a} is optionally substituted —(CH₂)n- wherein n is 1, 2, 3, 4, 5, or 6.
1336. The agent of Embodiment 1332, wherein L^{a} is —(CH₂)n- wherein n is 1, 2, 3, 4, 5, or 6.
1337. The agent of Embodiment 1332, wherein L^{a} is —CH₂—.
1338. The agent any one of the preceding Embodiments, wherein the side chain of X¹³ comprises an optionally substituted 8-10 membered bicyclic aromatic group.
1339. The agent any one of the preceding Embodiments, wherein the side chain of X¹³ comprises an optionally substituted 9-membered bicyclic heteroaryl group having 1-3 heteroatoms.
1340. The agent of any one of the preceding Embodiments, wherein X¹³ is BtzA.
1341. The agent of any one of Embodiments 1-1338, wherein X¹³ is 2NapA.
1342. The agent of any one of Embodiments 1309, wherein the aromatic group is a phenyl group.
1343. The agent of any one of Embodiment 1342, wherein X¹³ is 34ClF.
1344. The agent of any one of Embodiments 1-1308, wherein X¹³ is selected from BztA, 34ClF, 2NapA, 3BrF, and 34MeF.
1345. The agent of any one of Embodiments 1-1308, wherein X¹³ is 3Thi.
1346. The agent of any one of Embodiments 1-1308, wherein X¹³ is Phe.
1347. The agent of any one of Embodiments 1-1308, wherein X¹³ is GlnR.
1348. The agent of any one of Embodiments 1-1308, wherein X¹³ is 34MeF.
1349. The agent of any one of Embodiments 1-1308, wherein X¹³ is 2NapA.
1350. The agent of any one of Embodiments 1-1308, wherein X¹³ is Lys.
1351. The agent of any one of the preceding Embodiments, wherein X¹³ interacts with Gln379 of beta-catenin or an amino acid residue corresponding thereto.
1352. The agent of any one of the preceding Embodiments, wherein X¹³ interacts with Leu382 of beta-catenin or an amino acid residue corresponding thereto.
1353. The agent of any one of the preceding Embodiments, wherein X¹³ interacts with Val416 of beta-catenin or an amino acid residue corresponding thereto.
1354. The agent of any one of the preceding Embodiments, wherein X¹³ interacts with Asn415 of beta-catenin or an amino acid residue corresponding thereto.
1355. The agent of any one of the preceding Embodiments, wherein X¹³ interacts with Trp383 of beta-catenin or an amino acid residue corresponding thereto.
1356. The agent of any one of the preceding Embodiments, wherein X¹⁴ is not stapled.
1357. The agent of any one of the preceding Embodiments, wherein X¹⁴ is —N(R^{a1})-L^{a1}-C(R^{a2})(R^{a3})-L^{a2}-C(O)—.
1358. The agent of Embodiment 1357, wherein R^{a1} is —H.
1359. The agent of any one of Embodiments 1357-1358, wherein R^{a3} is —H.
1360. The agent of any one of Embodiments 1357-1358, wherein R^{a3} is optionally substituted C₁₋₆ aliphatic.
1361. The agent of any one of Embodiments 1357-1360, wherein L^{a1} is a covalent bond.
1362. The agent of any one of Embodiments 1357-1361, wherein L^{a2} is a covalent bond.
1363. The agent of any one of Embodiments 1357-1362, wherein R^{a2} is -L″-R.
1364. The agent of any one of Embodiments 1357-1362, wherein R^{a2} is -L″-Cy-R.
1365. The agent of any one of Embodiments 1357-1362, wherein R^{a2} is -L″-C(O)OR.
1366. The agent of any one of Embodiments 1357-1362, wherein R^{a2} is -L″-C(O)N(R′)₂.
1367. The agent of any one of Embodiments 1357-1362, wherein R^{a2} is -L″-C(O)N(R)₂.
1368. The agent of any one of Embodiments 1359-1367, wherein R is hydrogen or optionally substituted C₁₋₁₀ aliphatic.
1369. The agent of any one of Embodiments 1359-1367, wherein R is hydrogen.
1370. The agent of any one of Embodiments 1359-1367, wherein R is optionally substituted C₁₋₁₀ aliphatic.
1371. The agent of any one of Embodiments 1359-1367, wherein R is C₁₋₁₀ aliphatic.
1372. The agent of any one of Embodiments 1359-1367, wherein R is C₁₋₁₀ alkyl.
1373. The agent of any one of Embodiments 1357-1362, wherein R^{a2} is -L″-OH.
1374. The agent of any one of Embodiments 1357-1373, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1375. The agent of any one of Embodiments 1357-1373, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1376. The agent of any one of Embodiments 1357-1373, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1377. The agent of any one of Embodiments 1357-1373, wherein L^{s1} is optionally substituted —(CH₂)n- wherein n is 1, 2, 3, 4, 5, or 6.
1378. The agent of any one of Embodiments 1357-1373, wherein L^{s1} is —(CH₂)n- wherein n is 1, 2, 3, 4, 5, or 6.
1379. The agent of any one of Embodiments 1-1355, wherein X¹⁴ is GlnR.
1380. The agent of any one of Embodiments 1-1355, wherein X¹⁴ is BztA.
1381. The agent of any one of Embodiments 1-1355, wherein X¹⁴ is sAla.
1382. The agent of any one of Embodiments 1-1355, wherein X¹⁴ is 34ClF.
1383. The agent of any one of Embodiments 1-1355, wherein X¹⁴ is Cys.
1384. The agent of any one of Embodiments 1-1355, wherein X¹⁴ is Ala.
1385. The agent of any one of Embodiments 1-1355, wherein X¹⁴ is Lys.
1386. The agent of any one of Embodiments 1-1355, wherein X¹⁴ is AsnR.
1387. The agent of any one of Embodiments 1-1355, wherein X¹⁴ is aMeC.
1388. The agent of any one of Embodiments 1-1355, wherein X¹⁴ is PyrS2.
1389. The agent of any one of Embodiments 1-1355, wherein X¹⁴ is hGlnR.
1390. The agent of any one of Embodiments 1-1355, wherein X¹⁴ is 3Thi.
1391. The agent of any one of Embodiments 1-1355, wherein X¹⁴ is Lys.
1392. The agent of any one of Embodiments 1-1355, wherein X¹⁴ is Gln.
1393. The agent of any one of the preceding Embodiments, wherein X¹⁴ comprises a C-terminal group.
1394. The agent of any one of the preceding Embodiments, wherein X⁵ is —N(R^{a1})-L^{a1}-C(R^{a2})(R^{a3})-L^{a2}-C(O)—.
1395. The agent of Embodiment 1394, wherein R^{a1} is —H.
1396. The agent of any one of Embodiments 1394-1395, wherein R^{a3} is —H.
1397. The agent of any one of Embodiments 1394-1395, wherein R^{a3} is optionally substituted C₁₋₆ aliphatic.
1398. The agent of any one of Embodiments 1394-1397, wherein L^{a1} is a covalent bond.
1399. The agent of any one of Embodiments 1394-1398, wherein L^{a2} is a covalent bond.
1400. The agent of any one of Embodiments 1394-1399, wherein R^{a2} is -L″-R.
1401. The agent of any one of Embodiments 1394-1399, wherein R^{a2} is -L″-Cy-R.
1402. The agent of any one of Embodiments 1394-1399, wherein R^{a2} is -L″-C(O)OR.
1403. The agent of any one of Embodiments 1394-1399, wherein R^{a2} is -L″-C(O)N(R′)₂.
1404. The agent of any one of Embodiments 1394-1399, wherein R^{a2} is -L″-C(O)N(R)₂.
1405. The agent of any one of Embodiments 1400-1404, wherein R is hydrogen or optionally substituted C₁₋₁₀ aliphatic.
1406. The agent of any one of Embodiments 1400-1404, wherein R is hydrogen.
1407. The agent of any one of Embodiments 1400-1404, wherein R is optionally substituted C₁₋₁₀ aliphatic.
1408. The agent of any one of Embodiments 1400-1404, wherein R is C₁₋₁₀ aliphatic.
1409. The agent of any one of Embodiments 1400-1404, wherein R is C₁₋₁₀ alkyl.
1410. The agent of any one of Embodiments 1394-1399, wherein R^{a2} is -L″-OH.
1411. The agent of any one of Embodiments 1394-1410, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1412. The agent of any one of Embodiments 1394-1410, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1413. The agent of any one of Embodiments 1394-1410, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1414. The agent of any one of Embodiments 1394-1410, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1415. The agent of any one of Embodiments 1394-1410, wherein L^{s1} is —(CH₂)n- wherein n is 1, 2, 3, 4, 5, or 6.
1416. The agent of any one of the preceding Embodiments, wherein p15 is 1.
1417. The agent of any one of the preceding Embodiments, wherein X⁵ is selected from Ala, Leu, Val, Aib, MorphNva, Thr, dAla, dLeu, [BiotinPEG8]Lys, Glu, and AzLys.
1418. The agent of any one of the preceding Embodiments, wherein X⁵ comprises a hydrophobic side chain.
1419. The agent of any one of the preceding Embodiments, wherein the side chain of X⁵ is C₁₋₁₀ alkyl.
1420. The agent of any one of the preceding Embodiments, wherein X⁵ is Ala.
1421. The agent of any one of the preceding Embodiments, wherein X¹¹ is optionally substituted or labeled Lys.
1422. The agent of any one of Embodiments 1-1393, wherein X⁵ is Ala, GlnR, Leu, Val, Ser, Thr, 3Thi, BztA, Aib, MorphNva, dAla, dLeu, Pro, Phe, [BiotinPEG8]Lys, Throl, Glu, AzLys, Npg, Trp, Tyr, Lys, Prool, Alaol, Gly, dPro, Asn, Gln, Ala_D3, [mPEG4]Lys, [mPEG8]Lys, or [mPEG16]Lys.
1423. The agent of any one of Embodiments 1-1393, wherein X⁵ is Ala.
1424. The agent of any one of Embodiments 1-1393, wherein X⁵ is optionally substituted or labeled Lys.
1425. The agent of any one of Embodiments 1-1393, wherein X⁵ is GlnR.
1426. The agent of any one of Embodiments 1-1393, wherein X⁵ is Leu.
1427. The agent of any one of Embodiments 1-1393, wherein X⁵ is Val.
1428. The agent of any one of Embodiments 1-1393, wherein X⁵ is Ser.
1429. The agent of any one of Embodiments 1-1393, wherein X⁵ is Thr.
1430. The agent of any one of Embodiments 1-1393, wherein X⁵ is 3Thi.
1431. The agent of any one of Embodiments 1-1393, wherein X⁵ is BztA.
1432. The agent of any one of the preceding Embodiments, wherein X⁵ comprises a C-terminal group.
1433. The agent of any one of Embodiments 1-1393, wherein p15 is 0.
1434. The agent of any one of the preceding Embodiments, wherein p16 is 1.
1435. The agent of any one of the preceding Embodiments, wherein X¹⁶ is —N(R^{a1})-L^{a1}-C(R^{a2})(R^{a3})-L^{a2}-C(O)—.
1436. The agent of Embodiment 1435, wherein R^{a1} is —H.
1437. The agent of any one of Embodiments 1435-1436, wherein R^{a3} is —H.
1438. The agent of any one of Embodiments 1435-1436, wherein R^{a3} is optionally substituted C₁₋₆ aliphatic.
1439. The agent of any one of Embodiments 1435-1438, wherein L^{a1} is a covalent bond.
1440. The agent of any one of Embodiments 1435-1439, wherein L^{a2} is a covalent bond.
1441. The agent of any one of Embodiments 1435-1440, wherein R^{a2} is -L″-R.
1442. The agent of any one of Embodiments 1435-1440, wherein R^{a2} is -L″-Cy-R.
1443. The agent of any one of Embodiments 1435-1440, wherein R^{a2} is -L″-C(O)OR.
1444. The agent of any one of Embodiments 1435-1440, wherein R^{a2} is -L″-C(O)N(R′)₂.
1445. The agent of any one of Embodiments 1435-1440, wherein R^{a2} is -L″-C(O)N(R)₂.
1446. The agent of any one of Embodiments 1441-1445, wherein R is hydrogen or optionally substituted C₁₋₁₀ aliphatic.
1447. The agent of any one of Embodiments 1441-1445, wherein R is hydrogen.
1448. The agent of any one of Embodiments 1441-1445, wherein R is optionally substituted C₁₋₁₀ aliphatic.
1449. The agent of any one of Embodiments 1441-1445, wherein R is C₁₋₁₀ aliphatic.
1450. The agent of any one of Embodiments 1441-1445, wherein R is C₁₋₁₀ alkyl.
1451. The agent of any one of Embodiments 1435-1440, wherein R^{a2} is -L″-OH.
1452. The agent of any one of Embodiments 1435-1451, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1453. The agent of any one of Embodiments 1435-1451, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1454. The agent of any one of Embodiments 1435-1451, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1455. The agent of any one of Embodiments 1435-1451, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1456. The agent of any one of Embodiments 1435-1451, wherein L^{s1} is —(CH₂)n- wherein n is 1, 2, 3, 4, 5, or 6.
1457. The agent of any one of Embodiments 1-1434, wherein X¹⁶ is selected from Ser, Ala, Glu, Aib, Asp, Thr, and aThr.
1458. The agent of any one of Embodiments 1-1434, wherein X¹⁶ is Ala, Ser, Glu, GlnR, BztA, Thr, Aib, Asp, Lys, aThr, Val, or Arg.
1459. The agent of any one of any one of the preceding Embodiments, wherein X¹⁶ comprises a C-terminal group.
1460. The agent of any one of Embodiments 1-1433, wherein p16 is 0.
1461. The agent of any one of the preceding Embodiments, wherein p17 is 1.
1462. The agent of any one of the preceding Embodiments, wherein X⁷ is —N(R^{a1})-L^{a1}-C(R^{a2})(R^{a3})-L^{a2}-C(O)—.
1463. The agent of Embodiment 1462, wherein R^{a1} is —H.
1464. The agent of any one of Embodiments 1462-1463, wherein R^{a3} is —H.
1465. The agent of any one of Embodiments 1462-1463, wherein R^{a3} is optionally substituted C₁₋₆ aliphatic.
1466. The agent of any one of Embodiments 1462-1465, wherein Lai is a covalent bond.
1467. The agent of any one of Embodiments 1462-1466, wherein L^{a2} is a covalent bond.
1468. The agent of any one of Embodiments 1462-1467, wherein R^{a2} is -L″-R.
1469. The agent of any one of Embodiments 1462-1467, wherein R^{a2} is -L″-Cy-R.
1470. The agent of any one of Embodiments 1462-1467, wherein R^{a2} is -L″-C(O)OR.
1471. The agent of any one of Embodiments 1462-1467, wherein R^{a2} is -L″-C(O)N(R′)₂.
1472. The agent of any one of Embodiments 1462-1467, wherein R^{a2} is -L″-C(O)N(R)₂.
1473. The agent of any one of Embodiments 1468-1472, wherein R is hydrogen or optionally substituted C₁₋₁₀ aliphatic.
1474. The agent of any one of Embodiments 1468-1472, wherein R is hydrogen.
1475. The agent of any one of Embodiments 1468-1472, wherein R is optionally substituted C₁₋₁₀ aliphatic.
1476. The agent of any one of Embodiments 1468-1472, wherein R is C₁₋₁₀ aliphatic.
1477. The agent of any one of Embodiments 1468-1472, wherein R is C₁₋₁₀ alkyl.
1478. The agent of any one of Embodiments 1462-1467, wherein R^{a2} is -L″-OH.
1479. The agent of any one of Embodiments 1462-1478, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1480. The agent of any one of Embodiments 1462-1478, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1481. The agent of any one of Embodiments 1462-1478, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1482. The agent of any one of Embodiments 1462-1478, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1483. The agent of any one of Embodiments 1462-1478, wherein L^{s1} is —(CH₂)n- wherein n is 1, 2, 3, 4, 5, or 6.
1484. The agent of any one of Embodiments 1-1460, wherein X¹⁷ is Ala, Leu, GlnR, GlnR, Pro, Thr, Val, Lys, Arg, [Ac] Lys, [mPEG4]Lys, [mPEG8]Lys, or [mPEG16]Lys.
1485. The agent of any one of Embodiments 1-1460, wherein X¹⁷ is selected from Ala and Leu.
1486. The agent of any one of the preceding Embodiments, wherein X¹⁷ comprises a C-terminal group.
1487. The agent of any one of Embodiments 1-1460, wherein p17 is 0.
1488. The agent of any one of the preceding Embodiments, wherein p18 is 1.
1489. The agent of any one of the preceding Embodiments, wherein X¹⁸ is —N(R^{a1})-L^{a1}-C(R^{a2})(R^{a3})-L^{a2}-C(O)—.
1490. The agent of Embodiment 1489, wherein R^{a1} is —H.
1491. The agent of any one of Embodiments 1489-1490, wherein R^{a3} is —H.
1492. The agent of any one of Embodiments 1489-1490, wherein R^{a3} is optionally substituted C₁₋₆ aliphatic.
1493. The agent of any one of Embodiments 1489-1492, wherein L^{a1} is a covalent bond.
1494. The agent of any one of Embodiments 1489-1493, wherein L^{a2} is a covalent bond.
1495. The agent of any one of Embodiments 1489-1494, wherein R^{a2} is -L″-R.
1496. The agent of any one of Embodiments 1489-1494, wherein R^{a2} is -L″-Cy-R.
1497. The agent of any one of Embodiments 1489-1494, wherein R^{a2} is -L″-C(O)OR.
1498. The agent of any one of Embodiments 1489-1494, wherein R^{a2} is -L″-C(O)N(R′)₂.
1499. The agent of any one of Embodiments 1489-1494, wherein R^{a2} is -L″-C(O)N(R)₂.
1500. The agent of any one of Embodiments 1495-1499, wherein R is hydrogen or optionally substituted C₁₋₁₀ aliphatic.
1501. The agent of any one of Embodiments 1495-1499, wherein R is hydrogen.
1502. The agent of any one of Embodiments 1495-1499, wherein R is optionally substituted C₁₋₁₀ aliphatic.
1503. The agent of any one of Embodiments 1495-1499, wherein R is C₁₋₁₀ aliphatic.
1504. The agent of any one of Embodiments 1495-1499, wherein R is C₁₋₁₀ alkyl.
1505. The agent of any one of Embodiments 1489-1494, wherein R^{a2} is -L″-OH.
1506. The agent of any one of Embodiments 1489-1505, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1507. The agent of any one of Embodiments 1489-1505, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1508. The agent of any one of Embodiments 1489-1505, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1509. The agent of any one of Embodiments 1489-1505, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1510. The agent of any one of Embodiments 1489-1505, wherein L^{s1} is —(CH₂)n- wherein n is 1, 2, 3, 4, 5, or 6.
1511. The agent of any one of Embodiments 1-1487, wherein X^{1′} is Ala, Pro, Leu, [Ac] Lys, [mPEG8]Lys, [mPEG4]Lys, [mPEG16]Lys, Thr, [mPEG37]Lys, [PEG4triPEG16]Lys, [PEG4triPEG36]Lys, or GlnR.
1512. The agent of any one of Embodiments 1-1487, wherein X¹⁸ is Ala.
1513. The agent of any one of the preceding Embodiments, wherein X¹⁸ comprises a C-terminal group.
1514. The agent of any one of Embodiments 1-1487, wherein p18 is 0.
1515. The agent of any one of the preceding Embodiments, wherein p19 is 1.
1516. The agent of any one of the preceding Embodiments, wherein X¹⁹ is —N(R^{a1})-L^{a1}-C(R^{a2})(R^{a3})-L^{a2}-C(O)—.
1517. The agent of Embodiment 1516, wherein R^{a1} is —H.
1518. The agent of any one of Embodiments 1516-1517, wherein R^{a3} is —H.
1519. The agent of any one of Embodiments 1516-1517, wherein R^{a3} is optionally substituted C₁₋₆ aliphatic.
1520. The agent of any one of Embodiments 1516-1519, wherein L^{a1} is a covalent bond.
1521. The agent of any one of Embodiments 1516-1520, wherein L^{a2} is a covalent bond.
1522. The agent of any one of Embodiments 1516-1521, wherein R^{a2} is -L″-R.
1523. The agent of any one of Embodiments 1516-1521, wherein R^{a2} is -L″-Cy-R.
1524. The agent of any one of Embodiments 1516-1521, wherein R^{a2} is -L″-C(O)OR.
1525. The agent of any one of Embodiments 1516-1521, wherein R^{a2} is -L″-C(O)N(R′)₂.
1526. The agent of any one of Embodiments 1516-1521, wherein R^{a2} is -L″-C(O)N(R)₂.
1527. The agent of any one of Embodiments 1522-1526, wherein R is hydrogen or optionally substituted C₁₋₁₀ aliphatic.
1528. The agent of any one of Embodiments 1522-1526, wherein R is hydrogen.
1529. The agent of any one of Embodiments 1522-1526, wherein R is optionally substituted C₁₋₁₀ aliphatic.
1530. The agent of any one of Embodiments 1522-1526, wherein R is C₁₋₁₀ aliphatic.
1531. The agent of any one of Embodiments 1522-1526, wherein R is C₁₋₁₀ alkyl.
1532. The agent of any one of Embodiments 1516-1521, wherein R^{a2} is -L″-OH.
1533. The agent of any one of Embodiments 1516-1532, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1534. The agent of any one of Embodiments 1516-1532, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1535. The agent of any one of Embodiments 1516-1532, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1536. The agent of any one of Embodiments 1516-1532, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1537. The agent of any one of Embodiments 1516-1532, wherein L^{s1} is —(CH₂)n- wherein n is 1, 2, 3, 4, 5, or 6.
1538. The agent of any one of Embodiments 1-1514, wherein X¹⁹ is Ala, Leu, Thr, Val, or Pro.
1539. The agent of any one of Embodiments 1-1487, wherein X¹⁹ is Ala.
1540. The agent of any one of the preceding Embodiments, wherein X¹⁹ comprises a C-terminal group.
1541. The agent of any one of Embodiments 1-1487, wherein p19 is 0.
1542. The agent of any one of the preceding Embodiments, wherein p20 is 1.
1543. The agent of any one of the preceding Embodiments, wherein X²⁰ is —N(R^{a1})-L^{a1}-C(R^{a2})(R^{a3})-L^{a2}-C(O)—.
1544. The agent of Embodiment 1543, wherein R^{a1} is —H.
1545. The agent of any one of Embodiments 1543-1544, wherein R^{a3} is —H.
1546. The agent of any one of Embodiments 1543-1544, wherein R^{a3} is optionally substituted C₁₋₆ aliphatic.
1547. The agent of any one of Embodiments 1543-1546, wherein L^{a1} is a covalent bond.
1548. The agent of any one of Embodiments 1543-1547, wherein L^{a2} is a covalent bond.
1549. The agent of any one of Embodiments 1543-1548, wherein R^{a2} is -L″-R.
1550. The agent of any one of Embodiments 1543-1548, wherein R^{a2} is -L″-Cy-R.
1551. The agent of any one of Embodiments 1543-1548, wherein R^{a2} is -L″-C(O)OR.
1552. The agent of any one of Embodiments 1543-1548, wherein R^{a2} is -L″-C(O)N(R′)₂.
1553. The agent of any one of Embodiments 1543-1548, wherein R^{a2} is -L″-C(O)N(R)₂.
1554. The agent of any one of Embodiments 1549-1553, wherein R is hydrogen or optionally substituted C₁₋₁₀ aliphatic.
1555. The agent of any one of Embodiments 1549-1553, wherein R is hydrogen.
1556. The agent of any one of Embodiments 1549-1553, wherein R is optionally substituted C₁₋₁₀ aliphatic.
1557. The agent of any one of Embodiments 1549-1553, wherein R is C₁₋₁₀ aliphatic.
1558. The agent of any one of Embodiments 1549-1553, wherein R is C₁₋₁₀ alkyl.
1559. The agent of any one of Embodiments 1543-1548, wherein R^{a2} is -L″-OH.
1560. The agent of any one of Embodiments 1543-1559, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1561. The agent of any one of Embodiments 1543-1559, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1562. The agent of any one of Embodiments 1543-1559, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1563. The agent of any one of Embodiments 1543-1559, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1564. The agent of any one of Embodiments 1543-1559, wherein L^{s1} is —(CH₂)n- wherein n is 1, 2, 3, 4, 5, or 6.
1565. The agent of any one of Embodiments 1-1541, wherein X²⁰ is Ala, Leu, Lys, nLeu, Val, or Arg.
1566. The agent of any one of Embodiments 1-1541, wherein X²⁰ is Ala.
1567. The agent of any one of the preceding Embodiments, wherein X²⁰ comprises a C-terminal group.
1568. The agent of any one of Embodiments 1-1541, wherein p20 is 0.
1569. The agent of any one of the preceding Embodiments, wherein p21 is 1.
1570. The agent of any one of the preceding Embodiments, wherein X²¹ is —N(R^{a1})-L^{a1}-C(R^{a2})(R^{a3})-L^{a2}-C(O)—.
1571. The agent of Embodiment 1570, wherein R^{a1} is —H.
1572. The agent of any one of Embodiments 1570-1571, wherein R^{a3} is —H.
1573. The agent of any one of Embodiments 1570-1571, wherein R^{a3} is optionally substituted C₁₋₆ aliphatic.
1574. The agent of any one of Embodiments 1570-1573, wherein L^{a1} is a covalent bond.
1575. The agent of any one of Embodiments 1570-1574, wherein L^{a2} is a covalent bond.
1576. The agent of any one of Embodiments 1570-1575, wherein R^{a2} is -L″-R.
1577. The agent of any one of Embodiments 1570-1575, wherein R^{a2} is -L″-Cy-R.
1578. The agent of any one of Embodiments 1570-1575, wherein R^{a2} is -L″-C(O)OR.
1579. The agent of any one of Embodiments 1570-1575, wherein R^{a2} is -L″-C(O)N(R′)₂.
1580. The agent of any one of Embodiments 1570-1575, wherein R^{a2} is -L″-C(O)N(R)₂.
1581. The agent of any one of Embodiments 1576-1580, wherein R is hydrogen or optionally substituted C₁₋₁₀ aliphatic.
1582. The agent of any one of Embodiments 1576-1580, wherein R is hydrogen.
1583. The agent of any one of Embodiments 1576-1580, wherein R is optionally substituted C₁₋₁₀ aliphatic.
1584. The agent of any one of Embodiments 1576-1580, wherein R is C₁₋₁₀ aliphatic.
1585. The agent of any one of Embodiments 1576-1580, wherein R is C₁₋₁₀ alkyl.
1586. The agent of any one of Embodiments 1570-1575, wherein R^{a2} is -L″-OH.
1587. The agent of any one of Embodiments 1570-1586, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1588. The agent of any one of Embodiments 1570-1586, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1589. The agent of any one of Embodiments 1570-1586, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1590. The agent of any one of Embodiments 1570-1586, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1591. The agent of any one of Embodiments 1570-1586, wherein L^{s1} is —(CH₂)n- wherein n is 1, 2, 3, 4, 5, or 6.
1592. The agent of any one of Embodiments 1-1568, wherein X²¹ is Ala, Leu, Lys, nLeu, Val, or Arg.
1593. The agent of any one of Embodiments 1-1568, wherein X²¹ is Ala.
1594. The agent of any one of the preceding Embodiments, wherein X²¹ comprises a C-terminal group.
1595. The agent of any one of Embodiments 1-1568, wherein p21 is 0.
1596. The agent of any one of the preceding Embodiments, wherein p22 is 1.
1597. The agent of any one of the preceding Embodiments, wherein X²² is —N(R^{a1})-L^{a1}-C(R^{a2})(R^{a3})-L^{a2}-C(O)—.
1598. The agent of Embodiment 1597, wherein R^{a1} is —H.
1599. The agent of any one of Embodiments 1597-1598, wherein R^{a3} is —H.
1600. The agent of any one of Embodiments 1597-1598, wherein R^{a3} is optionally substituted C₁₋₆ aliphatic.
1601. The agent of any one of Embodiments 1597-1600, wherein Lai is a covalent bond.
1602. The agent of any one of Embodiments 1597-1601, wherein L^{a2} is a covalent bond.
1603. The agent of any one of Embodiments 1597-1602, wherein R^{a2} is -L″-R.
1604. The agent of any one of Embodiments 1597-1602, wherein R^{a2} is -L″-Cy-R.
1605. The agent of any one of Embodiments 1597-1602, wherein R^{a2} is -L″-C(O)OR.
1606. The agent of any one of Embodiments 1597-1602, wherein R^{a2} is -L″-C(O)N(R′)₂.
1607. The agent of any one of Embodiments 1597-1602, wherein R^{a2} is -L″-C(O)N(R)₂.
1608. The agent of any one of Embodiments 1603-1607, wherein R is hydrogen or optionally substituted C₁₋₁₀ aliphatic.
1609. The agent of any one of Embodiments 1603-1607, wherein R is hydrogen.
1610. The agent of any one of Embodiments 1603-1607, wherein R is optionally substituted C₁₋₁₀ aliphatic.
1611. The agent of any one of Embodiments 1603-1607, wherein R is C₁₋₁₀ aliphatic.
1612. The agent of any one of Embodiments 1603-1607, wherein R is C₁₋ₙ alkyl.
1613. The agent of any one of Embodiments 1597-1602, wherein R^{a2} is -L″-OH.
1614. The agent of any one of Embodiments 1597-1613, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1615. The agent of any one of Embodiments 1597-1613, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1616. The agent of any one of Embodiments 1597-1613, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1617. The agent of any one of Embodiments 1597-1613, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1618. The agent of any one of Embodiments 1597-1613, wherein L^{s1} is —(CH₂)n- wherein n is 1, 2, 3, 4, 5, or 6.
1619. The agent of any one of Embodiments 1-1595, wherein X²² is Lys.
1620. The agent of any one of the preceding Embodiments, wherein X²² comprises a C-terminal group.
1621. The agent of any one of Embodiments 1-1595, wherein p22 is 0.
1622. The agent of any one of the preceding Embodiments, wherein p23 is 1.
1623. The agent of any one of the preceding Embodiments, wherein X²³ is —N(R^{a1})-L^{a1}-C(R^{a2})(R^{a3})-L^{a2}-C(O)—.
1624. The agent of Embodiment 1623, wherein R^{a1} is —H.
1625. The agent of any one of Embodiments 1623-1624, wherein R^{a3} is —H.
1626. The agent of any one of Embodiments 1623-1624, wherein R^{a3} is optionally substituted C₁₋₆ aliphatic.
1627. The agent of any one of Embodiments 1623-1626, wherein L^{a1} is a covalent bond.
1628. The agent of any one of Embodiments 1623-1627, wherein L^{a2} is a covalent bond.
1629. The agent of any one of Embodiments 1623-1628, wherein R^{a2} is -L″-R.
1630. The agent of any one of Embodiments 1623-1628, wherein R^{a2} is -L″-Cy-R.
1631. The agent of any one of Embodiments 1623-1628, wherein R^{a2} is -L″-C(O)OR.
1632. The agent of any one of Embodiments 1623-1628, wherein R^{a2} is -L″-C(O)N(R′)₂.
1633. The agent of any one of Embodiments 1623-1628, wherein R^{a2} is -L″-C(O)N(R)₂.
1634. The agent of any one of Embodiments 1629-1633, wherein R is hydrogen or optionally substituted C₁₋₁₀ aliphatic.
1635. The agent of any one of Embodiments 1629-1633, wherein R is hydrogen.
1636. The agent of any one of Embodiments 1629-1633, wherein R is optionally substituted C₁₋₁₀ aliphatic.
1637. The agent of any one of Embodiments 1629-1633, wherein R is C₁₋₁₀ aliphatic.
1638. The agent of any one of Embodiments 1629-1633, wherein R is C₁₋₁₀ alkyl.
1639. The agent of any one of Embodiments 1623-1628, wherein R^{a2} is -L″-OH.
1640. The agent of any one of Embodiments 1623-1639, wherein L^{s1} is a covalent bond or an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1641. The agent of any one of Embodiments 1623-1639, wherein L^{s1} is an optionally substituted bivalent C₁₋₁₀ aliphatic wherein one or more methylene units are optionally and independently replaced with —O—, —S—, -Cy-, —N(R′)—, —C(O)—, —C(O)N(R′)—, or —N(R′)C(O)O—.
1642. The agent of any one of Embodiments 1623-1639, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1643. The agent of any one of Embodiments 1623-1639, wherein L^{s1} is a bivalent C₁₋₆ aliphatic wherein one or more methylene units are optionally and independently replaced with —O—, —S—, —N(R′)—, —C(O)—, —C(O)N(R′)—, or —N(R′)C(O)O—.
1644. The agent of any one of Embodiments 1623-1639, wherein L^{s1} is —(CH₂)n- wherein n is 1, 2, 3, 4, 5, or 6.
1645. The agent of any one of the preceding Embodiments, wherein X²³ comprises a C-terminal group.
1646. The agent of any one of Embodiments 1-1621, wherein p23 is 0.
1647. The agent of any one of the preceding Embodiments, wherein the C-terminal group is R^{C}.
1648. The agent of any one of Embodiments 1-1646, wherein the C-terminal group is —OH.
1649. The agent of any one of Embodiments 1-1646, wherein the C-terminal group is —N(R)₂.
1650. The agent of any one of Embodiments 1-1646, wherein the C-terminal group is —N(R)₂, wherein each R is independently —H or optionally substituted C₁₋₆ aliphatic.
1651. The agent of any one of Embodiments 1-1646, wherein the C-terminal group is —NH₂.
1652. The agent of any one of Embodiments 1-1646, wherein the C-terminal group is —NHMe.
1653. The agent of any one of Embodiments 1-1646, wherein the C-terminal group is —NHEt.
1654. The agent of any one of Embodiments 1-1646, wherein the C-terminal group is Serol.
1655. The agent of any one of Embodiments 1-1646, wherein the C-terminal group is dAlaol.
1656. The agent of any one of the preceding Embodiments, wherein the peptide comprises a hydrocarbon staple.
1657. The agent of any one of the preceding Embodiments, wherein the peptide comprises a non-hydrocarbon staple.
1658. The agent of any one of the preceding Embodiments, wherein the peptide comprises a staple whose chain comprises —N(R′)— or —O—C(O)—N(R′)—.
1659. The agent of any one of the preceding Embodiments, wherein the peptide has the structure of:

R^{N}—[X]ₚ[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇—[X]_{p′}—R^{C},
or a salt thereof, wherein:
   each X is independently an amino acid residue;
   each p and p′ is independently 0-10;
   R^{N} is independently a peptide, an amino protecting group or R′-L^{RN}-;
   R^{C} is independently a peptide, a carboxyl protecting group, -L^{RC}-R′, —O-L^{RC}-R′ or —N(R′)-L^{RC}-R′;
   each of L^{RN} and L^{RC} is independently L;
   each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently —R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
1660. The agent of any one of the preceding Embodiments, wherein p is 0.
1661. The agent of any one of the preceding Embodiments, wherein p is 1-10 (e.g., 1, 2, 3, 4, 5, 6, 7, 8, 9, or 10).
1662. The agent of any one of Embodiments 1-1659, wherein p is 1.
1663. The agent of any one of Embodiments 1-1659, wherein p is 2.
1664. The agent of any one of Embodiments 1-1659, wherein p is 3.
1665. The agent of any one of Embodiments 1-1659, wherein p is 4.
1666. The agent of any one of Embodiments 1-1659, wherein p is 5.
1667. The agent of any one of the preceding Embodiments, wherein p′ is 0.
1668. The agent of any one of Embodiments 1-1666, wherein p′ is 1-10 (e.g., 1, 2, 3, 4, 5, 6, 7, 8, 9, or 10).
1669. The agent of any one of Embodiments 1-1666, wherein p′ is 2.
1670. The agent of any one of Embodiments 1-1666, wherein p′ is 3.
1671. The agent of any one of Embodiments 1-1666, wherein p′ is 4.
1672. The agent of any one of Embodiments 1-1666, wherein p′ is 5.
1673. The agent of any one of the preceding Embodiments, wherein R^{N} is —C(O)R.
1674. The agent of any one of the preceding Embodiments, wherein R^{N} is Ac.
1675. The agent of any one of Embodiments 1-1672, wherein R^{N} is Ac, NPyroR3, 5hexenyl, 4pentenyl, Bua, C3a, Cpc, Cbc, CypCO, Bnc, CF3CO, 2PyCypCO, 4THPCO, Isobutyryl, Ts, 15PyraPy, 2PyBu, 4PymCO, 4PyPrpc, 3IAPAc, 4MePipzPrpC, MePipAc, MeImid4SO2, BzAm20Allyl, Hex, 2PyzCO, 3Phc3, MeOPr, lithocholate, 2FPhc, PhC, MeSO2, Isovaleryl, EtHNCO, TzPyr, 8IAP, 3PydCO, 2PymCO, 5PymCO, 1Imidac, 2F2PyAc, 2IAPAc, 124TriPr, 6QuiAc, 3PyAc, 123TriAc, 1PyrazoleAc, 3PyPrpc, 5PymAc, 1PydoneAc, 124TriAc, Me2NAc, 8QuiSO2, mPEG4, mPEG8, mPEG16 or mPEG24.
1676. The agent of any one of Embodiments 1-1672, wherein R^{N} is 4pentenyl.
1677. The agent of any one of Embodiments 1-1672, wherein R^{N} is 5hexenyl.
1678. The agent of any one of Embodiments 1-1672, wherein R^{N} is BzAm20Allyl.
1679. The agent of any one of the preceding Embodiments, wherein R^{C} is —N(R′)₂.
1680. The agent of any one of the preceding Embodiments, wherein R^{C} is —N(R)₂.
1681. The agent of any one of Embodiments 1-1680, wherein R^{C} is —NH₂.
1682. The agent of any one of Embodiments 1-1680, wherein R^{C} is —NHEt.
1683. The agent of any one of Embodiments 1-1680, wherein R^{C} is -Alaol wherein the amino group of -Alaol is bonded to the last —C(O)— of the peptide backbone

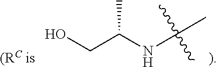 [SMILES: *N[C@@H](C)CO]

1684. The agent of any one of Embodiments 1-1680, wherein R^{C} is -dAlaol, wherein the amino group of -dAlaol is bonded to the last —C(O)— of the peptide backbone

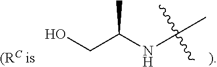 [SMILES: *N[C@H](C)CO]

1685. The agent of any one of Embodiments 1-1680, wherein R^{C} is -Prool, wherein the amino group of -Prool is bonded to the last —C(O)— of the peptide backbone

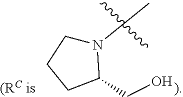 [SMILES: *N1CCC[C@H]1CO]

1686. The agent of any one of Embodiments 1-1680, wherein R^{C} is -Throl, wherein the amino group of -Throl is bonded to the last —C(O)— of the peptide backbone

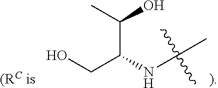 [SMILES: *N[C@H](CO)[C@@H](C)O]

1687. The agent of any one of Embodiments 1-1680, wherein R^{C} is —Serol, wherein the amino group of -Serol is bonded to the last —C(O)— of the peptide backbone

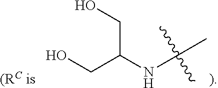 [SMILES: *NC(CO)CO]

1688. The agent of any one of Embodiments 1-1678, wherein R is —OH.
1689. The agent of any one of the preceding Embodiments, wherein each amino acid residue is independently —N(R^{a1})-L^{a1}-C(R^{a2})(R^{a3})-L^{a2}-C(O)—.
1690. The agent of Embodiment 1689, wherein R^{a1} is —H.
1691. The agent of any one of Embodiments 1-1689, wherein R^{a1} are taken together with R^{a2} or R^{a3} and their intervening atom(s) to form an optionally substituted 3-10 (e.g., 3, 4, 5, 6, 7, 8, 9, or 10) membered ring having in addition to the intervening atom(s) 0-5 heteroatoms.
1692. The agent of any one of Embodiments 1-1689, wherein R^{a1} are taken together with R^{a2} or R^{a3} and their intervening atom(s) to form an optionally substituted 5-7 membered ring having in addition to the intervening atom(s) no heteroatoms.
1693. The agent of any one of the preceding Embodiments, wherein L^{a1} is a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
1694. The agent of any one of Embodiments 1-1692, wherein L^{a1} is a covalent bond.
1695. The agent of any one of the preceding Embodiments, wherein R^{a2} is -L^{a}-R′ wherein, L^{a} is a covalent bond or a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
1696. The agent of any one of the preceding Embodiments, wherein R^{a3} is -L^{a}-R′ wherein, L^{a} is a covalent bond or a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
1697. The agent of any one of Embodiments 1-1695, wherein R^{a3} is —H.
1698. The agent of any one of Embodiments 1-1695, wherein R^{a3} is optionally substituted C₁₋₆ aliphatic.
1699. The agent of any one of the preceding Embodiments, wherein L^{a2} is a bivalent C₁₋₆ aliphatic wherein one or more methylene units are optionally and independently replaced with —O—, —S—, —N(R′)—, —C(O)—, -Cy-, —C(O)N(R′)—, or —N(R′)C(O)O—.
1700. The agent of any one of Embodiments 1-1698, wherein L^{a2} is a covalent bond.
1701. The agent of any one of the preceding Embodiments, wherein the agent is or comprises a stapled peptide which comprises a stapled residue at a position referred to as position P.
1702. The agent of Embodiment 1701, wherein the stapled residue at position P is stapled with a residue at position P+7.
1703. The agent of any one of Embodiments 1701-1702, wherein the stapled residue at position P is stapled with a residue at position P-4.
1704. The agent of any one of Embodiments 1701-1702, wherein the stapled residue at position P is stapled with a residue at position P-3.
1705. The agent of any one of Embodiments 1701-1702, wherein the stapled residue at position P is stapled with a residue at position P-2.
1706. The agent of any one of Embodiments 1701-1705, wherein the stapled peptide comprises a staple stapling two residues at positons P+6 and P+10.
1707. The agent of any one of Embodiments 1701-1705, wherein the stapled peptide comprises a staple stapling two residues at positons P+3 and P+10.
1708. The agent of any one of Embodiments 1701-1707, wherein there are three staples in the stapled peptide.
1709. The agent of any one of Embodiments 1701-1707, wherein the stapled peptide comprises a staple stapling two residues at positons P-1 and P+3.
1710. The agent of any one of Embodiments 1701-1707 and 1709, wherein there are four staples in the stapled peptide.
1711. The agent of any one of Embodiments 1701-1710, wherein the stapled peptide comprises an acidic amino acid residue at position P-2.
1712. The agent of any one of Embodiments 1701-1711, wherein the stapled peptide comprises an acidic amino acid residue at position P+1.
1713. The agent of any one of Embodiments 1701-1712, wherein the stapled peptide comprises an acidic amino acid residue at position P+2.
1714. The agent of any one of Embodiments 1701-1713, wherein the stapled peptide comprises a hydrophobic amino acid residue at position P+4.
1715. The agent of any one of Embodiments 1701-1714, wherein the stapled peptide comprises an aromatic amino acid residue at position P+5.
1716. The agent of any one of Embodiments 1701-1715, wherein the stapled peptide comprises an aromatic amino acid residue at position P+8.
1717. The agent of any one of Embodiments 1701-1716, wherein the stapled peptide comprises an aromatic amino acid residue at position P+9.
1718. The agent of any one of Embodiments 1701-1717, wherein position P is position 3.
1719. The agent of any one of Embodiments 1701-1717, wherein position P is position 4.
1720. The agent of any one of Embodiments 1701-1717, wherein position P is position 5.
1721. The agent of any one of Embodiments 1701-1717, wherein position P is position 6.
1722. The agent of any one of Embodiments 1701-1717, wherein position P is position 7.
1723. The agent of any one of the preceding Embodiments, wherein the peptide forms a structure that comprises a helix.
1724. The agent of any one of the preceding Embodiments, wherein the peptide binds to beta-catenin.
1725. The agent of any one of the preceding Embodiments, wherein the peptide binds to beta-catenin with a EC50 of no more than about 2000 nM, or no more than about 1500 nM, or no more than about 1000 nM, or no more than about 500 nM, or no more than about 300 nM, or no more than about 200 nM, or no more than about 100 nM, or no more than about 75 nM, or no more than about 50 nM, or no more than about 25 nM, or no more than about 10 nM as measured by fluorescence polarization.
1726. The agent of any one of the preceding Embodiments, wherein the peptide can compete with TCF7, LEF1, TCF7L1, TCF7L2, Axin1, Axin2, or APC, or a fragment thereof, for beta-catenin binding.
1727. The agent of any one of the preceding Embodiments, wherein the peptide binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

1728. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, R342, K345, V346, V349, Q375, R376, Q379, N380, L382, W383, R386, N387, D413, N415, V416, T418, and C419.
1729. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, R342, K345, V346, V349, Q375, Q379, N380, L382, W383, R386, N387, D413, N415, V416, T418, and C419.
1730. The agent of any one of the preceding Embodiments, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, K345, V346, V349, Q379, N380, L382, W383, R386, N387, D413, N415, V416, T418, and C419.
1731. The agent of any one of the preceding Embodiments, wherein the peptide binds to beta-catenin and interacts with one or more residues that are or correspond to at least two, or at least three, or at least four, or at least five, or at least six, or at least seven of the following amino acid residues in SEQ ID NO: 1 at the indicated positions: G307, K312, K345, W383, N387, D413, and N415.
1732. The agent of any one of the preceding Embodiments, wherein the agent interacts with Y306 of beta-catenin or an amino acid residue corresponding thereto.
1733. The agent of any one of the preceding Embodiments, wherein the agent interacts with G307 of beta-catenin or an amino acid residue corresponding thereto.
1734. The agent of any one of the preceding Embodiments, wherein the agent interacts with K312 of beta-catenin or an amino acid residue corresponding thereto.
1735. The agent of any one of the preceding Embodiments, wherein the agent interacts with K345 of beta-catenin or an amino acid residue corresponding thereto.
1736. The agent of any one of the preceding Embodiments, wherein the agent interacts with Q379 of beta-catenin or an amino acid residue corresponding thereto.
1737. The agent of any one of the preceding Embodiments, wherein the agent interacts with L382 of beta-catenin or an amino acid residue corresponding thereto.
1738. The agent of any one of the preceding Embodiments, wherein the agent interacts with W383 of beta-catenin or an amino acid residue corresponding thereto.
1739. The agent of any one of the preceding Embodiments, wherein the agent interacts with N387 of beta-catenin or an amino acid residue corresponding thereto.
1740. The agent of any one of the preceding Embodiments, wherein the agent interacts with D413 of beta-catenin or an amino acid residue corresponding thereto.
1741. The agent of any one of the preceding Embodiments, wherein the agent interacts with N415 of beta-catenin or an amino acid residue corresponding thereto.
1742. The agent of any one of the preceding Embodiments, wherein the agent interacts with V416 of beta-catenin or an amino acid residue corresponding thereto.
1743. The agent of any one of the preceding Embodiments, wherein the agent binds to beta-catenin at a site that is not an axin binding site.
1744. The agent of any one of the preceding Embodiments, wherein the agent binds to beta-catenin at a site that is not a Bcl9 binding site.
1745. The agent of any one of the preceding Embodiments, wherein the agent binds to beta-catenin at a site that is not a TCF binding site.
1746. The agent of any one of the preceding Embodiments, wherein the agent is the peptide.
1747. An agent having a structure selected from Table E2 or a salt thereof.
1748. An agent having a structure selected from Table E3 or a salt thereof.
1749. An agent, having the structure of

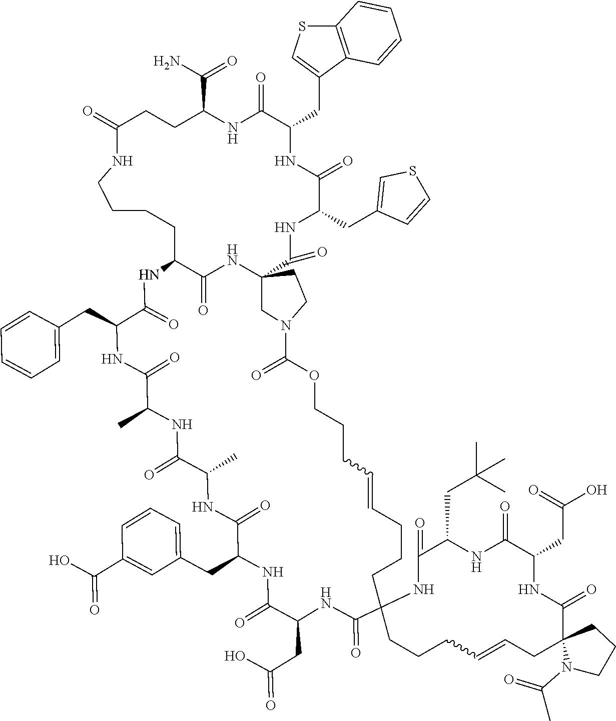 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)[C@H](C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1750. An agent, having the structure of

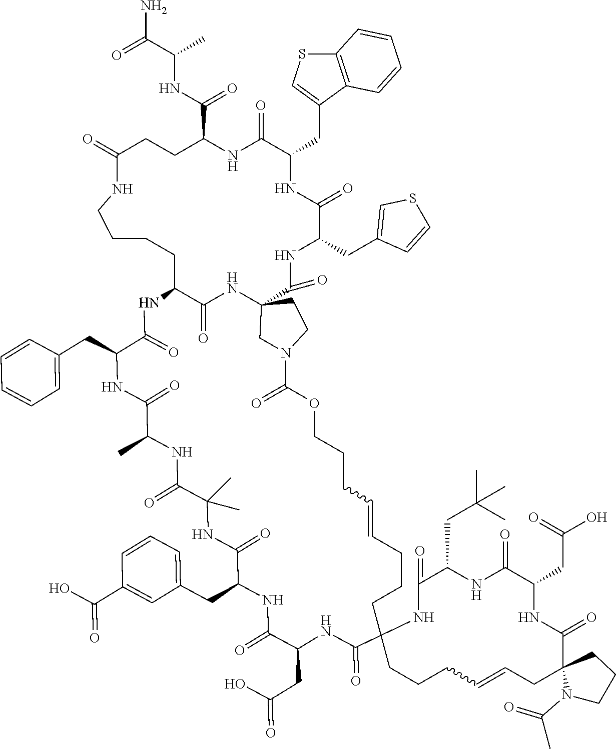 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1751. An agent, having the structure of

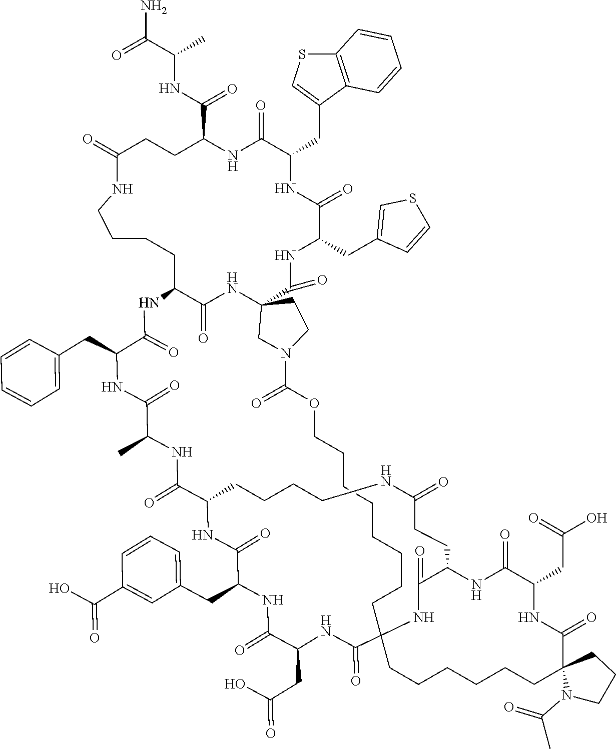 [SMILES: CC(=O)N1CCC[C@]12CCCCCCC13CCCCCCCCOC(=O)N4CC[C@@]5(C4)NC(=O)[C@H](CCCCNC(=O)CC[C@@H](C(=O)N[C@@H](C)C(N)=O)NC(=O)[C@H](Cc4csc6ccccc46)NC(=O)[C@H](Cc4ccsc4)NC5=O)NC(=O)[C@H](Cc4ccccc4)NC(=O)[C@H](C)NC(=O)[C@H](CCCCNC(=O)CC[C@H](NC(=O)[C@H](CC(=O)O)NC2=O)C(=O)N1)NC(=O)[C@H](Cc1cccc(C(=O)O)c1)NC(=O)[C@H](CC(=O)O)NC3=O]

or a salt thereof.
1752. An agent, having the structure of

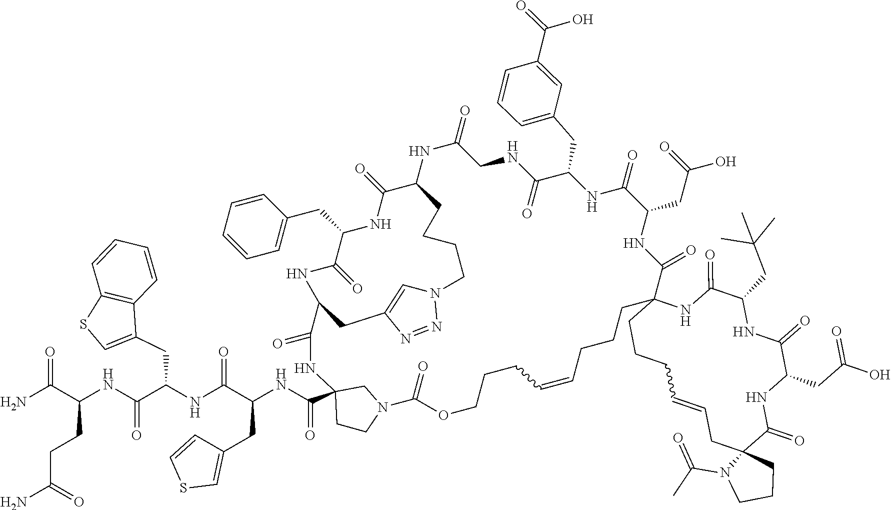

or a salt thereof.
1753. An agent, having the structure of

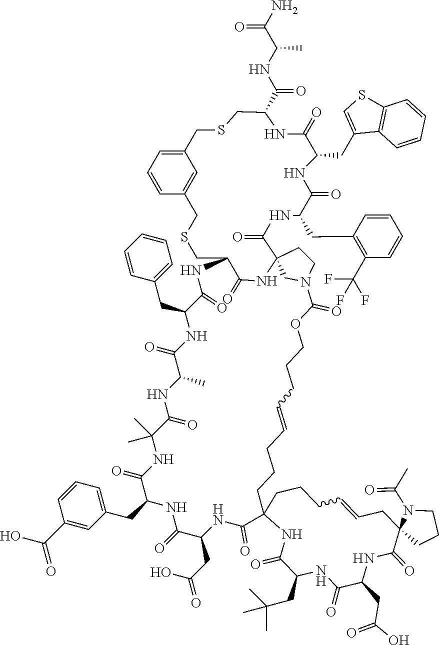 [SMILES: CC(=O)N1CCC[C@]12CC=CCCCC1(CCCC=CCCCOC(=O)N3CC[C@@]4(C3)NC(=O)[C@@H](CSCc3cccc(c3)CSC[C@H](C(=O)N[C@@H](C)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1754. An agent, having the structure of

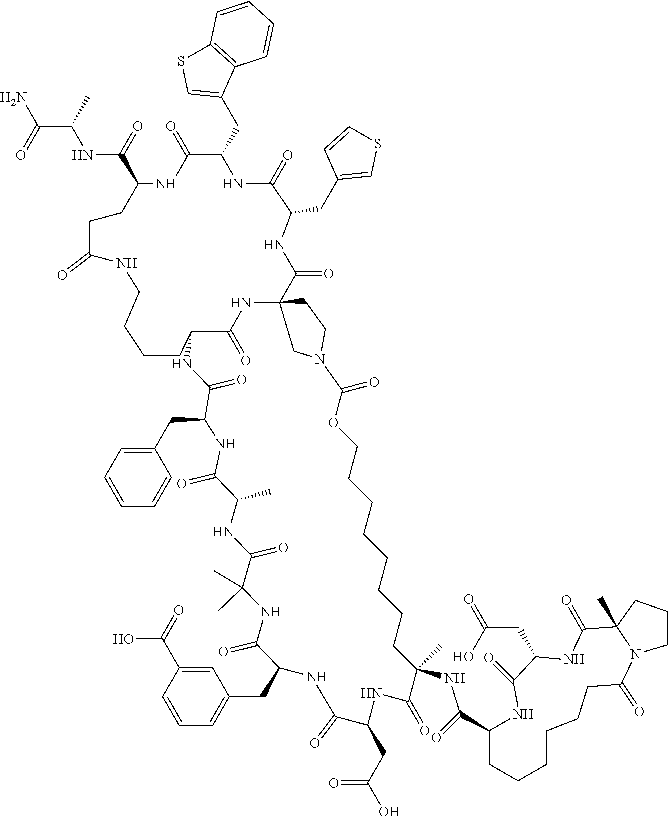 [SMILES: C[C@H](NC(=O)[C@@H]1CCC(=O)NCCCC[C@@H]2NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC(=O)[C@](C)(NC(=O)[C@@H]3CCCCCCC(=O)N4CCC[C@@]4(C)C(=O)N[C@@H](CC(=O)O)C(=O)N3)CCCCCCCCOC(=O)N3CC[C@@](C3)(NC2=O)C(=O)N[C@@H](Cc2ccsc2)C(=O)N[C@@H](Cc2csc3ccccc23)C(=O)N1)C(N)=O]

or a salt thereof. 1755. An agent, having the structure of

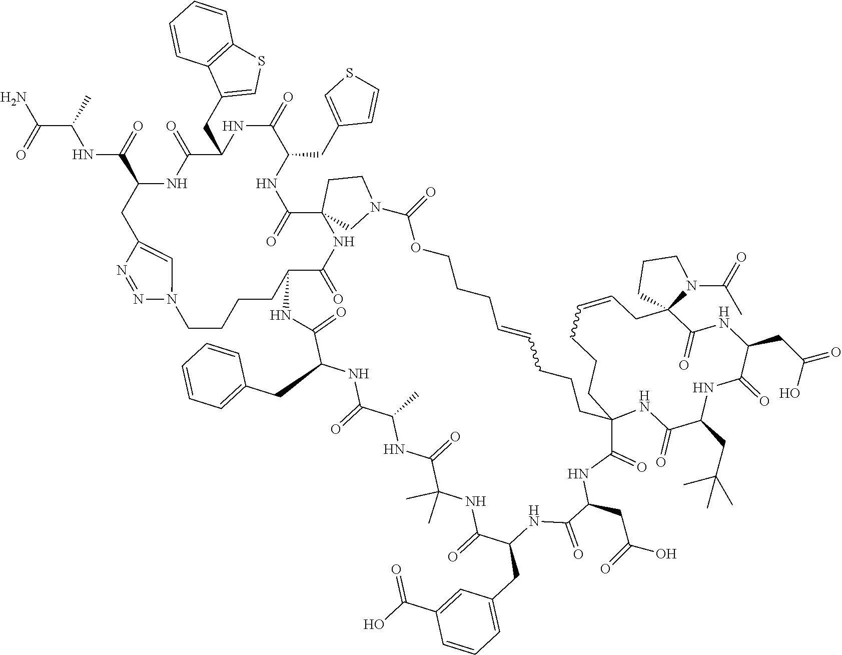 [SMILES: CC(=O)N1CCC[C@]12CC=CCCCC1(CCCC=CCCCOC(=O)N3CC[C@@]4(C3)NC(=O)[C@H](CCCCn3cc(nn3)C[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1756. An agent, having the structure of

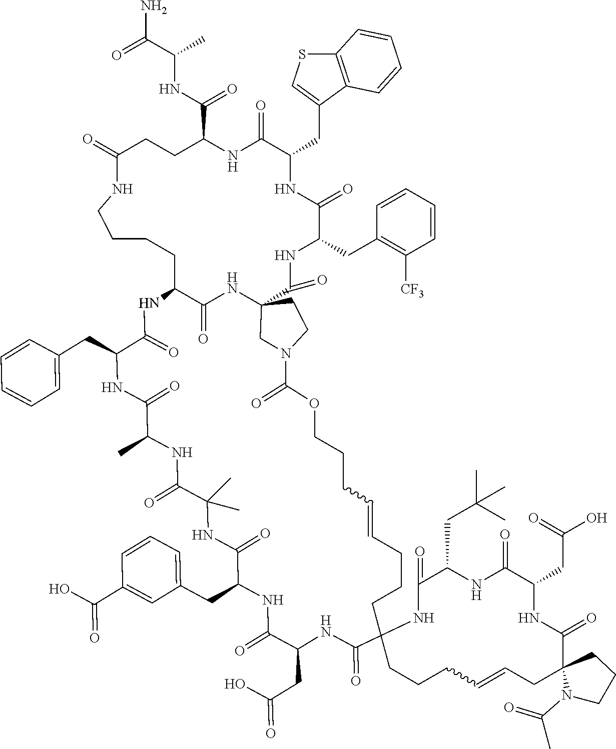 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1757. An agent, having the structure of

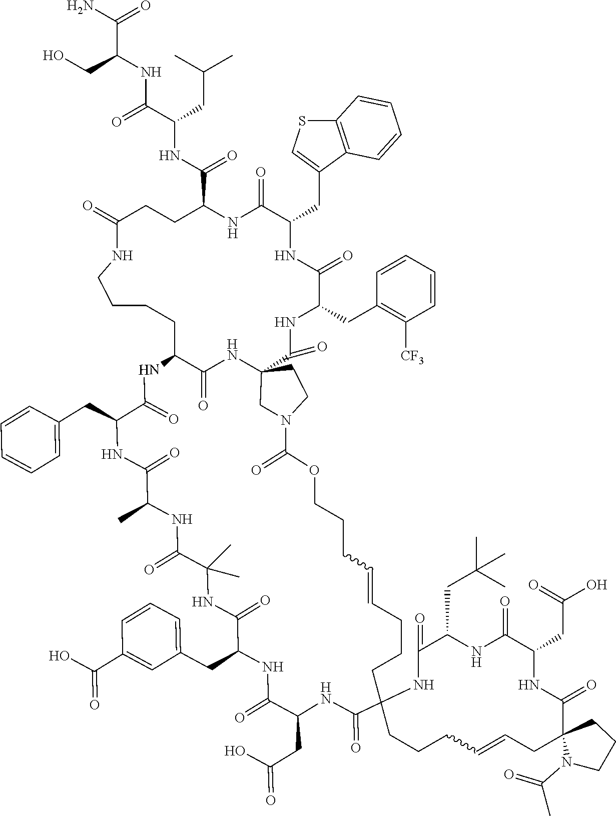 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](CC(C)C)C(=O)N[C@@H](CO)C(N)=O)NC(=O)[C@H](Cc3csc5ccccc35)NC(=O)[C@H](Cc3ccccc3C(F)(F)F)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1758. An agent, having the structure of

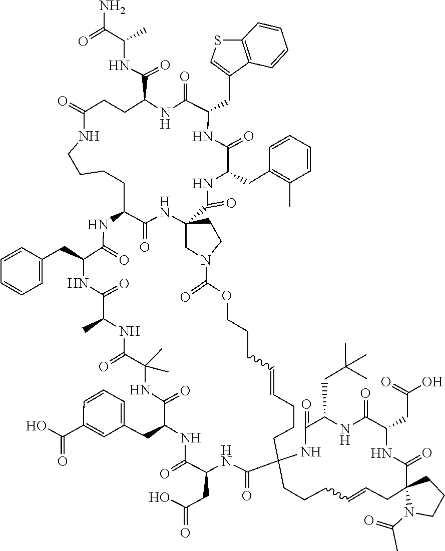 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3C)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1759. An agent, having the structure of

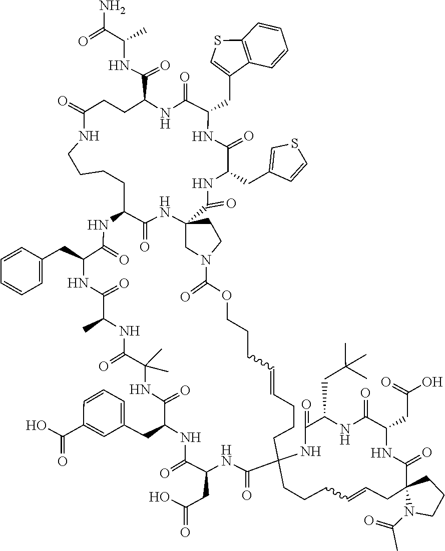 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1760. An agent, having the structure of

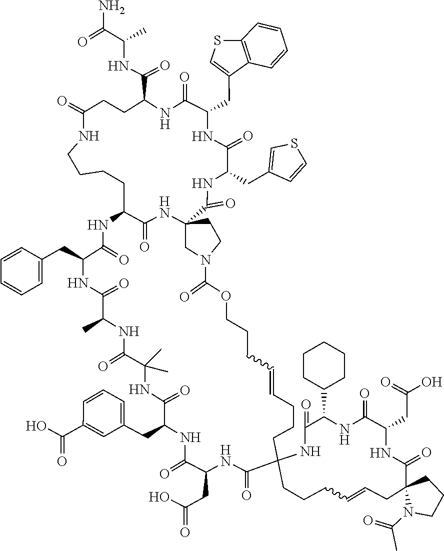 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](C1CCCCC1)NC(=O)[C@H](CC(=O)O)NC2=O]

or a salt thereof.
1761. An agent, having the structure of

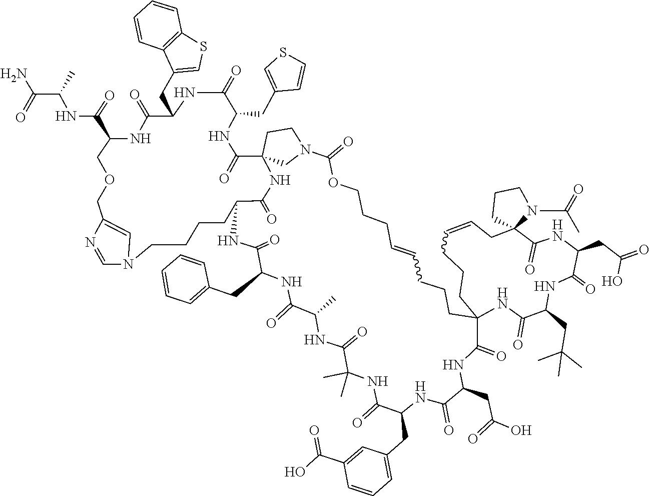

or a salt thereof.
1762. An agent, having the structure of

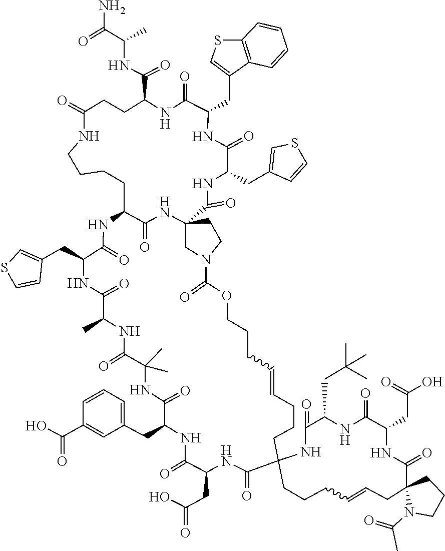 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccsc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1763. An agent, having the structure of

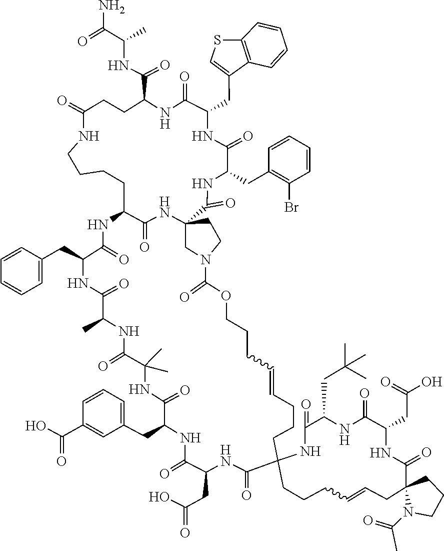 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccccc3Br)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1764. The agent of any one of the preceding Embodiments, wherein a double bond of a staple bonded to the first stapled amino acid that is bonded to a staple with a double bond, counting from the N-terminus, is E.
1765. The agent of any one of the preceding Embodiments, wherein a double bond of a staple bonded to the first stapled amino acid that is bonded to a staple with a double bond, counting from the N-terminus, is Z.
1766. The agent of any one of the preceding Embodiments, wherein a double bond of a staple bonded to the first stapled amino acid that is bonded to a staple with a double bond, counting from the C-terminus, is E.
1767. The agent of any one of the preceding Embodiments, wherein a double bond of a staple bonded to the first stapled amino acid that is bonded to a staple with a double bond, counting from the C-terminus, is Z.
1768. The agent of any one of the preceding Embodiments, wherein a double bond of a (i, i+7) staple is E.
1769. The agent of any one of the preceding Embodiments, wherein a double bond of a (i, i+7) staple is Z.
1770. The agent of any one of the preceding Embodiments, wherein a double bond of a (i, i+2), (i, i+3) or (i, i+4) staple is E.
1771. The agent of any one of the preceding Embodiments, wherein a double bond of a (i, i+2), (i, i+3) or (i, i+4) staple is Z.
1772. The agent of any one of Embodiments 1747-1763, wherein a double bond of a staple bonded to the first amino acid from the N-terminus is Z.
1773. The agent of any one of Embodiments 1747-1771, wherein a double bond of a staple bonded to the 11th amino acid from the N-terminus is E.
1774. The agent of any one of Embodiments 1747-1771, wherein a double bond of a staple bonded to the 11^{th} amino acid from the N-terminus is Z.
1775. The agent of any one of the preceding Embodiments, wherein a carbon atom bonded to two staples (e.g., in B5) is of R configuration.
1776. The agent of any one of any one of the preceding Embodiments, wherein a carbon atom bonded to two staples (e.g., in B5) is of S configuration.
1777. An agent, having the structure of SP-1-1 or a salt thereof.
1778. An agent, having the structure of SP-1-2 or a salt thereof.
1779. An agent, having the structure of SP-1-3 or a salt thereof.
1780. An agent, having the structure of SP-1-4 or a salt thereof.
1781. An agent, having the structure of SP-1-5 or a salt thereof.
1782. An agent, having the structure of SP-1-6 or a salt thereof.
1783. An agent, having the structure of SP-1-7 or a salt thereof.
1784. An agent, having the structure of SP-1-8 or a salt thereof.
1785. An agent, having the structure of SP-2-1 or a salt thereof.
1786. An agent, having the structure of SP-2-2 or a salt thereof.
1787. An agent, having the structure of SP-2-3 or a salt thereof.
1788. An agent, having the structure of SP-2-4 or a salt thereof.
1789. An agent, having the structure of SP-2-5 or a salt thereof.
1790. An agent, having the structure of SP-2-6 or a salt thereof.
1791. An agent, having the structure of SP-2-7 or a salt thereof.
1792. An agent, having the structure of SP-2-8 or a salt thereof.
1793. An agent, having the structure of SP-3-1 or a salt thereof.
1794. An agent, having the structure of SP-3-2 or a salt thereof.
1795. An agent, having the structure of SP-4-1 or a salt thereof.
1796. An agent, having the structure of SP-4-2 or a salt thereof.
1797. An agent, having the structure of SP-4-3 or a salt thereof.
1798. An agent, having the structure of SP-4-4 or a salt thereof.
1799. An agent, having the structure of SP-4-5 or a salt thereof.
1800. An agent, having the structure of SP-4-6 or a salt thereof.
1801. An agent, having the structure of SP-4-7 or a salt thereof.
1802. An agent, having the structure of SP-4-8 or a salt thereof.
1803. An agent, having the structure of SP-5-1 or a salt thereof.
1804. An agent, having the structure of SP-5-2 or a salt thereof.
1805. An agent, having the structure of SP-5-3 or a salt thereof.
1806. An agent, having the structure of SP-5-4 or a salt thereof.
1807. An agent, having the structure of SP-5-5 or a salt thereof.
1808. An agent, having the structure of SP-5-6 or a salt thereof.
1809. An agent, having the structure of SP-5-7 or a salt thereof.
1810. An agent, having the structure of SP-5-8 or a salt thereof.
1811. An agent, having the structure of SP-6 or a salt thereof.
1812. An agent, having the structure of SP-7-1 or a salt thereof.
1813. An agent, having the structure of SP-7-2 or a salt thereof.
1814. An agent, having the structure of SP-7-3 or a salt thereof.
1815. An agent, having the structure of SP-7-4 or a salt thereof.
1816. An agent, having the structure of SP-7-5 or a salt thereof.
1817. An agent, having the structure of SP-7-6 or a salt thereof.
1818. An agent, having the structure of SP-7-7 or a salt thereof.
1819. An agent, having the structure of SP-7-8 or a salt thereof.
1820. An agent, having the structure of SP-8-1 or a salt thereof.
1821. An agent, having the structure of SP-8-2 or a salt thereof.
1822. An agent, having the structure of SP-8-3 or a salt thereof.
1823. An agent, having the structure of SP-8-4 or a salt thereof.
1824. An agent, having the structure of SP-8-5 or a salt thereof.
1825. An agent, having the structure of SP-8-6 or a salt thereof.
1826. An agent, having the structure of SP-8-7 or a salt thereof.
1827. An agent, having the structure of SP-8-8 or a salt thereof.
1828. An agent, having the structure of SP-9-1 or a salt thereof.
1829. An agent, having the structure of SP-9-2 or a salt thereof.
1830. An agent, having the structure of SP-9-3 or a salt thereof.
1831. An agent, having the structure of SP-9-4 or a salt thereof.
1832. An agent, having the structure of SP-9-5 or a salt thereof.
1833. An agent, having the structure of SP-9-6 or a salt thereof.
1834. An agent, having the structure of SP-9-7 or a salt thereof.
1835. An agent, having the structure of SP-9-8 or a salt thereof.
1836. An agent, having the structure of SP-10-1 or a salt thereof.
1837. An agent, having the structure of SP-10-2 or a salt thereof.
1838. An agent, having the structure of SP-10-3 or a salt thereof.
1839. An agent, having the structure of SP-10-4 or a salt thereof.
1840. An agent, having the structure of SP-10-5 or a salt thereof.
1841. An agent, having the structure of SP-10-6 or a salt thereof.
1842. An agent, having the structure of SP-10-7 or a salt thereof.
1843. An agent, having the structure of SP-10-8 or a salt thereof.
1844. An agent, having the structure of SP-11-1 or a salt thereof.
1845. An agent, having the structure of SP-11-2 or a salt thereof.
1846. An agent, having the structure of SP-11-3 or a salt thereof.
1847. An agent, having the structure of SP-11-4 or a salt thereof.
1848. An agent, having the structure of SP-11-5 or a salt thereof.
1849. An agent, having the structure of SP-11-6 or a salt thereof.
1850. An agent, having the structure of SP-11-7 or a salt thereof.
1851. An agent, having the structure of SP-11-8 or a salt thereof.
1852. An agent, having the structure of SP-12-1 or a salt thereof.
1853. An agent, having the structure of SP-12-2 or a salt thereof.
1854. An agent, having the structure of SP-12-3 or a salt thereof.
1855. An agent, having the structure of SP-12-4 or a salt thereof.
1856. An agent, having the structure of SP-12-5 or a salt thereof.
1857. An agent, having the structure of SP-12-6 or a salt thereof.
1858. An agent, having the structure of SP-12-7 or a salt thereof.
1859. An agent, having the structure of SP-12-8 or a salt thereof.
1860. An agent, having the structure of SP-13-1 or a salt thereof.
1861. An agent, having the structure of SP-13-2 or a salt thereof.
1862. An agent, having the structure of SP-13-3 or a salt thereof.
1863. An agent, having the structure of SP-13-4 or a salt thereof.
1864. An agent, having the structure of SP-13-5 or a salt thereof.
1865. An agent, having the structure of SP-13-6 or a salt thereof.
1866. An agent, having the structure of SP-13-7 or a salt thereof.
1867. An agent, having the structure of SP-13-8 or a salt thereof.
1868. An agent, having the structure of SP-14-1 or a salt thereof.
1869. An agent, having the structure of SP-14-2 or a salt thereof.
1870. An agent, having the structure of SP-14-3 or a salt thereof.
1871. An agent, having the structure of SP-14-4 or a salt thereof.
1872. An agent, having the structure of SP-14-5 or a salt thereof.
1873. An agent, having the structure of SP-14-6 or a salt thereof.
1874. An agent, having the structure of SP-14-7 or a salt thereof.
1875. An agent, having the structure of SP-14-8 or a salt thereof.
1876. An agent, having the structure of SP-15-1 or a salt thereof.
1877. An agent, having the structure of SP-15-2 or a salt thereof.
1878. An agent, having the structure of SP-15-3 or a salt thereof.
1879. An agent, having the structure of SP-15-4 or a salt thereof.
1880. An agent, having the structure of SP-15-5 or a salt thereof.
1881. An agent, having the structure of SP-15-6 or a salt thereof.
1882. An agent, having the structure of SP-15-7 or a salt thereof.
1883. An agent, having the structure of SP-15-8 or a salt thereof.
1884. An agent having the structure of

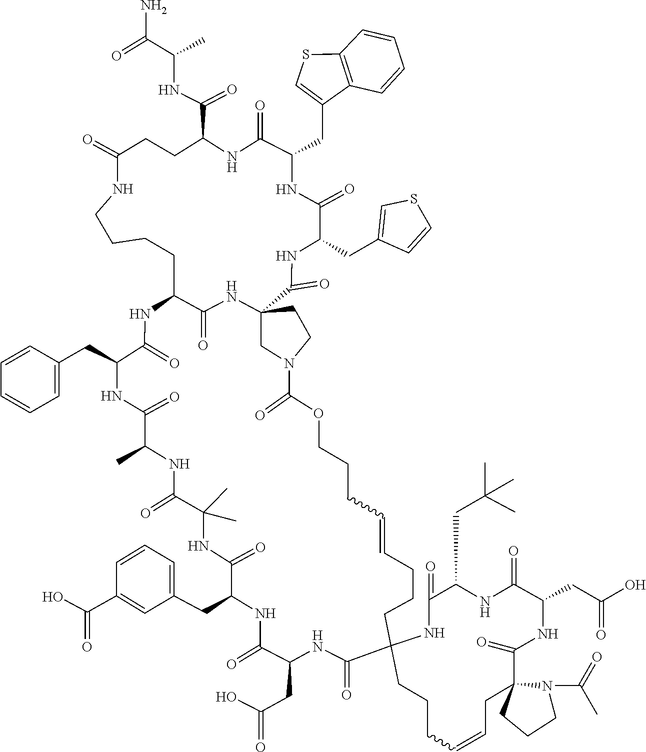 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1885. An agent having the structure of

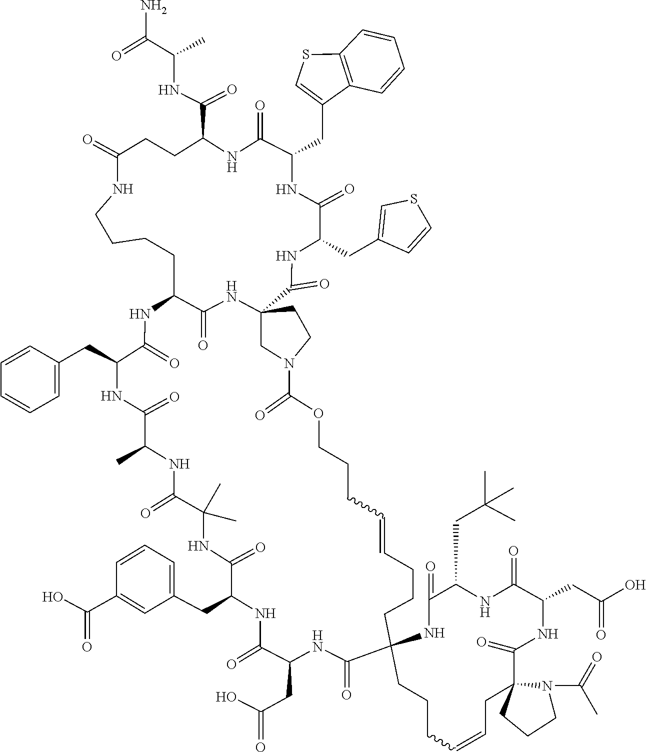 [SMILES: CC(=O)N1CCC[C@]12CC=CCCC[C@]1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof.
1886. An agent having the structure of

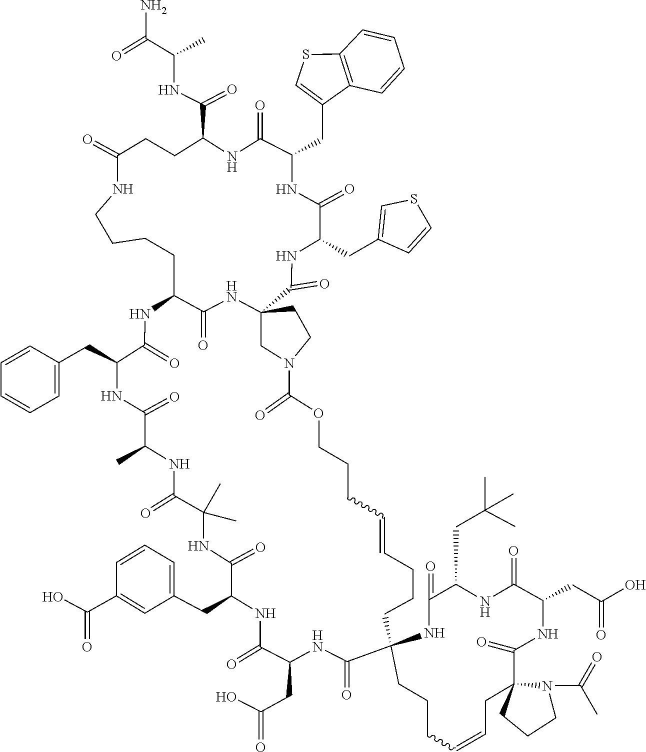 [SMILES: CC(=O)N1CCC[C@]12CC=CCCC[C@]1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

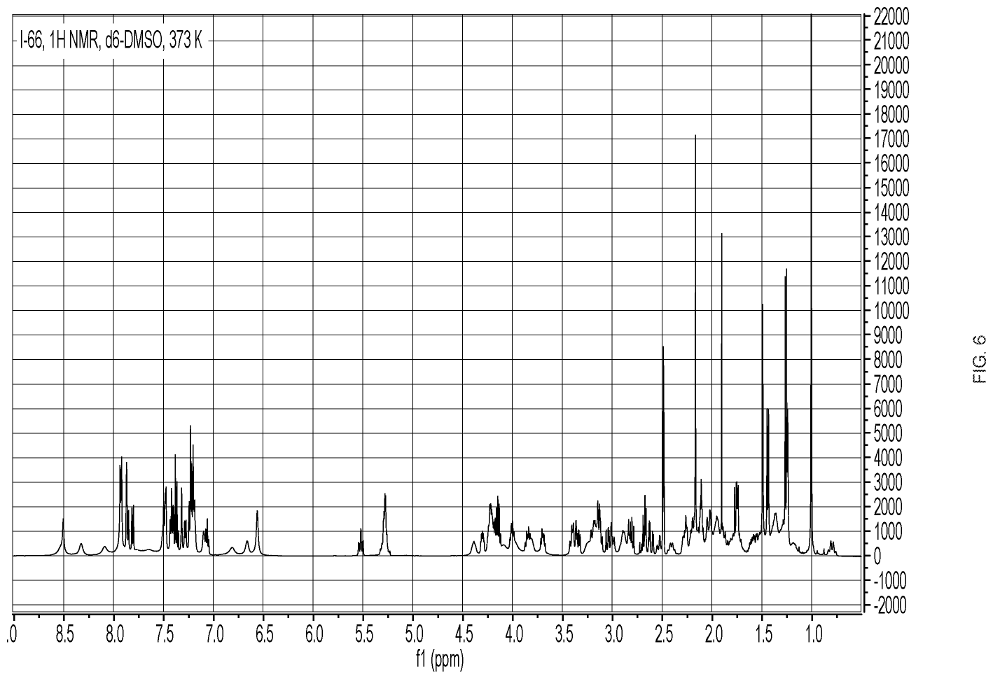

or a salt thereof.
1887. The agent of any one of Embodiments 1884-1886, wherein the agent has the same retention time under a HPLC condition as I-66 prepared as described in Example 9, wherein the HPLC condition can separate I-66 and I-67 prepared as described in Example 9.
1888. The agent of any one of Embodiments 1884-1886, wherein the agent shows a retention time of about 15.3 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.
1889. The agent of any one of Embodiments 1884-1888, wherein the agent elutes in a single peak with I-66 prepared as described in Example 9 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.
1890. The agent of any one of Embodiments 1884-1889, characterized in that the agent shows ¹H NMR peaks that overlap with the peaks between about 5.1-5.7 in FIG. 6 under the same or comparable conditions.
1891. The agent of any one of Embodiments 1884-1889, characterized in that the agent shows the same ¹H NMR peaks between about 5.1-5.7 as FIG. 6 under the same or comparable conditions.
1892. The agent of any one of Embodiments 1884-1889, characterized in that in its ¹H NMR spectrum, the peaks corresponding to ¹H bonded to carbon atoms overlap with peaks in FIG. 6 under the same or comparable conditions.
1893. The agent of any one of Embodiments 1884-1889, characterized in that its ¹H NMR spectrum overlaps with peaks in FIG. 6 under the same or comparable conditions.
1894. The agent of any one of Embodiments 1884-1886, wherein the agent has the same retention time under a HPLC condition as I-67 prepared as described in Example 9, wherein the HPLC condition can separate I-66 and I-67 prepared as described in Example 9.
1895. The agent of any one of Embodiments 1884-1886, wherein the agent shows a retention time of about 16.2 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.
1896. The agent of any one of Embodiments 1884-1888, wherein the agent elutes in a single peak with I-67 prepared as described in Example 9 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.
1897. The agent of any one of Embodiments 1884-1888 and 1894-1896, characterized in that the agent shows ¹H NMR peaks that do not overlap with the peaks between about 5.1-5.7 in FIG. 6 under the same or comparable conditions.
1898. The agent of any one of Embodiments 1884-1888 and 1894-1896, characterized in that the agent does not show the same ¹H NMR peaks between about 5.1-5.7 as FIG. 6 under the same or comparable conditions.
1899. The agent of any one of Embodiments 1884-1888 and 1894-1896, characterized in that in its ¹H NMR spectrum, the peaks corresponding to ¹H bonded to carbon atoms do not all overlap with peaks in FIG. 6 under the same or comparable conditions.
1900. The agent of any one of Embodiments 1884-1889, characterized in that its ¹H NMR spectrum does not overlap with peaks in FIG. 6 under the same or comparable conditions.
1901. A compound having the structure of formula PA:

N(R^{PA})(R^{a1})-L^{a1}-C(R^{a2})(R^{a3})-L^{a2}-C(O)R^{PC}, PA
or a salt thereof, wherein:
   R^{PA} is —H or an amino protecting group;
   each of R^{a1} and R^{a3} is independently -L^{a}-R′; R^{a2} is -L^{aa}-C(O)R^{PS};
   each of L^{a}, Lai and L^{a2} is independently L;
   —C(O)R^{PS} is optionally protected or activated —COOH;
   —C(O)R^{PC} is optionally protected or activated —COOH;
   each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent, 3-30 membered, monocyclic, bicyclic or polycyclic ring having 0-10 heteroatoms;
   each R′ is independently —R, —C(O)R, —CO₂R, or —SO₂R; and
   each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms; or
   two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.
1902. The compound of Embodiment 1901, wherein R^{a2} is -L^{aa}-C(O)R^{P}s, wherein L^{aa} is L and L^{aa} comprises —N(R′)— or -Cy-.
1903. The compound of any one of the preceding Embodiments, wherein L^{a1} is a covalent bond.
1904. The compound of any one of the preceding Embodiments, wherein L^{a2} is a covalent bond.
1905. The compound of any one of the preceding Embodiments, wherein L^{aa} is an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein at least one methylene unit is replaced with -Cy-.
1906. The compound of any one of the preceding Embodiments, wherein L^{aa} is -L^{am1}-Cy-L^{am2}-, wherein each of L^{am1} and L^{a2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
1907. The compound of any one of the preceding Embodiments, wherein -L^{am1}- is bonded to —C(O)R^{PS}.
1908. The compound of any one of the preceding Embodiments, wherein L^{am2} is a covalent bond.
1909. The compound of any one of the preceding Embodiments, wherein -Cy- is an optionally substituted 4-7 membered ring having 0-3 heteroatoms.
1910. The compound of any one of the preceding Embodiments, wherein -Cy- is an optionally substituted 6-10 membered aryl ring or is an optionally substituted 5-10 membered heteroaryl ring having 1-5 heteroatoms.
1911. The compound of any one of the preceding Embodiments, wherein -Cy- is an optionally substituted phenyl ring.
1912. The compound of any one of the preceding Embodiments, wherein -Cy- is optionally substituted

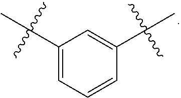 [SMILES: *c1cccc(*)c1]

1913. The compound of any one of the preceding Embodiments, wherein -Cy- is

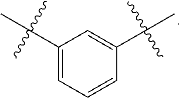 [SMILES: *c1cccc(*)c1]

1914. The compound of any one of Embodiments 1901-1908, wherein -Cy- is optionally substituted

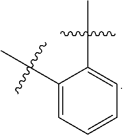 [SMILES: *c1ccccc1*]

1915. The compound of any one of Embodiments 1901-1908, wherein -Cy- is

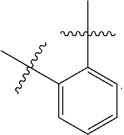 [SMILES: *c1ccccc1*]

1916. The compound of any one of Embodiments 1901-1908, wherein -Cy- is optionally substituted

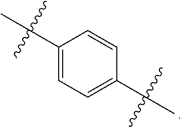 [SMILES: *c1ccc(*)cc1]

1917. The compound of any one of Embodiments 1901-1908, wherein -Cy- is

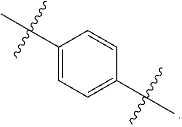 [SMILES: *c1ccc(*)cc1]

1918. The compound of any one of Embodiments 1901-1910, wherein -Cy- is an optionally substituted 5-10 membered heteroaryl ring having 1-5 heteroatoms.
1919. The compound of any one of Embodiments 1901-1910, wherein -Cy- is an optionally substituted 5-membered heteroaryl ring having 1-5 heteroatoms.
1920. The compound of any one of Embodiments 1901-1910, wherein -Cy- is optionally substituted

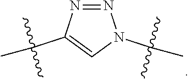 [SMILES: *c1cn(*)nn1]

1921. The compound of any one of Embodiments 1901-1910, wherein -Cy- is

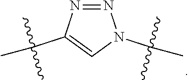 [SMILES: *c1cn(*)nn1]

1922. The compound of any one of the preceding Embodiments, wherein L^{aa} comprises —N(R′)—.
1923. The compound of Embodiment 1922, wherein L^{aa} is -L^{am1}-(NR′)-L^{am2}-, wherein each of L^{am1} and L^{am2} is independently L^{am1}, wherein each L^{am} is independently a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
1924. The compound of any one of Embodiments 1922-1923, wherein R′ of the —N(R′)— is taken together with R^{a3} and their intervening atoms to form an optionally substituted 3-10 membered ring having 0-5 heteroatoms in addition to the intervening atoms.
1925. The compound of any one of Embodiments 1922-1924, wherein —N(R′)— is bonded to two carbon atoms which two carbon atoms do not form any double bonds with heteroatoms.
1926. The compound of any one of Embodiments 1922-1925, wherein -L^{am2} is bonded to —C(O)R^{PS}.
1927. The compound of any one of Embodiments 1922-1926, wherein L^{am1} is optionally substituted C₁₋₄ alkylene.
1928. The compound of any one of Embodiments 1922-1926, wherein L^{am1} is optionally substituted —(CH₂)m-, wherein m is 1, 2, 3, or 4.
1929. The compound of any one of Embodiments 1922-1926, wherein L^{am1} is optionally substituted —CH₂—.
1930. The compound of any one of Embodiments 1922-1926, wherein L^{am1} is —CH₂—.
1931. The compound of any one of Embodiments 1922-1930, wherein L^{am2} is optionally substituted linear C₁₋₂ alkylene.
1932. The compound of any one of Embodiments 1922-1930, wherein L^{am2} is —[C(R′)₂]n, wherein n is 1 or 2.
1933. The compound of any one of Embodiments 1922-1930, wherein L^{am2} is —[CHR′]n, wherein n is 1 or 2.
1934. The compound of any one of Embodiments 1932-1933, wherein each R′ is independently —H or optionally substituted C₁₋₆ alkyl.
1935. The compound of any one of Embodiments 1922-1930, wherein L^{am2} is optionally substituted —CH₂—.
1936. The compound of any one of Embodiments 1922-1935, wherein L^{am2} is —CH₂—.
1937. The compound of any one of Embodiments 1922-1936, wherein L^{aa} comprises —N(R′)—, wherein R′ of the —N(R′)— is —R^{N}, wherein R^{NR} is R.
1938. The compound of any one of Embodiments 1922-1936, wherein L^{aa} comprises —N(R′)—, wherein R′ of the —N(R′)— is —CH₂—R^{NR}, wherein R^{NR} is R.
1939. The compound of any one of Embodiments 1922-1936, wherein L^{aa} comprises —N(R′)—, wherein R′ of the —N(R′)— is —C(O)R^{NR}, wherein R^{NR} is R.
1940. The compound of any one of Embodiments 1922-1936, wherein L^{aa} comprises —N(R′)—, wherein R′ of the —N(R′)— is —SO₂R^{NR}, wherein R^{NR} is R.
1941. The compound of any one of Embodiments 1937-1940, wherein R^{NR} is optionally substituted C₁₋₆ aliphatic or heteroaliphatic having 1-4 heteroatoms.
1942. The compound of any one of Embodiments 1937-1941, wherein R^{NR} is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.
1943. The compound of any one of Embodiments 1937-1942, wherein R^{NR} is —CF₃.
1944. The compound of any one of Embodiments 1937-1941, wherein L^{am2} is or comprises —C(R′)₂— wherein the R′ group and R′ in —N(R′)— of L^{aa} are taken together with their intervening atom(s) to form an optionally substituted, 3-30 membered, monocyclic, bicyclic or polycyclic ring having, in addition to the intervening atom(s), 0-10 heteroatoms.
1945. The compound of any one of Embodiments 1901-1905, wherein L^{aa} is optionally substituted C₁₋₄ alkylene.
1946. The compound of Embodiment 1945, wherein L^{aa} is optionally substituted —CH₂—CH₂—.
1947. The compound of Embodiment 1945, wherein L^{aa} is optionally substituted —CH₂—.
1948. The compound of Embodiment 1901, having the structure of:

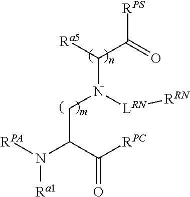

or a salt thereof, wherein:
   each of m and n is independently 1, 2, 3, or 4;
   L^{RN} is L;
   R^{RN} is R; and
   R^{a5} is R′.
1949. The compound of Embodiment 1948, wherein m is 1.
1950. The compound of any one of Embodiments 1948-1949, wherein L^{RN} is —CH₂—, —CO—, or —SO₂—.
1951. The compound of any one of Embodiments 1948-1949, wherein L^{RN} is —CH₂—.
1952. The compound of any one of Embodiments 1948-1951, wherein R^{NR} is C₁₋₇ alkyl or heteroalkyl having 1-4 heteroatoms, wherein the alkyl or heteroalkyl is optionally substituted with one or more groups independently selected from halogen, a C₅₋₆ aromatic ring having 0-4 heteroatoms, and an optionally substituted 3-10 membered cycloalkyl or heteroalkyl ring having 1-4 heteroatoms.
1953. The compound of any one of Embodiments 1948-1952, wherein one or more R^{a5} are independently —H.
1954. The compound of any one of Embodiments 1948-1953, wherein one or more R^{a5} are independently optionally substituted C₁₋₆ alkyl.
1955. The compound of any one of Embodiments 1948-1953, wherein -L^{RN}-R^{RN} is R, and is taken together with a R^{a5} and their intervening atom(s) to form an optionally substituted, 3-30 membered, monocyclic, bicyclic or polycyclic ring having, in addition to the intervening atom(s), 0-10 heteroatoms.
1956. The compound of Embodiment 1951, wherein R^{RN} is methyl.
1957. The compound of Embodiment 1951, wherein R^{RN} is —CF₃.
1958. The compound of any one of the preceding Embodiments, wherein R^{a1} is —H.
1959. The compound of any one of Embodiments 1901-1944, wherein R^{a1} is optionally substituted C₁₋₆ alkyl.
1960. The compound of any one of the preceding Embodiments, wherein —C(O)R^{PC} is a protected carboxylic acid group.
1961. The compound of any one of Embodiments 1901-1959, wherein —C(O)R^{PC} is an activated carboxylic acid group.
1962. The compound of any one of Embodiments 1901-1959, wherein —C(O)R^{PC} is —C(O)OR′.
1963. The compound of Embodiment 1962, wherein R′ is —H.
1964. The compound of Embodiment 1962, wherein R′ is pentafluorophenyl.
1965. The compound of Embodiment 1962, wherein R′ is

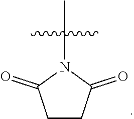 [SMILES: *N1C(=O)CCC1=O]

1966. The compound of any one of the preceding Embodiments, wherein —C(O)R^{PS} is —C(O)OR′.
1967. The compound of Embodiment 1966, wherein R′ is —H.
1968. The compound of Embodiment 1966, wherein R′ is optionally substituted C₁₋₆ aliphatic.
1969. The compound of Embodiment 1966, wherein R′ is t-butyl.
1970. The compound of Embodiment 1966, wherein R′ is benzyl.
1971. The compound of Embodiment 1966, wherein R′ is allyl.
1972. The compound of Embodiment 1901, wherein the compound has the structure of

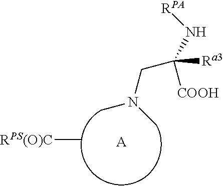

or a salt thereof, wherein Ring A is an optionally substituted 3-7 membered saturated, partially unsaturated or aromatic ring.
1973. The compound of Embodiment 1901, wherein the compound has the structure of

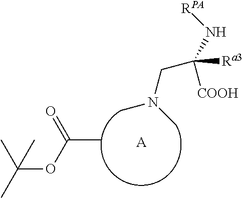

or a salt thereof, wherein Ring A is an optionally substituted 3-7 membered saturated, partially unsaturated or aromatic ring.
1974. The compound of any one of Embodiment 1972 or 1973, wherein —C(O)OtBu is bonded to a chiral carbon atom having a R configuration.
1975. The compound of any one of Embodiment 1972 or 1973, wherein —C(O)OtBu is bonded to a chiral carbon atom having a S configuration.
1976. The compound of Embodiment 1901, wherein the compound has the structure of

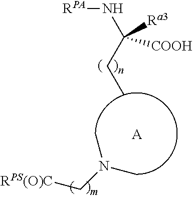

or a salt thereof, wherein:
   Ring A is an optionally substituted 3-10 membered ring;
   n is 0, 1, or 2;
   m is 0, 1, 2, or 3.
1977. The compound of Embodiment 1901, wherein the compound has the structure of

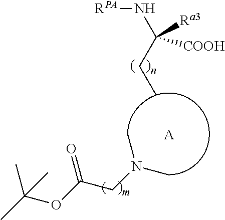

or a salt thereof, wherein:
   Ring A is an optionally substituted 3-10 membered ring;
   n is 0, 1, or 2;
   m is 0, 1, 2, or 3.
1978. The compound of any one of Embodiments 1976-1977, wherein Ring A is an optionally substituted 4-10 membered ring.
1979. The compound of any one of Embodiments 1976-1978, wherein n is 1.
1980. The compound of any one of Embodiments 1976-1979, wherein Ring A is bonded to —(CH₂)n- at a chiral carbon which is R.
1981. The compound of any one of Embodiments 1976-1979, wherein Ring A is bonded to —(CH₂)n- at a chiral carbon which is S.
1982. The compound of Embodiment 1901, wherein the compound has the structure of

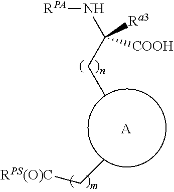

or a salt thereof, wherein:
   Ring A is an optionally substituted 3-10 membered ring;
   n is 0, 1, or 2;
   m is 0, 1, 2, or 3.
1983. The compound of Embodiment 1901, wherein the compound has the structure of

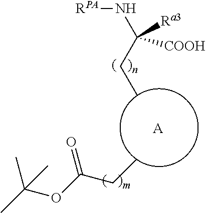

or a salt thereof, wherein:
   Ring A is an optionally substituted 3-10 membered ring;
   n is 0, 1, or 2;
   m is 0, 1, 2, or 3.
1984. The compound of Embodiment 1901, wherein the compound has the structure of

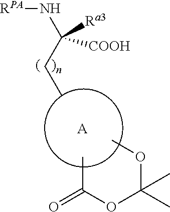

or a salt thereof, wherein:
   Ring A is an optionally substituted 3-10 membered ring;
   n is 0, 1, or 2;
   m is 0, 1, 2, or 3.
1985. The compound of any one of Embodiments 1976-1984, wherein n is 1.
1986. The compound of any one of Embodiments 1976-1985, wherein m is 0.
1987. The compound of any one of Embodiments 1976-1985, wherein m is 1, 2 or 3.
1988. The compound of any one of Embodiments 1976-1985, wherein m is 1.
1989. The compound of any one of Embodiments 1976-1988, wherein Ring A is or comprises an optionally substituted saturated monocyclic ring.
1990. The compound of any one of Embodiments 1976-1989, wherein Ring A is or comprises an optionally substituted partially unsaturated monocyclic ring.
1991. The compound of any one of Embodiments 1976-1990, wherein Ring A is or comprises an optionally substituted aromatic monocyclic ring.
1992. The compound of any one of Embodiments 1982-1988, wherein Ring A is optionally substituted phenyl.
1993. The compound of any one of Embodiments 1976-1988, wherein Ring A is optionally substituted 5-6 membered heteroaryl having 1-3 heteroatoms.
1994. The compound of any one of Embodiments 1976-1988, wherein Ring A is optionally substituted 5-6 membered heteroaryl having 1-3 heteroatoms, wherein at least one heteroatom is nitrogen.
1995. The compound of Embodiment 1994, wherein Ring A is an optionally substituted triazole ring.
1996. The compound of any one of Embodiments 1976-1988, wherein Ring A is an optionally substituted 8-10 membered bicyclic ring having 1-6 heteroatoms.
1997. The compound of any one of Embodiments 1976-1979, wherein Ring A is an optionally substituted 8-10 membered bicyclic aromatic ring having 1-6 heteroatoms, wherein each monocyclic unit is independently an optionally 5-6 membered aromatic ring having 0-3 heteroatoms.
1998. The compound of any one of Embodiments 1993-1997, wherein Ring A is bonded to —(CH₂)n- at a carbon atom.
1999. The compound of any one of Embodiments 1993-1997, wherein Ring A is bonded to —(CH₂)n- at a nitrogen atom.
2000. The compound of any one of the preceding Embodiments, wherein Ring A or -Cy- in L^{aa} is optionally substituted, and each substitute is independently selected from halogen, —R, —CF₃, —N(R)₂, —CN, and —OR, wherein each R is independently C₁₋₆ aliphatic optionally substituted with one or more —F.
2001. The compound of any one of the preceding Embodiments, wherein Ring A or -Cy- in L^{aa} is optionally substituted, and each substitute is independently selected from halogen, C₁₋₅ linear, branched or cyclic alkyl, —OR wherein R is C₁₋₄ linear, branched or cyclic alkyl, fluorinated alkyl, —N(R)₂ wherein each R is independently C₁₋₆ linear, branched or cyclic alkyl, or —CN.
2002. The compound of any one of the preceding Embodiments, wherein R^{a3} is —H or optionally substituted C₁₋₆ aliphatic.
2003. The compound of any one of the preceding Embodiments, wherein R^{a3} is —H.
2004. The compound of any one of Embodiments 1901-2002, wherein R^{a3} is methyl.
2005. A compound having the structure of:

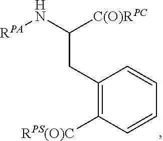

or a salt thereof, wherein:
   R^{PA} is —H or an amino protecting group;
   —C(O)R^{PS} is optionally protected or activated —COOH; and
   —C(O)R^{PC} is optionally protected or activated —COOH.
2006. A compound having the structure of:

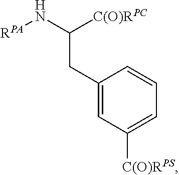

or a salt thereof, wherein:
   R^{PA} is —H or an amino protecting group;
   —C(O)R^{PS} is optionally protected or activated —COOH; and
   —C(O)R^{PC} is optionally protected or activated —COOH.
2007. The compound of any one of the preceding Embodiments, wherein R^{PA} is an amino protecting group suitable for peptide synthesis.
2008. The compound of any one of the preceding Embodiments, wherein R^{PA} is —C(O)—O—R.
2009. The compound of Embodiment 2008, wherein R is optionally substituted

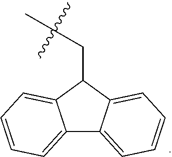 [SMILES: *CC1c2ccccc2-c2ccccc21]

2010. The compound of any one of the preceding Embodiments, wherein R^{PA} is —Fmoc.
2011. The compound of any one of the preceding Embodiments, wherein R^{PA} is —Cbz.
2012. The compound of any one of the preceding Embodiments, wherein R^{PA} is -Boc.
2013. The compound of any one of the preceding Embodiments, wherein R^{PS} is a protecting group orthogonal to R^{PA}.
2014. The compound of any one of the preceding Embodiments, wherein R^{PS} is a protecting group orthogonal to R^{PC}.
2015. The compound of any one of the preceding Embodiments, wherein R^{PS} is compatible with peptide synthesis.
2016. The compound of any one of the preceding Embodiments, wherein —C(O)R^{PS} is —C(O)OR′.
2017. The compound of Embodiment 1966, wherein R′ is —H.
2018. The compound of Embodiment 1966, wherein R′ is optionally substituted C₁₋₆ aliphatic.
2019. The compound of Embodiment 1966, wherein R′ is t-butyl.
2020. The compound of Embodiment 1966, wherein R′ is benzyl.
2021. The compound of Embodiment 1966, wherein R′ is allyl.
2022. The compound of any one of Embodiments 1901-2015, wherein —C(O)R^{PS} is —C(O)S-L-R′.
2023. The compound of Embodiment 2022, wherein L is optionally substituted —CH₂—.
2024. The compound of Embodiment 2022, wherein L is —CH₂—.
2025. The compound of any one of Embodiments 2022-2024, wherein R′ is optionally substituted phenyl.
2026. The compound of any one of Embodiments 2022-2024, wherein R′ is 2, 4, 6-trimethoxyphenyl.
2027. The compound of Embodiment 2022, wherein R^{PS} is —SH.
2028. The compound of any one of the preceding Embodiments, wherein —C(O)R^{PC} is a protected carboxylic acid group.
2029. The compound of any one of Embodiments 1901-2026, wherein —C(O)R^{PC} is an activated carboxylic acid group.
2030. The compound of any one of Embodiments 1901-2026, wherein —C(O)R^{PC} is —C(O)OR′.
2031. The compound of Embodiment 2030, wherein R′ is —H.
2032. The compound of Embodiment 2030, wherein R′ is pentafluorophenyl.
2033. The compound of Embodiment 2030, wherein R′ is

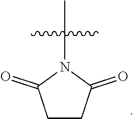 [SMILES: *N1C(=O)CCC1=O]

2034. The compound of any one of the preceding Embodiments, wherein each heteroatom is independently selected from oxygen, nitrogen, sulfur, phosphorus and silicon.
2035. The compound of any one of the preceding Embodiments, wherein each heteroatom is independently selected from oxygen, nitrogen, and sulfur.
2036. A compound, wherein the compound is

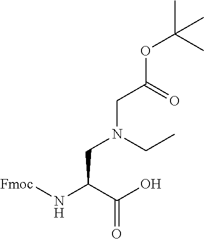

or a salt thereof.
2037. A compound, wherein the compound is

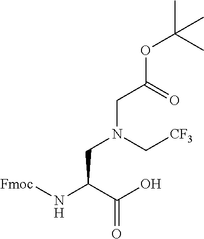

or a salt thereof.
2038. A compound, wherein the compound is

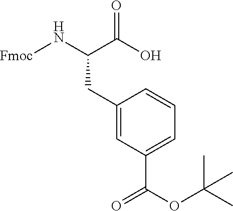

or a salt thereof.
2039. A compound, wherein the compound is

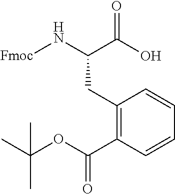

or a salt thereof.
2040. A compound, wherein the compound is

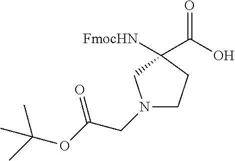

or a salt thereof.
2041. A compound, wherein the compound is

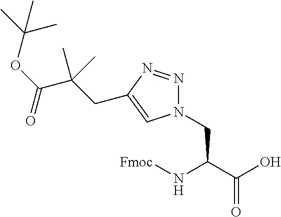

or a salt thereof.
2042. A compound, wherein the compound is

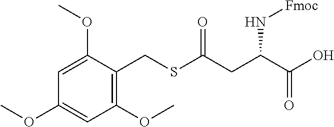

or a salt thereof.
2043. A compound, wherein the compound is

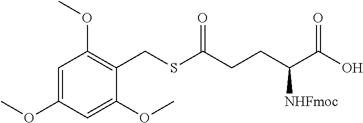

or a salt thereof.
2044. A compound, wherein the compound is

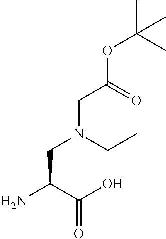 [SMILES: CCN(CC(=O)OC(C)(C)C)C[C@H](N)C(=O)O]

or a salt thereof.
2045. A compound, wherein the compound is

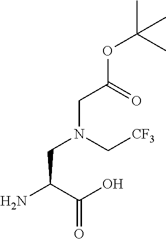 [SMILES: CC(C)(C)OC(=O)CN(C[C@H](N)C(=O)O)CC(F)(F)F]

or a salt thereof.
2046. A compound, wherein the compound is

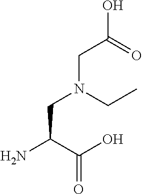

or a salt thereof.
2047. A compound, wherein the compound is

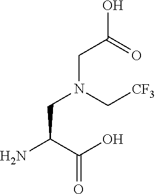 [SMILES: N[C@@H](CN(CC(=O)O)CC(F)(F)F)C(=O)O]

or a salt thereof.
2048. A compound, wherein the compound is

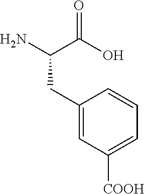 [SMILES: N[C@@H](Cc1cccc(C(=O)O)c1)C(=O)O]

or a salt thereof.
2049. A compound, wherein the compound is

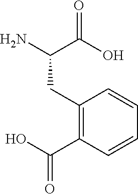 [SMILES: N[C@@H](Cc1ccccc1C(=O)O)C(=O)O]

or a salt thereof.
2050. A compound, wherein the compound is

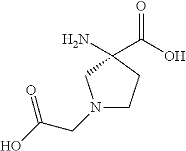 [SMILES: N[C@@]1(C(=O)O)CCN(CC(=O)O)C1]

or a salt thereof.
2051. A compound, wherein the compound is

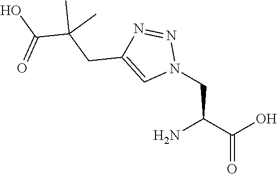 [SMILES: CC(C)(Cc1cn(C[C@H](N)C(=O)O)nn1)C(=O)O]

or a salt thereof.
2052. A compound, wherein the compound is

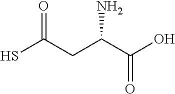

or a salt thereof.
2053. A compound, wherein the compound is

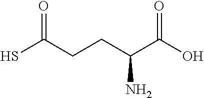

or a salt thereof.
2054. A compound, wherein the compound is

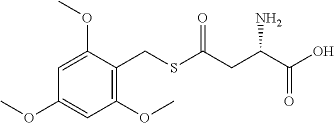 [SMILES: COc1cc(OC)c(CSC(=O)C[C@H](N)C(=O)O)c(OC)c1]

or a salt thereof.
2055. A compound, wherein the compound is

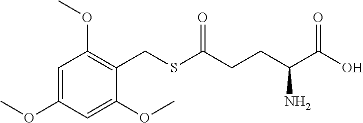 [SMILES: COc1cc(OC)c(CSC(=O)CC[C@H](N)C(=O)O)c(OC)c1]

or a salt thereof.
2056. The compound of any one of the preceding Embodiments, wherein the compound has a purity of at least about 90%, 91%, 92%, 93%, 94%, 95%, 96%, 97%, 98%, or 99%.
2057. A compound, comprising a residue of any one of the preceding Embodiments.
2058. A compound, comprising a residue of Table A-IV.
2059. A compound, comprising a residue having the structure of

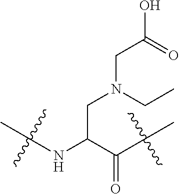 [SMILES: *NC(CN(CC)CC(=O)O)C(*)=O]

or a salt form thereof.
2060. A compound, comprising a residue having the structure of

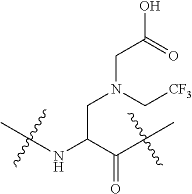 [SMILES: *NC(CN(CC(=O)O)CC(F)(F)F)C(*)=O]

or a salt form
2061. A compound, comprising a residue having the structure of

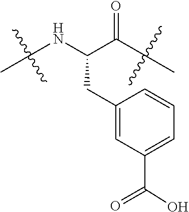 [SMILES: *N[C@@H](Cc1cccc(C(=O)O)c1)C(*)=O]

H or a salt form thereof.
2062. A compound, comprising a residue having the structure of

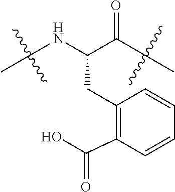 [SMILES: *N[C@@H](Cc1ccccc1C(=O)O)C(*)=O]

or a salt form thereof.
2063. A compound, comprising a residue having the structure of

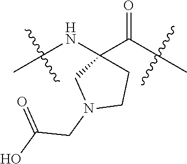 [SMILES: *N[C@@]1(C(*)=O)CCN(CC(=O)O)C1]

or a salt form thereof.
2064. A compound, comprising a residue having the structure of

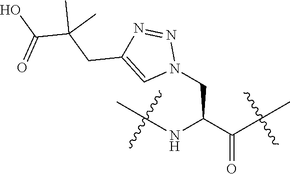 [SMILES: *N[C@@H](Cn1cc(CC(C)(C)C(=O)O)nn1)C(*)=O]

or a salt form thereof.
2065. A compound, comprising a residue having the structure of

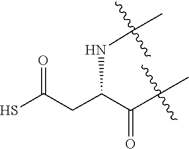 [SMILES: *N[C@@H](CC(=O)S)C(*)=O]

or a salt form thereof.
2066. A compound, comprising a residue having the structure of

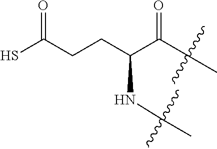 [SMILES: *N[C@@H](CCC(=O)S)C(*)=O]

or a salt form thereof.
2067. A compound, comprising a residue having the structure of

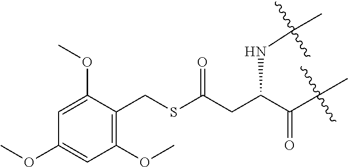 [SMILES: *N[C@@H](CC(=O)SCc1c(OC)cc(OC)cc1OC)C(*)=O]

or a salt form thereof.
2068. A compound, comprising a residue having the structure of

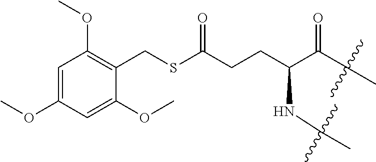 [SMILES: *N[C@@H](CCC(=O)SCc1c(OC)cc(OC)cc1OC)C(*)=O]

or a salt form thereof 2069. The compound of any one of Embodiments 2057-2068, wherein the compound is or comprise a peptide.
2070. The compound of any one of Embodiments 2057-2068, wherein the compound is an agent of any one of the preceding Embodiments.
2071. The compound of any one of Embodiments 2057-2068, wherein the compound is or comprise a stapled peptide.
2072. A method for preparing a compound of any one of Embodiments 2057-2071, comprising utilization of a compound of any one of the Embodiments 1901-2056.
2073. An agent, which agent comprises a residue of an amino acid of any one of the preceding Embodiments.
2074. The agent of any one of Embodiments 1-1900, wherein the agent comprises a residue of an amino acid of any one of the preceding Embodiments.
2075. The agent of any one of the preceding Embodiments, wherein each olefin double bond in a staple is independently and optionally converted into a single bond.
2076. The agent of any one of the preceding Embodiments, wherein each olefin double bond in a staple is converted into a single bond.
2077. The agent of any one of the preceding Embodiments, wherein each olefin double bond is converted into a single bond.
2078. The agent of any one of the preceding Embodiments, wherein each olefin double bond is independently and optionally converted into —CHR′—CHR′—, wherein each R is independently —H, —R, —OR, —OH, —N(R)₂, or —SR.
2079. The agent of any one of the preceding Embodiments, wherein each olefin double bond is converted into —CHR′—CHR′—, wherein each R is independently —H, —R, —OR, —OH, —N(R)₂, or —SR.
2080. The agent of any one of the preceding Embodiments, wherein each olefin double bond is independently and optionally converted into optionally substituted —CH₂—CH₂—.
2081. The agent of any one of the preceding Embodiments, wherein each olefin double bond is converted into —CH₂—CH₂—.
2082. The agent of any one of the preceding Embodiments, having a diastereopurity of about 80%, 85%, 90%, 91%, 92%, 93%, 94%, 95%, 96%, 97%, 98%, 99% or more.
2083. The agent of any one of the preceding Embodiments, having a diastereopurity of about 90% or more.
2084. The agent of any one of the preceding Embodiments, having a diastereopurity of about 95% or more.
2085. The agent of any one of the preceding Embodiments, having a diastereopurity of about 98% or more.
2086. The agent of any one of the preceding Embodiments, having a purity of about 80%, 85%, 90%, 91%, 92%, 93%, 94%, 95%, 96%, 97%, 98%, 99% or more.
2087. The agent of any one of the preceding Embodiments, having a purity of about 90% or more.
2088. The agent of any one of the preceding Embodiments, having a purity of about 95% or more.
2089. The agent of any one of the preceding Embodiments, having a purity of about 98% or more.
2090. A composition comprising an agent of any one of the preceding Embodiments or a salt thereof.
2091. A pharmaceutical composition, comprising or delivering an agent or amino acid of any one of the preceding Embodiments, and a pharmaceutically acceptable carrier.
2092. A composition selected from Table E2.
2093. A pharmaceutical composition, comprising or delivering one or more or all peptide agents in a composition selected from Table E2 and a pharmaceutically acceptable carrier.
2094. A composition selected from Table E3.
2095. A pharmaceutical composition, comprising or delivering one or more or all peptide agents in a composition selected from Table E3 and a pharmaceutically acceptable carrier.
2096. The composition of any one of the preceding Embodiments, comprising an agent comprising one or more staples each independently comprises one or more olefin double bond.
2097. The composition of any one of the preceding Embodiments, wherein the ratio of the two stereoisomers of an olefin double bond in a staple is about 2:1, 3:1, 4:1, 5:1, 6:1, 7:1, 8:1, 9:1, 10:1, 15:1, 20:1, 30:1, 40:1, 50:1 or more.
2098. The composition of Embodiment 2097, wherein the ratio is about 5:1 or more.
2099. The composition of Embodiment 2097, wherein the ratio is about 10:1 or more.
2100. The composition of Embodiment 2097, wherein the ratio is about 20:1 or more.
2101. The composition of Embodiment 2097, wherein the ratio is about 50:1 or more.
2102. The composition of any one of the preceding Embodiments, wherein each ratios of the two stereoisomers of each olefin double bond in each staple are independently about 2:1, 3:1, 4:1, 5:1, 6:1, 7:1, 8:1, 9:1, 10:1, 15:1, 20:1, 30:1, 40:1, 50:1 or more.
2103. The composition of Embodiment 2102, wherein each ratio is independently about 5:1 or more.
2104. The composition of Embodiment 2102, wherein each ratio is independently about 10:1 or more.
2105. The composition of Embodiment 2102, wherein each ratio is independently about 20:1 or more.
2106. The composition of Embodiment 2102, wherein each ratio is independently about 50:1 or more.
2107. The composition of any one of the preceding Embodiments, wherein a selectivity is favoring an E configuration.
2108. The composition of any one of the preceding Embodiments, wherein a selectivity is favoring a Z configuration.
2109. A method for preparing an agent or composition of any one of the preceding Embodiments, comprising incorporating a residue of an amino acid of any one of the preceding Embodiments.
2110. The method of Embodiment 2109, comprising preparing a compound comprising one or more amino acid residues comprising terminal olefins, wherein one or more or all of such amino acid residues are not stapled.
2111. A method, comprising
   a) preparing a first compound comprising two moieties each of which independently comprises an olefin double bond;
   b) providing a second compound by stapling the two moieties by olefin metathesis of an olefin double bond of one moiety with an olefin double bond of the other to form a first-formed staple;
   c) add one or more additional moieties to the second compound to provide a third compound which comprising two moieties each of which independently comprises an olefin double bond; and
   d) providing a fourth compound by stapling the two moieties in the third compound by olefin metathesis of an olefin double bond of one moiety with an olefin double bond of the other to form a second-formed staple.
2112. The method of Embodiment 2111, wherein each moiety is independently an amino acid residue comprising a terminal olefin of any one of the preceding Embodiments.
2113. The method of any one of the preceding Embodiments, wherein there are two olefin double bonds in one moiety of the first compound.
2114. The method of any one of the preceding Embodiments, wherein a moiety in a first compound is an amino acid residue comprising two olefin double bond.
2115. The method of any one of the preceding Embodiments, wherein one moiety in a first compound is B5.
2116. The method of any one of Embodiments, wherein the two moieties of the first compound is independently X⁴ and X¹¹.
2117. The method of any one of the preceding Embodiments, wherein a first-formed staple is a (i, i+7) staple.
2118. The method of any one of the preceding Embodiments, wherein the first compound comprises —X⁴X⁵X⁶X⁷X⁸X⁹X¹⁰X¹¹—.
2119. The method of any one of the preceding Embodiments, wherein the first compound comprises —X⁴X⁵X⁶X⁷X⁸X⁹X¹⁰X¹¹X¹²X¹³X¹⁴—.
2120. The method of any one of the preceding Embodiments, wherein the first compound comprises a staple.
2121. The method of Embodiment 2120, wherein the staple is a (i, i+4) staple.
2122. The method of Embodiment 2120, wherein the staple is between X¹⁰ and X¹⁴.
2123. The method any one of the preceding Embodiments, wherein an olefin double bond in the third compound is present in the first compound.
2124. The method of any one of the preceding Embodiments, wherein one and only one amino acid residue comprises an olefin double bond is added to the second compound.
2125. The method of any one of the preceding Embodiments, wherein the third compound is or comprises —X¹X²X³X⁴X⁵X⁶X⁷X⁸X⁹X¹⁰X¹¹—.
2126. The method of any one of the preceding Embodiments, wherein the third compound is or comprises —X¹X²X³X⁴X⁵X⁶X⁷X⁸X⁹X¹⁰X¹¹X¹²X¹³X¹⁴—.
2127. The method of any one of the preceding Embodiments, wherein the first- and second-formed staples are bonded to the same amino acid residue.
2128. The method of any one of the preceding Embodiments, wherein the first- and second-formed staples are bonded to the same atom.
2129. The method of any one of the preceding Embodiments, wherein the second-formed staple is a (i, i+2), (i, i+3) or (i, i+4) staple.
2130. The method of any one of the preceding Embodiments, wherein the two moieties in the third compound is independently X¹ and X⁴.
2131. The method of any one of the preceding Embodiments, wherein the first-formed staple is formed with E selectivity.
2132. The method of any one of the preceding Embodiments, wherein the second-formed staple is formed with Z selectivity.
2133. The method of any one of Embodiments 2111-2132, wherein an agent of any one of the preceding Embodiments is prepared.
2134. The method of any one of the preceding Embodiments, comprising preparing a compound having an amino acid sequence of Table E2 or Table E3 but one or more or all amino acid residues comprising terminal olefins are not stapled.
2135. The method of any one of Embodiments 2109-2134, comprising stapling two or more amino acid residues each independently comprising one or more olefins to form one or more staples each independently comprising a carbon-carbon double bond.
2136. The method of Embodiment 2135, wherein the stapling is performed via olefin metathesis of terminal olefins.
2137. The method of any one of Embodiments 2109-2136, wherein a double bond in a staple is formed with about 1.1:1, 1.2:1, 1.5:1, 2:1, 3:1, 4:1, 5:1, 6:1, 7:1, 8:1, 9:1, 10:1, 15:1, 20:1 or more stereoselectivity.
2138. The method of Embodiment 2137, wherein the selectivity is about 1.5:1 or more.
2139. The method of Embodiment 2137, wherein the selectivity is about 2:1 or more.
2140. The method of Embodiment 2137, wherein the selectivity is about 3:1 or more.
2141. The method of Embodiment 2137, wherein the selectivity is about 4:1 or more.
2142. The method of Embodiment 2137, wherein the selectivity is about 9:1 or more.
2143. The method of Embodiment 2137, wherein the selectivity is about 10:1 or more
2144. The method of Embodiment 2137-2143, wherein the staple is a first-formed staple.
2145. The method of any one of Embodiments 2109-2144, wherein each double bond in each staple is independently formed with about 1.1:1, 1.2:1, 1.5:1, 2:1, 3:1, 4:1, 5:1, 6:1, 7:1, 8:1, 9:1, 10:1, 15:1, 20:1 or more stereoselectivity.
2146. The method of Embodiment 2145, wherein the selectivity is independently about 1.5:1 or more.
2147. The method of Embodiment 2145, wherein the selectivity is independently about 2:1 or more.
2148. The method of Embodiment 2145, wherein the selectivity is independently about 3:1 or more.
2149. The method of Embodiment 2145, wherein the selectivity is independently about 4:1 or more.
2150. The method of Embodiment 2145, wherein the selectivity is independently about 9:1 or more.
2151. The method of Embodiment 2145, wherein the selectivity is independently about 10:1 or more.
2152. The method of any one of Embodiments 2137-2151, wherein a selectivity is favoring an E isomer.
2153. The method of any one of Embodiments 2137-2151, wherein a selectivity is favoring a Z isomer.
2154. The method of any one of Embodiments 2109-2153, comprising purifying a composition to enrich one or more E/Z stereoisomers.
2155. The method of Embodiment 2154, wherein one configuration of an olefin double bond in a staple is enriched.
2156. The method of Embodiment 2155, wherein the ratio after enrichment is about 2:1, 3:1, 4:1, 5:1, 6:1, 7:1, 8:1, 9:1, 10:1, 15:1, 20:1, 30:1, 40:1, 50:1 or more.
2157. The method of Embodiment 2156, wherein the ratio is about 5:1 or more.
2158. The method of Embodiment 2156, wherein the ratio is about 10:1 or more.
2159. The method of Embodiment 2156, wherein the ratio is about 20:1 or more.
2160. The method of Embodiment 2156, wherein the ratio is about 50:1 or more.
2161. The method of Embodiment 2154, wherein configuration of each olefin double bond in each staple is independently enriched.
2162. The method of Embodiment 2161, wherein the ratio for each olefin double bond after enrichment is independently about 2:1, 3:1, 4:1, 5:1, 6:1, 7:1, 8:1, 9:1, 10:1, 15:1, 20:1, 30:1, 40:1, 50:1 or more.
2163. The method of Embodiment 2162, wherein each ratio is independently about 5:1 or more.
2164. The method of Embodiment 2162, wherein each ratio is independently about 10:1 or more.
2165. The method of Embodiment 2162, wherein each ratio is independently about 20:1 or more.
2166. The method of Embodiment 2162, wherein each ratio is independently about 50:1 or more.
2167. The method of any one of Embodiments 2154-2166, wherein a selectivity is favoring an E configuration.
2168. The method of any one of Embodiments 2154-2167, wherein a selectivity is favoring a Z configuration.
2169. The method of any one of the preceding Embodiments, wherein a chiral center is formed with about or at least about 60%, 65%, 70%, 75%. 80%, 85%, 90%, 95%. 98% or 99% stereoselectivity.
2170. The method of any one of the preceding Embodiments, wherein a chiral center is formed with about or at least about 80% or more stereoselectivity.
2171. The method of any one of the preceding Embodiments, wherein a chiral center is formed with about or at least about 85% or more stereoselectivity.
2172. The method of any one of the preceding Embodiments, wherein a chiral center is formed with about or at least about 90% or more stereoselectivity.
2173. The method of any one of the preceding Embodiments, wherein a chiral center is formed with about or at least about 95% or more stereoselectivity.
2174. The method of any one of Embodiments 2169-2173, wherein the chiral center is bonded to two staples.
2175. The method of any one of Embodiments 2109-2174, comprising modifying a double bond in a staple.
2176. The method of any one of Embodiments 2109-2174, comprising hydrogenating a double bond in a staple.
2177. The method of any one of Embodiments 2109-2174, comprising hydrogenating each carbon-carbon double bond in each staple.
2178. The method of any one of the preceding Embodiments, comprising purifying a composition by chromatography and providing one or more compositions based on peak(s) observed during purification.
2179. The method of any one of the preceding Embodiments, comprising purifying a composition by liquid chromatography and providing one or more compositions based on peak(s) observed during purification.
2180. The method of any one of Embodiments 2178-2179, wherein the chromatography purification utilizes the same or similar conditions with respect to separation of peaks with the correct mass as those described for Table E2 or Table E3.
2181. The method of any one of Embodiments 2178-2180, wherein the chromatography purification utilizes the same or similar conditions with respect to elution order of peaks with the correct mass as those described for Table E2 or Table E3.
2182. The method of any one of the preceding Embodiments, comprising collecting the first peak with the correct mass as a product composition.
2183. The method of any one of the preceding Embodiments, comprising collecting the second peak with the correct mass as a product composition.
2184. The method of any one of the preceding Embodiments, comprising collecting the third peak with the correct mass as a product composition.
2185. The method of any one of the preceding Embodiments, comprising collecting the fourth peak with the correct mass as a product composition.
2186. The method of any one of the preceding Embodiments, comprising collecting each peak with the correct mass as a product composition.
2187. The method of any one of the preceding Embodiments, wherein the peak area of a product composition is about 5%, 10%, 20%, 30%, 40%, 50%, 60%, 70%,80%, 90% or more of the total peak area of all peak(s) with the correct mass.
2188. The method of Embodiment 2187, where the percentage is 10% or more.
2189. The method of Embodiment 2187, where the percentage is 20% or more.
2190. The method of Embodiment 2187, where the percentage is 50% or more.
2191. The method of Embodiment 2187, where the percentage is 60% or more.
2192. The method of any one of the preceding Embodiments, wherein the peak area of each product composition is about 5%, 10%, 20%, 30%, 40%, 50%, 60%, 70%,80%, 90% or more of the total peak area of all peak(s) with the correct mass.
2193. The method of Embodiment 2192, wherein each percentage is independently 10% or more.
2194. The method of Embodiment 2192, wherein each percentage is independently 20% or more.
2195. The method of Embodiment 2192, wherein each percentage is independently 50% or more.
2196. The method of Embodiment 2192, wherein each percentage is independently 60% or more.
2197. The method of any one of Embodiments 2187-2196, wherein the peak is from MS detection.
2198. The method of any one of Embodiments 2187-2197, wherein the peak is from UV detection.
2199. The method of any one of Embodiments 2187-2197, wherein the peak is from UV detection at 220 nm.
2200. A composition produced from a method of any one of the preceding Embodiments.
2201. A pharmaceutical composition comprising or delivering a composition of Embodiment 2200 and a pharmaceutically acceptable carrier.
2202. A method for modulating beta-catenin interaction with a partner in a system, comprising contacting beta-catenin with an agent or composition of any one of the preceding Embodiments.
2203. A method for modulating beta-catenin interaction with a partner in a system, comprising administering or delivering to the system an agent or composition of any one of the preceding Embodiments.
2204. The method of nay one of Embodiments 2202-2203, wherein the partner is TCF7, LEF1, TCF7L1, TCF7L2, Axin1, Axin2, or APC.
2205. A method for modulating a TCF-beta-catenin interaction in a system, comprising contacting beta-catenin with an agent or composition of any one of the preceding Embodiments.
2206. A method for modulating a TCF-beta-catenin interaction in a system, comprising administering or delivering to the system an agent or composition of any one of the preceding Embodiments.
2207. A method for inhibiting beta-catenin dependent cell proliferation, comprising administering or delivering to the system an agent or composition of any one of the preceding Embodiments.
2208. A method for modulating WNT/beta-catenin pathway in a system, comprising administering or delivering to the system an agent or composition of any one of the preceding Embodiments, wherein expression of a nucleic acid is modulated.
2209. A method, comprising administering or delivering to the system an agent or composition of any one of the preceding Embodiments, wherein level of a transcript of a nucleic acid and/or a product thereof is modulated.
2210. A method, comprising administering or delivering to the system an agent or composition of any one of the preceding Embodiments, wherein expression of a nucleic acid is modulated.
2211. The method of any one of Embodiments 2208-2210, wherein a nucleic acid is or comprises a gene.
2212. The method of any one of Embodiments 2208-2211, wherein a nucleic acid is selected from gene set BCAT_GDS748-UP or Table GS1.
2213. The method of any one of Embodiments 2208-2212, wherein a nucleic acid is selected from gene set BCAT.100-UP.V1-UP or Table GS2.
2214. The method of any one of Embodiments 2208-2213, wherein a nucleic acid is selected from gene set HALLMARK_WNT_BETA_CATENIN_SIGNALING or Table GS3.
2215. The method of any one of Embodiments 2208-2214, wherein a nucleic acid is selected from gene set RASHI_RESPONSE_TO_IONIZING_RADIATION_1 or Table GS4.
2216. The method of any one of Embodiments 2208-2215, wherein a nucleic acid is selected from gene set REACTOME_RRNA_PROCESSING or Table GS5.
2217. The method of any one of Embodiments 2208-2216, wherein a nucleic acid is selected from gene set HALLMARK_MYC_TARGETS_V1 or Table GS6.
2218. The method of any one of Embodiments 2208-2217, wherein a nucleic acid is selected from gene set HALLMARK_MYC_TARGETS_V2 or Table GS7.
2219. The method of any one of Embodiments 2208-2218, wherein a nucleic acid is selected from gene set HALLMARK_OXIDATIVE_PHOSPHORYLATION or Table GS8.
2220. The method of any one of Embodiments 2208-2219, wherein a nucleic acid is selected from gene set HALLMARK_E2F_TARGETS or Table GS9.
2221. The method of any one of Embodiments 2208-2220, wherein a nucleic acid is selected from gene set HALLMARK_TNFA_SIGNALING_VIA_NFKB or Table GS10.
2222. The method of any one of Embodiments 2208-2221, wherein a nucleic acid is SP5.
2223. The method of any one of Embodiments 2208-2222, wherein a nucleic acid is CCND2.
2224. The method of any one of Embodiments 2208-2223, wherein a nucleic acid is WNT5B.
2225. The method of any one of Embodiments 2208-2224, wherein a nucleic acid is AXIN2.
2226. The method of any one of Embodiments 2208-2225, wherein a nucleic acid is NKD1.
2227. The method of any one of Embodiments 2208-2226, wherein a nucleic acid is WNT6.
2228. The method of any one of Embodiments 2208-2227, wherein a nucleic acid is DKK1.
2229. The method of any one of Embodiments 2208-2228, wherein a nucleic acid is DKK4.
2230. The method of any one of Embodiments 2208-2229, wherein expression of the nucleic acid is reduced.
2231. The method of any one of Embodiments 2208-2230, wherein BCAT_GDS748 UP is negatively enriched.
2232. The method of any one of Embodiments 2208-2231, wherein BCAT.100-UP.V1-UP is negatively enriched.
2233. The method of any one of Embodiments 2208-2232, wherein HALLMARK_WNT_BETA_CATENIN_SIGNALING is negatively enriched.
2234. The method of any one of Embodiments 2208-2233, wherein RASHI_RESPONSE_TO_IONIZING_RADIATION_1 is negatively enriched.
2235. The method of any one of Embodiments 2208-2234, wherein REACTOME_RRNA_PROCESSING is negatively enriched.
2236. The method of any one of Embodiments 2208-2235, wherein HALLMARK_MYC_TARGETS_V1 is negatively enriched.
2237. The method of any one of Embodiments 2208-2236, wherein HALLMARK_MYC_TARGETS_V2 is negatively enriched.
2238. The method of any one of Embodiments 2208-2237, wherein HALLMARK_OXIDATIVE_PHOSPHORYLATION is negatively enriched.
2239. The method of any one of Embodiments 2208-2238, wherein HALLMARK_E2F_TARGETS is negatively enriched.
2240. The method of any one of Embodiments 2208-2239, wherein HALLMARK_TNFA_SIGNALING_VIA_NFKB is negatively enriched.
2241. The method of any one of Embodiments 2208-2240, wherein expression of the nucleic acid is reduced.
2242. The method of any one of Embodiments 2208-2241, wherein level of the transcript and/or a product thereof is reduced.
2243. The method of any one of Embodiments 2208-2242, wherein expression of a nucleic acid is increased.
2244. The method of any one of Embodiments 2208-2243, wherein level of a transcript of a nucleic acid or a product thereof is increased.
2245. The method of any one of Embodiments 2243-2244, wherein the nucleic acid is or comprises CXCL12 gene 2246. The method of any one of Embodiments 2208-2245, wherein one or more gene sets are independently positively enriched.
2247. The method of any one of Embodiments 2202-2246, wherein a system is an in vitro system.
2248. The method of any one of Embodiments 2202-2246, wherein a system is an in vivo system.
2249. The method of any one of Embodiments 2202-2246, wherein a system is or comprises a sample.
2250. The method of any one of Embodiments 2202-2249, wherein a system is or comprises a cell, tissue or organ.
2251. The method of any one of Embodiments 2202-2250, wherein a system is or comprises cancer cells.
2252. The method of any one of Embodiments 2202-2251, wherein a system is or comprises colorectal cancer cells.
2253. The method of any one of Embodiments 2202-2253, wherein a system is or comprises COLO320DM cells.
2254. The method of any one of Embodiments 2202-2253, wherein a system is or comprises a tumor.
2255. The method of any one of Embodiments 2202-2254, wherein a system is a subject.
2256. A method for treating or preventing a condition, disorder or disease associated with beta-catenin in a subject, comprising administering or delivering to the subject an effective amount of an agent or composition of any one of the preceding Embodiments.
2257. A method for treating cancer in a subject, comprising administering or delivering to the subject an effective amount of an agent or composition of any one of the preceding Embodiments.
2258. A method for treating or preventing a condition, disorder or disease associated with beta-catenin interaction with a partner in a subject, comprising administering or delivering to the subject an effective amount of an agent or composition of any one of the preceding Embodiments.
2259. The method of Embodiment 2258, wherein the partner is TCF7, LEF1, TCF7L1, TCF7L2, Axin1, Axin2, or APC.
2260. A method for treating or preventing a condition, disorder or disease associated with TCF-beta-catenin interaction in a subject, comprising administering or delivering to the subject an effective amount of an agent or composition of any one of the preceding Embodiments.
2261. The method of any one of the preceding Embodiments, wherein the condition, disorder or disease is melanoma.
2262. The method of any one of the preceding Embodiments, comprising administering or deliver to a subject a second therapeutic agent.
2263. The method of any one of the preceding Embodiments, comprising administering or deliver to a subject a second therapy.
2264. The method of Embodiment 2262 or 2263, wherein a second therapeutic agent or therapy is administered prior to an agent of any one of the preceding Embodiments.
2265. The method of Embodiment 2262 or 2263, wherein a second therapeutic agent or therapy is administered about or no more than about 1, 2, 3, 4, 5, 6, or 7 days, or 1, 2, 3, or weeks, or 1, 2, 3, 4, 5, or 6 months, prior to an agent of any one of the preceding Embodiments.
2266. The method of Embodiment 2262 or 2263, wherein a second therapeutic agent or therapy is administered concurrently with an agent of any one of the preceding Embodiments.
2267. The method of Embodiment 2262 or 2263, wherein a second therapeutic agent or therapy is administered subsequently to an agent of any one of the preceding Embodiments.
2268. The method of Embodiment 2262 or 2263, wherein a second therapeutic agent or therapy is administered about or no more than about 1, 2, 3, 4, 5, 6, or 7 days, or 1, 2, 3, or weeks, or 1, 2, 3, 4, 5, or 6 months, subsequently to an agent of any one of the preceding Embodiments.
2269. The method of any one of the preceding Embodiments, wherein a subject is exposed to a second therapeutic agent or therapy and an agent of any one of the preceding Embodiments.
2270. The method of any one of the preceding Embodiments, wherein a subject is exposed to a therapeutic effect of a second therapeutic agent or therapy and a therapeutic effect of an agent of any one of the preceding Embodiments.
2271. The method of any one of the preceding Embodiments, wherein a second therapeutic agent is or comprises a chemotherapy agent.
2272. The method of any one of the preceding Embodiments, wherein a second therapeutic agent is or comprises a hormone therapy agent.
2273. The method of any one of the preceding Embodiments, wherein a second therapeutic agent is or comprises an immunotherapy agent.
2274. The method of any one of the preceding Embodiments, wherein a second therapeutic agent is or comprises a checkpoint inhibitor.
2275. The method of any one of the preceding Embodiments, wherein a second therapeutic agent is or comprises an antibody.
2276. The method of any one of the preceding Embodiments, wherein a second therapeutic agent is or comprises a CTLA-4, PD-1 or PD-L1 inhibitor.
2277. The method of any one of the preceding Embodiments, wherein a second therapeutic agent is or comprises a cell.
2278. The method of any one of the preceding Embodiments, wherein the second therapeutic agent reduces one or more side effects of an agent or composition of any one of the preceding Embodiments.
2279. The method of any one of the preceding Embodiments, wherein the agent or composition reduces one or more side effects of a second therapeutic agent.
2280. The method of any one of the preceding Embodiments, wherein a second therapy is or comprises surgery.
2281. The method of any one of the preceding Embodiments, wherein a second therapy is or comprises chemotherapy.
2282. The method of any one of the preceding Embodiments, wherein a second therapy is or comprises radiotherapy.
2283. The method of any one of the preceding Embodiments, wherein a second therapy is or comprises hormone therapy.
2284. The method of any one of the preceding Embodiments, wherein a second therapy is or comprises stem cell or bone marrow transplant.
2285. The method of any one of the preceding Embodiments, wherein a second therapy is or comprises immunotherapy.
2286. The method of any one of the preceding Embodiments, wherein a second therapy is or comprises T-cell therapy.
2287. The method of any one of the preceding Embodiments, wherein a second therapy is or comprises CAR T-cell therapy.
2288. The method of any one of the preceding Embodiments, wherein a second therapy is or comprises administering to the subject a population of immune cells.
2289. The method of any one of the preceding Embodiments, wherein the agent or composition reduces one or more side effects of a second therapy.
2290. The method of any one of the preceding Embodiments, wherein unit dose of a second therapy or therapeutic agent is reduced compared to when it is administered alone.
2291. The method of any one of the preceding Embodiments, wherein total dose of a second therapy or therapeutic agent is reduced compared to when it is administered alone.
2292. The method of any one of the preceding Embodiments, wherein unit dose of an agent or composition of any one of the preceding Embodiments is reduced compared to when it is administered alone.
2293. The method of any one of the preceding Embodiments, wherein total dose of an agent or composition of any one of the preceding Embodiments is reduced compared to when it is administered alone.
2294. The method of any one of the preceding Embodiments, wherein the combination therapy provides higher efficacy than when an agent or composition is administered or delivered alone.
2295. The method of any one of the preceding Embodiments, wherein the combination therapy provides higher efficacy than when a second therapeutic agent or therapy is administered or delivered alone.
2296. The method of any one of the preceding Embodiments, comprising assessing expression of a nucleic acid.
2297. The method of any one of the preceding Embodiments, wherein expression of a nucleic acid is modulated.
2298. The method of any one of the preceding Embodiments, comprising assessing level of a transcript of a nucleic acid and/or a product thereof.
2299. The method of any one of the preceding Embodiments, wherein level of a transcript of a nucleic acid and/or a product thereof is modulated.
2300. The method of any one of the preceding Embodiments, comprising collecting a sample from a subject, and assessing expression of a nucleic acid in the sample.
2301. The method of any one of the preceding Embodiments, comprising collecting a sample from a subject, wherein expression of a nucleic acid in the sample is modulated.
2302. The method of any one of the preceding Embodiments, comprising collecting a sample from a system, and assessing expression of a nucleic acid in the sample.
2303. The method of any one of the preceding Embodiments, comprising collecting a sample from a system, wherein expression of a nucleic acid in the sample is modulated.
2304. The method of any one of the preceding Embodiments, comprising collecting a sample from a subject, and assessing level of a transcript of a nucleic acid and/or a product thereof in the sample.
2305. The method of any one of the preceding Embodiments, comprising collecting a sample from a subject, and level of a transcript of a nucleic acid and/or a product thereof in the sample is modulated.
2306. The method of any one of the preceding Embodiments, comprising collecting a sample from a system, and assessing level of a transcript of a nucleic acid and/or a product thereof in the sample.
2307. The method of any one of the preceding Embodiments, comprising collecting a sample from a system, and level of a transcript of a nucleic acid and/or a product thereof in the sample is modulated.
2308. The method of any one of Embodiments 2296-2307, wherein a sample is or comprises a cell, tissue or organ.
2309. The method of any one of Embodiments 2296-2308, wherein a sample is or comprises cancer cells.
2310. The method of any one of Embodiments 2296-2309, wherein a sample is or comprises colorectal cancer cells.
2311. The method of any one of Embodiments 2296-2310, wherein a sample is or comprises COLO320DM cells.
2312. The method of any one of Embodiments 2296-2311, wherein a sample comprises cells from a tumor.
2313. The method of any one of Embodiments 2296-2312, wherein a sample comprises tissues from a tumor.
2314. The method of any one of Embodiments 2296-2313, wherein a sample is or comprises a tumor.
2315. The method of any one of Embodiments 2296-2311, wherein a sample is from a tumor.
2316. The method of any one of Embodiments 2296-2315, wherein a sample is from a biopsy.
2317. The method of any one of Embodiments 2296-2316, wherein a sample is collected after one or more administrations or deliveries.
2318. The method of any one of Embodiments 2296-2317, wherein an assessment is conducted after one or more administrations or deliveries.
2319. The method of any one of Embodiments 2296-2318, wherein a nucleic acid is or comprises a gene.
2320. The method of any one of Embodiments 2296-2319, wherein a nucleic acid is selected from gene set BCAT_GDS748-UP or Table GS1.
2321. The method of any one of Embodiments 2296-2320, wherein a nucleic acid is selected from gene set BCAT.100-UP.V1-UP or Table GS2.
2322. The method of any one of Embodiments 2296-2321, wherein a nucleic acid is selected from gene set HALLMARK_WNT_BETA_CATENIN_SIGNALING or Table GS3.
2323. The method of any one of Embodiments 2296-2322, wherein a nucleic acid is selected from gene set RASHI_RESPONSE_TO_IONIZING_RADIATION_1 or Table GS4.
2324. The method of any one of Embodiments 2296-2323, wherein a nucleic acid is selected from gene set REACTOME_RRNA_PROCESSING or Table GS5.
2325. The method of any one of Embodiments 2296-2324, wherein a nucleic acid is selected from gene set HALLMARK_MYC_TARGETS_V1 or Table GS6.
2326. The method of any one of Embodiments 2296-2325, wherein a nucleic acid is selected from gene set HALLMARK_MYC_TARGETS_V2 or Table GS7.
2327. The method of any one of Embodiments 2296-2326, wherein a nucleic acid is selected from gene set HALLMARK_OXIDATIVE_PHOSPHORYLATION or Table GS8.
2328. The method of any one of Embodiments 2296-2327, wherein a nucleic acid is selected from gene set HALLMARK_E2F_TARGETS or Table GS9.
2329. The method of any one of Embodiments 2296-2328, wherein a nucleic acid is selected from gene set HALLMARK_TNFA_SIGNALING_VIA_NFKB or Table GS10.
2330. The method of any one of Embodiments 2296-2329, wherein a nucleic acid is CCND2.
2331. The method of any one of Embodiments 2296-2330, wherein a nucleic acid is WNT5B.
2332. The method of any one of Embodiments 2296-2331, wherein a nucleic acid is AXIN2.
2333. The method of any one of Embodiments 2296-2332, wherein a nucleic acid is NKD1.
2334. The method of any one of Embodiments 2296-2333, wherein a nucleic acid is WNT6.
2335. The method of any one of Embodiments 2296-2334, wherein a nucleic acid is DKK1.
2336. The method of any one of Embodiments 2296-2335, wherein a nucleic acid is DKK4.
2337. The method of any one of Embodiments 2296-2336, wherein expression of the nucleic acid is reduced.
2338. The method of any one of Embodiments 2296-2337, wherein BCAT_GDS748 UP is negatively enriched.
2339. The method of any one of Embodiments 2296-2338, wherein BCAT.100-UP.V1-UP is negatively enriched.
2340. The method of any one of Embodiments 2296-2339, wherein HALLMARK_WNT_BETA_CATENIN_SIGNALING is negatively enriched.
2341. The method of any one of Embodiments 2296-2340, wherein RASHI_RESPONSE_TO_IONIZING_RADIATION_1 is negatively enriched.
2342. The method of any one of Embodiments 2296-2341, wherein REACTOME_RRNA_PROCESSING is negatively enriched.
2343. The method of any one of Embodiments 2296-2342, wherein HALLMARK_MYC_TARGETS_V1 is negatively enriched.
2344. The method of any one of Embodiments 2296-2343, wherein HALLMARK_MYC_TARGETS_V2 is negatively enriched.
2345. The method of any one of Embodiments 2296-2344, wherein HALLMARK_OXIDATIVE_PHOSPHORYLATION is negatively enriched.
2346. The method of any one of Embodiments 2296-2345, wherein HALLMARK_E2F_TARGETS is negatively enriched.
2347. The method of any one of Embodiments 2296-2346, wherein HALLMARK_TNFA_SIGNALING_VIA_NFKB is negatively enriched.
2348. The method of any one of Embodiments 2296-2347, wherein expression of the nucleic acid is reduced.
2349. The method of any one of Embodiments 2296-2348, wherein level of the transcript and/or a product thereof is reduced.
2350. The method of any one of Embodiments 2296-2349, wherein expression of a nucleic acid is increased.
2351. The method of any one of Embodiments 2296-2350, wherein level of a transcript of a nucleic acid or a product thereof is increased.
2352. The method of any one of Embodiments 2350-2351, wherein the nucleic acid is or comprises CXCL12 gene
2353. The method of any one of Embodiments 2296-2352, wherein one or more gene sets are independently positively enriched.
2354. The method of any one of Embodiments 2296-2353, wherein administration or delivery continues for one or more times after the assessment.
2355. The method of any one of Embodiments 2296-2354, comprising evaluating an assessment and continue the administration or delivery.
2356. The method of any one of Embodiments 2296-2355, wherein administration or deliver is adjusted after an assessment.
2357. The method of any one of Embodiments 2296-2356, comprising evaluating an assessment and adjusting the administration or delivery.
2358. The method of any one of Embodiments 2296-2319, wherein administration or deliver is discontinued after an assessment.
2359. The method of any one of Embodiments 2296-2318 and 2358, comprising evaluating an assessment and discontinuing the administration or delivery.
2360. The method of any one of Embodiments 2356-2359, wherein expression of SP5 remains about the same or is increased.
2361. The method of any one of Embodiments 2356-2360, wherein expression of CCND2 remains about the same or is increased.
2362. The method of any one of Embodiments 2356-2361, wherein expression of WNT5B remains about the same or is increased.
2363. The method of any one of Embodiments 2356-2362, wherein expression of AXIN2 remains about the same or is increased.
2364. The method of any one of Embodiments 2356-2363, wherein expression of NKD1 remains about the same or is increased.
2365. The method of any one of Embodiments 2356-2364, wherein expression of WNT6 remains about the same or is increased.
2366. The method of any one of Embodiments 2356-2365, wherein expression of DKK1 remains about the same or is increased.
2367. The method of any one of Embodiments 2356-2366, wherein expression of DKK4 remains about the same or is increased.
2368. The method of any one of Embodiments 2356-2367, wherein expression of one or more of nucleic acids in BCAT_GDS748-UP or Table GS1 independently remains about the same or is increased.
2369. The method of any one of Embodiments 2356-2368, wherein expression of one or more of nucleic acids in BCAT.100-UP.V1-UP or Table GS2 independently remains about the same or is increased.
2370. The method of any one of Embodiments 2356-2369, wherein expression of one or more of nucleic acids in HALLMARK_WNT_BETA_CATENIN_SIGNALING or Table GS3 independently remains about the same or is increased.
2371. The method of any one of Embodiments 2356-2370, wherein expression of one or more of nucleic acids in RASHI_RESPONSE_TO_IONIZING_RADIATION_1 or Table GS4 independently remains about the same or is increased.
2372. The method of any one of Embodiments 2356-2371, wherein expression of one or more of nucleic acids in REACTOME_RRNA_PROCESSING or Table GS5 independently remains about the same or is increased.
2373. The method of any one of Embodiments 2356-2372, wherein expression of one or more of nucleic acids in HALLMARK_MYC_TARGETS_V1 or Table GS6 independently remains about the same or is increased.
2374. The method of any one of Embodiments 2356-2373, wherein expression of one or more of nucleic acids in HALLMARK_MYC_TARGETS_V2 or Table GS7 independently remains about the same or is increased.
2375. The method of any one of Embodiments 2356-2374, wherein expression of one or more of nucleic acids in HALLMARK_OXIDATIVE_PHOSPHORYLATION or Table GS8 independently remains about the same or is increased.
2376. The method of any one of Embodiments 2356-2375, wherein expression of one or more of nucleic acids in HALLMARK_E2F_TARGETS or Table GS9 independently remains about the same or is increased.
2377. The method of any one of Embodiments 2356-2376, wherein expression of one or more of nucleic acids in HALLMARK_TNFA_SIGNALING_VIA_NFKB or Table GS10 independently remains about the same or is increased.
2378. The method of any one of Embodiments 2356-2377, wherein expression of CXCL12 independently remains about the same or is decreased.
2379. The method of any one of Embodiments 2356-2378, wherein BCAT_GDS748-UP is not enriched or is positively enriched.
2380. The method of any one of Embodiments 2356-2379, wherein BCAT.100-UP.V1-UP is not enriched or is positively enriched.
2381. The method of any one of Embodiments 2356-2380, wherein HALLMARK_WNT_BETA_CATENIN_SIGNALING is not enriched or is positively enriched.
2382. The method of any one of Embodiments 2356-2381, wherein RASHI_RESPONSE_TO_IONIZING_RADIATION_1 is not enriched or is positively enriched.
2383. The method of any one of Embodiments 2356-2382, wherein REACTOME_RRNA_PROCESSING is not enriched or is positively enriched.
2384. The method of any one of Embodiments 2356-2383, wherein HALLMARK_MYC_TARGETS_V1 is not enriched or is positively enriched.
2385. The method of any one of Embodiments 2356-2384, wherein HALLMARK_MYC_TARGETS_V2 is not enriched or is positively enriched.
2386. The method of any one of Embodiments 2356-2385, wherein HALLMARK_OXIDATIVE_PHOSPHORYLATION is not enriched or is positively enriched.
2387. The method of any one of Embodiments 2356-2386, wherein HALLMARK_E2F_TARGETS is not enriched or is positively enriched.
2388. The method of any one of Embodiments 2356-2387, wherein HALLMARK_TNFA_SIGNALING_VIA_NFKB is not enriched or is positively enriched.
2389. The method of any one of the preceding Embodiments, wherein a comparison (e.g., reduced, increased, enriched, negatively enriched, positively enriched, etc.) is to a reference assessment prior to any administration or delivery.
2390. The method of any one of the preceding Embodiments, wherein a comparison (e.g., reduced, increased, enriched, negatively enriched, positively enriched, etc.) is to a reference assessment of a sample prior to any administration or delivery.
2391. The method of any one of the preceding Embodiments, wherein a comparison (e.g., reduced, increased, enriched, negatively enriched, positively enriched, etc.) is to a reference assessment at or during an administration or delivery.
2392. The method of any one of the preceding Embodiments, wherein a comparison (e.g., reduced, increased, enriched, negatively enriched, positively enriched, etc.) is to a reference assessment of a sample collected at or during an administration or delivery.
2393. The method of any one of the preceding Embodiments, wherein a comparison (e.g., reduced, increased, enriched, negatively enriched, positively enriched, etc.) is to a reference assessment after an earlier administration or delivery.
2394. The method of any one of the preceding Embodiments, wherein a comparison (e.g., reduced, increased, enriched, negatively enriched, positively enriched, etc.) is to a reference assessment of a sample collected after an earlier administration or delivery.
2395. The method of any one of the preceding Embodiments, wherein a comparison (e.g., reduced, increased, enriched, negatively enriched, positively enriched, etc.) is to a reference assessment after an administration or delivery of a reference agent.
2396. The method of any one of the preceding Embodiments, wherein a comparison (e.g., reduced, increased, enriched, negatively enriched, positively enriched, etc.) is to a reference assessment of a sample collected after an administration or delivery of a reference agent.
2397. The method of any one of Embodiments 2395-2396, wherein a reference agent is a therapeutic agent.
2398. The method of any one of Embodiments 2395-2396, wherein a reference agent is an inactive control agent.
2399. The method of any one of Embodiments 2395-2398, wherein the administration, delivery and/or assessment is conducted under comparably.
2400. An agent, compound, or composition, prepared and/or characterized by a method of any one of the preceding Embodiments.
2401. An agent, compound, or composition of any one of the preceding Embodiments, prepared and/or characterized by a method of any one of the preceding Embodiments.

### EXEMPLIFICATION

Those skilled in the art appreciate that various technologies are available for manufacturing and assessing provided agents including various peptides such as stapled peptides in accordance with the present disclosure, for example, many technologies for preparing small molecules and peptides can be utilized to prepare provided agents, and various assays are available for assessing properties and/or activities of provided agents. Described below are certain such useful technologies. As demonstrated herein, in some embodiments, it is confirmed that provided technologies can exhibit nanomolar cell-based activity in protein-protein interaction (PPI), transcriptional regulation, proliferation assays, etc. In some embodiments, it is confirmed that provided technologies possess favorable pharmacokinetic properties. In some embodiments, in vivo dosing of provided technologies confirms on-target pharmacodynamic modulation of 3-catenin activity and strong anti-tumor activity in multiple human xenograft models, which confirm that provided technologies are useful for treating various conditions, disorders or diseases as described herein.

### Example 1. Peptide Synthesis

Among other things, peptides can be prepared using various peptide synthesis technologies in accordance with the present disclosure. In many embodiments, peptides were prepared using Fmoc-based synthesis, often on suitable solid phase. For various stapled peptides, amino acid residues were stapled through suitable chemistry, e.g., olefin metathesis for amino acids that comprise olefin groups. Those skilled in the art appreciates that other suitable technologies may also be utilized for stapling in accordance with the present disclosure, e.g., those described in WO/2019/051327, WO/2020/041270, etc., the peptide staples and technologies for preparing peptides are incorporated herein by reference.

For example, in some embodiments, peptides were synthesized on a Liberty Blue peptide synthesizer with 1 M DIC in DMF and 1 M Oxyma in DMF using standard Liberty Blue conditions on either Rink Protide amide resin (primary carboxamides), ethyl indole AM resin (ethyl amides), amino alcohol 2-chlorotrityl resin (amino alcohols), or Wang resin with the C-terminal amino acid pre-loaded (carboxylic acids). Single coupling was used for all amino acids, save for residues following a stapling amino acid, and B5, which were double coupled. Final Fmoc deprotection was performed on the N-terminal residue, and capping, e.g., acetate capping, was performed by treating the resin with a suitable capping agent, e.g., 5% acetic anhydride, 2.5% diisopropylethylamine and 92.5% NMP for acetate capping, at room temperature for 30 min. Non-acetate amide caps were appended with suitable amounts of reagents, e.g., five equivalents of a carboxylic acid, five equivalents of DIC, and five equivalents of Oxyma in a suitable solvent, e.g., DMF.

Lactam staples and triazole staples were closed prior to olefin metathesis. Lactam staples were generated by incorporating the amino-containing residue as an Alloc-protected amino acid, and the carboxylate-containing residue as an allyl-protected amino acid. Alloc/allyl deprotection was performed by treating the peptide with 10 mol % Pd(Ph₃P)₄, plus ten equivalents of either morpholine, phenylsilane, or dimethyl barbituric acid, in dichloroethane at room temperature for 1 h. Lactam formation was performed by treating the resin with 10 equivalents of Oxyma and 10 equivalents of DIC at 40° C. for 2 h, then draining and washing the resin with DMF.

Triazole staples were generated by incorporating both the azide-containing amino acid and alkyne-containing amino acid during the linear synthesis of the peptide. Triazole ring closure was performed by treating the acylated, linear peptide with copper (II) sulfate (2 equivalents) and sodium ascorbate (2 equivalents) in a mixture of tert-butanol/water (2/1). This mixture was heated in a microwave at 80° C. for 30 min, and then the resin filtered off, followed by washing with DMF and methanol.

Olefin metathesis was performed by treating peptides with suitable metathesis catalysts under suitable conditions, in some embodiments, optionally with multiple cycles, e.g., four cycles, of 30 mol % Grubbs' first generation catalyst (CAS 172222-30-9) in dichloroethane at 40° C. for 2 h, and washing the resin with dichloroethane after each treatment.

Peptide staple hydrogenation was performed by treating the resin with fresh 30 mol % Grubbs' first generation catalyst (CAS 172222-30-9) in 1,2-dichlorobenzene. Triethylsilane (50 equiv) was added, and the resin was placed in a heated shaker at 50° C. overnight, then washed with dichloroethane.

Peptide cleavage was performed by treating resin with 95% trifluoroacetic acid and 5% triisopropylsilane for 1 h, and precipitation of the crude peptide in diethyl ether. Purification was performed by preparative HPLC with MS detection and a Waters XSelect CSH C18 column using water with 0.1% formic acid and acetonitrile with 0.10% formic acid. Typically, if isomers were identified and separated by HPLC purification they were isolated and tested separately by elution peaks (e.g., UV at 220 nm), otherwise peptides were isolated (often based on HPLC peaks) and tested as combinations (all peptides within a single HPLC peak were typically tested together in a single composition).

Amino acids suitable for synthesis are commercially available or can be prepared in accordance with the present disclosure. Certain amino acids and their preparations are described in the priority applications, WO 2022/020651 or WO 2022/020652, e.g., preparation of (S)-2-((((9H-fluoren-9-yl)methoxy)carbonyl)amino)-3-(2-(tert-butoxycarbonyl)phenyl)propanoic acid, tert-butyl (S)-3-(2-((((9H-fluoren-9-yl)methoxy)carbonyl)amino)-3-(benzyloxy)-3-oxopropyl)benzoate, TfeGA, etc., the amino acids and their preparations, including methods, reagents, intermediates, etc., of each of which are independently incorporated herein by reference.

Certain peptide preparations are presented below as examples.

Compounds with staples bridging substituted glutamine residues between AA7 and AA14 were synthesized in the following manner: Fmoc-BztA-Glu(OAllyl)-protide resin was synthesized on a Liberty Blue as described above. The allyl group was deprotected by treating with 10% Pd(Ph₃P)₄ and 10 equivalents phenylsilane in DCE for 1 h at room temperature. A mono-alloc protected diamine was coupled to the deprotected Glu residue by treating the resin with 4 equivalents of the protected diamine, 4 equivalents of DIC, and 4 equivalents of Oxyma in DMF at 40° C. for 2 h. The resin was then washed with DMF, and loaded back into the Liberty Blue, and the linear peptide sequence with Glu(OAllyl) at position 7 was completed. The resin was acetyl capped as described above. Alloc/allyl deprotection was performed by treating the peptide with 10 mol % Pd(Ph3P)4, plus ten equivalents of morpholine, and lactamization was performed by treating the resin with 10 equivalents of DIC and 10 equivalents of Oxyma in DMF at 40° C. Ring closing metathesis, cleavage and purification were performed as described above.
I-45, I-46, I-47, I-48, I-49, I-50, I-51, I-52, I-53, I-54: Compounds with staples between lysine residues at positions 7 and 14 were generated in the following manner: The linear sequence was synthesized on a Liberty Blue as described above, incorporating Fmoc-Lys(ivDde)-OH at positions 7 and 14. After acetyl capping and olefin metathesis, the ivDde groups were removed by treating with two cycles of 5% hydrazine in DMF at 40° C. for 30 min, then washing with DMF. The resin was then treated with two equivalents of a diacid, 5 equivalents of DIC, and 5 equivalents of Oxyma in DMF at 40° C. for 2 h. The resin was then washed with DMF, and DCE, and cleaved and purified as described above.
I-303, I-517, I-518: Biotinylated peptides were generated by incorporating Fmoc-Lys(ivDde)-OH in the linear sequence. After acetyl capping and olefin metathesis, the ivDde group was removed by treating with two cycles of 5% hydrazine in DMF at 40° C. for 30 min, then washing with DMF. The resin was then treated with 3 equivalents Biotin-PEG8-acid (CAS 2143964-62-7), 3 equivalents of HATU, 10 equivalents of diisopropylethylamine in DMF at 40° C. for 2 h. The resin was then washed with DMF, and DCE, and cleaved and purified as described above.
I-606, I-607: Peptides with azidolysine in the final sequence were generated by incorporating Fmoc-Lys(ivDde)-OH in the linear sequence. After olefin metathesis, the ivDde group was removed by treating with two cycles of 5% hydrazine in DMF at 40° C. for 30 min, then washing with DMF. The resin was then treated with three equivalents of 1H-imidazole-1-sulfonyl azide sulfate (CAS 1357503-23-1), 9 equivalents of diisopropylethylamine, and 0.5 equivalents of copper (II) sulfate pentahydrate in DMF at 40° C. for 3 h. The resin was then washed with DMF, water, DMF, and DCE, and cleaved and purified as described above.

Cysteine-containing staples were closed after olefin metathesis, peptide cleavage and purification. In a small vial the purified dicysteine peptide was dissolved in DMF, and 5 equiv. of the dibromo linker was added, followed by 100 mM ammonium bicarbonate pH 8 buffer, followed by DTT (10 mM). Upon completion of the stapling the crude reaction mixture was purified by preparative HPLC as described above.
I-469: Ac-PL3-OAsp-Npg-B5-Asp-3COOHF-Aib-Ala-Phe-Lys*3-PyrS2-2F3MeF-BztA-GlnR*3-Ala-protide resin was synthesized on Rink Amide resin and the lactam staple installed as above. A plastic syringe containing 200 mg of resin-bound peptide containing a free N-terminal amine was swollen in 0.5 mL DMF. To the swollen resin was added a solution of (2S)-4-(tert-butoxy)-2-hydroxy-4-oxobutanoic acid (85.5 mg, 0.45 mmol) in 0.5 mL DMF, 450 uL of 1 M DIC, and 450 uL of 1 M Oxyma. The syringe was shaken at room temperature for 90 minutes. The resin was then washed with DMF, DCM, MeOH, and again DCM, followed by drying under vacuum. The resin-bound peptide was swollen in 0.5 mL DCM. To the swollen resin was added 500 uL of 0.1 M DMAP in DCM, followed by a solution of PL3-Ac (88.75 mg 0.45 mmol) and DCC (92.8 mg 0.45 mmol) in DCM. The syringe was shaken at 40° C. for 3 hours. The resin was then washed with DMF, DCM, MeOH, and again DCM, followed by drying under vacuum. Ring-closing metathesis, peptide cleavage, and purification were then performed as described above.
I-427: Fmoc-R5-Asp-3COOHF-Aib-Ala-Phe-Lys*3-PyrS2-3Thi-BztA-GlnR*3-Ala-protide resin was synthesized on Rink amide resin and the lactam staple installed as described above. On a polypropylene syringe equipped with a porous polypropylene disc at the bottom, 0.05 mmol (˜0.18 g) of on-resin intermediate 1 was swollen on DCE for 15 min. Ring closing metathesis (RCM) between the side chains of R⁵ and PyrS2 was carried out under standard protocol (30 mol % Grubbs I catalyst, at 40° C., 2×2 h, in DCE). Afterwards the resin was washed with DCE 2×, DMF 2×, DCM, MeOH, and DCM. Next, the resin was swollen in DMF for 15 min, treated with 20% piperidine in DMF 2×25 min and washed with DMF 5× and NMP 2×. To the swollen resin was added a pre-activated mixture of Fmoc-AllylGly-OH (5 eq), Oxyma (5 eq) and DIC (5 eq) in NMP (0.4M). The mixture was shaken for 2-3 h at room temperature. Chloranil test indicated complete coupling. The resin was then washed with DMF 5×, and the above cycle was repeated with Fmoc-Asp(OtBu)-OH, Fmoc-αMePro-OH, and 4-pentenoic acid. A second RCM, now between the side chain of AllylGly and the 4-pentenoic acid N-terminus cap, was carried out under standard protocol (30 mol % Grubbs I catalyst, 40° C., 2×2 h, in DCE). The resin was then washed with DMF 4×, DCM 3×, MeOH, DCM, and dried under high vacuum.
   The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.5 mg).
I-429: The same experimental procedure described for I-427 was used in this synthesis. The only difference was the coupling of 5-hexenoic acid at the last step of the linear peptide synthesis. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.3 mg)
I-428: Starting with Fmoc-R5-Asp-3COOHF-Aib-Ala-Phe-Lys*3-PyrS2-3Thi-BztA-GlnR*3-Ala-protide resin, in a polypropylene syringe equipped with a porous polypropylene disc at the bottom, 0.05 mmol (˜0.18 g) of on-resin intermediate 1 was swollen on DCE for 15 min. Ring closing metathesis (RCM) between the side chains of R⁵ and PyrS2 was carried out by the standard protocol (30 mol % Grubbs I catalyst, at 40° C., 2×2 h, in DCE). After the resin was washed with DCE 2×, DMF 2×, DCM, MeOH, and DCM, it was swollen in 1,2-dichlorobenzene (DCB) for 15 min. The solvent was drained and to the resin was added ˜15 mg of Grubbs I catalyst as a solid. The syringe was closed with the moving plunger, followed by addition of triethylsilane (50 eq) and DCB (0.6 mL) to the mixture via needle. The syringe was shaken at 50° C. for 18 h to produce the corresponding i+7 reduced staple. The resin was then washed with DMF 4×, DCM 3×, MeOH, DCM and DMF. Next, the resin was swollen in DMF for 15 min, treated with 20% piperidine in DMF 2×25 min, and washed with DMF 5× and NMP 2×. To the swollen resin was added a pre-activated mixture of Fmoc-AllylGly-OH (5 eq), Oxyma (5 eq) and DIC (5 eq) in NMP (0.4M). The mixture was shaken for 2-3 h at room temperature. Chloranil test indicated complete coupling. The resin was then washed with DMF 5×, and the above cycle was repeated with Fmoc-Asp(OtBu)-OH, Fmoc-αMePro-OH, and 4-pentenoic acid. A second RCM, now between the side chain of AllylGly and the 4-pentenoic acid N-terminus cap, was carried out by the standard protocol (30 mol % Grubbs I catalyst, 40° C., 2×2 h, in DCE). The resin was then washed with DMF 4×, DCM 3×, MeOH, DCM, and dried under high vacuum. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (5.2 mg).
I-431: The same experimental procedure described for I-428 was used in this synthesis. The only difference was the coupling of 5-hexenoic acid at the last step of the linear peptide synthesis. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (0.85 mg).
I-425: Starting with Fmoc-R5-Asp-3COOHF-Aib-Ala-Phe-Lys*3-PyrS2-3Thi-BztA-GlnR*3-Ala-protide resin, in a polypropylene syringe equipped with a porous polypropylene disc at the bottom, 0.05 mmol (˜0.18 g) of an on-resin advanced intermediate precursor in which a staple (e.g., a (i, i+7) staple between R5 and Pyrs2) was not yet formed, was swollen in DMF for 15 min, treated with 20% piperidine in DMF 2×25 min, and washed with DMF 5× and NMP 2×. To the swollen resin was added a pre-activated mixture of Fmoc-AllylGly-OH (5 eq), Oxyma (5 eq), and DIC (5 eq) in NMP (0.4M). The mixture was shaken for 2-3 h at room temperature. Chloranil test indicated complete coupling. The resin was then washed with DMF 6×, and the above cycle was repeated with Fmoc-Asp(OtBu)-OH, Fmoc-αMePro-OH, and 4-pentenoic acid. Simultaneous RCM between the side chain of AllylGly and the 4-pentenoic acid N-terminus cap, as well as the side chains of amino acids R5 and PyrS2, was carried out by the standard protocol (30 mol % Grubbs I catalyst, 40° C., 4×2 h, in DCE). Afterwards, the resin was washed liberally with DCE, DCM, DMF, MeOH and DCM, and dried under high vacuum for 3 to 4 h. The resin was swollen in 1,2-dichlorobenzene (DCB) for 15 min. The solvent was drained and to the resin was added ˜15 mg of Grubbs I catalyst as a solid. The syringe was closed with the moving plunger, followed by addition of triethylsilane (50 eq) and DCB (0.6 mL) to the mixture via needle. The syringe was shaken at 50° C. for 18 h to produce the corresponding fully reduced analogue. The resin was then washed with DMF 4×, DCM 3×, MeOH, DCM, and dried under high vacuum. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (2 mg).
I-426: The same experimental procedure described for I-425 was used in this synthesis. The only difference was the coupling of 5-hexenoic acid in the last step of the linear peptide synthesis. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.5 mg).
I-471: Starting with Fmoc-R5-Asp-3COOHF-Aib-Ala-Phe-Lys*3-PyrS2-3Thi-BztA-GlnR*3-Ala-protide resin, in a polypropylene syringe equipped with a porous polypropylene disc at the bottom, 0.05 mmol (˜0.18 g) of an on-resin advanced intermediate precursor in which a staple (e.g., a (i, i+7) staple between R⁵ and Pyrs2) was not yet formed, was swollen in DMF for 15 min, treated with 20% piperidine in DMF 2×25 min, and washed with DMF 5× and NMP 2×. To the swollen resin was added a pre-activated mixture of Fmoc-AllylGly-OH (5 eq), Oxyma (5 eq), and DIC (5 eq) in NMP (0.4M). The mixture was shaken for 2-3 h at room temperature. Chloranil test indicated complete coupling. The resin was then washed with DMF 6×. The above cycle was repeated with Fmoc-Asp(OtBu)-OH, Fmoc-αMePro-OH, and 2-(prop-2-en-1-yloxy)-benzoic acid. Simultaneous ring closing metathesis (RCM) between the side chains of AllylGly and the benzoyl-O-allyl N-terminus cap, as well as the side chains of amino acids R5 and PyrS2 was carried out by the standard protocol (30 mol % Grubbs I catalyst, 40° C., 3×3 h, in DCE). The resin was washed liberally with DCE, DCM, DMF, MeOH and DCM, and dried under high vacuum for 3 to 4 h. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (0.83 mg).
I-519: Starting with Fmoc-R5-Asp-3COOHF-Aib-Ala-Phe-Lys*3-PyrS2-3Thi-BztA-GlnR*3-Ala-protide resin, in a polypropylene syringe equipped with a porous polypropylene disc at the bottom, 0.05 mmol (˜0.18 g) of an on-resin advanced intermediate precursor in which a staple (e.g., a (i, i+7) staple between R⁵ and Pyrs2 was not yet formed), was swollen in DCE for 15 min. Ring closing metathesis (RCM) between the side chains of R⁵ and PyrS2 was carried out by the standard protocol (30 mol % Grubbs I catalyst, 40° C., 2×2 h, in DCE). The resin was then washed with DCE 3× and DMF 3×. Afterwards, the resin was swollen in DMF for 15 min, treated with 20% piperidine in DMF 2×25 min, and washed with DMF 5× and NMP 2×. To the swollen resin was added a pre-activated mixture of Fmoc-Dap(ivDde)-OH (5 eq), Oxyma (5 eq) and DIC (5 eq) in NMP (0.4M). The mixture was shaken for 2-3 h at room temperature. The resin was then washed with DMF 6×, and the above cycle was repeated with Fmoc-Asp(OtBu)-OH, Fmoc-αMePro-OH, and 5-hexenoic acid. The ivDde protecting group on the diamino propionic acid (Dap) side chain was removed by treating the DMF-swollen resin with a 5% solution of hydrazine in DMF, 2×20 min at 40° C. Afterwards, the resin was liberally washed with DMF and 2×NMP. To the swollen resin was added ortho-nitrobenzensulfonyl chloride (4 eq) and 2,4,6-collidine (4 eq) in NMP, and the reaction was shaken for 30 min at room temperature, to yield the desired N-activated intermediate. The resin was liberally washed with DMF. N-alkylation of the activated primary amine was carried out with allyl bromide (15 eq) and DBU (15 eq) in DMF, shaking the resin at room temperature for 2 days. The resin was liberally washed with DMF and 2×NMP. To push the N-alkylation reaction to completion, the resin was treated with allyl bromide (15 eq) and 2,6-lutidine (15 eq) in NMP at 110° C. for 30 min under microwave conditions. The resin was liberally washed with DMF and 2×NMP, and trial cleavage and analysis by LCMS showed complete reaction. The N-activating group (oNBS) was removed by treating the resin with mercaptoethanol (10 eq) and DBU (5 eq) in NMP (2×20 min at room temperature). The resulting secondary amine, at the side chain of Dap, was alkylated with benzyl bromide (10 eq) and 2,6-lutidine (15 eq), under microwave conditions at 110° C. 2×25 min. A second RCM between the side chains of Dap(allyl) and the 5-hexenoic acid N-terminus cap, was carried out by the standard protocol (30 mol % Grubbs I catalyst, 40° C., 2×2 h, in DCE). The resin was washed liberally with DCE, DCM, DMF, MeOH and DCM, and dried under high vacuum for 3 to 4 h. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (0.55 mg).
I-520: The same experimental procedure described for I-519 was used in this synthesis. The only difference was the acylation (instead of alkylation) of the produced secondary amine at the side chain of Dap using benzoic acid (5 eq), Oxyma (5 eq), and DIC (5eq) in NMP, at room temperature for 2-3 h. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (0.62 mg).
I-564: The same experimental procedure described for I-519 was used in this synthesis, with two important changes. First was the acylation (instead of alkylation) of the produced secondary amine at the side chain of Dap using pivaloyl chloride (7 eq) and NMM (10 eq) at 77° C. for 15 min under microwave conditions. Second, the resin was swollen in 1,2-dichlorobenzene (DCB) for 15 min followed by, after solvent draining, addition of ˜15 mg of Grubbs I catalyst as a solid. The syringe was closed with the moving plunger and triethylsilane (50 eq) and -0.6 mL of DCB were added to the mixture via needle. The syringe was shaken at 50° C. for 18 h to produce the corresponding fully reduced analogue. The resin was then washed with DMF 4×, DCM 3×, MeOH, and DCM, and dried under high vacuum. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.36 mg).
I-565:The same experimental procedure described for I-564 was used in this synthesis. The only difference was the acylation of the produced secondary amine at the side chain of Dap using cyclohexanecarboxylic acid (5 eq), Oxyma (5 eq), and DIC (5eq) in NMP at room temperature for 2-3 h. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.19 mg).
I-562: A similar experimental procedure as described for I-519 was used in this synthesis, although with three important changes. First, 4-pentenoic acid was used as the N-terminus capping group. Second, N-alkylation of the secondary amine, at the side chain of Dap, was carried out with benzyl bromide (10 eq) and 2,6-lutidine (15 eq), under microwave conditions at 110° C., 2×25 min. Third, both alkene staples were simultaneously reduced. The resin was swollen in 1,2-dichlorobenzene (DCB) for 15 min, the solvent was drained, and to the resin was added ˜15 mg of Grubbs I catalyst as a solid. The syringe was closed with the moving plunger and triethylsilane (50 eq) and DCB (0.6 mL) were added to the mixture via needle. The syringe was shaken at 50° C. for 18 h to produce the corresponding fully reduced analogue. The resin was then washed with DMF 4×, DCM 3×, MeOH, and DCM, and dried under high vacuum. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (0.44 mg).
I-563: The same experimental procedure described for I-562 was used in this synthesis. The only difference was the acylation (instead of alkylation) of the produced secondary amine at the side chain of Dap using benzoic acid (5 eq), Oxyma (5 eq), and DIC (5eq) in NMP at room temperature for 2-3 h. The peptide was cleaved off the resin with 3 ml TFA/H2O/TIPS (95:2.5:2.5) for 2 h at room temperature, then precipitated by cold ethyl ether, and the obtained residue was applied to a reverse-phase HPLC column to afford, after lyophilization of the pure fractions, the titled compound as a white powder (1.19 mg).

Mass spectrometry was performed as follows: 2 uL of a 200 uM solution of a peptide in DMSO was injected on a Waters Acquity UPLC-MS system with a 2.1×50 mm, 1.7 μM CSH C18 column at 40° C., using a gradient of 95/5 water/acetonitrile to 5/95 water/acetonitrile over 7 minutes, flow rate=0.6 mL/min. Product peaks were analyzed in both positive and negative ionization mode.

### Example 2. Provided Technologies can Provide Improved Properties and/or Activities

In some embodiments, solubility was assessed. In some embodiments, a useful protocol is presented below as an example: 50 uM peptide was incubated in 99.5% PBS/0.5% DMSO at 37° C. for 15 min. After ultracentrifugation of the PBS solution, the supernatant was analyzed by HPLC and compared to an HPLC injection 50 uM peptide DMSO solution. Solubility was determined by: [(Area of PBS peak)/(Area of DMSO peak)]*50 uM. In some embodiments, provided agents, e.g., stapled peptides, have a solubility of about or at least about 1-50, 10-50, 10, 20, 30, 40, or 50 uM as measured using such a protocol.

In some embodiments, LogD of provided agents, e.g., stapled peptides, were assessed. In some embodiments, shake flask LogD was assessed using the following procedure as an example. In some embodiments, certain agents, e.g., stapled peptides, have a shake flask LogD of about 0-3, 0.1-2.5, 0.5-2, 1-2, 1.5-2, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3.

### Instruments Required:

EP Motion
4titude Plate Sealer
Sample Scanner
Hamilton Decapper
Eppendorf Centrifuge
Eppendorf Shaker
Sonicator
Agilent Single Quad HPLC-MS

### Materials Needed:

Eppendorf 384 well 100 uL volume plate
Eppendorf 96 Well 1 mL Plate
Eppendorf 96 Well 500 uL Plate
EP Motion
EP Motion 50 uL, 300 uL, 1000 uL tips.

### Preparation for Plate Generation:

1. Take a tray of aliquots from compound management.
2. Use a maximum of 45 compounds and reserve the last 3 spots for aliquots of standard.
3. Scan the plate of aliquots on the SampleScan
4. Take the scanned values and generate an excel file
5. Generate a .csv file from the outputted excel
   a. This .csv should contain two columns
      i. First Column: Sample Location
      ii. Second Column: Sample Name
6. Spin compounds down in centrifuge for 15s, at 3000 rpm.
7. De-cap aliquots using the Hamilton decapper.
8. Take the tray of aliquots up to the EP Motion.

### Plate Generation:

1. On the EP Motion select:
   a. Home>Chemistry>logDPart1v1_100 mL_NoSTD
2. Place aliquots, tips, plate (96-well 1 mL Eppendorf Plate,) and reservoirs according to instrument.
a. Reservoirs contain presaturated Octanol pH 7.4, and presaturated buffer pH 7.4.
3. Make sure the total number of samples reads 48.
4. Select run, and ensure “Detect Volumes” is selected, you can unselect “check tips” and “labware placement.”
5. Run method.
6. Remove completed 96-well plate. Clean up the EP Motion.
7. Take completed plate to compound management and turn on the 4titude plate sealer.
   a. Wait until plate sealer displays a temperature of 170 C.
   b. Place silver sheet over plate, utilize gold holder to keep silver sheet in place.
   c. Select operate, place plate in holder with holder in place. Press operate again.
   d. Use a roller to firmly seal the plate once it has been ejected.
8. Invert plate on side, place on Eppendorf shaker for 1 hour at 2000 rpm.
9. Remove Plate, sonicate for 10 minutes.
10. Centrifuge at 3000 rpm for 10 minutes.

### Plate Generation: Final

1. On the EP Motion select:
   a. Home>Chemistry>logDPart2v1-80 mL
2. Place aliquots, tips, plates (96-well 1 mL Eppendorf Plate, 96-well 500 mL Eppendorf Plate, 384-well 100 uL plate (Final),) and reservoirs according to instrument.
   a. Reservoirs contain (50/50) presaturated Octanol pH 7.4/DMSO, DMSO, Acetonitrile, and presaturated buffer pH 7.4.
3. Make sure the total number of samples reads 48.
4. Select run, and ensure “Detect Volumes” is selected, you can unselect “check tips” and “labware placement.”
5. Run method.
6. Remove completed 96-well plates and final 384-well plate. Clean up the EP Motion.
7. Seal both 96-well plates with a rubber plate seal, store in 4 C freezer.
8. Take completed final plate to compound management and turn on the 4titude plate sealer.
   a. Wait until plate sealer displays a temperature of 170 C.
   b. Place pierceable silver sheet over plate, utilize gold holder to keep silver sheet in place.
   c. Select operate, place plate in holder with holder in place. Press operate again.
   d. Use a roller to firmly seal the plate once it has been ejected.
9. Head over to Agilent HPLCMS.

### Plate Programming:

1. Open Chemstation on the Agilent HPLCMS
2. Ensure buffers C (Water with 0.1% Formic Acid,) D (Acetonitrile with 0.1% Formic Acid,) and wash (MeOH,) are full.
3. Hit the green on button to allow instrument sufficient time to equilibrate while the sequence is programmed.
4. Click the sequence button in the top drop-down menu:
   a. Select new sequence
   b. Save sequence as yyyymmdd_sol.
5. From the sequence menu:
   a. Select import samples
   b. Click browse and find the .csv file created earlier.
   c. Click next, then click finish.
6. Open the Sequence:
   a. The only columns that should be filled are the location:
   b. And the Sample Name:
7. Double click on the method box:
   a. Select 10_mincd 60-95
8. Enter 10 for the injection volume, 10 uL will be injected
9. In the injection number enter 2, for 2 injections per well.
10. Now highlight the columns containing method, injection volume, and injection number and drag down to the bottom of the sequence. Hold Ctrl and right click, select fill down.
11. Insert a blank sample in the 1′ and last slot.
   a. For sample location enter D1B-D1
   b. For sample name enter Blank
   c. Enter the same method 10_mincd 60-95.
   d. Enter 10 for injection volume
   e. Enter 4 for number of injections
12. Right click on the widget for the Mass Spec and enter the mass range for the compounds selected to run.
13. Click start.

### Data Processing:

1. Open the offline version of the Chemstation software, select the desired sequence by date.
2. Double-click on the line containing the compound of interest. This will bring up the chromatogram.
   a. Select the delimiting tool to remove any automatic integration.
   b. Select the Average Chromatogram tool for mass and drag it over the peaks of interest.
   c. Find the expected mass.
   d. Go back to the peak delimiting tool again and drag it over the peak containing the mass of interest to integrate the peak.
   e. Perform the same for the second preparation of the same compound.
3. Export the integrated compound results as a pdf.
4. Take the integrated area and enter it in the entry sheet in the excel.
5. Once all results are entered, the functions within the excel will automatically calculate the Average Logd and Standard deviation.
   a. To account for dilutions throughout the plate creation the final calculation for the Logd looks like this:
      i. Logd=log ((Octanol peak*40)/(Buffer peak*2))
      ii. Average Logd is the average of the calculated Logd values of peak 1 and 2, as is the standard deviation from the calculated Logd values of peak 1 and 2.
      iii. Special cases: Logd only seen in the Octanol phase is denoted as >0, Logd only seen in the buffer phase is denoted as <0.
      iv. If compound is not seen in either phase, it likely indicates a solubility problem. Generally noted as Div/0! and observation included in the notes section.

Certain results are presented herein as examples.

### Example 3. Various Provided Peptides can Bind to Beta-Catenin

As those skilled in the art will appreciate, many technologies can be utilized in accordance with the present disclosure to assess binding to targets such as beta-catenin. Certain useful technologies and results are described below as examples.

In some embodiments, an assay is fluorescence polarization. A useful protocol is described below as an example.

Fluorescence polarization IC50: Using the Mosquito (SPT) peptide solutions were 3-fold serially diluted in 90% DMSO and 40 nL of titrated peptide was added into 20 uL buffer (50 mM HEPES, pH 7.5, 125 mM NaCl, 2% glycerol, 0.5 mM EDTA, 0.05% v/v pluronic acid) for final concentrations of 10 uM to 5 nM plated by Multidrop™ Combi (Thermo Scientific) into a black polystyrene 384-well plate (Corning). Probe solution (10 nM full-length B-Catenin (Uniprot ID P35222), mixed with 10 nM 5FAM labeled TCF4 residues 10-53 (Uniprot ID Q9NQB0) peptide in buffer) was prepared and 20 uL per well was plated using a Multidrop™ Combi (Thermo Scientific). The plate was incubated protected from light for 60 minutes at 20° C. prior to read. Reads were performed on a CLARIOstar plate reader (BMG Labtech) in duplicate, and data were fitted to a 1:1 binding model with hill slope using an in-house script. All provided concentrations are final concentrations. Certain results were presented in Table E1 below as examples.

In some embodiments, binding to beta-catenin may be measured by surface plasmon resonance (SPR). A useful protocol is described below as an example. Various agents, e.g., those presented in E2 as examples, demonstrated binding to beta-catenin, in some embodiments, with low or sub-nM Kd; other values can and in various cases were also assessed, e.g., t_{1/2}.

Peptides at 10 mM concentration in DMSO are diluted into Biacore™ running buffer (50 mM Tris pH 8.0, 300 mM NaCl, 2% glycerol, 0.5 mM TCEP, 0.5 mM EDTA, 0.005% Tween-20, 0.09% DMSO) to afford an appropriate dilution range. These diluted peptide samples are then assayed on a Biacore™ S200 using the Biacore™ Biotin CAPture Kit (GE Healthcare) which had been functionalized with biotinylated B-Catenin residues 134-665 (Uniprot ID P35222). Results were analyzed using the Biacore™ Insight Evaluation Software, fitting to a 1:1 binding model.

### Example 4. Provided Technologies can Modulate Interactions with Beta-Catenin in Cells

Various technologies may be utilized to assess properties and/or activities of provided compounds, e.g., stapled peptides, in cells. In some embodiments, a useful assay is Nano-BRET target engagement assay that assesses beta-catenin/TCF4 engagement. A useful protocol is described below as an example.

On Day 1, HEK293 cells were seeded. Cells at ˜70% confluency were utilized. Trypsinize cells without washing with PBS (e.g. 5 ml trypsin/75 flask for 2-5 min @ Rm Temp). Quench trypsin with 10 mL MEM media. Transfer cells to a falcon tube. Spin down @ 250 g for 5 minutes at room temperature. Discard supernatant. Gently re-suspend the cells in 10 mL MEM media. Count the cells twice and calculate how many cells were needed. Plate Parental HEK293 Cell Line at 7 M cells/12 ml/75 cm2 flask using MEM media. Rock plate a couple of times to disperse cells evenly. Incubate at 37° C., 5% CO₂ for 5 hours. Cells should be evenly spread and about 70% confluent after, e.g., 5h.

Transfection of Nano-BRET constructs (B-cat-Halo & TCF4-Luc): Allow Fugen-HD transfection reagent to reach room temperature. Mix by inverting tube, if precipitate is visible, warm up to 37° C. and them cool to room Temp. Check flasks under microscope for confluency of cells (70-80%). Add LiCl to flask containing cells (LiCl 30 mM working concentration—LiCl can be a GSK3 inhibitor and reduce beta-catenin degradation). Prepare the transfection mix in a tube containing Assay media based on the manufacturer instruction (see below table for an example):

### Transfection Mix Preparation

| | | # of | | DNA | FuGene | Opti-MEM |
|---|---|---|---|---|---|---|
| # | Constructs | Flasks | Ratio | (ug) | (6 ul/w) | (ul) |
| 1-a | Bcat-Halo | 1 | 4 | 12.8 | 48 | 736 |
| 1-b | TCF4-Luc | 1 | 1 | 3.2 | | |

Add FuGene last and gently mix. Don't vortex. Incubate transfection mix at RT or 10-15 minutes. If more than one target pair is going to be tested, calculate the amounts of transfection mix using the above table for other construct pairs. Gently add 700 uL of transfection mix per flask and gently rock the plate a couple of times. Incubate cells at 37° C., 5% CO2 for 18-24 hours.

On Day 2, transfected cells were harvested and re-plated in 384-well plates with media and compounds pre-dispensed in the wells. Dispense 20 uL of 30 mM LiCl containing assay media in all wells of a 384-well plate. In some embodiments, a liquid handling system was utilized to prepare a compound plate with a top concentration of 10 mM and serially diluted in a 1:3 manner to a lowest concentration of 13 uM. Dispense 80 nL of these compound series into the 20 uL of media pre-dispensed in the plates. This created a 2× concentration in the wells that was further diluted once cells were added.

While compound dilutions and dispenses were being made, collect media from transfected cell flask in a Falcon tubes. This was to harvest the floaters as they may still be viable and transfected. Trypsinize cells without washing with PBS (5 ml trypsin/Flask). Quench trypsin with 5 mL of MEM media. Collect cells and add to falcon tube. Wash the flask with 5-10 mL of MEM media and add to falcon tube. Spin down @ 250 g for 5 minutes at room temperature. Discard supernatant. Gently re-suspend cells in 5 mL Assay media (optionally containing LiCl). Count the cells twice and calculate the average count. Dilute HaloTag® NanoBRET™ 618 Ligand 1:500 in cell dilution. Dispense 20 uL of cell suspension per each well for all except one column of 384-well plate (5,000 cells/40 uL/well) (use plate such as Corning Solid White Flat Bottom TC-treated plate). For final column add 20 uL of cells containing equivalent amounts of DMSO. LiCl at 30 mM concentration. This cell dispense to the 20 uL of compound containing media brings the compound concentrations to our desired final working dilutions. Incubate at 37° C., 5% CO2 overnight.

On Day 3, fluorescence was read with Nano-BRET substrates. Remove plates from incubator to allow to reach to RT (30 min). Also equilibrate CTG reagent to room temperature. Dilute Nano-BRET substrate 1:100 in Assay media. Add 10 uL of diluted substrate to each well and shake for 30 seconds. Read on ClarioSTAR or GloMAX right away (within 10 min). Donor emission @ 460 nm. Acceptor emission @618 nm. Use the same plate to measure cell viability (Cell Titer-Glo-2.0 (CTG) Viability test). After reading BRET signal, add CTG reagent to each well at 1:2 ratio and shake on orbital shaker for 2 min. Incubate at Rm Temp for 10-30 min. Read luminescence on ClarioSTAR or GloMAX. Analysis was performed using non-linear regression in R, Log(inhibitor) vs. response with a two parameter Hill function, and a high control (cells with ligand) and low control (cells without ligand), to measure absolute IC50 (AbsIC50=X[50]) of each compound.

Certain results were presented in Table E1 as examples.

Reporter IC50: Activities of provided technologies were also confirmed in TCF reporter assay as described below. Those skilled in the art will appreciate that other suitable reagents may be utilized and various parameters may be adjusted.

On Day 1, cultured cells (e.g., DLD1) in flasks that were no more than about 60-70% confluent were washed with PBS and typsinized in 3 mL/T75 until cells were free floating. Cells were spun down for 5 minutes at 1100RPM. After spinning, the supernatant was gently aspirated and cells were resuspended in 10 mL assay media (4% FBS RPMI or 20% FBS RPMI, depending on desired serum concentration). Cells were counted twice using a Countess cell counter, counts were averaged, and the cell concentration was adjusted. The desired seeding density was 2500 cells/well in 40 uL assay media. Using a Multidrop Combi, the cells were plated in columns 1-22 in 384 well, white solid-bottom plate. Cell-free assay media was added to columns 23 and 24. Assay plates were incubated at 37° C., 5% CO2 overnight on the top shelf (back) of an incubator.

On Day 2, compounds were added. Stock solution was 10 mM. A liquid handling system was used to prepare the compound dilution and dispense compound into assay plates. The compounds were serially diluted 1/2 or 1/3 (depending on desired assay conditions) in 90% DMSO to create a 7 point dose curve. From compound plate, 80 nL of compound were dispensed directly into wells of the assay plates to create a dose curve starting at 20 uM and ending at either 313 nM (1/2 dilution) or 27 nM (1/3 dilution). Untreated, control wells received 90% DMSO only. Assay plates were incubated at 37° C., 5% CO2 overnight on the top shelf (back) of an incubator.

On Day 3, viability was read using Cell-Titer Fluor (CTF, Promega) and TCF activity was read using BrightGlo (Promega). CTF was mixed to 5× concentration using 35 uL substrate to 14 mL buffer. Warmed CTF was added directly to uncooled assay plates using Multidrop Combi, 10 uL/well in columns 1-23. Assay plates were incubated at 37° C., 5% CO2 on the top shelf (back) of an incubator for 2 hours and then removed. Removal of assay plates from incubator was staggered in 5 min intervals. Plates were cooled for 40 min, protected from light, and read using GloMax CTF program (High Sensitivity).

After reading CTF, room temperature BrightGlo was added to room temperature assay plates using Multidrop Combi, 35 uL/well in columns 1-23. The plates were incubated at room temperature for 2 minutes, protected from light. Then plates were read using a ClarioStar, end point luminescence readout.

Analysis was performed using non-linear regression in R, Log(inhibitor) vs. response with a two parameter Hill function, and a high control (DMSO treated cells) and low control (Cell-free wells), to measure absolute IC50 (AbsIC50=X[50]) of each compound.

For various agents, e.g., certain stapled peptides in Table E2 or Table E3, low or sub-uM IC50 were observed. Certain results were presented in Table E1 as examples.

COLO320DM proliferation assay IC50: In some embodiments, inhibition of cell proliferation by provided technologies were assessed using cell lines related to or from certain conditions, disorders or diseases. In some embodiments, cell proliferation was assessed in COLO320DM cells. In some embodiments, assessment was performed using the following procedure: On Day 1, cultured COLO320DM cells in a T75 flask were trypsinized in 3 mL of 0.250% trypsin/EDTA for 5 min and quenched with 10 mL RPMI-1640+4% HI FBS assay media. The cells were spun down at 1200 rpm for 5 min, the cell pellet collected and re-suspended at 5000 cells/mL in assay media. Using a Combi liquid handler, cells were dispensed (50 uL, 250 cells/well) into three 384 well plates. Plates were incubated at 37° C., 5% CO2 for 18-22 h. On day 2, compounds were added. A liquid handling system was used to prepare the compound dilution and dispense compound into assay plates. The compounds were serially diluted 1/2 in 90% DMSO to create a 7 point dose curve. From compound plate, 100 nL of compound were dispensed directly into wells of the assay plates to create a dose curve starting at 20 uM and ending at 313 nM. Assay plates were incubated at 37° C., 5% CO2 for 96 h. On day 6, assay plates were removed from the incubator and allowed to sit at room temperature for 30 min. Using a liquid handler, 20 uL of CellTiter Glo reagent was added to each well. The assay plates were shaken for 2 min and allowed to sit on the bench for 10-15 minutes. The assay plates were read using the CellTiter Glo protocol on a GloMax microplate reader, and the data analyzed using GraphPad Prism. Activities of various agents, including various stapled peptides in Table E2, were confirmed. Certain results are presented in Table E1 below.

### Table E1. Certain Data of Various Compositions as Examples.

Structural information and compositions of stapled peptides are described in Table E2.
1. Compound ID
2. beta-Catenin FP IC50 (nM): A≤50 nM; 50 nM<B≤200 nM; 200 nM<C≤750 nM; 750 nM<D≤1000 nM; E>1000 nM
3. NanoBRET Abs IC50 (uM): A≤1.5 uM; 1.5 uM<B≤3.0 uM; 3.0 uM<C≤10.0 uM; D>10.0 uM
4. DLD1 4% Abs IC50 (uM): “+”≤1.0 uM; 1.0 uM<“++”≤5.0 uM; “+++”>5.0 uM
5. COLO320DM Proliferation Abs IC50 (uM): “+”≤10.0 uM; 10.0 uM<“++”≤20.0 uM; “+++”>20.0 uM
6. Calculated Mass
7. Found m/z (positive mode)
8. Found m/z (negative mode)
9. C═C double bond (e.g., —CH═CH—) reduction to single bond (e.g., —CH₂—CH₂—). A: —CH═CH— in each staple reduced to —CH₂—CH₂—; B: —CH═CH— in C-terminal side staple reduced to —CH₂—CH₂— (see, e.g., see preparation of I-428 and I-432 as examples)

| 1 | 2 | 3 | 4 | 5 | 6 | 7 | 8 | 9 |
|---|---|---|---|---|---|---|---|---|
| I-1 | A | D | | | 1899.8 | 1901.4 | 1899.3 | |
| I-2 | A | | | | 1899.8 | 1901.3 | 1899.4 | |
| I-3 | A | C | | | 1899.8 | 1901.3 | 1899.4 | |
| I-4 | A | D | | | 1899.8 | 951.3 | 1899.5 | |
| I-5 | A | D | | | 1899.8 | 1901.3 | 1899.4 | |
| I-6 | A | D | | | 1899.8 | 951.2 | 1899.3 | |
| I-7 | A | D | | | 1956.8 | 1958.4 | 1956.5 | |
| I-8 | A | D | | | 1956.8 | 1958.4 | 1956.5 | |
| I-9 | A | C | | | 1975.9 | 1977.4 | 1975.4 | |
| I-10 | A | D | | | 1975.9 | 1977.3 | 1975.4 | |
| I-11 | A | B | | | 1975.9 | 1977.4 | 1975.5 | |
| I-12 | A | C | | | 1975.9 | 1977.3 | 1975.5 | |
| I-13 | A | C | | | 1975.9 | 1977.3 | 1975.4 | |
| I-14 | A | C | | | 1975.9 | 1977.4 | 1975.5 | |
| I-15 | A | B | | | 1975.9 | 989.2 | 1975.4 | |
| I-16 | A | C | | | 1975.9 | 989.3 | 1875.5 | |
| I-17 | A | D | | | 1923.8 | 1925.5 | 1923.3 | |
| I-18 | A | | | | 1923.8 | 1925.3 | 1923.3 | |
| I-19 | A | C | | | 1999.9 | 2001.4 | 1999.3 | |
| I-20 | A | | | | 1999.9 | 2001.3 | 1999.3 | |
| I-21 | A | B | | | 2061.9 | 2063.3 | 2061.3 | |
| I-22 | A | | | | 2061.9 | 2063.4 | 2061.4 | |
| I-23 | A | C | | | 1923.8 | 1925.2 | 1923.2 | |
| I-24 | A | C | ++ | | 1999.9 | 2001.3 | 1999.4 | |
| I-25 | A | C | | | 2061.9 | 1032.2 | 2061.3 | |
| I-26 | D | | | | 1980.9 | 1982.3 | 1980.3 | |
| I-27 | C | | | | 2056.9 | 2058.3 | 2056.4 | |
| I-28 | D | | | | 2118.9 | 2120.2 | 2118.4 | |
| I-29 | A | D | | | 1980.9 | 1982.3 | 1980.3 | |
| I-30 | A | D | | | 2056.9 | 2058.5 | 2056.5 | |
| I-31 | A | D | | | 2118.9 | 2120.3 | 2118.4 | |
| I-32 | A | A | | | 1989.9 | 1992.0 | 1990.1 | |
| I-33 | A | C | | | 1989.9 | 996.6 | 1990.1 | |
| I-34 | A | C | | | 1975.9 | 1977.9 | 1976.0 | |
| I-35 | D | | | | 1975.9 | 1977.9 | 1976.1 | |
| I-36 | C | | | | 1975.9 | 1977.9 | 1975.9 | |
| I-37 | A | C | | | 1975.9 | 1977.9 | 1976.2 | |
| I-38 | A | C | | | 1975.9 | 989.6 | 1976.3 | |
| I-39 | A | D | | | 1961.8 | 1964.0 | 1961.9 | |
| I-40 | E | | | | 1961.8 | 1963.9 | 1961.7 | |
| I-41 | C | | | | 1961.8 | 1963.9 | 1961.9 | |
| I-42 | A | B | | | 2060.9 | 2063.1 | 2061.1 | |
| I-43 | A | B | | | 2060.9 | 1032.2 | 2060.9 | |
| I-44 | D | | | | 2046.9 | 2049.0 | 2046.9 | |
| I-45 | A | D | | | 2179.0 | 2181.3 | 2179.4 | |
| I-46 | A | C | | | 2131.0 | 2133.4 | 2131.5 | |
| I-47 | A | D | | | 2145.0 | 2147.3 | 2145.4 | |
| I-48 | A | D | | | 2255.0 | 2257.6 | 2255.1 | |
| I-49 | B | C | | | 2255.0 | 2257.4 | 2255.4 | |
| I-50 | B | D | | | 2207.0 | 1105.4 | 2208.2 | |
| I-51 | A | C | | | 2159.0 | 2161.7 | 2159.7 | |
| I-52 | A | D | | | 2173.0 | 2175.5 | 2173.7 | |
| I-53 | B | D | | | 2283.0 | 2285.6 | 2283.9 | |
| I-54 | B | | | | 2283.0 | 2285.7 | 2283.6 | |
| I-55 | A | B | | | 2051.9 | 2053.9 | 2052.3 | |
| I-56 | A | C | | | 2037.9 | 2039.9 | 2037.8 | |
| I-57 | A | B | | | 2122.9 | 2125.0 | 2123.1 | |
| I-58 | B | D | | | 2108.9 | 2111.1 | 2108.8 | |
| I-59 | B | D | | | 2108.9 | 2111.1 | 2109.1 | |
| I-60 | A | C | | | 2180.0 | 2182.1 | 2180.1 | |
| I-61 | A | D | | | 2165.9 | 2168.2 | 2165.9 | |
| I-62 | A | C | | | 2077.9 | 2080.1 | 2078.2 | |
| I-63 | A | C | | | 2077.9 | 2080.0 | 2078.2 | |
| I-64 | A | B | ++ | ++ | 2073.9 | 2076.1 | 2074.0 | |
| I-65 | A | B | | | 2047.9 | 2050.0 | 2048.0 | |
| I-66 | A | A | | | 2074.9 | 1038.8 | 1036.8 | |
| I-67 | A | A | + | + | 2074.9 | 2076.6 | 2074.7 | |
| I-68 | A | B | | | 2137.0 | 1069.7 | 1067.8 | |
| I-69 | A | A | + | + | 2137.0 | 2138.7 | 2136.8 | |
| I-70 | A | A | + | + | 2103.0 | 2104.6 | 2102.8 | |
| I-71 | A | B | | | 2165.0 | 2166.7 | 2164.8 | |
| I-72 | A | B | | | 2090.9 | 2092.5 | 2090.6 | |
| I-73 | A | B | | | 2152.9 | 2154.6 | 2152.7 | |
| I-74 | A | D | | | 2101.9 | 2103.5 | 2101.6 | |
| I-75 | A | D | | | 2163.9 | 2165.5 | 2163.6 | |
| I-76 | A | C | | | 2064.9 | 2066.6 | 2064.6 | A |
| I-77 | A | D | | | 2127.0 | 2128.8 | 2126.8 | A |
| I-78 | A | D | | | 2105.9 | 2107.6 | 2105.8 | A |
| I-79 | B | | | | 2168.0 | 2169.8 | 2167.9 | A |
| I-80 | A | C | | | 2059.9 | 2061.6 | 2059.8 | |
| I-81 | B | C | | | 2046.9 | 2048.5 | 2046.6 | |
| I-82 | A | C | | | 2059.9 | 2061.5 | 2059.6 | |
| I-83 | C | D | | | 2046.9 | 2048.5 | 2046.6 | |
| I-84 | A | C | | | 2075.9 | 2077.6 | 2075.7 | |
| I-85 | B | D | | | 2062.9 | 2064.5 | 2062.6 | |
| I-86 | B | D | | | 2075.9 | 2077.7 | 2075.8 | |
| I-87 | C | | | | 2062.9 | 2064.6 | 2062.7 | |
| I-88 | A | D | | | 2116.9 | 2118.8 | 2116.8 | |
| I-89 | B | D | | | 2103.9 | 2105.7 | 2103.8 | |
| I-90 | B | D | | | 2116.9 | 2118.6 | 2116.7 | |
| I-91 | C | | | | 2103.9 | 2105.7 | 2103.7 | |
| I-92 | A | | | | 2236.0 | 1119.3 | 1117.3 | |
| I-93 | C | | | | 2248.0 | 1125.6 | 1123.4 | |
| I-94 | C | | | | 2248.0 | 1125.6 | 1123.5 | |
| I-95 | B | | | | 2276.1 | 2277.5 | 2275.6 | |
| I-96 | C | | | | 2276.1 | 1140.5 | 1138.6 | |
| I-97 | A | B | | | 2149.0 | 2150.5 | 2148.6 | |
| I-98 | A | C | | | 2149.0 | 1075.8 | 1073.9 | |
| I-99 | A | D | + | | 2179.0 | 1090.8 | 2178.4 | |
| I-100 | A | | | | 2179.0 | 1090.8 | 1088.9 | |
| I-101 | A | B | | | 2205.0 | 1103.8 | 2204.4 | |
| I-102 | A | C | | | 2205.0 | 1103.8 | 2204.4 | |
| I-103 | A | C | | | 2231.0 | 1116.8 | 2230.5 | |
| I-104 | A | B | | | 2165.0 | 1083.8 | 2164.4 | |
| I-105 | A | | | | 2165.0 | 1083.8 | 1081.9 | |
| I-106 | A | B | + | | 2191.0 | 1096.8 | 2190.4 | |
| I-107 | A | | | | 2191.0 | 1097.0 | 1095.0 | |
| I-108 | A | D | | | 2221.0 | 1111.8 | 2220.5 | |
| I-109 | B | D | ++ | | 2221.0 | 1111.8 | 2220.5 | |
| I-110 | B | C | | | 2247.1 | 1124.8 | 2246.5 | |
| I-111 | B | C | | | 2247.1 | 1224.8 | 2246.5 | |
| I-112 | A | C | | | 2157.9 | 1080.3 | 1078.3 | |
| I-113 | A | D | | | 2157.0 | 1079.1 | 2156.3 | |
| I-114 | B | | | | 2157.0 | 1079.8 | 2156.3 | |
| I-115 | E | | | | 2144.0 | 1073.3 | 2143.3 | |
| I-116 | E | | | | 2144.0 | 1073.3 | 2143.3 | |
| I-117 | C | | | | 2157.0 | 2158.2 | 2156.3 | |
| I-118 | E | | | | 2144.0 | 1073.3 | 2143.3 | |
| I-119 | E | | | | 2144.0 | 1073.3 | 2143.3 | |
| I-120 | A | B | | | 2172.0 | 2173.2 | 2171.3 | |
| I-121 | A | B | ++ | | 2172.0 | 2173.2 | 2171.3 | |
| I-122 | A | D | | | 2200.0 | 2201.2 | 2199.3 | |
| I-123 | B | D | | | 2234.0 | 1118.3 | 2233.4 | |
| I-124 | A | D | | | 2186.0 | 1094.3 | 1092.3 | |
| I-125 | A | D | ++ | | 2186.0 | 1094.3 | 1092.3 | |
| I-126 | A | C | ++ | | 2172.0 | 1087.2 | 2171.3 | |
| I-127 | A | C | ++ | | 2172.0 | 1087.2 | 2171.3 | |
| I-128 | B | C | ++ | | 2200.0 | 2201.2 | 2199.4 | |
| I-129 | A | C | ++ | | 2186.0 | 2187.2 | 2185.3 | |
| I-130 | B | D | | | 2302.1 | 1152.3 | — | |
| I-131 | C | D | | | 2302.1 | 2303.4 | — | |
| I-132 | B | D | | | 2302.1 | 2303.5 | — | |
| I-133 | D | D | | | 2306.1 | 2307.5 | — | A |
| I-134 | C | D | | | 2306.1 | 2303.3 | — | A |
| I-135 | E | D | | | 2306.1 | 2307.6 | — | A |
| I-136 | A | B | | | 2252.0 | 1127.6 | 1125.6 | |
| I-137 | A | B | | | 2294.1 | 1148.6 | 1146.6 | |
| I-138 | A | B | + | + | 2266.0 | 1134.6 | 1132.6 | |
| I-139 | A | B | + | | 2264.0 | 1133.6 | 1131.6 | |
| I-140 | A | A | + | | 2278.0 | 1140.6 | 1138.6 | |
| I-141 | A | B | + | | 2292.0 | 1147.6 | 1145.5 | |
| I-142 | C | C | +++ | | 2251.0 | 1127.0 | 1125.0 | |
| I-143 | C | D | | | 2293.1 | 1148.1 | 1146.1 | |
| I-144 | A | C | ++ | | 2265.0 | 1134.1 | 1132.3 | |
| I-145 | E | | | | 2238.0 | 1120.5 | 1118.6 | |
| I-146 | E | | | | 2280.1 | 2282.1 | 2280.2 | |
| I-147 | D | | | | 2252.1 | 1127.6 | 1125.6 | |
| I-148 | C | | | | 1976.0 | 989.2 | 987.0 | |
| I-149 | C | D | | | 2045.0 | 2046.7 | 2044.6 | |
| I-150 | E | | | | 2018.0 | 2019.4 | 2017.4 | |
| I-151 | B | D | | | 1981.9 | 993.3. | 991.2. | |
| I-152 | D | | | | 2051.0 | 2011.4 | 2009.4 | |
| I-153 | E | | | | 1980.0 | 991.3. | 989.2. | A |
| I-154 | E | | | | 2049.1 | 2050.6 | 2048.4 | A |
| I-155 | E | | | | 1953.0 | 977.7 | 975.5 | A |
| I-156 | E | | | | 2022.1 | 2023.6 | 2021.5 | A |
| I-157 | E | | | | 1985.9 | 994.2. | 1985.3 | A |
| I-158 | E | | | | 2055.0 | 2056.6 | 2054.5 | A |
| I-159 | A | B | | | 2073.9 | 1038.3 | 1036.2 | |
| I-160 | A | C | | | 2046.9 | 1024.8 | 1022.9 | |
| I-161 | C | | | | 2060.9 | 1031.8 | 1029.6 | |
| I-162 | C | D | | | 2060.9 | 2062.0 | 2060.1 | |
| I-163 | A | C | +++ | | 2060.9 | 1031.7 | 2060.1 | |
| I-164 | B | D | | | 2088.0 | 1045.2 | 2087.2 | |
| I-165 | A | C | | | 2073.9 | 1038.2 | 2073.2 | |
| I-166 | B | C | | | 2046.9 | 1024.7 | 2046.1 | |
| I-167 | E | | | | 2060.9 | 1031.7 | 2060.2 | |
| I-168 | C | D | | | 2060.9 | 1031.7 | 2060.2 | |
| I-169 | B | D | | | 2060.9 | 1031.7 | 2060.2 | |
| I-170 | B | D | | | 2088.0 | 1045.2 | 2087.2 | |
| I-171 | B | D | | | 2078.0 | 1040.2 | 2077.2 | A |
| I-172 | D | | | | 2051.0 | 1026.7 | 2050.3 | A |
| I-173 | E | | | | 2065.0 | 1033.7 | 2064.2 | A |
| I-174 | E | | | | 2065.0 | 1033.7 | 2064.2 | A |
| I-175 | E | | | | 2065.0 | 1033.7 | 2064.2 | A |
| I-176 | E | | | | 2092.0 | 1047.2 | 2091.2 | A |
| I-177 | C | D | | | 2078.0 | 1040.2 | 2077.2 | A |
| I-178 | E | | | | 2051.0 | 1026.7 | 2050.2 | A |
| I-179 | E | | | | 2065.0 | 1033.7 | 2064.2 | A |
| I-180 | E | | | | 2065.0 | 1033.7 | 2064.2 | A |
| I-181 | E | | | | 2065.0 | 1033.7 | 2064.2 | A |
| I-182 | E | | | | 2092.0 | 1047.2 | 1887.9 | A |
| I-183 | A | C | ++ | | 2074.9 | 1038.7 | 1036.8 | |
| I-184 | A | | | | 2137.0 | 1069.7 | 1067.7 | |
| I-185 | A | | | | 2074.9 | 1038.7 | 1036.8 | |
| I-186 | A | | | | 2137.0 | 1069.7 | 1067.6 | |
| I-187 | A | C | +++ | | 2117.0 | 1059.7 | 1057.6 | |
| I-188 | A | C | | | 2179.0 | 1090.7 | 1088.8 | |
| I-189 | A | C | | | 2179.0 | 1090.7 | 1088.7 | |
| I-190 | A | | | | 2103.0 | 1052.7 | 1050.8 | |
| I-191 | B | C | | | 2165.0 | 1083.7 | 1081.7 | |
| I-192 | A | | | | 2165.0 | 1083.7 | 1081.7 | |
| I-193 | A | C | | | 2152.9 | 2175.8 | 2152.0 | |
| I-194 | A | A | | | 2152.9 | 2175.8 | 2152.0 | |
| I-195 | A | | | | 2184.0 | 2184.9 | 2183.1 | |
| I-196 | B | C | | | 2137.0 | 1069.6 | 2136.1 | |
| I-197 | A | B | | | 2137.0 | 2159.8 | 2136.0 | |
| I-198 | A | C | | | 2074.9 | 2097.8 | 2073.9 | |
| I-199 | A | C | | | 2060.9 | 2083.8 | 2060.0 | |
| I-200 | B | D | | | 2046.9 | 2069.8 | 2046.1 | |
| I-201 | B | C | | | 2074.0 | 2096.8 | 2073.0 | |
| I-202 | A | D | | | 2083.0 | 2084.1 | 2082.0 | |
| I-203 | E | D | | | 2109.0 | 2131.9 | 2108.1 | |
| I-204 | A | C | | | 2204.9 | 1103.6 | 1101.7 | |
| I-205 | A | C | | | 2204.9 | 2227.8 | 2204.0 | |
| I-206 | A | | | | 2204.9 | 2227.8 | 2204.0 | |
| I-207 | A | B | | | 2204.9 | 2227.8 | 2204.0 | |
| I-208 | A | C | | | 2154.9 | 2177.8 | 2154.0 | |
| I-209 | E | D | | | 2045.0 | 2067.8 | 2044.0 | |
| I-210 | B | C | | | 2032.9 | 2055.8 | 2032.0 | |
| I-211 | C | D | | | 2132.0 | 2154.9 | 2131.1 | |
| I-212 | A | C | | | 2059.9 | 2082.8 | 2059.0 | |
| I-213 | B | C | | | 2016.9 | 2039.8 | 2015.9 | |
| I-214 | B | D | | | 2102.0 | 2103.2 | 2101.1 | |
| I-215 | A | | | | 2074.9 | 1038.7 | 1036.7 | |
| I-216 | B | C | | | 2046.9 | 1024.7 | 1022.6 | |
| I-217 | E | D | | | 2123.0 | 1062.7 | 1060.8 | |
| I-218 | B | C | | | 2136.0 | 1069.2 | 1067.2 | |
| I-219 | C | | | | 2059.0 | 1030.7 | 1028.7 | |
| I-220 | C | D | | | 2093.0 | 1047.7 | 1045.5 | |
| I-221 | C | D | | | 2074.0 | 2075.2 | 2073.2 | |
| I-222 | E | D | | | 2059.0 | 1030.7 | 1028.6 | |
| I-223 | A | B | | | 2211.0 | 2212.2 | 2210.2 | |
| I-224 | A | D | | | 2324.1 | 2325.3 | 2323.2 | |
| I-225 | A | | | | 2308.1 | 2309.3 | 2307.2 | |
| I-226 | A | B | | | 2165.0 | 1083.7 | 1081.8 | |
| I-227 | A | B | | | 2252.0 | 2253.1 | 2251.2 | |
| I-228 | A | C | | | 2278.1 | 2279.3 | 2277.3 | |
| I-229 | A | D | | | 2365.1 | 2366.4 | 2364.3 | |
| I-230 | A | | | | 2224.0 | 1113.2 | 1111.2 | |
| I-231 | A | D | | | 2250.0 | 2251.1 | 2049.2 | |
| I-232 | A | D | | | 2337.1 | 2338.1 | 2336.2 | |
| I-233 | A | D | | | 2010.9 | 1006.6 | 2009.9 | A |
| I-234 | A | D | | | 2024.9 | 1013.6 | 2023.9 | A |
| I-235 | A | D | | | 2038.9 | 1020.6 | 2038.1 | A |
| I-236 | A | D | | | 2010.9 | 1006.6 | 2009.9 | A |
| I-237 | A | D | | +++ | 1996.9 | 999.6. | 1995.8 | A |
| I-238 | A | D | | | 2010.9 | 1006.6 | 2009.9 | A |
| I-239 | C | D | | | 2045.9 | 2046.8 | 2044.8 | A |
| I-240 | B | D | | | 2059.9 | 1031.1 | 2058.9 | A |
| I-241 | C | D | | | 2073.9 | 1038.1 | 2073.2 | A |
| I-242 | C | D | | | 2045.9 | 1024.1 | 2044.8 | A |
| I-243 | B | D | | | 2031.9 | 1017.1 | 2030.8 | A |
| I-244 | B | D | | | 2045.9 | 1024.1 | 2045.2 | A |
| I-245 | A | B | | + | 2060.9 | 1031.6 | 2059.9 | |
| I-246 | A | B | | | 2060.9 | 1031.6 | 2060.2 | |
| I-247 | A | B | | | 2060.9 | 1031.6 | 2060.2 | |
| I-248 | A | B | | | 2046.9 | 1024.6 | 2045.9 | |
| I-249 | A | C | | | 2094.9 | 1048.6 | 2093.9 | |
| I-250 | A | C | | | 2100.9 | 1051.6 | 2100.2 | |
| I-251 | A | B | | | 2086.9 | 1044.6 | 2085.9 | |
| I-252 | A | C | | | 2058.9 | 1030.6 | 2057.9 | |
| I-253 | A | A | | + | 2086.9 | 1044.6 | 2086.2 | |
| I-254 | A | C | | | 2184.8 | 1093.5 | 2183.9 | |
| I-255 | A | C | | | 2074.9 | 1038.6 | 2073.9 | |
| I-256 | A | C | | | 2086.9 | 1044.6 | 2086.2 | |
| I-257 | A | C | | | 2149.0 | 1075.6 | 2147.9 | |
| I-258 | A | C | | | 2086.9 | 1044.6 | 2085.9 | |
| I-259 | B | C | | | 2149.0 | 1075.6 | 2147.9 | |
| I-260 | A | | | | 2100.9 | 1082.5 | 2161.9 | |
| I-261 | A | | | | 2163.0 | 1051.6 | 2100.2 | |
| I-262 | A | B | + | + | 2114.9 | 1058.6 | 2114.2 | |
| I-263 | A | C | | | 2114.9 | 1058.6 | 2113.9 | |
| I-264 | A | | | | 2100.9 | 1051.6 | 2099.9 | |
| I-265 | A | C | | | 2100.9 | 1051.6 | 2099.9 | |
| I-266 | C | D | | | 1992.9 | 1996.6 | 1994.8 | |
| I-267 | C | | | | 1992.9 | 1993.9 | 1992.0 | |
| I-268 | B | D | | | 2006.9 | 1005.7 | 2008.3 | |
| I-269 | B | D | | | 2006.9 | 1004.6 | 1002.6 | |
| I-270 | A | C | | | 2021.0 | 1011.6 | 1009.7 | |
| I-271 | A | C | | | 2035.0 | 1019.8 | 2036.6 | |
| I-272 | A | B | | | 2035.0 | 1018.6 | 1016.8 | |
| I-273 | B | D | | | 2035.0 | 1019.8 | 2036.8 | |
| I-274 | B | D | | | 2035.0 | 1018.6 | 1016.6 | |
| I-275 | A | C | | | 2049.0 | 1026.7 | 2050.4 | |
| I-276 | A | C | | | 2049.0 | 1025.6 | 1023.7 | |
| I-277 | B | D | | | 2049.0 | 1026.8 | 2050.4 | |
| I-278 | B | D | | | 2049.0 | 1025.7 | 1023.8 | |
| I-279 | A | C | | | 2069.0 | 1036.8 | 2070.8 | |
| I-280 | A | B | | | 2069.0 | 2070.1 | 2068.2 | |
| I-281 | A | C | | | 2033.0 | 1018.7 | 2034.4 | |
| I-282 | A | C | | | 2033.0 | 1017.6 | 1015.7 | |
| I-283 | A | C | | | 2047.0 | 1024.6 | 1022.7 | |
| I-284 | A | C | | | 2061.0 | 1031.6 | 1029.4 | |
| I-285 | A | C | | | 2075.0 | 1038.6 | 1036.7 | |
| I-286 | A | | | | 2151.0 | 2152.1 | 2150.2 | |
| I-287 | B | D | | | 2151.0 | 1076.7 | 2150.0 | |
| I-288 | A | B | | | 2151.0 | 2151.9 | 2150.0 | |
| I-289 | A | C | | | 2151.0 | 1076.6 | 1074.6 | |
| I-290 | B | | | | 2151.0 | 1076.7 | 2150.3 | |
| I-291 | E | D | ++ | | 2165.0 | 1083.7 | 2164.0 | |
| I-292 | A | C | | | 2058.9 | 2060.0 | 2058.0 | |
| I-293 | A | B | | | 2099.0 | 2099.9 | 2098.1 | |
| I-294 | A | A | | + | 2083.0 | 2083.9 | 2082.1 | |
| I-295 | A | A | | | 2146.9 | 1075.0 | 1073.1 | |
| I-296 | A | B | | | 2102.9 | 1052.8 | 1050.9 | |
| I-297 | A | B | | | 2094.0 | 2094.9 | 2093.1 | |
| I-298 | A | A | | | 2113.9 | 2114.9 | 2113.0 | |
| I-299 | A | B | | ++ | 2070.0 | 2071.2 | 2069.1 | |
| I-300 | A | C | | +++ | 2070.0 | 2071.2 | 2069.0 | |
| I-301 | A | D | | +++ | 2070.0 | 2071.1 | 2069.1 | |
| I-302 | A | D | | | 2059.0 | 2060.0 | 2058.0 | |
| I-303 | A | D | | | 2843.3 | 1422.9 | 1420.8 | |
| I-304 | A | C | | + | 2018.9 | 1010.6 | 2017.9 | |
| I-305 | A | C | | | 2110.9 | 1056.6 | 2110.0 | |
| I-306 | A | D | | | 2133.9 | 1068.1 | 2133.0 | |
| I-307 | A | D | | | 2034.9 | 1018.6 | 2033.9 | |
| I-308 | A | | | | 2032.9 | 2034.1 | 2032.1 | |
| I-309 | A | | | | 2080.9 | 2082.1 | 2080.1 | |
| I-310 | A | B | | | 2170.9 | 1086.6 | 2170.1 | |
| I-311 | A | B | | | 2117.0 | 1059.6 | 2116.0 | |
| I-312 | A | B | | | 2072.9 | 1037.6 | 2072.0 | |
| I-313 | A | D | | +++ | 2131.9 | 2133.1 | 2131.0 | |
| I-314 | A | C | | | 2165.9 | 2167.2 | 2165.3 | |
| I-315 | A | C | | | 2121.0 | 2122.1 | 2120.3 | |
| I-316 | B | D | | | 2094.0 | 2095.2 | 2093.3 | |
| I-317 | E | | | | 2108.0 | 2109.1 | 2107.3 | |
| I-318 | A | C | | | 2121.0 | 2122.1 | 2120.3 | |
| I-319 | A | D | | | 2234.0 | 2235.3 | 2233.4 | |
| I-320 | B | D | | | 2207.0 | 2208.3 | 2206.4 | |
| I-321 | E | | | | 2221.0 | 2222.2 | 2220.3 | |
| I-322 | A | C | | | 2234.0 | 2235.3 | 2233.4 | |
| I-323 | A | B | | | 2187.0 | 2188.2 | 2186.1 | |
| I-324 | A | D | | | 2160.0 | 2161.1 | 2159.3 | |
| I-325 | C | | | | 2174.0 | 2175.1 | 2173.2 | |
| I-326 | A | C | | | 2187.0 | 2188.2 | 2186.3 | |
| I-327 | A | | | | 2148.9 | 2150.6 | 2148.7 | |
| I-328 | A | B | | | 2086.9 | 2088.6 | 2086.8 | |
| I-329 | A | A | | | 2080.9 | 2082.6 | 2080.8 | |
| I-330 | A | C | | | 2109.0 | 1055.6 | 1053.6 | |
| I-331 | A | | | | 2158.9 | 2160.8 | 2158.9 | |
| I-332 | A | C | | | 2096.9 | 2098.7 | 2096.9 | |
| I-333 | A | | | | 2090.9 | 1047.0 | 1044.9 | |
| I-334 | A | B | | | 2119.0 | 1060.6 | 1058.6 | |
| I-335 | A | A | + | + | 2084.9 | 2085.9 | 2084.0 | |
| I-336 | A | B | ++ | | 2084.9 | 1043.6 | 2084.1 | |
| I-337 | A | B | + | | 2070.9 | 2071.9 | 2070.0 | |
| I-338 | A | C | + | | 2070.9 | 1036.6 | 2070.0 | |
| I-339 | C | D | | | 2249.8 | 2250.7 | 2248.9 | |
| I-340 | C | | | | 2397.8 | 2398.7 | 2396.8 | |
| I-341 | C | D | | | 2397.8 | 2398.8 | 2396.9 | |
| I-342 | A | B | + | | 2204.0 | 1103.1 | 1101.2 | |
| I-343 | A | B | | | 2275.0 | 1138.6 | 1136.5 | |
| I-344 | A | D | | | 2113.0 | 2113.9 | 2112.1 | |
| I-345 | A | C | | | 2118.9 | 2119.9 | 2118.1 | |
| I-346 | B | | | | 2156.9 | 2157.9 | 2156.0 | |
| I-347 | B | D | | | 2357.0 | 1179.6 | 1177.6 | |
| I-348 | A | | | | 2204.9 | 2206.0 | 2204.1 | |
| I-349 | A | D | | | 2113.9 | 2114.8 | 2113.0 | |
| I-350 | A | D | | | 2119.9 | 2120.9 | 2119.0 | |
| I-351 | B | D | | | 2157.9 | 2158.8 | 2156.9 | |
| I-352 | B | D | | | 2358.0 | 2358.9 | 2357.0 | |
| I-353 | A | | | | 2052.9 | 1027.6 | 1025.6 | |
| I-354 | B | D | | | 2067.0 | 2067.9 | 2066.1 | |
| I-355 | A | C | | | 2130.9 | 1066.6 | 1064.7 | |
| I-356 | A | D | | | 2124.9 | 2125.9 | 2124.1 | |
| I-357 | A | C | | | 2124.9 | 1063.7 | 1061.9 | |
| I-358 | A | B | | | 2090.9 | 2091.9 | 2090.1 | |
| I-359 | A | C | | | 2113.9 | 2114.9 | 2113.0 | |
| I-360 | D | | | | 2040.9 | 2041.9 | 2040.1 | |
| I-361 | E | | | | 2040.9 | 1021.6 | 1019.8 | |
| I-362 | A | | | | 2040.9 | 1021.6 | 1019.5 | |
| I-363 | B | D | | | 2014.9 | 2015.9 | 2014.0 | |
| I-364 | A | A | | | 2080.9 | 2082.0 | 2080.3 | |
| I-365 | A | B | | | 2080.9 | 2081.9 | 2080.0 | |
| I-366 | E | | | | 2067.0 | 1034.6 | 1032.7 | |
| I-367 | B | | | | 2028.9 | 2029.9 | 2028.0 | |
| I-368 | C | | | | 2065.9 | 2067.0 | 2065.1 | |
| I-369 | A | C | | | 2142.9 | 1072.6 | 1070.6 | |
| I-370 | E | | | | 2028.9 | 2029.9 | 2028.0 | |
| I-371 | C | | | | 2064.9 | 2066.1 | 2064.0 | |
| I-372 | E | | | | 2026.9 | 2027.9 | 2026.0 | |
| I-373 | E | | | | 2041.9 | 2042.9 | 2041.0 | |
| I-374 | C | | | | 2055.9 | 2056.9 | 2055.1 | |
| I-375 | A | C | | + | 2205.9 | 1104.2 | 1102.2 | |
| I-376 | C | D | | | 2205.9 | 1104.2 | 1102.1 | |
| I-377 | B | C | | | 2205.9 | 1104.1 | 1102.1 | |
| I-378 | A | B | | | 2188.0 | 1095.1 | 1093.2 | |
| I-379 | A | B | | | 2146.0 | 1074.1 | 1072.3 | |
| I-380 | A | D | | | 2152.9 | 1077.6 | 1075.7 | |
| I-381 | A | B | | | 2160.0 | 1081.1 | 1079.3 | |
| I-382 | A | B | | | 2100.9 | 1051.6 | 1050.0 | |
| I-383 | A | A | | | 2100.9 | 1051.6 | 1049.6 | |
| I-384 | A | | | | 2115.9 | 2117.2 | 2115.1 | |
| I-385 | A | D | | | 2115.9 | 2117.1 | 2115.1 | |
| I-386 | A | A | | +++ | 2136.9 | 1069.6 | 1067.6 | |
| I-387 | A | C | | | 2126.9 | 2128.1 | 2126.1 | |
| I-388 | A | A | | | 2088.9 | 1045.6 | 1043.6 | |
| I-389 | A | B | | | 2076.9 | 1039.6 | 1037.7 | |
| I-390 | A | B | | | 2076.9 | 1039.6 | 1037.7 | |
| I-391 | A | C | | | 2117.9 | 1060.1 | 1058.2 | |
| I-392 | A | C | | | 2117.9 | 1060.1 | 1058.2 | |
| I-393 | A | C | | | 2103.9 | 1053.1 | 1051.2 | |
| I-394 | A | | | | 2103.9 | 2104.7 | 2102.9 | |
| I-395 | E | | | | 2024.9 | 2025.9 | 2024.1 | |
| I-396 | A | B | | | 2103.0 | 1052.6 | 2102.2 | |
| I-397 | A | B | | | 2088.9 | 2111.9 | 2088.1 | |
| I-398 | A | B | | | 2104.9 | 2106.0 | 2104.1 | |
| I-399 | A | A | | | 2190.0 | 1096.0 | 2189.0 | |
| I-400 | A | B | | | 2176.0 | 1089.0 | 2175.0 | |
| I-401 | A | A | | | 2192.0 | 2192.8 | 2191.0 | |
| I-402 | A | B | | | 2174.0 | 2197.0 | 2173.2 | |
| I-403 | A | | | | 2174.0 | 1088.1 | 2173.0 | |
| I-404 | A | A | | | 2160.0 | 2182.8 | 2159.0 | |
| I-405 | A | A | | | 2160.0 | 2182.8 | 2159.0 | |
| I-406 | A | A | | | 2176.0 | 1089.1 | 2075.1 | |
| I-407 | A | B | | | 2188.0 | 2210.9 | 2187.0 | |
| I-408 | A | A | | | 2188.0 | 2210.8 | 2187.0 | |
| I-409 | A | A | | | 2174.0 | 2196.8 | 2173.0 | |
| I-410 | A | | | | 2174.0 | 2197.1 | 2173.3 | |
| I-411 | A | B | | | 2190.0 | 2190.8 | 2188.8 | |
| I-412 | A | A | | | 2190.0 | 2213.1 | 2189.3 | |
| I-413 | A | A | | | 2112.9 | 1057.6 | 2112.0 | |
| I-414 | A | A | | | 2127.0 | 2127.8 | 2126.0 | |
| I-415 | A | A | | | 2114.9 | 2115.8 | 2113.9 | |
| I-416 | A | A | | | 2171.9 | 2172.8 | 2171.0 | |
| I-417 | A | B | | | 2200.0 | 1101.1 | 2199.0 | |
| I-418 | A | B | | | 2079.0 | 2079.8 | 2077.9 | |
| I-419 | A | | | | 2093.0 | 2094.1 | 2092.1 | |
| I-420 | A | B | | | 2093.0 | 2093.8 | 2091.9 | |
| I-421 | A | D | | | 2070.9 | 2071.9 | 2070.0 | |
| I-422 | A | D | | | 2064.9 | 2065.8 | 2063.9 | |
| I-423 | A | C | | | 2084.9 | 2085.8 | 2083.9 | |
| I-424 | A | C | | | 2079.0 | 2079.9 | 2078.0 | |
| I-425 | B | D | | | 2008.9 | 1005.5 | 1003.6 | A |
| I-426 | A | D | | | 2022.9 | 2024.0 | 2022.1 | A |
| I-427 | A | D | | | 2004.8 | 1003.5 | 1001.6 | |
| I-428 | A | D | | | 2006.9 | 2007.8 | 2006.0 | B |
| I-429 | A | D | | | 2018.9 | 1010.5 | 1008.7 | |
| I-430 | B | D | | | 2020.9 | 2022.3 | 2020.4 | |
| I-431 | A | D | | | 2020.9 | 2022.1 | 2020.2 | B |
| I-432 | A | D | | | 2117.0 | 2118.8 | 2116.9 | |
| I-433 | A | C | | | 2117.0 | 1059.8 | 1057.9 | |
| I-434 | A | D | | | 2103.0 | 1052.7 | 1050.9 | |
| I-435 | A | D | | | 2090.9 | 1046.7 | 1044.8 | |
| I-436 | A | C | | | 2090.9 | 2092.0 | 2090.2 | |
| I-437 | A | D | | | 2090.9 | 1046.7 | 1044.9 | |
| I-438 | A | D | | | 2151.0 | 1076.7 | 1074.9 | |
| I-439 | A | D | | | 2088.9 | 1045.8 | 1043.9 | |
| I-440 | A | D | | | 2086.9 | 1044.7 | 1042.8 | |
| I-441 | A | D | | | 2074.9 | 1038.7 | 1036.9 | |
| I-442 | B | D | | | 2074.9 | 1038.7 | 1036.7 | |
| I-443 | A | D | | | 2072.9 | 1037.7 | 1035.8 | |
| I-444 | A | C | | | 2090.9 | 1046.9 | 1044.9 | |
| I-445 | A | C | | | 2104.9 | 1053.7 | 1051.8 | |
| I-446 | A | D | | | 2104.9 | 2106.3 | 2104.5 | |
| I-447 | A | D | | | 2103.0 | 1052.8 | 1050.8 | |
| I-448 | A | D | | | 2103.0 | 1052.8 | 1051.0 | |
| I-449 | A | D | | | 2117.0 | 1059.8 | 1057.9 | |
| I-450 | A | D | | | 2117.0 | 1059.8 | 1057.9 | |
| I-451 | A | D | | | 2131.9 | 1067.2 | 1065.4 | |
| I-452 | A | D | | | 2131.9 | 1067.2 | 1065.3 | |
| I-453 | A | D | | | 2202.0 | 1102.3 | 1100.4 | |
| I-454 | A | D | | | 2132.0 | 2133.5 | 2131.5 | |
| I-455 | A | D | | | 2132.0 | 2133.6 | 2131.5 | |
| I-456 | A | D | | | 2151.0 | 1076.8 | 1074.8 | |
| I-457 | A | C | | | 2151.0 | 1076.7 | 1074.8 | |
| I-458 | A | B | | | 2074.9 | 1038.7 | 2074.3 | |
| I-459 | A | | | | 2074.9 | 2098.0 | 2074.2 | |
| I-460 | A | | | | 2162.0 | 2185.1 | 2161.3 | |
| I-461 | A | C | | | 2162.0 | 1082.3 | 2161.5 | |
| I-462 | A | C | | | 2117.0 | 2140.2 | 2116.3 | |
| I-463 | A | | | | 2204.0 | 2227.1 | 2203.4 | |
| I-464 | A | B | | | 2275.0 | 2298.3 | 2274.7 | |
| I-465 | A | B | | | 2317.1 | 2340.3 | 2316.5 | |
| I-466 | A | | | | 2317.1 | 2340.3 | 2316.5 | |
| I-467 | A | B | | | 2317.1 | 1159.8 | 2316.6 | |
| I-468 | A | C | | | 2317.1 | 1095.3 | 1093.4 | |
| I-469 | B | D | | | 2155.9 | 1070.0 | 1068.1 | |
| I-470 | E | D | | | 2103.0 | 1052.8 | 1050.8 | |
| I-471 | A | D | | | 2082.9 | 2083.7 | 2081.8 | |
| I-472 | A | B | | | 2114.9 | 1059.1 | 2115.3 | |
| I-473 | A | C | | | 2046.9 | 2048.8 | 2047.1 | |
| I-474 | A | C | | | 2046.9 | 1025.1 | 1023.3 | |
| I-475 | A | C | | | 2072.9 | 1037.9 | 2073.1 | |
| I-476 | A | C | | | 2072.9 | 1038.1 | 2073.2 | |
| I-477 | A | C | | | 2096.9 | 2098.7 | 2096.9 | |
| I-478 | A | B | | | 2086.9 | 2088.8 | 2086.8 | |
| I-479 | A | C | | | 2046.9 | 1024.9 | 2047.6 | |
| I-480 | A | D | | | 2046.9 | 1025.1 | 1023.1 | |
| I-481 | A | | | | 2130.9 | 1067.1 | 2131.1 | |
| I-482 | A | | | | 2130.9 | 1067.1 | 1065.1 | |
| I-483 | A | C | | | 2063.0 | 2064.2 | 2062.2 | |
| I-484 | A | D | | | 2063.0 | 2064.2 | 2062.2 | |
| I-485 | A | C | | | 2069.0 | 2070.2 | 2068.1 | |
| I-486 | A | | | | 2069.0 | 2070.2 | 2068.1 | |
| I-487 | A | C | | | 2097.0 | 1049.8 | 2096.6 | |
| I-488 | A | | | | 2097.0 | 1049.8 | 2096.7 | |
| I-489 | A | | | | 2029.0 | 2030.2 | 2028.2 | |
| I-490 | B | | | | 2029.0 | 1015.6 | 2028.2 | |
| I-491 | B | | | | 2055.0 | 1028.6 | 2054.2 | |
| I-492 | B | | | | 2055.0 | 1028.6 | 2054.2 | |
| I-493 | A | C | | | 2303.0 | 1152.9 | 1151.1 | |
| I-494 | A | C | | | 2317.0 | 1159.9 | 1157.9 | |
| I-495 | A | B | | | 2317.0 | 1159.9 | 1158.1 | |
| I-496 | A | C | | | 2275.0 | 1138.9 | 1137.1 | |
| I-497 | A | B | | | 2275.0 | 1138.9 | 1137.1 | |
| I-498 | A | B | | | 2289.1 | 1145.9 | 1144.1 | |
| I-499 | A | C | | | 2289.1 | 1145.9 | 1143.9 | |
| I-500 | A | B | | | 2289.1 | 1145.9 | 1144.1 | |
| I-501 | A | C | | | 2289.1 | 1145.9 | 2287.9 | |
| I-502 | A | C | | | 2345.1 | 1173.9 | 1172.1 | |
| I-503 | A | C | | | 2345.1 | 1174.1 | 1171.9 | |
| I-504 | A | C | | | 2359.1 | 1180.9 | 1179.1 | |
| I-505 | A | C | | | 2359.1 | 1181.1 | 1178.9 | |
| I-506 | A | C | | | 2289.0 | 1145.9 | 1144.1 | |
| I-507 | A | B | | | 2303.0 | 1152.9 | 1151.1 | |
| I-508 | A | B | | | 2275.0 | 1138.9 | 1137.1 | |
| I-509 | A | B | | | 2275.0 | 1138.9 | 1137.1 | |
| I-510 | A | B | | | 2275.0 | 1138.9 | 1136.9 | |
| I-511 | A | B | | | 2275.0 | 1139.1 | 1137.1 | |
| I-512 | A | C | | | 2331.1 | 1166.9 | 1165.1 | |
| I-513 | A | C | | | 2331.1 | 1166.9 | 1164.9 | |
| I-514 | A | C | | | 2132.9 | 2133.5 | 2131.7 | |
| I-515 | A | D | | | 2146.9 | 1074.4 | 1072.5 | |
| I-516 | A | C | | | 2161.0 | 1081.4 | 1079.4 | |
| I-517 | A | D | | | 2781.3 | 1392.1 | 1390.2 | |
| I-518 | E | | | | 2809.3 | 1406.2 | 1404.2 | |
| I-519 | A | D | | | 2137.9 | 2139.3 | 2137.4 | |
| I-520 | A | D | | | 2151.9 | 2153.3 | 2151.4 | |
| I-521 | A | | | | 2118.9 | 2121.1 | 2119.0 | |
| I-522 | C | | | | 2104.9 | 2107.1 | 2105.1 | |
| I-523 | A | | | | 2132.9 | 2135.1 | 2133.1 | |
| I-524 | B | | | | 2118.9 | 2121.1 | 2118.9 | |
| I-525 | A | | | | 2146.9 | 2149.2 | 2146.9 | |
| I-526 | C | | | | 2133.0 | 2135.1 | 2133.2 | |
| I-527 | A | | | | 2117.0 | 2119.2 | 2117.1 | |
| I-528 | A | | | | 2117.0 | 2119.0 | 2117.0 | |
| I-529 | A | | | | 2151.0 | 2153.1 | 2150.8 | |
| I-530 | A | | | | 2165.0 | 2167.2 | 2165.1 | |
| I-531 | A | | | | 2129.0 | 2131.2 | 2129.1 | |
| I-532 | A | | | | 2129.0 | 2131.0 | 2129.1 | |
| I-533 | A | | | | 2181.0 | 2183.1 | 2180.6 | |
| I-534 | A | | | | 2174.0 | 2176.0 | 2174.0 | |
| I-535 | B | | | | 2021.0 | 2022.3 | 2020.5 | |
| I-536 | B | | | | 2035.0 | 2036.5 | 2034.6 | |
| I-537 | A | | | | 2061.0 | 1031.8 | 1029.8 | |
| I-538 | A | | | | 2049.0 | 2050.4 | 2048.5 | |
| I-539 | A | | | | 2049.0 | 2050.4 | 2048.6 | |
| I-540 | C | | | | 2091.0 | 2092.5 | 2090.6 | |
| I-541 | A | | | | 2074.9 | 2076.4 | 2074.6 | |
| I-542 | A | | | | 2112.0 | 2113.3 | 2111.5 | |
| I-543 | B | | | | 2088.9 | 1045.8 | 1043.9 | |
| I-544 | C | | | | 2088.9 | 1045.8 | 1043.8 | |
| I-545 | C | D | | | 2103.0 | 2104.5 | 2102.5 | |
| I-546 | C | | | | 2103.0 | 1052.8 | 1050.9 | |
| I-547 | A | D | | | 2088.9 | 2090.4 | 2088.2 | |
| I-548 | C | D | | | 2103.0 | 2104.4 | 2102.5 | |
| I-549 | E | D | | | 2117.0 | 2118.4 | 2116.6 | |
| I-550 | E | D | | | 2117.0 | 2118.4 | 2116.6 | |
| I-551 | E | D | | | 2131.0 | 2132.5 | 2130.6 | |
| I-552 | E | | | | 2040.9 | 2042.4 | 2040.5 | |
| I-553 | E | D | | | 2055.0 | 2056.6 | 2054.7 | |
| I-554 | E | D | | | 2026.9 | 2028.4 | 2026.6 | |
| I-555 | E | D | | | 2040.9 | 2042.3 | 2040.5 | |
| I-556 | E | D | | | 2069.0 | 2070.5 | 2068.6 | |
| I-557 | A | | | | 2074.9 | 2077.1 | 2075.0 | |
| I-558 | A | | | | 2060.9 | 2063.0 | 2060.9 | |
| I-559 | A | | | | 2046.9 | 2049.0 | 2047.2 | |
| I-560 | A | | | | 2074.9 | 2077.1 | 2074.8 | |
| I-561 | A | | | | 2088.9 | 2091.1 | 2089.1 | |
| I-562 | B | D | | | 2127.9 | 2129.1 | 2127.2 | A |
| I-563 | A | D | | | 2141.9 | 2143.0 | 2141.2 | A |
| I-564 | B | D | | | 2136.0 | 1069.2 | 1067.3 | A |
| I-565 | A | D | | | 2162.0 | 2163.1 | 2161.3 | A |
| I-566 | B | | | | 2193.9 | 1198.3 | 1196.3 | |
| I-567 | B | | | | 2219.9 | 1111.4 | 1109.3 | |
| I-568 | A | | | | 2092.9 | 2095.1 | 2093.1 | |
| I-569 | A | | | | 2092.9 | 2094.8 | 2092.6 | |
| I-570 | A | | | | 2108.9 | 2111.5 | 2110.0 | |
| I-571 | A | | | | 2108.9 | 2111.8 | 2109.9 | |
| I-572 | A | | | | 2152.8 | 2156.1 | 2153.8 | |
| I-573 | A | | | | 2152.8 | 2155.9 | 2153.7 | |
| I-574 | A | | | | 2092.9 | 2095.0 | 2092.8 | |
| I-575 | A | | | | 2092.9 | 2094.9 | 2092.6 | |
| I-576 | A | | | | 2108.9 | 2111.5 | 2110.0 | |
| I-577 | A | | | | 2108.9 | 1056.5 | 1054.8 | |
| I-578 | A | | | | 2152.8 | 2156.0 | 2153.8 | |
| I-579 | A | | | | 2152.8 | 2155.8 | 2154.4 | |
| I-580 | A | | | | 2092.9 | 2095.2 | 2093.3 | |
| I-581 | A | | | | 2092.9 | 2095.0 | 2093.2 | |
| I-582 | A | | | | 2104.9 | 1054.2 | 1052.4 | |
| I-583 | A | | | | 2104.9 | 2106.9 | 2104.8 | |
| I-584 | A | | | | 2099.9 | 2101.9 | 2100.1 | |
| I-585 | A | | | | 2099.9 | 2101.9 | 2099.8 | |
| I-586 | A | | | | 2099.9 | 2101.9 | 2099.8 | |
| I-587 | A | | | | 2099.9 | 2101.9 | 2100.0 | |
| I-588 | A | | | | 2088.9 | 2091.1 | 2088.8 | |
| I-589 | A | | | | 2088.9 | 2090.9 | 2089.0 | |
| I-590 | A | | | | 2088.9 | 2091.1 | 2089.6 | |
| I-591 | A | | | | 2088.9 | 2091.1 | 2088.8 | |
| I-592 | E | | | | 2086.9 | 2088.8 | 2087.1 | |
| I-593 | E | | | | 2086.9 | 2088.9 | 2087.0 | |
| I-594 | E | | | | 2117.0 | 2119.2 | 2117.2 | |
| I-595 | E | | | | 2117.0 | 2119.0 | 2117.1 | |
| I-596 | E | | | | 2117.0 | 2119.1 | 2116.8 | |
| I-597 | E | | | | 2117.0 | 2119.1 | 2117.0 | |
| I-598 | E | | | | 2117.0 | 2119.2 | 2117.3 | |
| I-599 | E | | | | 2117.0 | 2119.2 | 2116.9 | |
| I-600 | E | | | | 2117.0 | 2119.2 | 2117.1 | |
| I-601 | E | | | | 2117.0 | 2119.0 | 2117.0 | |
| I-602 | E | | | | 2117.0 | 2119.1 | 2116.9 | |
| I-603 | E | | | | 2117.0 | 2119.0 | 2117.0 | |
| I-604 | E | | | | 2117.0 | 2119.1 | 2116.9 | |
| I-605 | E | | | | 2117.0 | 2119.0 | 2117.2 | |
| I-606 | E | | | | 2186.0 | 2188.1 | 2186.2 | |
| I-607 | A | | | | 2158.0 | 2160.2 | 2158.1 | |

### Table E2. Certain Peptides and Compositions Thereof as Examples.

Peptides are stapled unless indicated otherwise (among other things, the present disclosure also provides unstapled versions of such peptides, optionally protected with one or more protection group (e.g., protection of N-terminus, C-terminus, side chains, etc.), and intermediates thereof). As appreciated by those skilled in the art, stapling may provide more than one stereoisomers (e.g., E/Z of double bonds and/or diastereomers). In some embodiments, a double bond in a staple is E. In some embodiments, a double bond in a staple is Z. In some embodiments, isomers (or combinations thereof) are listed separately (typically based on reverse phase HPLC peaks (e.g., detected by UV (e.g., at 220 nm) and/or MS) in the order of elution: each earlier eluted peak is assigned a smaller ID number than each later eluted peaks (if any); in some cases, a peak may contain two or more isomers; in some cases, isomers are not separated (or single isomer), e.g., when there is one peak on HPLC). Compositions utilized in various assays are typically of stapled peptides; the present disclosure also provides peptides prior to stapling and compositions thereof. A general HPLC method: Xselect CSH C18 column 1.7 um 2.1×50 mm 130 Å; Column temperature 40° C.; Flow 0.6 mL/min; 0.100 formic acid in both acetonitrile and water, 7.2 min gradient from 5 to 9500 acetonitrile. In some embodiments, a different gradient and/or a C8 column were used.

| 1 | Description |
|---|---|
| I-1 | Ac-PL3-Asp-Leu-B5-Asp-Asp-Ala-Ala-Phe-dLys*3-PyrS2-3Thi-BztA-GlnR*3-NH2 |
| I-2 | Ac-PL3-Asp-Leu-B5-Asp-Asp-Ala-Ala-Phe-dLys*3-PyrS2-3Thi-BztA-GlnR*3-NH2 |
| I-3 | Ac-PL3-Asp-Leu-B5-Asp-Asp-Ala-Ala-Phe-Lys*3-PyrS2-3Thi-BztA-GlnR*3-NH2 |
| I-4 | Ac-PL3-Asp-Leu-B5-Asp-Asp-Ala-Ala-Phe-Lys*3-PyrS2-3Thi-BztA-GlnR*3-NH2 |
| I-5 | Ac-PL3-Asp-Leu-B5-Asp-Asp-Ala-Ala-Phe-DGlnR*3-PyrS2-3Thi-BztA-Lys*3-NH2 |
| I-6 | Ac-PL3-Asp-Leu-B5-Asp-Asp-Ala-Ala-Phe-GlnR*3-PyrS2-3Thi-BztA-Lys*3-NH2 |
| I-7 | Ac-PL3-Asp-Leu-B5-Asp-Asp-Lys*3-Ala-Phe-GlnR*3-PyrS2-3Thi-BztA-Gln-NH2 |
| I-8 | Ac-PL3-Asp-Leu-B5-Asp-Asp-GlnR*3-Ala-Phe-Lys*3-PyrS2-3Thi-BztA-Gln-NH2 |
| I-9 | Ac-PL3-Asp-Leu-B5-Asp-3COOHF-Ala-Ala-Phe-dLys*3-PyrS2-3Thi-BztA-GlnR*3-NH2 |
| I-10 | Ac-PL3-Asp-Leu-B5-Asp-3COOHF-Ala-Ala-Phe-dLys*3-PyrS2-3Thi-BztA-GlnR*3-NH2 |
| I-11 | Ac-PL3-Asp-Leu-B5-Asp-3COOHF-Ala-Ala-Phe-Lys*3-PyrS2-3Thi-BztA-GlnR*3-NH2 |
| I-12 | Ac-PL3-Asp-Leu-B5-Asp-3COOHF-Ala-Ala-Phe-Lys*3-PyrS2-3Thi-BztA-GlnR*3-NH2 |
| I-13 | Ac-PL3-Asp-Leu-B5-Asp-3COOHF-Ala-Ala-Phe-DGlnR*3-PyrS2-3Thi-BztA-Lys*3-NH2 |
| I-14 | Ac-PL3-Asp-Leu-B5-Asp-3COOHF-Ala-Ala-Phe-DGlnR*3-PyrS2-3Thi-BztA-Lys*3-NH2 |
| I-15 | Ac-PL3-Asp-Leu-B5-Asp-3COOHF-Ala-Ala-Phe-GlnR*3-PyrS2-3Thi-BztA-Lys*3-NH2 |
| I-16 | Ac-PL3-Asp-Leu-B5-Asp-3COOHF-Ala-Ala-Phe-GlnR*3-PyrS2-3Thi-BztA-Lys*3-NH2 |
| I-17 | Ac-PL3-Asp-Npg-B5-Asp-Asp-Ala-Ala-Phe-TriAzLys*3-PyrS2-3Thi-BztA-sAla*3-NH2 |
| I-18 | Ac-PL3-Asp-Npg-B5-Asp-Asp-Ala-Ala-Phe-TriAzLys*3-PyrS2-3Thi-BztA-sAla*3-NH2 |
| I-19 | Ac-PL3-Asp-Npg-B5-Asp-3COOHF-Ala-Ala-Phe-TriAzLys*3-PyrS2-3Thi-BztA-sAla*3-NH2 |
| I-20 | Ac-PL3-Asp-Npg-B5-Asp-3COOHF-Ala-Ala-Phe-TriAzLys*3-PyrS2-3Thi-BztA-sAla*3-NH2 |
| I-21 | Ac-PL3-Asp-Npg-B5-Asp-3COOHF-Ala-Ala-Phe-TriAzLys*3-PyrS2-2F3MeF-BztA-sAla*3- |
| | NH2 |
| I-22 | Ac-PL3-Asp-Npg-B5-Asp-3COOHF-Ala-Ala-Phe-TriAzLys*3-PyrS2-2F3MeF-BztA-sAla*3- |
| | NH2 |
| I-23 | Ac-PL3-Asp-Npg-B5-Asp-Asp-Ala-Ala-Phe-sAla*3-PyrS2-3Thi-BztA-TriAzLys*3-NH2 |
| I-24 | Ac-PL3-Asp-Npg-B5-Asp-3COOHF-Ala-Ala-Phe-sAla*3-PyrS2-3Thi-BztA-TriAzLys*3-NH2 |
| I-25 | Ac-PL3-Asp-Npg-B5-Asp-3COOHF-Ala-Ala-Phe-sAla*3-PyrS2-2F3MeF-BztA-TriAzLys*3- |
| | NH2 |
| I-26 | Ac-PL3-Asp-Npg-B5-Asp-Asp-sAla*3-Ala-Phe-TriAzLys*3-PyrS2-3Thi-BztA-Gln-NH2 |
| I-27 | Ac-PL3-Asp-Npg-B5-Asp-3COOHF-sAla*3-Ala-Phe-TriAzLys*3-PyrS2-3Thi-BztA-Gln-NH2 |
| I-28 | Ac-PL3-Asp-Npg-B5-Asp-3COOHF-sAla*3-Ala-Phe-TriAzLys*3-PyrS2-2F3MeF-BztA-Gln- |
| | NH2 |
| I-29 | Ac-PL3-Asp-Npg-B5-Asp-Asp-TriAzLys*3-Ala-Phe-sAla*3-PyrS2-3Thi-BztA-Gln-NH2 |
| I-30 | Ac-PL3-Asp-Npg-B5-Asp-3COOHF-TriAzLys*3-Ala-Phe-sAla*3-PyrS2-3Thi-BztA-Gln-NH2 |
| I-31 | Ac-PL3-Asp-Npg-B5-Asp-3COOHF-TriAzLys*3-Ala-Phe-sAla*3-PyrS2-2F3MeF-BztA-Gln- |
| | NH2 |
| I-32 | Ac-PL3-Asp-Npg-B5-Asp-3COOHF-Ala-Ala-Phe-Lys*3-PyrS2-3Thi-BztA-GlnR*3-NH2 |
| I-33 | Ac-PL3-Asp-Npg-B5-Asp-3COOHF-Ala-Ala-Phe-GlnR*3-PyrS2-3Thi-BztA-Lys*3-NH2 |
| I-34 | Ac-PL3-Asp-Npg-B5-Asp-3COOHF-Ala-Ala-Phe-Lys*3-PyrS2-3Thi-BztA-AsnR*3-NH2 |
| I-35 | Ac-PL3-Asp-Npg-B5-Asp-3COOHF-Ala-Ala-Phe-AsnR*3-PyrS2-3Thi-BztA-Lys*3-NH2 |
| I-36 | Ac-PL3-Asp-Npg-B5-Asp-3COOHF-Ala-Ala-Phe-AsnR*3-PyrS2-3Thi-BztA-Lys*3-NH2 |
| I-37 | Ac-PL3-Asp-Npg-B5-Asp-3COOHF-Ala-Ala-Phe-Orn*3-PyrS2-3Thi-BztA-GlnR*3-NH2 |
| I-38 | Ac-PL3-Asp-Npg-B5-Asp-3COOHF-Ala-Ala-Phe-GlnR*3-PyrS2-3Thi-BztA-Orn*3-NH2 |
| I-39 | Ac-PL3-Asp-Npg-B5-Asp-3COOHF-Ala-Ala-Phe-Orn*3-PyrS2-3Thi-BztA-AsnR*3-NH2 |
| I-40 | Ac-PL3-Asp-Npg-B5-Asp-3COOHF-Ala-Ala-Phe-AsnR*3-PyrS2-3Thi-BztA-Orn*3-NH2 |
| I-41 | Ac-PL3-Asp-Npg-B5-Asp-3COOHF-Ala-Ala-Phe-AsnR*3-PyrS2-3Thi-BztA-Orn*3-NH2 |
| I-42 | Ac-PL3-Asp-Npg-B5-Asp-3COOHF-Ala-Ala-Phe-Lys*3-PyrS2-3Thi-BztA-GlnR*3-Ala-NH2 |
| I-43 | Ac-PL3-Asp-Npg-B5-Asp-3COOHF-Ala-Ala-Phe-GlnR*3-PyrS2-3Thi-BztA-Lys*3-Ala-NH2 |
| I-44 | Ac-PL3-Asp-Npg-B5-Asp-3COOHF-Ala-Ala-Phe-AsnR*3-PyrS2-3Thi-BztA-Lys*3-Ala-NH2 |
| I-45 | Ac-PL3-Asp-Npg-B5-Asp-3COOHF-[isophthalate]-Lys-Ala-Phe-Leu-PyrS2-3Thi-BztA-Lys-NH2 |
| I-46 | Ac-PL3-Asp-Npg-B5-Asp-3COOHF-[succinate]-Lys-Ala-Phe-Leu-PyrS2-3Thi-BztA-Lys-NH2 |
| I-47 | Ac-PL3-Asp-Npg-B5-Asp-3COOHF-[Me2Mal]-Lys-Ala-Phe-Leu-PyrS2-3Thi-BztA-Lys-NH2 |
| I-48 | Ac-PL3-Asp-Npg-B5-Asp-3COOHF-[diphenate]-Lys-Ala-Phe-Leu-PyrS2-3Thi-BztA-Lys-NH2 |
| I-49 | Ac-PL3-Asp-Npg-B5-Asp-3COOHF-[Biphen33COOH]-Lys-Ala-Phe-Leu-PyrS2-3Thi-BztA-Lys- |
| | NH2 |
| I-50 | Ac-PL3-Asp-Npg-B5-Asp-3COOHF-[isophthalate]-Lys-Ala-Phe-Leu-PyrS2-2ClF-BztA-Lys-NH2 |
| I-51 | Ac-PL3-Asp-Npg-B5-Asp-3COOHF-[succinate]-Lys-Ala-Phe-Leu-PyrS2-2ClF-BztA-Lys-NH2 |
| I-52 | Ac-PL3-Asp-Npg-B5-Asp-3COOHF-[Me2Mal]Lys-Ala-Phe-Leu-PyrS2-2ClF-BztA-Lys-NH2 |
| I-53 | Ac-PL3-Asp-Npg-B5-Asp-3COOHF-[diphenate]Lys-Ala-Phe-Leu-PyrS2-2ClF-BztA-Lys-NH2 |
| I-54 | Ac-PL3-Asp-Npg-B5-Asp-3COOHF-[Biphen33COOH]Lys-Ala-Phe-Leu-PyrS2-2ClF-BztA-Lys- |
| | NH2 |
| I-55 | Ac-PL3-Asp-Npg-B5-Asp-3COOHF-Ala-Ala-Phe-Lys*3-PyrS2-2F3MeF-BztA-GlnR*3-NH2 |
| I-56 | Ac-PL3-Asp-Npg-B5-Asp-3COOHF-Ala-Ala-Phe-Lys*3-PyrS2-2F3MeF-BztA-AsnR*3-NH2 |
| I-57 | Ac-PL3-Asp-Npg-B5-Asp-3COOHF-Ala-Ala-Phe-Lys*3-PyrS2-2F3MeF-BztA-GlnR*3-Ala-NH2 |
| I-58 | Ac-PL3-Asp-Npg-B5-Asp-3COOHF-Ala-Ala-Phe-Lys*3-PyrS2-2F3MeF-BztA-AsnR*3-Ala- |
| | NH2 |
| I-59 | Ac-PL3-Asp-Npg-B5-Asp-3COOHF-Ala-Ala-Phe-Lys*3-PyrS2-2F3MeF-BztA-AsnR*3-Ala- |
| | NH2 |
| I-60 | Ac-PL3-Asp-Npg-B5-Asp-3COOHF-Gln-Ala-Phe-Lys*3-PyrS2-2F3MeF-BztA-GlnR*3-Ala-NH2 |
| I-61 | Ac-PL3-Asp-Npg-B5-Asp-3COOHF-Gln-Ala-Phe-Lys*3-PyrS2-2F3MeF-BztA-AsnR*3-Ala- |
| | NH2 |
| I-62 | Ac-PL3-Asp-Npg-B5-Asp-3COOHF-Ala-Ala-Phe-Lys*3-PyrS2-3Thi-BztA-GlnR*3-Throl |
| I-63 | Ac-PL3-Asp-Npg-B5-Asp-3COOHF-Ala-Ala-Phe-Lys*3-PyrS2-3Thi-BztA-GlnR*3-Throl |
| I-64 | Ac-PL3-Asp-Npg-B5-Asp-3COOHF-Ala-Ala-Phe-Lys*3-PyrS2-3Thi-BztA-GlnR*3-Prool |
| I-65 | Ac-PL3-Asp-Npg-B5-Asp-3COOHF-Ala-Ala-Phe-Lys*3-PyrS2-3Thi-BztA-GlnR*3-Alaol |
| I-66 | Ac-PL3-Asp-Npg-B5-Asp-3COOHF-Aib-Ala-Phe-Lys*3-PyrS2-3Thi-BztA-GlnR*3-Ala-NH2 |
| I-67 | Ac-PL3-Asp-Npg-B5-Asp-3COOHF-Aib-Ala-Phe-Lys*3-PyrS2-3Thi-BztA-GlnR*3-Ala-NH2 |
| I-68 | Ac-PL3-Asp-Npg-B5-Asp-3COOHF-Aib-Ala-Phe-Lys*3-PyrS2-2F3MeF-BztA-GlnR*3-Ala-NH2 |
| I-69 | Ac-PL3-Asp-Npg-B5-Asp-3COOHF-Aib-Ala-Phe-Lys*3-PyrS2-2F3MeF-BztA-GlnR*3-Ala-NH2 |
| I-70 | Ac-PL3-Asp-Npg-B5-Asp-3COOHF-nLeu-Ala-Phe-Lys*3-PyrS2-3Thi-BztA-GlnR*3-Ala-NH2 |
| I-71 | Ac-PL3-Asp-Npg-B5-Asp-3COOHF-nLeu-Ala-Phe-Lys*3-PyrS2-2F3MeF-BztA-GlnR*3-Ala- |
| | NH2 |
| I-72 | Ac-PL3-Asp-Npg-B5-Asp-3COOHF-Thr-Ala-Phe-Lys*3-PyrS2-3Thi-BztA-GlnR*3-Ala-NH2 |
| I-73 | Ac-PL3-Asp-Npg-B5-Asp-3COOHF-Thr-Ala-Phe-Lys*3-PyrS2-2F3MeF-BztA-GlnR*3-Ala-NH2 |
| I-74 | Ac-PL3-Asp-GlnR**3-B5-Asp-3COOHF-Lys**3-Ala-Phe-Lys*3-PyrS2-3Thi-BztA-GlnR*3-Ala- |
| | NH2 |
| I-75 | Ac-PL3-Asp-GlnR**3-B5-Asp-3COOHF-Lys**3-Ala-Phe-Lys*3-PyrS2-2F3MeF-BztA-GlnR*3- |
| | Ala-NH2 |
| I-76 | Ac-PL3-Asp-Npg-B5-Asp-3COOHF-Ala-Ala-Phe-Lys*3-PyrS2-3Thi-BztA-GlnR*3-Ala-NH2 |
| I-77 | Ac-PL3-Asp-Npg-B5-Asp-3COOHF-Ala-Ala-Phe-Lys*3-PyrS2-2F3MeF-BztA-GlnR*3-Ala-NH2 |
| I-78 | Ac-PL3-Asp-GlnR**3-B5-Asp-3COOHF-Lys**3-Ala-Phe-Lys*3-PyrS2-3Thi-BztA-GlnR*3-Ala- |
| | NH2 |
| I-79 | Ac-PL3-Asp-GlnR**3-B5-Asp-3COOHF-Lys**3-Ala-Phe-Lys*3-PyrS2-2F3MeF-BztA-GlnR*3- |
| | Ala-NH2 |
| I-80 | Ac-PL3-Asn-Npg-B5-Asp-3COOHF-Ala-Ala-Phe-Lys*3-PyrS2-3Thi-BztA-GlnR*3-Ala-NH2 |
| I-81 | Ac-PL3-Hse-Npg-B5-Asp-3COOHF-Ala-Ala-Phe-Lys*3-PyrS2-3Thi-BztA-GlnR*3-Ala-NH2 |
| I-82 | Ac-PL3-Asp-Npg-B5-Asn-3COOHF-Ala-Ala-Phe-Lys*3-PyrS2-3Thi-BztA-GlnR*3-Ala-NH2 |
| I-83 | Ac-PL3-Asp-Npg-B5-Hse-3COOHF-Ala-Ala-Phe-Lys*3-PyrS2-3Thi-BztA-GlnR*3-Ala-NH2 |
| I-84 | Ac-PL3-Asn-Npg-B5-Asp-3COOHF-Ser-Ala-Phe-Lys*3-PyrS2-3Thi-BztA-GlnR*3-Ala-NH2 |
| I-85 | Ac-PL3-Hse-Npg-B5-Asp-3COOHF-Ser-Ala-Phe-Lys*3-PyrS2-3Thi-BztA-GlnR*3-Ala-NH2 |
| I-86 | Ac-PL3-Asp-Npg-B5-Asn-3COOHF-Ser-Ala-Phe-Lys*3-PyrS2-3Thi-BztA-GlnR*3-Ala-NH2 |
| I-87 | Ac-PL3-Asp-Npg-B5-Hse-3COOHF-Ser-Ala-Phe-Lys*3-PyrS2-3Thi-BztA-GlnR*3-Ala-NH2 |
| I-88 | Ac-PL3-Asn-Npg-B5-Asp-3COOHF-Gln-Ala-Phe-Lys*3-PyrS2-3Thi-BztA-GlnR*3-Ala-NH2 |
| I-89 | Ac-PL3-Hse-Npg-B5-Asp-3COOHF-Gln-Ala-Phe-Lys*3-PyrS2-3Thi-BztA-GlnR*3-Ala-NH2 |
| I-90 | Ac-PL3-Asp-Npg-B5-Asn-3COOHF-Gln-Ala-Phe-Lys*3-PyrS2-3Thi-BztA-GlnR*3-Ala-NH2 |
| I-91 | Ac-PL3-Asp-Npg-B5-Hse-3COOHF-Gln-Ala-Phe-Lys*3-PyrS2-3Thi-BztA-GlnR*3-Ala-NH2 |
| I-92 | Ac-PL3-Asp-Npg-B5-Asp-3COOHF-GlnR-Ala-Phe-Leu-PyrS2-2F3MeF-BztA- |
| | [diaminobutane]GlnR-Ala-NH2 |
| I-93 | Ac-PL3-Asp-Npg-B5-Asp-3COOHF-GlnR-Ala-Phe-Leu-PyrS2-2F3MeF-BztA- |
| | [4aminopiperidine]GlnR-Ala-NH2 |
| I-94 | Ac-PL3-Asp-Npg-B5-Asp-3COOHF-[4aminopiperidine]GlnR-Ala-Phe-Leu-PyrS2-2F3MeF- |
| | BztA-GlnR-Ala-NH2 |
| I-95 | Ac-PL3-Asp-Npg-B5-Asp-3COOHF-GlnR-Ala-Phe-Leu-PyrS2-2F3MeF-BztA- |
| | [4mampiperidine]GlnR-Ala-NH2 |
| I-96 | Ac-PL3-Asp-Npg-B5-Asp-3COOHF-GlnR-Ala-Phe-Leu-PyrS2-2F3MeF-BztA- |
| | [4mampiperidine]GlnR-Ala-NH2 |
| I-97 | Ac-PL3-Asp-Cha-B5-Asp-3COOHF-Ala-Ala-Phe-Lys*3-PyrS2-2F3MeF-BztA-GlnR*3-Ala-NH2 |
| I-98 | Ac-PL3-Asp-Cha-B5-Asp-3COOHF-Ala-Ala-Phe-Lys*3-PyrS2-2F3MeF-BztA-GlnR*3-Ala-NH2 |
| I-99 | Ac-PL3-Asp-Npg-B5-Asp-3COOHF-Npg-Ala-Phe-Lys*3-PyrS2-2F3MeF-BztA-GlnR*3-Ala- |
| | NH2 |
| I-100 | Ac-PL3-Asp-Npg-B5-Asp-3COOHF-Npg-Ala-Phe-Lys*3-PyrS2-2F3MeF-BztA-GlnR*3-Ala- |
| | NH2 |
| I-101 | Ac-PL3-Asp-Npg-B5-Asp-3COOHF-Cha-Ala-Phe-Lys*3-PyrS2-2F3MeF-BztA-GlnR*3-Ala- |
| | NH2 |
| I-102 | Ac-PL3-Asp-Npg-B5-Asp-3COOHF-Cha-Ala-Phe-Lys*3-PyrS2-2F3MeF-BztA-GlnR*3-Ala- |
| | NH2 |
| I-103 | Ac-PL3-Asp-Cha-B5-Asp-3COOHF-Cha-Ala-Phe-Lys*3-PyrS2-2F3MeF-BztA-GlnR*3-Ala-NH2 |
| I-104 | Ac-PL3-Asp-Npg-B5-Asp-3COOHF-Ala-Ala-Phe-Lys*3-PyrS2-2F3MeF-BztA-GlnR*3-Leu- |
| | NH2 |
| I-105 | Ac-PL3-Asp-Npg-B5-Asp-3COOHF-Ala-Ala-Phe-Lys*3-PyrS2-2F3MeF-BztA-GlnR*3-Leu- |
| | NH2 |
| I-106 | Ac-PL3-Asp-Cha-B5-Asp-3COOHF-Ala-Ala-Phe-Lys*3-PyrS2-2F3MeF-BztA-GlnR*3-Leu-NH2 |
| I-107 | Ac-PL3-Asp-Cha-B5-Asp-3COOHF-Ala-Ala-Phe-Lys*3-PyrS2-2F3MeF-BztA-GlnR*3-Leu-NH2 |
| I-108 | Ac-PL3-Asp-Npg-B5-Asp-3COOHF-Npg-Ala-Phe-Lys*3-PyrS2-2F3MeF-BztA-GlnR*3-Leu- |
| | NH2 |
| I-109 | Ac-PL3-Asp-Npg-B5-Asp-3COOHF-Npg-Ala-Phe-Lys*3-PyrS2-2F3MeF-BztA-GlnR*3-Leu- |
| | NH2 |
| I-110 | Ac-PL3-Asp-Npg-B5-Asp-3COOHF-Cha-Ala-Phe-Lys*3-PyrS2-2F3MeF-BztA-GlnR*3-Leu- |
| | NH2 |
| I-111 | Ac-PL3-Asp-Npg-B5-Asp-3COOHF-Cha-Ala-Phe-Lys*3-PyrS2-2F3MeF-BztA-GlnR*3-Leu- |
| | NH2 |
| I-112 | Ac-PL3-Asp-Npg-B5-Asp-TfeGA-Ala-Ala-Phe-Lys*3-PyrS2-2F3MeF-BztA-GlnR*3-Ala-NH2 |
| I-113 | Ac-PL3-Asn-Npg-B5-Asp-TfeGA-Ala-Ala-Phe-Lys*3-PyrS2-2F3MeF-BztA-GlnR*3-Ala-NH2 |
| I-114 | Ac-PL3-Asn-Npg-B5-Asp-TfeGA-Ala-Ala-Phe-Lys*3-PyrS2-2F3MeF-BztA-GlnR*3-Ala-NH2 |
| I-115 | Ac-PL3-Hse-Npg-B5-Asp-TfeGA-Ala-Ala-Phe-Lys*3-PyrS2-2F3MeF-BztA-GlnR*3-Ala-NH2 |
| I-116 | Ac-PL3-Hse-Npg-B5-Asp-TfeGA-Ala-Ala-Phe-Lys*3-PyrS2-2F3MeF-BztA-GlnR*3-Ala-NH2 |
| I-117 | Ac-PL3-Asp-Npg-B5-Asn-TfeGA-Ala-Ala-Phe-Lys*3-PyrS2-2F3MeF-BztA-GlnR*3-Ala-NH2 |
| I-118 | Ac-PL3-Asp-Npg-B5-Hse-TfeGA-Ala-Ala-Phe-Lys*3-PyrS2-2F3MeF-BztA-GlnR*3-Ala-NH2 |
| I-119 | Ac-PL3-Asp-Npg-B5-Hse-TfeGA-Ala-Ala-Phe-Lys*3-PyrS2-2F3MeF-BztA-GlnR*3-Ala-NH2 |
| I-120 | Ac-PL3-Asp-Npg-B5-Asp-TfeGA-Aib-Ala-Phe-Lys*3-PyrS2-2F3MeF-BztA-GlnR*3-Ala-NH2 |
| I-121 | Ac-PL3-Asp-Npg-B5-Asp-TfeGA-Aib-Ala-Phe-Lys*3-PyrS2-2F3MeF-BztA-GlnR*3-Ala-NH2 |
| I-122 | Ac-PL3-Asp-Npg-B5-Asp-TfeGA-Leu-Ala-Phe-Lys*3-PyrS2-2F3MeF-BztA-GlnR*3-Ala-NH2 |
| I-123 | Ac-PL3-Asp-Npg-B5-Asp-TfeGA-Phe-Ala-Phe-Lys*3-PyrS2-2F3MeF-BztA-GlnR*3-Ala-NH2 |
| I-124 | Ac-PL3-Asp-Npg-B5-Asp-TfeGA-Val-Ala-Phe-Lys*3-PyrS2-2F3MeF-BztA-GlnR*3-Ala-NH2 |
| I-125 | Ac-PL3-Asp-Npg-B5-Asp-TfeGA-Val-Ala-Phe-Lys*3-PyrS2-2F3MeF-BztA-GlnR*3-Ala-NH2 |
| I-126 | Ac-PL3-Asp-Npg-B5-Asp-TfeGA-Ala-Ala-Phe-Lys*3-PyrS2-2F3MeF-BztA-GlnR*3-Aib-NH2 |
| I-127 | Ac-PL3-Asp-Npg-B5-Asp-TfeGA-Ala-Ala-Phe-Lys*3-PyrS2-2F3MeF-BztA-GlnR*3-Aib-NH2 |
| I-128 | Ac-PL3-Asp-Npg-B5-Asp-TfeGA-Ala-Ala-Phe-Lys*3-PyrS2-2F3MeF-BztA-GlnR*3-Leu-NH2 |
| I-129 | Ac-PL3-Asp-Npg-B5-Asp-TfeGA-Ala-Ala-Phe-Lys*3-PyrS2-2F3MeF-BztA-GlnR*3-Val-NH2 |
| I-130 | Ac-PL3-Asp-Npg-B5-Asp-3COOHF-GlnR-Ala-Phe-Leu-PyrS2-2F3MeF-BztA- |
| | [39N2spiroundecane]GlnR-Ala-NH2 |
| I-131 | Ac-PL3-Asp-Npg-B5-Asp-3COOHF-GlnR-Ala-Phe-Leu-PyrS2-2F3MeF-BztA- |
| | [29N2spiroundecane]GlnR-Ala-NH2 |
| I-132 | Ac-PL3-Asp-Npg-B5-Asp-3COOHF-[29N2spiroundecane]GlnR-Ala-Phe-Leu-PyrS2-2F3MeF- |
| | BztA-GlnR-Ala-NH2 |
| I-133 | Ac-PL3-Asp-Npg-B5-Asp-3COOHF-GlnR-Ala-Phe-Leu-PyrS2-2F3MeF-BztA- |
| | [39N2spiroundecane]GlnR-Ala-NH2 |
| I-134 | Ac-PL3-Asp-Npg-B5-Asp-3COOHF-GlnR-Ala-Phe-Leu-PyrS2-2F3MeF-BztA- |
| | [29N2spiroundecane]GlnR-Ala-NH2 |
| I-135 | Ac-PL3-Asp-Npg-B5-Asp-3COOHF-[29N2spiroundecane]GlnR-Ala-Phe-Leu-PyrS2-2F3MeF- |
| | BztA-GlnR-Ala-NH2 |
| I-136 | Ac-PL3-Asp-Npg-B5-Asp-3COOHF-Ala-Ala-Phe-Lys*3-PyrS2-2F3MeF-BztA-GlnR*3-Leu-Ser- |
| | NH2 |
| I-137 | Ac-PL3-Asp-Npg-B5-Asp-3COOHF-nLeu-Ala-Phe-Lys*3-PyrS2-2F3MeF-BztA-GlnR*3-Leu- |
| | Ser-NH2 |
| I-138 | Ac-PL3-Asp-Npg-B5-Asp-3COOHF-Aib-Ala-Phe-Lys*3-PyrS2-2F3MeF-BztA-GlnR*3-Leu-Ser- |
| | NH2 |
| I-139 | Ac-PL3-Asp-Npg-B5-Asp-3COOHF-Cpg-Ala-Phe-Lys*3-PyrS2-2F3MeF-BztA-GlnR*3-Leu-Ser- |
| | NH2 |
| I-140 | Ac-PL3-Asp-Npg-B5-Asp-3COOHF-Cbg-Ala-Phe-Lys*3-PyrS2-2F3MeF-BztA-GlnR*3-Leu-Ser- |
| | NH2 |
| I-141 | Ac-PL3-Asp-Npg-B5-Asp-3COOHF-CyLeu-Ala-Phe-Lys*3-PyrS2-2F3MeF-BztA-GlnR*3-Leu- |
| | Ser-NH2 |
| I-142 | Ac-PL3-Asn-Npg-B5-Asp-3COOHF-Ala-Ala-Phe-Lys*3-PyrS2-2F3MeF-BztA-GlnR*3-Leu-Ser- |
| | NH2 |
| I-143 | Ac-PL3-Asn-Npg-B5-Asp-3COOHF-nLeu-Ala-Phe-Lys*3-PyrS2-2F3MeF-BztA-GlnR*3-Leu- |
| | Ser-NH2 |
| I-144 | Ac-PL3-Asn-Npg-B5-Asp-3COOHF-Aib-Ala-Phe-Lys*3-PyrS2-2F3MeF-BztA-GlnR*3-Leu-Ser- |
| | NH2 |
| I-145 | Ac-PL3-Hse-Npg-B5-Asp-3COOHF-Ala-Ala-Phe-Lys*3-PyrS2-2F3MeF-BztA-GlnR*3-Leu-Ser- |
| | NH2 |
| I-146 | Ac-PL3-Hse-Npg-B5-Asp-3COOHF-nLeu-Ala-Phe-Lys*3-PyrS2-2F3MeF-BztA-GlnR*3-Leu- |
| | Ser-NH2 |
| I-147 | Ac-PL3-Hse-Npg-B5-Asp-3COOHF-Aib-Ala-Phe-Lys*3-PyrS2-2F3MeF-BztA-GlnR*3-Leu-Ser- |
| | NH2 |
| I-148 | Ac-PL3-Asn-Cha-B5-Asp-Gln-Hse-Ala-Phe-GlnR*3-PyrS2-Phe-BztA-Lys*3-NH2 |
| I-149 | Ac-PL3-Asn-Cha-B5-Asp-Gln-iPrLys-Ala-Phe-GlnR*3-PyrS2-Phe-BztA-Lys*3-NH2 |
| I-150 | Ac-PL3-Asn-Cha-B5-Asp-Thr-iPrLys-Ala-Phe-GlnR*3-PyrS2-Phe-BztA-Lys*3-NH2 |
| I-151 | Ac-PL3-Asn-Cha-B5-Asp-Gln-Hse-Ala-Phe-GlnR*3-PyrS2-3Thi-BztA-Lys*3-NH2 |
| I-152 | Ac-PL3-Asn-Cha-B5-Asp-Gln-iPrLys-Ala-Phe-GlnR*3-PyrS2-3Thi-BztA-Lys*3-NH2 |
| I-153 | Ac-PL3-Asn-Cha-B5-Asp-Gln-Hse-Ala-Phe-GlnR*3-PyrS2-Phe-BztA-Lys*3-NH2 |
| I-154 | Ac-PL3-Asn-Cha-B5-Asp-Gln-iPrLys-Ala-Phe-GlnR*3-PyrS2-Phe-BztA-Lys*3-NH2 |
| I-155 | Ac-PL3-Asn-Cha-B5-Asp-Thr-Hse-Ala-Phe-GlnR*3-PyrS2-Phe-BztA-Lys*3-NH2 |
| I-156 | Ac-PL3-Asn-Cha-B5-Asp-Thr-iPrLys-Ala-Phe-GlnR*3-PyrS2-Phe-BztA-Lys*3-NH2 |
| I-157 | Ac-PL3-Asn-Cha-B5-Asp-Gln-Hse-Ala-Phe-GlnR*3-PyrS2-3Thi-BztA-Lys*3-NH2 |
| I-158 | Ac-PL3-Asn-Cha-B5-Asp-Gln-iPrLys-Ala-Phe-GlnR*3-PyrS2-3Thi-BztA-Lys*3-NH2 |
| I-159 | Ac-PL3-Asn-Npg-B5-Asp-3COOHF-Aib-Ala-Phe-Lys*3-PyrS2-3Thi-BztA-GlnR*3-Ala-NH2 |
| I-160 | Ac-PL3-Ser-Npg-B5-Asp-3COOHF-Aib-Ala-Phe-Lys*3-PyrS2-3Thi-BztA-GlnR*3-Ala-NH2 |
| I-161 | Ac-PL3-Thr-Npg-B5-Asp-3COOHF-Aib-Ala-Phe-Lys*3-PyrS2-3Thi-BztA-GlnR*3-Ala-NH2 |
| I-162 | Ac-PL3-Hse-Npg-B5-Asp-3COOHF-Aib-Ala-Phe-Lys*3-PyrS2-3Thi-BztA-GlnR*3-Ala-NH2 |
| I-163 | Ac-PL3-aThr-Npg-B5-Asp-3COOHF-Aib-Ala-Phe-Lys*3-PyrS2-3Thi-BztA-GlnR*3-Ala-NH2 |
| I-164 | Ac-PL3-MeAsn-Npg-B5-Asp-3COOHF-Aib-Ala-Phe-Lys*3-PyrS2-3Thi-BztA-GlnR*3-Ala-NH2 |
| I-165 | Ac-PL3-Asp-Npg-B5-Asn-3COOHF-Aib-Ala-Phe-Lys*3-PyrS2-3Thi-BztA-GlnR*3-Ala-NH2 |
| I-166 | Ac-PL3-Asp-Npg-B5-Ser-3COOHF-Aib-Ala-Phe-Lys*3-PyrS2-3Thi-BztA-GlnR*3-Ala-NH2 |
| I-167 | Ac-PL3-Asp-Npg-B5-Thr-3COOHF-Aib-Ala-Phe-Lys*3-PyrS2-3Thi-BztA-GlnR*3-Ala-NH2 |
| I-168 | Ac-PL3-Asp-Npg-B5-Hse-3COOHF-Aib-Ala-Phe-Lys*3-PyrS2-3Thi-BztA-GlnR*3-Ala-NH2 |
| I-169 | Ac-PL3-Asp-Npg-B5-aThr-3COOHF-Aib-Ala-Phe-Lys*3-PyrS2-3Thi-BztA-GlnR*3-Ala-NH2 |
| I-170 | Ac-PL3-Asp-Npg-B5-MeAsn-3COOHF-Aib-Ala-Phe-Lys*3-PyrS2-3Thi-BztA-GlnR*3-Ala-NH2 |
| I-171 | Ac-PL3-Asn-Npg-B5-Asp-3COOHF-Aib-Ala-Phe-Lys*3-PyrS2-3Thi-BztA-GlnR*3-Ala-NH2 |
| I-172 | Ac-PL3-Ser-Npg-B5-Asp-3COOHF-Aib-Ala-Phe-Lys*3-PyrS2-3Thi-BztA-GlnR*3-Ala-NH2 |
| I-173 | Ac-PL3-Thr-Npg-B5-Asp-3COOHF-Aib-Ala-Phe-Lys*3-PyrS2-3Thi-BztA-GlnR*3-Ala-NH2 |
| I-174 | Ac-PL3-Hse-Npg-B5-Asp-3COOHF-Aib-Ala-Phe-Lys*3-PyrS2-3Thi-BztA-GlnR*3-Ala-NH2 |
| I-175 | Ac-PL3-aThr-Npg-B5-Asp-3COOHF-Aib-Ala-Phe-Lys*3-PyrS2-3Thi-BztA-GlnR*3-Ala-NH2 |
| I-176 | Ac-PL3-MeAsn-Npg-B5-Asp-3COOHF-Aib-Ala-Phe-Lys*3-PyrS2-3Thi-BztA-GlnR*3-Ala-NH2 |
| I-177 | Ac-PL3-Asp-Npg-B5-Asn-3COOHF-Aib-Ala-Phe-Lys*3-PyrS2-3Thi-BztA-GlnR*3-Ala-NH2 |
| I-178 | Ac-PL3-Asp-Npg-B5-Ser-3COOHF-Aib-Ala-Phe-Lys*3-PyrS2-3Thi-BztA-GlnR*3-Ala-NH2 |
| I-179 | Ac-PL3-Asp-Npg-B5-Thr-3COOHF-Aib-Ala-Phe-Lys*3-PyrS2-3Thi-BztA-GlnR*3-Ala-NH2 |
| I-180 | Ac-PL3-Asp-Npg-B5-Hse-3COOHF-Aib-Ala-Phe-Lys*3-PyrS2-3Thi-BztA-GlnR*3-Ala-NH2 |
| I-181 | Ac-PL3-Asp-Npg-B5-aThr-3COOHF-Aib-Ala-Phe-Lys*3-PyrS2-3Thi-BztA-GlnR*3-Ala-NH2 |
| I-182 | Ac-PL3-Asp-Npg-B5-MeAsn-3COOHF-Aib-Ala-Phe-Lys*3-PyrS2-3Thi-BztA-GlnR*3-Ala-NH2 |
| I-183 | Ac-PL3-Asp-Npg-B5-Asp-3COOHF-Ala-Ala-Phe-Lys*3-PyrS2-3Thi-BztA-hGlnR*3-Ala-NH2 |
| I-184 | Ac-PL3-Asp-Npg-B5-Asp-3COOHF-Ala-Ala-Phe-Lys*3-PyrS2-2F3MeF-BztA-hGlnR*3-Ala- |
| | NH2 |
| I-185 | Ac-PL3-Asp-Npg-B5-Asp-3COOHF-Ala-Ala-Phe-hGlnR*3-PyrS2-3Thi-BztA-Lys*3-Ala-NH2 |
| I-186 | Ac-PL3-Asp-Npg-B5-Asp-3COOHF-Ala-Ala-Phe-hGlnR*3-PyrS2-2F3MeF-BztA-Lys*3-Ala- |
| | NH2 |
| I-187 | Ac-PL3-Asp-Npg-B5-Asp-3COOHF-Ala-Ala-Phe-iPrLys*3-PyrS2-3Thi-BztA-hGlnR*3-Ala- |
| | NH2 |
| I-188 | Ac-PL3-Asp-Npg-B5-Asp-3COOHF-Ala-Ala-Phe-iPrLys*3-PyrS2-2F3MeF-BztA-hGlnR*3-Ala- |
| | NH2 |
| I-189 | Ac-PL3-Asp-Npg-B5-Asp-3COOHF-Ala-Ala-Phe-hGlnR*3-PyrS2-2F3MeF-BztA-iPrLys*3-Ala- |
| | NH2 |
| I-190 | Ac-PL3-Asp-Npg-B5-Asp-3COOHF-Ala-Ala-Phe-iPrLys*3-PyrS2-3Thi-BztA-GlnR*3-Ala-NH2 |
| I-191 | Ac-PL3-Asp-Npg-B5-Asp-3COOHF-Ala-Ala-Phe-iPrLys*3-PyrS2-2F3MeF-BztA-GlnR*3-Ala- |
| | NH2 |
| I-192 | Ac-PL3-Asp-Npg-B5-Asp-3COOHF-Ala-Ala-Phe-GlnR*3-PyrS2-2F3MeF-BztA-iPrLys*3-Ala- |
| | NH2 |
| I-193 | Ac-PL3-Asp-Npg-B5-Asp-20H3COOHF-Aib-Ala-Phe-Lys*3-PyrS2-2F3MeF-BztA-GlnR*3-Ala- |
| | NH2 |
| I-194 | Ac-PL3-Asp-Npg-B5-Asp-40H3COOHF-Aib-Ala-Phe-Lys*3-PyrS2-2F3MeF-BztA-GlnR*3-Ala- |
| | NH2 |
| I-195 | Ac-PL3-Asp-Npg-B5-Asp-[CH2CMe2CO2H]TriAzDap-Aib-Ala-Phe-Lys*3-PyrS2-2F3MeF- |
| | BztA-GlnR*3-Ala-NH2 |
| I-196 | Ac-PL3-Asp-Npg-B5-Asp-4COOHF-Aib-Ala-Phe-Lys*3-PyrS2-2F3MeF-BztA-GlnR*3-Ala-NH2 |
| I-197 | Ac-PL3-Asp-Npg-B5-Asp-2COOHF-Aib-Ala-Phe-Lys*3-PyrS2-2F3MeF-BztA-GlnR*3-Ala-NH2 |
| I-198 | Ac-PL3-Asp-Npg-B5-Asp-Glu-Aib-Ala-Phe-Lys*3-PyrS2-2F3MeF-BztA-GlnR*3-Ala-NH2 |
| I-199 | Ac-PL3-Asp-Npg-B5-Asp-Asp-Aib-Ala-Phe-Lys*3-PyrS2-2F3MeF-BztA-GlnR*3-Ala-NH2 |
| I-200 | Ac-PL3-Asp-Npg-B5-Asp-Thr-Aib-Ala-Phe-Lys*3-PyrS2-2F3MeF-BztA-GlnR*3-Ala-NH2 |
| I-201 | Ac-PL3-Asp-Npg-B5-Asp-Gln-Aib-Ala-Phe-Lys*3-PyrS2-2F3MeF-BztA-GlnR*3-Ala-NH2 |
| I-202 | Ac-PL3-Asp-Npg-B5-Asp-His-Aib-Ala-Phe-Lys*3-PyrS2-2F3MeF-BztA-GlnR*3-Ala-NH2 |
| I-203 | Ac-PL3-Asp-Npg-B5-Asp-Tyr-Aib-Ala-Phe-Lys*3-PyrS2-2F3MeF-BztA-GlnR*3-Ala-NH2 |
| I-204 | Ac-PL3-Asp-Npg-B5-Asp-5F3Me2COOHF-Aib-Ala-Phe-Lys*3-PyrS2-2F3MeF-BztA-GlnR*3- |
| | Ala-NH2 |
| I-205 | Ac-PL3-Asp-Npg-B5-Asp-4F3Me2COOHF-Aib-Ala-Phe-Lys*3-PyrS2-2F3MeF-BztA-GlnR*3- |
| | Ala-NH2 |
| I-206 | Ac-PL3-Asp-Npg-B5-Asp-5F3Me3COOHF-Aib-Ala-Phe-Lys*3-PyrS2-2F3MeF-BztA-GlnR*3- |
| | Ala-NH2 |
| I-207 | Ac-PL3-Asp-Npg-B5-Asp-4F3Me3COOHF-Aib-Ala-Phe-Lys*3-PyrS2-2F3MeF-BztA-GlnR*3- |
| | Ala-NH2 |
| I-208 | Ac-PL3-Asp-Npg-B5-Asp-3F2COOHF-Aib-Ala-Phe-Lys*3-PyrS2-2F3MeF-BztA-GlnR*3-Ala- |
| | NH2 |
| I-209 | Ac-PL3-Asp-Npg-B5-Asp-Val-Aib-Ala-Phe-Lys*3-PyrS2-2F3MeF-BztA-GlnR*3-Ala-NH2 |
| I-210 | Ac-PL3-Asp-Npg-B5-Asp-Ser-Aib-Ala-Phe-Lys*3-PyrS2-2F3MeF-BztA-GlnR*3-Ala-NH2 |
| I-211 | Ac-PL3-Asp-Npg-B5-Asp-Trp-Aib-Ala-Phe-Lys*3-PyrS2-2F3MeF-BztA-GlnR*3-Ala-NH2 |
| I-212 | Ac-PL3-Asp-Npg-B5-Asp-Asn-Aib-Ala-Phe-Lys*3-PyrS2-2F3MeF-BztA-GlnR*3-Ala-NH2 |
| I-213 | Ac-PL3-Asp-Npg-B5-Asp-Ala-Aib-Ala-Phe-Lys*3-PyrS2-2F3MeF-BztA-GlnR*3-Ala-NH2 |
| I-214 | Ac-PL3-Asp-Npg-B5-Asp-Arg-Aib-Ala-Phe-Lys*3-PyrS2-2F3MeF-BztA-GlnR*3-Ala-NH2 |
| I-215 | Ac-PL3-Asp-Npg-B5-Asp-dGlu-Aib-Ala-Phe-Lys*3-PyrS2-2F3MeF-BztA-GlnR*3-Ala-NH2 |
| I-216 | Ac-PL3-Asp-Npg-B5-Asp-aThr-Aib-Ala-Phe-Lys*3-PyrS2-2F3MeF-BztA-GlnR*3-Ala-NH2 |
| I-217 | Ac-PL3-Asp-Npg-B5-Asp-hTyr-Aib-Ala-Phe-Lys*3-PyrS2-2F3MeF-BztA-GlnR*3-Ala-NH2 |
| I-218 | Ac-PL3-Asp-Npg-B5-Asp-3cbmf-Aib-Ala-Phe-Lys*3-PyrS2-2F3MeF-BztA-GlnR*3-Ala-NH2 |
| I-219 | Ac-PL3-Asp-Npg-B5-Asp-Leu-Aib-Ala-Phe-Lys*3-PyrS2-2F3MeF-BztA-GlnR*3-Ala-NH2 |
| I-220 | Ac-PL3-Asp-Npg-B5-Asp-Phe-Aib-Ala-Phe-Lys*3-PyrS2-2F3MeF-BztA-GlnR*3-Ala-NH2 |
| I-221 | Ac-PL3-Asp-Npg-B5-Asp-Lys-Aib-Ala-Phe-Lys*3-PyrS2-2F3MeF-BztA-GlnR*3-Ala-NH2 |
| I-222 | Ac-PL3-Asp-Npg-B5-Asp-Ile-Aib-Ala-Phe-Lys*3-PyrS2-2F3MeF-BztA-GlnR*3-Ala-NH2 |
| I-223 | Ac-PL3-Asp-Npg-B5-Asp-3COOHF-Aib-Ala-Phe-Lys*3-PyrS2-2F3MeF-BztA-GlnR*3-Ala- |
| | Serol |
| I-224 | Ac-PL3-Asp-Npg-B5-Asp-3COOHF-Aib-Ala-Phe-Lys*3-PyrS2-2F3MeF-BztA-GlnR*3- |
| | MorphNva-Serol |
| I-225 | Ac-PL3-Asp-Npg-B5-Asp-3COOHF-Aib-Ala-Phe-Lys*3-PyrS2-2F3MeF-BztA-GlnR*3- |
| | MorphNva-dAlaol |
| I-226 | Ac-PL3-Asp-Npg-B5-Asp-3COOHF-Aib-Ala-Phe-Lys*3-PyrS2-2F3MeF-BztA-GlnR*3-Ala- |
| | NHEt |
| I-227 | Ac-PL3-Asp-Npg-B5-Asp-3COOHF-Aib-Ala-Phe-Lys*3-PyrS2-2F3MeF-BztA-GlnR*3-Ala-Ser- |
| | NHEt |
| I-228 | Ac-PL3-Asp-Npg-B5-Asp-3COOHF-Aib-Ala-Phe-Lys*3-PyrS2-2F3MeF-BztA-GlnR*3- |
| | MorphNva-NHEt |
| I-229 | Ac-PL3-Asp-Npg-B5-Asp-3COOHF-Aib-Ala-Phe-Lys*3-PyrS2-2F3MeF-BztA-GlnR*3- |
| | MorphNva-Ser-NHEt |
| I-230 | Ac-PL3-Asp-Npg-B5-Asp-3COOHF-Aib-Ala-Phe-Lys*3-PyrS2-2F3MeF-BztA-GlnR*3-Ala-Ser- |
| | NH2 |
| I-231 | Ac-PL3-Asp-Npg-B5-Asp-3COOHF-Aib-Ala-Phe-Lys*3-PyrS2-2F3MeF-BztA-GlnR*3- |
| | MorphNva-NH2 |
| I-232 | Ac-PL3-Asp-Npg-B5-Asp-3COOHF-Aib-Ala-Phe-Lys*3-PyrS2-2F3MeF-BztA-GlnR*3- |
| | MorphNva-Ser-NH2 |
| I-233 | Ac-MePro-Asp-Npg-R4-Asp-3COOHF-Aib-Ala-Phe-Lys*3-PyrS2-3Thi-BztA-GlnR*3-Ala-NH2 |
| I-234 | Ac-MePro-Asp-Npg-R5-Asp-3COOHF-Aib-Ala-Phe-Lys*3-PyrS2-3Thi-BztA-GlnR*3-Ala-NH2 |
| I-235 | Ac-MePro-Asp-Npg-R6-Asp-3COOHF-Aib-Ala-Phe-Lys*3-PyrS2-3Thi-BztA-GlnR*3-Ala-NH2 |
| I-236 | Ac-MePro-Asp-Npg-R5-Asp-3COOHF-Ala-Ala-Phe-Lys*3-PyrS2-3Thi-BztA-GlnR*3-Ala-NH2 |
| I-237 | Ac-MePro-Asp-Val-R5-Asp-3COOHF-Aib-Ala-Phe-Lys*3-PyrS2-3Thi-BztA-GlnR*3-Ala-NH2 |
| I-238 | Ac-MePro-Asp-nLeu-R5-Asp-3COOHF-Aib-Ala-Phe-Lys*3-PyrS2-3Thi-BztA-GlnR*3-Ala-NH2 |
| I-239 | Ac-MePro-Asp-Npg-R4-Asp-TfeGA-Aib-Ala-Phe-Lys*3-PyrS2-3Thi-BztA-GlnR*3-Ala-NH2 |
| I-240 | Ac-MePro-Asp-Npg-R5-Asp-TfeGA-Aib-Ala-Phe-Lys*3-PyrS2-3Thi-BztA-GlnR*3-Ala-NH2 |
| I-241 | Ac-MePro-Asp-Npg-R6-Asp-TfeGA-Aib-Ala-Phe-Lys*3-PyrS2-3Thi-BztA-GlnR*3-Ala-NH2 |
| I-242 | Ac-MePro-Asp-Npg-R5-Asp-TfeGA-Ala-Ala-Phe-Lys*3-PyrS2-3Thi-BztA-GlnR*3-Ala-NH2 |
| I-243 | Ac-MePro-Asp-Val-R5-Asp-TfeGA-Aib-Ala-Phe-Lys*3-PyrS2-3Thi-BztA-GlnR*3-Ala-NH2 |
| I-244 | Ac-MePro-Asp-nLeu-R5-Asp-TfeGA-Aib-Ala-Phe-Lys*3-PyrS2-3Thi-BztA-GlnR*3-Ala-NH2 |
| I-245 | Ac-PL3-Asp-nLeu-B5-Asp-3COOHF-Aib-Ala-Phe-Lys*3-PyrS2-3Thi-BztA-GlnR*3-Ala-NH2 |
| I-246 | Ac-PL3-Asp-Leu-B5-Asp-3COOHF-Aib-Ala-Phe-Lys*3-PyrS2-3Thi-BztA-GlnR*3-Ala-NH2 |
| I-247 | Ac-PL3-Asp-Ile-B5-Asp-3COOHF-Aib-Ala-Phe-Lys*3-PyrS2-3Thi-BztA-GlnR*3-Ala-NH2 |
| I-248 | Ac-PL3-Asp-Val-B5-Asp-3COOHF-Aib-Ala-Phe-Lys*3-PyrS2-3Thi-BztA-GlnR*3-Ala-NH2 |
| I-249 | Ac-PL3-Asp-Phe-B5-Asp-3COOHF-Aib-Ala-Phe-Lys*3-PyrS2-3Thi-BztA-GlnR*3-Ala-NH2 |
| I-250 | Ac-PL3-Asp-Cha-B5-Asp-3COOHF-Aib-Ala-Phe-Lys*3-PyrS2-3Thi-BztA-GlnR*3-Ala-NH2 |
| I-251 | Ac-PL3-Asp-CypA-B5-Asp-3COOHF-Aib-Ala-Phe-Lys*3-PyrS2-3Thi-BztA-GlnR*3-Ala-NH2 |
| I-252 | Ac-PL3-Asp-CyLeu-B5-Asp-3COOHF-Aib-Ala-Phe-Lys*3-PyrS2-3Thi-BztA-GlnR*3-Ala-NH2 |
| I-253 | Ac-PL3-Asp-Chg-B5-Asp-3COOHF-Aib-Ala-Phe-Lys*3-PyrS2-3Thi-BztA-GlnR*3-Ala-NH2 |
| I-254 | Ac-PL3-Asp-Pff-B5-Asp-3COOHF-Aib-Ala-Phe-Lys*3-PyrS2-3Thi-BztA-GlnR*3-Ala-NH2 |
| I-255 | Ac-PL3-Asp-DiethA-B5-Asp-3COOHF-Aib-Ala-Phe-Lys*3-PyrS2-3Thi-BztA-GlnR*3-Ala-NH2 |
| I-256 | Ac-PL3-Asp-Npg-B5-Asp-3COOHF-Ala-Ala-Phe-4PipA*3-PyrS2-3Thi-BztA-GlnR*3-Ala-NH2 |
| I-257 | Ac-PL3-Asp-Npg-B5-Asp-3COOHF-Ala-Ala-Phe-4PipA*3-PyrS2-2F3MeF-BztA-GlnR*3-Ala- |
| | NH2 |
| I-258 | Ac-PL3-Asp-Npg-B5-Asp-3COOHF-Ala-Ala-Phe-GlnR*3-PyrS2-3Thi-BztA-4PipA*3-Ala-NH2 |
| I-259 | Ac-PL3-Asp-Npg-B5-Asp-3COOHF-Ala-Ala-Phe-GlnR*3-PyrS2-2F3MeF-BztA-4PipA*3-Ala- |
| | NH2 |
| I-260 | Ac-PL3-Asp-Npg-B5-Asp-3COOHF-Ala-Ala-Phe-hGlnR*3-PyrS2-3Thi-BztA-4PipA*3-Ala-NH2 |
| I-261 | Ac-PL3-Asp-Npg-B5-Asp-3COOHF-Ala-Ala-Phe-hGlnR*3-PyrS2-2F3MeF-BztA-4PipA*3-Ala- |
| | NH2 |
| I-262 | Ac-PL3-Asp-Npg-B5-Asp-3COOHF-Aib-Ala-Phe-TriAzLys*3-PyrS2-3Thi-BztA-sCH2S*3-Ala- |
| | NH2 |
| I-263 | Ac-PL3-Asp-Npg-B5-Asp-3COOHF-Aib-Ala-Phe-sCH2S*3-PyrS2-3Thi-BztA-TriAzLys*3-Ala- |
| | NH2 |
| I-264 | Ac-PL3-Asp-Npg-B5-Asp-3COOHF-Ala-Ala-Phe-TriAzLys*3-PyrS2-3Thi-BztA-sCH2S*3-Ala- |
| | NH2 |
| I-265 | Ac-PL3-Asp-Npg-B5-Asp-3COOHF-Ala-Ala-Phe-sCH2S*3-PyrS2-3Thi-BztA-TriAzLys*3-Ala- |
| | NH2 |
| I-266 | Ac-PL3-Asp-Npg-B5-Asp-3COOHF-Aib-Ala-Phe-Lys*3-PyrS2-Ala-BztA-GlnR*3-Ala-NH2 |
| I-267 | Ac-PL3-Asp-Npg-B5-Asp-3COOHF-Aib-Ala-Phe-Lys*3-PyrS2-Ala-BztA-GlnR*3-Ala-NH2 |
| I-268 | Ac-PL3-Asp-Npg-B5-Asp-3COOHF-Aib-Ala-Phe-Lys*3-PyrS2-Abu-BztA-GlnR*3-Ala-NH2 |
| I-269 | Ac-PL3-Asp-Npg-B5-Asp-3COOHF-Aib-Ala-Phe-Lys*3-PyrS2-Abu-BztA-GlnR*3-Ala-NH2 |
| I-270 | Ac-PL3-Asp-Npg-B5-Asp-3COOHF-Aib-Ala-Phe-Lys*3-PyrS2-Nva-BztA-GlnR*3-Ala-NH2 |
| I-271 | Ac-PL3-Asp-Npg-B5-Asp-3COOHF-Aib-Ala-Phe-Lys*3-PyrS2-nLeu-BztA-GlnR*3-Ala-NH2 |
| I-272 | Ac-PL3-Asp-Npg-B5-Asp-3COOHF-Aib-Ala-Phe-Lys*3-PyrS2-nLeu-BztA-GlnR*3-Ala-NH2 |
| I-273 | Ac-PL3-Asp-Npg-B5-Asp-3COOHF-Aib-Ala-Phe-Lys*3-PyrS2-Leu-BztA-GlnR*3-Ala-NH2 |
| I-274 | Ac-PL3-Asp-Npg-B5-Asp-3COOHF-Aib-Ala-Phe-Lys*3-PyrS2-Leu-BztA-GlnR*3-Ala-NH2 |
| I-275 | Ac-PL3-Asp-Npg-B5-Asp-3COOHF-Aib-Ala-Phe-Lys*3-PyrS2-hLeu-BztA-GlnR*3-Ala-NH2 |
| I-276 | Ac-PL3-Asp-Npg-B5-Asp-3COOHF-Aib-Ala-Phe-Lys*3-PyrS2-hLeu-BztA-GlnR*3-Ala-NH2 |
| I-277 | Ac-PL3-Asp-Npg-B5-Asp-3COOHF-Aib-Ala-Phe-Lys*3-PyrS2-Npg-BztA-GlnR*3-Ala-NH2 |
| I-278 | Ac-PL3-Asp-Npg-B5-Asp-3COOHF-Aib-Ala-Phe-Lys*3-PyrS2-Npg-BztA-GlnR*3-Ala-NH2 |
| I-279 | Ac-PL3-Asp-Npg-B5-Asp-3COOHF-Aib-Ala-Phe-Lys*3-PyrS2-Phe-BztA-GlnR*3-Ala-NH2 |
| I-280 | Ac-PL3-Asp-Npg-B5-Asp-3COOHF-Aib-Ala-Phe-Lys*3-PyrS2-Phe-BztA-GlnR*3-Ala-NH2 |
| I-281 | Ac-PL3-Asp-Npg-B5-Asp-3COOHF-Aib-Ala-Phe-Lys*3-PyrS2-Cpa-BztA-GlnR*3-Ala-NH2 |
| I-282 | Ac-PL3-Asp-Npg-B5-Asp-3COOHF-Aib-Ala-Phe-Lys*3-PyrS2-Cpa-BztA-GlnR*3-Ala-NH2 |
| I-283 | Ac-PL3-Asp-Npg-B5-Asp-3COOHF-Aib-Ala-Phe-Lys*3-PyrS2-Cba-BztA-GlnR*3-Ala-NH2 |
| I-284 | Ac-PL3-Asp-Npg-B5-Asp-3COOHF-Aib-Ala-Phe-Lys*3-PyrS2-CypA-BztA-GlnR*3-Ala-NH2 |
| I-285 | Ac-PL3-Asp-Npg-B5-Asp-3COOHF-Aib-Ala-Phe-Lys*3-PyrS2-ChA-BztA-GlnR*3-Ala-NH2 |
| I-286 | Ac-PL3-Asp-Npg-B5-SbMeAsp-3COOHF-Aib-Ala-Phe-Lys*3-PyrS2-2F3MeF-BztA-GlnR*3- |
| | Ala-NH2 |
| I-287 | Ac-PL3-Asp-Npg-B5-RbMeAsp-3COOHF-Aib-Ala-Phe-Lys*3-PyrS2-2F3MeF-BztA-GlnR*3- |
| | Ala-NH2 |
| I-288 | Ac-PL3-SbMeAsp-Npg-B5-Asp-3COOHF-Aib-Ala-Phe-Lys*3-PyrS2-2F3MeF-BztA-GlnR*3- |
| | Ala-NH2 |
| I-289 | Ac-PL3-RbMeAsp-Npg-B5-Asp-3COOHF-Aib-Ala-Phe-Lys*3-PyrS2-2F3MeF-BztA-GlnR*3- |
| | Ala-NH2 |
| I-290 | Ac-PL3-aMeDAsp-Npg-B5-Asp-3COOHF-Aib-Ala-Phe-Lys*3-PyrS2-2F3MeF-BztA-GlnR*3- |
| | Ala-NH2 |
| I-291 | Ac-PL3-Glu-Npg-B5-Glu-3COOHF-Aib-Ala-Phe-Lys*3-PyrS2-2F3MeF-BztA-GlnR*3-Ala-NH2 |
| I-292 | Ac-PL3-Asp-Npg-B5-Asp-3COOHF-Aib-Ala-Phe-Lys*3-PyrS2-2FurA-BztA-GlnR*3-Ala-NH2 |
| I-293 | Ac-PL3-Asp-Npg-B5-Asp-3COOHF-Aib-Ala-Phe-Lys*3-PyrS2-20MeF-BztA-GlnR*3-Ala-NH2 |
| I-294 | Ac-PL3-Asp-Npg-B5-Asp-3COOHF-Aib-Ala-Phe-Lys*3-PyrS2-2MeF-BztA-GlnR*3-Ala-NH2 |
| I-295 | Ac-PL3-Asp-Npg-B5-Asp-3COOHF-Aib-Ala-Phe-Lys*3-PyrS2-2BrF-BztA-GlnR*3-Ala-NH2 |
| I-296 | Ac-PL3-Asp-Npg-B5-Asp-3COOHF-Aib-Ala-Phe-Lys*3-PyrS2-2ClF-BztA-GlnR*3-Ala-NH2 |
| I-297 | Ac-PL3-Asp-Npg-B5-Asp-3COOHF-Aib-Ala-Phe-Lys*3-PyrS2-2CNF-BztA-GlnR*3-Ala-NH2 |
| I-298 | Ac-PL3-Asp-Npg-B5-Asp-3COOHF-Aib-Ala-Phe-Lys*3-PyrS2-2NO2F-BztA-GlnR*3-Ala-NH2 |
| I-299 | Ac-PL3-Asp-Npg-B5-Asp-3COOHF-Aib-Ala-Phe-Lys*3-PyrS2-2PyrA-BztA-GlnR*3-Ala-NH2 |
| I-300 | Ac-PL3-Asp-Npg-B5-Asp-3COOHF-Aib-Ala-Phe-Lys*3-PyrS2-3PyrA-BztA-GlnR*3-Ala-NH2 |
| I-301 | Ac-PL3-Asp-Npg-B5-Asp-3COOHF-Aib-Ala-Phe-Lys*3-PyrS2-4PyrA-BztA-GlnR*3-Ala-NH2 |
| I-302 | Ac-PL3-Asp-Npg-B5-Asp-3COOHF-Aib-Ala-Phe-Lys*3-PyrS2-His-BztA-GlnR*3-Ala-NH2 |
| I-303 | Ac-PL3-Asp-Npg-B5-Asp-3COOHF-Aib-Ala-Phe-Lys*3-PyrS2-2F3MeF-BztA-GlnR*3- |
| | [BiotinPEG8]Lys-NH2 |
| I-304 | Ac-PL3-Asp-Ala-B5-Asp-3COOHF-Aib-Ala-Phe-Lys*3-PyrS2-3Thi-BztA-GlnR*3-Ala-NH2 |
| I-305 | Ac-PL3-Asp-Tyr-B5-Asp-3COOHF-Aib-Ala-Phe-Lys*3-PyrS2-3Thi-BztA-GlnR*3-Ala-NH2 |
| I-306 | Ac-PL3-Asp-Trp-B5-Asp-3COOHF-Aib-Ala-Phe-Lys*3-PyrS2-3Thi-BztA-GlnR*3-Ala-NH2 |
| I-307 | Ac-PL3-Asp-Ser-B5-Asp-3COOHF-Aib-Ala-Phe-Lys*3-PyrS2-3Thi-BztA-GlnR*3-Ala-NH2 |
| I-308 | Ac-PL3-Asp-Aib-B5-Asp-3COOHF-Aib-Ala-Phe-Lys*3-PyrS2-3Thi-BztA-GlnR*3-Ala-NH2 |
| I-309 | Ac-PL3-Asp-Phg-B5-Asp-3COOHF-Aib-Ala-Phe-Lys*3-PyrS2-3Thi-BztA-GlnR*3-Ala-NH2 |
| I-310 | Ac-PL3-Asp-DipA-B5-Asp-3COOHF-Aib-Ala-Phe-Lys*3-PyrS2-3Thi-BztA-GlnR*3-Ala-NH2 |
| I-311 | Ac-PL3-Asp-OctG-B5-Asp-3COOHF-Aib-Ala-Phe-Lys*3-PyrS2-3Thi-BztA-GlnR*3-Ala-NH2 |
| I-312 | Ac-PL3-Asp-Cba-B5-Asp-3COOHF-Aib-Ala-Phe-Lys*3-PyrS2-3Thi-BztA-GlnR*3-Ala-NH2 |
| I-313 | Ac-PL3-Asp-MorphNva-B5-Asp-3COOHF-Aib-Ala-Phe-Lys*3-PyrS2-3Thi-BztA-GlnR*3-Ala- |
| | NH2 |
| I-314 | Ac-PL3-Asp-F2PipNva-B5-Asp-3COOHF-Aib-Ala-Phe-Lys*3-PyrS2-3Thi-BztA-GlnR*3-Ala- |
| | NH2 |
| I-315 | Ac-PL3-Asn-Npg-B5-Asp-[CH2CMe2CO2H]TriAzDap-Aib-Ala-Phe-Lys*3-PyrS2-3Thi-BztA- |
| | GlnR*3-Ala-NH2 |
| I-316 | Ac-PL3-Ser-Npg-B5-Asp-[CH2CMe2CO2H]TriAzDap-Aib-Ala-Phe-Lys*3-PyrS2-3Thi-BztA- |
| | GlnR*3-Ala-NH2 |
| I-317 | Ac-PL3-aThr-Npg-B5-Asp-[CH2CMe2CO2H]TriAzDap-Aib-Ala-Phe-Lys*3-PyrS2-3Thi-BztA- |
| | GlnR*3-Ala-NH2 |
| I-318 | Ac-PL3-Asp-Npg-B5-Asn-[CH2CMe2CO2H]TriAzDap-Aib-Ala-Phe-Lys*3-PyrS2-3Thi-BztA- |
| | GlnR*3-Ala-NH2 |
| I-319 | Ac-PL3-Asn-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-320 | Ac-PL3-Ser-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-321 | Ac-PL3-aThr-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-322 | Ac-PL3-Asp-Npg-B5-Asn-[CH2CMe2CO2H]TriAzDap-MorphGln-Ala-Phe-Lys*3-PyrS2-3Thi- |
| | BztA-GlnR*3-Ala-NH2 |
| I-323 | Ac-PL3-Asn-Npg-B5-Asp-3COOHF-MorphGln-Ala-Phe-Lys*3-PyrS2-3Thi-BztA-GlnR*3-Ala- |
| | NH2 |
| I-324 | Ac-PL3-Ser-Npg-B5-Asp-3COOHF-MorphGln-Ala-Phe-Lys*3-PyrS2-3Thi-BztA-GlnR*3-Ala- |
| | NH2 |
| I-325 | Ac-PL3-aThr-Npg-B5-Asp-3COOHF-MorphGln-Ala-Phe-Lys*3-PyrS2-3Thi-BztA-GlnR*3-Ala- |
| | NH2 |
| I-326 | Ac-PL3-Asp-Npg-B5-Asn-3COOHF-MorphGln-Ala-Phe-Lys*3-PyrS2-3Thi-BztA-GlnR*3-Ala- |
| | NH2 |
| I-327 | Ac-PL3-Asp-Npg-B5-Asp-3COOHF-Aib-Ala-Phe-Lys*3-PyrS2-2F3MeF-34ClF-GlnR*3-Ala- |
| | NH2 |
| I-328 | Ac-PL3-Asp-Npg-B5-Asp-3COOHF-Aib-Ala-Phe-Lys*3-PyrS2-3Thi-34ClF-GlnR*3-Ala-NH2 |
| I-329 | Ac-PL3-Asp-Npg-B5-Asp-3COOHF-Aib-Ala-Phe-Lys*3-PyrS2-Phe-34ClF-GlnR*3-Ala-NH2 |
| I-330 | Ac-PL3-Asp-Npg-B5-Asp-3COOHF-Aib-Ala-Phe-Lys*3-PyrS2-2F3MeF-34MeF-GlnR*3-Ala- |
| | NH2 |
| I-331 | Ac-PL3-Asp-Npg-B5-Asp-3COOHF-Aib-Ala-Phe-Lys*3-PyrS2-2F3MeF-3BrF-GlnR*3-Ala-NH2 |
| I-332 | Ac-PL3-Asp-Npg-B5-Asp-3COOHF-Aib-Ala-Phe-Lys*3-PyrS2-3Thi-3BrF-GlnR*3-Ala-NH2 |
| I-333 | Ac-PL3-Asp-Npg-B5-Asp-3COOHF-Aib-Ala-Phe-Lys*3-PyrS2-Phe-3BrF-GlnR*3-Ala-NH2 |
| I-334 | Ac-PL3-Asp-Npg-B5-Asp-3COOHF-Aib-Ala-Phe-Lys*3-PyrS2-1NapA-BztA-GlnR*3-Ala-NH2 |
| I-335 | Ac-PL3-Asp-Npg-B5-Asp-3COOHF-Aib-Ala-Phe-TriAzLys*3-PyrS2-3Thi-BztA-sAla*3-Ala- |
| | NH2 |
| I-336 | Ac-PL3-Asp-Npg-B5-Asp-3COOHF-Aib-Ala-Phe-sAla*3-PyrS2-3Thi-BztA-TriAzLys*3-Ala- |
| | NH2 |
| I-337 | Ac-PL3-Asp-Npg-B5-Asp-3COOHF-Ala-Ala-Phe-TriAzLys*3-PyrS2-3Thi-BztA-sAla*3-Ala- |
| | NH2 |
| I-338 | Ac-PL3-Asp-Npg-B5-Asp-3COOHF-Ala-Ala-Phe-sAla*3-PyrS2-3Thi-BztA-TriAzLys*3-Ala- |
| | NH2 |
| I-339 | Ac-PL3-Asp-Npg-B5-Asp-3COOHF-Aib-Ala-Phe-[4FB]Cys-PyrS2-2F3MeF-BztA-Cys-Ala-NH2 |
| I-340 | Ac-PL3-Asp-Npg-B5-Asp-3COOHF-Aib-Ala-Phe-[8FBB]Cys-PyrS2-2F3MeF-BztA-Cys-Ala- |
| | NH2 |
| I-341 | Ac-PL3-Asp-Npg-B5-Asp-3COOHF-Aib-Ala-Phe-[8FBB]Cys-PyrS2-2F3MeF-BztA-Cys-Ala- |
| | NH2 |
| I-342 | Ac-PL3-Asp-Npg-B5-Asp-3COOHF-Aib-Ala-Phe-Lys*3-PyrS2-3Thi-BztA-GlnR*3-Glu-Ala- |
| | NH2 |
| I-343 | Ac-PL3-Asp-Npg-B5-Asp-3COOHF-Aib-Ala-Phe-Lys*3-PyrS2-3Thi-BztA-GlnR*3-Ala-Glu- |
| | Ala-NH2 |
| I-344 | Ac-PL3-Asp-Npg-B5-Asp-3COOHF-Aib-Ala-Phe-Lys*3-PyrS2-2COOHF-BztA-GlnR*3-Ala- |
| | NH2 |
| I-345 | Ac-PL3-Asp-Npg-B5-Asp-3COOHF-Aib-Ala-3COOHF-Lys*3-PyrS2-3Thi-BztA-GlnR*3-Ala- |
| | NH2 |
| I-346 | Ac-PL3-Asp-Npg-B5-Asp-3COOHF-Aib-Ala-3COOHF-Lys*3-PyrS2-2COOHF-BztA-GlnR*3- |
| | Ala-NH2 |
| I-347 | Ac-PL3-Asp-Npg-B5-Asp-3COOHF-Aib-Ala-3COOHF-Lys*3-PyrS2-2COOHF-BztA-GlnR*3- |
| | Ala-Glu-Ala-NH2 |
| I-348 | Ac-PL3-Asp-Npg-B5-Asp-3COOHF-Aib-Ala-Phe-Lys*3-PyrS2-3Thi-BztA-GlnR*3-Glu-Ala-OH |
| I-349 | Ac-PL3-Asp-Npg-B5-Asp-3COOHF-Aib-Ala-Phe-Lys*3-PyrS2-2COOHF-BztA-GlnR*3-Ala-OH |
| I-350 | Ac-PL3-Asp-Npg-B5-Asp-3COOHF-Aib-Ala-3COOHF-Lys*3-PyrS2-3Thi-BztA-GlnR*3-Ala- |
| | OH |
| I-351 | Ac-PL3-Asp-Npg-B5-Asp-3COOHF-Aib-Ala-3COOHF-Lys*3-PyrS2-2COOHF-BztA-GlnR*3- |
| | Ala-OH |
| I-352 | Ac-PL3-Asp-Npg-B5-Asp-3COOHF-Aib-Ala-3COOHF-Lys*3-PyrS2-2COOHF-BztA-GlnR*3- |
| | Ala-Glu-Ala-OH |
| I-353 | Ac-PL3-Asp-Npg-B5-Asp-3COOHF-Aib-Ala-Cba-Lys*3-PyrS2-3Thi-BztA-GlnR*3-Ala-NH2 |
| I-354 | Ac-PL3-Asp-Npg-B5-Asp-3COOHF-Aib-Ala-CypA-Lys*3-PyrS2-3Thi-BztA-GlnR*3-Ala-NH2 |
| I-355 | Ac-PL3-Asp-Npg-B5-Asp-3COOHF-Aib-Ala-BztA-Lys*3-PyrS2-3Thi-BztA-GlnR*3-Ala-NH2 |
| I-356 | Ac-PL3-Asp-Npg-B5-Asp-3COOHF-Aib-Ala-1NapA-Lys*3-PyrS2-3Thi-BztA-GlnR*3-Ala-NH2 |
| I-357 | Ac-PL3-Asp-Npg-B5-Asp-3COOHF-Aib-Ala-2NapA-Lys*3-PyrS2-3Thi-BztA-GlnR*3-Ala-NH2 |
| I-358 | Ac-PL3-Asp-Npg-B5-Asp-3COOHF-Aib-Ala-Tyr-Lys*3-PyrS2-3Thi-BztA-GlnR*3-Ala-NH2 |
| I-359 | Ac-PL3-Asp-Npg-B5-Asp-3COOHF-Aib-Ala-Trp-Lys*3-PyrS2-3Thi-BztA-GlnR*3-Ala-NH2 |
| I-360 | Ac-PL3-Asp-Npg-B5-Asp-3COOHF-Aib-Ala-Leu-Lys*3-PyrS2-3Thi-BztA-GlnR*3-Ala-NH2 |
| I-361 | Ac-PL3-Asp-Npg-B5-Asp-3COOHF-Aib-Ala-Ile-Lys*3-PyrS2-3Thi-BztA-GlnR*3-Ala-NH2 |
| I-362 | Ac-PL3-Asp-Npg-B5-Asp-3COOHF-Aib-Ala-nLeu-Lys*3-PyrS2-3Thi-BztA-GlnR*3-Ala-NH2 |
| I-363 | Ac-PL3-Asp-Npg-B5-Asp-3COOHF-Aib-Ala-Ser-Lys*3-PyrS2-3Thi-BztA-GlnR*3-Ala-NH2 |
| I-364 | Ac-PL3-Asp-Npg-B5-Asp-3COOHF-Aib-Ala-3Thi-Lys*3-PyrS2-3Thi-BztA-GlnR*3-Ala-NH2 |
| I-365 | Ac-PL3-Asp-Npg-B5-Asp-3COOHF-Aib-Ala-2Thi-Lys*3-PyrS2-3Thi-BztA-GlnR*3-Ala-NH2 |
| I-366 | Ac-PL3-Asp-Npg-B5-Asp-3COOHF-Aib-Ala-Chg-Lys*3-PyrS2-3Thi-BztA-GlnR*3-Ala-NH2 |
| I-367 | Ac-PL3-Asp-Npg-B5-Asp-3COOHF-Aib-Ala-Hse-Lys*3-PyrS2-3Thi-BztA-GlnR*3-Ala-NH2 |
| I-368 | Ac-PL3-Asp-Npg-B5-Asp-3COOHF-Aib-Ala-4TriA-Lys*3-PyrS2-3Thi-BztA-GlnR*3-Ala-NH2 |
| I-369 | Ac-PL3-Asp-Npg-B5-Asp-3COOHF-Aib-Ala-3F3MeF-Lys*3-PyrS2-3Thi-BztA-GlnR*3-Ala- |
| | NH2 |
| I-370 | Ac-PL3-Asp-Npg-B5-Asp-3COOHF-Aib-Ala-Thr-Lys*3-PyrS2-3Thi-BztA-GlnR*3-Ala-NH2 |
| I-371 | Ac-PL3-Asp-Npg-B5-Asp-3COOHF-Aib-Ala-His-Lys*3-PyrS2-3Thi-BztA-GlnR*3-Ala-NH2 |
| I-372 | Ac-PL3-Asp-Npg-B5-Asp-3COOHF-Aib-Ala-Val-Lys*3-PyrS2-3Thi-BztA-GlnR*3-Ala-NH2 |
| I-373 | Ac-PL3-Asp-Npg-B5-Asp-3COOHF-Aib-Ala-Asn-Lys*3-PyrS2-3Thi-BztA-GlnR*3-Ala-NH2 |
| I-374 | Ac-PL3-Asp-Npg-B5-Asp-3COOHF-Aib-Ala-Gln-Lys*3-PyrS2-3Thi-BztA-GlnR*3-Ala-NH2 |
| I-375 | Ac-PL3-Asp-Npg-B5-Asp-3COOHF-Aib-Ala-Phe-[mXyl]Cys-PyrS2-2F3MeF-BztA-Cys-Ala- |
| | NH2 |
| I-376 | Ac-PL3-Asp-Npg-B5-Asp-3COOHF-Aib-Ala-Phe-[oXyl]Cys-PyrS2-2F3MeF-BztA-Cys-Ala- |
| | NH2 |
| I-377 | Ac-PL3-Asp-Npg-B5-Asp-3COOHF-Aib-Ala-Phe-[pXyl]Cys-PyrS2-2F3MeF-BztA-Cys-Ala- |
| | NH2 |
| I-378 | Ac-PL3-Asp-Npg-B5-Asp-3COOHF-MorphGln-Ala-Phe-Lys*3-PyrS2-3Thi-BztA-GlnR*3-Ala- |
| | NH2 |
| I-379 | Ac-PL3-Asp-Npg-B5-Asp-3COOHF-Me2Gln-Ala-Phe-Lys*3-PyrS2-3Thi-BztA-GlnR*3-Ala- |
| | NH2 |
| I-380 | Ac-PL3-Asp-Npg-B5-Asp-3COOHF-Met20-Ala-Phe-Lys*3-PyrS2-3Thi-BztA-GlnR*3-Ala-NH2 |
| I-381 | Ac-PL3-Asp-Npg-B5-Asp-3COOHF-AcLys-Ala-Phe-Lys*3-PyrS2-3Thi-BztA-GlnR*3-Ala-NH2 |
| I-382 | Ac-PL3-Asp-Npg-B5-Asp-3COOHF-CyLeu-Ala-Phe-Lys*3-PyrS2-3Thi-BztA-GlnR*3-Ala-NH2 |
| I-383 | Ac-PL3-Asp-Npg-B5-Asp-3COOHF-CyLeu-Ala-Phe-Lys*3-PyrS2-3Thi-BztA-GlnR*3-Ala-NH2 |
| I-384 | Ac-PL3-Asp-Npg-B5-Asp-3COOHF-Acp-Ala-Phe-Lys*3-PyrS2-3Thi-BztA-GlnR*3-Ala-NH2 |
| I-385 | Ac-PL3-Asp-Npg-B5-Asp-3COOHF-Acp-Ala-Phe-Lys*3-PyrS2-3Thi-BztA-GlnR*3-Ala-NH2 |
| I-386 | Ac-PL3-Asp-Npg-B5-Asp-3COOHF-Phe-Ala-Phe-Lys*3-PyrS2-3Thi-BztA-GlnR*3-Ala-NH2 |
| I-387 | Ac-PL3-Asp-Npg-B5-Asp-3COOHF-His-Ala-Phe-Lys*3-PyrS2-3Thi-BztA-GlnR*3-Ala-NH2 |
| I-388 | Ac-PL3-Asp-Npg-B5-Asp-3COOHF-Val-Ala-Phe-Lys*3-PyrS2-3Thi-BztA-GlnR*3-Ala-NH2 |
| I-389 | Ac-PL3-Asp-Npg-B5-Asp-3COOHF-Ser-Ala-Phe-Lys*3-PyrS2-3Thi-BztA-GlnR*3-Ala-NH2 |
| I-390 | Ac-PL3-Asp-Npg-B5-Asp-3COOHF-Ser-Ala-Phe-Lys*3-PyrS2-3Thi-BztA-GlnR*3-Ala-NH2 |
| I-391 | Ac-PL3-Asp-Npg-B5-Asp-3COOHF-Gln-Ala-Phe-Lys*3-PyrS2-3Thi-BztA-GlnR*3-Ala-NH2 |
| I-392 | Ac-PL3-Asp-Npg-B5-Asp-3COOHF-Gln-Ala-Phe-Lys*3-PyrS2-3Thi-BztA-GlnR*3-Ala-NH2 |
| I-393 | Ac-PL3-Asp-Npg-B5-Asp-3COOHF-Asn-Ala-Phe-Lys*3-PyrS2-3Thi-BztA-GlnR*3-Ala-NH2 |
| I-394 | Ac-PL3-Asp-Npg-B5-Asp-3COOHF-Asn-Ala-Phe-Lys*3-PyrS2-3Thi-BztA-GlnR*3-Ala-NH2 |
| I-395 | Ac-PL3-Asp-Npg-B5-Asp-3COOHF-Aib-Ala-2Cpg-Lys*3-PyrS2-3Thi-BztA-GlnR*3-Ala-NH2 |
| I-396 | Ac-PL3-Asp-Npg-B5-Asp-3COOHF-Aib-Ala-Phe-Lys*3-PyrS2-3Thi-BztA-GlnR*3-Val-NH2 |
| I-397 | Ac-PL3-Asp-Npg-B5-Asp-3COOHF-Aib-Ala-Phe-Lys*3-PyrS2-3Thi-BztA-GlnR*3-Aib-NH2 |
| I-398 | Ac-PL3-Asp-Npg-B5-Asp-3COOHF-Aib-Ala-Phe-Lys*3-PyrS2-3Thi-BztA-GlnR*3-Thr-NH2 |
| I-399 | Ac-PL3-Asp-Npg-B5-Asp-3COOHF-Aib-Ala-Phe-Lys*3-PyrS2-3Thi-BztA-GlnR*3-Val-Ser- |
| | NH2 |
| I-400 | Ac-PL3-Asp-Npg-B5-Asp-3COOHF-Aib-Ala-Phe-Lys*3-PyrS2-3Thi-BztA-GlnR*3-Aib-Ser- |
| | NH2 |
| I-401 | Ac-PL3-Asp-Npg-B5-Asp-3COOHF-Aib-Ala-Phe-Lys*3-PyrS2-3Thi-BztA-GlnR*3-Thr-Ser- |
| | NH2 |
| I-402 | Ac-PL3-Asp-Npg-B5-Asp-3COOHF-Aib-Ala-Phe-Lys*3-PyrS2-3Thi-BztA-GlnR*3-Val-Ala- |
| | NH2 |
| I-403 | Ac-PL3-Asp-Npg-B5-Asp-3COOHF-Aib-Ala-Phe-Lys*3-PyrS2-3Thi-BztA-GlnR*3-Val-Ala- |
| | NH2 |
| I-404 | Ac-PL3-Asp-Npg-B5-Asp-3COOHF-Aib-Ala-Phe-Lys*3-PyrS2-3Thi-BztA-GlnR*3-Aib-Ala- |
| | NH2 |
| I-405 | Ac-PL3-Asp-Npg-B5-Asp-3COOHF-Aib-Ala-Phe-Lys*3-PyrS2-3Thi-BztA-GlnR*3-Aib-Ala- |
| | NH2 |
| I-406 | Ac-PL3-Asp-Npg-B5-Asp-3COOHF-Aib-Ala-Phe-Lys*3-PyrS2-3Thi-BztA-GlnR*3-Thr-Ala- |
| | NH2 |
| I-407 | Ac-PL3-Asp-Npg-B5-Asp-3COOHF-Aib-Ala-Phe-Lys*3-PyrS2-3Thi-BztA-GlnR*3-Val-Aib- |
| | NH2 |
| I-408 | Ac-PL3-Asp-Npg-B5-Asp-3COOHF-Aib-Ala-Phe-Lys*3-PyrS2-3Thi-BztA-GlnR*3-Val-Aib- |
| | NH2 |
| I-409 | Ac-PL3-Asp-Npg-B5-Asp-3COOHF-Aib-Ala-Phe-Lys*3-PyrS2-3Thi-BztA-GlnR*3-Aib-Aib- |
| | NH2 |
| I-410 | Ac-PL3-Asp-Npg-B5-Asp-3COOHF-Aib-Ala-Phe-Lys*3-PyrS2-3Thi-BztA-GlnR*3-Aib-Aib- |
| | NH2 |
| I-411 | Ac-PL3-Asp-Npg-B5-Asp-3COOHF-Aib-Ala-Phe-Lys*3-PyrS2-3Thi-BztA-GlnR*3-Thr-Aib- |
| | NH2 |
| I-412 | Ac-PL3-Asp-Npg-B5-Asp-3COOHF-Aib-Ala-Phe-Lys*3-PyrS2-3Thi-BztA-GlnR*3-Thr-Aib- |
| | NH2 |
| I-413 | Ac-PL3-Asp-Npg-B5-Asp-3COOHF-Aib-Ala-Phe-TriAzLys*3-PyrS2-3Thi-BztA-sAla*3-Val- |
| | NH2 |
| I-414 | Ac-PL3-Asp-Npg-B5-Asp-3COOHF-Aib-Ala-Phe-TriAzLys*3-PyrS2-3Thi-BztA-sAla*3-Leu- |
| | NH2 |
| I-415 | Ac-PL3-Asp-Npg-B5-Asp-3COOHF-Aib-Ala-Phe-TriAzLys*3-PyrS2-3Thi-BztA-sAla*3-Thr- |
| | NH2 |
| I-416 | Ac-PL3-Asp-Npg-B5-Asp-3COOHF-Aib-Ala-Phe-TriAzLys*3-PyrS2-3Thi-BztA-sAla*3-Ala- |
| | Ser-NH2 |
| I-417 | Ac-PL3-Asp-Npg-B5-Asp-3COOHF-Aib-Ala-Phe-TriAzLys*3-PyrS2-3Thi-BztA-sAla*3-Val- |
| | Ser-NH2 |
| I-418 | Ac-PL3-Asp-Npg-B5-Asp-3COOHF-Aib-Ala-Phe-TriAzLys*3-PyrS2-Phe-BztA-sAla*3-Ala- |
| | NH2 |
| I-419 | Ac-PL3-Asp-Npg-B5-Asp-3COOHF-Aib-Ala-Phe-TriAzLys*3-PyrS2-Phe-BztA-sAbu*3-Ala- |
| | NH2 |
| I-420 | Ac-PL3-Asp-Npg-B5-Asp-3COOHF-Aib-Ala-Phe-TriAzLys*3-PyrS2-Phe-BztA-sAbu*3-Ala- |
| | NH2 |
| I-421 | Ac-PL3-Asp-Npg-B5-Asp-3COOHF-Aib-Ala-Phe-TriAzOrn*3-PyrS2-3Thi-BztA-sAla*3-Ala- |
| | NH2 |
| I-422 | Ac-PL3-Asp-Npg-B5-Asp-3COOHF-Aib-Ala-Phe-TriAzOrn*3-PyrS2-Phe-BztA-sAla*3-Ala- |
| | NH2 |
| I-423 | Ac-PL3-Asp-Npg-B5-Asp-3COOHF-Aib-Ala-Phe-TriAzOrn*3-PyrS2-3Thi-BztA-sAbu*3-Ala- |
| | NH2 |
| I-424 | Ac-PL3-Asp-Npg-B5-Asp-3COOHF-Aib-Ala-Phe-TriAzOrn*3-PyrS2-Phe-BztA-sAbu*3-Ala- |
| | NH2 |
| I-425 | 4penteny1-MePro-Asp-AllylGly-R5-Asp-3COOHF-Aib-Ala-Phe-Lys*3-PyrS2-3Thi-BztA- |
| | GlnR*3-Ala-NH2 |
| I-426 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-427 | 4penteny1-MePro-Asp-AllylGly-R5-Asp-3COOHF-Aib-Ala-Phe-Lys*3-PyrS2-3Thi-BztA- |
| | GlnR*3-Ala-NH2 |
| I-428 | 4pentenyl-MePro-Asp-AllylGly-R5-Asp-3COOHF-Aib-Ala-Phe-Lys*3-PyrS2-3Thi-BztA- |
| | GlnR*3-Ala-NH2 |
| I-429 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-430 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-431 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-432 | Ac-PL3-Asp-Npg-B5-Asp-3COOHF-aMeL-Ala-Phe-Lys*3-PyrS2-3Thi-BztA-GlnR*3-Ala-NH2 |
| I-433 | Ac-PL3-Asp-Npg-B5-Asp-3COOHF-DaMeL-Ala-Phe-Lys*3-PyrS2-3Thi-BztA-GlnR*3-Ala- |
| | NH2 |
| I-434 | Ac-PL3-Asp-Npg-B5-Asp-3COOHF-aMeV-Ala-Phe-Lys*3-PyrS2-3Thi-BztA-GlnR*3-Ala-NH2 |
| I-435 | Ac-PL3-Asp-Npg-B5-Asp-3COOHF-aMeS-Ala-Phe-Lys*3-PyrS2-3Thi-BztA-GlnR*3-Ala-NH2 |
| I-436 | Ac-PL3-Asp-Npg-B5-Asp-3COOHF-DaMeS-Ala-Phe-Lys*3-PyrS2-3Thi-BztA-GlnR*3-Ala-NH2 |
| I-437 | Ac-PL3-Asp-Npg-B5-Asp-3COOHF-DaMeS-Ala-Phe-Lys*3-PyrS2-3Thi-BztA-GlnR*3-Ala-NH2 |
| I-438 | Ac-PL3-Asp-Npg-B5-Asp-3COOHF-aMeF-Ala-Phe-Lys*3-PyrS2-3Thi-BztA-GlnR*3-Ala-NH2 |
| I-439 | Ac-PL3-Asp-Npg-B5-Asp-3COOHF-Aib-Aib-Phe-Lys*3-PyrS2-3Thi-BztA-GlnR*3-Ala-NH2 |
| I-440 | Ac-PL3-Asp-Npg-B5-Asp-3COOHF-Aib-Cpg-Phe-Lys*3-PyrS2-3Thi-BztA-GlnR*3-Ala-NH2 |
| I-441 | Ac-PL3-Asp-Npg-B5-Asp-3COOHF-Ala-Aib-Phe-Lys*3-PyrS2-3Thi-BztA-GlnR*3-Ala-NH2 |
| I-442 | Ac-PL3-Asp-Npg-B5-Asp-3COOHF-Ala-Aib-Phe-Lys*3-PyrS2-3Thi-BztA-GlnR*3-Ala-NH2 |
| I-443 | Ac-PL3-Asp-Npg-B5-Asp-3COOHF-Ala-Cpg-Phe-Lys*3-PyrS2-3Thi-BztA-GlnR*3-Ala-NH2 |
| I-444 | Ac-PL3-Asp-Npg-B5-Asp-3COOHF-Aib-Ser-Phe-Lys*3-PyrS2-3Thi-BztA-GlnR*3-Ala-NH2 |
| I-445 | Ac-PL3-Asp-Npg-B5-Asp-3COOHF-Aib-Thr-Phe-Lys*3-PyrS2-3Thi-BztA-GlnR*3-Ala-NH2 |
| I-446 | Ac-PL3-Asp-Npg-B5-Asp-3COOHF-Aib-aThr-Phe-Lys*3-PyrS2-3Thi-BztA-GlnR*3-Ala-NH2 |
| I-447 | Ac-PL3-Asp-Npg-B5-Asp-3COOHF-Aib-Val-Phe-Lys*3-PyrS2-3Thi-BztA-GlnR*3-Ala-NH2 |
| I-448 | Ac-PL3-Asp-Npg-B5-Asp-3COOHF-Aib-Val-Phe-Lys*3-PyrS2-3Thi-BztA-GlnR*3-Ala-NH2 |
| I-449 | Ac-PL3-Asp-Npg-B5-Asp-3COOHF-Aib-Leu-Phe-Lys*3-PyrS2-3Thi-BztA-GlnR*3-Ala-NH2 |
| I-450 | Ac-PL3-Asp-Npg-B5-Asp-3COOHF-Aib-Leu-Phe-Lys*3-PyrS2-3Thi-BztA-GlnR*3-Ala-NH2 |
| I-451 | Ac-PL3-Asp-Npg-B5-Asp-3COOHF-Aib-Gln-Phe-Lys*3-PyrS2-3Thi-BztA-GlnR*3-Ala-NH2 |
| I-452 | Ac-PL3-Asp-Npg-B5-Asp-3COOHF-Aib-Gln-Phe-Lys*3-PyrS2-3Thi-BztA-GlnR*3-Ala-NH2 |
| I-453 | Ac-PL3-Asp-Npg-B5-Asp-3COOHF-Aib-MorphGln-Phe-Lys*3-PyrS2-3Thi-BztA-GlnR*3-Ala- |
| | NH2 |
| I-454 | Ac-PL3-Asp-Npg-B5-Asp-3COOHF-Aib-Lys-Phe-Lys*3-PyrS2-3Thi-BztA-GlnR*3-Ala-NH2 |
| I-455 | Ac-PL3-Asp-Npg-B5-Asp-3COOHF-Aib-Lys-Phe-Lys*3-PyrS2-3Thi-BztA-GlnR*3-Ala-NH2 |
| I-456 | Ac-PL3-Asp-Npg-B5-Asp-3COOHF-aMeDF-Ala-Phe-Lys*3-PyrS2-3Thi-BztA-GlnR*3-Ala-NH2 |
| I-457 | Ac-PL3-Asp-Npg-B5-Asp-3COOHF-aMeDF-Ala-Phe-Lys*3-PyrS2-3Thi-BztA-GlnR*3-Ala-NH2 |
| I-458 | Ac-PL3-Asp-Npg-B5-Asp-3COOHF-Aib-Ala-Phe-Lys*3-PyrS2-3Thi-BztA-GlnR*3-dAla-NH2 |
| I-459 | Ac-PL3-Asp-Npg-B5-Asp-3COOHF-Aib-Ala-Phe-Lys*3-PyrS2-3Thi-BztA-GlnR*3-dAla-NH2 |
| I-460 | Ac-PL3-Asp-Npg-B5-Asp-3COOHF-Aib-Ala-Phe-Lys*3-PyrS2-3Thi-BztA-GlnR*3-dAla-Ser- |
| | NH2 |
| I-461 | Ac-PL3-Asp-Npg-B5-Asp-3COOHF-Aib-Ala-Phe-Lys*3-PyrS2-3Thi-BztA-GlnR*3-dAla-Ser- |
| | NH2 |
| I-462 | Ac-PL3-Asp-Npg-B5-Asp-3COOHF-Aib-Ala-Phe-Lys*3-PyrS2-3Thi-BztA-GlnR*3-dLeu-NH2 |
| I-463 | Ac-PL3-Asp-Npg-B5-Asp-3COOHF-Aib-Ala-Phe-Lys*3-PyrS2-3Thi-BztA-GlnR*3-dLeu-Ser- |
| | NH2 |
| I-464 | Ac-PL3-Asp-Npg-B5-Asp-3COOHF-Aib-Ala-Phe-Lys*3-PyrS2-3Thi-BztA-GlnR*3-Ala-Ser- |
| | Leu-NH2 |
| I-465 | Ac-PL3-Asp-Npg-B5-Asp-3COOHF-Aib-Ala-Phe-Lys*3-PyrS2-3Thi-BztA-GlnR*3-Leu-Ser- |
| | Leu-NH2 |
| I-466 | Ac-PL3-Asp-Npg-B5-Asp-3COOHF-Aib-Ala-Phe-Lys*3-PyrS2-3Thi-BztA-GlnR*3-Leu-Ser- |
| | Leu-NH2 |
| I-467 | Ac-PL3-Asp-Npg-B5-Asp-3COOHF-Aib-Ala-Phe-Lys*3-PyrS2-3Thi-BztA-GlnR*3-dLeu-Ser- |
| | Leu-NH2 |
| I-468 | Ac-PL3-Asp-Npg-B5-Asp-3COOHF-Aib-Ala-Phe-Lys*3-PyrS2-3Thi-BztA-GlnR*3-dLeu-Ser- |
| | Leu-NH2 |
| I-469 | Ac-PL3-OAsp-Npg-B5-Asp-3COOHF-Aib-Ala-Phe-Lys*3-PyrS2-2F3MeF-BztA-GlnR*3-Ala- |
| | NH2 |
| I-470 | Ac-PL3-Glu-Npg-B5-Glu-3COOHF-Aib-Ala-Phe-Lys*3-PyrS2-3Thi-BztA-GlnR*3-Ala-NH2 |
| I-471 | BzAm20Allyl-MePro-Asp-AllylGly-R5-Asp-3COOHF-Aib-Ala-Phe-Lys*3-PyrS2-3Thi-BztA- |
| | GlnR*3-Ala-NH2 |
| I-472 | Ac-PL3-Asp-Npg-B5-Asp-3COOHF-Aib-Ala-Phe-Lys*3-PyrS2-2ClF-34ClF-GlnR*3-Ala-NH2 |
| I-473 | Ac-PL3-Asp-Npg-B5-Asp-3COOHF-Aib-Ala-Phe-Lys*3-PyrS2-nLeu-34ClF-GlnR*3-Ala-NH2 |
| I-474 | Ac-PL3-Asp-Npg-B5-Asp-3COOHF-Aib-Ala-Phe-Lys*3-PyrS2-nLeu-34ClF-GlnR*3-Ala-NH2 |
| I-475 | Ac-PL3-Asp-Npg-B5-Asp-3COOHF-Aib-Ala-Phe-Lys*3-PyrS2-CypA-34ClF-GlnR*3-Ala-NH2 |
| I-476 | Ac-PL3-Asp-Npg-B5-Asp-3COOHF-Aib-Ala-Phe-Lys*3-PyrS2-CypA-34ClF-GlnR*3-Ala-NH2 |
| I-477 | Ac-PL3-Asp-Npg-B5-Asp-3COOHF-Aib-Ala-Tyr-Lys*3-PyrS2-Phe-34ClF-GlnR*3-Ala-NH2 |
| I-478 | Ac-PL3-Asp-Npg-B5-Asp-3COOHF-Aib-Ala-3Thi-Lys*3-PyrS2-Phe-34ClF-GlnR*3-Ala-NH2 |
| I-479 | Ac-PL3-Asp-Npg-B5-Asp-3COOHF-Aib-Ala-nLeu-Lys*3-PyrS2-Phe-34ClF-GlnR*3-Ala-NH2 |
| I-480 | Ac-PL3-Asp-Npg-B5-Asp-3COOHF-Aib-Ala-nLeu-Lys*3-PyrS2-Phe-34ClF-GlnR*3-Ala-NH2 |
| I-481 | Ac-PL3-Asp-Npg-B5-Asp-3COOHF-Aib-Ala-2NapA-Lys*3-PyrS2-Phe-34ClF-GlnR*3-Ala-NH2 |
| I-482 | Ac-PL3-Asp-Npg-B5-Asp-3COOHF-Aib-Ala-2NapA-Lys*3-PyrS2-Phe-34ClF-GlnR*3-Ala-NH2 |
| I-483 | Ac-PL3-Asp-Npg-B5-Asp-3COOHF-Aib-Ala-Phe-Lys*3-PyrS2-Phe-2NapA-GlnR*3-Ala-NH2 |
| I-484 | Ac-PL3-Asp-Npg-B5-Asp-3COOHF-Aib-Ala-Phe-Lys*3-PyrS2-Phe-2NapA-GlnR*3-Ala-NH2 |
| I-485 | Ac-PL3-Asp-Npg-B5-Asp-3COOHF-Aib-Ala-Phe-Lys*3-PyrS2-3Thi-2NapA-GlnR*3-Ala-NH2 |
| I-486 | Ac-PL3-Asp-Npg-B5-Asp-3COOHF-Aib-Ala-Phe-Lys*3-PyrS2-3Thi-2NapA-GlnR*3-Ala-NH2 |
| I-487 | Ac-PL3-Asp-Npg-B5-Asp-3COOHF-Aib-Ala-Phe-Lys*3-PyrS2-2ClF-2NapA-GlnR*3-Ala-NH2 |
| I-488 | Ac-PL3-Asp-Npg-B5-Asp-3COOHF-Aib-Ala-Phe-Lys*3-PyrS2-2ClF-2NapA-GlnR*3-Ala-NH2 |
| I-489 | Ac-PL3-Asp-Npg-B5-Asp-3COOHF-Aib-Ala-Phe-Lys*3-PyrS2-nLeu-2NapA-GlnR*3-Ala-NH2 |
| I-490 | Ac-PL3-Asp-Npg-B5-Asp-3COOHF-Aib-Ala-Phe-Lys*3-PyrS2-nLeu-2NapA-GlnR*3-Ala-NH2 |
| I-491 | Ac-PL3-Asp-Npg-B5-Asp-3COOHF-Aib-Ala-Phe-Lys*3-PyrS2-CypA-2NapA-GlnR*3-Ala-NH2 |
| I-492 | Ac-PL3-Asp-Npg-B5-Asp-3COOHF-Aib-Ala-Phe-Lys*3-PyrS2-CypA-2NapA-GlnR*3-Ala-NH2 |
| I-493 | Ac-PL3-Asp-Npg-B5-Asp-3COOHF-Aib-Ala-Phe-Lys*3-PyrS2-3Thi-BztA-GlnR*3-Leu-Asp- |
| | Ala-NH2 |
| I-494 | Ac-PL3-Asp-Npg-B5-Asp-3COOHF-Aib-Ala-Phe-Lys*3-PyrS2-3Thi-BztA-GlnR*3-Leu-Glu- |
| | Ala-NH2 |
| I-495 | Ac-PL3-Asp-Npg-B5-Asp-3COOHF-Aib-Ala-Phe-Lys*3-PyrS2-3Thi-BztA-GlnR*3-Leu-Glu- |
| | Ala-NH2 |
| I-496 | Ac-PL3-Asp-Npg-B5-Asp-3COOHF-Aib-Ala-Phe-Lys*3-PyrS2-3Thi-BztA-GlnR*3-Leu-Ser- |
| | Ala-NH2 |
| I-497 | Ac-PL3-Asp-Npg-B5-Asp-3COOHF-Aib-Ala-Phe-Lys*3-PyrS2-3Thi-BztA-GlnR*3-Leu-Ser- |
| | Ala-NH2 |
| I-498 | Ac-PL3-Asp-Npg-B5-Asp-3COOHF-Aib-Ala-Phe-Lys*3-PyrS2-3Thi-BztA-GlnR*3-Leu-Thr- |
| | Ala-NH2 |
| I-499 | Ac-PL3-Asp-Npg-B5-Asp-3COOHF-Aib-Ala-Phe-Lys*3-PyrS2-3Thi-BztA-GlnR*3-Leu-Thr- |
| | Ala-NH2 |
| I-500 | Ac-PL3-Asp-Npg-B5-Asp-3COOHF-Aib-Ala-Phe-Lys*3-PyrS2-3Thi-BztA-GlnR*3-Leu-aThr- |
| | Ala-NH2 |
| I-501 | Ac-PL3-Asp-Npg-B5-Asp-3COOHF-Aib-Ala-Phe-Lys*3-PyrS2-3Thi-BztA-GlnR*3-Leu-aThr- |
| | Ala-NH2 |
| I-502 | Ac-PL3-Asp-Npg-B5-Asp-3COOHF-Aib-Ala-Phe-Lys*3-PyrS2-3Thi-BztA-GlnR*3-Leu-Asp- |
| | Leu-NH2 |
| I-503 | Ac-PL3-Asp-Npg-B5-Asp-3COOHF-Aib-Ala-Phe-Lys*3-PyrS2-3Thi-BztA-GlnR*3-Leu-Asp- |
| | Leu-NH2 |
| I-504 | Ac-PL3-Asp-Npg-B5-Asp-3COOHF-Aib-Ala-Phe-Lys*3-PyrS2-3Thi-BztA-GlnR*3-Leu-Glu- |
| | Leu-NH2 |
| I-505 | Ac-PL3-Asp-Npg-B5-Asp-3COOHF-Aib-Ala-Phe-Lys*3-PyrS2-3Thi-BztA-GlnR*3-Leu-Glu- |
| | Leu-NH2 |
| I-506 | Ac-PL3-Asp-Npg-B5-Asp-3COOHF-Aib-Ala-Phe-Lys*3-PyrS2-3Thi-BztA-GlnR*3-Val-Asp- |
| | Ala-NH2 |
| I-507 | Ac-PL3-Asp-Npg-B5-Asp-3COOHF-Aib-Ala-Phe-Lys*3-PyrS2-3Thi-BztA-GlnR*3-Val-Glu- |
| | Ala-NH2 |
| I-508 | Ac-PL3-Asp-Npg-B5-Asp-3COOHF-Aib-Ala-Phe-Lys*3-PyrS2-3Thi-BztA-GlnR*3-Val-Thr- |
| | Ala-NH2 |
| I-509 | Ac-PL3-Asp-Npg-B5-Asp-3COOHF-Aib-Ala-Phe-Lys*3-PyrS2-3Thi-BztA-GlnR*3-Val-Thr- |
| | Ala-NH2 |
| I-510 | Ac-PL3-Asp-Npg-B5-Asp-3COOHF-Aib-Ala-Phe-Lys*3-PyrS2-3Thi-BztA-GlnR*3-Val-aThr- |
| | Ala-NH2 |
| I-511 | Ac-PL3-Asp-Npg-B5-Asp-3COOHF-Aib-Ala-Phe-Lys*3-PyrS2-3Thi-BztA-GlnR*3-Val-aThr- |
| | Ala-NH2 |
| I-512 | Ac-PL3-Asp-Npg-B5-Asp-3COOHF-Aib-Ala-Phe-Lys*3-PyrS2-3Thi-BztA-GlnR*3-Val-Asp- |
| | Leu-NH2 |
| I-513 | Ac-PL3-Asp-Npg-B5-Asp-3COOHF-Aib-Ala-Phe-Lys*3-PyrS2-3Thi-BztA-GlnR*3-Val-Asp- |
| | Leu-NH2 |
| I-514 | Ac-Gly-PL3-Asp-Npg-B5-Asp-3COOHF-Aib-Ala-Phe-Lys*3-PyrS2-3Thi-BztA-GlnR*3-Ala |
| I-515 | Ac-Sar-PL3-Asp-Npg-B5-Asp-3COOHF-Aib-Ala-Phe-Lys*3-PyrS2-3Thi-BztA-GlnR*3-Ala- |
| | NH2 |
| I-516 | Ac-NMebAla-PL3-Asp-Npg-B5-Asp-3COOHF-Aib-Ala-Phe-Lys*3-PyrS2-3Thi-BztA-GlnR*3- |
| | Ala-NH2 |
| I-517 | Ac-PL3-Asp-Npg-B5-Asp-3COOHF-Aib-Ala-Phe-Lys*3-PyrS2-3Thi-BztA-GlnR*3- |
| | [BiotinPEG8]Lys-NH2 |
| I-518 | Ac-PL3-Glu-Npg-B5-Glu-3COOHF-Aib-Ala-Phe-Lys*3-PyrS2-3Thi-BztA-GlnR*3- |
| | [BiotinPEG8]Lys-NH2 |
| I-519 | 5hexenyl-MePro-Asp-[Bn][Allyl]Dap-R5-Asp-3COOHF-Aib-Ala-Phe-Lys*3-PyrS2-3Thi-BztA- |
| | GlnR*3-Ala-NH2 |
| I-520 | 5hexenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-521 | Ac-PL3-Asp-Npg-B5-Asp-3COOHF-Aib-Asp-Phe-Lys*3-PyrS2-3Thi-BztA-GlnR*3-Ala-NH2 |
| I-522 | Ac-PL3-Asp-Npg-B5-aThr-3COOHF-Aib-Asp-Phe-Lys*3-PyrS2-3Thi-BztA-GlnR*3-Ala-NH2 |
| I-523 | Ac-PL3-Asp-Npg-B5-Asp-3COOHF-Aib-Glu-Phe-Lys*3-PyrS2-3Thi-BztA-GlnR*3-Ala-NH2 |
| I-524 | Ac-PL3-Asp-Npg-B5-aThr-3COOHF-Aib-Glu-Phe-Lys*3-PyrS2-3Thi-BztA-GlnR*3-Ala-NH2 |
| I-525 | Ac-PL3-Asp-Npg-B5-Asp-3COOHF-Aib-Aad-Phe-Lys*3-PyrS2-3Thi-BztA-GlnR*3-Ala-NH2 |
| I-526 | Ac-PL3-Asp-Npg-B5-aThr-3COOHF-Aib-Aad-Phe-Lys*3-PyrS2-3Thi-BztA-GlnR*3-Ala-NH2 |
| I-527 | Ac-PL3-Asp-Npg-B5-Asp-3COOHF-Aib-nLeu-Phe-Lys*3-PyrS2-3Thi-BztA-GlnR*3-Ala-NH2 |
| I-528 | Ac-PL3-Asp-Npg-B5-Asp-3COOHF-Aib-nLeu-Phe-Lys*3-PyrS2-3Thi-BztA-GlnR*3-Ala-NH2 |
| I-529 | Ac-PL3-Asp-Npg-B5-Asp-3COOHF-Aib-Phe-Phe-Lys*3-PyrS2-3Thi-BztA-GlnR*3-Ala-NH2 |
| I-530 | Ac-PL3-Asp-Npg-B5-Asp-3COOHF-Aib-hPhe-Phe-Lys*3-PyrS2-3Thi-BztA-GlnR*3-Ala-NH2 |
| I-531 | Ac-PL3-Asp-Npg-B5-Asp-3COOHF-Aib-Cba-Phe-Lys*3-PyrS2-3Thi-BztA-GlnR*3-Ala-NH2 |
| I-532 | Ac-PL3-Asp-Npg-B5-Asp-3COOHF-Aib-Cba-Phe-Lys*3-PyrS2-3Thi-BztA-GlnR*3-Ala-NH2 |
| I-533 | Ac-PL3-Asp-Npg-B5-Asp-3COOHF-Aib-hTyr-Phe-Lys*3-PyrS2-3Thi-BztA-GlnR*3-Ala-NH2 |
| I-534 | Ac-PL3-Asp-Npg-B5-Asp-3COOHF-Aib-AcLys-Phe-Lys*3-PyrS2-3Thi-BztA-GlnR*3-Ala-NH2 |
| I-535 | Ac-PL3-Asp-Npg-B5-Asp-3COOHF-Aib-Ala-Phe-Lys*3-PyrS2-Val-BztA-GlnR*3-Ala-NH2 |
| I-536 | Ac-PL3-Asp-Npg-B5-Asp-3COOHF-Aib-Ala-Phe-Lys*3-PyrS2-Ile-BztA-GlnR*3-Ala-NH2 |
| I-537 | Ac-PL3-Asp-Npg-B5-Asp-3COOHF-Aib-Ala-Phe-Lys*3-PyrS2-Chg-BztA-GlnR*3-Ala-NH2 |
| I-538 | Ac-PL3-Asp-Npg-B5-Asp-3COOHF-Aib-Ala-Phe-Lys*3-PyrS2-DiethA-BztA-GlnR*3-Ala-NH2 |
| I-539 | Ac-PL3-Asp-Npg-B5-Asp-3COOHF-Aib-Ala-Phe-Lys*3-PyrS2-hnLeu-BztA-GlnR*3-Ala-NH2 |
| I-540 | Ac-PL3-Asp-Npg-B5-Asp-3COOHF-Aib-Ala-Phe-Lys*3-PyrS2-OctG-BztA-GlnR*3-Ala-NH2 |
| I-541 | Ac-PL3-Asp-Npg-B5-Asp-3COOHF-Aib-Ala-Phe-Lys*3-PyrS2-2Thi-BztA-GlnR*3-Ala-NH2 |
| I-542 | Ac-PL3-Asp-Npg-B5-Asp-3COOHF-Aib-Ala-Phe-Lys*3-PyrS2-2cbmF-BztA-GlnR*3-Ala-NH2 |
| I-543 | Ac-PL3-Glu-Npg-B5-Asp-3COOHF-Aib-Ala-Phe-Lys*3-PyrS2-3Thi-BztA-GlnR*3-Ala-NH2 |
| I-544 | Ac-PL3-Glu-Npg-B5-Asp-3COOHF-Aib-Ala-Phe-Lys*3-PyrS2-3Thi-BztA-GlnR*3-Ala-NH2 |
| I-545 | Ac-PL3-Aad-Npg-B5-Asp-3COOHF-Aib-Ala-Phe-Lys*3-PyrS2-3Thi-BztA-GlnR*3-Ala-NH2 |
| I-546 | Ac-PL3-Aad-Npg-B5-Asp-3COOHF-Aib-Ala-Phe-Lys*3-PyrS2-3Thi-BztA-GlnR*3-Ala-NH2 |
| I-547 | Ac-PL3-Asp-Npg-B5-Glu-3COOHF-Aib-Ala-Phe-Lys*3-PyrS2-3Thi-BztA-GlnR*3-Ala-NH2 |
| I-548 | Ac-PL3-Asp-Npg-B5-Aad-3COOHF-Aib-Ala-Phe-Lys*3-PyrS2-3Thi-BztA-GlnR*3-Ala-NH2 |
| I-549 | Ac-PL3-Aad-Npg-B5-Glu-3COOHF-Aib-Ala-Phe-Lys*3-PyrS2-3Thi-BztA-GlnR*3-Ala-NH2 |
| I-550 | Ac-PL3-Glu-Npg-B5-Aad-3COOHF-Aib-Ala-Phe-Lys*3-PyrS2-3Thi-BztA-GlnR*3-Ala-NH2 |
| I-551 | Ac-PL3-Aad-Npg-B5-Aad-3COOHF-Aib-Ala-Phe-Lys*3-PyrS2-3Thi-BztA-GlnR*3-Ala-NH2 |
| I-552 | Ac-PL3-Glu-Npg-B5-Glu-Glu-Aib-Ala-Phe-Lys*3-PyrS2-3Thi-BztA-GlnR*3-Ala-NH2 |
| I-553 | Ac-PL3-Aad-Npg-B5-Glu-Glu-Aib-Ala-Phe-Lys*3-PyrS2-3Thi-BztA-GlnR*3-Ala-NH2 |
| I-554 | Ac-PL3-Glu-Npg-B5-Glu-Asp-Aib-Ala-Phe-Lys*3-PyrS2-3Thi-BztA-GlnR*3-Ala-NH2 |
| I-555 | Ac-PL3-Aad-Npg-B5-Glu-Asp-Aib-Ala-Phe-Lys*3-PyrS2-3Thi-BztA-GlnR*3-Ala-NH2 |
| I-556 | Ac-PL3-Aad-Npg-B5-Aad-Glu-Aib-Ala-Phe-Lys*3-PyrS2-3Thi-BztA-GlnR*3-Ala-NH2 |
| I-557 | Ac-PL3-Asp-Npg-B5-Asp-3COOHF-Aib-Ala-Phe-dLys*3-PyrS2-3Thi-BztA-dGlnR*3-Ala-NH2 |
| I-558 | Ac-PL3-Asp-Npg-B5-Asp-3COOHF-Aib-Ala-Phe-dOrn*3-PyrS2-3Thi-BztA-GlnR*3-Ala-NH2 |
| I-559 | Ac-PL3-Asp-Npg-B5-Asp-3COOHF-Aib-Ala-Phe-dDab*3-PyrS2-3Thi-BztA-GlnR*3-Ala-NH2 |
| I-560 | Ac-PL3-Asp-Npg-B5-Asp-3COOHF-Aib-Ala-Phe-Orn*3-PyrS2-3Thi-BztA-hGlnR*3-Ala-NH2 |
| I-561 | Ac-PL3-Asp-Npg-B5-Asp-3COOHF-Aib-Ala-Phe-NMeOrn*3-PyrS2-3Thi-BztA-hGlnR*3-Ala- |
| | NH2 |
| I-562 | 4pentenyl-MePro-Asp-[Bn][Allyl]Dap-R5-Asp-3COOHF-Aib-Ala-Phe-Lys*3-PyrS2-3Thi-BztA- |
| | GlnR*3-Ala-NH2 |
| I-563 | 4pentenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-564 | 5hexenyl-MePro-Asp-[Piv][Allyl]Dap-R5-Asp-3COOHF-Aib-Ala-Phe-Lys*3-PyrS2-3Thi-BztA- |
| | GlnR*3-Ala-NH2 |
| I-565 | 5hexenyl-MePro-Asp-[CyCO][Allyl]Dap-R5-Asp-3COOHF-Aib-Ala-Phe-Lys*3-PyrS2-3Thi- |
| | BztA-GlnR*3-Ala-NH2 |
| I-566 | Ac-PL3-Asp-Npg-B5-Asp-3COOHF-Aib-Ala-Phe-[2_6-naph]Cys-PyrS2-3Thi-BztA-Cys-Ala- |
| | NH2 |
| I-567 | Ac-PL3-Asp-Npg-B5-Asp-3COOHF-Aib-Ala-Phe-[3_3-biph]Cys-PyrS2-3Thi-BztA-Cys-Ala- |
| | NH2 |
| I-568 | Ac-PL3-Asp-Npg-B5-Asp-3COOHF-Aib-Ala-4FF-Lys*3-PyrS2-3Thi-BztA-GlnR*3-Ala-NH2 |
| I-569 | Ac-PL3-Asp-Npg-B5-Asp-3COOHF-Aib-Ala-4FF-Lys*3-PyrS2-3Thi-BztA-GlnR*3-Ala-NH2 |
| I-570 | Ac-PL3-Asp-Npg-B5-Asp-3COOHF-Aib-Ala-4ClF-Lys*3-PyrS2-3Thi-BztA-GlnR*3-Ala-NH2 |
| I-571 | Ac-PL3-Asp-Npg-B5-Asp-3COOHF-Aib-Ala-4ClF-Lys*3-PyrS2-3Thi-BztA-GlnR*3-Ala-NH2 |
| I-572 | Ac-PL3-Asp-Npg-B5-Asp-3COOHF-Aib-Ala-4BrF-Lys*3-PyrS2-3Thi-BztA-GlnR*3-Ala-NH2 |
| I-573 | Ac-PL3-Asp-Npg-B5-Asp-3COOHF-Aib-Ala-4BrF-Lys*3-PyrS2-3Thi-BztA-GlnR*3-Ala-NH2 |
| I-574 | Ac-PL3-Asp-Npg-B5-Asp-3COOHF-Aib-Ala-3FF-Lys*3-PyrS2-3Thi-BztA-GlnR*3-Ala-NH2 |
| I-575 | Ac-PL3-Asp-Npg-B5-Asp-3COOHF-Aib-Ala-3FF-Lys*3-PyrS2-3Thi-BztA-GlnR*3-Ala-NH2 |
| I-576 | Ac-PL3-Asp-Npg-B5-Asp-3COOHF-Aib-Ala-3ClF-Lys*3-PyrS2-3Thi-BztA-GlnR*3-Ala-NH2 |
| I-577 | Ac-PL3-Asp-Npg-B5-Asp-3COOHF-Aib-Ala-3ClF-Lys*3-PyrS2-3Thi-BztA-GlnR*3-Ala-NH2 |
| I-578 | Ac-PL3-Asp-Npg-B5-Asp-3COOHF-Aib-Ala-3BrF-Lys*3-PyrS2-3Thi-BztA-GlnR*3-Ala-NH2 |
| I-579 | Ac-PL3-Asp-Npg-B5-Asp-3COOHF-Aib-Ala-3BrF-Lys*3-PyrS2-3Thi-BztA-GlnR*3-Ala-NH2 |
| I-580 | Ac-PL3-Asp-Npg-B5-Asp-3COOHF-Aib-Ala-2FF-Lys*3-PyrS2-3Thi-BztA-GlnR*3-Ala-NH2 |
| I-581 | Ac-PL3-Asp-Npg-B5-Asp-3COOHF-Aib-Ala-2FF-Lys*3-PyrS2-3Thi-BztA-GlnR*3-Ala-NH2 |
| I-582 | Ac-PL3-Asp-Npg-B5-Asp-3COOHF-Aib-Ala-30MeF-Lys*3-PyrS2-3Thi-BztA-GlnR*3-Ala- |
| | NH2 |
| I-583 | Ac-PL3-Asp-Npg-B5-Asp-3COOHF-Aib-Ala-30MeF-Lys*3-PyrS2-3Thi-BztA-GlnR*3-Ala- |
| | NH2 |
| I-584 | Ac-PL3-Asp-Npg-B5-Asp-3COOHF-Aib-Ala-4CNF-Lys*3-PyrS2-3Thi-BztA-GlnR*3-Ala-NH2 |
| I-585 | Ac-PL3-Asp-Npg-B5-Asp-3COOHF-Aib-Ala-4CNF-Lys*3-PyrS2-3Thi-BztA-GlnR*3-Ala-NH2 |
| I-586 | Ac-PL3-Asp-Npg-B5-Asp-3COOHF-Aib-Ala-3CNF-Lys*3-PyrS2-3Thi-BztA-GlnR*3-Ala-NH2 |
| I-587 | Ac-PL3-Asp-Npg-B5-Asp-3COOHF-Aib-Ala-3CNF-Lys*3-PyrS2-3Thi-BztA-GlnR*3-Ala-NH2 |
| I-588 | Ac-PL3-Asp-Npg-B5-Asp-3COOHF-Aib-Ala-4MeF-Lys*3-PyrS2-3Thi-BztA-GlnR*3-Ala-NH2 |
| I-589 | Ac-PL3-Asp-Npg-B5-Asp-3COOHF-Aib-Ala-4MeF-Lys*3-PyrS2-3Thi-BztA-GlnR*3-Ala-NH2 |
| I-590 | Ac-PL3-Asp-Npg-B5-Asp-3COOHF-Aib-Ala-3MeF-Lys*3-PyrS2-3Thi-BztA-GlnR*3-Ala-NH2 |
| I-591 | Ac-PL3-Asp-Npg-B5-Asp-3COOHF-Aib-Ala-3MeF-Lys*3-PyrS2-3Thi-BztA-GlnR*3-Ala-NH2 |
| I-592 | Ac-PL3-Asp-Npg-B5-Asp-3COOHF-Aib-Ala-Aic-Lys*3-PyrS2-3Thi-BztA-GlnR*3-Ala-NH2 |
| I-593 | Ac-PL3-Asp-Npg-B5-Asp-3COOHF-Aib-Ala-Aic-Lys*3-PyrS2-3Thi-BztA-GlnR*3-Ala-NH2 |
| I-594 | Ac-PL3-Asp-Npg-B5-Asp-3COOHF-Aib-Ala-RbiPrF-Lys*3-PyrS2-3Thi-BztA-GlnR*3-Ala-NH2 |
| I-595 | Ac-PL3-Asp-Npg-B5-Asp-3COOHF-Aib-Ala-RbiPrF-Lys*3-PyrS2-3Thi-BztA-GlnR*3-Ala-NH2 |
| I-596 | Ac-PL3-Asp-Npg-B5-Asp-3COOHF-Aib-Ala-SbiPrF-Lys*3-PyrS2-3Thi-BztA-GlnR*3-Ala-NH2 |
| I-597 | Ac-PL3-Asp-Npg-B5-Asp-3COOHF-Aib-Ala-SbiPrF-Lys*3-PyrS2-3Thi-BztA-GlnR*3-Ala-NH2 |
| I-598 | Ac-PL3-Asp-Npg-B5-Asp-3COOHF-Aib-Ala-RbiPrDF-Lys*3-PyrS2-3Thi-BztA-GlnR*3-Ala- |
| | NH2 |
| I-599 | Ac-PL3-Asp-Npg-B5-Asp-3COOHF-Aib-Ala-RbiPrDF-Lys*3-PyrS2-3Thi-BztA-GlnR*3-Ala- |
| | NH2 |
| I-600 | Ac-PL3-Asp-Npg-B5-Asp-3COOHF-Aib-Ala-RbMeXylA-Lys*3-PyrS2-3Thi-BztA-GlnR*3-Ala- |
| | NH2 |
| I-601 | Ac-PL3-Asp-Npg-B5-Asp-3COOHF-Aib-Ala-RbMeXylA-Lys*3-PyrS2-3Thi-BztA-GlnR*3-Ala- |
| | NH2 |
| I-602 | Ac-PL3-Asp-Npg-B5-Asp-3COOHF-Aib-Ala-RbMeXylDA-Lys*3-PyrS2-3Thi-BztA-GlnR*3- |
| | Ala-NH2 |
| I-603 | Ac-PL3-Asp-Npg-B5-Asp-3COOHF-Aib-Ala-RbMeXylDA-Lys*3-PyrS2-3Thi-BztA-GlnR*3- |
| | Ala-NH2 |
| I-604 | Ac-PL3-Asp-Npg-B5-Asp-3COOHF-Aib-Ala-SbMeXylA-Lys*3-PyrS2-3Thi-BztA-GlnR*3-Ala- |
| | NH2 |
| I-605 | Ac-PL3-Asp-Npg-B5-Asp-3COOHF-Aib-Ala-SbMeXylDA-Lys*3-PyrS2-3Thi-BztA-GlnR*3- |
| | Ala-NH2 |
| I-606 | Ac-PL3-Glu-Npg-B5-Glu-3COOHF-Aib-Ala-Phe-Lys*3-PyrS2-3Thi-BztA-GlnR*3-AzLys-NH2 |
| I-607 | Ac-PL3-Asp-Npg-B5-Asp-3COOHF-Aib-Ala-Phe-Lys*3-PyrS2-3Thi-BztA-GlnR*3-AzLys-NH2 |

Certain results from various additional assessment for various additional agents and compositions are presented in Table E3 below. See Table E1 and Table E2 for description. Among other things, these data confirm that technologies of the present disclosure can provide various activities and/or benefits.

TABLE E3
Certain data of various compositions as examples.

| 1 | 2 | 3 | 4 | 5 | 6 | 7 | 8 | 9 |
|---|---|---|---|---|---|---|---|---|
| I-608 | A | | | | 1956.8426 | | | |
| I-609 | A | | | | 1956.8426 | | | |
| I-700 | A | | | | 1956.8426 | | | |
| I-701 | B | | | | 2136.9519 | | | |
| I-702 | B | | | | 2164.9832 | 2166.5 | 2164.6 | |
| I-703 | B | | | | 2150.9675 | 2152.6 | 2150.6 | |
| I-704 | A | C | | | 2178.9988 | 2180.3 | 2178.4 | |
| I-705 | A | C | | | 2221.0458 | 2222.4 | 2220.5 | |
| I-706 | A | | | | 2060.9052 | | | |
| I-707 | A | | | | 2086.9209 | | | |
| I-708 | A | B | | | 2170.9209 | | | |
| I-709 | A | A | | + | 2084.9165 | | | |
| I-710 | A | B | | | 2102.9522 | 2104.9 | 2102.9 | |
| I-711 | A | B | | | 2088.9365 | 2090.8 | 2089.1 | |
| I-712 | A | A | | | 2003.8838 | 2005.7 | 2003.8 | |
| I-713 | A | | | | 2003.8838 | 2005.7 | 2003.9 | |
| I-714 | A | A | | | 2060.9052 | 2062.8 | 2061 | |
| I-715 | A | A | | | 2116.9678 | 2118.8 | 2116.9 | |
| I-716 | A | B | | | 2116.9678 | 2118.9 | 2116.9 | |
| I-717 | A | B | | | 2130.9835 | 2132.9 | 2131.2 | |
| I-718 | A | C | | | 2130.9835 | 2132.8 | 2131 | |
| I-719 | A | A | ++ | + | 2100.9365 | 2102.8 | 2100.4 | |
| I-720 | A | C | ++ | + | 2100.9365 | 2102.8 | 2100.7 | |
| I-721 | A | B | ++ | + | 2090.9158 | 2092.8 | 2090.9 | |
| I-722 | A | B | + | | 2150.9522 | 2152.9 | 2151 | |
| I-723 | A | B | | | 2117.9267 | 2119.7 | 2117.8 | |
| I-724 | A | B | | | 2131.9424 | 2133.8 | 2132.2 | |
| I-725 | A | C | | | 2189.9631 | 2191.9 | 2189.7 | |
| I-726 | B | C | | | 2189.9631 | 2191.9 | 2190.2 | |
| I-727 | A | B | | | 2166.9471 | 2168.9 | 2166.3 | |
| I-728 | A | B | | | 2166.9471 | 2169 | 2166.7 | |
| I-729 | A | A | | | 2122.9209 | 2124.7 | 2122.7 | |
| I-730 | A | | | | 2086.9209 | 2088.7 | 2086.7 | |
| I-731 | A | | | | 2128.9678 | 2130.8 | 2128.9 | |
| I-732 | A | A | | | 2134.9209 | 2136.7 | 2134.4 | |
| I-733 | A | A | | | 2098.9209 | 2100.8 | 2098.9 | |
| I-734 | A | B | | | 2140.9678 | 2142.8 | 2140.6 | |
| I-735 | A | C | | | 2148.9365 | 2150.8 | 2149.1 | |
| I-736 | A | A | | | 2112.9365 | 2114.8 | 2113 | |
| I-737 | A | A | | | 2154.9835 | 2156.8 | 2154.6 | |
| I-738 | A | C | | | 2232.9365 | | | |
| I-739 | A | B | | | 2232.9365 | 2234.8 | 2232.8 | |
| I-740 | A | A | | | 2196.9365 | 2199 | 2197 | |
| I-741 | E | D | | +++ | 2074.9209 | 2076.8 | 2075 | |
| I-742 | E | D | | +++ | 2074.9209 | 2076.8 | 2075 | |
| I-743 | C | D | | | 2101.8954 | 2103 | 2100.8 | |
| I-744 | A | D | | | 2115.9111 | 2117.9 | 2115.7 | |
| I-745 | A | C | | | 2115.9111 | 2117.9 | 2115.9 | |
| I-746 | A | C | | | 2130.9835 | 2132.8 | 2130.9 | |
| I-747 | A | B | | + | 2102.9522 | 2104.8 | 2103 | |
| I-748 | A | C | | + | 2138.927 | 2140.8 | 2138.6 | |
| I-749 | A | C | | | 2164.9678 | 2166.9 | 2164.9 | |
| I-750 | A | B | | | 2118.9471 | 2120.8 | 2118.3 | |
| I-751 | C | D | | | 2391.1975 | 2393.7 | 2391.6 | |
| I-752 | B | D | | | 2154.9271 | 1079.2 | 2155.4 | |
| I-753 | A | C | | + | 2136.9365 | 2138.9 | 2137 | |
| I-754 | A | C | | | 2110.8879 | 2112.9 | 2111.3 | |
| I-755 | A | C | | | 2186.9192 | 1046.2 | 1044.3 | |
| I-756 | A | D | | | 2186.9192 | 2188.9 | 2186.5 | |
| I-757 | A | | | | 2157.8749 | 2159.8 | 2157.7 | |
| I-758 | B | | | | 2157.8749 | 2159.9 | 2158.6 | |
| I-759 | B | | | | 2171.8905 | 2173.9 | 2171.4 | |
| I-760 | B | | | | 2171.8905 | 2173.9 | 2171.3 | |
| I-761 | A | | | | 2060.9458 | 2063.9 | 2062.4 | |
| I-762 | A | C | | | 2060.9458 | 2063.8 | 2061.8 | |
| I-763 | A | | | | 2021.055 | 2023.1 | 2021.4 | |
| I-764 | A | C | | | 2021.055 | 2023.1 | 2021.4 | |
| I-765 | B | | | | 2070.9342 | 2075 | 2072.9 | |
| I-766 | B | | | | 2070.9342 | 2073.9 | 2072.8 | |
| I-767 | A | | | | 2043.0394 | 2045.1 | 2043.5 | |
| I-768 | A | | | | 2043.0394 | 2045.2 | 2043.3 | |
| I-769 | A | | | | 2057.055 | 2059.2 | 2057.6 | |
| I-770 | A | C | | | 2057.055 | 2059.2 | 2057.1 | |
| I-771 | A | | | | 2063.0114 | 2065.1 | 2063.4 | |
| I-772 | A | C | | + | 2063.0114 | 2065.1 | 2063.3 | |
| I-773 | B | | | | 2063.0114 | 2065.2 | 2063.4 | |
| I-774 | C | D | | | 2063.0114 | 2065.1 | 2063.1 | |
| I-775 | A | C | | | 2033.055 | 2035.1 | 2033.5 | |
| I-776 | A | C | | | 2033.055 | 2035.2 | 2033.2 | |
| I-777 | B | | | | 2136.9519 | 2138.8 | 2136.9 | |
| I-778 | A | | | | 2150.9675 | 2152.8 | 2150.6 | |
| I-779 | B | | | | 2150.9675 | 2152.8 | 2151.1 | |
| I-780 | A | | | | 2074.9209 | 2076.8 | 2074.8 | |
| I-781 | A | D | | | 2088.9365 | 2090.8 | 2088.8 | |
| I-782 | A | B | | | 2216.9951 | 2219 | 2216.7 | |
| I-783 | A | C | | | 2359.0693 | 2361.1 | 2359.7 | |
| I-784 | A | | | | 2501.1436 | 1252.3 | 1250.6 | |
| I-785 | A | B | | + | 2203.9999 | 2205.9 | 2204.2 | |
| I-786 | A | C | | + | 2017.8994 | 2019.7 | 2017.6 | |
| I-787 | A | B | | | 2088.9365 | 2090.8 | 2089.2 | |
| I-788 | A | C | | +++ | 1975.8525 | 1977.7 | 1975.8 | |
| I-789 | A | C | | + | 2188.9638 | 2191 | 2188.8 | |
| I-790 | A | C | | + | 2331.038 | 2333.1 | 2331.5 | |
| I-791 | A | | | | 2175.9686 | 2178 | 2175.7 | |
| I-792 | A | D | | | 1975.8525 | 1977.7 | 1975.6 | |
| I-793 | A | D | | | 2046.8896 | 2048.9 | 2046.8 | |
| I-794 | A | | | | 2046.8896 | 2077 | 2075.8 | |
| I-795 | A | D | | +++ | 2188.9638 | 2191.1 | 2189.3 | |
| I-796 | A | | | | 2188.9638 | 2191 | 2189 | |
| I-797 | A | | | | 2175.9686 | 2178.2 | 2176 | |
| I-798 | A | | | | 2175.9686 | 2178.1 | 2176.2 | |
| I-799 | A | C | | | 2102.9522 | 2105 | 2103.4 | |
| I-800 | A | C | | | 2102.9522 | 2105 | 2103.1 | |
| I-801 | A | D | | | 2116.9678 | 2119 | 2117.1 | |
| I-802 | A | C | | | 2103.9474 | 2106 | 2104.2 | |
| I-803 | A | A | | | 2080.9586 | 2082.9 | 2081 | |
| I-804 | A | A | | | 2259.0421 | 2261.1 | 2259.4 | |
| I-805 | A | B | | + | 2259.0421 | 2261.1 | 2259 | |
| I-806 | A | B | | + | 2372.1261 | 2374.2 | 2371.9 | |
| I-807 | A | C | | | 2330.0792 | 2332.1 | 2330.3 | |
| I-808 | B | | | | 2443.1632 | 2445.3 | 2442.9 | |
| I-809 | B | | | | 2443.1632 | 2445.3 | 2443.4 | |
| I-810 | B | | | | 2556.2473 | 1280 | 1278.2 | |
| I-811 | B | | | | 2556.2473 | 1280 | 1278 | |
| I-812 | A | B | | + | 2613.2688 | 1308.4 | 1306.7 | |
| I-813 | A | B | | + | 2684.3059 | 1344 | 1342.1 | |
| I-814 | A | C | | + | 2625.3052 | 1314.4 | 1312.7 | |
| I-815 | A | C | | + | 2696.3423 | 1350 | 1348.4 | |
| I-816 | A | A | | + | 2611.2644 | 1307.4 | 1305.9 | |
| I-817 | A | B | | + | 2682.3015 | 1342.9 | 1341.3 | |
| I-818 | A | D | | | 2155.9424 | 2157.8 | 2155.4 | A |
| I-819 | B | | | | 2074.9209 | 2076.8 | 2074.9 | |
| I-820 | A | C | | | 2150.9675 | 2153.2 | 2151.2 | |
| I-821 | A | | | | 2150.9675 | 2152.9 | 2151.1 | |
| I-822 | A | B | | | 2237.9995 | 2240.2 | 2238.2 | |
| I-823 | A | C | | | 2134.8862 | 2137.9 | 2135.9 | |
| I-824 | A | | | | 2162.9175 | 2166 | 2164.7 | |
| I-825 | A | C | | | 2162.9175 | 2165.6 | 2163.9 | |
| I-826 | A | C | | | 2249.9495 | 2252.8 | 2250.7 | |
| I-827 | A | C | | | 2176.9331 | 2180.1 | 2178.8 | |
| I-828 | B | | | | 2176.9331 | 1090.6 | 1088.7 | |
| I-829 | B | | | | 2263.9652 | 2267.1 | 2265.4 | |
| I-830 | B | | | | 2263.9652 | 1134.1 | 1132.5 | |
| I-831 | A | C | | | 2066.8988 | 2069.8 | 2068.2 | |
| I-832 | A | C | | | 2066.8988 | 1035.5 | 1034.1 | |
| I-833 | A | C | | | 2094.9301 | 2097.8 | 2096.2 | |
| I-834 | A | B | | | 2094.9301 | 1049.5 | 1047.1 | |
| I-835 | A | C | | | 2181.9621 | 2184.9 | 2183.1 | |
| I-836 | A | C | | | 2181.9621 | 1093.2 | 1090.8 | |
| I-837 | A | C | | | 2108.9458 | 2112 | 2109.4 | |
| I-838 | A | C | | | 2108.9458 | 2111.8 | 2109.3 | |
| I-839 | A | B | | | 2195.9778 | 2198.9 | 2197.2 | |
| I-840 | A | C | | | 2195.9778 | 2198.9 | | |
| I-841 | A | B | | | 2162.9522 | 2165.3 | 2163.4 | |
| I-842 | A | B | | | 2122.9209 | 2125.2 | 2123.2 | |
| I-843 | A | B | | | 2134.9209 | 2137.2 | 2135 | |
| I-844 | A | B | | | 2160.9365 | 2163.2 | 2160.9 | |
| I-845 | A | C | | | 2244.9365 | 1124.3 | 1122.7 | |
| I-846 | A | C | | | 2174.9522 | 2177.3 | 2175.9 | |
| I-847 | A | C | | | 2146.9209 | 2149.2 | 2147.1 | |
| I-848 | A | C | | | 2134.9209 | 2137.2 | 2135.3 | |
| I-849 | A | | | | 2100.9365 | 2102.9 | 2101.2 | |
| I-850 | A | B | | | 2126.9522 | 2129.1 | 2127.1 | |
| I-851 | A | | | | 2210.9522 | 2213.2 | 2210.8 | |
| I-852 | A | B | | | 2140.9678 | 2143.3 | 2141.1 | |
| I-853 | A | | | | 2140.9678 | 2142.9 | 2140.9 | |
| I-854 | A | | | | 2112.9365 | 2115.2 | 2112.9 | |
| I-855 | A | | | | 2100.9365 | 2102.9 | 2101.1 | |
| I-856 | A | | | | 2072.9052 | 2074.9 | 2073.2 | |
| I-857 | A | | | | 2098.9209 | 2101.2 | 2098.8 | |
| I-858 | A | | | | 2182.9209 | 1093.3 | 1091.3 | |
| I-859 | A | B | | | 2112.9365 | 2115.1 | 2112.9 | |
| I-860 | A | C | | | 2084.9052 | 2086.9 | 2084.9 | |
| I-861 | A | C | | | 2072.9052 | 1038.2 | 1036.8 | |
| I-862 | A | B | | | 2077.8647 | 2080.7 | 2078.9 | |
| I-863 | A | | | | 2077.8647 | 2080.8 | 2079.5 | |
| I-864 | A | C | | | 2174.9175 | 1089.7 | 1087 | |
| I-865 | A | C | | | 2164.8968 | 1084.6 | 1082.6 | |
| I-866 | B | | | | 2224.9331 | 2228.3 | 2226.2 | |
| I-867 | A | A | | + | 2015.8338 | 1010.2 | 1008 | |
| I-868 | A | C | | + | 2112.8865 | 2116.2 | 2113.3 | |
| I-869 | A | B | | + | 2102.8658 | 2105.9 | 2103.4 | |
| I-870 | A | C | | | 2162.9022 | 2166.4 | 2164 | |
| I-871 | A | A | | + | 2009.8773 | 2013.5 | 2010.8 | |
| I-872 | A | C | | + | 2106.9301 | 2110 | 2108.5 | |
| I-873 | A | B | | | 2096.9094 | 2099.9 | 2098.3 | |
| I-874 | A | C | | | 2156.9458 | 2159.8 | 2158.3 | |
| I-875 | A | | | | 2038.8821 | 2041 | 2039.2 | |
| I-876 | A | | | | 2109.9192 | 2112.1 | 2110.2 | |
| I-877 | A | | | | 2238.9982 | 2241.2 | 2239.3 | |
| I-878 | A | | | | 2032.9256 | 2035 | 2033.1 | |
| I-879 | A | | | | 2103.9628 | 1053.8 | 1052 | |
| I-880 | A | | | | 2233.0417 | 2235.2 | 2233.5 | |
| I-881 | A | | | | 2050.832 | 2053.8 | 2052.9 | |
| I-882 | A | | | | 2121.8692 | 2124.8 | 2123.3 | |
| I-883 | A | | | | 2250.9481 | 2254 | 2252.6 | |
| I-884 | A | | | | 2044.8756 | 1024.6 | 1023 | |
| I-885 | A | | | | 2115.9127 | 2118.8 | 2117.4 | |
| I-886 | A | | | | 2244.9917 | 2247.9 | 2246.1 | |
| I-887 | A | | | | 2100.913 | 2103 | 2101.1 | |
| I-888 | B | | | | 2301.0291 | 1152.4 | 1150.4 | |
| I-889 | B | | | | 2301.0291 | 1152.4 | 1150.6 | |
| I-890 | A | | | | 2112.863 | 2115.8 | 2113.9 | |
| I-891 | A | C | | | 2112.863 | 1058.7 | 1057 | |
| I-892 | A | D | | | 2183.9001 | 1094.2 | 1091.8 | |
| I-893 | B | | | | 2312.9791 | 1158.8 | 1156.7 | |
| I-894 | A | C | | | 2018.91 | 2021 | 2019.3 | |
| I-895 | A | | | | 2018.91 | 2021.1 | 2019.3 | |
| I-896 | A | C | | | 2089.9471 | 2092.1 | 2090.2 | |
| I-897 | A | | | | 2089.9471 | 2092.1 | 2090.2 | |
| I-898 | A | C | | | 2219.0261 | 2221.3 | 2219.3 | |
| I-899 | A | | | | 2219.0261 | 1111.5 | 1109.5 | |
| I-900 | A | D | | | 2094.9413 | 2097.1 | 2095.2 | |
| I-901 | A | D | | | 2165.9784 | 2168.2 | 2166.3 | |
| I-902 | B | | | | 2295.0574 | 1149.4 | 1147.6 | |
| I-903 | A | D | | ++ | 2178.9332 | 1091.4 | 1089.5 | |
| I-904 | A | C | | | 2177.9379 | 2180.2 | 2178.1 | |
| I-905 | A | | | | 2177.9379 | 1090.8 | 1089 | |
| I-906 | A | | | | 2176.9427 | 1090.4 | 1087.9 | |
| I-907 | A | | | | 2138.927 | 2141.2 | 2139.4 | |
| I-908 | A | B | | + | 2179.9787 | 1091.9 | 1089.8 | |
| I-909 | A | C | | | 2138.927 | 2141.2 | 2139.4 | |
| I-910 | A | | | | 2138.927 | 2141.2 | 2139.4 | |
| I-911 | A | | | | 2138.927 | 2141.2 | 2139 | |
| I-912 | A | | | | 2138.927 | 2141.4 | 2139.7 | |
| I-913 | A | B | | | 2086.8709 | 2090.1 | 2089.3 | |
| I-914 | A | C | | | 2086.8709 | 1045.8 | 1044.2 | |
| I-915 | A | C | | | 2215.9499 | 2219 | 2217.6 | |
| I-916 | A | C | | | 2215.9499 | 1110.3 | 1108.6 | |
| I-917 | A | C | | | 2114.9022 | 2118 | 2116.6 | |
| I-918 | A | C | | | 2114.9022 | 2118.2 | 2116.3 | |
| I-919 | A | C | | | 2148.8865 | 2151.2 | 2150.3 | |
| I-920 | A | C | | | 2148.8865 | 2152 | 2150.1 | |
| I-921 | B | C | | | 2092.8273 | 2095.6 | 2093.9 | |
| I-922 | A | C | | | 2092.8273 | 2095.6 | 2093.9 | |
| I-923 | A | B | | | 2003.8838 | 2005.7 | 2003.5 | |
| I-924 | A | C | | | 2003.8838 | 2006.2 | 2004.4 | |
| I-925 | A | B | | | 2203.9999 | 2205.9 | 2204.1 | |
| I-926 | A | C | | | 2102.9522 | 2105.3 | 2103.4 | |
| I-927 | A | B | | | 2136.9365 | 2138.9 | 2136.9 | |
| I-928 | E | | | | 2106.9624 | 1055.3 | 1053.6 | |
| I-929 | C | | | | 2087.9525 | 2090.1 | 2088.1 | |
| I-930 | C | | | | 2087.9525 | 1045.8 | 2088.1 | |
| I-931 | E | | | | 2112.9188 | 1057.7 | 2112.5 | |
| I-932 | E | | | | 2036.9052 | 2039.1 | 2037.1 | A |
| I-933 | E | | | | 2036.8147 | 2039 | 2037.2 | |
| I-934 | E | | | | 2040.846 | 2042.7 | 2041 | A |
| I-935 | B | D | | | 2088.9365 | 2091.3 | 2089.2 | |
| I-936 | B | D | | | 2063.0114 | 2065.2 | 2063.2 | |
| I-937 | B | D | | | 2063.0114 | 2065.3 | 2063.2 | |
| I-938 | B | | | | 2077.0271 | 2079.3 | 2077.8 | |
| I-939 | C | | | | 2077.0271 | 1040.4 | 2077.2 | |
| I-940 | A | C | | | 2124.9365 | 2127.4 | 2125.3 | |
| I-941 | A | | | | 2124.9365 | 2127.3 | 2125.2 | |
| I-942 | A | D | | | 2074.9614 | 2077.9 | 2075.2 | |
| I-943 | B | | | | 2088.9771 | 1046.8 | 1044.9 | |
| I-944 | A | D | | | 2088.9771 | 1046.8 | 2090.1 | |
| I-945 | A | C | | | 2136.8865 | 2140.4 | 2138.5 | |
| I-946 | A | | | | 2136.9519 | 2139.2 | 2137.2 | |
| I-947 | A | D | | | 2060.9052 | 2063.3 | 2061.9 | |
| I-948 | A | D | | | 2074.9209 | 2077.2 | 2076.4 | |
| I-949 | B | | | | 2144.9958 | 2147 | 2145.1 | |
| I-950 | B | | | | 2156.9458 | 2159.7 | 2157.6 | |
| I-951 | B | | | | 2156.9458 | 2159.8 | 2157.4 | |
| I-952 | C | | | | 2274.0748 | 2276.2 | 2274.3 | |
| I-953 | C | | | | 2274.0748 | 2276.2 | 2274 | |
| I-954 | C | | | | 2286.0247 | 2289 | 2288.2 | |
| I-955 | B | B | | | 2178.0748 | 1091 | 2178.7 | |
| I-956 | A | | | | 2190.0247 | 1097.3 | 2191.4 | |
| I-957 | A | C | | | 2074.9209 | 1039.3 | 1037.6 | |
| I-958 | E | | | | 2088.9365 | 1046.3 | 1044.4 | |
| I-959 | E | | | | 2088.9365 | 1046.3 | 1044.4 | |
| I-960 | E | | | | 2088.9365 | 1046.3 | 1044.5 | |
| I-961 | A | D | | | 2088.9365 | 2091.1 | 2089.1 | |
| I-962 | E | | | | 2088.9365 | 2191.2 | 2089.2 | |
| I-963 | B | | | | 2078.9522 | 1041.3 | 1039.4 | A |
| I-964 | E | | | | 2092.9678 | 2095.1 | 2093.2 | A |
| I-965 | C | | | | 2092.9678 | 2095.2 | 2093.8 | A |
| I-966 | E | | | | 2092.9678 | 2095.3 | 2093.2 | A |
| I-967 | B | | | | 2092.9678 | 2095.2 | 2092.9 | |
| I-968 | E | | | | 2091.9838 | 2094.2 | 2091.7 | |
| I-969 | A | A | | | 2046.8896 | | | |
| I-970 | A | B | | + | 2046.8896 | | | |
| I-971 | A | B | | | 2074.9209 | | | |
| I-972 | A | | | | 2088.9365 | | | |
| I-973 | A | | | | 2088.9365 | | | |
| I-974 | A | | | | 2088.9365 | | | |
| I-975 | A | | | | 2116.9678 | | | |
| I-976 | A | | | | 2072.9052 | | | |
| I-977 | A | | | | 2072.9052 | | | |
| I-978 | A | | | | 2100.9365 | | | |
| I-979 | A | | | | 2114.9522 | | | |
| I-980 | A | | | | 2142.9835 | | | |
| I-981 | A | | | | 2142.9835 | | | |
| I-982 | A | B | | + | 2156.9052 | 2159.2 | 2157.5 | |
| I-983 | A | B | | | 2156.9052 | 2159.3 | 2157.2 | |
| I-984 | A | B | | + | 2184.9365 | 1094.4 | 2185.9 | |
| I-985 | A | A | | | 2198.9522 | 1101.4 | 1099.6 | |
| I-986 | A | A | | + | 2198.9522 | 2201.3 | 2198.9 | |
| I-987 | A | B | | + | 2058.8896 | 2061.1 | 2059.3 | |
| I-988 | A | B | | + | 2086.9209 | 2089.2 | 2087.6 | |
| I-989 | A | C | | + | 2100.9365 | 2103.2 | 2101.2 | |
| I-990 | A | C | | | 2100.9365 | 2103.2 | 2101.4 | |
| I-991 | A | | | +++ | 2128.9678 | 2131.3 | 2129.2 | |
| I-992 | E | | | | 2032.8739 | 2035.2 | 2033.2 | |
| I-993 | D | | | | 2008.8739 | 2011.1 | 2009.5 | |
| I-994 | A | | | | 2058.8896 | 2061.3 | 2059 | |
| I-995 | A | | | | 2058.8896 | 2061.2 | 2059.3 | |
| I-996 | A | B | | | 2072.9052 | 2075.2 | 2073.3 | |
| I-997 | A | | | | 2116.9315 | 2119.4 | 2116.8 | |
| I-998 | A | C | | | 2116.9315 | 2119.4 | 2116.8 | |
| I-999 | A | C | | | 2072.9052 | 2075.4 | 2073.7 | |
| I-1000 | A | | | | 2142.8896 | 2145.3 | 2142.6 | |
| I-1001 | A | | | | 2142.8896 | 2145.3 | 2142.4 | |
| I-1002 | A | | | | 2156.9052 | 2159.2 | 2157.3 | |
| I-1003 | A | | | | 2156.9052 | 2159.3 | 2157.4 | |
| I-1004 | A | C | | | 2186.9158 | 2189.3 | 2187 | |
| I-1005 | A | | | | 2200.9315 | 2203.4 | 2201 | |
| I-1006 | A | D | | | 2200.9315 | 2203.4 | 2200.9 | |
| I-1007 | A | | | | 2200.9315 | 2203.4 | 2201.2 | |
| I-1008 | A | C | | | 2156.9052 | 2159.4 | 2156.9 | |
| I-1009 | A | C | | | 2156.9052 | 2159.3 | 2157.4 | |
| I-1010 | A | B | | | 2072.8552 | 2076.9 | 2075.3 | |
| I-1011 | A | | | | 2116.9315 | 2119.2 | 2116.6 | |
| I-1012 | A | B | | | 2152.9315 | 2155.3 | 2153 | |
| I-1013 | A | C | | | 2104.9315 | 2107.2 | 2105.5 | |
| I-1014 | A | C | | | 2086.9209 | 2089.2 | 2087.6 | |
| I-1015 | A | C | | | 2122.9209 | 2125.3 | 2123.2 | |
| I-1016 | A | D | | | 2074.9209 | 2077.2 | 2075.1 | |
| I-1017 | A | D | | | 2142.9835 | 2145.5 | 2143.2 | |
| I-1018 | A | C | | | 2178.9835 | 2181.4 | 2178.9 | |
| I-1019 | B | D | | | 2130.9835 | 2133.4 | 2131.9 | |
| I-1020 | A | C | | | 2130.9471 | 2133.3 | 2131.7 | |
| I-1021 | B | D | | | 2118.9471 | 2121.3 | 2119.3 | |
| I-1022 | B | | | | | | | A |
| I-1023 | A | D | | | 2060.9052 | 2061.9 | 2061.9 | |
| I-1024 | E | | | | 2043.8651 | 2047.4 | 2045.4 | |
| I-1025 | E | | | | 2208.0100 | 2210.6 | 2208.4 | |
| I-1026 | A | D | | | 2157.9580 | 2160.5 | 2158.5 | |
| I-1027 | A | D | | +++ | 2157.9580 | 2060.5 | 2158.0 | |
| I-1028 | A | D | | | 2219.9737 | 2222.7 | 2220.6 | |
| I-1029 | A | | | | 2219.9737 | 2222.6 | 2220.8 | |
| I-1030 | A | | | | 2200.0050 | 2202.7 | 2201.0 | |
| I-1031 | B | | | | 2143.9424 | 1074.0 | 1072.0 | |
| I-1032 | B | | | | 2205.9580 | 1105.0 | 1102.9 | |
| I-1033 | B | | | | 2185.9893 | 1095.0 | 1092.9 | |
| I-1034 | A | | | | 2143.9424 | 2146.4 | 2144.9 | |
| I-1035 | A | | | | 2205.9580 | 1105.1 | 1103.3 | |
| I-1036 | A | | | | 2185.9893 | 2188.6 | 2185.9 | |
| I-1037 | A | | | + | 2027.8338 | 2031.1 | 2029.4 | |
| I-1038 | A | | | + | 2041.8494 | 2044.9 | 2043.6 | |
| I-1039 | A | | | | 2041.8494 | 2044.9 | 2043.6 | |
| I-1040 | A | | | + | 2027.8338 | 2031.0 | 2029.4 | |
| I-1041 | A | | | | 2027.8338 | 2031.1 | 2029.3 | |
| I-1042 | A | | | + | 2041.8494 | 2045.2 | 2042.2 | |
| I-1043 | A | | | | 2041.8494 | 2045.1 | 2043.3 | |
| I-1044 | A | | | + | 2053.8494 | 2056.9 | 2054.4 | |
| I-1045 | A | | | | 2053.8494 | 2057.1 | 2054.9 | |
| I-1046 | A | | | | 2067.8651 | 2071.4 | 2069.4 | |
| I-1047 | A | | | + | 2067.8651 | 2071.2 | 2068.5 | |
| I-1048 | A | | | + | 2021.8773 | 2025.2 | 2023.2 | |
| I-1049 | A | | | | 2021.8773 | 2025.4 | 2024.6 | |
| I-1050 | A | | | + | 2035.8930 | 2039.0 | 2037.3 | |
| I-1051 | A | | | | 2035.8930 | 2039.1 | 2036.8 | |
| I-1052 | A | | | + | 2021.8773 | 2024.9 | 2022.7 | |
| I-1053 | A | | | | 2021.8773 | 2025.0 | 2023.7 | |
| I-1054 | A | | | + | 2035.8930 | 2039.1 | 2037.6 | |
| I-1055 | A | | | | 2035.8930 | 2039.6 | 2037.4 | |
| I-1056 | A | | | + | 2047.8930 | 2051.0 | 2049.1 | |
| I-1057 | A | | | | 2047.8930 | 2051.5 | 2049.1 | |
| I-1058 | A | | | ND | 2061.9086 | 2065.1 | 2063.0 | |
| I-1059 | A | | | | 2061.9086 | 2065.0 | 2063.2 | |
| I-1060 | A | | | | 1947.8617 | 1951.0 | 1948.2 | |
| I-1061 | A | | | | 1961.8773 | 1964.9 | 1963.5 | |
| I-1062 | A | | | | 2140.9427 | 2143.5 | 2141.2 | |
| I-1063 | A | | | | 2187.9286 | 2190.2 | 2188.8 | |
| I-1064 | A | | | +++ | 2190.9583 | 2193.6 | 2190.9 | |
| I-1065 | A | | | | 2155.9536 | 2158.5 | 2156.9 | |
| I-1066 | A | | | | 2201.9631 | 2204.8 | 2202.1 | |
| I-1067 | A | D | | | 2151.9474 | 2154.7 | 2152.7 | |
| I-1068 | A | | | | 2141.9379 | 1073.0 | 1070.8 | |
| I-1069 | A | | | | 2140.9427 | 2143.5 | 2141.6 | |
| I-1070 | A | | | | 2165.9631 | 2168.8 | 2167.7 | |
| I-1071 | A | | | +++ | 2165.9631 | 1082.2 | 1093.4 | |
| I-1072 | A | | | | 2165.9631 | 2168.7 | 2167.0 | |
| I-1073 | A | | | | 2152.9427 | 2155.6 | 2154.1 | |
| I-1074 | A | | | | 2157.9580 | 2160.3 | 2158.4 | |
| I-1075 | A | | | +++ | 2141.9379 | 2144.3 | 2142.2 | |
| I-1076 | A | D | | | 2190.9583 | 2193.8 | 2191.4 | |
| I-1077 | A | | | | 2190.9583 | 2193.6 | 2191.6 | |
| I-1078 | A | | | +++ | 2117.9631 | 2120.7 | 2118.1 | |
| I-1079 | A | | | +++ | 2187.0209 | 2190.0 | 2187.8 | |
| I-1080 | A | | | | 2187.0209 | 1095.6 | 1093.4 | |
| I-1081 | A | | | +++ | 2173.0053 | 2175.6 | 2173.5 | |
| I-1082 | A | | | | 2173.0053 | 2175.8 | 2173.8 | |
| I-1083 | A | | | | 2176.9097 | 1090.6 | 1088.6 | |
| I-1084 | A | | | | 2176.9097 | 1090.6 | 1088.9 | |
| I-1085 | A | | | | 2223.9144 | 2226.5 | 2224.2 | |
| I-1086 | A | | | | 2025.8293 | 2029.1 | | |
| I-1087 | A | | | | 2025.8293 | 2029.0 | 2027.8 | |
| I-1088 | A | | | | 2096.8665 | 2100.2 | | |
| I-1089 | A | | | | 2011.8137 | 2015.0 | 2012.6 | |
| I-1090 | A | | | | 2073.8293 | 2077.1 | 2074.9 | |
| I-1091 | A | | | | 2144.8665 | 2148.2 | | |
| I-1092 | A | | | | 2019.8729 | 2023.1 | 2020.6 | |
| I-1093 | A | | | | 2090.9100 | 2094.7 | 2092.3 | |
| I-1094 | A | | | | 2005.8573 | 2009.1 | | |
| I-1095 | A | | | | 2076.8944 | 2080.1 | 2078.2 | |
| I-1096 | A | | | | 2067.8729 | 2071.2 | 2069.4 | |
| I-1097 | A | | | | 2138.9100 | 2142.1 | 2140.5 | |
| I-1098 | A | | | | 1975.9430 | 1978.2 | 1976.6 | |
| I-1099 | A | | | | 2032.9645 | 2035.3 | 2033.8 | |
| I-1100 | A | | | | 1961.9274 | 1964.2 | 1962.8 | |
| I-1101 | A | | | | 2094.9801 | 2097.3 | 2095.4 | |
| I-1102 | A | | | + | 2023.9430 | 2026.3 | 2024.9 | |
| I-1103 | A | | | + | 1969.9866 | 1972.3 | 1970.1 | |
| I-1104 | A | | | | 2027.0081 | 2029.3 | 2027.7 | |
| I-1105 | A | | | | 1955.9709 | 1958.2 | 1956.5 | |
| I-1106 | A | | | + | 2089.0237 | 2091.4 | 2089.3 | |
| I-1107 | A | | | + | 2017.9866 | 2020.3 | 2018.6 | |
| I-1108 | A | | | + | 2111.8338 | 2115.3 | 2113.8 | |
| I-1109 | A | | | | 2182.8709 | 2186.3 | 2185.1 | |
| I-1110 | A | | | | 2198.8658 | 2202.1 | 2200.3 | |
| I-1111 | A | | | + | 2097.8181 | 2101.6 | 2099.3 | |
| I-1112 | A | | | | 2168.8552 | 2172.1 | 2170.3 | |
| I-1113 | A | | | | 2184.8501 | 2188.3 | 2185.9 | |
| I-1114 | A | | | | 2173.8494 | 2178.2 | 2176.5 | |
| I-1115 | A | | | | 2244.8865 | 2247.5 | | |
| I-1116 | A | | | | 2260.8814 | 1032.9 | 1030.7 | |
| I-1117 | A | | | + | 2137.8494 | 1071.3 | 2139.3 | |
| I-1118 | A | | | + | 2208.8865 | 1106.8 | 2210.1 | |
| I-1119 | A | | | | 2224.8814 | 2228.1 | 2226.4 | |
| I-1120 | A | | | | 2126.9634 | 2129.4 | 2127.3 | |
| I-1121 | A | | | | 2126.9634 | 2129.4 | 2127.4 | |
| I-1122 | B | | | | 2098.9321 | 2103.4 | 2101.6 | |
| I-1123 | B | | | | 2098.9321 | 2103.4 | 2101.8 | |
| I-1124 | A | | | | 2110.9685 | 2113.4 | | |
| I-1125 | A | | | | 2082.9372 | 2085.3 | 2083.4 | |
| I-1126 | A | | | | 2111.9274 | 2114.3 | 2111.9 | |
| I-1127 | A | | | | 2111.9274 | 2114.3 | 2112.1 | |
| I-1128 | A | D | | | 2163.9686 | 2166.4 | 2164.3 | |
| I-1129 | A | D | | | 2163.9686 | 2166.4 | 2164.6 | |
| I-1130 | E | D | | | 2176.0050 | 2155.9 | 2154.0 | |
| I-1131 | E | D | | | 2176.0050 | 2114.0 | 2112.7 | |
| I-1132 | A | D | | | 2190.0206 | 2192.6 | 2190.7 | |
| I-1133 | A | D | +++ | | 2190.0206 | 2192.5 | 2190.6 | |
| I-1134 | A | D | +++ | | 2177.9842 | 2180.5 | 2178.6 | |
| I-1135 | A | D | | | 2177.9842 | 2180.5 | 2179.0 | |
| I-1136 | A | D | | | 2224.0050 | 2226.5 | 2224.7 | |
| I-1137 | B | D | | | 2224.0050 | 2226.6 | 2224.9 | |
| I-1138 | A | D | | | 2245.0264 | 2247.5 | 2245.3 | |
| I-1139 | A | D | | | 2245.0264 | 2247.6 | 2245.8 | |
| I-1140 | A | D | | | 2235.0057 | 2237.5 | 2235.2 | |
| I-1141 | A | D | | | 2235.0057 | 1119.6 | 1117.7 | |
| I-1142 | A | D | | | 2219.0108 | 2221.5 | 2219.9 | |
| I-1143 | A | D | | | 2219.0108 | 2221.5 | 2219.4 | |
| I-1144 | A | D | | +++ | 2167.9999 | 2170.5 | 2168.6 | A |
| I-1145 | A | D | ++ | + | 2180.0363 | 2180.6 | 2178.6 | A |
| I-1146 | A | D | | + | 2194.0519 | 2196.4 | 2194.1 | A |
| I-1147 | A | D | | +++ | 2182.0155 | 2184.5 | 2182.4 | A |
| I-1148 | A | D | | | 2228.0363 | 2230.4 | 2228.5 | A |
| I-1149 | B | D | | +++ | 2249.0577 | 2251.4 | 2249.6 | A |
| I-1150 | A | D | | +++ | 2239.0370 | 2241.5 | 2239.0 | A |
| I-1151 | A | D | | +++ | 2223.0421 | 2225.3 | 2223.4 | A |
| I-1152 | | | | | 2058.9624 | 2061.4 | 2059.2 | |
| I-1153 | E | | | | 2086.9937 | 2089.2 | 2087.3 | |
| I-1154 | E | | | | 2162.9344 | 2165.5 | 2163.2 | |
| I-1155 | A | | | + | 2586.2076 | 1295.1 | 1293.5 | |
| I-1156 | A | | | + | 2671.2716 | 1337.6 | 1336.2 | |
| I-1157 | A | | | + | 2671.2716 | 1337.7 | 1335.9 | |
| I-1158 | A | | | + | 2615.2593 | 1309.7 | 1307.7 | |
| I-1159 | A | | | | 2598.1963 | 1301.0 | 1299.2 | |
| I-1160 | A | | | | 2669.2335 | 1336.6 | 1334.5 | |
| I-1161 | B | | | | 2123.0114 | 2125.4 | 2123.0 | |
| I-1162 | A | | | +++ | 2002.8998 | 2005.3 | 2003.7 | |
| I-1163 | A | | | +++ | 2098.8998 | 2101.4 | | |
| I-1164 | A | | | +++ | 2169.9369 | 2172.5 | 2170.7 | |
| I-1165 | A | | | +++ | 1988.8841 | 1991.3 | 1989.3 | |
| I-1166 | A | | | +++ | 1988.8841 | 1991.3 | 1989.6 | |
| I-1167 | A | | | +++ | 2059.9212 | 2062.3 | 2060.0 | |
| I-1168 | A | | | +++ | 2059.9212 | 2062.4 | 2060.4 | |
| I-1169 | A | | | +++ | 2075.9161 | 2078.3 | 2076.3 | |
| I-1170 | A | | | +++ | 2075.9161 | 2078.4 | 2076.5 | |
| I-1171 | A | | | +++ | 2008.8933 | 2012.0 | | |
| I-1172 | A | | | +++ | 2008.8933 | 2012.0 | 2009.9 | |
| I-1173 | A | | | +++ | 2079.9304 | 2083.1 | 2081.2 | |
| I-1174 | A | | | +++ | 2079.9304 | 2083.1 | 2081.4 | |
| I-1175 | A | | | +++ | 2104.8933 | 1054.7 | | |
| I-1176 | A | | | | 2104.8933 | 2108.1 | 2106.7 | |
| I-1177 | A | | | +++ | 2175.9304 | 1090.3 | 2176.1 | |
| I-1178 | A | | | +++ | 1994.8777 | 1998.0 | 1996.3 | |
| I-1179 | A | | | | 1994.8777 | 1998.0 | 1996.3 | |
| I-1180 | A | | | +++ | 2065.9148 | 2069.1 | 2066.7 | |
| I-1181 | A | | | +++ | 2350.0942 | 2352.7 | 2351.1 | |
| I-1182 | A | | | +++ | 2526.1990 | 1265.1 | 1263.5 | |
| I-1183 | A | | | + | 2878.4087 | 1441.3 | 1439.5 | |
| I-1184 | A | | | +++ | 2251.0258 | 2253.6 | 2251.6 | |
| I-1185 | A | | | +++ | 2427.1306 | 2429.8 | 2427.8 | |
| I-1186 | A | | | +++ | 2779.3403 | 1391.8 | 1389.8 | |
| I-1187 | A | | | +++ | 3131.5500 | 1568.0 | 1566.2 | |
| I-1188 | B | | | | 2074.9380 | 1039.3 | 1037.7 | |
| I-1189 | B | | | | 2074.9380 | 1039.2 | 1037.2 | |
| I-1190 | B | | | | 2074.9380 | 2077.1 | 2075.7 | |
| I-1191 | C | | | | 2161.9893 | 2164.4 | | |
| I-1192 | C | | | | 2161.9893 | 2164.4 | 2162.1 | |
| I-1193 | C | | | | 2146.9896 | 2149.6 | 2147.7 | |
| I-1194 | B | | | | 2146.9896 | 2149.1 | 2147.5 | |
| I-1195 | C | | | | 2119.9787 | 2122.4 | 2120.6 | |
| I-1196 | C | | | | 2119.9787 | 2122.3 | 2120.4 | |
| I-1197 | B | | | | 2133.9944 | 2136.4 | | |
| I-1198 | E | | | | 2133.9944 | 2136.4 | 2134.6 | |
| I-1199 | B | | | | 2161.9893 | 2164.4 | 2162.2 | |
| I-1200 | C | | | | 2161.9893 | 2164.5 | 2162.3 | |
| I-1201 | C | | | | 2146.9896 | 2149.4 | 2147.3 | |
| I-1202 | C | | | | 2146.9896 | 2149.4 | 2147.3 | |
| I-1203 | C | | | | 2119.9787 | 2122.3 | 2120.4 | |
| I-1204 | E | | | | 2119.9787 | 2122.4 | 2120.3 | |
| I-1205 | E | | | | 2133.9944 | 2136.4 | 2134.6 | |
| I-1206 | E | | | | 2133.9944 | 2136.4 | 2134.0 | |
| I-1207 | E | | | | 2176.0050 | 2178.4 | 2176.3 | |
| I-1208 | E | | | | 2176.0050 | 2178.4 | 2176.6 | |
| I-1209 | E | | | | 2219.9948 | 2222.4 | 2220.6 | |
| I-1210 | D | | | | 2219.9948 | 2222.4 | 2220.3 | |
| I-1211 | A | | | | 2219.9948 | 2222.5 | 2221.1 | |
| I-1212 | A | | | | 2219.9948 | 2223.3 | 2221.3 | |
| I-1213 | B | D | | | 2064.9729 | 2067.3 | 2065.6 | A |
| I-1214 | B | D | | | 2107.0199 | 2109.3 | 2107.3 | A |
| I-1215 | A | D | | | 2064.9729 | 2067.4 | 2064.8 | A |
| I-1216 | B | D | | | 2078.9886 | 2181.4 | 2179.3 | A |
| I-1217 | C | | | +++ | 2166.0206 | 2168.5 | 2166.2 | A |
| I-1218 | B | | | | 2151.0209 | 2153.6 | 2151.5 | A |
| I-1219 | E | | | | 2124.0100 | 2126.6 | 2124.7 | A |
| I-1220 | C | | | | 2138.0257 | 2140.4 | 2137.9 | A |
| I-1221 | D | | | +++ | 2166.0206 | 2168.5 | 2166.9 | A |
| I-1222 | C | | | | 2151.0209 | 2153.4 | 2151.3 | A |
| I-1223 | E | | | | 2124.0100 | 2126.4 | 2124.4 | A |
| I-1224 | E | | | | 2138.0257 | 2140.4 | 2138.5 | A |
| I-1225 | E | | | | 2180.0363 | 2182.4 | 2180.2 | A |
| I-1226 | D | | | | 2224.0261 | 2226.5 | 2224.4 | A |
| I-1227 | B | | | | 2224.0261 | 2226.5 | | A |
| I-1228 | E | | | | 2238.0417 | 2240.8 | 2238.9 | A |
| I-1229 | A | | | | 2157.9985 | 2161.2 | 2158.8 | A |
| I-1230 | A | | | + | 2086.9614 | 2090.4 | 2088.6 | A |
| I-1231 | A | | | + | 2163.9549 | 2167.1 | | A |
| I-1232 | A | | | + | 2092.9178 | 2096.0 | 2094.3 | A |
| I-1233 | A | | | | 2143.9829 | 2147.1 | 2145.4 | A |
| I-1234 | A | | | | 2072.9458 | 1038.8 | 1037.1 | A |
| I-1235 | A | | | | 2101.9359 | 1053.3 | 1051.3 | A |
| I-1236 | A | | | | 2030.8988 | 2033.9 | 2032.4 | A |
| I-1237 | A | | | | 2177.9672 | 2181.2 | 2180.2 | A |
| I-1238 | A | | | | 2106.9301 | 2110.4 | 2108.2 | A |
| I-1239 | A | | | | 2129.9672 | 2133.0 | 2131.5 | A |
| I-1240 | B | | | | 2058.9301 | 1031.7 | 2060.5 | A |
| I-1241 | A | | | | 2130.9413 | 1067.8 | 1066.0 | |
| I-1242 | A | | | | 1997.9274 | 2000.1 | 1998.1 | |
| I-1243 | A | | | | 1983.9117 | 1986.1 | 1984.5 | |
| I-1244 | A | | | | 2054.9488 | 2057.2 | 2055.1 | |
| I-1245 | A | | | | 2015.9179 | 2018.2 | 2016.2 | |
| I-1246 | A | | | | 2015.9179 | 2018.2 | 2016.8 | |
| I-1247 | A | | | | 2086.9551 | 2089.2 | 2087.1 | |
| I-1248 | A | | | | 2031.8884 | 2034.6 | 2032.3 | |
| I-1249 | A | | | | 2102.9255 | 2105.8 | 2104.4 | |
| I-1250 | A | | | | 2011.9430 | 2085.3 | 2083.3 | |
| I-1251 | A | | | | 2082.9801 | 2014.2 | 2012.6 | |
| I-1252 | A | | | | 2295.0098 | 2298.3 | 2296.0 | |
| I-1253 | A | | | | 2295.0098 | 2298.3 | 2295.8 | |
| I-1254 | A | | | | 2337.0568 | 1170.9 | 1169.2 | |
| I-1255 | A | | | | 2337.0568 | 2340.4 | 2338.6 | |
| I-1256 | A | | | | 2267.0149 | 1135.8 | 2268.1 | |
| I-1257 | A | | | | 2267.0149 | 1135.9 | 1134.3 | |
| I-1258 | A | | | | 2321.0255 | 2324.3 | 2322.0 | |
| I-1259 | A | | | | 2321.0255 | 2324.4 | 2321.8 | |
| I-1260 | A | | | | 2363.0724 | 2366.6 | 2364.3 | |
| I-1261 | A | | | | 2363.0724 | 1183.8 | 1182.1 | |
| I-1262 | A | | | | 2293.0305 | 1148.8 | 1147.0 | |
| I-1263 | A | | | | 2300.9662 | 2304.3 | 2302.7 | |
| I-1264 | A | | | | 2343.0132 | 1173.9 | | |
| I-1265 | A | | | | 2272.9713 | 1138.8 | 1136.2 | |
| I-1266 | A | | | | 2326.9819 | 2330.3 | 2328.5 | |
| I-1267 | A | | | | 2369.0288 | 2372.5 | 2370.3 | |
| I-1268 | A | | | + | 2298.9870 | 1152.0 | 2299.9 | |
| I-1269 | B | | | | 2233.9062 | 1119.0 | 2234.1 | |
| I-1270 | C | | | | 2207.8905 | 1106.0 | 1103.7 | |
| I-1271 | A | | | | 2144.8545 | | | |
| I-1272 | A | | | | 2143.8592 | | | |
| I-1273 | A | | | + | 2158.8701 | 2161.4 | 2159.4 | |
| I-1274 | A | | | + | 2081.8436 | 2084.2 | 2082.0 | |
| I-1275 | A | | | | 2095.8592 | 2098.3 | | |
| I-1276 | A | | | | 2137.8698 | 2140.3 | | |
| I-1277 | A | | | | 2123.8541 | 1063.9 | 1062.0 | |
| I-1278 | A | | | + | 2093.8436 | 2096.3 | 2094.4 | |
| I-1279 | A | | +++ | | 2095.8592 | 2098.4 | 2096.9 | |
| I-1280 | A | | ++ | | 2109.8749 | 2112.4 | 2110.3 | |
| I-1281 | A | | ++ | | 2107.8592 | 2110.4 | | |
| I-1282 | A | | | | 2095.8228 | 2098.3 | 2096.4 | |
| I-1283 | A | | | | 2109.8385 | 1056.8 | 2110.4 | |
| I-1284 | A | | +++ | | 2294.0792 | 2296.6 | 2294.2 | A |
| I-1285 | A | | | | 2338.0690 | 2340.7 | 2339.0 | A |
| I-1286 | B | | | | 2320.0948 | 2322.6 | 2320.3 | A |
| I-1287 | B | | | | 2310.0741 | 2312.8 | 2311.2 | A |
| I-1288 | A | | | | 2354.0639 | 2356.6 | 2354.9 | A |
| I-1289 | B | | | | 2336.0897 | 2338.6 | | A |
| I-1290 | B | D | | + | 2336.1261 | 2338.6 | 2336.4 | A |
| I-1291 | A | D | | + | 2380.1160 | 2282.7 | 2281.1 | A |
| I-1292 | B | | | + | 2362.1418 | 2364.6 | 2362.3 | A |
| I-1293 | B | | | | 2310.0741 | 2312.7 | 2310.7 | A |
| I-1294 | A | | | | 2354.0639 | 2356.5 | | A |
| I-1295 | B | | | | 2336.0897 | 2338.6 | | A |
| I-1296 | B | | | | 2114.9927 | 2118.0 | | A |
| I-1297 | E | | | | 2251.1098 | 2253.6 | 2251.9 | A |
| I-1298 | E | | | | 2265.1254 | 2267.6 | 2265.4 | A |
| I-1299 | B | | +++ | | 2180.0726 | 1092.0 | 1090.4 | A |
| I-1300 | B | | +++ | | 2194.0883 | 2096.5 | 2094.7 | A |
| I-1301 | B | | | | 2090.8980 | 1046.4 | 1044.5 | |
| I-1302 | C | | | | 2090.8980 | 2091.3 | 2089.3 | |
| I-1303 | E | | | | 2106.8752 | 2106.1 | 2104.1 | |
| I-1304 | A | | | | 2063.0114 | 2065.3 | 2063.4 | |
| I-1305 | B | | | | 2082.9801 | 2085.3 | 2083.4 | |
| I-1306 | A | | | | 2036.9594 | 2039.2 | 2037.3 | |
| I-1307 | A | | | | 2074.9614 | 2078.1 | 2076.4 | |
| I-1308 | A | | | | 2094.9301 | 2098.1 | 2096.2 | |
| I-1309 | A | | | | 2048.9094 | 2052.1 | 2050.6 | |
| I-1310 | A | | | | 2048.9958 | 2051.3 | 2049.5 | |
| I-1311 | B | | | | 2068.9645 | 2071.3 | | |
| I-1312 | B | | | | 2068.9645 | 2071.3 | 2069.3 | |
| I-1313 | A | | | | 2022.9437 | 2025.3 | 2023.1 | |
| I-1314 | A | | | | 2060.9458 | 2064.3 | | |
| I-1315 | A | | | | 2080.9145 | 2084.0 | 2081.7 | |
| I-1316 | B | | | | 2034.8937 | 2037.9 | 2036.0 | |
| I-1317 | A | | | | 2060.9958 | 2063.3 | 2061.3 | |
| I-1318 | A | | | | 2075.0114 | 2077.4 | 2075.8 | |
| I-1319 | B | | | | 2089.0271 | 2091.3 | 2089.3 | |
| I-1320 | A | | | | 2072.9458 | 2076.1 | | |
| I-1321 | A | | | | 2086.9614 | 2090.0 | 8087.8 | |
| I-1322 | A | | | | 2100.9771 | 2104.2 | | |
| I-1323 | A | | | | 2046.9801 | 2049.3 | 2047.0 | |
| I-1324 | A | | | | 2060.9958 | 2063.2 | 2061.3 | |
| I-1325 | B | | | | 2075.0114 | 2077.3 | 2075.5 | |
| I-1326 | A | | | | 2058.9301 | 2062.0 | 2060.5 | |
| I-1327 | A | | | | 2072.9458 | 2076.1 | 2074.6 | |
| I-1328 | A | | | | 2086.9614 | 2090.1 | | |
| I-1329 | A | | | | 2075.9737 | 2078.3 | 2076.3 | A |
| I-1330 | C | | | | 2089.9893 | 2092.3 | 2090.0 | A |
| I-1331 | E | | | | 2187.0032 | 2189.4 | 2187.6 | A |
| I-1332 | A | | | | 2101.9893 | 2104.4 | 2101.9 | A |
| I-1333 | C | | | | 2116.0050 | 2118.4 | 2116.2 | A |
| I-1334 | E | | | | 2213.0189 | 2215.3 | 2213.5 | A |
| I-1335 | A | | | | 2072.9458 | 1038.6 | 1036.8 | A |
| I-1336 | B | | | | 2072.9458 | 2076.3 | 2072.8 | A |
| I-1337 | A | | | | 1996.9145 | 1000.6 | 998.8 | A |
| I-1338 | C | | | | 2010.9301 | 1007.6 | 1005.7 | A |
| I-1339 | E | | | | 1995.9304 | 1998.8 | 1995.9 | A |
| I-1340 | C | | | | 2009.9461 | 2012.8 | 2010.8 | A |
| I-1341 | C | | | | 1968.9195 | 1971.7 | 1970.3 | A |
| I-1342 | B | | | | 2114.9927 | 1059.7 | | A |
| I-1343 | B | | | | 2100.9771 | 2104.0 | 2102.0 | A |
| I-1344 | B | | | | 2114.9927 | 1059.8 | | A |
| I-1345 | A | | | + | 2289.1068 | 2291.6 | 2289.9 | |
| I-1346 | A | | | + | 2303.1224 | 2305.6 | 2303.5 | |
| I-1347 | A | | | +++ | 2317.1381 | 2319.6 | 2318.1 | |
| I-1348 | A | | | | 2301.0568 | 2304.4 | 2302.3 | |
| I-1349 | A | | | + | 2315.0724 | 2318.3 | | |
| I-1350 | A | | | + | 2329.0881 | 1166.9 | 1165.2 | |
| I-1351 | A | | | | 2296.9891 | 2300.4 | 2298.5 | |
| I-1352 | A | | | | 2282.9734 | 2286.4 | | |
| I-1353 | A | | | | 2254.9785 | 2258.3 | 2256.6 | |
| I-1354 | A | | | | 2240.9629 | 2244.3 | 2242.8 | |
| I-1355 | A | | | | 2296.0414 | 2299.4 | | |
| I-1356 | A | | | | 2282.0258 | 2285.5 | 2282.8 | |
| I-1357 | A | | | | 2339.0360 | 2342.4 | 2340.0 | |
| I-1358 | A | | | | 2325.0204 | 2328.5 | 2326.8 | |
| I-1359 | A | | | | 2297.0255 | 1150.7 | 1148.8 | |
| I-1360 | A | | | | 2283.0098 | 2286.3 | | |
| I-1361 | A | | | | 2338.0884 | 2341.5 | | |
| I-1362 | A | | | | 2324.0727 | 2327.5 | | |
| I-1363 | B | | +++ | | 2102.9886 | 2105.4 | 2103.5 | |
| I-1364 | B | | +++ | | 2074.9573 | 2077.1 | | |
| I-1365 | A | | +++ | | 2157.8749 | 2060.2 | 2158.1 | |
| I-1366 | C | | +++ | | 2171.8905 | 1087.9 | 1086.2 | |
| I-1367 | A | | | + | 1995.8617 | 1999.0 | 1997.4 | |
| I-1368 | A | | | | 1995.8617 | 1999.1 | | |
| I-1369 | A | | | + | 2009.8773 | 2013.0 | | |
| I-1370 | A | | | + | 2009.8773 | 2012.9 | 2011.5 | |
| I-1371 | A | | | +++ | 2009.8773 | 2013.0 | 2011.4 | |
| I-1372 | A | | | + | 2009.8773 | 2013.0 | 2011.1 | |
| I-1373 | A | | | +++ | 2023.8930 | 2027.0 | | |
| I-1374 | A | | | | 2023.8930 | 1014.2 | 2025.2 | |
| I-1375 | A | | | | 2023.8930 | 2027.1 | 2025.5 | |
| I-1376 | A | | | + | 2024.8519 | 2028.0 | 2026.2 | |
| I-1377 | A | | | | 2024.8519 | 2027.9 | 2026.0 | |
| I-1378 | A | | | | 2038.8675 | 2042.0 | 2040.5 | |
| I-1379 | A | | | | 2050.8675 | 2054.0 | 2052.7 | |
| I-1380 | A | | | | 2064.8832 | 2068.7 | 2066.8 | |
| I-1381 | A | | | | 2064.8832 | 2068.0 | 2066.6 | |
| I-1382 | A | | | | 2064.8832 | 2068.1 | 2066.2 | |
| I-1383 | A | | | | 2064.8832 | 2068.0 | | |
| I-1384 | A | | | | 2064.8832 | 2068.0 | 2066.3 | |
| I-1385 | A | | | | 2064.8832 | 2068.0 | 2066.4 | |
| I-1386 | B | | | | 2157.8749 | 2160.4 | 2158.6 | |
| I-1387 | B | | | | 2158.8701 | 2161.4 | 2159.7 | |
| I-1388 | B | | | | 2095.8592 | 2098.2 | 2096.5 | |
| I-1389 | B | | | | 2107.8592 | 2110.4 | | |
| I-1390 | B | | | | 2171.8905 | 2174.3 | 2172.6 | |
| I-1391 | B | | | | 2137.8698 | 2140.4 | 2138.2 | |
| I-1392 | A | | | | 2316.0635 | 2318.6 | | |
| I-1393 | A | | | | 2308.0414 | 2311.3 | 2309.8 | |
| I-1394 | A | | | | 2387.1006 | 2389.7 | 2388.2 | |
| I-1395 | A | | | | 2379.0786 | 1192.0 | 1190.3 | |
| I-1396 | A | | | | 2011.9212 | 2014.2 | 2011.9 | |
| I-1397 | C | | | | 2067.9474 | 2070.5 | 2068.2 | |
| I-1398 | A | | | | 2207.9160 | 2210.4 | 2208.8 | |
| I-1399 | A | | | | 2137.9505 | 2140.4 | | |
| I-1400 | A | | | | 2088.9365 | 2091.4 | 2089.3 | |
| I-1401 | A | | | | 2088.9365 | 2091.3 | 2089.3 | |
| I-1402 | B | | | | 2492.1684 | 1248.1 | 1246.2 | |
| I-1403 | A | | | | 2668.2733 | 1336.1 | 1334.8 | |
| I-1404 | A | | | | 3020.4830 | 1512.4 | 1510.7 | |
| I-1405 | B | | | | 2484.1463 | 2487.6 | 2485.6 | |
| I-1406 | A | | | | 2660.2512 | 1332.5 | 1330.8 | |
| I-1407 | A | | | | 3012.4609 | 1508.8 | 1506.9 | |
| I-1408 | A | | | | 2739.3104 | 1371.7 | 1370.2 | |
| I-1409 | A | | | | 3091.5201 | 1547.9 | 1545.8 | |
| I-1410 | A | | | | 4016.0706 | 2010.5 | 2008.5 | |
| I-1411 | A | | | | 2555.1834 | 1280.0 | 1278.1 | |
| I-1412 | A | | | | 2731.2883 | 1368.1 | 1366.7 | |
| I-1413 | A | | | | 3083.4980 | 1544.3 | 1542.8 | |
| I-1414 | A | | | | 2066.8988 | 2070.0 | 2068.5 | |
| I-1415 | A | | | | 2052.8832 | 2056.0 | 2053.3 | |
| I-1416 | A | | | | 2038.8675 | 2041.9 | 2039.4 | |
| I-1417 | A | | | | 2038.8675 | 2041.9 | | |
| I-1418 | A | | | | 2038.8675 | 2042.0 | 2040.3 | |
| I-1419 | A | | | | 2050.8675 | 2054.0 | 2052.4 | |
| I-1420 | A | | | | 2036.8519 | 2039.9 | 2038.1 | |
| I-1421 | A | | | | 2050.8675 | 2054.0 | 2052.4 | |
| I-1422 | A | | | | 2036.8519 | 2054.0 | 2052.4 | |
| I-1423 | A | | | | 2050.8675 | 2054.0 | | |
| I-1424 | A | | | | 2036.8519 | 2040.0 | 2038.5 | |
| I-1425 | B | | | | 2172.8858 | 2175.6 | 2174.8 | |
| I-1426 | B | | | | 2185.9062 | 1094.8 | 1093.7 | |
| I-1427 | B | | | | 2109.8749 | 2112.4 | 2110.4 | |
| I-1428 | C | | | | 2123.8905 | 1063.8 | 2124.4 | |
| I-1429 | A | | | | 2121.8749 | 2123.3 | | |
| I-1430 | B | | | | 2123.8541 | 2126.7 | 2124.5 | |
| I-1431 | A | | | | 2069.9267 | 2072.3 | 2070.4 | |
| I-1432 | A | | | | 2055.9111 | 2058.3 | 2056.1 | |
| I-1433 | A | | | | 2083.9424 | 2086.3 | 2083.9 | |
| I-1434 | A | | | | 2111.9737 | 2114.3 | 2112.7 | |
| I-1435 | A | | | | 2083.9424 | 2086.3 | 2084.7 | |
| I-1436 | A | | | | 2111.9737 | 2114.4 | 2112.5 | |
| I-1437 | B | | | | 2102.9059 | 2105.3 | 2103.2 | |
| I-1438 | A | | | | 2132.9165 | 2135.4 | | |
| I-1439 | A | | | | 2132.9165 | 2135.4 | 2133.2 | |
| I-1440 | A | | | | 2139.9587 | 2142.4 | 2140.6 | |
| I-1441 | A | | | | 2139.9587 | 2142.4 | 2140.0 | |
| I-1442 | A | | | | 2139.9587 | 2142.3 | 2140.8 | |
| I-1443 | B | | | | 2111.9274 | 1058.3 | 2112.4 | |
| I-1444 | B | | | | 2151.9587 | 1263.3 | | |
| I-1445 | B | | | | 2151.9587 | 2154.2 | | |
| I-1446 | B | | | | 2151.9587 | 2154.4 | 2152.3 | |
| I-1447 | B | | | | 2151.9587 | 1078.8 | | |
| I-1448 | B | | | | 2137.9430 | 2140.2 | | |
| I-1449 | B | | | | 2144.9529 | 2147.4 | 2145.3 | |
| I-1450 | B | | | | 2116.9216 | 2119.4 | 2117.3 | |
| I-1451 | A | | | | 2144.8545 | 2147.3 | 2145.1 | |
| I-1452 | A | C | ++ | | 2126.9927 | 2125.9 | | A |
| I-1453 | A | | | | 2173.8698 | 2176.2 | 2174.4 | |
| I-1454 | A | | ++ | | 2172.8858 | 2175.2 | 2173.2 | |
| I-1455 | A | | | | 2172.8858 | 2176.1 | 2174.1 | |
| I-1456 | B | | +++ | | 2201.9011 | 2005.1 | 2002.6 | |
| I-1457 | B | | ++ | | 2123.8905 | 1064.3 | 2124.8 | |
| I-1458 | A | | ++ | | 2121.8749 | 2124.4 | 2122.9 | |
| I-1459 | B | | | | 2109.8749 | 2111.0 | 2111.0 | |
| I-1460 | A | | | | 2137.8698 | 2139.1 | 2139.1 | |
| I-1461 | A | | | | 2158.8701 | 2160.1 | 2160.1 | |
| I-1462 | B | | | | 2107.8592 | 2109.0 | 2109.0 | |
| I-1463 | A | D | ++ | | 2140.9356 | 2143.8 | 2141.5 | |
| I-1464 | A | | ++ | | 2124.9771 | 1064.6 | | |
| I-1465 | A | D | ++ | | 2082.9301 | 2085.8 | 2084.2 | |
| I-1466 | A | D | ++ | | 2110.9614 | 1057.6 | | |
| I-1467 | A | | ++ | | 2154.9512 | 2156.5 | | |
| I-1468 | A | | +++ | | 2096.9458 | 2099.8 | | |
| I-1469 | A | | | | 2186.9014 | | | |
| I-1470 | B | | | | 2186.9014 | | | |
| I-1471 | B | | | | 2123.8905 | | | |
| I-1472 | A | | | | 2263.0006 | 2263.8 | 2262.1 | |
| I-1473 | A | | | | 2305.0476 | 2305.9 | 2304.4 | |
| I-1474 | A | | | | 2402.1003 | 1201.6 | 1099.7 | |
| I-1475 | A | | | | 2293.0112 | 2293.9 | 2292.4 | |
| I-1476 | A | | | | 2390.0639 | 2391.2 | 2389.7 | |
| I-1477 | A | | | | 2289.0163 | 2289.9 | 2288.4 | |
| I-1478 | A | | | | 2261.0213 | 2261.9 | 2260.2 | |
| I-1479 | A | | | | 2303.0683 | 2303.9 | 2302.5 | |
| I-1480 | A | | | | 2400.1210 | 2401.1 | 2399.6 | |
| I-1481 | A | | | | 2291.0319 | 2291.8 | 2290.4 | |
| I-1482 | A | | | | 2388.0847 | 2389.2 | 2387.7 | |
| I-1483 | A | | | | 2287.0370 | 2287.9 | 2285.7 | |
| I-1484 | E | | | | 2032.8739 | 2033.6 | 2031.4 | |
| I-1485 | E | D | | | 2036.9052 | 2037.4 | 2035.7 | A |
| I-1486 | B | | | | 2060.9052 | 2062.7 | 2060.3 | |
| I-1487 | B | | | | 2060.9052 | 1031.9 | 1030.0 | |
| I-1488 | A | | | + | 2074.9209 | 2077.0 | 2074.9 | |
| I-1489 | A | | | | 2074.9209 | 2076.9 | 2074.9 | |
| I-1490 | B | | | | 2088.9365 | 2091.1 | 2088.6 | |
| I-1491 | A | | | | 2088.9365 | 2091.0 | 2088.7 | |
| I-1492 | B | | | | 2004.8426 | 2006.7 | 2004.4 | |
| I-1493 | E | | | | 2018.8583 | 1011.0 | 1009.0 | |
| I-1494 | A | | | | 2032.8739 | 2034.9 | 2032.9 | |
| I-1495 | A | | | | 2032.8739 | 1017.8 | 1015.9 | |
| I-1496 | A | | +++ | +++ | 2064.9365 | 2067.2 | 2065.6 | A |
| I-1497 | A | | +++ | +++ | 2078.9522 | 2081.1 | 2079.6 | A |
| I-1498 | C | | ++ | + | 2092.9678 | 2095.1 | 2092.5 | A |
| I-1499 | A | | +++ | | 2008.8739 | 2010.8 | 2008.8 | A |
| I-1500 | A | | +++ | | 2022.8896 | 2024.9 | 2023.0 | A |
| I-1501 | A | | | | 2036.9052 | 1019.9 | 1017.9 | A |
| I-1502 | A | | | | 2069.8540 | 2072.9 | 2070.7 | |
| I-1503 | A | | | | 2055.8384 | 2058.9 | 2057.2 | |
| I-1504 | A | | | | 2095.8697 | 1049.9 | 1048.0 | |
| I-1505 | A | | | + | 2055.8384 | 1030.4 | 1028.0 | |
| I-1506 | A | | | + | 2041.8227 | 1022.8 | 1020.7 | |
| I-1507 | A | | | | 2081.8540 | 2085.0 | 2083.3 | |
| I-1508 | A | | | + | 2009.8773 | 1006.6 | 1004.6 | |
| I-1509 | A | | | | 2009.8773 | 2012.0 | 2010.3 | |
| I-1510 | C | | | | 1995.8617 | 988.2 | 986.1 | |
| I-1511 | C | | | | 2035.8930 | 1008.6 | 1006.8 | |
| I-1512 | C | | | | 2009.8773 | 995.3 | 993.3 | |
| I-1513 | C | | | | 1995.8617 | 988.2 | 986.1 | |
| I-1514 | C | | | | 2035.8930 | 2038.6 | | |
| I-1515 | C | | | | 2049.9086 | 1015.0 | 1013.3 | |
| I-1516 | C | | | | 2035.8930 | 1008.3 | 1006.4 | |
| I-1517 | C | | | | 2035.8930 | 1008.3 | 1006.4 | |
| I-1518 | A | | | | 2021.8773 | 1012.7 | 2022.9 | |
| I-1519 | A | | | | 2159.8541 | 2162.0 | 2160.6 | |
| I-1520 | A | | | | 2160.8494 | 2163.2 | 2160.9 | |
| I-1521 | A | | | | 2111.8541 | 2113.2 | 2111.1 | |
| I-1522 | A | | | | 2097.8385 | 2099.7 | 2097.3 | |
| I-1523 | A | | | | 2114.9927 | 1061.4 | 2119.7 | A |
| I-1524 | B | | | | 2141.0084 | 1075.5 | 1073.5 | A |
| I-1525 | A | | | | 5122.6777 | 1025.7 | | |
| I-1526 | A | | | | 7764.2506 | 1554.1 | | |
| I-1527 | A | | | + | 2057.9040 | 2060.3 | 2057.8 | |
| I-1528 | A | | | + | 2043.8884 | 2046.3 | 2044.2 | |
| I-1529 | A | | | | 2083.9197 | 1043.6 | 1041.4 | |
| I-1530 | A | | | + | 2043.8884 | 2046.7 | 2044.2 | |
| I-1531 | A | | | + | 2029.8727 | 2032.6 | 2030.3 | |
| I-1532 | B | | | | 2085.9086 | 2088.8 | 2085.5 | |
| I-1533 | C | | | | 2085.9086 | 2088.9 | 2086.8 | |
| I-1534 | A | | | | 2071.8930 | 2074.8 | 2072.9 | |
| I-1535 | B | | | | 2071.8930 | 2074.9 | 2073.2 | |
| I-1536 | B | | | | 2073.9587 | 2075.6 | 2073.3 | |
| I-1537 | A | | | + | 2027.8679 | 2030.3 | 2029.1 | |
| I-1538 | A | | | | 2027.8679 | 2030.3 | 2029.5 | |
| I-1539 | A | | | + | 2013.8523 | 2016.1 | 2013.9 | |
| I-1540 | A | | | | 2013.8523 | 2016.2 | 2013.7 | |
| I-1541 | A | | | | 2015.9179 | 2017.3 | 2015.3 | |
| I-1542 | A | | | | 2015.9179 | 2017.4 | 2015.3 | |
| I-1543 | A | | | | 2001.9023 | 2003.4 | 2001.1 | |
| I-1544 | A | | | | 2001.9023 | 2003.4 | 2001.2 | |
| I-1545 | C | | | | 2011.9430 | 1006.8 | 1004.4 | |
| I-1546 | A | | | | 1997.9274 | 1999.4 | 1997.2 | |
| I-1547 | A | | | | 1997.9274 | 1999.5 | 1996.8 | |
| I-1548 | A | | | | 2019.8651 | 2022.2 | 2020.9 | A |
| I-1549 | B | | | +++ | 1963.8025 | 1967.2 | 1965.2 | A |
| I-1550 | A | | | +++ | 2005.8494 | 2008.6 | 2006.3 | A |
| I-1551 | A | | | +++ | 1991.8338 | 1994.3 | 1992.1 | A |
| I-1552 | A | | | ++ | 2005.8494 | 2008.7 | 2006.5 | A |
| I-1553 | A | | | +++ | 2039.8338 | 2042.6 | 2039.5 | A |
| I-1554 | A | | | | 2031.8651 | 2034.2 | 2032.6 | A |
| I-1555 | A | | | | 2045.8807 | 2048.0 | 2046.0 | A |
| I-1556 | A | | | | 2115.8651 | 2118.1 | 2116.6 | A |
| I-1557 | C | | | | 2018.8810 | 2021.2 | 2019.0 | A |
| I-1558 | D | | | | 1991.8701 | 1994.1 | 1991.5 | A |
| I-1559 | A | | | | 2001.8181 | 2007.2 | 2005.2 | A |
| I-1560 | A | | | + | 2023.8930 | 2026.4 | 2024.5 | |
| I-1561 | A | | | + | 2023.8930 | 1013.8 | 2024.4 | |
| I-1562 | A | | | + | 2037.9086 | 2040.4 | 2038.5 | |
| I-1563 | A | | | | 2037.9086 | 2041.7 | 2039.4 | |
| I-1564 | A | | | + | 2035.8930 | 2038.3 | 2036.9 | |
| I-1565 | A | | | + | 2035.8930 | 1019.8 | 1017.4 | |
| I-1566 | A | | | | 2049.9086 | 1026.8 | | |
| I-1567 | A | | | | 2049.9086 | 2053.1 | 2049.8 | |
| I-1568 | A | | | + | 2063.9243 | 2066.6 | 2064.7 | |
| I-1569 | A | | | + | 2063.9243 | 2066.6 | 2064.4 | |
| I-1570 | A | | | | 2079.9192 | 1041.8 | 1039.7 | |
| I-1571 | A | | | | 2079.9192 | 1041.8 | | |
| I-1572 | A | | | + | 2055.8651 | 2058.6 | 2056.0 | |
| I-1573 | A | | | + | 2055.8651 | 2058.5 | 2056.4 | |
| I-1574 | A | | | + | 2069.8807 | 2072.6 | 2070.5 | |
| I-1575 | A | | | + | 2069.8807 | 2072.5 | 2070.0 | |
| I-1576 | A | | | | 2069.8807 | 1065.7 | 1063.7 | |
| I-1577 | A | | | + | 2067.8651 | 2070.6 | 2068.7 | |
| I-1578 | A | | | + | 2067.8651 | 2070.5 | 2068.6 | |
| I-1579 | A | | | + | 2081.8807 | 1042.8 | 1040.6 | |
| I-1580 | A | | | + | 2095.8964 | 2098.6 | 2096.0 | |
| I-1581 | A | | | + | 2111.8913 | 2114.9 | 2112.8 | |
| I-1582 | A | | | | 2015.8338 | 1009.8 | 1007.9 | |
| I-1583 | A | | | | 2015.8338 | 1009.8 | 1007.8 | |
| I-1584 | A | | | | 2029.8494 | 2032.2 | 2030.4 | |
| I-1585 | A | | | | 2029.8494 | 2032.7 | 2030.3 | |
| I-1586 | A | | | | 2027.8338 | 2030.4 | 2027.9 | |
| I-1587 | A | | | | 2027.8338 | 2030.5 | 2028.1 | |
| I-1588 | A | | | | 2041.8494 | 2044.6 | 2042.3 | |
| I-1589 | A | | | | 2041.8494 | 1022.8 | 1020.7 | |
| I-1590 | A | | | | 2055.8651 | 2058.6 | 2056.4 | |
| I-1591 | A | | | | 2055.8651 | 1029.8 | 1027.9 | |
| I-1592 | A | | | | 2071.8600 | 1037.8 | 1035.3 | |
| I-1593 | A | | | | 2071.8600 | 2074.5 | 2072.1 | |
| I-1594 | A | | | | 1927.9396 | 1929.5 | 1927.3 | |
| I-1595 | A | | | | 1927.9396 | 1929.5 | 1927.4 | |
| I-1596 | B | | | | 1961.9007 | 1963.8 | 1961.6 | |
| I-1597 | B | | | | 1961.9007 | 1964.0 | 1961.8 | |
| I-1598 | A | | | + | 1961.9007 | 1963.8 | 1962.0 | |
| I-1599 | A | | | | 1961.9007 | 1963.9 | 1962.0 | |
| I-1600 | A | | | | 1961.9007 | 982.4 | 980.6 | |
| I-1601 | A | | | | 1941.9553 | 1943.4 | 1941.3 | |
| I-1602 | B | | | | 2005.8502 | 2008.4 | 2005.8 | |
| I-1603 | C | | | | 2005.8502 | 1004.7 | 2006.7 | |
| I-1604 | A | | | + | 2005.8502 | 2008.4 | 2006.0 | |
| I-1605 | A | | | | 2005.8502 | 1004.6 | 1002.8 | |
| I-1606 | A | | | | 2005.8502 | 1004.6 | 1002.6 | |
| I-1607 | A | | | | 2005.8502 | 1004.7 | 1002.8 | |
| I-1608 | A | | | | 1995.9270 | 1997.4 | 1995.3 | |
| I-1609 | A | | | | 1995.9270 | 1997.5 | 1995.2 | |
| I-1610 | A | | | | 1995.9270 | 1997.5 | 1995.2 | |
| I-1611 | A | | | | 1995.9270 | 1997.4 | 1995.3 | |
| I-1612 | A | | | | 2011.8066 | 1007.7 | 1005.7 | |
| I-1613 | A | | | | 2011.8066 | 1007.7 | 1005.9 | |
| I-1614 | A | | | | 2001.8834 | 2003.3 | 2001.3 | |
| I-1615 | A | | | | 2001.8834 | 2003.4 | 2001.1 | |
| I-1616 | A | | | | 2112.9771 | 2115.4 | 2113.6 | A |
| I-1617 | A | | | | 2138.9927 | 1071.2 | 1069.3 | A |
| I-1618 | B | | | | 2141.0084 | 1072.2 | 1070.1 | A |
| I-1619 | A | | | | 2170.9825 | 1087.2 | 1085.7 | A |
| I-1620 | A | | | | 2098.9614 | 2101.0 | 2099.3 | A |
| I-1621 | A | | | | 2124.9771 | 1064.1 | 1061.9 | A |
| I-1622 | A | | | | 2126.9927 | 2129.2 | 2127.4 | A |
| I-1623 | A | | | | 2156.9669 | 2160.0 | 2157.9 | A |
| I-1624 | A | | | | 2126.9927 | 1065.0 | 1063.0 | A |
| I-1625 | A | | | | 2112.9771 | 2115.5 | 2113.3 | A |
| I-1626 | A | | | | 2098.9614 | 1051.2 | 1049.3 | A |
| I-1627 | A | | | | 2084.9458 | 2087.4 | 2085.5 | A |
| I-1628 | A | | | | 2194.0097 | 2195.6 | 2193.5 | |
| I-1629 | A | | | | 2194.0097 | 2195.6 | 2193.4 | |
| I-1630 | A | | | | 2136.8654 | 2139.4 | 2137.3 | |
| I-1631 | B | | | | 2123.8701 | 1063.8 | 1062.0 | |
| I-1632 | A | | | | 2136.8654 | 1070.2 | | |
| I-1633 | B | | | | 2123.8701 | 1063.8 | 1061.7 | |
| I-1634 | A | | | | 2139.8651 | 1071.7 | 1069.7 | |
| I-1635 | E | | | | 2127.8287 | 1814.1 | 1811.7 | |
| I-1636 | A | | | | 2138.8810 | 2141.5 | 2138.8 | |
| I-1637 | B | | | | 2125.8858 | 2128.7 | 2126.2 | |
| I-1638 | A | | | + | 2138.8810 | 2041.9 | 2039.3 | |
| I-1639 | B | | | + | 2125.8858 | 1064.8 | | |
| I-1640 | E | | | | 2199.8804 | 1085.8 | 1084.0 | |
| I-1641 | A | | | | 2170.9881 | 2173.0 | 2171.0 | |
| I-1642 | A | | | | 2170.9881 | 2172.9 | 2171.4 | |
| I-1643 | A | | | | 2128.9412 | 2130.9 | 2128.7 | |
| I-1644 | A | | | | 2128.9412 | 1065.9 | 1064.0 | |
| I-1645 | A | | | | 2137.0271 | 2138.5 | 2136.5 | |
| I-1646 | A | | | | 2137.0271 | 2138.5 | 2136.4 | |
| I-1647 | A | | | | 2094.9801 | 2096.4 | 2094.4 | |
| I-1648 | A | | | | 2023.9430 | 2025.3 | 2023.4 | |
| I-1649 | A | | | | 2023.9430 | 2025.4 | 2023.3 | |
| I-1650 | A | | | | 2155.0177 | 2156.6 | 2154.4 | |
| I-1651 | A | | | | 2155.0177 | 2156.5 | 2154.1 | |
| I-1652 | A | | | | 2112.9707 | 2114.4 | 2112.6 | |
| I-1653 | A | | | | 2041.9336 | 2043.3 | 2041.4 | |
| I-1654 | B | | | + | 2205.0145 | 2206.4 | 2204.3 | |
| I-1655 | B | | | + | 2205.0145 | 2206.5 | 2204.2 | |
| I-1656 | A | | | + | 2162.9675 | 2164.6 | 2162.3 | |
| I-1657 | A | | | + | 2162.9675 | 2164.5 | 2162.1 | |
| I-1658 | A | | | + | 2091.9304 | 2093.3 | 2091.3 | |
| I-1659 | A | | | + | 2091.9304 | 2093.4 | 2091.2 | |
| I-1660 | A | | | | 2202.0172 | 2203.4 | 2201.3 | |
| I-1661 | C | | | | 2202.0172 | 1102.3 | 1100.3 | |
| I-1662 | B | | | | 2188.0016 | 2189.4 | 2187.3 | |
| I-1663 | D | | | | 2188.0016 | 1095.3 | 1093.4 | |
| I-1664 | A | | | | 2264.0329 | 2265.5 | 2263.3 | |
| I-1665 | B | | | | 2264.0329 | 2265.5 | 2263.7 | |
| I-1666 | A | | | | 2224.0703 | 2225.6 | 2223.5 | |
| I-1667 | B | | | | 2242.0234 | 2243.5 | 2241.4 | |
| I-1668 | A | | | | 2242.0234 | 1122.2 | 1120.4 | |
| I-1669 | A | | | | 2242.0234 | 2243.5 | 2241.6 | |
| I-1670 | A | | | | 2203.0125 | 2204.5 | 2202.4 | |
| I-1671 | C | | | | 2203.0125 | 2204.5 | 2202.4 | |
| I-1672 | A | | | | 2229.0281 | 2230.5 | 2228.5 | |
| I-1673 | A | | | | 2130.9512 | 1067.1 | 1065.0 | A |
| I-1674 | C | | | | 2129.9672 | 2132.4 | 2130.0 | A |
| I-1675 | C | | | | 2102.9563 | 1053.0 | 1051.2 | A |
| I-1676 | C | | | | 2144.9669 | 1074.2 | 1072.3 | A |
| I-1677 | C | | | | 2143.9829 | 1073.6 | 1071.7 | A |
| I-1678 | C | | | | 2116.9720 | 1060.1 | 1058.1 | A |
| I-1679 | B | | | | 2158.9825 | 1081.1 | 1079.3 | A |
| I-1680 | A | | | | 2126.9 | 2128.2 | 2125.8 | |
| I-1681 | A | | | | 2099.9 | 2101.3 | 2098.9 | |
| I-1682 | C | | | | 2113.9 | 2115.1 | 2113.0 | |
| I-1683 | C | | | | 2099.9 | 2101.2 | 2099.0 | |
| I-1684 | C | | | | 2113.9 | 2115.2 | 2113.3 | |
| I-1685 | C | | | | 2113.9 | 2115.1 | 2113.8 | |
| I-1686 | B | | | | 2113.9 | 2115.2 | 2113.1 | |
| I-1687 | C | | | | 2119.9 | 2121.2 | 2119.3 | |
| I-1688 | A | | | | 2211.0 | 1107.1 | 1105.3 | A |
| I-1689 | A | | | + | 2197.0 | 2199.0 | 2197.3 | A |
| I-1690 | A | | | + | 2225.0 | 2228.3 | 2226.0 | A |
| I-1691 | A | | | | 2211.0 | 2212.9 | 2210.3 | A |
| I-1692 | A | | | + | 2183.0 | 2185.2 | 2183.6 | A |
| I-1693 | A | | | + | 2168.9 | 2170.6 | 2169.0 | A |
| I-1694 | A | | | | 2195.0 | 1099.0 | 1097.2 | A |
| I-1695 | A | | | + | 2223.0 | 2224.9 | 2222.8 | A |
| I-1696 | A | | | | 2209.0 | 2210.7 | 2208.7 | |
| I-1697 | A | | | | 1985.9 | 1987.7 | | A |
| I-1698 | A | | | | 1971.9 | 1973.8 | 1971.6 | A |
| I-1699 | A | | | | 1985.9 | 1987.8 | 1985.5 | A |
| I-1700 | A | | | | 1971.9 | 1973.7 | 1971.5 | A |
| I-1701 | A | | | + | 2620.1 | 1311.9 | 1309.9 | |
| I-1702 | A | | | + | 3896.9 | 1950.3 | 1948.7 | |
| I-1703 | A | C | | + | 2762.2 | 1382.8 | 1381.0 | |
| I-1704 | A | D | | + | 4039.0 | 2021.3 | 2019.2 | |
| I-1705 | A | C | | + | 2833.2 | 1418.4 | 1416.2 | |
| I-1706 | A | D | | + | 4110.0 | 2056.9 | 2054.8 | |
| I-1707 | A | | | + | 2751.3 | 1377.5 | 1375.3 | |
| I-1708 | A | D | | + | 4028.0 | 2016.0 | 2014.2 | |
| I-1709 | A | | ++ | | 2004.9 | 2006.2 | 2004.0 | |
| I-1710 | A | | | + | 2165.9 | 1084.4 | 1082.3 | |
| I-1711 | A | | | | 2179.9 | 2181.6 | 2179.9 | |
| I-1712 | A | C | | + | 2163.9 | 2165.4 | 2162.6 | |
| I-1713 | A | C | | + | 2177.9 | 2180.7 | 2178.1 | |
| I-1714 | A | C | | + | 2191.9 | 2193.6 | 2191.6 | |
| I-1715 | A | | | | 2213.9 | 2215.6 | 2212.8 | |
| I-1716 | A | | | | 2191.8 | 2193.5 | | |
| I-1717 | A | B | | + | 2264.9 | 2266.5 | 2264.6 | |
| I-1718 | A | | | | 2204.9 | 2205.6 | 2203.5 | |
| I-1719 | A | | | | 2215.9 | 2216.9 | 2214.6 | |
| I-1720 | A | | | | 2240.9 | 2141.8 | 2140.2 | |
| I-1721 | A | | | | 2242.9 | 2244.6 | 2243.1 | |
| I-1722 | A | | | | 2201.9 | 2204.3 | | |
| I-1723 | A | C | | + | 2164.9 | 2266.5 | 2263.7 | |
| I-1724 | A | | | +++ | 2178.9 | 2180.6 | 2179.2 | |
| I-1725 | A | C | | +++ | 2162.9 | 2164.6 | | |
| I-1726 | A | | | +++ | 2176.9 | 2179.5 | 2177.7 | |
| I-1727 | A | | | +++ | 2190.9 | 2193.8 | 2191.1 | |
| I-1728 | B | | | | 2212.9 | 2215.6 | 2213.5 | |
| I-1729 | A | | | | 2190.8 | 2193.4 | 2191.6 | |
| I-1730 | A | | | +++ | 2263.9 | 2265.5 | 2264.5 | |
| I-1731 | A | | | | 2203.9 | 2205.7 | | |
| I-1732 | A | | | +++ | 2239.9 | 2241.5 | 2239.2 | |
| I-1733 | A | | | +++ | 2241.9 | 2243.7 | 2241.7 | |
| I-1734 | A | | | | 2200.9 | 2203.6 | 2201.6 | |
| I-1735 | C | | | | 2084.0 | 2086.1 | 2084.4 | |
| I-1736 | A | | | + | 2084.0 | 2086.2 | 2084.7 | |
| I-1737 | A | | | | 2193.0 | 2195.1 | 2193.8 | |
| I-1738 | A | | | | 2207.0 | 2209.1 | 2207.8 | |
| I-1739 | A | | | | 2253.0 | 2255.1 | 2253.6 | |
| I-1740 | A | | | + | 2203.0 | 2205.4 | 2203.5 | |
| I-1741 | A | | | | 2217.0 | 2217.8 | 2215.8 | |
| I-1742 | A | | | ++ | 2217.0 | 2219.1 | 2217.7 | |
| I-1743 | A | | | ++ | 2217.0 | 2219.2 | 2217.8 | |
| I-1744 | C | | | | 2170.0 | 2172.1 | 2170.4 | |
| I-1745 | B | | | | 2232.0 | 1117.7 | 1116.2 | |
| I-1746 | D | | | | 2198.0 | 1100.6 | 1198.9 | |
| I-1747 | A | | | | 2169.0 | 2171.2 | 2169.6 | |
| I-1748 | C | | | | 2169.0 | 2171.1 | 2169.1 | |
| I-1749 | A | | | | 2183.0 | 2182.8 | 2180.9 | |
| I-1750 | B | | | | 2197.1 | 1100.0 | 1098.5 | |
| I-1751 | A | | | | 2242.0 | 1122.9 | 1120.9 | |
| I-1752 | A | C | | + | 2229.0 | 1116.3 | 1114.4 | |
| I-1753 | A | | | +++ | 2211.0 | 2213.1 | 2212.0 | |
| I-1754 | A | | | +++ | 2189.0 | 2191.2 | | |
| I-1755 | A | | | +++ | 2190.0 | 2192.2 | 2190.6 | |
| I-1756 | A | | | | 2190.0 | 1096.4 | 1094.6 | |
| I-1757 | A | | | | 1995.9 | | | |
| I-1758 | A | | | | 2009.9 | | | |
| I-1759 | B | | | | 2066.9 | | | |
| I-1760 | A | | | | 2009.9 | | | |
| I-1761 | A | | | | 2023.9 | | | |
| I-1762 | A | | | | 2080.9 | | | |
| I-1763 | B | | | | 2207.0 | 1104.9 | 1103.4 | |
| I-1764 | A | | | | 2207.0 | 2209.2 | 2207.7 | |
| I-1765 | A | | | | 2207.0 | 1105.1 | 1103.9 | |
| I-1766 | A | D | | +++ | 2187.9 | | | |
| I-1767 | A | | | | 2296.9 | | | |
| I-1768 | A | | | | 2285.0 | | | |
| I-1769 | B | D | | + | 2313.0 | | | |
| I-1770 | B | D | | + | 2301.0 | | | |
| I-1771 | A | | | | 2286.9 | | | |
| I-1772 | A | D | | + | 2275.0 | | | |
| I-1773 | A | D | | + | 2298.9 | | | |
| I-1774 | A | C | | + | 2287.0 | | | |
| I-1775 | A | | | | 2287.0 | | | |
| I-1776 | A | | | | 2346.9 | | | |
| I-1777 | B | D | | + | 2335.0 | | | |
| I-1778 | A | | | | 2066.9 | | | |
| I-1779 | A | | | | 1981.8 | | | |
| I-1780 | A | | | | 1981.8 | 992.3 | 990.3 | |
| I-1781 | A | | | | 1995.9 | | | |
| I-1782 | A | | | | 2052.9 | | | |
| I-1783 | A | | | | 2211.0 | 2212.7 | 2210.0 | |
| I-1784 | A | C | | | 2227.0 | 2229.1 | 2227.0 | |
| I-1785 | A | | | | 2211.0 | 2212.7 | 2210.6 | |
| I-1786 | A | C | | | 2031.9 | 2033.8 | 2031.7 | |
| I-1787 | A | | ++ | + | 2569.1 | 1286.3 | 1284.3 | |
| I-1788 | A | | ++ | + | 2833.2 | 1418.3 | 1416.3 | |
| I-1789 | A | | ++ | + | 2653.2 | 1328.3 | 1326.3 | |
| I-1790 | A | | ++ | + | 2917.3 | 1460.4 | 1458.3 | |
| I-1791 | E | | + | + | 2793.3 | 1398.4 | | |
| I-1792 | E | | + | + | 3057.5 | 1530.5 | | |
| I-1793 | B | | | | 2080.9 | 2082.9 | 2080.6 | |
| I-1794 | C | | | | 2038.9 | 2040.8 | 2039.0 | |
| I-1795 | C | | | | 2066.9 | 2068.3 | 2065.7 | |
| I-1796 | A | | | | 2094.9 | 2097.0 | 2095.5 | |
| I-1797 | A | D | | | 2052.9 | 2054.8 | | |
| I-1798 | A | C | | | 2080.9 | 1042.0 | | |
| I-1799 | B | | | | 2094.9 | 2096.2 | 2094.5 | |
| I-1800 | C | | | | 2052.9 | 2055.9 | 2053.6 | |
| I-1801 | E | | | | 2080.9 | 2083.7 | 2082.1 | |
| I-1802 | B | | | | 2094.9 | 2096.8 | | |
| I-1803 | C | | | | 2052.9 | 2055.0 | 2053.4 | |
| I-1804 | C | | | | 2080.9 | 2082.9 | 2080.9 | |
| I-1805 | A | C | | | 2324.0 | 1163.5 | 1161.3 | |
| I-1806 | A | C | | | 2289.0 | 1146.0 | 1144.1 | |
| I-1807 | A | D | | | 2199.0 | 2200.8 | 2198.7 | |
| I-1808 | A | D | | | 2245.0 | 1124.0 | 1121.6 | |
| I-1809 | A | B | | | 2097.0 | 2098.0 | 2095.8 | |
| I-1810 | A | C | | | 2097.0 | 2098.0 | 2095.9 | |
| I-1811 | B | | | | 2092.9 | 2095.1 | 2093.0 | |
| I-1812 | A | D | | | 2125.0 | 2126.1 | 2124.0 | |
| I-1813 | A | C | | | 2125.0 | 2126.1 | 2124.0 | |
| I-1814 | A | D | | | 2125.0 | 2126.1 | 2123.8 | |
| I-1815 | A | | ++ | | 2698.3 | 1350.2 | 1348.2 | |
| I-1816 | A | | ++ | | 2698.3 | 1350.2 | 1348.2 | |
| I-1817 | A | C | ++ | | 2754.3 | 1378.2 | 1376.2 | |
| I-1818 | A | | | | 2726.3 | 1364.3 | 1362.4 | |
| I-1819 | A | | | | 2726.3 | 1364.3 | 1362.2 | |
| I-1820 | A | B | ++ | | 2726.3 | 1364.2 | 1362.2 | |
| I-1821 | A | | ++ | | 2726.3 | 1364.2 | 1362.3 | |
| I-1822 | A | | ++ | | 2698.3 | 1350.2 | 1348.3 | |
| I-1823 | B | | ++ | | 2698.3 | 1350.6 | 1348.5 | |
| I-1824 | A | | | | 2108.9 | 2110.9 | 2109.2 | |
| I-1825 | A | | | | 2066.9 | 2068.8 | 2066.1 | |
| I-1826 | A | | | | 2094.9 | 2096.8 | 2095.1 | |
| I-1827 | A | | | | 2108.9 | 2110.9 | 2109.5 | |
| I-1828 | A | | | | 2066.9 | 2069.7 | 2066.8 | |
| I-1829 | A | | | | 2094.9 | 2096.9 | 2095.0 | |
| I-1830 | A | | | | 2108.9 | 2110.9 | 2107.7 | |
| I-1831 | A | | | | 2066.9 | 2068.8 | 2066.6 | |
| I-1832 | A | | | | 2094.9 | 2096.8 | 2094.9 | |
| I-1833 | A | | | | 2123.0 | 2124.9 | | |
| I-1834 | A | | | | 2080.9 | 2082.8 | 2070.7 | |
| I-1835 | A | | | | 2108.9 | 2110.9 | 2109.3 | |
| I-1836 | A | | | | 2135.0 | 1069.0 | 2135.0 | |
| I-1837 | B | | | | 2120.9 | 2123.2 | 2121.5 | |
| I-1838 | A | | | | 2135.0 | 1069.0 | 1066.8 | |
| I-1839 | A | | | | 2092.9 | 1048.0 | 1046.1 | |
| I-1840 | B | | | | 2120.9 | 1062.0 | 1060.1 | |
| I-1841 | A | | | | 2079.9 | 2081.0 | 2079.0 | |
| I-1842 | A | | | | 2079.9 | 2081.4 | 2079.2 | |
| I-1843 | A | | | | 2177.0 | 2178.1 | 2175.5 | |
| I-1844 | A | | | | 2177.0 | 2178.3 | 2176.0 | |
| I-1845 | A | | | | 2177.0 | 2178.0 | 2175.9 | |
| I-1846 | A | | | | 2166.9 | 2168.2 | 2165.7 | |
| I-1847 | A | | | | 2179.0 | 2180.1 | 2177.9 | |
| I-1848 | A | | | | 2179.0 | 2180.2 | 2177.8 | |
| I-1849 | A | | | | 2179.0 | 2180.1 | 2178.1 | |
| I-1850 | A | | | | 2122.0 | 2123.1 | 2120.9 | |
| I-1851 | A | | | | 2122.0 | 2123.1 | 2120.9 | |
| I-1852 | A | | | | 2107.9 | 2109.0 | 2106.9 | |
| I-1853 | A | | | | 2109.9 | 2111.0 | 2108.9 | |
| I-1854 | A | | | | 2219.0 | 2220.1 | 2218.7 | |
| I-1855 | A | | | + | 2205.0 | 2206.1 | 2203.6 | |
| I-1856 | A | | | | 2207.0 | 2208.1 | 2205.8 | |
| I-1857 | A | C | | ++ | 2005.9 | 2007.7 | 2006.2 | |
| I-1858 | A | C | | + | 2035.9 | 2037.6 | 2035.8 | |
| I-1859 | A | C | | + | 2035.9 | 2037.7 | 2036.4 | |
| I-1860 | A | C | | + | 2021.9 | 2023.8 | 2021.3 | |
| I-1861 | A | | | + | 2021.9 | 2023.6 | 2021.7 | |
| I-1862 | A | C | | + | 2021.9 | 2023.7 | 2021.9 | |
| I-1863 | A | | | + | 1991.8 | 1993.7 | 1991.9 | |
| I-1864 | A | | | +++ | 1991.8 | 1993.8 | 1992.3 | |
| I-1865 | A | | | + | 2021.9 | 2023.8 | 2021.9 | |
| I-1866 | A | | | ++ | 2021.9 | 2023.7 | 2022.3 | |
| I-1867 | A | | | | 2007.8 | 2009.6 | 2007.8 | |
| I-1868 | A | | | | 2007.8 | 2009.7 | | |
| I-1869 | A | | | + | 2007.8 | 2009.7 | 2007.9 | |
| I-1870 | A | | | + | 2007.8 | 2009.7 | 2008.2 | |
| I-1871 | A | | | | 2206.9 | | | A |
| I-1872 | A | | | | 2221.0 | 2222.1 | 2220.1 | A |
| I-1873 | A | D | | | 2262.0 | 2263.2 | 2261.4 | A |
| I-1874 | A | | | | 2205.0 | 2206.1 | 2204.1 | A |
| I-1875 | A | | | | 2283.0 | 2284.0 | 2282.1 | A |
| I-1876 | A | | | | 2163.0 | 2164.2 | 2162.6 | A |
| I-1877 | A | | | + | 2082.9 | 2084.1 | 2082.3 | A |
| I-1878 | A | | | + | 2096.9 | 2098.1 | 2096.2 | A |
| I-1879 | A | D | | +++ | 2138.0 | 2139.2 | 2136.9 | A |
| I-1880 | A | D | | + | 2081.0 | 2082.2 | 2080.4 | A |
| I-1881 | A | D | | + | 2158.9 | 1080.7 | 1078.8 | A |
| I-1882 | A | | | + | 2038.9 | 2040.0 | 2038.1 | A |
| I-1883 | A | | | | 2152.0 | 2157.8 | 2156.6 | |
| I-1884 | A | | | | 2152.0 | 2157.8 | 2156.0 | |
| I-1885 | A | | | | 2324.0 | | | A |
| I-1886 | A | | | | 2200.0 | 2202.3 | 2200.3 | A |
| I-1887 | A | | | | 2214.0 | 2216.0 | 2214.2 | A |
| I-1888 | A | | | | 2266.0 | 2068.0 | 2065.8 | A |
| I-1889 | A | | | | 2142.0 | 2144.1 | 2142.1 | A |
| I-1890 | A | | | | 2294.0 | | | A |
| I-1891 | A | | | | 2170.0 | 2171.9 | 2170.1 | A |
| I-1892 | A | | | | 2184.0 | 1093.5 | 1091.6 | A |
| I-1893 | A | | | | 2308.0 | 2310.0 | 2308.0 | A |
| I-1894 | A | | | | 2184.0 | 2186.0 | 2183.8 | A |
| I-1895 | A | | | | 2198.0 | 1100.5 | 1098.5 | A |
| I-1896 | A | | | | 2135.0 | 2136.9 | 2134.6 | |
| I-1897 | A | | | | 2092.9 | 2095.7 | 2093.8 | |
| I-1898 | A | | | | 2135.0 | 2138.2 | | |
| I-1899 | A | | | | 2092.9 | 2094.8 | | |
| I-1900 | B | | | | 2104.9 | 2106.8 | 2104.4 | |
| I-1901 | B | | | | 2147.0 | 2149.9 | | |
| I-1902 | B | | | | 2104.9 | 2107.1 | 2105.4 | |
| I-1903 | A | | | | 2147.0 | 2148.9 | 2147.1 | |
| I-1904 | A | | | | 2104.9 | 2107.2 | | |
| I-1905 | A | | | | 2147.0 | 2149.1 | 2147.3 | |
| I-1906 | A | | | | 2104.9 | 2107.8 | | |
| I-1907 | A | | | | 2066.9 | 2069.0 | 2067.4 | |
| I-1908 | A | | | | 2105.9 | 2108.2 | | |
| I-1909 | A | | | | 2122.9 | 2125.2 | 2123.1 | |
| I-1910 | A | | | | 2139.9 | 1071.9 | 1069.6 | |
| I-1911 | C | | | | 2139.9 | 1071.8 | 1070;1 | |
| I-1912 | A | | | | 2054.9 | 2056.3 | 2054.1 | |
| I-1913 | B | | | | 2094.0 | 2095.4 | 2093.3 | |
| I-1914 | A | | | | 2110.9 | 2112.4 | | |
| I-1915 | D | | | | 2127.9 | 2129.5 | 2126.9 | |
| I-1916 | B | | | | 2127.9 | 2129.8 | 2127.9 | |
| I-1917 | A | | | | 2689.2 | | | |
| I-1918 | A | | | | 2953.3 | | | |
| I-1919 | B | | | | 2885.4 | | | |
| I-1920 | B | | | | 3149.5 | | | |
| I-1921 | A | | | | 2175.0 | 2177;2 | | |
| I-1922 | A | | | | 2132.9 | 2136.1 | | |
| I-1923 | A | | | | 2092.9 | 2095.1 | 2093.1 | |
| I-1924 | A | | | | 2135.0 | 2136.9 | 2135.3 | |
| I-1925 | A | | | | 2120.9 | 1062.1 | 1060.1 | |
| I-1926 | A | | | | 2092.9 | 1048.1 | 1046.4 | |
| I-1927 | A | | | | 2241.0 | | | A |
| I-1928 | A | | | | 2229.0 | | | A |
| I-1929 | A | | | | 2235.0 | | | A |
| I-1930 | A | | | | 2269.0 | | | A |
| I-1931 | A | | | | 2257.0 | | | A |
| I-1932 | A | | | | 2263.0 | | | A |
| I-1933 | A | | | | 2340.0 | | | A |
| I-1934 | A | | | | 2346.0 | | | A |
| I-1935 | A | | | | 2109.0 | | | A |
| I-1936 | A | | | | 2149.0 | | | A |
| I-1937 | A | | | | 2123.0 | | | A |
| I-1938 | A | | | | 2095.0 | | | A |
| I-1939 | A | | | | 2219.0 | | | A |
| I-1940 | A | | | | 2192.0 | | | A |
| I-1941 | A | | | | 2232.1 | | | A |

| 1 | Description |
|---|---|
| I-608 | Ac-PL3-Asp-Leu-B5-Asp-Asp-dLys*3-Ala-Phe-GlnR*3-PyrS2-3Thi-BztA-Gln-NH2 |
| I-609 | Ac-PL3-Asp-Leu-B5-Asp-Asp-DGlnR*3-Ala-Phe-Lys*3-PyrS2-3Thi-BztA-Gln-NH2 |
| I-700 | Ac-PL3-Asp-Leu-B5-Asp-Asp-DGlnR*3-Ala-Phe-Lys*3-PyrS2-3Thi-BztA-Gln-NH2 |
| I-701 | Ac-PL3-Asp-Npg-B5-Asp-3COOHF-[ethylenediamine]GlnR-Ala-Phe-Leu-PyrS2-2F3MeF-BztA- |
| | GlnR-NH2 |
| I-702 | Ac-PL3-Asp-Npg-B5-Asp-3COOHF-[Me2ethylenediamine]GlnR-Ala-Phe-Leu-PyrS2-2F3MeF- |
| | BztA-GlnR-NH2 |
| I-703 | Ac-PL3-Asp-Npg-B5-Asp-3COOHF-[diaminopropane]GlnR-Ala-Phe-Leu-PyrS2-2F3MeF-BztA- |
| | GlnR-NH2 |
| I-704 | Ac-PL3-Asp-Npg-B5-Asp-3COOHF-[diaminopentane]GlnR-Ala-Phe-Leu-PyrS2-2F3MeF-BztA- |
| | GlnR-NH2 |
| I-705 | Ac-PL3-Asp-Npg-B5-Asp-3COOHF-[Me2diaminohexane]GlnR-Ala-Phe-Leu-PyrS2-2F3MeF- |
| | BztA-GlnR-NH2 |
| I-706 | Ac-PL3-Asp-Ile-B5-Asp-3COOHF-Aib-Ala-Phe-Lys*3-PyrS2-3Thi-BztA-GlnR*3-Ala-NH2 |
| I-707 | Ac-PL3-Asp-Chg-B5-Asp-3COOHF-Aib-Ala-Phe-Lys*3-PyrS2-3Thi-BztA-GlnR*3-Ala-NH2 |
| I-708 | Ac-PL3-Asp-DipA-B5-Asp-3COOHF-Aib-Ala-Phe-Lys*3-PyrS2-3Thi-BztA-GlnR*3-Ala-NH2 |
| I-709 | Ac-PL3-Asp-Npg-B5-Asp-3COOHF-Aib-Ala-Phe-TriAzLys*3-PyrS2-3Thi-BztA-sAla*3-Ala-NH2 |
| I-710 | Ac-PL3-Asp-Npg-B5-Asp-3COOHF-Aib-Ala-Phe-1MeK*3-PyrS2-3Thi-BztA-hGlnR*3-Ala-NH2 |
| I-711 | Ac-PL3-Asp-Npg-B5-Asp-3COOHF-Aib-Ala-Phe-1MeK*3-PyrS2-3Thi-BztA-GlnR*3-Ala-NH2 |
| I-712 | Ac-PL3-Asp-Npg-B5-Asp-3COOHF-Aib-Ala-Phe-Lys*3-PyrS2-3Thi-BztA-GlnR*3-NH2 |
| I-713 | Ac-PL3-Asp-Npg-B5-Asp-3COOHF-Aib-Ala-Phe-Lys*3-PyrS2-3Thi-BztA-GlnR*3-NH2 |
| I-714 | Ac-PL3-Asp-Npg-B5-Asp-3COOHF-Aib-Ala-Phe-Lys*3-PyrS2-3Thi-BztA-GlnR*3-Gly-NH2 |
| I-715 | Ac-PL3-Asp-Npg-B5-Asp-3COOHF-Aib-Ala-Phe-Lys*3-PyrS2-3Thi-BztA-GlnR*3-Leu-NH2 |
| I-716 | Ac-PL3-Asp-Npg-B5-Asp-3COOHF-Aib-Ala-Phe-Lys*3-PyrS2-3Thi-BztA-GlnR*3-Leu-NH2 |
| I-717 | Ac-PL3-Asp-Npg-B5-Asp-3COOHF-Aib-Ala-Phe-Lys*3-PyrS2-3Thi-BztA-GlnR*3-Npg-NH2 |
| I-718 | Ac-PL3-Asp-Npg-B5-Asp-3COOHF-Aib-Ala-Phe-Lys*3-PyrS2-3Thi-BztA-GlnR*3-Npg-NH2 |
| I-719 | Ac-PL3-Asp-Npg-B5-Asp-3COOHF-Aib-Ala-Phe-Lys*3-PyrS2-3Thi-BztA-GlnR*3-Pro-NH2 |
| I-720 | Ac-PL3-Asp-Npg-B5-Asp-3COOHF-Aib-Ala-Phe-Lys*3-PyrS2-3Thi-BztA-GlnR*3-dPro-NH2 |
| I-721 | Ac-PL3-Asp-Npg-B5-Asp-3COOHF-Aib-Ala-Phe-Lys*3-PyrS2-3Thi-BztA-GlnR*3-Ser-NH2 |
| I-722 | Ac-PL3-Asp-Npg-B5-Asp-3COOHF-Aib-Ala-Phe-Lys*3-PyrS2-3Thi-BztA-GlnR*3-Phe-NH2 |
| I-723 | Ac-PL3-Asp-Npg-B5-Asp-3COOHF-Aib-Ala-Phe-Lys*3-PyrS2-3Thi-BztA-GlnR*3-Asn-NH2 |
| I-724 | Ac-PL3-Asp-Npg-B5-Asp-3COOHF-Aib-Ala-Phe-Lys*3-PyrS2-3Thi-BztA-GlnR*3-Gln-NH2 |
| I-725 | Ac-PL3-Asp-Npg-B5-Asp-3COOHF-Aib-Ala-Phe-Lys*3-PyrS2-3Thi-BztA-GlnR*3-Trp-NH2 |
| I-726 | Ac-PL3-Asp-Npg-B5-Asp-3COOHF-Aib-Ala-Phe-Lys*3-PyrS2-3Thi-BztA-GlnR*3-Trp-NH2 |
| I-727 | Ac-PL3-Asp-Npg-B5-Asp-3COOHF-Aib-Ala-Phe-Lys*3-PyrS2-3Thi-BztA-GlnR*3-Tyr-NH2 |
| I-728 | Ac-PL3-Asp-Npg-B5-Asp-3COOHF-Aib-Ala-Phe-Lys*3-PyrS2-3Thi-BztA-GlnR*3-Tyr-NH2 |
| I-729 | Ac-PL3-Asp-Ile-B5-Asp-3COOHF-Phe-Ala-Phe-Lys*3-PyrS2-3Thi-BztA-GlnR*3-Ala-NH2 |
| I-730 | Ac-PL3-Asp-Ile-B5-Asp-3COOHF-CyLeu-Ala-Phe-Lys*3-PyrS2-3Thi-BztA-GlnR*3-Ala-NH2 |
| I-731 | Ac-PL3-Asp-Ile-B5-Asp-3COOHF-Cha-Ala-Phe-Lys*3-PyrS2-3Thi-BztA-GlnR*3-Ala-NH2 |
| I-732 | Ac-PL3-Asp-Cba-B5-Asp-3COOHF-Phe-Ala-Phe-Lys*3-PyrS2-3Thi-BztA-GlnR*3-Ala-NH2 |
| I-733 | Ac-PL3-Asp-Cba-B5-Asp-3COOHF-CyLeu-Ala-Phe-Lys*3-PyrS2-3Thi-BztA-GlnR*3-Ala-NH2 |
| I-734 | Ac-PL3-Asp-Cba-B5-Asp-3COOHF-Cha-Ala-Phe-Lys*3-PyrS2-3Thi-BztA-GlnR*3-Ala-NH2 |
| I-735 | Ac-PL3-Asp-Chg-B5-Asp-3COOHF-Phe-Ala-Phe-Lys*3-PyrS2-3Thi-BztA-GlnR*3-Ala-NH2 |
| I-736 | Ac-PL3-Asp-Chg-B5-Asp-3COOHF-CyLeu-Ala-Phe-Lys*3-PyrS2-3Thi-BztA-GlnR*3-Ala-NH2 |
| I-737 | Ac-PL3-Asp-Chg-B5-Asp-3COOHF-Cha-Ala-Phe-Lys*3-PyrS2-3Thi-BztA-GlnR*3-Ala-NH2 |
| I-738 | Ac-PL3-Asp-DipA-B5-Asp-3COOHF-Phe-Ala-Phe-Lys*3-PyrS2-3Thi-BztA-GlnR*3-Ala-NH2 |
| I-739 | Ac-PL3-Asp-DipA-B5-Asp-3COOHF-Phe-Ala-Phe-Lys*3-PyrS2-3Thi-BztA-GlnR*3-Ala-NH2 |
| I-740 | Ac-PL3-Asp-DipA-B5-Asp-3COOHF-CyLeu-Ala-Phe-Lys*3-PyrS2-3Thi-BztA-GlnR*3-Ala-NH2 |
| I-741 | Ac-PL3-3COOHF-Npg-B5-Asp-Asp-Aib-Ala-Phe-Lys*3-PyrS2-3Thi-BztA-GlnR*3-Ala-NH2 |
| I-742 | Ac-PL3-Asp-Npg-B5-3COOHF-Asp-Aib-Ala-Phe-Lys*3-PyrS2-3Thi-BztA-GlnR*3-Ala-NH2 |
| I-743 | Ac-PL3-Asp-Lys**3-B5-Asp-3COOHF-GlnR**3-Ala-Phe-Lys*3-PyrS2-3Thi-BztA-GlnR*3-Ala- |
| | NH2 |
| I-744 | Ac-PL3-Asp-1MeK**3-B5-Asp-3COOHF-GlnR**3-Ala-Phe-Lys*3-PyrS2-3Thi-BztA-GlnR*3- |
| | Ala-NH2 |
| I-745 | Ac-PL3-Asp-1MeK**3-B5-Asp-3COOHF-GlnR**3-Ala-Phe-Lys*3-PyrS2-3Thi-BztA-GlnR*3- |
| | Ala-NH2 |
| I-746 | Hex-PL3-Asp-Npg-B5-Asp-3COOHF-Aib-Ala-Phe-Lys*3-PyrS2-3Thi-BztA-GlnR*3-Ala-NH2 |
| I-747 | Bua-PL3-Asp-Npg-B5-Asp-3COOHF-Aib-Ala-Phe-Lys*3-PyrS2-3Thi-BztA-GlnR*3-Ala-NH2 |
| I-748 | 2PyzCO-PL3-Asp-Npg-B5-Asp-3COOHF-Aib-Ala-Phe-Lys*3-PyrS2-3Thi-BztA-GlnR*3-Ala-NH2 |
| I-749 | 3Phc3-PL3-Asp-Npg-B5-Asp-3COOHF-Aib-Ala-Phe-Lys*3-PyrS2-3Thi-BztA-GlnR*3-Ala-NH2 |
| I-750 | MeOPr-PL3-Asp-Npg-B5-Asp-3COOHF-Aib-Ala-Phe-Lys*3-PyrS2-3Thi-BztA-GlnR*3-Ala-NH2 |
| I-751 | lithocholate-PL3-Asp-Npg-B5-Asp-3COOHF-Aib-Ala-Phe-Lys*3-PyrS2-3Thi-BztA-GlnR*3-Ala- |
| | NH2 |
| I-752 | 2FPhc-PL3-Asp-Npg-B5-Asp-3COOHF-Aib-Ala-Phe-Lys*3-PyrS2-3Thi-BztA-GlnR*3-Ala-NH2 |
| I-753 | PhC-PL3-Asp-Npg-B5-Asp-3COOHF-Aib-Ala-Phe-Lys*3-PyrS2-3Thi-BztA-GlnR*3-Ala-NH2 |
| I-754 | MeSO2-PL3-Asp-Npg-B5-Asp-3COOHF-Aib-Ala-Phe-Lys*3-PyrS2-3Thi-BztA-GlnR*3-Ala-NH2 |
| I-755 | Ts-PL3-Asp-Npg-B5-Asp-3COOHF-Aib-Ala-Phe-Lys*3-PyrS2-3Thi-BztA-GlnR*3-Ala-NH2 |
| I-756 | Ts-PL3-Asp-Npg-B5-Asp-3COOHF-Aib-Ala-Phe-Lys*3-PyrS2-3Thi-BztA-GlnR*3-Ala-NH2 |
| I-757 | Ac-PL3-Asp-Npg-B5-Asp-3COOHF-Aib-Ala-Phe-[pXyl]hCys-PyrS2-3Thi-BztA-Cys-Ala-NH2 |
| I-758 | Ac-PL3-Asp-Npg-B5-Asp-3COOHF-Aib-Ala-Phe-[pXyl]Cys-PyrS2-3Thi-BztA-hCys-Ala-NH2 |
| I-759 | Ac-PL3-Asp-Npg-B5-Asp-3COOHF-Aib-Ala-Phe-[mXyl]hCys-PyrS2-3Thi-BztA-hCys-Ala-NH2 |
| I-760 | Ac-PL3-Asp-Npg-B5-Asp-3COOHF-Aib-Ala-Phe-[pXyl]hCys-PyrS2-3Thi-BztA-hCys-Ala-NH2 |
| I-761 | Ac-PL3-Asp-Npg-B5-Asp-3COOHF-Aib-Ala-Phe-Lys*3-PyrS2-hnLeu-34ClF-GlnR*3-Ala-NH2 |
| I-762 | Ac-PL3-Asp-Npg-B5-Asp-3COOHF-Aib-Ala-Phe-Lys*3-PyrS2-hnLeu-34ClF-GlnR*3-Ala-NH2 |
| I-763 | Ac-PL3-Asp-Npg-B5-Asp-3COOHF-Aib-Ala-Phe-Lys*3-PyrS2-hnLeu-34MeF-GlnR*3-Ala-NH2 |
| I-764 | Ac-PL3-Asp-Npg-B5-Asp-3COOHF-Aib-Ala-Phe-Lys*3-PyrS2-hnLeu-34MeF-GlnR*3-Ala-NH2 |
| I-765 | Ac-PL3-Asp-Npg-B5-Asp-3COOHF-Aib-Ala-Phe-Lys*3-PyrS2-hnLeu-3BrF-GlnR*3-Ala-NH2 |
| I-766 | Ac-PL3-Asp-Npg-B5-Asp-3COOHF-Aib-Ala-Phe-Lys*3-PyrS2-hnLeu-3BrF-GlnR*3-Ala-NH2 |
| I-767 | Ac-PL3-Asp-Npg-B5-Asp-3COOHF-Aib-Ala-Phe-Lys*3-PyrS2-hnLeu-2NapA-GlnR*3-Ala-NH2 |
| I-768 | Ac-PL3-Asp-Npg-B5-Asp-3COOHF-Aib-Ala-Phe-Lys*3-PyrS2-hnLeu-2NapA-GlnR*3-Ala-NH2 |
| I-769 | Ac-PL3-Asp-Npg-B5-Asp-3COOHF-Aib-Ala-Phe-Lys*3-PyrS2-hnLeu-RbMe2NapA-GlnR*3-Ala- |
| | NH2 |
| I-770 | Ac-PL3-Asp-Npg-B5-Asp-3COOHF-Aib-Ala-Phe-Lys*3-PyrS2-hnLeu-RbMe2NapA-GlnR*3-Ala- |
| | NH2 |
| I-771 | Ac-PL3-Asp-Npg-B5-Asp-3COOHF-Aib-Ala-Phe-Lys*3-PyrS2-hnLeu-RbMeBzta-GlnR*3-Ala- |
| | NH2 |
| I-772 | Ac-PL3-Asp-Npg-B5-Asp-3COOHF-Aib-Ala-Phe-Lys*3-PyrS2-hnLeu-RbMeBzta-GlnR*3-Ala- |
| | NH2 |
| I-773 | Ac-PL3-Asp-Npg-B5-Asp-3COOHF-Aib-Ala-Phe-Lys*3-PyrS2-hnLeu-SbMeBzta-GlnR*3-Ala- |
| | NH2 |
| I-774 | Ac-PL3-Asp-Npg-B5-Asp-3COOHF-Aib-Ala-Phe-Lys*3-PyrS2-hnLeu-SbMeBzta-GlnR*3-Ala- |
| | NH2 |
| I-775 | Ac-PL3-Asp-Npg-B5-Asp-3COOHF-Aib-Ala-Phe-Lys*3-PyrS2-hnLeu-5IndA-GlnR*3-Ala-NH2 |
| I-776 | Ac-PL3-Asp-Npg-B5-Asp-3COOHF-Aib-Ala-Phe-Lys*3-PyrS2-hnLeu-5IndA-GlnR*3-Ala-NH2 |
| I-777 | Ac-PL3-Asp-Npg-B5-Asp-3COOHF-Ala-Ala-Phe-Lys*3-SPip2-2F3MeF-BztA-GlnR*3-Ala-NH2 |
| I-778 | Ac-PL3-Asp-Npg-B5-Asp-3COOHF-Ala-Ala-Phe-Lys*3-SPip3-2F3MeF-BztA-GlnR*3-Ala-NH2 |
| I-779 | Ac-PL3-Asp-Npg-B5-Asp-3COOHF-Ala-Ala-Phe-Lys*3-SPip3-2F3MeF-BztA-GlnR*3-Ala-NH2 |
| I-780 | Ac-PL3-Asp-Npg-B5-Asp-3COOHF-Ala-Ala-Phe-Lys*3-SPip2-3Thi-BztA-GlnR*3-Ala-NH2 |
| I-781 | Ac-PL3-Asp-Npg-B5-Asp-3COOHF-Ala-Ala-Phe-Lys*3-SPip3-3Thi-BztA-GlnR*3-Ala-NH2 |
| I-782 | Ac-PL3-Asp-Npg-B5-Asp-3COOHF-Aib-Ala-Phe-Lys*3-PyrS2-3Thi-BztA-GlnR*3-Ala-Ala-Ala- |
| | NH2 |
| I-783 | Ac-PL3-Asp-Npg-B5-Asp-3COOHF-Aib-Ala-Phe-Lys*3-PyrS2-3Thi-BztA-GlnR*3-Ala-Ala-Ala- |
| | Ala-Ala-NH2 |
| I-784 | Ac-PL3-Asp-Npg-B5-Asp-3COOHF-Aib-Ala-Phe-Lys*3-PyrS2-3Thi-BztA-GlnR*3-Ala-Ala-Ala- |
| | Ala-Ala-Ala-Ala-NH2 |
| I-785 | Ac-PL3-Asp-Npg-B5-Asp-3COOHF-Aib-Ala-Phe-Lys*3-PyrS2-3Thi-BztA-GlnR*3-Leu-Ser-NH2 |
| I-786 | Ac-PL3-Asp-Npg-B5-Asp-3COOHF-Aib-Ala-Phe-Lys*3-PyrS2-3Thi-BztA-hGlnR*3-NH2 |
| I-787 | Ac-PL3-Asp-Npg-B5-Asp-3COOHF-Aib-Ala-Phe-Lys*3-PyrS2-3Thi-BztA-hGlnR*3-Ala-NH2 |
| I-788 | Ac-PL3-Asp-Npg-B5-Asp-3COOHF-Aib-Ala-Phe-dDab*3-PyrS2-3Thi-BztA-GlnR*3-NH2 |
| I-789 | Ac-PL3-Asp-Npg-B5-Asp-3COOHF-Aib-Ala-Phe-dDab*3-PyrS2-3Thi-BztA-GlnR*3-Ala-Ala- |
| | Ala-NH2 |
| I-790 | Ac-PL3-Asp-Npg-B5-Asp-3COOHF-Aib-Ala-Phe-dDab*3-PyrS2-3Thi-BztA-GlnR*3-Ala-Ala- |
| | Ala-Ala-Ala-NH2 |
| I-791 | Ac-PL3-Asp-Npg-B5-Asp-3COOHF-Aib-Ala-Phe-dDab*3-PyrS2-3Thi-BztA-GlnR*3-Leu-Ser- |
| | NH2 |
| I-792 | Ac-PL3-Asp-Npg-B5-Asp-3COOHF-Aib-Ala-Phe-dOrn*3-PyrS2-3Thi-BztA-AsnR*3-NH2 |
| I-793 | Ac-PL3-Asp-Npg-B5-Asp-3COOHF-Aib-Ala-Phe-dOrn*3-PyrS2-3Thi-BztA-AsnR*3-Ala-NH2 |
| I-794 | Ac-PL3-Asp-Npg-B5-Asp-3COOHF-Aib-Ala-Phe-dOrn*3-PyrS2-3Thi-BztA-AsnR*3-Ala-NH2 |
| I-795 | Ac-PL3-Asp-Npg-B5-Asp-3COOHF-Aib-Ala-Phe-dOrn*3-PyrS2-3Thi-BztA-AsnR*3-Ala-Ala- |
| | Ala-NH2 |
| I-796 | Ac-PL3-Asp-Npg-B5-Asp-3COOHF-Aib-Ala-Phe-dOrn*3-PyrS2-3Thi-BztA-AsnR*3-Ala-Ala- |
| | Ala-NH2 |
| I-797 | Ac-PL3-Asp-Npg-B5-Asp-3COOHF-Aib-Ala-Phe-dOrn*3-PyrS2-3Thi-BztA-AsnR*3-Leu-Ser- |
| | NH2 |
| I-798 | Ac-PL3-Asp-Npg-B5-Asp-3COOHF-Aib-Ala-Phe-dOrn*3-PyrS2-3Thi-BztA-AsnR*3-Leu-Ser- |
| | NH2 |
| I-799 | Isobutyryl-PL3-Asp-Npg-B5-Asp-3COOHF-Aib-Ala-Phe-Lys*3-PyrS2-3Thi-BztA-GlnR*3-Ala- |
| | NH2 |
| I-800 | Isobutyryl-PL3-Asp-Npg-B5-Asp-3COOHF-Aib-Ala-Phe-Lys*3-PyrS2-3Thi-BztA-GlnR*3-Ala- |
| | NH2 |
| I-801 | Isovaleryl-PL3-Asp-Npg-B5-Asp-3COOHF-Aib-Ala-Phe-Lys*3-PyrS2-3Thi-BztA-GlnR*3-Ala- |
| | NH2 |
| I-802 | EtHNCO-PL3-Asp-Npg-B5-Asp-3COOHF-Aib-Ala-Phe-Lys*3-PyrS2-3Thi-BztA-GlnR*3-Ala- |
| | NH2 |
| I-803 | Ac-PL3-Asp-Npg-B5-Asp-3COOHF-Aib-Ala_D3-Phe-Lys*3-PyrS2-3Thi-BztA-GlnR*3-Ala D3- |
| | NH2 |
| I-804 | Ac-PL3-Asp-Npg-B5-Asp-3COOHF-Aib-Ala-Phe-Lys*3-PyrS2-3Thi-BztA-GlnR*3-Ala-Ala-Leu- |
| | NH2 |
| I-805 | Ac-PL3-Asp-Npg-B5-Asp-3COOHF-Aib-Ala-Phe-Lys*3-PyrS2-3Thi-BztA-GlnR*3-Ala-Ala-Leu- |
| | NH2 |
| I-806 | Ac-PL3-Asp-Npg-B5-Asp-3COOHF-Aib-Ala-Phe-Lys*3-PyrS2-3Thi-BztA-GlnR*3-Ala-Ala-Leu- |
| | Leu-NH2 |
| I-807 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-NH2 |
| I-808 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-NH2 |
| I-809 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-NH2 |
| I-810 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-Leu-NH2 |
| I-811 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-Leu-NH2 |
| I-812 | Ac-PL3-Asp-Npg-B5-Asp-3COOHF-Aib-Ala-Phe-Lys*3-PyrS2-3Thi-BztA-GlnR*3-Ala-Val-Pro- |
| | Thr-Leu-Lys-NH2 |
| I-813 | Ac-PL3-Asp-Npg-B5-Asp-3COOHF-Aib-Ala-Phe-Lys*3-PyrS2-3Thi-BztA-GlnR*3-Ala-Ala-Val- |
| | Pro-Thr-Leu-Lys-NH2 |
| I-814 | Ac-PL3-Asp-Npg-B5-Asp-3COOHF-Aib-Ala-Phe-Lys*3-PyrS2-3Thi-BztA-GlnR*3-Ala-Lys-Leu- |
| | Pro-Val-nLeu-NH2 |
| I-815 | Ac-PL3-Asp-Npg-B5-Asp-3COOHF-Aib-Ala-Phe-Lys*3-PyrS2-3Thi-BztA-GlnR*3-Ala-Ala-Lys- |
| | Leu-Pro-Val-nLeu-NH2 |
| I-816 | Ac-PL3-Asp-Npg-B5-Asp-3COOHF-Aib-Ala-Phe-Lys*3-PyrS2-3Thi-BztA-GlnR*3-Ala-Val-Pro- |
| | Ala-Leu-Arg-NH2 |
| I-817 | Ac-PL3-Asp-Npg-B5-Asp-3COOHF-Aib-Ala-Phe-Lys*3-PyrS2-3Thi-BztA-GlnR*3-Ala-Ala-Val- |
| | Pro-Ala-Leu-Arg-NH2 |
| I-818 | 5hexenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-819 | Ac-PL3-NAsp-Npg-B5-Asp-3COOHF-Aib-Ala-Phe-Lys*3-PyrS2-3Thi-BztA-GlnR*3-Ala-NH2 |
| I-820 | Ac-PL3-Asp-Npg-B5-Asp-3COOHF-Ala-Ala-Phe-Lys*3-PyrS2-2F3MeF-BztA-GlnR*3-Val-NH2 |
| I-821 | Ac-PL3-Asp-Npg-B5-Asp-3COOHF-Ala-Ala-Phe-Lys*3-PyrS2-2F3MeF-BztA-GlnR*3-Val-NH2 |
| I-822 | Ac-PL3-Asp-Npg-B5-Asp-3COOHF-Ala-Ala-Phe-Lys*3-PyrS2-2F3MeF-BztA-GlnR*3-Val-Ser- |
| | NH2 |
| I-823 | Ac-PL3-Asp-Npg-B5-Asp-3COOHF-Ala-Ala-Phe-Lys*3-PyrS2-2F3MeF-34ClF-GlnR*3-Ala-NH2 |
| I-824 | Ac-PL3-Asp-Npg-B5-Asp-3COOHF-Ala-Ala-Phe-Lys*3-PyrS2-2F3MeF-34ClF-GlnR*3-Val-NH2 |
| I-825 | Ac-PL3-Asp-Npg-B5-Asp-3COOHF-Ala-Ala-Phe-Lys*3-PyrS2-2F3MeF-34ClF-GlnR*3-Val-NH2 |
| I-826 | Ac-PL3-Asp-Npg-B5-Asp-3COOHF-Ala-Ala-Phe-Lys*3-PyrS2-2F3MeF-34ClF-GlnR*3-Val-Ser- |
| | NH2 |
| I-827 | Ac-PL3-Asp-Npg-B5-Asp-3COOHF-Ala-Ala-Phe-Lys*3-PyrS2-2F3MeF-34ClF-GlnR*3-Leu-NH2 |
| I-828 | Ac-PL3-Asp-Npg-B5-Asp-3COOHF-Ala-Ala-Phe-Lys*3-PyrS2-2F3MeF-34ClF-GlnR*3-Leu-NH2 |
| I-829 | Ac-PL3-Asp-Npg-B5-Asp-3COOHF-Ala-Ala-Phe-Lys*3-PyrS2-2F3MeF-34ClF-GlnR*3-Leu-Ser- |
| | NH2 |
| I-830 | Ac-PL3-Asp-Npg-B5-Asp-3COOHF-Ala-Ala-Phe-Lys*3-PyrS2-2F3MeF-34ClF-GlnR*3-Leu-Ser- |
| | NH2 |
| I-831 | Ac-PL3-Asp-Npg-B5-Asp-3COOHF-Ala-Ala-Phe-Lys*3-PyrS2-Phe-34ClF-GlnR*3-Ala-NH2 |
| I-832 | Ac-PL3-Asp-Npg-B5-Asp-3COOHF-Ala-Ala-Phe-Lys*3-PyrS2-Phe-34ClF-GlnR*3-Ala-NH2 |
| I-833 | Ac-PL3-Asp-Npg-B5-Asp-3COOHF-Ala-Ala-Phe-Lys*3-PyrS2-Phe-34ClF-GlnR*3-Val-NH2 |
| I-834 | Ac-PL3-Asp-Npg-B5-Asp-3COOHF-Ala-Ala-Phe-Lys*3-PyrS2-Phe-34ClF-GlnR*3-Val-NH2 |
| I-835 | Ac-PL3-Asp-Npg-B5-Asp-3COOHF-Ala-Ala-Phe-Lys*3-PyrS2-Phe-34ClF-GlnR*3-Val-Ser-NH2 |
| I-836 | Ac-PL3-Asp-Npg-B5-Asp-3COOHF-Ala-Ala-Phe-Lys*3-PyrS2-Phe-34ClF-GlnR*3-Val-Ser-NH2 |
| I-837 | Ac-PL3-Asp-Npg-B5-Asp-3COOHF-Ala-Ala-Phe-Lys*3-PyrS2-Phe-34ClF-GlnR*3-Leu-NH2 |
| I-838 | Ac-PL3-Asp-Npg-B5-Asp-3COOHF-Ala-Ala-Phe-Lys*3-PyrS2-Phe-34ClF-GlnR*3-Leu-NH2 |
| I-839 | Ac-PL3-Asp-Npg-B5-Asp-3COOHF-Ala-Ala-Phe-Lys*3-PyrS2-Phe-34ClF-GlnR*3-Leu-Ser-NH2 |
| I-840 | Ac-PL3-Asp-Npg-B5-Asp-3COOHF-Ala-Ala-Phe-Lys*3-PyrS2-Phe-34ClF-GlnR*3-Leu-Ser-NH2 |
| I-841 | Ac-PL3-Asp-Cha-B5-Asp-3COOHF-Phe-Ala-Phe-Lys*3-PyrS2-3Thi-BztA-GlnR*3-Ala-NH2 |
| I-842 | Ac-PL3-Asp-nLeu-B5-Asp-3COOHF-Phe-Ala-Phe-Lys*3-PyrS2-3Thi-BztA-GlnR*3-Ala-NH2 |
| I-843 | Ac-PL3-Asp-Ile-B5-Asp-3COOHF-Aic-Ala-Phe-Lys*3-PyrS2-3Thi-BztA-GlnR*3-Ala-NH2 |
| I-844 | Ac-PL3-Asp-Chg-B5-Asp-3COOHF-Aic-Ala-Phe-Lys*3-PyrS2-3Thi-BztA-GlnR*3-Ala-NH2 |
| I-845 | Ac-PL3-Asp-DipA-B5-Asp-3COOHF-Aic-Ala-Phe-Lys*3-PyrS2-3Thi-BztA-GlnR*3-Ala-NH2 |
| I-846 | Ac-PL3-Asp-Cha-B5-Asp-3COOHF-Aic-Ala-Phe-Lys*3-PyrS2-3Thi-BztA-GlnR*3-Ala-NH2 |
| I-847 | Ac-PL3-Asp-Cba-B5-Asp-3COOHF-Aic-Ala-Phe-Lys*3-PyrS2-3Thi-BztA-GlnR*3-Ala-NH2 |
| I-848 | Ac-PL3-Asp-nLeu-B5-Asp-3COOHF-Aic-Ala-Phe-Lys*3-PyrS2-3Thi-BztA-GlnR*3-Ala-NH2 |
| I-849 | Ac-PL3-Asp-Ile-B5-Asp-3COOHF-CyhLeu-Ala-Phe-Lys*3-PyrS2-3Thi-BztA-GlnR*3-Ala-NH2 |
| I-850 | Ac-PL3-Asp-Chg-B5-Asp-3COOHF-CyhLeu-Ala-Phe-Lys*3-PyrS2-3Thi-BztA-GlnR*3-Ala-NH2 |
| I-851 | Ac-PL3-Asp-DipA-B5-Asp-3COOHF-CyhLeu-Ala-Phe-Lys*3-PyrS2-3Thi-BztA-GlnR*3-Ala-NH2 |
| I-852 | Ac-PL3-Asp-Cha-B5-Asp-3COOHF-CyhLeu-Ala-Phe-Lys*3-PyrS2-3Thi-BztA-GlnR*3-Ala-NH2 |
| I-853 | Ac-PL3-Asp-Cha-B5-Asp-3COOHF-CyhLeu-Ala-Phe-Lys*3-PyrS2-3Thi-BztA-GlnR*3-Ala-NH2 |
| I-854 | Ac-PL3-Asp-Cba-B5-Asp-3COOHF-CyhLeu-Ala-Phe-Lys*3-PyrS2-3Thi-BztA-GlnR*3-Ala-NH2 |
| I-855 | Ac-PL3-Asp-nLeu-B5-Asp-3COOHF-CyhLeu-Ala-Phe-Lys*3-PyrS2-3Thi-BztA-GlnR*3-Ala-NH2 |
| I-856 | Ac-PL3-Asp-Ile-B5-Asp-3COOHF-Cbg-Ala-Phe-Lys*3-PyrS2-3Thi-BztA-GlnR*3-Ala-NH2 |
| I-857 | Ac-PL3-Asp-Chg-B5-Asp-3COOHF-Cbg-Ala-Phe-Lys*3-PyrS2-3Thi-BztA-GlnR*3-Ala-NH2 |
| I-858 | Ac-PL3-Asp-DipA-B5-Asp-3COOHF-Cbg-Ala-Phe-Lys*3-PyrS2-3Thi-BztA-GlnR*3-Ala-NH2 |
| I-859 | Ac-PL3-Asp-Cha-B5-Asp-3COOHF-Cbg-Ala-Phe-Lys*3-PyrS2-3Thi-BztA-GlnR*3-Ala-NH2 |
| I-860 | Ac-PL3-Asp-Cba-B5-Asp-3COOHF-Cbg-Ala-Phe-Lys*3-PyrS2-3Thi-BztA-GlnR*3-Ala-NH2 |
| I-861 | Ac-PL3-Asp-nLeu-B5-Asp-3COOHF-Cbg-Ala-Phe-Lys*3-PyrS2-3Thi-BztA-GlnR*3-Ala-NH2 |
| I-862 | Ac-PL3-Asp-Npg-B5-Asp-3COOHF-Aib-Ala-Phe-Lys*3-PyrS2-2F3MeF-34ClF-GlnR*3-NH2 |
| I-863 | Ac-PL3-Asp-Npg-B5-Asp-3COOHF-Aib-Ala-Phe-Lys*3-PyrS2-2F3MeF-34ClF-GlnR*3-NH2 |
| I-864 | Ac-PL3-Asp-Npg-B5-Asp-3COOHF-Aib-Ala-Phe-Lys*3-PyrS2-2F3MeF-34ClF-GlnR*3-Pro-NH2 |
| I-865 | Ac-PL3-Asp-Npg-B5-Asp-3COOHF-Aib-Ala-Phe-Lys*3-PyrS2-2F3MeF-34ClF-GlnR*3-Ser-NH2 |
| I-866 | Ac-PL3-Asp-Npg-B5-Asp-3COOHF-Aib-Ala-Phe-Lys*3-PyrS2-2F3MeF-34ClF-GlnR*3-Phe-NH2 |
| I-867 | Ac-PL3-Asp-Npg-B5-Asp-3COOHF-Aib-Ala-Phe-Lys*3-PyrS2-3Thi-34ClF-GlnR*3-NH2 |
| I-868 | Ac-PL3-Asp-Npg-B5-Asp-3COOHF-Aib-Ala-Phe-Lys*3-PyrS2-3Thi-34ClF-GlnR*3-Pro-NH2 |
| I-869 | Ac-PL3-Asp-Npg-B5-Asp-3COOHF-Aib-Ala-Phe-Lys*3-PyrS2-3Thi-34ClF-GlnR*3-Ser-NH2 |
| I-870 | Ac-PL3-Asp-Npg-B5-Asp-3COOHF-Aib-Ala-Phe-Lys*3-PyrS2-3Thi-34ClF-GlnR*3-Phe-NH2 |
| I-871 | Ac-PL3-Asp-Npg-B5-Asp-3COOHF-Aib-Ala-Phe-Lys*3-PyrS2-Phe-34ClF-GlnR*3-NH2 |
| I-872 | Ac-PL3-Asp-Npg-B5-Asp-3COOHF-Aib-Ala-Phe-Lys*3-PyrS2-Phe-34ClF-GlnR*3-Pro-NH2 |
| I-873 | Ac-PL3-Asp-Npg-B5-Asp-3COOHF-Aib-Ala-Phe-Lys*3-PyrS2-Phe-34ClF-GlnR*3-Ser-NH2 |
| I-874 | Ac-PL3-Asp-Npg-B5-Asp-3COOHF-Aib-Ala-Phe-Lys*3-PyrS2-Phe-34ClF-GlnR*3-Phe-NH2 |
| I-875 | Ac-PL3-Asp-Npg-B5-Asp-TfeGA-Aib-Ala-Phe-Lys*3-PyrS2-3Thi-BztA-GlnR*3-NH2 |
| I-876 | Ac-PL3-Asp-Npg-B5-Asp-TfeGA-Aib-Ala-Phe-Lys*3-PyrS2-3Thi-BztA-GlnR*3-Ala-NH2 |
| I-877 | Ac-PL3-Asp-Npg-B5-Asp-TfeGA-Aib-Ala-Phe-Lys*3-PyrS2-3Thi-BztA-GlnR*3-Leu-Ser-NH2 |
| I-878 | Ac-PL3-Asp-Npg-B5-Asp-TfeGA-Aib-Ala-Phe-Lys*3-PyrS2-Phe-BztA-GlnR*3-NH2 |
| I-879 | Ac-PL3-Asp-Npg-B5-Asp-TfeGA-Aib-Ala-Phe-Lys*3-PyrS2-Phe-BztA-GlnR*3-Ala-NH2 |
| I-880 | Ac-PL3-Asp-Npg-B5-Asp-TfeGA-Aib-Ala-Phe-Lys*3-PyrS2-Phe-BztA-GlnR*3-Leu-Ser-NH2 |
| I-881 | Ac-PL3-Asp-Npg-B5-Asp-TfeGA-Aib-Ala-Phe-Lys*3-PyrS2-3Thi-34ClF-GlnR*3-NH2 |
| I-882 | Ac-PL3-Asp-Npg-B5-Asp-TfeGA-Aib-Ala-Phe-Lys*3-PyrS2-3Thi-34ClF-GlnR*3-Ala-NH2 |
| I-883 | Ac-PL3-Asp-Npg-B5-Asp-TfeGA-Aib-Ala-Phe-Lys*3-PyrS2-3Thi-34ClF-GlnR*3-Leu-Ser-NH2 |
| I-884 | Ac-PL3-Asp-Npg-B5-Asp-TfeGA-Aib-Ala-Phe-Lys*3-PyrS2-Phe-34ClF-GlnR*3-NH2 |
| I-885 | Ac-PL3-Asp-Npg-B5-Asp-TfeGA-Aib-Ala-Phe-Lys*3-PyrS2-Phe-34ClF-GlnR*3-Ala-NH2 |
| I-886 | Ac-PL3-Asp-Npg-B5-Asp-TfeGA-Aib-Ala-Phe-Lys*3-PyrS2-Phe-34ClF-GlnR*3-Leu-Ser-NH2 |
| I-887 | Ac-PL3-Asp-Npg-B5-Asp-TfeGA-Aib-Ala-Phe-Lys*3-PyrS2-2F3MeF-BztA-GlnR*3-NH2 |
| I-888 | Ac-PL3-Asp-Npg-B5-Asp-TfeGA-Aib-Ala-Phe-Lys*3-PyrS2-2F3MeF-BztA-GlnR*3-Leu-Ser- |
| | NH2 |
| I-889 | Ac-PL3-Asp-Npg-B5-Asp-TfeGA-Aib-Ala-Phe-Lys*3-PyrS2-2F3MeF-BztA-GlnR*3-Leu-Ser- |
| | NH2 |
| I-890 | Ac-PL3-Asp-Npg-B5-Asp-TfeGA-Aib-Ala-Phe-Lys*3-PyrS2-2F3MeF-34ClF-GlnR*3-NH2 |
| I-891 | Ac-PL3-Asp-Npg-B5-Asp-TfeGA-Aib-Ala-Phe-Lys*3-PyrS2-2F3MeF-34ClF-GlnR*3-NH2 |
| I-892 | Ac-PL3-Asp-Npg-B5-Asp-TfeGA-Aib-Ala-Phe-Lys*3-PyrS2-2F3MeF-34ClF-GlnR*3-Ala-NH2 |
| I-893 | Ac-PL3-Asp-Npg-B5-Asp-TfeGA-Aib-Ala-Phe-Lys*3-PyrS2-2F3MeF-34ClF-GlnR*3-Leu-Ser- |
| | NH2 |
| I-894 | Ac-PL3-Asp-Npg-B5-Asp-TfeGA-Ala-Ala-Phe-Lys*3-PyrS2-Phe-BztA-GlnR*3-NH2 |
| I-895 | Ac-PL3-Asp-Npg-B5-Asp-TfeGA-Ala-Ala-Phe-Lys*3-PyrS2-Phe-BztA-GlnR*3-NH2 |
| I-896 | Ac-PL3-Asp-Npg-B5-Asp-TfeGA-Ala-Ala-Phe-Lys*3-PyrS2-Phe-BztA-GlnR*3-Ala-NH2 |
| I-897 | Ac-PL3-Asp-Npg-B5-Asp-TfeGA-Ala-Ala-Phe-Lys*3-PyrS2-Phe-BztA-GlnR*3-Ala-NH2 |
| I-898 | Ac-PL3-Asp-Npg-B5-Asp-TfeGA-Ala-Ala-Phe-Lys*3-PyrS2-Phe-BztA-GlnR*3-Leu-Ser-NH2 |
| I-899 | Ac-PL3-Asp-Npg-B5-Asp-TfeGA-Ala-Ala-Phe-Lys*3-PyrS2-Phe-BztA-GlnR*3-Leu-Ser-NH2 |
| I-900 | Ac-PL3-Asp-Npg-B5-Asp-TfeGA-Phe-Ala-Phe-Lys*3-PyrS2-Phe-BztA-GlnR*3-NH2 |
| I-901 | Ac-PL3-Asp-Npg-B5-Asp-TfeGA-Phe-Ala-Phe-Lys*3-PyrS2-Phe-BztA-GlnR*3-Ala-NH2 |
| I-902 | Ac-PL3-Asp-Npg-B5-Asp-TfeGA-Phe-Ala-Phe-Lys*3-PyrS2-Phe-BztA-GlnR*3-Leu-Ser-NH2 |
| I-903 | TzPyr-PL3-Asp-Npg-B5-Asp-3COOHF-Aib-Ala-Phe-Lys*3-PyrS2-3Thi-BztA-GlnR*3-Ala-NH2 |
| I-904 | 15PyraPy-PL3-Asp-Npg-B5-Asp-3COOHF-Aib-Ala-Phe-Lys*3-PyrS2-3Thi-BztA-GlnR*3-Ala- |
| | NH2 |
| I-905 | 15PyraPy-PL3-Asp-Npg-B5-Asp-3COOHF-Aib-Ala-Phe-Lys*3-PyrS2-3Thi-BztA-GlnR*3-Ala- |
| | NH2 |
| I-906 | 8IAP-PL3-Asp-Npg-B5-Asp-3COOHF-Aib-Ala-Phe-Lys*3-PyrS2-3Thi-BztA-GlnR*3-Ala-NH2 |
| I-907 | 3PydCO-PL3-Asp-Npg-B5-Asp-3COOHF-Aib-Ala-Phe-Lys*3-PyrS2-3Thi-BztA-GlnR*3-Ala- |
| | NH2 |
| I-908 | 2PyBu-PL3-Asp-Npg-B5-Asp-3COOHF-Aib-Ala-Phe-Lys*3-PyrS2-3Thi-BztA-GlnR*3-Ala-NH2 |
| I-909 | 2PymCO-PL3-Asp-Npg-B5-Asp-3COOHF-Aib-Ala-Phe-Lys*3-PyrS2-3Thi-BztA-GlnR*3-Ala- |
| | NH2 |
| I-910 | 5PymCO-PL3-Asp-Npg-B5-Asp-3COOHF-Aib-Ala-Phe-Lys*3-PyrS2-3Thi-BztA-GlnR*3-Ala- |
| | NH2 |
| I-911 | 4PymCO-PL3-Asp-Npg-B5-Asp-3COOHF-Aib-Ala-Phe-Lys*3-PyrS2-3Thi-BztA-GlnR*3-Ala- |
| | NH2 |
| I-912 | 4PymCO-PL3-Asp-Npg-B5-Asp-3COOHF-Aib-Ala-Phe-Lys*3-PyrS2-3Thi-BztA-GlnR*3-Ala- |
| | NH2 |
| I-913 | Ac-PL3-Asp-Npg-B5-Asp-3COOHF-Aib-Ala-Phe-Lys*3-PyrS2-2Thi-34ClF-GlnR*3-Ala-NH2 |
| I-914 | Ac-PL3-Asp-Npg-B5-Asp-3COOHF-Aib-Ala-Phe-Lys*3-PyrS2-2Thi-34ClF-GlnR*3-Ala-NH2 |
| I-915 | Ac-PL3-Asp-Npg-B5-Asp-3COOHF-Aib-Ala-Phe-Lys*3-PyrS2-2Thi-34ClF-GlnR*3-Leu-Ser-NH2 |
| I-916 | Ac-PL3-Asp-Npg-B5-Asp-3COOHF-Aib-Ala-Phe-Lys*3-PyrS2-2Thi-34ClF-GlnR*3-Leu-Ser-NH2 |
| I-917 | Ac-PL3-Asp-Npg-B5-Asp-3COOHF-Leu-Ala-Phe-Lys*3-PyrS2-2Thi-34ClF-GlnR*3-Ala-NH2 |
| I-918 | Ac-PL3-Asp-Npg-B5-Asp-3COOHF-Leu-Ala-Phe-Lys*3-PyrS2-2Thi-34ClF-GlnR*3-Ala-NH2 |
| I-919 | Ac-PL3-Asp-Npg-B5-Asp-3COOHF-Phe-Ala-Phe-Lys*3-PyrS2-2Thi-34ClF-GlnR*3-Ala-NH2 |
| I-920 | Ac-PL3-Asp-Npg-B5-Asp-3COOHF-Phe-Ala-Phe-Lys*3-PyrS2-2Thi-34ClF-GlnR*3-Ala-NH2 |
| I-921 | Ac-PL3-Asp-Npg-B5-Asp-3COOHF-Aib-Ala-2Thi-Lys*3-PyrS2-2Thi-34ClF-GlnR*3-Ala-NH2 |
| I-922 | Ac-PL3-Asp-Npg-B5-Asp-3COOHF-Aib-Ala-2Thi-Lys*3-PyrS2-2Thi-34ClF-GlnR*3-Ala-NH2 |
| I-923 | Ac-PL3-Asp-Npg-B5-Asp-3COOHF-Aib-Ala-Phe-Lys*3-PyrS2-2Thi-BztA-GlnR*3-NH2 |
| I-924 | Ac-PL3-Asp-Npg-B5-Asp-3COOHF-Aib-Ala-Phe-Lys*3-PyrS2-2Thi-BztA-GlnR*3-NH2 |
| I-925 | Ac-PL3-Asp-Npg-B5-Asp-3COOHF-Aib-Ala-Phe-Lys*3-PyrS2-2Thi-BztA-GlnR*3-Leu-Ser-NH2 |
| I-926 | Ac-PL3-Asp-Npg-B5-Asp-3COOHF-Leu-Ala-Phe-Lys*3-PyrS2-2Thi-BztA-GlnR*3-Ala-NH2 |
| I-927 | Ac-PL3-Asp-Npg-B5-Asp-3COOHF-Phe-Ala-Phe-Lys*3-PyrS2-2Thi-BztA-GlnR*3-Ala-NH2 |
| I-928 | Ac-PL3-Phe-Npg-B5-Asp-3COOHF-Aib-Ala-Phe-Lys*3-PyrS2-3Thi-BztA-GlnR*3-Ala-NH2 |
| I-929 | Ac-PL3-Gln-Npg-B5-Asp-3COOHF-Aib-Ala-Phe-Lys*3-PyrS2-3Thi-BztA-GlnR*3-Ala-NH2 |
| I-930 | Ac-PL3-Gln-Npg-B5-Asp-3COOHF-Aib-Ala-Phe-Lys*3-PyrS2-3Thi-BztA-GlnR*3-Ala-NH2 |
| I-931 | Ac-PL3-3Thi-Npg-B5-Asp-3COOHF-Aib-Ala-Phe-Lys*3-PyrS2-3Thi-BztA-GlnR*3-Ala-NH2 |
| I-932 | 4pentenyl-MePro-Asp-B5-Ala-Asp-3COOHF-Ala-Ala-Phe-PyrS2-Lys*3-3Thi-BztA-Ala-GlnR*3- |
| | NH2 |
| I-933 | 4pentenyl-ThioPro-Asp-B5-Ala-Asp-3COOHF-Ala-Ala-Phe-PyrS2-Lys*3-3Thi-BztA-Ala-GlnR*3- |
| | NH2 |
| I-934 | 4pentenyl-ThioPro-Asp-B5-Ala-Asp-3COOHF-Ala-Ala-Phe-PyrS2-Lys*3-3Thi-BztA-Ala-GlnR*3- |
| | NH2 |
| I-935 | Ac-PL3-NGlu-Npg-B5-Asp-3COOHF-Aib-Ala-Phe-Lys*3-PyrS2-3Thi-BztA-GlnR*3-Ala-NH2 |
| I-936 | Ac-PL3-Asp-Npg-B5-Asp-3COOHF-Aib-Ala-Phe-Lys*3-PyrS2-HexG-BztA-GlnR*3-Ala-NH2 |
| I-937 | Ac-PL3-Asp-Npg-B5-Asp-3COOHF-Aib-Ala-Phe-Lys*3-PyrS2-HexG-BztA-GlnR*3-Ala-NH2 |
| I-938 | Ac-PL3-Asp-Npg-B5-Asp-3COOHF-Aib-Ala-Phe-Lys*3-PyrS2-HepG-BztA-GlnR*3-Ala-NH2 |
| I-939 | Ac-PL3-Asp-Npg-B5-Asp-3COOHF-Aib-Ala-Phe-Lys*3-PyrS2-HepG-BztA-GlnR*3-Ala-NH2 |
| I-940 | Ac-PL3-Asp-Npg-B5-Asp-3COOHF-Aib-Ala-Phe-Lys*3-PyrS2-BztA-BztA-GlnR*3-Ala-NH2 |
| I-941 | Ac-PL3-Asp-Npg-B5-Asp-3COOHF-Aib-Ala-Phe-Lys*3-PyrS2-BztA-BztA-GlnR*3-Ala-NH2 |
| I-942 | Ac-PL3-Asp-Npg-B5-Asp-3COOHF-Aib-Ala-Phe-Lys*3-PyrS2-HexG-34ClF-GlnR*3-Ala-NH2 |
| I-943 | Ac-PL3-Asp-Npg-B5-Asp-3COOHF-Aib-Ala-Phe-Lys*3-PyrS2-HepG-34ClF-GlnR*3-Ala-NH2 |
| I-944 | Ac-PL3-Asp-Npg-B5-Asp-3COOHF-Aib-Ala-Phe-Lys*3-PyrS2-HepG-34ClF-GlnR*3-Ala-NH2 |
| I-945 | Ac-PL3-Asp-Npg-B5-Asp-3COOHF-Aib-Ala-Phe-Lys*3-PyrS2-BztA-34ClF-GlnR*3-Ala-NH2 |
| I-946 | Ac-PL3-Asp-Npg-B5-Asp-3COOHF-Aib-Ala-Phe-Lys*3-Az3-2F3MeF-BztA-GlnR*3-Ala-NH2 |
| I-947 | Ac-PL3-Asp-Npg-B5-Asp-3COOHF-Aib-Ala-Phe-Lys*3-Az2-3Thi-BztA-GlnR*3-Ala-NH2 |
| I-948 | Ac-PL3-Asp-Npg-B5-Asp-3COOHF-Aib-Ala-Phe-Lys*3-Az3-3Thi-BztA-GlnR*3-Ala-NH2 |
| I-949 | Ac-PL3-Asp-Npg-B5-Asp-3COOHF-Aib-Ala-Phe-Lys*3-PyrS2-2PhF-BztA-GlnR*3-Ala-NH2 |
| I-950 | Ac-PL3-Asp-Npg-B5-Asp-3COOHF-Aib-Ala-Phe-Lys*3-PyrS2-2PhF-34ClF-GlnR*3-Ala-NH2 |
| I-951 | Ac-PL3-Asp-Npg-B5-Asp-3COOHF-Aib-Ala-Phe-Lys*3-PyrS2-2PhF-34ClF-GlnR*3-Ala-NH2 |
| I-952 | Ac-PL3-Asp-Npg-B5-Asp-3COOHF-Aib-Ala-Phe-Lys*3-PyrS2-2PhF-BztA-GlnR*3-Leu-Ser-NH2 |
| I-953 | Ac-PL3-Asp-Npg-B5-Asp-3COOHF-Aib-Ala-Phe-Lys*3-PyrS2-2PhF-BztA-GlnR*3-Leu-Ser-NH2 |
| I-954 | Ac-PL3-Asp-Npg-B5-Asp-3COOHF-Aib-Ala-Phe-Lys*3-PyrS2-2PhF-34ClF-GlnR*3-Leu-Ser- |
| | NH2 |
| I-955 | Ac-PL3-Asp-Npg-B5-Asp-3COOHF-Aib-Ala-Phe-Lys*3-PyrS2-hnLeu-BztA-GlnR*3-Leu-Ser- |
| | NH2 |
| I-956 | Ac-PL3-Asp-Npg-B5-Asp-3COOHF-Aib-Ala-Phe-Lys*3-PyrS2-hnLeu-34ClF-GlnR*3-Leu-Ser- |
| | NH2 |
| I-957 | Ac-PL3-isoDAsp-Npg-B5-Asp-3COOHF-Aib-Ala-Phe-Lys*3-PyrS2-3Thi-BztA-GlnR*3-Ala-NH2 |
| I-958 | Ac-PL3-isoGlu-Npg-B5-Asp-3COOHF-Aib-Ala-Phe-Lys*3-PyrS2-3Thi-BztA-GlnR*3-Ala-NH2 |
| I-959 | Ac-PL3-isoGlu-Npg-B5-Asp-3COOHF-Aib-Ala-Phe-Lys*3-PyrS2-3Thi-BztA-GlnR*3-Ala-NH2 |
| I-960 | Ac-PL3-isoDGlu-Npg-B5-Asp-3COOHF-Aib-Ala-Phe-Lys*3-PyrS2-3Thi-BztA-GlnR*3-Ala-NH2 |
| I-961 | Ac-PL3-RbGlu-Npg-B5-Asp-3COOHF-Aib-Ala-Phe-Lys*3-PyrS2-3Thi-BztA-GlnR*3-Ala-NH2 |
| I-962 | Ac-PL3-SbGlu-Npg-B5-Asp-3COOHF-Aib-Ala-Phe-Lys*3-PyrS2-3Thi-BztA-GlnR*3-Ala-NH2 |
| I-963 | Ac-PL3-isoDAsp-Npg-B5-Asp-3COOHF-Aib-Ala-Phe-Lys*3-PyrS2-3Thi-BztA-GlnR*3-Ala-NH2 |
| I-964 | Ac-PL3-isoGlu-Npg-B5-Asp-3COOHF-Aib-Ala-Phe-Lys*3-PyrS2-3Thi-BztA-GlnR*3-Ala-NH2 |
| I-965 | Ac-PL3-RbGlu-Npg-B5-Asp-3COOHF-Aib-Ala-Phe-Lys*3-PyrS2-3Thi-BztA-GlnR*3-Ala-NH2 |
| I-966 | Ac-PL3-SbGlu-Npg-B5-Asp-3COOHF-Aib-Ala-Phe-Lys*3-PyrS2-3Thi-BztA-GlnR*3-Ala-NH2 |
| I-967 | Ac-PL3-Glu-Npg-B5-Asp-3COOHF-Aib-Ala-Phe-Lys*3-PyrS2-3Thi-BztA-GlnR*3-Ala-NH2 |
| I-968 | Ac-PL3-Gln-Npg-B5-Asp-3COOHF-Aib-Ala-Phe-Lys*3-PyrS2-3Thi-BztA-GlnR*3-Ala-NH2 |
| I-969 | Ac-PL3-Asp-Ile-B5-Asp-3COOHF-Ala-Ala-Phe-Lys*3-PyrS2-3Thi-BztA-GlnR*3-Ala-NH2 |
| I-970 | Ac-PL3-Asp-Ile-B5-Asp-3COOHF-Ala-Ala-Phe-Lys*3-PyrS2-3Thi-BztA-GlnR*3-Ala-NH2 |
| I-971 | Ac-PL3-Asp-Ile-B5-Asp-3COOHF-Val-Ala-Phe-Lys*3-PyrS2-3Thi-BztA-GlnR*3-Ala-NH2 |
| I-972 | Ac-PL3-Asp-Ile-B5-Asp-3COOHF-Leu-Ala-Phe-Lys*3-PyrS2-3Thi-BztA-GlnR*3-Ala-NH2 |
| I-973 | Ac-PL3-Asp-Ile-B5-Asp-3COOHF-nLeu-Ala-Phe-Lys*3-PyrS2-3Thi-BztA-GlnR*3-Ala-NH2 |
| I-974 | Ac-PL3-Asp-Ile-B5-Asp-3COOHF-nLeu-Ala-Phe-Lys*3-PyrS2-3Thi-BztA-GlnR*3-Ala-NH2 |
| I-975 | Ac-PL3-Asp-Ile-B5-Asp-3COOHF-Dpg-Ala-Phe-Lys*3-PyrS2-3Thi-BztA-GlnR*3-Ala-NH2 |
| I-976 | Ac-PL3-Asp-Chg-B5-Asp-3COOHF-Ala-Ala-Phe-Lys*3-PyrS2-3Thi-BztA-GlnR*3-Ala-NH2 |
| I-977 | Ac-PL3-Asp-Chg-B5-Asp-3COOHF-Ala-Ala-Phe-Lys*3-PyrS2-3Thi-BztA-GlnR*3-Ala-NH2 |
| I-978 | Ac-PL3-Asp-Chg-B5-Asp-3COOHF-Val-Ala-Phe-Lys*3-PyrS2-3Thi-BztA-GlnR*3-Ala-NH2 |
| I-979 | Ac-PL3-Asp-Chg-B5-Asp-3COOHF-nLeu-Ala-Phe-Lys*3-PyrS2-3Thi-BztA-GlnR*3-Ala-NH2 |
| I-980 | Ac-PL3-Asp-Chg-B5-Asp-3COOHF-Dpg-Ala-Phe-Lys*3-PyrS2-3Thi-BztA-GlnR*3-Ala-NH2 |
| I-981 | Ac-PL3-Asp-Chg-B5-Asp-3COOHF-Dpg-Ala-Phe-Lys*3-PyrS2-3Thi-BztA-GlnR*3-Ala-NH2 |
| I-982 | Ac-PL3-Asp-DipA-B5-Asp-3COOHF-Ala-Ala-Phe-Lys*3-PyrS2-3Thi-BztA-GlnR*3-Ala-NH2 |
| I-983 | Ac-PL3-Asp-DipA-B5-Asp-3COOHF-Ala-Ala-Phe-Lys*3-PyrS2-3Thi-BztA-GlnR*3-Ala-NH2 |
| I-984 | Ac-PL3-Asp-DipA-B5-Asp-3COOHF-Val-Ala-Phe-Lys*3-PyrS2-3Thi-BztA-GlnR*3-Ala-NH2 |
| I-985 | Ac-PL3-Asp-DipA-B5-Asp-3COOHF-Leu-Ala-Phe-Lys*3-PyrS2-3Thi-BztA-GlnR*3-Ala-NH2 |
| I-986 | Ac-PL3-Asp-DipA-B5-Asp-3COOHF-nLeu-Ala-Phe-Lys*3-PyrS2-3Thi-BztA-GlnR*3-Ala-NH2 |
| I-987 | Ac-PL3-Asp-Cba-B5-Asp-3COOHF-Ala-Ala-Phe-Lys*3-PyrS2-3Thi-BztA-GlnR*3-Ala-NH2 |
| I-988 | Ac-PL3-Asp-Cba-B5-Asp-3COOHF-Val-Ala-Phe-Lys*3-PyrS2-3Thi-BztA-GlnR*3-Ala-NH2 |
| I-989 | Ac-PL3-Asp-Cba-B5-Asp-3COOHF-Leu-Ala-Phe-Lys*3-PyrS2-3Thi-BztA-GlnR*3-Ala-NH2 |
| I-990 | Ac-PL3-Asp-Cba-B5-Asp-3COOHF-nLeu-Ala-Phe-Lys*3-PyrS2-3Thi-BztA-GlnR*3-Ala-NH2 |
| I-991 | Ac-PL3-Asp-Cba-B5-Asp-3COOHF-Dpg-Ala-Phe-Lys*3-PyrS2-3Thi-BztA-GlnR*3-Ala-NH2 |
| I-992 | 5hexenyl-MePro-Asp-B5-Ala-Asp-3COOHF-Ala-Ala-Phe-PyrS2-Lys*3-3Thi-BztA-Ala-GlnR*3- |
| | NH2 |
| I-993 | 4pentenyl-MePro-Asp-B5-Ala-Asp-3COOHF-Ala-Ala-Phe-PyrS2-Lys*3-3Thi-BztA-Ala-GlnR*3- |
| | NH2 |
| I-994 | Ac-PL3-Asp-Chg-B5-Asp-3COOHF-Gly-Ala-Phe-Lys*3-PyrS2-3Thi-BztA-GlnR*3-Ala-NH2 |
| I-995 | Ac-PL3-Asp-Chg-B5-Asp-3COOHF-Gly-Ala-Phe-Lys*3-PyrS2-3Thi-BztA-GlnR*3-Ala-NH2 |
| I-996 | Ac-PL3-Asp-Chg-B5-Asp-3COOHF-dAla-Ala-Phe-Lys*3-PyrS2-3Thi-BztA-GlnR*3-Ala-NH2 |
| I-997 | Ac-PL3-Asp-Chg-B5-Asp-3COOHF-Aib-Thr-Phe-Lys*3-PyrS2-3Thi-BztA-GlnR*3-Ala-NH2 |
| I-998 | Ac-PL3-Asp-Chg-B5-Asp-3COOHF-Aib-aThr-Phe-Lys*3-PyrS2-3Thi-BztA-GlnR*3-Ala-NH2 |
| I-999 | Ac-PL3-Asp-Chg-B5-Asp-3COOHF-Aib-Gly-Phe-Lys*3-PyrS2-3Thi-BztA-GlnR*3-Ala-NH2 |
| I-1000 | Ac-PL3-Asp-DipA-B5-Asp-3COOHF-Gly-Ala-Phe-Lys*3-PyrS2-3Thi-BztA-GlnR*3-Ala-NH2 |
| I-1001 | Ac-PL3-Asp-DipA-B5-Asp-3COOHF-Gly-Ala-Phe-Lys*3-PyrS2-3Thi-BztA-GlnR*3-Ala-NH2 |
| I-1002 | Ac-PL3-Asp-DipA-B5-Asp-3COOHF-dAla-Ala-Phe-Lys*3-PyrS2-3Thi-BztA-GlnR*3-Ala-NH2 |
| I-1003 | Ac-PL3-Asp-DipA-B5-Asp-3COOHF-dAla-Ala-Phe-Lys*3-PyrS2-3Thi-BztA-GlnR*3-Ala-NH2 |
| I-1004 | Ac-PL3-Asp-DipA-B5-Asp-3COOHF-Aib-Ser-Phe-Lys*3-PyrS2-3Thi-BztA-GlnR*3-Ala-NH2 |
| I-1005 | Ac-PL3-Asp-DipA-B5-Asp-3COOHF-Aib-Thr-Phe-Lys*3-PyrS2-3Thi-BztA-GlnR*3-Ala-NH2 |
| I-1006 | Ac-PL3-Asp-DipA-B5-Asp-3COOHF-Aib-aThr-Phe-Lys*3-PyrS2-3Thi-BztA-GlnR*3-Ala-NH2 |
| I-1007 | Ac-PL3-Asp-DipA-B5-Asp-3COOHF-Aib-aThr-Phe-Lys*3-PyrS2-3Thi-BztA-GlnR*3-Ala-NH2 |
| I-1008 | Ac-PL3-Asp-DipA-B5-Asp-3COOHF-Aib-Gly-Phe-Lys*3-PyrS2-3Thi-BztA-GlnR*3-Ala-NH2 |
| I-1009 | Ac-PL3-Asp-DipA-B5-Asp-3COOHF-Aib-Gly-Phe-Lys*3-PyrS2-3Thi-BztA-GlnR*3-Ala-NH2 |
| I-1010 | Ac-PL3-Asp-Ile-B5-Asp-3COOHF-Aib-Ala-Phe-Lys*3-PyrS2-3Thi-34ClF-GlnR*3-Ala-NH2 |
| I-1011 | Ac-PL3-Asp-Npg-B5-Asp-3COOHF-CyLeu-Ser-Phe-Lys*3-PyrS2-3Thi-BztA-GlnR*3-Ala-NH2 |
| I-1012 | Ac-PL3-Asp-Npg-B5-Asp-3COOHF-Phe-Ser-Phe-Lys*3-PyrS2-3Thi-BztA-GlnR*3-Ala-NH2 |
| I-1013 | Ac-PL3-Asp-Npg-B5-Asp-3COOHF-Val-Ser-Phe-Lys*3-PyrS2-3Thi-BztA-GlnR*3-Ala-NH2 |
| I-1014 | Ac-PL3-Asp-Npg-B5-Asp-3COOHF-CyLeu-Gly-Phe-Lys*3-PyrS2-3Thi-BztA-GlnR*3-Ala-NH2 |
| I-1015 | Ac-PL3-Asp-Npg-B5-Asp-3COOHF-Phe-Gly-Phe-Lys*3-PyrS2-3Thi-BztA-GlnR*3-Ala-NH2 |
| I-1016 | Ac-PL3-Asp-Npg-B5-Asp-3COOHF-Val-Gly-Phe-Lys*3-PyrS2-3Thi-BztA-GlnR*3-Ala-NH2 |
| I-1017 | Ac-PL3-Asp-Npg-B5-Asp-3COOHF-CyLeu-nLeu-Phe-Lys*3-PyrS2-3Thi-BztA-GlnR*3-Ala-NH2 |
| I-1018 | Ac-PL3-Asp-Npg-B5-Asp-3COOHF-Phe-nLeu-Phe-Lys*3-PyrS2-3Thi-BztA-GlnR*3-Ala-NH2 |
| I-1019 | Ac-PL3-Asp-Npg-B5-Asp-3COOHF-Val-nLeu-Phe-Lys*3-PyrS2-3Thi-BztA-GlnR*3-Ala-NH2 |
| I-1020 | Ac-PL3-Asp-Npg-B5-Asp-3COOHF-CyLeu-aThr-Phe-Lys*3-PyrS2-3Thi-BztA-GlnR*3-Ala-NH2 |
| I-1021 | Ac-PL3-Asp-Npg-B5-Asp-3COOHF-Val-aThr-Phe-Lys*3-PyrS2-3Thi-BztA-GlnR*3-Ala-NH2 |
| I-1022 | 5hexenyl-MePro-Asp-S3-R5-Asp-3COOHF-Aib-Ala-Phe-Lys*3-PyrS2-3Thi-BztA-GlnR*3-Ala- |
| | NH2 |
| I-1023 | Ac-PL3-Asp-Npg-B5-Asp-3COOHF-Aib-Ala-Phe-Lys*3-PyrS1-3Thi-BztA-GlnR*3-Ala-NH2 |
| I-1024 | Ac-PL3-Glu-Npg-B5-Glu-3COOHF-Aib-Ala-Phe-Lys*3-PyrS2-3Thi-34ClF-GlnR*3-NH2 |
| I-1025 | 2PyBu-PL3-Glu-Npg-B5-Glu-3COOHF-Aib-Ala-Phe-Lys*3-PyrS2-3Thi-BztA-GlnR*3-Ala-NH2 |
| I-1026 | Ac-PL3-Asp-Npg-B5-Asp-3COOHF-[Ac]Acp-Ala-Phe-Lys*3-PyrS2-3Thi-BztA-GlnR*3-Ala-NH2 |
| I-1027 | Ac-PL3-Asp-Npg-B5-Asp-3COOHF-[Ac]Acp-Ala-Phe-Lys*3-PyrS2-3Thi-BztA-GlnR*3-Ala-NH2 |
| I-1028 | Ac-PL3-Asp-Npg-B5-Asp-3COOHF-[Phc]Acp-Ala-Phe-Lys*3-PyrS2-3Thi-BztA-GlnR*3-Ala-NH2 |
| I-1029 | Ac-PL3-Asp-Npg-B5-Asp-3COOHF-[Phc]Acp-Ala-Phe-Lys*3-PyrS2-3Thi-BztA-GlnR*3-Ala-NH2 |
| I-1030 | Ac-PL3-Asp-Npg-B5-Asp-3COOHF-[isovaleryl]Acp-Ala-Phe-Lys*3-PyrS2-3Thi-BztA-GlnR*3- |
| | Ala-NH2 |
| I-1031 | Ac-PL3-Asp-Npg-B5-Asp-3COOHF-[Ac]PyrSa-Ala-Phe-Lys*3-PyrS2-3Thi-BztA-GlnR*3-Ala- |
| | NH2 |
| I-1032 | Ac-PL3-Asp-Npg-B5-Asp-3COOHF-[Phc]PyrSa-Ala-Phe-Lys*3-PyrS2-3Thi-BztA-GlnR*3-Ala- |
| | NH2 |
| I-1033 | Ac-PL3-Asp-Npg-B5-Asp-3COOHF-[isovaleryl]PyrSa-Ala-Phe-Lys*3-PyrS2-3Thi-BztA-GlnR*3- |
| | Ala-NH2 |
| I-1034 | Ac-PL3-Asp-Npg-B5-Asp-3COOHF-[Ac]PyrRa-Ala-Phe-Lys*3-PyrS2-3Thi-BztA-GlnR*3-Ala- |
| | NH2 |
| I-1035 | Ac-PL3-Asp-Npg-B5-Asp-3COOHF-[Phc]PyrRa-Ala-Phe-Lys*3-PyrS2-3Thi-BztA-GlnR*3-Ala- |
| | NH2 |
| I-1036 | Ac-PL3-Asp-Npg-B5-Asp-3COOHF-[isovaleryl]PyrRa-Ala-Phe-Lys*3-PyrS2-3Thi-BztA-GlnR*3- |
| | Ala-NH2 |
| I-1037 | Ac-PL3-Asp-Chg-B5-Asp-3COOHF-Aib-Ala-Phe-Lys*3-PyrS2-3Thi-34ClF-GlnR*3-NH2 |
| I-1038 | Ac-PL3-Asp-Cha-B5-Asp-3COOHF-Aib-Ala-Phe-Lys*3-PyrS2-3Thi-34ClF-GlnR*3-NH2 |
| I-1039 | Ac-PL3-Asp-Cha-B5-Asp-3COOHF-Aib-Ala-Phe-Lys*3-PyrS2-3Thi-34ClF-GlnR*3-NH2 |
| I-1040 | Ac-PL3-Asp-Ile-B5-Asp-3COOHF-CyLeu-Ala-Phe-Lys*3-PyrS2-3Thi-34ClF-GlnR*3-NH2 |
| I-1041 | Ac-PL3-Asp-Ile-B5-Asp-3COOHF-CyLeu-Ala-Phe-Lys*3-PyrS2-3Thi-34ClF-GlnR*3-NH2 |
| I-1042 | Ac-PL3-Asp-Npg-B5-Asp-3COOHF-CyLeu-Ala-Phe-Lys*3-PyrS2-3Thi-34ClF-GlnR*3-NH2 |
| I-1043 | Ac-PL3-Asp-Npg-B5-Asp-3COOHF-CyLeu-Ala-Phe-Lys*3-PyrS2-3Thi-34ClF-GlnR*3-NH2 |
| I-1044 | Ac-PL3-Asp-Chg-B5-Asp-3COOHF-CyLeu-Ala-Phe-Lys*3-PyrS2-3Thi-34ClF-GlnR*3-NH2 |
| I-1045 | Ac-PL3-Asp-Chg-B5-Asp-3COOHF-CyLeu-Ala-Phe-Lys*3-PyrS2-3Thi-34ClF-GlnR*3-NH2 |
| I-1046 | Ac-PL3-Asp-Cha-B5-Asp-3COOHF-CyLeu-Ala-Phe-Lys*3-PyrS2-3Thi-34ClF-GlnR*3-NH2 |
| I-1047 | Ac-PL3-Asp-Cha-B5-Asp-3COOHF-CyLeu-Ala-Phe-Lys*3-PyrS2-3Thi-34ClF-GlnR*3-NH2 |
| I-1048 | Ac-PL3-Asp-Chg-B5-Asp-3COOHF-Aib-Ala-Phe-Lys*3-PyrS2-Phe-34ClF-GlnR*3-NH2 |
| I-1049 | Ac-PL3-Asp-Chg-B5-Asp-3COOHF-Aib-Ala-Phe-Lys*3-PyrS2-Phe-34ClF-GlnR*3-NH2 |
| I-1050 | Ac-PL3-Asp-Cha-B5-Asp-3COOHF-Aib-Ala-Phe-Lys*3-PyrS2-Phe-34ClF-GlnR*3-NH2 |
| I-1051 | Ac-PL3-Asp-Cha-B5-Asp-3COOHF-Aib-Ala-Phe-Lys*3-PyrS2-Phe-34ClF-GlnR*3-NH2 |
| I-1052 | Ac-PL3-Asp-Ile-B5-Asp-3COOHF-CyLeu-Ala-Phe-Lys*3-PyrS2-Phe-34ClF-GlnR*3-NH2 |
| I-1053 | Ac-PL3-Asp-Ile-B5-Asp-3COOHF-CyLeu-Ala-Phe-Lys*3-PyrS2-Phe-34ClF-GlnR*3-NH2 |
| I-1054 | Ac-PL3-Asp-Npg-B5-Asp-3COOHF-CyLeu-Ala-Phe-Lys*3-PyrS2-Phe-34ClF-GlnR*3-NH2 |
| I-1055 | Ac-PL3-Asp-Npg-B5-Asp-3COOHF-CyLeu-Ala-Phe-Lys*3-PyrS2-Phe-34ClF-GlnR*3-NH2 |
| I-1056 | Ac-PL3-Asp-Chg-B5-Asp-3COOHF-CyLeu-Ala-Phe-Lys*3-PyrS2-Phe-34ClF-GlnR*3-NH2 |
| I-1057 | Ac-PL3-Asp-Chg-B5-Asp-3COOHF-CyLeu-Ala-Phe-Lys*3-PyrS2-Phe-34ClF-GlnR*3-NH2 |
| I-1058 | Ac-PL3-Asp-Cha-B5-Asp-3COOHF-CyLeu-Ala-Phe-Lys*3-PyrS2-Phe-34ClF-GlnR*3-NH2 |
| I-1059 | Ac-PL3-Asp-Cha-B5-Asp-3COOHF-CyLeu-Ala-Phe-Lys*3-PyrS2-Phe-34ClF-GlnR*3-NH2 |
| I-1060 | Ac-PL3-Asp-Npg-B5-Asp-SbMeAsp-Aib-Ala-Phe-Lys*3-PyrS2-Phe-34ClF-GlnR*3-NH2 |
| I-1061 | Ac-PL3-Asp-Npg-B5-Asp-bMe2Asp-Aib-Ala-Phe-Lys*3-PyrS2-Phe-34ClF-GlnR*3-NH2 |
| I-1062 | 1Imidac-PL3-Asp-Npg-B5-Asp-3COOHF-Aib-Ala-Phe-Lys*3-PyrS2-3Thi-BztA-GlnR*3-Ala-NH2 |
| I-1063 | 2F2PyAc-PL3-Asp-Npg-B5-Asp-3COOHF-Aib-Ala-Phe-Lys*3-PyrS2-3Thi-BztA-GlnR*3-Ala- |
| | NH2 |
| I-1064 | 2IAPAc-PL3-Asp-Npg-B5-Asp-3COOHF-Aib-Ala-Phe-Lys*3-PyrS2-3Thi-BztA-GlnR*3-Ala-NH2 |
| I-1065 | 124TriPr-PL3-Asp-Npg-B5-Asp-3COOHF-Aib-Ala-Phe-Lys*3-PyrS2-3Thi-BztA-GlnR*3-Ala- |
| | NH2 |
| I-1066 | 6QuiAc-PL3-Asp-Npg-B5-Asp-3COOHF-Aib-Ala-Phe-Lys*3-PyrS2-3Thi-BztA-GlnR*3-Ala-NH2 |
| I-1067 | 3Py Ac-PL3-Asp-Npg-B5-Asp-3COOHF-Aib-Ala-Phe-Lys*3-PyrS2-3Thi-BztA-GlnR*3-Ala-NH2 |
| I-1068 | 123TriAc-PL3-Asp-Npg-B5-Asp-3COOHF-Aib-Ala-Phe-Lys*3-PyrS2-3Thi-BztA-GlnR*3-Ala- |
| | NH2 |
| I-1069 | 1Pyrazole Ac-PL3-Asp-Npg-B5-Asp-3COOHF-Aib-Ala-Phe-Lys*3-PyrS2-3Thi-BztA-GlnR*3-Ala- |
| | NH2 |
| I-1070 | 4PyPrpc-PL3-Asp-Npg-B5-Asp-3COOHF-Aib-Ala-Phe-Lys*3-PyrS2-3Thi-BztA-GlnR*3-Ala-NH2 |
| I-1071 | 4PyPrpc-PL3-Asp-Npg-B5-Asp-3COOHF-Aib-Ala-Phe-Lys*3-PyrS2-3Thi-BztA-GlnR*3-Ala-NH2 |
| I-1072 | 3PyPrpc-PL3-Asp-Npg-B5-Asp-3COOHF-Aib-Ala-Phe-Lys*3-PyrS2-3Thi-BztA-GlnR*3-Ala-NH2 |
| I-1073 | 5PymAc-PL3-Asp-Npg-B5-Asp-3COOHF-Aib-Ala-Phe-Lys*3-PyrS2-3Thi-BztA-GlnR*3-Ala-NH2 |
| I-1074 | 1PydoneAc-PL3-Asp-Npg-B5-Asp-3COOHF-Aib-Ala-Phe-Lys*3-PyrS2-3Thi-BztA-GlnR*3-Ala- |
| | NH2 |
| I-1075 | 124TriAc-PL3-Asp-Npg-B5-Asp-3COOHF-Aib-Ala-Phe-Lys*3-PyrS2-3Thi-BztA-GlnR*3-Ala- |
| | NH2 |
| I-1076 | 3IAPAc-PL3-Asp-Npg-B5-Asp-3COOHF-Aib-Ala-Phe-Lys*3-PyrS2-3Thi-BztA-GlnR*3-Ala-NH2 |
| I-1077 | 3IAPAc-PL3-Asp-Npg-B5-Asp-3COOHF-Aib-Ala-Phe-Lys*3-PyrS2-3Thi-BztA-GlnR*3-Ala-NH2 |
| I-1078 | Me2NAc-PL3-Asp-Npg-B5-Asp-3COOHF-Aib-Ala-Phe-Lys*3-PyrS2-3Thi-BztA-GlnR*3-Ala- |
| | NH2 |
| I-1079 | 4MePipzPrpC-PL3-Asp-Npg-B5-Asp-3COOHF-Aib-Ala-Phe-Lys*3-PyrS2-3Thi-BztA-GlnR*3- |
| | Ala-NH2 |
| I-1080 | 4MePipzPrpC-PL3-Asp-Npg-B5-Asp-3COOHF-Aib-Ala-Phe-Lys*3-PyrS2-3Thi-BztA-GlnR*3- |
| | Ala-NH2 |
| I-1081 | MePipAc-PL3-Asp-Npg-B5-Asp-3COOHF-Aib-Ala-Phe-Lys*3-PyrS2-3Thi-BztA-GlnR*3-Ala- |
| | NH2 |
| I-1082 | MePipAc-PL3-Asp-Npg-B5-Asp-3COOHF-Aib-Ala-Phe-Lys*3-PyrS2-3Thi-BztA-GlnR*3-Ala- |
| | NH2 |
| I-1083 | MeImid4SO2-PL3-Asp-Npg-B5-Asp-3COOHF-Aib-Ala-Phe-Lys*3-PyrS2-3Thi-BztA-GlnR*3- |
| | Ala-NH2 |
| I-1084 | MeImid4SO2-PL3-Asp-Npg-B5-Asp-3COOHF-Aib-Ala-Phe-Lys*3-PyrS2-3Thi-BztA-GlnR*3- |
| | Ala-NH2 |
| I-1085 | 8QuiSO2-PL3-Asp-Npg-B5-Asp-3COOHF-Aib-Ala-Phe-Lys*3-PyrS2-3Thi-BztA-GlnR*3-Ala- |
| | NH2 |
| I-1086 | Ac-PL3-Asp-Npg-B5-Asp-3COOHF-Aib-Ala-Phe-TriAzLys*3-PyrS2-3Thi-34ClF-sAla*3-NH2 |
| I-1087 | Ac-PL3-Asp-Npg-B5-Asp-3COOHF-Aib-Ala-Phe-TriAzLys*3-PyrS2-3Thi-34ClF-sAla*3-NH2 |
| I-1088 | Ac-PL3-Asp-Npg-B5-Asp-3COOHF-Aib-Ala-Phe-TriAzLys*3-PyrS2-3Thi-34ClF-sAla*3-Ala- |
| | NH2 |
| I-1089 | Ac-PL3-Asp-Ile-B5-Asp-3COOHF-Aib-Ala-Phe-TriAzLys*3-PyrS2-3Thi-34ClF-sAla*3-NH2 |
| I-1090 | Ac-PL3-Asp-Ile-B5-Asp-3COOHF-Phe-Ala-Phe-TriAzLys*3-PyrS2-3Thi-34ClF-sAla*3-NH2 |
| I-1091 | Ac-PL3-Asp-Ile-B5-Asp-3COOHF-Phe-Ala-Phe-TriAzLys*3-PyrS2-3Thi-34ClF-sAla*3-Ala-NH2 |
| I-1092 | Ac-PL3-Asp-Npg-B5-Asp-3COOHF-Aib-Ala-Phe-TriAzLys*3-PyrS2-Phe-34ClF-sAla*3-NH2 |
| I-1093 | Ac-PL3-Asp-Npg-B5-Asp-3COOHF-Aib-Ala-Phe-TriAzLys*3-PyrS2-Phe-34ClF-sAla*3-Ala-NH2 |
| I-1094 | Ac-PL3-Asp-Ile-B5-Asp-3COOHF-Aib-Ala-Phe-TriAzLys*3-PyrS2-Phe-34ClF-sAla*3-NH2 |
| I-1095 | Ac-PL3-Asp-Ile-B5-Asp-3COOHF-Aib-Ala-Phe-TriAzLys*3-PyrS2-Phe-34ClF-sAla*3-Ala-NH2 |
| I-1096 | Ac-PL3-Asp-Ile-B5-Asp-3COOHF-Phe-Ala-Phe-TriAzLys*3-PyrS2-Phe-34ClF-sAla*3-NH2 |
| I-1097 | Ac-PL3-Asp-Ile-B5-Asp-3COOHF-Phe-Ala-Phe-TriAzLys*3-PyrS2-Phe-34ClF-sAla*3-Ala-NH2 |
| I-1098 | Ac-PL3-Asp-Npg-B5-Asp-3COOHF-Aib-Ala-Phe-Lys*3-PyrS2-3Thi-34MeF-GlnR*3-NH2 |
| I-1099 | Ac-PL3-Asp-Ile-B5-Asp-3COOHF-Aib-Ala-Phe-Lys*3-PyrS2-3Thi-34MeF-GlnR*3-Ala-NH2 |
| I-1100 | Ac-PL3-Asp-Ile-B5-Asp-3COOHF-Aib-Ala-Phe-Lys*3-PyrS2-3Thi-34MeF-GlnR*3-NH2 |
| I-1101 | Ac-PL3-Asp-Ile-B5-Asp-3COOHF-Phe-Ala-Phe-Lys*3-PyrS2-3Thi-34MeF-GlnR*3-Ala-NH2 |
| I-1102 | Ac-PL3-Asp-Ile-B5-Asp-3COOHF-Phe-Ala-Phe-Lys*3-PyrS2-3Thi-34MeF-GlnR*3-NH2 |
| I-1103 | Ac-PL3-Asp-Npg-B5-Asp-3COOHF-Aib-Ala-Phe-Lys*3-PyrS2-Phe-34MeF-GlnR*3-NH2 |
| I-1104 | Ac-PL3-Asp-Ile-B5-Asp-3COOHF-Aib-Ala-Phe-Lys*3-PyrS2-Phe-34MeF-GlnR*3-Ala-NH2 |
| I-1105 | Ac-PL3-Asp-Ile-B5-Asp-3COOHF-Aib-Ala-Phe-Lys*3-PyrS2-Phe-34MeF-GlnR*3-NH2 |
| I-1106 | Ac-PL3-Asp-Ile-B5-Asp-3COOHF-Phe-Ala-Phe-Lys*3-PyrS2-Phe-34MeF-GlnR*3-Ala-NH2 |
| I-1107 | Ac-PL3-Asp-Ile-B5-Asp-3COOHF-Phe-Ala-Phe-Lys*3-PyrS2-Phe-34MeF-GlnR*3-NH2 |
| I-1108 | Ac-PL3-Asp-DipA-B5-Asp-3COOHF-Aib-Ala-Phe-Lys*3-PyrS2-3Thi-34ClF-GlnR*3-NH2 |
| I-1109 | Ac-PL3-Asp-DipA-B5-Asp-3COOHF-Aib-Ala-Phe-Lys*3-PyrS2-3Thi-34ClF-GlnR*3-Ala-NH2 |
| I-1110 | Ac-PL3-Asp-DipA-B5-Asp-3COOHF-Aib-Ala-Phe-Lys*3-PyrS2-3Thi-34ClF-GlnR*3-Ser-NH2 |
| I-1111 | Ac-PL3-Asp-DipA-B5-Asp-3COOHF-Ala-Ala-Phe-Lys*3-PyrS2-3Thi-34ClF-GlnR*3-NH2 |
| I-1112 | Ac-PL3-Asp-DipA-B5-Asp-3COOHF-Ala-Ala-Phe-Lys*3-PyrS2-3Thi-34ClF-GlnR*3-Ala-NH2 |
| I-1113 | Ac-PL3-Asp-DipA-B5-Asp-3COOHF-Ala-Ala-Phe-Lys*3-PyrS2-3Thi-34ClF-GlnR*3-Ser-NH2 |
| I-1114 | Ac-PL3-Asp-DipA-B5-Asp-3COOHF-Phe-Ala-Phe-Lys*3-PyrS2-3Thi-34ClF-GlnR*3-NH2 |
| I-1115 | Ac-PL3-Asp-DipA-B5-Asp-3COOHF-Phe-Ala-Phe-Lys*3-PyrS2-3Thi-34ClF-GlnR*3-Ala-NH2 |
| I-1116 | Ac-PL3-Asp-DipA-B5-Asp-3COOHF-Phe-Ala-Phe-Lys*3-PyrS2-3Thi-34ClF-GlnR*3-Ser-NH2 |
| I-1117 | Ac-PL3-Asp-DipA-B5-Asp-3COOHF-CyLeu-Ala-Phe-Lys*3-PyrS2-3Thi-34ClF-GlnR*3-NH2 |
| I-1118 | Ac-PL3-Asp-DipA-B5-Asp-3COOHF-CyLeu-Ala-Phe-Lys*3-PyrS2-3Thi-34ClF-GlnR*3-Ala-NH2 |
| I-1119 | Ac-PL3-Asp-DipA-B5-Asp-3COOHF-CyLeu-Ala-Phe-Lys*3-PyrS2-3Thi-34ClF-GlnR*3-Ser-NH2 |
| I-1120 | Ac-PL3-Asp-Npg-B5-Asp-3COOHF-Aib-Ala-Phe-TriAzLys*3-PyrSc72SMe3ROMe-3Thi-BztA- |
| | sAla*3-Ala-NH2 |
| I-1121 | Ac-PL3-Asp-Npg-B5-Asp-3COOHF-Aib-Ala-Phe-TriAzLys*3-PyrSc72RMe3SOMe-3Thi-BztA- |
| | sAla*3-Ala-NH2 |
| I-1122 | Ac-PL3-Asp-Npg-B5-Asp-3COOHF-Aib-Ala-Phe-TriAzLys*3-[SMeIso2]PyrSc704-3Thi-BztA- |
| | sAla*3-Ala-NH2 |
| I-1123 | Ac-PL3-Asp-Npg-B5-Asp-3COOHF-Aib-Ala-Phe-TriAzLys*3-[RMeIso2]PyrSc704-3Thi-BztA- |
| | sAla*3-Ala-NH2 |
| I-1124 | Ac-PL3-Asp-Npg-B5-Asp-3COOHF-Aib-Ala-Phe-TriAzLys*3-PyrSc73Me2-3Thi-BztA-sAla*3- |
| | Ala-NH2 |
| I-1125 | Ac-PL3-Asp-Npg-B5-Asp-3COOHF-Aib-Ala-Phe-TriAzLys*3-PyrSc7-3Thi-BztA-sAla*3-Ala- |
| | NH2 |
| I-1126 | Ac-PL3-Asp-Npg-B5-Asp-3COOHF-Aib-Ala-Phe-TriAzLys*3-[CO][Ala]PyrSa-3Thi-BztA- |
| | sAla*3-Ala-NH2 |
| I-1127 | Ac-PL3-Asp-Npg-B5-Asp-3COOHF-Aib-Ala-Phe-TriAzLys*3-[CO][dAla]PyrSa-3Thi-BztA- |
| | sAla*3-Ala-NH2 |
| I-1128 | Ac-S5-Ser-Asp-Npg-B5-Asp-3COOHF-Aib-Ala-Phe-Lys*3-PyrS2-3Thi-BztA-GlnR*3-Ala-NH2 |
| I-1129 | Ac-S5-Ser-Asp-Npg-B5-Asp-3COOHF-Aib-Ala-Phe-Lys*3-PyrS2-3Thi-BztA-GlnR*3-Ala-NH2 |
| I-1130 | Ac-S5-Val-Asp-Npg-B5-Asp-3COOHF-Aib-Ala-Phe-Lys*3-PyrS2-3Thi-BztA-GlnR*3-Ala-NH2 |
| I-1131 | Ac-S5-Val-Asp-Npg-B5-Asp-3COOHF-Aib-Ala-Phe-Lys*3-PyrS2-3Thi-BztA-GlnR*3-Ala-NH2 |
| I-1132 | Ac-S5-Leu-Asp-Npg-B5-Asp-3COOHF-Aib-Ala-Phe-Lys*3-PyrS2-3Thi-BztA-GlnR*3-Ala-NH2 |
| I-1133 | Ac-S5-Leu-Asp-Npg-B5-Asp-3COOHF-Aib-Ala-Phe-Lys*3-PyrS2-3Thi-BztA-GlnR*3-Ala-NH2 |
| I-1134 | Ac-S5-Thr-Asp-Npg-B5-Asp-3COOHF-Aib-Ala-Phe-Lys*3-PyrS2-3Thi-BztA-GlnR*3-Ala-NH2 |
| I-1135 | Ac-S5-Thr-Asp-Npg-B5-Asp-3COOHF-Aib-Ala-Phe-Lys*3-PyrS2-3Thi-BztA-GlnR*3-Ala-NH2 |
| I-1136 | Ac-S5-Phe-Asp-Npg-B5-Asp-3COOHF-Aib-Ala-Phe-Lys*3-PyrS2-3Thi-BztA-GlnR*3-Ala-NH2 |
| I-1137 | Ac-S5-Phe-Asp-Npg-B5-Asp-3COOHF-Aib-Ala-Phe-Lys*3-PyrS2-3Thi-BztA-GlnR*3-Ala-NH2 |
| I-1138 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1139 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1140 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1141 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1142 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1143 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1144 | Ac-S5-Ser-Asp-Npg-B5-Asp-3COOHF-Aib-Ala-Phe-Lys*3-PyrS2-3Thi-BztA-GlnR*3-Ala-NH2 |
| I-1145 | Ac-S5-Val-Asp-Npg-B5-Asp-3COOHF-Aib-Ala-Phe-Lys*3-PyrS2-3Thi-BztA-GlnR*3-Ala-NH2 |
| I-1146 | Ac-S5-Leu-Asp-Npg-B5-Asp-3COOHF-Aib-Ala-Phe-Lys*3-PyrS2-3Thi-BztA-GlnR*3-Ala-NH2 |
| I-1147 | Ac-S5-Thr-Asp-Npg-B5-Asp-3COOHF-Aib-Ala-Phe-Lys*3-PyrS2-3Thi-BztA-GlnR*3-Ala-NH2 |
| I-1148 | Ac-S5-Phe-Asp-Npg-B5-Asp-3COOHF-Aib-Ala-Phe-Lys*3-PyrS2-3Thi-BztA-GlnR*3-Ala-NH2 |
| I-1149 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1150 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1151 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1152 | Ac-PL3-Val-Npg-B5-Asp-3COOHF-Aib-Ala-Phe-Lys*3-PyrS2-3Thi-BztA-GlnR*3-Ala-NH2 |
| I-1153 | Ac-PL3-Npg-Npg-B5-Asp-3COOHF-Aib-Ala-Phe-Lys*3-PyrS2-3Thi-BztA-GlnR*3-Ala-NH2 |
| I-1154 | Ac-PL3-BztA-Npg-B5-Asp-3COOHF-Aib-Ala-Phe-Lys*3-PyrS2-3Thi-BztA-GlnR*3-Ala-NH2 |
| I-1155 | Ac-PL3-Asp-Npg-B5-Asp-3COOHF-Aib-Ala-Phe-Lys*3-PyrS2-3Thi-BztA-GlnR*3-Ala-Ala-Arg- |
| | Ala-Ala-Ala-Ala-NH2 |
| I-1156 | Ac-PL3-Asp-Npg-B5-Asp-3COOHF-Aib-Ala-Phe-Lys*3-PyrS2-3Thi-BztA-GlnR*3-Ala-Arg-Ala- |
| | Ala-Ala-Arg-Ala-NH2 |
| I-1157 | Ac-PL3-Asp-Npg-B5-Asp-3COOHF-Aib-Ala-Phe-Lys*3-PyrS2-3Thi-BztA-GlnR*3-Ala-Ala-Arg- |
| | Ala-Ala-Ala-Arg-NH2 |
| I-1158 | Ac-PL3-Asp-Npg-B5-Asp-3COOHF-Aib-Ala-Phe-Lys*3-PyrS2-3Thi-BztA-GlnR*3-Ala-Ala-Lys- |
| | Ala-Ala-Ala-Lys-NH2 |
| I-1159 | Ac-PL3-Asp-Npg-B5-Asp-3COOHF-Aib-Ala-Phe-Lys*3-PyrS2-3Thi-BztA-GlnR*3-Ala-Ala- |
| | GlnR**3-Ala-Ala-Ala-Lys**3-NH2 |
| I-1160 | Ac-PL3-Asp-Npg-B5-Asp-3COOHF-Aib-Ala-Phe-Lys*3-PyrS2-3Thi-BztA-GlnR*3-Ala-Ala-Ala- |
| | GlnR**3-Ala-Ala-Ala-Lys**3-NH2 |
| I-1161 | Ac-PL3-Asp-Npg-B5-Asp-3COOHF-Aib-Ala-Phe-Lys*3-PyrS2-c6Phe-BztA-GlnR*3-Ala-NH2 |
| I-1162 | Ac-PL3-Asn-Npg-B5-Asp-3COOHF-Aib-Ala-Phe-Lys*3-PyrS2-3Thi-BztA-GlnR*3-NH2 |
| I-1163 | Ac-PL3-Asn-DipA-B5-Asp-3COOHF-Aib-Ala-Phe-Lys*3-PyrS2-3Thi-BztA-GlnR*3-NH2 |
| I-1164 | Ac-PL3-Asn-DipA-B5-Asp-3COOHF-Aib-Ala-Phe-Lys*3-PyrS2-3Thi-BztA-GlnR*3-Ala-NH2 |
| I-1165 | Ac-PL3-Asn-Ile-B5-Asp-3COOHF-Aib-Ala-Phe-Lys*3-PyrS2-3Thi-BztA-GlnR*3-NH2 |
| I-1166 | Ac-PL3-Asn-Ile-B5-Asp-3COOHF-Aib-Ala-Phe-Lys*3-PyrS2-3Thi-BztA-GlnR*3-NH2 |
| I-1167 | Ac-PL3-Asn-Ile-B5-Asp-3COOHF-Aib-Ala-Phe-Lys*3-PyrS2-3Thi-BztA-GlnR*3-Ala-NH2 |
| I-1168 | Ac-PL3-Asn-Ile-B5-Asp-3COOHF-Aib-Ala-Phe-Lys*3-PyrS2-3Thi-BztA-GlnR*3-Ala-NH2 |
| I-1169 | Ac-PL3-Asn-Ile-B5-Asp-3COOHF-Aib-Ala-Phe-Lys*3-PyrS2-3Thi-BztA-GlnR*3-Ser-NH2 |
| I-1170 | Ac-PL3-Asn-Ile-B5-Asp-3COOHF-Aib-Ala-Phe-Lys*3-PyrS2-3Thi-BztA-GlnR*3-Ser-NH2 |
| I-1171 | Ac-PL3-Asn-Npg-B5-Asp-3COOHF-Aib-Ala-Phe-Lys*3-PyrS2-Phe-34ClF-GlnR*3-NH2 |
| I-1172 | Ac-PL3-Asn-Npg-B5-Asp-3COOHF-Aib-Ala-Phe-Lys*3-PyrS2-Phe-34ClF-GlnR*3-NH2 |
| I-1173 | Ac-PL3-Asn-Npg-B5-Asp-3COOHF-Aib-Ala-Phe-Lys*3-PyrS2-Phe-34ClF-GlnR*3-Ala-NH2 |
| I-1174 | Ac-PL3-Asn-Npg-B5-Asp-3COOHF-Aib-Ala-Phe-Lys*3-PyrS2-Phe-34ClF-GlnR*3-Ala-NH2 |
| I-1175 | Ac-PL3-Asn-DipA-B5-Asp-3COOHF-Aib-Ala-Phe-Lys*3-PyrS2-Phe-34ClF-GlnR*3-NH2 |
| I-1176 | Ac-PL3-Asn-DipA-B5-Asp-3COOHF-Aib-Ala-Phe-Lys*3-PyrS2-Phe-34ClF-GlnR*3-NH2 |
| I-1177 | Ac-PL3-Asn-DipA-B5-Asp-3COOHF-Aib-Ala-Phe-Lys*3-PyrS2-Phe-34ClF-GlnR*3-Ala-NH2 |
| I-1178 | Ac-PL3-Asn-Ile-B5-Asp-3COOHF-Aib-Ala-Phe-Lys*3-PyrS2-Phe-34ClF-GlnR*3-NH2 |
| I-1179 | Ac-PL3-Asn-Ile-B5-Asp-3COOHF-Aib-Ala-Phe-Lys*3-PyrS2-Phe-34ClF-GlnR*3-NH2 |
| I-1180 | Ac-PL3-Asn-Ile-B5-Asp-3COOHF-Aib-Ala-Phe-Lys*3-PyrS2-Phe-34ClF-GlnR*3-Ala-NH2 |
| I-1181 | Ac-PL3-Asp-Npg-B5-Asp-3COOHF-Aib-Ala-Phe-Lys*3-PyrS2-3Thi-BztA-GlnR*3-[mPEG4]Lys- |
| | NH2 |
| I-1182 | Ac-PL3-Asp-Npg-B5-Asp-3COOHF-Aib-Ala-Phe-Lys*3-PyrS2-3Thi-BztA-GlnR*3-[mPEG8]Lys- |
| | NH2 |
| I-1183 | Ac-PL3-Asp-Npg-B5-Asp-3COOHF-Aib-Ala-Phe-Lys*3-PyrS2-3Thi-BztA-GlnR*3- |
| | [mPEG16]Lys-NH2 |
| I-1184 | mPEG4-PL3-Asp-Npg-B5-Asp-3COOHF-Aib-Ala-Phe-Lys*3-PyrS2-3Thi-BztA-GlnR*3-Ala-NH2 |
| I-1185 | mPEG8-PL3-Asp-Npg-B5-Asp-3COOHF-Aib-Ala-Phe-Lys*3-PyrS2-3Thi-BztA-GlnR*3-Ala-NH2 |
| I-1186 | mPEG16-PL3-Asp-Npg-B5-Asp-3COOHF-Aib-Ala-Phe-Lys*3-PyrS2-3Thi-BztA-GlnR*3-Ala- |
| | NH2 |
| I-1187 | mPEG24-PL3-Asp-Npg-B5-Asp-3COOHF-Aib-Ala-Phe-Lys*3-PyrS2-3Thi-BztA-GlnR*3-Ala- |
| | NH2 |
| I-1188 | Ac-PL3-Asp-Npg-B5-Asp-3BOH2F-Aib-Ala-Phe-Lys*3-PyrS2-3Thi-BztA-GlnR*3-Ala-NH2 |
| I-1189 | Ac-PL3-Asp-Npg-B5-Asp-4BOH2F-Aib-Ala-Phe-Lys*3-PyrS2-3Thi-BztA-GlnR*3-Ala-NH2 |
| I-1190 | Ac-PL3-Asp-Npg-B5-Asp-4BOH2F-Aib-Ala-Phe-Lys*3-PyrS2-3Thi-BztA-GlnR*3-Ala-NH2 |
| I-1191 | Ac-S5-Ala-Glu-Npg-B5-Asp-3COOHF-Aib-Ala-Phe-Lys*3-PyrS2-3Thi-BztA-GlnR*3-Ala-NH2 |
| I-1192 | Ac-S5-Ala-Glu-Npg-B5-Asp-3COOHF-Aib-Ala-Phe-Lys*3-PyrS2-3Thi-BztA-GlnR*3-Ala-NH2 |
| I-1193 | Ac-S5-Ala-Asn-Npg-B5-Asp-3COOHF-Aib-Ala-Phe-Lys*3-PyrS2-3Thi-BztA-GlnR*3-Ala-NH2 |
| I-1194 | Ac-S5-Ala-Asn-Npg-B5-Asp-3COOHF-Aib-Ala-Phe-Lys*3-PyrS2-3Thi-BztA-GlnR*3-Ala-NH2 |
| I-1195 | Ac-S5-Ala-Ser-Npg-B5-Asp-3COOHF-Aib-Ala-Phe-Lys*3-PyrS2-3Thi-BztA-GlnR*3-Ala-NH2 |
| I-1196 | Ac-S5-Ala-Ser-Npg-B5-Asp-3COOHF-Aib-Ala-Phe-Lys*3-PyrS2-3Thi-BztA-GlnR*3-Ala-NH2 |
| I-1197 | Ac-S5-Ala-Hse-Npg-B5-Asp-3COOHF-Aib-Ala-Phe-Lys*3-PyrS2-3Thi-BztA-GlnR*3-Ala-NH2 |
| I-1198 | Ac-S5-Ala-Hse-Npg-B5-Asp-3COOHF-Aib-Ala-Phe-Lys*3-PyrS2-3Thi-BztA-GlnR*3-Ala-NH2 |
| I-1199 | Ac-S5-Ala-Asp-Npg-B5-Glu-3COOHF-Aib-Ala-Phe-Lys*3-PyrS2-3Thi-BztA-GlnR*3-Ala-NH2 |
| I-1200 | Ac-S5-Ala-Asp-Npg-B5-Glu-3COOHF-Aib-Ala-Phe-Lys*3-PyrS2-3Thi-BztA-GlnR*3-Ala-NH2 |
| I-1201 | Ac-S5-Ala-Asp-Npg-B5-Asn-3COOHF-Aib-Ala-Phe-Lys*3-PyrS2-3Thi-BztA-GlnR*3-Ala-NH2 |
| I-1202 | Ac-S5-Ala-Asp-Npg-B5-Asn-3COOHF-Aib-Ala-Phe-Lys*3-PyrS2-3Thi-BztA-GlnR*3-Ala-NH2 |
| I-1203 | Ac-S5-Ala-Asp-Npg-B5-Ser-3COOHF-Aib-Ala-Phe-Lys*3-PyrS2-3Thi-BztA-GlnR*3-Ala-NH2 |
| I-1204 | Ac-S5-Ala-Asp-Npg-B5-Ser-3COOHF-Aib-Ala-Phe-Lys*3-PyrS2-3Thi-BztA-GlnR*3-Ala-NH2 |
| I-1205 | Ac-S5-Ala-Asp-Npg-B5-Hse-3COOHF-Aib-Ala-Phe-Lys*3-PyrS2-3Thi-BztA-GlnR*3-Ala-NH2 |
| I-1206 | Ac-S5-Ala-Asp-Npg-B5-Hse-3COOHF-Aib-Ala-Phe-Lys*3-PyrS2-3Thi-BztA-GlnR*3-Ala-NH2 |
| I-1207 | Ac-S5-Ala-Glu-Npg-B5-Glu-3COOHF-Aib-Ala-Phe-Lys*3-PyrS2-3Thi-BztA-GlnR*3-Ala-NH2 |
| I-1208 | Ac-S5-Ala-Glu-Npg-B5-Glu-3COOHF-Aib-Ala-Phe-Lys*3-PyrS2-3Thi-BztA-GlnR*3-Ala-NH2 |
| I-1209 | Ac-S5-Glu-Glu-Npg-B5-Asp-3COOHF-Aib-Ala-Phe-Lys*3-PyrS2-3Thi-BztA-GlnR*3-Ala-NH2 |
| I-1210 | Ac-S5-Glu-Glu-Npg-B5-Asp-3COOHF-Aib-Ala-Phe-Lys*3-PyrS2-3Thi-BztA-GlnR*3-Ala-NH2 |
| I-1211 | Ac-S5-Glu-Asp-Npg-B5-Glu-3COOHF-Aib-Ala-Phe-Lys*3-PyrS2-3Thi-BztA-GlnR*3-Ala-NH2 |
| I-1212 | Ac-S5-Glu-Asp-Npg-B5-Glu-3COOHF-Aib-Ala-Phe-Lys*3-PyrS2-3Thi-BztA-GlnR*3-Ala-NH2 |
| I-1213 | Ac-PL3-Asp-Npg-B5-Asp-3COOHF-Ala-Ala-Phe-Leu-[Red][Gly]Dap-3thi-BztA-Ala-GlnR-NH2 |
| I-1214 | Ac-PL3-Asp-Npg-B5-Asp-3COOHF-Ala-Ala-Phe-Leu-[Red][NHPent]Dap-3thi-BztA-Ala-GlnR- |
| | NH2 |
| I-1215 | Ac-PL3-Asp-Npg-B5-Asp-3COOHF-Ala-Ala-Phe-Leu-[SBut][CH2CH2NH]Dap-3thi-BztA-Ala- |
| | GlnR-NH2 |
| I-1216 | Ac-PL3-Asp-Npg-B5-Asp-3COOHF-Ala-Ala-Phe-Leu-[SBut][CH2CH2CH2NH]Dap-3thi-BztA- |
| | Ala-GlnR-NH2 |
| I-1217 | Ac-S5-Ala-Glu-Npg-B5-Asp-3COOHF-Aib-Ala-Phe-Lys*3-PyrS2-3Thi-BztA-GlnR*3-Ala-NH2 |
| I-1218 | Ac-S5-Ala-Asn-Npg-B5-Asp-3COOHF-Aib-Ala-Phe-Lys*3-PyrS2-3Thi-BztA-GlnR*3-Ala-NH2 |
| I-1219 | Ac-S5-Ala-Ser-Npg-B5-Asp-3COOHF-Aib-Ala-Phe-Lys*3-PyrS2-3Thi-BztA-GlnR*3-Ala-NH2 |
| I-1220 | Ac-S5-Ala-Hse-Npg-B5-Asp-3COOHF-Aib-Ala-Phe-Lys*3-PyrS2-3Thi-BztA-GlnR*3-Ala-NH2 |
| I-1221 | Ac-S5-Ala-Asp-Npg-B5-Glu-3COOHF-Aib-Ala-Phe-Lys*3-PyrS2-3Thi-BztA-GlnR*3-Ala-NH2 |
| I-1222 | Ac-S5-Ala-Asp-Npg-B5-Asn-3COOHF-Aib-Ala-Phe-Lys*3-PyrS2-3Thi-BztA-GlnR*3-Ala-NH2 |
| I-1223 | Ac-S5-Ala-Asp-Npg-B5-Ser-3COOHF-Aib-Ala-Phe-Lys*3-PyrS2-3Thi-BztA-GlnR*3-Ala-NH2 |
| I-1224 | Ac-S5-Ala-Asp-Npg-B5-Hse-3COOHF-Aib-Ala-Phe-Lys*3-PyrS2-3Thi-BztA-GlnR*3-Ala-NH2 |
| I-1225 | Ac-S5-Ala-Glu-Npg-B5-Glu-3COOHF-Aib-Ala-Phe-Lys*3-PyrS2-3Thi-BztA-GlnR*3-Ala-NH2 |
| I-1226 | Ac-S5-Glu-Glu-Npg-B5-Asp-3COOHF-Aib-Ala-Phe-Lys*3-PyrS2-3Thi-BztA-GlnR*3-Ala-NH2 |
| I-1227 | Ac-S5-Glu-Asp-Npg-B5-Glu-3COOHF-Aib-Ala-Phe-Lys*3-PyrS2-3Thi-BztA-GlnR*3-Ala-NH2 |
| I-1228 | Ac-S5-Glu-Glu-Npg-B5-Glu-3COOHF-Aib-Ala-Phe-Lys*3-PyrS2-3Thi-BztA-GlnR*3-Ala-NH2 |
| I-1229 | Ac-S5-Ala-Asp-Npg-B5-Asp-3COOHF-Aib-Ala-Phe-Lys*3-PyrS2-Phe-34ClF-GlnR*3-Ala-NH2 |
| I-1230 | Ac-S5-Ala-Asp-Npg-B5-Asp-3COOHF-Aib-Ala-Phe-Lys*3-PyrS2-Phe-34ClF-GlnR*3-NH2 |
| I-1231 | Ac-S5-Ala-Asp-Npg-B5-Asp-3COOHF-Aib-Ala-Phe-Lys*3-PyrS2-3Thi-34ClF-GlnR*3-Ala-NH2 |
| I-1232 | Ac-S5-Ala-Asp-Npg-B5-Asp-3COOHF-Aib-Ala-Phe-Lys*3-PyrS2-3Thi-34ClF-GlnR*3-NH2 |
| I-1233 | Ac-S5-Ala-Asp-Ile-B5-Asp-3COOHF-Aib-Ala-Phe-Lys*3-PyrS2-Phe-34ClF-GlnR*3-Ala-NH2 |
| I-1234 | Ac-S5-Ala-Asp-Ile-B5-Asp-3COOHF-Aib-Ala-Phe-Lys*3-PyrS2-Phe-34ClF-GlnR*3-NH2 |
| I-1235 | Ac-S5-Ala-Asp-Ala-B5-Asp-3COOHF-Aib-Ala-Phe-Lys*3-PyrS2-Phe-34ClF-GlnR*3-Ala-NH2 |
| I-1236 | Ac-S5-Ala-Asp-Ala-B5-Asp-3COOHF-Aib-Ala-Phe-Lys*3-PyrS2-Phe-34ClF-GlnR*3-NH2 |
| I-1237 | Ac-S5-Ala-Asp-Phe-B5-Asp-3COOHF-Aib-Ala-Phe-Lys*3-PyrS2-Phe-34ClF-GlnR*3-Ala-NH2 |
| I-1238 | Ac-S5-Ala-Asp-Phe-B5-Asp-3COOHF-Aib-Ala-Phe-Lys*3-PyrS2-Phe-34ClF-GlnR*3-NH2 |
| I-1239 | Ac-S5-Ala-Asp-Ile-B5-Asp-3COOHF-Ala-Ala-Phe-Lys*3-PyrS2-Phe-34ClF-GlnR*3-Ala-NH2 |
| I-1240 | Ac-S5-Ala-Asp-Ile-B5-Asp-3COOHF-Ala-Ala-Phe-Lys*3-PyrS2-Phe-34ClF-GlnR*3-NH2 |
| I-1241 | Ac-PL3-Asp-Ile-B5-Asp-3COOHF-[isovaleryl]Acp-Ala-Phe-TriAzLys*3-PyrS2-Phe-34ClF- |
| | sAla*3-NH2 |
| I-1242 | Ac-PL3-Asp-Npg-B5-Asp-3COOHF-Aib-Ala-Phe-Lys*3-PyrS2-Phe-BztA-GlnR*3-NH2 |
| I-1243 | Ac-PL3-Asp-Npg-B5-Asp-3COOHF-Ala-Ala-Phe-Lys*3-PyrS2-Phe-BztA-GlnR*3-NH2 |
| I-1244 | Ac-PL3-Asp-Npg-B5-Asp-3COOHF-Ala-Ala-Phe-Lys*3-PyrS2-Phe-BztA-GlnR*3-Ala-NH2 |
| I-1245 | Ac-PL3-Asp-Npg-B5-Asp-3COOHF-Aib-Ala-Phe-Lys*3-PyrS2-Phe-7FBztA-GlnR*3-NH2 |
| I-1246 | Ac-PL3-Asp-Npg-B5-Asp-3COOHF-Aib-Ala-Phe-Lys*3-PyrS2-Phe-7FBztA-GlnR*3-NH2 |
| I-1247 | Ac-PL3-Asp-Npg-B5-Asp-3COOHF-Aib-Ala-Phe-Lys*3-PyrS2-Phe-7FBztA-GlnR*3-Ala-NH2 |
| I-1248 | Ac-PL3-Asp-Npg-B5-Asp-3COOHF-Aib-Ala-Phe-Lys*3-PyrS2-Phe-7ClBztA-GlnR*3-NH2 |
| I-1249 | Ac-PL3-Asp-Npg-B5-Asp-3COOHF-Aib-Ala-Phe-Lys*3-PyrS2-Phe-7ClBztA-GlnR*3-Ala-NH2 |
| I-1250 | Ac-PL3-Asp-Npg-B5-Asp-3COOHF-Aib-Ala-Phe-Lys*3-PyrS2-Phe-7MeBztA-GlnR*3-NH2 |
| I-1251 | Ac-PL3-Asp-Npg-B5-Asp-3COOHF-Aib-Ala-Phe-Lys*3-PyrS2-Phe-7MeBztA-GlnR*3-Ala-NH2 |
| I-1252 | Ac-PL3-Asp-Ile-B5-Asp-3COOHF-Aib-Ala-Phe-Lys*3-PyrS2-Phe-34ClF-GlnR*3-Val-Glu-Ala- |
| | NH2 |
| I-1253 | Ac-PL3-Asp-Ile-B5-Asp-3COOHF-Aib-Ala-Phe-Lys*3-PyrS2-Phe-34ClF-GlnR*3-Val-Glu-Ala- |
| | NH2 |
| I-1254 | Ac-PL3-Asp-Ile-B5-Asp-3COOHF-Aib-Ala-Phe-Lys*3-PyrS2-Phe-34ClF-GlnR*3-Val-Glu-Leu- |
| | NH2 |
| I-1255 | Ac-PL3-Asp-Ile-B5-Asp-3COOHF-Aib-Ala-Phe-Lys*3-PyrS2-Phe-34ClF-GlnR*3-Val-Glu-Leu- |
| | NH2 |
| I-1256 | Ac-PL3-Asp-Ile-B5-Asp-3COOHF-Aib-Ala-Phe-Lys*3-PyrS2-Phe-34ClF-GlnR*3-Val-Thr-Ala- |
| | NH2 |
| I-1257 | Ac-PL3-Asp-Ile-B5-Asp-3COOHF-Aib-Ala-Phe-Lys*3-PyrS2-Phe-34ClF-GlnR*3-Val-Thr-Ala- |
| | NH2 |
| I-1258 | Ac-PL3-Asp-Ile-B5-Asp-3COOHF-CyLeu-Ala-Phe-Lys*3-PyrS2-Phe-34ClF-GlnR*3-Val-Glu-Ala- |
| | NH2 |
| I-1259 | Ac-PL3-Asp-Ile-B5-Asp-3COOHF-CyLeu-Ala-Phe-Lys*3-PyrS2-Phe-34ClF-GlnR*3-Val-Glu-Ala- |
| | NH2 |
| I-1260 | Ac-PL3-Asp-Ile-B5-Asp-3COOHF-CyLeu-Ala-Phe-Lys*3-PyrS2-Phe-34ClF-GlnR*3-Val-Glu-Leu- |
| | NH2 |
| I-1261 | Ac-PL3-Asp-Ile-B5-Asp-3COOHF-CyLeu-Ala-Phe-Lys*3-PyrS2-Phe-34ClF-GlnR*3-Val-Glu-Leu- |
| | NH2 |
| I-1262 | Ac-PL3-Asp-Ile-B5-Asp-3COOHF-CyLeu-Ala-Phe-Lys*3-PyrS2-Phe-34ClF-GlnR*3-Val-Thr-Ala- |
| | NH2 |
| I-1263 | Ac-PL3-Asp-Ile-B5-Asp-3COOHF-Aib-Ala-Phe-Lys*3-PyrS2-3Thi-34ClF-GlnR*3-Val-Glu-Ala- |
| | NH2 |
| I-1264 | Ac-PL3-Asp-Ile-B5-Asp-3COOHF-Aib-Ala-Phe-Lys*3-PyrS2-3Thi-34ClF-GlnR*3-Val-Glu-Leu- |
| | NH2 |
| I-1265 | Ac-PL3-Asp-Ile-B5-Asp-3COOHF-Aib-Ala-Phe-Lys*3-PyrS2-3Thi-34ClF-GlnR*3-Val-Thr-Ala- |
| | NH2 |
| I-1266 | Ac-PL3-Asp-Ile-B5-Asp-3COOHF-CyLeu-Ala-Phe-Lys*3-PyrS2-3Thi-34ClF-GlnR*3-Val-Glu- |
| | Ala-NH2 |
| I-1267 | Ac-PL3-Asp-Ile-B5-Asp-3COOHF-CyLeu-Ala-Phe-Lys*3-PyrS2-3Thi-34ClF-GlnR*3-Val-Glu- |
| | Leu-NH2 |
| I-1268 | Ac-PL3-Asp-Ile-B5-Asp-3COOHF-CyLeu-Ala-Phe-Lys*3-PyrS2-3Thi-34ClF-GlnR*3-Val-Thr- |
| | Ala-NH2 |
| I-1269 | Ac-PL3-Asp-Npg-B5-Asp-3COOHF-Aib-Ala-Phe-[3_3-biPh]hCys-PyrS2-3Thi-BztA-Cys-Ala-NH2 |
| I-1270 | Ac-PL3-Asp-Npg-B5-Asp-3COOHF-Aib-Ala-Phe-[2_6-naph]hCys-PyrS2-3Thi-BztA-Cys-Ala- |
| | NH2 |
| I-1271 | Ac-PL3-Asp-Npg-B5-Asp-3COOHF-Aib-Ala-Phe-[mPyr]Cys-PyrS2-3Thi-BztA-Cys-Ala-NH2 |
| I-1272 | Ac-PL3-Asp-Npg-B5-Asp-3COOHF-Aib-Ala-Phe-[mXyl]Cys-PyrS2-3Thi-BztA-Cys-Ala-NH2 |
| I-1273 | Ac-PL3-Asp-Npg-B5-Asp-3COOHF-Aib-Ala-Phe-[mPyr]hCys-PyrS2-3Thi-BztA-Cys-Ala-NH2 |
| I-1274 | Ac-PL3-Asp-Npg-B5-Asp-3COOHF-Aib-Ala-Phe-[C3]Cys-PyrS2-3Thi-BztA-Cys-Ala-NH2 |
| I-1275 | Ac-PL3-Asp-Npg-B5-Asp-3COOHF-Aib-Ala-Phe-[Red]Cys-PyrS2-3Thi-BztA-Cys-Ala-NH2 |
| I-1276 | Ac-PL3-Asp-Npg-B5-Asp-3COOHF-Aib-Ala-Phe-[33Oxe]hCys-PyrS2-3Thi-BztA-Cys-Ala-NH2 |
| I-1277 | Ac-PL3-Asp-Npg-B5-Asp-3COOHF-Aib-Ala-Phe-[33Oxe]Cys-PyrS2-3Thi-BztA-Cys-Ala-NH2 |
| I-1278 | Ac-PL3-Asp-Npg-B5-Asp-3COOHF-Aib-Ala-Phe-[IsoE]Cys-PyrS2-3Thi-BztA-Cys-Ala-NH2 |
| I-1279 | Ac-PL3-Asp-Npg-B5-Asp-3COOHF-Aib-Ala-Phe-[C3]hCys-PyrS2-3Thi-BztA-Cys-Ala-NH2 |
| I-1280 | Ac-PL3-Asp-Npg-B5-Asp-3COOHF-Aib-Ala-Phe-[Red]hCys-PyrS2-3Thi-BztA-Cys-Ala-NH2 |
| I-1281 | Ac-PL3-Asp-Npg-B5-Asp-3COOHF-Aib-Ala-Phe-[IsoE]hCys-PyrS2-3Thi-BztA-Cys-Ala-NH2 |
| I-1282 | Ac-PL3-Asp-Npg-B5-Asp-3COOHF-Aib-Ala-Phe-[13Ac]Cys-PyrS2-3Thi-BztA-Cys-Ala-NH2 |
| I-1283 | Ac-PL3-Asp-Npg-B5-Asp-3COOHF-Aib-Ala-Phe-[13Ac]hCys-PyrS2-3Thi-BztA-Cys-Ala-NH2 |
| I-1284 | Ac-Ala-Ala-S5-Ala-Asp-Npg-B5-Asp-3COOHF-Aib-Ala-Phe-Lys*3-PyrS2-3Thi-BztA-GlnR*3- |
| | Ala |
| I-1285 | Ac-Asp-Ala-S5-Ala-Asp-Npg-B5-Asp-3COOHF-Aib-Ala-Phe-Lys*3-PyrS2-3Thi-BztA-GlnR*3- |
| | Ala |
| I-1286 | Ac-Pro-Ala-S5-Ala-Asp-Npg-B5-Asp-3COOHF-Aib-Ala-Phe-Lys*3-PyrS2-3Thi-BztA-GlnR*3- |
| | Ala |
| I-1287 | Ac-Ala-Ala-S5-Ser-Asp-Npg-B5-Asp-3COOHF-Aib-Ala-Phe-Lys*3-PyrS2-3Thi-BztA-GlnR*3-Ala |
| I-1288 | Ac-Asp-Ala-S5-Ser-Asp-Npg-B5-Asp-3COOHF-Aib-Ala-Phe-Lys*3-PyrS2-3Thi-BztA-GlnR*3- |
| | Ala |
| I-1289 | Ac-Pro-Ala-S5-Ser-Asp-Npg-B5-Asp-3COOHF-Aib-Ala-Phe-Lys*3-PyrS2-3Thi-BztA-GlnR*3-Ala |
| I-1290 | Ac-Ala-Ala-S5-Leu-Asp-Npg-B5-Asp-3COOHF-Aib-Ala-Phe-Lys*3-PyrS2-3Thi-BztA-GlnR*3- |
| | Ala |
| I-1291 | Ac-Asp-Ala-S5-Leu-Asp-Npg-B5-Asp-3COOHF-Aib-Ala-Phe-Lys*3-PyrS2-3Thi-BztA-GlnR*3- |
| | Ala |
| I-1292 | Ac-Pro-Ala-S5-Leu-Asp-Npg-B5-Asp-3COOHF-Aib-Ala-Phe-Lys*3-PyrS2-3Thi-BztA-GlnR*3- |
| | Ala |
| I-1293 | Ac-Ala-Ser-S5-Ala-Asp-Npg-B5-Asp-3COOHF-Aib-Ala-Phe-Lys*3-PyrS2-3Thi-BztA-GlnR*3-Ala |
| I-1294 | Ac-Asp-Ser-S5-Ala-Asp-Npg-B5-Asp-3COOHF-Aib-Ala-Phe-Lys*3-PyrS2-3Thi-BztA-GlnR*3- |
| | Ala |
| I-1295 | Ac-Pro-Ser-S5-Ala-Asp-Npg-B5-Asp-3COOHF-Aib-Ala-Phe-Lys*3-PyrS2-3Thi-BztA-GlnR*3-Ala |
| I-1296 | Ac-S5-Leu-Asp-Ile-B5-Asp-3COOHF-Aib-Ala-Phe-Lys*3-PyrS2-Phe-34ClF-GlnR*3-NH2 |
| I-1297 | Ac-S5-Ala-Asp-Npg-B5-Ala-Asp-3COOHF-Aib-Ala-Phe-[4Abu]DapAc7-Leu-3Thi-BztA-GlnR*3- |
| | Ala-NH2 |
| I-1298 | Ac-S6-Ala-Asp-Npg-B5-Ala-Asp-3COOHF-Aib-Ala-Phe-[4Abu]DapAc7-Leu-3Thi-BztA-GlnR*3- |
| | Ala-NH2 |
| I-1299 | Ac-S5-Ala-Asp-Npg-B5-Asp-3COOHF-Aib-Ala-Phe-Leu-[4Abu]DapAc7-3thi-BztA-Ala-GlnR*3- |
| | NH2 |
| I-1300 | Ac-S6-Ala-Asp-Npg-B5-Asp-3COOHF-Aib-Ala-Phe-Leu-[4Abu]DapAc7-3thi-BztA-Ala-GlnR*3- |
| | NH2 |
| I-1301 | Ac-PL3-AspSH-Npg-B5-Asp-3COOHF-Aib-Ala-Phe-Lys*3-PyrS2-3Thi-BztA-GlnR*3-Ala-NH2 |
| I-1302 | Ac-PL3-Asp-Npg-B5-AspSH-3COOHF-Aib-Ala-Phe-Lys*3-PyrS2-3Thi-BztA-GlnR*3-Ala-NH2 |
| I-1303 | Ac-PL3-AspSH-Npg-B5-AspSH-3COOHF-Aib-Ala-Phe-Lys*3-PyrS2-3Thi-BztA-GlnR*3-Ala- |
| | NH2 |
| I-1304 | Ac-PL3-Asp-Npg-B5-Asp-3COOHF-Aib-Ala-Phe-Lys*3-PyrS2-hhLeu-BztA-GlnR*3-Ala-NH2 |
| I-1305 | Ac-PL3-Asp-Npg-B5-Asp-3COOHF-Aib-Ala-Phe-Lys*3-PyrS2-hPhe-BztA-GlnR*3-Ala-NH2 |
| I-1306 | Ac-PL3-Asp-Npg-B5-Asp-3COOHF-Aib-Ala-Phe-Lys*3-PyrS2-hhSer-BztA-GlnR*3-Ala-NH2 |
| I-1307 | Ac-PL3-Asp-Npg-B5-Asp-3COOHF-Aib-Ala-Phe-Lys*3-PyrS2-hhLeu-34ClF-GlnR*3-Ala-NH2 |
| I-1308 | Ac-PL3-Asp-Npg-B5-Asp-3COOHF-Aib-Ala-Phe-Lys*3-PyrS2-hPhe-34ClF-GlnR*3-Ala-NH2 |
| I-1309 | Ac-PL3-Asp-Npg-B5-Asp-3COOHF-Aib-Ala-Phe-Lys*3-PyrS2-hhSer-34ClF-GlnR*3-Ala-NH2 |
| I-1310 | Ac-PL3-Asp-Ile-B5-Asp-3COOHF-Aib-Ala-Phe-Lys*3-PyrS2-hhLeu-BztA-GlnR*3-Ala-NH2 |
| I-1311 | Ac-PL3-Asp-Ile-B5-Asp-3COOHF-Aib-Ala-Phe-Lys*3-PyrS2-hPhe-BztA-GlnR*3-Ala-NH2 |
| I-1312 | Ac-PL3-Asp-Ile-B5-Asp-3COOHF-Aib-Ala-Phe-Lys*3-PyrS2-hPhe-BztA-GlnR*3-Ala-NH2 |
| I-1313 | Ac-PL3-Asp-Ile-B5-Asp-3COOHF-Aib-Ala-Phe-Lys*3-PyrS2-hhSer-BztA-GlnR*3-Ala-NH2 |
| I-1314 | Ac-PL3-Asp-Ile-B5-Asp-3COOHF-Aib-Ala-Phe-Lys*3-PyrS2-hhLeu-34ClF-GlnR*3-Ala-NH2 |
| I-1315 | Ac-PL3-Asp-Ile-B5-Asp-3COOHF-Aib-Ala-Phe-Lys*3-PyrS2-hPhe-34ClF-GlnR*3-Ala-NH2 |
| I-1316 | Ac-PL3-Asp-Ile-B5-Asp-3COOHF-Aib-Ala-Phe-Lys*3-PyrS2-hhSer-34ClF-GlnR*3-Ala-NH2 |
| I-1317 | Ac-PL3-Asp-Npg-B5-Asp-3COOHF-Aib-Ala-Phe-Lys*3-PyrS2-hCbA-BztA-GlnR*3-Ala-NH2 |
| I-1318 | Ac-PL3-Asp-Npg-B5-Asp-3COOHF-Aib-Ala-Phe-Lys*3-PyrS2-hCypA-BztA-GlnR*3-Ala-NH2 |
| I-1319 | Ac-PL3-Asp-Npg-B5-Asp-3COOHF-Aib-Ala-Phe-Lys*3-PyrS2-hCha-BztA-GlnR*3-Ala-NH2 |
| I-1320 | Ac-PL3-Asp-Npg-B5-Asp-3COOHF-Aib-Ala-Phe-Lys*3-PyrS2-hCbA-34ClF-GlnR*3-Ala-NH2 |
| I-1321 | Ac-PL3-Asp-Npg-B5-Asp-3COOHF-Aib-Ala-Phe-Lys*3-PyrS2-hCypA-34ClF-GlnR*3-Ala-NH2 |
| I-1322 | Ac-PL3-Asp-Npg-B5-Asp-3COOHF-Aib-Ala-Phe-Lys*3-PyrS2-hCha-34ClF-GlnR*3-Ala-NH2 |
| I-1323 | Ac-PL3-Asp-Ile-B5-Asp-3COOHF-Aib-Ala-Phe-Lys*3-PyrS2-hCbA-BztA-GlnR*3-Ala-NH2 |
| I-1324 | Ac-PL3-Asp-Ile-B5-Asp-3COOHF-Aib-Ala-Phe-Lys*3-PyrS2-hCypA-BztA-GlnR*3-Ala-NH2 |
| I-1325 | Ac-PL3-Asp-Ile-B5-Asp-3COOHF-Aib-Ala-Phe-Lys*3-PyrS2-hCha-BztA-GlnR*3-Ala-NH2 |
| I-1326 | Ac-PL3-Asp-Ile-B5-Asp-3COOHF-Aib-Ala-Phe-Lys*3-PyrS2-hCbA-34ClF-GlnR*3-Ala-NH2 |
| I-1327 | Ac-PL3-Asp-Ile-B5-Asp-3COOHF-Aib-Ala-Phe-Lys*3-PyrS2-hCypA-34ClF-GlnR*3-Ala-NH2 |
| I-1328 | Ac-PL3-Asp-Ile-B5-Asp-3COOHF-Aib-Ala-Phe-Lys*3-PyrS2-hCha-34ClF-GlnR*3-Ala-NH2 |
| I-1329 | Ac-S5-Ala-Asp-Npg-B5-Asp-Asp-Aib-Ala-Phe-Lys*3-PyrS2-3Thi-BztA-GlnR*3-Ala-NH2 |
| I-1330 | Ac-S5-Ala-Asp-Npg-B5-Glu-Asp-Aib-Ala-Phe-Lys*3-PyrS2-3Thi-BztA-GlnR*3-Ala-NH2 |
| I-1331 | Ac-S5-Ala-Asp-Npg-B5-TfeGA-Asp-Aib-Ala-Phe-Lys*3-PyrS2-3Thi-BztA-GlnR*3-Ala-NH2 |
| I-1332 | Ac-S5-Pro-Asp-Npg-B5-Asp-Asp-Aib-Ala-Phe-Lys*3-PyrS2-3Thi-BztA-GlnR*3-Ala-NH2 |
| I-1333 | Ac-S5-Pro-Asp-Npg-B5-Glu-Asp-Aib-Ala-Phe-Lys*3-PyrS2-3Thi-BztA-GlnR*3-Ala-NH2 |
| I-1334 | Ac-S5-Pro-Asp-Npg-B5-TfeGA-Asp-Aib-Ala-Phe-Lys*3-PyrS2-3Thi-BztA-GlnR*3-Ala-NH2 |
| I-1335 | Ac-S5-Ala-Asp-Ile-B5-Asp-2COOHF-Aib-Ala-Phe-Lys*3-PyrS2-Phe-34ClF-GlnR*3-NH2 |
| I-1336 | Ac-S5-Ala-Asp-Ile-B5-Asp-4COOHF-Aib-Ala-Phe-Lys*3-PyrS2-Phe-34ClF-GlnR*3-NH2 |
| I-1337 | Ac-S5-Ala-Asp-Ile-B5-Asp-Asp-Aib-Ala-Phe-Lys*3-PyrS2-Phe-34ClF-GlnR*3-NH2 |
| I-1338 | Ac-S5-Ala-Asp-Ile-B5-Asp-Glu-Aib-Ala-Phe-Lys*3-PyrS2-Phe-34ClF-GlnR*3-NH2 |
| I-1339 | Ac-S5-Ala-Asp-Ile-B5-Asp-Asn-Aib-Ala-Phe-Lys*3-PyrS2-Phe-34ClF-GlnR*3-NH2 |
| I-1340 | Ac-S5-Ala-Asp-Ile-B5-Asp-Gln-Aib-Ala-Phe-Lys*3-PyrS2-Phe-34ClF-GlnR*3-NH2 |
| I-1341 | Ac-S5-Ala-Asp-Ile-B5-Asp-Ser-Aib-Ala-Phe-Lys*3-PyrS2-Phe-34ClF-GlnR*3-NH2 |
| I-1342 | Ac-S5-Tle-Asp-Ile-B5-Asp-3COOHF-Aib-Ala-Phe-Lys*3-PyrS2-Phe-34ClF-GlnR*3-NH2 |
| I-1343 | Ac-S5-Val-Asp-Ile-B5-Asp-3COOHF-Aib-Ala-Phe-Lys*3-PyrS2-Phe-34ClF-GlnR*3-NH2 |
| I-1344 | Ac-S5-Ile-Asp-Ile-B5-Asp-3COOHF-Aib-Ala-Phe-Lys*3-PyrS2-Phe-34ClF-GlnR*3-NH2 |
| I-1345 | Ac-PL3-Asp-Npg-B5-Asp-3COOHF-Aib-Ala-Phe-Lys*3-PyrS2-hCbA-BztA-GlnR*3-Val-Glu-Ala- |
| | NH2 |
| I-1346 | Ac-PL3-Asp-Npg-B5-Asp-3COOHF-Aib-Ala-Phe-Lys*3-PyrS2-hCypA-BztA-GlnR*3-Val-Glu- |
| | Ala-NH2 |
| I-1347 | Ac-PL3-Asp-Npg-B5-Asp-3COOHF-Aib-Ala-Phe-Lys*3-PyrS2-hCha-BztA-GlnR*3-Val-Glu-Ala- |
| | NH2 |
| I-1348 | Ac-PL3-Asp-Npg-B5-Asp-3COOHF-Aib-Ala-Phe-Lys*3-PyrS2-hCbA-34ClF-GlnR*3-Val-Glu- |
| | Ala-NH2 |
| I-1349 | Ac-PL3-Asp-Npg-B5-Asp-3COOHF-Aib-Ala-Phe-Lys*3-PyrS2-hCypA-34ClF-GlnR*3-Val-Glu- |
| | Ala-NH2 |
| I-1350 | Ac-PL3-Asp-Npg-B5-Asp-3COOHF-Aib-Ala-Phe-Lys*3-PyrS2-hCha-34ClF-GlnR*3-Val-Glu-Ala- |
| | NH2 |
| I-1351 | Ac-PL3-Asp-Ile-B5-Asp-3COOHF-Aib-Ala-Phe-Lys*3-PyrS2-Phe-34ClF-GlnR*3-Thr-Glu-Ala- |
| | NH2 |
| I-1352 | Ac-PL3-Asp-Ile-B5-Asp-3COOHF-Aib-Ala-Phe-Lys*3-PyrS2-Phe-34ClF-GlnR*3-Ser-Glu-Ala- |
| | NH2 |
| I-1353 | Ac-PL3-Asp-Ile-B5-Asp-3COOHF-Aib-Ala-Phe-Lys*3-PyrS2-Phe-34ClF-GlnR*3-Thr-Ser-Ala- |
| | NH2 |
| I-1354 | Ac-PL3-Asp-Ile-B5-Asp-3COOHF-Aib-Ala-Phe-Lys*3-PyrS2-Phe-34ClF-GlnR*3-Ser-Ser-Ala- |
| | NH2 |
| I-1355 | Ac-PL3-Asp-Ile-B5-Asp-3COOHF-Aib-Ala-Phe-Lys*3-PyrS2-Phe-34ClF-GlnR*3-Thr-Lys-Ala- |
| | NH2 |
| I-1356 | Ac-PL3-Asp-Ile-B5-Asp-3COOHF-Aib-Ala-Phe-Lys*3-PyrS2-Phe-34ClF-GlnR*3-Ser-Lys-Ala- |
| | NH2 |
| I-1357 | Ac-PL3-Asp-Ile-B5-Asp-3COOHF-Aib-Ala-Phe-Lys*3-PyrS2-Phe-34ClF-GlnR*3-Thr-Glu-Leu- |
| | NH2 |
| I-1358 | Ac-PL3-Asp-Ile-B5-Asp-3COOHF-Aib-Ala-Phe-Lys*3-PyrS2-Phe-34ClF-GlnR*3-Ser-Glu-Leu- |
| | NH2 |
| I-1359 | Ac-PL3-Asp-Ile-B5-Asp-3COOHF-Aib-Ala-Phe-Lys*3-PyrS2-Phe-34ClF-GlnR*3-Thr-Ser-Leu- |
| | NH2 |
| I-1360 | Ac-PL3-Asp-Ile-B5-Asp-3COOHF-Aib-Ala-Phe-Lys*3-PyrS2-Phe-34ClF-GlnR*3-Ser-Ser-Leu- |
| | NH2 |
| I-1361 | Ac-PL3-Asp-Ile-B5-Asp-3COOHF-Aib-Ala-Phe-Lys*3-PyrS2-Phe-34ClF-GlnR*3-Thr-Lys-Leu- |
| | NH2 |
| I-1362 | Ac-PL3-Asp-Ile-B5-Asp-3COOHF-Aib-Ala-Phe-Lys*3-PyrS2-Phe-34ClF-GlnR*3-Ser-Lys-Leu- |
| | NH2 |
| I-1363 | Ac-PL3-Asp-Npg-B5-Asp-3COOHF-Aib-Ala-Phe-Leu-[4Abu]DapAc7-3thi-BztA-Ala-GlnR-NH2 |
| I-1364 | Ac-PL3-Asp-Npg-B5-Asp-3COOHF-Aib-Ala-Phe-Leu-[SBut][CH2CH2NH]Dap-3thi-BztA-Ala- |
| | GlnR-NH2 |
| I-1365 | Ac-PL3-Asp-Npg-B5-Asp-3COOHF-Aib-Ala-Phe-[m5Meb]Cys-PyrS2-3Thi-BztA-Cys-Ala-NH2 |
| I-1366 | Ac-PL3-Asp-Npg-B5-Asp-3COOHF-Aib-Ala-Phe-[m5Meb]hCys-PyrS2-3Thi-BztA-Cys-Ala-NH2 |
| I-1367 | Ac-PL3-Asp-Ile-B5-Asp-3COOHF-Aib-Ala-Phe-GlnR*3-PyrS2-Phe-34ClF-Lys*3-NH2 |
| I-1368 | Ac-PL3-Asp-Ile-B5-Asp-3COOHF-Aib-Ala-Phe-GlnR*3-PyrS2-Phe-34ClF-Lys*3-NH2 |
| I-1369 | Ac-PL3-Asp-Ile-B5-Asp-3COOHF-Aib-Ala-Phe-1MeK*3-PyrS2-Phe-34ClF-GlnR*3-NH2 |
| I-1370 | Ac-PL3-Asp-Ile-B5-Asp-3COOHF-Aib-Ala-Phe-GlnR*3-PyrS2-Phe-34ClF-1MeK*3-NH2 |
| I-1371 | Ac-PL3-Asp-Ile-B5-Asp-3COOHF-Aib-Ala-Phe-Lys*3-PyrS2-Phe-34ClF-hGlnR*3-NH2 |
| I-1372 | Ac-PL3-Asp-Ile-B5-Asp-3COOHF-Aib-Ala-Phe-hGlnR*3-PyrS2-Phe-34ClF-Lys*3-NH2 |
| I-1373 | Ac-PL3-Asp-Ile-B5-Asp-3COOHF-Aib-Ala-Phe-1MeK*3-PyrS2-Phe-34ClF-hGlnR*3-NH2 |
| I-1374 | Ac-PL3-Asp-Ile-B5-Asp-3COOHF-Aib-Ala-Phe-hGlnR*3-PyrS2-Phe-34ClF-1MeK*3-NH2 |
| I-1375 | Ac-PL3-Asp-Ile-B5-Asp-3COOHF-Aib-Ala-Phe-hGlnR*3-PyrS2-Phe-34ClF-1MeK*3-NH2 |
| I-1376 | Ac-PL3-Asp-Ile-B5-Asp-3COOHF-Aib-Ala-Phe-GlnR*3-PyrS2-Phe-34ClF-AsnEDA*3-NH2 |
| I-1377 | Ac-PL3-Asp-Ile-B5-Asp-3COOHF-Aib-Ala-Phe-GlnR*3-PyrS2-Phe-34ClF-AsnEDA*3-NH2 |
| I-1378 | Ac-PL3-Asp-Ile-B5-Asp-3COOHF-Aib-Ala-Phe-GlnR*3-PyrS2-Phe-34ClF-GlnEDA*3-NH2 |
| I-1379 | Ac-PL3-Asp-Ile-B5-Asp-3COOHF-Aib-Ala-Phe-GlnR*3-PyrS2-Phe-34ClF-AsnPpz*3-NH2 |
| I-1380 | Ac-PL3-Asp-Ile-B5-Asp-3COOHF-Aib-Ala-Phe-GlnR*3-PyrS2-Phe-34ClF-GlnPpz*3-NH2 |
| I-1381 | Ac-PL3-Asp-Ile-B5-Asp-3COOHF-Aib-Ala-Phe-GlnR3APyr*3-PyrS2-Phe-34ClF-GlnR*3-NH2 |
| I-1382 | Ac-PL3-Asp-Ile-B5-Asp-3COOHF-Aib-Ala-Phe-GlnR*3-PyrS2-Phe-34ClF-GlnR3APyr*3-NH2 |
| I-1383 | Ac-PL3-Asp-Ile-B5-Asp-3COOHF-Aib-Ala-Phe-GlnS3APyr*3-PyrS2-Phe-34ClF-GlnR*3-NH2 |
| I-1384 | Ac-PL3-Asp-Ile-B5-Asp-3COOHF-Aib-Ala-Phe-GlnS3APyr*3-PyrS2-Phe-34ClF-GlnR*3-NH2 |
| I-1385 | Ac-PL3-Asp-Ile-B5-Asp-3COOHF-Aib-Ala-Phe-GlnR*3-PyrS2-Phe-34ClF-GlnS3APyr*3-NH2 |
| I-1386 | Ac-PL3-Asp-Npg-B5-Asp-3COOHF-Aib-Ala-Phe-[mXyl]Cys-PyrS2-3Thi-BztA-aMeC-Ala-NH2 |
| I-1387 | Ac-PL3-Asp-Npg-B5-Asp-3COOHF-Aib-Ala-Phe-[mPyr]Cys-PyrS2-3Thi-BztA-aMeC-Ala-NH2 |
| I-1388 | Ac-PL3-Asp-Npg-B5-Asp-3COOHF-Aib-Ala-Phe-[C3]Cys-PyrS2-3Thi-BztA-aMeC-Ala-NH2 |
| I-1389 | Ac-PL3-Asp-Npg-B5-Asp-3COOHF-Aib-Ala-Phe-[IsoE]Cys-PyrS2-3Thi-BztA-aMeC-Ala-NH2 |
| I-1390 | Ac-PL3-Asp-Npg-B5-Asp-3COOHF-Aib-Ala-Phe-[m5Meb]Cys-PyrS2-3Thi-BztA-aMeC-Ala-NH2 |
| I-1391 | Ac-PL3-Asp-Npg-B5-Asp-3COOHF-Aib-Ala-Phe-[33Oxe]Cys-PyrS2-3Thi-BztA-aMeC-Ala-NH2 |
| I-1392 | Ac-PL3-Asp-Npg-B5-Asp-3COOHF-Aib-Ala-Phe-Lys*3-PyrS2-3Thi-BztA-GlnR*3-Ala-Ala- |
| | [Ac]Lys-NH2 |
| I-1393 | Ac-PL3-Asp-Ile-B5-Asp-3COOHF-Aib-Ala-Phe-Lys*3-PyrS2-Phe-34ClF-GlnR*3-Ala-Ala- |
| | [Ac]Lys-NH2 |
| I-1394 | Ac-PL3-Asp-Npg-B5-Asp-3COOHF-Aib-Ala-Phe-Lys*3-PyrS2-3Thi-BztA-GlnR*3-Ala-Ala-Ala- |
| | [Ac]Lys-NH2 |
| I-1395 | Ac-PL3-Asp-Ile-B5-Asp-3COOHF-Aib-Ala-Phe-Lys*3-PyrS2-Phe-34ClF-GlnR*3-Ala-Ala-Ala- |
| | [Ac]Lys-NH2 |
| I-1396 | Ac-PL3-Asp-Npg-B5-Asp-[Ac]Dap-Aib-Ala-Phe-Lys*3-PyrS2-3Thi-BztA-GlnR*3-Ala-NH2 |
| I-1397 | Ac-PL3-Asp-Npg-B5-Asp-[CH2CO2H]Acp-Aib-Ala-Phe-Lys*3-PyrS2-3Thi-BztA-GlnR*3-Ala- |
| | NH2 |
| I-1398 | Ac-PL3-Asp-Npg-B5-Asp-[Pfbn]GA-Aib-Ala-Phe-Lys*3-PyrS2-3Thi-BztA-GlnR*3-Ala-NH2 |
| I-1399 | Ac-PL3-Asp-Npg-B5-Asp-[Tfb]GA-Aib-Ala-Phe-Lys*3-PyrS2-3Thi-BztA-GlnR*3-Ala-NH2 |
| I-1400 | Ac-PL3-Asp-Npg-B5-Asp-3COOHF-Aib-Ala-Phe-Lys*3-PyrS2-3Thi-BztA-GlnR*3-Ala-NHMe |
| I-1401 | Ac-PL3-Asp-Npg-B5-Asp-3COOHF-Aib-Ala-Phe-Lys*3-PyrS2-3Thi-BztA-GlnR*3-Ala-NHMe |
| I-1402 | Ac-PL3-Asp-Npg-B5-Asp-3COOHF-Aib-Ala-Phe-Lys*3-PyrS2-3Thi-BztA-GlnR*3-Ala-Ala- |
| | [mPEG4]Lys-NH2 |
| I-1403 | Ac-PL3-Asp-Npg-B5-Asp-3COOHF-Aib-Ala-Phe-Lys*3-PyrS2-3Thi-BztA-GlnR*3-Ala-Ala- |
| | [mPEG8]Lys-NH2 |
| I-1404 | Ac-PL3-Asp-Npg-B5-Asp-3COOHF-Aib-Ala-Phe-Lys*3-PyrS2-3Thi-BztA-GlnR*3-Ala-Ala- |
| | [mPEG16]Lys-NH2 |
| I-1405 | Ac-PL3-Asp-Ile-B5-Asp-3COOHF-Aib-Ala-Phe-Lys*3-PyrS2-Phe-34ClF-GlnR*3-Ala-Ala- |
| | [mPEG4]Lys-NH2 |
| I-1406 | Ac-PL3-Asp-Ile-B5-Asp-3COOHF-Aib-Ala-Phe-Lys*3-PyrS2-Phe-34ClF-GlnR*3-Ala-Ala- |
| | [mPEG8]Lys-NH2 |
| I-1407 | Ac-PL3-Asp-Ile-B5-Asp-3COOHF-Aib-Ala-Phe-Lys*3-PyrS2-Phe-34ClF-GlnR*3-Ala-Ala- |
| | [mPEG16]Lys-NH2 |
| I-1408 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG8]Lys-NH2 |
| I-1409 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG16]Lys-NH2 |
| I-1410 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG37]Lys-NH2 |
| I-1411 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG4]Lys-NH2 |
| I-1412 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG8]Lys-NH2 |
| I-1413 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG16]Lys-NH2 |
| I-1414 | Ac-PL3-Asp-Ile-B5-Asp-3COOHF-Aib-Ala-Phe-GlnR*3-PyrS2-Phe-34ClF-GlnMe2EDA*3-NH2 |
| I-1415 | Ac-PL3-Asp-Ile-B5-Asp-3COOHF-Aib-Ala-Phe-GlnR*3-PyrS2-Phe-34ClF-AsnMe2EDA*3-NH2 |
| I-1416 | Ac-PL3-Asp-Ile-B5-Asp-3COOHF-Aib-Ala-Phe-GlnR*3-PyrS2-Phe-34ClF-AsnMeEDA*3-NH2 |
| I-1417 | Ac-PL3-Asp-Ile-B5-Asp-3COOHF-Aib-Ala-Phe-AsnMeEDA*3-PyrS2-Phe-34ClF-GlnR*3-NH2 |
| I-1418 | Ac-PL3-Asp-Ile-B5-Asp-3COOHF-Aib-Ala-Phe-AsnMeEDA*3-PyrS2-Phe-34ClF-GlnR*3-NH2 |
| I-1419 | Ac-PL3-Asp-Ile-B5-Asp-3COOHF-Aib-Ala-Phe-GlnR*3-PyrS2-Phe-34ClF-AsnR3APyr*3-NH2 |
| I-1420 | Ac-PL3-Asp-Ile-B5-Asp-3COOHF-Aib-Ala-Phe-AsnR3APyr*3-PyrS2-Phe-34ClF-AsnR*3-NH2 |
| I-1421 | Ac-PL3-Asp-Ile-B5-Asp-3COOHF-Aib-Ala-Phe-AsnR3APyr*3-PyrS2-Phe-34ClF-GlnR*3-NH2 |
| I-1422 | Ac-PL3-Asp-Ile-B5-Asp-3COOHF-Aib-Ala-Phe-AsnR*3-PyrS2-Phe-34ClF-AsnS3APyr*3-NH2 |
| I-1423 | Ac-PL3-Asp-Ile-B5-Asp-3COOHF-Aib-Ala-Phe-GlnR*3-PyrS2-Phe-34ClF-AsnS3APyr*3-NH2 |
| I-1424 | Ac-PL3-Asp-Ile-B5-Asp-3COOHF-Aib-Ala-Phe-AsnS3APyr*3-PyrS2-Phe-34ClF-AsnR*3-NH2 |
| I-1425 | Ac-PL3-Asp-Npg-B5-Asp-3COOHF-Aib-Ala-Phe-[mPyr]hCys-PyrS2-3Thi-BztA-aMeC-Ala-NH2 |
| I-1426 | Ac-PL3-Asp-Npg-B5-Asp-3COOHF-Aib-Ala-Phe-[m5Meb]hCys-PyrS2-3Thi-BztA-aMeC-Ala- |
| | NH2 |
| I-1427 | Ac-PL3-Asp-Npg-B5-Asp-3COOHF-Aib-Ala-Phe-[C3]hCys-PyrS2-3Thi-BztA-aMeC-Ala-NH2 |
| I-1428 | Ac-PL3-Asp-Npg-B5-Asp-3COOHF-Aib-Ala-Phe-[Red]hCys-PyrS2-3Thi-BztA-aMeC-Ala-NH2 |
| I-1429 | Ac-PL3-Asp-Npg-B5-Asp-3COOHF-Aib-Ala-Phe-[IsoE]hCys-PyrS2-3Thi-BztA-aMeC-Ala-NH2 |
| I-1430 | Ac-PL3-Asp-Npg-B5-Asp-3COOHF-Aib-Ala-Phe-[13Ac]hCys-PyrS2-3Thi-BztA-aMeC-Ala-NH2 |
| I-1431 | Ac-PL3-Asp-Npg-B5-Asp-[Succinate]Dap-Aib-Ala-Phe-Lys*3-PyrS2-3Thi-BztA-GlnR*3-Ala-NH2 |
| I-1432 | Ac-PL3-Asp-Npg-B5-Asp-[Malonate]Dap-Aib-Ala-Phe-Lys*3-PyrS2-3Thi-BztA-GlnR*3-Ala-NH2 |
| I-1433 | Ac-PL3-Asp-Npg-B5-Asp-[Me2Mal]Dap-Aib-Ala-Phe-Lys*3-PyrS2-3Thi-BztA-GlnR*3-Ala-NH2 |
| I-1434 | Ac-PL3-Asp-Npg-B5-Asp-[SaiPrSuc]Dap-Aib-Ala-Phe-Lys*3-PyrS2-3Thi-BztA-GlnR*3-Ala-NH2 |
| I-1435 | Ac-PL3-Asp-Npg-B5-Asp-[SaMeSuc]Dap-Aib-Ala-Phe-Lys*3-PyrS2-3Thi-BztA-GlnR*3-Ala-NH2 |
| I-1436 | Ac-PL3-Asp-Npg-B5-Asp-[RaiPrSuc]Dap-Aib-Ala-Phe-Lys*3-PyrS2-3Thi-BztA-GlnR*3-Ala-NH2 |
| I-1437 | Ac-PL3-Asp-Npg-B5-Asp-3COOHF-Aib-Ala-Phe-TriAzLys*3-[4VinylBzt]PyrSa-3Thi-BztA- |
| | sAla*3-Ala-NH2 |
| I-1438 | Ac-PL3-Asp-Npg-B5-Asp-3COOHF-Aib-Ala-Phe-TriAzLys*3-[3OHBz]PyrSa-3Thi-BztA-sAla*3- |
| | Ala-NH2 |
| I-1439 | Ac-PL3-Asp-Npg-B5-Asp-3COOHF-Aib-Ala-Phe-TriAzLys*3-[3OHBz]PyrSa-3Thi-BztA-sAla*3- |
| | Ala-NH2 |
| I-1440 | Ac-PL3-Asp-Npg-B5-Asp-3COOHF-Aib-Ala-Phe-TriAzLys*3-[CO][Val]PyrSa-3Thi-BztA- |
| | sAla*3-Ala-NH2 |
| I-1441 | Ac-PL3-Asp-Npg-B5-Asp-3COOHF-Aib-Ala-Phe-TriAzLys*3-[CO][Val]PyrSa-3Thi-BztA- |
| | sAla*3-Ala-NH2 |
| I-1442 | Ac-PL3-Asp-Npg-B5-Asp-3COOHF-Aib-Ala-Phe-TriAzLys*3-[CO][dVal]PyrSa-3Thi-BztA- |
| | sAla*3-Ala-NH2 |
| I-1443 | Ac-PL3-Asp-Npg-B5-Asp-3COOHF-Aib-Ala-Phe-TriAzLys*3-[CO][Sar]PyrSa-3Thi-BztA-sAla*3- |
| | Ala-NH2 |
| I-1444 | Ac-PL3-Asp-Npg-B5-Asp-3COOHF-Aib-Ala-Phe-TriAzLys*3-[CO][Nip]PyrSa-3Thi-BztA- |
| | sAla*3-Ala-NH2 |
| I-1445 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1446 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1447 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1448 | Ac-PL3-Asp-Npg-B5-Asp-3COOHF-Aib-Ala-Phe-TriAzLys*3-[CO][Pro]PyrSa-3Thi-BztA- |
| | sAla*3-Ala-NH2 |
| I-1449 | Ac-PL3-Asp-Npg-B5-Asp-3COOHF-Aib-Ala-Phe-TriAzLys*3-[2Me4VinPhAc2]PyrSa-3Thi-BztA- |
| | sAla*3-Ala-NH2 |
| I-1450 | Ac-PL3-Asp-Npg-B5-Asp-3COOHF-Aib-Ala-Phe-TriAzLys*3-[3SBz]PyrSa-3Thi-BztA-sAla*3- |
| | Ala-NH2 |
| I-1451 | Ac-PL3-Asp-Npg-B5-Asp-3COOHF-Aib-Ala-Phe-[m5Pyr]Cys-PyrS2-3Thi-BztA-Cys-Ala-NH2 |
| I-1452 | Ac-S6-Pro-Asp-Npg-B5-Asp-3COOHF-Aib-Ala-Phe-Lys*3-PyrS2-Phe-34ClF-GlnR*3-NH2 |
| I-1453 | Ac-PL3-Asp-Npg-B5-Asp-3COOHF-Aib-Ala-Phe-[m50Meb]Cys-PyrS2-3Thi-BztA-Cys-Ala-NH2 |
| I-1454 | Ac-PL3-Asp-Npg-B5-Asp-3COOHF-Aib-Ala-Phe-[mPyr]Cys-PyrS2-3Thi-BztA-Pen-Ala-NH2 |
| I-1455 | Ac-PL3-Asp-Npg-B5-Asp-3COOHF-Aib-Ala-Phe-[m5Pyr]Cys-PyrS2-3Thi-BztA-Pen-Ala-NH2 |
| I-1456 | Ac-PL3-Asp-Npg-B5-Asp-3COOHF-Aib-Ala-Phe-[m50Meb]Cys-PyrS2-3Thi-BztA-Pen-Ala-NH2 |
| I-1457 | Ac-PL3-Asp-Npg-B5-Asp-3COOHF-Aib-Ala-Phe-[Red]Cys-PyrS2-3Thi-BztA-Pen-Ala-NH2 |
| I-1458 | Ac-PL3-Asp-Npg-B5-Asp-3COOHF-Aib-Ala-Phe-[IsoE]Cys-PyrS2-3Thi-BztA-Pen-Ala-NH2 |
| I-1459 | Ac-PL3-Asp-Npg-B5-Asp-3COOHF-Aib-Ala-Phe-[C3]Cys-PyrS2-3Thi-BztA-Pen-Ala-NH2 |
| I-1460 | Ac-PL3-Asp-Npg-B5-Asp-3COOHF-Aib-Ala-Phe-[IsoE]hCysOx-PyrS2-3Thi-BztA-aMeC-Ala- |
| | NH2 |
| I-1461 | Ac-PL3-Asp-Npg-B5-Asp-3COOHF-Aib-Ala-Phe-[mPyr]Cys-PyrS2-3Thi-BztA-hCys-Ala-NH2 |
| I-1462 | Ac-PL3-Asp-Npg-B5-Asp-3COOHF-Aib-Ala-Phe-[IsoE]Cys-PyrS2-3Thi-BztA-hCys-Ala-NH2 |
| I-1463 | Ac-S5-Glu-Asp-Npg-B5-Asp-3COOHF-Aib-Ala-Phe-Lys*3-PyrS2-Phe-34ClF-GlnR*3-NH2 |
| I-1464 | Ac-S6-Val-Asp-Npg-B5-Asp-3COOHF-Aib-Ala-Phe-Lys*3-PyrS2-Phe-34ClF-GlnR*3-NH2 |
| I-1465 | Ac-S5-Ala-Asp-Npg-B5-Asp-3COOHF-Aib-Ala-Phe-Lys*3-PyrS2-Phe-34ClF-GlnR*3-NH2 |
| I-1466 | Ac-S5-Val-Asp-Npg-B5-Asp-3COOHF-Aib-Ala-Phe-Lys*3-PyrS2-Phe-34ClF-GlnR*3-NH2 |
| I-1467 | Ac-S6-Glu-Asp-Npg-B5-Asp-3COOHF-Aib-Ala-Phe-Lys*3-PyrS2-Phe-34ClF-GlnR*3-NH2 |
| I-1468 | Ac-S6-Ala-Asp-Npg-B5-Asp-3COOHF-Aib-Ala-Phe-Lys*3-PyrS2-Phe-34ClF-GlnR*3-NH2 |
| I-1469 | Ac-PL3-Asp-Npg-B5-Asp-3COOHF-Aib-Ala-Phe-[mPyr]hCys-PyrS2-3Thi-BztA-Pen-Ala-NH2 |
| I-1470 | Ac-PL3-Asp-Npg-B5-Asp-3COOHF-Aib-Ala-Phe-[m5Pyr]hCys-PyrS2-3Thi-BztA-Pen-Ala-NH2 |
| I-1471 | Ac-PL3-Asp-Npg-B5-Asp-3COOHF-Aib-Ala-Phe-[C3]hCys-PyrS2-3Thi-BztA-Pen-Ala-NH2 |
| I-1472 | Ac-PL3-Asp-Npg-B5-Asp-3COOHF-Aib-Ala-Phe-Lys*3-PyrS2-3Thi-BztA-GlnR*3-Thr-Ser-Ala- |
| | NH2 |
| I-1473 | Ac-PL3-Asp-Npg-B5-Asp-3COOHF-Aib-Ala-Phe-Lys*3-PyrS2-3Thi-BztA-GlnR*3-Thr-Ser-Leu- |
| | NH2 |
| I-1474 | Ac-PL3-Asp-Npg-B5-Asp-3COOHF-Aib-Ala-Phe-Lys*3-PyrS2-3Thi-BztA-GlnR*3-Thr-Ser-Leu- |
| | Pro-NH2 |
| I-1475 | Ac-PL3-Asp-Npg-B5-Asp-3COOHF-Aib-Ala-Phe-Lys*3-PyrS2-3Thi-BztA-GlnR*3-Thr-Ser-Thr- |
| | NH2 |
| I-1476 | Ac-PL3-Asp-Npg-B5-Asp-3COOHF-Aib-Ala-Phe-Lys*3-PyrS2-3Thi-BztA-GlnR*3-Thr-Ser-Thr- |
| | Pro-NH2 |
| I-1477 | Ac-PL3-Asp-Npg-B5-Asp-3COOHF-Aib-Ala-Phe-Lys*3-PyrS2-3Thi-BztA-GlnR*3-Thr-Ser-Pro- |
| | NH2 |
| I-1478 | Ac-PL3-Asp-Npg-B5-Asp-3COOHF-Aib-Ala-Phe-Lys*3-PyrS2-3Thi-BztA-GlnR*3-Val-Ser-Ala- |
| | NH2 |
| I-1479 | Ac-PL3-Asp-Npg-B5-Asp-3COOHF-Aib-Ala-Phe-Lys*3-PyrS2-3Thi-BztA-GlnR*3-Val-Ser-Leu- |
| | NH2 |
| I-1480 | Ac-PL3-Asp-Npg-B5-Asp-3COOHF-Aib-Ala-Phe-Lys*3-PyrS2-3Thi-BztA-GlnR*3-Val-Ser-Leu- |
| | Pro-NH2 |
| I-1481 | Ac-PL3-Asp-Npg-B5-Asp-3COOHF-Aib-Ala-Phe-Lys*3-PyrS2-3Thi-BztA-GlnR*3-Val-Ser-Thr- |
| | NH2 |
| I-1482 | Ac-PL3-Asp-Npg-B5-Asp-3COOHF-Aib-Ala-Phe-Lys*3-PyrS2-3Thi-BztA-GlnR*3-Val-Ser-Thr- |
| | Pro-NH2 |
| I-1483 | Ac-PL3-Asp-Npg-B5-Asp-3COOHF-Aib-Ala-Phe-Lys*3-PyrS2-3Thi-BztA-GlnR*3-Val-Ser-Pro- |
| | NH2 |
| I-1484 | Ac-PL3-Asp-R5-S5-Asp-3COOHF-Aib-Ala-Phe-PyrS2-Lys*3-3Thi-BztA-Ala-GlnR*3-NH2 |
| I-1485 | Ac-PL3-Asp-R5-S5-Asp-3COOHF-Aib-Ala-Phe-PyrS2-Lys*3-3Thi-BztA-Ala-GlnR*3-NH2 |
| I-1486 | NPyroR3-Asp-Npg-B4-Asp-3COOHF-Aib-Ala-Phe-Lys*3-PyrS3-3Thi-BztA-GlnR*3-Ala-NH2 |
| I-1487 | NPyroR3-Asp-Npg-B4-Asp-3COOHF-Aib-Ala-Phe-Lys*3-PyrS3-3Thi-BztA-GlnR*3-Ala-NH2 |
| I-1488 | NPyroR3-Asp-Npg-B5-Asp-3COOHF-Aib-Ala-Phe-Lys*3-PyrS2-3Thi-BztA-GlnR*3-Ala-NH2 |
| I-1489 | NPyroR3-Asp-Npg-B5-Asp-3COOHF-Aib-Ala-Phe-Lys*3-PyrS2-3Thi-BztA-GlnR*3-Ala-NH2 |
| I-1490 | NPyroR3-Asp-Npg-B6-Asp-3COOHF-Aib-Ala-Phe-Lys*3-PyrS1-3Thi-BztA-GlnR*3-Ala-NH2 |
| I-1491 | NPyroR3-Asp-Npg-B6-Asp-3COOHF-Aib-Ala-Phe-Lys*3-PyrS1-3Thi-BztA-GlnR*3-Ala-NH2 |
| I-1492 | NPyroR3-Asp-Ala-B4-Asp-3COOHF-Aib-Ala-Phe-Lys*3-PyrS3-3Thi-BztA-GlnR*3-Ala-NH2 |
| I-1493 | NPyroR3-Asp-Ala-B5-Asp-3COOHF-Aib-Ala-Phe-Lys*3-PyrS2-3Thi-BztA-GlnR*3-Ala-NH2 |
| I-1494 | NPyroR3-Asp-Ala-B6-Asp-3COOHF-Aib-Ala-Phe-Lys*3-PyrS1-3Thi-BztA-GlnR*3-Ala-NH2 |
| I-1495 | NPyroR3-Asp-Ala-B6-Asp-3COOHF-Aib-Ala-Phe-Lys*3-PyrS1-3Thi-BztA-GlnR*3-Ala-NH2 |
| I-1496 | NPyroR3-Asp-Npg-B4-Asp-3COOHF-Aib-Ala-Phe-Lys*3-PyrS3-3Thi-BztA-GlnR*3-Ala-NH2 |
| I-1497 | NPyroR3-Asp-Npg-B5-Asp-3COOHF-Aib-Ala-Phe-Lys*3-PyrS2-3Thi-BztA-GlnR*3-Ala-NH2 |
| I-1498 | NPyroR3-Asp-Npg-B6-Asp-3COOHF-Aib-Ala-Phe-Lys*3-PyrS1-3Thi-BztA-GlnR*3-Ala-NH2 |
| I-1499 | NPyroR3-Asp-Ala-B4-Asp-3COOHF-Aib-Ala-Phe-Lys*3-PyrS3-3Thi-BztA-GlnR*3-Ala-NH2 |
| I-1500 | NPyroR3-Asp-Ala-B5-Asp-3COOHF-Aib-Ala-Phe-Lys*3-PyrS2-3Thi-BztA-GlnR*3-Ala-NH2 |
| I-1501 | NPyroR3-Asp-Ala-B6-Asp-3COOHF-Aib-Ala-Phe-Lys*3-PyrS1-3Thi-BztA-GlnR*3-Ala-NH2 |
| I-1502 | Ac-PL3-Asp-Cha-B5-Asp-3COOHF-Aib-Ala-Phe-Lys*3-PyrS2-2ClF-34ClF-GlnR*3-NH2 |
| I-1503 | Ac-PL3-Asp-Cha-B5-Asp-3COOHF-Ala-Ala-Phe-Lys*3-PyrS2-2ClF-34ClF-GlnR*3-NH2 |
| I-1504 | Ac-PL3-Asp-Cha-B5-Asp-3COOHF-CyLeu-Ala-Phe-Lys*3-PyrS2-2ClF-34ClF-GlnR*3-NH2 |
| I-1505 | Ac-PL3-Asp-Chg-B5-Asp-3COOHF-Aib-Ala-Phe-Lys*3-PyrS2-2ClF-34ClF-GlnR*3-NH2 |
| I-1506 | Ac-PL3-Asp-Chg-B5-Asp-3COOHF-Ala-Ala-Phe-Lys*3-PyrS2-2ClF-34ClF-GlnR*3-NH2 |
| I-1507 | Ac-PL3-Asp-Chg-B5-Asp-3COOHF-CyLeu-Ala-Phe-Lys*3-PyrS2-2ClF-34ClF-GlnR*3-NH2 |
| I-1508 | Ac-PL3-Asp-Ile-B5-Asp-3COOHF-Aib-Ala-Phe-Lys*3-PyrS2-2MeF-34ClF-GlnR*3-NH2 |
| I-1509 | Ac-PL3-Asp-Ile-B5-Asp-3COOHF-Aib-Ala-Phe-Lys*3-PyrS2-2MeF-34ClF-GlnR*3-NH2 |
| I-1510 | Ac-PL3-Asp-Ile-B5-Asp-3COOHF-Ala-Ala-Phe-Lys*3-PyrS2-2MeF-34ClF-GlnR*3-NH2 |
| I-1511 | Ac-PL3-Asp-Ile-B5-Asp-3COOHF-CyLeu-Ala-Phe-Lys*3-PyrS2-2MeF-34ClF-GlnR*3-NH2 |
| I-1512 | Ac-PL3-Asp-Leu-B5-Asp-3COOHF-Aib-Ala-Phe-Lys*3-PyrS2-2MeF-34ClF-GlnR*3-NH2 |
| I-1513 | Ac-PL3-Asp-Leu-B5-Asp-3COOHF-Ala-Ala-Phe-Lys*3-PyrS2-2MeF-34ClF-GlnR*3-NH2 |
| I-1514 | Ac-PL3-Asp-Leu-B5-Asp-3COOHF-CyLeu-Ala-Phe-Lys*3-PyrS2-2MeF-34ClF-GlnR*3-NH2 |
| I-1515 | Ac-PL3-Asp-Cha-B5-Asp-3COOHF-Aib-Ala-Phe-Lys*3-PyrS2-2MeF-34ClF-GlnR*3-NH2 |
| I-1516 | Ac-PL3-Asp-Cha-B5-Asp-3COOHF-Ala-Ala-Phe-Lys*3-PyrS2-2MeF-34ClF-GlnR*3-NH2 |
| I-1517 | Ac-PL3-Asp-Chg-B5-Asp-3COOHF-Aib-Ala-Phe-Lys*3-PyrS2-2MeF-34ClF-GlnR*3-NH2 |
| I-1518 | Ac-PL3-Asp-Chg-B5-Asp-3COOHF-Ala-Ala-Phe-Lys*3-PyrS2-2MeF-34ClF-GlnR*3-NH2 |
| I-1519 | Ac-PL3-Asp-Npg-B5-Asp-3COOHF-Aib-Ala-Phe-[Mxyl]Cys-PyrS2-3Thi-BztA-Cys-Ser-NH2 |
| I-1520 | Ac-PL3-Asp-Npg-B5-Asp-3COOHF-Aib-Ala-Phe-[Mpyr]Cys-PyrS2-3Thi-BztA-Cys-Ser-NH2 |
| I-1521 | Ac-PL3-Asp-Npg-B5-Asp-3COOHF-Aib-Ala-Phe-[Red]Cys-PyrS2-3Thi-BztA-Cys-Ser-NH2 |
| I-1522 | Ac-PL3-Asp-Npg-B5-Asp-3COOHF-Aib-Ala-Phe-[C3]Cys-PyrS2-3Thi-BztA-Cys-Ser-NH2 |
| I-1523 | Ac-S6-Aib-Asp-Npg-B5-Asp-3COOHF-Aib-Ala-Phe-Lys*3-PyrS2-Phe-34ClF-GlnR*3-NH2 |
| I-1524 | Ac-S6-MePro-Asp-Npg-B5-Asp-3COOHF-Aib-Ala-Phe-Lys*3-PyrS2-Phe-34ClF-GlnR*3-NH2 |
| I-1525 | Ac-PL3-Asp-Npg-B5-Asp-3COOHF-Aib-Ala-Phe-Lys*3-PyrS2-3Thi-BztA-GlnR*3-Ala-Ala-Ala- |
| | [PEG4triPEG16]Lys-NH2 |
| I-1526 | Ac-PL3-Asp-Npg-B5-Asp-3COOHF-Aib-Ala-Phe-Lys*3-PyrS2-3Thi-BztA-GlnR*3-Ala-Ala-Ala- |
| | [PEG4triPEG36]Lys-NH2 |
| I-1527 | Ac-PL3-Asp-Cha-B5-Asp-3COOHF-Aib-Ala-Phe-Lys*3-PyrS2-2ClF-BztA-GlnR*3-NH2 |
| I-1528 | Ac-PL3-Asp-Cha-B5-Asp-3COOHF-Ala-Ala-Phe-Lys*3-PyrS2-2ClF-BztA-GlnR*3-NH2 |
| I-1529 | Ac-PL3-Asp-Cha-B5-Asp-3COOHF-CyLeu-Ala-Phe-Lys*3-PyrS2-2ClF-BztA-GlnR*3-NH2 |
| I-1530 | Ac-PL3-Asp-Chg-B5-Asp-3COOHF-Aib-Ala-Phe-Lys*3-PyrS2-2ClF-BztA-GlnR*3-NH2 |
| I-1531 | Ac-PL3-Asp-Chg-B5-Asp-3COOHF-Ala-Ala-Phe-Lys*3-PyrS2-2ClF-BztA-GlnR*3-NH2 |
| I-1532 | Ac-PL3-Asp-Npg-B5-Asp-3COOHF-Aib-Ala-Phe-Lys*3-PyrS2-DipA-34ClF-GlnR*3-NH2 |
| I-1533 | Ac-PL3-Asp-Npg-B5-Asp-3COOHF-Aib-Ala-Phe-Lys*3-PyrS2-DipA-34ClF-GlnR*3-NH2 |
| I-1534 | Ac-PL3-Asp-Ile-B5-Asp-3COOHF-Aib-Ala-Phe-Lys*3-PyrS2-DipA-34ClF-GlnR*3-NH2 |
| I-1535 | Ac-PL3-Asp-Ile-B5-Asp-3COOHF-Aib-Ala-Phe-Lys*3-PyrS2-DipA-34ClF-GlnR*3-NH2 |
| I-1536 | Ac-PL3-Asp-Npg-B5-Asp-3COOHF-Aib-Ala-Phe-Lys*3-PyrS2-DipA-BztA-GlnR*3-NH2 |
| I-1537 | Ac-PL3-Asp-Npg-B5-Asp-3COOHF-Aib-Ala-Phe-Lys*3-PyrS2-2FF-34ClF-GlnR*3-NH2 |
| I-1538 | Ac-PL3-Asp-Npg-B5-Asp-3COOHF-Aib-Ala-Phe-Lys*3-PyrS2-2FF-34ClF-GlnR*3-NH2 |
| I-1539 | Ac-PL3-Asp-Ile-B5-Asp-3COOHF-Aib-Ala-Phe-Lys*3-PyrS2-2FF-34ClF-GlnR*3-NH2 |
| I-1540 | Ac-PL3-Asp-Ile-B5-Asp-3COOHF-Aib-Ala-Phe-Lys*3-PyrS2-2FF-34ClF-GlnR*3-NH2 |
| I-1541 | Ac-PL3-Asp-Npg-B5-Asp-3COOHF-Aib-Ala-Phe-Lys*3-PyrS2-2FF-BztA-GlnR*3-NH2 |
| I-1542 | Ac-PL3-Asp-Npg-B5-Asp-3COOHF-Aib-Ala-Phe-Lys*3-PyrS2-2FF-BztA-GlnR*3-NH2 |
| I-1543 | Ac-PL3-Asp-Ile-B5-Asp-3COOHF-Aib-Ala-Phe-Lys*3-PyrS2-2FF-BztA-GlnR*3-NH2 |
| I-1544 | Ac-PL3-Asp-Ile-B5-Asp-3COOHF-Aib-Ala-Phe-Lys*3-PyrS2-2FF-BztA-GlnR*3-NH2 |
| I-1545 | Ac-PL3-Asp-Npg-B5-Asp-3COOHF-Aib-Ala-Phe-Lys*3-PyrS2-2MeF-BztA-GlnR*3-NH2 |
| I-1546 | Ac-PL3-Asp-Ile-B5-Asp-3COOHF-Aib-Ala-Phe-Lys*3-PyrS2-2MeF-BztA-GlnR*3-NH2 |
| I-1547 | Ac-PL3-Asp-Ile-B5-Asp-3COOHF-Aib-Ala-Phe-Lys*3-PyrS2-2MeF-BztA-GlnR*3-NH2 |
| I-1548 | NPyroR3-Asp-Npg-B5-Asp-3COOHF-Aib-Ala-Phe-Lys*3-PyrS2-3Thi-34ClF-GlnR*3-NH2 |
| I-1549 | NPyroR3-Asp-Ala-B5-Asp-3COOHF-Aib-Ala-Phe-Lys*3-PyrS2-3Thi-34ClF-GlnR*3-NH2 |
| I-1550 | NPyroR3-Asp-Ile-B5-Asp-3COOHF-Aib-Ala-Phe-Lys*3-PyrS2-3Thi-34ClF-GlnR*3-NH2 |
| I-1551 | NPyroR3-Asp-Val-B5-Asp-3COOHF-Aib-Ala-Phe-Lys*3-PyrS2-3Thi-34ClF-GlnR*3-NH2 |
| I-1552 | NPyroR3-Asp-Leu-B5-Asp-3COOHF-Aib-Ala-Phe-Lys*3-PyrS2-3Thi-34ClF-GlnR*3-NH2 |
| I-1553 | NPyroR3-Asp-Phe-B5-Asp-3COOHF-Aib-Ala-Phe-Lys*3-PyrS2-3Thi-34ClF-GlnR*3-NH2 |
| I-1554 | NPyroR3-Asp-Chg-B5-Asp-3COOHF-Aib-Ala-Phe-Lys*3-PyrS2-3Thi-34ClF-GlnR*3-NH2 |
| I-1555 | NPyroR3-Asp-Cha-B5-Asp-3COOHF-Aib-Ala-Phe-Lys*3-PyrS2-3Thi-34ClF-GlnR*3-NH2 |
| I-1556 | NPyroR3-Asp-DipA-B5-Asp-3COOHF-Aib-Ala-Phe-Lys*3-PyrS2-3Thi-34ClF-GlnR*3-NH2 |
| I-1557 | NPyroR3-Asn-Npg-B5-Asp-3COOHF-Aib-Ala-Phe-Lys*3-PyrS2-3Thi-34ClF-GlnR*3-NH2 |
| I-1558 | NPyroR3-Ser-Npg-B5-Asp-3COOHF-Aib-Ala-Phe-Lys*3-PyrS2-3Thi-34ClF-GlnR*3-NH2 |
| I-1559 | NPyroR3-Asp-Npg-B5-Asp-3COOHF-Ala-Ala-Phe-Lys*3-PyrS2-3Thi-34ClF-GlnR*3-NH2 |
| I-1560 | C3a-PL3-Asp-Npg-B5-Asp-3COOHF-Aib-Ala-Phe-Lys*3-PyrS2-Phe-34ClF-GlnR*3-NH2 |
| I-1561 | C3a-PL3-Asp-Npg-B5-Asp-3COOHF-Aib-Ala-Phe-Lys*3-PyrS2-Phe-34ClF-GlnR*3-NH2 |
| I-1562 | Bua-PL3-Asp-Npg-B5-Asp-3COOHF-Aib-Ala-Phe-Lys*3-PyrS2-Phe-34ClF-GlnR*3-NH2 |
| I-1563 | isobutyryl-PL3-Asp-Npg-B5-Asp-3COOHF-Aib-Ala-Phe-Lys*3-PyrS2-Phe-34ClF-GlnR*3-NH2 |
| I-1564 | Cpc-PL3-Asp-Npg-B5-Asp-3COOHF-Aib-Ala-Phe-Lys*3-PyrS2-Phe-34ClF-GlnR*3-NH2 |
| I-1565 | Cpc-PL3-Asp-Npg-B5-Asp-3COOHF-Aib-Ala-Phe-Lys*3-PyrS2-Phe-34ClF-GlnR*3-NH2 |
| I-1566 | Cbc-PL3-Asp-Npg-B5-Asp-3COOHF-Aib-Ala-Phe-Lys*3-PyrS2-Phe-34ClF-GlnR*3-NH2 |
| I-1567 | Cbc-PL3-Asp-Npg-B5-Asp-3COOHF-Aib-Ala-Phe-Lys*3-PyrS2-Phe-34ClF-GlnR*3-NH2 |
| I-1568 | CypCO-PL3-Asp-Npg-B5-Asp-3COOHF-Aib-Ala-Phe-Lys*3-PyrS2-Phe-34ClF-GlnR*3-NH2 |
| I-1569 | CypCO-PL3-Asp-Npg-B5-Asp-3COOHF-Aib-Ala-Phe-Lys*3-PyrS2-Phe-34ClF-GlnR*3-NH2 |
| I-1570 | 4THPCO-PL3-Asp-Npg-B5-Asp-3COOHF-Aib-Ala-Phe-Lys*3-PyrS2-Phe-34ClF-GlnR*3-NH2 |
| I-1571 | 4THPCO-PL3-Asp-Npg-B5-Asp-3COOHF-Aib-Ala-Phe-Lys*3-PyrS2-Phe-34ClF-GlnR*3-NH2 |
| I-1572 | C3a-PL3-Asp-Cha-B5-Asp-3COOHF-Aib-Ala-Phe-Lys*3-PyrS2-3Thi-34ClF-GlnR*3-NH2 |
| I-1573 | C3a-PL3-Asp-Cha-B5-Asp-3COOHF-Aib-Ala-Phe-Lys*3-PyrS2-3Thi-34ClF-GlnR*3-NH2 |
| I-1574 | Bua-PL3-Asp-Cha-B5-Asp-3COOHF-Aib-Ala-Phe-Lys*3-PyrS2-3Thi-34ClF-GlnR*3-NH2 |
| I-1575 | Bua-PL3-Asp-Cha-B5-Asp-3COOHF-Aib-Ala-Phe-Lys*3-PyrS2-3Thi-34ClF-GlnR*3-NH2 |
| I-1576 | isobutyryl-PL3-Asp-Cha-B5-Asp-3COOHF-Aib-Ala-Phe-Lys*3-PyrS2-3Thi-34ClF-GlnR*3-NH2 |
| I-1577 | Cpc-PL3-Asp-Cha-B5-Asp-3COOHF-Aib-Ala-Phe-Lys*3-PyrS2-3Thi-34ClF-GlnR*3-NH2 |
| I-1578 | Cpc-PL3-Asp-Cha-B5-Asp-3COOHF-Aib-Ala-Phe-Lys*3-PyrS2-3Thi-34ClF-GlnR*3-NH2 |
| I-1579 | Cbc-PL3-Asp-Cha-B5-Asp-3COOHF-Aib-Ala-Phe-Lys*3-PyrS2-3Thi-34ClF-GlnR*3-NH2 |
| I-1580 | CypCO-PL3-Asp-Cha-B5-Asp-3COOHF-Aib-Ala-Phe-Lys*3-PyrS2-3Thi-34ClF-GlnR*3-NH2 |
| I-1581 | 4THPCO-PL3-Asp-Cha-B5-Asp-3COOHF-Aib-Ala-Phe-Lys*3-PyrS2-3Thi-34ClF-GlnR*3-NH2 |
| I-1582 | C3a-PL3-Asp-Ile-B5-Asp-3COOHF-Aib-Ala-Phe-Lys*3-PyrS2-3Thi-34ClF-GlnR*3-NH2 |
| I-1583 | C3a-PL3-Asp-Ile-B5-Asp-3COOHF-Aib-Ala-Phe-Lys*3-PyrS2-3Thi-34ClF-GlnR*3-NH2 |
| I-1584 | Bua-PL3-Asp-Ile-B5-Asp-3COOHF-Aib-Ala-Phe-Lys*3-PyrS2-3Thi-34ClF-GlnR*3-NH2 |
| I-1585 | Bua-PL3-Asp-Ile-B5-Asp-3COOHF-Aib-Ala-Phe-Lys*3-PyrS2-3Thi-34ClF-GlnR*3-NH2 |
| I-1586 | Cpc-PL3-Asp-Ile-B5-Asp-3COOHF-Aib-Ala-Phe-Lys*3-PyrS2-3Thi-34ClF-GlnR*3-NH2 |
| I-1587 | Cpc-PL3-Asp-Ile-B5-Asp-3COOHF-Aib-Ala-Phe-Lys*3-PyrS2-3Thi-34ClF-GlnR*3-NH2 |
| I-1588 | Cbc-PL3-Asp-Ile-B5-Asp-3COOHF-Aib-Ala-Phe-Lys*3-PyrS2-3Thi-34ClF-GlnR*3-NH2 |
| I-1589 | Cbc-PL3-Asp-Ile-B5-Asp-3COOHF-Aib-Ala-Phe-Lys*3-PyrS2-3Thi-34ClF-GlnR*3-NH2 |
| I-1590 | CypCO-PL3-Asp-Ile-B5-Asp-3COOHF-Aib-Ala-Phe-Lys*3-PyrS2-3Thi-34ClF-GlnR*3-NH2 |
| I-1591 | CypCO-PL3-Asp-Ile-B5-Asp-3COOHF-Aib-Ala-Phe-Lys*3-PyrS2-3Thi-34ClF-GlnR*3-NH2 |
| I-1592 | 4THPCO-PL3-Asp-Ile-B5-Asp-3COOHF-Aib-Ala-Phe-Lys*3-PyrS2-3Thi-34ClF-GlnR*3-NH2 |
| I-1593 | 4THPCO-PL3-Asp-Ile-B5-Asp-3COOHF-Aib-Ala-Phe-Lys*3-PyrS2-3Thi-34ClF-GlnR*3-NH2 |
| I-1594 | Ac-PL3-Asp-Ile-B5-Asp-3COOHF-Aib-Ala-Phe-Lys*3-PyrS2-Phe-Phe-GlnR*3-NH2 |
| I-1595 | Ac-PL3-Asp-Ile-B5-Asp-3COOHF-Aib-Ala-Phe-Lys*3-PyrS2-Phe-Phe-GlnR*3-NH2 |
| I-1596 | Ac-PL3-Asp-Ile-B5-Asp-3COOHF-Aib-Ala-Phe-Lys*3-PyrS2-Phe-2ClF-GlnR*3-NH2 |
| I-1597 | Ac-PL3-Asp-Ile-B5-Asp-3COOHF-Aib-Ala-Phe-Lys*3-PyrS2-Phe-2ClF-GlnR*3-NH2 |
| I-1598 | Ac-PL3-Asp-Ile-B5-Asp-3COOHF-Aib-Ala-Phe-Lys*3-PyrS2-Phe-3ClF-GlnR*3-NH2 |
| I-1599 | Ac-PL3-Asp-Ile-B5-Asp-3COOHF-Aib-Ala-Phe-Lys*3-PyrS2-Phe-3ClF-GlnR*3-NH2 |
| I-1600 | Ac-PL3-Asp-Ile-B5-Asp-3COOHF-Aib-Ala-Phe-Lys*3-PyrS2-Phe-4ClF-GlnR*3-NH2 |
| I-1601 | Ac-PL3-Asp-Ile-B5-Asp-3COOHF-Aib-Ala-Phe-Lys*3-PyrS2-Phe-3MeF-GlnR*3-NH2 |
| I-1602 | Ac-PL3-Asp-Ile-B5-Asp-3COOHF-Aib-Ala-Phe-Lys*3-PyrS2-Phe-2BrF-GlnR*3-NH2 |
| I-1603 | Ac-PL3-Asp-Ile-B5-Asp-3COOHF-Aib-Ala-Phe-Lys*3-PyrS2-Phe-2BrF-GlnR*3-NH2 |
| I-1604 | Ac-PL3-Asp-Ile-B5-Asp-3COOHF-Aib-Ala-Phe-Lys*3-PyrS2-Phe-3BrF-GlnR*3-NH2 |
| I-1605 | Ac-PL3-Asp-Ile-B5-Asp-3COOHF-Aib-Ala-Phe-Lys*3-PyrS2-Phe-3BrF-GlnR*3-NH2 |
| I-1606 | Ac-PL3-Asp-Ile-B5-Asp-3COOHF-Aib-Ala-Phe-Lys*3-PyrS2-Phe-4BrF-GlnR*3-NH2 |
| I-1607 | Ac-PL3-Asp-Ile-B5-Asp-3COOHF-Aib-Ala-Phe-Lys*3-PyrS2-Phe-4BrF-GlnR*3-NH2 |
| I-1608 | Ac-PL3-Asp-Ile-B5-Asp-3COOHF-Aib-Ala-Phe-Lys*3-PyrS2-Phe-3F3MeF-GlnR*3-NH2 |
| I-1609 | Ac-PL3-Asp-Ile-B5-Asp-3COOHF-Aib-Ala-Phe-Lys*3-PyrS2-Phe-3F3MeF-GlnR*3-NH2 |
| I-1610 | Ac-PL3-Asp-Ile-B5-Asp-3COOHF-Aib-Ala-Phe-Lys*3-PyrS2-Phe-4F3MeF-GlnR*3-NH2 |
| I-1611 | Ac-PL3-Asp-Ile-B5-Asp-3COOHF-Aib-Ala-Phe-Lys*3-PyrS2-Phe-4F3MeF-GlnR*3-NH2 |
| I-1612 | Ac-PL3-Asp-Ile-B5-Asp-3COOHF-Aib-Ala-Phe-Lys*3-PyrS2-3Thi-2BrF-GlnR*3-NH2 |
| I-1613 | Ac-PL3-Asp-Ile-B5-Asp-3COOHF-Aib-Ala-Phe-Lys*3-PyrS2-3Thi-3BrF-GlnR*3-NH2 |
| I-1614 | Ac-PL3-Asp-Ile-B5-Asp-3COOHF-Aib-Ala-Phe-Lys*3-PyrS2-3Thi-3F3MeF-GlnR*3-NH2 |
| I-1615 | Ac-PL3-Asp-Ile-B5-Asp-3COOHF-Aib-Ala-Phe-Lys*3-PyrS2-3Thi-4F3MeF-GlnR*3-NH2 |
| I-1616 | Ac-S5-Ala-Asp-Cha-B5-Asp-3COOHF-Aib-Ala-Phe-Lys*3-PyrS2-Phe-34ClF-GlnR*3-NH2 |
| I-1617 | Ac-S5-Pro-Asp-Cha-B5-Asp-3COOHF-Aib-Ala-Phe-Lys*3-PyrS2-Phe-34ClF-GlnR*3-NH2 |
| I-1618 | Ac-S5-Val-Asp-Cha-B5-Asp-3COOHF-Aib-Ala-Phe-Lys*3-PyrS2-Phe-34ClF-GlnR*3-NH2 |
| I-1619 | Ac-S5-Glu-Asp-Cha-B5-Asp-3COOHF-Aib-Ala-Phe-Lys*3-PyrS2-Phe-34ClF-GlnR*3-NH2 |
| I-1620 | Ac-S5-Ala-Asp-Chg-B5-Asp-3COOHF-Aib-Ala-Phe-Lys*3-PyrS2-Phe-34ClF-GlnR*3-NH2 |
| I-1621 | Ac-S5-Pro-Asp-Chg-B5-Asp-3COOHF-Aib-Ala-Phe-Lys*3-PyrS2-Phe-34ClF-GlnR*3-NH2 |
| I-1622 | Ac-S5-Val-Asp-Chg-B5-Asp-3COOHF-Aib-Ala-Phe-Lys*3-PyrS2-Phe-34ClF-GlnR*3-NH2 |
| I-1623 | Ac-S5-Glu-Asp-Chg-B5-Asp-3COOHF-Aib-Ala-Phe-Lys*3-PyrS2-Phe-34ClF-GlnR*3-NH2 |
| I-1624 | Ac-S6-Ala-Asp-Cha-B5-Asp-3COOHF-Aib-Ala-Phe-Lys*3-PyrS2-Phe-34ClF-GlnR*3-NH2 |
| I-1625 | Ac-S6-Ala-Asp-Chg-B5-Asp-3COOHF-Aib-Ala-Phe-Lys*3-PyrS2-Phe-34ClF-GlnR*3-NH2 |
| I-1626 | Ac-S5-Ala-Asp-Cha-B5-Asp-3COOHF-Aib-Ala-Phe-Lys*3-PyrS1-Phe-34ClF-GlnR*3-NH2 |
| I-1627 | Ac-S5-Ala-Asp-Chg-B5-Asp-3COOHF-Aib-Ala-Phe-Lys*3-PyrS1-Phe-34ClF-GlnR*3-NH2 |
| I-1628 | Ac-PL3-Asp-Npg-B5-Asp-3COOHF-Aib-Ala-Phe-Lys*3-PyrS2-2F3MeF-BztA-GlnR*3-Lys-NH2 |
| I-1629 | Ac-PL3-Asp-Npg-B5-Asp-3COOHF-Aib-Ala-Phe-Lys*3-PyrS2-2F3MeF-BztA-GlnR*3-Lys-NH2 |
| I-1630 | Ac-PL3-Asn-DipA-B5-Asp-3COOHF-CyLeu-Ala-Phe-Lys*3-PyrS2-3Thi-34ClF-GlnR*3-NH2 |
| I-1631 | Ac-PL3-aThr-DipA-B5-Asp-3COOHF-CyLeu-Ala-Phe-Lys*3-PyrS2-3Thi-34ClF-GlnR*3-NH2 |
| I-1632 | Ac-PL3-Asp-DipA-B5-Asn-3COOHF-CyLeu-Ala-Phe-Lys*3-PyrS2-3Thi-34ClF-GlnR*3-NH2 |
| I-1633 | Ac-PL3-Asp-DipA-B5-aThr-3COOHF-CyLeu-Ala-Phe-Lys*3-PyrS2-3Thi-34ClF-GlnR*3-NH2 |
| I-1634 | Ac-PL3-Asp-DipA-B5-Asp-3COOHF-nLeu-Ala-Phe-Lys*3-PyrS2-3Thi-34ClF-GlnR*3-NH2 |
| I-1635 | Ac-PL3-Asp-DipA-B5-Asp-3COOHF-Thr-Ala-Phe-Lys*3-PyrS2-3Thi-34ClF-GlnR*3-NH2 |
| I-1636 | Ac-PL3-Asn-DipA-B5-Asp-3COOHF-nLeu-Ala-Phe-Lys*3-PyrS2-3Thi-34ClF-GlnR*3-NH2 |
| I-1637 | Ac-PL3-aThr-DipA-B5-Asp-3COOHF-nLeu-Ala-Phe-Lys*3-PyrS2-3Thi-34ClF-GlnR*3-NH2 |
| I-1638 | Ac-PL3-Asp-DipA-B5-Asn-3COOHF-nLeu-Ala-Phe-Lys*3-PyrS2-3Thi-34ClF-GlnR*3-NH2 |
| I-1639 | Ac-PL3-Asp-DipA-B5-aThr-3COOHF-nLeu-Ala-Phe-Lys*3-PyrS2-3Thi-34ClF-GlnR*3-NH2 |
| I-1640 | Ac-PL3-Asp-DipA-B5-Asp-3COOHF-CyLeu-Ala-Phe-Lys*3-PyrS2-2F3MeF-34ClF-GlnR*3-NH2 |
| I-1641 | Ac-PL3-Asp-Cha-B5-Asp-3COOHF-Aib-Ala-Phe-Lys*3-PyrS2-2ClF-BztA-GlnR*3-Leu-NH2 |
| I-1642 | Ac-PL3-Asp-Cha-B5-Asp-3COOHF-Aib-Ala-Phe-Lys*3-PyrS2-2ClF-BztA-GlnR*3-Leu-NH2 |
| I-1643 | Ac-PL3-Asp-Cha-B5-Asp-3COOHF-Aib-Ala-Phe-Lys*3-PyrS2-2ClF-BztA-GlnR*3-Ala-NH2 |
| I-1644 | Ac-PL3-Asp-Cha-B5-Asp-3COOHF-Aib-Ala-Phe-Lys*3-PyrS2-2ClF-BztA-GlnR*3-Ala-NH2 |
| I-1645 | Ac-PL3-Asp-Cha-B5-Asp-3COOHF-Aib-Ala-Phe-Lys*3-PyrS2-Phe-BztA-GlnR*3-Leu-NH2 |
| I-1646 | Ac-PL3-Asp-Cha-B5-Asp-3COOHF-Aib-Ala-Phe-Lys*3-PyrS2-Phe-BztA-GlnR*3-Leu-NH2 |
| I-1647 | Ac-PL3-Asp-Cha-B5-Asp-3COOHF-Aib-Ala-Phe-Lys*3-PyrS2-Phe-BztA-GlnR*3-Ala-NH2 |
| I-1648 | Ac-PL3-Asp-Cha-B5-Asp-3COOHF-Aib-Ala-Phe-Lys*3-PyrS2-Phe-BztA-GlnR*3-NH2 |
| I-1649 | Ac-PL3-Asp-Cha-B5-Asp-3COOHF-Aib-Ala-Phe-Lys*3-PyrS2-Phe-BztA-GlnR*3-NH2 |
| I-1650 | Ac-PL3-Asp-Cha-B5-Asp-3COOHF-Aib-Ala-Phe-Lys*3-PyrS2-2FF-BztA-GlnR*3-Leu-NH2 |
| I-1651 | Ac-PL3-Asp-Cha-B5-Asp-3COOHF-Aib-Ala-Phe-Lys*3-PyrS2-2FF-BztA-GlnR*3-Leu-NH2 |
| I-1652 | Ac-PL3-Asp-Cha-B5-Asp-3COOHF-Aib-Ala-Phe-Lys*3-PyrS2-2FF-BztA-GlnR*3-Ala-NH2 |
| I-1653 | Ac-PL3-Asp-Cha-B5-Asp-3COOHF-Aib-Ala-Phe-Lys*3-PyrS2-2FF-BztA-GlnR*3-NH2 |
| I-1654 | Ac-PL3-Asp-Cha-B5-Asp-3COOHF-Aib-Ala-Phe-Lys*3-PyrS2-2F3MeF-BztA-GlnR*3-Leu-NH2 |
| I-1655 | Ac-PL3-Asp-Cha-B5-Asp-3COOHF-Aib-Ala-Phe-Lys*3-PyrS2-2F3MeF-BztA-GlnR*3-Leu-NH2 |
| I-1656 | Ac-PL3-Asp-Cha-B5-Asp-3COOHF-Aib-Ala-Phe-Lys*3-PyrS2-2F3MeF-BztA-GlnR*3-Ala-NH2 |
| I-1657 | Ac-PL3-Asp-Cha-B5-Asp-3COOHF-Aib-Ala-Phe-Lys*3-PyrS2-2F3MeF-BztA-GlnR*3-Ala-NH2 |
| I-1658 | Ac-PL3-Asp-Cha-B5-Asp-3COOHF-Aib-Ala-Phe-Lys*3-PyrS2-2F3MeF-BztA-GlnR*3-NH2 |
| I-1659 | Ac-PL3-Asp-Cha-B5-Asp-3COOHF-Aib-Ala-Phe-Lys*3-PyrS2-2F3MeF-BztA-GlnR*3-NH2 |
| I-1660 | Ac-PL3-Asp-Npg-B5-Asp-3COOHF-Aib-Ala-Phe-Lys*3-PyrS2-[Bnc]2NH2F-BztA-GlnR*3-Ala- |
| | NH2 |
| I-1661 | Ac-PL3-Asp-Npg-B5-Asp-3COOHF-Aib-Ala-Phe-Lys*3-PyrS2-[Bnc]2NH2F-BztA-GlnR*3-Ala- |
| | NH2 |
| I-1662 | Ac-PL3-Asp-Npg-B5-Asp-3COOHF-Aib-Ala-Phe-Lys*3-PyrS2-[Phc]2NH2F-BztA-GlnR*3-Ala- |
| | NH2 |
| I-1663 | Ac-PL3-Asp-Npg-B5-Asp-3COOHF-Aib-Ala-Phe-Lys*3-PyrS2-[Phc]2NH2F-BztA-GlnR*3-Ala- |
| | NH2 |
| I-1664 | Ac-PL3-Asp-Npg-B5-Asp-3COOHF-Aib-Ala-Phe-Lys*3-PyrS2-[BiPh]2NH2F-BztA-GlnR*3-Ala- |
| | NH2 |
| I-1665 | Ac-PL3-Asp-Npg-B5-Asp-3COOHF-Aib-Ala-Phe-Lys*3-PyrS2-[BiPh]2NH2F-BztA-GlnR*3-Ala- |
| | NH2 |
| I-1666 | Ac-PL3-Asp-Npg-B5-Asp-3COOHF-Aib-Ala-Phe-Lys*3-PyrS2-[MePipAc]2NH2F-BztA-GlnR*3- |
| | Ala-NH2 |
| I-1667 | Ac-PL3-Asp-Npg-B5-Asp-3COOHF-Aib-Ala-Phe-Lys+3-PyrS2-[2IAPAc]2NH2F-BztA-GlnR*3- |
| | Ala-NH2 |
| I-1668 | Ac-PL3-Asp-Npg-B5-Asp-3COOHF-Aib-Ala-Phe-Lys*3-PyrS2-[2IAPAc]2NH2F-BztA-GlnR*3- |
| | Ala-NH2 |
| I-1669 | Ac-PL3-Asp-Npg-B5-Asp-3COOHF-Aib-Ala-Phe-Lys*3-PyrS2-[2IAPAc]2NH2F-BztA-GlnR*3- |
| | Ala-NH2 |
| I-1670 | Ac-PL3-Asp-Npg-B5-Asp-3COOHF-Aib-Ala-Phe-Lys+3-PyrS2-[3PyAc]2NH2F-BztA-GlnR*3- |
| | Ala-NH2 |
| I-1671 | Ac-PL3-Asp-Npg-B5-Asp-3COOHF-Aib-Ala-Phe-Lys*3-PyrS2-[3PyAc]2NH2F-BztA-GlnR*3- |
| | Ala-NH2 |
| I-1672 | Ac-PL3-Asp-Npg-B5-Asp-3COOHF-Aib-Ala-Phe-Lys*3-PyrS2-[2PyCypCO]2NH2F-BztA- |
| | GlnR*3-Ala-NH2 |
| I-1673 | Ac-S5-Asp-Asp-Npg-B5-Asp-3COOHF-Aib-Ala-Phe-Lys*3-PyrS2-Phe-34ClF-GlnR*3-NH2 |
| I-1674 | Ac-S5-Asp-Asn-Npg-B5-Asp-3COOHF-Aib-Ala-Phe-Lys*3-PyrS2-Phe-34ClF-GlnR*3-NH2 |
| I-1675 | Ac-S5-Asp-Ser-Npg-B5-Asp-3COOHF-Aib-Ala-Phe-Lys*3-PyrS2-Phe-34ClF-GlnR*3-NH2 |
| I-1676 | Ac-S5-Asp-Asp-Npg-B5-Glu-3COOHF-Aib-Ala-Phe-Lys*3-PyrS2-Phe-34ClF-GlnR*3-NH2 |
| I-1677 | Ac-S5-Glu-Asn-Npg-B5-Asp-3COOHF-Aib-Ala-Phe-Lys*3-PyrS2-Phe-34ClF-GlnR*3-NH2 |
| I-1678 | Ac-S5-Glu-Ser-Npg-B5-Asp-3COOHF-Aib-Ala-Phe-Lys*3-PyrS2-Phe-34ClF-GlnR*3-NH2 |
| I-1679 | Ac-S5-Glu-Asp-Npg-B5-Glu-3COOHF-Aib-Ala-Phe-Lys*3-PyrS2-Phe-34ClF-GlnR*3-NH2 |
| I-1680 | Ac-PL3-Asn-DipA-B5-Asp-3COOHF-nLeu-Ala-Phe-Lys*3-PyrS2-3Thi-BztA-GlnR*3-NH2 |
| I-1681 | Ac-PL3-Ser-DipA-B5-Asp-3COOHF-nLeu-Ala-Phe-Lys*3-PyrS2-3Thi-BztA-GlnR*3-NH2 |
| I-1682 | Ac-PL3-Thr-DipA-B5-Asp-3COOHF-nLeu-Ala-Phe-Lys*3-PyrS2-3Thi-BztA-GlnR*3-NH2 |
| I-1683 | Ac-PL3-Asp-DipA-B5-Ser-3COOHF-nLeu-Ala-Phe-Lys*3-PyrS2-3Thi-BztA-GlnR*3-NH2 |
| I-1684 | Ac-PL3-Asp-DipA-B5-Thr-3COOHF-nLeu-Ala-Phe-Lys*3-PyrS2-3Thi-BztA-GlnR*3-NH2 |
| I-1685 | Ac-PL3-Asp-DipA-B5-aThr-3COOHF-nLeu-Ala-Phe-Lys*3-PyrS2-3Thi-BztA-GlnR*3-NH2 |
| I-1686 | Ac-PL3-Asp-DipA-B5-Hse-3COOHF-nLeu-Ala-Phe-Lys*3-PyrS2-3Thi-BztA-GlnR*3-NH2 |
| I-1687 | Ac-PL3-aThr-DipA-B5-Asp-3COOHF-nLeu-Ala-3Thi-Lys*3-PyrS2-3Thi-BztA-GlnR*3-NH2 |
| I-1688 | Ac-S5-Ala-Asp-DipA-B5-Asp-3COOHF-nLeu-Ala-Phe-Lys*3-PyrS2-Phe-34ClF-GlnR*3-NH2 |
| I-1689 | Ac-S5-Ala-Asp-DipA-B5-Asp-3COOHF-nLeu-Ala-Phe-Lys*3-PyrS1-Phe-34ClF-GlnR*3-NH2 |
| I-1690 | Ac-S6-Ala-Asp-DipA-B5-Asp-3COOHF-nLeu-Ala-Phe-Lys*3-PyrS2-Phe-34ClF-GlnR*3-NH2 |
| I-1691 | Ac-S6-Ala-Asp-DipA-B5-Asp-3COOHF-nLeu-Ala-Phe-Lys*3-PyrS1-Phe-34ClF-GlnR*3-NH2 |
| I-1692 | Ac-S5-Ala-Asp-DipA-B5-Asp-3COOHF-Aib-Ala-Phe-Lys*3-PyrS2-Phe-34ClF-GlnR*3-NH2 |
| I-1693 | Ac-S5-Ala-Asp-DipA-B5-Asp-3COOHF-Aib-Ala-Phe-Lys*3-PyrS1-Phe-34ClF-GlnR*3-NH2 |
| I-1694 | Ac-S5-Pro-Asp-DipA-B5-Asp-3COOHF-Aib-Ala-Phe-Lys*3-PyrS1-Phe-34ClF-GlnR*3-NH2 |
| I-1695 | Ac-S6-Pro-Asp-DipA-B5-Asp-3COOHF-Aib-Ala-Phe-Lys*3-PyrS2-Phe-34ClF-GlnR*3-NH2 |
| I-1696 | Ac-S6-Pro-Asp-DipA-B5-Asp-3COOHF-Aib-Ala-Phe-Lys*3-PyrS1-Phe-34ClF-GlnR*3-NH2 |
| I-1697 | Ac-PL3-Asp-Ile-B5-Asp-3COOHF-Ala-Ala-Phe-Lys*3-PyrS2-Phe-34ClF-GlnR*3-NH2 |
| I-1698 | Ac-PL3-Asp-Ile-B5-Asp-3COOHF-Ala-Ala-Phe-Lys*3-PyrS1-Phe-34ClF-GlnR*3-NH2 |
| I-1699 | Ac-PL3-Asp-Leu-B5-Asp-3COOHF-Ala-Ala-Phe-Lys*3-PyrS2-Phe-34ClF-GlnR*3-NH2 |
| I-1700 | Ac-PL3-Asp-Leu-B5-Asp-3COOHF-Ala-Ala-Phe-Lys*3-PyrS1-Phe-34ClF-GlnR*3-NH2 |
| I-1701 | Ac-PL3-Asp-DipA-B5-Asp-3COOHF-Ala-Ala-Phe-Lys*3-PyrS2-3Thi-34ClF-GlnR*3-[mPEG8]- |
| | Lys-NH2 |
| I-1702 | Ac-PL3-Asp-DipA-B5-Asp-3COOHF-Ala-Ala-Phe-Lys*3-PyrS2-3Thi-34ClF-GlnR*3-[mPEG37]- |
| | Lys-NH2 |
| I-1703 | Ac-PL3-Asp-DipA-B5-Asp-3COOHF-Ala-Ala-Phe-Lys*3-PyrS2-3Thi-34ClF-GlnR*3-Ala-Ala- |
| | [mPEG8]-Lys-NH2 |
| I-1704 | Ac-PL3-Asp-DipA-B5-Asp-3COOHF-Ala-Ala-Phe-Lys*3-PyrS2-3Thi-34ClF-GlnR*3-Ala-Ala- |
| | [mPEG37]-Lys-NH2 |
| I-1705 | Ac-PL3-Asp-DipA-B5-Asp-3COOHF-Ala-Ala-Phe-Lys*3-PyrS2-3Thi-34ClF-GlnR*3-Ala-Ala- |
| | Ala-[mPEG8]-Lys |
| I-1706 | Ac-PL3-Asp-DipA-B5-Asp-3COOHF-Ala-Ala-Phe-Lys*3-PyrS2-3Thi-34ClF-GlnR*3-Ala-Ala- |
| | Ala-[mPEG37]-Lys |
| I-1707 | Ac-PL3-Asp-Npg-B5-Asp-3COOHF-Aib-Ala-Phe-Lys*3-PyrS2-3Thi-34ClF-GlnR*3-Ala-Ala-Ala- |
| | [mPEG8]-Lys |
| I-1708 | Ac-PL3-Asp-Npg-B5-Asp-3COOHF-Aib-Ala-Phe-Lys*3-PyrS2-3Thi-34ClF-GlnR*3-Ala-Ala-Ala- |
| | [mPEG37]-Lys |
| I-1709 | Ac-PL3-Asp-Npg-B5-Asp-3COOHF-Aib-Ala-Phe-Lys*3-PyrS2-3Thi-BztA-GlnR*3-OH |
| I-1710 | Bua-PL3-Asp-DipA-B5-Asp-3COOHF-CyLeu-Ala-Phe-Lys*3-PyrS2-3Thi-34ClF-GlnR*3-NH2 |
| I-1711 | Isovaleryl-PL3-Asp-DipA-B5-Asp-3COOHF-CyLeu-Ala-Phe-Lys*3-PyrS2-3Thi-34ClF-GlnR*3- |
| | NH2 |
| I-1712 | Cpc-PL3-Asp-DipA-B5-Asp-3COOHF-CyLeu-Ala-Phe-Lys*3-PyrS2-3Thi-34ClF-GlnR*3-NH2 |
| I-1713 | Cbc-PL3-Asp-DipA-B5-Asp-3COOHF-CyLeu-Ala-Phe-Lys*3-PyrS2-3Thi-34ClF-GlnR*3-NH2 |
| I-1714 | CypCO-PL3-Asp-DipA-B5-Asp-3COOHF-CyLeu-Ala-Phe-Lys*3-PyrS2-3Thi-34ClF-GlnR*3-NH2 |
| I-1715 | Bnc-PL3-Asp-DipA-B5-Asp-3COOHF-CyLeu-Ala-Phe-Lys*3-PyrS2-3Thi-34ClF-GlnR*3-NH2 |
| I-1716 | CF3CO-PL3-Asp-DipA-B5-Asp-3COOHF-CyLeu-Ala-Phe-Lys*3-PyrS2-3Thi-34ClF-GlnR*3-NH2 |
| I-1717 | 6QuiAc-PL3-Asp-DipA-B5-Asp-3COOHF-CyLeu-Ala-Phe-Lys*3-PyrS2-3Thi-34ClF-GlnR*3-NH2 |
| I-1718 | 124TriAc-PL3-Asp-DipA-B5-Asp-3COOHF-CyLeu-Ala-Phe-Lys*3-PyrS2-3Thi-34ClF-GlnR*3- |
| | NH2 |
| I-1719 | 5PymAc-PL3-Asp-DipA-B5-Asp-3COOHF-CyLeu-Ala-Phe-Lys*3-PyrS2-3Thi-34ClF-GlnR*3- |
| | NH2 |
| I-1720 | 2PyCypCO-PL3-Asp-DipA-B5-Asp-3COOHF-CyLeu-Ala-Phe-Lys*3-PyrS2-3Thi-34ClF-GlnR*3- |
| | NH2 |
| I-1721 | 2PyBu-PL3-Asp-DipA-B5-Asp-3COOHF-CyLeu-Ala-Phe-Lys*3-PyrS2-3Thi-34ClF-GlnR*3-NH2 |
| I-1722 | 2PyzCO-PL3-Asp-DipA-B5-Asp-3COOHF-CyLeu-Ala-Phe-Lys*3-PyrS2-3Thi-34ClF-GlnR*3- |
| | NH2 |
| I-1723 | Bua-PL3-Asn-DipA-B5-Asp-3COOHF-CyLeu-Ala-Phe-Lys*3-PyrS2-3Thi-34ClF-GlnR*3-NH2 |
| I-1724 | Isovalery1-PL3-Asn-DipA-B5-Asp-3COOHF-CyLeu-Ala-Phe-Lys*3-PyrS2-3Thi-34ClF-GlnR*3- |
| | NH2 |
| I-1725 | Cpc-PL3-Asn-DipA-B5-Asp-3COOHF-CyLeu-Ala-Phe-Lys*3-PyrS2-3Thi-34ClF-GlnR*3-NH2 |
| I-1726 | Cbc-PL3-Asn-DipA-B5-Asp-3COOHF-CyLeu-Ala-Phe-Lys*3-PyrS2-3Thi-34ClF-GlnR*3-NH2 |
| I-1727 | CypCO-PL3-Asn-DipA-B5-Asp-3COOHF-CyLeu-Ala-Phe-Lys*3-PyrS2-3Thi-34ClF-GlnR*3-NH2 |
| I-1728 | Bnc-PL3-Asn-DipA-B5-Asp-3COOHF-CyLeu-Ala-Phe-Lys*3-PyrS2-3Thi-34ClF-GlnR*3-NH2 |
| I-1729 | CF3CO-PL3-Asn-DipA-B5-Asp-3COOHF-CyLeu-Ala-Phe-Lys*3-PyrS2-3Thi-34ClF-GlnR*3-NH2 |
| I-1730 | 6QuiAc-PL3-Asn-DipA-B5-Asp-3COOHF-CyLeu-Ala-Phe-Lys*3-PyrS2-3Thi-34ClF-GlnR*3-NH2 |
| I-1731 | 124TriAc-PL3-Asn-DipA-B5-Asp-3COOHF-CyLeu-Ala-Phe-Lys*3-PyrS2-3Thi-34ClF-GlnR*3- |
| | NH2 |
| I-1732 | 2PyCypCO-PL3-Asn-DipA-B5-Asp-3COOHF-CyLeu-Ala-Phe-Lys*3-PyrS2-3Thi-34ClF-GlnR*3- |
| | NH2 |
| I-1733 | 2PyBu-PL3-Asn-DipA-B5-Asp-3COOHF-CyLeu-Ala-Phe-Lys*3-PyrS2-3Thi-34ClF-GlnR*3-NH2 |
| I-1734 | 2PyzCO-PL3-Asn-DipA-B5-Asp-3COOHF-CyLeu-Ala-Phe-Lys*3-PyrS2-3Thi-34ClF-GlnR*3- |
| | NH2 |
| I-1735 | Ac-PL3-Asp-Npg-B5-Asp-3COOHF-Aib-Ala-Phe-Lys*3-PyrS2-2NH2F-BztA-GlnR*3-Ala-NH2 |
| I-1736 | Ac-PL3-Asp-Npg-B5-Asp-3COOHF-Aib-Ala-Phe-Lys*3-PyrS2-2NH2F-BztA-GlnR*3-Ala-NH2 |
| I-1737 | Ac-PL3-Asp-Npg-B5-Asp-3COOHF-Aib-Ala-Phe-Lys*3-PyrS2-[124TriAc]2NH2F-BztA-GlnR*3- |
| | Ala-NH2 |
| I-1738 | Ac-PL3-Asp-Npg-B5-Asp-3COOHF-Aib-Ala-Phe-Lys*3-PyrS2-[124TriPr]2NH2F-BztA-GlnR*3- |
| | Ala-NH2 |
| I-1739 | Ac-PL3-Asp-Npg-B5-Asp-3COOHF-Aib-Ala-Phe-Lys*3-PyrS2-[6QuiAc]2NH2F-BztA-GlnR*3- |
| | Ala-NH2 |
| I-1740 | Ac-PL3-Asp-Npg-B5-Asp-3COOHF-Aib-Ala-Phe-Lys*3-PyrS2-[2PyAc]2NH2F-BztA-GlnR*3- |
| | Ala-NH2 |
| I-1741 | Ac-PL3-Asp-Npg-B5-Asp-3COOHF-Aib-Ala-Phe-Lys*3-PyrS2-[2PyPrpc]2NH2F-BztA-GlnR*3- |
| | Ala-NH2 |
| I-1742 | Ac-PL3-Asp-Npg-B5-Asp-3COOHF-Aib-Ala-Phe-Lys*3-PyrS2-[3PyPrpc]2NH2F-BztA-GlnR*3- |
| | Ala-NH2 |
| I-1743 | Ac-PL3-Asp-Npg-B5-Asp-3COOHF-Aib-Ala-Phe-Lys*3-PyrS2-[4PyPrpc]2NH2F-BztA-GlnR*3- |
| | Ala-NH2 |
| I-1744 | Ac-PL3-Asp-Npg-B5-Asp-3COOHF-Aib-Ala-Phe-Lys*3-PyrS2-[MeOPr]2NH2F-BztA-GlnR*3- |
| | Ala-NH2 |
| I-1745 | Ac-PL3-Asp-Npg-B5-Asp-3COOHF-Aib-Ala-Phe-Lys*3-PyrS2-[PhOPr]2NH2F-BztA-GlnR*3- |
| | Ala-NH2 |
| I-1746 | Ac-PL3-Asp-Npg-B5-Asp-3COOHF-Aib-Ala-Phe-Lys*3-PyrS2-[Me2MeOPr]2NH2F-BztA- |
| | GlnR*3-Ala-NH2 |
| I-1747 | Ac-PL3-Asp-Npg-B5-Asp-3COOHF-Aib-Ala-Phe-Lys*3-PyrS2-[Me2NAc]2NH2F-BztA-GlnR*3- |
| | Ala-NH2 |
| I-1748 | Ac-PL3-Asp-Npg-B5-Asp-3COOHF-Aib-Ala-Phe-Lys*3-PyrS2-[Me2NAc]2NH2F-BztA-GlnR*3- |
| | Ala-NH2 |
| I-1749 | Ac-PL3-Asp-Npg-B5-Asp-3COOHF-Aib-Ala-Phe-Lys*3-PyrS2-[Me2NPr]2NH2F-BztA-GlnR*3- |
| | Ala-NH2 |
| I-1750 | Ac-PL3-Asp-Npg-B5-Asp-3COOHF-Aib-Ala-Phe-Lys*3-PyrS2-[NdiMeButC]2NH2F-BztA- |
| | GlnR*3-Ala-NH2 |
| I-1751 | Ac-PL3-Asp-Npg-B5-Asp-3COOHF-Aib-Ala-Phe-Lys*3-PyrS2-[3IAPAc]2NH2F-BztA-GlnR*3- |
| | Ala-NH2 |
| I-1752 | Ac-PL3-Asp-Npg-B5-Asp-3COOHF-Aib-Ala-Phe-Lys*3-PyrS2-[15PyraPy]2NH2F-BztA-GlnR*3- |
| | Ala-NH2 |
| I-1753 | Ac-PL3-Asp-Npg-B5-Asp-3COOHF-Aib-Ala-Phe-Lys*3-PyrS2-[MorphAc]2NH2F-BztA-GlnR*3- |
| | Ala-NH2 |
| I-1754 | Ac-PL3-Asp-Npg-B5-Asp-3COOHF-Aib-Ala-Phe-Lys*3-PyrS2-[Nic]2NH2F-BztA-GlnR*3-Ala- |
| | NH2 |
| I-1755 | Ac-PL3-Asp-Npg-B5-Asp-3COOHF-Aib-Ala-Phe-Lys*3-PyrS2-[2PyzCO]2NH2F-BztA-GlnR*3- |
| | Ala-NH2 |
| I-1756 | Ac-PL3-Asp-Npg-B5-Asp-3COOHF-Aib-Ala-Phe-Lys*3-PyrS2-[5pymCO]2NH2F-BztA-GlnR*3- |
| | Ala-NH2 |
| I-1757 | Ac-PL3-Asp-Ile-B5-Asp-3COOHF-Aib-Ala-Phe-Lys*3-PyrS2-Phe-34ClF-dGlnR*3-NH2 |
| I-1758 | Ac-PL3-Asp-Ile-B5-Asp-3COOHF-Aib-Ala-Phe-Lys*3-PyrS2-Phe-34ClF-dGlnR*3-NHMe |
| I-1759 | Ac-PL3-Asp-Ile-B5-Asp-3COOHF-Aib-Ala-Phe-Lys*3-PyrS2-Phe-34ClF-dGlnR*3-Ala-NH2 |
| I-1760 | Ac-PL3-Asp-Ile-B5-Asp-3COOHF-Aib-Ala-Phe-1MeK*3-PyrS2-Phe-34ClF-dGlnR*3-NH2 |
| I-1761 | Ac-PL3-Asp-Ile-B5-Asp-3COOHF-Aib-Ala-Phe-1MeK*3-PyrS2-Phe-34ClF-dGlnR*3-NHMe |
| I-1762 | Ac-PL3-Asp-Ile-B5-Asp-3COOHF-Aib-Ala-Phe-1MeK*3-PyrS2-Phe-34ClF-dGlnR*3-Ala-NH2 |
| I-1763 | Ac-PL3-Asp-Npg-B5-Asp-3COOHF-Aib-Ala-Phe-Lys*3-PyrS2-[3FPyr2c]2NH2F-BztA-GlnR*3- |
| | Ala-NH2 |
| I-1764 | Ac-PL3-Asp-Npg-B5-Asp-3COOHF-Aib-Ala-Phe-Lys*3-PyrS2-[4FPyr3c]2NH2F-BztA-GlnR*3- |
| | Ala-NH2 |
| I-1765 | Ac-PL3-Asp-Npg-B5-Asp-3COOHF-Aib-Ala-Phe-Lys*3-PyrS2-[4FPyr3c]2NH2F-BztA-GlnR*3- |
| | Ala-NH2 |
| I-1766 | Ac-PL3-Asp-DipA-B5-Asp-3COOHF-CyLeu-Ala-Phe-Lys*3-PyrS2-2F3MeF-BztA-GlnR*3-NH2 |
| I-1767 | Ac-PL3-Asp-DipA-B5-Asp-3COOHF-CyLeu-Ala-Phe-Lys*3-PyrS2-2F3MeF-34ClF-GlnR*3-Pro- |
| | NH2 |
| I-1768 | Ac-PL3-Asp-DipA-B5-Asp-3COOHF-CyLeu-Ala-Phe-Lys*3-PyrS2-2F3MeF-BztA-GlnR*3-Pro- |
| | NH2 |
| I-1769 | Ac-PL3-Asp-DipA-B5-Asp-3COOHF-CyLeu-Ala-Phe-Lys*3-PyrS2-2F3MeF-34ClF-GlnR*3-Leu- |
| | NH2 |
| I-1770 | Ac-PL3-Asp-DipA-B5-Asp-3COOHF-CyLeu-Ala-Phe-Lys*3-PyrS2-2F3MeF-BztA-GlnR*3-Leu- |
| | NH2 |
| I-1771 | Ac-PL3-Asp-DipA-B5-Asp-3COOHF-CyLeu-Ala-Phe-Lys*3-PyrS2-2F3MeF-34ClF-GlnR*3-Ser- |
| | NH2 |
| I-1772 | Ac-PL3-Asp-DipA-B5-Asp-3COOHF-CyLeu-Ala-Phe-Lys*3-PyrS2-2F3MeF-BztA-GlnR*3-Ser- |
| | NH2 |
| I-1773 | Ac-PL3-Asp-DipA-B5-Asp-3COOHF-CyLeu-Ala-Phe-Lys*3-PyrS2-2F3MeF-34ClF-GlnR*3-Val- |
| | NH2 |
| I-1774 | Ac-PL3-Asp-DipA-B5-Asp-3COOHF-CyLeu-Ala-Phe-Lys*3-PyrS2-2F3MeF-BztA-GlnR*3-Val- |
| | NH2 |
| I-1775 | Ac-PL3-Asp-DipA-B5-Asp-3COOHF-CyLeu-Ala-Phe-Lys*3-PyrS2-2F3MeF-BztA-GlnR*3-Val- |
| | NH2 |
| I-1776 | Ac-PL3-Asp-DipA-B5-Asp-3COOHF-CyLeu-Ala-Phe-Lys*3-PyrS2-2F3MeF-34ClF-GlnR*3-Phe- |
| | NH2 |
| I-1777 | Ac-PL3-Asp-DipA-B5-Asp-3COOHF-CyLeu-Ala-Phe-Lys*3-PyrS2-2F3MeF-BztA-GlnR*3-Phe- |
| | NH2 |
| I-1778 | Ac-PL3-Asp-Ile-B5-Asp-3COOHF-Aib-Ala-Phe-GlnR*3-PyrS2-Phe-34ClF-dLys*3-Ala-NH2 |
| I-1779 | Ac-PL3-Asp-Ile-B5-Asp-3COOHF-Aib-Ala-Phe-Orn*3-PyrS2-Phe-34ClF-dGlnR*3-NH2 |
| I-1780 | Ac-PL3-Asp-Ile-B5-Asp-3COOHF-Aib-Ala-Phe-Orn*3-PyrS2-Phe-34ClF-dGlnR*3-NH2 |
| I-1781 | Ac-PL3-Asp-Ile-B5-Asp-3COOHF-Aib-Ala-Phe-Orn*3-PyrS2-Phe-34ClF-dGlnR*3-NHMe |
| I-1782 | Ac-PL3-Asp-Ile-B5-Asp-3COOHF-Aib-Ala-Phe-Orn*3-PyrS2-Phe-34ClF-dGlnR*3-Ala-NH2 |
| I-1783 | Ac-S6-Val-Asp-DipA-B5-Asp-3COOHF-Aib-Ala-Phe-Lys*3-PyrS1-Phe-34ClF-GlnR*3-NH2 |
| I-1784 | Ac-S5-Glu-Asp-DipA-B5-Asp-3COOHF-Aib-Ala-Phe-Lys*3-PyrS1-Phe-34ClF-GlnR*3-NH2 |
| I-1785 | Ac-S5-Leu-Asp-DipA-B5-Asp-3COOHF-Aib-Ala-Phe-Lys*3-PyrS1-Phe-34ClF-GlnR*3-NH2 |
| I-1786 | Ac-PL3-Asp-Ile-B5-Asp-Me2Gln-Aib-Ala-Phe-Lys*3-PyrS2-Phe-34ClF-GlnR*3-Ala-NH2 |
| I-1787 | Ac-PL3-Asp-DipA-B5-Asp-3COOHF-Ala-Ala-Phe-Lys*3-PyrS2-3Thi-34ClF-GlnR*3-Ala-Ala- |
| | [Ac-dPEG2]-Lys-NH2-NH2 |
| I-1788 | Ac-PL3-Asp-DipA-B5-Asp-3COOHF-Ala-Ala-Phe-Lys*3-PyrS2-3Thi-34ClF-GlnR*3-Ala-Ala- |
| | [Ac-PEG8]-Lys-NH2-NH2 |
| I-1789 | Ac-PL3-Asp-DipA-B5-Asp-3COOHF-Ala-Ala-Phe-Lys*3-PyrS2-3Thi-34ClF-GlnR*3-Ala-Ala- |
| | [Oct-dPEG2]-Lys-NH2-NH2 |
| I-1790 | Ac-PL3-Asp-DipA-B5-Asp-3COOHF-Ala-Ala-Phe-Lys*3-PyrS2-3Thi-34ClF-GlnR*3-Ala-Ala- |
| | [Oct-PEG8]-Lys-NH2-NH2 |
| I-1791 | Ac-PL3-Asp-DipA-B5-Asp-3COOHF-Ala-Ala-Phe-Lys*3-PyrS2-3Thi-34ClF-GlnR*3-Ala-Ala- |
| | [C18-dPEG2]-Lys-NH2-NH2 |
| I-1792 | Ac-PL3-Asp-DipA-B5-Asp-3COOHF-Ala-Ala-Phe-Lys*3-PyrS2-3Thi-34ClF-GlnR*3-Ala-Ala- |
| | [C18-PEG8]-Lys-NH2-NH2 |
| I-1793 | Ac-PL3-Asp-Ile-B5-Asp-3COOHF-GlnPDA*3-Ala-Phe-Leu-PyrS2-Phe-34ClF-GlnR*3-NH2 |
| I-1794 | Ac-PL3-Asp-Ile-B5-Asp-3COOHF-GlnPDA*3-Ala-Phe-Ala-PyrS2-Phe-34ClF-GlnR*3-NH2 |
| I-1795 | Ac-PL3-Asp-Ile-B5-Asp-3COOHF-GlnPDA*3-Ala-Phe-Val-PyrS2-Phe-34ClF-GlnR*3-NH2 |
| I-1796 | Ac-PL3-Asp-Ile-B5-Asp-3COOHF-GlnBDA*3-Ala-Phe-Leu-PyrS2-Phe-34ClF-GlnR*3-NH2 |
| I-1797 | Ac-PL3-Asp-Ile-B5-Asp-3COOHF-GlnBDA*3-Ala-Phe-Ala-PyrS2-Phe-34ClF-GlnR*3-NH2 |
| I-1798 | Ac-PL3-Asp-Ile-B5-Asp-3COOHF-GlnBDA*3-Ala-Phe-Val-PyrS2-Phe-34ClF-GlnR*3-NH2 |
| I-1799 | Ac-PL3-Asp-Ile-B5-Asp-3COOHF-GlnMePDA*3-Ala-Phe-Leu-PyrS2-Phe-34ClF-GlnR*3-NH2 |
| I-1800 | Ac-PL3-Asp-Ile-B5-Asp-3COOHF-GlnMePDA*3-Ala-Phe-Ala-PyrS2-Phe-34ClF-GlnR*3-NH2 |
| I-1801 | Ac-PL3-Asp-Ile-B5-Asp-3COOHF-GlnMePDA*3-Ala-Phe-Val-PyrS2-Phe-34ClF-GlnR*3-NH2 |
| I-1802 | Ac-PL3-Asp-Ile-B5-Asp-3COOHF-GlnR*3-Ala-Phe-Leu-PyrS2-Phe-34ClF-GlnMePDA*3-NH2 |
| I-1803 | Ac-PL3-Asp-Ile-B5-Asp-3COOHF-GlnR*3-Ala-Phe-Ala-PyrS2-Phe-34ClF-GlnMePDA*3-NH2 |
| I-1804 | Ac-PL3-Asp-Ile-B5-Asp-3COOHF-GlnR*3-Ala-Phe-Val-PyrS2-Phe-34ClF-GlnMePDA*3-NH2 |
| I-1805 | Ac-S5-TfeGA-Asp-DipA-B5-Asp-3COOHF-Aib-Ala-Phe-Lys*3-PyrS1-Phe-34ClF-GlnR*3-NH2 |
| I-1806 | Ac-S5-3COOHF-Asp-DipA-B5-Asp-3COOHF-Aib-Ala-Phe-Lys*3-PyrS1-Phe-34ClF-GlnR*3-NH2 |
| I-1807 | Ac-S5-Thr-Asp-DipA-B5-Asp-3COOHF-Aib-Ala-Phe-Lys*3-PyrS1-Phe-34ClF-GlnR*3-NH2 |
| I-1808 | Ac-S5-Phe-Asp-DipA-B5-Asp-3COOHF-Aib-Ala-Phe-Lys*3-PyrS1-Phe-34ClF-GlnR*3-NH2 |
| I-1809 | Ac-PL3-Asp-Npg-B5-Asp-3COOHF-Aib-Ala-Phe-TriAzLys*3-S3MePyrSc7-3Thi-BztA-sAla*3- |
| | Ala-NH2 |
| I-1810 | Ac-PL3-Asp-Npg-B5-Asp-3COOHF-Aib-Ala-Phe-TriAzLys*3-R3MePyrSc7-3Thi-BztA-sAla*3- |
| | Ala-NH2 |
| I-1811 | Ac-PL3-Asp-Ile-B5-Asp-3COOHF-GlnR*3-Ala-Phe-Ala-PyrS2-Phe-34ClF-GlnT4CyMe*3-NH2 |
| I-1812 | Ac-PL3-Asp-Npg-B5-Asp-3COOHF-Aib-Ala-Phe-TriAzLys*3-S3iPrPyrSc7-3Thi-BztA-sAla*3- |
| | Ala-NH2 |
| I-1813 | Ac-PL3-Asp-Npg-B5-Asp-3COOHF-Aib-Ala-Phe-TriAzLys*3-S3iPrPyrSc7-3Thi-BztA-sAla*3- |
| | Ala-NH2 |
| I-1814 | Ac-PL3-Asp-Npg-B5-Asp-3COOHF-Aib-Ala-Phe-TriAzLys*3-R3iPrPyrSc7-3Thi-BztA-sAla*3- |
| | Ala-NH2 |
| I-1815 | Ac-PL3-Asp-Npg-B5-Asp-3COOHF-Aib-Ala-Phe-Lys*3-PyrS2-3Thi-BztA-GlnR*3-Thr-Ala- |
| | [mPEG8]-Lys-NH2 |
| I-1816 | Ac-PL3-Asp-Npg-B5-Asp-3COOHF-Aib-Ala-Phe-Lys*3-PyrS2-3Thi-BztA-GlnR*3-Thr-Ala- |
| | [mPEG8]-Lys-NH2 |
| I-1817 | Ac-PL3-Asp-Npg-B5-Asp-3COOHF-Aib-Ala-Phe-Lys*3-PyrS2-3Thi-BztA-GlnR*3-Val-Glu- |
| | [mPEG8]-Lys-NH2 |
| I-1818 | Ac-PL3-Asp-Npg-B5-Asp-3COOHF-Aib-Ala-Phe-Lys*3-PyrS2-3Thi-BztA-GlnR*3-Val-Thr- |
| | [mPEG8]-Lys-NH2 |
| I-1819 | Ac-PL3-Asp-Npg-B5-Asp-3COOHF-Aib-Ala-Phe-Lys*3-PyrS2-3Thi-BztA-GlnR*3-Val-Thr- |
| | [mPEG8]-Lys-NH2 |
| I-1820 | Ac-PL3-Asp-Npg-B5-Asp-3COOHF-Aib-Ala-Phe-Lys*3-PyrS2-3Thi-BztA-GlnR*3-Leu-Ser- |
| | [mPEG8]-Lys-NH2 |
| I-1821 | Ac-PL3-Asp-Npg-B5-Asp-3COOHF-Aib-Ala-Phe-Lys*3-PyrS2-3Thi-BztA-GlnR*3-Leu-Ser- |
| | [mPEG8]-Lys-NH2 |
| I-1822 | Ac-PL3-Asp-Npg-B5-Asp-3COOHF-Aib-Ala-Phe-Lys*3-PyrS2-3Thi-BztA-GlnR*3-Aib-Ser- |
| | [mPEG8]-Lys-NH2 |
| I-1823 | Ac-PL3-Asp-Npg-B5-Asp-3COOHF-Aib-Ala-Phe-Lys*3-PyrS2-3Thi-BztA-GlnR*3-Aib-Ser- |
| | [mPEG8]-Lys-NH2 |
| I-1824 | Ac-PL3-Asp-Leu-B5-Asp-3COOHF-GlnMeBDA*3-Ala-Phe-Leu-PyrS2-Phe-34ClF-GlnR*3-NH2 |
| I-1825 | Ac-PL3-Asp-Leu-B5-Asp-3COOHF-GlnMeBDA*3-Ala-Phe-Ala-PyrS2-Phe-34ClF-GlnR*3-NH2 |
| I-1826 | Ac-PL3-Asp-Leu-B5-Asp-3COOHF-GlnMeBDA*3-Ala-Phe-Val-PyrS2-Phe-34ClF-GlnR*3-NH2 |
| I-1827 | Ac-PL3-Asp-Leu-B5-Asp-3COOHF-GlnR*3-Ala-Phe-Leu-PyrS2-Phe-34ClF-GlnMeBDA*3-NH2 |
| I-1828 | Ac-PL3-Asp-Leu-B5-Asp-3COOHF-GlnR*3-Ala-Phe-Ala-PyrS2-Phe-34ClF-GlnMeBDA*3-NH2 |
| I-1829 | Ac-PL3-Asp-Leu-B5-Asp-3COOHF-GlnR*3-Ala-Phe-Val-PyrS2-Phe-34ClF-GlnMeBDA*3-NH2 |
| I-1830 | Ac-PL3-Asp-Leu-B5-Asp-3COOHF-GlnR*3-Ala-Phe-Leu-PyrS2-Phe-34ClF-Gln5DA*3-NH2 |
| I-1831 | Ac-PL3-Asp-Leu-B5-Asp-3COOHF-GlnR*3-Ala-Phe-Ala-PyrS2-Phe-34ClF-Gln5DA*3-NH2 |
| I-1832 | Ac-PL3-Asp-Leu-B5-Asp-3COOHF-GlnR*3-Ala-Phe-Val-PyrS2-Phe-34ClF-Gln5DA*3-NH2 |
| I-1833 | Ac-PL3-Asp-Leu-B5-Asp-3COOHF-GlnR*3-Ala-Phe-Leu-PyrS2-Phe-34ClF-Gln6DA*3-NH2 |
| I-1834 | Ac-PL3-Asp-Leu-B5-Asp-3COOHF-GlnR*3-Ala-Phe-Ala-PyrS2-Phe-34ClF-Gln6DA*3-NH2 |
| I-1835 | Ac-PL3-Asp-Leu-B5-Asp-3COOHF-GlnR*3-Ala-Phe-Val-PyrS2-Phe-34ClF-Gln6DA*3-NH2 |
| I-1836 | Ac-PL3-Asp-Leu-B5-Asp-3COOHF-GlnT4CyMe*3-Ala-Phe-Leu-PyrS2-Phe-34ClF-GlnR*3-NH2 |
| I-1837 | Ac-PL3-Asp-Leu-B5-Asp-3COOHF-GlnT4CyMe*3-Ala-Phe-Val-PyrS2-Phe-34ClF-GlnR*3-NH2 |
| I-1838 | Ac-PL3-Asp-Leu-B5-Asp-3COOHF-GlnR*3-Ala-Phe-Leu-PyrS2-Phe-34ClF-GlnT4CyMe*3-NH2 |
| I-1839 | Ac-PL3-Asp-Leu-B5-Asp-3COOHF-GlnR*3-Ala-Phe-Ala-PyrS2-Phe-34ClF-GlnT4CyMe*3-NH2 |
| I-1840 | Ac-PL3-Asp-Leu-B5-Asp-3COOHF-GlnR*3-Ala-Phe-Val-PyrS2-Phe-34ClF-GlnT4CyMe*3-NH2 |
| I-1841 | Ac-PL3-Asp-DipA-B5-Asp-3COOHF-Ala-Ala-Phe-Lys*3-PyrS2-Phe-BztA-GlnR*3-NH2 |
| I-1842 | Ac-PL3-Asp-DipA-B5-Asp-3COOHF-Ala-Ala-Phe-Lys*3-PyrS2-Phe-BztA-GlnR*3-NH2 |
| I-1843 | Ac-PL3-Asp-DipA-B5-Asp-3COOHF-Ala-Ala-Phe-Lys*3-PyrS2-Phe-BztA-GlnR*3-Pro-NH2 |
| I-1844 | Ac-PL3-Asp-DipA-B5-Asp-3COOHF-Ala-Ala-Phe-Lys*3-PyrS2-Phe-BztA-GlnR*3-dPro-NH2 |
| I-1845 | Ac-PL3-Asp-DipA-B5-Asp-3COOHF-Ala-Ala-Phe-Lys*3-PyrS2-Phe-BztA-GlnR*3-dPro-NH2 |
| I-1846 | Ac-PL3-Asp-DipA-B5-Asp-3COOHF-Ala-Ala-Phe-Lys*3-PyrS2-Phe-BztA-GlnR*3-Ser-NH2 |
| I-1847 | Ac-PL3-Asp-DipA-B5-Asp-3COOHF-Ala-Ala-Phe-Lys*3-PyrS2-Phe-BztA-GlnR*3-Val-NH2 |
| I-1848 | Ac-PL3-Asp-DipA-B5-Asp-3COOHF-Ala-Ala-Phe-Lys*3-PyrS2-Phe-BztA-GlnR*3-dVal-NH2 |
| I-1849 | Ac-PL3-Asp-DipA-B5-Asp-3COOHF-Ala-Ala-Phe-Lys*3-PyrS2-Phe-BztA-GlnR*3-dVal-NH2 |
| I-1850 | Ac-PL3-Asp-DipA-B5-Asp-3COOHF-Leu-Ala-Phe-Lys*3-PyrS2-Phe-BztA-GlnR*3-NH2 |
| I-1851 | Ac-PL3-Asp-DipA-B5-Asp-3COOHF-Leu-Ala-Phe-Lys*3-PyrS2-Phe-BztA-GlnR*3-NH2 |
| I-1852 | Ac-PL3-Asp-DipA-B5-Asp-3COOHF-Val-Ala-Phe-Lys*3-PyrS2-Phe-BztA-GlnR*3-NH2 |
| I-1853 | Ac-PL3-Asp-DipA-B5-Asp-3COOHF-Thr-Ala-Phe-Lys*3-PyrS2-Phe-BztA-GlnR*3-NH2 |
| I-1854 | Ac-PL3-Asp-DipA-B5-Asp-3COOHF-Leu-Ala-Phe-Lys*3-PyrS2-Phe-BztA-GlnR*3-dPro-NH2 |
| I-1855 | Ac-PL3-Asp-DipA-B5-Asp-3COOHF-Val-Ala-Phe-Lys*3-PyrS2-Phe-BztA-GlnR*3-dPro-NH2 |
| I-1856 | Ac-PL3-Asp-DipA-B5-Asp-3COOHF-Thr-Ala-Phe-Lys*3-PyrS2-Phe-BztA-GlnR*3-dPro-NH2 |
| I-1857 | Ac-PL3-Asp-Ile-B5-Asp-3COOHF-Aib-Ala-Phe-sAla*3-PyrS2-Phe-34ClF-TriAzLys*3-NH2 |
| I-1858 | Ac-PL3-Asp-Ile-B5-Asp-3COOHF-Aib-Ala-Phe-TriAzLys*3-PyrS2-Phe-34ClF-sCH2S*3-NH2 |
| I-1859 | Ac-PL3-Asp-Ile-B5-Asp-3COOHF-Aib-Ala-Phe-sCH2S*3-PyrS2-Phe-34ClF-TriAzLys*3-NH2 |
| I-1860 | Ac-PL3-Asp-Ile-B5-Asp-3COOHF-Aib-Ala-Phe-TriAzOrn*3-PyrS2-Phe-34ClF-sCH2S*3-NH2 |
| I-1861 | Ac-PL3-Asp-Ile-B5-Asp-3COOHF-Aib-Ala-Phe-TriAzOrn*3-PyrS2-Phe-34ClF-sCH2S*3-NH2 |
| I-1862 | Ac-PL3-Asp-Ile-B5-Asp-3COOHF-Aib-Ala-Phe-sCH2S*3-PyrS2-Phe-34ClF-TriAzOrn*3-NH2 |
| I-1863 | Ac-PL3-Asp-Ile-B5-Asp-3COOHF-Ala-Ala-Phe-TriAzLys*3-PyrS2-Phe-34ClF-sAla*3-NH2 |
| I-1864 | Ac-PL3-Asp-Ile-B5-Asp-3COOHF-Ala-Ala-Phe-sAla*3-PyrS2-Phe-34ClF-TriAzLys*3-NH2 |
| I-1865 | Ac-PL3-Asp-Ile-B5-Asp-3COOHF-Ala-Ala-Phe-TriAzLys*3-PyrS2-Phe-34ClF-sCH2S*3-NH2 |
| I-1866 | Ac-PL3-Asp-Ile-B5-Asp-3COOHF-Ala-Ala-Phe-sCH2S*3-PyrS2-Phe-34ClF-TriAzLys*3-NH2 |
| I-1867 | Ac-PL3-Asp-Ile-B5-Asp-3COOHF-Ala-Ala-Phe-TriAzOrn*3-PyrS2-Phe-34ClF-sCH2S*3-NH2 |
| I-1868 | Ac-PL3-Asp-Ile-B5-Asp-3COOHF-Ala-Ala-Phe-TriAzOrn*3-PyrS2-Phe-34ClF-sCH2S*3-NH2 |
| I-1869 | Ac-PL3-Asp-Ile-B5-Asp-3COOHF-Ala-Ala-Phe-sCH2S*3-PyrS2-Phe-34ClF-TriAzOrn*3-NH2 |
| I-1870 | Ac-PL3-Asp-Ile-B5-Asp-3COOHF-Ala-Ala-Phe-sCH2S*3-PyrS2-Phe-34ClF-TriAzOrn*3-NH2 |
| I-1871 | Ac-S5-Asp-Asp-DipA-B5-Asp-3COOHF-Aib-Ala-Phe-Lys*3-PyrS1-3Thi-BztA-GlnR*3-NH2 |
| I-1872 | Ac-S5-Glu-Asp-DipA-B5-Asp-3COOHF-Aib-Ala-Phe-Lys*3-PyrS1-3Thi-BztA-GlnR*3-NH2 |
| I-1873 | Ac-S5-AcLys-Asp-DipA-B5-Asp-3COOHF-Aib-Ala-Phe-Lys*3-PyrS1-3Thi-BztA-GlnR*3-NH2 |
| I-1874 | Ac-S5-Leu-Asp-DipA-B5-Asp-3COOHF-Aib-Ala-Phe-Lys*3-PyrS1-3Thi-BztA-GlnR*3-NH2 |
| I-1875 | Ac-S5-3COOHF-Asp-DipA-B5-Asp-3COOHF-Aib-Ala-Phe-Lys*3-PyrS1-3Thi-BztA-GlnR*3-NH2 |
| I-1876 | Ac-S5-Ala-Asp-DipA-B5-Asp-3COOHF-Aib-Ala-Phe-Lys*3-PyrS1-3Thi-BztA-GlnR*3-NH2 |
| I-1877 | Ac-S5-Asp-Asp-Val-B5-Asp-3COOHF-Aib-Ala-Phe-Lys*3-PyrS1-3Thi-BztA-GlnR*3-NH2 |
| I-1878 | Ac-S5-Glu-Asp-Val-B5-Asp-3COOHF-Aib-Ala-Phe-Lys*3-PyrS1-3Thi-BztA-GlnR*3-NH2 |
| I-1879 | Ac-S5-AcLys-Asp-Val-B5-Asp-3COOHF-Aib-Ala-Phe-Lys*3-PyrS1-3Thi-BztA-GlnR*3-NH2 |
| I-1880 | Ac-S5-Leu-Asp-Val-B5-Asp-3COOHF-Aib-Ala-Phe-Lys*3-PyrS1-3Thi-BztA-GlnR*3-NH2 |
| I-1881 | Ac-S5-3COOHF-Asp-Val-B5-Asp-3COOHF-Aib-Ala-Phe-Lys*3-PyrS1-3Thi-BztA-GlnR*3-NH2 |
| I-1882 | Ac-S5-Ala-Asp-Val-B5-Asp-3COOHF-Aib-Ala-Phe-Lys*3-PyrS1-3Thi-BztA-GlnR*3-NH2 |
| I-1883 | Ac-Pro-S5-Ala-Asp-Leu-B5-Asp-3COOHF-Aib-Ala-Phe-Lys*3-PyrS1-Phe-34ClF-GlnR*3-NH2 |
| I-1884 | Ac-Pro-S5-Ala-Asp-Leu-B5-Asp-3COOHF-Aib-Ala-Phe-Lys*3-PyrS1-Phe-34ClF-GlnR*3-NH2 |
| I-1885 | Ac-Pro-S5-Glu-Asp-DipA-B5-Asp-3COOHF-Aib-Ala-Phe-Lys*3-PyrS1-Phe-34ClF-GlnR*3-NH2 |
| I-1886 | Ac-Pro-S5-Glu-Asp-Val-B5-Asp-3COOHF-Aib-Ala-Phe-Lys*3-PyrS1-Phe-34ClF-GlnR*3-NH2 |
| I-1887 | Ac-Pro-S5-Glu-Asp-Leu-B5-Asp-3COOHF-Aib-Ala-Phe-Lys*3-PyrS1-Phe-34ClF-GlnR*3-NH2 |
| I-1888 | Ac-Pro-S5-Ala-Asp-DipA-B5-Asp-3COOHF-Aib-Ala-Phe-Lys*3-PyrS1-Phe-34ClF-GlnR*3-NH2 |
| I-1889 | Ac-Pro-S5-Ala-Asp-Val-B5-Asp-3COOHF-Aib-Ala-Phe-Lys*3-PyrS1-Phe-34ClF-GlnR*3-NH2 |
| I-1890 | Ac-Pro-S5-Ala-Asp-DipA-B5-Asp-3COOHF-Aib-Ala-Phe-Lys*3-PyrS1-Phe-34ClF-GlnR*3-NH2 |
| I-1891 | Ac-Pro-S5-Val-Asp-Val-B5-Asp-3COOHF-Aib-Ala-Phe-Lys*3-PyrS1-Phe-34ClF-GlnR*3-NH2 |
| I-1892 | Ac-Pro-S5-Val-Asp-Leu-B5-Asp-3COOHF-Aib-Ala-Phe-Lys*3-PyrS1-Phe-34ClF-GlnR*3-NH2 |
| I-1893 | Ac-Pro-S5-Leu-Asp-DipA-B5-Asp-3COOHF-Aib-Ala-Phe-Lys*3-PyrS1-Phe-34ClF-GlnR*3-NH2 |
| I-1894 | Ac-Pro-S5-Leu-Asp-Val-B5-Asp-3COOHF-Aib-Ala-Phe-Lys*3-PyrS1-Phe-34ClF-GlnR*3-NH2 |
| I-1895 | Ac-Pro-S5-Leu-Asp-Leu-B5-Asp-3COOHF-Aib-Ala-Phe-Lys*3-PyrS1-Phe-34ClF-GlnR*3-NH2 |
| I-1896 | Ac-PL3-Asp-Leu-B5-Asp-3COOHF-GlnC4CyMe*3-Ala-Phe-Leu-PyrS2-Phe-34ClF-GlnR*3-NH2 |
| I-1897 | Ac-PL3-Asp-Leu-B5-Asp-3COOHF-GlnC4CyMe*3-Ala-Phe-Ala-PyrS2-Phe-34ClF-GlnR*3-NH2 |
| I-1898 | Ac-PL3-Asp-Leu-B5-Asp-3COOHF-GlnR*3-Ala-Phe-Leu-PyrS2-Phe-34ClF-GlnC4CyMe*3-NH2 |
| I-1899 | Ac-PL3-Asp-Leu-B5-Asp-3COOHF-GlnR*3-Ala-Phe-Ala-PyrS2-Phe-34ClF-GlnC4CyMe*3-NH2 |
| I-1900 | Ac-PL3-Asp-Leu-B5-Asp-3COOHF-Gln3ACPip*3-Ala-Phe-Ala-PyrS2-Phe-34ClF-GlnR*3-NH2 |
| I-1901 | Ac-PL3-Asp-Leu-B5-Asp-3COOHF-GlnR*3-Ala-Phe-Leu-PyrS2-Phe-34ClF-Gln3ACPip*3-NH2 |
| I-1902 | Ac-PL3-Asp-Leu-B5-Asp-3COOHF-GlnR*3-Ala-Phe-Ala-PyrS2-Phe-34ClF-Gln3ACPip*3-NH2 |
| I-1903 | Ac-PL3-Asp-Leu-B5-Asp-3COOHF-GlnPipAz*3-Ala-Phe-Leu-PyrS2-Phe-34ClF-GlnR*3-NH2 |
| I-1904 | Ac-PL3-Asp-Leu-B5-Asp-3COOHF-GlnPipAz*3-Ala-Phe-Ala-PyrS2-Phe-34ClF-GlnR*3-NH2 |
| I-1905 | Ac-PL3-Asp-Leu-B5-Asp-3COOHF-GlnR*3-Ala-Phe-Leu-PyrS2-Phe-34ClF-GlnPipAz*3-NH2 |
| I-1906 | Ac-PL3-Asp-Leu-B5-Asp-3COOHF-GlnR*3-Ala-Phe-Ala-PyrS2-Phe-34ClF-GlnPipAz*3-NH2 |
| I-1907 | Ac-PL3-Asp-Leu-B5-Asp-3COOHF-Aib-Ala-Phe-Lys*3-PyrS2-Phe-34ClF-GlnR*3-Ala-NH2 |
| I-1908 | Ac-PL3-Asp-Leu-B5-Asp-3COOHF-Aib-Ala-Phe-Lys*3-PyrS2-Trp-34ClF-GlnR*3-Ala-NH2 |
| I-1909 | Ac-PL3-Asp-Leu-B5-Asp-3COOHF-Aib-Ala-Phe-Lys*3-PyrS2-BztA-34ClF-GlnR*3-Ala-NH2 |
| I-1910 | Ac-PL3-Asp-Leu-B5-Asp-3COOHF-Aib-Ala-Phe-Lys*3-PyrS2-5ClW-34ClF-GlnR*3-Ala-NH2 |
| I-1911 | Ac-PL3-Asp-Leu-B5-Asp-3COOHF-Aib-Ala-Phe-Lys*3-PyrS2-6ClW-34ClF-GlnR*3-Ala-NH2 |
| I-1912 | Ac-PL3-Asp-Leu-B5-Asp-3COOHF-Aib-Ala-Phe-Lys*3-PyrS2-Phe-BztA-GlnR*3-Ala-NH2 |
| I-1913 | Ac-PL3-Asp-Leu-B5-Asp-3COOHF-Aib-Ala-Phe-Lys*3-PyrS2-Trp-BztA-GlnR*3-Ala-NH2 |
| I-1914 | Ac-PL3-Asp-Leu-B5-Asp-3COOHF-Aib-Ala-Phe-Lys*3-PyrS2-BztA-BztA-GlnR*3-Ala-NH2 |
| I-1915 | Ac-PL3-Asp-Leu-B5-Asp-3COOHF-Aib-Ala-Phe-Lys*3-PyrS2-5ClW-BztA-GlnR*3-Ala-NH2 |
| I-1916 | Ac-PL3-Asp-Leu-B5-Asp-3COOHF-Aib-Ala-Phe-Lys*3-PyrS2-6ClW-BztA-GlnR*3-Ala-NH2 |
| I-1917 | Ac-PL3-Asp-DipA-B5-Asp-3COOHF-Ala-Ala-Phe-Lys*3-PyrS2-3Thi-34ClF-GlnR*3-Ala-Ala- |
| | [AdamantC-dPEG2]-Lys-NH2 |
| I-1918 | Ac-PL3-Asp-DipA-B5-Asp-3COOHF-Ala-Ala-Phe-Lys*3-PyrS2-3Thi-34ClF-GlnR*3-Ala-Ala- |
| | [AdamantC-PEG8]--Lys-NH2 |
| I-1919 | Ac-PL3-Asp-DipA-B5-Asp-3COOHF-Ala-Ala-Phe-Lys*3-PyrS2-3Thi-34ClF-GlnR*3-Ala-Ala- |
| | [lithocholate-dPEG2]-Lys-NH2 |
| I-1920 | Ac-PL3-Asp-DipA-B5-Asp-3COOHF-Ala-Ala-Phe-Lys*3-PyrS2-3Thi-34ClF-GlnR*3-Ala-Ala- |
| | [lithocholate-PEG8]-Lys-NH2 |
| I-1921 | Ac-PL3-Asp-Leu-B5-Asp-3COOHF-Gln4Pippip*3-Ala-Phe-Leu-PyrS2-Phe-34ClF-GlnR*3-NH2 |
| I-1922 | Ac-PL3-Asp-Leu-B5-Asp-3COOHF-Gln4Pippip*3-Ala-Phe-Ala-PyrS2-Phe-34ClF-GlnR*3-NH2 |
| I-1923 | Ac-PL3-Asp-Leu-B5-Asp-3COOHF-GlnPip4AE*3-Ala-Phe-Ala-PyrS2-Phe-34ClF-GlnR*3-NH2 |
| I-1924 | Ac-PL3-Asp-Leu-B5-Asp-3COOHF-GlnR*3-Ala-Phe-Leu-PyrS2-Phe-34ClF-GlnPip4AE*3-NH2 |
| I-1925 | Ac-PL3-Asp-Leu-B5-Asp-3COOHF-GlnR*3-Ala-Phe-Val-PyrS2-Phe-34ClF-GlnPip4AE*3-NH2 |
| I-1926 | Ac-PL3-Asp-Leu-B5-Asp-3COOHF-GlnR*3-Ala-Phe-Ala-PyrS2-Phe-34ClF-GlnPip4AE*3-NH2 |
| I-1927 | Ac-S6-Glu-Asp-DipA-B5-Asp-3COOHF-Aib-Ala-Phe-Lys*3-PyrS1-Phe-34ClF-GlnR*3-NH2 |
| I-1928 | Ac-S6-Glu-Asp-DipA-B5-Asp-3COOHF-Aib-Ala-Phe-Lys*3-PyrS1-Phe-BztA-GlnR*3-NH2 |
| I-1929 | Ac-S6-Glu-Asp-DipA-B5-Asp-3COOHF-Aib-Ala-Phe-Lys*3-PyrS1-3Thi-BztA-GlnR*3-NH2 |
| I-1930 | Ac-S6-Glu-Asp-DipA-B5-Asp-3COOHF-Leu-Ala-Phe-Lys*3-PyrS1-Phe-34ClF-GlnR*3-NH2 |
| I-1931 | Ac-S6-Glu-Asp-DipA-B5-Asp-3COOHF-Leu-Ala-Phe-Lys*3-PyrS1-Phe-BztA-GlnR*3-NH2 |
| I-1932 | Ac-S6-Glu-Asp-DipA-B5-Asp-3COOHF-Leu-Ala-Phe-Lys*3-PyrS1-3Thi-BztA-GlnR*3-NH2 |
| I-1933 | Ac-S6-Glu-Asp-DipA-B5-Asp-3COOHF-Aib-Ala-Phe-Lys*3-PyrS1-Phe-34ClF-GlnR*3-Val-NH2 |
| I-1934 | Ac-S6-Glu-Asp-DipA-B5-Asp-3COOHF-Aib-Ala-Phe-Lys*3-PyrS1-3Thi-34ClF-GlnR*3-Val-NH2 |
| I-1935 | Ac-S5-Leu-Asp-Val-B5-Asp-3COOHF-Leu-Ala-Phe-Lys*3-PyrS1-3Thi-BztA-GlnR*3-NH2 |
| I-1936 | Ac-S5-Leu-Asp-Chg-B5-Asp-3COOHF-Leu-Ala-Phe-Lys*3-PyrS1-3Thi-BztA-GlnR*3-NH2 |
| I-1937 | Ac-S6-Leu-Asp-Val-B5-Asp-3COOHF-Leu-Ala-Phe-Lys*3-PyrS1-3Thi-BztA-GlnR*3-NH2 |
| I-1938 | Ac-S6-Leu-Asp-Val-B5-Asp-3COOHF-Aib-Ala-Phe-Lys*3-PyrS1-3Thi-BztA-GlnR*3-NH2 |
| I-1939 | Ac-S6-Leu-Asp-DipA-B5-Asp-3COOHF-Aib-Ala-Phe-Lys*3-PyrS1-3Thi-BztA-GlnR*3-NH2 |
| I-1940 | Ac-Pro-S6-Leu-Asp-Val-B5-Asp-3COOHF-Aib-Ala-Phe-Lys*3-PyrS1-3Thi-BztA-GlnR*3-NH2 |
| I-1941 | Ac-Pro-S6-Leu-Asp-Chg-B5-Asp-3COOHF-Aib-Ala-Phe-Lys*3-PyrS1-3Thi-BztA-GlnR*3-NH2 |

For agents described in the Tables, as described previously, in various embodiments N-terminal cap (N-Term) is connected via R₁ to the amino group (R₁) of the first amino acid (AA1). In some embodiments, a N-Term cap may be properly considered as part of AA1. From there, each carboxylate (R₂) of an amino acid is connected to the amino group (R₁) of the subsequent amino acid, until the carboxylate (R₂) of the final amino acid is connected to R₁ of a C-terminal group. For any amino acid that has a branch point (R₃) and a branching monomer is indicated in brackets, R₁ of the monomer in brackets is attached to R₃ of the amino acid. For the amino acid Dap, with two potential branch points (R₃ and R₄), if two branches are indicated, the R₁ of the first branch is connected to R₃, and R₁ of the second branch connected to R₄. For any pair of amino acids that terminate in a *3 designation, the R₃ groups of each of those amino acids are linked to each other. Likewise, for any pair of amino acids that terminate in a **3 designation, the R₃ groups of those amino acids are linked to each other. For any agent that contains a pair of branching amino acids with R₃ groups, and one contains a branching monomer that contains both R₁ and R₂ groups, then R₁ is attached to the branching amino acid adjacent to it in the sequence, and the R₂ group of the branching monomer is attached to R₃ of the amino acid with no branching monomer designated. For example, in various peptides that have one of Cys, hCys, Pen, or aMeC at position 10 and also one of Cys, hCys, Pen, or aMeC at position 14, and a branching group off of the amino acid residue 10, the R₁ of that branching group is tied to the R₃ of the amino acid residue at position 10, while the R₂ of that branching group is tied to the R₃ of the amino acid residue at position 14. For any amino acid which has a branching amino acid containing R₃ and nothing attached to it by the above, then R₃=H. In various embodiments (e.g., agents described in Table E1, Table E2 and Table E3), PyrS2 is tied together with either R4, R5, R6, or one arm of B5, and if PL3 is present, it is typically tied to the other arm of B5. In various embodiments, if a N-terminal group contains an olefin, it is tied to either AA3, or a branching group off of AA3. If a peptide has been reduced as indicated, then olefins have been hydrogenated to —CH₂—CH₂— after olefin metathesis; if it is indicated “C-term only”, then only the C-terminal side staple, e.g., in many cases PyrS2/R5 olefin staple, has been hydrogenated to —CH₂—CH₂—. For peptides which have not been hydrogenated, two possible staple isomers can be generated for each olefin metathesis, leading to 2” potential isomers (four if n=2). For peptides with the same description and different assigned numbers, these are two separable isomers or compositions comprising one or more isomers. In various embodiments, for a peptide comprising an amino acid residue starting with “Dap7” or “DapAc7”, the olefin of that amino acid residue is tied together with one arm of B5 via olefin metathesis, while the R₃ group of that stapling amino acid residue is tied to the R₃ of another amino acid residue, e.g., GlnR*3 residue, elsewhere in the peptide. Special cases: For I-1484 and I-1485, PL3 is stapled to S5, while the R5 residue is stapled to PyrS2.

In some embodiments, it was confirmed that various peptides, e.g., stapled peptides, comprising residues of amino acids described herein can provide higher affinity than reference peptides that comprise a reference amino acid, e.g., a natural amino acid such as Asp or Glu, but are otherwise identical.

### Example 5. Preparation of an Amino Acid for Peptide Synthesis

In some embodiments, the present disclosure provides various compounds. In some embodiments, such compounds are useful for incorporating related amino acids into peptides. In some embodiments, such a compound is compound 2-2

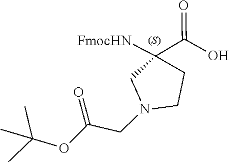

or a salt thereof, whose preparation and uses, including methods, reagents, intermediates, etc., are described in the priority applications, WO 2022/020651 or WO 2022/020652, and are incorporated herein by reference.

### Example 6. Preparation of an Amino Acid for Peptide Synthesis

In some embodiments, the present disclosure provides various compounds. In some embodiments, such compounds are useful for incorporating related amino acids into peptides. In some embodiments, such a compound is

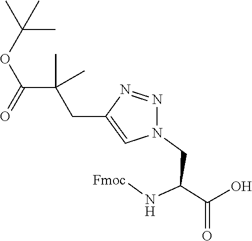

or a salt thereof, whose preparation and uses, including methods, reagents, intermediates, etc., are described in the priority applications, WO 2022/020651 or WO 2022/020652, and are incorporated herein by reference.

### Example 7. Preparation of an Amino Acid for Peptide Synthesis

In some embodiments, the present disclosure provides various compounds. In some embodiments, such compounds are useful for incorporating related amino acids into peptides. In some embodiments, such a compound is

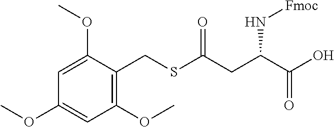

or a salt thereof, whose preparation and uses, including methods, reagents, intermediates, etc., are described in the priority applications, WO 2022/020651 or WO 2022/020652, and are incorporated herein by reference. In some embodiments, the present disclosure provides various compounds. In some embodiments, such a compound is

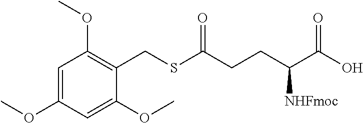

or a salt thereof, whose preparation and uses, including methods, reagents, intermediates, etc., are described in the priority applications, WO 2022/020651 or WO 2022/020652, and are incorporated herein by reference.

### Example 8. Additional Examples of Manufacturing Technologies

Compounds with substitutions on a 2-aminophenylalanine residue (e.g., I-1660 to I-1672) were synthesized in the following manner: Ac-PL3-Asp-Npg-B5-Asp-3COOHF-Aib-Ala-Phe-Lys*3-PyrS2-2NO2F-BztA-GlnR*3-Ala-protide resin was synthesized on a Liberty Blue as above, and the lactam cyclization and olefin metathesis performed as above. The nitro group was reduced by treated with 30 equivalents of tin(II) chloride (2M solution in DMF) at 100° C. for 10 min. The resin was drained and washed with DMF. The resulting peptide was treated with the corresponding carboxylic acid (7 equivalents), HATU (7 equivalents) and diisopropylethylamine (14 equivalents) at 40° C. for 2 h. The coupling reaction was repeated in case of incomplete reaction. The resin was washed with DMF and dichloromethane, and the peptide cleaved and purified as above.

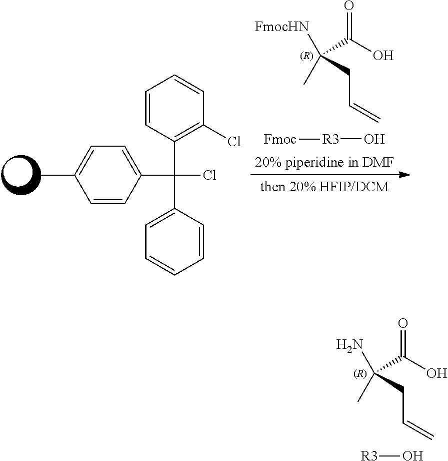

(R)—N-Fmoc-2-(2′-propylenyl)alanine (Fmoc-R3-OH, CAS 288617-76-5) (10.0 g, 30 mmol) was dissolved in dichloromethane (90 mL) and diisopropylethylamine (30.5 mL, 180 mmol) and 2-chlorotrityl resin (28.1 g, 30 mmol) was added. The resin was agitated for 2 h at room temperature, and methanol (30 mL) was added, and the resin agitated for another 30 min. The resin was washed with DMF (3×60 mL), and then treated with 20% piperidine in DMF (60 mL). The resin was agitated for 30 min at room temperature, then the resin washed with DMF (4×60 mL) and methanol (3×60 mL). The resin was then treated with a mixture of hexafluoroisopropanol (18 mL) and dichloromethane (72 mL) and the mixture stirred for 40 min. The resin was filtered off and the resulting solution concentrated to give R3-OH.

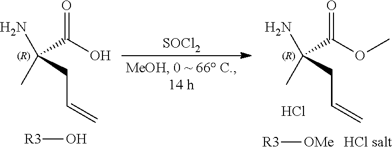

R3-OH (7.88 g, 55.5 mmol) was dissolved in methanol (100 mL) and thionyl chloride (13.2 g, 111 mmol) was added at 0° C., and the reaction warmed to reflux and stirred for 14 h. All volatiles were removed under vacuum to give R3-OMe HCl salt (13.2 g) which was used directly in the next step.

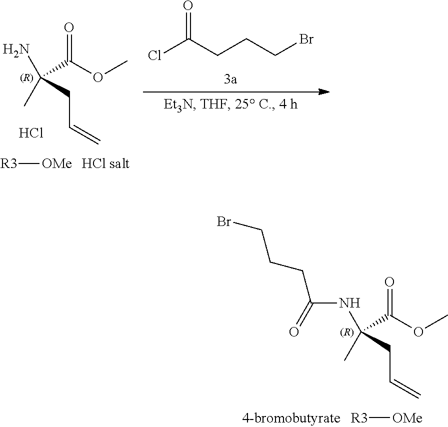

To a solution of R3-OMe HCl salt (6.20 g, 28.6 mmol) in THF (100 mL) and triethylamine (10.0 mmol, 71.7 mmol) was added 4-bromobutyryl chloride (5.0 mL, 43.0 mmol) at room temperature. The reaction was stirred at room temperature for 4 h, then saturated ammonium chloride (100 mL) was added. The mixture was extracted with ethyl acetate (3×100 ml), and the combined organic layers washed with 1M HCl (200 mL), brine (150 mL), and dried with sodium sulfate and concentrated under vacuum. The residue was purified by silica gel chromatography (10% to 50% ethyl acetate in petroleum ether) to give 4-bromobutyrate R3-OMe (3.90 g, 13.3 mmol, 46.5% yield).

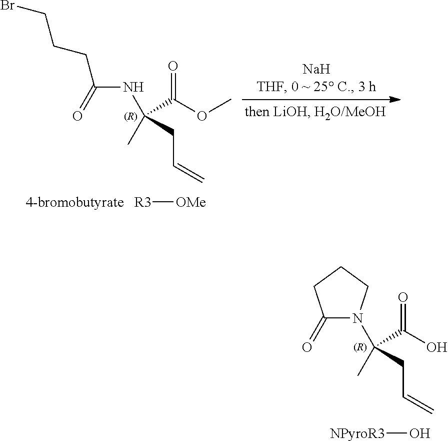

To a solution of 4-bromobutyrate R3-OMe (3.90 g, 13.3 mmol) in THF (70 mL) was added sodium hydride (961 mg, 24 mmol) and the reaction stirred at room temperature for 3 h. The mixture was diluted with ethyl acetate (20 mL) and quenched with saturated ammonium chloride (30 ml). The mixture was extracted with ethyl acetate (3×25 mL), and the combined organic layers dried with sodium sulfate and concentrated. The remaining crude residue was purified by silica gel chromatography (20% to 50% ethyl acetate in petroleum ether) to give a yellow oil. This oil was dissolved in methanol (50 mL) and water (50 ml), and lithium hydroxide hydrate (1.27 g, 30 mmol) was added. The reaction was stirred at room temperature for 1 h. The methanol was removed under vacuum, and the residue extracted with ethyl acetate (30 mL). The aqueous layer was acidified to pH=3 with 1N HCl, and extracted with dichloromethane (5×30 mL). The combined dichloromethane layers were concentrated under vacuum to obtain NPyroR3-OH (2.54 g, 12.8 mmol, 96% yield).

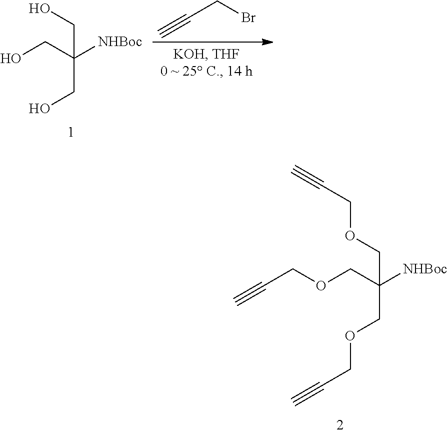

To a solution of compound 1 (25.0 g, 113 mmol) in THF (500 mL) was added potassium hydroxide (38.0 g, 678 mmol) and propargyl bromide (101 g, 678 mmol) in portions. The reaction was stirred at room temperature for 14 h, and the mixture filtered and the filtrate concentrated under vacuum. Silica gel chromatography (1% to 10% ethyl acetate in petroleum ether) yielded compound 2 (23.2 g, 69.2 mmol, 61% yield).

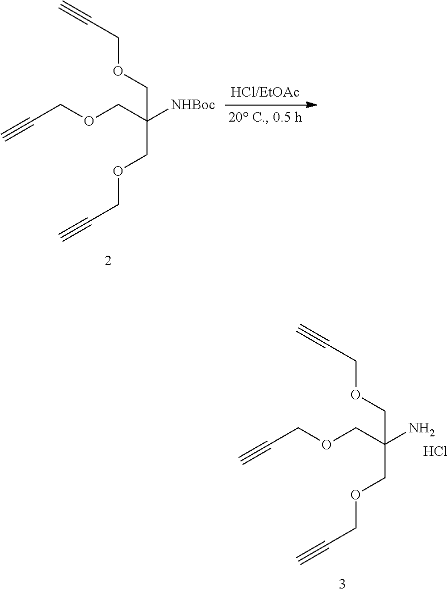 [SMILES: C#CCOCC(COCC#C)(COCC#C)NC(=O)OC(C)(C)C ; C#CCOCC(N)(COCC#C)COCC#C ; Cl]

A mixture of 2 (23.2 g, 69.2 mmol) was stirred in an HCl solution (4 M in ethyl acetate) for 30 min at room temperature. All volatiles were removed under vacuum to give compound 3 (18.4 g, 67.7 mmol, 98% yield) as a light yellow solid.

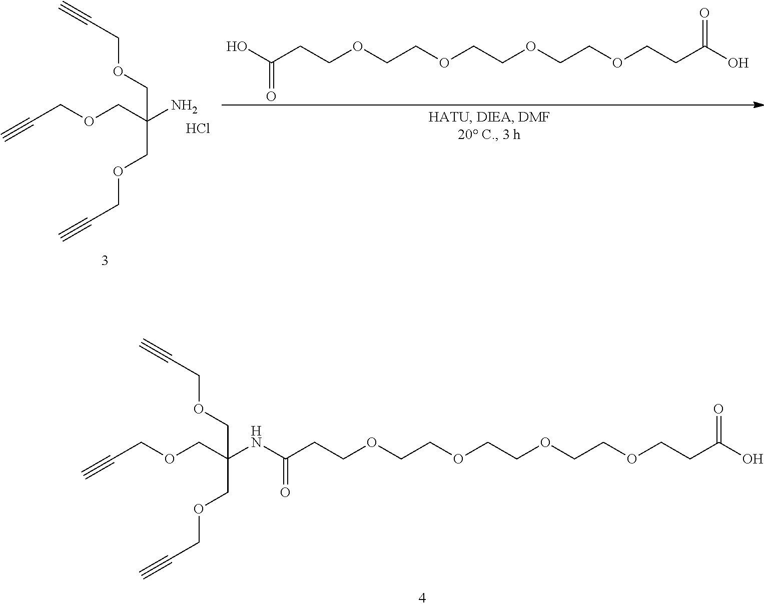 [SMILES: C#CCOCC(N)(COCC#C)COCC#C ; Cl ; O=C(O)CCOCCOCCOCCOCCC(=O)O ; C#CCOCC(COCC#C)(COCC#C)NC(=O)CCOCCOCCOCCOCCC(=O)O]

To a solution of PEG4-diacid (7.74 g, 26.3 mmol) in DMF (100 ml) was added HATU (10.0 g, 26.3 mmol) and diisopropylethylamine (8.33 mL, 47.8 mmol). The mixture was stirred at room temperature for 30 min, then compound 3 (6.5 g, 23.9 mmol) was added. The reaction was stirred at room temperature for 2.5 h, and the reaction diluted with water 9500 mL) and extracted with ethyl acetate (3×200 mL). The combined organic layers were washed with brine (200 mL) and dried with sodium sulfate. The residue was purified by reverse phase HPLC to give compound 4 (3.5 g, 6.84 mmol, 29% yield). LCMS M/Z=512 (M+H).
I-1525, I-1526: Compound with branched PEG at X¹⁸ were synthesized in the following manner: Ac-PL3-Asp-Npg-B5-Asp-3COOHF-Aib-Ala-Phe-Lys*3-PyrS2-3Thi-BztA-GlnR*3-Ala-Ala-Ala-Lys(ivDde)-protide resin was synthesized by solid phase peptide synthesis as above, and the lactam cyclization and olefin metathesis performed as above. The ivDde group was removed by treating the resin with 5% hydrazine in DMF at 40° C. for 30 min, and the resin drained and washed with DMF. The resin was treated with compound 4 (3 equivalents), HATU (3 equivalents), and diisopropylethylamine (10 equivalents) for 3 h at 40° C. The peptide was cleaved as above and purified by reverse phase HPLC. This purified peptide (500 mg) was dissolved in 1:1 acetonitrile: water, and 4 equivalents of either mPEG16-azide (for I-1525) or mPEG36 (for I-1526) was dissolved in 1:1 acetonitrile: water and added to the peptide solution. The pH was adjusted to -8 with ammonium bicarbonate, and copper sulfate (4 equivalents) and sodium ascorbate (5 equivalents) were added, with the pH again adjusted to -8 with ammonium bicarbonate if necessary. The reaction was stirred at 40° C. for 2h, and the final peptide purified by preparative HPLC to give either I-1525 (65% yield) or I-1526 (55% yield).

### Example 9. Provided Technologies can Provide High Selectivity

Among other things, the present disclosure provides various technologies for preparing stapled peptides, including those comprising multiple staples. As described herein, in some embodiments, two or more staples are formed in one step. For example, in some embodiments, two or more staples are formed in a metathesis reaction. In some embodiments, all staples formed by metathesis are formed in a metathesis reaction. In some embodiments, each of such staples are formed through olefin metathesis of terminal olefins. In some embodiments, multiple staples are formed after full lengths of peptides have been achieved. In some embodiments, one or more staples comprising double bonds are formed after full lengths of peptides have been achieved. In some embodiments, all staples comprising double bonds are formed after full lengths of peptides have been achieved. In some embodiments, one or more staples formed through metathesis are formed after full lengths of peptides have been achieved. In some embodiments, all staples formed through metathesis are formed after full lengths of peptides have been achieved.

For example, in some embodiments, to prepare I-66 and I-67, a full length peptide (in some embodiments, prepared on solid phase as shown below) was subject to olefin metathesis:

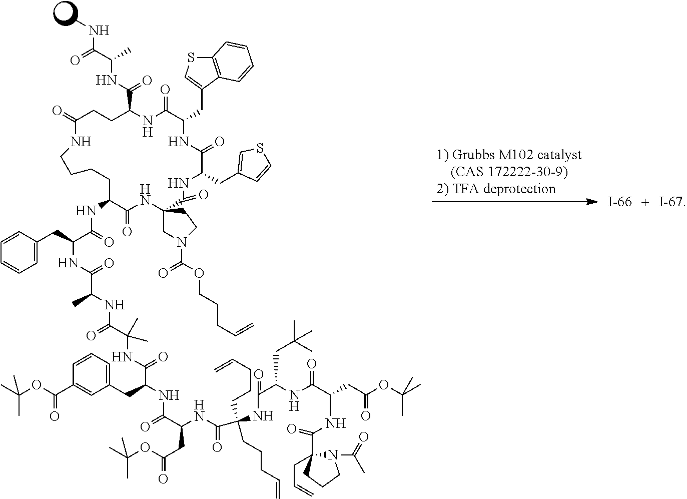

In some embodiments, about 3:1 ratio (I-66:I-66) was observed.

In some embodiments, staples are formed in two or more staples. In some embodiments, two or more staples comprising olefin are formed in two or more staples. In some embodiments, two or more staples are formed in two or more metathesis steps. In some embodiments, two or more metathesis steps utilize different conditions, e.g., different catalysts. In some embodiments, each staple is formed in a separate step. In some embodiments, each staple comprising a double bond is formed in a separate step. In some embodiments, each staple comprising an olefin is formed in a separate step. In some embodiments, each staple formed by olefin metathesis is formed in a separate metathesis step. In some embodiments, stepwise stapling provides improved levels of selectivity to form a desired product (e.g., I-66) over other compounds, e.g., stereoisomers (e.g., for I-66, I-67). For example, in some embodiments, I-66 was prepared as described below, and over 10:1 I-66:I-67 ratio was observed. In some embodiments, the present disclosure provides a composition comprising I-66, wherein the ratio of I-66 to I-67 is about or at least about 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the present disclosure provides a composition comprising I-66 and I-67, wherein the ratio of I-66 to I-67 is about or at least about 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1. In some embodiments, I-66 is provided in a salt form, e.g., a pharmaceutically acceptable salt form. In some embodiments, I-66 is provided in multiple forms including multiple salt forms. In some embodiments, I-67 is provided in a salt form, e.g., a pharmaceutically acceptable salt form. In some embodiments, I-67 is provided in multiple forms including multiple salt forms.

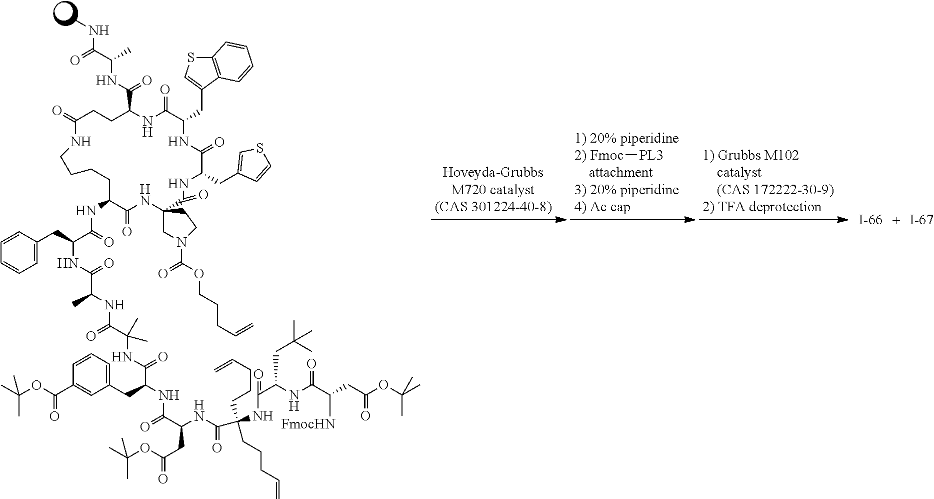

In a preparation, I-66 was synthesized by manual SPPS on Rink amide MBHA resin (98 g, 0.51 mmol/g loading, 50 mmol total). Deprotection steps were performed by treating the resin with 20% piperidine in DMF (v/v, 1000 mL) for thirty minutes with agitation via nitrogen bubbling. The resin was drained and washed with DMF four times. An amino acid to be coupled was dissolved in DMF (800 mL), and the coupling agent indicated below and either diisopropylethylamine (DIEA), or HOAt, were added in the equivalents listed below. Coupling proceeded for 30 minutes at room temperature with nitrogen bubbling, and the amino acid solution drained and the resin washed with DMF four times.

| Amino acid used | Coupling agent/base and amount used |
|---|---|
| Fmoc-Ala-OH (100 mmol) | HBTU (74 mmol), DIEA (75 mmol) |
| Fmoc-Glu(OAllyl)-OH (75 | DIC (75 mmol) and HOAt (75 mmol) |
| mmol) | |
| Fmoc-BztA-OH (65 mmol) | HBTU (61.5 mmol) and DIEA (65 mmol) |
| Fmoc-3Thi-OH (65 mmol) | HBTU (65 mmol) and DIEA (65 mmol) |
| Fmoc-PyrS2-OH (75 mmol) | HBTU (74 mmol), DIEA (75 mmol) |
| Fmoc-Lys(Alloc)-OH (100 | HATU (95 mmol) and DIEA (100 mmol) |
| mmol) | |
| Fmoc-Phe-OH (75 mmol) | HBTU (75 mmol), DIEA (75 mmol) |
| Fmoc-Ala-OH (90 mmol) | HBTU (85 mmol) and DIEA (90 mmol) |
| Fmoc-Aib-OH (100 mmol) | HBTU (95 mmol) and DIEA (100 mmol) |

After Aib addition, prior to Fmoc deprotection, the resin was washed with DMF five times, and dichloromethane five times. A solution of phenylsilane (54 g, 500 mmol) and tetrakis(triphenylphosphine)palladium (O) (5.77 g, 5 mmol) in dichloromethane (500 mL) was added. The reaction proceeded at room temperature for 15 minutes with nitrogen bubbling, and the palladium solution drained. The palladium/phenylsilane treatment was repeated another two times, then the resin drained and washed with DMF five times. The lactam was closed by treating the resin with HOAt (400 mmol) and DIC (400 mmol) in DMF (1000 mL), at room temperature with nitrogen bubbling for 2 h. The resin was drained and washed with DMF four times. The cycles of Fmoc deprotection and amino acid addition continued as above. A repeat coupling step was performed for Fmoc-Npg-OH.

| Amino acid used | Coupling agent/base and amount used |
|---|---|
| Fmoc-3COOHF(tBu)-OH | HATU (61.5 mmol), DIEA (65 mmol) |
| (65 mmol) | |
| Fmoc-Asp(tBu)-OH | HBTU (75 mmol) and DIEA (80 mmol) |
| (80 mmol) | |
| Fmoc-B5-OH (65 mmol) | HATU (61.5 mmol) and DIEA (65 mmol) |
| Fmoc-Npg-OH (75 mmol) | HATU (70 mmol) and DIEA (75 mmol) (×2) |
| (×2) | |
| Fmoc-Asp(tBu)-OH | HBTU (70 mmol) and DIEA (75 mmol) |
| (75 mmol) | |

After coupling Asp2, the B5/PyrS2 staple was closed by treating the resin with Hoveyda-Grubbs M720 catalyst (15.7 g, 25 mmol) and 1,4-benzoquinone (13.5 g, 125 mmol) in dichloroethane. The reaction proceeded at room temperature for 2 h with nitrogen bubbling, the catalyst was drained, and the treatment with M720 catalyst and 1,4-benzoquinone was repeated one more time before continuing with linear peptide synthesis.

| Amino acid/reagent used | Coupling agent/base and amount used |
|---|---|
| Fmoc-PL3-OH (75 mmol) | HBTU (75 mmol), DIC (75 mmol) |
| Ac2O (200 mmol) | DIEA (100 mmol) |

After N-terminal acetate capping, the PL3/B5 staple was closed by treating the resin with Grubbs catalyst M102 (20.6 g, 25 mmol) in dichloroethane at room temperature for 2 h with nitrogen bubbling. The catalyst solution was drained, and the treatment with Grubbs catalyst M102 was repeated another two times. The peptide was cleaved by treating the resin with 95:5 TFA:water (800 mL, v/v) for 2 hours, and the peptide was precipitated by pouring the cleavage cocktail into cold methyl tert-butyl ether. The precipitated peptide was filtered, washed with cold MTBE twice, and dried under vacuum. The peptide was first purified by dissolving in DMF, and loading onto a Luna C8 10 um 100 A column (flow rate: 20 mL/min) with a gradient of 45% to 75% acetonitrile in water (with 0.075% TFA) over 50 minutes. Product-containing fractions were dried, and the isolated peptide was subjected to a second purification, and was dissolved in 30% acetonitrile in water and loaded on a Kromasil C8 5 μm 100 A column (20 mL/min), first flowing 0.4M ammonium acetate over the column for 25 min, then eluting with a gradient of 50% to 70% acetonitrile in water with 0.5% acetic acid over 50 minutes. The product-containing fractions were lyophilized to provide I-66 (40:1 I-66:I-67, 4997 mg, 2.41 mmol, 4.8% yield) plus a second lot of I-66 (8:1 I-66:I-67, 2015 mg, 0.97 mmol, 1.9% yield). Ratio of I-66 and I-67 were assessed using HPLC: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and ratio is calculated based on peak area. As an example, in one run, retention time of I-66 is 15.3 min and retention time of I-67 is 16.2 min. In some embodiments, such a protocol provides improved resolution compared to a reference protocol by which I-66 and I-67 may elute as one peak or may otherwise not sufficiently separated. For example, by the general method for Table E2 I-66 and I-67 can be eluted together as the second peak and the mixture may be designated as I-67). Alternatively or additionally, ratios can also be assessed using other technologies, e.g., NMR. In some embodiments, such a preparation of I-66 or preparations corresponding thereto were assessed in various biological assays and was confirmed to possess various properties and activities; see, e.g., Examples 11-18. ¹H NMR of such a preparation of I-66 is presented in FIG. 6. As those skilled in the art reading the present disclosure will appreciate, FIG. 6 may contain peaks of certain impurities and/or residue ¹H in a NMR solvent. In some embodiments, NOE was observed between the peaks at about 5.45-5.6 and at about 5.2-5.35. Fractions can be further purified to provide improved purity.

In some embodiments, I-66 and/or I-67 prepared herein may be utilized as standard/reference to assess and/or characterize other compounds and/or other preparations of I-66 and/or I-67 (e.g., different batches prepared by the same or different methods). In some embodiments, I-470 is similarly prepared. In some embodiments, T-470 differs from T-66 in that T-470 has Glu2 and Glu5 while T-66 has Asp2 and Asp5.

In some embodiments, the present disclosure provides a compound having the structure of

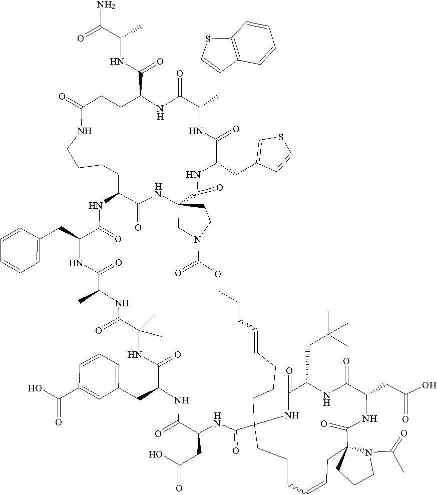 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, the present disclosure provides a compound having the structure of

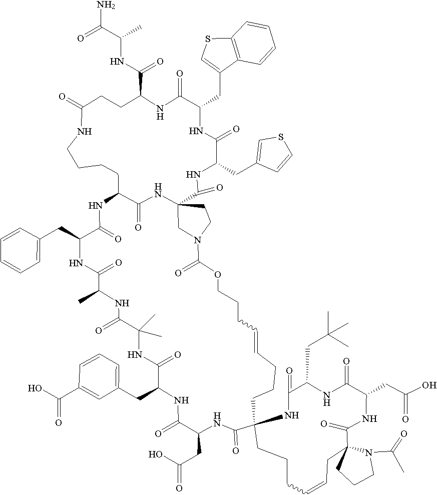 [SMILES: CC(=O)N1CCC[C@]12CC=CCCC[C@]1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, the present disclosure provides a compound having the structure of

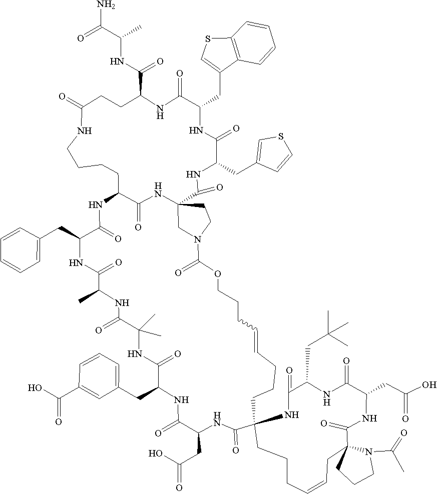 [SMILES: CC(=O)N1CCC[C@]12C/C=C\CCC[C@]1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

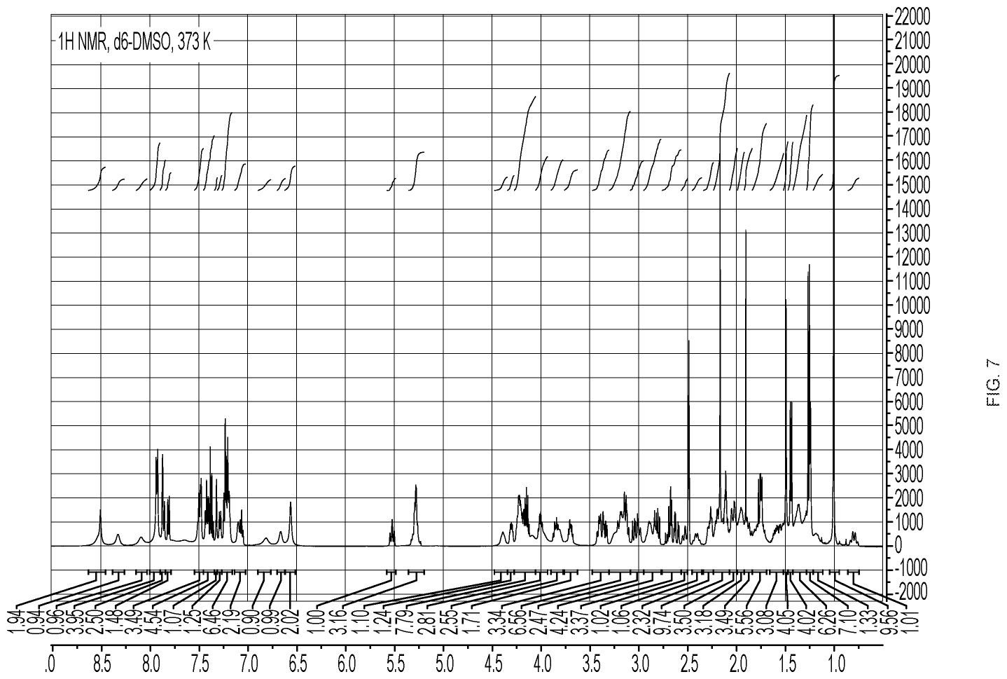

or a salt thereof. In some embodiments, the compound has the same retention time as I-66 prepared above under the same or comparable HPLC conditions. For example, in some embodiments, a HPLC condition is Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and a retention time of I-66 is about 15.3 min. In some embodiments, a HPLC condition separates I-66 and I-67. In some embodiments, when co-injected with a I-66 preparation described herein, the compound elute as a single peak as I-66. In some embodiments, the compound is characterized in that in its ¹H NMR spectrum, it shows peaks that overlap with those between about 5.1-5.7 in FIG. 6. In some embodiments, the compound is characterized in that in its ¹H NMR spectrum, it has the same peak pattern as FIG. 6 between about 5.1-5.7. In some embodiments, the compound has the same NMR spectra as I-66 under the same or comparable conditions. In some embodiments, the compound has the same ¹H NMR spectra as I-66 under the same or comparable conditions, e.g., DMSO-d6, 373 K. Those skilled in the art appreciate that peaks of certain ¹H, such as those bonded to nitrogen and oxygen, may shift in ¹H NMR for the same compound during different assessments. In some embodiments, the compound has such a structure that for its ¹H NMR, peaks of ¹H bonded to carbon are found in FIG. 6 under the same or comparable conditions (DMSO-d6, 373 K). In some embodiments, the compound has such a structure that its ¹H NMR peaks are found in FIG. 6 under the same or comparable conditions (DMSO-d6, 373 K). In some embodiments, the compound has such a structure that its ¹H NMR peaks for ¹H bonded to carbon atoms are found in FIG. 6 under the same or comparable conditions (DMSO-d6, 373 K). In some embodiments, integration of peak(s) in FIG. 6 that correspond(s) to each ¹H bonded to carbon in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. In some embodiments, integration of peak(s) in FIG. 6 that correspond(s) to each ¹H in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. An integration of FIG. 6 is presented in FIG. 7 as an example. Those skilled in the art appreciate that ¹H NMR results, e.g., chemical shifts, integration of peaks, etc., may have typical error ranges. In some embodiments, peaks corresponding to two or more ¹H may overlap. In some embodiments, such peaks may be integrated together for assessment of numbers of ¹H. In some embodiments, NMR of a preparation of I-66 described above are the same or comparable with or without the addition of the compound at a detectable level (e.g., the same amount of I-66) under the same or comparable conditions. In some embodiments, ¹H NMR of a preparation of I-66 described above are the same or comparable with or without the addition of the compound at a detectable level (e.g., the same amount of I-66, or about 0.1-10, 0.2-5, 0.5-2, 0.1, 0.2, 0.3, 0.4, 0.5, 0.6, 0.7, 0.8, 0.9, 1, 1.1, 1.2, 1.3. 1.4, 1.5, 2, 3, 4, 5, 6, 7, 8, 9, or 10, etc. of I-66). In some embodiments, ¹H NMR are considered the same or comparable when peaks corresponding to ¹H bonded to carbon have comparable chemical shift, peak shapes and/or integration. In some embodiments, peaks from impurities and solvents are properly excluded when comparing NMR. In some embodiments, peaks from impurities, solvents, ¹H bonded to oxygen, nitrogen, etc. are properly excluded when comparing NMR. In some embodiments, the compound has the same retention time as I-67 prepared above under the same or comparable HPLC conditions. For example, in some embodiments, a HPLC condition is Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and a retention time of I-67 is about 16.2 min. In some embodiments, a HPLC condition separates I-66 and I-67. In some embodiments, when co-injected with a I-67 preparation described herein, the compound elute as a single peak as I-67. In some embodiments, the compound has the same ¹H NMR peaks between about 5.0-6.0 as I-67 under the same or comparable conditions. In some embodiments, the compound has the same NMR spectra as I-67 under the same or comparable conditions. In some embodiments, the compound has the same ¹H NMR spectra as I-67 under the same or comparable conditions, e.g., DMSO-d6, 373 K. In some embodiments, NMR of a preparation of I-67 described above are the same or comparable with or without the addition of the compound at a detectable level (e.g., the same amount of I-67) under the same or comparable conditions. In some embodiments, ¹H NMR of a preparation of I-67 described above are the same or comparable with or without the addition of the compound at a detectable level (e.g., the same amount of I-67). In some embodiments, in a composition comprising the compound, ratio of the compound to a stereoisomer of the compound is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, ratio of the compound to each stereoisomer of the compound is independently about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, ratio of all compounds that are the compound or a salt thereof to all compounds that is a stereoisomer of the compound or a salt of the stereoisomer is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, for each stereoisomer of the compound, ratio of all compounds that are the compound or a salt thereof to all compounds that is a stereoisomer of the compound or a salt of the stereoisomer is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 2:1. In some embodiments, the ratio is about or at least about 3:1. In some embodiments, the ratio is about or at least about 4:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1.

In some embodiments, a preparation of I-66 comprises

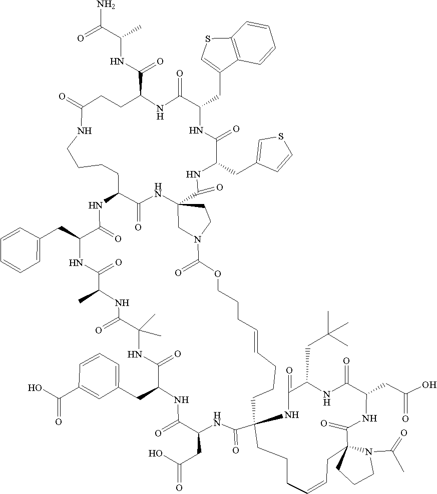 [SMILES: CC(=O)N1CCC[C@]12C/C=C\CCC[C@]1(CCC/C=C/CCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a preparation of I-67 comprises

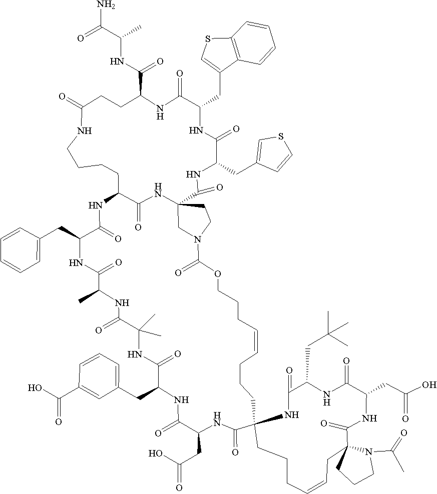 [SMILES: CC(=O)N1CCC[C@]12C/C=C\CCC[C@]1(CCC/C=C\CCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, a preparation of I-66 or a preparation of I-67 comprises a first compound

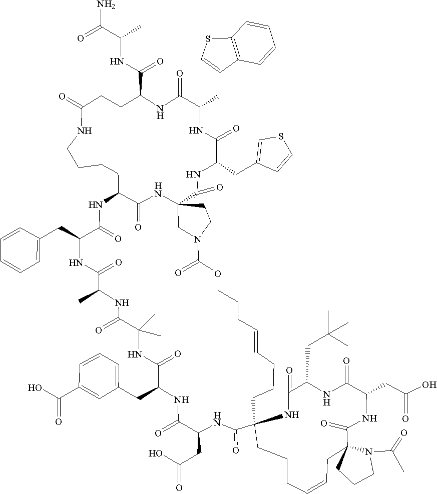 [SMILES: CC(=O)N1CCC[C@]12C/C=C\CCC[C@]1(CCC/C=C/CCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof, and a second compound

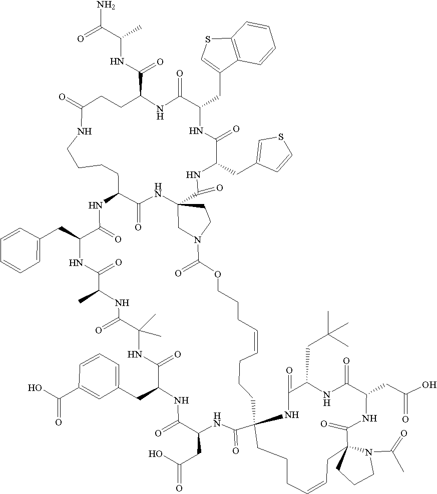 [SMILES: CC(=O)N1CCC[C@]12C/C=C\CCC[C@]1(CCC/C=C\CCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]

or a salt thereof. In some embodiments, in a preparation of I-66, ratio of the first compound to the second compound is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-66, ratio of all compounds that are the first compound or a salt thereof to all compounds that are the second compound or a salt thereof is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-67, ratio of the second compound to the first compound is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-67, ratio of all compounds that are the second compound or a salt thereof to all compounds that are the first compound or a salt thereof is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 2:1. In some embodiments, the ratio is about or at least about 3:1. In some embodiments, the ratio is about or at least about 4:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1. As utilized in the present disclosure, depending on the context, in some embodiments, a ratio is a molar ratio; in some embodiments, a ratio is a weight ratio; in some embodiments, a ratio is a volume ratio; and in some embodiments, a ratio is according to an assessment. For example, in some embodiments, when ratio of compounds are assessed using HPLC/UV, a ratio is of peak area of UV trace at a certain wavelength, e.g., 220 nm.

### Example 10. Provided Technologies can Provide High Selectivity

As confirmed below, in some embodiments, the present disclosure provides technologies with high selectivity for forming staples comprising olefin double bonds.

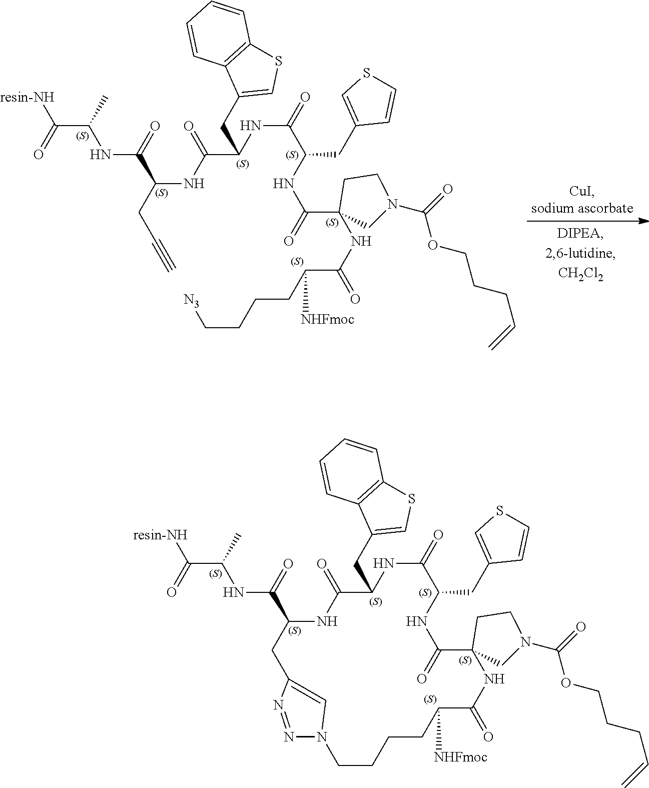

Fmoc-azidolysine-PyrS2-3Thi-BztA-propargylglycine-Ala-protide resin was synthesized using standard solid phase peptide synthesis procedures. The triazole staple was closed by treating the resin with one equivalent of copper (I) iodide, one equivalent of sodium ascorbate, ten equivalents of diisopropylethylamine, and ten equivalents of 2,6-lutidine in dichloromethane at room temperature for 48 h. The resin was washed for 5 min with DCM 2×, MeOH 1×, H₂O 2×, 50% H₂O/MeOH 2×, and MeOH 2×. In some embodiments, it was observed there was a small layer of insoluble material floating on top of the reactor, which was eliminated by aspiration through a hose connected to a pump. Then, continued with washes with NMP 2×, DCM 1×, and MeOH 1×.

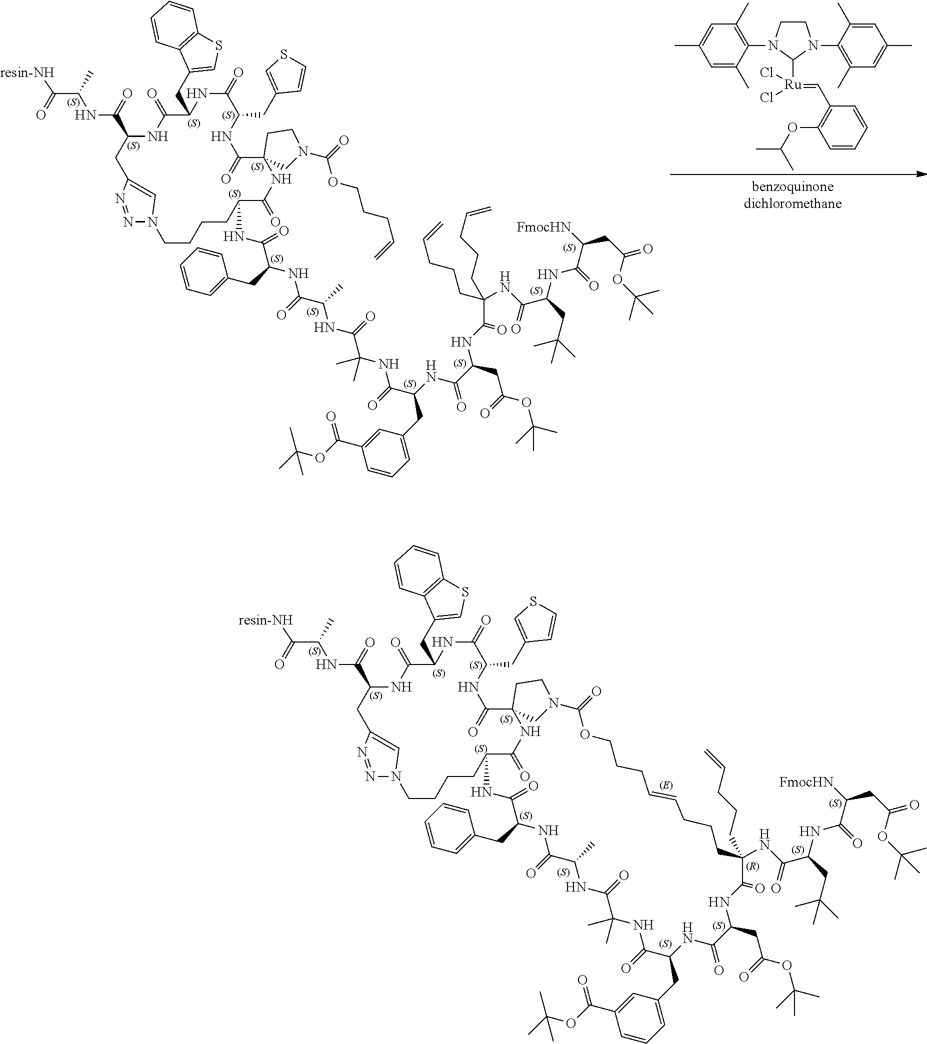

The cyclized product was elongated to Fmoc-Asp(OtBu)-Npg-B5-Asp(OtBu)-3COOHF(OtBu)-Aib-Ala-Phe-TriAzLvs*3-PyrS2-3Thi-Bzta-sAla*3-Ala-protide resin using standard solid phase peptide synthesis procedures. Afterwards, the resin was thoroughly washed with DCM 2×, NMP 1×, DCM 2×, MeOH 2×, DCM 1×, MeOH 1×, each for five minutes, then dried under a flow of nitrogen for 24 h to yield a gold color resin. The first staple was closed by treating the resin with 5 mol % Hoveyda-Grubbs M720 catalyst (CAS 301224-40-8) and 10 mol % benzoquinone in dichloromethane at reflux for 48 h. After 48 h, the catalyst solution was drained, the resin washed with dichloromethane 3×, dried, and then treated again with 5 mol % Hoveyda-Grubbs M720 catalyst (CAS 301224-40-8) and 10 mol % benzoquinone in dichloromethane at reflux for 48 h.

After analysis by LCMS, complete reaction was observed with no identifiable starting material. The desired product is detected in >95%, no other isomer by-product is observed. Double bond configuration is assigned based on analysis of NMR data, reported selectivity, etc., and can also be assessed by other technologies, e.g., crystallography.

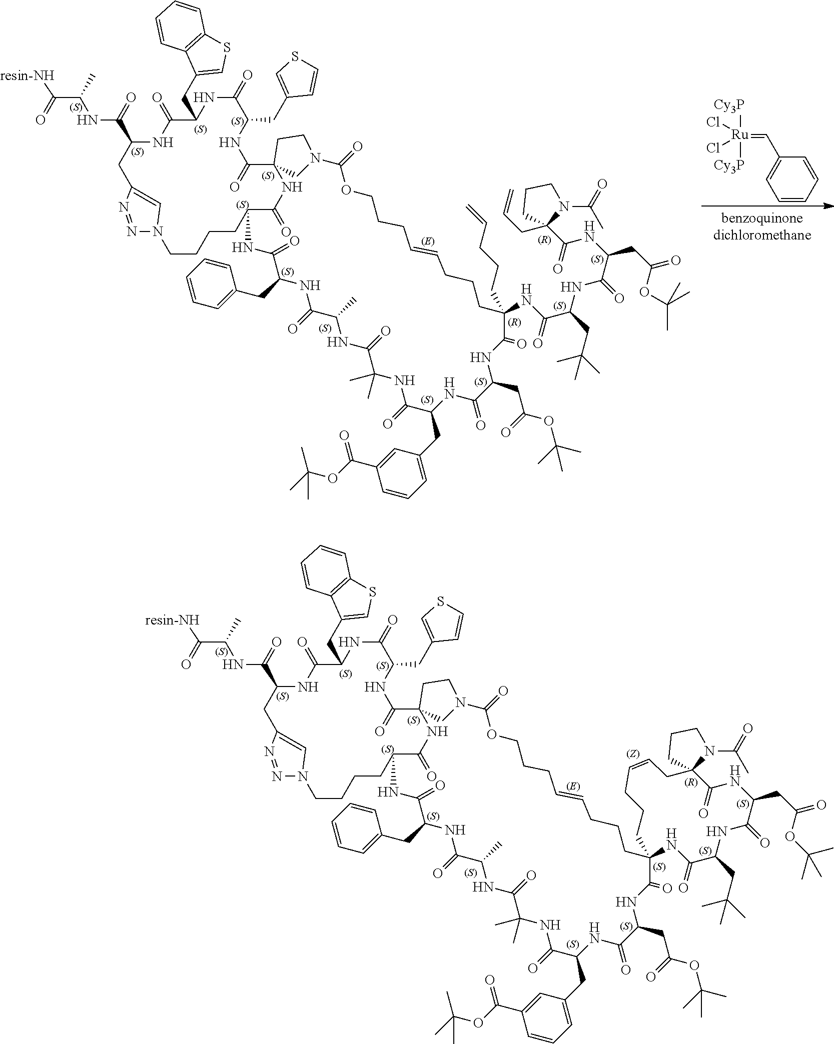

The above product was elongated to Ac-PL3-Asp(OtBu)-Npg-B5-Asp(OtBu)-3COOHF(OtBu)-Aib-Ala-Phe-TriAzLys*3-PyrS2-3Thi-Bzta-sAla*3-Ala-protide resin using standard solid phase peptide synthesis procedures. After acetyl capping of the N-terminus, the resin was thoroughly washed with DCM 2×, NMP 1×, DCM 2×, MeOH 2×, DCM 1×, MeOH 1×, each for five minutes, then dried under a flow of nitrogen for 24 h. The second staple was closed by treating the resin with 30 mol % Grubbs I M102 (CAS 172222-30-9) and 60 mol % benzoquinone in dichloromethane at reflux for 24 h. After 24 h, the catalyst solution was drained, the resin washed with dichloromethane 3×, dried, and then treated again with 30 mol % Grubbs I M102 (CAS 172222-30-9) and 60 mol % benzoquinone in dichloromethane at reflux for 24 h. The crude product was cleaved and deprotected, and was analyzed by LCMS and showed 82% (UV at, e.g., 210-400 nm) of I-335. Two more peaks of olefin isomers were detected on as 13% and 5% of total area by HPLC, respectively. Double bond configuration is assigned based on analysis of NMR data, reported selectivity, etc., and can also be assessed by other technologies, e.g., crystallography.

### Example 11. Provided Technologies can Provide Various Advantages

Among other things, provided technologies can provide various advantages. In some embodiments, provided technologies can provide improved target binding profiles and/or activity profiles. As confirmed below, stapled peptides, particularly I-66, can provide strong binding to beta-catenin and modulation of gene expression. Useful protocols for various assessments are described in the Examples.

| | | | Competition | | | | TCF | | qPCR | |
|---|---|---|---|---|---|---|---|---|---|---|
| IC50 | SPR Kd* | | Fluorescence Polarization | | NanoBRET | | reporter | | (AXIN2)** | |
| Peptide A | 20 | nM | 10 | nM | 3.0 | μM | 1.5 | μM | 1.4 | μM |
| I-66 | 700 | pM | <5 | nM | 1.1 | μM | 0.7 | μM | 0.3 | μM |
| I-470 | >5000 | nM | 7500 | nM | >20 | μM | >20 | μM | >20 | μM |

Peptide A: A stapled peptide Ac-PL3-Asp-Npg-B5-Asp-3COOHF-Ala-Ala-Phe-Leu-PyrS2-2F3MeF-BztA-Gln-NH2. PL3 and B5, and B5 and PyrS2 are stapled.
*T_{1/2} = 12.6 min for I-66.
**Reduction of AXIN2 transcripts after adminstration of agents to COLO320DM cells.

In some embodiments, it was confirmed, e.g., through biochemical competition assays, that provided technologies (e.g., I-66) can inhibit TCF/LEF transcription factor binding to β-catenin. In some embodiments, it was observed that provided technologies (e.g., I-66) compete with TCF1, TCF3, TCF4, LEF1, pAPC, mouse ECAD, human ECAD, etc. for beta-catenin interactions. In some embodiments, it was confirmed that provided technologies (e.g., I-66) can significantly reduce phospho-APC binding. In some embodiments, it was confirmed that provided technologies (e.g., I-66) can significantly reduce E-cadherin binding. In some embodiments, it was observed that there was little to no competitive effect for certain provided technologies, e.g., I-66, for ICAT, Axin or Bcl9. In some embodiments, interactions are dependent on phosphorylation, e.g., it has been reported that E-cadherin binding to beta-catenin is highly dependent on phosphorylation of up to eight Ser residues on E-cadherin.

In some embodiments, capabilities of provided technologies, e.g., binding to beta-catenin and/or disrupt its interactions (or lack thereof) with various partners were assessed and confirmed in cells, e.g., using a NanoBRET based assay in HEK293 cells. In some embodiments, it was observed that provided technologies, e.g., I-66, can potently inhibit such interactions without affecting cell viability.

Among other things, direct inhibition of endogenous beta-catenin/TCF interaction was confirmed by co-immunoprecipitation (co-IP) assays as described herein.

### Example 12. Provided Technologies can Modulate Transcription

Among other things, the present disclosure confirms that provided technologies can inhibit transcription of endogenous Wnt pathway target genes driven by the B3-catenin/TCF interaction. Among other things, it was confirmed that in DLD1 cells, peptide A and I-66 dose-dependently inhibited the expression of AXIN2 and SP5, two bona fide downstream genes of beta-catenin/TCF (peptide A: AXIN2 IC₅₀=9.3 uM, SP5 IC₅₀=9 uM; I-66: AXIN2 IC₅₀=1.6 uM, SP5 IC₅₀=1.3 uM). In some embodiments, no effect was observed on the expression of CTNNB1 for peptide A and I-66 in DLD1 cells under a tested condition. Reduction of expression level of a canonical beta-catenin target AXIN2, was also observed in COLO320DM cells (peptide A: IC₅₀=1.4 uM; I-66: IC₅₀=0.3 uM) while I-470 had no or very little or non-significant effect.

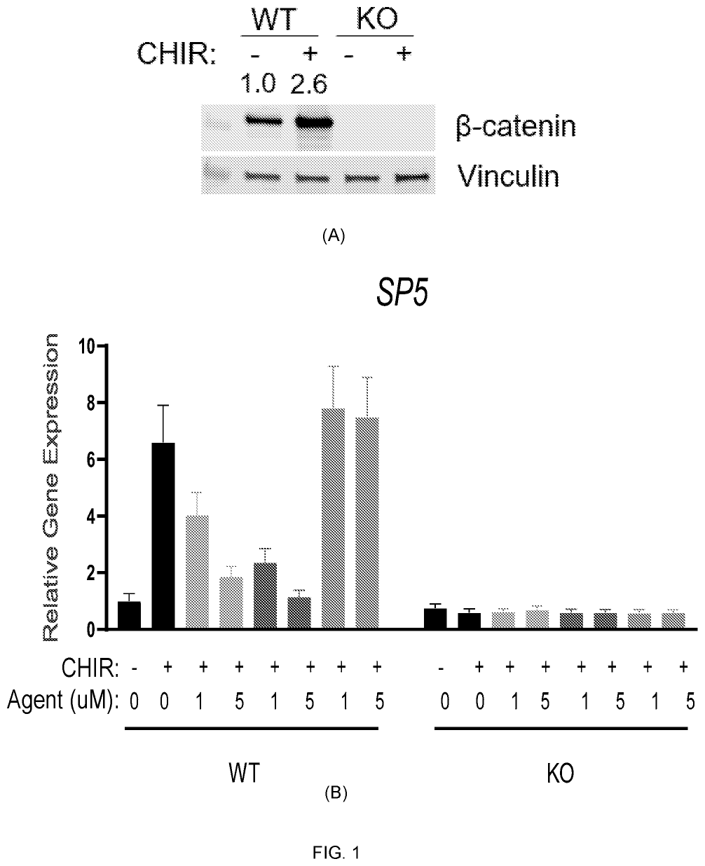

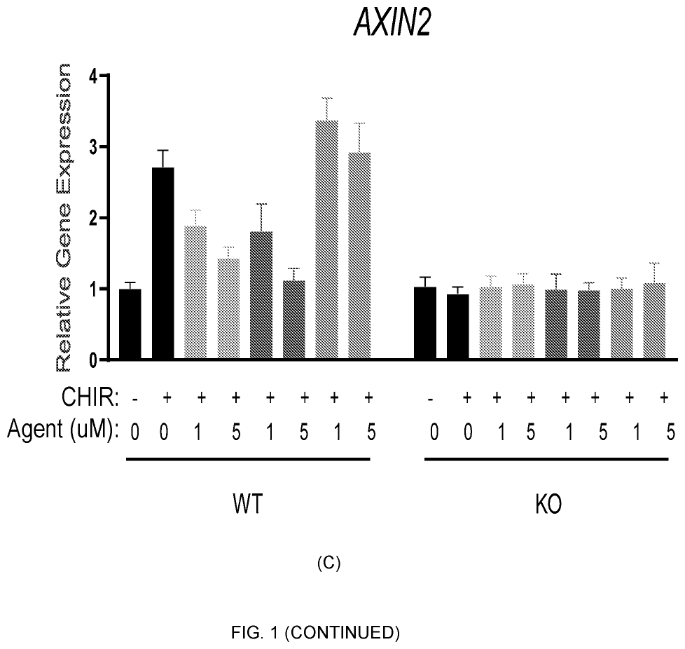

Among other things, provided technologies can modulate transcription and levels of various transcripts, in some embodiments, with certain types and/or levels of selectivity. For example, in various systems, e.g., HAP1 isogenic lines (+/−CTNNB1 knockout), provided technologies can modulate level of expression and/or activity of a nucleic acid, e.g., a gene, a transcript, a polypeptide, and/or a product thereof selectively in systems comprising or expressing beta-catenin. For example, in some embodiments, provided technologies inhibit beta-catenin driven transcription selectively in HAP1 WT cells. Certain data are presented in FIG. 1 as examples. In cells expressing WT beta-catenin (WT), 24-hour CHIR treatment increased beta-catenin protein levels more than two-fold, and peptide A and I-66 treatment significantly reduced the expression of AXIN2 and SP5 as measured by qPCR (by about 3- and 8-fold, respectively, with I-66). Inhibition of RNF43 expression by peptide A and I-66 was also observed. In beta-catenin KO cells, neither CHIR nor peptide A and I-66 affected the expression of AXIN2, SP5, or RNF43. No reduction of transcription in WT cells was observed for I-470. In some embodiments, treatment with provided peptides, e.g., I-66 at 10 um for 72 or 144 hours, was observed to not significantly affect beta-catenin stability in cells with functioning beta-catenin destruction complex by western blot.

### Example 13. Provided Technologies can Reduce Beta-Catenin Levels in Nuclei

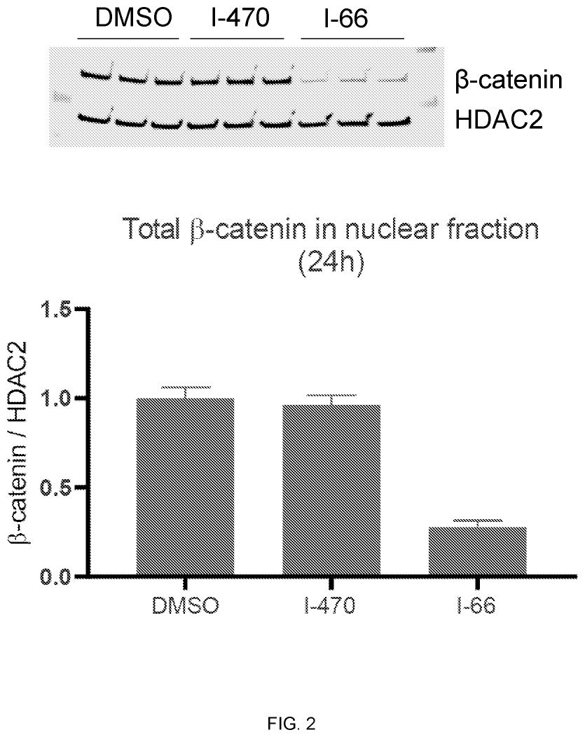

In some embodiments, provided technologies can reduce level of beta-catenin in nuclei. In some embodiments, provided technologies can block beta-catenin nuclear localization. In some embodiments, provided technologies can reduce level of beta-catenin nuclear translocation. For example, as confirmed in FIG. 2, provided technologies can reduce levels of nuclear beta-catenin in various cells including COLO320DM cells (10 uM, 24 hr). Reduction of nuclear localization was also confirmed by immunofluorescence imaging. In some embodiments, it was observed that after 24-hr I-66 treatment, nuclear beta-catenin levels were reduced by over 70% compared to untreated cells. Similar results were obtained after 24- and 48-hr treatments.

### Example 14. Provided Technologies can Inhibit Proliferation and Induce Cell Cycle Arrest

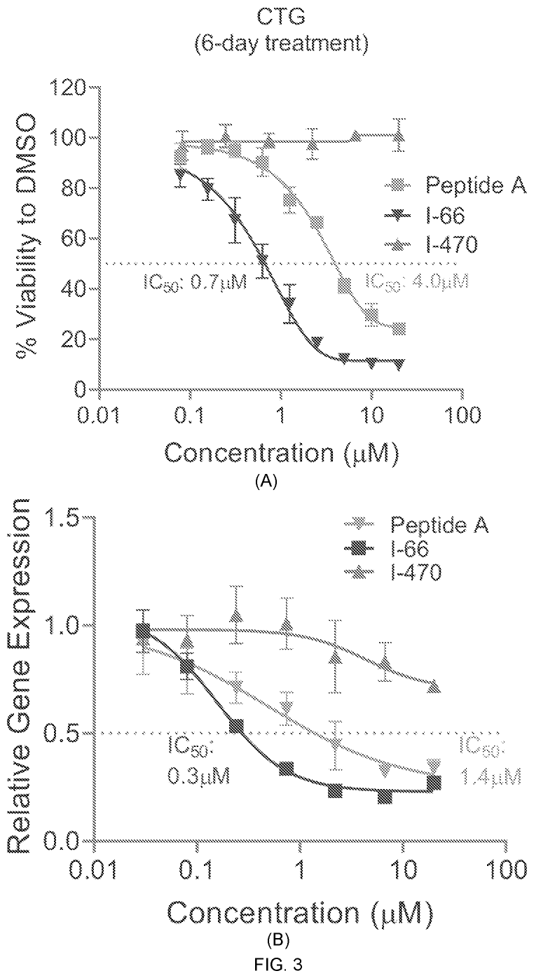

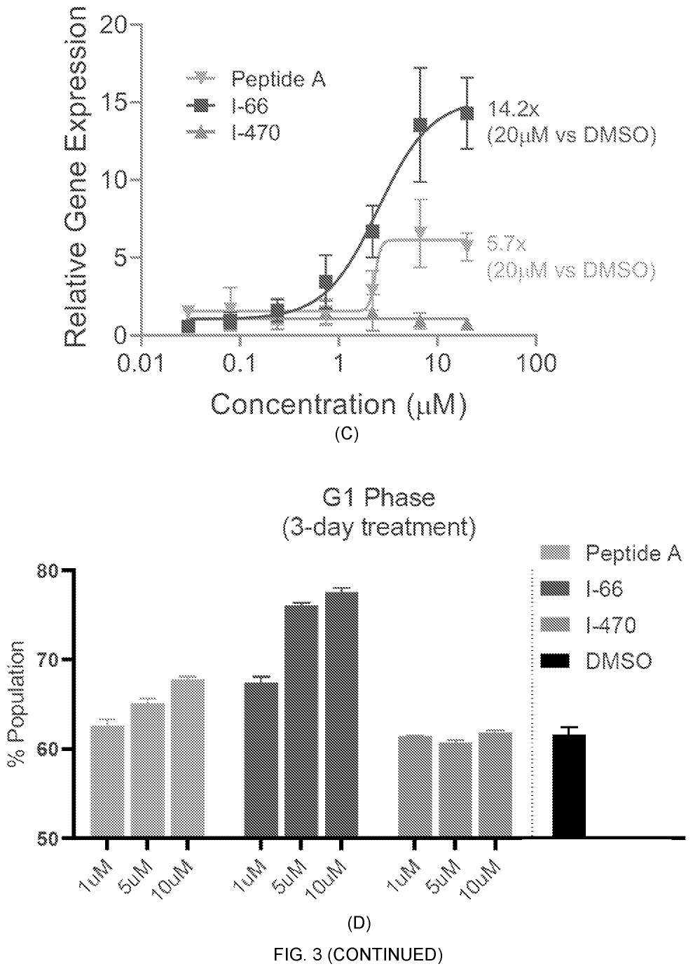

As described herein, among other things, provided technologies can inhibit proliferation of various cells including various cancer cells. In some embodiments, provided technologies modulate WNT specific transcription. In some embodiments, provided technologies induce cell cycle arrest. In some embodiments, provided technologies induce G1 cell cycle arrest. In some embodiments, provided technologies increased proportion of cells in G1 phase of cell cycle. As confirmed in FIG. 3, provided technologies can inhibit proliferation of COLO320DM, which is a colorectal cell line comprising various mutations such as APC, TP53, etc., modulate WNT specific transcription such as of AXIN2 and CXCL12, and induce G1 cell cycle arrest. In some embodiments, it was confirmed that provided technologies can reduce proportion of cells in S phase of cell cycle. In some embodiments, it was confirmed that provided technologies can significantly down-regulate Cyclin D2 and up-regulate p27. In some embodiments, changes of various genes, e.g., AXIN2, CXCL12, etc., were observed to be consistent with changes with shRNA-knockdown. Two separate doxycycline (dox)-inducible shRNAs were utilized to knockdown (KD) CTNNB1 in COLO320DM cells. Decreased expression of AXIN2 and increased expression of CXCL12 were observed. CTNNB1-KD also significantly reduced the proliferation of COLO320DM cells.

In some embodiments, an assessment was performed as follows. On day 0, cells were seeded in cell culture media (RPMI1640, 4% FBS) in a 96-well plate at desired density, typically at 1000 cells/well. On day 1, 10 mM agent stock solution (in DMSO) was first serially diluted into DMSO at 1:2 ratio, followed by diluting with cell culture media at two times of the final concentrations. Finally, agent-containing media were introduced to cell culture wells already having the same volume of cell culture media. Cells were incubated with agents for desired days before lysed for CellTiter-Glo® Luminescent Cell Viability Assay according to the manufacture instruction (Promega, G7570). Luminescent signal was obtained from a microplate reader (GloMax, Promega). Cell viability data was expressed as % relative to DMSO control wells.

### Example 15. Provided Technologies can Provide Robust Anti-Tumor Effects In Vivo

As described herein, provided technologies are useful for treating various conditions, disorders or diseases including cancer. Among other things, the present Example confirms that provided technologies can provide in vivo efficacy as demonstrated in various animal models. Certain useful models and/or protocols are described below as examples. Those skilled in the art reading the present disclosure appreciate that various models for various cancers may be utilized to assess provided technologies and confirm their effects in accordance with the present disclosure.

COLO320DM human colorectal cancer cells (ATCC, CCL-220), which comprise various mutations, e.g., APC and TP53, etc., were expanded in RPMI 1640 media (10% FBS) and inoculated subcutaneously, 10⁷ cells per animal in 100 uL PBS/Matrigel (1:1) mixture, to male NU/J mice (JAX#2019) at 8 weeks of age. When the average tumor size reached 150 mm³, mice were randomized into 3 cohorts (n=10) and treated with vehicle (1% Tween 80/99% 10 mM PBS pH 7.4), I-66 (30 mg/kg), and I-66 (75 mg/kg) via intraperitoneal injection, once every 4 days for 5 doses.

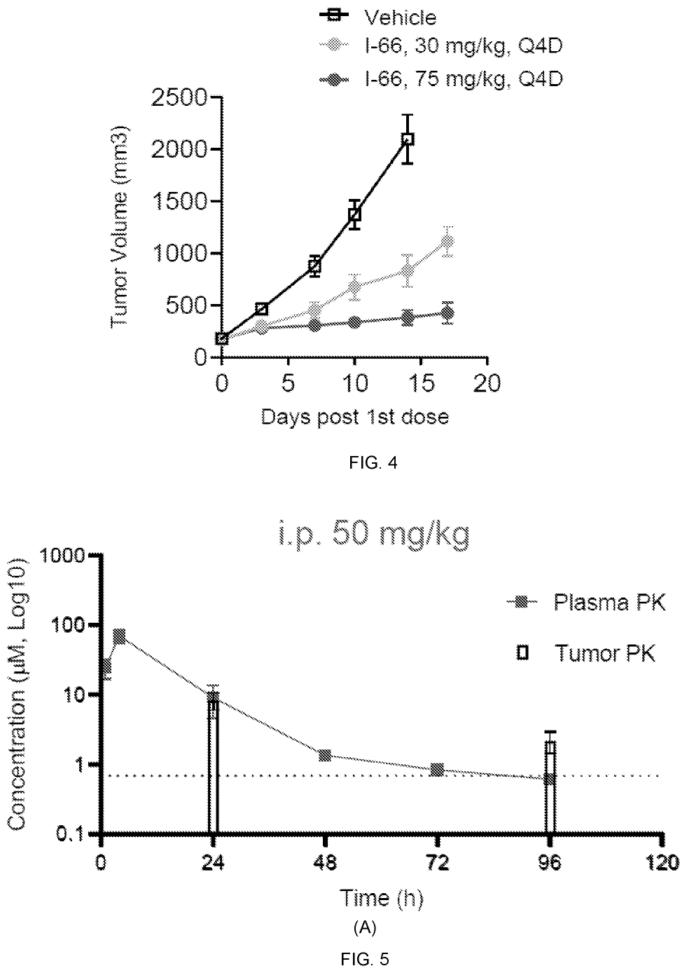

Tumor volume was measured by electronic caliper every 2-3 days until tumor volume reached 2000 mm³ and estimated as (length×width²)/2. Body weights were weighed every 2-3 days and represented as % body weight=(BWi−BW0)/BW×100% (BWi: body weight at day i, BW0: body weight at day 0). Tumor growth inhibition was calculated as, TGI %=[1−(TVi−TV0)/(TVvi−TVv0)]×100% (TVi: average tumor volume of a dosing group on day i, TV0: average tumor volume of a dosing group on day 0, TVvi: average tumor volume of a vehicle group on day i, TVv0: average tumor volume of a vehicle group on day 0). Animals were euthanized by CO₂ asphyxiation on the designated terminal day for each study, and plasma, tumors, tissues, etc., were excised for further analysis. Certain data are presented in FIG. 4 as examples.

As confirmed, technologies of the present disclosure can provide robust anti-tumor efficacy. For example, in some embodiments, in COLO320DM xenograft model, I-66 was dosed once every four days, and the treatment led to significant tumor growth inhibitions (TGI) of 66% and 89% at 30 and 75 mg/kg on day 14, respectively. At 75 mg/kg, an initial loss in body weight was observed after the first dose but recovered over time.

In some embodiments, transcriptional effects of pathway inhibition in vivo were assessed. For example, in some embodiments, several PD markers from COLO320DM tumors obtained at the end of the efficacy study (e.g., Day 18) were assessed. In agreement with in vitro and single-dose in vivo data, both AXIN2 and CXCL12 were dose-dependently regulated by provided technologies, e.g., I-66, in tumors (for AXIN2, down-regulation and for CXCL12, up-regulation), confirming durable target gene modulation. Reduction of mouse NOTUM level in plasma was also observed. In some embodiments, NOTUM may be utilized as a biomarker, e.g., for assessing a treatment, selecting patient population, determining whether to continue treatment, etc. In some embodiments, assessment of human plasma samples from normal and patients, e.g., colorectal cancer patients, confirms that NOTUM levels are correlated with stage of diseases and may be suitable for clinical applications, e.g., as a target engagement biomarker.

### Example 16. Provided Technologies can be Delivered In Vivo

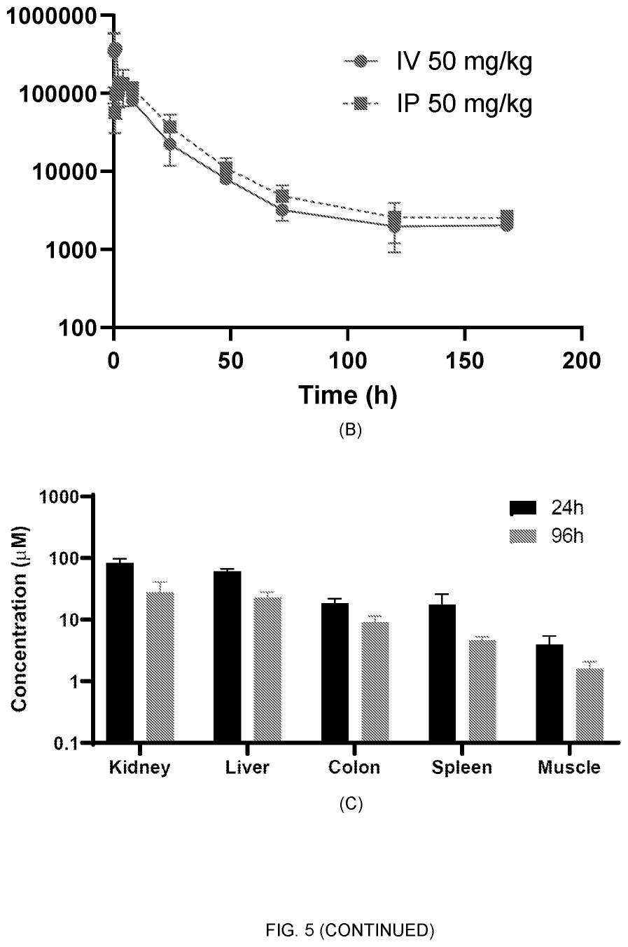

Among other things, various suitable in vivo pharmacokinetic and/or pharmacodynamic properties and/or activities have been confirmed. For example, as confirmed in FIG. 5, (A), provided technologies can be effectively delivered to tumors. As shown, prolonged tumor exposure to I-66 was observed after a single dose, and tumor exposure was about 2˜10 fold above in vitro IC50 for proliferation. It was also observed that I-66 tumor PK exceeded plasma PK at 96 hr time point. Further, I-66 provided significantly longer time periods during which tumor exposure was about or above in vitro IC50 for proliferation when compared to, e.g., Peptide A. A useful protocol for assessment is described below as an example.

Experiments were carried out under an Institutional Animal Care and Use Committee-approved protocol, and institutional guidelines for the proper and humane use of animals were followed.

For COLO320DM, male NU/J mice (6-8 weeks of age) were utilized, and mice were randomized when average tumor volume reached 300 mm³. For IP dosing, agents were formulated in 10 mg/mL arginine and 6% PEG400 phosphate (pH 7.4) formulation.

Concentrations of agents in biological samples were measured by LC-MS/MS (Triple Quad 6500+). Using analytical grade chemicals and solvents, 25 ng/ml Tolbutamide in acetonitrile (ACN, LS120-4, Fisher Scientific) was used as internal standards. 8 uL of plasma or tissue lysate was used for LC method with mobile phase A (1% formic acid (FA, LS118-4, Fisher Scientific) in H₂O) and mobile phase B (0.1% FA in ACN), 0.6 ml/min flow rate in Waters ACQUITY UPLC BEH C18 2.1*50 mm, 1.7 μm column. The calibration curve was generated using 5-5000 ng/mL agent, e.g., I-66, in mouse plasma and tissue homogenates. MS was conducted by electrospray ionization and multi reaction monitor scans. PK parameters such as plasma maximum concentration (Cmax), and AUC were analyzed by noncompartmental model 200 of Phoenix WinNonlin 8.3, using the linear/log trapezoidal method.

Additional data confirm well-behaved pharmacokinetic (PK) profiles of provided technologies. See, for example, FIG. 5, (B) and data below.

### Certain PK Parameters of I-66 in Mouse by 2-Compartmental Analysis.

| PK Parameters | IV | IP |
|---|---|---|
| Cmax(ng/mL) | 498654 | 152000 |
| T_{1/2} (h) | 28.71 | 41.7 |
| Tₘₐₓ (h) | NA | 6.67 |
| Vdₛₛ (L/kg) | 0.452 | NA |
| Cl (mL/min/kg) | 0.30 | NA |
| Tₗₐₛₜ (h) | 168.00 | 168.00 |
| AUC₀₋ₗₐₛₜ (ng · h/mL) | 2741991 | 2971069 |
| AUC_{0-inf} (ng · h/mL) | 2826191 | 3124730 |
| Bioavailability (%) | NA | 105.0 |

In some embodiments, broad tissue distribution was observed. For example, as shown in FIG. 5, (C), I-66 was detected in all samples shown. In some embodiments, durable tissue residence was confirmed at least between 24- and 96-hr post-injection. In some embodiments, a single intraperitoneal (IP) dose of I-66 and I-470 at 100 mg/kg demonstrated comparable plasma AUC in mouse.

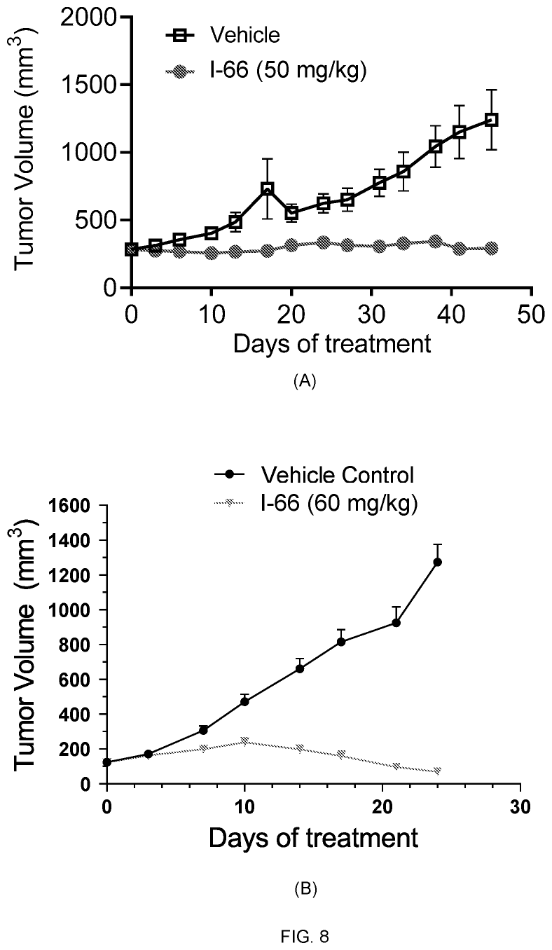

Robust and durable anti-tumor effects by provided technologies were confirmed in additional tumor models. In some embodiments, such effects were observed in a Patient-Derived Xenograft (PDX) cancer models. In some embodiments, a model is a mouse PDX colon cancer model. In some embodiments, this model has APC mutations (Tyr935Ter His1490LeufsTer20) and high AXIN2 expression. In some embodiments, for AXIN2 expression, LogCPM is about 2.5 or greater. Among other things, strong anti-tumor activities and durable tumor growth inhibition were confirmed. For example, TGI=103% on day 45 was observed for animals dosed at 50 mg/kg. No significant body weight loss was observed. Certain data are presented in FIG. 8, (A), as examples. In some embodiments, provided technologies were assessed in a mouse model carrying a patient-derived xenograft colorectal tumor. Again, robust anti-tumor effects were confirmed. Certain data are presented in FIG. 8, (B), as examples. Vehicle vs. I-66, p=0.008. Mutation profile of model: APC mutant, KRAS WT. IP dosing Q4D, n=10/group. In some embodiments, animals were dosed and/or observed for longer time, e.g., beyond 24 days. No significant body weight loss was observed. For both PDX assessments, vehicle is 10 mM sodium phosphate dibasic, 6% w/w PEG-400, 10 mg/mL L-Arginine.

Among other things, data in various Examples confirmed that provided technologies can provide robust PK properties, strong anti-tumor efficacy and on-target transcriptional modulation in vivo.

### Example 17. Provided Technologies Modulate Expressions In Vivo

As described herein, provided technologies can modulate expression of various nucleic acids and/or levels of products thereof, e.g., RNA transcripts, polypeptides, etc. For example, tumor RNA-sequencing analysis confirmed that I-66 can provide, among other things, strong on-target Wnt/beta-catenin pathway modulation in COLO320DM tumors. Certain negatively enriched gene sets are presented below as examples. In some embodiments, a negatively enriched gene is CCND2, WNT5B, AXIN2, NKD1, WNT6, DKK1, OR DKK4. It is noted that both negatively and positively enriched gene sets were observed. Among other things, the present disclosure provides technologies for assessing efficacy of a method, e.g., a treatment, comprising assessing expression of one or more negatively and/or positively enriched genes. In some embodiments, if expression profiles of one or more genes are negatively and/or positively enriched as identified herein, a method may be considered to have efficacy, and/or administration (e.g., of provided technologies such as stapled peptides, compositions, etc.) to a subject can continue.

Top Negatively Enriched Gene Sets include BCAT_GDS748-UP, BCAT.100-UP.V1-UP, HALLMARK_WNT_BETA_CATENIN_SIGNALING, RASHI_RESPONSE_TO_IONIZING_RADIATION_1, REACTOME_RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, HALLMARK_TNFA_SIGNALING_VIA_NFKB. I-66 vs. I-470. i.p. 30 mg/kg, 48 hr post single dose. NES −1.7 or smaller. FDR q-value 0.02 or smaller.

Comparable concentrations of I-66 and I-470 were found in tumors (e.g., in an assessment, 4266 and 5181 ng/gram, respectively) at 48-hr post-dose. As confirmed, GSEA revealed multiple Wnt/beta-catenin and MYC related gene sets ranked as the top hits among the negatively enriched gene sets. Consistent with cell-based data, this result confirms that provided technologies e.g., I-66, can provide strong on-target Wnt/beta-catenin pathway modulation in tumors as shown here in COLO320DM tumors.

In some embodiments, the present disclosure provides technologies for identifying regulated nucleic acids and/or products thereof including gene sets, and how they are regulated. In some embodiments, patterns of regulation of one or more nucleic acids and/or products thereof, or groups of nucleic acids and/or products thereof such as gene sets, are useful for selecting patient populations for treatment or continued or adjusted treatment (e.g., dose levels, regimens, etc.).

A useful protocol is described below as an example.

RNAseq Preparation. For RNA-seq of cell line grafted tumors, library preparation and sequencing were performed with a suitable kit, e.g., TruSeq stranded mRNA library kit on Novaseq S4 Platform, in some embodiments, with PolyA enrichment.

RNAseq Data Analysis. In some embodiments, sequence reads were trimmed to remove possible adapter sequences and nucleotides with poor quality using Trimmomatic v.0.39. The trimmed reads were mapped to the Homo sapiens GRCh38 reference genome available on ENSEMBL using the STAR aligner v.2.7.7a. For grafted tumor samples, host reads were removed with XenofilteR. Unique gene hit counts were calculated by using featureCounts from the R Subread package v.2.4.2. Read filtering, normalization, and differentially expression analysis was performed with the edgeR package v.4.0.2 in R. Genes with an adjusted p-value <0.01 and absolute log 2 fold change >1 were called as differentially expressed genes for each comparison. Genes that are differentially expressed in at least one comparison were used in heatmap and clustering analysis. Gene expression was normalized to fold changes over a reference, e.g., DMSO, controls at the same time. The R pheatmap package v.1.0.12 was used to make heatmap and for hierarchical clustering of genes, with correlation as similarity measure. For enrichment analysis, GSEA v4.1.0 was run with gene list ranked by fold change with the MSigDB database v7.3. In some embodiments, Venn diagram was produced with ggvenn v.0.1.9, where the p value of overlap was calculated with hypergeometric test in R v4.1.2. Those skilled in the art appreciate that other software, programs and/or algorithms may be utilized.

Time- and dose-dependent effects of provided technologies, e.g., I-66, on expression were also observed in COLO320DM cells through RNA seq. It was confirmed that treatment by provided technologies, e.g., I-66, led to both time- and dose-dependent effects on COLO320DM transcriptional profile. In some embodiments, at 1 uM, 0, 107 and 359 differentially expressed genes (DEGs) were detected at 6-, 24- and 48-hr post treatment, respectively. At 10 uM, 73, 876 and 1271 DEGs, respectively, were found at the three time points. RNAseq data from shRNA-expressing cells after 3-day dox treatment were also assessed. In some embodiments, it was observed that CTNNB1-KD by shRNA and provided technologies, e.g., I-66, led to a consistent transcriptome change in COLO320DM (R2=0.68, p<2.2E-16).

To assess impacts of provided technologies at pathway levels, Gene Set Enrichment Analysis (GSEA) was utilized to identify significantly enriched Hallmark gene sets (FDR<0.05) in cells treated by provided technologies, e.g., I-66. Dox-induced CTNNB1-KD and shRNA-resistant CTNNB1 cDNA (shR-cDNA) rescue cell lines were included as comparators. GSEA identified a Hallmark Wnt/beta-catenin gene set that includes AXIN2, DKK4, NDK1 and other canonical Wnt target genes was significantly down-regulated at 10 uM at 6 hr (FDR=0.001), and at all 3 doses (1, 3, and 10 uM) at 24 hr and 48 hr (e.g., WNT_BETA_CATENIN_SIGNALING). MYC targeted gene sets and cell cycle related gene sets (E2F and G2M) were also significantly down regulated in treated cells by provided technologies, e.g., I-66, first observed at 24 hr and also found at 48 hr (e.g., MYC_TARGETS_V1, MYC_TARGETS_V2, E2F_TARGETS, G2M_CHECKPOINT, etc.). These gene set changes were confirmed by dox-induced CTNNB1-KD and were reversed by expressing shR-cDNA, indicating they were indeed downstream effects of beta-catenin. For those gene sets enriched by CTNNB1-KD (i.e. coagulation, myogenesis, interferon), treatments by provided technologies, e.g., I-66, largely showed consistent trends at 24 hr and 48 hr. In some embodiments, in certain assessments certain dose/time point combinations may not reach statically significance. In some embodiments, the present disclosure provides technologies for modulating expression levels and/or functions of one or more nucleic acids, e.g., genes, in one or more such gene sets and/or pathways, and/or products encoded thereby. In some embodiments, the present disclosure provides technologies for modulating expression and/or functions of such gene sets and/or pathways. In some embodiments, levels are reduced. In some embodiments, levels of expression and/or functions may be utilized as bio-markers as described herein, e.g., for assessing a treatment, for monitoring treatment progress, for selection of patients for a treatment or continuation of a treatment, etc. In some embodiments, it was observed that glycolysis and cholesterol gene sets were negatively enriched by genetic perturbation but not by treatment of I-66. Among other things, on-target inhibition of beta-catenin signaling through disruption of its interaction with TCF/LEF transcription factors by provided technologies was confirmed in various embodiments.

### Example 18. Additional Characterization and Assessment of Provided Technologies

As described herein, various technologies may be utilized to characterize and assess provided technologies in accordance with the present disclosure. Certain technologies and results are described herein as examples. Those skilled in the art appreciate that these example technologies may be adjusted or modified.

Crystallography. In some embodiments, structures, interactions, etc. are characterized and assessed using X-Ray crystallography and structure determination. The following protocol is provided as example. In some embodiments, beta-catenin (Human Armadillo Repeat Domain 1-12 (aa146-aa665))/I-66 complex was concentrated to 9.9 mg/mL and sitting drop trays were setup at 4° C. In some embodiments, a complex was crystallized with 0.49M (NH₄)₂SO₄, 0.38M Li₂SO₄, 0.10 M Na₃Cit, pH=6.00 at 4° C. Crystals were cryo protected followed by flash-freezing in liquid nitrogen. Diffraction datasets were collected at 100 K at beamlines PXII and X10SA of the SLS. Molecular replacement solutions were obtained using PHASER. In some embodiments, complete models were built through iterative cycles of manual model building in COOT and structure refinement using both REFMAC and PHENIX. In some embodiments, atomic coordinates and structure factors are deposited in the Protein Data Bank. Among other things, the structure confirmed that various amino acid residues in I-66 interact with various amino acid residues in beta-catenin, for example: PL3-1 with Val349, Asp2 with Lys312 and Gly307, Npg3 with Tyr306, Asp5 with Asn387 and Trp383, 3COOHF-6 with Lys345, Ala8 with Trp383, Phe9 with Lys345 and Trp383, 3Thi-12 with Trp-383 and Asn-415, and BztA-13 with Gln-379, Leu-382, Val-416, Asn-415, and Trp-383.

Competitive Fluorescence Polarization. In some embodiments, interactions are assessed using competitive fluorescence polarization. The following protocol is described as an example. In some embodiments, compounds at 10 mM in DMSO were serially diluted 1:3 in DMSO for a total of 11 concentrations using the Mosquito LV (SPT Labtech, Covina, CA), then diluted 1000-fold in buffer (50 mM HEPES, pH 7.5, 125 mM NaCl, 2% glycerol, 0.5 mM EDTA, 0.05% v/v pluronic acid) in duplicate by the Mosquito LV into a black polystyrene 384-well plate (Corning, Corning, NY). Probe solution was prepared by mixing 10 nM full-length beta-catenin (Uniprot ID P35222) with 10 nM fluorescently labeled (5FAM) peptide representing TCF4 residues 10-53 (Uniprot ID Q9NQB0) peptide. The plate was incubated protected from light for 1 hour at room temperature prior to read. Reads were performed on a CLARIOstar plate reader (BMG Labtech, Cary, NC) with excitation at 485 nm, emission at 525 nm, and cutoff at 504 nm. Data were fitted to a 1:1 binding model with hill slope using an in-house script.

SPR. In some embodiments, SPR may be utilized for characterizing or assessing interactions, bindings, etc. The following protocol is described as an example. In some embodiments, SPR experiments were performed on a Biacore™ 8K (Cytiva, Marlborough, MA) instrument at 25° C. Compounds were diluted into running buffer (50 mM Tris pH 8.0, 300 mM NaCl, 2% glycerol, 0.5 mM TCEP, 0.5 mM EDTA, 0.005% Tween-20, 1% DMSO). Compounds were diluted to 1 uM or 10 uM (e.g., peptide A, I-66, etc.) and serially diluted 1:3 for 9 concentrations and two blanks. Biotinylated beta-catenin residues 134-665 (Uniprot ID P35222) was immobilized to the active surface of the sensor chip for 25 seconds at 10 mL/min using the Biotin CAPture Kit, Series S (Cytiva) and compounds were injected over the reference and active surfaces for 180 seconds at 65 mL/min then allowed to dissociate for 400 seconds. Results were analyzed using the Biacore™ Insight Evaluation software, with double referencing and fitted to a 1:1 binding affinity model.

ABA Competition Assays. In some embodiments, an ABA competition assay is utilized to characterize or assess a provided technology. The following protocol is described as an example. In some embodiments, SPR experiments were performed on a Biacore™ S200 (Cytiva) instrument at 25° C. beta-catenin binding regions of APC, E-cadherin, and AXIN1, ICAT were expressed and purified from E coli. In some embodiments, BCL9 utilized was a synthesized peptide comprising the amino acid sequence interacting with beta-catenin. In some embodiments, APC was treated with kinase to generate phosphorylated-APC (pAPC) as reported. In some embodiments, peptide sequences were obtained from Protein Data Bank (PDB) or Uniprot: TCF1 (Uniprot#P36402, aa 15-60), TCF3 (PDB: 1G3J), TCF4 (PDB:1JDH), LEF1(Uniprot#Q9UJU2 aa 14-62), pAPC (PDB: 1TH1), Mouse E-cadherin (PDB: 117X), Human E-cadherin (Uniprot#12830, aa 732-882), ICAT (PDB: 1LUJ), AXIN1 (PDB: 1QZ7), BCL9 (PDB: 2GL7). beta-catenin binding partners (proteins or peptides) were diluted into running buffer (50 mM Tris pH 8.0, 300 mM NaCl, 2% glycerol, 0.5 mM TCEP, 0.5 mM EDTA, 0.005% Tween-20, 0.09% DMSO). Biotinylated beta-catenin residues 134-665 (Uniprot#ID P35222) was immobilized to the active surface of the sensor chips for 25 seconds at 10 mL/min using the Biotin CAPture Kit, Series S (Cytiva) for an immobilization level -200RU. Compounds (e.g., I-66, I-470 (as control), etc.) were diluted to 500 nM in running buffer and injected over the surface for 30 seconds at 90 mL/min seconds. In some embodiments, appropriate concentrations for each beta-catenin binding partners were chosen to ensure >90% fractional occupancy for a compound (e.g., I-66) and they were injected 67 s at 90 uL/min over the surface plus or minus a compound (e.g., I-66) using SPR ABA injection protocol. In some embodiments, results were double-referenced and analyzed using the Biacore™ Insight Evaluation software to assess competition.

Cell lines and cell culture. As those skilled in the art appreciate, cell lines may be obtained from various sources including commercial vendors. For example, HAP1 isogenic pair (HZGHC001062c011) can be obtained from Horizon Discovery (Waterbeach, United Kingdom), and many cell lines can be obtained from the American Type Culture Collection (ATCC). Various technologies may be utilized to culture cells in accordance with the present disclosure. Cells were routinely cultured in their preferred media according to vendor recommendations. In some embodiments, cells harboring inducible shRNA constructs were maintained in appropriate media with tetracycline-free fetal bovine serum (631101, Clontech Laboratories). Various reagents can be obtained from commercial sources. For example, CHIR99021 can be purchased from R&D System (#4423). In various embodiments, experiments were typically performed at 4% FBS condition. In some embodiments, experiments performed at other FBS concentrations were indicated.

NanoBRET. In some embodiments, NanoBRET is utilized to characterize or assess provided technologies. The following protocol is described as an example. In some embodiments, a bioluminescence resonance energy transfer (BRET)-based assay was established in HEK293 cells, using NanoBRET constructs to assess beta-catenin/TCF4 interaction (Promega, Madison, WI) according to the manufacturer protocol. TCF4 was fused to a luminescent donor NanoLuc™ and beta-catenin was fused to a HaloTag® NanoBRET™ 618 Ligand (HL) as an acceptor. Briefly, on day 1, cells were transfected with NanoBRET plasmids according to the manufacturer protocol and 30 mM (LiCl (, L7026, Sigma) was added to cell culture media to stabilize beta-catenin. On day 2, fresh media containing compounds and LiCl was added to the cells. On day 3, Nanoluciferase substrate (N157B, Promega) was added to the cells, and the fluorescence emission from HL measured using a GloMAX instrument (Promega) with emission at 460 nm (donor) and 618 nM (acceptor). Cell viability of these cells was monitored alongside the NanoBRET analyses using the luminescence-based assay, CellTiter-Glo (CTG) (G7570, Promega).

TCF Reporter and Negative Reporter Assays: In some embodiments, TCF report assays are utilized to characterize or assess provided technologies. TCF reporter assays including kits have been reported and can be utilized in accordance with the present disclosure. In some embodiments, in a TCF reporter assay, reporter cell line was generated by using TCF/LEF luciferase reporter lentivirus (79787, BPS Bioscience), and a negative control reporter line was generated using a control luciferase lentivirus (79578, BPS Bioscience). Parental DLD1 cells were transfected with the lentivirus and followed by 3-day puromycin selection. Single clone was selected for both reporter assays. Compounds were incubated with reporter cells for a suitable period of time, e.g., 24 hr. After that, luciferase activity was measured using the Bright-Glo Luciferase Assay System (E2620, Promega). Cell viability was monitored using the luminescence-based cell viability assay, CTG (G7570, Promega). Both peptide A and I-66 inhibited luciferase activity in a dose-dependent manner (IC₅₀ 1.5 μM and 0.7 μM, respectively) without affecting cell viability. Neither showed any activity in a negative control reporter assay, where luciferase was under the control of a minimal TATA promoter.

Western Blotting. Various technologies may be utilized to detect or quantify polypeptides. In some embodiments, wester blotting is utilized. The following protocol is described as an example. Cells were harvested in 1×RIPA buffer (BP-115, Boston Bioproducts) containing phosphatase and protease inhibitor cocktail (5872S, Cell Signaling Technologies). Tumors were homogenized in 4% SDS buffer using a polytron homogenizer(P000062-PEVO0-A, Bertin). Equal amount of proteins were resolved on precast 4-20% SDS-PAGE gels (5671093, Bio-Rad), and subsequently transferred onto nitrocellulose membrane for detection. In some embodiments, primary antibodies were probed overnight at 4° C., membranes were washed with TBST, and incubated with appropriate secondary antibodies for 1 hour. Subsequently membranes were washed with TBST and visualized using Odyssey imaging system (LI-COR). In some embodiments, primary antibodies used were beta-catenin (8480, Cell Signaling Technology), anti-vinculin mouse antibody (V9131, Sigma-Aldrich), anti-Cyclin D2 (3741, Cell Signaling Technology), anti-p27 (3686, Cell Signaling Technology), anti-HDAC2 (5113, Cell Signaling Technology). Depending on polypeptide to be assessed, other antibodies may be utilized. In some embodiments, secondary antibodies used were Alexa Fluor 680 secondary antibody (A32734, Thermo Fisher Scientific) and anti-mouse Alexa Fluor 800 secondary antibody (A32730, Thermo Fisher Scientific). In some embodiments, protein bands were visualized and quantified using the Odyssey CLx Imaging System (Li-Cor) and ImageStudio software (Li-Cor).

RT-qPCR. In some embodiments, RT-qPCR is utilized for assessing transcripts or RNA. The following protocol is described as an example. In some embodiments, tumors were homogenized in RLT buffer followed by total RNA was isolated using RNAeasy kit (74104, Qiagen) according to manufacturer's protocol. Cells were washed with ice cold PBS and total RNA was extracted using RNeasy Kit (74104, Qiagen). cDNA conversion was performed immediately following RNA extraction using High-Capacity cDNA Reverse Transcription Kit (4374966, ThermoFisher). cDNA was stored in the −20° C. until use. qPCR was performed using TaqMan Universal PCR Master Mix (ThermoFisher) and TaqMan Probes (ThermoFisher) on a QuantStudio 7 Flex Real-Time PCR System (ThermoFisher) with technical duplicates. Relative gene expression levels were monitored using the Taqman Gene Expression probes for AXIN2 (Hs00610344 m1, ThermoFisher), SP5 (Hs01370227-mH, ThermoFisher), RNF43 (Hs00214886-m1, ThermoFisher), NOTUM (Hs00991061-m1, ThermoFisher), CXCL12 (Hs03676656-mH, ThermoFisher). Reactions used Advanced Fast Master Mix (4444557, ThermoFisher) and CT values were normalized to ACTB (4325788, ThermoFisher) as the endogenous control. Other suitable probes may be utilized in accordance with the present disclosure.

Co-Immunoprecipitation. In some embodiments, co-immunoprecipitation is utilized to assess interactions, complexing, etc. The following protocol is described as an example. In some embodiments, in cells, e.g., DLD1 cells, peptide A and I-66, but not I-470, dose-dependently blocked beta-catenin/TCF4 interaction as detected by Western blotting. In some embodiments, provided peptides traverse cell membrane and/or inhibit beta-catenin/TCF interaction. In some embodiments, provided peptides directly bind to intracellular beta-catenin. In some embodiments, it was observed that various peptides, e.g., I-66, did not affect beta-catenin/E-cadherin interaction, e.g., in DLD1 cells. In some embodiments, for co-IP experiments, DLD1 cells were treated with compounds for a period of time, e.g., 4 hours. Cell pellets were washed twice with PBS and re-suspended in IP-MS Cell Lysis Buffer provided with the Pierce MS-Compatible Magnetic IP Kit (90409, ThermoFisher (containing Halt protease/phosphatase inhibitor (78440, ThermoFisher)) and sonicated for 2×10 seconds (30% amplitude) followed by incubation on ice for 10 min to achieve cell lysis. Lysates were then centrifuged for 10 min at 14000×g to pellet debris. Protein concentration was determined using a Pierce BCA Assay Kit (23225, ThermoFisher), and final protein concentration was adjusted to about 1 mg/mL using lysis buffer. For each condition, 1 mL of lysate was added to a 96-deepwell plate and incubated with rabbit monoclonal beta-catenin antibody (8480, Cell Signaling Technology) 1:50 dilution or rabbit isotype control for 16 hr at 4° C. in a thermomixer at 300 rpm. Protein-antibody complexes were captured using magnetic protein A/G beads according to the Pierce MS-Compatible Magnetic IP Kit (90409, ThermoFisher) protocol using a Kingfisher Flex Magnetic Particle Processor (ThermoFisher). Briefly, 30 μL of protein A/G magnetic beads were added to each lysate and incubated for 1 hr at room temperature. The beads were then washed 3× in buffer B (5188-5217Agilent), and 2× in Buffer B (5185-5988, Agilent), followed by elution for 10 min in 100 μL of elution buffer. In some embodiments, eluates were dried in a vacuum concentrator (SPD120, ThermoFisher) and re-suspended in 50 μL of Preomics LYSE buffer and digested according to the protocol of PreOmics iST 96X kit (P.O.00027, PreOimics).

shRNA. In some embodiments, shRNA is utilized for gene knock-down. In some embodiments, shRNAs constructs were made in the pLKO-Tet-On lentiviral vector backbone. In some embodiments, specific sequences targeted were: shNT: 5′-CAACAAGATGAAGAGCACCAA-3′; sh637: 5′-CTATCAAGATGATGCAGAACT-3′; and sh1487: 5′-TCTAACCTCACTTGCAATAAT-3′. The cDNA construct directing overexpression of CTNNB1 was made in pLVX-EF1a-IRES-neo lentiviral vector, which was derived from a pLVXEF1a-IRES-puro vector (Clontech, 631988) by exchanging the selection cassettes. The cDNA construct was untagged. All constructs were confirmed by sequencing.

Lentiviral technologies. In some embodiments, lentivirus-based constructs (e.g., reporter, shRNAs, cDNA overexpression, etc.) were made using a standard protocol from, e.g., The RNAi Consortium (TRC) from the Broad Institute (http://portals.broadinstitute.org/gpp/public/resources/protocols). In some embodiments, shRNA viruses were titered on individual target cell lines and infected at MOI no greater than 0.7. In some embodiments, cDNA overexpression viruses were infected at higher MOI, where possible. To infect, cells were centrifuged for 1 hr at 2,250 rpm in the presence of the viruses and 8 ug/mL polybrene (H9268, Sigma). Media was preplaced after the spin, and drug selection was added 24 hr later (e.g., puromycin or neomycin, as appropriate). Selection was typically carried out until uninfected control cells were all dead.

2D Colony Formation. In some embodiments, 2D colony formation is utilized for assessing cell growth or proliferation. In some embodiments, COLO320DM cells were plated into 6-well tissue culture plate at 6000 cells/well. Next day, cells received fresh media with or without 200 ng/mL doxycycline (dox, S5159, Selleck). Media, with or without dox, was changed every 3 days until cells without dox reached 50-70% confluency. Cells were fixed with Glyoxal (411, ANATECH) for 24h at 4° C. and then stained with 0.5% crystal violet (031-04852, WAKO) for 1 hour at RT. Extra stain was removed with multiple water washes before imaging by Odyssey CLx Imaging System (Li-Cor) and ImageStudio software (Li-Cor).

Proliferation Assay. In some embodiments, various proliferation assays are utilized to characterize or assess provided technologies. In some embodiments, on day 0, cells were seeded in cell culture media in a 96-well plate at desired density, typically at 1000 cells/well. On day 1, 10 mM compound stock solution was first serially diluted into DMSO, followed by diluting with cell culture media at two times of the final concentrations. Finally, compound-containing media were introduced to cell culture wells already having the same volume of cell culture media. Cells were incubated with compounds for desired days before lysed for CTG according to the manufacture instruction (G7570, Promega). Luminescent signal was obtained from a microplate reader (GloMax, Promega).

Cell Cycle Analysis. Various technologies may be utilized to assess effects on cell cycles by provided technologies in accordance with the present disclosure. For example, in some embodiments, COLO320DM cells were prepared for cell cycle analysis using the Click-iT EdU kit (Thermo Fisher C10337) to monitor cell proliferation and FxCycle Violet (Thermo Fisher R37166) for quantitation of DNA per manufacturer's instructions. In some embodiments, flow analysis was performed on a BD LSRFortessa Flow Cytometry. In some embodiments, compensation was conducted between the FITC and BV421 channels. In some embodiments, DNA undergoing active synthesis incorporated EdU dye and was visible in the FITC channel. In some embodiments, DNA content incorporated the FxCycle Dye and was visible in the BV421 channel. Cells were gated into three distinct populations: low FITC and low BV421 signal (G1 population), high FITC (S population), and low FITC and high BV421 (G2 population). Data analysis was conducted using FlowJo software (BD Life Sciences).

RNAseq Preparation. In some embodiments, RNAseq is utilized to assess expression of various nuclei acids including genes. The following describes a process as an example. In some embodiments, for RNA-seq of COLO320DM cell line treated with compounds, library preparation and sequencing reactions were conducted at GENEWIZ, LLC. (South Plainfield, NJ). RNA-seq libraries were prepared using the Illumina TruSeqstranded Total RNA protocol with subsequent PolyA enrichment. On average 25 million 2×150 base pair reads were produced per sample with Illumina HiSeq. For RNA-seq of shRNA treated samples, library preparation and sequencing were performed by Mingma Technologies (Shanghai, China) with TruSeq stranded mRNA library kit on Novaseq S4 Platform with PolyA enrichment. On average over 60 million 2×150 base pair reads were produced per sample. For RNA-seq of cell line grafted tumors, library preparation and sequencing were performed by Fulgent Gentetics (Houston, TX) with TruSeq stranded mRNA library kit on Novaseq S4 Platform with PolyA enrichment.

RNAseq Data Analysis. Various technologies may be utilized to analyze RNAseq data in accordance with the present disclosure. In some embodiments, sequence reads were trimmed to remove possible adapter sequences and nucleotides with poor quality using Trimmomatic v.0.39. The trimmed reads were mapped to the Homo sapiens GRCh38 reference genome available on ENSEMBL using the STAR aligner v.2.7.7a. For grafted tumor samples, host reads were removed with XenofilteR. Unique gene hit counts were calculated by using featureCounts from the R Subread package v.2.4.2. Read filtering, normalization, and differentially expression analysis was performed with the edgeR package v.4.0.2 in R. In some embodiments, genes with an adjusted p-value <0.01 and absolute log 2 fold change >1 were called as differentially expressed genes for each comparison. Genes that are differentially expressed in at least one comparison were used in heatmap and clustering analysis. In some embodiments, gene expression was normalized to fold changes over DMSO controls at the same time. In some embodiments, the R pheatmap package v.1.0.12 was used to make heatmap and for hierarchical clustering of genes, with correlation as similarity measure.

In some embodiments, for enrichment analysis, GSEA v4.1.0 was run with gene list ranked by fold change with the MSigDB database v7.3. Venn diagram was produced with ggvenn v.0.1.9, where the p value of overlap was calculated with hypergeometric test in R v4.1.2.

Nuclear Protein Extraction. In some embodiments, nuclear protein is extracted for assessment. The following protocol is described as an example. Cytoplasmatic and nuclear protein extraction was performed using NE-PER™ Nuclear and Cytoplasmic Extraction Reagents (78833, Thermo Fisher Scientific) supplemented with Halt™ Protease and Phosphatase Inhibitor Cocktail (78442, Thermo Fisher Scientific) according to manufacturer protocol. Cytoplasmic and nuclear extracts were stored in the −80° C. until use.

Immunofluorescence Staining. In some embodiments, immunofluorescence staining is utilized to detect, quantify, characterize or assess a polypeptide. The following protocol is described as an example. In some embodiments, COLO320DM cells were seeded at initial density of 40,000 cells/chamber in Nunc™ Lab-Tek™ II Chamber Slide™ System (154534PK, Thermo Fisher Scientific) overnight in RPMI and 10% FBS. The following day, media was replaced with RPMI with 4% FBS containing 0.1% DMSO, 10 uM I-66 or I-470. After 24 hr compound treatment, cells were washed with PBS and fixed with 10% Neutral-Buffered Formalin (HT501128-4L, Sigma-Aldrich) for 15 minutes at room temperature. Cells were then simultaneously permeabilized and blocked with 0.1% Triton x-100 (X¹⁰⁰-100ML, Sigma-Aldrich) and 10% donkey serum (D9663-10 ML, Sigma-Aldrich) in PBS for 1 hr at room temperature. Afterwards, cells were then incubated at 4° C. overnight using an anti-p-catenin rabbit primary antibody (8480, Cell Signaling Technology) diluted 1:100 (v/v) in 0.1% Triton x-100/10% Donkey Serum/PBS permeabilization/blocking buffer. Cells were then simultaneously incubated with an anti-rabbit Alexa Fluor 488 secondary antibody (A32790, Thermo Fisher Scientific) diluted 1:1000 (v/v) and phalloidin Alexa Fluor 647 (A30107, Thermo Fisher Scientific) diluted 1:200 (v/v) in 0.1% Triton x-100/10% Donkey Serum/PBS permeabilization/blocking buffer for 1 hr at room temperature. Those skilled in the art appreciate that other primary and/or secondary antibodies can also be utilized. Afterwards, cells were then incubated with DAPI (D3571, Thermo Fisher Scientific) diluted 1:10000 in PBS for 15 minutes at room temperature. Cells were washed with PBS for 3×5 minutes after every step. Chamber walls were then removed and cells were mounted using ProLong™ Glass Antifade Mountant (P36980, Thermo Fisher Scientific) with a cover glass overnight at room temperature. Cells were imaged using a Zeiss LSM 710 confocal laser scanning system. Confocal images were analyzed using FIJI/ImageJ.

Animal studies. In some embodiments, animal models are utilized to ass provided technologies. Experiments were typically carried out under an Institutional Animal Care and Use Committee-approved protocol, and institutional guidelines for the proper and humane use of animals were followed. The following protocol is described as an example. For example, for COLO320DM xenograft assessment, male NU/J mice (6-8 weeks of age) were used, and mice were randomized when the average tumor volume reached 300 mm³. For IP dosing, compounds were formulated in 10 mg/mL arginine and 6% PEG400 phosphate (pH 7.4) formulation. In some embodiments, PDX murine model was established in athymic nude-Foxn1 nu female mice, for example, in some embodiments, with CRC patient tumor with APC mutation (Tyr935Ter), amplified HER2, wild type KRAS and beta-catenin and high AXIN2 expression. Tumor volume was measured by electronic caliper every 2-3 days until tumor volume reached 2000 mm³ and estimated as (length×width²)/2. Body weights were weighed every 2-3 days. Tumor growth inhibition (TGI) was calculated as, TGI %=[1−(TVi−TV0)/(TVvi−TVv0)]×100% (TVi: average tumor volume of a dosing group on day i, TV0: average tumor volume of a dosing group on day 0, TVvi: average tumor volume of a vehicle group on day i, TVv0: average tumor volume of a vehicle group on day 0). Animals were euthanized by CO₂ asphyxiation on the designated terminal day, and plasma, tumors, tissues, etc. were excised for further analysis.

Compound Quantification. In some embodiments, LC-MS is utilized for quantifying various compounds including stapled peptides. In some embodiments, concentrations of compounds, e.g., stapled peptides, in biological samples were measured by LC-MS/MS (Triple Quad 6500+). Using analytical grade chemicals and solvents, 25 ng/mL tolbutamide in acetonitrile (ACN, LS120-4, Fisher Scientific) was used as internal standards. 8 uL of plasma or tissue lysate was used for LC method with mobile phase A (1% formic acid (FA, LS118-4, Fisher Scientific) in H₂O) mobile phase B (0.1% FA in ACN), 0.6 ml/min flowrate in Waters ACQUITY UPLC BEH C18 2.1*50 mm, 1.7 um column. The calibration curve was generated using 5-5000 ng/mL stapled peptides (e.g., I-66, I-470, etc.) in mouse plasma and tissue homogenates. In some embodiments, MS was conducted by electrospray ionization and multi reaction monitor scans. In some embodiments, PK parameters such as plasma maximum concentration (Cmax), and AUC were analyzed by noncompartmental model 200 of Phoenix WinNonlin 8.3, using the linear/log trapezoidal method.

Plasma NOTUM by Mass Spectrometry. The following protocol is described as an example for Plasma NOTUM by Mass Spectrometry. In some embodiments, plasma samples were collected from mice grafted with COLO320DM tumors for shotgun proteomic analysis. In some embodiments, plasma samples were first depleted of the most abundant proteins, e.g., the top 3, using Multiple Affinity Removal Column, Mouse-3 (4.6×50 mm, 5188-4217, Agilent), an immunoaffinity, HPLC-based methodology. In some embodiments, removal of highly abundant proteins allows for detection of medium to low abundant proteins by shotgun proteomics. An UltiMate™ 3000 Rapid Separation Quaternary System (ThermoFisher) was configured as recommended in the operational guidelines. For each sample 45 uL was added to 180 uL of Agilent Buffer A (5185-5987) and centrifuged in 0.22 um spin filters (5185-5990, Agilent) for 1 minute at 16,000×g. 180 uL of each sample was injected onto the Mouse-3 column. Elution of low/high abundant proteins from the Mouse-3 column was monitored at 280 nm by a UV detector. Low abundant proteins were collected by a fraction collector. The final volume for each low abundant fraction was about 1 mL. Each fraction was concentrated using a 5 kDa MWCO spin column concentrators (5185-8991, Agilent) for 60 minutes at 3,400×g. Sample volumes were approximately 50-80 uL after this step was completed. Samples were digested with trypsin (25200114, ThermoFisher) using the PreOmics iST 96X digestion kit (P.O.00027) protocol.

For LC-MS/MS analysis of peptide mixtures, separations were carried out using an UltiMate 3000 RSLCnano System (ThermoFisher). Peptides were resolved based on hydrophobicity using an EASY-Spray PepMap RSLC C18, 2 um, 100 A, 500 mm×75 μm I.D. column thermostatically controlled at 50° C. and at 300 nL/min flow rate with a linear gradient from 2% to 30% acetonitrile containing 0.1% FA for a total duration of 90 minutes. After the gradient portion of the chromatogram the column was washed with 99% acetonitrile containing 0.1% FA for 14 minutes and equilibrated with 2% acetonitrile containing 0.1% FA for 26 minutes. In some embodiments, MS analyses were performed on Q Exactive HF-X (ThermoFisher) in the positive-ion mode using an EASY-Spray source (ES903, ThermoFisher). The instrument was operated with the spray voltage of 1.9 kV, an ion transfer capillary temperature of 250° C. and S lens RF level of 40%. One high resolution FTMS scan of 120,000 resolution including 1 micro scan with maximum injection time of 200 ms was followed by 18 dependent FTMS MS/MS scans of 15,000 resolution with maximum injection time of 28 ms. The dependent MS/MS scans were performed using an isolation width of 1.4 m/z for the parent ion of interest. The isolated multiple charged ions (2, 3, 4) were activated using the HCD normalized collision energy of 28 eV. To prevent an ion from triggering a subsequent data-dependent scan after it has already triggered a data-dependent scan dynamic exclusion of 30 s was enabled.

In some embodiments, protein identification and quantification was performed with Proteome Discoverer v 2.5.0.400 using the Sequest HT algorithm. For plasma proteomics experiments, database searches were performed using both Homo sapiens (sp_canonical TaxID=9606) (v2021-07-30) & Mus musculus databases (sp_canonical TaxID=10090) (v2021-09-30). Database searches were performed with the following settings: trypsin digestion, precursor mass tolerance of 20 ppm, fragment mass tolerance of 0.02 Da, static modification: carbamidomethyl, dynamic modification: oxidation/N-terminal Met-loss. Protein abundances were normalized to total protein amount in each sample, and normalized protein abundance for NOTUM was extracted. Comparison of mean normalized NOTUM abundances between groups was performed by one-way ANOVA followed by Tukey's HSD. For co-immunoprecipitation experiments, database searches were performed with a Homo sapiens database and the same settings as for plasma proteomics above. Mean normalized abundances of beta-catenin binding partners were compared between conditions by one-way ANOVA followed by Tukey's HSD. In some embodiments, mass spectrometry proteomics data are deposited to the ProteomeXchange Consortium (http://proteomecentral.proteomexchange.org), e.g., via a PRIDE partner repository.

### Example 19. Compounds Comprising Thioether Staples can Provide Various Activities

As described herein, various staples can be utilized in accordance with the present disclosure. In some embodiments, a staple comprises —S—. In some embodiments, a staple comprises two —S—. In some embodiments, two —S— are not bonded to each other. In some embodiments, a staple is a thioether staple. Various such staples are described herein, e.g., those having the structure of -L^{s1}-S-L^{s2}-S—, wherein each of L^{s1}, L^{s2} and L^{s3} are independently as described herein. In some embodiments, L^{s1} and L^{s3} are independently from an amino acid residue, e.g., cysteine, homocysteine, alpha-methylcysteine, penicillamine, etc. In some embodiments, each is —CH₂—. In some embodiments, two thiol groups are linked by reacting with a compound having the structure of LG-L^{s2}-LG or a salt thereof, wherein each of LG and L^{s2} is independently as described herein. Various such compounds are as described herein. In some embodiments, such a staple is a (i, i+4) staple. In some embodiments, such a staple is closer to a C-terminus. In some embodiments, such a staple is between X¹⁰ and X⁴. Among other things, the present disclosure confirms that stapled peptides comprising such staples can provide various activities, e.g., binding to target (e.g., beta-catenin), inhibition of tumor growth, etc.). Certain stapled peptides and data are presented below as examples. C-1 is Ac-PL3-Asp-Npg-B5-Asp-3COOHF-Ala-Ala-Phe-Leu-PyrS2-2F3MeF-BztA-Gln-NH2, wherein PL3 and B5, and B5 and PyrS2 are stapled. In some embodiments, C-1 is the second production peak/fraction on HPLC (see, e.g., Table E2) of a preparation of Ac-PL3-Asp-Npg-B5-Asp-3COOHF-Ala-Ala-Phe-Leu-PyrS2-2F3MeF-BztA-Gln-NH2.

| | beta-catenin TCF4 | beta-catenin TCF4 | | beta-catenin TCF4 | beta-catenin TCF4 |
|---|---|---|---|---|---|
| | competition FP | DLDI cell reporter | | competition FP | DLD1 cell reporter |
| ID | assay IC50 (nM) | assay IC50 (uM) | ID | assay IC50 (nM) | assay IC50 (uM) |
| C-1 | 13 | 9.8 | I-1365 | 42 | n.d. |
| I-376 | 220 | n.d. (not determined) | I-1453 | 35 | n.d. |
| I-377 | 93 | n.d. | I-1274 | 10 | 5.0 |
| I-566 | 58 | >20 | I-1275 | 22 | 5.2 |
| I-567 | 120 | >20 | I-1278 | 16 | 12 |
| I-1272 | 15 | 5.5 | I-1282 | 19 | 11 |
| I-1271 | 5 | 2.9 | I-1277 | 31 | 18 |
| I-1451 | 14 | n.d. | | | |

It was confirmed that various stapled peptides can inhibit cell proliferation. For example, in an assay assessing COLO320 viability, IC50 for I-1271, I-1274, I-1278 and C-1 demonstrated an IC50 of 900 nM, 3.4 uM, 2.4 uM, and 4.1 uM, respectively.

As confirmed herein, certain amino acid residues (e.g., Cys/Cys) and/or staple structures (e.g., as in I-1271, I-1272, I-1274, I-1275, etc.) provide stronger binding and activities (e.g., inhibition of cell proliferation) compared to other stapled peptides.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described in the present disclosure. It is, therefore, to be understood that the foregoing embodiments are presented by way of example only and that, provided technologies, including those to be claimed, may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An agent having the structure of 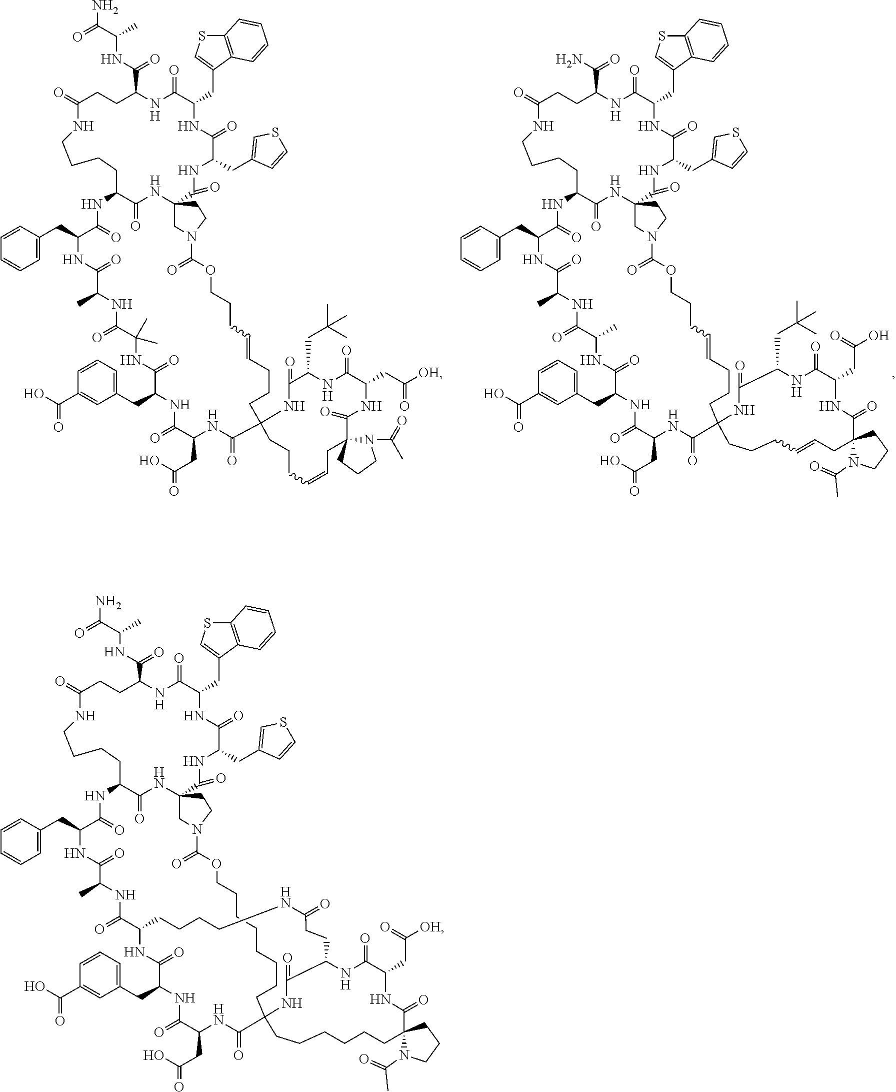 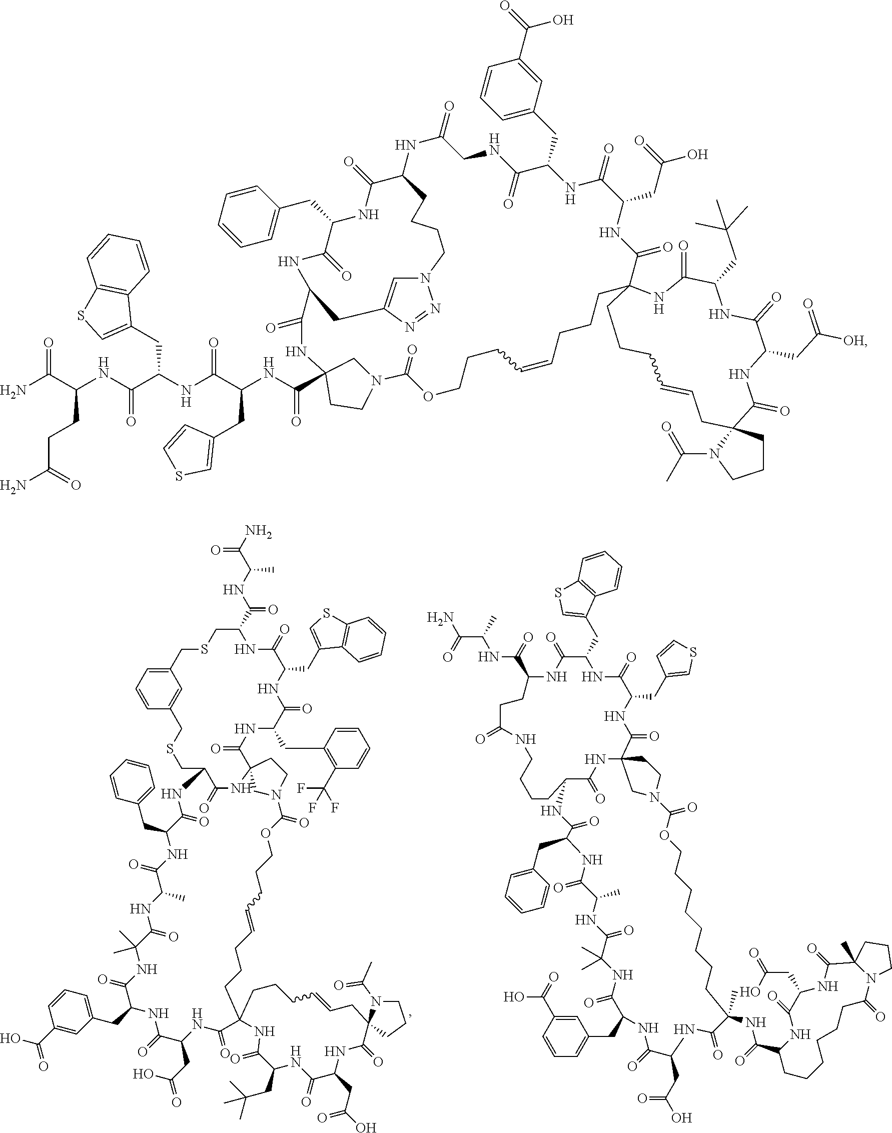 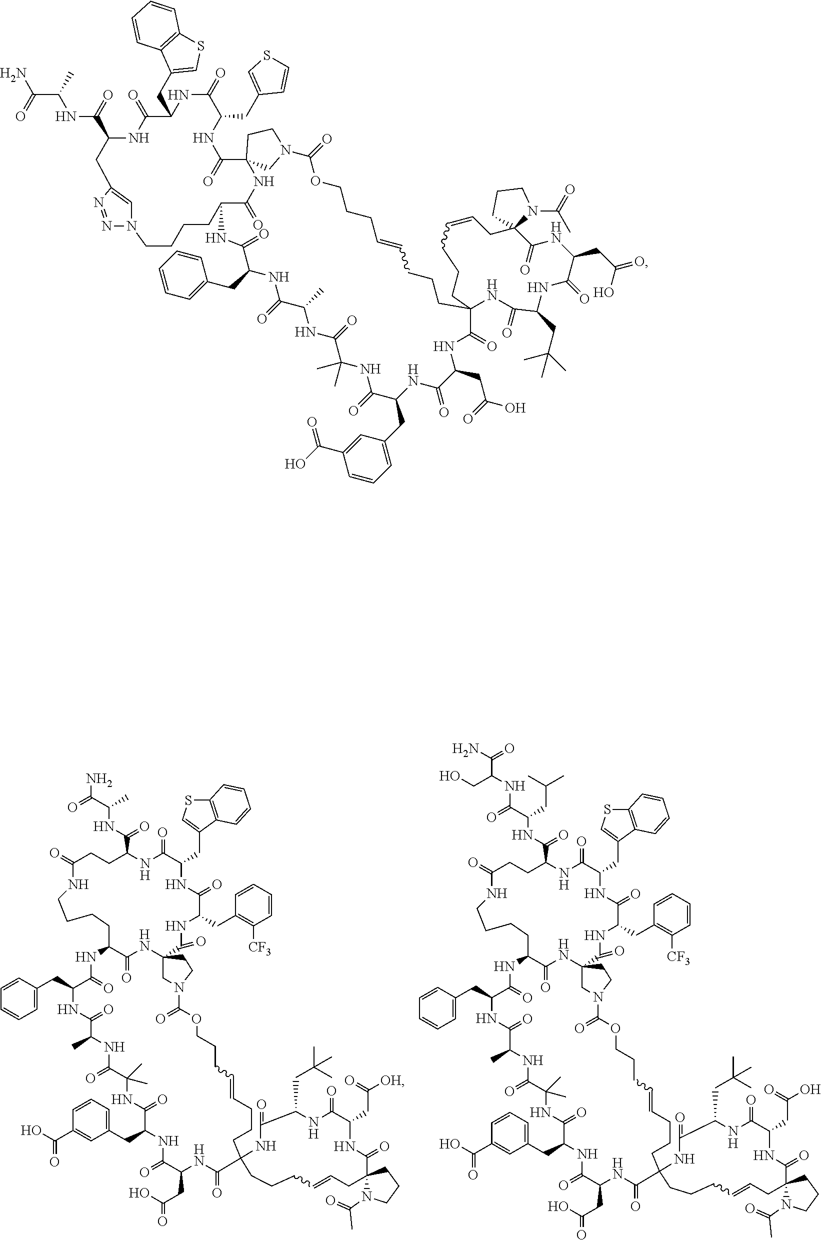 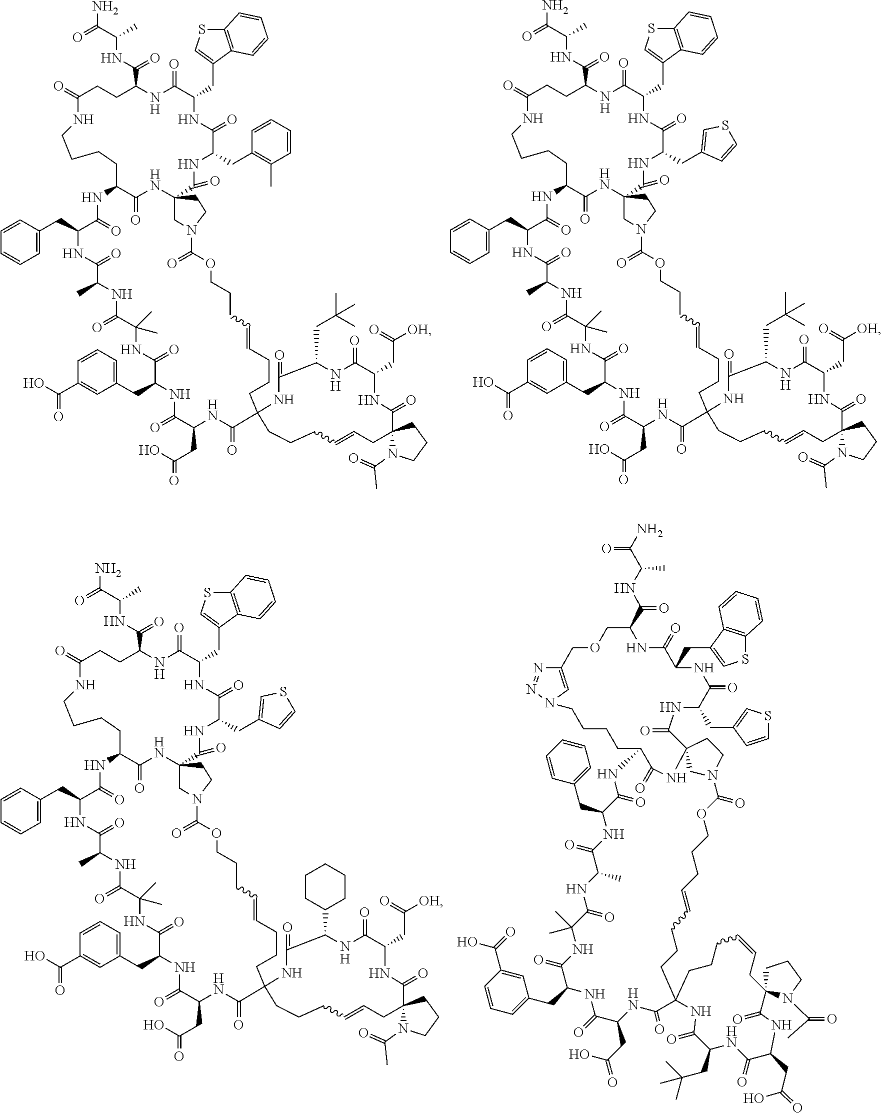 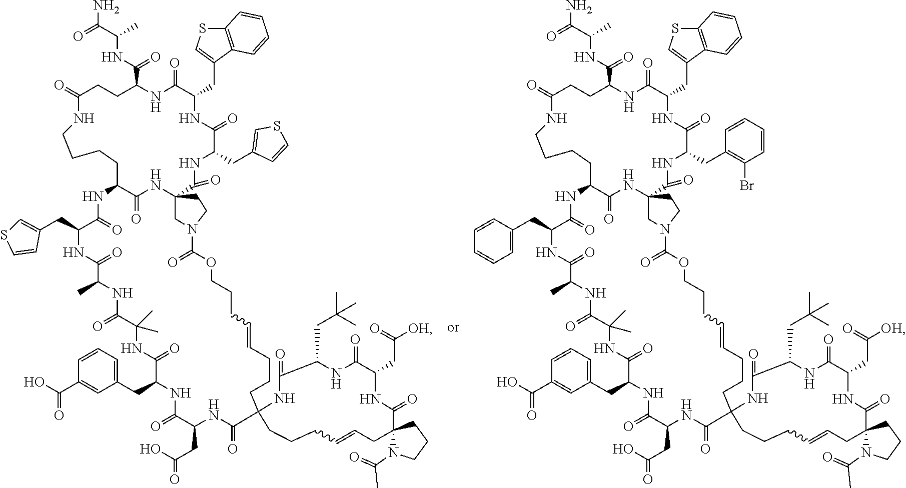
or a salt thereof.

2. An agent, wherein the agent is or comprises:
[X]ₚX¹X²X³X⁴X⁵X⁶X⁷X⁸X⁹X¹⁰X¹¹X¹²X¹³X¹⁴[X¹⁵]ₚ₁₅[X¹⁷]ₚ₁₇[X]_{p′,}
wherein:
each of p15, p16 and p17 is independently 0 or 1;
each of p and p′ is independently 0-10; and
each of X, X¹, X², X³, X⁴, X⁵, X⁶, X⁷, X⁸, X⁹, X¹⁰, X¹¹, X¹², X³, X¹⁴, X¹⁵, X¹⁶, and X¹⁷ is independently an amino acid residue.

3. The agent of claim 4, wherein the agent is or comprises a peptide comprising:
[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,
wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group; and
each of X⁹, X¹² and X¹³ comprises a side chain comprising an optionally substituted aromatic group.

4. The agent of any one of the preceding claims, the agent comprises three or more staples within 10-20 amino acid residues.

5. The agent of any one of the preceding claims, wherein five of X, X⁰, X³, X⁴, X⁷, X¹⁰, X¹¹ and X¹⁴ are each independently an amino acid residue suitable for stapling, or are each independently stapled.

6. The agent of any one of claims 1-5, wherein X¹ and X⁴ are connected by a staple.

7. The agent of any one of claims 1-5, wherein X⁰ and X⁴ are connected by a staple.

8. The agent of any one of claims 1-7, wherein X⁴ and X¹ are connected by a staple.

9. The agent of any one of claims 1-8, wherein X¹⁰ and X¹⁴ are connected by a staple.

10. The agent of any one of claims 1-9, wherein X⁷ and X¹⁰ are connected by a staple.

11. The agent of any one of claims 1-10, wherein X⁷ and X¹⁴ are connected by a staple.

12. The agent of any one of the preceding claims, wherein the agent comprises a N-terminal group.

13. The agent of any one of the preceding claims, wherein X¹ is a residue of an amino acid having the structure of formula A-I, A-II or A-III, wherein R^{a1} and R^{a3} are taken together with their intervening atom(s) to form an optionally substituted 3-10 membered ring having 0-5 heteroatoms in addition to the intervening atom(s).

14. The agent of any one of the preceding claims, wherein X¹ is PL3.

15. The agent of any one of the preceding claims, wherein X⁴ is a residue of an amino acid that comprises an olefin.

16. The agent of any one of the preceding claims, wherein X⁴ is B5.

17. The agent of any one of the preceding claims, wherein X¹⁰ is a residue of an amino acid that comprises an optionally substituted carboxyl group, an optionally substituted amino group, an azidyl group, an optionally substituted alkynyl group, or an optionally substituted thiol group.

18. The agent of any one of the preceding claims, wherein X¹⁰ is Lys.

19. The agent of any one of the preceding claims, wherein X¹¹ is a residue of an amino acid that comprises an olefin.

20. The agent of any one of the preceding claims, wherein X¹¹ is PyrS2.

21. The agent of any one of the preceding claims, wherein X¹⁴ is a residue of an amino acid that comprises a carboxyl group, an amino group, an azidyl group, an alkynyl group, or a thiol group.

22. The agent of any one of the preceding claims, wherein X¹⁴ is GlnR.

23. The agent of any one of the preceding claims, wherein one of X¹⁰ and X¹⁴ is a residue of an amino acid that comprises a carboxyl group, and the other is a residue of an amino acid that comprises an amino group.

24. The agent of any one of the preceding claims, wherein X¹⁰ and X¹⁴ are connected by a staple, wherein the staple comprises —C(O)N(R′)—.

25. The agent of any one of the preceding claims, wherein X² comprises a side chain comprising an acidic group.

26. The agent of any one of claims 1-24, wherein X² is Asp, Ala, Asn, Glu, Npg, Ser, Hse, Val, S5, S6, AcLys, TfeGA, aThr, Aad, Pro, Thr, Phe, Leu, PL3, Gln, isoGlu, MeAsn, isoDAsp, RbGlu, SbGlu, AspSH, Ile, SbMeAsp, RbMeAsp, aMeDAsp, OAsp, 3COOHF, NAsp, 3Thi, NGlu, isoDGlu, BztA, Tle, Aib, MePro, Chg, Cha, or DipA.

27. The agent of any one of the preceding claims, wherein X³ comprises one or two hydrophobic side chains.

28. The agent of any one of claims 1-26, wherein X³ is Npg, Ile, Asp, Cha, DipA, Chg, Leu, B5, Cba, S5, Ala, Glu, AllylGly, nLeu, Ser, B6, Asn, B4, GlnR, Val, [Phc][Allyl]Dap, Hse, [Bn][Allyl]Dap, 1MeK, R⁵, Phe, CypA, CyLeu, Pff, DiethA, Tyr, Trp, Aib, Phg, OctG, MorphNva, F2PipNva, [Piv][Allyl]Dap, [CyCO][Allyl]Dap, Lys, or S3.

29. The agent of any one of the preceding claims, wherein X⁵ comprises a side chain comprising an acidic group.

30. The agent of any one of claims 1-28, wherein X⁵ is selected from Asp, 3COOHF, TfeGA, Gln, [CH2CMe2CO2H]TriAzDap, Thr, Glu, 2OH3COOHF, 40H3COOHF, 4COOHF, 2COOHF, His, Tyr, 5F3Me2COOHF, 4F3Me2COOHF, 5F3Me3COOHF, 4F3Me3COOHF, 3F2COOHF, Val, Ser, Trp, Asn, Ala, Arg, dGlu, aThr, hTyr, 3cbmf, Leu, Phe, Lys, and Ile.

31. The agent of any one of the preceding claims, wherein X⁶ comprises a side chain comprising an acidic group.

32. The agent of any one of the preceding claims, wherein X⁶ is 3COOHF, TfeGA, or Asp.

33. The agent of any one of the preceding claims, wherein X⁷ is a hydrophobic amino acid residue.

34. The agent of any one of claims 1-32, wherein X⁷ is selected from Aib, Ala, MorphGln, Gln, Ser, iPrLys, nLeu, Cha, Hse, Npg, Val, CyLeu, Thr, Phe, Acp, Asn, DaMeS, aMeDF, Leu, Cpg, Cbg, Me2Gln, Met2O, AcLys, His, aMeL, DaMeL, aMeV, aMeS, and aMeF.

35. The agent of any one of the preceding claims, wherein X¹¹ is a hydrophobic amino acid residue.

36. T The agent of any one of claims 1-34, wherein X¹¹ is selected from Ala, Aib, Cpg, Val, Leu, Gln, Lys, Asp, Glu, Aad, nLeu, Cba, Ser, Thr, aThr, MorphGln, Phe, hPhe, hTyr, and AcLys.

37. The agent of any one of the preceding claims, wherein X⁹ comprises a side chain which is or comprises an optionally substituted aromatic group.

38. The agent of any one of claims 1-36, wherein X⁹ is AA9, Phe, Ala, Lys, 3COOHF, Aib, 2NapA, nLeu, 2Thi, Tyr, 3Thi, 4FF, 4ClF, 4BrF, 3FF, 3ClF, 3BrF, 2FF, 30MeF, 4CNF, 3CNF, 4MeF, 3MeF, Aic, RbiPrF, SbiPrF, RbiPrDF, RbMeXylA, RbMeXylDA, Cba, CypA, BztA, 1NapA, Trp, Leu, Ile, Ser, Chg, Hse, 4TriA, 3F3MeF, Thr, His, Val, Asn, Gln, 2Cpg, SbMeXylA, or SbMeXylDA.

39. The agent of any one of the preceding claims, wherein X¹² comprises a side chain which is or comprises an optionally substituted aromatic group.

40. The agent of any one of claims 1-38, wherein X¹² is 3Thi, Phe, 2F3MeF, PyrS2, 2ClF, hnLeu, BztA, 2Thi, 2MeF, 2FF, 34ClF, Lys, nLeu, 2COOHF, 2PhF, hCbA, hCypA, hCha, CypA, hPhe, DipA, HepG, Dap7Abu, hhLeu, hhSer, HexG, [2IAPAc]2NH2F, Ala, Abu, Leu, hLeu, Npg, Cpa, PyrS1, [Bnc]2NH2F, [Phc]2NH2F, [BiPh]2NH2F, [3PyAc]2NH2F, Nva, Cba, ChA, 2FurA, 20MeF, 2BrF, 2CNF, 2NO2F, 2PyrA, 3PyrA, 4PyrA, His, 1NapA, Val, Ile, Chg, DiethA, OctG, 2cbmF, c6Phe, [MePipAc]2NH2F, or [2PyCypCO]2NH2F.

41. The agent any one of the preceding claims, wherein the side chain of X¹³ comprises an optionally substituted aromatic group.

42. The agent of any one of claims 1-40, wherein X¹³ is selected from BztA, 34ClF, 2NapA, 3BrF, 34MeF, 3Thi, Phe, GlnR, 34MeF, 2NapA and Lys.

43. The agent of any one of the preceding claims, wherein p15 is 1.

44. The agent of any one of the preceding claims, wherein X⁵ comprises a hydrophobic side chain.

45. The agent of any one of the preceding claims, wherein the peptide forms a structure that comprises a helix.

46. The agent of any one of the preceding claims, wherein the peptide binds to beta-catenin.

47. The agent of any one of the preceding claims, wherein the peptide binds to a polypeptide whose sequence is or comprising SEQ ID NO: 2, or a fragment thereof:
(SEQ ID NO: 2)
SVLFYAITTLHNLLLHQEGAKMAVRLAGGLQKMVALLNKTNVKFLAITTD
CLQILAYGNQESKLIILASGGPQALVNIMRTYTYEKLLWTTSRVLKVLSV
CSSNKPAIVEAGGMQALGLHLTDPSQRLVQNCLWTLRNLSDAATKQEGME
GLLGTLVQLLGSDDINVVTCAAGILSNLTCNNYKNKMMVCQVGGIEALVR
T.

48. The agent of any one of the preceding claims, wherein the peptide binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, R342, K345, V346, V349, Q375, R376, Q379, N380, L382, W383, R386, N387, D413, N415, V416, T418, and C419.

49. The agent of any one of the preceding claims, wherein a double bond of a (i, i+7) staple is E.

50. The agent of any one of the preceding claims, wherein a double bond of a (i, i+7) staple is Z.

51. The agent of any one of the preceding claims, wherein a double bond of a (i, i+2), (i, i+3) or (i, i+4) staple is E.

52. The agent of any one of the preceding claims, wherein a double bond of a (i, i+2), (i, i+3) or (i, i+4) staple is Z.

53. The agent of any one of the preceding claims, wherein a carbon atom bonded to two staples (e.g., in B5) is of R configuration.

54. The agent of any one of any one of the preceding claims, wherein a carbon atom bonded to two staples (e.g., in B5) is of S configuration.

55. An agent, having the structure of SP-1-1, SP-1-2, SP-1-3, SP-1-4, SP-1-5, SP-1-6, SP-1-7, SP-1-8, SP-2-1, SP-2-2, SP-2-3, SP-2-4, SP-2-5, SP-2-6, SP-2-7, SP-2-8, SP-3-1, SP-3-2, SP-4-1, SP-4-2, SP-4-3, SP-4-4, SP-4-5, SP-4-6, SP-4-7, SP-4-8, SP-5-1, SP-5-2, SP-5-3, SP-5-4, SP-5-5, SP-5-6, SP-5-7, SP-5-8, SP-6, SP-7-1, SP-7-2, SP-7-3, SP-7-4, SP-7-5, SP-7-6, SP-7-7, SP-7-8, SP-8-1, SP-8-2, SP-8-3, SP-8-4, SP-8-5, SP-8-6, SP-8-7, SP-8-8, SP-9-1, SP-9-2, SP-9-3, SP-9-4, SP-9-5, SP-9-6, SP-9-7, SP-9-8, SP-10-1, SP-10-2, SP-10-3, SP-10-4, SP-10-5, SP-10-6, SP-10-7, SP-10-8, SP-11-1, SP-11-2, SP-11-3, SP-11-4, SP- 11-5, SP-11-6, SP-11-7, SP-11-8, SP-12-1, SP-12-2, SP-12-3, SP-12-4, SP-12-5, SP-12-6, SP-12-7, SP-12-8, SP-13-1, SP-13-2, SP-13-3, SP-13-4, SP-13-5, SP-13-6, SP-13-7, SP-13-8, SP-14-1, SP-14-2, SP-14-3, SP- 14-4, SP-14-5, SP-14-6, SP-14-7, SP-14-8, SP-15-1, SP-15-2, SP-15-3, SP-15-4, SP-15-5, SP-15-6, SP-15-7, SP-15-8, or a salt thereof.

56. An agent having the structure of 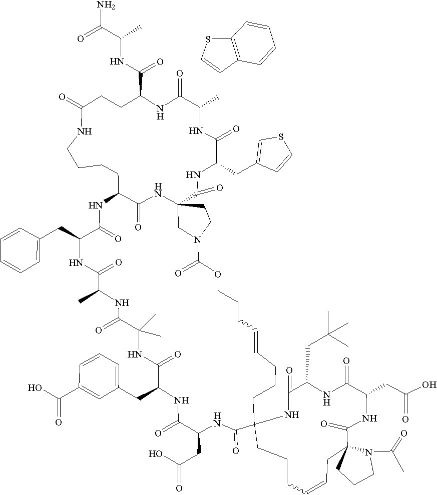 [SMILES: CC(=O)N1CCC[C@]12CC=CCCCC1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]
or a salt thereof.

57. An agent having the structure of 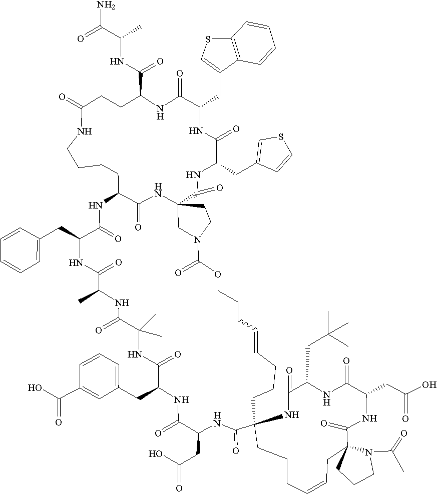 [SMILES: CC(=O)N1CCC[C@]12C/C=C\CCC[C@]1(CCCC=CCCCOC(=O)N3CC[C@@]4(C3)NC(=O)[C@H](CCCCNC(=O)CC[C@@H](C(=O)N[C@@H](C)C(N)=O)NC(=O)[C@H](Cc3csc5ccccc35)NC(=O)[C@H](Cc3ccsc3)NC4=O)NC(=O)[C@H](Cc3ccccc3)NC(=O)[C@H](C)NC(=O)C(C)(C)NC(=O)[C@H](Cc3cccc(C(=O)O)c3)NC(=O)[C@H](CC(=O)O)NC1=O)NC(=O)[C@H](CC(C)(C)C)NC(=O)[C@H](CC(=O)O)NC2=O]
or a salt thereof.

58. The agent of any one of claims 56-57, wherein the agent has the same retention time under a HPLC condition as I-66 prepared as described in Example 9, wherein the HPLC condition can separate I-66 and I-67 prepared as described in Example 9.

59. The agent of any one of claims 56-57, wherein the agent shows a retention time of about 15.3 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

60. The agent of any one of claims 56-59, wherein the agent elutes in a single peak with I-66 prepared as described in Example 9 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

61. The agent of any one of claims 56-60, characterized in that the agent shows ¹H NMR peaks that overlap with the peaks between about 5.1-5.7 in FIG. 6 under the same or comparable conditions.

62. The agent of any one of claims 56-60, characterized in that the agent shows the same ¹H NMR peaks between about 5.1-5.7 as FIG. 6 under the same or comparable conditions.

63. The agent of any one of claims 56-60, characterized in that in its ¹H NMR spectrum, the peaks corresponding to ¹H bonded to carbon atoms overlap with peaks in FIG. 6 under the same or comparable conditions.

64. The agent of any one of claims 56-60, characterized in that its ¹H NMR spectrum overlaps with peaks in FIG. 6 under the same or comparable conditions.

65. The agent of any one of claims 56-57, wherein the agent has the same retention time under a HPLC condition as I-67 prepared as described in Example 9, wherein the HPLC condition can separate I-66 and I-67 prepared as described in Example 9.

66. The agent of any one of claims 56-57, wherein the agent shows a retention time of about 16.2 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

67. The agent of any one of claims 56-59, wherein the agent elutes in a single peak with I-67 prepared as described in Example 9 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

68. The agent of any one of claims 56-59 and 65-67, characterized in that the agent shows ¹H NMR peaks that do not overlap with the peaks between about 5.1-5.7 in FIG. 6 under the same or comparable conditions.

69. The agent of any one of claims 56-59 and 65-67, characterized in that the agent does not show the same ¹H NMR peaks between about 5.1-5.7 as FIG. 6 under the same or comparable conditions.

70. The agent of any one of claims 56-59 and 65-67, characterized in that in its ¹H NMR spectrum, the peaks corresponding to ¹H bonded to carbon atoms do not all overlap with peaks in FIG. 6 under the same or comparable conditions.

71. The agent of any one of claims 56-60, characterized in that its ¹H NMR spectrum does not overlap with peaks in FIG. 6 under the same or comparable conditions.

72. The agent of any one of any one of the preceding claims, wherein a carbon atom bonded to two staples (e.g., in B5) is of S configuration.

73. An agent having the structure of formula I:
R^{N}-LP-L^{AA1}-L^{P2}-L^{AA2}-L^{P3}-L^{AA3}-L^{P4}-L^{AA4}-L^{P5}-L^{AA5}-L^{P6}-L^{AA6}L^{P7}-R^{C}, I
or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein LPL, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms; or
an agent having the structure of formula I:
R^{N}-L^{P1}-L^{AA1}-L^{P2}-L^{AA2}-L^{P3}-L^{AA3}-L^{P4}-L^{AA4}-L^{P5}-L^{AA5}-L^{P6}-L^{AA6}-L^{P7}-R^{C}, I
or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}wherein R^{AA1} is —CO₂R or —SO₂R;
L^{AA2} is L^{AR}, wherein a methylene unit is replaced with —C(R′)(R^{AS})—, wherein R^{AS} is -L^{AS2}-R^{AA2}, wherein R^{AA2} is —CO₂R, or —SO₂R;
L^{AA3} is L^{AR}, wherein a methylene unit is replaced with —C(R′)(R^{AS})—, wherein R^{AS} is -L^{AS3}-R^{AA3}, wherein R^{AA3} is R′;
L^{AA4} is L^{AR}, wherein a methylene unit is replaced with —C(R′)(R^{AS})—, wherein R^{AS} is -L^{AS4}-R^{AA4}, wherein R^{AA}A4 is an optionally substituted group selected from 6-14 membered aryl or 5-14 membered heteroaryl having 1-6 heteroatoms;
L^{AA5} is L^{AR}, wherein a methylene unit is replaced with —C(R′)(R^{AS})—, wherein R^{AS} is -L^{AS5}-R^{AA5}, wherein R^{AA}A5 is an optionally substituted group selected from 6-14 membered aryl or 5-14 membered heteroaryl having 1-6 heteroatoms;
L^{AA6} is L^{AR}, wherein a methylene unit is replaced with —C(R′)(R^{AS})—, wherein R^{AS} is -L^{AS6_}R^{AA6}, wherein R^{AA}A6 is an optionally substituted group selected from 6-14 membered aryl or 5-14 membered heteroaryl having 1-6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{AR} is independently an optionally substituted, bivalent C₁-C₆ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AS} is independently -L^{AS}-R′;
each L^{AS} is independently a covalent bond or an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

74. The agent of any one of the preceding claims, wherein each olefin double bond in a staple is independently and optionally converted into a single bond.

75. The agent of any one of the preceding claims, having a diastereopurity of about 80%, 85%, 90%, 91%, 92%, 93%, 94%, 95%, 96%, 97%, 98%, 99% or more, or having a purity of about 80%, 85%, 90%, 91%, 92%, 93%, 94%, 95%, 96%, 97%, 98%, 99% or more.

76. A pharmaceutical composition, comprising or delivering an agent or amino acid of any one of the preceding claims, and a pharmaceutically acceptable carrier.

77. A composition selected from Table E2 or Table E3, or a pharmaceutical composition, comprising or delivering one or more or all peptide agents in a composition selected from Table E2 or Table E3, and a pharmaceutically acceptable carrier.

78. The composition of any one of the preceding claims, comprising an agent comprising one or more staples each independently comprises one or more olefin double bond, wherein the ratio of the two stereoisomers of an olefin double bond in a staple is about 2:1, 3:1, 4:1, 5:1, 6:1, 7:1, 8:1, 9:1, 10:1, 15:1, 20:1, 30:1, 40:1, 50:1 or more.

79. A method, comprising
a) preparing a first compound comprising two moieties each of which independently comprises an olefin double bond;
b) providing a second compound by stapling the two moieties by olefin metathesis of an olefin double bond of one moiety with an olefin double bond of the other to form a first-formed staple;
c) add one or more additional moieties to the second compound to provide a third compound which comprising two moieties each of which independently comprises an olefin double bond; and
d) providing a fourth compound by stapling the two moieties in the third compound by olefin metathesis of an olefin double bond of one moiety with an olefin double bond of the other to form a second-formed staple.

80. A method for modulating beta-catenin interaction with a partner in a system, comprising contacting beta-catenin with an agent or composition of any one of the preceding claims; or
a method for modulating beta-catenin interaction with a partner in a system, comprising administering or delivering to the system an agent or composition of any one of the preceding claims; or
a method for modulating a TCF-beta-catenin interaction in a system, comprising contacting beta-catenin with an agent or composition of any one of the preceding claims; or
a method for modulating a TCF-beta-catenin interaction in a system, comprising administering or delivering to the system an agent or composition of any one of the preceding claims; or
a method for inhibiting beta-catenin dependent cell proliferation, comprising administering or delivering to the system an agent or composition of any one of the preceding claims; or
a method for modulating WNT/beta-catenin pathway in a system, comprising administering or delivering to the system an agent or composition of any one of the preceding claims, wherein expression of a nucleic acid is modulated; or
a method, comprising administering or delivering to the system an agent or composition of any one of the preceding claims, wherein level of a transcript of a nucleic acid and/or a product thereof is modulated; or
a method, comprising administering or delivering to the system an agent or composition of any one of the preceding claims, wherein expression of a nucleic acid is modulated.

81. A method for treating or preventing a condition, disorder or disease associated with beta-catenin interaction with a partner in a subject, comprising administering or delivering to the subject an effective amount of an agent or composition of any one of the preceding claims, preferably wherein the partner is TCF7, LEF1, TCF7L1, TCF7L2, Axin1, Axin2, or APC.

82. A method for treating cancer in a subject, comprising administering or delivering to the subject an effective amount of an agent or composition of any one of the preceding claims.

83. The method of any one of the preceding claims, comprising administering or deliver to a subject a second therapeutic agent or therapy.

84. The method of any one of the preceding claims, wherein a second therapeutic agent is or comprises a chemotherapy agent, a hormone therapy agent, an immunotherapy agent, a checkpoint inhibitor, an antibody, a CTLA-4, PD-1 or PD-L1 inhibitor, or a cell, or a second therapy is or comprises surgery, chemotherapy, radiotherapy, hormone therapy, stem cell or bone marrow transplant, immunotherapy, T-cell therapy, or CAR T-cell therapy.

85. The method of any one of the preceding claims, comprising assessing expression of a nucleic acid.

86. A compound having the structure of formula PA:
N(R^{PA})(R^{a1})-L^{a1}-C(R^{a2})(R^{a3})-L^{a2}-C(O)R^{PC}, PA
or a salt thereof, wherein:
R^{PA} is —H or an amino protecting group;
each of R^{a1} and R^{a3} is independently -L^{a}-R′;
R^{a2} is -L^{aa}-C(O)R^{PS};
each of L^{a}, L^{a1} and L^{a2} is independently L;
—C(O)R^{PS} is optionally protected or activated —COOH;
—C(O)R^{PC} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆-30 arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms; or
a compound having the structure of: 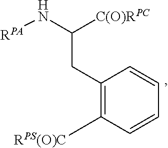
or a salt thereof, wherein:
R^{PA} is —H or an amino protecting group;
—C(O)R^{PS} is optionally protected or activated —COOH; and
—C(O)R^{PC} is optionally protected or activated —COOH; or
a compound having the structure of: 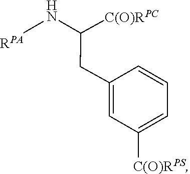
or a salt thereof, wherein:
R^{PA} is —H or an amino protecting group;
—C(O)R^{PS} is optionally protected or activated —COOH; and
—C(O)R^{PC} is optionally protected or activated —COOH.

87. An agent, compound, method, or composition of any one of Embodiments 1-2401.
